(19) United States
(12) Patent Application Publication
(10) Pub. No.: US 2023/0391765 A1
(43) Pub. Date: Dec. 7, 2023

(54) HETEROBIFUNCTIONAL COMPOUNDS AS DEGRADERS OF ENL

(71) Applicants: ICAHN SCHOOL OF MEDICINE AT MOUNT SINAI, New York, NY (US); VAN ANDEL RESEARCH INSTITUTE, Grand Rapids, MI (US)

(72) Inventors: Jian Jin, New York, NY (US); H. Umit Kaniskan, New York, NY (US); Lihuai Qin, New York, NY (US); Hong Wen, Grand Rapids, MI (US); Xiaobing Shi, Grand Rapids, MI (US); Longxia Xu, Grand Rapids, MI (US); Zhaoyu Xue, Grand Rapids, MI (US)

(21) Appl. No.: 18/032,758

(22) PCT Filed: Oct. 19, 2021
(86) PCT No.: PCT/US2021/055574

§ 371 Date: Apr. 19, 2023

Related U.S. Application Data
(60) Provisional application No. 63/094,771, filed on Oct. 21, 2020.

(51) Int. Cl. C07D 417/14; C07D 401/14; C07D 413/14; A61P 35/02

(52) U.S. Cl. CPC C07D 417/14; A61P 35/02; C07D 413/14; C07D 401/14

(57) ABSTRACT

Disclosed are Eleven-Nineteen Leukemia (ENL) degradation/disruption compounds including a ENL ligand, a degradation/disruption tag and a linker, and methods for use of such compounds in the treatment of ENL-mediated diseases.

### TECHNICAL FIELD

This disclosure relates to bivalent compounds (e.g., heterobifunctional compounds) which degrade and/or disrupt Eleven-Nineteen Leukemia (ENL), compositions comprising one or more of the bivalent compounds, and methods of use thereof for the treatment of ENL-mediated diseases in a subject in need thereof. The disclosure also relates to methods for designing such bivalent compounds.

### BACKGROUND OF THE INVENTION

Eleven-Nineteen Leukemia (ENL, also known as MLLT1 or YEATS1) is a transcriptional co-regulator that recruits transcription machinery to target genes through its chromatin reader function. ENL and its paralogue ALL1-Fused Gene From Chromosome 9 (AF9, also known as MLLT3 or YEATS3) associate with the super elongation complex (SEC) and the complex of the histone H3K79 methyltransferase DOT1L (Biswas et al., 2011; He et al., 2011), both of which play important roles in regulation of transcription elongation by RNA polymerase II (Bitoun et al., 2007; He et al., 2010; Lin et al., 2010; Mohan et al., 2010a; Mueller et al., 2007; Mueller et al., 2009; Okada et al., 2005; Yokoyama et al., 2010). Both ENL and AF9 proteins contain a N-terminal YEATS domain, which is an evolutionarily conserved domain that recognizes acylated lysine on histone H3 tail (Hsu et al., 2018; Klein et al., 2018; Li et al., 2016; Li et al., 2014; Mi et al., 2017; Shanle et al., 2015; Wan et al., 2017; Zhang et al., 2016).

ENL plays a vital role in the progression and maintenance of certain subtypes of acute leukemia, mixed lineage leukemia (MLL)-rearranged leukemia in particular (Erb et al., 2017; Wan et al., 2017). The MLL gene (also known as MLL1, ALL-4, or KMT2A) is disrupted by recurrent chromosomal rearrangements in a subgroup of high-risk acute leukemias that have unique clinical and biological features (Hess, 2004; Meyer et al., 2013; Meyer et al., 2009; Rao and Dou, 2015). MLL rearrangements account for approximately 10% of all human leukemias, most frequently in infant leukemias (Marschalek, 2015; Meyer et al., 2013). These patients have a dismal prognosis and a particularly poor response to standard treatments (Biondi et al., 2000; Pieters et al., 2007; Pui et al., 2009). Therefore, development of effective therapies for this leukemia subtype is urgently needed. Leukemogenic translocations of the MLL gene lead to in-frame fusions between the N-terminus of the MLL protein and the C-terminus of a fusion partner, and these fusion proteins are known to function as “drivers” of the diseases (Abramovich and Humphries, 2005; Armstrong et al., 2002; Artinger et al., 2013; Deshpande et al., 2012; Ferrando et al., 2002; Jude et al., 2007; Slany, 2005; Yu et al., 1995). Strikingly, among the over 70 MLL fusions characterized, a small subset of fusions accounts for most leukemogenic cases. Over 90% of MLL rearrangements in acute lymphoblastic leukemia (ALL) and 70% in acute myeloid leukemia (AML) involve only 4-5 fusion partners, all of which are subunits of the SEC and/or DOT1L complexes that ENL and AF9 reside in (Ayton and Cleary, 2001; Krivtsov and Armstrong, 2007; Meyer et al., 2013; Meyer et al., 2006; Mohan et al., 2010b). It is believed that each complex component, when fused to MLL, “hijacks” the SEC or DOT1L complex to the MLL target loci, promoting aberrant gene activation that leads to leukemogenesis (Deshpande et al., 2012). In recent studies, ENL, but not AF9, is identified as a cancer-specific acute leukemia dependency (Erb et al., 2017; Wan et al., 2017). ENL depletion or disrupting the interaction between its YEATS domain and histone acetylation leads to inhibition of oncogenic gene expression programs and suppression of leukemia progression both in vitro and in vivo (FIG. 1).

In addition, hotspot ENL YEATS domain mutations have been identified in Wilms' tumor patients (Gadd et al., 2017; Perlman et al., 2015). The reader function of the YEATS domain is indispensable for these gain-of-function mutations to aberrantly activate the expression of genes essential for proper kidney development and derail the cell-fate decision (Wan et al., 2020).

All these studies suggest that ENL and its YEATS domain are attractive therapeutic target for certain types of human cancer. Efforts in developing ENL YEATS domain inhibitors led to the recent publications of acetyl-lysine competitive small molecules, peptide-mimic chemical probes and ligands from cell-based screen, demonstrating that the YEATS domain is pharmacologically tractable (Asiaban et al., 2020; Christott et al., 2019; Heidenreich et al., 2018; Li et al., 2018; Moustakim et al., 2018a; Ni et al., 2019). One of the recently reported ENL YEATS small molecule inhibitors, SGC-iMLLT, can effectively block the interaction between the ENL YEATS domain and acetylated histone H3 in vitro and in cells (Christott et al., 2019; Moustakim et al., 2018a). However, while SGC-iMLLT is an excellent chemical probe with nanomolar level of binding affinity to the ENL YEATS domain in vitro, it is largely ineffective in inhibiting the growth of ENL-dependent MLL-rearranged leukemia cells (Christott et al., 2019; Moustakim et al., 2018a). The lack of a significant effect by SGC-iMLLT in cells is in contrast to the effect of ENL knockout (KO) via CRISPR-Cas9 (Erb et al., 2017; Wan et al., 2017). Therefore, a new therapeutic strategy targeting ENL is needed. Here, we present small-molecule degraders of ENL, which pharmacologically degrade ENL protein in cells and tumors and more likely phenocopy the effects of ENL KO, as novel therapeutics for treating ENL-dependent diseases including cancers.

### SUMMARY OF THE INVENTION

The present disclosure relates generally to bivalent compounds (e.g., bi-functional compounds), which degrade and/or disrupt ENL and to methods for the treatment of ENL-mediated diseases (i.e., a disease which depends on ENL; overexpresses ENL; depends on ENL activity; or includes elevated levels of ENL activity relative to a wild-type tissue of the same species and tissue type). It is important to note, because the ENL degraders/disruptors have dual functions (enzyme inhibition plus protein degradation/disruption), the bivalent compounds of the present disclosure can be significantly more effective therapeutic agents than currently available ENL inhibitors, which inhibit the enzymatic activity of ENL, but do not affect ENL protein levels. The present disclosure further provides methods for identifying ENL degraders/disruptors as described herein.

More specifically, the present disclosure provides a bivalent compound including an ENL ligand conjugated to a degradation/disruption tag.

In some aspects, the ENL degraders/disruptors have the form “PI-linker-EL”, as shown below:

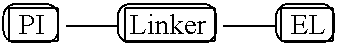

wherein PI (protein of interest) comprises an ENL ligand and EL (E3 ligase) comprises a degradation/disruption tag (e.g., E3 ligase ligand). Exemplary ENL ligands (PI), exemplary degradation/disruption tags (EL), and exemplary linkers (Linker) are illustrated below:

ENL Ligands

In an embodiment, ENL ligands include a moiety according to FORMULA 1:

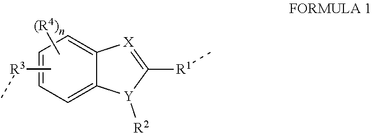

wherein
the “Linker” moiety of the bivalent compound is attached independently to R¹ or R³
X and Y are independently selected from C, O or N;

R¹ is selected from H, halogen, OR⁵, SR⁵, C₁-C₈ alkylene NR⁵R⁶, CH₂CH₂NR⁵R⁶, NR⁵R⁶, C(O)R⁵, C(O)OR⁵, C(S)OR⁵, C(O)NR⁵R⁶, S(O)R⁵, S(O)₂R⁵, S(O)₂NR⁵R⁶, NR⁷C(O)OR⁶, NR⁷C(O)R⁶, NR⁷S(O)R⁶, NR⁷S(O)₂R⁶, or unsubstituted or optionally substituted C₁-C₈ alkyl, C₁-C₈ haloalkyl, C₁-C₈ hydroxyalkyl, C₃-C₁₀ cycloalkyl, C₃-C₁₀ heterocyclyl, optionally substituted C₁-C₈alkoxyC₁-C₈alkyl, optionally substituted C₁-C₈alkylaminoC₁-C₈alkyl.

R² is independently selected from hydrogen, halogen, oxo, CN, NO₂, OR⁸, SR⁸, NR⁸R⁹, C(O)R⁸, C(O)OR⁸, C(S)OR⁸, C(O)NR⁸R⁹, S(O)R⁸, S(O)₂R⁸, S(O)₂NR⁸R⁹, NR¹⁰C(O)OR⁹, NR¹⁰C(O)R⁹, NR¹⁰S(O)R⁹, NR¹⁰S(O)₂R⁹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₃-C₈ heterocyclyl, optionally substituted aryl, and optionally substituted heteroaryl;

R³ is unsubstituted or optionally substituted with one or more groups selected from hydrogen, halogen, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR¹¹R¹², C(O)R¹¹, C(O)OR¹¹, C(O)NR¹¹R¹², S(O)R¹¹, S(O)₂R¹¹, S(O)₂N¹¹R¹², NR¹³C(O)OR¹², NR¹³C(O)R¹², NR¹³S(O)R¹², NR¹³S(O)₂R¹², optionally substituted C₆-C₁₀ aryl and optionally substituted C₅-C₁₀ heteroaryl.

each R⁴ is independently selected from null, hydrogen, halogen, oxo, CN, NO₂, OR¹⁴, SR¹⁴, NR¹⁴R¹⁵, OCOR¹⁴, OCO₂R¹⁴, OCONR¹⁴R¹⁵, COR¹⁴, CO₂R¹⁵, CONR¹⁴R¹⁵, SOR¹⁴, SO₂R¹⁴, SO₂NR¹⁴R¹⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;

R⁵, R⁶, R⁷, R⁸, R⁹, R¹⁰ R¹¹, R¹², R¹³ R¹⁴, R¹⁵ are independently selected from H, C₁-C₈ alkyl, C₁-C₈ haloalkyl, C₁-C₈ hydroxyalkyl, C₃-C₁₀ cycloalkyl, C₃-C₁₀ heterocyclyl, C(O) C₁-C₈ alkyl, C(O) C₁-C₈ haloalkyl, C(O) C₁-C₈ hydroxyalkyl, C(O) C₃-C₁₀ cycloalkyl, C(O) C₃-C₁₀ heterocyclyl, optionally substituted C₆-C₁₀ aryl or C₅-C₁₀ heteroaryl.

R⁵ and R⁶, R⁶ and R⁷, R⁸ and R⁹, R⁸ and R¹⁰, R⁹ and R¹⁰, R¹¹ and R¹², R¹¹ and R¹³, R¹² and R¹³, R¹⁴ and R¹⁵, together with the nitrogen atom to which they connected can independently form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring.
n is independently selected from 0, 1, 2, 3, 4 and 5;
and pharmaceutically acceptable salts thereof.

In an embodiment, ENL ligands include a moiety according to FORMULA 1A

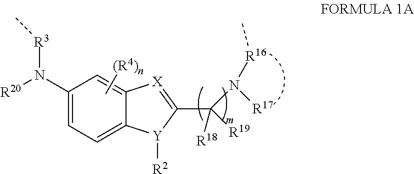

wherein
the “Linker” moiety of the bivalent compound is attached independently to R³ or R¹⁶
X and Y are independently selected from C, O or N;
the definitions of R², R³, R⁴ are the same as for FORMULA 1;
R¹⁶, R¹⁷ is selected from hydrogen, C₁-C₈ alkyl, C₁-C₈ haloalkyl, C₁-C₈ hydroxyalkyl, C₃-C₁₀ cycloalkyl, C₃-C₁₀ heterocycloalkyl, C₆-C₁₀ aryl, C₅-C₁₀ heteroaryl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, C(O)C₆-C₁₀ aryl, C(O)C₅-C₁₀ heteroaryl
or

R¹⁶ and R¹⁷ together with the nitrogen atom to which they connected can independently form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring.

R¹⁸, R¹⁹ are independently selected from hydrogen, halogen, CN, OH, NH₂, optionally substituted C₁-C₈ alkyl, optionally substituted C₃-C₈ cycloalkyl, optionally substituted C₃-C₈ cycloalkoxy, optionally substituted C₁-C₈alkoxyC₁-C₈alkyl, optionally substituted C₁-C₈alkylaminoC₁-C₈alkyl, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;

R²⁰ is selected from hydrogen, optionally substituted C₁-C₈ alkyl, optionally substituted C₃-C₈ cycloalkyl, optionally substituted C₃-C₈ cycloalkoxy, optionally substituted C₃-C₈ heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl.
m, n, are independently selected from 0, 1, 2, 3, and 4;

In an embodiment, ENL ligands include a moiety according to FORMULA 1B, 1C, 1D, 1E

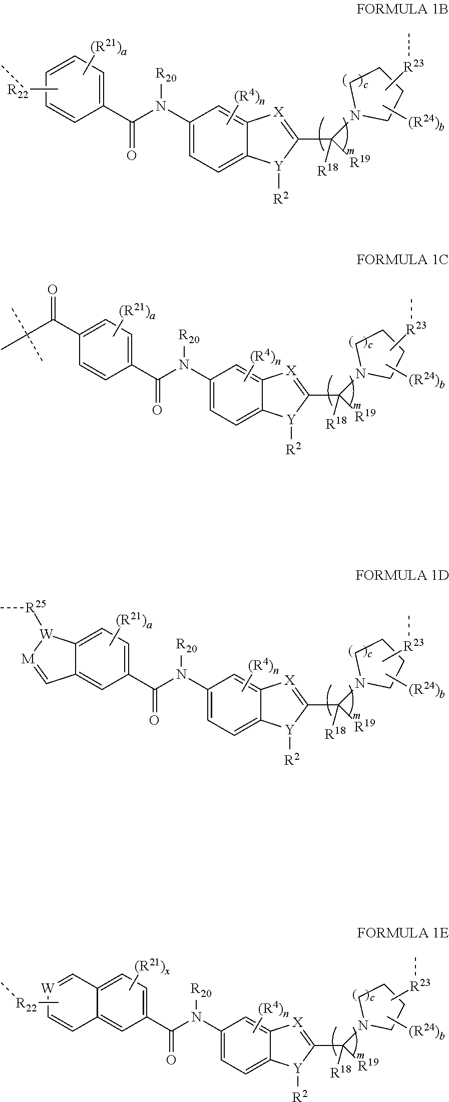

wherein
the “Linker” moiety of the bivalent compound is attached independently to R²², R²³, R²⁵.
X and Y are independently selected from C, O or N;
M and W are independently selected from C or N.
the definitions of R², R⁴, R¹⁸, R¹⁹, R²⁰ are the same as for FORMULA 1A;
   each R²¹ is independently selected from null, hydrogen, halogen, oxo, CN, NO₂, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;

R²² is unsubstituted or optionally substituted with one or more groups selected from halo, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR²⁶R²⁷, C₁-C₈NR²⁶R²⁷, C(O)R²⁶, C(O)OR²⁶, C(O)NR²⁶R²⁷, S(O)R²⁶, S(O)₂R²⁶, S(O)₂NR²⁶R²⁷, NR²⁶C(O)OR²⁷, NR²⁸C(O)R²⁷, NR²⁸S(O)R²⁷, NR²⁸S(O)₂R²⁷.

R²³ is unsubstituted or optionally substituted with one or more groups selected from halo, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR²⁹R³⁰, C(O)R²⁹, C(O)OR²⁹, C(O)NR²⁹R³⁰, S(O)R²⁹, S(O)₂R²⁹, S(O)₂NR²⁹R³⁰, NR³¹C(O)OR²⁹, NR³¹C(O)R²⁹, NR³¹S(O)R²⁹, NR³¹S(O)₂R²⁹
each R²⁴ is independently selected from null, hydrogen, halogen, oxo, CN, NO₂, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;

R²⁵ is unsubstituted or optionally substituted with one or more groups selected from halo, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR³²R³³, C(O)R³², C(O)OR³², C(O)NR³²R³³, S(O)R³², S(O)₂R³², S(O)₂NR³²R³³, NR³⁴C(O)OR³², NR³⁴C(O)R³², NR³⁴S(O)R³², NR³⁴S(O)₂R³².

R²⁶, R²⁷, R²⁸, R²⁹, R³⁰, R³¹ R³², R³³, R³⁴ are independently selected from H, C₁-C₈ alkyl, C₁-C₈ haloalkyl, C₁-C₈ hydroxyalkyl, C₃-C₁₀ cycloalkyl, C₃-C₁₀ heterocyclyl, C(O) C₁-C₈ alkyl, C(O) C₁-C₈ haloalkyl, C(O) C₁-C₈ hydroxyalkyl, C(O) C₃-C₁₀ cycloalkyl, C(O) C₃-C₁₀ heterocyclyl, optionally substituted C₆-C₁₀ aryl or C₅-C₁₀ heteroaryl.

R²⁶ and R²⁷, R²⁷ and R²⁸, R²⁹ and R³⁰, R²⁹ and R³¹, R³² and R³³, R³² and R³⁴, together with the nitrogen atom to which they connected can independently form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring.
m, n, a, b are independently selected from 0, 1, 2, 3, and 4;
c is independently selected from 0, 1, 2, 3, 4, 5 and 6.

In an embodiment, ENL ligands include a moiety according to FORMULA 1F:

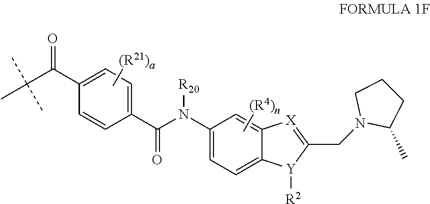

wherein
the “Linker” moiety of the bivalent compound is attached to the carbonyl group indicated with dotted line
the definitions of R², R⁴, R²⁰, R²¹ are the same as for FORMULA 1B;
n, a are independently selected from 0, 1, 2, 3, and 4;

In an embodiment, ENL ligands include a moiety according to FORMULA 2.

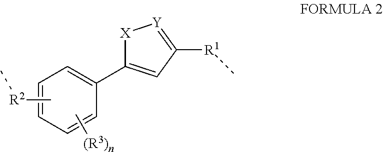

wherein
the “Linker” moiety of the bivalent compound is attached independently to R¹ or R²
X and Y are independently selected from C, O or N;

R¹ is selected from hydrogen, halogen, OR⁴, SR⁴, C₁-C₈ alkylene NR⁴R⁵, C(O)R⁴, C(O)OR⁴, C(S)OR⁴, C(O)NR⁴R⁵, S(O)R⁴, S(O)₂R⁴, S(O)₂NR⁴R⁵, NR⁶C(O)OR⁴, NR⁶C(O)R⁴, NR⁶S(O)R⁴, NR⁶S(O)₂R⁴, or unsubstituted or optionally substituted C₁-C₈ alkyl, C₁-C₈ haloalkyl, C₁-C₈ hydroxyalkyl, C₃-C₁₀ cycloalkyl, C₃-C₁₀ heterocyclyl, or fused C₃-C₁₀ cycloalkyl, C₃-C₁₀ heterocyclyl.

R² is selected from hydrogen, halogen, CN, NO₂, or unsubstituted or optionally substituted C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR⁷R⁸, C(O)R⁷, C(O)OR⁷, C(O)NR⁷R⁸, S(O)R⁷, S(O)₂R⁷, S(O)₂NR⁷R⁸, NR⁹C(O)OR⁷, NR⁹C(O)R⁷, NR⁹S(O)R⁷, NR⁹S(O)₂R⁷, optionally substituted C₆-C₁₀ aryl and optionally substituted C₅-C₁₀ heteroaryl.
each R³ is independently selected from null, hydrogen, halogen, oxo, OH, CN, NO₂, OR¹⁰, SR¹⁰, NR¹⁰R¹¹, OCOR¹⁰, OCO₂R¹⁰, OCONR¹⁰R¹¹, COR¹⁰, CO₂R¹⁰, CONR¹⁰R¹¹, SOR¹⁰, SO₂R¹⁰, SO₂NR¹⁰R¹¹, NR¹²C(O)OR¹⁰, NR¹²C(O)R¹⁰, NR¹²S(O)R¹⁰, NR¹²S(O)₂R¹⁰, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;

wherein

R⁴, R⁵, R⁶, R⁷, R⁸, R⁹, R¹⁰ R¹¹, R¹² are independently selected from H, C₁-C₈ alkyl, C₁-C₈ haloalkyl, C₁-C₈ hydroxyalkyl, C₃-C₁₀ cycloalkyl, C₃-C₁₀ heterocyclyl, C(O) C₁-C₈ alkyl, C(O) C₁-C₈ haloalkyl, C(O) C₁-C₈ hydroxyalkyl, C(O) C₃-C₁₀ cycloalkyl, C(O) C₃-C₁₀ heterocyclyl, optionally substituted C₆-C₁₀ aryl or C₅-C₁₀ heteroaryl.

R⁴ and R⁵, R⁴ and R⁶, R⁷ and R⁸, R⁷ and R⁹, R¹⁰ and R¹¹, R¹⁰ and R¹², together with the nitrogen atom to which they connected can independently form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring.
n is independently selected from 0, 1, 2, 3, 4;

In an embodiment, ENL ligands include a moiety according to FORMULA 2A and 2B.

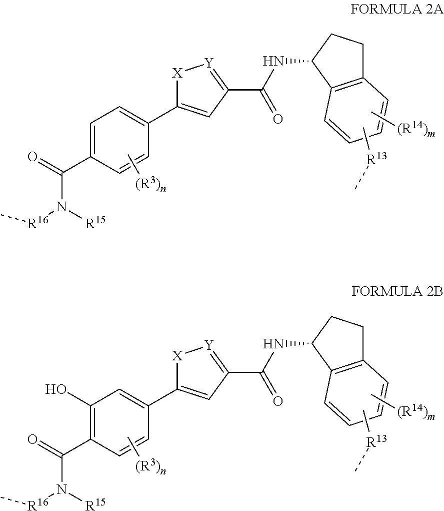

wherein
the “Linker” moiety of the bivalent compound is attached independently to R¹³ or R¹⁶
X and Y are independently selected from C, O or N;
the definitions of R³ is the same as for FORMULA 2;

R¹³ is selected from hydrogen, halogen OR¹⁷, SR¹⁷, C₁-C₈ alkylene NR¹⁷R¹⁸, NR¹⁷R¹⁸, C(O)R¹⁷, C(O)OR¹⁷, C(S)OR¹⁷, C(O)NR¹⁷R¹⁸, S(O)R¹⁷, S(O)₂R¹⁷, S(O)₂NR¹⁷R¹⁸, NR¹⁹C(O)OR¹⁷, NR¹⁹C(O)R¹⁷, NR¹⁹S(O)R¹⁷, NR¹⁹S(O)₂R¹⁷, or unsubstituted or optionally substituted C₁-C₈ alkyl, C₁-C₈ haloalkyl, C₁-C₈ hydroxyalkyl, C₃-C₁₀ cycloalkyl, C₃-C₁₀ heterocyclyl.

each R¹⁴ is independently selected from unsubstituted or optionally substituted with one or more groups selected from hydrogen, halogen, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR²⁰R²¹, C(O)R²⁰, C(O)OR²⁰, C(O)NR²⁰R²¹, S(O)R²⁰, S(O)₂R²⁰, S(O)₂NR²⁰R²¹, NR²²C(O)OR²⁰, NR²²C(O)R²⁰, NR²²S(O)R²⁰, NR²²S(O)₂R²⁰, optionally substituted C₆-C₁₀ aryl and optionally substituted C₅-C₁₀ heteroaryl.

R¹⁵ is selected from hydrogen, optionally substituted C₁-C₈ alkyl, optionally substituted C₃-C₈ cycloalkyl, optionally substituted C₃-C₈ cycloalkoxy, optionally substituted C₃-C₈ heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl.

R¹⁶ is selecy from null, hydrogen, halogen, oxo, CN, NO₂, OR²³, SR²³, NR²³R²⁴, OCOR²³, OCO₂R²³, OCONR²³R²⁴, COR²³, CO₂R²³, CONR²³R²⁴, SOR²³, SO₂R²³, SO₂NR²³R²⁴, NR²⁵C(O)OR²³, NR²⁵C(O)R²³, NR²⁵S(O)R²³, NR²⁵S(O)₂R²³, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;

wherein

R¹⁷, R¹⁸, R¹⁹, R²⁰, R²¹, R²², R²³, R²⁴, R²⁵ are independently selected from H, C₁-C₈ alkyl, C₁-C₈ haloalkyl, C₁-C₈ hydroxyalkyl, C₃-C₁₀ cycloalkyl, C₃-C₁₀ heterocyclyl, C(O) C₁-C₈ alkyl, C(O) C₁-C₈ haloalkyl, C(O) C₁-C₈ hydroxyalkyl, C(O) C₃-C₁₀ cycloalkyl, C(O) C₃-C₁₀ heterocyclyl, optionally substituted C₆-C₁₀ aryl or C₅-C₁₀ heteroaryl.

R¹⁷ and R¹⁸, R¹⁷ and R¹⁹, R²⁰ and R²¹, R²⁰ and R²², R²³ and R²⁴, R²³ and R²⁵, together with the nitrogen atom to which they connected can independently form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring.
m, n is independently selected from 0, 1, 2, 3, 4;
and pharmaceutically acceptable salts thereof.

In an embodiment, ENL ligands include a moiety according to FORMULA 2C.

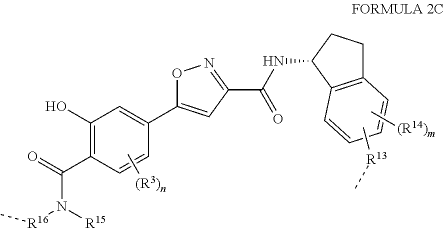

Wherein
the “Linker” moiety of the bivalent compound is attached independently to R¹³ or R¹⁶
the definitions of R³, R¹³, R¹⁴, R¹⁵ an R¹⁶ is the same as for FORMULA 2A and 2B;

In an embodiment, ENL ligands include a moiety according to FORMULA 3.

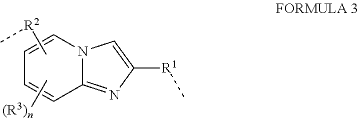

Wherein
the “Linker” moiety of the bivalent compound is attached independently to R¹ or R²
the definitions of R¹, R² and R³ are the same as for FORMULA 2.

In an embodiment, ENL ligands include a moiety according to FORMULA 3A.

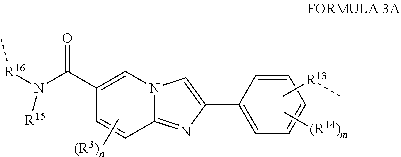

wherein
the “Linker” moiety of the bivalent compound is attached independently to R¹³ or R¹⁶
the definitions of R³, R¹³, R¹⁴, R¹⁵ and R¹⁶ are the same as for FORMULA 2A;
n is selected from 0, 1, 2, 3; and
m is selected from 0, 1, 2, 3, 4.

In an embodiment, (ENL) ligands are selected from the group consisting of:

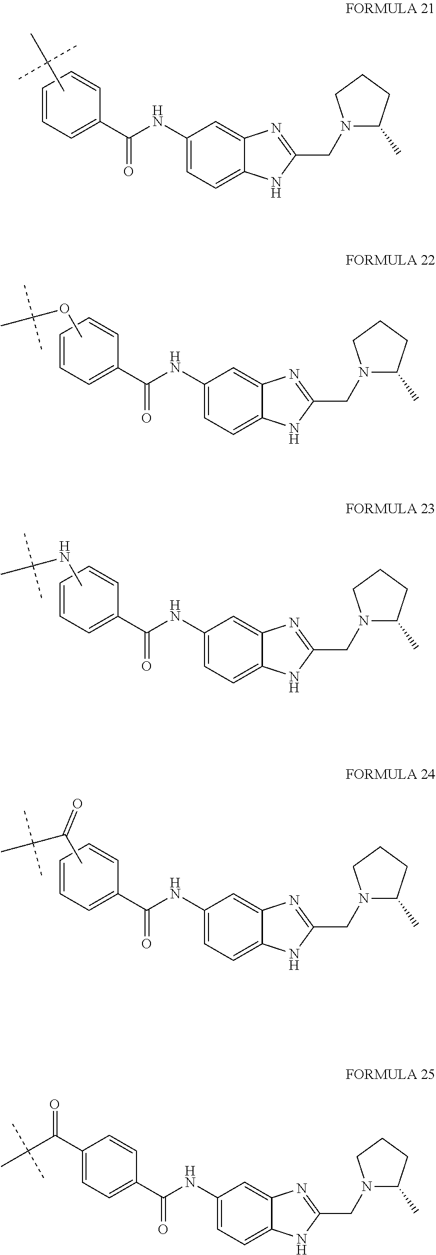

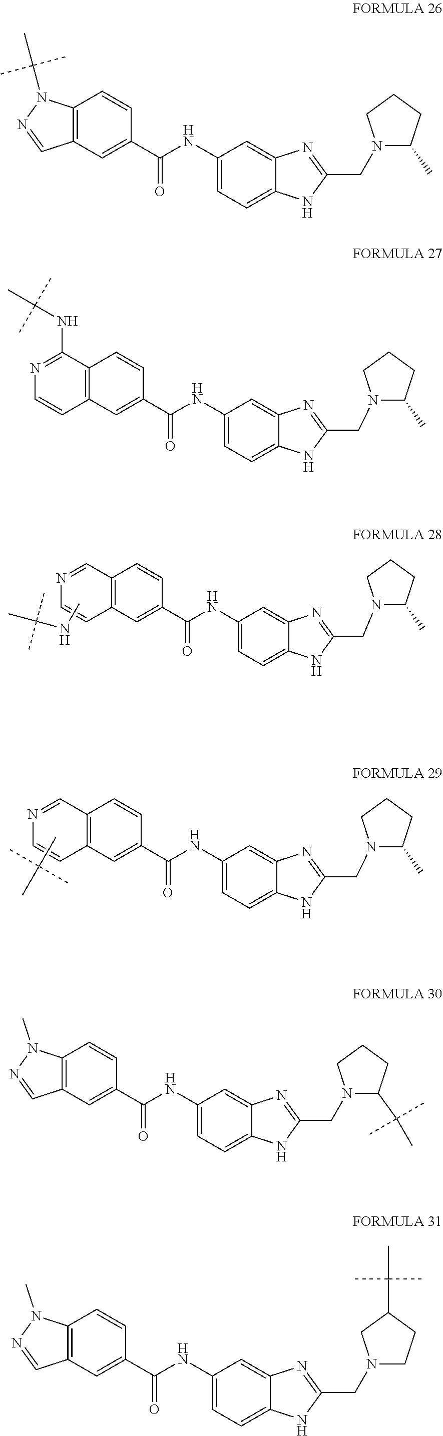

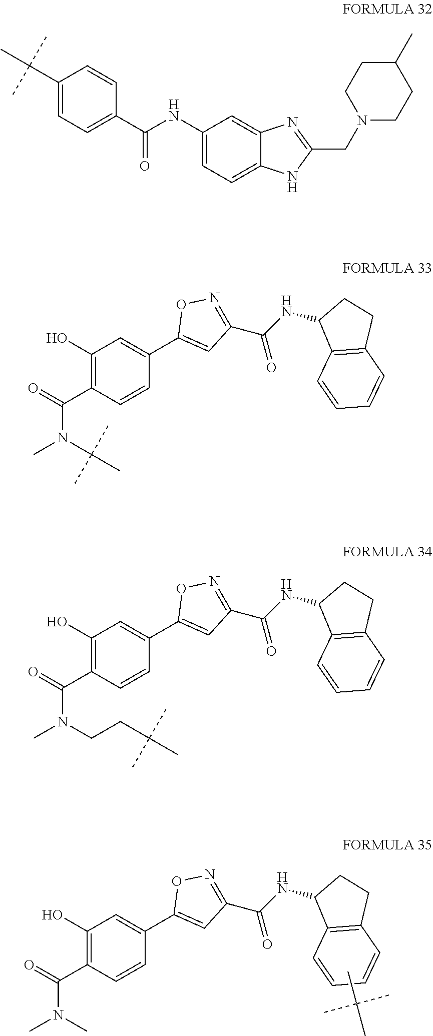

Degradation/Disruption Tags

Degradation/Disruption tags (EL) include, but are not limited to:

In an embodiment, degradation/disruption tags include a moiety according to FORMULAE 4A, 4B, 4C and 4D:

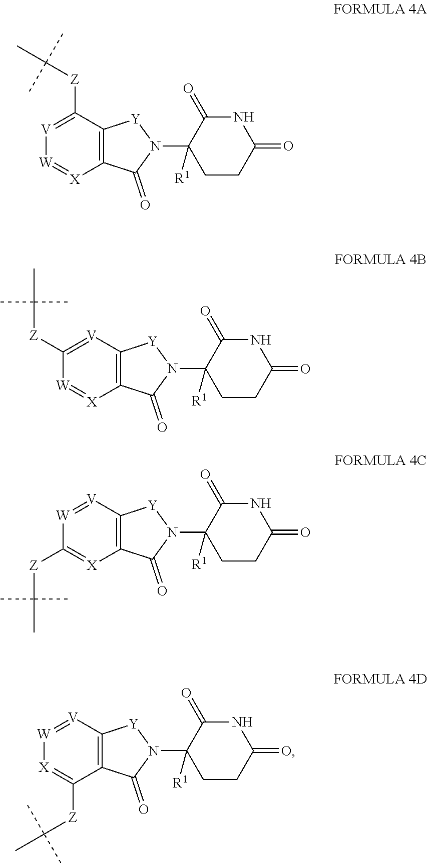

wherein
V, W, and X are independently selected from CR² and N;
Y is selected from CO, CR³R⁴, and N═N;
Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;
R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;
R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and
R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl.

In an embodiment, degradation/disruption tags include a moiety according to one of FORMULAE 4E, 4F, 4G, 4H, and 4I:

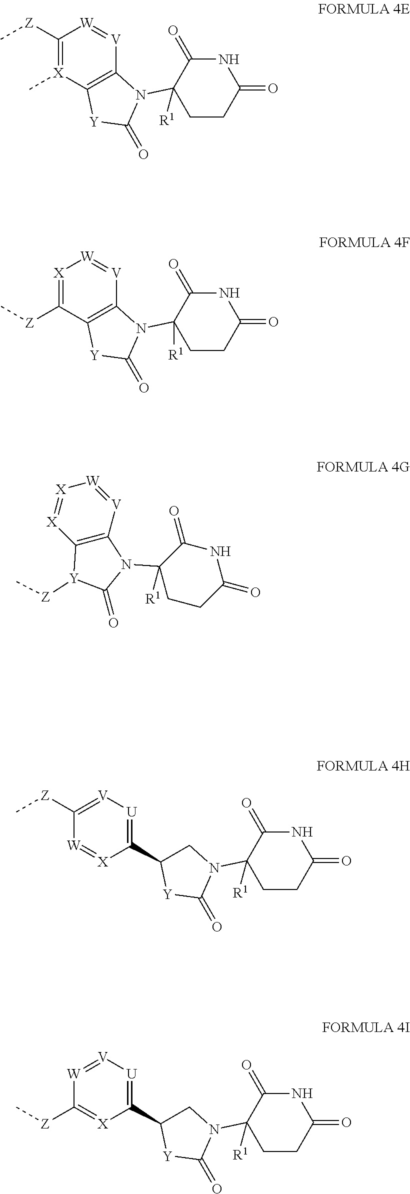

wherein
U, V, W, and X are independently selected from CR² and N;
Y is selected from CR³R⁴, NR³ and O; preferably, Y is selected from CH₂, NH, NCH₃ and O;
Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;
R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;
R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and
R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl; and
   pharmaceutically acceptable salts thereof.

In an embodiment, degradation/disruption tags include a moiety according to FORMULA

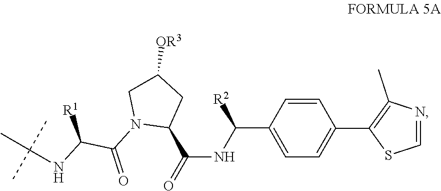

wherein
R¹ and R² are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₇ cycloalkyl, optionally substituted 3-7 membered heterocyclyl, optionally substituted C₂-C₈ alkenyl, and optionally substituted C₂-C₈ alkynyl; and
R³ is hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)C₃-C₇ cycloalkyl, optionally substituted C(O)(3-7 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)OC₃-C₇ cycloalkyl, optionally substituted C(O)O(3-7 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)NC₃-C₇ cycloalkyl, optionally substituted C(O)N(3-7 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂.

In an embodiment, degradation/disruption tags include a moiety according to FORMULAE 5B, 5C, 5D, 5E and 5F:

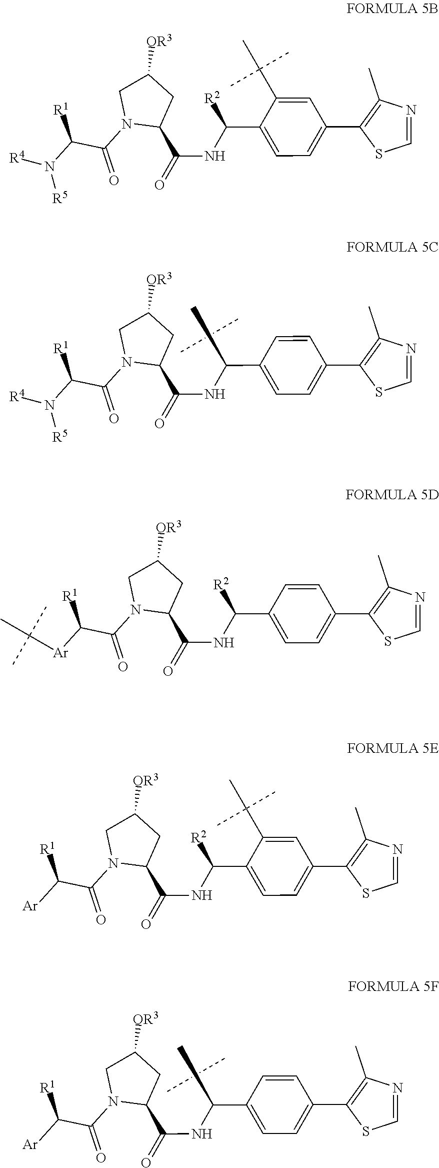

wherein
R¹ and R² are independently selected from hydrogen, halogen, OH, NH₂, CN, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₇ cycloalkyl, optionally substituted 3-7 membered heterocyclyl, optionally substituted C₂-C₈ alkenyl, and optionally substituted C₂-C₈ alkynyl; (preferably, R¹ is selected from iso-propyl or tert-butyl; and R² is selected from hydrogen or methyl);
R³ is hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)C₃-C₇ cycloalkyl, optionally substituted C(O)(3-7 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)OC₃-C₇ cycloalkyl, optionally substituted C(O)O(3-7 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)NC₃-C₇ cycloalkyl, optionally substituted C(O)N(3-7 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂; and
   R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; wherein
   R⁶ and R⁷ are independently selected from hydrogen, optionally substituted C₁-C₈ alkyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; or
   R⁴ and R⁵; R⁶ and R⁷ together with the atom to which they are connected form a 4-8 membered cycloalkyl or heterocyclyl ring;
   Ar is selected from aryl and heteroaryl, each of which is optionally substituted with one or more substituents independently selected from F, Cl, CN, NO₂, OR⁸, NR⁸R⁹, COR⁸, CO₂R⁸, CONR⁸R⁹, SOR⁸, SO₂R⁸, SO₂NR⁹R¹⁰, NR⁹COR¹⁰, NR⁸C(O)NR⁹R¹⁰, NR⁹SOR¹⁰, NR⁹SO₂R¹⁰, optionally substituted C₁-C₆ alkyl, optionally substituted C₁-C₆ alkoxyalkyl, optionally substituted C₁-C₆ haloalkyl, optionally substituted C₁-C₆ hydroxyalkyl, optionally substituted C₁-C₆alkylaminoC₁-C₆alkyl, optionally substituted C₃-C₇ cycloalkyl, optionally substituted 3-7 membered heterocyclyl, optionally substituted C₂-C₆ alkenyl, optionally substituted C₂-C₆ alkynyl, optionally substituted aryl, and optionally substituted C₄-C₅ heteroaryl; wherein
   R⁸, R⁹, and R¹⁰ are independently selected from null, hydrogen, optionally substituted C₁-C₆ alkyl, optionally substituted C₂-C₆ alkenyl, optionally substituted C₂-C₆ alkynyl, optionally substituted C₃-C₇ cycloalkyl, optionally substituted 3-7 membered heterocyclyl, optionally substituted aryl, and optionally substituted heteroaryl; or
   R⁸ and R⁹; R⁹ and R¹⁰ together with the atom to which they are connected form a 4-8 membered cycloalkyl or heterocyclyl ring; and
pharmaceutically acceptable salts thereof.

In an embodiment, degradation/disruption tags include a moiety according to FORMULA 5A:

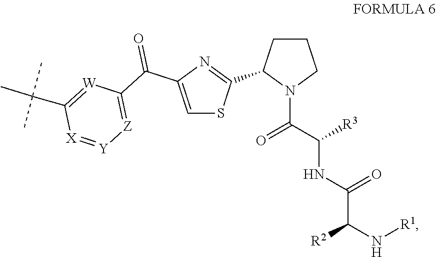

wherein
V, W, X, and Z are independently selected from CR⁴ and N;

R¹, R², R³, and R⁴ are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl.

In an embodiment, degradation/disruption tags include a moiety according to FORMULA 5B:

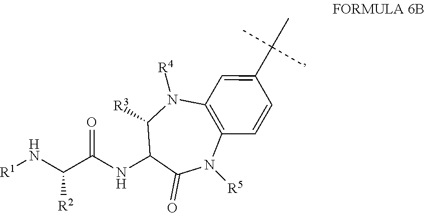

wherein
R¹, R², and R³ are independently selected from hydrogen, halogene, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl;
   R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted aryl-C₁-C₈alkyl, optionally substituted 3-8 membered cycloalkyl, optionally substituted 3-8 membered heterocyclyl, optionally substituted aryl, and optionally substituted heteroaryl; wherein
   R⁶ and R⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; or
   R⁶ and R⁷ together with the atom to which they are connected form a 4-8 membered cycloalkyl or heterocyclyl ring; and
pharmaceutically acceptable salts thereof.

In an embodiment, degradation/disruption tags are selected from the group consisting of:

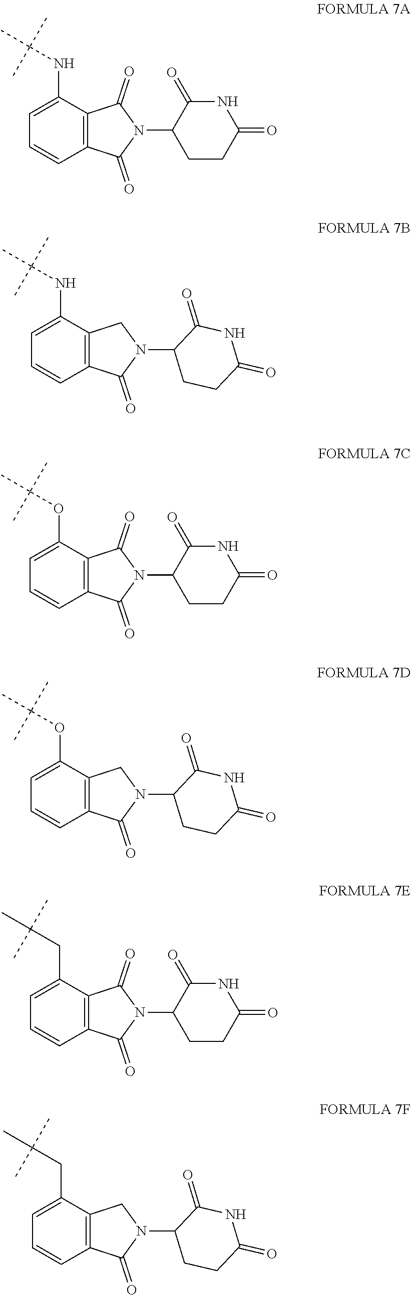

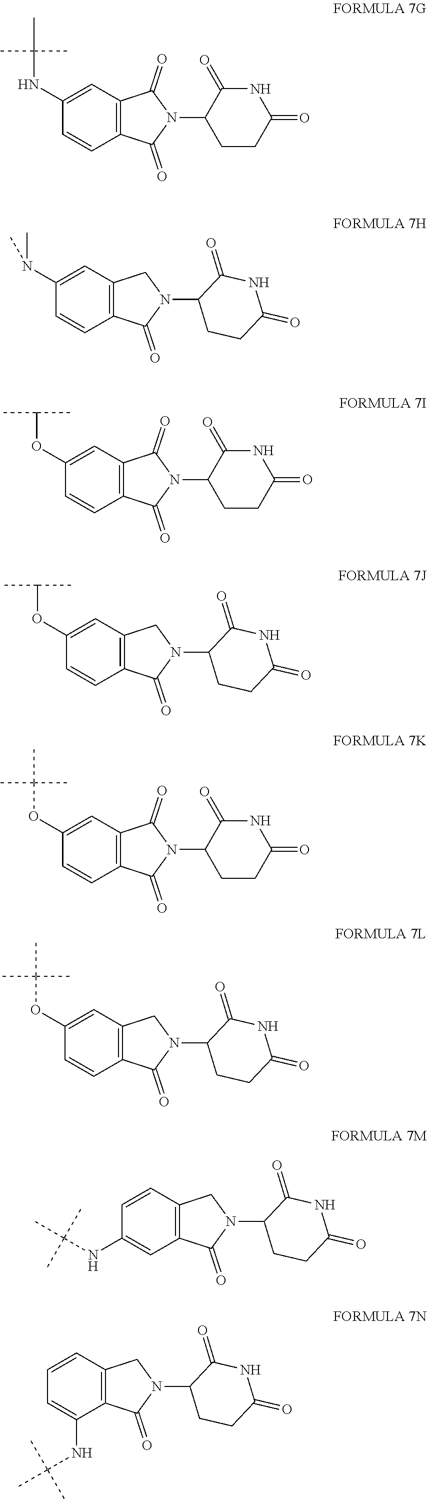

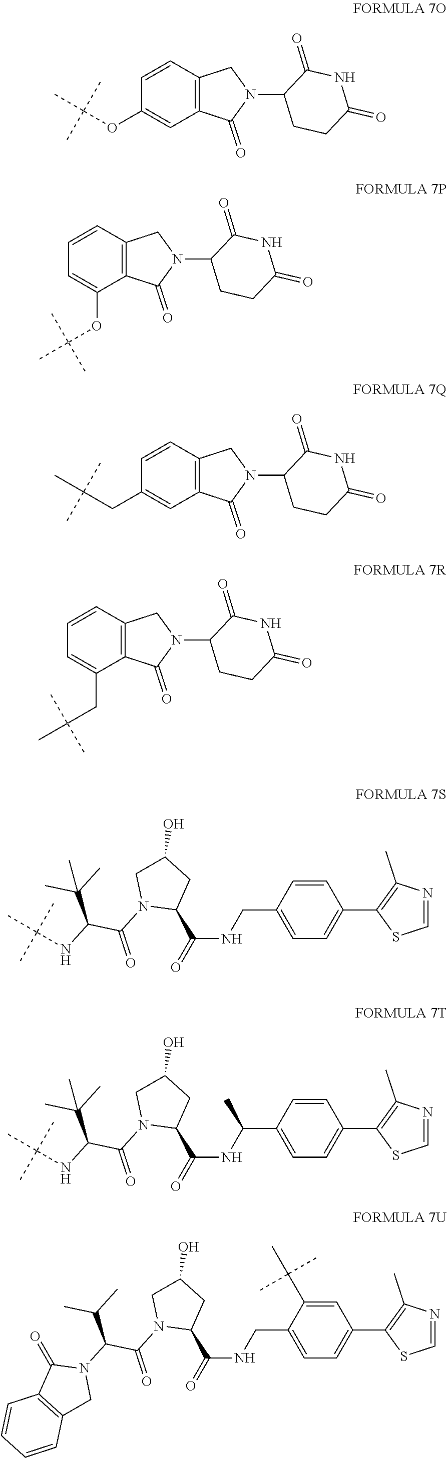

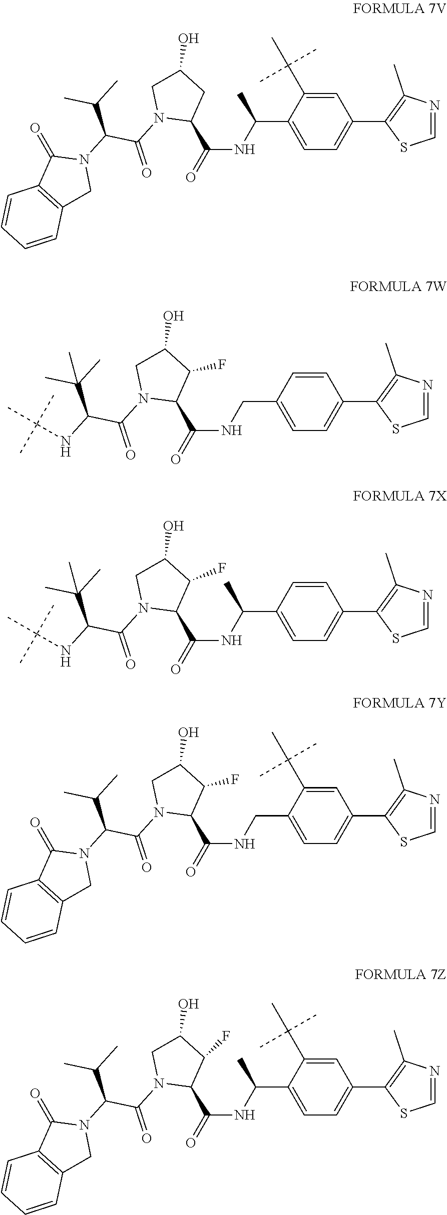

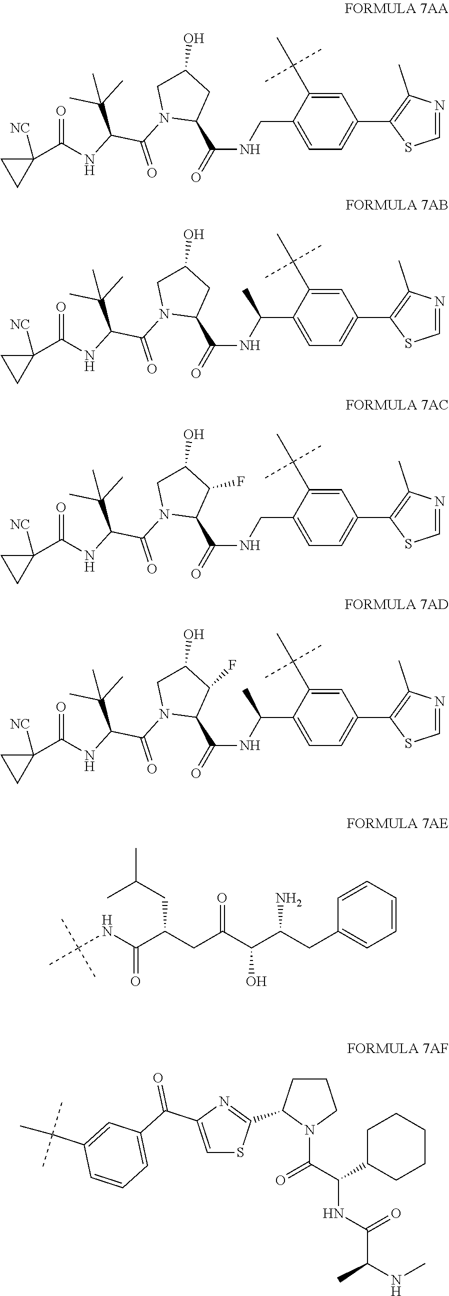

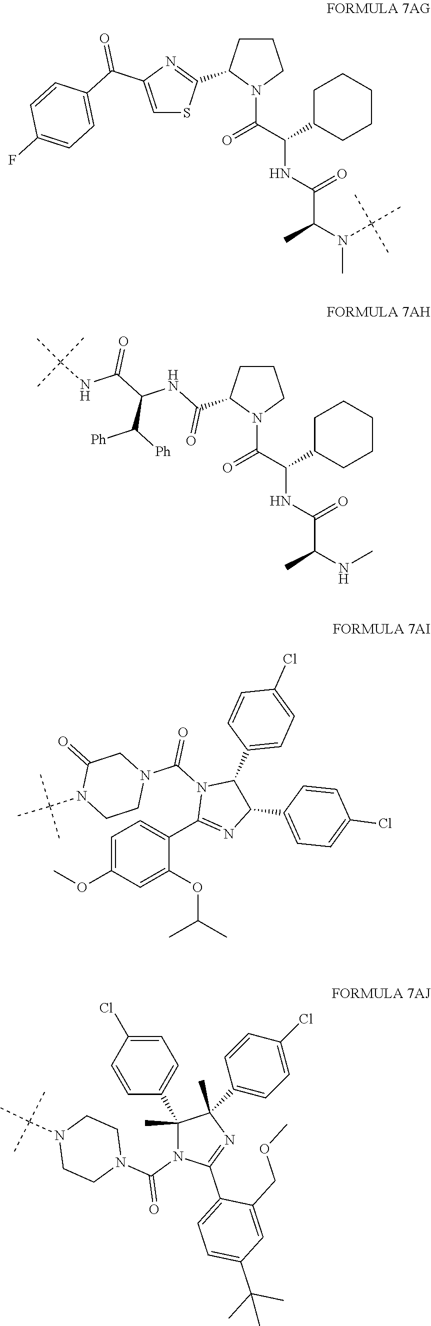

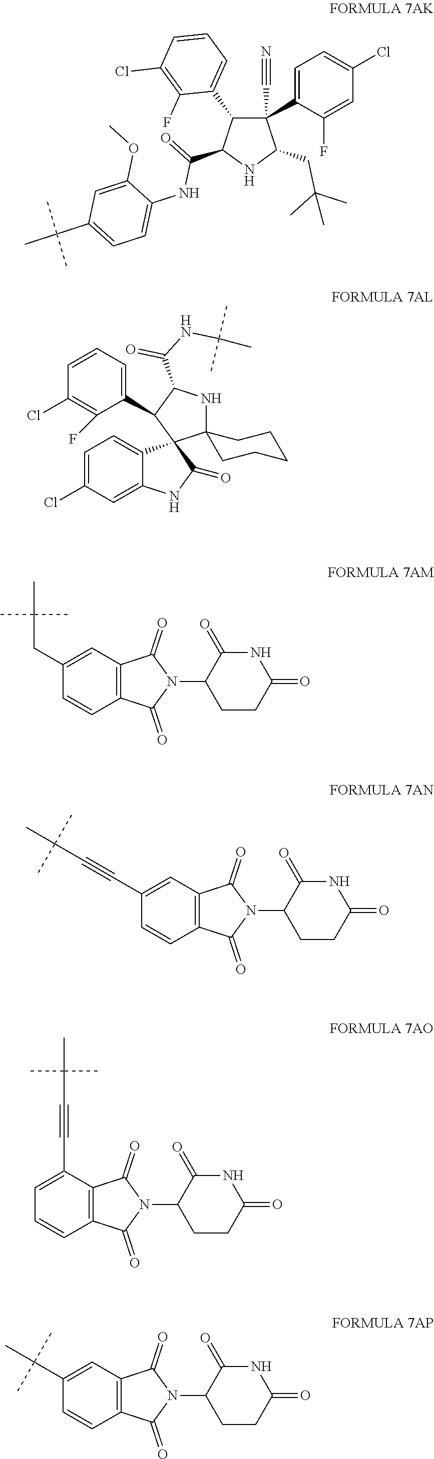

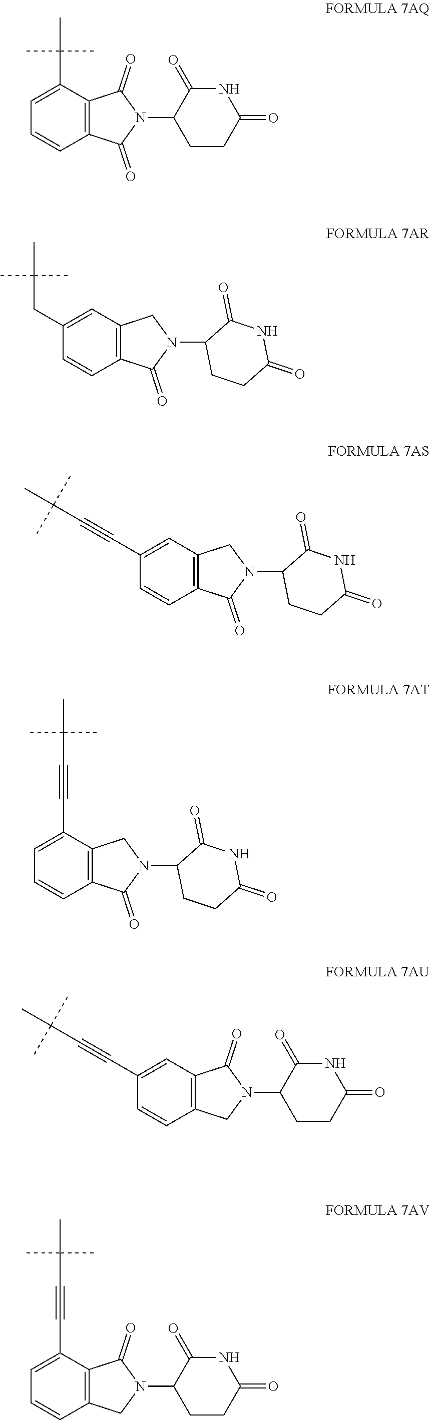

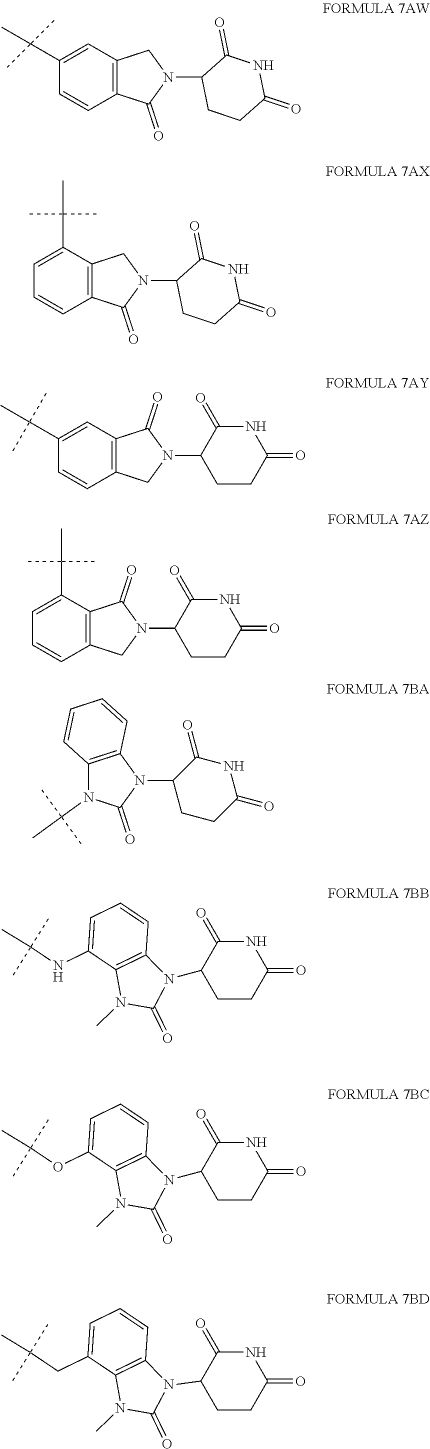

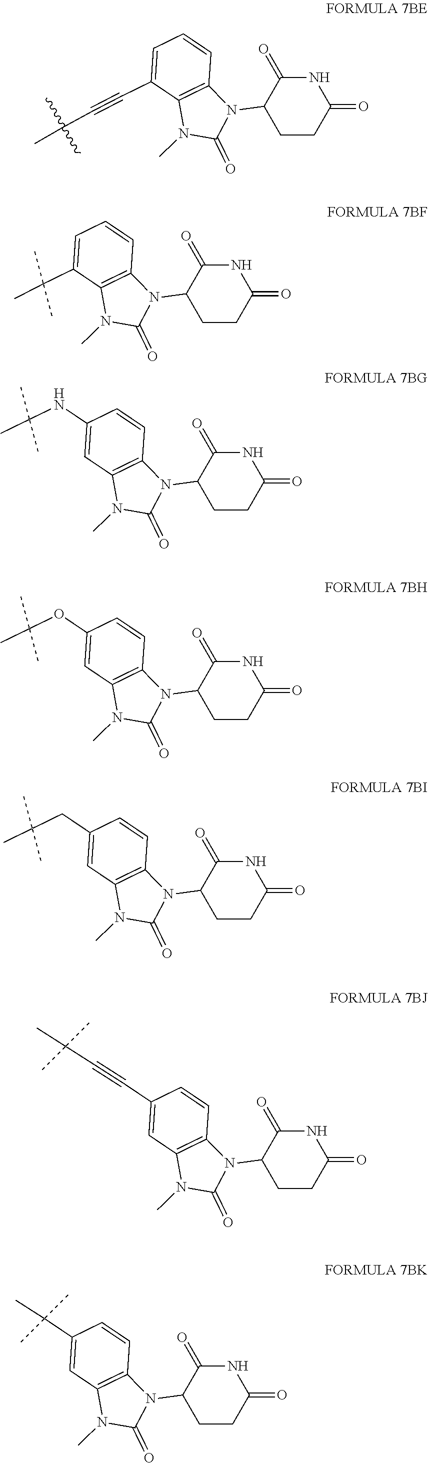

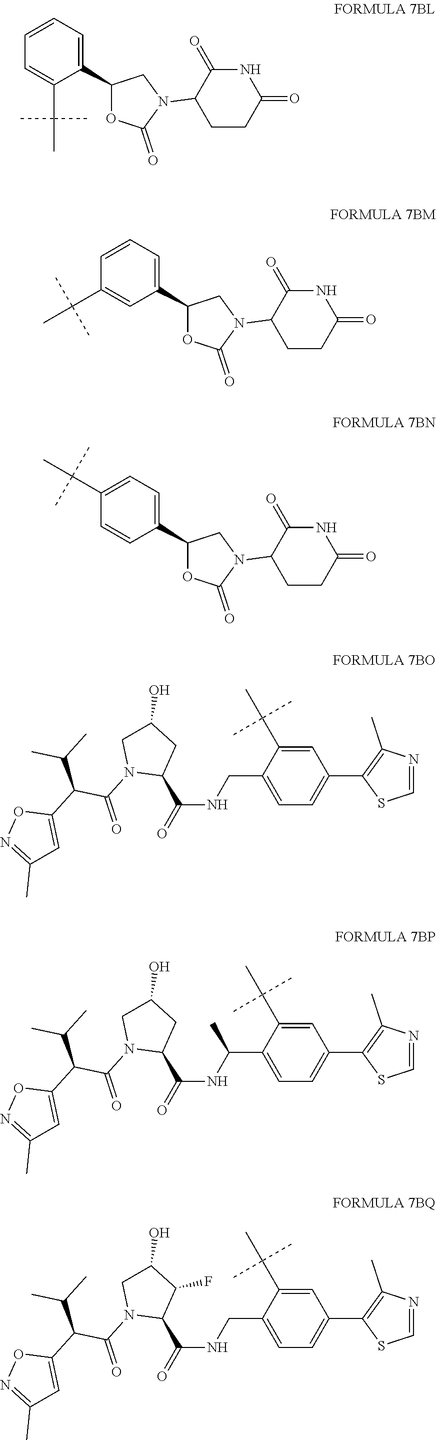

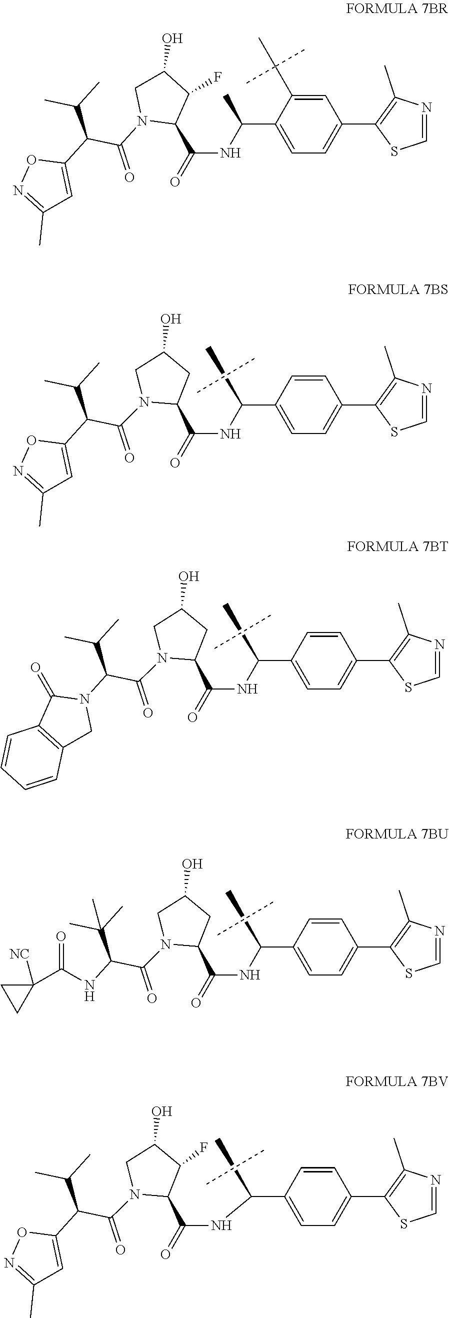

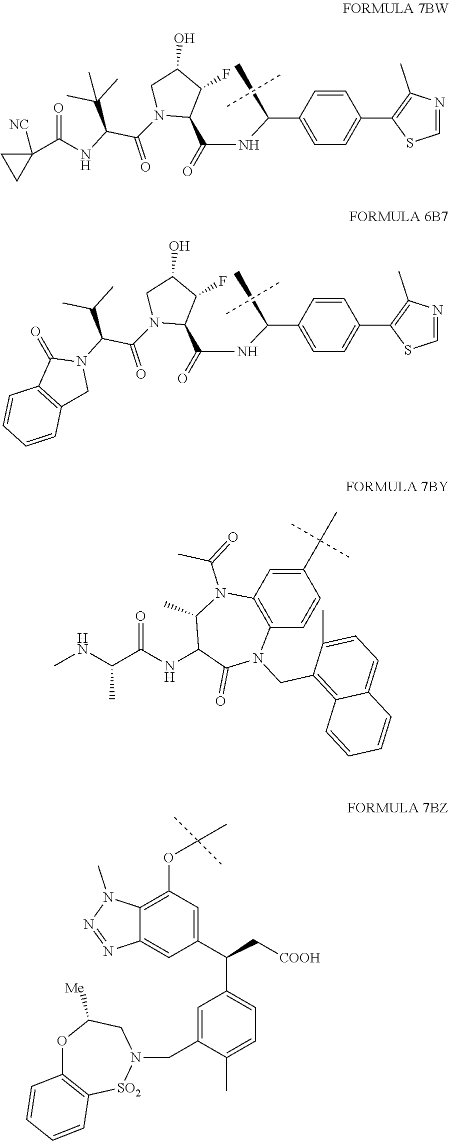

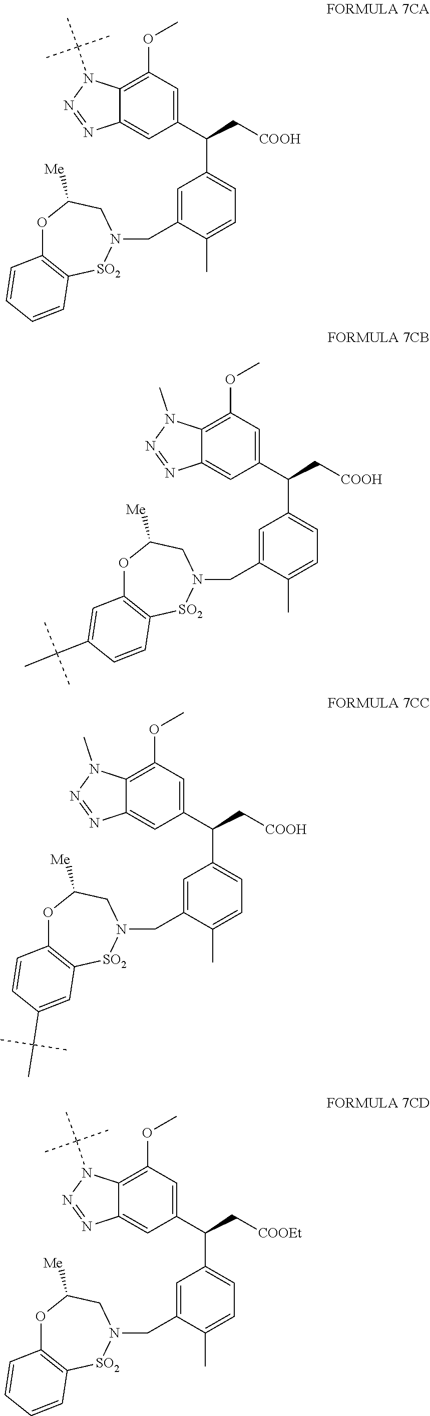

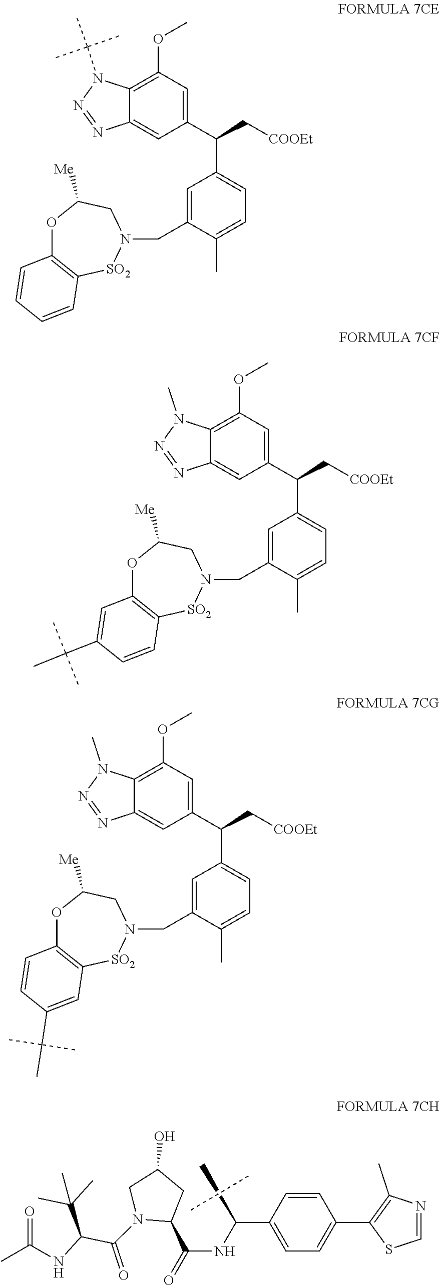

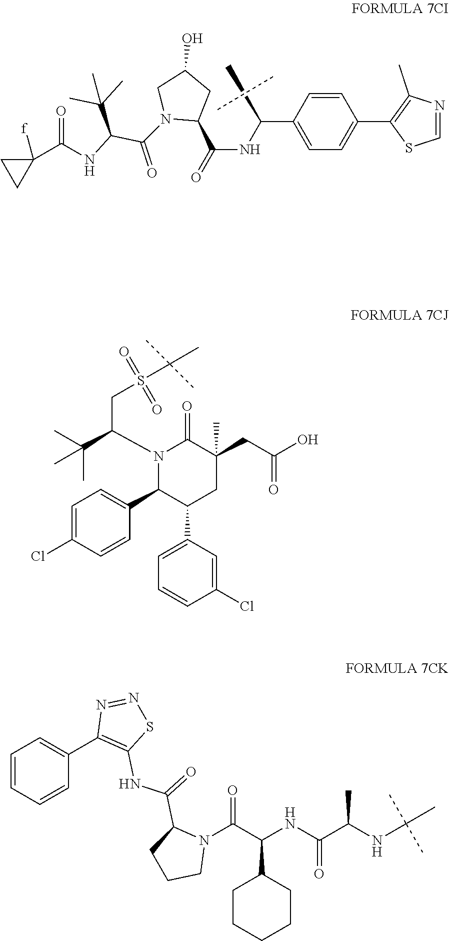

and pharmaceutically acceptable salts thereof.

### Linkers

In any of the above-described compounds, the ENL ligand can be conjugated to the degradation/disruption tag through a linker. The linker can include, e.g., acyclic or cyclic saturated or unsaturated carbon, ethylene glycol, amide, amino, ether, urea, carbamate, aromatic, heteroaromatic, heterocyclic, and/or carbonyl containing groups with different lengths.

In an embodiment, the linker is a moiety according to FORMULA 8:

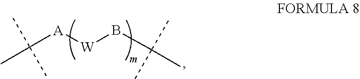

wherein
A, W, and B, at each occurrence, are independently selected from null, CO, CO₂, C(O)NR¹, C(S)NR¹, O, S, SO, SO₂, SO₂NR¹, NR¹, NR¹CO, NR¹CONR², NR¹C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R¹ and R² are independently selected from hydrogen, optionally substituted C₁-C₈ alkyl, optionally substituted C₃-C₈ cycloalkyl, optionally substituted 3-8 membered cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈alkylaminoC₁-C₈alkyl; and
m is 0 to 15.

In an embodiment, the linker is a moiety according to FORMULA 8A:

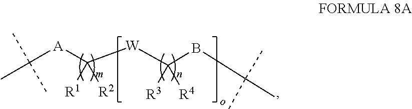

wherein
R¹, R², R³, and R⁴, at each occurrence, are independently selected from hydrogen, halogen, CN, OH, NH₂, optionally substituted C₁-C₈ alkyl, optionally substituted C₃-C₈ cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
A, W, and B, at each occurrence, are independently selected from null, CO, CO₂, C(O)NR⁵, C(S)NR⁵, O, S, SO, SO₂, SO₂NR⁵, NR⁵, NR⁵CO, NR⁵CONR⁶, NR⁵C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
   R⁵ and R⁶ are independently selected from hydrogen, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈alkylaminoC₁-C₈alkyl;
m is 0 to 15;
n, at each occurrence, is 0 to 15;
o is 0 to 15.

In an embodiment, the linker is a moiety according to FORMULA 8B:

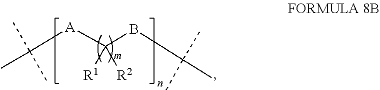

wherein
R¹ and R², at each occurrence, are independently selected from hydrogen, halogen, CN, OH, NH₂, and optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, or C₁-C₈alkylaminoC₁-C₈alkyl;
A and B, at each occurrence, are independently selected from null, CO, CO₂, C(O)NR³, C(S)NR³, O, S, SO, SO₂, SO₂NR³, NR³, NR³CO, NR³CONR⁴, NR³C(S), and optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, or C₃-C₁₃ spiro heterocyclyl; wherein
R³ and R⁴ are independently selected from hydrogen, and optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, or C₁-C₈alkylaminoC₁-C₈alkyl;
each m is 0 to 15; and
n is 0 to 15.

In an embodiment, the linker is a moiety according to FORMULA 8C:

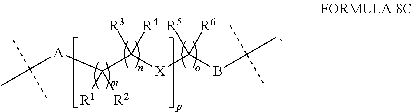

wherein
X is selected from 0, NH, and NR⁷;
R¹, R², R³, R⁴, R⁵, and R⁶, at each occurrence, are independently selected from hydrogen, halogen, CN, OH, NH₂, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH—CH₂—NH—CH₂, CH₂—NH—CH₂, CO₂, C(O)NR⁷, C(S)NR⁷, O, S, SO, SO₂, SO₂NR⁷, NR⁷, NR⁷CO, NR⁷CONR⁸, NR⁷C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R⁷ and R⁸ are independently selected from hydrogen, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
m, at each occurrence, is 0 to 15;
n, at each occurrence, is 0 to 15;
o is 0 to 15; and
p is 0 to 15; and
pharmaceutically acceptable salts thereof.

In an embodiment, the linker is selected from the group consisting of a ring selected from the group consisting of a 3 to 13 membered ring; a 3 to 13 membered fused ring; a 3 to 13 membered bridged ring; and a 3 to 13 membered spiro ring; and pharmaceutically acceptable salts thereof.

In an embodiment, the linker is a moiety according to one of FORMULAE C1, C2, C3, C4 and C5.

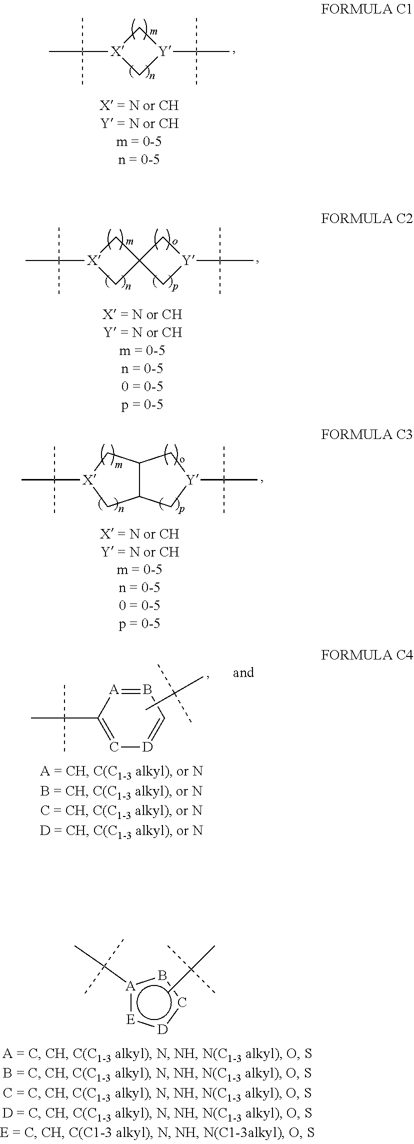

FORMULA C5; and pharmaceutically acceptable salts thereof.

In an embodiment, the bivalent compound according to the present invention is selected from the group consisting of:
LQ076-46, LQ076-47, LQ076-48, LQ076-49, LQ076-50, LQ076-51, LQ076-52, LQ076-53, LQ076-54, LQ076-55, LQ076-56, LQ076-57, LQ076-58, LQ076-59, LQ076-60, LQ076-61, LQ076-62, LQ076-63, LQ076-64, LQ076-65, LQ076-66, LQ076-67, LQ076-68, LQ076-69, LQ076-70, LQ076-71, LQ076-72, LQ076-73, LQ076-74, LQ076-75, LQ076-76, LQ076-77, LQ076-78, LQ076-79, LQ076-80, LQ076-81, LQ076-82, LQ076-83, LQ076-84, LQ076-85, LQ076-86, LQ076-87, LQ076-88, LQ076-89, LQ076-90, LQ076-91, LQ076-92, LQ076-93, LQ076-94, LQ076-95, LQ076-96, LQ076-97, LQ076-98, LQ076-99, LQ076-100, LQ076-101, LQ076-102, LQ076-103, LQ076-104, LQ076-105, LQ076-106, LQ076-107, LQ076-108, LQ076-109, LQ076-110, LQ076-111, LQ076-112, LQ076-113, LQ076-114, LQ076-115, LQ076-116, LQ076-117, LQ076-118, LQ076-119, LQ076-120, LQ076-121, LQ076-122, LQ076-123, LQ076-124, LQ076-125, LQ076-126, LQ076-127, LQ076-128, LQ076-129, LQ076-130, LQ076-131, LQ076-132, LQ076-133, LQ076-134, LQ076-135, LQ076-136, LQ076-137, LQ076-138, LQ076-139, LQ076-140, LQ076-141, LQ076-142, LQ076-143, LQ076-144, LQ076-145, LQ076-146, LQ076-147, LQ076-148, LQ076-149, LQ076-150, LQ076-151, LQ076-152, LQ076-153, LQ076-154, LQ076-155, LQ076-156, LQ076-157, LQ076-158, LQ076-159, LQ076-160, LQ076-161, LQ076-162, LQ076-163, LQ081-100, LQ081-101, LQ081-102, LQ081-103, LQ081-104, LQ081-105, LQ081-108, LQ081-109, LQ081-122, LQ081-132, LQ081-133, LQ081-146, LQ081-147, LQ081-150, LQ086-31, LQ086-32, LQ086-33, LQ086-34, LQ086-35, LQ086-36, LQ086-38, LQ086-40, LQ086-41, LQ086-76, LQ086-76Na, LQ108-6, LQ108-7, LQ108-8, LQ108-9, LQ108-10, LQ108-11, LQ108-12, LQ108-146, LQ108-147, LQ108-148, LQ108-149, LQ108-150, LQ108-151, LQ108-152, LQ108-153, LQ108-154, LQ108-155, LQ108-156, LQ108-157, LQ118-23, LQ118-24, LQ118-25, LQ108-58, LQ108-60, LQ108-61, LQ108-62, LQ108-63, LQ108-64, LQ108-65, LQ108-66, LQ108-67, LQ108-68, LQ108-69, LQ108-70, LQ108-71, LQ108-72, LQ108-73, LQ108-74, LQ108-75, LQ126-46, LQ126-49, LQ126-50, LQ126-51, LQ126-52, LQ126-53, LQ126-54, LQ126-55, LQ126-56, LQ126-57, LQ126-58, LQ126-59, LQ126-60, LQ126-61, LQ126-62, LQ126-63, LQ126-77, LQ126-78, LQ126-79, LQ126-80, LQ126-81, LQ126-82, LQ126-83, LQ126-84, LQ126-85, LQ126-86, LQ126-87, LQ126-89, LQ126-90, LQ126-91, LQ126-92, LQ126-93, LQ126-94, LQ126-95, LQ126-96, LQ126-97, LQ126-98, LQ126-99, LQ126-100, LQ126-101, LQ126-102, LQ126-103, LQ126-104, LQ126-105, LQ126-106, LQ126-107, LQ126-108, LQ126-109, LQ126-110, LQ126-112, LQ126-113, LQ126-114, LQ126-115, LQ126-116, LQ126-117, LQ126-118, LQ126-120, LQ126-121, LQ126-122, LQ126-123, LQ126-124, LQ126-125, LQ126-126, LQ126-127, LQ126-128, LQ126-130, LQ126-168, LQ126-170, LQ126-171, LQ126-172, LQ126-173, LQ126-174, LQ126-175, LQ126-176, LQ126-177, LQ126-178, LQ126-180, LQ126-181, LQ126-182, LQ126-183, LQ126-184, LQ126-185, LQ126-186, LQ141-1, LQ141-2, LQ141-3, LQ141-4, LQ141-5, LQ141-6, LQ141-7, LQ141-8, LQ141-9, LQ141-10, LQ141-11, LQ141-12, LQ141-13, LQ141-14, LQ141-15, LQ141-16, LQ141-17, LQ141-18, LQ141-19, LQ141-20, LQ141-21, LQ141-22, LQ141-24, LQ141-26, LQ141-27, LQ141-28, LQ141-29, LQ141-33, LQ141-36, LQ141-37, LQ141-38, LQ141-39, LQ141-42, LQ141-43, LQ141-44, LQ141-45, LQ141-46, LQ141-47, LQ141-48, LQ141-49, LQ141-52 and LQ141-57.

In one embodiment, preferred compounds according to the present invention include:
a. N¹-(11-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-11-oxoundecyl)-N⁴-(2-(((S)-2-methylpyrrolidin-1-yl)methyl)-1H-benzo[d]imidazol-5-yl)terephthalamide (LQ076-122);
b. N¹-(11-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-11-oxoundecyl)-N⁴-(2-(((S)-2-methylpyrrolidin-1-yl)methyl)-1H-benzo[d]imidazol-5-yl)terephthalamide (LQ081-108); and
c. N¹-(12-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-12-oxododecyl)-N⁴-(2-(((S)-2-methylpyrrolidin-1-yl)methyl)-1H-benzo[d]imidazol-5-yl)terephthalamide (LQ081-109).

In one embodiment, preferred compounds according to the present invention also include:
a. 5-(4-(dimethylcarbamoyl)-3-hydroxyphenyl)-N—((R)-6-((6-((S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanamido)hexyl)carbamoyl)-2,3-dihydro-1H-inden-1-yl)isoxazole-3-carboxamide (LQ108-69);
b. 5-(4-(dimethylcarbamoyl)-3-hydroxyphenyl)-N—((R)-6-((7-((S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanamido)heptyl)carbamoyl)-2,3-dihydro-1H-inden-1-yl)isoxazole-3-carboxamide (LQ108-70);
c. 5-(4-(dimethylcarbamoyl)-3-hydroxyphenyl)-N—((R)-6-((8-((S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanamido)octyl)carbamoyl)-2,3-dihydro-1H-inden-1-yl)isoxazole-3-carboxamide (LQ108-71);
d. 5-(4-(dimethylcarbamoyl)-3-hydroxyphenyl)-N—((R)-6-((9-((S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanamido)nonyl)carbamoyl)-2,3-dihydro-1H-inden-1-yl)isoxazole-3-carboxamide (LQ108-72);
e. 5-(4-(dimethylcarbamoyl)-3-hydroxyphenyl)-N—((R)-6-((10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)carbamoyl)-2,3-dihydro-1H-inden-1-yl)isoxazole-3-carboxamide (LQ126-62);
f. 5-(4-(dimethylcarbamoyl)-3-hydroxyphenyl)-N—((R)-6-((11-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-11-oxoundecyl)carbamoyl)-2,3-dihydro-1H-inden-1-yl)isoxazole-3-carboxamide (LQ126-63);
g. 5-(4-(dimethylcarbamoyl)-3-hydroxyphenyl)-N-((1R)-6-((6-(2-((2-(2,6-dioxopiperidin-3-yl)-1,3-dioxoisoindolin-4-yl)oxy)acetamido)hexyl)carbamoyl)-2,3-dihydro-1H-inden-1-yl)isoxazole-3-carboxamide (LQ126-81); and
h. 5-(4-(dimethylcarbamoyl)-3-hydroxyphenyl)-N-((1R)-6-((7-(2-((2-(2,6-dioxopiperidin-3-yl)-1,3-dioxoisoindolin-4-yl)oxy)acetamido)heptyl)carbamoyl)-2,3-dihydro-1H-inden-1-yl)isoxazole-3-carboxamide (LQ126-82).

In some aspects, this disclosure provides a method of treating the ENL-mediated diseases, the method including administering to a subject in need thereof with an ENL-mediated disease one or more bivalent compounds including an ENL ligand conjugated to a degradation/disruption tag. The ENL-mediated diseases may be a disease resulting from ENL amplification. The ENL-mediated diseases can have elevated ENL enzymatic activity relative to a wild-type tissue of the same species and tissue type. Non-limiting examples of ENL-mediated diseases or diseases whose clinical symptoms could be treated by ENL degraders/disruptors-mediated therapy include: all solid and liquid cancer, chronic infections that produce exhausted immune response, infection-mediated immune suppression, age-related decline in immune response, age-related decline in cognitive function and infertility.

In any of the above-described methods, the bivalent compounds can be LQ076-46, LQ076-47, LQ076-48, LQ076-49, LQ076-50, LQ076-51, LQ076-52, LQ076-53, LQ076-54, LQ076-55, LQ076-56, LQ076-57, LQ076-58, LQ076-59, LQ076-60, LQ076-61, LQ076-62, LQ076-63, LQ076-64, LQ076-65, LQ076-66, LQ076-67, LQ076-68, LQ076-69, LQ076-70, LQ076-71, LQ076-72, LQ076-73, LQ076-74, LQ076-75, LQ076-76, LQ076-77, LQ076-78, LQ076-79, LQ076-80, LQ076-81, LQ076-82, LQ076-83, LQ076-84, LQ076-85, LQ076-86, LQ076-87, LQ076-88, LQ076-89, LQ076-90, LQ076-91, LQ076-92, LQ076-93, LQ076-94, LQ076-95, LQ076-96, LQ076-97, LQ076-98, LQ076-99, LQ076-100, LQ076-101, LQ076-102, LQ076-103, LQ076-104, LQ076-105, LQ076-106, LQ076-107, LQ076-108, LQ076-109, LQ076-110, LQ076-111, LQ076-112, LQ076-113, LQ076-114, LQ076-115, LQ076-116, LQ076-117, LQ076-118, LQ076-119, LQ076-120, LQ076-121, LQ076-122, LQ076-123, LQ076-124, LQ076-125, LQ076-126, LQ076-127, LQ076-128, LQ076-129, LQ076-130, LQ076-131, LQ076-132, LQ076-133, LQ076-134, LQ076-135, LQ076-136, LQ076-137, LQ076-138, LQ076-139, LQ076-140, LQ076-141, LQ076-142, LQ076-143, LQ076-144, LQ076-145, LQ076-146, LQ076-147, LQ076-148, LQ076-149, LQ076-150, LQ076-151, LQ076-152, LQ076-153, LQ076-154, LQ076-155, LQ076-156, LQ076-157, LQ076-158, LQ076-159, LQ076-160, LQ076-161, LQ076-162, LQ076-163, LQ081-100, LQ081-101, LQ081-102, LQ081-103, LQ081-104, LQ081-105, LQ081-108, LQ081-109, LQ081-122, LQ081-132, LQ081-133, LQ081-146, LQ081-147, LQ081-150, LQ086-31, LQ086-32, LQ086-33, LQ086-34, LQ086-35, LQ086-36, LQ086-38, LQ086-40, LQ086-41, LQ086-76, LQ086-76Na, LQ108-6, LQ108-7, LQ108-8, LQ108-9, LQ108-10, LQ108-11, LQ108-12, LQ108-146, LQ108-147, LQ108-148, LQ108-149, LQ108-150, LQ108-151, LQ108-152, LQ108-153, LQ108-154, LQ108-155, LQ108-156, LQ108-157, LQ118-23, LQ118-24, LQ118-25, LQ108-58, LQ108-60, LQ108-61, LQ108-62, LQ108-63, LQ108-64, LQ108-65, LQ108-66, LQ108-67, LQ108-68, LQ108-69, LQ108-70, LQ108-71, LQ108-72, LQ108-73, LQ108-74, LQ108-75, LQ126-46, LQ126-49, LQ126-50, LQ126-51, LQ126-52, LQ126-53, LQ126-54, LQ126-55, LQ126-56, LQ126-57, LQ126-58, LQ126-59, LQ126-60, LQ126-61, LQ126-62, LQ126-63, LQ126-77, LQ126-78, LQ126-79, LQ126-80, LQ126-81, LQ126-82, LQ126-83, LQ126-84, LQ126-85, LQ126-86, LQ126-87, LQ126-89, LQ126-90, LQ126-91, LQ126-92, LQ126-93, LQ126-94, LQ126-95, LQ126-96, LQ126-97, LQ126-98, LQ126-99, LQ126-100, LQ126-101, LQ126-102, LQ126-103, LQ126-104, LQ126-105, LQ126-106, LQ126-107, LQ126-108, LQ126-109, LQ126-110, LQ126-112, LQ126-113, LQ126-114, LQ126-115, LQ126-116, LQ126-117, LQ126-118, LQ126-120, LQ126-121, LQ126-122, LQ126-123, LQ126-124, LQ126-125, LQ126-126, LQ126-127, LQ126-128, LQ126-130, LQ126-168, LQ126-170, LQ126-171, LQ126-172, LQ126-173, LQ126-174, LQ126-175, LQ126-176, LQ126-177, LQ126-178, LQ126-180, LQ126-181, LQ126-182, LQ126-183, LQ126-184, LQ126-185, LQ126-186, LQ141-1, LQ141-2, LQ141-3, LQ141-4, LQ141-5, LQ141-6, LQ141-7, LQ141-8, LQ141-9, LQ141-10, LQ141-11, LQ141-12, LQ141-13, LQ141-14, LQ141-15, LQ141-16, LQ141-17, LQ141-18, LQ141-19, LQ141-20, LQ141-21, LQ141-22, LQ141-24, LQ141-26, LQ141-27, LQ141-28, LQ141-29, LQ141-33, LQ141-36, LQ141-37, LQ141-38, LQ141-39, LQ141-42, LQ141-43, LQ141-44, LQ141-45, LQ141-46, LQ141-47, LQ141-48, LQ141-49, LQ141-52 and LQ141-57.

In some aspects of the disclosed methods, the bivalent compounds can be administered by any of several routes of administration including, e.g., orally, parenterally, intradermally, subcutaneously, topically, and/or rectally.

Any of the above-described methods can further include treating the subject with one or more additional therapeutic regimens for treating cancer. The one or more additional therapeutic regimens for treating cancer can be, e.g., one or more of surgery, chemotherapy, radiation therapy, hormone therapy, or immunotherapy.

This disclosure additionally provides a method for identifying a bivalent compound which mediates degradation/disruption of ENL, the method including providing a heterobifunctional test compound including a ENL ligand conjugated to a degradation/disruption tag, contacting the heterobifunctional test compound with a cell (e.g., a cancer cell such as a ENL-mediated cancer cell) including a ubiquitin ligase and ENL.

As used herein, the terms “about” and “approximately” are defined as being within plus or minus 10% of a given value or state, preferably within plus or minus 5% of said value or state. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1. ENL and its YEATS domain are essential for the maintenance and progression of leukemia in vitro and in vivo. FIG. 1A, Depletion of ENL, but not AF9, suppresses the cell growth of MOLM13 and MV4; 11, two MLL-rearranged leukemia cell lines. FIG. 1B, Depletion of ENL in MOLM13 cells delays leukemia progression in xenograft recipient mice. FIG. 1C, The function of ENL in xenografted tumor progression depends on its YEATS domain.

FIG. 2. Precursors of ENL degraders show strong inhibition to the ENL YEATS domain binding to acetylated histone peptide in AlphaScreen assay. FIG. 2A, Inhibitory effect of precursors tested at 1 μM. FIG. 2B, IC₅₀ of selected ENL degrader precursors measured in AlphaScreen assay.

FIG. 3A-E. Effect of ENL degraders on ENL-dependent MV4; 11 cell growth after 72 h treatment at 0.4, 2, 10 and 50 μM.

FIG. 4. Dose-dependent cell growth inhibition by selected ENL degraders and SGC-iMLLT in ENL-dependent MV4; 11 cells and ENL-independent Jurkat cells after 72 h treatment at 0.4, 2, 10 and 50 μM.

FIG. 5. ENL protein degradation induced by the same panel of ENL degraders as shown in FIG. 4 in MV4; 11 cells treated with 1 μM and 10 μM compounds for 24 h.

FIG. 6. Western blots showing that ENL degraders, LQ076-122, LQ081-108 and LQ081-109, concentration-dependently reduce ENL levels at 0, 0.25, 0.5, 1, 1.5, 2, 2.5, 3, 4 and 8 μM doses in MV4; 11 cells after 24 h treatment.

FIG. 7. Western blots showing that ENL degraders, LQ076-122 and LQ081-108, concentration-dependently reduce ENL levels at 0, 0.25, 0.5, 1, 1.5, 2, 2.5, 3, 4 and 8 μM doses in MOLM13 cells after 24 h treatment.

FIG. 8. Western blots showing that ENL degraders LQ076-122 and LQ081-108, but not their corresponding negative control compounds (LQ081-107 and LQ081-106) or SGC-iMLLT, concentration-dependently reduce ENL levels at 0.3, 1, 3 and 10 μM doses in MV4; 11 cells after 12 and 24 h treatment.

FIG. 9. Western blots showing that LQ076-122 time-dependently reduces ENL levels in MV4; 11 cells at 4 μM dose.

FIG. 10. Western blots showing that LQ076-122 time-dependently reduces ENL levels in MOLM13 cells at 8 μM dose.

FIG. 11. Western blots showing that LQ076-122 selectively reduces the ENL protein level, but not the protein level of another YEATS domain-containing protein GAS41, in MV4; 11 cells.

FIG. 12A-B. Effect of selected ENL degraders on ENL-dependent MV4; 11 cell growth after 72 h treatment at 0.5, 1, 2 and 4 μM.

FIG. 13A-C. ENL degraders LQ076-122, LQ081-108 and LQ081-109, but not the negative control compounds (LQ108-4, LQ081-106, LQ108-141, LQ081-158 and LQ108-142) or SGC-iMLLT, suppress cell growth specifically of the ENL-dependent MV4; 11 (FIG. 13A) and MOLM13 (FIG. 13B) leukemia cells, but not the ENL-independent Jurkat cells (FIG. 13C) after 72 h treatment at 0.5, 1, 2 and 4 μM.

FIG. 14A-B. ENL degraders LQ076-122 (FIG. 14A) and LQ081-108 (FIG. 14B) concentration-dependently suppress ENL target gene expression in MOLM13 cells.

FIG. 15. ENL degrader LQ076-122 suppresses ENL target gene expression in a concentration- and time-dependent manner in MV4; 11 cells.

FIG. 16A-B. ENL degrader LQ076-122, but not the negative control compound LQ108-4 or SGC-iMLLT, induces apoptosis in MV4; 11 (FIG. 16A) and MOLM13 (FIG. 16B) cells after 24 h treatment at 1, 2, and 4 μM.

FIG. 17. Plasma concentration of ENL degrader LQ076-122 over 12 h following a single 50 mg/kg IP injection in mice.

FIG. 18. ENL degrader LQ076-122 significantly delays the leukemia progression in an MV4; 11 disseminated xenograft model. FIG. 18A, Bioluminescence imaging of intravenously xenografted MV4; 11-Luc cells at different time points upon LQ076-122 or vehicle treatment.

FIG. 18B, Quantification of the mean radiance of bioluminescence signal.

FIG. 19A-D. ENL protein degradation induced by ENL degraders in MV4; 11 cells stably expressing 3Flag-HA-tagged ENL. Cells were treated with 1 μM and 10 μM compounds for 24 h, DMSO was used as negative control. Degradation of ectopic 3Flag-HA-ENL was detected by Western blot using anti-HA tag antibody.

FIG. 20A-B. ENL protein degradation induced by selected ENL degraders in MV4; 11 cells stably expressing 3Flag-HA-tagged ENL. Cells were treated with 1 μM and 10 μM compounds for 6 h, DMSO was used as negative control. Degradation of ectopic 3Flag-HA-ENL was detected by Western blot using anti-HA tag antibody.

FIG. 21. ENL protein degradation induced by selected ENL degraders in MV4; 11 cells. Cells were treated with 1 μM and 10 μM compounds for 6 h, DMSO was used as negative control. Degradation of endogenous ENL was detected by Western blot using anti-ENL antibody.

FIG. 22. Western blots showing that ENL degraders, LQ108-69, LQ108-71, LQ108-72, LQ126-62 and LQ126-63, concentration-dependently reduce ENL levels at 0, 1 nM, 10 nM, 100 nM, 1 μM, and 10 μM doses in MV4; 11, MOLM13 and Jurkat cells after 6 h treatment.

FIG. 23. Western blots showing that ENL degraders, LQ108-69, LQ108-70, LQ108-71, LQ108-72, LQ126-62 and LQ126-63, reduce ENL levels at 1 μM dose in MV4; 11, MOLM13 and Jurkat cells after 48 and 72 h treatment.

FIG. 24. MG132 treatment partially blocks the ENL degradation induced by degraders LQ108-63, LQ108-69, LQ108-70, LQ126-62 and LQ126-63 in MV4; 11 cells. Cells were treated with 1 μM of ENL degrader with or without 1 μM MG132 for 6 h.

FIG. 25. Effect of ENL degraders on ENL-dependent MV4; 11 cell growth after 72 h treatment at 0, 1.25, 2.5, 5 and 10 μM doses.

FIG. 26. Effect of ENL degrader LQ126-63 on the growth of ENL-dependent MV4; 11 cells and ENL-independent Jurkat cells after 3 days (A) and 6 days (B) of treatment at 0, 10 nM, 100 nM, 1 μM and 10 μM doses.

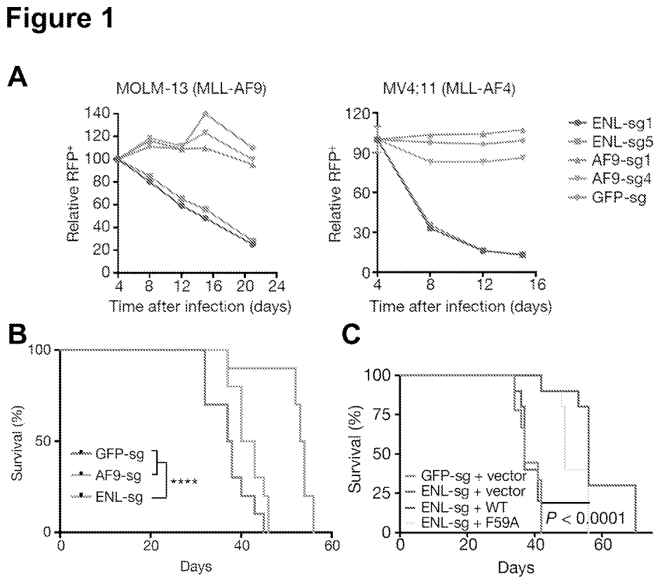

### DETAILED DESCRIPTION

The present disclosure is based, in part, on the discovery that novel heterobifunctional molecules which degrade ENL, ENL fusion proteins, and/or ENL mutant proteins are useful in the treatment of ENL-mediated diseases including but not limited to acute leukemia, mixed lineage leukemia (MLL)-rearranged leukemias and Wilms' tumor.

Successful strategies for selective degradation/disruption of the target protein induced by a bifunctional molecule include recruiting an E3 ubiquitin ligase and mimicking protein misfolding with a hydrophobic tag (Buckley and Crews, 2014). PROTACs (PROteolysis TArgeting Chimeras) are bivalent molecules with one moiety that binds an E3 ubiquitin ligase and another moiety that binds the protein target of interest (Buckley and Crews, 2014). The induced proximity leads to selective ubiquitination of the target followed by its degradation at the proteasome. Several types of high affinity small-molecule E3 ligase ligands have been identified/developed: They include (1) immunomodulatory drugs (IMiDs) such as thalidomide and pomalidomide, which bind cereblon (CRBN or CRL4^{CRBN}), a component of a cullin-RING ubiquitin ligase (CRL) complex (Bondeson et al., 2015; Chamberlain et al., 2014; Fischer et al., 2014; Ito et al., 2010; Winter et al., 2015); (2) VHL-1, a hydroxyproline-containing ligand, which binds van Hippel-Lindau protein (VHL or CRL2^{VHL}), a component of another CRL complex (Bondeson et al., 2015; Buckley et al., 2012a; Buckley et al., 2012b; Galdeano et al., 2014; Zengerle et al., 2015); (3) compound 7, which selectively binds KEAP1, a component of a CRL3 complex (Davies et al., 2016); (4) AMG232, which selectively binds MDM2, a heterodimeric RING E3 ligase (Sun et al., 2014); and (5) LCL161, which selectively binds IAP, a homodimeric RING E3 ligase (Ohoka et al., 2017; Okuhira et al, 2011; Shibata et al., 2017). The degrader technology has been successfully applied to degradation of multiple targets (Bondeson et al., 2015; Buckley et al., 2015; Lai et al., 2016; Lu et al., 2015; Winter et al., 2015; Zengerle et al., 2015), but not to degradation of ENL. In addition, a hydrophobic tagging approach, which utilizes a bulky and hydrophobic adamantyl group, has been developed to mimic protein misfolding, leading to the degradation of the target protein by proteasome (Buckley and Crews, 2014). This approach has also been successfully applied to selective degradation of the pseudokinase Her3 (Xie et al., 2014), but not to degradation of ENL proteins.

As discussed in the following examples, this disclosure provides specific examples of novel ENL degraders/disruptors, and examined the effect of exemplary degraders/disruptors on reducing ENL protein levels, and inhibiting MLL-rearranged leukemia cells proliferation. The results indicated that these novel compounds can be beneficial in treating human disease, especially acute leukemia, MLL-rearranged leukemia.

Current compounds targeting ENL generally focus on blocks the interaction between the ENL YEATS domain and acetylated histone H3, and have no effect in inhibiting the growth of ENL-dependent MLL-rearranged leukemia cells. In the present disclosure a different approach was taken: to develop compounds that they effectively degrade ENL in cells and reduce the proliferation of ENL-dependent MLL-rearranged leukemia cells in vitro and in vivo. Strategies for inducing protein degradation include recruiting E3 ubiquitin ligases, mimicking protein misfolding with hydrophobic tags, and inhibiting chaperones. For example, a thalidomide-JQ1 bivalent compound has been used to hijack the cereblon E3 ligase, inducing highly selective BET protein degradation in vitro and in vivo and resulting in a demonstrated delay in leukemia progression in mice (Winter et al., 2015). Similarly, BET protein degradation has also been induced via another E3 ligase, VHL (Zengerle et al., 2015). Partial degradation of the Her3 protein has been induced using an adamantane-modified compound (Xie et al., 2014). Such an approach, based on the use of bivalent molecules, permits more flexible regulation of protein levels in vitro and in vivo compared with techniques such as gene knockout or knockdown via RNA interference. Unlike gene knockout or knockdown, this chemical approach provides an opportunity to study dose and time dependency in a disease model by varying the concentrations and frequencies of administration of the relevant compound.

This disclosure includes all stereoisomers, geometric isomers, tautomers and isotopes of the structures depicted and compounds named herein. This disclosure also includes compounds described herein, regardless of how they are prepared, e.g., synthetically, through biological process (e.g., metabolism or enzyme conversion), or a combination thereof.

This disclosure includes pharmaceutically acceptable salts of the structures depicted and compounds named herein.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms. In some embodiments, the compound includes at least one fluorine atom. In some embodiments, the compound includes two or more fluorine atoms. In some embodiments, the compound includes 1-2, 1-3, 1-4, 1-5, or 1-6 fluorine atoms. In some embodiments, all of the hydrogen atoms in a compound can be replaced or substituted by fluorine atoms.

### Degraders

In some aspects, the present disclosure provides bivalent compounds, also referred to herein as degraders, comprising an ENL ligand (or targeting moiety) conjugated to a degradation tag. Linkage of the ENL ligand to the degradation tag can be direct, or indirect via a linker.

As used herein, the terms “Eleven-Nineteen Leukemia (ENL) ligand” or “ENL ligand” or “ENL targeting moiety” are to be construed broadly, and encompass a wide variety of molecules ranging from small molecules to large proteins that associate with or bind to ENL. The ENL ligand or targeting moiety can be, for example, a small molecule compound (i.e., a molecule of molecular weight less than about 1.5 kilodaltons (kDa)), a peptide or polypeptide, nucleic acid or oligonucleotide, carbohydrate such as oligosaccharides, or an antibody or fragment thereof.

The ENL ligand or targeting moiety can be derived from an ENL inhibitor (e.g., SGC-iMLLT), which can block the interaction between the ENL YEATS domain and acetylated histone H3 in vitro and in cells. As used herein, an “inhibitor” refers to an agent that restrains, retards, or otherwise causes inhibition of a physiological, chemical or enzymatic action or function. As used herein an inhibitor causes a decrease in enzyme activity of at least 5%. An inhibitor can also or alternatively refer to a drug, compound, or agent that prevents or reduces the expression, transcription, or translation of a gene or protein. An inhibitor can reduce or prevent the function of a protein, e.g., by binding to or activating/inactivating another protein or receptor.

Exemplary ENL ligands include, but are not limited to, the compounds listed below:

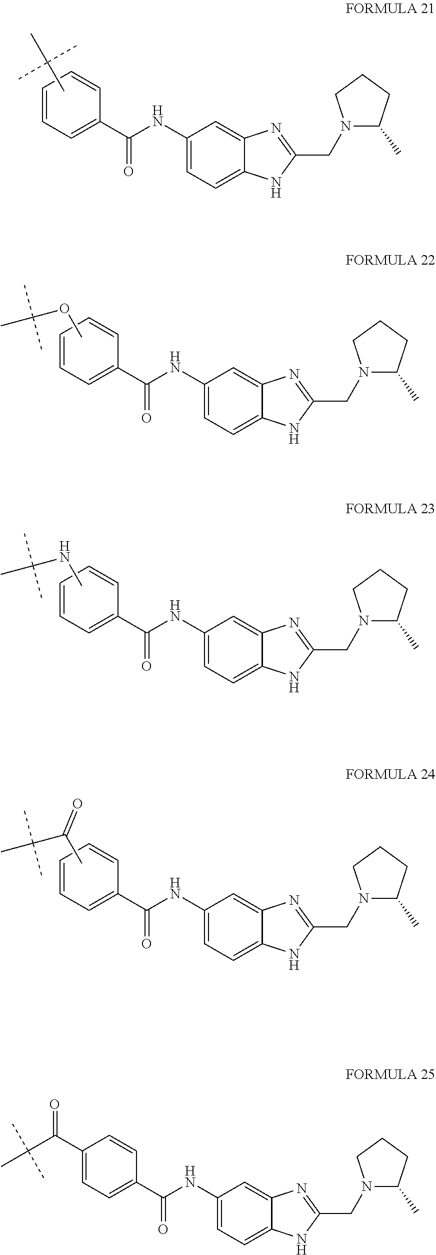

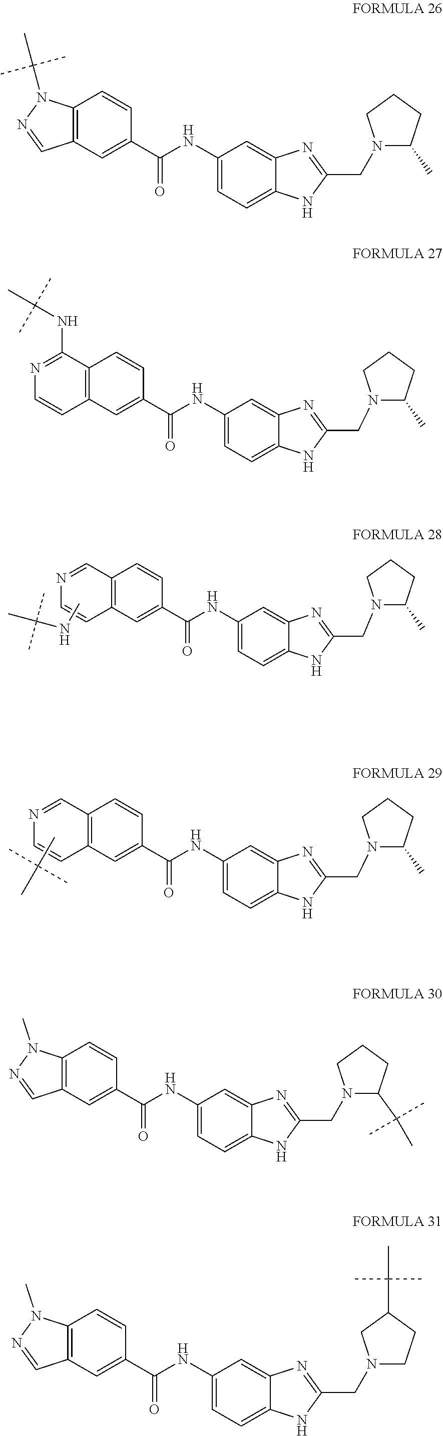

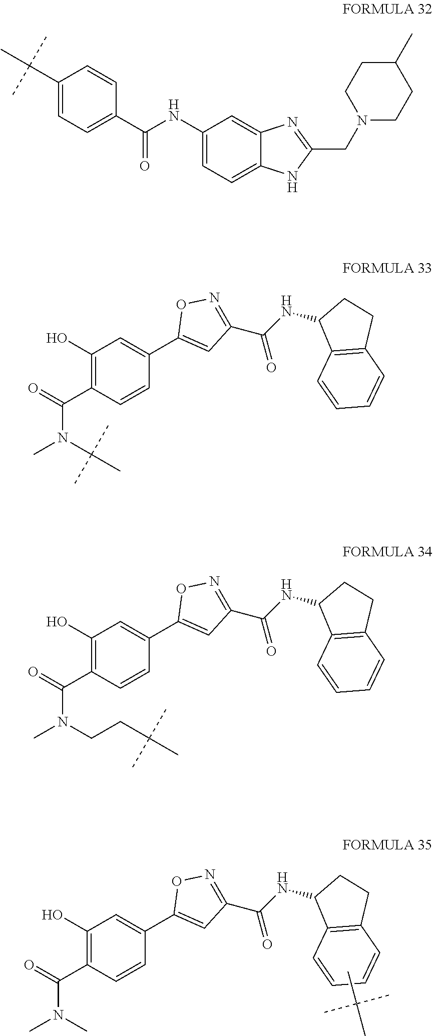

As used herein, the term “degradation/disruption tag” refers to a compound, which associates with or binds to a ubiquitin ligase for recruitment of the corresponding ubiquitination machinery to ENL or induces ENL protein misfolding and subsequent degradation at the proteasome or loss of function.

In some aspects, the degradation/disruption tags of the present disclosure include, e.g., thalidomide, pomalidomide, lenalidomide, VHL-1, adamantane, 1-((4,4,5,5,5-pentafluoropentyl)sulfinyl)nonane, nutlin-3a, RG7112, RG7338, AMG232, AA-115, bestatin, MV-1, LCL161, FK506, rapamycin and/or analogs thereof.

As used herein, a “linker” is a bond, molecule, or group of molecules that binds two separate entities to one another. Linkers can provide for optimal spacing of the two entities. The term “linker” in some aspects refers to any agent or molecule that bridges the ENL ligand to the degradation/disruption tag. One of ordinary skill in the art recognizes that sites on the ENL ligand or the degradation/disruption tag, which are not necessary for the function of the degraders of the present disclosure, are ideal sites for attaching a linker, provided that the linker, once attached to the conjugate of the present disclosure, does not interfere with the function of the degrader, i.e., its ability to target ENL and its ability to recruit a ubiquitin ligase.

The length of the linker of the bivalent compound can be adjusted to minimize the molecular weight of the disruptors/degraders and avoid any potential clash of the ENL ligand or targeting moiety with either the ubiquitin ligase or the induction of ENL misfolding by the hydrophobic tag at the same time.

In some aspects, the degradation/disruption tags of the present disclosure include, for example, thalidomide, pomalidomide, lenalidomide, VHL-1, adamantane, 1-((4,4,5,5,5-pentafluoropentyl)sulfinyl)nonane, nutlin-3a, RG7112, RG7338, AMG 232, AA-115, bestatin, MV-1, LCL161, FK506, rapamycin and analogs thereof. The degradation/disruption tags can be attached to any portion of the structure of an ENL ligand or targeting moiety (SGC-iMLLT) with linkers of different types and lengths in order to generate effective bivalent compounds. In particular, attaching VHL1, pomalidomide, to any portion of the molecule can recruit the E3 ligase to ENL.

The bivalent compounds disclosed herein can selectively reduce the proliferation of ENL-mediated disease cells in vitro and in vivo.

Additional bivalent compounds (i.e., ENL degraders/disruptors) can be developed using the principles and methods disclosed herein. For example, other linkers, degradation tags, and ENL binding/inhibiting moieties can be synthesized and tested. Non-limiting examples of ENL disruptors/degraders (e.g., bivalent compounds) are shown in Table 1 (below). The left portion of each ENL disruptors/degrader compound as shown binds to ENL (as SGC-iMLLT do), and the right portion of each compound recruits for the ubiquitination machinery to ENL, which induces the poly-ubiquitination and degradation of ENL at the proteasome.

More specifically, the present disclosure provides a bivalent compound including an ENL ligand conjugated to a degradation/disruption tag.

In some aspects, the ENL degraders/disruptors have the form “PI-linker-EL”, as shown below:

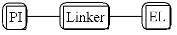

wherein PI (protein of interest) comprises an ENL ligand and EL (E3 ligase) comprises a degradation/disruption tag (e.g., E3 ligase ligand). Exemplary ENL ligands (PI), exemplary degradation/disruption tags (EL), and exemplary linkers (Linker) are illustrated below:

ENL Ligands

In an embodiment, ENL ligands include a moiety according to FORMULA 1:

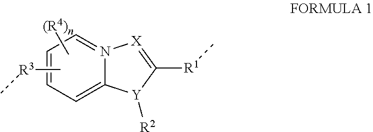

wherein
the “Linker” moiety of the bivalent compound is attached independently to R¹ or R³
X and Y are independently selected from C, O or N;

R¹ is selected from H, halogen, OR⁵, SR⁵, C₁-C₈ alkylene NR⁵R⁶, CH₂CH₂NR⁵R⁶, NR⁵R⁶, C(O)R⁵, C(O)OR⁵, C(S)OR⁵, C(O)NR⁵R⁶, S(O)R⁵, S(O)₂R⁵, S(O)₂NR⁵R⁶, NR⁷C(O)OR⁶, NR⁷C(O)R⁶, NR⁷S(O)R⁶, NR⁷S(O)₂R⁶, or unsubstituted or optionally substituted C₁-C₈ alkyl, C₁-C₈ haloalkyl, C₁-C₈ hydroxyalkyl, C₃-C₁₀ cycloalkyl, C₃-C₁₀ heterocyclyl, optionally substituted C₁-C₈alkoxyC₁-C₈alkyl, optionally substituted C₁-C₈alkylaminoC₁-C₈alkyl.

R² is independently selected from hydrogen, halogen, oxo, CN, NO₂, OR⁸, SR⁸, NR⁸R⁹, C(O)R⁸, C(O)OR⁸, C(S)OR⁸, C(O)NR⁸R⁹, S(O)R⁸, S(O)₂R⁸, S(O)₂NR⁸R⁹, NR¹⁰C(O)OR⁹, NR¹⁰C(O)R⁹, NR¹⁰S(O)R⁹, NR¹⁰S(O)₂R⁹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₃-C₈ heterocyclyl, optionally substituted aryl, and optionally substituted heteroaryl;

R³ is unsubstituted or optionally substituted with one or more groups selected from hydrogen, halogen, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR¹¹R¹², C(O)R¹¹, C(O)OR¹¹, C(O)NR¹¹R¹², S(O)R¹¹, S(O)₂R¹¹, S(O)₂NR¹¹R¹², NR¹³C(O)OR¹², NR¹³C(O)R¹², NR¹³S(O)R¹², NR¹³S(O)₂R¹², optionally substituted C₆-C₁₀ aryl and optionally substituted C₅-C₁₀ heteroaryl.

each R⁴ is independently selected from null, hydrogen, halogen, oxo, CN, NO₂, OR¹⁴, SR¹⁴, NR¹⁴R¹⁵, OCOR¹⁴, OCO₂R¹⁴, OCONR¹⁴R¹⁵, COR¹⁴, CO₂R¹⁵, CONR¹⁴R¹⁵, SOR¹⁴, SO₂R¹⁴, SO₂NR¹⁴R¹⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;

R⁵, R⁶, R⁷, R⁸, R⁹, R¹⁰ R¹¹, R¹², R¹³ R¹⁴, R¹⁵ are independently selected from H, C₁-C₈ alkyl, C₁-C₈ haloalkyl, C₁-C₈ hydroxyalkyl, C₃-C₁₀ cycloalkyl, C₃-C₁₀ heterocyclyl, C(O) C₁-C₈ alkyl, C(O) C₁-C₈ haloalkyl, C(O) C₁-C₈ hydroxyalkyl, C(O) C₃-C₁₀ cycloalkyl, C(O) C₃-C₁₀ heterocyclyl, optionally substituted C₆-C₁₀ aryl or C₅-C₁₀ heteroaryl.

R⁵ and R⁶, R⁶ and R⁷, R⁸ and R⁹, R⁸ and R¹⁰, R⁹ and R¹⁰, R¹¹ and R¹², R¹¹ and R¹³, R¹² and R¹³, R¹⁴ and R¹⁵, together with the nitrogen atom to which they connected can independently form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring.
n is independently selected from 0, 1, 2, 3, 4 and 5;
and pharmaceutically acceptable salts thereof.

In an embodiment, ENL ligands include a moiety according to FORMULA 1A

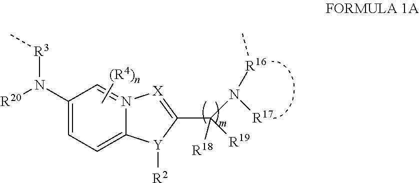

wherein
the “Linker” moiety of the bivalent compound is attached independently to R³ or R¹⁶
X and Y are independently selected from C, O or N;
the definitions of R², R³, R⁴ are the same as for FORMULA 1;

R¹⁶, R¹⁷ is selected from hydrogen, C₁-C₈ alkyl, C₁-C₈ haloalkyl, C₁-C₈ hydroxyalkyl, C₃-C₁₀ cycloalkyl, C₃-C₁₀ heterocycloalkyl, C₆-C₁₀ aryl, C₅-C₁₀ heteroaryl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, C(O)C₆-C₁₀ aryl, C(O)C₅-C₁₀ heteroaryl

or

R¹⁶ and R¹⁷ together with the nitrogen atom to which they connected can independently form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring.

R¹⁸, R¹⁹ are independently selected from hydrogen, halogen, CN, OH, NH₂, optionally substituted C₁-C₈ alkyl, optionally substituted C₃-C₈ cycloalkyl, optionally substituted C₃-C₈ cycloalkoxy, optionally substituted C₁-C₈alkoxyC₁-C₈alkyl, optionally substituted C₁-C₈alkylaminoC₁-C₈alkyl, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;

R²⁰ is selected from hydrogen, optionally substituted C₁-C₈ alkyl, optionally substituted C₃-C₈ cycloalkyl, optionally substituted C₃-C₈ cycloalkoxy, optionally substituted C₃-C₈ heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl.
m, n, are independently selected from 0, 1, 2, 3, and 4;

In an embodiment, ENL ligands include a moiety according to FORMULA 1B, 1C, 1D, 1E

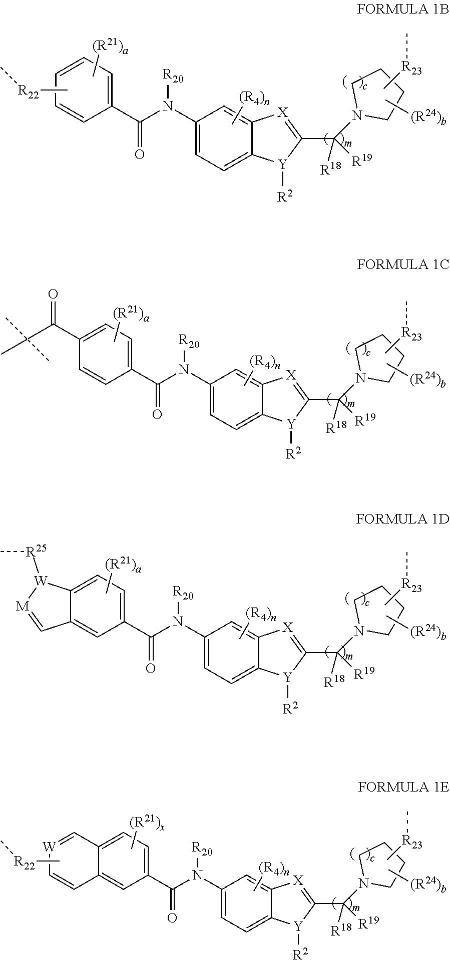

wherein
the “Linker” moiety of the bivalent compound is attached independently to R²², R²³, R²⁵.
X and Y are independently selected from C, O or N;
M and W are independently selected from C or N.
the definitions of R², R⁴, R¹⁸, R¹⁹, R²⁰ are the same as for FORMULA 1A;
   each R²¹ is independently selected from null, hydrogen, halogen, oxo, CN, NO₂, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;

R²² is unsubstituted or optionally substituted with one or more groups selected from halo, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR²⁶R²⁷, C₁-C₈NR²⁶R²⁷, C(O)R²⁶, C(O)OR²⁶, C(O)NR²⁶R²⁷, S(O)R²⁶, S(O)₂R²⁶, S(O)₂NR²⁶R²⁷, NR²⁶C(O)OR²⁷, NR²⁸C(O)R²⁷, NR²⁸S(O)R²⁷, NR²⁸S(O)₂R²⁷.

R²³ is unsubstituted or optionally substituted with one or more groups selected from halo, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR²⁹R³⁰, C(O)R²⁹, C(O)OR²⁹, C(O)NR²⁹R³⁰, S(O)R²⁹, S(O)₂R²⁹, S(O)₂NR²⁹R³⁰, NR³¹C(O)OR²⁹, NR³¹C(O)R²⁹, NR³¹S(O)R²⁹, NR³¹S(O)₂R²⁹.
each R²⁴ is independently selected from null, hydrogen, halogen, oxo, CN, NO₂, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;

R²⁵ is unsubstituted or optionally substituted with one or more groups selected from halo, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR³²R³³, C(O)R³², C(O)OR³², C(O)NR³²R³³, S(O)R³², S(O)₂R³², S(O)₂NR³²R³³, NR³⁴C(O)OR³², NR³⁴C(O)R³², NR³⁴S(O)R³², NR³⁴S(O)₂R³².

R²⁶, R²⁷, R²⁸, R²⁹, R³⁰, R³¹ R³², R³³, R³⁴ are independently selected from H, C₁-C₈ alkyl, C₁-C₈ haloalkyl, C₁-C₈ hydroxyalkyl, C₃-C₁₀ cycloalkyl, C₃-C₁₀ heterocyclyl, C(O) C₁-C₈ alkyl, C(O) C₁-C₈ haloalkyl, C(O) C₁-C₈ hydroxyalkyl, C(O) C₃-C₁₀ cycloalkyl, C(O) C₃-C₁₀ heterocyclyl, optionally substituted C₆-C₁₀ aryl or C₅-C₁₀ heteroaryl.

R²⁶ and R²⁷, R²⁷ and R²⁸, R²⁹ and R³⁰, R²⁹ and R³¹, R³² and R³³, R³² and R³⁴, together with the nitrogen atom to which they connected can independently form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring.
m, n, a, b are independently selected from 0, 1, 2, 3, and 4;
c is independently selected from 0, 1, 2, 3, 4, 5 and 6.

In an embodiment, ENL ligands include a moiety according to FORMULA 1F:

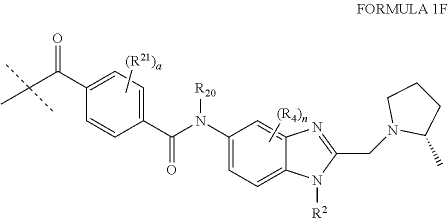

wherein
the “Linker” moiety of the bivalent compound is attached to the carbonyl group indicated with dotted line
the definitions of R², R⁴, R²⁰, R²¹ are the same as for FORMULA 1B;
n, a are independently selected from 0, 1, 2, 3, and 4;

In an embodiment, ENL ligands include a moiety according to FORMULA 2.

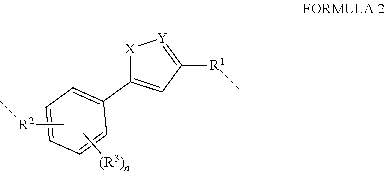

wherein
the “Linker” moiety of the bivalent compound is attached independently to R¹ or R²
X and Y are independently selected from C, O or N;

R¹ is selected from hydrogen, halogen, OR⁴, SR⁴, C₁-C₈ alkylene NR⁴R⁵, C(O)R⁴, C(O)OR⁴, C(S)OR⁴, C(O)NR⁴R⁵, S(O)R⁴, S(O)₂R⁴, S(O)₂NR⁴R⁵, NR⁶C(O)OR⁴, NR⁶C(O)R⁴, NR⁶S(O)R⁴, NR⁶S(O)₂R⁴, or unsubstituted or optionally substituted C₁-C₈ alkyl, C₁-C₈ haloalkyl, C₁-C₈ hydroxyalkyl, C₃-C₁₀ cycloalkyl, C₃-C₁₀ heterocyclyl, or fused C₃-C₁₀ cycloalkyl, C₃-C₁₀ heterocyclyl.

R² is selected from hydrogen, halogen, CN, NO₂, or unsubstituted or optionally substituted C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR⁷R⁸, C(O)R⁷, C(O)OR⁷, C(O)NR⁷R⁸, S(O)R⁷, S(O)₂R⁷, S(O)₂NR⁷R⁸, NR⁹C(O)OR⁷, NR⁹C(O)R⁷, NR⁹S(O)R⁷, NR⁹S(O)₂R⁷, optionally substituted C₆-C₁₀ aryl and optionally substituted C₅-C₁₀ heteroaryl.
each R³ is independently selected from null, hydrogen, halogen, oxo, OH, CN, NO₂, OR¹⁰, SR¹⁰, NR¹⁰R¹¹, OCOR¹⁰, OCO₂R¹⁰, OCONR¹⁰R¹¹, COR¹, CO₂R¹⁰, CONR¹⁰R¹¹, SOR¹⁰, SO₂R¹⁰, SO₂NR¹⁰R¹¹, NR¹²C(O)OR¹⁰, NR¹²C(O)R¹⁰, NR¹²S(O)R¹⁰, NR¹²S(O)₂R¹⁰, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;
wherein

R⁴, R⁵, R⁶, R⁷, R⁸, R⁹, R¹⁰ R¹¹, R¹² are independently selected from H, C₁-C₈ alkyl, C₁-C₈ haloalkyl, C₁-C₈ hydroxyalkyl, C₃-C₁₀ cycloalkyl, C₃-C₁₀ heterocyclyl, C(O) C₁-C₈ alkyl, C(O) C₁-C₈ haloalkyl, C(O) C₁-C₈ hydroxyalkyl, C(O) C₃-C₁₀ cycloalkyl, C(O) C₃-C₁₀ heterocyclyl, optionally substituted C₆-C₁₀ aryl or C₅-C₁₀ heteroaryl.

R⁴ and R⁵, R⁴ and R⁶, R⁷ and R⁸, R⁷ and R⁹, R¹⁰ and R¹¹, R¹⁰ and R¹², together with the nitrogen atom to which they connected can independently form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring.
n is independently selected from 0, 1, 2, 3, 4;

In an embodiment, ENL ligands include a moiety according to FORMULA 2A and 2B.

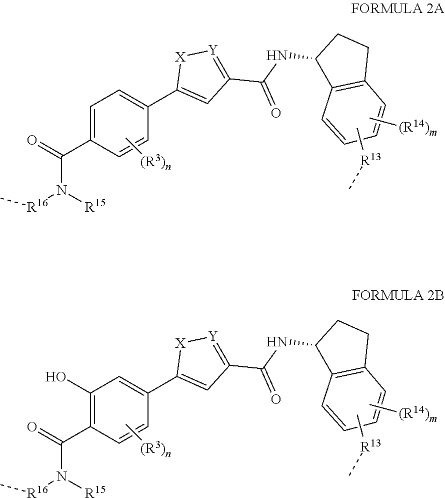

wherein
the “Linker” moiety of the bivalent compound is attached independently to R¹³ or R¹⁶
X and Y are independently selected from C, O or N;
the definitions of R³ is the same as for FORMULA 2;

R¹³ is selected from hydrogen, halogen OR¹⁷, SR¹⁷, C₁-C₈ alkylene NR¹⁷R¹⁸, NR¹⁷R¹⁸, C(O)R¹⁷, C(O)OR¹⁷, C(S)OR¹⁷, C(O)NR¹⁷R¹⁸, S(O)R¹⁷, S(O)₂R¹⁷, S(O)₂NR¹⁷R¹⁵, NR¹⁹C(O)OR¹⁷, NR¹⁹C(O)R¹⁷, NR¹⁹S(O)R¹⁷, NR¹⁹S(O)₂R¹⁷, or unsubstituted or optionally substituted C₁-C₈ alkyl, C₁-C₈ haloalkyl, C₁-C₈ hydroxyalkyl, C₃-C₁₀ cycloalkyl, C₃-C₁₀ heterocyclyl.

each R¹⁴ is independently selected from unsubstituted or optionally substituted with one or more groups selected from hydrogen, halogen, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR²⁰R²¹, C(O)R²⁰, C(O)OR²⁰, C(O)NR²⁰R²¹, S(O)R²⁰, S(O)₂R²⁰, S(O)₂NR²⁰R²¹, NR²²C(O)OR²⁰, NR²²C(O)R²⁰, NR²²S(O)R²⁰, NR²²S(O)₂R²⁰, optionally substituted C₆-C₁₀ aryl and optionally substituted C₅-C₁₀ heteroaryl.

R¹⁵ is selected from hydrogen, optionally substituted C₁-C₈ alkyl, optionally substituted C₃-C₈ cycloalkyl, optionally substituted C₃-C₈ cycloalkoxy, optionally substituted C₃-C₈ heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl.

R¹⁶ is selecy from null, hydrogen, halogen, oxo, CN, NO₂, OR²³, SR²³, NR²³R²⁴, OCOR²³, OCO₂R²³, OCONR²³R²⁴, COR²³, CO₂R²³, CONR²³R²⁴, SOR²³, SO₂R²³, SO₂NR²³R²⁴, NR²⁵C(O)OR²³, NR²⁵C(O)R²³, NR²⁵S(O)R²³, NR²⁵S(O)₂R²³, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;

wherein

R¹⁷, R¹⁸, R¹⁹, R²⁰, R²¹, R²², R²³, R²⁴, R²⁵ are independently selected from H, C₁-C₈ alkyl, C₁-C₈ haloalkyl, C₁-C₈ hydroxyalkyl, C₃-C₁₀ cycloalkyl, C₃-C₁₀ heterocyclyl, C(O) C₁-C₈ alkyl, C(O) C₁-C₈ haloalkyl, C(O) C₁-C₈ hydroxyalkyl, C(O) C₃-C₁₀ cycloalkyl, C(O) C₃-C₁₀ heterocyclyl, optionally substituted C₆-C₁₀ aryl or C₅-C₁₀ heteroaryl.

R¹⁷ and Rig, R¹⁷ and R¹⁹, R²⁰ and R²¹, R²⁰ and R²², R²³ and R²⁴, R²³ and R²⁵, together with the nitrogen atom to which they connected can independently form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring.
m, n is independently selected from 0, 1, 2, 3, 4;

In an embodiment, ENL ligands include a moiety according to FORMULA 2C.

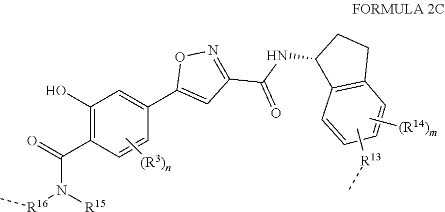

Wherein
the “Linker” moiety of the bivalent compound is attached independently to R¹³ or R¹⁶
the definitions of R³, R¹³, R¹⁴, R¹⁵ an R¹⁶ is the same as for FORMULA 2A and 2C;

In an embodiment, ENL ligands include a moiety according to FORMULA 3.

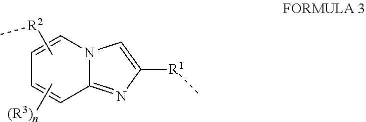

Wherein
the “Linker” moiety of the bivalent compound is attached independently to R¹ or R²
the definitions of R¹, R² and R³ are the same as for FORMULA 2;

In an embodiment, ENL ligands include a moiety according to FORMULA 3A.

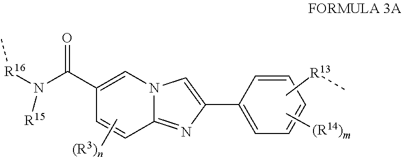

wherein
the “Linker” moiety of the bivalent compound is attached independently to R¹³ or R¹⁶
the definitions of R³, R¹³, R¹⁴, R¹⁵ and R¹⁶ are the same as for FORMULA 2A;
n is selected from 0, 1, 2, 3; and
m is selected from 0, 1, 2, 3, 4; and
and pharmaceutically acceptable salts thereof.

In an embodiment, (ENL) ligands are selected from the group consisting of:

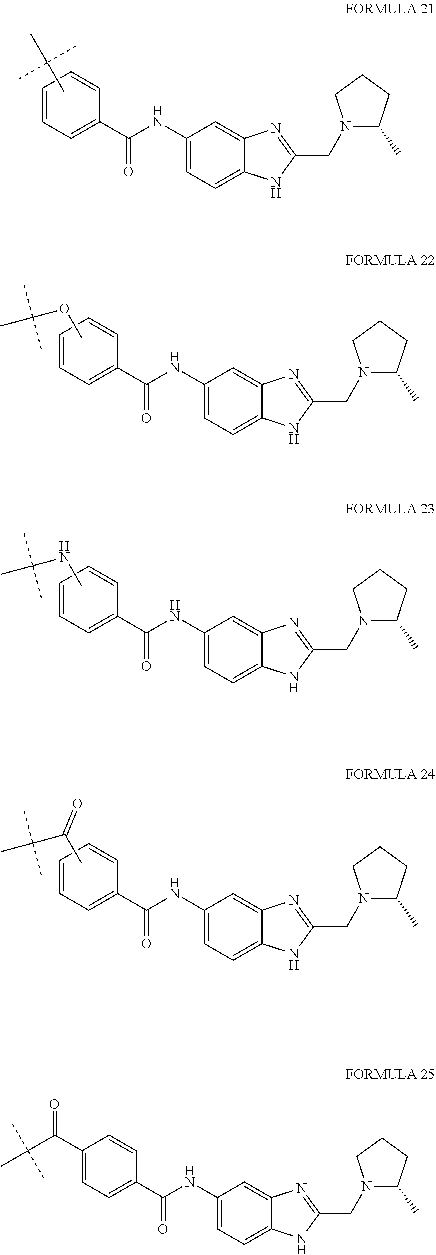

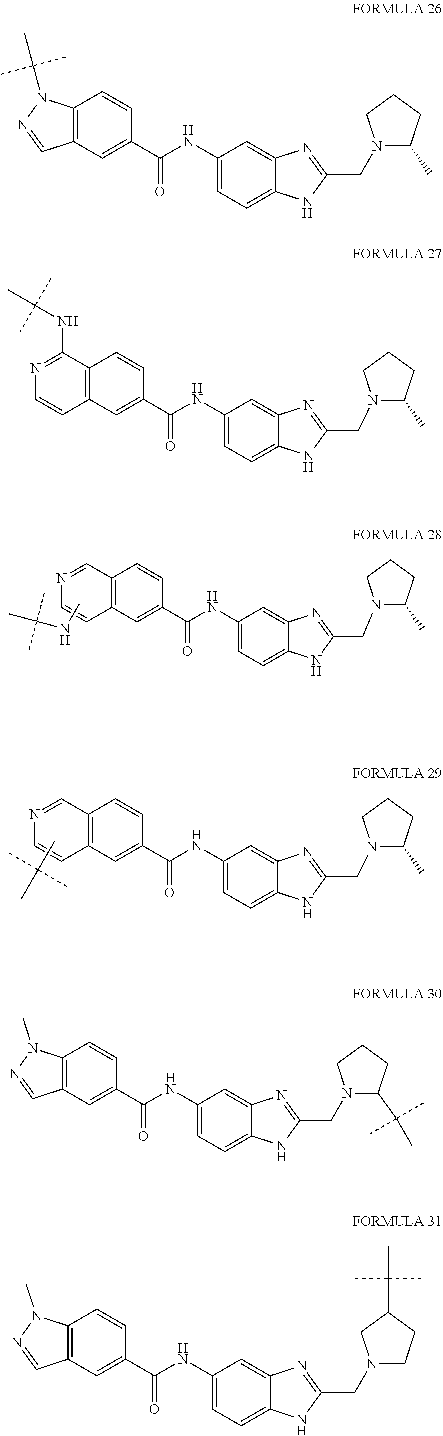

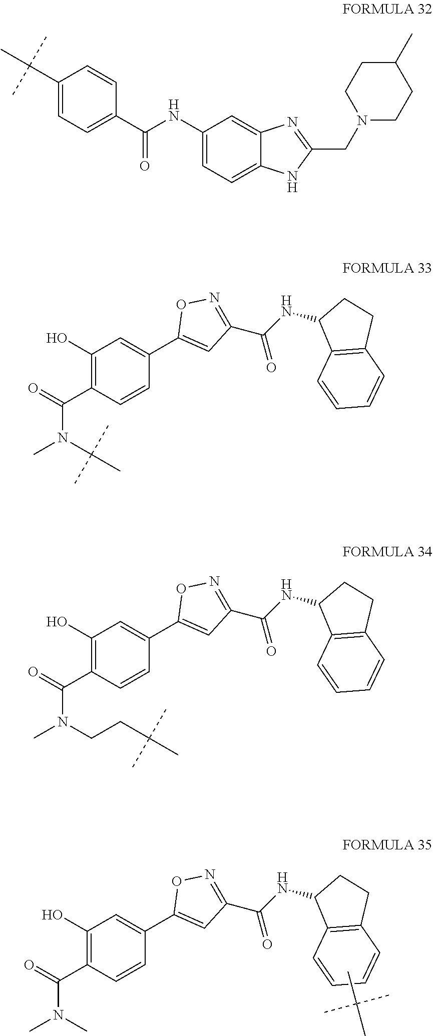

Degradation/Disruption Tags

Degradation/Disruption tags (EL) include, but are not limited to:

In an embodiment, degradation/disruption tags include a moiety according to FORMULAE 4A, 4B, 4C and 4D:

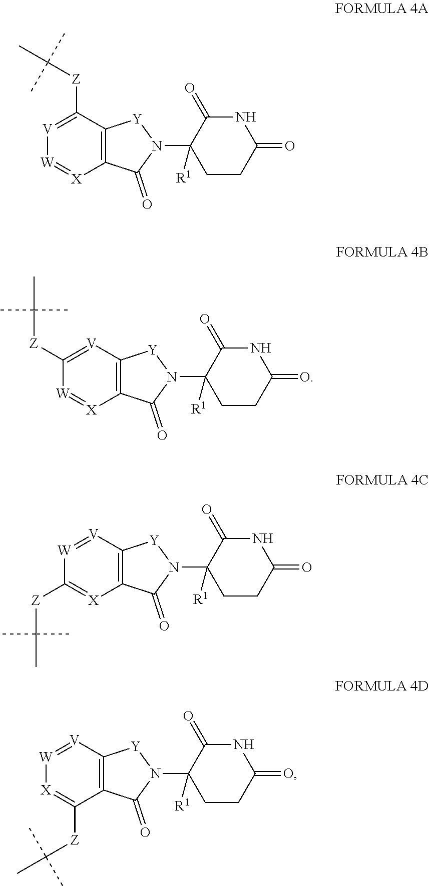

wherein
V, W, and X are independently selected from CR² and N;
Y is selected from CO, CR³R⁴, and N═N;
Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferly, Z is selected from null, CH₂, CH═CH, C≡C, NH and O;
R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;
R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and
R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl.

In an embodiment, degradation/disruption tags include a moiety according to one of FORMULAE 4E, 4F, 4G, 4H, and 4I:

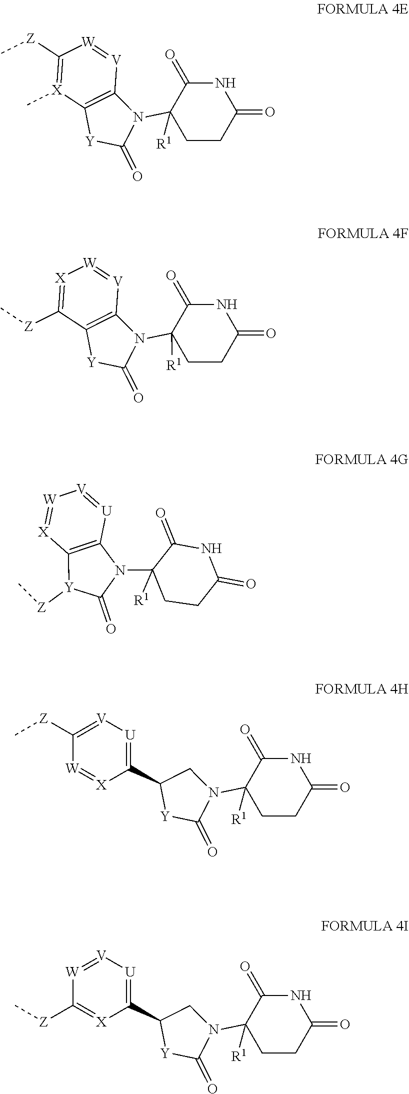

wherein
U, V, W, and X are independently selected from CR² and N;
Y is selected from CR³R⁴, NR³ and O; preferably, Y is selected from CH₂, NH, NCH₃ and O;
Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;
R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;
R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and
R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl; and
   pharmaceutically acceptable salts thereof.

In an embodiment, degradation/disruption tags include a moiety according to FORMULA 5A:

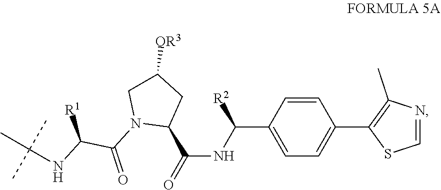

wherein

R¹ and R² are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₇ cycloalkyl, optionally substituted 3-7 membered heterocyclyl, optionally substituted C₂-C₈ alkenyl, and optionally substituted C₂-C₈ alkynyl; and

R³ is hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)C₃-C₇ cycloalkyl, optionally substituted C(O)(3-7 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)OC₃-C₇ cycloalkyl, optionally substituted C(O)O(3-7 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)NC₃-C₇ cycloalkyl, optionally substituted C(O)N(3-7 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂.

In an embodiment, degradation/disruption tags include a moiety according to FORMULAE 5B, 5C, 5D, 5E and 5F:

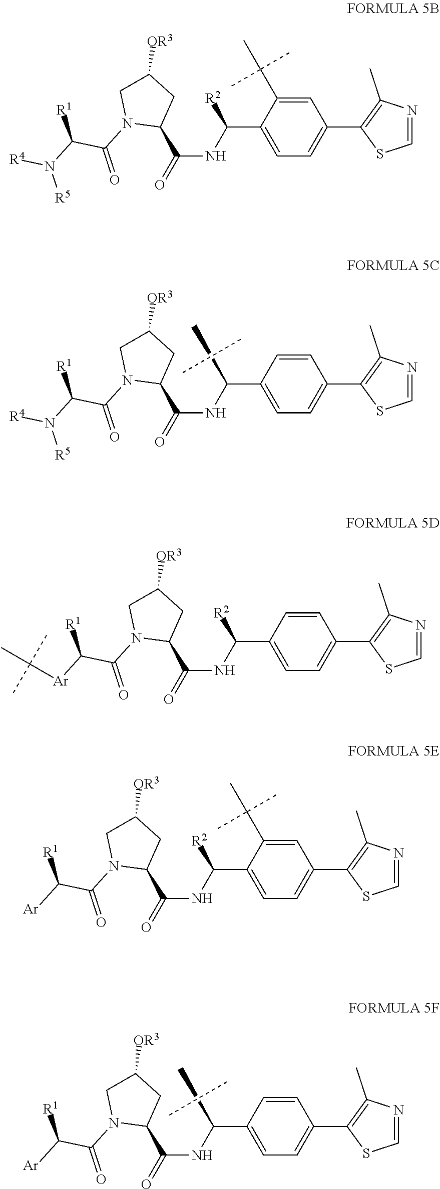

wherein
R¹ and R² are independently selected from hydrogen, halogen, OH, NH₂, CN, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₇ cycloalkyl, optionally substituted 3-7 membered heterocyclyl, optionally substituted C₂-C₈ alkenyl, and optionally substituted C₂-C₈ alkynyl; (preferably, R¹ is selected from iso-propyl or tert-butyl; and R² is selected from hydrogen or methyl);
R³ is hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)C₃-C₇ cycloalkyl, optionally substituted C(O)(3-7 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)OC₃-C₇ cycloalkyl, optionally substituted C(O)O(3-7 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)NC₃-C₇ cycloalkyl, optionally substituted C(O)N(3-7 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂; and
R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; wherein
R⁶ and R⁷ are independently selected from hydrogen, optionally substituted C₁-C₈ alkyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; or
R⁴ and R⁵; R⁶ and R⁷ together with the atom to which they are connected form a 4-8 membered cycloalkyl or heterocyclyl ring;
Ar is selected from aryl and heteroaryl, each of which is optionally substituted with one or more substituents independently selected from F, Cl, CN, NO₂, OR⁸, NR⁸R⁹, COR⁸, CO₂R⁸, CONR⁸R⁹, SOR⁸, SO₂R⁸, SO₂NR⁹R¹⁰, NR⁹COR¹⁰, NR⁸C(O)NR⁹R¹⁰, NR⁹SOR¹⁰, NR⁹SO₂R¹⁰, optionally substituted C₁-C₆ alkyl, optionally substituted C₁-C₆ alkoxyalkyl, optionally substituted C₁-C₆ haloalkyl, optionally substituted C₁-C₆ hydroxyalkyl, optionally substituted C₁-C₆alkylaminoC₁-C₆alkyl, optionally substituted C₃-C₇ cycloalkyl, optionally substituted 3-7 membered heterocyclyl, optionally substituted C₂-C₆ alkenyl, optionally substituted C₂-C₆ alkynyl, optionally substituted aryl, and optionally substituted C₄-C₅ heteroaryl; wherein
R⁸, R⁹, and R¹⁰ are independently selected from null, hydrogen, optionally substituted C₁-C₆ alkyl, optionally substituted C₂-C₆ alkenyl, optionally substituted C₂-C₆ alkynyl, optionally substituted C₃-C₇ cycloalkyl, optionally substituted 3-7 membered heterocyclyl, optionally substituted aryl, and optionally substituted heteroaryl; or
R⁸ and R⁹; R⁹ and R¹⁰ together with the atom to which they are connected form a 4-8 membered cycloalkyl or heterocyclyl ring; and
pharmaceutically acceptable salts thereof.

In an embodiment, degradation/disruption tags include a moiety according to FORMULA 5A:

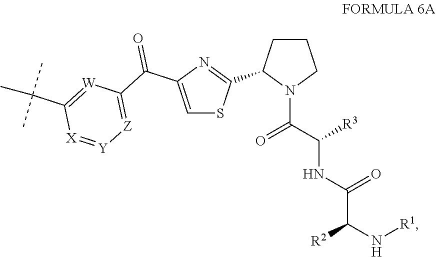

wherein
V, W, X, and Z are independently selected from CR⁴ and N;

R¹, R², R³, and R⁴ are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl.

In an embodiment, degradation/disruption tags include a moiety according to FORMULA 5B:

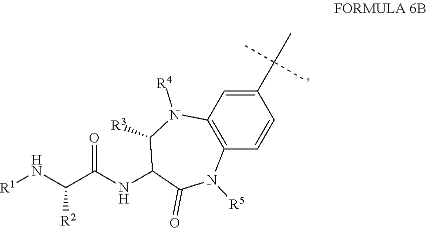

wherein
R¹, R², and R³ are independently selected from hydrogen, halogene, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl;
   R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted aryl-C₁-C₈alkyl, optionally substituted 3-8 membered cycloalkyl, optionally substituted 3-8 membered heterocyclyl, optionally substituted aryl, and optionally substituted heteroaryl; wherein
   R⁶ and R⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; or
   R⁶ and R⁷ together with the atom to which they are connected form a 4-8 membered cycloalkyl or heterocyclyl ring; and
pharmaceutically acceptable salts thereof.

In an embodiment, degradation/disruption tags are selected from the group consisting of:

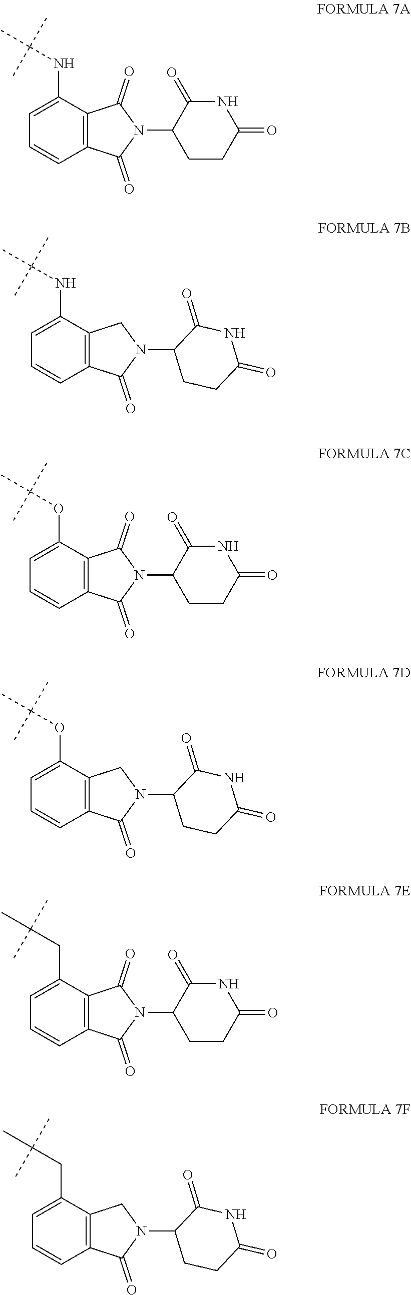

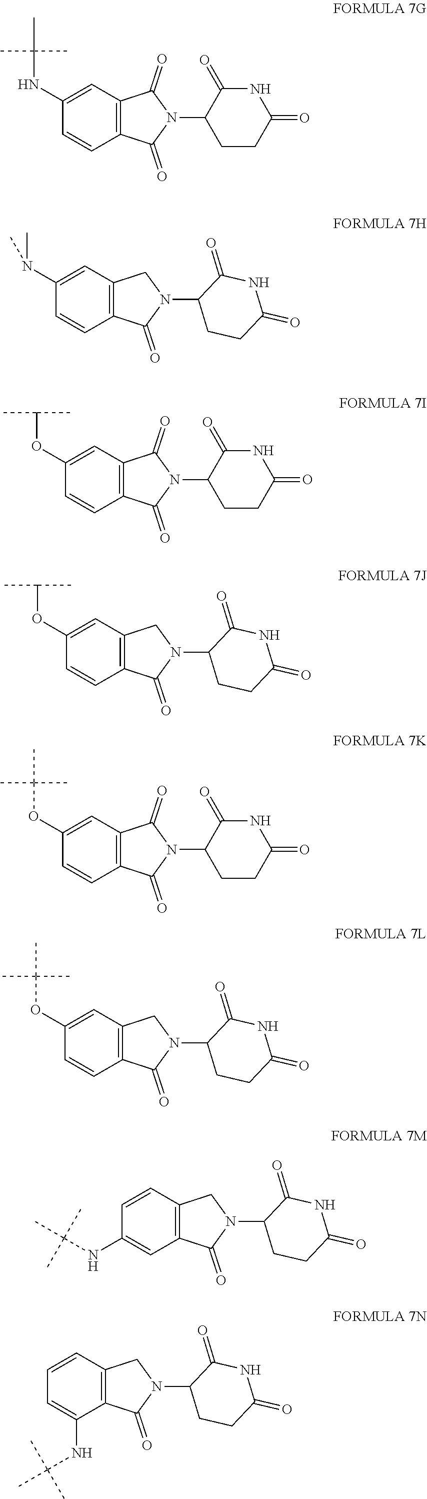

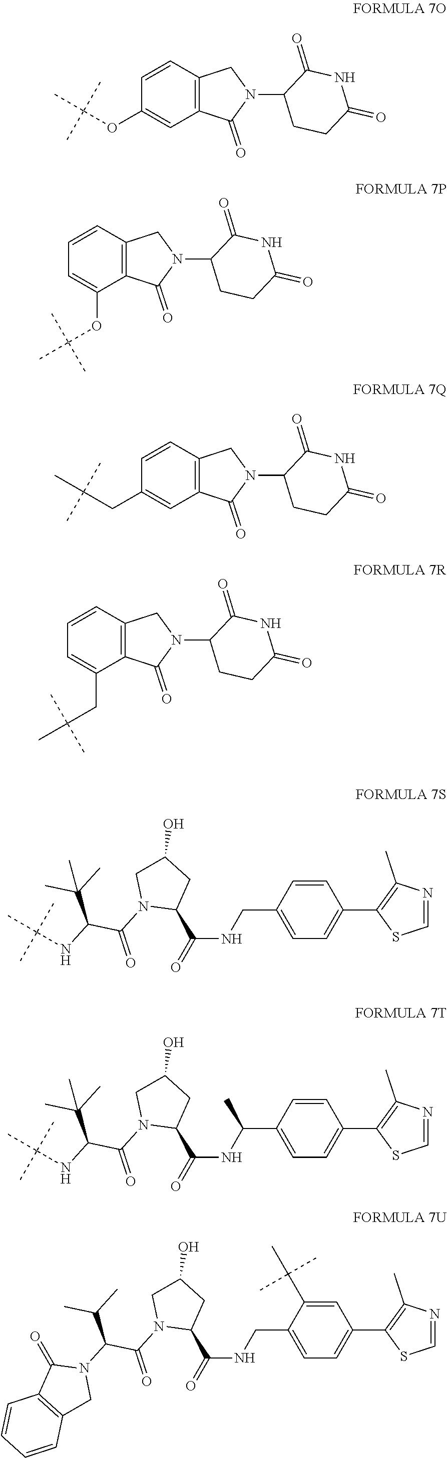

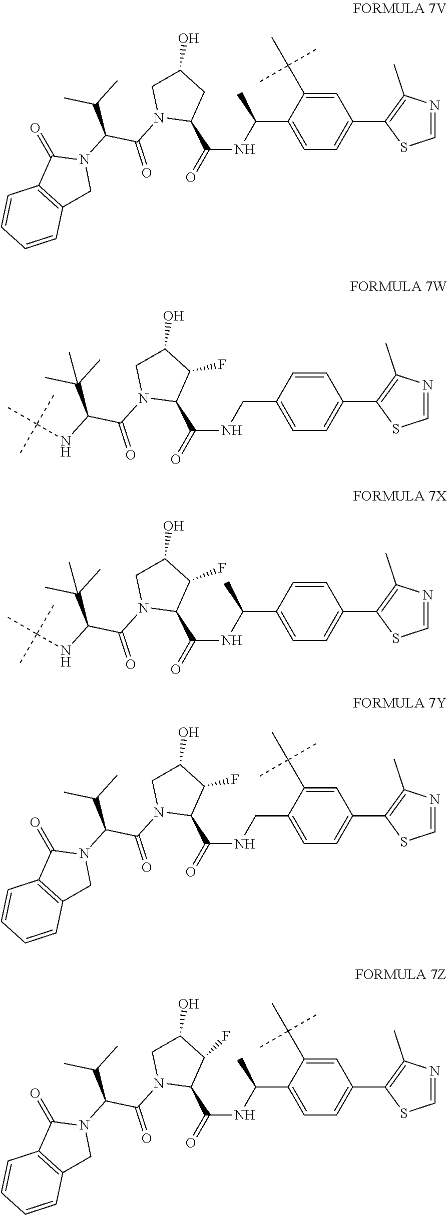

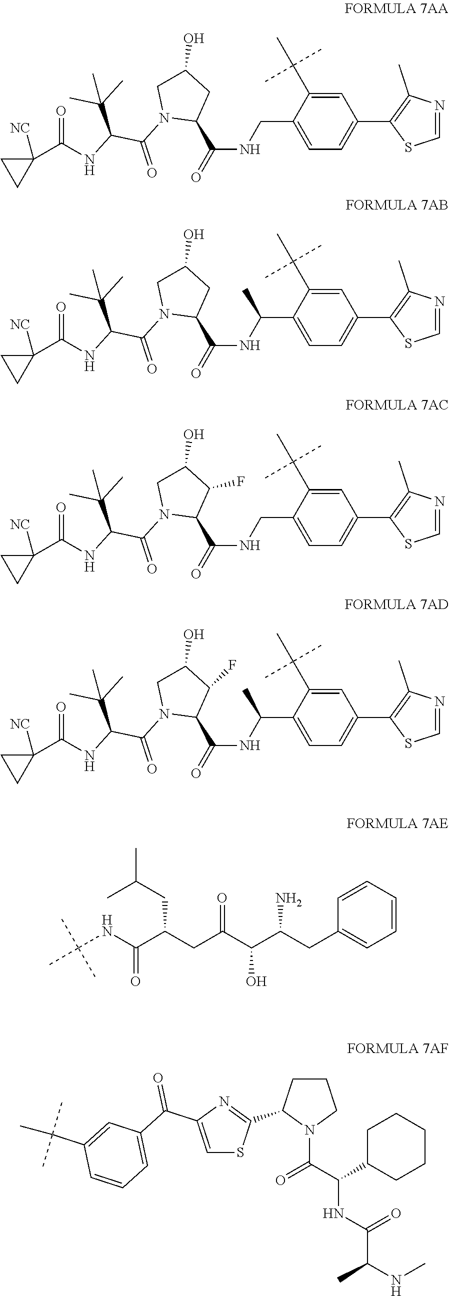

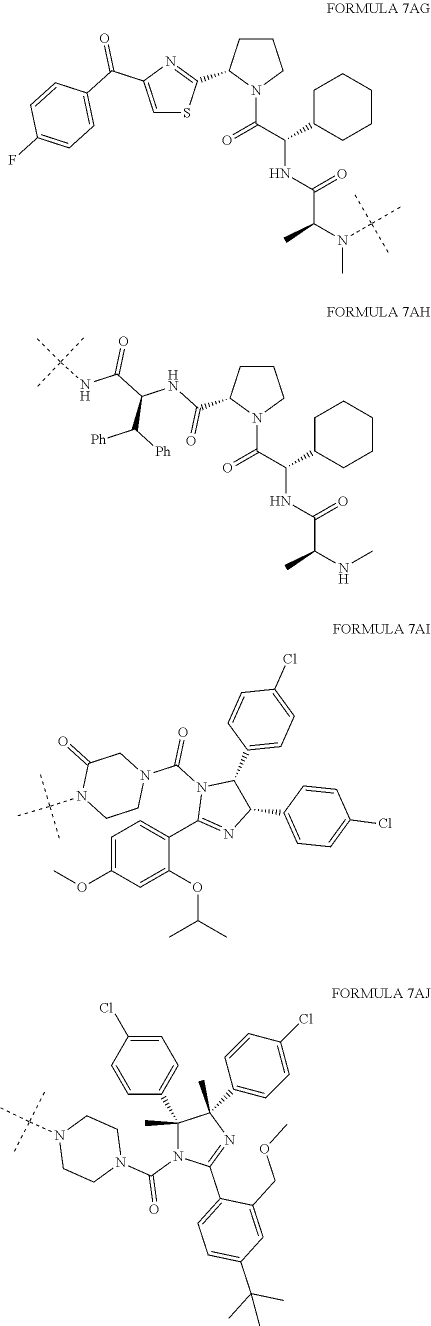

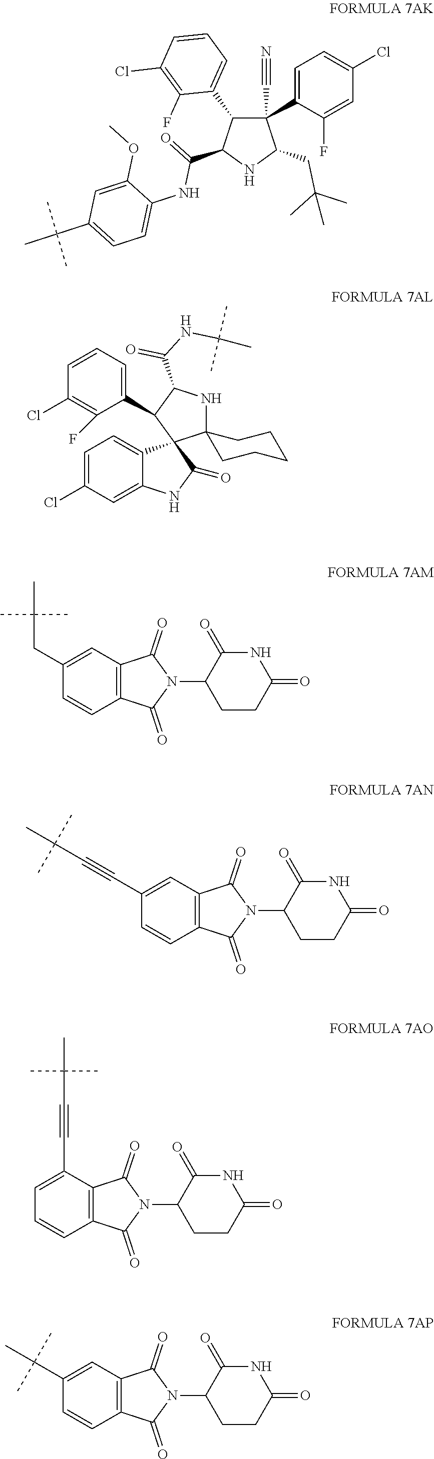

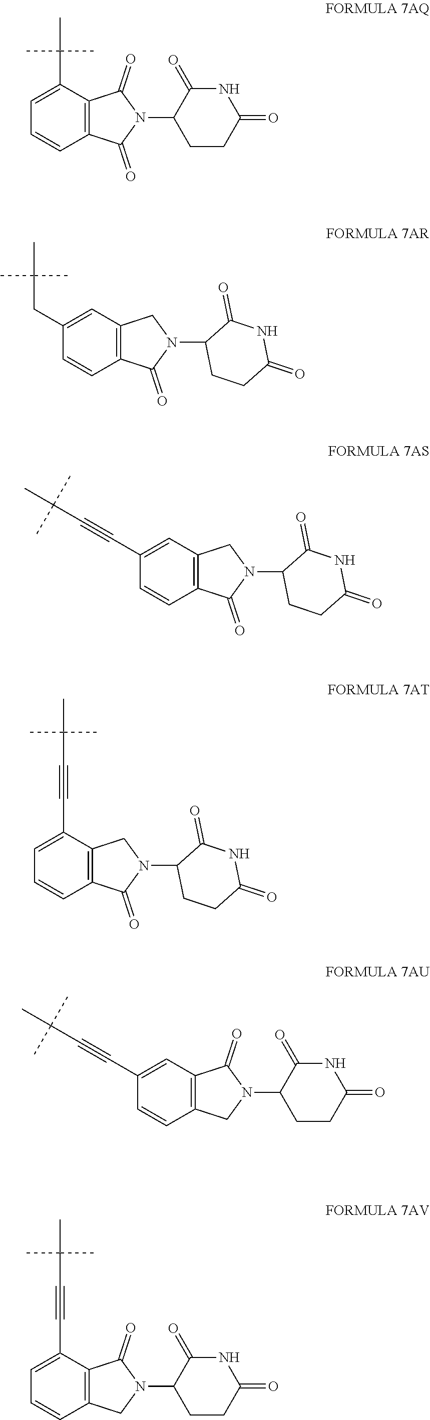

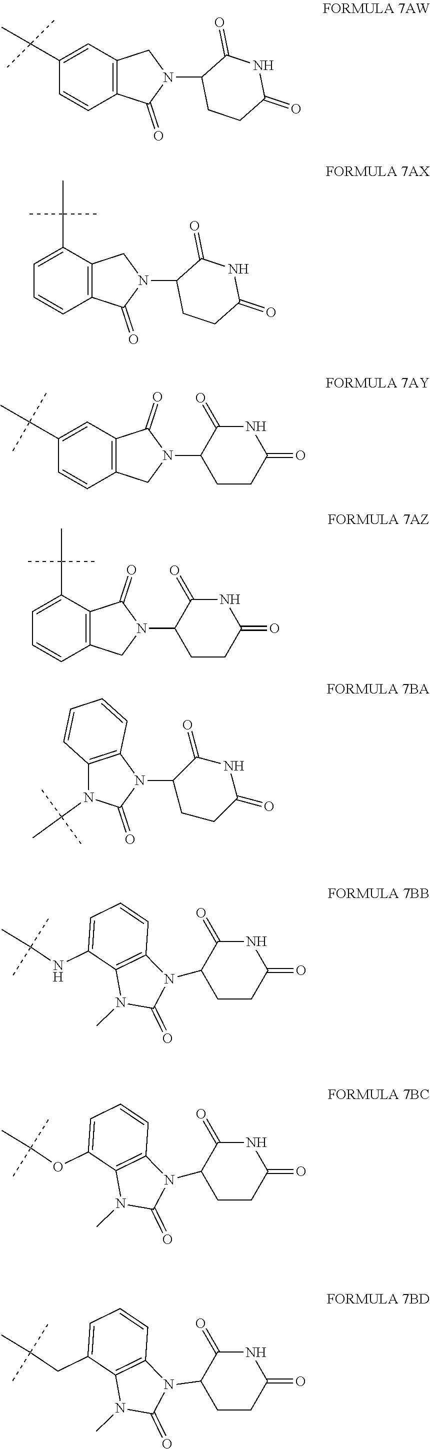

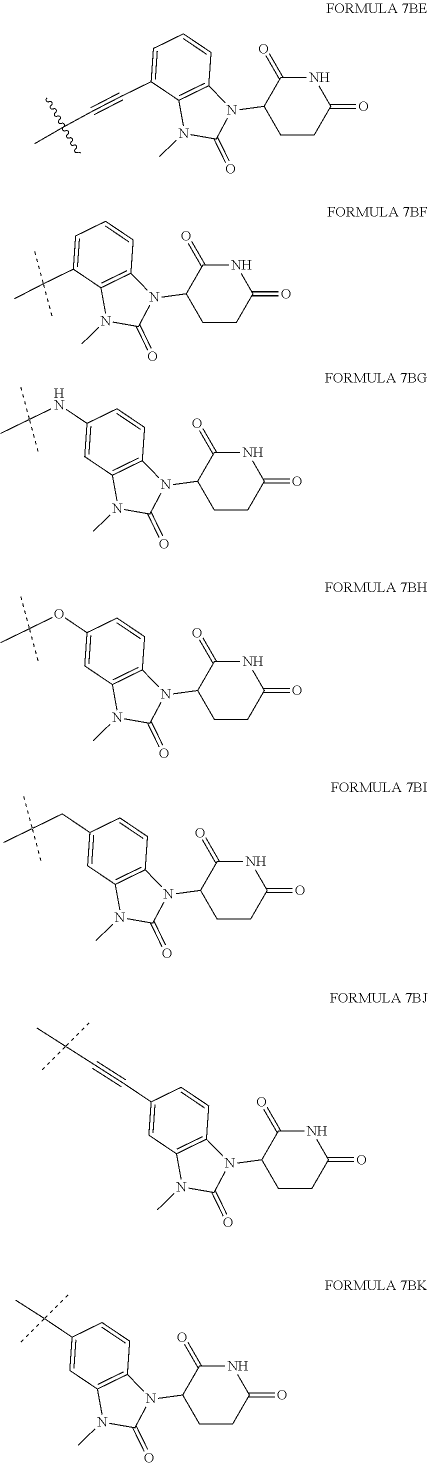

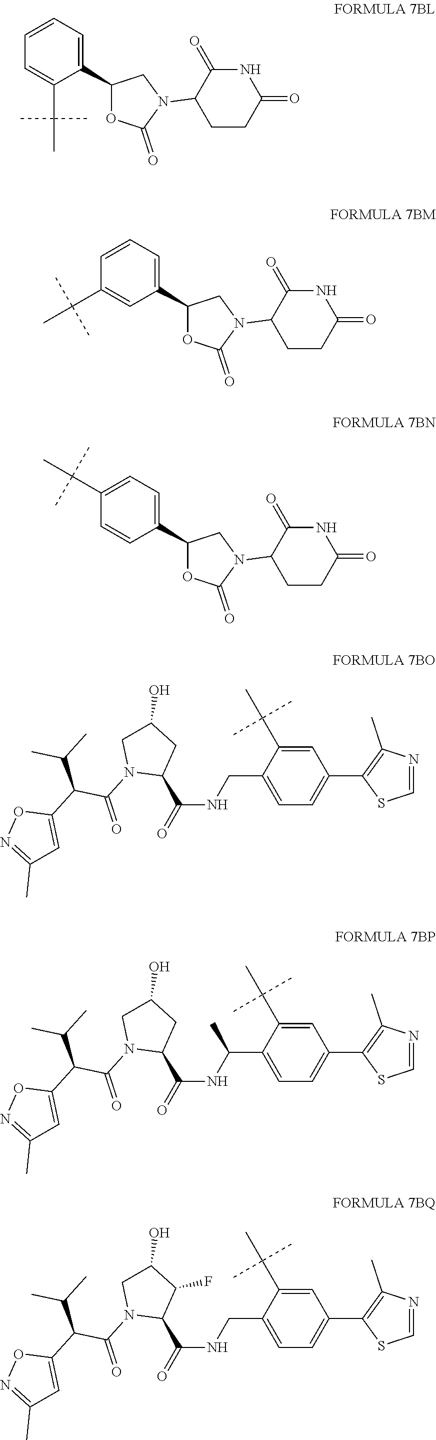

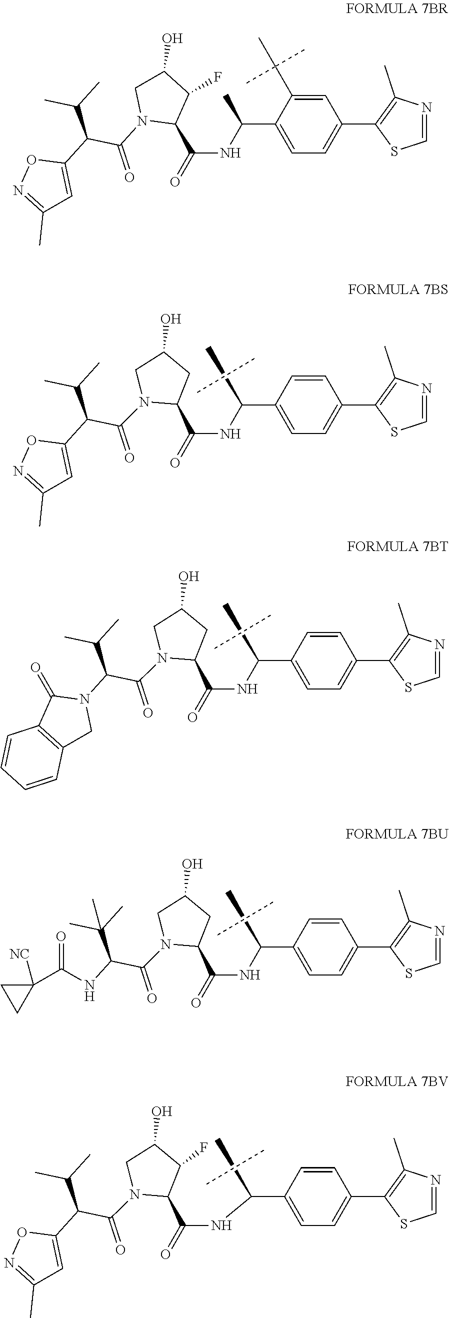

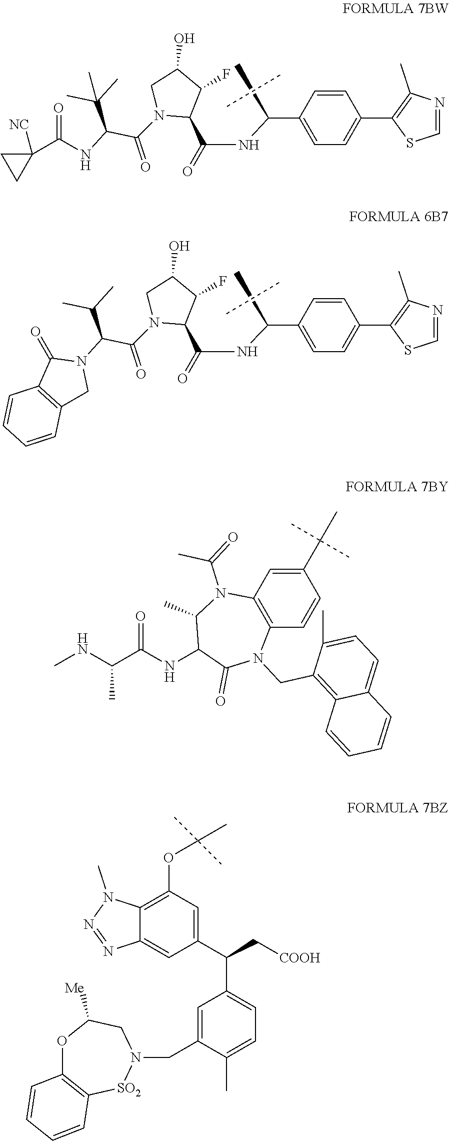

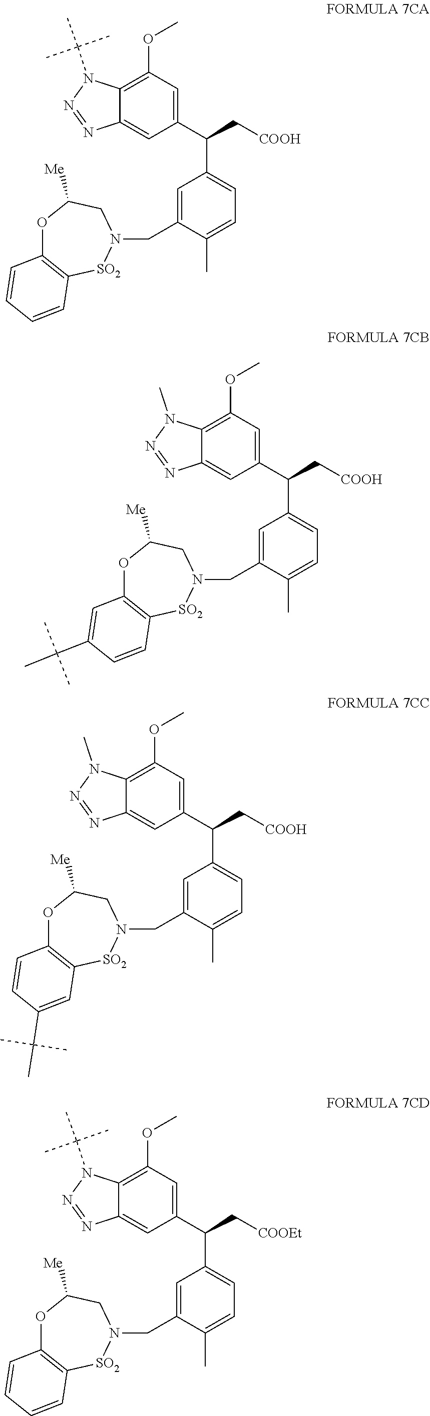

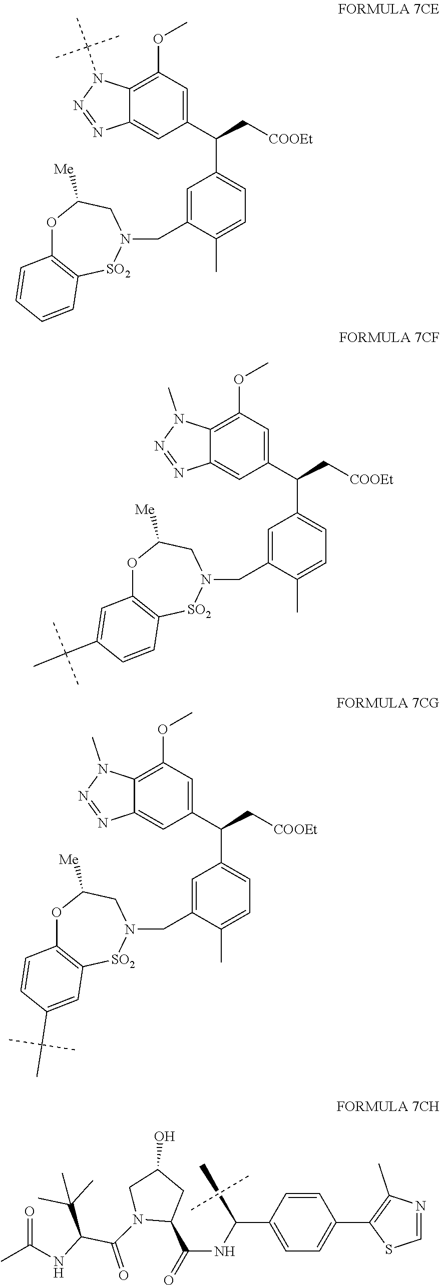

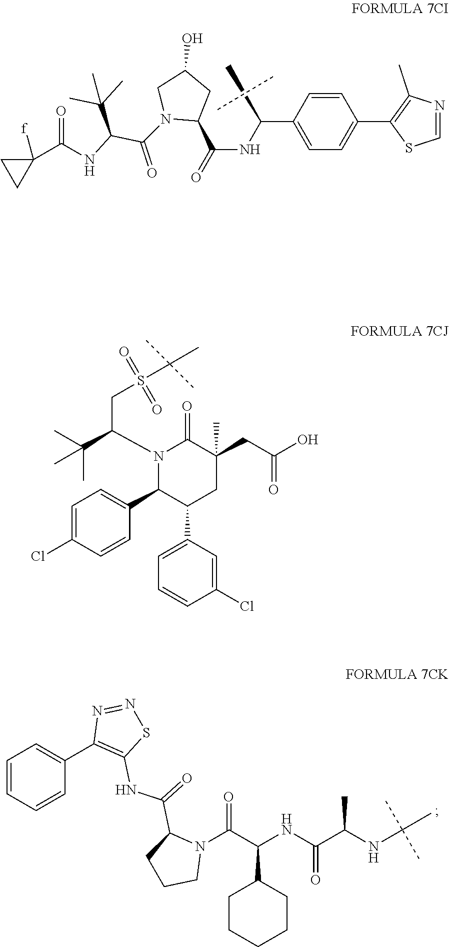

and pharmaceutically acceptable salts thereof.

### Linkers

In any of the above-described compounds, the ENL ligand can be conjugated to the degradation/disruption tag through a linker. The linker can include, e.g., acyclic or cyclic saturated or unsaturated carbon, ethylene glycol, amide, amino, ether, urea, carbamate, aromatic, heteroaromatic, heterocyclic, and/or carbonyl containing groups with different lengths.

In an embodiment, the linker is a moiety according to FORMULA 8:

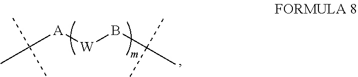

wherein
A, W, and B, at each occurrence, are independently selected from null, CO, CO₂, C(O)NR¹, C(S)NR¹, O, S, SO, SO₂, SO₂NR¹, NR¹, NR¹CO, NR¹CONR², NR¹C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R¹ and R² are independently selected from hydrogen, optionally substituted C₁-C₈ alkyl, optionally substituted C₃-C₈ cycloalkyl, optionally substituted 3-8 membered cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈alkylaminoC₁-C₈alkyl; and
m is 0 to 15.

In an embodiment, the linker is a moiety according to FORMULA 8A:

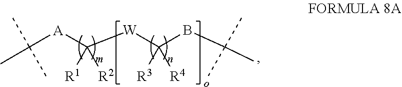

wherein
R¹, R², R³, and R⁴, at each occurrence, are independently selected from hydrogen, halogen, CN, OH, NH₂, optionally substituted C₁-C₈ alkyl, optionally substituted C₃-C₈ cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
A, W, and B, at each occurrence, are independently selected from null, CO, CO₂, C(O)NR⁵, C(S)NR⁵, O, S, SO, SO₂, SO₂NR⁵, NR⁵, NR⁵CO, NR⁵CONR⁶, NR⁵C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
   R⁵ and R⁶ are independently selected from hydrogen, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈alkylaminoC₁-C₈alkyl;
m is 0 to 15;
n, at each occurrence, is 0 to 15;
o is 0 to 15.

In an embodiment, the linker is a moiety according to FORMULA 8B:

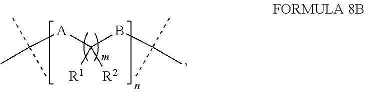

wherein
R¹ and R², at each occurrence, are independently selected from hydrogen, halogen, CN, OH, NH₂, and optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, or C₁-C₈alkylaminoC₁-C₈alkyl;
A and B, at each occurrence, are independently selected from null, CO, CO₂, C(O)NR³, C(S)NR³, O, S, SO, SO₂, SO₂NR³, NR³, NR³CO, NR³CONR⁴, NR³C(S), and optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, or C₃-C₁₃ spiro heterocyclyl; wherein
R³ and R⁴ are independently selected from hydrogen, and optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, or C₁-C₈alkylaminoC₁-C₈alkyl;
each m is 0 to 15; and
n is 0 to 15.

In an embodiment, the linker is a moiety according to FORMULA 8C:

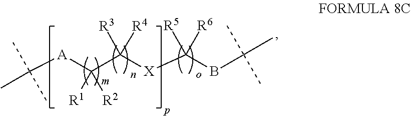

wherein
X is selected from 0, NH, and NR⁷;
R¹, R², R³, R⁴, R⁵, and R⁶, at each occurrence, are independently selected from hydrogen, halogen, CN, OH, NH₂, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH—CH₂—NH—CH₂, CH₂—NH—CH₂, CO₂, C(O)NR⁷, C(S)NR⁷, O, S, SO, SO₂, SO₂NR⁷, NR⁷, NR⁷CO, NR⁷CONR⁸, NR⁷C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R⁷ and R⁸ are independently selected from hydrogen, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
m, at each occurrence, is 0 to 15;
n, at each occurrence, is 0 to 15;
o is 0 to 15; and
p is 0 to 15; and
pharmaceutically acceptable salts thereof.

In an embodiment, the linker is selected from the group consisting of a ring selected from the group consisting of a 3 to 13 membered ring; a 3 to 13 membered fused ring; a 3 to 13 membered bridged ring; and a 3 to 13 membered spiro ring; and pharmaceutically acceptable salts thereof.

In an embodiment, the linker is a moiety according to one of FORMULAE C1, C2, C3, C4 and C5.

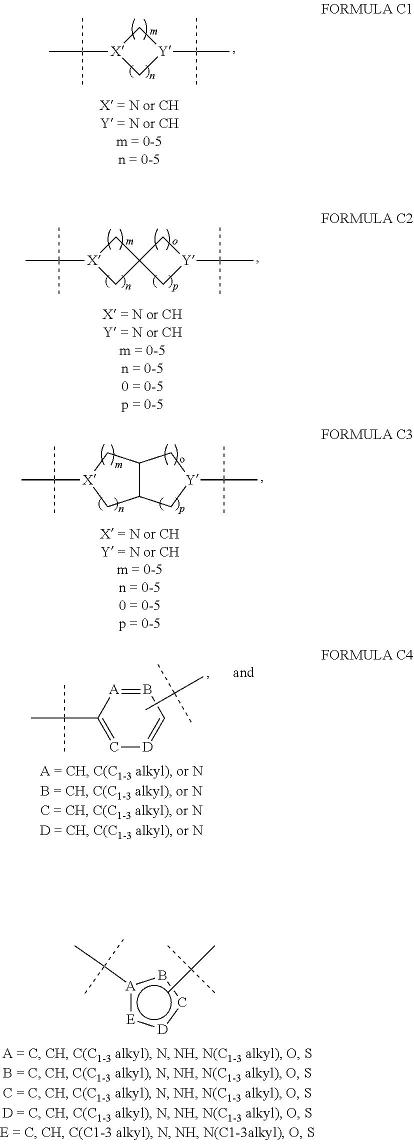

FORMULA C5; and pharmaceutically acceptable salts thereof.

### Synthesis and Testing of Bivalent Compounds

The binding affinity of novel synthesized bivalent compounds (i.e., ENL degraders/disruptors) can be assessed using standard biophysical assays known in the art (e.g., isothermal titration calorimetry (ITC)). Cellular assays can then be used to assess the bivalent compound's ability to induce ENL degradation and inhibit cancer cell proliferation. Suitable cell lines for use in any or all of these steps are known in the art and include, e.g. MV4; 11, Jurkat, MOLM13. Suitable mouse models for use in any or all of these steps are known in the art and include MV4; 11 and MOLM13 xenograft model.

By way of non-limiting example, detailed synthesis protocols are described in the Examples for specific exemplary ENL degraders/disruptors.

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹⁵N, ¹⁷O, ¹⁸O, ³²P, ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements. In addition, certain isotopic variations (particularly those containing a radioactive isotope) can be used in drug or substrate tissue distribution studies. The radioactive isotopes tritium (³H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, d₆-acetone in place of acetone, or d₆-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements

Pharmaceutically acceptable prodrugs of the compounds disclosed herein are contemplated and can be synthesized using conventional methods known in the art or methods corresponding to those described in the Examples (e.g., concerting hydroxyl groups to ester groups or sodium phosphate salt). As used herein, a “prodrug” refers to a compound that can be converted via some chemical or physiological process (e.g., enzymatic process and metabolic hydrolysis) to a therapeutic agent. Thus, the term “prodrug” also refers to a precursor of a biologically active compound that is pharmaceutically acceptable. A prodrug may be inactive when administered to a subject, but is converted in vivo to an active compound. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively.

### Characterization of Exemplary ENL Degraders/Disruptors

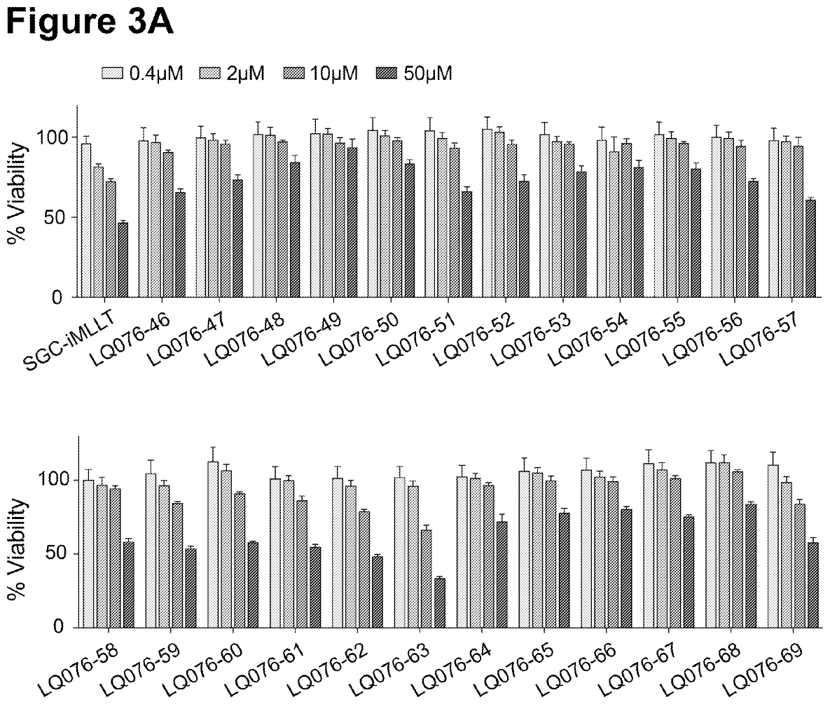

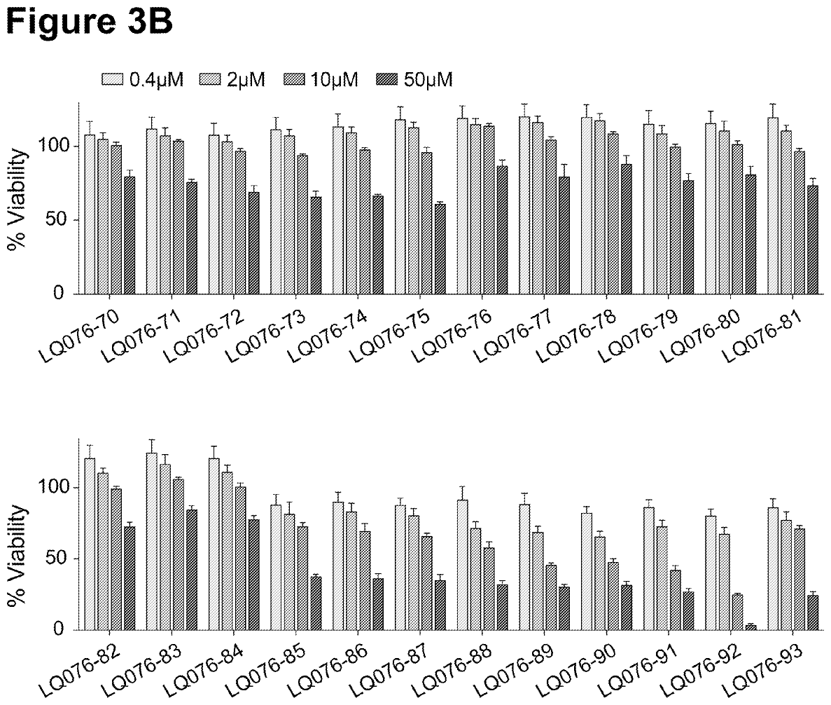

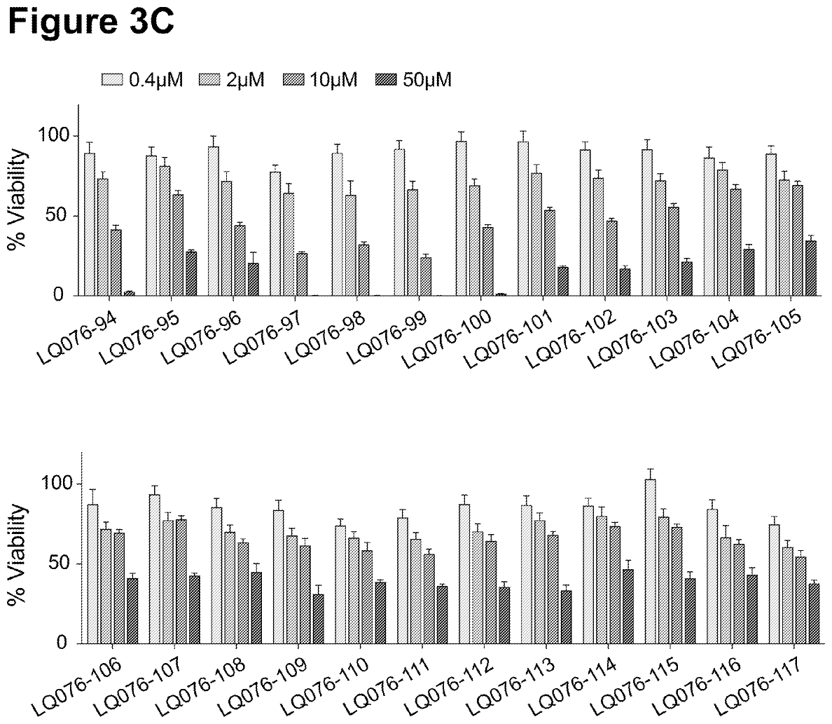

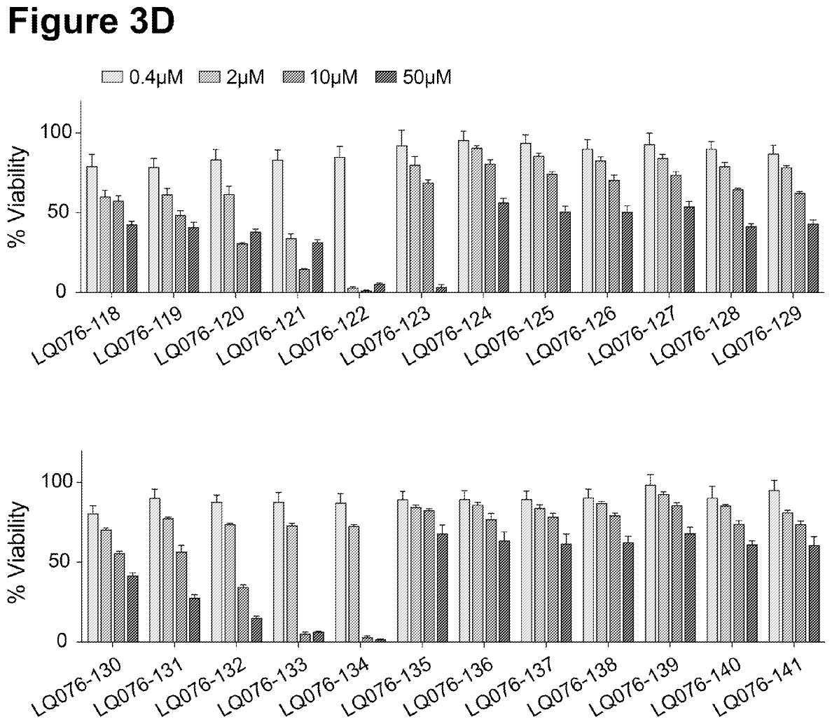

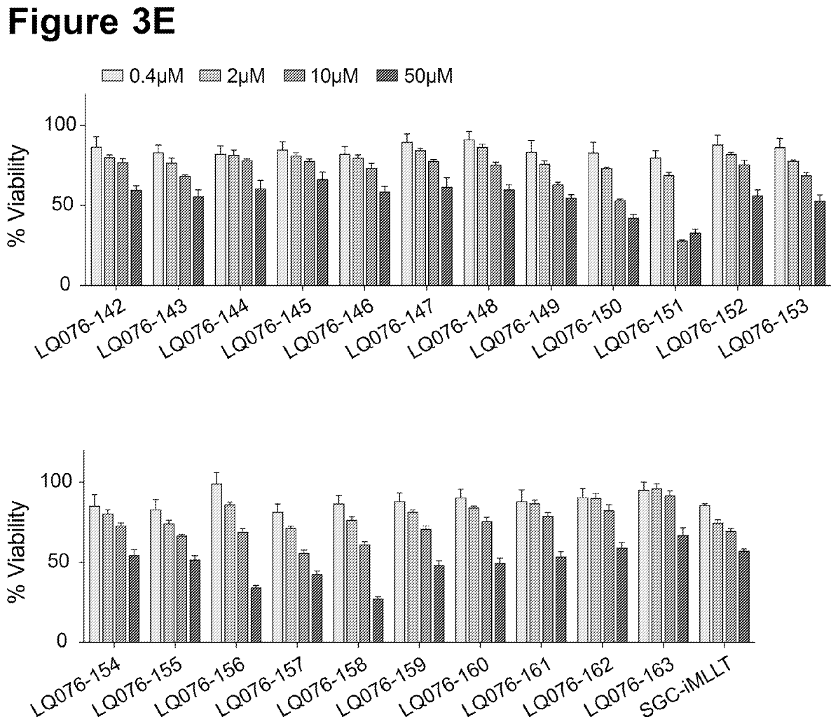

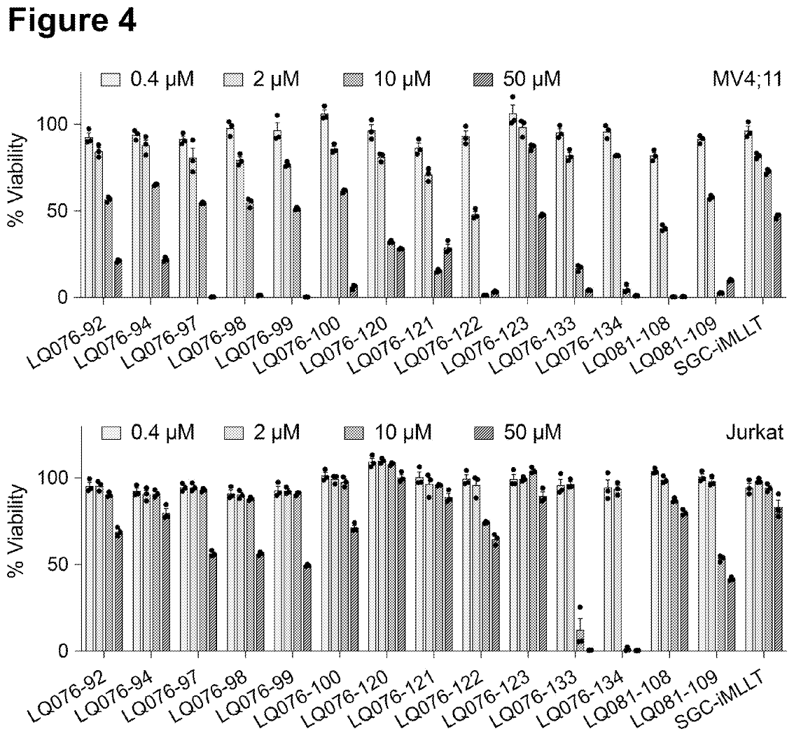

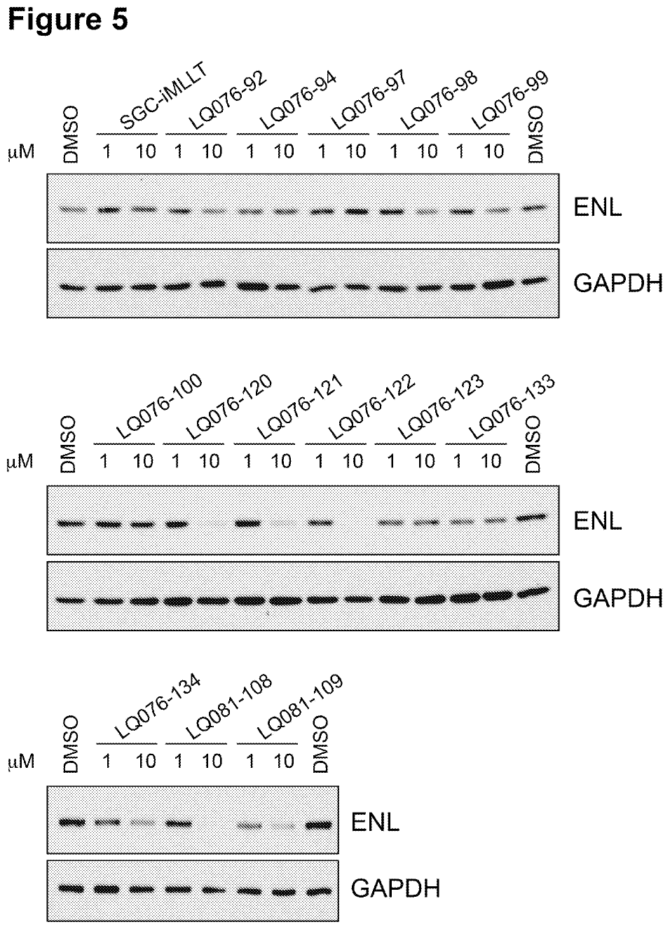

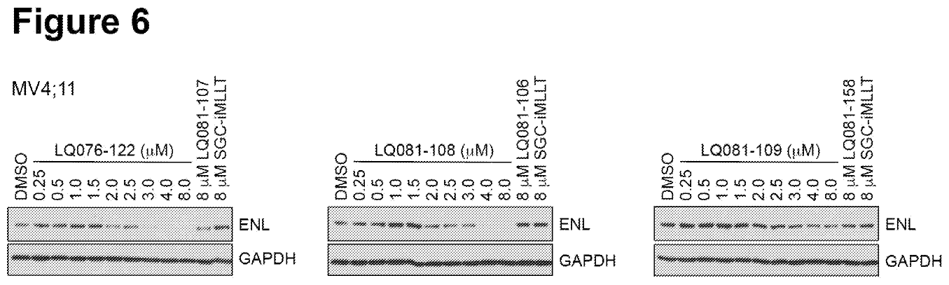

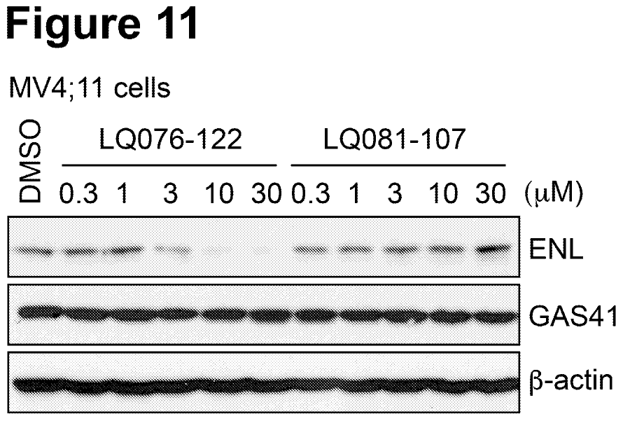

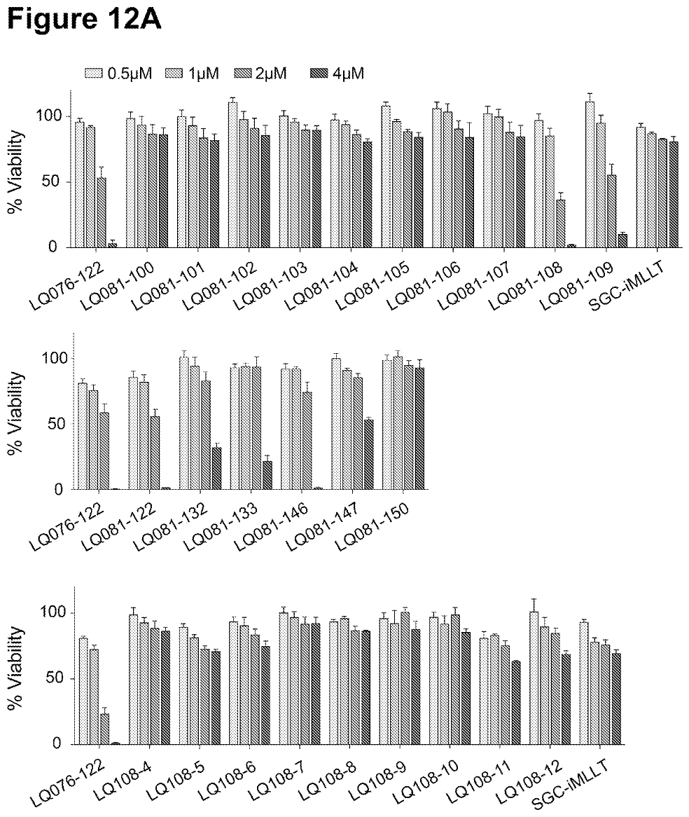

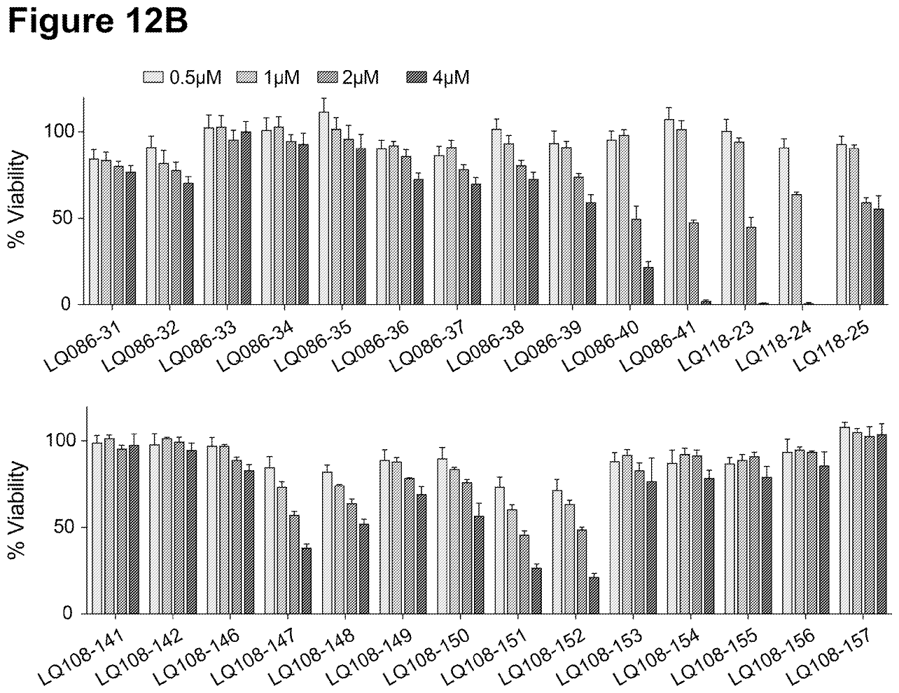

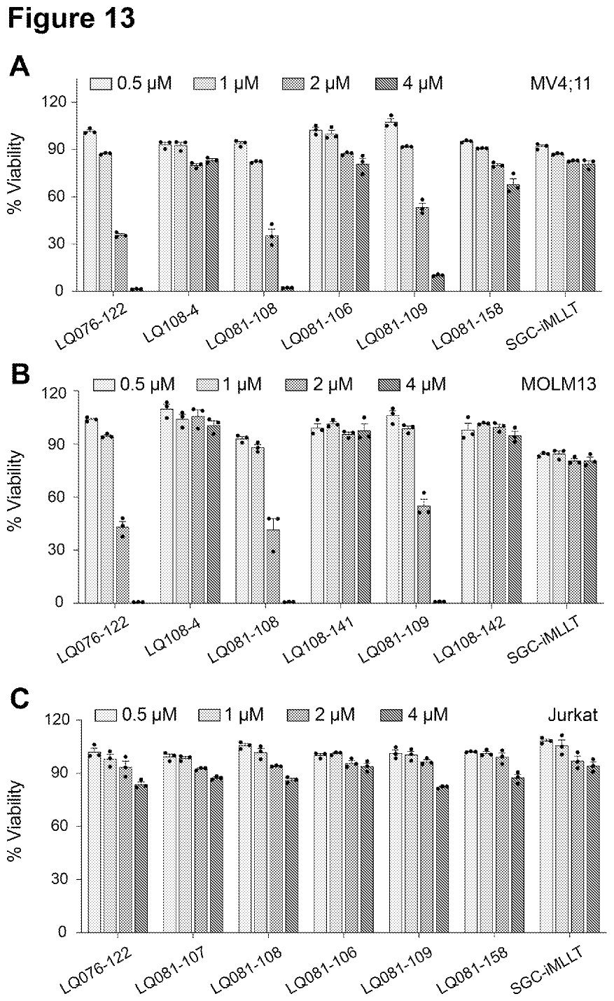

Specific exemplary ENL degraders/disruptors were firstly characterized in ENL-dependent leukemia MV4; 11 cells to evaluate their concentration-dependent ability in cell growth suppression (FIG. 3 and FIG. 12). Compounds achieved >50% cell growth inhibition at 10 μM in MV4; 11 cells were further characterized in an ENL-independent leukemia cell lines Jurkat (FIG. 4). The same panel of compounds were tested by Western blotting for their efficiencies in reducing ENL protein levels in MV4; 11 cells at 1 μM and 10 μM. Bifunctional compounds LQ076-98, LQ076-99, LQ076-120, LQ076-121, LQ076-122, LQ076-134, LQ081-108 and LQ081-109 were identified to be effective in reducing ENL protein levels in MV4; 11 cells at 10 μM (FIG. 5). In particular, LQ076-122, LQ081-108 and LQ081-109 were found effective in a concentration- and time-dependent manner while the non-degrader ENL inhibitor SGC-iMLLT had no effect on reducing ENL protein levels (FIG. 6-10). In addition, LQ076-122 showed no effect on other YEATS domain-containing proteins, such as GAS41 (FIG. 11). LQ076-122, LQ081-108 and LQ081-109 significantly suppressed MV4; 11 and MOLM13 cell growth at low micromolar concentration, but did not affect Jurkat cells, phenocoping the results seen in ENL knockout cells (FIG. 13).

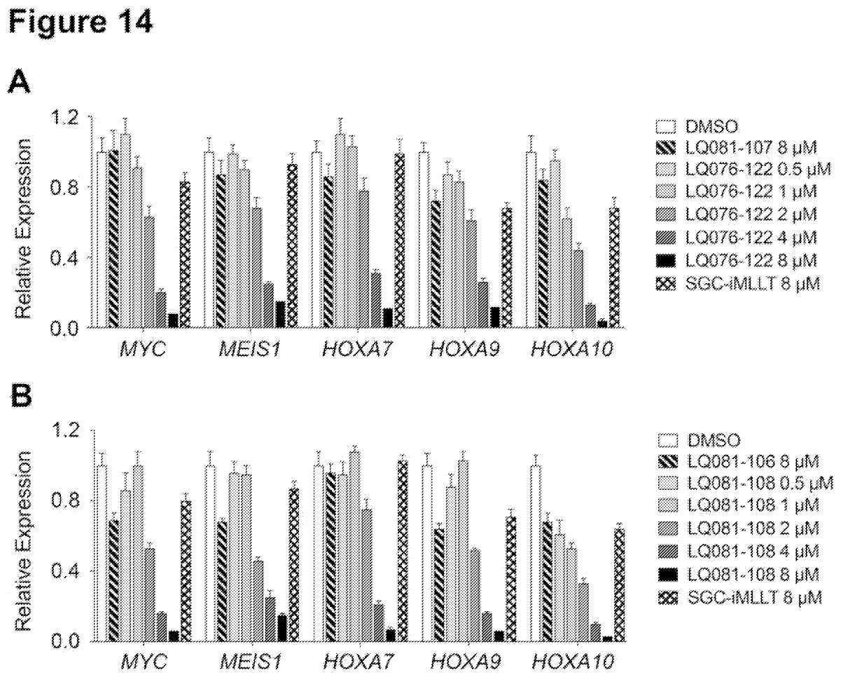

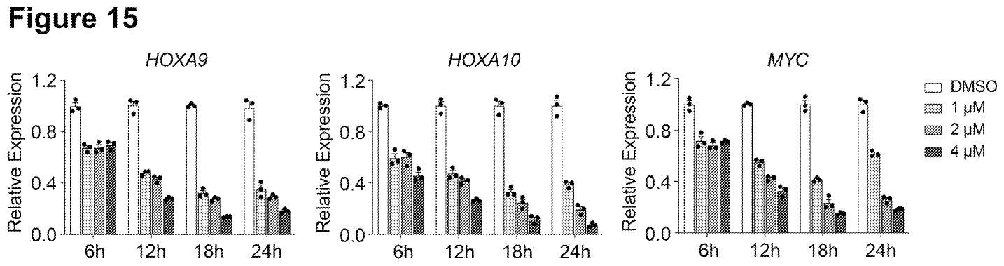

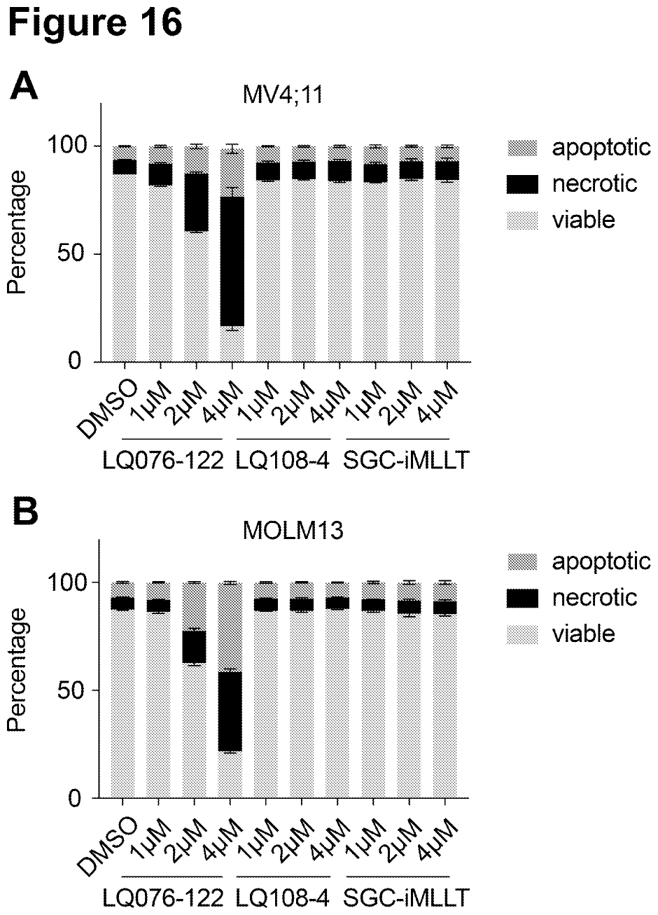

Treatment of cells with ENL degraders LQ076-122 and LQ081-108 suppressed ENL target gene expression in a concentration- and time-dependent manner in both MOLM13 and MV4; 11 cells (FIG. 14-15). Neither ENL inhibitor SGC-iMLLT nor negative control compounds showed an effective suppression of ENL target gene expression (FIG. 14). Treatment of cells with LQ076-122 induced apoptosis in MV4; 11 and MOLM13 cells, which was not observed in cells treated with SGC-iMLLT or negative control compound (FIG. 16).

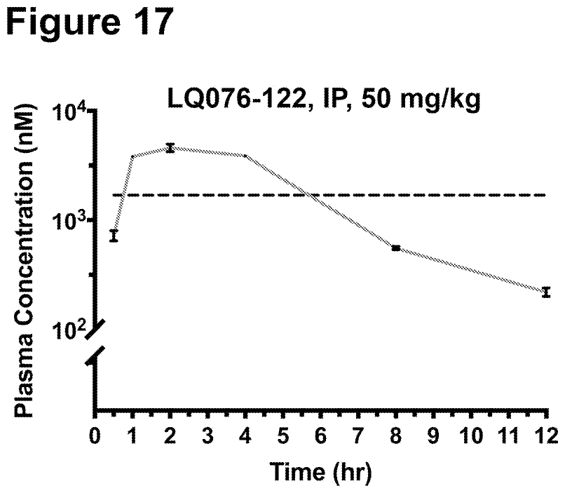

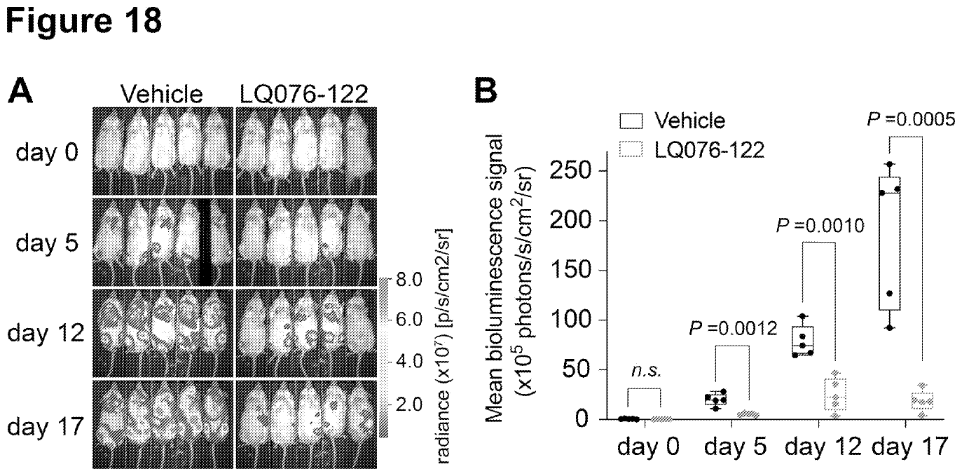

The plasma concentrations of ENL degrader LQ076-122 was measured over 12 h following a single 50 mg/kg IP injection in a mouse pharmacokinetic (PK) study. The concentrations of LQ076-122 in plasma were maintained above 2 μM for 6 h with the maximum plasma concentration of about 6 μM (FIG. 17). In a xenograft study where immuno-deficient NSG mice were transplanted with MV4; 11-Luc cells through intravenous xenograft, three cycles of LQ076-122 treatment significantly inhibited leukemia progression (FIG. 18), highlighting the potential utility of ENL degraders for ENL-dependent cancer treatment.

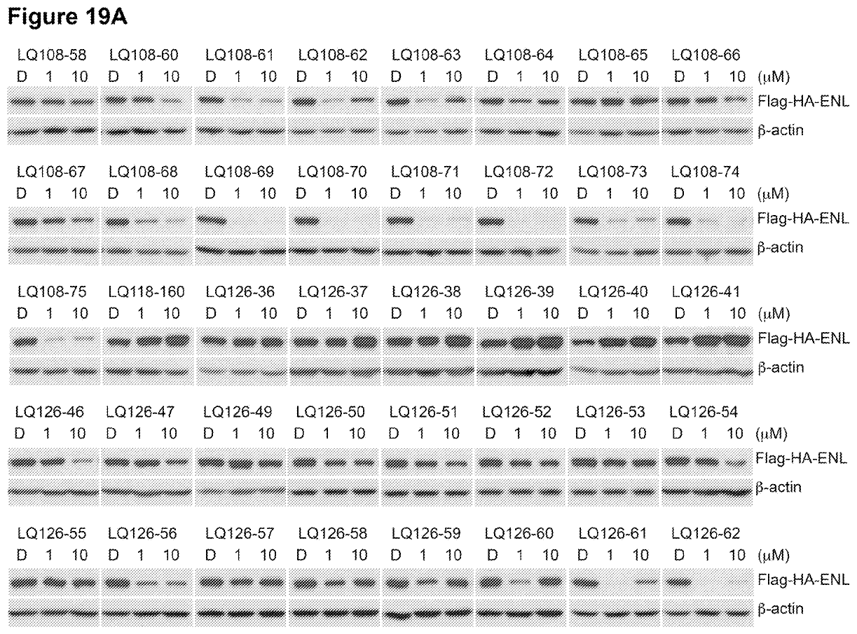

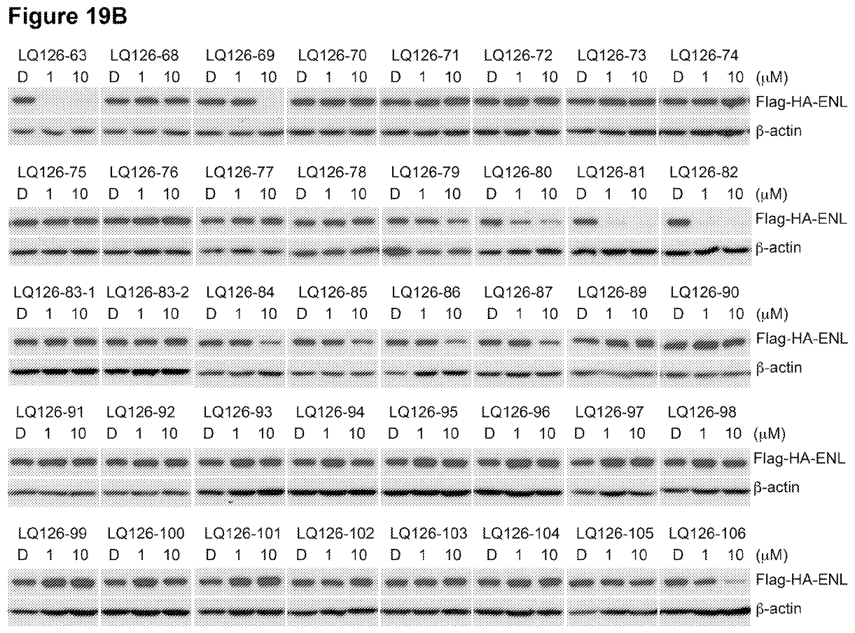

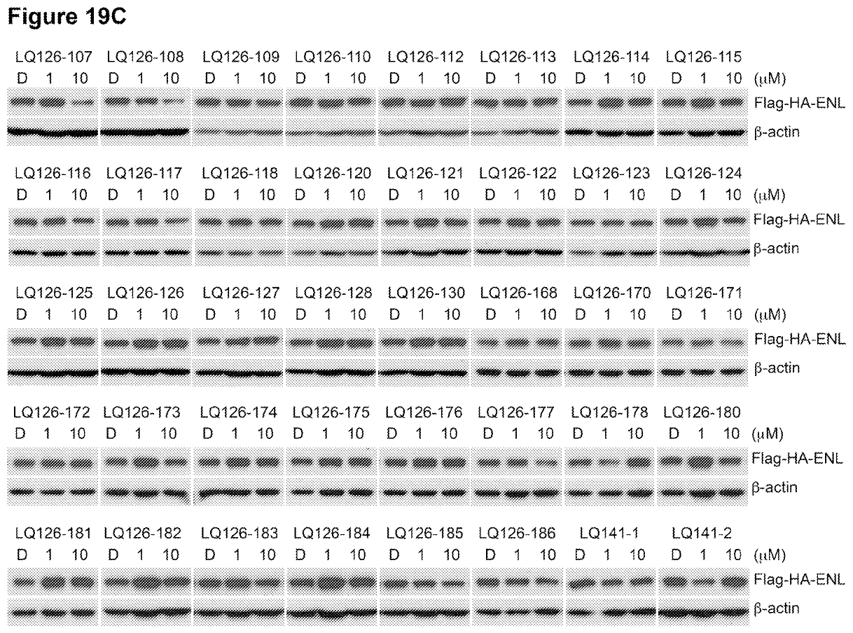

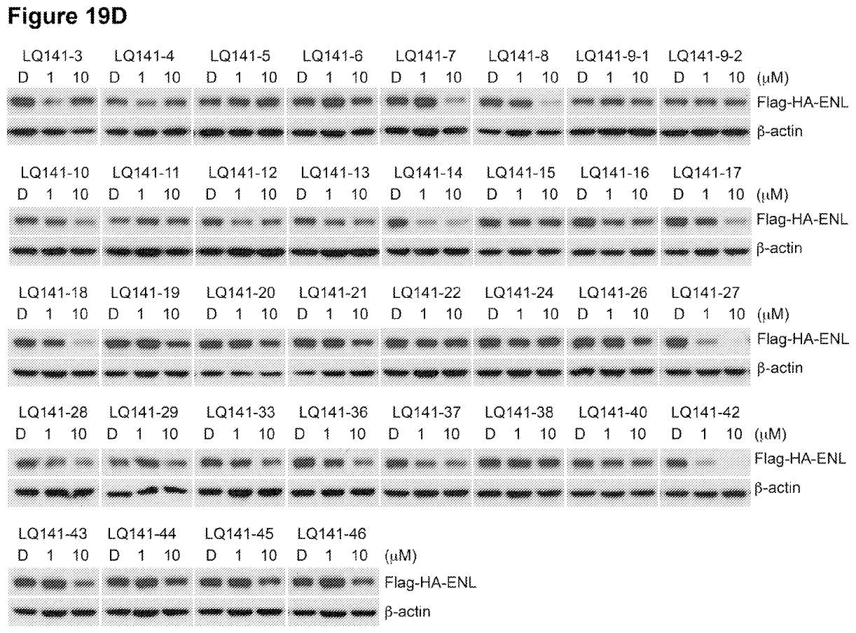

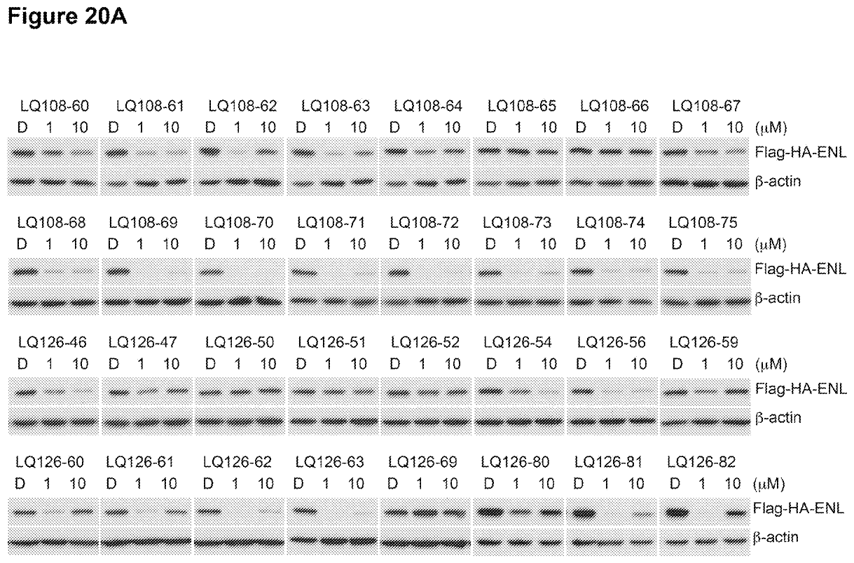

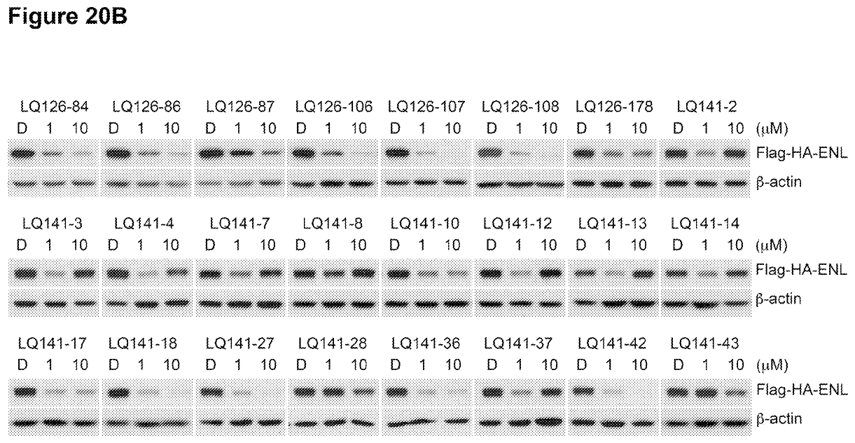

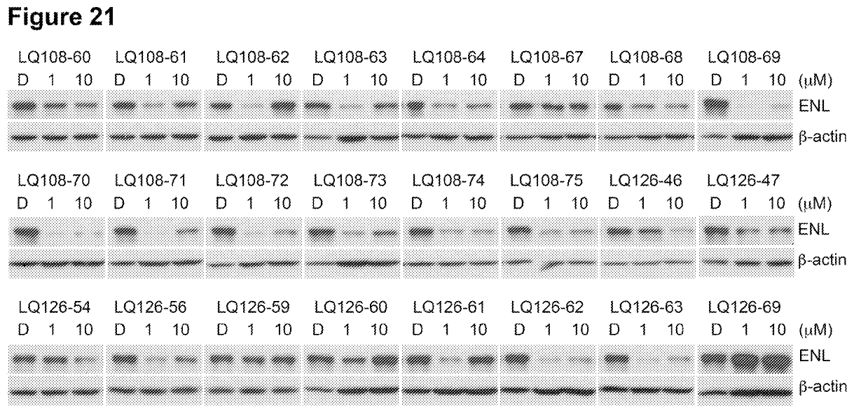

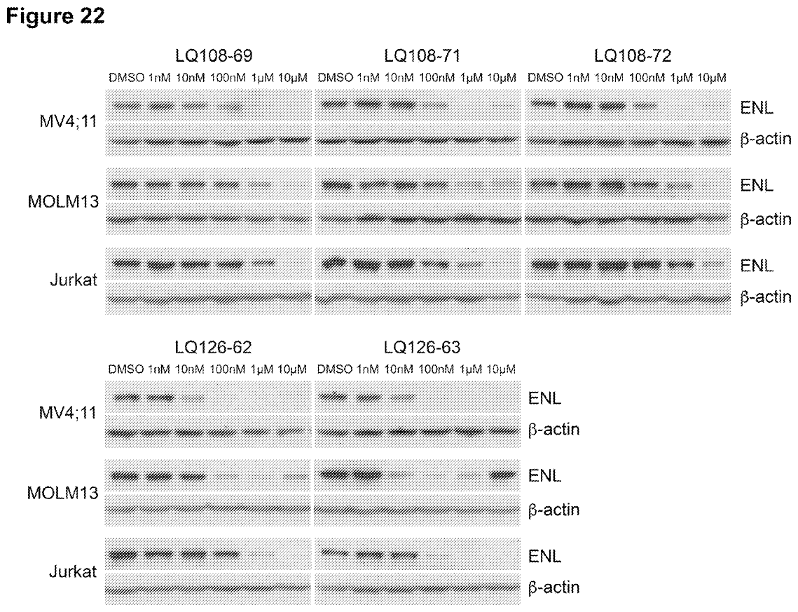

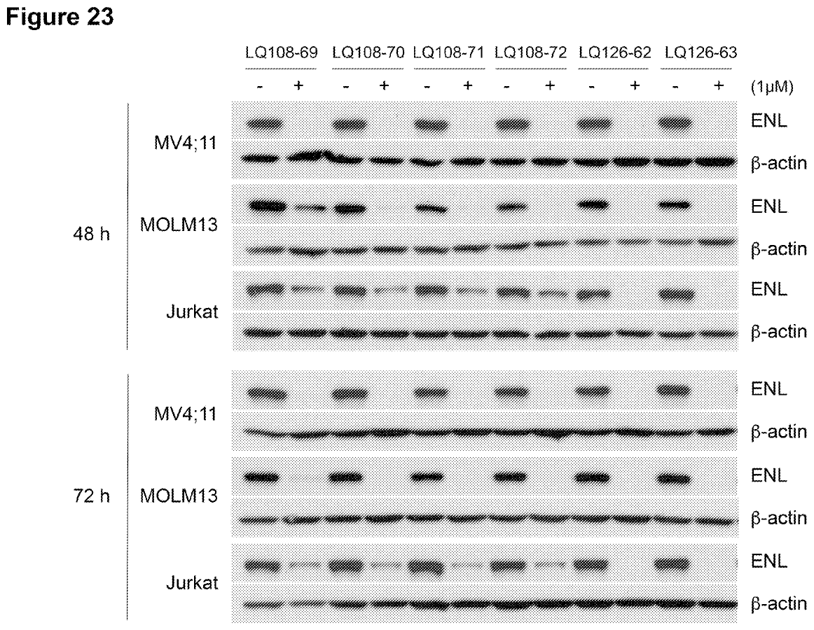

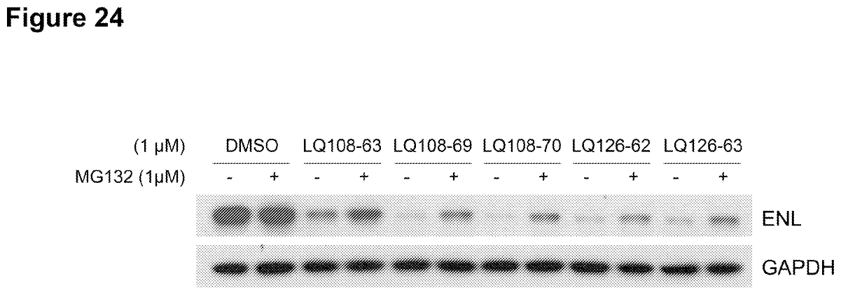

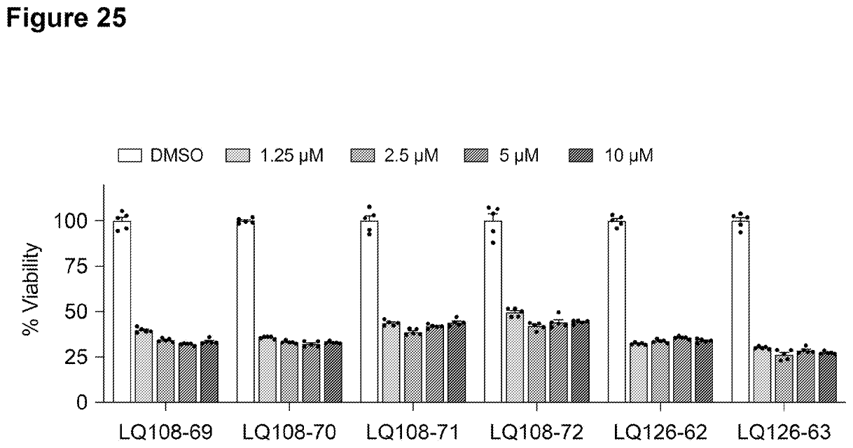

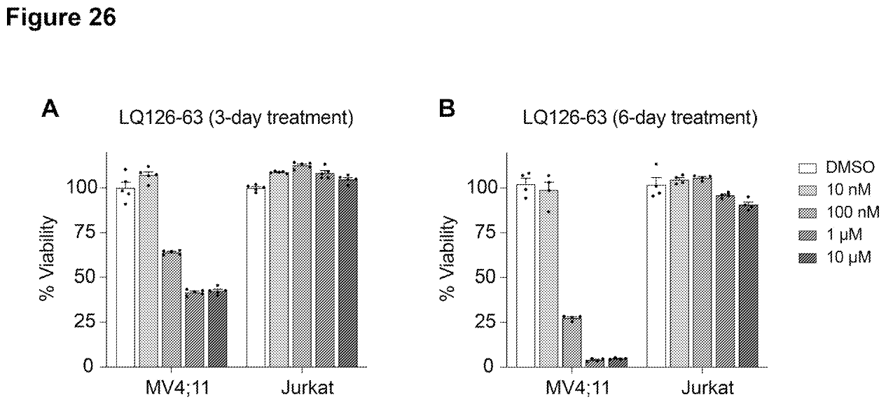

Furthermore, specific exemplary ENL degraders/disruptors were firstly characterized in ENL-dependent leukemia MV4; 11 cells stably expressing 3Flag-HA-tagged ENL to evaluate their ability in inducing degradation of ectopically expressed 3Flag-HA-ENL protein at 1 μM and 10 μM doses (FIG. 19A-D). Compounds achieved >50% ENL protein degradation at 10 μM were further characterized in the same cell line with 6 h treatment at 1 μM and 10 μM doses (FIG. 20A-B). A selected panel of compounds were tested by Western blotting for their efficiencies in reducing endogenous ENL protein levels in MV4; 11 cells at 1 μM and 10 μM with 6 h treatment (FIG. 21). Among them, compounds LQ108-69, LQ108-70, LQ108-71, LQ108-72, LQ126-62, and LQ126-63 were identified to be effective in reducing ENL protein levels in MV4; 11, MOLM13 and Jurkat cells in a concentration- and time-dependent manner (FIGS. 22 and 23). In addition, proteasome inhibitor MG132 can partially block the degradation of ENL protein induced by LQ108-63, LQ108-69, LQ108-70, LQ126-62 and LQ126-63 in MV4; 11 cells (FIG. 24), suggesting a MOA through proteasome-mediated protein degradation. Compounds LQ108-69, LQ108-70, LQ108-71, LQ108-72, LQ126-62, and LQ126-63 significantly suppressed MV4; 11 cell growth at low micromolar concentration (FIG. 25). Furthermore, degrader LQ126-63 strongly suppressed MV4; 11 cell growth at 100 nM dose but did not affect the growth of ENL-independent Jurkat cells (FIG. 26).

### Definition of Terms

As used herein, the terms “comprising” and “including” are used in their open, non-limiting sense.

“Alkyl” refers to a straight or branched hydrocarbon chain radical consisting solely of carbon and hydrogen atoms, containing no unsaturation. An alkyl may comprise one, two, three, four, five, six, seven, eight, nine, ten, eleven, twelve, thirteen, fourteen, fifteen, or sixteen carbon atoms. In certain embodiments, an alkyl comprises one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), pentyl, 3-methylhexyl, 2-methylhexyl, and the like.

“Alkenyl” refers to a straight or branched hydrocarbon chain radical group consisting solely of carbon and hydrogen atoms, containing at least one double bond. An alkenyl may comprise two, three, four, five, six, seven, eight, nine, ten, eleven, twelve, thirteen, fourteen, fifteen, or sixteen carbon atoms. In certain embodiments, an alkenyl comprises two to twelve carbon atoms (e.g., C₂-C₁₂ alkenyl). In certain embodiments, an alkenyl comprises two to eight carbon atoms (e.g., C₂-C₈ alkenyl). In certain embodiments, an alkenyl comprises two to six carbon atoms (e.g., C₂-C₆ alkenyl). In other embodiments, an alkenyl comprises two to four carbon atoms (e.g., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

The term “allyl,” as used herein, means a —CH₂CH═CH₂ group.

As used herein, the term “alkynyl” refers to a straight or branched hydrocarbon chain radical group consisting solely of carbon and hydrogen atoms, containing at least one triple bond. An alkynyl may comprise two, three, four, five, six, seven, eight, nine, ten, eleven, twelve, thirteen, fourteen, fifteen, or sixteen carbon atoms. In certain embodiments, an alkynyl comprises two to twelve carbon atoms (e.g., C₂-C₁₂ alkynyl). In certain embodiments, an alkynyl comprises two to eight carbon atoms (e.g., C₂-C₈ alkynyl). In other embodiments, an alkynyl has two to six carbon atoms (e.g., C₂-C₆ alkynyl). In other embodiments, an alkynyl has two to four carbon atoms (e.g., C₂-C₄ alkynyl). The alkynyl is attached to the rest of the molecule by a single bond. Examples of such groups include, but are not limited to, ethynyl, propynyl, 1-butynyl, 2-butynyl, 1-pentynyl, 2-pentynyl, 1-hexynyl, 2-hexynyl, 3-hexynyl, and the like.

The term “alkoxy”, as used herein, means an alkyl group as defined herein which is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

The term “aryl”, as used herein, “refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms. An aryl may comprise from six to eighteen carbon atoms, where at least one of the rings in the ring system is fully unsaturated, i.e., it contains a cyclic, delocalized (4n+2) π-electron system in accordance with the Hückel theory. In certain embodiments, an aryl comprises six to fourteen carbon atoms (C₆-C₁₄ aryl). In certain embodiments, an aryl comprises six to ten carbon atoms (C₆-C₁₀ aryl). Examples of such groups include, but are not limited to, phenyl, fluorenyl and naphthyl. The terms “Ph” and “phenyl,” as used herein, mean a —C₆H₅ group.

The term “heteroaryl”, refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s).

Examples of such groups include, but not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. In certain embodiments, an heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “heterocyclyl”, as used herein, means a non-aromatic, monocyclic, bicyclic, tricyclic, or tetracyclic radical having a total of from 4, 5, 6, 7, 8, 9, 10, 11, 12, or 13 atoms in its ring system, and containing from 3 to 12 carbon atoms and from 1 to 4 heteroatoms each independently selected from O, S and N, and with the proviso that the ring of said group does not contain two adjacent O atoms or two adjacent S atoms. A heterocyclyl group may include fused, bridged or spirocyclic ring systems. In certain embodiments, a heterocyclyl group comprises 3 to 10 ring atoms (3-10 membered heterocyclyl). In certain embodiments, a heterocyclyl group comprises 3 to 8 ring atoms (3-8 membered heterocyclyl). In certain embodiments, a heterocyclyl group comprises 4 to 8 ring atoms (4-8 membered heterocyclyl). In certain embodiments, a heterocyclyl group comprises 3 to 6 ring atoms (3-6 membered heterocyclyl). A heterocyclyl group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclyl group contains a sulfur atom, said sulfur atom may be oxidized with one or two oxygen atoms to afford either a sulfoxide or sulfone. An example of a 4 membered heterocyclyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclyl groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8,diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached).

The term “cycloalkyl” means a saturated, monocyclic, bicyclic, tricyclic, or tetracyclic radical having a total of from 4, 5, 6, 7, 8, 9, 10, 11, 12, or 13 carbon atoms in its ring system. A cycloalkyl may be fused, bridged or spirocyclic. In certain embodiments, a cycloalkyl comprises 3 to 8 carbon ring atoms (C₃-C₈ cycloalkyl). In certain embodiments, a cycloalkyl comprises 3 to 6 carbon ring atoms (C₃-C₆ cycloalkyl). Examples of such groups include, but are not limited to, cyclopropyl, cyclobutyl, cyclopentyl, cyclopentenyl, cyclohexyl, cycloheptyl, adamantyl, and the like.

The term “cycloalkylene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like.

The term “spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro[3.4]octane, and spiro[4.5]decane.

The term cyano” refers to a —C≡N group.

An “aldehyde” group refers to a —C(O)H group.

An “alkoxy” group refers to both an —O-alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂alkyl, as defined herein.

An “amino” group refers to an optionally substituted —NH₂.

An “aminoalkyl” group refers to an -alky-amino group, as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂aryl, as defined herein.

A “carbonyl” group refers to a —C(O)— group, as defined herein.

A “carboxylic acid” group refers to a —C(O)OH group.

A “cycloalkoxy” refers to a —O-cycloalkyl group, as defined herein.

A “halo” or “halogen” group refers to fluorine, chlorine, bromine or iodine.

A “haloalkyl” group refers to an alkyl group substituted with one or more halogen atoms.

A “hydroxy” group refers to an —OH group.

A “nitro” group refers to a —NO₂ group.

An “oxo” group refers to the ═O substituent.

A “trihalomethyl” group refers to a methyl substituted with three halogen atoms.

The term “substituted,” means that the specified group or moiety bears one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “optionally substituted” means that the specified group may be either unsubstituted or substituted by one or more substituents as defined herein. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present invention is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies.

“Pharmaceutically acceptable salt” includes both acid and base addition salts. A pharmaceutically acceptable salt of any one of the bivalent compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### Pharmaceutical Compositions

In some aspects, the compositions and methods described herein include the manufacture and use of pharmaceutical compositions and medicaments that include one or more bivalent compounds as disclosed herein. Also included are the pharmaceutical compositions themselves.

In some aspects, the compositions disclosed herein can include other compounds, drugs, or agents used for the treatment of cancer. For example, in some instances, pharmaceutical compositions disclosed herein can be combined with one or more (e.g., one, two, three, four, five, or less than ten) compounds. Such additional compounds can include, e.g., conventional chemotherapeutic agents known in the art. When co-administered, ENL degraders/disruptors disclosed herein can operate in conjunction with conventional chemotherapeutic agents to produce mechanistically additive or synergistic therapeutic effects.

In some aspects, the pH of the compositions disclosed herein can be adjusted with pharmaceutically acceptable acids, bases, or buffers to enhance the stability of the ENL degraders/disruptor or its delivery form.

Pharmaceutical compositions typically include a pharmaceutically acceptable carrier, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable carrier, adjuvant, or vehicle is a composition that can be administered to a patient, together with a compound of the invention, and which does not destroy the pharmacological activity thereof and is nontoxic when administered in doses sufficient to deliver a therapeutic amount of the compound. Exemplary conventional nontoxic pharmaceutically acceptable carriers, adjuvants, and vehicles include saline, solvents, dispersion media, coatings, antibacterial and antifungal agents, isotonic and absorption delaying agents, and the like, compatible with pharmaceutical administration.

In particular, pharmaceutically acceptable carriers, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

As used herein, the ENL degraders/disruptors disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The ENL degraders/disruptors disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivative thereof.

In particular, pharmaceutically acceptable salts of the ENL degraders/disruptors disclosed herein include, e.g., those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate, trifluoromethylsulfonate, and undecanoate. Salts derived from appropriate bases include, e.g., ENL alkali metal (e.g., sodium), ENL alkaline earth metal (e.g., magnesium), ammonium and N-(ENLyl)4+ salts. The invention also envisions the quaternization of any basic nitrogen-containing groups of the ENL degraders/disruptors disclosed herein. Water or oil-soluble or dispersible products can be obtained by such quaternization.

In some aspects, the pharmaceutical compositions disclosed herein can include an effective amount of one or more ENL degraders/disruptors. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cell growth, cell proliferation, or cancer).

In some aspects, pharmaceutical compositions can further include one or more additional compounds, drugs, or agents used for the treatment of cancer (e.g., conventional chemotherapeutic agents) in amounts effective for causing an intended effect or physiological outcome (e.g., treatment or prevention of cell growth, cell proliferation, or cancer).

In some aspects, the pharmaceutical compositions disclosed herein can be formulated for sale in the United States, import into the United States, or export from the United States.

### Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, the pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

For example, pharmaceutical compositions of this invention can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

For example, the pharmaceutical compositions of this invention can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

For example, the pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this invention can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of a compound of the formulae described herein (e.g., a ENL degraders/disruptors) and one or more additional compounds (e.g., one or more additional compounds, drugs, or agents used for the treatment of cancer or any other condition or disease, including conditions or diseases known to be associated with or caused by cancer), both the compound and the additional compound should be present at dosage levels of between about 1 to 100%, and more preferably between about 5 to 95% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

In some aspects, the pharmaceutical compositions disclosed herein can be included in a container, pack, or dispenser together with instructions for administration.

### Methods of Treatment

The methods disclosed herein contemplate administration of an effective amount of a compound or composition to achieve the desired or stated effect. Typically, the compounds or compositions of the invention will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some aspects, the present disclosure provides methods for using a composition comprising an ENL degrader/disruptor, including pharmaceutical compositions (indicated below as ‘X’) disclosed herein in the following methods:

Substance X for use as a medicament in the treatment of one or more diseases or conditions disclosed herein (e.g., cancer, referred to in the following examples as ‘Y’). Use of substance X for the manufacture of a medicament for the treatment of Y; and substance X for use in the treatment of Y.

In some aspects, the methods disclosed include the administration of a therapeutically effective amount of one or more of the compounds or compositions described herein to a subject (e.g., a mammalian subject, e.g., a human subject) who is in need, or who has been determined to be in need of, such treatment. In some aspects, the methods disclosed include selecting a subject and administering to the subject an effective amount of one or more of the compounds or compositions described herein, and optionally repeating administration as required for the prevention or treatment of cancer.

In some aspects, subject selection can include obtaining a sample from a subject (e.g., a candidate subject) and testing the sample for an indication that the subject is suitable for selection.

In some aspects, the subject can be confirmed or identified, e.g. by a health care professional, as having had or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition from which the subject is suffering (e.g., an ENL-mediated cancer). In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the compositions and methods of the present invention. In some aspects, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; a decrease in the rate of growth of tumor cells; a decrease in the rate of local or distant tumor metastasis; or reductions in one or more symptoms associated with one or more tumors in a subject relative to the subject's symptoms prior to treatment.

As used herein, the term “treating cancer” means causing a partial or complete decrease in the rate of growth of a tumor, and/or in the size of the tumor and/or in the rate of local or distant tumor metastasis, and/or the overall tumor burden in a subject, and/or any decrease in tumor survival, in the presence of a degrader/disruptor (e.g., an ENL degrader/disruptor) described herein.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention. Exemplary ENL-mediated diseases that can be treated with ENL degraders/disruptors include acute leukemia, mixed lineage leukemia (MLL)-rearranged leukemias, Wilms' tumor and other diseases that are dependent on ENL.

As used herein, the term “preventing a disease” (e.g., preventing cancer) in a subject means for example, to stop the development of one or more symptoms of a disease in a subject before they occur or are detectable, e.g., by the patient or the patient's doctor. Preferably, the disease (e.g., cancer) does not develop at all, i.e., no symptoms of the disease are detectable. However, it can also mean delaying or slowing of the development of one or more symptoms of the disease. Alternatively, or in addition, it can mean decreasing the severity of one or more subsequently developed symptoms.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

The ENL degraders/disruptors disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated and fluoro derivatives thereof.

### EXAMPLES

The following Examples describe the synthesis of exemplary ENL degrader/disrupter compounds according to the present invention.

### EXAMPLES

### Example 1

### Synthesis of Intermediate 4

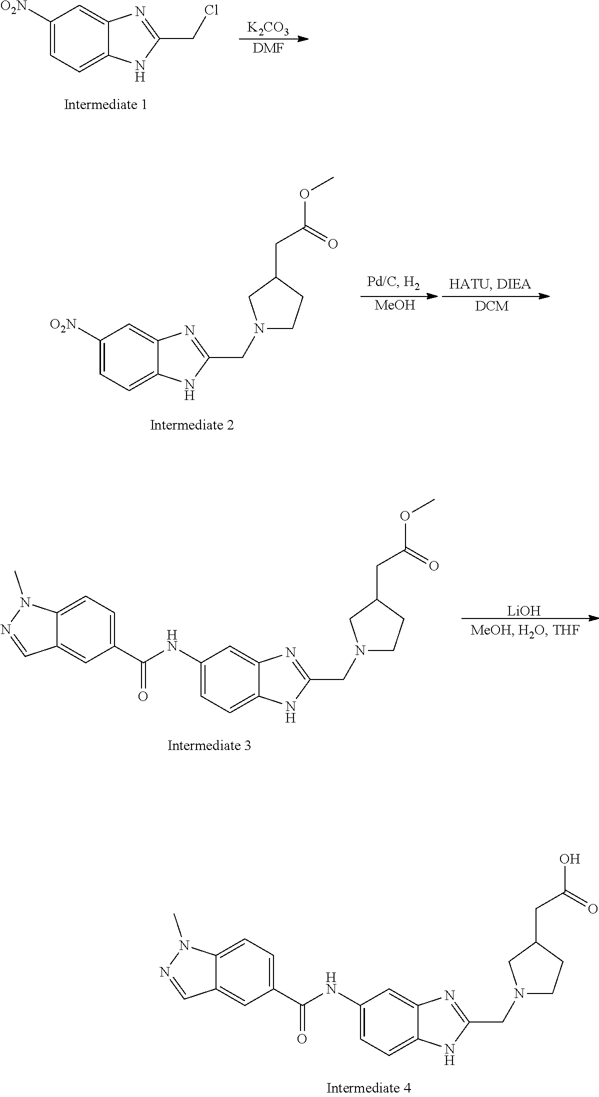

### Intermediate 2: Methyl 2-(1-((5-nitro-1H-benzo[d]imidazol-2-yl)methyl)pyrrolidin-3-yl)acetate

A solution of intermediate 1 (Moustakim et al., 2018b) (211 mg, 1 mmol) and Methyl 3-pyrrolidinylacetate hydrochloride (198 mg, 1.1 mmol) in 5 mL of DMF was treated with K₂CO₃ (276 mg, 2 mmol). The resulting mixture was stirred overnight at RT. After the reaction was completed, the reaction mixture was poured into ice water, aqueous phase was extracted with ethyl acetate. The combined organic phase was washed with brine twice, dried and concentrated. The resulting residue was purified by silica gel flash chromatography to give the compound as yellow oil (222 mg, 70%). ¹H NMR (600 MHz, Methanol-d₄) δ 8.58 (d, J=2.2 Hz, 1H), 8.24 (dd, J=8.9, 2.2 Hz, 1H), 7.77 (d, J=9.0 Hz, 1H), 4.84 (d, J=1.3 Hz, 2H), 3.98-3.86 (m, 1H), 3.77-3.62 (m, 5H), 3.43-3.34 (m, 1H), 2.94-2.84 (m, 1H), 2.71-2.60 (m, 2H), 2.46-2.37 (m, 1H), 1.94-1.84 (m, 1H). MS (ESI): m/z 319.2 [M+H]⁺.

### Intermediate 3: Methyl 2-(1-((5-(1-methyl-1H-indazole-5-carboxamido)-1H-benzo[d]imidazol-2-yl)methyl)pyrrolidin-3-yl)acetate

10% Pd on carbon (20 mg) was added to a solution of intermediate 2 (220 mg, 0.69 mmol) in MeOH, and the mixture was stirred under H₂ atmosphere overnight. The catalyst was removed by filtration through a pad of celite, the solvent was removed in vacuo and the residue was used in next step without further purification. The obtained intermediate was dissolved in dichloromethane and treated with 1-Methyl-1H-indazole-5-carboxylic acid (121 mg, 0.69 mmol), HATU (293 mg, 0.76 mmol) and DIEA (155 μL, 1.1 mmol). After being stirring 1 h at room temperature, the reaction mixture was washed with brine, dried and concentrated. The resulting residue was purified by silica gel flash chromatography to give the compound as yellow solid (223 mg, 72% for two steps). ¹H NMR (600 MHz, Methanol-d₄) δ 8.48 (s, 1H), 8.30 (d, J=2.0 Hz, 1H), 8.19 (s, 1H), 8.06 (dd, J=8.8, 1.7 Hz, 1H), 7.72-7.66 (m, 2H), 7.57 (dd, J=8.7, 2.0 Hz, 1H), 4.70 (s, 2H), 4.15 (s, 3H), 3.85-3.79 (m, 1H), 3.71 (s, 3H), 3.64-3.54 (m, 2H), 3.25 (t, J=10.3 Hz, 1H), 2.90-2.82 (m, 1H), 2.69-2.58 (m, 2H), 2.43-2.34 (m, 1H), 1.89-1.80 (m, 1H). MS (ESI): m/z 447.3 [M+H]⁺.

### Intermediate 4: 2-(1-((5-(1-methyl-1H-indazole-5-carboxamido)-1H-benzo[d]imidazol-2-yl)methyl)pyrrolidin-3-yl)acetic acid

To a solution of intermediate 3 (300 mg, 0.67 mmol) in 5 mL MeOH, 5 mL H₂O, and 5 mL THF, LiOH (30 mg, 1 mmol) was added. The mixture was stirred at RT overnight. Then the mixture was purified by reverse phase C18 column (10%-100% methanol/0.1% TFA in water) to afford intermediate 4 as white solid in TFA salt form (486 mg, 89%). ¹H NMR (600 MHz, Methanol-d₄) δ 8.46 (dd, J=1.7, 0.8 Hz, 1H), 8.31 (d, J=1.9 Hz, 1H), 8.17 (d, J=0.9 Hz, 1H), 8.05 (dd, J=8.8, 1.7 Hz, 1H), 7.71-7.66 (m, 2H), 7.60 (dd, J=8.8, 2.0 Hz, 1H), 4.76 (s, 2H), 4.13 (s, 3H), 3.83 (dd, J=11.5, 8.1 Hz, 1H), 3.66-3.55 (m, 2H), 3.29 (dd, J=11.5, 8.8 Hz, 1H), 2.90-2.81 (m, 1H), 2.65-2.55 (m, 2H), 2.43-2.36 (m, 1H), 1.91-1.82 (m, 1H). MS (ESI): m/z 433.4 [M+H]⁺.

### Example 2

### Synthesis of LQ076-46 (Actual Name of Compounds First!)

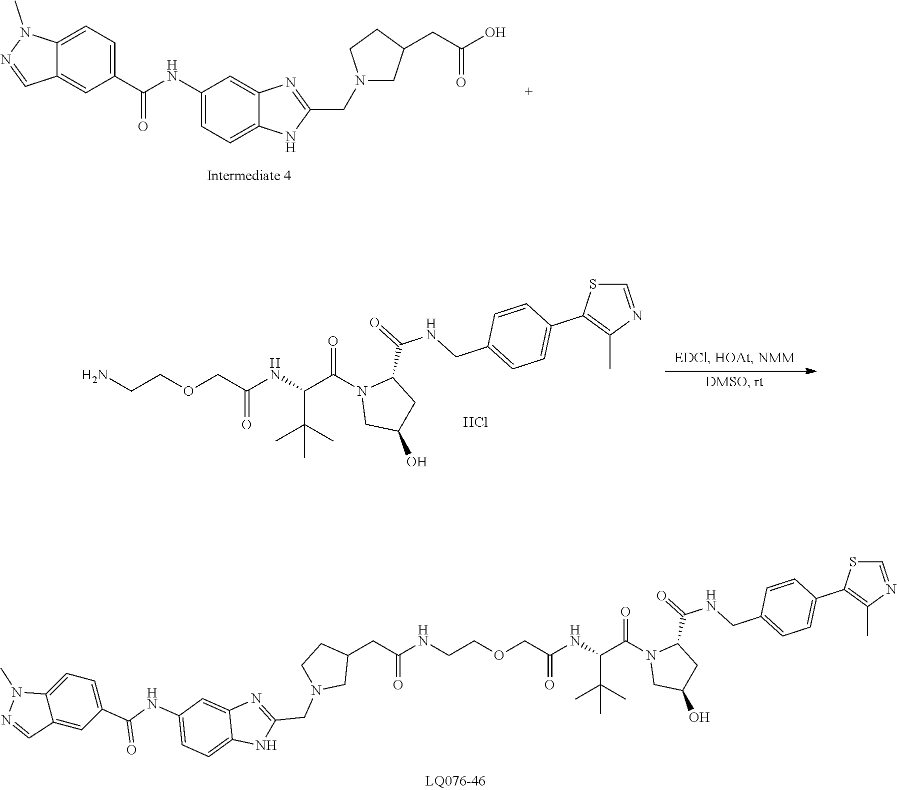 [SMILES: Cn1ncc2cc(C(=O)Nc3ccc4[nH]c(CN5CCC(CC(=O)O)C5)nc4c3)ccc21 ; Cc1ncsc1-c1ccc(CNC(=O)[C@@H]2C[C@@H](O)CN2C(=O)[C@@H](NC(=O)COCCN)C(C)(C)C)cc1 ; Cl ; Cc1ncsc1-c1ccc(CNC(=O)[C@@H]2C[C@@H](O)CN2C(=O)[C@@H](NC(=O)COCCNC(=O)CC2CCN(Cc3nc4cc(NC(=O)c5ccc6c(cnn6C)c5)ccc4[nH]3)C2)C(C)(C)C)cc1]

To a solution of Intermediate 4 (12 mg, 0.02 mmol) in DMSO (1 mL) were added (2S,4R)-1-((S)-2-(2-(2-aminoethoxy)acetamido)-3,3-dimethylbutanoyl)-4-hydroxy-N-(4-(4-methylthiazol-5-yl)benzyl)pyrrolidine-2-carboxamide (11.4 mg, 0.02 mmol, 1.0 equiv), EDCI (1-ethyl-3-(3-dimethylaminopropyl)carbodiimide) (5.8 mg, 0.03 mmol, 1.5 equiv), HOAt (1-hydroxy-7-azabenzo-triazole) (4.1 mg, 0.03 mmol, 1.5 equiv), and NMM (N-Methylmorpholine) (6.1 mg, 0.06 mmol, 3.0 equiv). After being stirred overnight at room temperature, the resulting mixture was purified by preparative HPLC (5%-60% acetonitrile/0.1% TFA in H₂O) to afford LQ076-46 as white solid in TFA salt form (19.3 mg, 81%). ¹H NMR (600 MHz, Methanol-d₄) δ 9.05 (s, 1H), 8.44 (s, 1H), 8.30-8.24 (m, 1H), 8.03 (dd, J=8.9, 1.6 Hz, 1H), 7.68 (d, J=8.8 Hz, 1H), 7.66-7.61 (m, 1H), 7.53-7.50 (m, 1H), 7.46-7.37 (m, 4H), 4.73-4.64 (m, 2H), 4.60-4.49 (m, 4H), 4.41-4.30 (m, 1H), 4.13 (s, 3H), 4.07-3.95 (m, 1H), 3.95-3.87 (m, 1H), 3.84-3.77 (m, 1H), 3.77-3.68 (m, 1H), 3.67-3.50 (m, 3H), 3.29-3.22 (m, 1H), 2.87-2.77 (m, 1H), 2.61-2.50 (m, 3H), 2.45 (d, J=7.8 Hz, 3H), 2.43-2.40 (m, 1H), 2.39-2.20 (m, 3H), 2.14-2.06 (m, 2H), 1.91-1.78 (m, 1H), 1.04 (s, 9H). HRMS m/z [M+H]⁺ calcd for C₄₉H₆₀N₁₁O₇S⁺ 946.4392, found 946.4385.

### Example 3

### Synthesis of LQ076-47

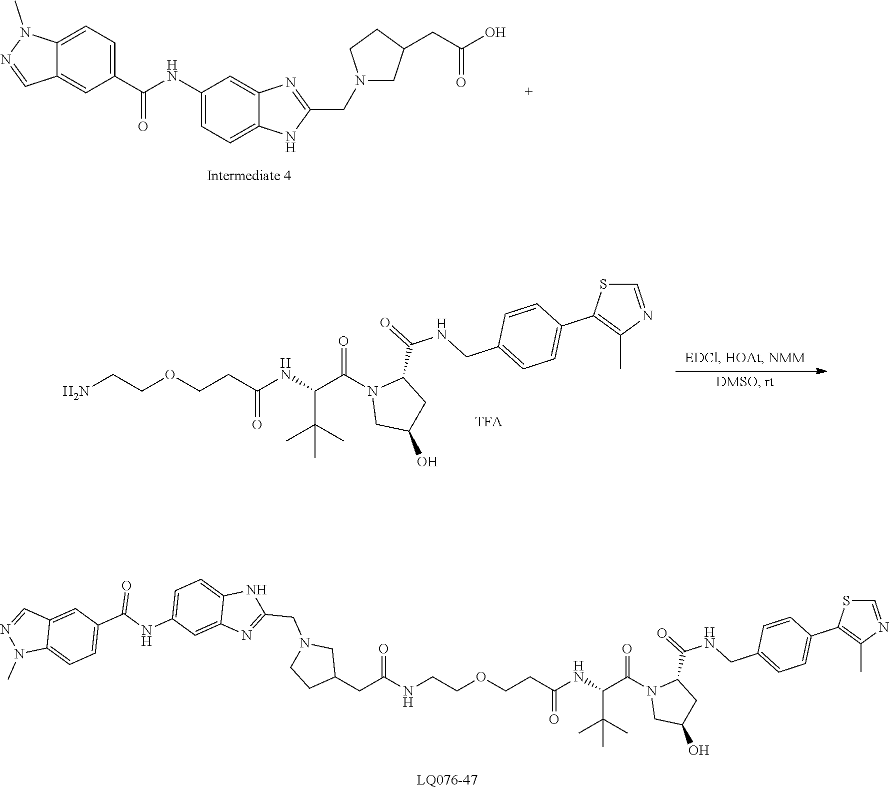 [SMILES: Cn1ncc2cc(C(=O)Nc3ccc4[nH]c(CN5CCC(CC(=O)O)C5)nc4c3)ccc21 ; Cc1ncsc1-c1ccc(CNC(=O)[C@@H]2C[C@@H](O)CN2C(=O)[C@@H](NC(=O)CCOCCN)C(C)(C)C)cc1 ; Cc1ncsc1-c1ccc(CNC(=O)[C@@H]2C[C@@H](O)CN2C(=O)[C@@H](NC(=O)CCOCCNC(=O)CC2CCN(Cc3nc4cc(NC(=O)c5ccc6c(cnn6C)c5)ccc4[nH]3)C2)C(C)(C)C)cc1 ; "TFA"]

LQ076-47 was synthesized following the standard procedure for preparing LQ076-46 from intermediate 4 (12 mg, 0.02 mmol), (2S,4R)-1-((S)-2-(3-(2-aminoethoxy)propanamido)-3,3-dimethylbutanoyl)-4-hydroxy-N-(4-(4-methylthiazol-5-yl)benzyl)pyrrolidine-2-carboxamide (15.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-47 was obtained as white solid in TFA salt form (20.2 mg, 85%). ¹H NMR (600 MHz, Methanol-d₄) δ 9.05 (s, 1H), 8.47-8.45 (m, 1H), 8.34-8.33 (m, 1H), 8.19-8.17 (m, 1H), 8.05 (dd, J=8.8, 1.7 Hz, 1H), 7.69 (dd, J=8.8, 2.0 Hz, 2H), 7.61-7.59 (m, 1H), 7.47-7.40 (m, 4H), 4.77 (s, 2H), 4.68-4.66 (m, 1H), 4.63-4.57 (m, 1H), 4.53-4.47 (m, 2H), 4.42-4.38 (m, 1H), 4.14 (s, 3H), 3.91 (d, J=11.0 Hz, 1H), 3.82 (dd, J=11.0, 3.8 Hz, 1H), 3.78-3.69 (m, 3H), 3.65-3.51 (m, 4H), 3.31-3.26 (m, 1H), 2.86-2.78 (m, 1H), 2.57-2.41 (m, 11H), 2.38-2.30 (m, 1H), 2.28-2.23 (m, 1H), 2.12-2.06 (m, 1H), 1.89-1.81 (m, 1H), 1.05 (s, 9H). HRMS m/z [M+H]⁺ calcd for C₅₀H₆₂N₁₁O₇S⁺ 960.4549, found 960.4576.

### Example 4

### Synthesis of LQ076-48

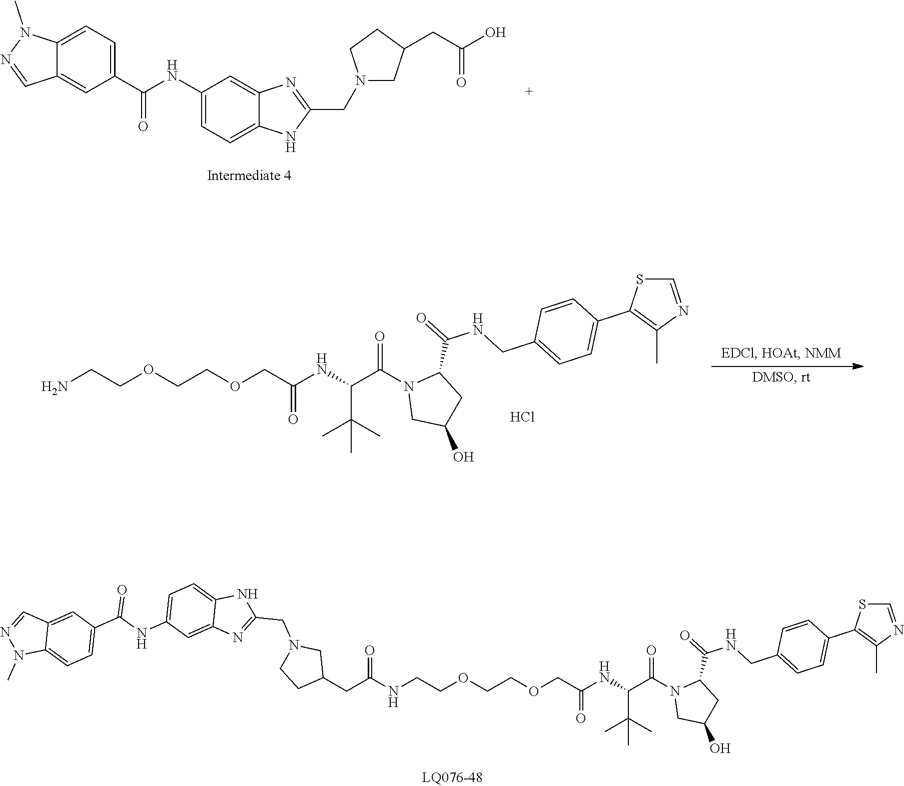 [SMILES: Cn1ncc2cc(C(=O)Nc3ccc4[nH]c(CN5CCC(CC(=O)O)C5)nc4c3)ccc21 ; Cc1ncsc1-c1ccc(CNC(=O)[C@@H]2C[C@@H](O)CN2C(=O)[C@@H](NC(=O)COCCOCCN)C(C)(C)C)cc1 ; Cl ; Cc1ncsc1-c1ccc(CNC(=O)[C@@H]2C[C@@H](O)CN2C(=O)[C@@H](NC(=O)COCCOCCNC(=O)CC2CCN(Cc3nc4cc(NC(=O)c5ccc6c(cnn6C)c5)ccc4[nH]3)C2)C(C)(C)C)cc1]

LQ076-48 was synthesized following the standard procedure for preparing LQ076-46 from intermediate 4 (12 mg, 0.02 mmol), (2S,4R)-1-((S)-2-(2-(2-(2-aminoethoxy)ethoxy)acetamido)-3,3-dimethylbutanoyl)-4-hydroxy-N-(4-(4-methylthiazol-5-yl)benzyl)pyrrolidine-2-carboxamide (13 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-48 was obtained as white solid in TFA salt form (21.1 mg, 88%). ¹H NMR (600 MHz, Methanol-d₄) δ 9.08 (s, 1H), 8.46 (s, 1H), 8.37-8.33 (m, 1H), 8.18 (s, 1H), 8.04 (dd, J=8.8, 1.7 Hz, 1H), 7.70-7.66 (m, 2H), 7.62-7.59 (m, 1H), 7.47-7.37 (m, 4H), 4.81-4.74 (m, 3H), 4.64-4.56 (m, 1H), 4.54-4.47 (m, 2H), 4.39 (d, J=15.3 Hz, 1H), 4.13 (s, 3H), 4.05-3.97 (m, 2H), 3.91-3.80 (m, 2H), 3.76-3.48 (m, 11H), 3.30-3.24 (m, 1H), 2.87-2.79 (m, 1H), 2.55-2.40 (m, 5H), 2.35-2.25 (m, 2H), 2.12-2.06 (m, 1H), 1.90-1.79 (m, 1H), 1.06 (s, 9H). HRMS m/z [M+H]⁺ calcd for C₅₁H₆₄N₁₁O₈S⁺ 990.4655, found 990.4740.

### Example 5

### Synthesis of LQ076-49

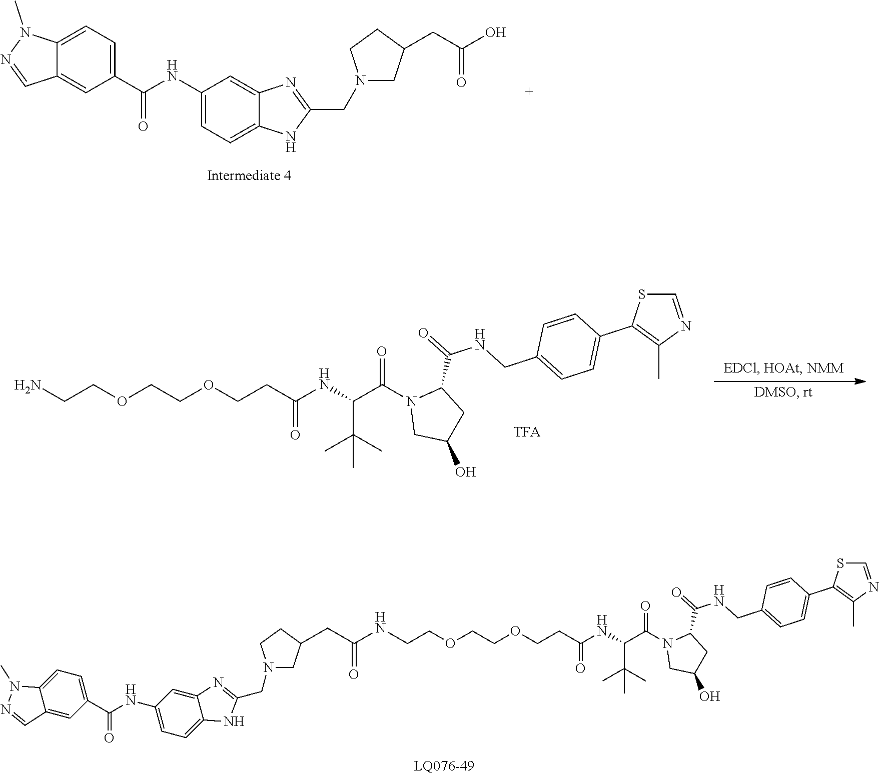 [SMILES: Cn1ncc2cc(C(=O)Nc3ccc4[nH]c(CN5CCC(CC(=O)O)C5)nc4c3)ccc21 ; Cc1ncsc1-c1ccc(CNC(=O)[C@@H]2C[C@@H](O)CN2C(=O)[C@@H](NC(=O)CCOCCOCCN)C(C)(C)C)cc1 ; Cc1ncsc1-c1ccc(CNC(=O)[C@@H]2C[C@@H](O)CN2C(=O)[C@@H](NC(=O)CCOCCOCCNC(=O)CC2CCN(Cc3nc4cc(NC(=O)c5ccc6c(cnn6C)c5)ccc4[nH]3)C2)C(C)(C)C)cc1 ; "TFA"]

LQ076-49 was synthesized following the standard procedure for preparing LQ076-46 from intermediate 4 (12 mg, 0.02 mmol), (2S,4R)-1-((S)-2-(3-(2-(2-aminoethoxy)ethoxy)propanamido)-3,3-dimethylbutanoyl)-4-hydroxy-N-(4-(4-methylthiazol-5-yl)benzyl)pyrrolidine-2-carboxamide (16.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-49 was obtained as white solid in TFA salt form (17.2 mg, 70%). ¹H NMR (600 MHz, Methanol-d₄) δ 9.06 (s, 1H), 8.46 (d, J=1.4 Hz, 1H), 8.34 (s, 1H), 8.18 (s, 1H), 8.04 (dd, J=8.8, 1.7 Hz, 1H), 7.71-7.67 (m, 2H), 7.61 (dd, J=8.8, 2.0 Hz, 1H), 7.48-7.40 (m, 4H), 4.78 (s, 2H), 4.67-4.65 (m, 1H), 4.60-4.56 (m, 1H), 4.54-4.49 (m, 2H), 4.40-4.35 (m, 1H), 4.13 (s, 3H), 3.90 (d, J=11.0 Hz, 1H), 3.83-3.70 (m, 4H), 3.67-3.51 (m, 9H), 3.38-3.34 (m, 2H), 3.31-3.27 (m, 1H), 2.87-2.80 (m, 1H), 2.60-2.55 (m, 1H), 2.52-2.40 (m, 6H), 2.38-2.32 (m, 1H), 2.27-2.21 (m, 1H), 2.12-2.05 (m, 1H), 1.90-1.82 (m, 1H), 1.05 (s, 9H). HRMS m/z [M+H]⁺ calcd for C₅₂H₆₆N₁₁O₈S⁺ 1004.4811, found 1004.4790.

### Example 6

### Synthesis of LQ076-50

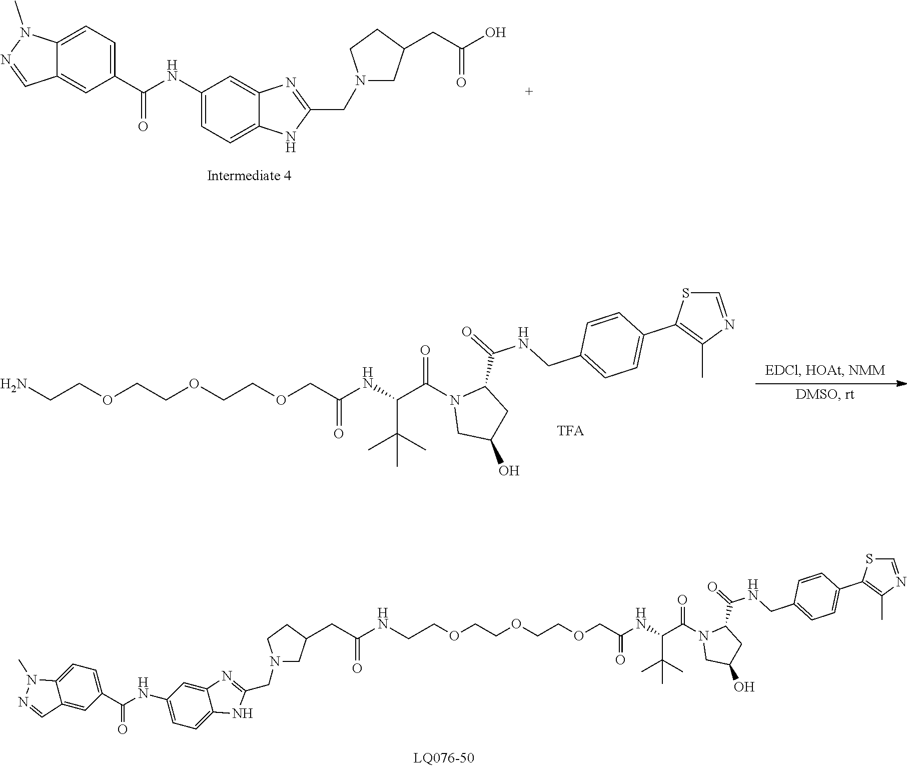 [SMILES: Cn1ncc2cc(C(=O)Nc3ccc4[nH]c(CN5CCC(CC(=O)O)C5)nc4c3)ccc21 ; Cc1ncsc1-c1ccc(CNC(=O)[C@@H]2C[C@@H](O)CN2C(=O)[C@@H](NC(=O)COCCOCCOCCN)C(C)(C)C)cc1 ; Cc1ncsc1-c1ccc(CNC(=O)[C@@H]2C[C@@H](O)CN2C(=O)[C@@H](NC(=O)COCCOCCOCCNC(=O)CC2CCN(Cc3nc4cc(NC(=O)c5ccc6c(cnn6C)c5)ccc4[nH]3)C2)C(C)(C)C)cc1 ; "TFA"]

LQ076-50 was synthesized following the standard procedure for preparing LQ076-46 from intermediate 4 (12 mg, 0.02 mmol), (2S,4R)-1-((S)-14-amino-2-(tert-butyl)-4-oxo-6,9,12-trioxa-3-azatetradecanoyl)-4-hydroxy-N-(4-(4-methylthiazol-5-yl)benzyl)pyrrolidine-2-carboxamide (17.1 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-50 was obtained as white solid in TFA salt form (18.9 mg, 75%). ¹H NMR (600 MHz, Methanol-d₄) δ 9.07 (s, 1H), 8.46 (d, J=1.5 Hz, 1H), 8.36 (d, J=2.0 Hz, 1H), 8.17 (d, J=0.9 Hz, 1H), 8.04 (dd, J=8.8, 1.7 Hz, 1H), 7.71-7.66 (m, 2H), 7.63-7.61 (m, 1H), 7.48-7.41 (m, 4H), 4.80 (s, 2H), 4.68 (d, J=4.4 Hz, 1H), 4.61-4.57 (m, 1H), 4.55-4.49 (m, 2H), 4.40-4.34 (m, 1H), 4.13 (s, 3H), 4.08-4.04 (m, 2H), 3.90-3.86 (m, 1H), 3.82-3.75 (m, 2H), 3.72-3.54 (m, 11H), 3.51 (t, J=5.4 Hz, 2H), 3.37-3.34 (m, 2H), 2.87-2.81 (m, 1H), 2.51-2.46 (m, 4H), 2.42 (dd, J=14.9, 8.0 Hz, 1H), 2.38-2.31 (m, 1H), 2.27-2.22 (m, 1H), 2.12-2.06 (m, 1H), 1.89-1.82 (m, 1H), 1.06 (s, 9H). HRMS m/z [M+H]⁺ calcd for C₅₃H₆₈N₁₁O₉S⁺ 1034.4917, found 1034.4932.

### Example 7

### Synthesis of LQ076-51

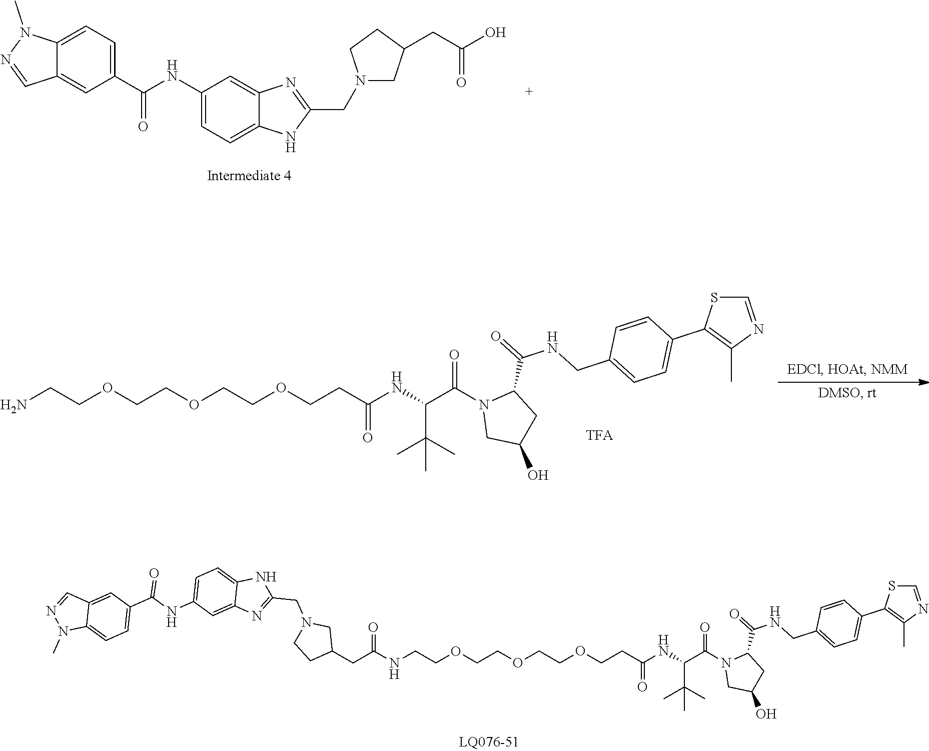 [SMILES: Cn1ncc2cc(C(=O)Nc3ccc4[nH]c(CN5CCC(CC(=O)O)C5)nc4c3)ccc21 ; Cc1ncsc1-c1ccc(CNC(=O)[C@@H]2C[C@@H](O)CN2C(=O)[C@@H](NC(=O)CCOCCOCCOCCN)C(C)(C)C)cc1 ; Cc1ncsc1-c1ccc(CNC(=O)[C@@H]2C[C@@H](O)CN2C(=O)[C@@H](NC(=O)CCOCCOCCOCCNC(=O)CC2CCN(Cc3nc4cc(NC(=O)c5ccc6c(cnn6C)c5)ccc4[nH]3)C2)C(C)(C)C)cc1 ; "TFA"]

LQ076-51 was synthesized following the standard procedure for preparing LQ076-46 from intermediate 4 (12 mg, 0.02 mmol), (2S,4R)-1-((S)-1-amino-14-(tert-butyl)-12-oxo-3,6,9-trioxa-13-azapentadecan-15-oyl)-4-hydroxy-N-(4-(4-methylthiazol-5-yl)benzyl)pyrrolidine-2-carboxamide (17.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-51 was obtained as white solid in TFA salt form (18.3 mg, 72%). ¹H NMR (600 MHz, Methanol-d₄) δ 9.11 (s, 1H), 8.48-8.45 (m, 1H), 8.36 (d, J=2.0 Hz, 1H), 8.18 (d, J=0.9 Hz, 1H), 8.05 (dd, J=8.8, 1.7 Hz, 1H), 7.73-7.66 (m, 2H), 7.63 (dd, J=8.8, 2.0 Hz, 1H), 7.49-7.41 (m, 4H), 4.80 (s, 2H), 4.66 (d, J=2.8 Hz, 1H), 4.62-4.56 (m, 1H), 4.55-4.49 (m, 2H), 4.40-4.35 (m, 1H), 4.13 (s, 3H), 3.90 (d, J=11.0 Hz, 1H), 3.83-3.69 (m, 4H), 3.67-3.56 (m, 10H), 3.53 (t, J=5.4 Hz, 2H), 3.39-3.35 (m, 2H), 3.31-3.29 (m, OH), 2.88-2.81 (m, 1H), 2.62-2.55 (m, 1H), 2.52-2.41 (m, 6H), 2.39-2.32 (m, 1H), 2.27-2.22 (m, 1H), 2.12-2.06 (m, 1H), 1.90-1.82 (m, 1H), 1.05 (s, 9H). HRMS m/z [M+H]⁺ calcd for C₅₄H₇₀N₁₁O₉S⁺ 1048.5073, found 1048.5066.

### Example 8

### Synthesis of LQ076-52

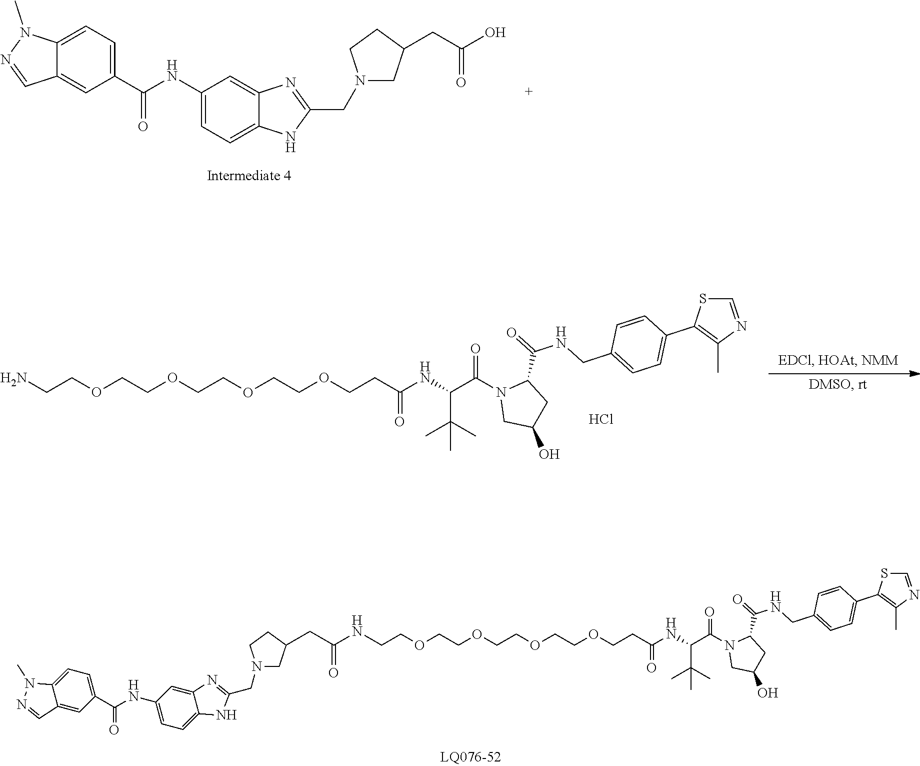 [SMILES: Cn1ncc2cc(C(=O)Nc3ccc4[nH]c(CN5CCC(CC(=O)O)C5)nc4c3)ccc21 ; Cc1ncsc1-c1ccc(CNC(=O)[C@@H]2C[C@@H](O)CN2C(=O)[C@@H](NC(=O)CCOCCOCCOCCOCCN)C(C)(C)C)cc1 ; Cl ; Cc1ncsc1-c1ccc(CNC(=O)[C@@H]2C[C@@H](O)CN2C(=O)[C@@H](NC(=O)CCOCCOCCOCCOCCNC(=O)CC2CCN(Cc3nc4cc(NC(=O)c5ccc6c(cnn6C)c5)ccc4[nH]3)C2)C(C)(C)C)cc1]

LQ076-52 was synthesized following the standard procedure for preparing LQ076-46 from intermediate 4 (12 mg, 0.02 mmol), (2S,4R)-1-((S)-1-amino-17-(tert-butyl)-15-oxo-3,6,9,12-tetraoxa-16-azaoctadecan-18-oyl)-4-hydroxy-N-(4-(4-methylthiazol-5-yl)benzyl)pyrrolidine-2-carboxamide (14.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-52 was obtained as white solid in TFA salt form (17.9 mg, 68%). ¹H NMR (600 MHz, Methanol-d₄) δ 9.09 (s, 1H), 8.48-8.46 (m, 1H), 8.35 (d, J=2.0 Hz, 1H), 8.18 (d, J=0.9 Hz, 1H), 8.05 (dd, J=8.8, 1.6 Hz, 1H), 7.71-7.67 (m, 2H), 7.62 (dd, J=8.8, 2.0 Hz, 1H), 7.49-7.41 (m, 4H), 4.80 (s, 2H), 4.67-4.64 (m, 1H), 4.61-4.48 (m, 3H), 4.40-4.35 (m, 1H), 4.13 (s, 3H), 3.90 (d, J=11.0 Hz, 1H), 3.83-3.69 (m, 4H), 3.67-3.57 (m, 14H), 3.53 (t, J=5.4 Hz, 2H), 3.39-3.35 (m, 2H), 3.32-3.28 (m, 1H), 2.88-2.81 (m, 1H), 2.61-2.55 (m, 1H), 2.53-2.41 (m, 6H), 2.39-2.32 (m, 1H), 2.26-2.21 (m, 1H), 2.11-2.06 (m, 1H), 1.90-1.83 (m, 1H), 1.05 (s, 9H). HRMS m/z [M+H]⁺ calcd for C₅₆H₇₄N₁₁O₁₀S⁺ 1092.5335, found 1092.5349.

### Example 9

### Synthesis of LQ076-53

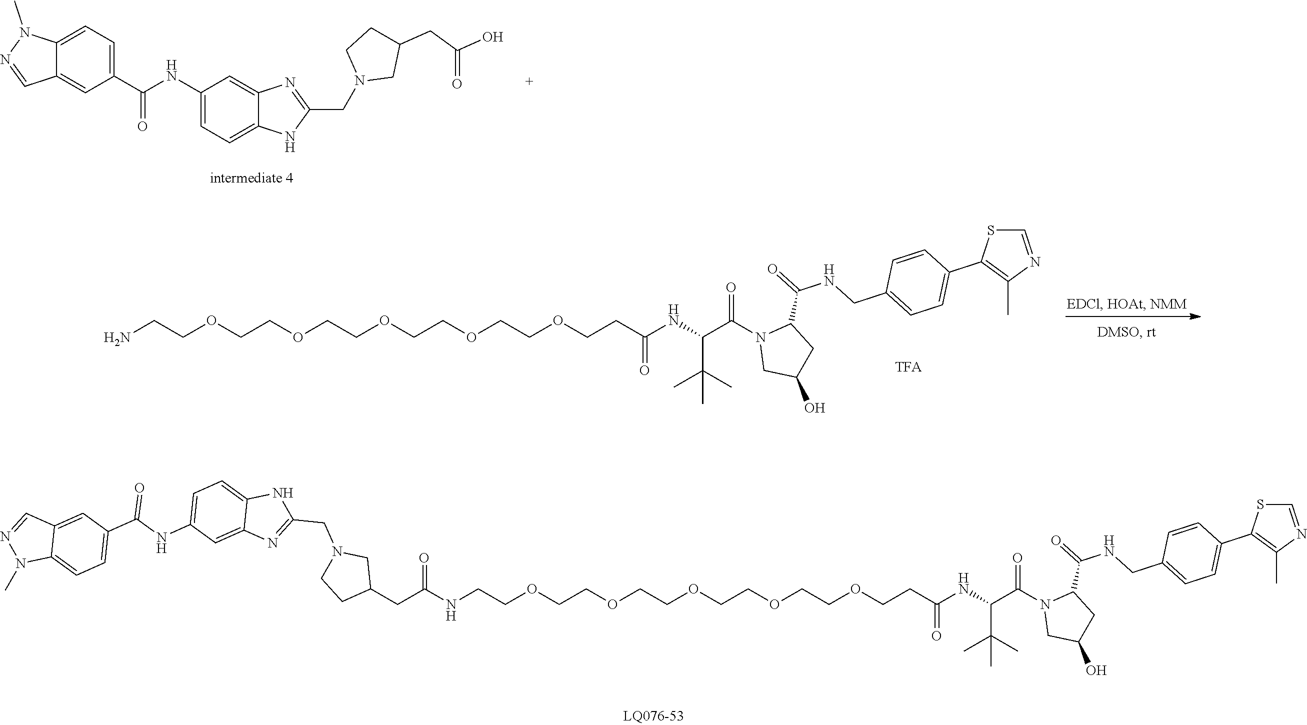

LQ076-53 was synthesized following the standard procedure for preparing LQ076-46 from intermediate 4 (12 mg, 0.02 mmol), (2S,4R)-1-((S)-1-amino-20-(tert-butyl)-18-oxo-3,6,9,12,15-pentaoxa-19-azahenicosan-21-oyl)-4-hydroxy-N-(4-(4-methylthiazol-5-yl)benzyl)pyrrolidine-2-carboxamide (18 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-53 was obtained as white solid in TFA salt form (17.7 mg, 65%). ¹H NMR (600 MHz, Methanol-d₄) δ 9.13 (s, 1H), 8.48-8.45 (m, 1H), 8.37 (d, J=2.0 Hz, 1H), 8.18 (s, 1H), 8.05 (dd, J=8.8, 1.7 Hz, 1H), 7.73-7.67 (m, 2H), 7.63 (dd, J=8.8, 1.9 Hz, 1H), 7.50-7.42 (m, 4H), 4.81 (s, 2H), 4.66-4.64 (m, 1H), 4.62-4.50 (m, 3H), 4.37 (d, J=15.5 Hz, 1H), 4.13 (s, 3H), 3.90 (d, J=11.0 Hz, 1H), 3.82-3.69 (m, 4H), 3.66-3.56 (m, 18H), 3.53 (t, J=5.4 Hz, 2H), 3.39-3.35 (m, 2H), 3.32-3.30 (m, 1H), 2.88-2.81 (m, 1H), 2.61-2.55 (m, 1H), 2.53-2.42 (m, 6H), 2.39-2.33 (m, 1H), 2.26-2.21 (m, 1H), 2.12-2.06 (m, 1H), 1.90-1.83 (m, 1H), 1.05 (s, 9H). HRMS m/z [M+H]⁺ calcd for C₅₈H₇₈N₁₁O₁₁S⁺ 1136.5597, found 1136.5645.

### Example 10

### Synthesis of LQ076-54

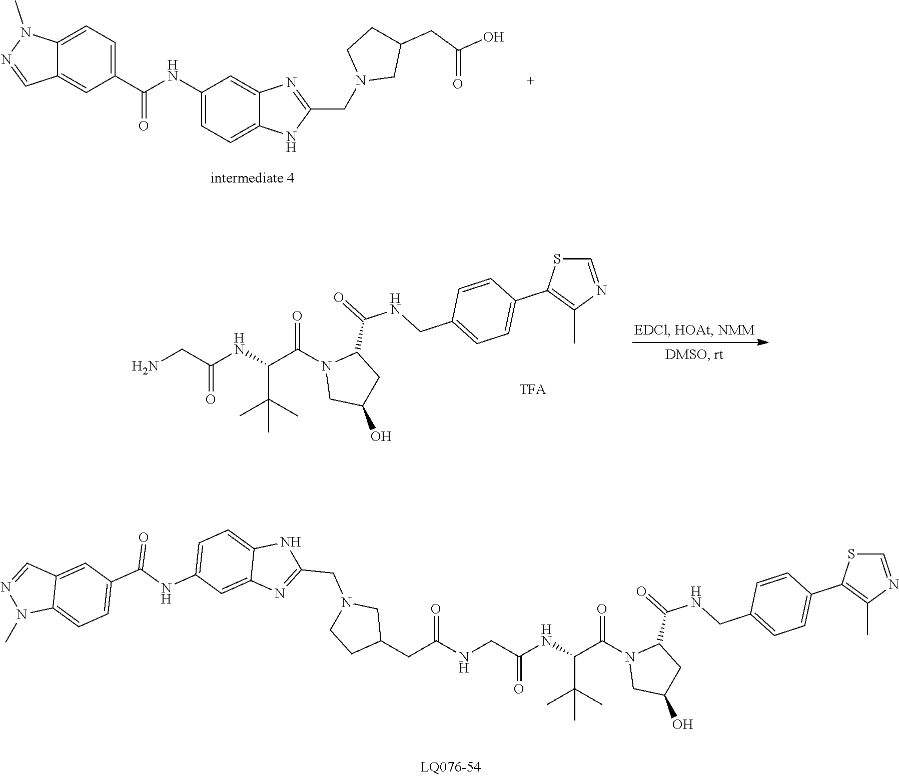

LQ076-54 was synthesized following the standard procedure for preparing LQ076-46 from intermediate 4 (12 mg, 0.02 mmol), (2S,4R)-1-((S)-2-(2-aminoacetamido)-3,3-dimethylbutanoyl)-4-hydroxy-N-(4-(4-methylthiazol-5-yl)benzyl)pyrrolidine-2-carboxamide (14.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-54 was obtained as white solid in TFA salt form (18.5 mg, 82%). ¹H NMR (600 MHz, Methanol-d₄) δ 9.08 (s, 1H), 8.47-8.44 (m, 1H), 8.36 (d, J=1.9 Hz, 1H), 8.18 (s, 1H), 8.06-8.02 (m, 1H), 7.71-7.67 (m, 2H), 7.63-7.60 (m, 1H), 7.46-7.39 (m, 4H), 4.79 (s, 2H), 4.61 (d, J=3.4 Hz, 1H), 4.59-4.55 (m, 1H), 4.52-4.46 (m, 2H), 4.37 (dd, J=15.5, 4.1 Hz, 1H), 4.13 (s, 3H), 3.94-3.83 (m, 3H), 3.81-3.75 (m, 2H), 3.69-3.62 (m, 1H), 3.60-3.54 (m, 1H), 3.40-3.35 (m, 1H), 2.91-2.84 (m, 1H), 2.61-2.57 (m, 1H), 2.52-2.46 (m, 4H), 2.41-2.35 (m, 1H), 2.26-2.21 (m, 1H), 2.11-2.05 (m, 1H), 1.97-1.89 (m, 1H), 1.04 (s, 9H). HRMS m/z [M+H]⁺ calcd for C₄₇H₅₆N₁₁O₆S⁺ 902.4130, found 902.4128.

### Example 11

### Synthesis of LQ076-55

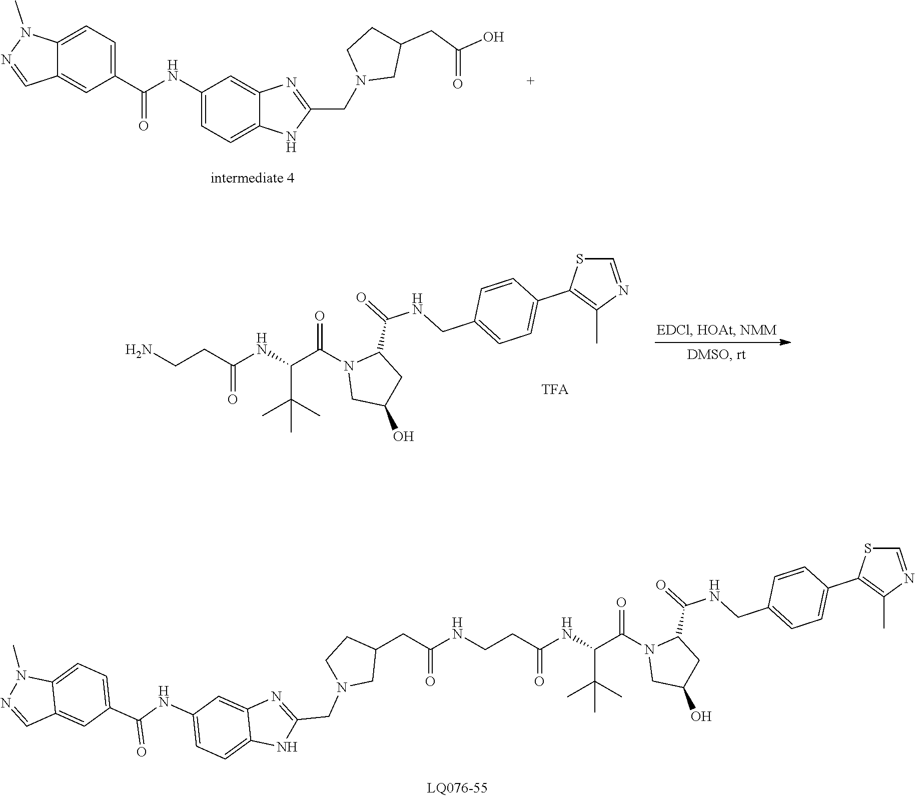

LQ076-55 was synthesized following the standard procedure for preparing LQ076-46 from intermediate 4 (12 mg, 0.02 mmol), (2S,4R)-1-((S)-2-(3-aminopropanamido)-3,3-dimethylbutanoyl)-4-hydroxy-N-(4-(4-methylthiazol-5-yl)benzyl)pyrrolidine-2-carboxamide (14.6 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-55 was obtained as white solid in TFA salt form (17.4 mg, 76%). ¹H NMR (600 MHz, Methanol-d₄) δ 9.04 (d, J=2.3 Hz, 1H), 8.46 (s, 1H), 8.34-8.31 (m, 1H), 8.18 (s, 1H), 8.04 (dd, J=8.8, 1.7 Hz, 1H), 7.70-7.66 (m, 2H), 7.61-7.58 (m, 1H), 7.48-7.36 (m, 4H), 4.82-4.75 (m, 2H), 4.62 (d, J=2.4 Hz, 1H), 4.59-4.55 (m, 1H), 4.53-4.50 (m, 1H), 4.49-4.46 (m, 1H), 4.41-4.36 (m, 1H), 4.14 (s, 3H), 3.98-3.94 (m, 1H), 3.83-3.75 (m, 2H), 3.67-3.61 (m, 1H), 3.60-3.54 (m, 1H), 3.53-3.46 (m, 1H), 3.43-3.37 (m, 1H), 3.31-3.27 (m, 1H), 2.85-2.78 (m, 1H), 2.56-2.44 (m, 6H), 2.43-2.31 (m, 2H), 2.30-2.24 (m, 1H), 2.13-2.07 (m, 1H), 1.89-1.81 (m, 1H), 1.04 (s, 9H). HRMS m/z [M+H]⁺ calcd for C₄₈H₅₈N₁₁O₆S⁺ 916.4287, found 916.4319.

### Example 12

### Synthesis of LQ076-56

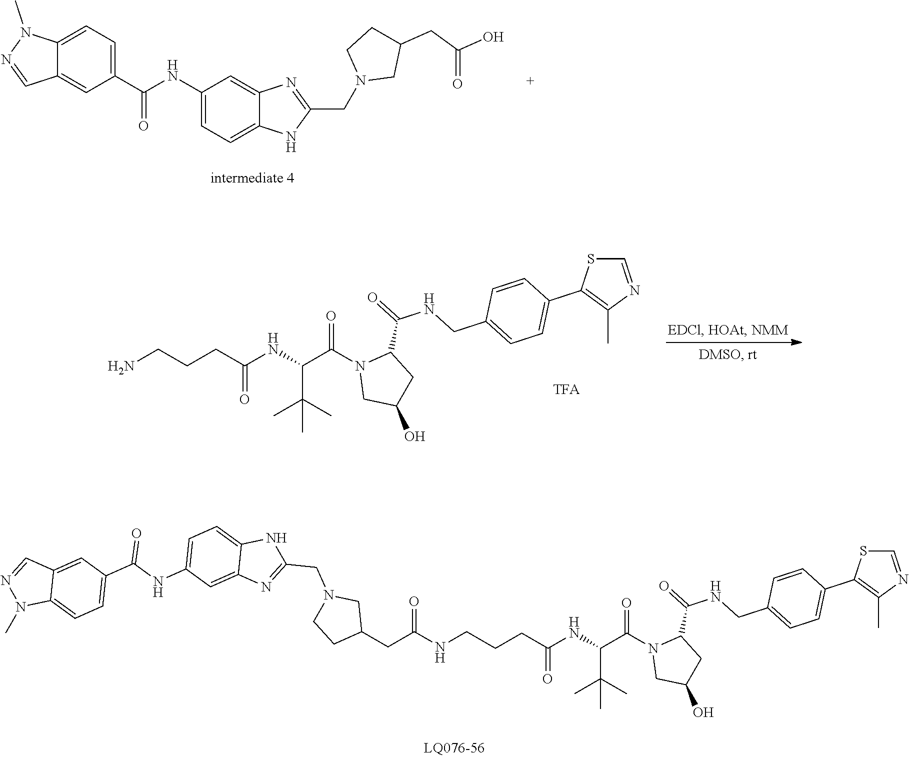

LQ076-56 was synthesized following the standard procedure for preparing LQ076-46 from intermediate 4 (12 mg, 0.02 mmol), (2S,4R)-1-((S)-2-(4-aminobutanamido)-3,3-dimethylbutanoyl)-4-hydroxy-N-(4-(4-methylthiazol-5-yl)benzyl)pyrrolidine-2-carboxamide (14.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-56 was obtained as white solid in TFA salt form (16.9 mg, 73%). ¹H NMR (600 MHz, Methanol-d₄) δ 9.07-9.05 (m, 1H), 8.47 (s, 1H), 8.33 (d, J=1.8 Hz, 1H), 8.18 (s, 1H), 8.05 (dd, J=8.8, 1.7 Hz, 1H), 7.69 (dd, J=8.9, 5.1 Hz, 2H), 7.61-7.59 (m, 1H), 7.48-7.39 (m, 4H), 4.77 (s, 2H), 4.65 (d, J=18.6 Hz, 1H), 4.61-4.57 (m, 1H), 4.53-4.48 (m, 2H), 4.38 (dd, J=15.5, 4.5 Hz, 1H), 4.14 (s, 3H), 3.95-3.91 (m, 1H), 3.85-3.61 (m, 3H), 3.59-3.52 (m, 1H), 3.25-3.19 (m, 2H), 2.86-2.80 (m, 1H), 2.55-2.41 (m, 5H), 2.39-2.22 (m, 4H), 2.12-2.07 (m, 1H), 1.91-1.76 (m, 3H), 1.03 (s, 9H). HRMS m/z [M+H]⁺ calcd for C₄₉H₆₀N₁₁O₆S⁺ 930.4443, found 930.4528.

### Example 13

### Synthesis of LQ076-57

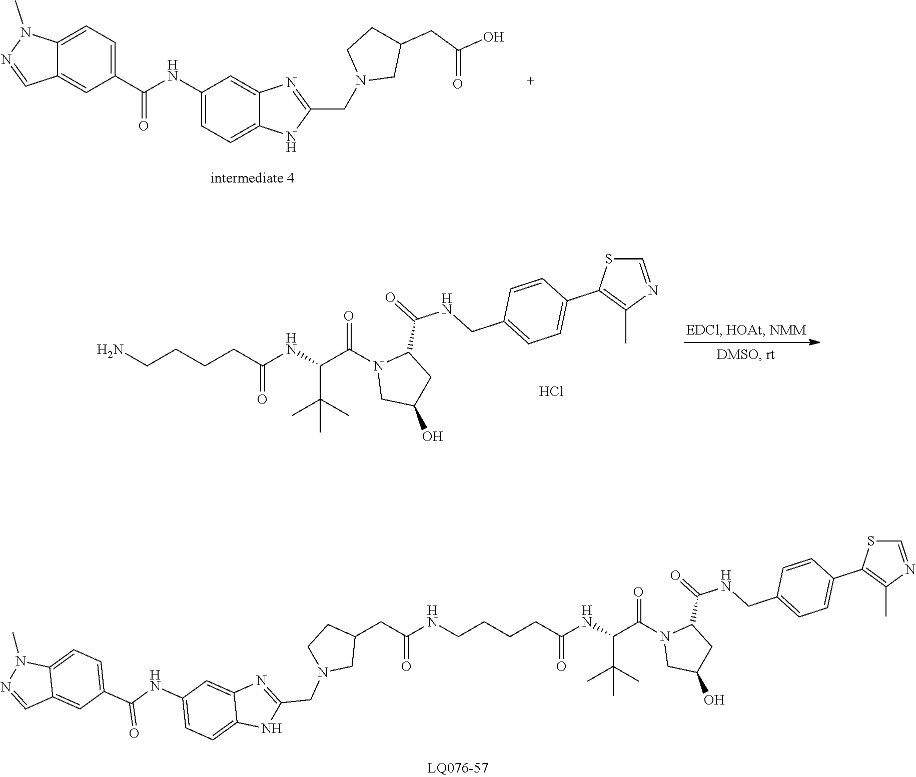

LQ076-57 was synthesized following the standard procedure for preparing LQ076-46 from intermediate 4 (12 mg, 0.02 mmol), (2S,4R)-1-((S)-2-(5-aminopentanamido)-3,3-dimethylbutanoyl)-4-hydroxy-N-(4-(4-methylthiazol-5-yl)benzyl)pyrrolidine-2-carboxamide (10.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-57 was obtained as white solid in TFA salt form (16.2 mg, 69%). ¹H NMR (600 MHz, Methanol-d₄) δ 9.05 (s, 1H), 8.47 (s, 1H), 8.33 (s, 1H), 8.18 (s, 1H), 8.05 (d, J=8.8 Hz, 1H), 7.71-7.67 (m, 2H), 7.61-7.58 (m, 1H), 7.49-7.40 (m, 4H), 4.77 (s, 2H), 4.64-4.49 (m, 4H), 4.38 (d, J=15.4 Hz, 1H), 4.14 (s, 3H), 3.91 (d, J=11.0 Hz, 1H), 3.83-3.75 (m, 2H), 3.67-3.54 (m, 2H), 3.32-3.27 (m, 1H), 3.22-3.16 (m, 2H), 2.87-2.81 (m, 1H), 2.51-2.46 (m, 4H), 2.44-2.21 (m, 6H), 2.12-2.06 (m, 1H), 1.88-1.81 (m, 1H), 1.66-1.58 (m, 2H), 1.55-1.49 (m, 2H), 1.04 (s, 9H). HRMS m/z [M+H]⁺ calcd for C₅₀H₆₂N₁₁O₆S⁺ 944.4600, found 944.4664.

### Example 14

### Synthesis of LQ076-58

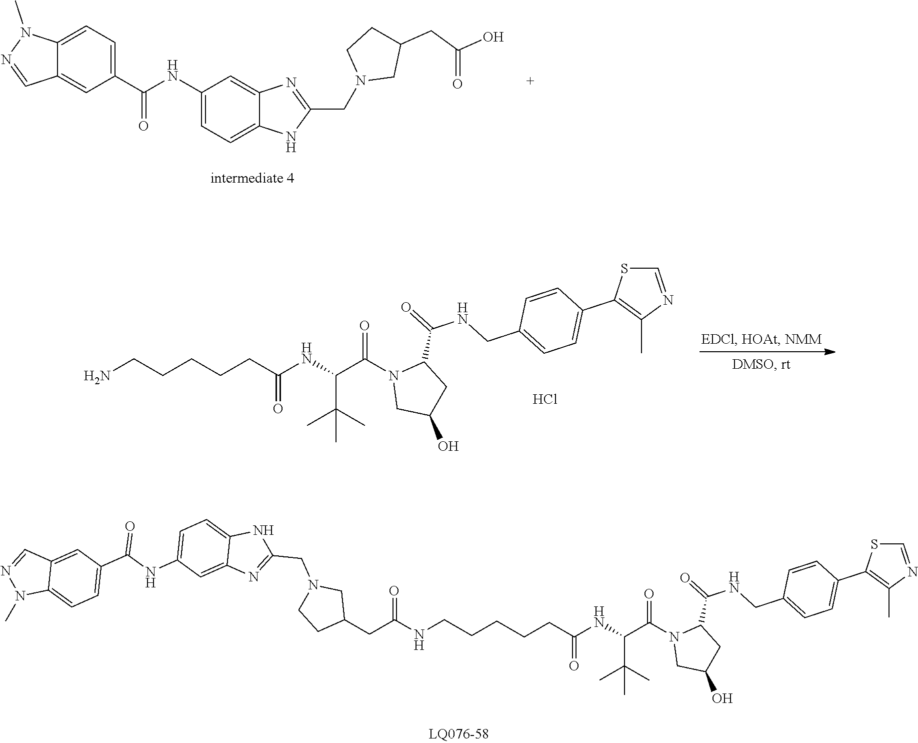

LQ076-58 was synthesized following the standard procedure for preparing LQ076-46 from intermediate 4 (12 mg, 0.02 mmol), (2S,4R)-1-((S)-2-(6-aminohexanamido)-3,3-dimethylbutanoyl)-4-hydroxy-N-(4-(4-methylthiazol-5-yl)benzyl)pyrrolidine-2-carboxamide (11.6 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-58 was obtained as white solid in TFA salt form (18.3 mg, 77%). ¹H NMR (600 MHz, Methanol-d₄) δ 9.03 (s, 1H), 8.47 (s, 1H), 8.33 (d, J=8.3 Hz, 1H), 8.18 (s, 1H), 8.07-8.03 (m, 1H), 7.70-7.67 (m, 2H), 7.59 (d, J=8.8 Hz, 1H), 7.49-7.40 (m, 4H), 4.75 (s, 2H), 4.64 (d, J=2.7 Hz, 1H), 4.62-4.57 (m, 1H), 4.55-4.49 (m, 2H), 4.38 (d, J=15.4 Hz, 1H), 4.14 (s, 3H), 3.92 (d, J=11.0 Hz, 1H), 3.83-3.80 (m, 1H), 3.76 (t, J=9.9 Hz, 1H), 3.67-3.60 (m, 1H), 3.59-3.53 (m, 1H), 3.30-3.25 (m, 1H), 3.17 (t, J=7.0 Hz, 2H), 2.85-2.78 (m, 1H), 2.51-2.46 (m, 4H), 2.44-2.21 (m, 5H), 2.12-2.07 (m, 1H), 1.88-1.80 (m, 1H), 1.66-1.59 (m, 2H), 1.55-1.48 (m, 2H), 1.38-1.31 (m, 2H), 1.04 (s, 9H). HRMS m/z [M+H]⁺ calcd for C₅₁H₆₄N₁₁O₆S⁺ 958.4756, found 958.4768.

### Example 15

### Synthesis of LQ076-59

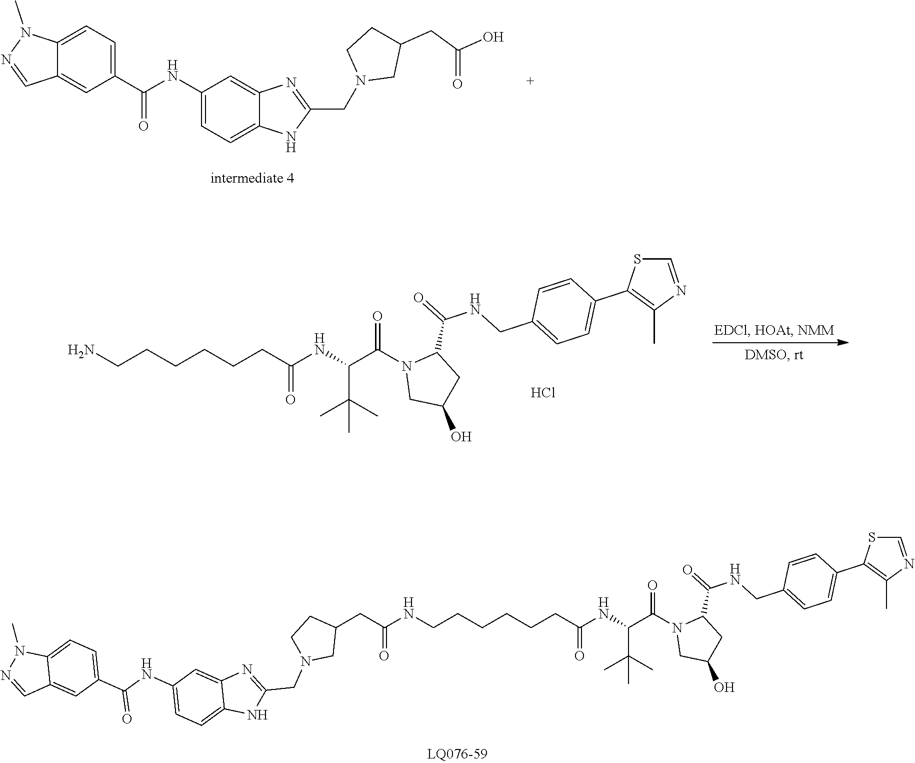

LQ076-59 was synthesized following the standard procedure for preparing LQ076-46 from intermediate 4 (12 mg, 0.02 mmol), (2S,4R)-1-((S)-2-(7-aminoheptanamido)-3,3-dimethylbutanoyl)-4-hydroxy-N-(4-(4-methylthiazol-5-yl)benzyl)pyrrolidine-2-carboxamide (11.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-59 was obtained as white solid in TFA salt form (19.2 mg, 80%). ¹H NMR (600 MHz, Methanol-d₄) δ 9.00 (s, 1H), 8.47 (d, J=1.5 Hz, 1H), 8.32 (d, J=6.5 Hz, 1H), 8.18 (s, 1H), 8.05 (dd, J=8.9, 1.7 Hz, 1H), 7.71-7.66 (m, 2H), 7.58 (dd, J=8.8, 1.9 Hz, 1H), 7.49-7.40 (m, 4H), 4.73 (s, 2H), 4.64 (d, J=1.7 Hz, 1H), 4.61-4.57 (m, 1H), 4.55-4.49 (m, 2H), 4.40-4.36 (m, 1H), 4.14 (s, 3H), 3.92 (d, J=10.9 Hz, 1H), 3.81 (dd, J=11.0, 3.9 Hz, 1H), 3.78-3.73 (m, 1H), 3.66-3.60 (m, 1H), 3.59-3.54 (m, 1H), 3.29-3.25 (m, 1H), 3.17 (t, J=7.0 Hz, 2H), 2.85-2.79 (m, 1H), 2.51-2.46 (m, 4H), 2.44-2.21 (m, 5H), 2.12-2.06 (m, 1H), 1.88-1.81 (m, 1H), 1.64-1.57 (m, 2H), 1.52-1.47 (m, 2H), 1.38-1.31 (m, 4H), 1.04 (s, 9H). HRMS m/z [M+H]⁺ calcd for C₅₂H₆₆N₁₁O₆S⁺ 972.4913, found 972.4952.

### Example 16

### Synthesis of LQ076-60

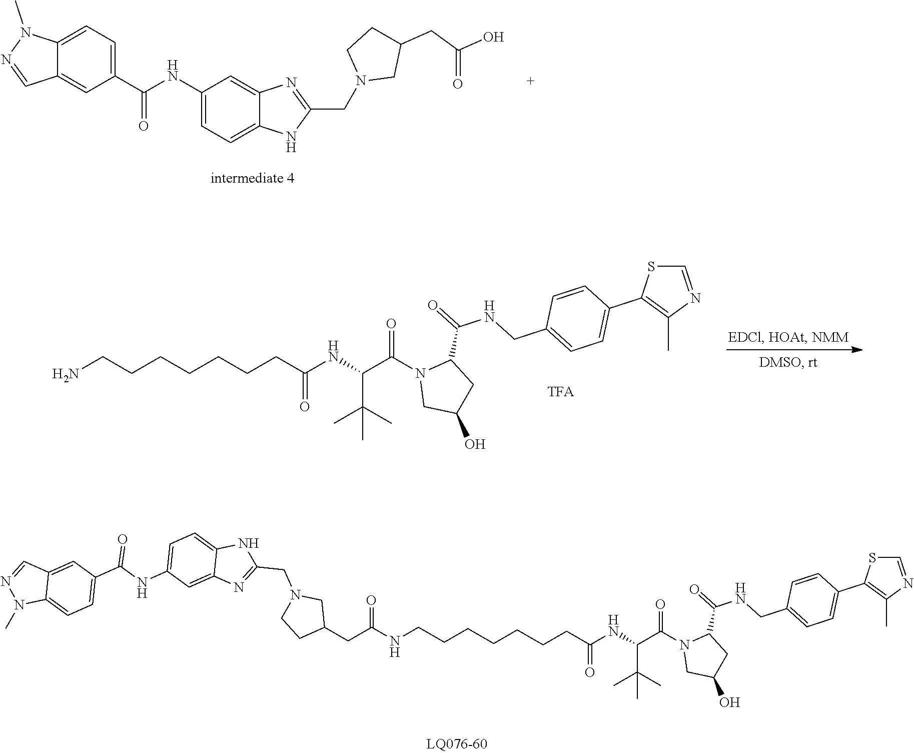

LQ076-60 was synthesized following the standard procedure for preparing LQ076-46 from intermediate 4 (12 mg, 0.02 mmol), (2S,4R)-1-((S)-2-(8-aminooctanamido)-3,3-dimethylbutanoyl)-4-hydroxy-N-(4-(4-methylthiazol-5-yl)benzyl)pyrrolidine-2-carboxamide (15.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-60 was obtained as white solid in TFA salt form (18 mg, 74%). ¹H NMR (600 MHz, Methanol-d₄) δ 9.04 (s, 1H), 8.47 (d, J=1.5 Hz, 1H), 8.33 (s, 1H), 8.18 (s, 1H), 8.05 (dd, J=8.9, 1.7 Hz, 1H), 7.70-7.67 (m, 2H), 7.60 (dd, J=8.7, 1.9 Hz, 1H), 7.49-7.41 (m, 4H), 4.76 (s, 2H), 4.65-4.63 (m, 1H), 4.61-4.55 (m, 1H), 4.54-4.49 (m, 2H), 4.37 (d, J=15.5 Hz, 1H), 4.13 (s, 3H), 3.92 (d, J=11.0 Hz, 1H), 3.83-3.73 (m, 2H), 3.67-3.54 (m, 2H), 3.30-3.26 (m, 1H), 3.17 (t, J=7.1 Hz, 2H), 2.86-2.80 (m, 1H), 2.51-2.45 (m, 4H), 2.44-2.21 (m, 5H), 2.12-2.06 (m, 1H), 1.88-1.81 (m, 1H), 1.64-1.55 (m, 2H), 1.53-1.45 (m, 2H), 1.37-1.28 (m, 6H), 1.04 (s, 9H). HRMS m/z [M+H]⁺ calcd for C₅₃H₆₈N₁₁O₆S⁺ 986.5069, found 986.5115.

### Example 17

### Synthesis of LQ076-61

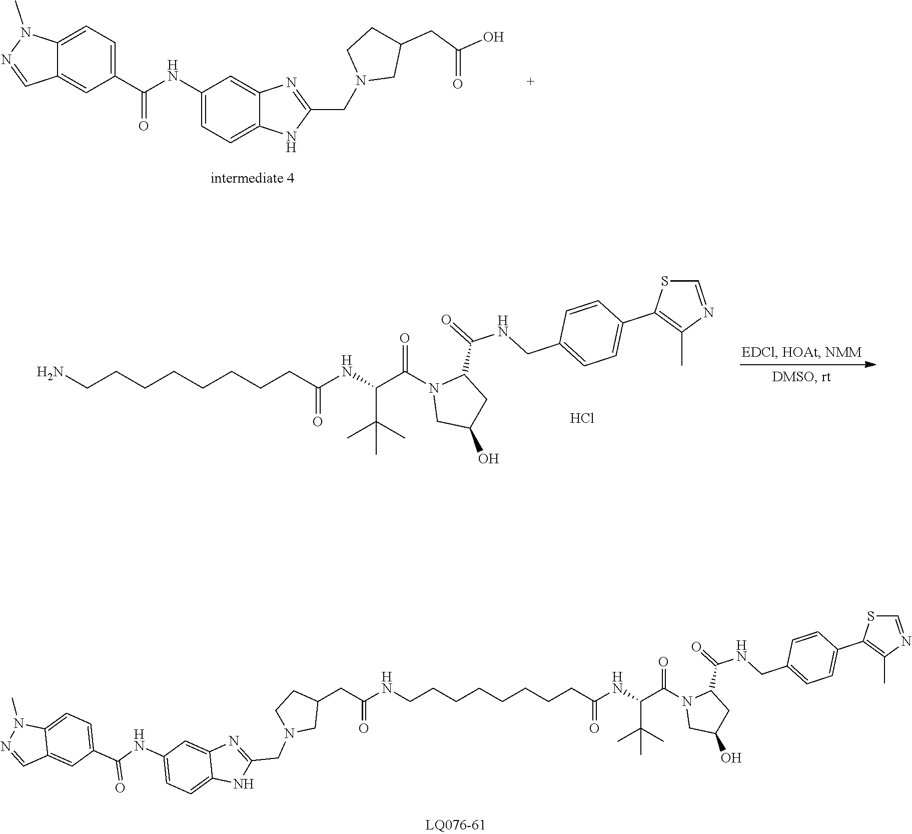

LQ076-61 was synthesized following the standard procedure for preparing LQ076-46 from intermediate 4 (12 mg, 0.02 mmol), (2S,4R)-1-((S)-2-(9-aminononanamido)-3,3-dimethylbutanoyl)-4-hydroxy-N-(4-(4-methylthiazol-5-yl)benzyl)pyrrolidine-2-carboxamide (12.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-61 was obtained as white solid in TFA salt form (19.1 mg, 78%). ¹H NMR (600 MHz, Methanol-d₄) δ 8.97 (s, 1H), 8.47 (s, 1H), 8.30 (s, 1H), 8.18 (s, 1H), 8.05 (dd, J=8.8, 1.7 Hz, 1H), 7.71-7.65 (m, 2H), 7.57 (dd, J=8.8, 1.9 Hz, 1H), 7.49-7.40 (m, 4H), 4.72 (s, 2H), 4.66-4.63 (m, 1H), 4.61-4.57 (m, 1H), 4.56-4.49 (m, 2H), 4.37 (d, J=15.5 Hz, 1H), 4.13 (s, 3H), 3.94-3.89 (m, 1H), 3.81 (dd, J=11.0, 3.9 Hz, 1H), 3.75 (t, J=9.9 Hz, 1H), 3.66-3.53 (m, 2H), 3.31-3.25 (m, 1H), 3.16 (t, J=7.1 Hz, 2H), 2.86-2.79 (m, 1H), 2.51-2.45 (m, 4H), 2.44-2.39 (m, 1H), 2.38-2.20 (m, 4H), 2.12-2.06 (m, 1H), 1.88-1.80 (m, 1H), 1.64-1.55 (m, 2H), 1.52-1.46 (m, 2H), 1.36-1.27 (m, 8H), 1.04 (s, 9H). HRMS m/z [M+H]⁺ calcd for C₅₄H₇₀N₁₁O₆S⁺ 1000.5226, found 1000.5241.

### Example 18

### Synthesis of LQ076-62

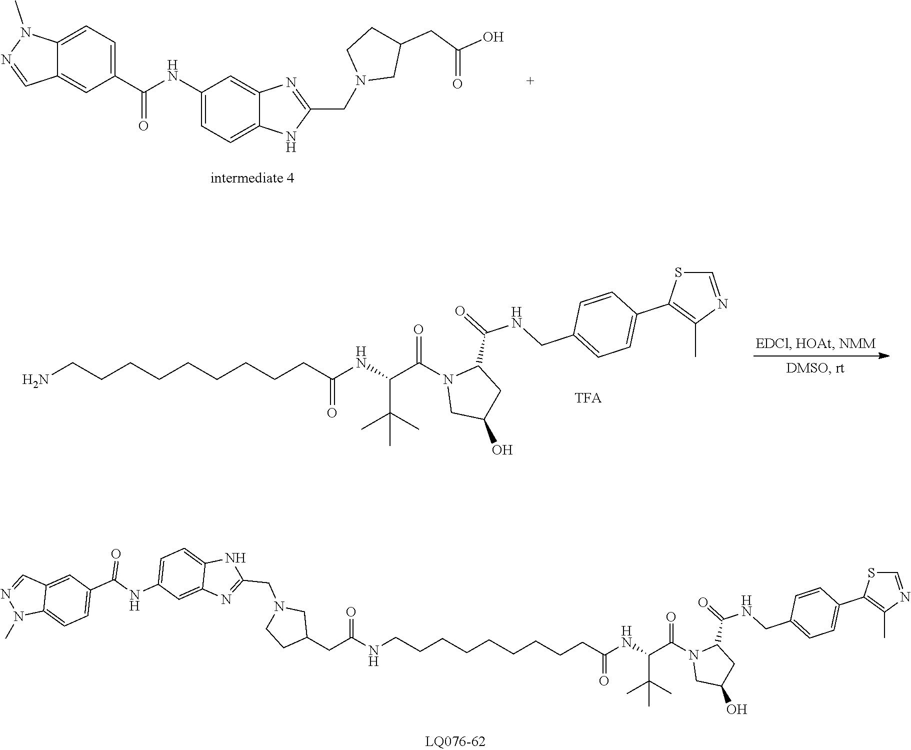

LQ076-62 was synthesized following the standard procedure for preparing LQ076-46 from intermediate 4 (12 mg, 0.02 mmol), (2S,4R)-1-((S)-2-(10-aminodecanamido)-3,3-dimethylbutanoyl)-4-hydroxy-N-(4-(4-methylthiazol-5-yl)benzyl)pyrrolidine-2-carboxamide (16.6 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-62 was obtained as white solid in TFA salt form (15.6 mg, 63%). ¹H NMR (800 MHz, Methanol-d₄) δ 8.96 (s, 1H), 8.47 (s, 1H), 8.30 (s, 1H), 8.19 (s, 1H), 8.06 (d, J=8.8 Hz, 1H), 7.70-7.66 (m, 2H), 7.58 (d, J=8.7 Hz, 1H), 7.49-7.41 (m, 4H), 4.73 (s, 2H), 4.66-4.64 (m, 1H), 4.62-4.58 (m, 1H), 4.56-4.50 (m, 2H), 4.38 (d, J=15.4 Hz, 1H), 4.14 (s, 3H), 3.92 (d, J=11.0 Hz, 1H), 3.82 (dd, J=10.9, 4.0 Hz, 1H), 3.78-3.74 (m, 1H), 3.66-3.54 (m, 2H), 3.31-3.26 (m, 1H), 3.17 (t, J=7.1 Hz, 2H), 2.86-2.80 (m, 1H), 2.51-2.46 (m, 4H), 2.44-2.40 (m, 1H), 2.38-2.33 (m, 1H), 2.32-2.28 (m, 1H), 2.27-2.21 (m, 2H), 2.13-2.08 (m, 1H), 1.88-1.82 (m, 1H), 1.64-1.56 (m, 2H), 1.52-1.46 (m, 2H), 1.36-1.26 (m, 12H), 1.05 (s, 9H). HRMS m/z [M+H]⁺ calcd for C₅₅H₇₂N₁₁O₆S⁺ 1014.5382, found 1014.5252.

### Example 19

### Synthesis of LQ076-63

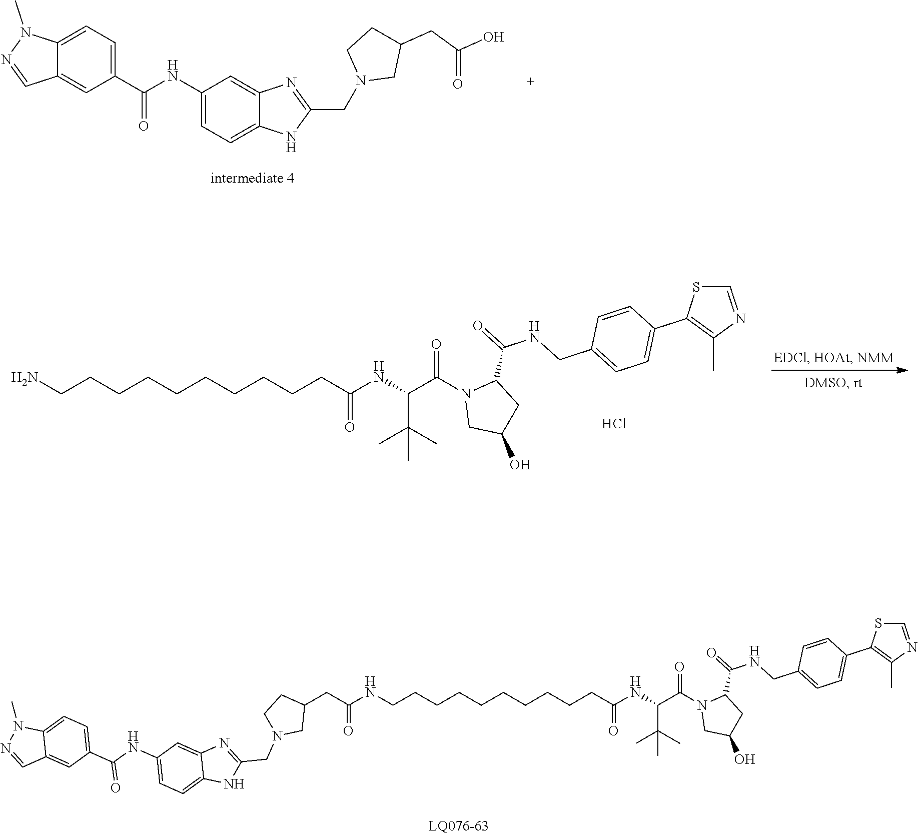

LQ076-63 was synthesized following the standard procedure for preparing LQ076-46 from intermediate 4 (12 mg, 0.02 mmol), (2S,4R)-1-((S)-2-(11-aminoundecanamido)-3,3-dimethylbutanoyl)-4-hydroxy-N-(4-(4-methylthiazol-5-yl)benzyl)pyrrolidine-2-carboxamide (13 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-63 was obtained as white solid in TFA salt form (18.3 mg, 73%). ¹H NMR (600 MHz, Methanol-d₄) δ 9.07 (s, 1H), 8.48-8.46 (m, 1H), 8.34 (d, J=2.0 Hz, 1H), 8.18 (s, 1H), 8.05 (dd, J=8.9, 1.7 Hz, 1H), 7.71-7.67 (m, 2H), 7.62 (dd, J=8.8, 2.0 Hz, 1H), 7.50-7.41 (m, 4H), 4.78 (s, 2H), 4.66-4.64 (m, 1H), 4.61-4.55 (m, 1H), 4.54-4.49 (m, 2H), 4.37 (d, J=15.6 Hz, 1H), 4.13 (s, 3H), 3.92 (d, J=11.0 Hz, 1H), 3.81 (dd, J=10.9, 4.0 Hz, 1H), 3.79-3.74 (m, 1H), 3.68-3.54 (m, 2H), 3.31-3.27 (m, 1H), 3.16 (t, J=7.1 Hz, 2H), 2.86-2.80 (m, 1H), 2.51-2.46 (m, 4H), 2.44-2.20 (m, 5H), 2.12-2.06 (m, 1H), 1.89-1.81 (m, 1H), 1.64-1.55 (m, 2H), 1.52-1.44 (m, 2H), 1.35-1.25 (m, 12H), 1.05 (s, 9H). HRMS m/z [M+H]⁺ calcd for C₅₆H₇₄N₁₁O₆S⁺ 1028.5539, found 1028.5552.

### Example 20

### Synthesis of LQ076-64

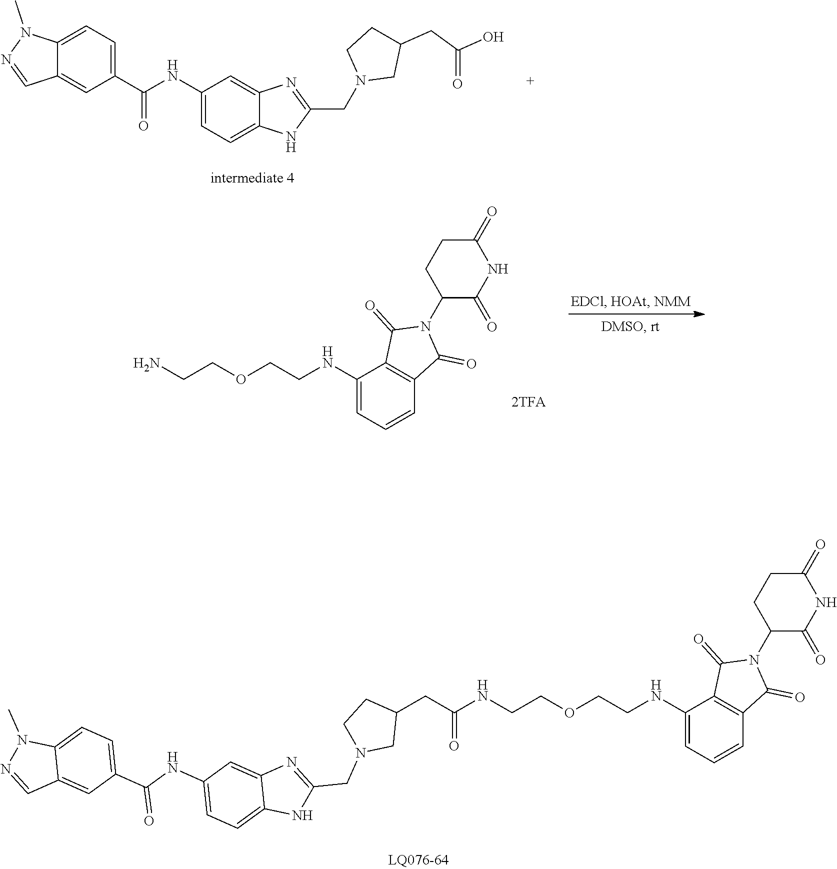 [SMILES: Cn1ncc2cc(C(=O)Nc3ccc4[nH]c(CN5CCC(CC(=O)O)C5)nc4c3)ccc21 ; NCCOCCNc1cccc2c1C(=O)N(C1CCC(=O)NC1=O)C2=O ; "2TFA" ; Cn1ncc2cc(C(=O)Nc3ccc4[nH]c(CN5CCC(CC(=O)NCCOCCNc6cccc7c6C(=O)N(C6CCC(=O)NC6=O)C7=O)C5)nc4c3)ccc21]

LQ076-64 was synthesized following the standard procedure for preparing LQ076-46 from intermediate 4 (12 mg, 0.02 mmol), 4-((2-(2-aminoethoxy)ethyl)amino)-2-(2,6-dioxopiperidin-3-yl)isoindoline-1,3-dione (9.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-64 was obtained as yellow solid in TFA salt form (13.2 mg, 66%). ¹H NMR (600 MHz, Methanol-d₄) δ 8.45 (dd, J=1.7, 0.8 Hz, 1H), 8.26 (s, OH), 8.18 (d, J=0.9 Hz, 1H), 8.04 (dd, J=8.8, 1.7 Hz, 1H), 7.68 (d, J=8.6 Hz, 1H), 7.66-7.63 (m, 1H), 7.55-7.51 (m, 2H), 7.06-7.01 (m, 2H), 5.08-5.03 (m, 1H), 4.68 (s, 2H), 4.14 (s, 3H), 3.76-3.70 (m, 1H), 3.70-3.64 (m, 2H), 3.62-3.49 (m, 4H), 3.47-3.36 (m, 4H), 3.29-3.21 (m, 1H), 2.91-2.65 (m, 4H), 2.54-2.42 (m, 1H), 2.42-2.36 (m, 1H), 2.34-2.26 (m, 1H), 2.16-2.08 (m, 1H), 1.89-1.77 (m, 1H). HRMS m/z [M+H]⁺ calcd for C₄₀H₄₃N₁₀O₇⁺ 775.3311, found 775.3346.

### Example 21

### Synthesis of LQ076-65

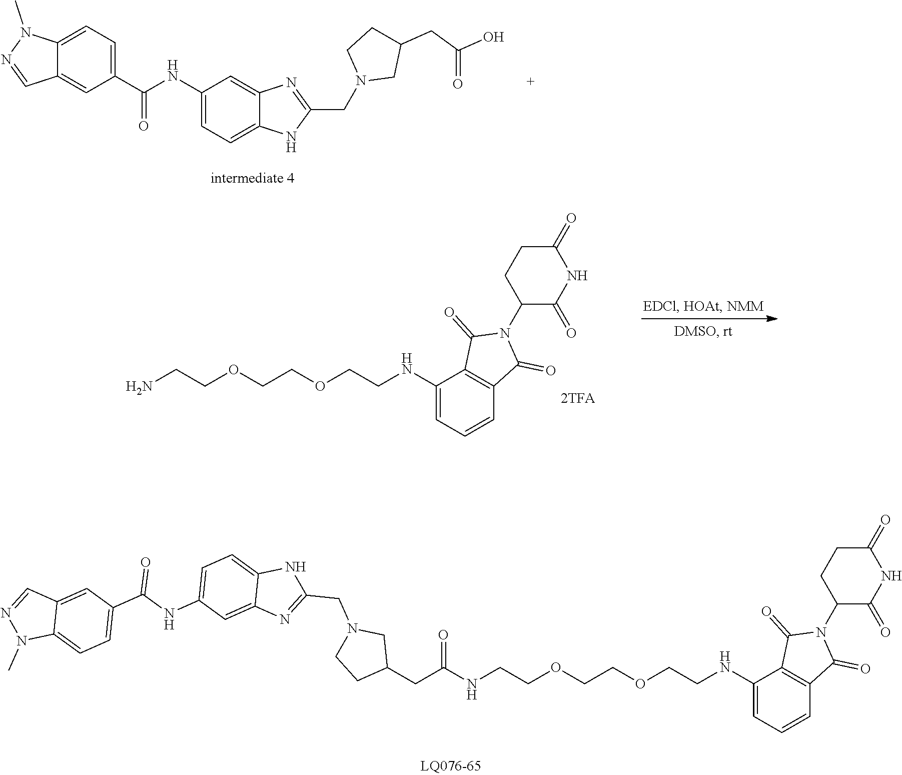 [SMILES: Cn1ncc2cc(C(=O)Nc3ccc4[nH]c(CN5CCC(CC(=O)O)C5)nc4c3)ccc21 ; NCCOCCOCCNc1cccc2c1C(=O)N(C1CCC(=O)NC1=O)C2=O ; Cn1ncc2cc(C(=O)Nc3ccc4[nH]c(CN5CCC(CC(=O)NCCOCCOCCNc6cccc7c6C(=O)N(C6CCC(=O)NC6=O)C7=O)C5)nc4c3)ccc21]

LQ076-65 was synthesized following the standard procedure for preparing LQ076-46 from intermediate 4 (12 mg, 0.02 mmol), 4-((2-(2-(2-aminoethoxy)ethoxy)ethyl)amino)-2-(2,6-dioxopiperidin-3-yl)isoindoline-1,3-dione (10.7 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-65 was obtained as yellow solid in TFA salt form (14 mg, 70%). ¹H NMR (600 MHz, Methanol-d₄) δ 8.45 (dd, J=1.7, 0.9 Hz, 1H), 8.27 (d, J=2.0 Hz, 1H), 8.17 (s, 1H), 8.06-8.01 (m, 1H), 7.69-7.63 (m, 2H), 7.55 (dd, J=8.7, 2.0 Hz, 1H), 7.51 (dd, J=8.5, 7.1 Hz, 1H), 7.03 (dd, J=11.9, 7.8 Hz, 2H), 5.08-5.03 (m, 1H), 4.70 (s, 2H), 4.13 (s, 3H), 3.76-3.68 (m, 3H), 3.66-3.58 (m, 5H), 3.58-3.51 (m, 3H), 3.47 (t, J=5.2 Hz, 2H), 3.39-3.34 (m, 2H), 3.30-3.24 (m, 1H), 2.89-2.65 (m, 4H), 2.50-2.42 (m, 1H), 2.42-2.36 (m, 1H), 2.36-2.26 (m, 1H), 2.15-2.07 (m, 1H), 1.87-1.77 (m, 1H). HRMS m/z [M+H]⁺ calcd for C₄₂H₄₇N₁₀O₈⁺ 819.3573, found 819.3590.

### Example 22

### Synthesis of LQ076-66

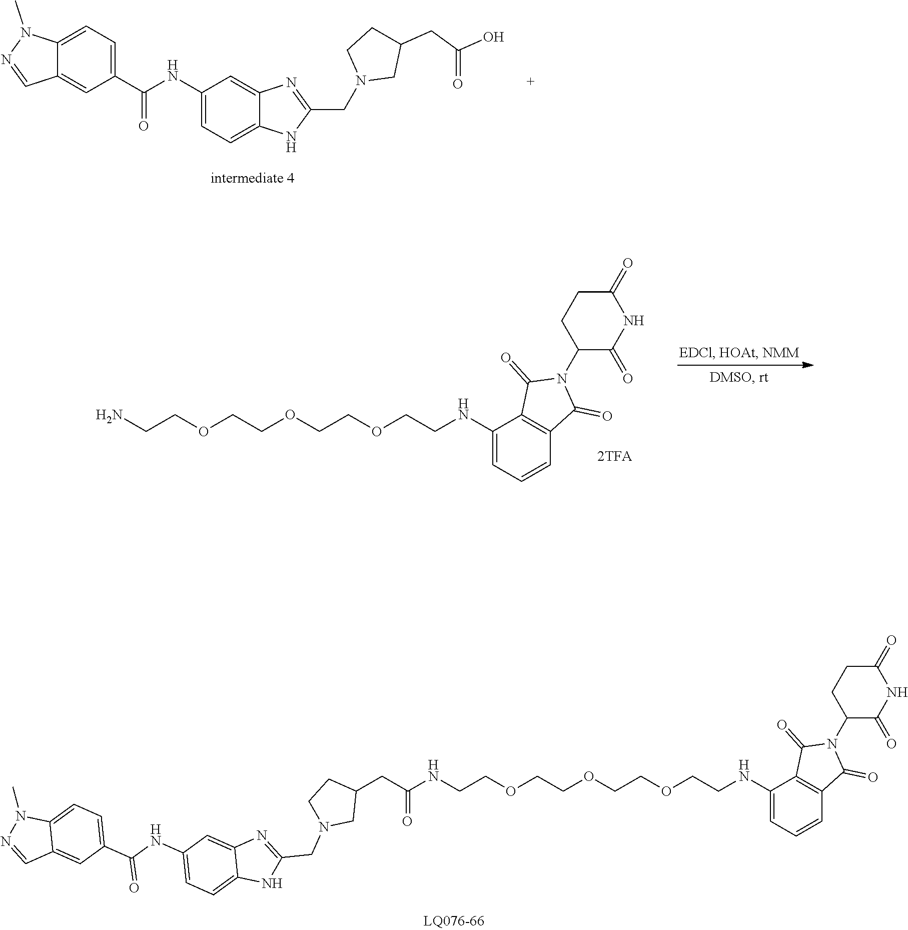 [SMILES: Cn1ncc2cc(C(=O)Nc3ccc4[nH]c(CN5CCC(CC(=O)O)C5)nc4c3)ccc21 ; NCCOCCOCCOCCNc1cccc2c1C(=O)N(C1CCC(=O)NC1=O)C2=O ; Cn1ncc2cc(C(=O)Nc3ccc4[nH]c(CN5CCC(CC(=O)NCCOCCOCCOCCNc6cccc7c6C(=O)N(C6CCC(=O)NC6=O)C7=O)C5)nc4c3)ccc21]

LQ076-66 was synthesized following the standard procedure for preparing LQ076-46 from intermediate 4 (12 mg, 0.02 mmol), 4-((2-(2-(2-(2-aminoethoxy)ethoxy)ethoxy)ethyl)amino)-2-(2,6-dioxopiperidin-3-yl)isoindoline-1,3-dione (11.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-66 was obtained as yellow solid in TFA salt form (14.6 mg, 67%).

¹H NMR (600 MHz, Methanol-d₄) δ 8.47-8.44 (m, 1H), 8.27 (d, J=1.9 Hz, 1H), 8.17 (d, J=0.9 Hz, 1H), 8.04 (dd, J=8.8, 1.7 Hz, 1H), 7.70-7.63 (m, 2H), 7.56 (dd, J=8.7, 2.0 Hz, 1H), 7.51 (dd, J=8.6, 7.1 Hz, 1H), 7.05-7.00 (m, 2H), 5.05 (dd, J=12.8, 5.5 Hz, 1H), 4.71 (s, 2H), 4.13 (s, 3H), 3.76-3.68 (m, 4H), 3.67-3.60 (m, 8H), 3.59-3.54 (m, 3H), 3.51 (t, J=5.4 Hz, 2H), 3.46 (t, J=5.2 Hz, 2H), 3.38-3.34 (m, 2H), 3.31-3.26 (m, 1H), 2.89-2.77 (m, 2H), 2.76-2.65 (m, 2H), 2.48 (dd, J=15.0, 6.1 Hz, 1H), 2.41 (dd, J=15.0, 7.9 Hz, 1H), 2.37-2.28 (m, 1H), 2.15-2.07 (m, 1H), 1.88-1.79 (m, 1H). HRMS m/z [M+H]⁺ calcd for C₄₄H₅₁N₁₀O₉⁺ 863.3835, found 863.3878.

### Example 23

### Synthesis of LQ076-67

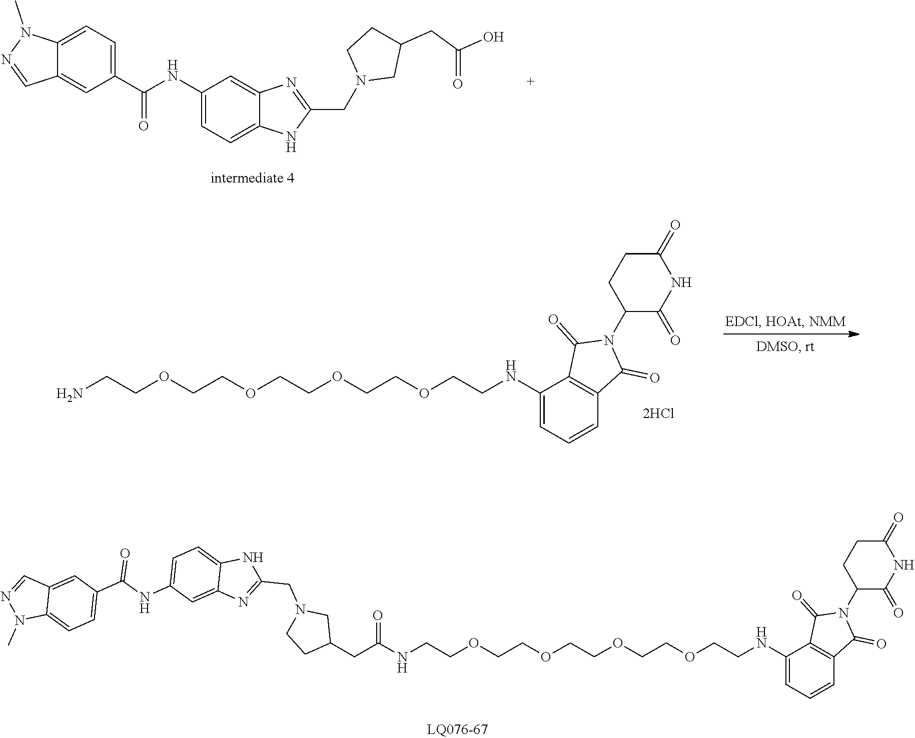 [SMILES: Cn1ncc2cc(C(=O)Nc3ccc4[nH]c(CN5CCC(CC(=O)O)C5)nc4c3)ccc21 ; NCCOCCOCCOCCOCCNc1cccc2c1C(=O)N(C1CCC(=O)NC1=O)C2=O ; Cn1ncc2cc(C(=O)Nc3ccc4[nH]c(CN5CCC(CC(=O)NCCOCCOCCOCCOCCNc6cccc7c6C(=O)N(C6CCC(=O)NC6=O)C7=O)C5)nc4c3)ccc21]

LQ076-67 was synthesized following the standard procedure for preparing LQ076-46 from intermediate 4 (12 mg, 0.02 mmol), 4-((14-amino-3,6,9,12-tetraoxatetradecyl)amino)-2-(2,6-dioxopiperidin-3-yl)isoindoline-1,3-dione (11.4 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-67 was obtained as yellow solid in TFA salt form (15.6 mg, 70%). ¹H NMR (600 MHz, Methanol-d₄) δ 8.46 (dd, J=1.7, 0.8 Hz, 1H), 8.28 (d, J=2.0 Hz, 1H), 8.18 (d, J=0.9 Hz, 1H), 8.05 (dd, J=8.8, 1.7 Hz, 1H), 7.71-7.63 (m, 2H), 7.56-7.50 (m, 2H), 7.06-7.02 (m, 2H), 5.05 (dd, J=12.8, 5.5 Hz, 1H), 4.69 (s, 2H), 4.14 (s, 3H), 3.79-3.69 (m, 3H), 3.67-3.58 (m, 11H), 3.58-3.50 (m, 5H), 3.47 (t, J=5.2 Hz, 2H), 3.40-3.34 (m, 2H), 3.31-3.24 (m, 1H), 2.90-2.66 (m, 4H), 2.50 (dd, J=15.0, 6.1 Hz, 1H), 2.42 (dd, J=15.0, 7.9 Hz, 1H), 2.38-2.29 (m, 1H), 2.15-2.08 (m, 1H), 1.89-1.79 (m, 1H). HRMS m/z [M+H]⁺ calcd for C₄₆H₅₅N₁₀O₁₀⁺ 907.4097, found 907.4127.

### Example 24

### Synthesis LQ076-68

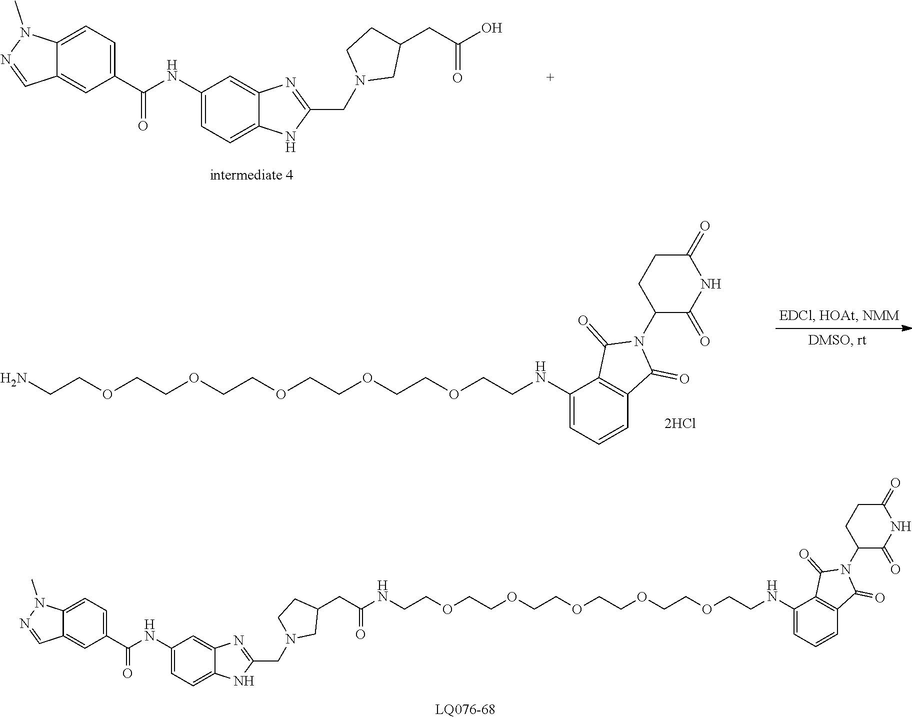 [SMILES: Cn1ncc2cc(C(=O)Nc3ccc4[nH]c(CN5CCC(CC(=O)O)C5)nc4c3)ccc21 ; NCCOCCOCCOCCOCCOCCNc1cccc2c1C(=O)N(C1CCC(=O)NC1=O)C2=O ; Cn1ncc2cc(C(=O)Nc3ccc4[nH]c(CN5CCC(CC(=O)NCCOCCOCCOCCOCCOCCNc6cccc7c6C(=O)N(C6CCC(=O)NC6=O)C7=O)C5)nc4c3)ccc21]

dioxopiperidin-3-yl)isoindoline-1,3-dione (12.7 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-68 was obtained as yellow solid in TFA salt form (15 mg, 64%). ¹H NMR (600 MHz, Methanol-d₄) δ 8.46 (d, J=1.4 Hz, 1H), 8.35 (d, J=2.0 Hz, 1H), 8.18 (s, 1H), 8.04 (dd, J=8.9, 1.7 Hz, 1H), 7.71-7.66 (m, 2H), 7.61 (dd, J=8.8, 2.0 Hz, 1H), 7.51 (dd, J=8.6, 7.1 Hz, 1H), 7.04-7.00 (m, 2H), 5.05 (dd, J=12.8, 5.5 Hz, 1H), 4.80 (s, 2H), 4.13 (s, 3H), 3.72-3.49 (m, 23H), 3.45 (t, J=5.2 Hz, 2H), 3.40-3.34 (m, 2H), 3.32-3.26 (m, 1H), 2.89-2.82 (m, 2H), 2.77-2.67 (m, 2H), 2.50 (dd, J=15.0, 6.0 Hz, 1H), 2.42 (dd, J=15.0, 8.0 Hz, 1H), 2.38-2.31 (m, 1H), 2.14-2.09 (m, 1H), 1.89-1.82 (m, 1H). HRMS m/z [M+H]⁺ calcd for C₄₈H₅₉N₁₀O₁₁⁺ 951.4359, found 951.4397.

### Example 25

### Synthesis of LQ076-69

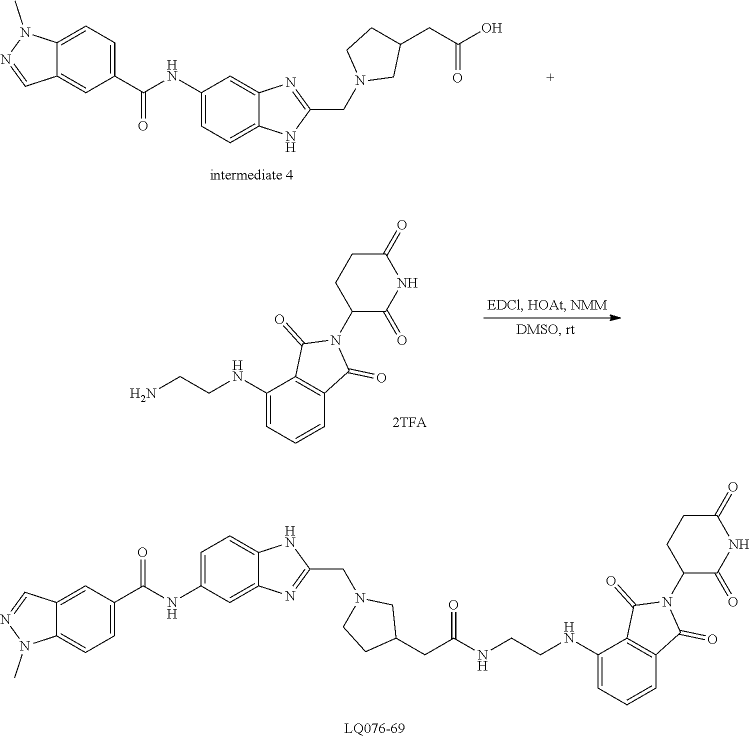 [SMILES: Cn1ncc2cc(C(=O)Nc3ccc4[nH]c(CN5CCC(CC(=O)O)C5)nc4c3)ccc21 ; NCCNc1cccc2c1C(=O)N(C1CCC(=O)NC1=O)C2=O ; Cn1ncc2cc(C(=O)Nc3ccc4[nH]c(CN5CCC(CC(=O)NCCNc6cccc7c6C(=O)N(C6CCC(=O)NC6=O)C7=O)C5)nc4c3)ccc21]

LQ076-69 was synthesized following the standard procedure for preparing LQ076-46 from intermediate 4 (12 mg, 0.02 mmol), 4-((2-aminoethyl)amino)-2-(2,6-dioxopiperidin-3-yl)isoindoline-1,3-dione (9.1 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-69 was obtained as yellow solid in TFA salt form (13 mg, 68%). ¹H NMR (600 MHz, Methanol-d₄) δ 8.45 (d, J=1.7 Hz, 1H), 8.29-8.27 (m, 1H), 8.17 (s, 1H), 8.04 (dd, J=8.9, 1.7 Hz, 1H), 7.69-7.65 (m, 2H), 7.57-7.51 (m, 2H), 7.09 (dd, J=8.6, 6.7 Hz, 1H), 7.05-7.01 (m, 1H), 5.06-5.02 (m, 1H), 4.76-4.71 (m, 2H), 4.13 (s, 3H), 3.75-3.70 (m, 1H), 3.63-3.57 (m, 1H), 3.55-3.43 (m, 5H), 3.31-3.27 (m, 1H), 2.88-2.64 (m, 4H), 2.51-2.28 (m, 3H), 2.12-2.06 (m, 1H), 1.84-1.75 (m, 1H). HRMS m/z [M+H]⁺ calcd for C₃₈H₃₉N₁₀O₆⁺ 731.3049, found 731.3080.

### Example 26

### Synthesis of LQ076-70

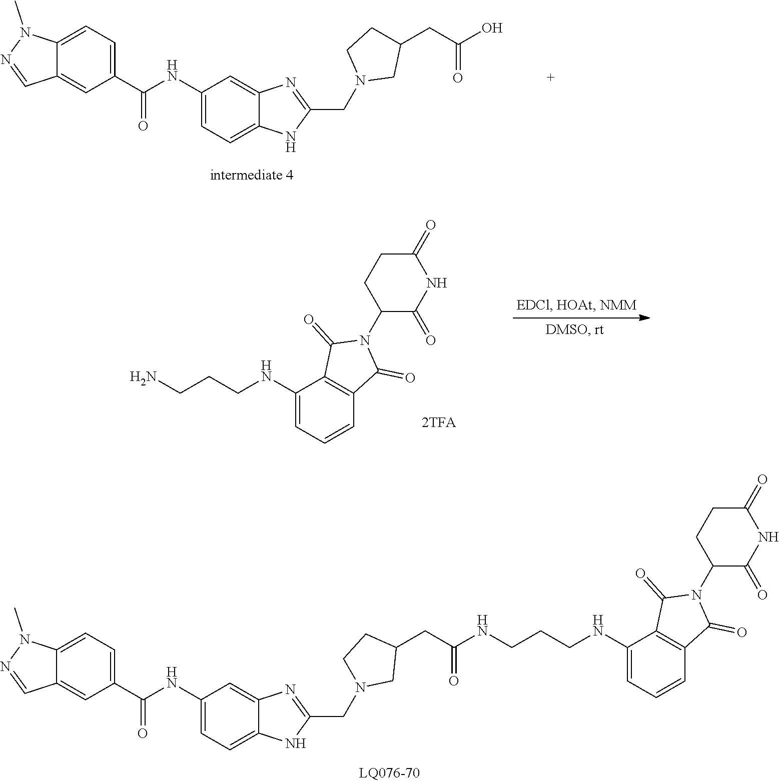 [SMILES: Cn1ncc2cc(C(=O)Nc3ccc4[nH]c(CN5CCC(CC(=O)O)C5)nc4c3)ccc21 ; NCCCNc1cccc2c1C(=O)N(C1CCC(=O)NC1=O)C2=O ; Cn1ncc2cc(C(=O)Nc3ccc4[nH]c(CN5CCC(CC(=O)NCCCNc6cccc7c6C(=O)N(C6CCC(=O)NC6=O)C7=O)C5)nc4c3)ccc21]

LQ076-70 was synthesized following the standard procedure for preparing LQ076-46 from intermediate 4 (12 mg, 0.02 mmol), 4-((3-aminopropyl)amino)-2-(2,6-dioxopiperidin-3-yl)isoindoline-1,3-dione (9.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-70 was obtained as yellow solid in TFA salt form (14.5 mg, 75%). ¹H NMR (600 MHz, Methanol-d₄) δ 8.46 (s, 1H), 8.33-8.30 (m, 1H), 8.19 (s, 1H), 8.04 (dd, J=8.8, 1.6 Hz, 1H), 7.71-7.65 (m, 2H), 7.58-7.54 (m, 1H), 7.53-7.49 (m, 1H), 7.02-6.99 (m, 2H), 5.05 (dd, J=12.4, 5.6 Hz, 1H), 4.77-4.73 (m, 2H), 4.14 (s, 3H), 3.76-3.70 (m, 1H), 3.68-3.62 (m, 1H), 3.59-3.52 (m, 1H), 3.37 (t, J=6.5 Hz, 2H), 3.34-3.33 (m, 2H), 3.32-3.30 (m, 1H), 2.88-2.80 (m, 2H), 2.77-2.67 (m, 2H), 2.54-2.42 (m, 3H), 2.38-2.32 (m, 1H), 2.13-2.07 (m, 1H), 1.86-1.80 (m, 3H). HRMS m/z [M+H]⁺ calcd for C₃₉H₄₁N₁₀O₆⁺ 745.3205, found 745.3233.

### Example 27

### Synthesis of LQ076-71

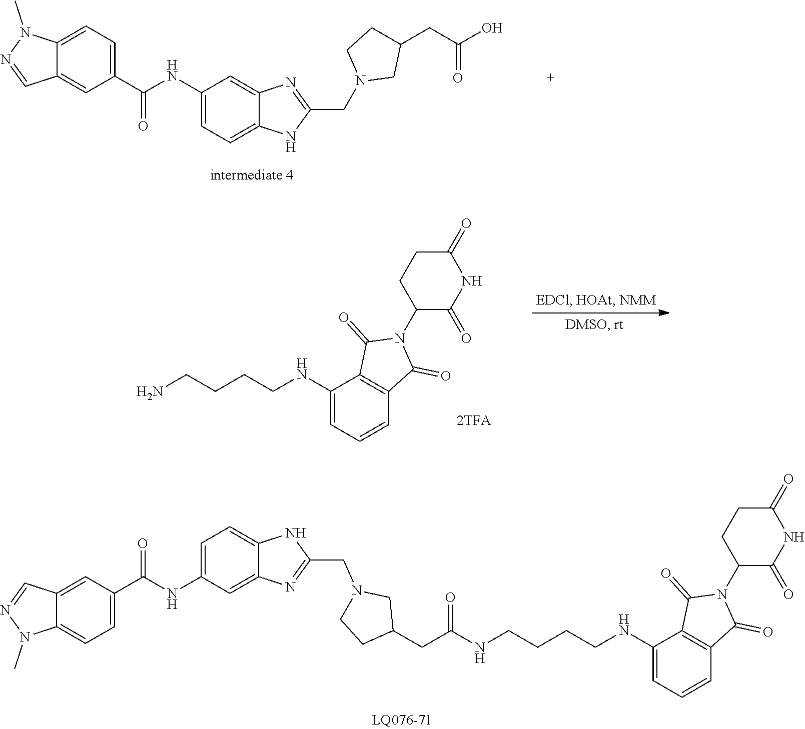 [SMILES: Cn1ncc2cc(C(=O)Nc3ccc4[nH]c(CN5CCC(CC(=O)O)C5)nc4c3)ccc21 ; NCCCCNc1cccc2c1C(=O)N(C1CCC(=O)NC1=O)C2=O ; Cn1ncc2cc(C(=O)Nc3ccc4[nH]c(CN5CCC(CC(=O)NCCCCNc6cccc7c6C(=O)N(C6CCC(=O)NC6=O)C7=O)C5)nc4c3)ccc21]

LQ076-71 was synthesized following the standard procedure for preparing LQ076-46 from intermediate 4 (12 mg, 0.02 mmol), 4-((4-aminobutyl)amino)-2-(2,6-dioxopiperidin-3-yl)isoindoline-1,3-dione (9.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-71 was obtained as yellow solid in TFA salt form (13.6 mg, 69%). ¹H NMR (600 MHz, Methanol-d₄) δ 8.45-8.43 (m, 1H), 8.30 (d, J=2.0 Hz, 1H), 8.17 (s, 1H), 8.03 (dd, J=8.8, 1.7 Hz, 1H), 7.68-7.65 (m, 2H), 7.56 (dd, J=8.8, 2.0 Hz, 1H), 7.50 (dd, J=8.6, 7.1 Hz, 1H), 7.01-6.97 (m, 2H), 5.03 (dd, J=12.8, 5.5 Hz, 1H), 4.74 (s, 2H), 4.13 (s, 3H), 3.77-3.71 (m, 1H), 3.65-3.59 (m, 1H), 3.57-3.51 (m, 1H), 3.32-3.27 (m, 3H), 3.26-3.21 (m, 2H), 2.87-2.79 (m, 2H), 2.76-2.65 (m, 2H), 2.50-2.45 (m, 1H), 2.41 (dd, J=15.0, 7.9 Hz, 1H), 2.35-2.30 (m, 1H), 2.12-2.07 (m, 1H), 1.86-1.78 (m, 1H), 1.69-1.58 (m, 4H). HRMS m/z [M+H]⁺ calcd for C₄₀H₄₃N₁₀O₆⁺ 759.3362, found 759.3369.

### Example 28

### Synthesis of LQ076-72

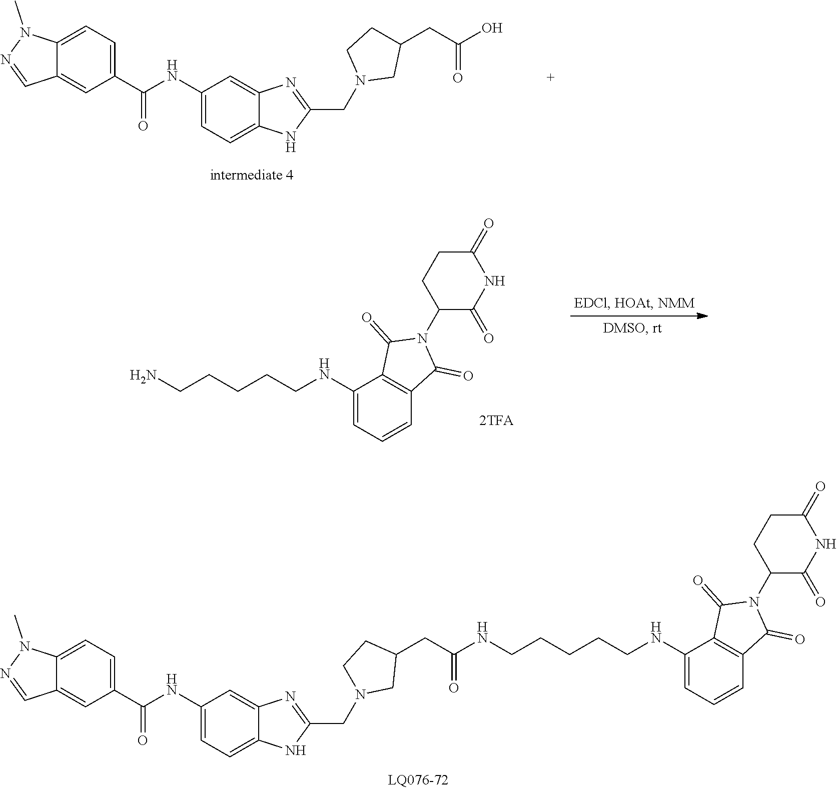 [SMILES: Cn1ncc2cc(C(=O)Nc3ccc4[nH]c(CN5CCC(CC(=O)O)C5)nc4c3)ccc21 ; NCCCCCNc1cccc2c1C(=O)N(C1CCC(=O)NC1=O)C2=O ; Cn1ncc2cc(C(=O)Nc3ccc4[nH]c(CN5CCC(CC(=O)NCCCCCNc6cccc7c6C(=O)N(C6CCC(=O)NC6=O)C7=O)C5)nc4c3)ccc21]

LQ076-72 was synthesized following the standard procedure for preparing LQ076-46 from intermediate 4 (12 mg, 0.02 mmol), 4-((5-aminopentyl)amino)-2-(2,6-dioxopiperidin-3-yl)isoindoline-1,3-dione (9.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-72 was obtained as yellow solid in TFA salt form (15.4 mg, 77%). ¹H NMR (600 MHz, Methanol-d₄) δ 8.45-8.43 (m, 1H), 8.30 (d, J=2.0 Hz, 1H), 8.17 (s, 1H), 8.03 (dd, J=8.8, 1.7 Hz, 1H), 7.68-7.65 (m, 2H), 7.56 (dd, J=8.8, 2.0 Hz, 1H), 7.50 (dd, J=8.6, 7.1 Hz, 1H), 7.01-6.97 (m, 2H), 5.03 (dd, J=12.8, 5.5 Hz, 1H), 4.74 (s, 2H), 4.13 (s, 3H), 3.77-3.71 (m, 1H), 3.65-3.59 (m, 1H), 3.57-3.51 (m, 1H), 3.32-3.27 (m, 3H), 3.26-3.21 (m, 2H), 2.87-2.79 (m, 2H), 2.76-2.65 (m, 2H), 2.50-2.45 (m, 1H), 2.41 (dd, J=15.0, 7.9 Hz, 1H), 2.35-2.30 (m, 1H), 2.12-2.07 (m, 1H), 1.86-1.78 (m, 1H), 1.69-1.58 (m, 4H). HRMS m/z [M+H]⁺ calcd for C₄₁H₄₅N₁₀O₆⁺ 773.3518, found 773.3555.

### Example 29

### Synthesis of LQ076-73

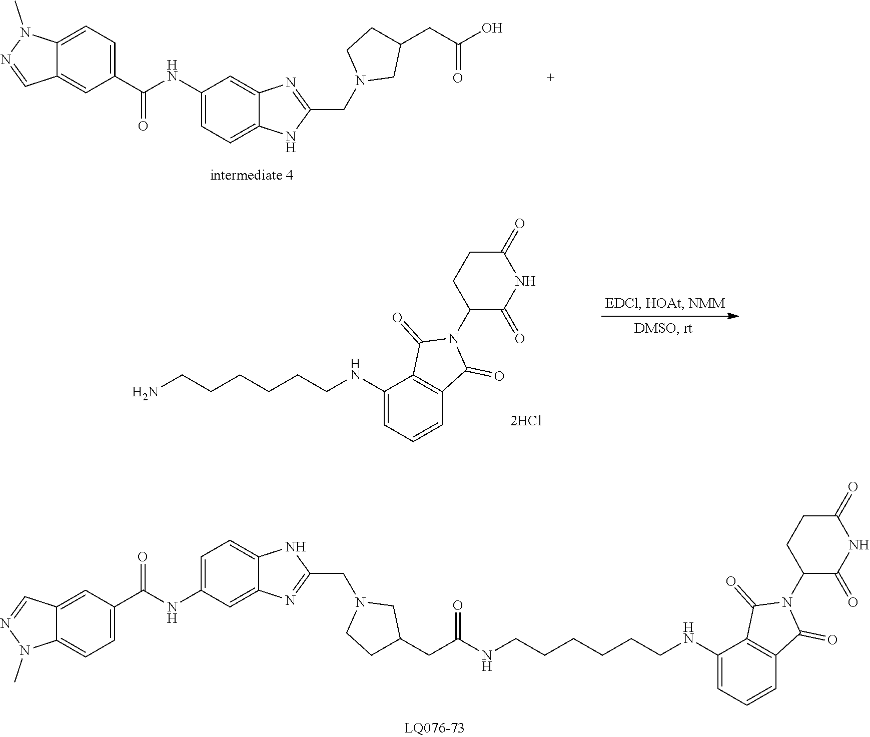 [SMILES: Cn1ncc2cc(C(=O)Nc3ccc4[nH]c(CN5CCC(CC(=O)O)C5)nc4c3)ccc21 ; NCCCCCCNc1cccc2c1C(=O)N(C1CCC(=O)NC1=O)C2=O ; Cn1ncc2cc(C(=O)Nc3ccc4[nH]c(CN5CCC(CC(=O)NCCCCCCNc6cccc7c6C(=O)N(C6CCC(=O)NC6=O)C7=O)C5)nc4c3)ccc21]

LQ076-73 was synthesized following the standard procedure for preparing LQ076-46 from intermediate 4 (12 mg, 0.02 mmol), 4-((6-aminohexyl)amino)-2-(2,6-dioxopiperidin-3-yl)isoindoline-1,3-dione (8.7 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-73 was obtained as yellow solid in TFA salt form (13.8 mg, 68%). ¹H NMR (600 MHz, Methanol-d₄) δ 8.45 (s, 1H), 8.31 (d, J=1.9 Hz, 1H), 8.17 (s, 1H), 8.04 (dd, J=8.8, 1.6 Hz, 1H), 7.69-7.65 (m, 2H), 7.56 (dd, J=8.7, 2.0 Hz, 1H), 7.51 (dd, J=8.5, 7.1 Hz, 1H), 7.01-6.97 (m, 2H), 5.05 (dd, J=12.8, 5.5 Hz, 1H), 4.71 (s, 2H), 4.13 (s, 3H), 3.76-3.71 (m, 1H), 3.65-3.60 (m, 1H), 3.58-3.52 (m, 1H), 3.31-3.26 (m, 3H), 3.21-3.15 (m, 2H), 2.89-2.79 (m, 2H), 2.77-2.67 (m, 2H), 2.48 (dd, J=15.0, 6.0 Hz, 1H), 2.43-2.31 (m, 2H), 2.14-2.08 (m, 1H), 1.87-1.80 (m, 1H), 1.66-1.61 (m, 2H), 1.55-1.49 (m, 2H), 1.46-1.35 (m, 4H). HRMS m/z [M+H]⁺ calcd for C₄₂H₄₇N₁₀O₆⁺ 787.3675, found 787.3702.

### Example 30

### Synthesis of LQ076-74

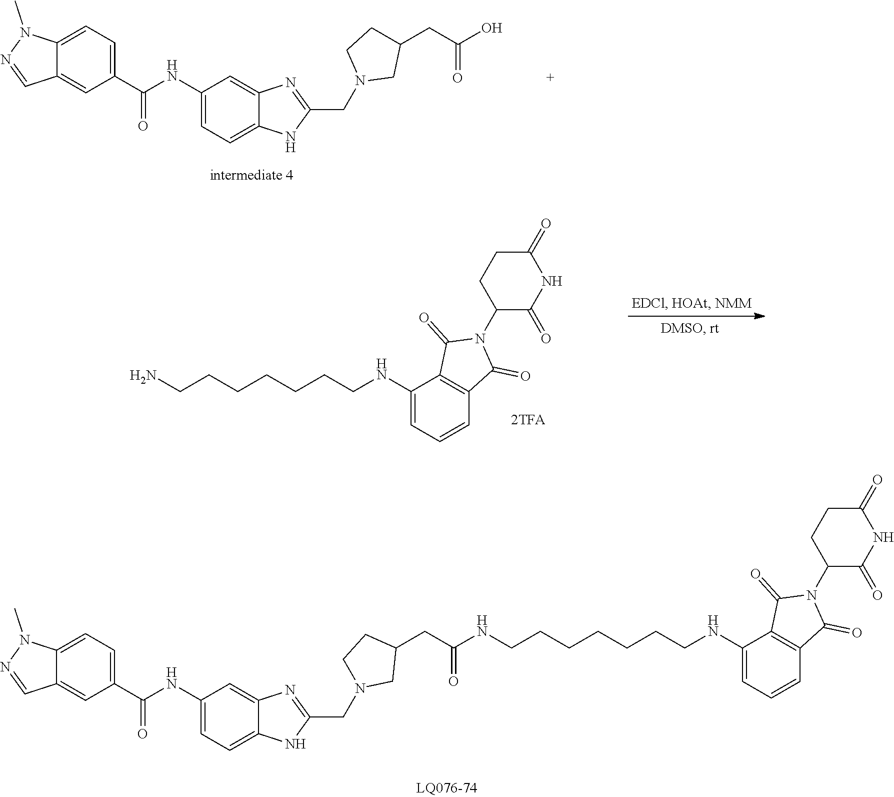 [SMILES: Cn1ncc2cc(C(=O)Nc3ccc4[nH]c(CN5CCC(CC(=O)O)C5)nc4c3)ccc21 ; NCCCCCCCNc1cccc2c1C(=O)N(C1CCC(=O)NC1=O)C2=O ; Cn1ncc2cc(C(=O)Nc3ccc4[nH]c(CN5CCC(CC(=O)NCCCCCCCNc6cccc7c6C(=O)N(C6CCC(=O)NC6=O)C7=O)C5)nc4c3)ccc21]

LQ076-74 was synthesized following the standard procedure for preparing LQ076-46 from intermediate 4 (12 mg, 0.02 mmol), 4-((7-aminoheptyl)amino)-2-(2,6-dioxopiperidin-3-yl)isoindoline-1,3-dione (10.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-74 was obtained as yellow solid in TFA salt form (15.6 mg, 76%). ¹H NMR (600 MHz, Methanol-d₄) δ 8.45 (s, 1H), 8.32 (d, J=1.9 Hz, 1H), 8.17 (s, 1H), 8.03 (dd, J=8.9, 1.7 Hz, 1H), 7.68 (d, J=3.3 Hz, 1H), 7.67 (d, J=3.4 Hz, 1H), 7.58 (dd, J=8.8, 2.0 Hz, 1H), 7.51 (dd, J=8.6, 7.0 Hz, 1H), 7.01-6.97 (m, 2H), 5.05 (dd, J=12.8, 5.5 Hz, 1H), 4.74 (s, 2H), 4.13 (s, 3H), 3.76-3.71 (m, 1H), 3.65-3.60 (m, 1H), 3.58-3.53 (m, 1H), 3.30-3.24 (m, 3H), 3.20-3.13 (m, 2H), 2.89-2.80 (m, 2H), 2.77-2.67 (m, 2H), 2.48 (dd, J=15.0, 6.1 Hz, 1H), 2.40 (dd, J=15.0, 8.0 Hz, 1H), 2.37-2.31 (m, 1H), 2.13-2.08 (m, 1H), 1.88-1.81 (m, 1H), 1.66-1.60 (m, 2H), 1.52-1.47 (m, 2H), 1.43-1.32 (m, 6H). HRMS m/z [M+H]⁺ calcd for C₄₃H₄₉N₁₀O₆⁺ 801.3831, found 801.3872.

### Example 31

### Synthesis of LQ076-75

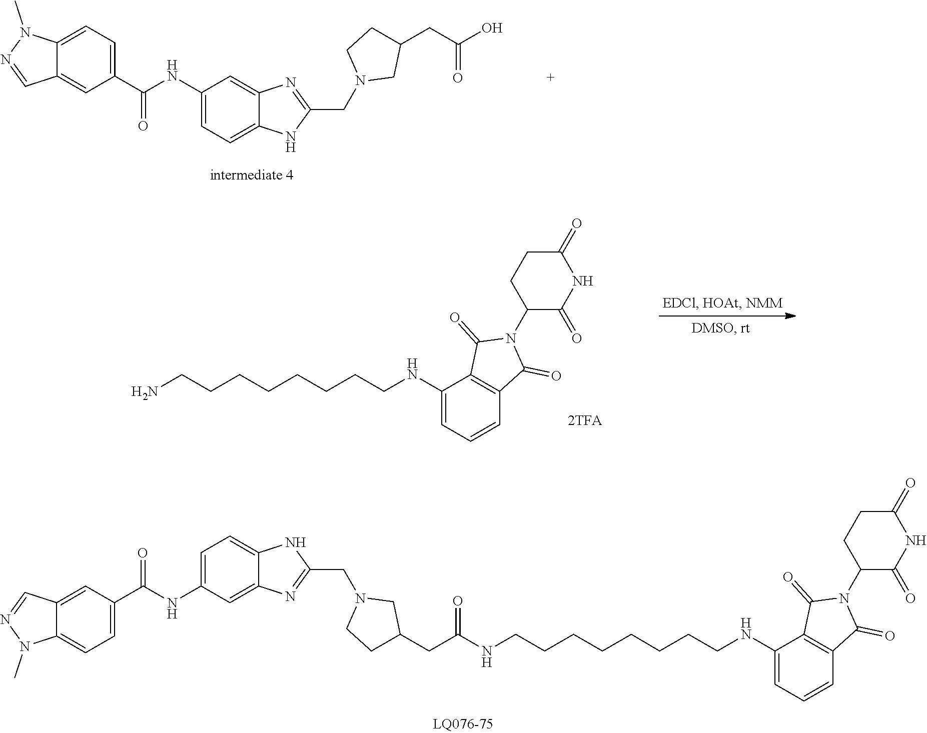 [SMILES: Cn1ncc2cc(C(=O)Nc3ccc4[nH]c(CN5CCC(CC(=O)O)C5)nc4c3)ccc21 ; NCCCCCCCCNc1cccc2c1C(=O)N(C1CCC(=O)NC1=O)C2=O ; Cn1ncc2cc(C(=O)Nc3ccc4[nH]c(CN5CCC(CC(=O)NCCCCCCCCNc6cccc7c6C(=O)N(C6CCC(=O)NC6=O)C7=O)C5)nc4c3)ccc21]

LQ076-75 was synthesized following the standard procedure for preparing LQ076-46 from intermediate 4 (12 mg, 0.02 mmol), 4-((8-aminooctyl)amino)-2-(2,6-dioxopiperidin-3-yl)isoindoline-1,3-dione (11.0 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-75 was obtained as yellow solid in TFA salt form (13.5 mg, 65%). ¹H NMR (600 MHz, Methanol-d₄) δ 8.46 (s, 1H), 8.31 (d, J=2.0 Hz, 1H), 8.17 (s, 1H), 8.04 (dd, J=8.8, 1.7 Hz, 1H), 7.69-7.66 (m, 2H), 7.56 (dd, J=8.7, 1.9 Hz, 1H), 7.52 (dd, J=8.5, 7.1 Hz, 1H), 7.00 (dd, J=11.2, 7.8 Hz, 2H), 5.06 (dd, J=12.7, 5.4 Hz, 1H), 4.71 (s, 2H), 4.13 (s, 3H), 3.76-3.71 (m, 1H), 3.66-3.60 (m, 1H), 3.58-3.53 (m, 1H), 3.30-3.26 (m, 3H), 3.19-3.14 (m, 2H), 2.89-2.79 (m, 2H), 2.77-2.67 (m, 2H), 2.49 (dd, J=15.0, 6.1 Hz, 1H), 2.43-2.33 (m, 2H), 2.14-2.09 (m, 1H), 1.87-1.82 (m, 1H), 1.66-1.61 (m, 2H), 1.52-1.47 (m, 2H), 1.44-1.39 (m, 2H), 1.37-1.32 (m, 6H). HRMS m/z [M+H]⁺ calcd for C₄₄H₅₁N₁₀O₆⁺ 815.3988, found 815.4024.

### Example 32

### Synthesis of Intermediate 7

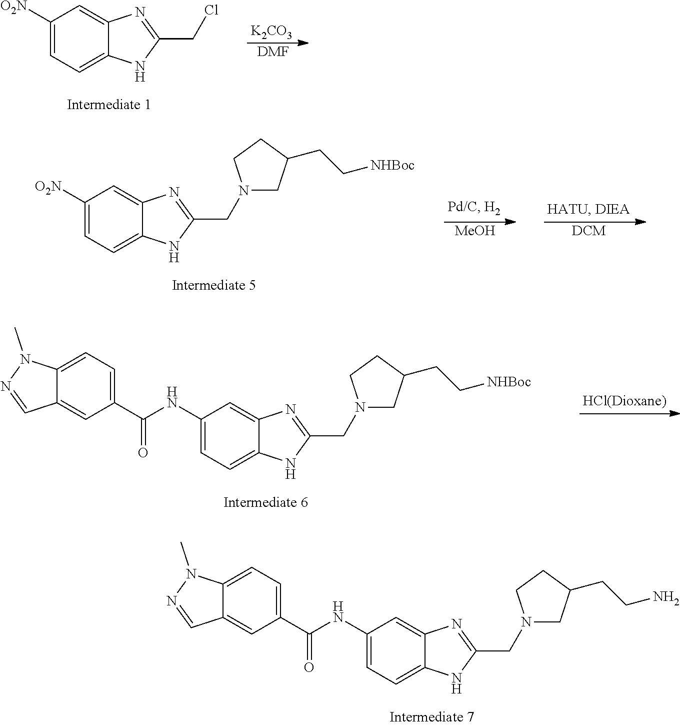

### Intermediate 5: tert-butyl (2-(1-((5-nitro-1H-benzo[d]imidazol-2-yl)methyl)pyrrolidin-3-yl)ethyl)carbamate

Intermediate 5 was synthesized according to the procedures for the preparation of intermediate 2 as a yellow solid in 90% yield. ¹H NMR (600 MHz, Methanol-d₄) δ 8.55 (d, J=2.2 Hz, 1H), 8.21 (dd, J=8.9, 2.2 Hz, 1H), 7.75 (d, J=8.9 Hz, 1H), 4.83 (s, 2H), 3.93-3.56 (m, 3H), 3.17-3.06 (m, 2H), 2.59-2.49 (m, 1H), 2.43-2.33 (m, 1H), 1.89-1.80 (m, 1H), 1.75-1.64 (m, 2H), 1.46-1.38 (m, 10H). MS (ESI): m/z 390.3 [M+H]⁺.

### Intermediate 6: tert-butyl (2-(1-((5-(1-methyl-1H-indazole-5-carboxamido)-1H-benzo[d]imidazol-2-yl)methyl)pyrrolidin-3-yl)ethyl)carbamate

Intermediate 6 was synthesized according to the procedures for the preparation of intermediate 3 as a white solid in 76%. ¹H NMR (600 MHz, Methanol-d₄) δ 8.47 (s, 1H), 8.32 (d, J=2.0 Hz, 1H), 8.17 (d, J=0.9 Hz, 1H), 8.05 (dd, J=8.8, 1.7 Hz, 1H), 7.71-7.66 (m, 2H), 7.61 (dd, J=8.8, 2.0 Hz, 1H), 4.78 (s, 2H), 4.13 (s, 3H), 3.77 (dd, J=11.4, 7.9 Hz, 1H), 3.66-3.57 (m, 2H), 3.22-3.16 (m, 1H), 3.14-3.07 (m, 2H), 2.56-2.47 (m, 1H), 2.40-2.32 (m, 1H), 1.86-1.78 (m, 1H), 1.72-1.65 (m, 2H), 1.43 (s, 9H). MS (ESI): m/z 518.3 [M+H]⁺.

### Intermediate 7: N-(2-((3-(2-aminoethyl)pyrrolidin-1-yl)methyl)-1H-benzo[d]imidazol-5-yl)-1-methyl-1H-indazole-5-carboxamide

Intermediate 6 (100 mg, 0.19 mmol) was dissolved in 1 mL DCM, to the resulting solution was added 1 mL TFA. After being stirred for 1 h at room temperature, the reaction mixture was concentrated and the residue was purified by reverse phase C18 column (10%-100% methanol/0.1% TFA in water) to afford intermediate 7 as white solid in TFA salt form (77 mg, 76%). ¹H NMR (600 MHz, Methanol-d₄) δ 8.46 (dd, J=1.7, 0.8 Hz, 1H), 8.33 (d, J=1.9 Hz, 1H), 8.17 (d, J=0.9 Hz, 1H), 8.04 (dd, J=8.8, 1.7 Hz, 1H), 7.72-7.62 (m, 3H), 4.80 (s, 2H), 4.13 (s, 3H), 3.77 (dd, J=11.3, 7.9 Hz, 1H), 3.67-3.55 (m, 2H), 3.25-3.19 (m, 1H), 3.03-2.96 (m, 2H), 2.60-2.54 (m, 1H), 2.41-2.34 (m, 1H), 1.94-1.80 (m, 3H). MS (ESI): m/z 418.4 [M+H]⁺.

### Example 33

### Synthesis of LQ076-76

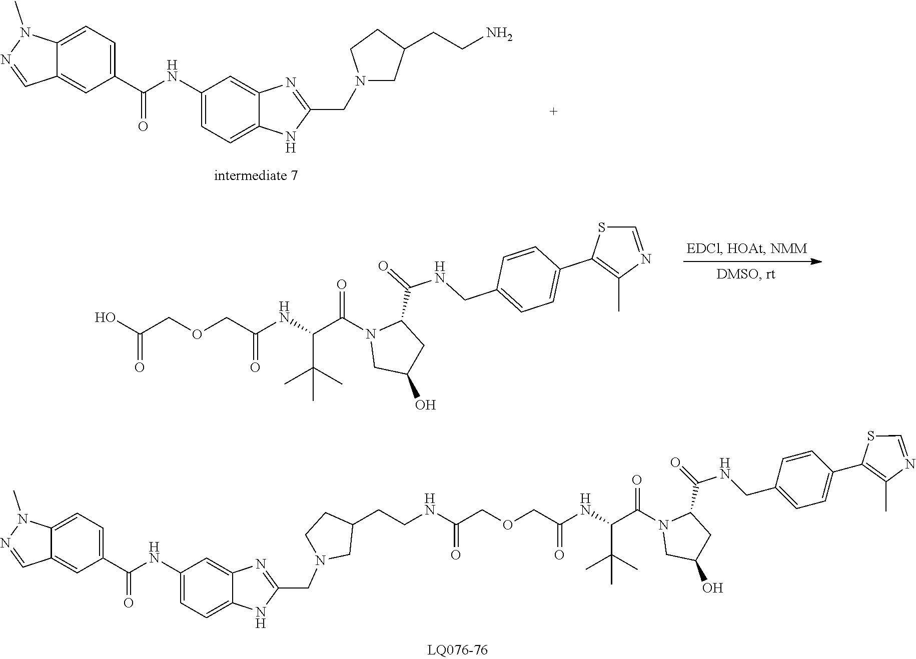 [SMILES: Cn1ncc2cc(C(=O)Nc3ccc4[nH]c(CN5CCC(CCN)C5)nc4c3)ccc21 ; Cc1ncsc1-c1ccc(CNC(=O)[C@@H]2C[C@@H](O)CN2C(=O)[C@@H](NC(=O)COCC(=O)O)C(C)(C)C)cc1 ; Cc1ncsc1-c1ccc(CNC(=O)[C@@H]2C[C@@H](O)CN2C(=O)[C@@H](NC(=O)COCC(=O)NCCC2CCN(Cc3nc4cc(NC(=O)c5ccc6c(cnn6C)c5)ccc4[nH]3)C2)C(C)(C)C)cc1]

To a solution of Intermediate 7 (13 mg, 0.02 mmol) in DMSO (1 mL) were added 2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)acetic acid (10.9 mg, 0.02 mmol, 1.0 equiv), EDCI (1-ethyl-3-(3-dimethylaminopropyl)carbodiimide) (5.8 mg, 0.03 mmol, 1.5 equiv), HOAt (1-hydroxy-7-azabenzo-triazole) (4.1 mg, 0.03 mmol, 1.5 equiv), and NMM (N-Methylmorpholine) (6.1 mg, 0.06 mmol, 3.0 equiv). After being stirred overnight at room temperature, the resulting mixture was purified by preparative HPLC (5%-60% acetonitrile/0.1% TFA in H₂O) to afford LQ076-76 as white solid in TFA salt form (20.6 mg, 88%). ¹H NMR (600 MHz, Methanol-d₄) δ 8.99 (s, 1H), 8.47 (s, 1H), 8.30 (d, J=2.0 Hz, 1H), 8.18 (s, 1H), 8.05 (dd, J=8.8, 1.6 Hz, 1H), 7.71-7.66 (m, 2H), 7.57 (dd, J=8.7, 2.0 Hz, 1H), 7.48-7.44 (m, 2H), 7.43-7.40 (m, 2H), 4.75-4.71 (m, 3H), 4.61-4.57 (m, 1H), 4.54-4.49 (m, 2H), 4.38 (d, J=15.5 Hz, 1H), 4.15-4.05 (m, 6H), 3.93-3.89 (m, 1H), 3.83 (dd, J=11.0, 3.7 Hz, 1H), 3.80-3.75 (m, 1H), 3.66-3.56 (m, 2H), 3.23-3.17 (m, 1H), 2.54-2.46 (m, 4H), 2.39-2.33 (m, 1H), 2.29-2.23 (m, 1H), 2.13-2.08 (m, 1H), 1.84-1.73 (m, 3H), 1.06 (s, 9H). HRMS m/z [M+H]⁺ calcd for C₄₉H₆₀N₁₁O₇S⁺ 946.4392, found 946.4428.

### Example 34

### Synthesis of LQ076-77

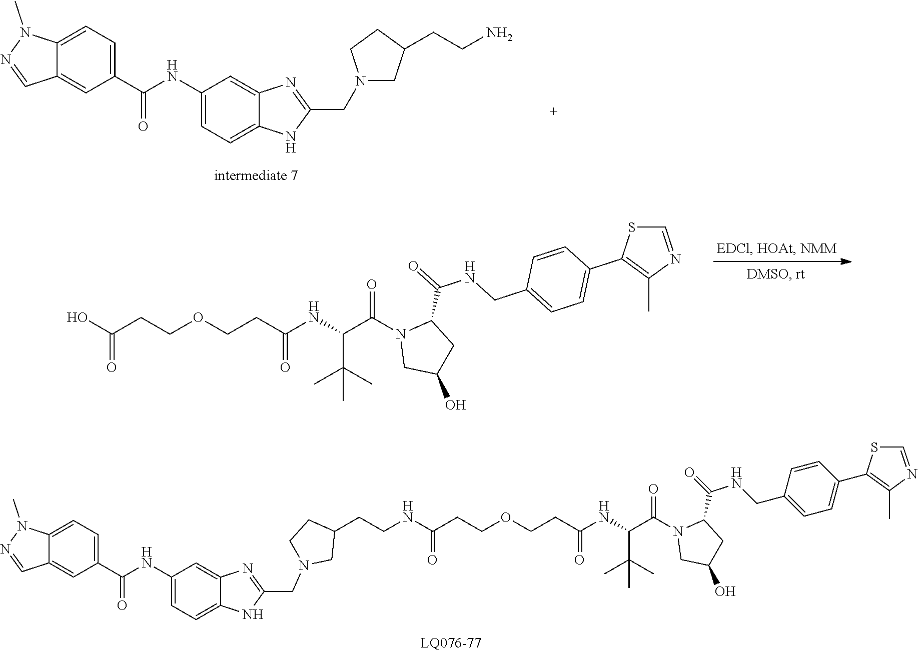 [SMILES: Cn1ncc2cc(C(=O)Nc3ccc4[nH]c(CN5CCC(CCN)C5)nc4c3)ccc21 ; Cc1ncsc1-c1ccc(CNC(=O)[C@@H]2C[C@@H](O)CN2C(=O)[C@@H](NC(=O)CCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCC(=O)NCCC2CCN(Cc3nc4cc(NC(=O)c5ccc6c(cnn6C)c5)ccc4[nH]3)C2)C(C)(C)C)cc1]

LQ076-77 was synthesized following the standard procedure for preparing LQ076-76 from intermediate 7 (13 mg, 0.02 mmol), 3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoic acid (11.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-77 was obtained as white solid in TFA salt form (20.4 mg, 85%). ¹H NMR (600 MHz, Methanol-d₄) δ 9.03 (s, 1H), 8.46 (s, 1H), 8.34-8.32 (m, 1H), 8.18 (s, 1H), 8.04 (d, J=8.8 Hz, 1H), 7.70-7.67 (m, 2H), 7.59 (dd, J=8.8, 2.0 Hz, 1H), 7.47-7.44 (m, 2H), 7.43-7.39 (m, 2H), 4.76 (s, 2H), 4.65-4.63 (m, 1H), 4.61-4.56 (m, 1H), 4.54-4.49 (m, 2H), 4.38 (d, J=15.5 Hz, 1H), 4.14 (s, 3H), 3.91-3.86 (m, 1H), 3.82-3.67 (m, 5H), 3.64-3.58 (m, 2H), 3.28-3.16 (m, 3H), 2.58-2.42 (m, 9H), 2.38-2.32 (m, 1H), 2.27-2.22 (m, 1H), 2.12-2.06 (m, 1H), 1.85-1.79 (m, 1H), 1.73-1.68 (m, 2H), 1.05 (s, 9H). HRMS m/z [M+H]⁺ calcd for C₅₁H₆₄N₁₁O₇S⁺ 946.4705, found 974.4784.

### Example 35

### Synthesis of LQ076-78

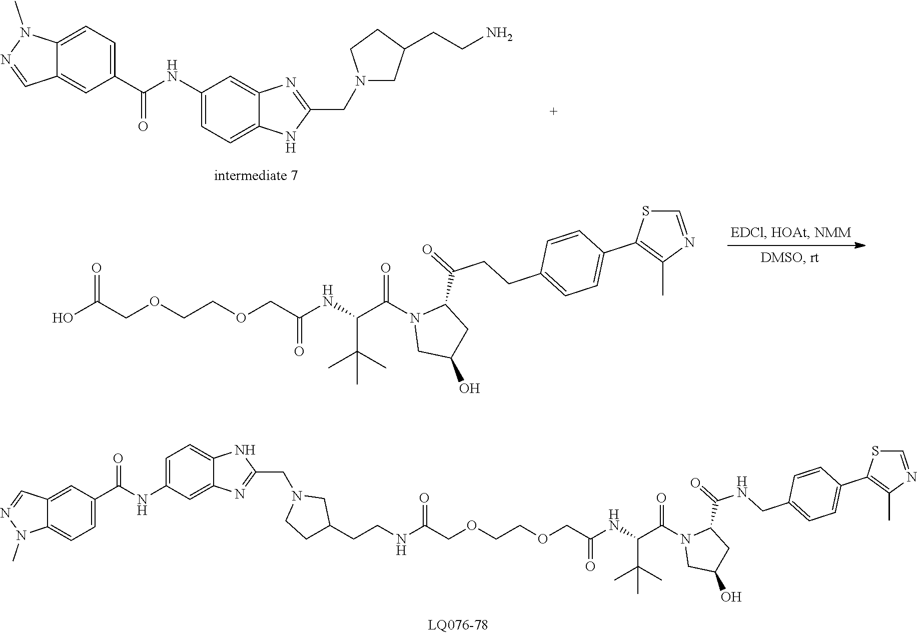 [SMILES: Cn1ncc2cc(C(=O)Nc3ccc4[nH]c(CN5CCC(CCN)C5)nc4c3)ccc21 ; Cc1ncsc1-c1ccc(CCC(=O)[C@@H]2C[C@@H](O)CN2C(=O)[C@@H](NC(=O)COCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCC(=O)NCCC2CCN(Cc3nc4cc(NC(=O)c5ccc6c(cnn6C)c5)ccc4[nH]3)C2)C(C)(C)C)cc1]

LQ076-78 was synthesized following the standard procedure for preparing LQ076-76 from intermediate 7 (13 mg, 0.02 mmol), 2-(2-(2-(((S)-1-((2S,4R)-4-hydroxy-2-(3-(4-(4-methylthiazol-5-yl)phenyl)propanoyl)pyrrolidin-1-yl)-3,3-dimethyl-1-oxobutan-2-yl)amino)-2-oxoethoxy)ethoxy)acetic acid (11.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-78 was obtained as white solid in TFA salt form (19.5 mg, 80%). ¹H NMR (600 MHz, Methanol-d₄) δ 9.03 (s, 1H), 8.46 (s, 1H), 8.33 (s, 1H), 8.18 (s, 1H), 8.04 (dd, J=8.8, 1.6 Hz, 1H), 7.70-7.66 (m, 2H), 7.59 (dd, J=8.8, 2.0 Hz, 1H), 7.47-7.40 (m, 4H), 4.75 (s, 2H), 4.72-4.69 (m, 1H), 4.61-4.56 (m, 2H), 4.52-4.48 (m, 1H), 4.47-4.42 (m, 1H), 4.15-4.00 (m, 6H), 3.88 (d, J=11.1 Hz, 1H), 3.83-3.69 (m, 6H), 3.65-3.56 (m, 2H), 3.32-3.27 (m, 2H), 3.22-3.15 (m, 1H), 2.52-2.44 (m, 5H), 2.38-2.31 (m, 1H), 2.29-2.23 (m, 1H), 2.11-2.05 (m, 1H), 1.84-1.69 (m, 3H), 1.05 (s, 9H). HRMS m/z [M+H]⁺ calcd for C₅₁H₆₄N₁₁O₈S⁺ 990.4655, found 990.4723.

### Example 36

### Synthesis of LQ076-79

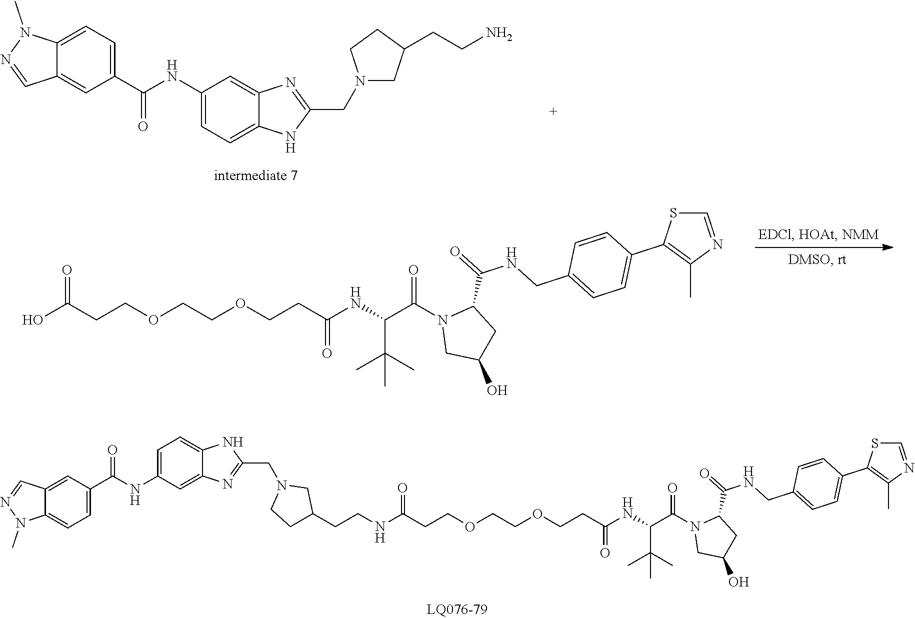 [SMILES: Cn1ncc2cc(C(=O)Nc3ccc4[nH]c(CN5CCC(CCN)C5)nc4c3)ccc21 ; Cc1ncsc1-c1ccc(CNC(=O)[C@@H]2C[C@@H](O)CN2C(=O)[C@@H](NC(=O)CCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCC(=O)NCCC2CCN(Cc3nc4cc(NC(=O)c5ccc6c(cnn6C)c5)ccc4[nH]3)C2)C(C)(C)C)cc1]

LQ076-79 was synthesized following the standard procedure for preparing LQ076-76 from intermediate 7 (13 mg, 0.02 mmol), 3-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)propanoic acid (12.4 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-79 was obtained as white solid in TFA salt form (19.4 mg, 78%). ¹H NMR (600 MHz, Methanol-d₄) δ 9.07 (s, 1H), 8.46 (s, 1H), 8.35 (d, J=2.0 Hz, 1H), 8.18 (s, 1H), 8.04 (dd, J=8.8, 1.7 Hz, 1H), 7.72-7.67 (m, 2H), 7.62 (dd, J=8.8, 2.0 Hz, 1H), 7.49-7.40 (m, 4H), 4.79 (s, 2H), 4.66-4.64 (m, 1H), 4.62-4.57 (m, 1H), 4.55-4.49 (m, 2H), 4.40-4.35 (m, 1H), 4.13 (s, 3H), 3.90 (d, J=11.0 Hz, 1H), 3.82-3.69 (m, 6H), 3.67-3.56 (m, 6H), 3.30-3.18 (m, 3H), 2.60-2.40 (m, 8H), 2.38-2.31 (m, 1H), 2.27-2.21 (m, 1H), 2.12-2.06 (m, 1H), 1.86-1.78 (m, 1H), 1.73-1.68 (m, 2H), 1.05 (s, 9H). HRMS m/z [M+H]⁺ calcd for C₅₃H₆₈N₁₁O₈S⁺ 1018.4968, found 1018.5060.

### Example 37

### Synthesis of LQ076-80

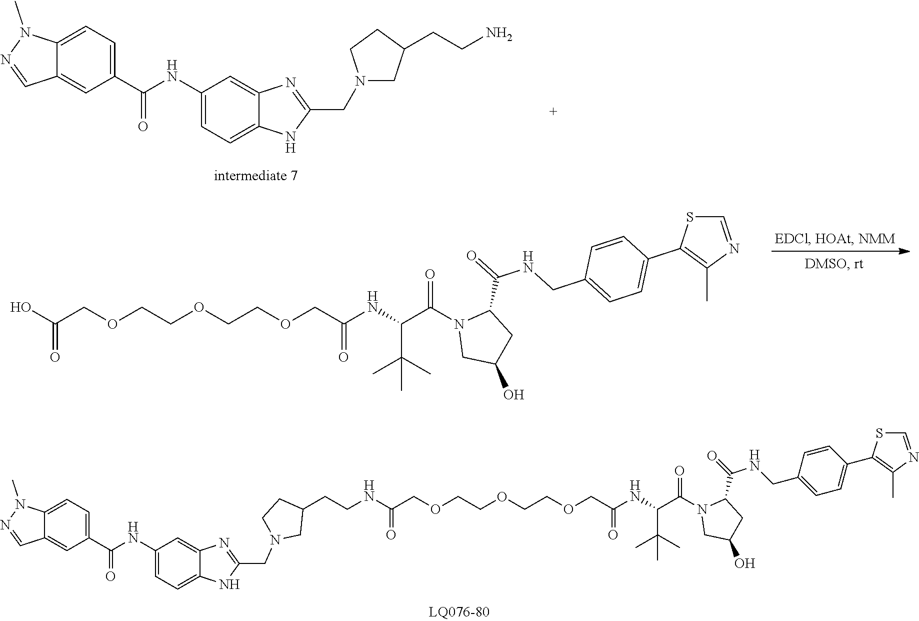 [SMILES: Cn1ncc2cc(C(=O)Nc3ccc4[nH]c(CN5CCC(CCN)C5)nc4c3)ccc21 ; Cc1ncsc1-c1ccc(CNC(=O)[C@@H]2C[C@@H](O)CN2C(=O)[C@@H](NC(=O)COCCOCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCCOCC(=O)NCCC2CCN(Cc3nc4cc(NC(=O)c5ccc6c(cnn6C)c5)ccc4[nH]3)C2)C(C)(C)C)cc1]

LQ076-80 was synthesized following the standard procedure for preparing LQ076-76 from intermediate 7 (13 mg, 0.02 mmol), (S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecanoic acid (12.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-80 was obtained as white solid in TFA salt form (18 mg, 71%). ¹H NMR (800 MHz, Methanol-d₄) δ 8.97 (s, 1H), 8.47 (s, 1H), 8.32 (s, 1H), 8.19 (s, 1H), 8.05 (d, J=8.8 Hz, 1H), 7.71-7.67 (m, 2H), 7.57 (d, J=8.7 Hz, 1H), 7.48-7.41 (m, 4H), 4.74-4.68 (m, 3H), 4.60-4.50 (m, 3H), 4.37 (d, J=15.3 Hz, 1H), 4.15 (s, 3H), 4.11-4.03 (m, 2H), 4.02-3.92 (m, 2H), 3.88 (d, J=11.1 Hz, 1H), 3.82-3.57 (m, 12H), 3.32-3.26 (m, 2H), 3.22-3.16 (m, 1H), 2.50-2.45 (m, 4H), 2.38-2.33 (m, 1H), 2.25 (dd, J=13.2, 7.6 Hz, 1H), 2.13-2.08 (m, 1H), 1.84-1.79 (m, 1H), 1.75-1.71 (m, 2H), 1.06 (s, 9H). HRMS m/z [M+H]⁺ calcd for C₅₃H₆₈N₁₁O₉S⁺ 1034.4917, found 1034.4890.

### Example 38

### Synthesis of LQ076-81

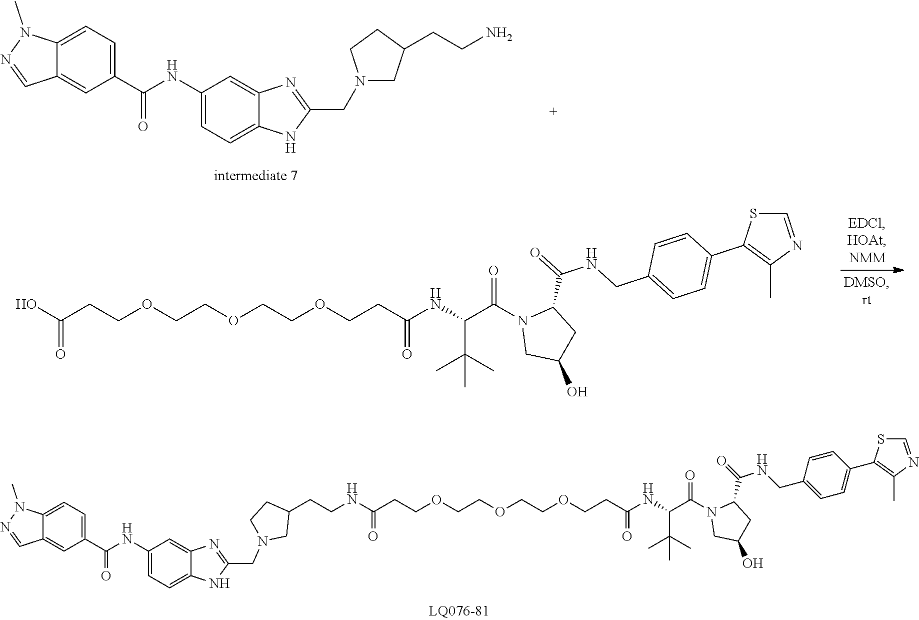 [SMILES: Cn1ncc2cc(C(=O)Nc3ccc4[nH]c(CN5CCC(CCN)C5)nc4c3)ccc21 ; Cc1ncsc1-c1ccc(CNC(=O)[C@@H]2C[C@@H](O)CN2C(=O)[C@@H](NC(=O)CCOCCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCOCCC(=O)NCCC2CCN(Cc3nc4cc(NC(=O)c5ccc6c(cnn6C)c5)ccc4[nH]3)C2)C(C)(C)C)cc1]

LQ076-81 was synthesized following the standard procedure for preparing LQ076-76 from intermediate 7 (13 mg, 0.02 mmol), (S)-15-((2S,4R)-4-hydroxy-2-((4-(4-methylthiazol-5-yl)benzyl)carbamoyl)pyrrolidine-1-carbonyl)-16,16-dimethyl-13-oxo-4,7,10-trioxa-14-azaheptadecanoic acid (13.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-81 was obtained as white solid in TFA salt form (18.4 mg, 72%). ¹H NMR (600 MHz, Methanol-d₄) δ 9.06 (s, 1H), 8.47 (s, 1H), 8.34 (d, J=1.9 Hz, 1H), 8.18 (s, 1H), 8.05 (dd, J=8.9, 1.7 Hz, 1H), 7.71-7.67 (m, 2H), 7.61 (dd, J=8.8, 2.0 Hz, 1H), 7.49-7.40 (m, 4H), 4.79 (s, 2H), 4.66-4.64 (m, 1H), 4.61-4.56 (m, 1H), 4.55-4.49 (m, 2H), 4.37 (d, J=15.5 Hz, 1H), 4.13 (s, 3H), 3.90 (d, J=11.0 Hz, 1H), 3.82-3.69 (m, 6H), 3.66-3.56 (m, 11H), 3.29-3.17 (m, 3H), 2.60-2.55 (m, 1H), 2.53-2.42 (m, 6H), 2.38-2.32 (m, 1H), 2.24 (dd, J=13.3, 7.7 Hz, 1H), 2.12-2.06 (m, 1H), 1.85-1.78 (m, 1H), 1.73-1.68 (m, 2H), 1.05 (s, 9H). HRMS m/z [M+H]+ calcd for C₅₅H₇₂N₁₁O₉S⁺ 1062.5230, found 1062.5310.

### Example 39

### Synthesis of LQ076-82

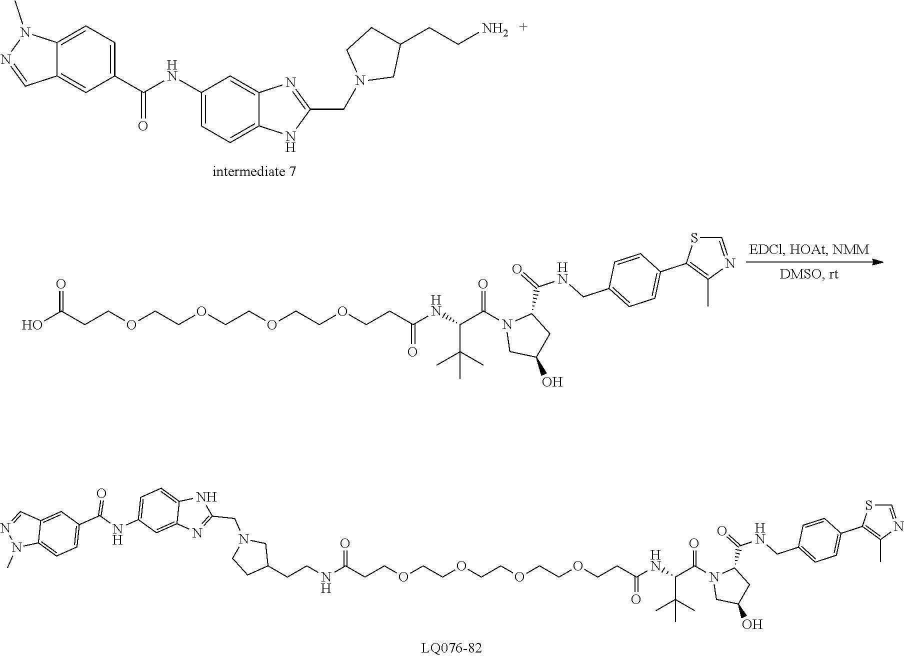 [SMILES: Cn1ncc2cc(C(=O)Nc3ccc4[nH]c(CN5CCC(CCN)C5)nc4c3)ccc21 ; "+" ; Cc1ncsc1-c1ccc(CNC(=O)[C@@H]2C[C@@H](O)CN2C(=O)[C@@H](NC(=O)CCOCCOCCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCOCCOCCC(=O)NCCC2CCN(Cc3nc4cc(NC(=O)c5ccc6c(cnn6C)c5)ccc4[nH]3)C2)C(C)(C)C)cc1]

LQ076-82 was synthesized following the standard procedure for preparing LQ076-76 from intermediate 7 (13 mg, 0.02 mmol), (S)-18-((2S,4R)-4-hydroxy-2-((4-(4-methylthiazol-5-yl)benzyl)carbamoyl)pyrrolidine-1-carbonyl)-19,19-dimethyl-16-oxo-4,7,10,13-tetraoxa-17-azaicosanoic acid (14.6 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL).

LQ076-82 was obtained as white solid in TFA salt form (17.2 mg, 65%). ¹H NMR (600 MHz, Methanol-d₄) δ 9.10 (s, 1H), 8.47 (s, 1H), 8.35 (s, 1H), 8.19 (s, 1H), 8.05 (d, J=8.8 Hz, 1H), 7.72-7.68 (m, 2H), 7.60 (dd, J=8.7, 2.0 Hz, 1H), 7.50-7.41 (m, 4H), 4.77 (s, 2H), 4.66-4.64 (m, 1H), 4.60-4.49 (m, 3H), 4.38 (d, J=15.5 Hz, 1H), 4.14 (s, 3H), 3.90 (d, J=11.0 Hz, 1H), 3.83-3.69 (m, 6H), 3.66-3.57 (m, 15H), 3.29-3.17 (m, 3H), 2.60-2.55 (m, 1H), 2.52-2.42 (m, 5H), 2.39-2.33 (m, 1H), 2.26-2.21 (m, 1H), 2.12-2.06 (m, 1H), 1.86-1.79 (m, 1H), 1.74-1.69 (m, 2H), 1.05 (s, 9H). HRMS m/z [M+H]⁺ calcd for C₅₇H₇₆N₁₁O₁₀S⁺ 1106.5492, found 1106.5516.

### Example 40

### Synthesis of LQ076-83

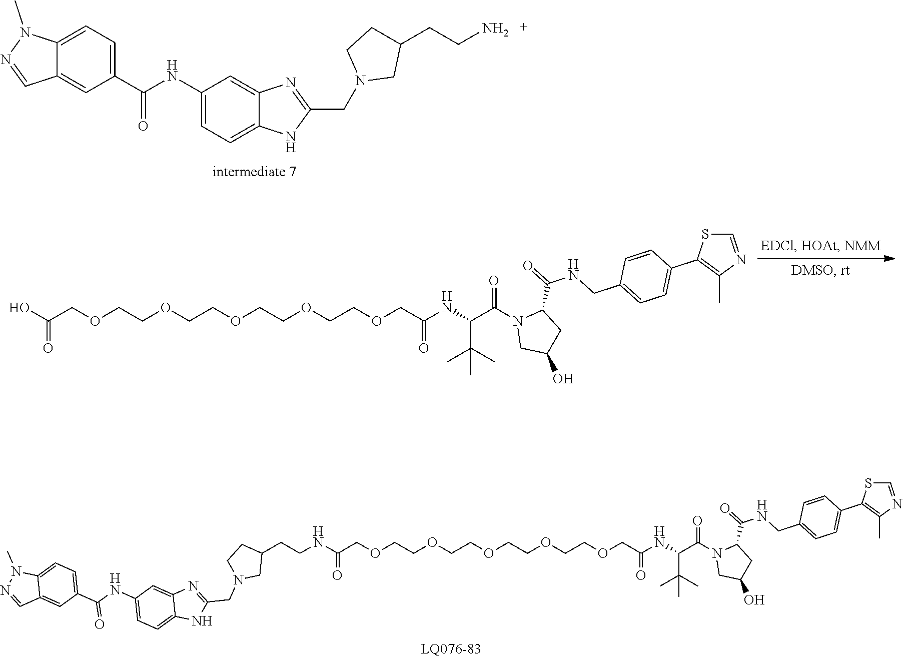 [SMILES: Cn1ncc2cc(C(=O)Nc3ccc4[nH]c(CN5CCC(CCN)C5)nc4c3)ccc21 ; "+" ; Cc1ncsc1-c1ccc(CNC(=O)[C@@H]2C[C@@H](O)CN2C(=O)[C@@H](NC(=O)COCCOCCOCCOCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCCOCCOCCOCC(=O)NCCC2CCN(Cc3nc4cc(NC(=O)c5ccc6c(cnn6C)c5)ccc4[nH]3)C2)C(C)(C)C)cc1]

LQ076-83 was synthesized following the standard procedure for preparing LQ076-76 from intermediate 7 (13 mg, 0.02 mmol), (S)-19-((2S,4R)-4-hydroxy-2-((4-(4-methylthiazol-5-yl)benzyl)carbamoyl)pyrrolidine-1-carbonyl)-20,20-dimethyl-17-oxo-3,6,9,12,15-pentaoxa-18-azahenicosanoic acid (15.0 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-83 was obtained as white solid in TFA salt form (18.9 mg, 70%). ¹H NMR (600 MHz, Methanol-d₄) δ 9.01 (s, 1H), 8.46 (s, 1H), 8.33 (s, 1H), 8.18 (s, 1H), 8.04 (dd, J=8.8, 1.7 Hz, 1H), 7.70-7.67 (m, 2H), 7.59 (dd, J=8.7, 2.0 Hz, 1H), 7.48-7.40 (m, 4H), 4.75 (s, 2H), 4.69-4.67 (m, 1H), 4.62-4.57 (m, 1H), 4.56-4.49 (m, 2H), 4.37 (d, J=15.5 Hz, 1H), 4.14 (s, 3H), 4.06-4.03 (m, 2H), 3.99-3.96 (m, 2H), 3.91-3.87 (m, 1H), 3.83-3.76 (m, 2H), 3.71-3.58 (m, 18H), 3.32-3.27 (m, 2H), 3.23-3.17 (m, 1H), 2.52-2.45 (m, 4H), 2.40-2.32 (m, 1H), 2.27-2.22 (m, 1H), 2.12-2.07 (m, 1H), 1.86-1.80 (m, 1H), 1.77-1.72 (m, 2H), 1.06 (s, 9H). HRMS m/z [M+H]⁺ calcd for C₅₇H₇₆N₁₁O₁₁S⁺ 1122.5441, found 1122.5517.

### Example 41

### Synthesis of LQ076-84

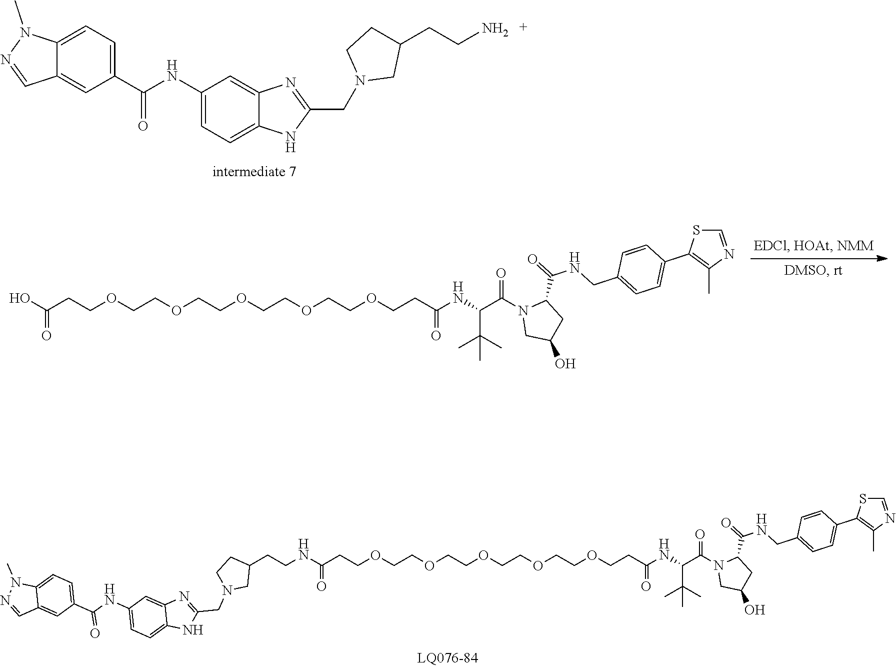 [SMILES: Cn1ncc2cc(C(=O)Nc3ccc4[nH]c(CN5CCC(CCN)C5)nc4c3)ccc21 ; "+" ; Cc1ncsc1-c1ccc(CNC(=O)[C@@H]2C[C@@H](O)CN2C(=O)[C@@H](NC(=O)CCOCCOCCOCCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCOCCOCCOCCC(=O)NCCC2CCN(Cc3nc4cc(NC(=O)c5ccc6c(cnn6C)c5)ccc4[nH]3)C2)C(C)(C)C)cc1]

LQ076-84 was synthesized following the standard procedure for preparing LQ076-76 from intermediate 7 (13 mg, 0.02 mmol), (S)-21-((2S,4R)-4-hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azatricosanoic acid (15.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-84 was obtained as white solid in TFA salt form (20 mg, 73%). ¹H NMR (600 MHz, Methanol-d₄) δ 9.02 (s, 1H), 8.47 (s, 1H), 8.34-8.31 (m, 1H), 8.19 (s, 1H), 8.05 (dd, J=8.8, 1.7 Hz, 1H), 7.71-7.67 (m, 2H), 7.60-7.57 (m, 1H), 7.49-7.41 (m, 4H), 4.75 (s, 2H), 4.66-4.64 (m, 1H), 4.61-4.49 (m, 3H), 4.37 (d, J=15.5 Hz, 1H), 4.14 (s, 3H), 3.90 (d, J=11.0 Hz, 1H), 3.82-3.56 (m, 25H), 3.30-3.17 (m, 3H), 2.60-2.41 (m, 7H), 2.39-2.32 (m, 1H), 2.26-2.21 (m, 1H), 2.12-2.06 (m, 1H), 1.85-1.78 (m, 1H), 1.74-1.69 (m, 2H), 1.05 (s, 9H). HRMS m/z [M+H]⁺ calcd for C₅₉H₈₀N₁₁O₁₁S⁺ 1150.5754, found 1150.5834.

### Example 42

### Synthesis of LQ076-85

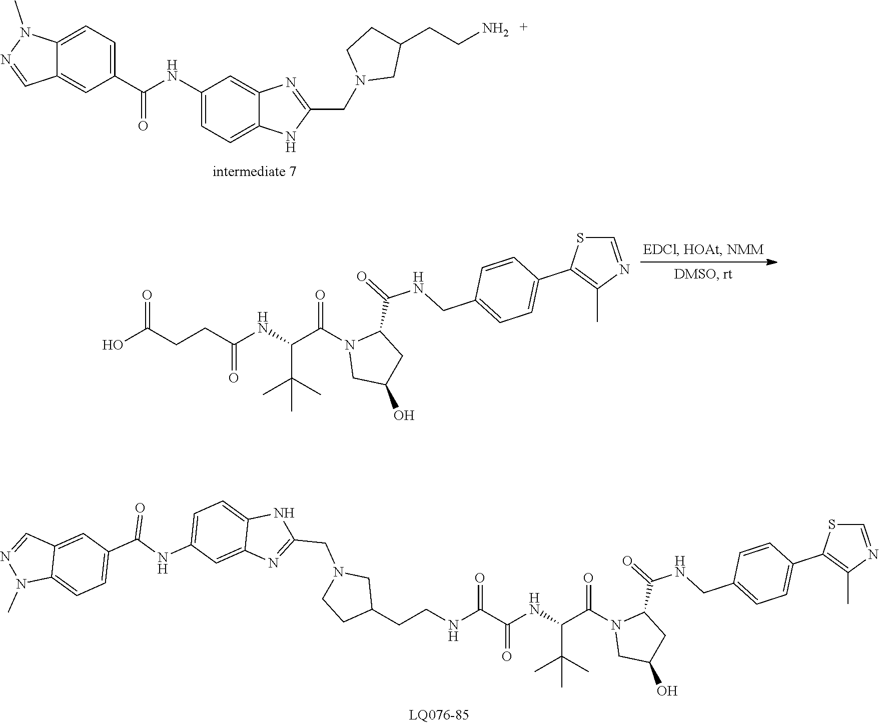

LQ076-85 was synthesized following the standard procedure for preparing LQ076-76 from intermediate 7 (13 mg, 0.02 mmol), 4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-4-oxobutanoic acid (10.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-85 was obtained as white solid in TFA salt form (17.8 mg, 77%). ¹H NMR (600 MHz, Methanol-d₄) δ 9.07 (s, 1H), 8.47 (s, 1H), 8.33 (d, J=2.0 Hz, 1H), 8.18 (s, 1H), 8.05 (d, J=8.8 Hz, 1H), 7.71-7.67 (m, 2H), 7.60 (dd, J=8.8, 2.0 Hz, 1H), 7.48-7.40 (m, 4H), 4.76 (s, 2H), 4.61-4.47 (m, 4H), 4.40-4.35 (m, 1H), 4.14 (s, 3H), 3.93-3.88 (m, 1H), 3.83-3.75 (m, 2H), 3.66-3.56 (m, 2H), 3.30-3.17 (m, 3H), 2.66-2.46 (m, 8H), 2.35 (d, 1H), 2.26-2.20 (m, 1H), 2.12-2.06 (m, 1H), 1.85-1.77 (m, 1H), 1.73-1.67 (m, 2H), 1.08-1.03 (m, 9H). HRMS m/z [M+H]⁺ calcd for C₄₉H₆₀N₁₁O₆S⁺ 930.4443, found 930.4498.

### Example 43

### Synthesis of LQ076-86

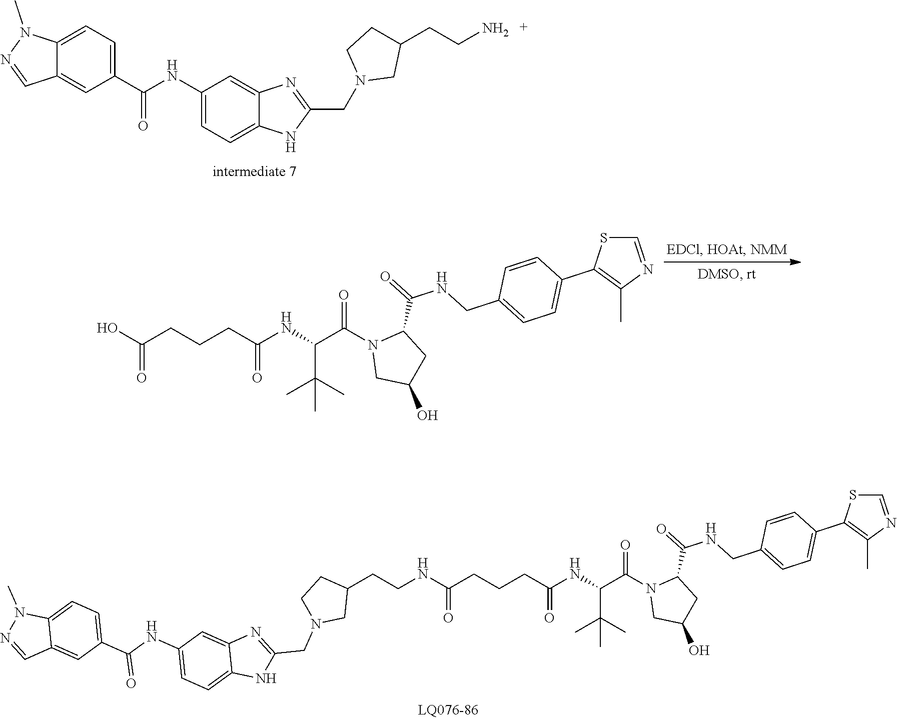

LQ076-86 was synthesized following the standard procedure for preparing LQ076-76 from intermediate 7 (13 mg, 0.02 mmol), 5-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-5-oxopentanoic acid (11.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-86 was obtained as white solid in TFA salt form (18.6 mg, 79%). ¹H NMR (600 MHz, Methanol-d₄) δ 9.00 (s, 1H), 8.47 (s, 1H), 8.31 (s, 1H), 8.18 (s, 1H), 8.05 (dd, J=8.8, 1.6 Hz, 1H), 7.71-7.66 (m, 2H), 7.58 (dd, J=8.7, 2.0 Hz, 1H), 7.48-7.39 (m, 4H), 4.74 (s, 2H), 4.64-4.56 (m, 2H), 4.54-4.49 (m, 2H), 4.38 (d, J=15.7 Hz, 1H), 4.14 (s, 3H), 3.94 (d, J=10.9 Hz, 1H), 3.84-3.74 (m, 2H), 3.68-3.56 (m, 2H), 3.29-3.15 (m, 3H), 2.50-2.46 (m, 4H), 2.38-2.19 (m, 6H), 2.12-2.07 (m, 1H), 1.94-1.86 (m, 2H), 1.84-1.77 (m, 1H), 1.74-1.66 (m, 2H), 1.05 (s, 9H). HRMS m/z [M+H]⁺ calcd for C₅₀H₆₂N₁₁O₆S⁺ 944.4600, found 944.4639.

### Example 44

### Synthesis of LQ076-87

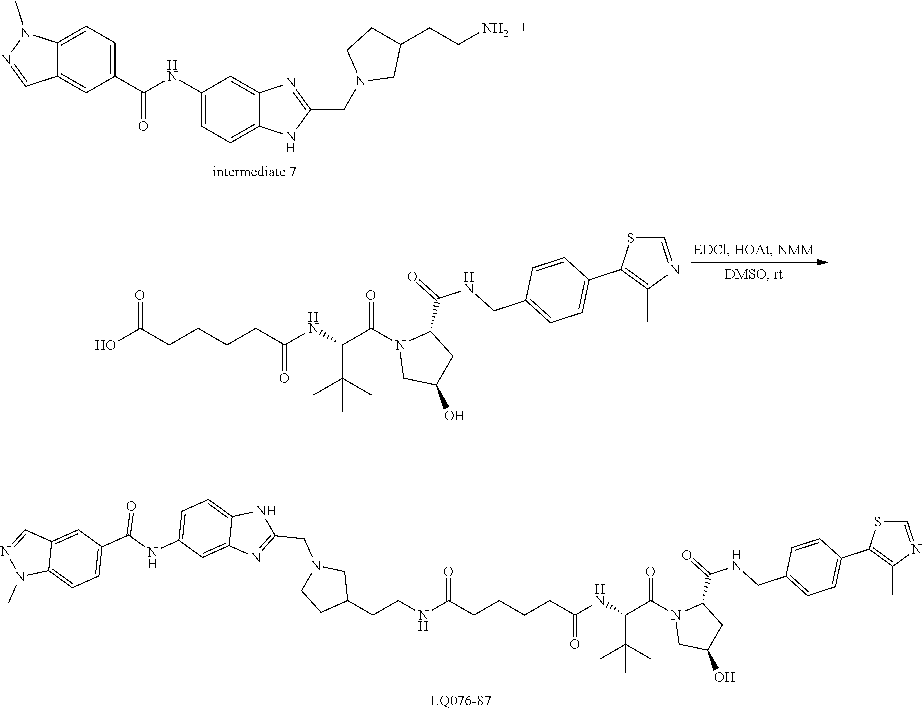

LQ076-87 was synthesized following the standard procedure for preparing LQ076-76 from intermediate 7 (13 mg, 0.02 mmol), 6-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-6-oxohexanoic acid (11.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-87 was obtained as white solid in TFA salt form (19.4 mg, 82%). ¹H NMR (600 MHz, Methanol-d₄) δ 9.11 (s, 1H), 8.46 (s, 1H), 8.36 (s, 1H), 8.17 (s, 1H), 8.06-8.03 (m, 1H), 7.72-7.66 (m, 2H), 7.63 (dd, J=8.8, 2.0 Hz, 1H), 7.49-7.40 (m, 4H), 4.81 (s, 2H), 4.63-4.48 (m, 4H), 4.37 (d, J=15.5 Hz, 1H), 4.13 (s, 3H), 3.90 (dd, J=11.1, 4.7 Hz, 1H), 3.81-3.75 (m, 2H), 3.67-3.59 (m, 2H), 3.29-3.18 (m, 3H), 2.54-2.47 (m, 4H), 2.39-2.16 (m, 7H), 2.12-2.06 (m, 1H), 1.86-1.79 (m, 1H), 1.74-1.56 (m, 6H), 1.08-1.02 (m, 9H). HRMS m/z [M+H]⁺ calcd for C₅₁H₆₄N₁₁O₆S⁺ 958.4756, found 958.4833.

### Example 45

### Synthesis of LQ076-88

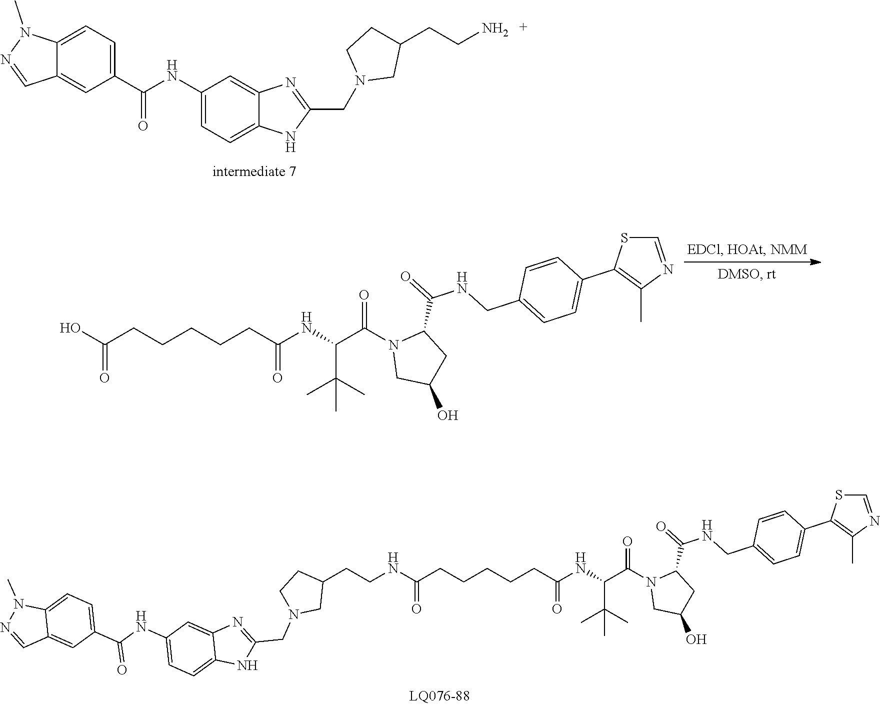

LQ076-88 was synthesized following the standard procedure for preparing LQ076-76 from intermediate 7 (13 mg, 0.02 mmol), 7-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-7-oxoheptanoic acid (11.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-88 was obtained as white solid in TFA salt form (19.8 mg, 82%). ¹H NMR (600 MHz, Methanol-d₄) δ 9.04 (s, 1H), 8.47 (s, 1H), 8.33 (d, J=2.0 Hz, 1H), 8.18 (s, 1H), 8.05 (dd, J=8.8, 1.7 Hz, 1H), 7.70-7.67 (m, 2H), 7.59 (dd, J=8.7, 2.0 Hz, 1H), 7.49-7.40 (m, 4H), 4.76 (s, 2H), 4.65-4.62 (m, 1H), 4.61-4.49 (m, 3H), 4.37 (d, J=15.5 Hz, 1H), 4.14 (s, 3H), 3.90 (d, J=11.0 Hz, 1H), 3.83-3.74 (m, 2H), 3.66-3.58 (m, 2H), 3.27-3.17 (m, 3H), 2.52-2.45 (m, 4H), 2.39-2.21 (m, 4H), 2.20-2.15 (m, 2H), 2.12-2.07 (m, 1H), 1.86-1.78 (m, 1H), 1.74-1.67 (m, 2H), 1.65-1.58 (m, 4H), 1.37-1.32 (m, 2H), 1.04 (s, 9H). HRMS m/z [M+H]⁺ calcd for C₅₂H₆₆N₁₁O₆S⁺ 972.4913, found 972.4950.

### Example 46

### Synthesis of LQ076-89

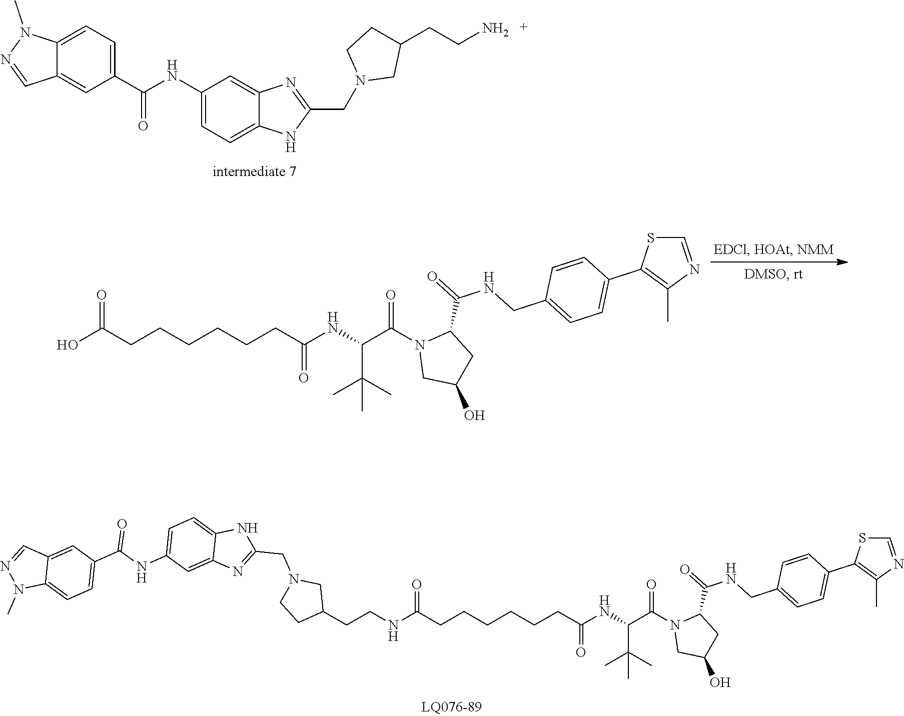

LQ076-89 was synthesized following the standard procedure for preparing LQ076-76 from intermediate 7 (13 mg, 0.02 mmol), 8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctanoic acid (12.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-89 was obtained as white solid in TFA salt form (21.1 mg, 87%). ¹H NMR (600 MHz, Methanol-d₄) δ 9.07 (s, 1H), 8.47 (s, 1H), 8.35-8.33 (m, 1H), 8.18 (s, 1H), 8.05 (dd, J=8.9, 1.6 Hz, 1H), 7.69 (dd, J=8.7, 2.3 Hz, 2H), 7.60 (dd, J=8.8, 2.0 Hz, 1H), 7.50-7.41 (m, 4H), 4.76 (s, 2H), 4.63 (s, 1H), 4.61-4.49 (m, 3H), 4.37 (d, J=15.5 Hz, 1H), 4.14 (s, 3H), 3.93-3.89 (m, 1H), 3.83-3.75 (m, 2H), 3.66-3.57 (m, 2H), 3.28-3.16 (m, 3H), 2.52-2.44 (m, 4H), 2.39-2.20 (m, 4H), 2.20-2.15 (m, 2H), 2.12-2.06 (m, 1H), 1.86-1.78 (m, 1H), 1.74-1.67 (m, 2H), 1.64-1.56 (m, 4H), 1.38-1.32 (m, 4H), 1.04 (s, 9H). HRMS m/z [M+H]⁺ calcd for C₅₃H₆₈N₁₁O₆S⁺ 986.5069, found 986.5139.

### Example 47

### Synthesis of LQ076-90

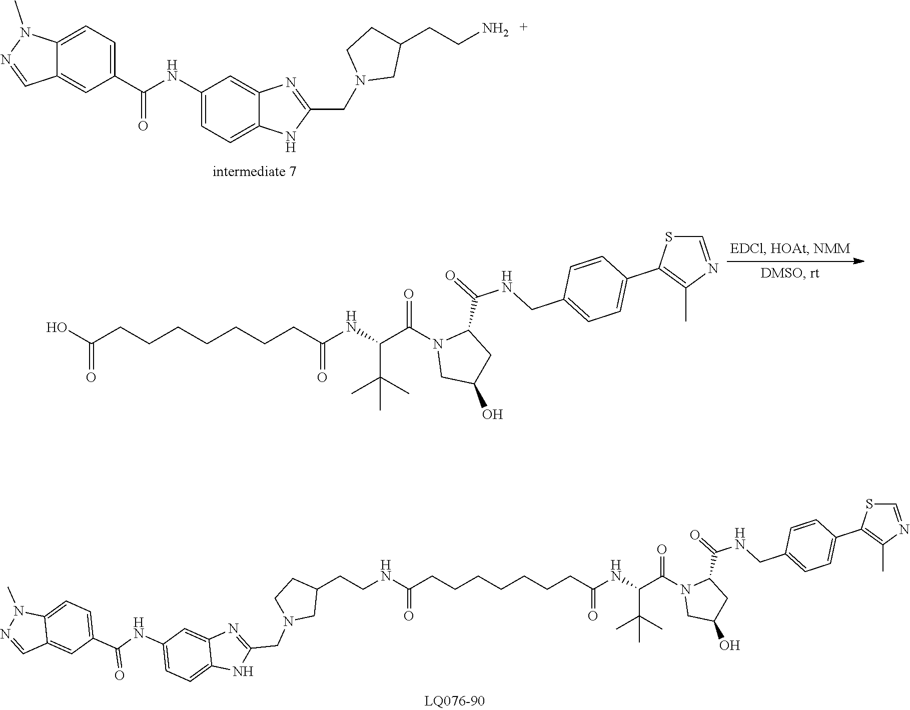

LQ076-90 was synthesized following the standard procedure for preparing LQ076-76 from intermediate 7 (13 mg, 0.02 mmol), 9-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-9-oxononanoic acid (12.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-90 was obtained as white solid in TFA salt form (18.7 mg, 76%). ¹H NMR (600 MHz, Methanol-d₄) δ 8.99 (s, 1H), 8.47 (s, 1H), 8.31 (s, 1H), 8.18 (s, 1H), 8.05 (dd, J=8.9, 1.6 Hz, 1H), 7.70-7.66 (m, 2H), 7.58 (dd, J=8.8, 1.9 Hz, 1H), 7.49-7.41 (m, 4H), 4.73 (s, 2H), 4.65-4.62 (m, 1H), 4.61-4.49 (m, 3H), 4.37 (d, J=15.5 Hz, 1H), 4.14 (s, 3H), 3.91 (d, J=10.9 Hz, 1H), 3.82-3.74 (m, 2H), 3.66-3.56 (m, 2H), 3.28-3.16 (m, 3H), 2.51-2.44 (m, 4H), 2.38-2.14 (m, 8H), 2.12-2.06 (m, 1H), 1.85-1.78 (m, 1H), 1.74-1.67 (m, 2H), 1.63-1.55 (m, 4H), 1.36-1.32 (m, 4H), 1.04 (s, 9H). HRMS m/z [M+H]⁺ calcd for C₅₄H₇₀N₁₁O₆S⁺ 1000.5226, found 1000.5303.

### Example 48

### Synthesis of LQ076-91

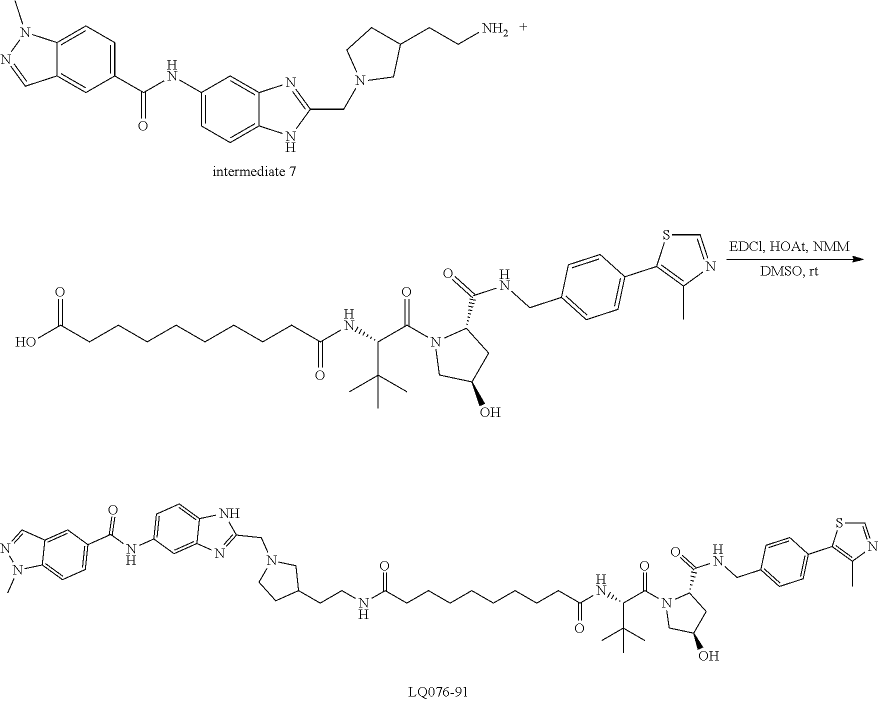

LQ076-91 was synthesized following the standard procedure for preparing LQ076-76 from intermediate 7 (13 mg, 0.02 mmol), 10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecanoic acid (12.7 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-91 was obtained as white solid in TFA salt form (18.8 mg, 76%). ¹H NMR (600 MHz, Methanol-d/4) δ 8.99 (s, 1H), 8.47 (s, 1H), 8.31 (d, J=1.9 Hz, 1H), 8.18 (d, J=0.9 Hz, 1H), 8.05 (dd, J=8.9, 1.7 Hz, 1H), 7.71-7.66 (m, 2H), 7.57 (dd, J=8.8, 1.9 Hz, 1H), 7.50-7.46 (m, 2H), 7.45-7.41 (m, 2H), 4.73 (s, 2H), 4.66-4.63 (m, 1H), 4.61-4.50 (m, 3H), 4.37 (d, J=15.5 Hz, 1H), 4.14 (s, 3H), 3.91 (d, J=11.0 Hz, 1H), 3.83-3.74 (m, 2H), 3.66-3.56 (m, 2H), 3.26-3.16 (m, 3H), 2.51-2.45 (m, 4H), 2.38-2.14 (m, 7H), 2.12-2.06 (m, 1H), 1.85-1.78 (m, 1H), 1.73-1.67 (m, 2H), 1.64-1.55 (N, 4H), 1.34-1.29 (m, 7H), 1.04 (s, 9H). HRMS m/z [M+H]⁺ calcd for C₅₅H₇₂N₁₁O₆S⁺ 1014.5382, found 1014.5464.

### Example 49

### Synthesis of LQ076-92

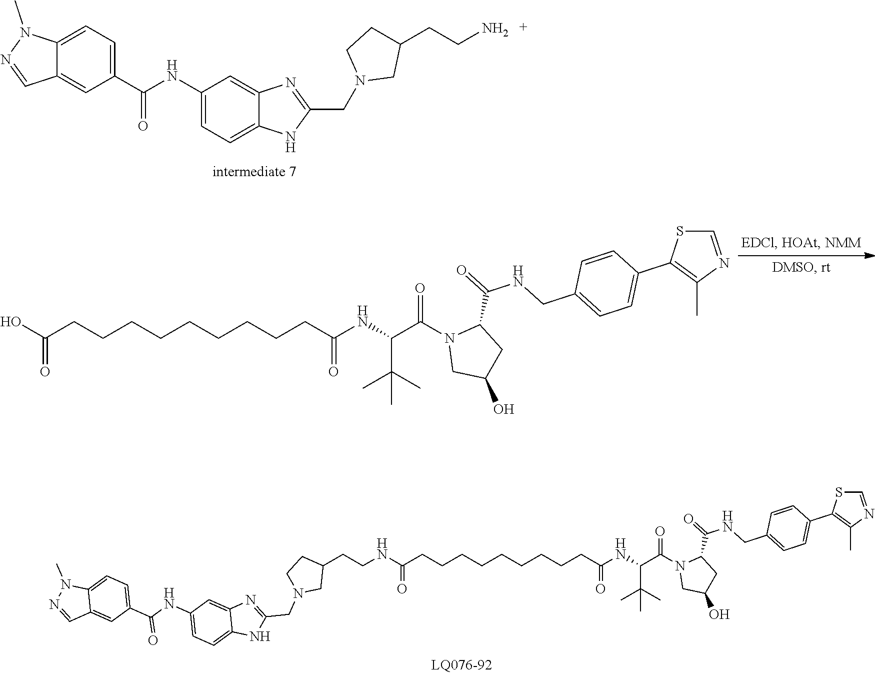

LQ076-92 was synthesized following the standard procedure for preparing LQ076-76 from intermediate 7 (13 mg, 0.02 mmol), 11-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-11-oxoundecanoic acid (13 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-92 was obtained as white solid in TFA salt form (20 mg, 79%). ¹H NMR (600 MHz, Methanol-d₄) δ 9.09 (s, 1H), 8.47 (s, 1H), 8.35 (d, J=2.0 Hz, 1H), 8.19 (s, 1H), 8.05 (dd, J=8.9, 1.7 Hz, 1H), 7.72-7.68 (m, 2H), 7.61 (dd, J=8.7, 1.9 Hz, 1H), 7.50-7.42 (m, 4H), 4.77 (s, 2H), 4.65-4.63 (m, 1H), 4.61-4.49 (m, 3H), 4.38 (d, J=15.5 Hz, 1H), 4.14 (s, 3H), 3.91 (d, J=11.0 Hz, 1H), 3.83-3.74 (m, 2H), 3.66-3.57 (m, 2H), 3.27-3.17 (m, 3H), 2.52-2.45 (m, 4H), 2.38-2.15 (m, 7H), 2.12-2.06 (m, 1H), 1.86-1.78 (m, 1H), 1.73-1.68 (m, 2H), 1.63-1.54 (m, 4H), 1.31-1.28 (m, 9H), 1.05 (s, 9H). HRMS m/z [M+H]⁺ calcd for C₅₆H₇₄N₁₁O₆S⁺ 1028.5539, found 1028.5597.

### Example 50

### Synthesis of LQ076-93

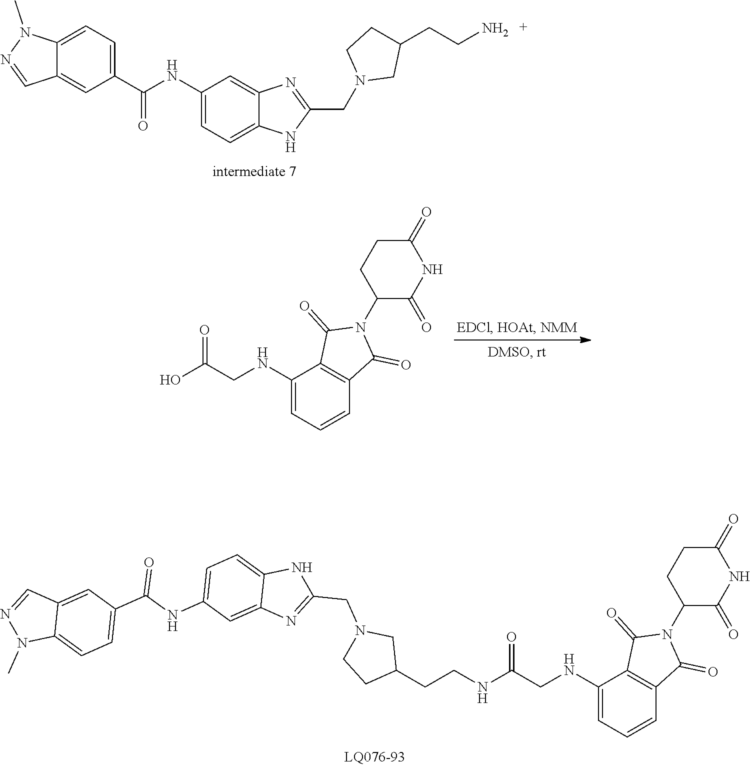

LQ076-93 was synthesized following the standard procedure for preparing LQ076-76 from intermediate 7 (13 mg, 0.02 mmol), (2-(2,6-dioxopiperidin-3-yl)-1,3-dioxoisoindolin-4-yl)glycine (6.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-93 was obtained as yellow solid in TFA salt form (12 mg, 63%). ¹H NMR (600 MHz, Methanol-d₄) δ 8.46 (s, 1H), 8.31-8.29 (m, 1H), 8.18 (s, 1H), 8.04 (dd, J=8.9, 1.6 Hz, 1H), 7.71-7.66 (m, 2H), 7.59-7.55 (m, 2H), 7.11 (d, J=7.1 Hz, 1H), 6.88 (d, J=8.5 Hz, 1H), 5.08 (dd, J=12.6, 5.4 Hz, 1H), 4.72 (s, 2H), 4.14 (s, 3H), 4.00 (s, 2H), 3.78-3.73 (m, 1H), 3.64-3.54 (m, 2H), 3.31-3.24 (m, 2H), 3.21-3.15 (m, 1H), 2.90-2.83 (m, 1H), 2.78-2.67 (m, 2H), 2.47-2.41 (m, 1H), 2.35-2.28 (m, 1H), 2.14-2.08 (m, 1H), 1.83-1.76 (m, 1H), 1.72-1.67 (m, 2H). HRMS m/z [M+H]⁺ calcd for C₃₈H₃₉N₁₀O₆⁺ 731.3049, found 731.3090.

### Example 51

### Synthesis of LQ076-94

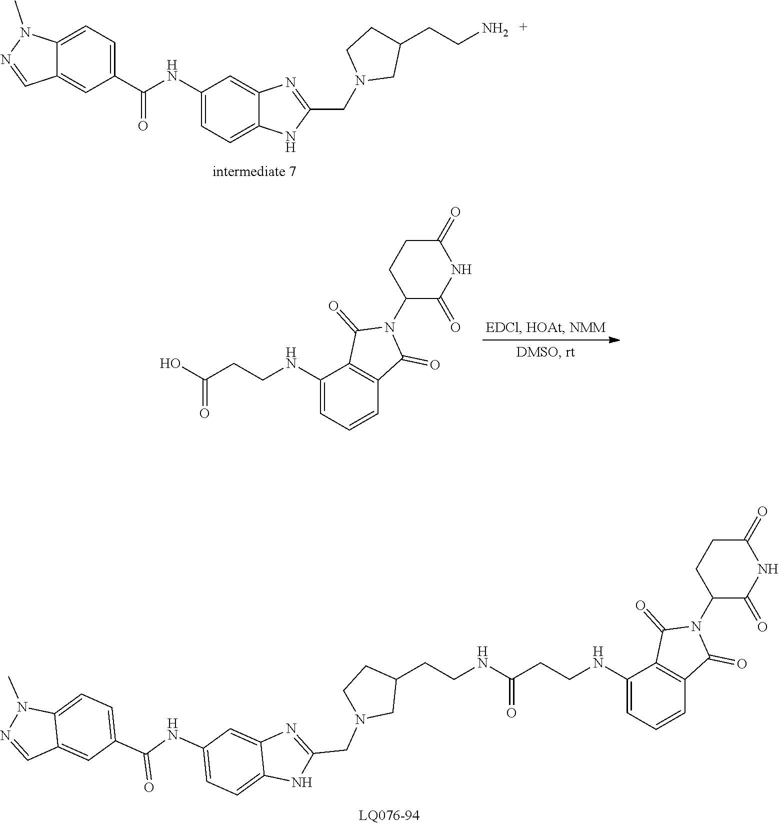

LQ076-94 was synthesized following the standard procedure for preparing LQ076-76 from intermediate 7 (13 mg, 0.02 mmol), 3-((2-(2,6-dioxopiperidin-3-yl)-1,3-dioxoisoindolin-4-yl)amino)propanoic acid (7.1 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-94 was obtained as yellow solid in TFA salt form (13.2 mg, 68%). ¹H NMR (600 MHz, Methanol-d₄) δ 8.46 (s, 1H), 8.30 (d, J=1.9 Hz, 1H), 8.18 (s, 1H), 8.04 (dd, J=8.9, 1.7 Hz, 1H), 7.71-7.67 (m, 2H), 7.58-7.54 (m, 2H), 7.10 (d, J=8.6 Hz, 1H), 7.05 (d, J=7.0 Hz, 1H), 5.04 (dd, J=12.5, 5.6 Hz, 1H), 4.73 (s, 2H), 4.14 (s, 3H), 3.75-3.50 (m, 5H), 3.29-3.14 (m, 3H), 2.87-2.78 (m, 1H), 2.75-2.66 (m, 2H), 2.56-2.50 (m, 2H), 2.46-2.39 (m, 1H), 2.32-2.25 (m, 1H), 2.12-2.07 (m, 1H), 1.78-1.71 (m, 1H), 1.65-1.59 (m, 2H). HRMS m/z [M+H]⁺ calcd for C₃₉H₄₁N₁₀O₆⁺ 745.3205, found 745.3248.

### Example 52

### Synthesis of LQ076-95

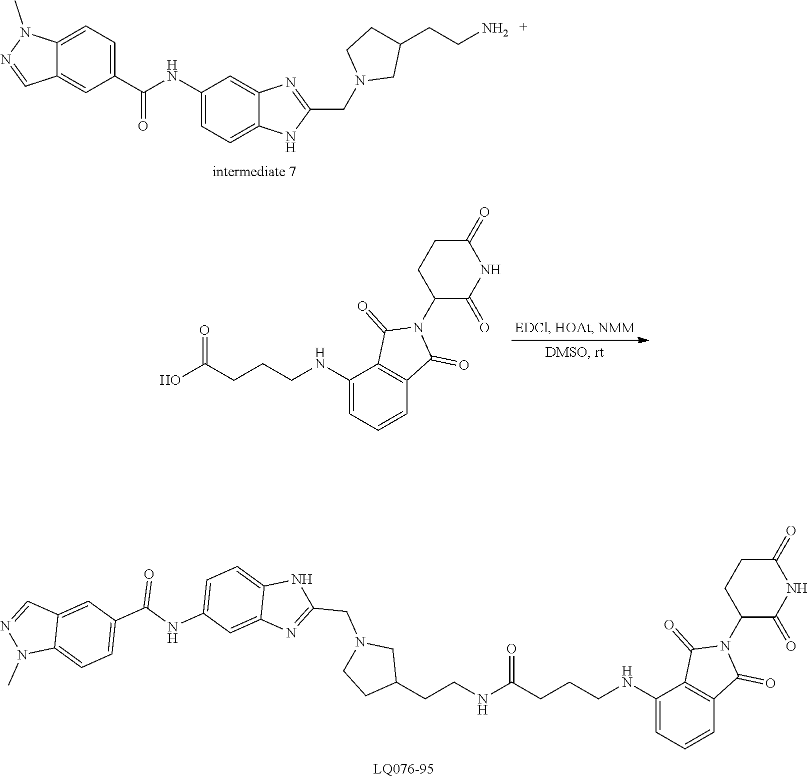

LQ076-95 was synthesized following the standard procedure for preparing LQ076-76 from intermediate 7 (13 mg, 0.02 mmol), 4-((2-(2,6-dioxopiperidin-3-yl)-1,3-dioxoisoindolin-4-yl)amino)butanoic acid (7.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-95 was obtained as yellow solid in TFA salt form (15.6 mg, 79%). ¹H NMR (600 MHz, Methanol-d₄) δ 8.46 (s, 1H), 8.33 (d, J=1.9 Hz, 1H), 8.18 (s, 1H), 8.04 (dd, J=8.9, 1.6 Hz, 1H), 7.71-7.67 (m, 2H), 7.58 (dd, J=8.7, 1.9 Hz, 1H), 7.53 (dd, J=8.6, 7.0 Hz, 1H), 7.06-7.01 (m, 2H), 5.05 (dd, J=12.7, 5.5 Hz, 1H), 4.74 (s, 2H), 4.14 (s, 3H), 3.77-3.72 (m, 1H), 3.63-3.55 (m, 2H), 3.37-3.34 (m, 2H), 3.24-3.16 (m, 3H), 2.89-2.82 (m, 1H), 2.77-2.67 (m, 2H), 2.49-2.43 (m, 1H), 2.36-2.28 (m, 3H), 2.13-2.08 (m, 1H), 1.97-1.91 (m, 2H), 1.83-1.77 (m, 1H), 1.69-1.65 (m, 2H). HRMS m/z [M+H]⁺ calcd for C₄₀H₄₃N₁₀O₆⁺ 759.3362, found 759.3401.

### Example 53

### Synthesis of LQ076-96

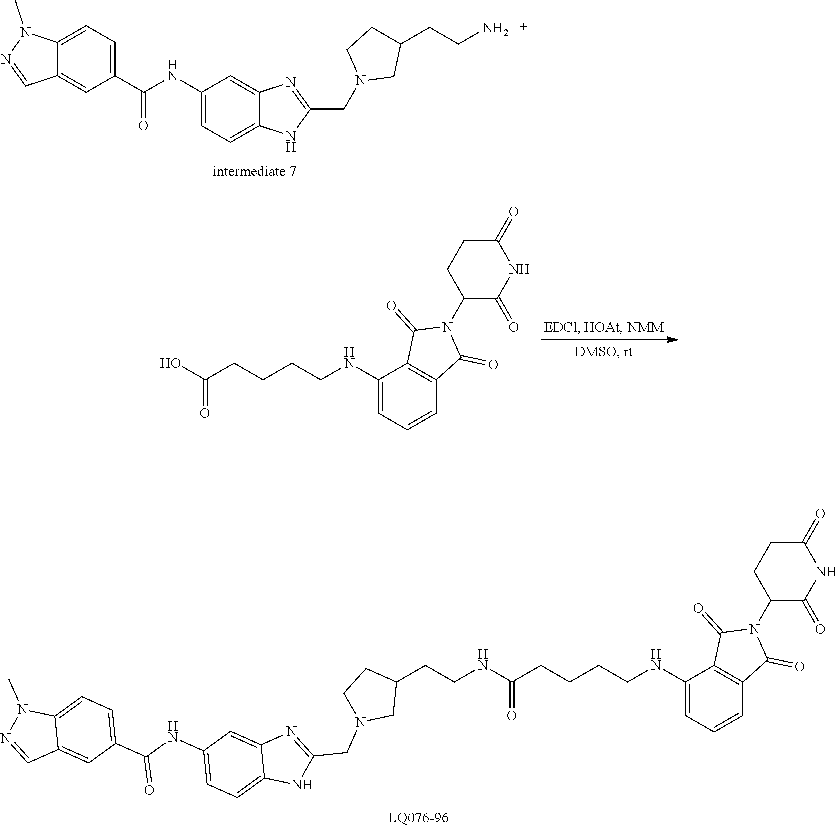

LQ076-96 was synthesized following the standard procedure for preparing LQ076-76 from intermediate 7 (13 mg, 0.02 mmol), 5-((2-(2,6-dioxopiperidin-3-yl)-1,3-dioxoisoindolin-4-yl)amino)pentanoic acid (7.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-96 was obtained as yellow solid in TFA salt form (15.4 mg, 77%). ¹H NMR (600 MHz, Methanol-d₄) δ 8.45 (s, 1H), 8.30 (d, J=2.0 Hz, 1H), 8.17 (s, 1H), 8.03 (dd, J=8.9, 1.7 Hz, 1H), 7.69-7.65 (m, 2H), 7.56 (dd, J=8.7, 2.0 Hz, 1H), 7.52 (dd, J=8.6, 7.1 Hz, 1H), 7.03-7.01 (m, 1H), 7.01-6.99 (m, 1H), 5.03 (dd, J=12.8, 5.4 Hz, 1H), 4.71 (s, 2H), 4.13 (s, 3H), 3.76-3.72 (m, 1H), 3.63-3.54 (m, 2H), 3.31-3.29 (m, 2H), 3.25-3.15 (m, 3H), 2.87-2.80 (m, 1H), 2.76-2.65 (m, 2H), 2.49-2.43 (m, 1H), 2.35-2.29 (m, 1H), 2.23 (t, J=7.2 Hz, 2H), 2.11-2.06 (m, 1H), 1.83-1.77 (m, 1H), 1.74-1.62 (m, 6H). HRMS m/z [M+H]⁺ calcd for C₄₁H₄₅N₁₀O₆⁺ 773.3518, found 773.3530.

### Example 54

### Synthesis of LQ076-97

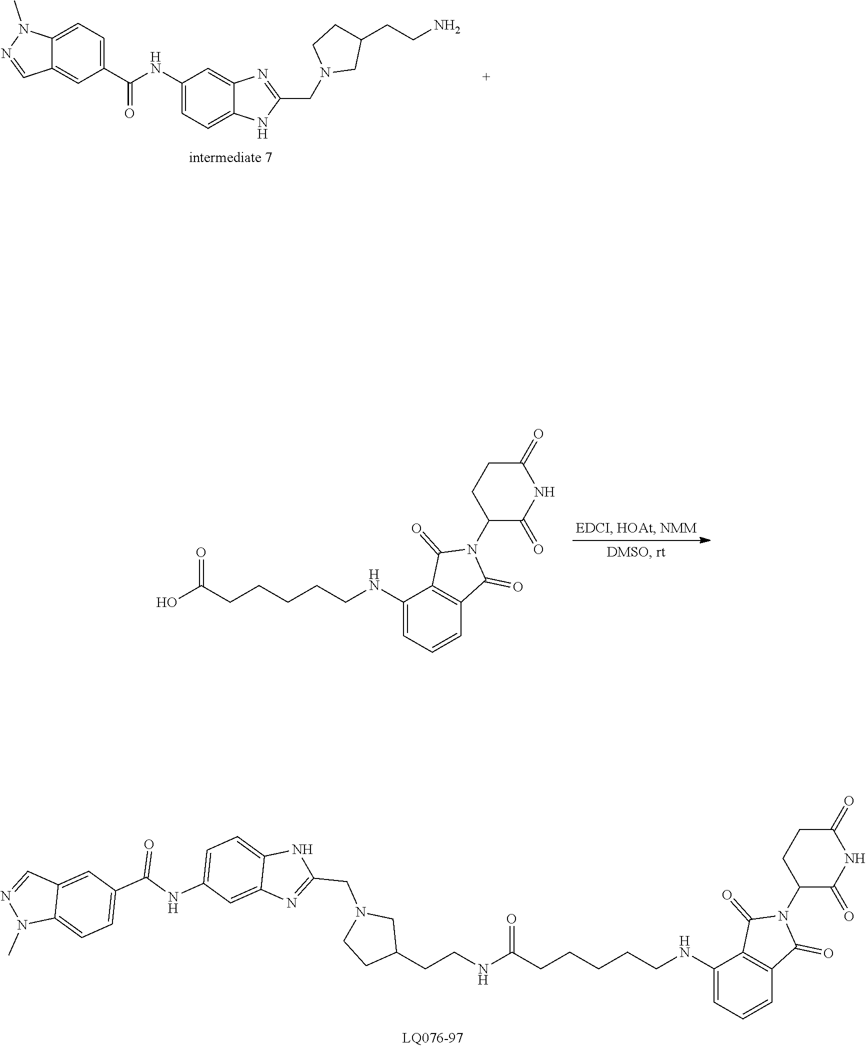 [SMILES: Cn1ncc2cc(C(=O)Nc3ccc4[nH]c(CN5CCC(CCN)C5)nc4c3)ccc21 ; O=C(O)CCCCCNc1cccc2c1C(=O)N(C1CCC(=O)NC1=O)C2=O ; Cn1ncc2cc(C(=O)Nc3ccc4[nH]c(CN5CCC(CCNC(=O)CCCCCNc6cccc7c6C(=O)N(C6CCC(=O)NC6=O)C7=O)C5)nc4c3)ccc21]

LQ076-97 was synthesized following the standard procedure for preparing LQ076-76 from intermediate 7 (13 mg, 0.02 mmol), 6-((2-(2,6-dioxopiperidin-3-yl)-1,3-dioxoisoindolin-4-yl)amino)hexanoic acid (7.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-97 was obtained as yellow solid in TFA salt form (14.9 mg, 73%). ¹H NMR (600 MHz, Methanol-d₄) δ 8.45 (s, 1H), 8.29 (d, J=1.9 Hz, 1H), 8.17 (d, J=0.9 Hz, 1H), 8.03 (dd, J=8.8, 1.7 Hz, 1H), 7.69-7.64 (m, 2H), 7.55 (dd, J=8.7, 2.0 Hz, 1H), 7.50 (dd, J=8.5, 7.1 Hz, 1H), 7.02-6.97 (m, 2H), 5.05 (dd, J=12.5, 5.7 Hz, 1H), 4.70 (s, 2H), 4.13 (s, 3H), 3.77-3.71 (m, 1H), 3.64-3.53 (m, 2H), 3.29 (t, J=6.9 Hz, 2H), 3.25-3.20 (m, 2H), 3.20-3.13 (m, 1H), 2.90-2.81 (m, 1H), 2.78-2.66 (m, 2H), 2.50-2.41 (m, 1H), 2.37-2.28 (m, 1H), 2.19 (t, J=7.4 Hz, 2H), 2.14-2.07 (m, 1H), 1.84-1.74 (m, 1H), 1.71-1.59 (m, 6H), 1.46-1.38 (m, 2H). HRMS m/z [M+H]⁺ calcd for C₄₂H₄₇N₁₀O₆⁺ 787.3675, found 787.3710.

### Example 55

### Synthesis of LQ076-98

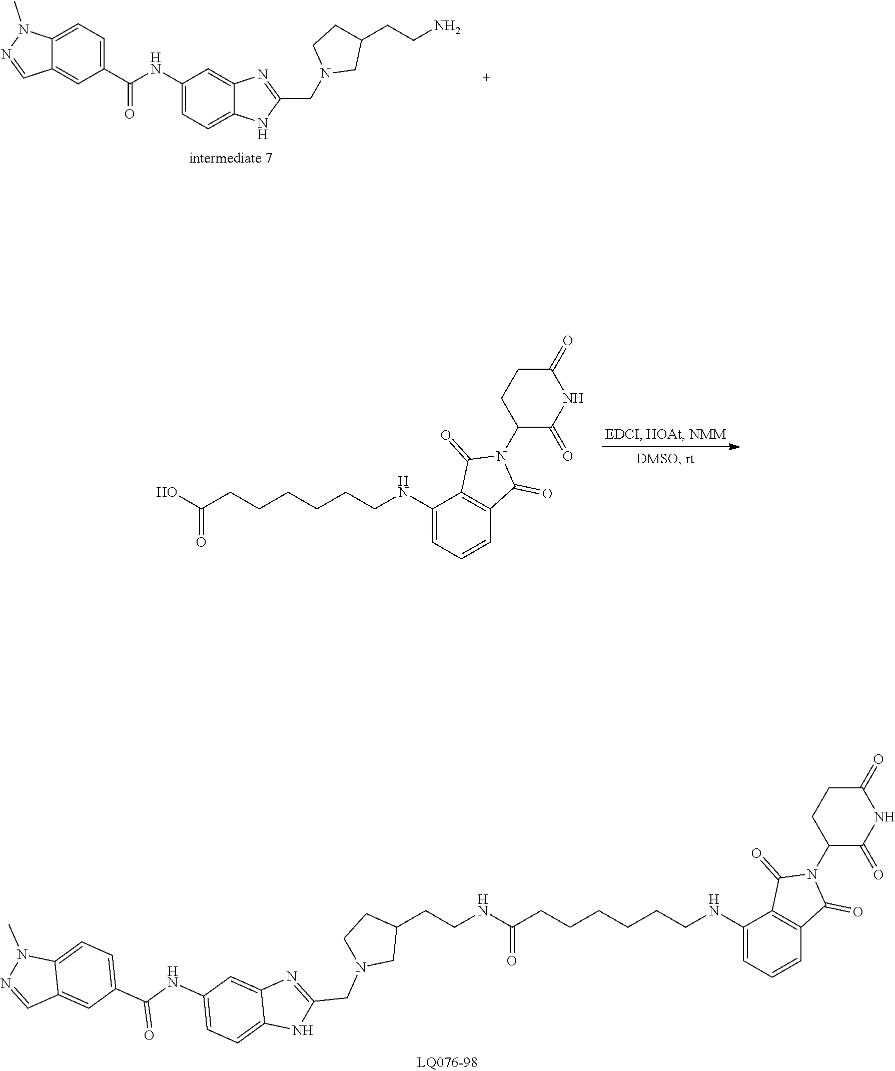 [SMILES: Cn1ncc2cc(C(=O)Nc3ccc4[nH]c(CN5CCC(CCN)C5)nc4c3)ccc21 ; O=C(O)CCCCCCNc1cccc2c1C(=O)N(C1CCC(=O)NC1=O)C2=O ; Cn1ncc2cc(C(=O)Nc3ccc4[nH]c(CN5CCC(CCNC(=O)CCCCCCNc6cccc7c6C(=O)N(C6CCC(=O)NC6=O)C7=O)C5)nc4c3)ccc21]

LQ076-98 was synthesized following the standard procedure for preparing LQ076-76 from intermediate 7 (13 mg, 0.02 mmol), 7-((2-(2,6-dioxopiperidin-3-yl)-1,3-dioxoisoindolin-4-yl)amino)heptanoic acid (8.6 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-98 was obtained as yellow solid in TFA salt form (16.1 mg, 78%). ¹H NMR (800 MHz, Methanol-d₄) δ 8.46 (s, 1H), 8.30 (s, 1H), 8.17 (s, 1H), 8.04 (d, J=8.8 Hz, 1H), 7.67 (dd, J=8.7, 3.8 Hz, 2H), 7.57 (d, J=8.6 Hz, 1H), 7.53-7.50 (m, 1H), 7.02-6.98 (m, 2H), 5.06 (dd, J=12.7, 5.6 Hz, 1H), 4.71 (s, 2H), 4.13 (s, 3H), 3.75 (t, J=9.9 Hz, 1H), 3.64-3.55 (m, 2H), 3.30-3.15 (m, 5H), 2.89-2.83 (m, 1H), 2.77-2.69 (m, 2H), 2.49-2.45 (m, 1H), 2.36-2.32 (m, 1H), 2.19-2.16 (m, 2H), 2.13-2.10 (m, 1H), 1.83-1.79 (m, 1H), 1.72-1.58 (m, 6H), 1.45-1.40 (m, 2H), 1.39-1.35 (m, 2H). HRMS m/z [M+H]⁺ calcd for C₄₃H₄₉N₁₀O₆⁺ 801.3831, found 801.3799.

### Example 56

### Synthesis of LQ076-99

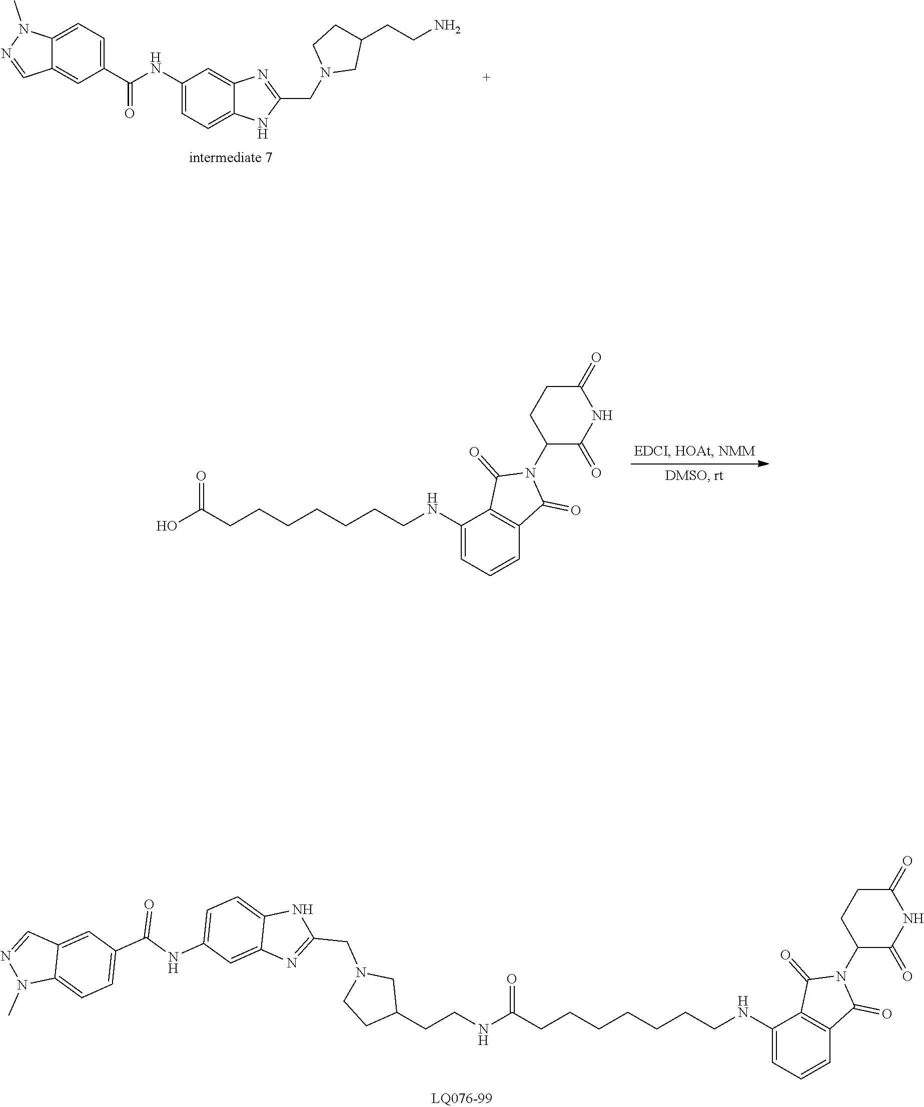 [SMILES: Cn1ncc2cc(C(=O)Nc3ccc4[nH]c(CN5CCC(CCN)C5)nc4c3)ccc21 ; O=C(O)CCCCCCCNc1cccc2c1C(=O)N(C1CCC(=O)NC1=O)C2=O ; Cn1ncc2cc(C(=O)Nc3ccc4[nH]c(CN5CCC(CCNC(=O)CCCCCCCNc6cccc7c6C(=O)N(C6CCC(=O)NC6=O)C7=O)C5)nc4c3)ccc21]

LQ076-99 was synthesized following the standard procedure for preparing LQ076-76 from intermediate 7 (13 mg, 0.02 mmol), 8-((2-(2,6-dioxopiperidin-3-yl)-1,3-dioxoisoindolin-4-yl)amino)octanoic acid (8.7 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-99 was obtained as yellow solid in TFA salt form (14.3 mg, 69%). ¹H NMR (600 MHz, Methanol-d₄) δ 8.45 (s, 1H), 8.33 (d, J=1.6 Hz, 1H), 8.16 (s, 1H), 8.03 (dd, J=8.8, 1.7 Hz, 1H), 7.70-7.64 (m, 2H), 7.60 (dd, J=8.7, 2.0 Hz, 1H), 7.50 (dd, J=8.6, 7.1 Hz, 1H), 7.01-6.96 (m, 2H), 5.05 (dd, J=12.8, 5.5 Hz, 1H), 4.76 (s, 2H), 4.12 (s, 3H), 3.78-3.72 (m, 1H), 3.65-3.55 (m, 2H), 3.29-3.16 (m, 5H), 2.89-2.81 (m, 1H), 2.77-2.66 (m, 2H), 2.51-2.43 (m, 1H), 2.37-2.30 (m, 1H), 2.16 (t, J=7.5 Hz, 2H), 2.13-2.07 (m, 1H), 1.85-1.76 (m, 1H), 1.71-1.67 (m, 2H), 1.66-1.55 (m, 4H), 1.44-1.28 (m, 6H). HRMS m/z [M+H]⁺ calcd for C₄₄H₅₁N₁₀O₆⁺ 815.3988, found 815.4019.

### Example 57

### Synthesis of LQ076-100

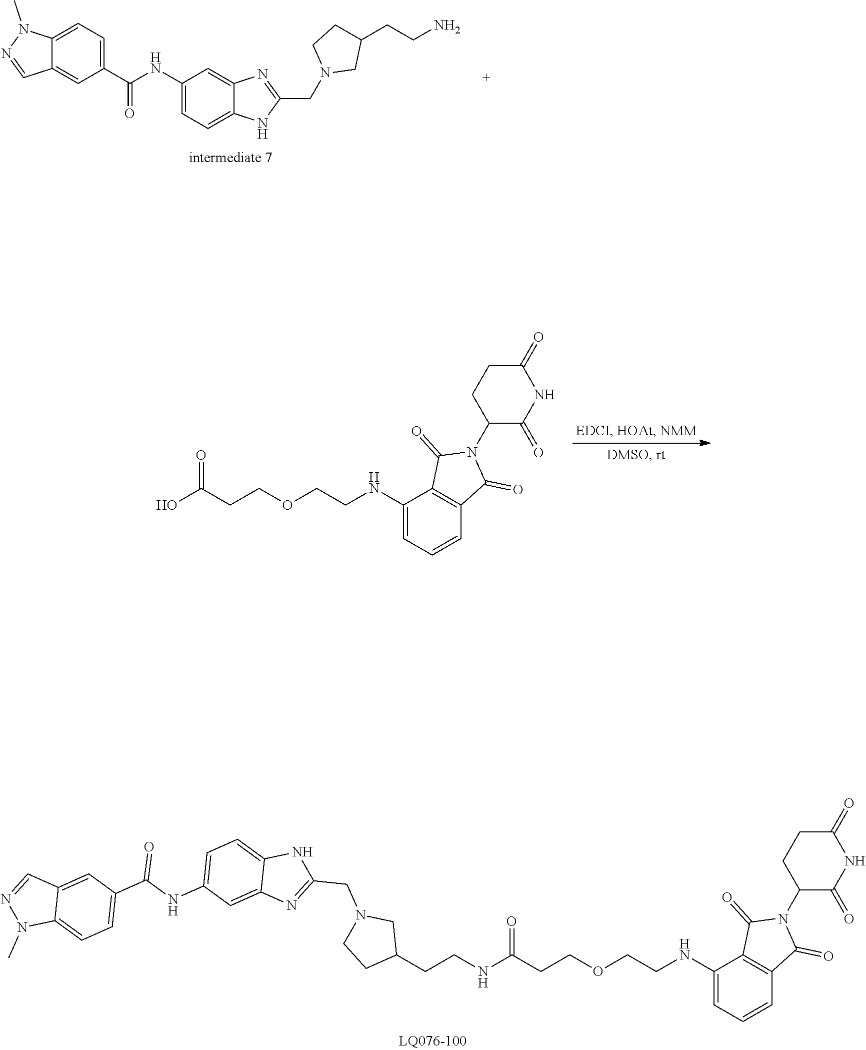 [SMILES: Cn1ncc2cc(C(=O)Nc3ccc4[nH]c(CN5CCC(CCN)C5)nc4c3)ccc21 ; O=C(O)CCOCCNc1cccc2c1C(=O)N(C1CCC(=O)NC1=O)C2=O ; Cn1ncc2cc(C(=O)Nc3ccc4[nH]c(CN5CCC(CCNC(=O)CCOCCNc6cccc7c6C(=O)N(C6CCC(=O)NC6=O)C7=O)C5)nc4c3)ccc21]

LQ076-100 was synthesized following the standard procedure for preparing LQ076-76 from intermediate 7 (13 mg, 0.02 mmol), 3-(2-((2-(2,6-dioxopiperidin-3-yl)-1,3-dioxoisoindolin-4-yl)amino)ethoxy)propanoic acid (8.1 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-100 was obtained as yellow solid in TFA salt form (15.1 mg, 74%). ¹H NMR (600 MHz, Methanol-d₄) δ 8.46 (s, 1H), 8.30 (d, J=1.9 Hz, 1H), 8.18 (s, 1H), 8.04 (dd, J=8.8, 1.7 Hz, 1H), 7.70-7.66 (m, 2H), 7.56 (dd, J=8.7, 1.9 Hz, 1H), 7.54-7.50 (m, 1H), 7.07 (d, J=8.6 Hz, 1H), 7.02 (d, J=7.1 Hz, 1H), 5.05 (dd, J=12.8, 5.5 Hz, 1H), 4.70 (s, 2H), 4.14 (s, 3H), 3.77-3.66 (m, 5H), 3.61-3.52 (m, 2H), 3.47 (t, J=5.1 Hz, 2H), 3.24-3.11 (m, 3H), 2.89-2.82 (m, 1H), 2.78-2.66 (m, 2H), 2.45 (t, J=5.8 Hz, 3H), 2.32-2.25 (m, 1H), 2.14-2.09 (m, 1H), 1.79-1.72 (m, 1H), 1.65-1.60 (m, 2H). HRMS m/z [M+H]⁺ calcd for C₄₁H₄₅N₁₀O₇⁺ 789.3467, found 789.3511.

### Example 58

### Synthesis of LQ076-101

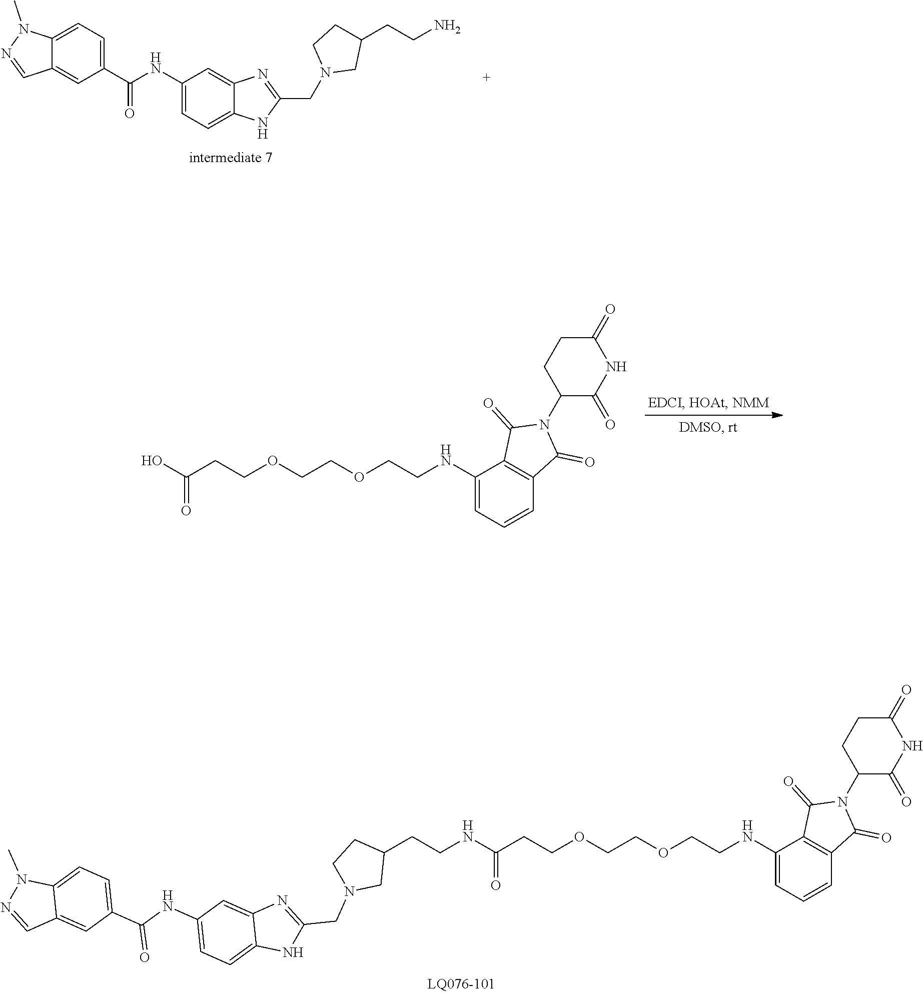 [SMILES: Cn1ncc2cc(C(=O)Nc3ccc4[nH]c(CN5CCC(CCN)C5)nc4c3)ccc21 ; O=C(O)CCOCCOCCNc1cccc2c1C(=O)N(C1CCC(=O)NC1=O)C2=O ; Cn1ncc2cc(C(=O)Nc3ccc4[nH]c(CN5CCC(CCNC(=O)CCOCCOCCNc6cccc7c6C(=O)N(C6CCC(=O)NC6=O)C7=O)C5)nc4c3)ccc21]

LQ076-101 was synthesized following the standard procedure for preparing LQ076-76 from intermediate 7 (13 mg, 0.02 mmol), 3-(2-(2-((2-(2,6-dioxopiperidin-3-yl)-1,3-dioxoisoindolin-4-yl)amino)ethoxy)ethoxy)propanoic acid (9.1 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-101 was obtained as yellow solid in TFA salt form (16.1 mg, 76%). ¹H NMR (600 MHz, Methanol-d₄) δ 8.46 (s, 1H), 8.30 (d, J=1.9 Hz, 1H), 8.17 (s, 1H), 8.04 (dd, J=8.8, 1.6 Hz, 1H), 7.70-7.65 (m, 2H), 7.57 (dd, J=8.7, 2.0 Hz, 1H), 7.53-7.49 (m, 1H), 7.05-7.01 (m, 2H), 5.05 (dd, J=12.8, 5.5 Hz, 1H), 4.72 (s, 2H), 4.13 (s, 3H), 3.75-3.69 (m, 5H), 3.66-3.52 (m, 6H), 3.46 (t, J=5.2 Hz, 2H), 3.26-3.12 (m, 3H), 2.89-2.80 (m, 1H), 2.77-2.66 (m, 2H), 2.49-2.40 (m, 3H), 2.33-2.26 (m, 1H), 2.14-2.09 (m, 1H), 1.80-1.73 (m, 1H), 1.69-1.64 (m, 2H). HRMS m/z [M+H]⁺ calcd for C₄₃H₄₉N₁₀O₈⁺ 833.3729, found 833.3785.

### Example 59

### Synthesis of LQ076-102

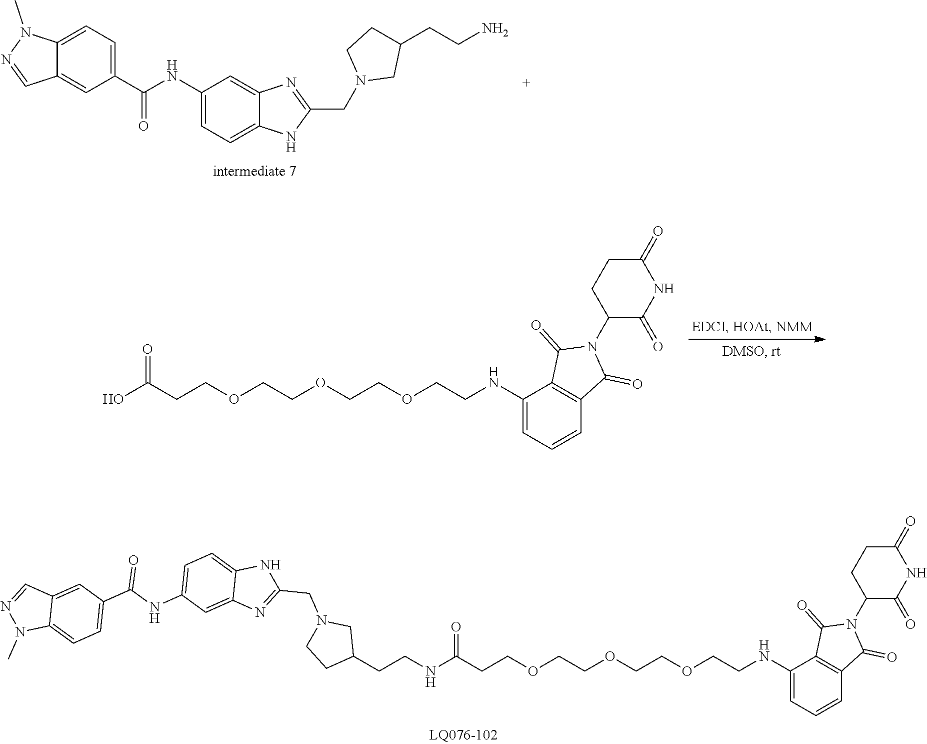 [SMILES: Cn1ncc2cc(C(=O)Nc3ccc4[nH]c(CN5CCC(CCN)C5)nc4c3)ccc21 ; O=C(O)CCOCCOCCOCCNc1cccc2c1C(=O)N(C1CCC(=O)NC1=O)C2=O ; Cn1ncc2cc(C(=O)Nc3ccc4[nH]c(CN5CCC(CCNC(=O)CCOCCOCCOCCNc6cccc7c6C(=O)N(C6CCC(=O)NC6=O)C7=O)C5)nc4c3)ccc21]

LQ076-102 was synthesized following the standard procedure for preparing LQ076-76 from intermediate 7 (13 mg, 0.02 mmol), 3-(2-(2-(2-((2-(2,6-dioxopiperidin-3-yl)-1,3-dioxoisoindolin-4-yl)amino)ethoxy)ethoxy)ethoxy)propanoic acid (9.1 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-102 was obtained as yellow solid in TFA salt form (15.7 mg, 71%). ¹H NMR (600 MHz, Methanol-d₄) δ 8.46 (s, 1H), 8.30 (d, J=1.9 Hz, 1H), 8.17 (s, 1H), 8.04 (dd, J=8.8, 1.6 Hz, 1H), 7.70-7.65 (m, 2H), 7.57 (dd, J=8.7, 2.0 Hz, 1H), 7.51 (dd, J=8.5, 7.1 Hz, 1H), 7.04-7.00 (m, 2H), 5.05 (dd, J=12.9, 5.5 Hz, 1H), 4.73 (s, 2H), 4.13 (s, 3H), 3.78-3.54 (m, 15H), 3.45 (t, J=5.1 Hz, 2H), 3.29-3.15 (m, 3H), 2.89-2.82 (m, 1H), 2.77-2.66 (m, 2H), 2.51-2.45 (m, 1H), 2.41 (t, J=6.0 Hz, 2H), 2.36-2.29 (m, 1H), 2.14-2.08 (m, 1H), 1.82-1.76 (m, 1H), 1.71-1.65 (m, 2H). HRMS m/z [M+H]⁺ calcd for C₄₅H₅₃N₁₀O₉⁺ 877.3991, found 877.4037.

### Example 60

### Synthesis of LQ076-103

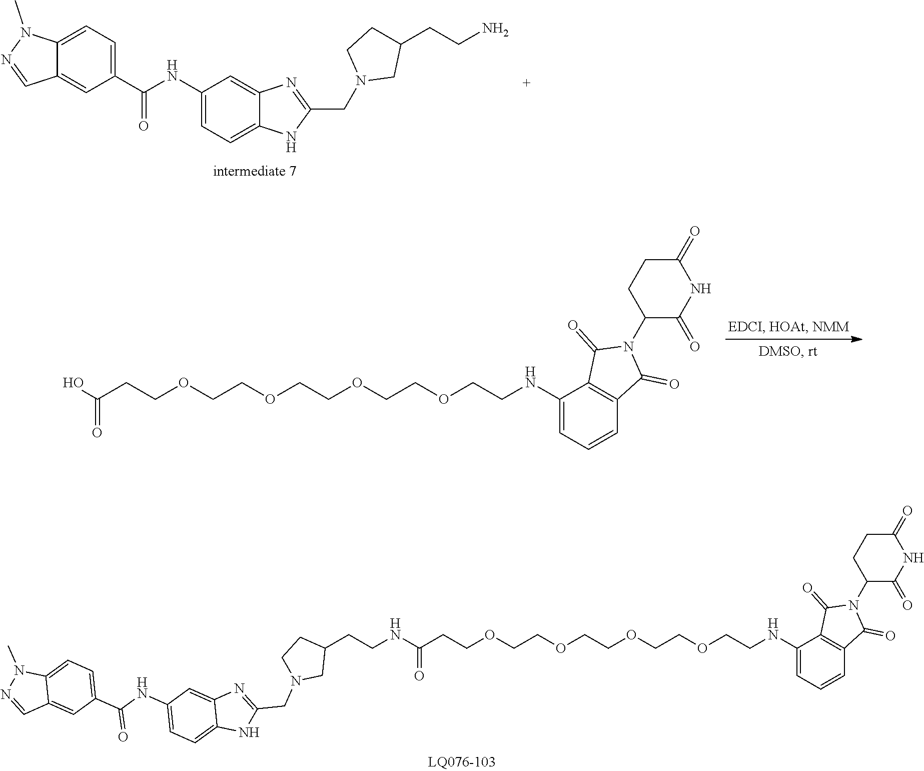 [SMILES: Cn1ncc2cc(C(=O)Nc3ccc4[nH]c(CN5CCC(CCN)C5)nc4c3)ccc21 ; O=C(O)CCOCCOCCOCCOCCNc1cccc2c1C(=O)N(C1CCC(=O)NC1=O)C2=O ; Cn1ncc2cc(C(=O)Nc3ccc4[nH]c(CN5CCC(CCNC(=O)CCOCCOCCOCCOCCNc6cccc7c6C(=O)N(C6CCC(=O)NC6=O)C7=O)C5)nc4c3)ccc21]

LQ076-103 was synthesized following the standard procedure for preparing LQ076-76 from intermediate 7 (13 mg, 0.02 mmol), 1-((2-(2,6-dioxopiperidin-3-yl)-1,3-dioxoisoindolin-4-yl)amino)-3,6,9,12-tetraoxapentadecan-15-oic acid (10.7 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-103 was obtained as yellow solid in TFA salt form (16.2 mg, 70%). ¹H NMR (600 MHz, Methanol-d₄) δ 8.46 (s, 1H), 8.32 (s, 1H), 8.18 (s, 1H), 8.04 (dd, J=8.8, 1.6 Hz, 1H), 7.70-7.66 (m, 2H), 7.58 (dd, J=8.7, 2.0 Hz, 1H), 7.54-7.48 (m, 1H), 7.05-7.00 (m, 2H), 5.05 (dd, J=12.8, 5.4 Hz, 1H), 4.74 (s, 2H), 4.13 (s, 3H), 3.75 (t, J=9.8 Hz, 1H), 3.72-3.68 (m, 4H), 3.66-3.55 (m, 14H), 3.45 (t, J=5.2 Hz, 2H), 3.28-3.15 (m, 3H), 2.89-2.82 (m, 1H), 2.77-2.67 (m, 2H), 2.49 (s, 1H), 2.41 (t, J=6.0 Hz, 2H), 2.36-2.30 (m, 1H), 2.14-2.08 (m, 1H), 1.83-1.76 (m, 1H), 1.71-1.66 (m, 2H). HRMS m/z [M+H]⁺ calcd for C₄₇H₅₇N₁₀O₁₀⁺ 921.4524, found 921.4546.

### Example 61

### Synthesis of LQ076-104

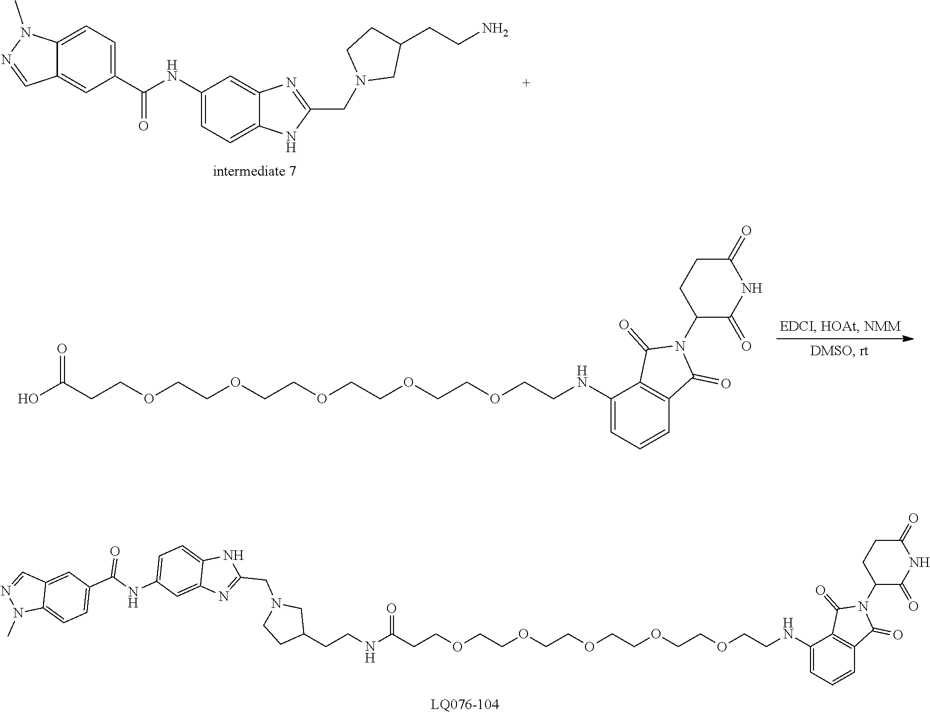 [SMILES: Cn1ncc2cc(C(=O)Nc3ccc4[nH]c(CN5CCC(CCN)C5)nc4c3)ccc21 ; O=C(O)CCOCCOCCOCCOCCOCCNc1cccc2c1C(=O)N(C1CCC(=O)NC1=O)C2=O ; Cn1ncc2cc(C(=O)Nc3ccc4[nH]c(CN5CCC(CCNC(=O)CCOCCOCCOCCOCCOCCNc6cccc7c6C(=O)N(C6CCC(=O)NC6=O)C7=O)C5)nc4c3)ccc21]

LQ076-104 was synthesized following the standard procedure for preparing LQ076-76 from intermediate 7 (13 mg, 0.02 mmol), 1-((2-(2,6-dioxopiperidin-3-yl)-1,3-dioxoisoindolin-4-yl)amino)-3,6,9,12,15-pentaoxaoctadecan-18-oic acid (11.7 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-104 was obtained as yellow solid in TFA salt form (16.7 mg, 70%). ¹H NMR (600 MHz, Methanol-d₄) δ 8.46 (s, 1H), 8.36 (d, J=1.9 Hz, 1H), 8.17 (s, 1H), 8.04 (dd, J=8.8, 1.6 Hz, 1H), 7.72-7.64 (m, 3H), 7.62 (dd, J=8.7, 2.1 Hz, 1H), 7.53-7.47 (m, 1H), 7.04-7.00 (m, 2H), 5.05 (dd, J=12.9, 5.5 Hz, 1H), 4.80 (s, 2H), 4.13 (s, 3H), 3.83-3.75 (m, 1H), 3.73-3.67 (m, 4H), 3.67-3.51 (m, 18H), 3.45 (t, J=5.2 Hz, 2H), 3.29-3.17 (m, 3H), 2.90-2.82 (m, 1H), 2.78-2.66 (m, 2H), 2.55-2.47 (m, 1H), 2.42 (t, J=6.2 Hz, 2H), 2.38-2.31 (m, 1H), 2.15-2.09 (m, 1H), 1.86-1.77 (m, 1H), 1.74-1.67 (m, 2H). HRMS m/z [M+H]⁺ calcd for C₄₉H₆₁N₁₀O₁₁⁺ 965.4516, found 965.4554.

### Example 62

### Synthesis of Intermediate 10

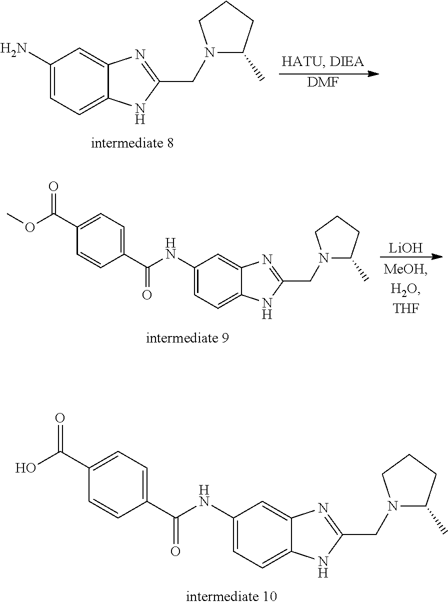

### Intermediate 9: Methyl (S)-4-((2-((2-methylpyrrolidin-1-yl)methyl)-1H-benzo[d]imidazol-5-yl)carbamoyl)benzoate

A solution of intermediate 8 (Moustakim et al., 2018b) (579 mg, 3.2 mmol) was dissolved in DMF and treated with 4-(Methoxycarbonyl)benzoic acid (740 mg, 3.2 mmol), HATU (1.4 g, 3.8 mmol) and DIEA (845 μL, 4.8 mmol). After being stirring 1 h at room temperature, the reaction mixture was poured into ice water, aqueous phase was extracted with ethyl acetate. The combined organic phase was washed with brine twice, dried and concentrated. The resulting residue was purified by silica gel flash chromatography to give the compound as grey solid (880 mg, 54%).

¹H NMR (600 MHz, Methanol-d₄) δ 8.31 (d, J=2.0 Hz, 1H), 8.19-8.14 (m, 2H), 8.08-8.03 (m, 2H), 7.69 (d, J=8.8 Hz, 1H), 7.59 (dd, J=8.7, 2.0 Hz, 1H), 4.84 (d, J=14.6 Hz, 1H), 4.61 (d, J=14.6 Hz, 1H), 3.97 (s, 3H), 3.79-3.70 (m, 2H), 3.50-3.43 (m, 1H), 2.44-2.35 (m, 1H), 2.19-2.06 (m, 2H), 1.88-1.78 (m, 1H), 1.51 (d, J=6.5 Hz, 3H). MS (ESI): m/z 393.3 [M+H]⁺.

### Intermediate 10: (S)-4-((2-((2-methylpyrrolidin-1-yl)methyl)-1H-benzo[d]imidazol-5-yl)carbamoyl)benzoic acid

Intermediate 10 was synthesized according to the procedures for the preparation of intermediate 4 as a white solid in 88% yield. ¹H NMR (600 MHz, Methanol-d₄) δ 8.30 (d, J=1.9 Hz, 1H), 8.20-8.15 (m, 2H), 8.08-8.03 (m, 2H), 7.68 (d, J=8.7 Hz, 1H), 7.57 (dd, J=8.8, 2.0 Hz, 1H), 4.81 (d, J=14.6 Hz, 1H), 4.57 (d, J=14.6 Hz, 1H), 3.79-3.70 (m, 2H), 3.50-3.43 (m, 1H), 2.44-2.35 (m, 1H), 2.20-2.05 (m, 2H), 1.87-1.79 (m, 1H), 1.51 (d, J=6.5 Hz, 3H). MS (ESI): m/z 379.3 [M+H]⁺.

### Example 63

### Synthesis of LQ076-105

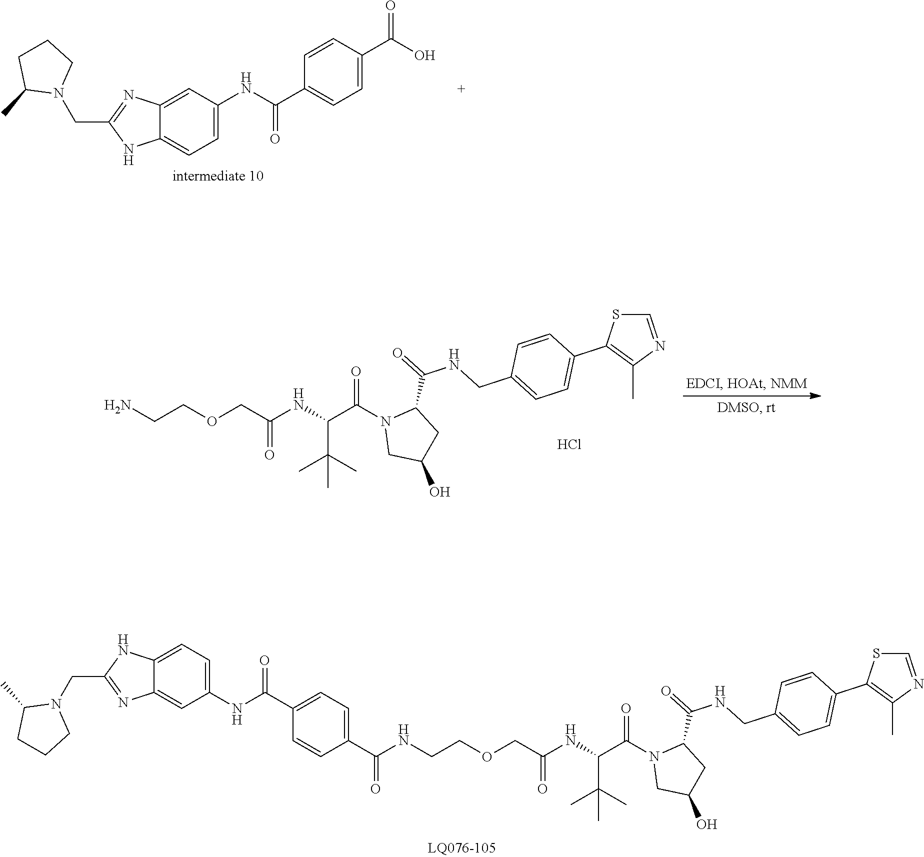 [SMILES: C[C@H]1CCCN1Cc1nc2cc(NC(=O)c3ccc(C(=O)O)cc3)ccc2[nH]1 ; Cc1ncsc1-c1ccc(CNC(=O)[C@@H]2C[C@@H](O)CN2C(=O)[C@@H](NC(=O)COCCN)C(C)(C)C)cc1 ; Cl ; Cc1ncsc1-c1ccc(CNC(=O)[C@@H]2C[C@@H](O)CN2C(=O)[C@@H](NC(=O)COCCNC(=O)c2ccc(C(=O)Nc3ccc4[nH]c(CN5CCC[C@@H]5C)nc4c3)cc2)C(C)(C)C)cc1]

To a solution of Intermediate 10 (10 mg, 0.02 mmol) in DMSO (1 mL) were added (2S,4R)-1-((S)-2-(2-(2-aminoethoxy)acetamido)-3,3-dimethylbutanoyl)-4-hydroxy-N-(4-(4-methylthiazol-5-yl)benzyl)pyrrolidine-2-carboxamide (11.5 mg, 0.02 mmol, 1.0 equiv), EDCI (1-ethyl-3-(3-dimethylaminopropyl)carbodiimide) (5.8 mg, 0.03 mmol, 1.5 equiv), HOAt (1-hydroxy-7-azabenzo-triazole) (4.1 mg, 0.03 mmol, 1.5 equiv), and NMM (N-Methylmorpholine) (6.1 mg, 0.06 mmol, 3.0 equiv). After being stirred overnight at room temperature, the resulting mixture was purified by preparative HPLC (5%-60% acetonitrile/0.1% TFA in H₂O) to afford LQ076-105 as white solid in TFA salt form (19.2 mg, 86%). ¹H NMR (600 MHz, Methanol-d₄) δ 9.01 (s, 1H), 8.30 (d, J=1.6 Hz, 1H), 8.04-8.01 (m, 4H), 7.68 (d, J=8.8 Hz, 1H), 7.57 (dd, J=8.7, 2.0 Hz, 1H), 7.47-7.38 (m, 4H), 4.83 (d, J=14.6 Hz, 1H), 4.75-4.72 (m, 1H), 4.62-4.50 (m, 4H), 4.38 (d, J=15.5 Hz, 1H), 4.15-4.05 (m, 2H), 3.85-3.63 (m, 8H), 3.51-3.42 (m, 1H), 2.45 (s, 3H), 2.42-2.36 (m, 1H), 2.29-2.23 (m, 1H), 2.18-2.07 (m, 3H), 1.86-1.79 (m, 1H), 1.51 (d, J=6.5 Hz, 3H), 1.04 (s, 9H). HRMS m/z [M+H]⁺ calcd for C₄₇H₅₈N₉O₇S⁺ 892.4174, found 892.4202.

### Example 64

### Synthesis of LQ076-106

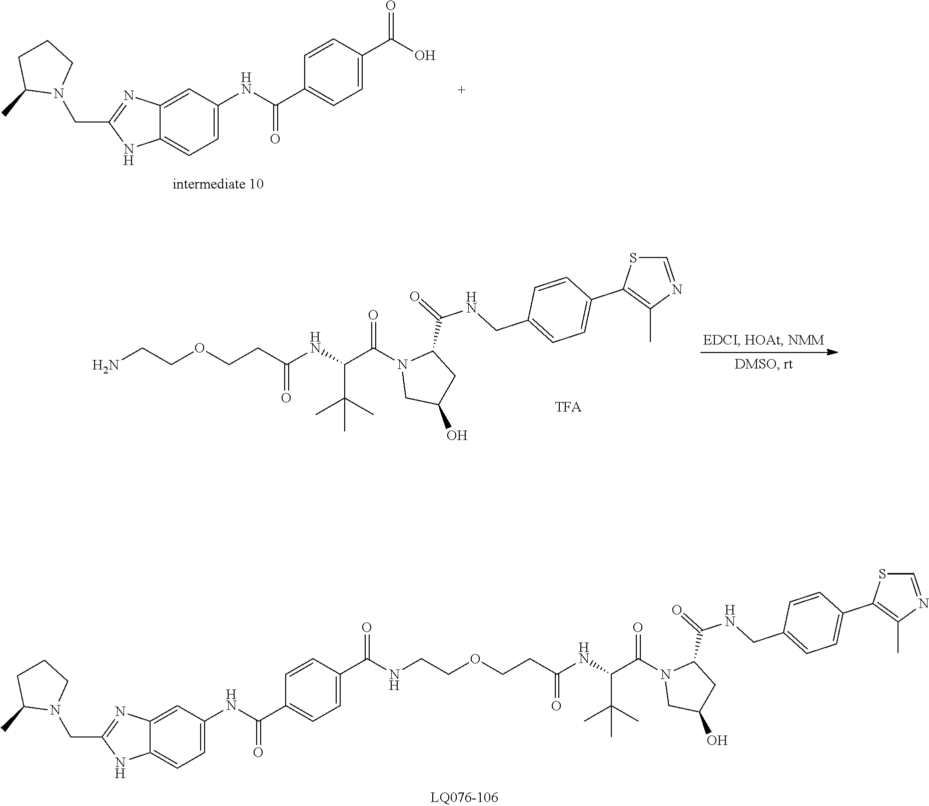 [SMILES: C[C@H]1CCCN1Cc1nc2cc(NC(=O)c3ccc(C(=O)O)cc3)ccc2[nH]1 ; Cc1ncsc1-c1ccc(CNC(=O)[C@@H]2C[C@@H](O)CN2C(=O)[C@@H](NC(=O)CCOCCN)C(C)(C)C)cc1 ; Cc1ncsc1-c1ccc(CNC(=O)[C@@H]2C[C@@H](O)CN2C(=O)[C@@H](NC(=O)CCOCCNC(=O)c2ccc(C(=O)Nc3ccc4[nH]c(CN5CCC[C@@H]5C)nc4c3)cc2)C(C)(C)C)cc1]

LQ076-106 was synthesized following the standard procedure for preparing LQ076-105 from intermediate 10 (10 mg, 0.02 mmol), (2S,4R)-1-((S)-2-(3-(2-aminoethoxy)propanamido)-3,3-dimethylbutanoyl)-4-hydroxy-N-(4-(4-methylthiazol-5-yl)benzyl)pyrrolidine-2-carboxamide (15.6 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-106 was obtained as white solid in TFA salt form (20 mg, 88%). ¹H NMR (600 MHz, Methanol-d₄) δ 9.13 (s, 1H), 8.35 (d, J=2.0 Hz, 1H), 8.06-7.97 (m, 4H), 7.72 (d, J=8.8 Hz, 1H), 7.63 (dd, J=8.8, 2.0 Hz, 1H), 7.48-7.39 (m, 4H), 4.90 (d, J=14.7 Hz, 1H), 4.68-4.57 (m, 3H), 4.54-4.49 (m, 2H), 4.37 (d, J=15.6 Hz, 1H), 3.91 (d, J=11.0 Hz, 1H), 3.83-3.57 (m, 11H), 3.50-3.43 (m, 1H), 2.63-2.52 (m, 2H), 2.48 (s, 3H), 2.44-2.36 (m, 1H), 2.28-2.23 (m, 1H), 2.19-2.06 (m, 3H), 1.88-1.80 (m, 1H), 1.52 (d, J=6.5 Hz, 3H), 1.02 (s, 9H). HRMS m/z [M+H]⁺ calcd for C₄₈H₆₀N₉O₇S⁺ 906.4331, found 906.4353.

### Example 65

### Synthesis of LQ076-107

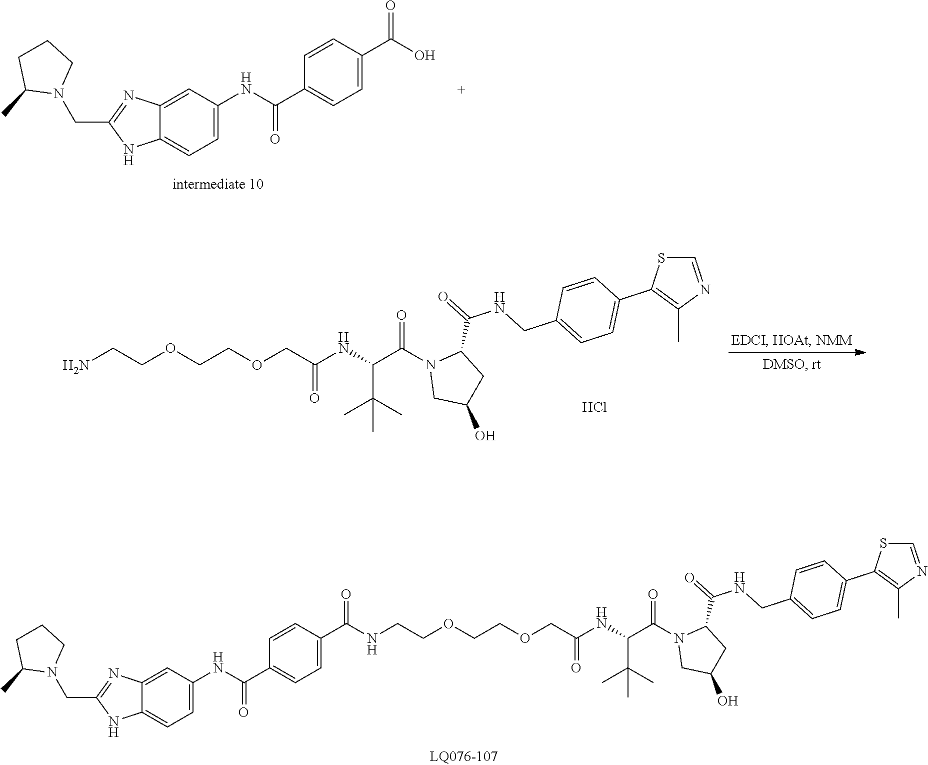 [SMILES: C[C@H]1CCCN1Cc1nc2cc(NC(=O)c3ccc(C(=O)O)cc3)ccc2[nH]1 ; Cc1ncsc1-c1ccc(CNC(=O)[C@@H]2C[C@@H](O)CN2C(=O)[C@@H](NC(=O)COCCOCCN)C(C)(C)C)cc1 ; Cl ; Cc1ncsc1-c1ccc(CNC(=O)[C@@H]2C[C@@H](O)CN2C(=O)[C@@H](NC(=O)COCCOCCNC(=O)c2ccc(C(=O)Nc3ccc4[nH]c(CN5CCC[C@@H]5C)nc4c3)cc2)C(C)(C)C)cc1]

LQ076-107 was synthesized following the standard procedure for preparing LQ076-105 from intermediate 10 (10 mg, 0.02 mmol), (2S,4R)-1-((S)-2-(2-(2-(2-aminoethoxy)ethoxy)acetamido)-3,3-dimethylbutanoyl)-4-hydroxy-N-(4-(4-methylthiazol-5-yl)benzyl)pyrrolidine-2-carboxamide (12.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-107 was obtained as white solid in TFA salt form (18.1 mg, 78%). ¹H NMR (600 MHz, Methanol-d₄) δ 9.06 (s, 1H), 8.32 (d, J=2.0 Hz, 1H), 8.07-7.93 (m, 4H), 7.70 (d, J=8.8 Hz, 1H), 7.59 (dd, J=8.7, 2.0 Hz, 1H), 7.47-7.38 (m, 4H), 4.85 (d, J=14.6 Hz, 1H), 4.78-4.72 (m, 1H), 4.64-4.48 (m, 4H), 4.39-4.31 (m, 1H), 4.08-3.98 (m, 2H), 3.92-3.81 (m, 2H), 3.78-3.55 (m, 11H), 3.50-3.44 (m, 1H), 2.48 (s, 3H), 2.43-2.37 (m, 1H), 2.30-2.24 (m, 1H), 2.20-2.07 (m, 3H), 1.87-1.79 (m, 1H), 1.51 (d, J=6.5 Hz, 3H), 1.05 (s, 9H). HRMS m/z [M+H]⁺ calcd for C₄₉H₆₂N₉O₈S⁺ 936.4437, found 936.4454.

### Example 66

### Synthesis of LQ076-108

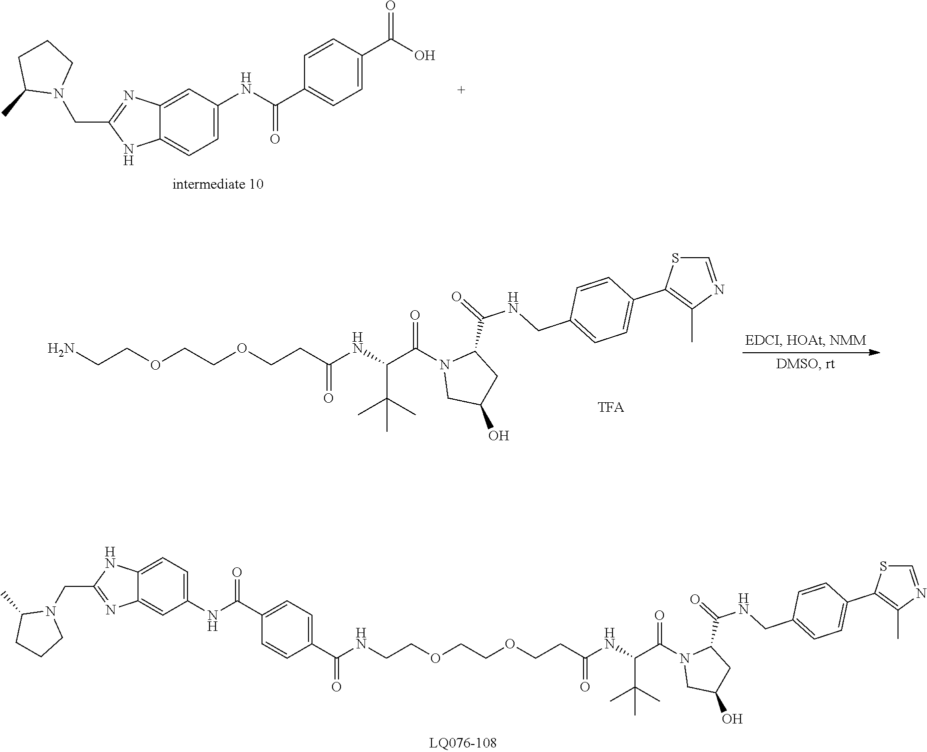 [SMILES: C[C@H]1CCCN1Cc1nc2cc(NC(=O)c3ccc(C(=O)O)cc3)ccc2[nH]1 ; Cc1ncsc1-c1ccc(CNC(=O)[C@@H]2C[C@@H](O)CN2C(=O)[C@@H](NC(=O)CCOCCOCCN)C(C)(C)C)cc1 ; Cc1ncsc1-c1ccc(CNC(=O)[C@@H]2C[C@@H](O)CN2C(=O)[C@@H](NC(=O)CCOCCOCCNC(=O)c2ccc(C(=O)Nc3ccc4[nH]c(CN5CCC[C@@H]5C)nc4c3)cc2)C(C)(C)C)cc1 ; "TFA"]

LQ076-108 was synthesized following the standard procedure for preparing LQ076-105 from intermediate 10 (10 mg, 0.02 mmol), (2S,4R)-1-((S)-2-(3-(2-(2-aminoethoxy)ethoxy)propanamido)-3,3-dimethylbutanoyl)-4-hydroxy-N-(4-(4-methylthiazol-5-yl)benzyl)pyrrolidine-2-carboxamide (16.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-108 was obtained as white solid in TFA salt form (19.7 mg, 83%). ¹H NMR (600 MHz, Methanol-d₄) δ 9.05 (s, 1H), 8.33 (d, J=2.0 Hz, 1H), 8.07-8.03 (m, 2H), 7.99-7.96 (m, 2H), 7.69 (d, J=8.8 Hz, 1H), 7.59 (dd, J=8.7, 2.0 Hz, 1H), 7.48-7.40 (m, 4H), 4.85 (d, J=14.6 Hz, 1H), 4.69-4.66 (m, 1H), 4.63-4.57 (m, 2H), 4.54-4.49 (m, 2H), 4.36 (d, J=15.5 Hz, 1H), 3.91 (d, J=11.0 Hz, 1H), 3.83-3.60 (m, 14H), 3.50-3.44 (m, 1H), 2.58-2.52 (m, 1H), 2.49 (s, 3H), 2.43-2.37 (m, 1H), 2.23 (d, J=13.1, 7.6 Hz, 1H), 2.18-2.06 (m, 3H), 1.87-1.79 (m, 1H), 1.51 (d, J=6.6 Hz, 3H), 1.05 (s, 9H). HRMS m/z [M+H]⁺ calcd for C₅₀H₆₄N₉O₈S⁺ 950.4593, found 950.4599.

### Example 67

### Synthesis of LQ076-109

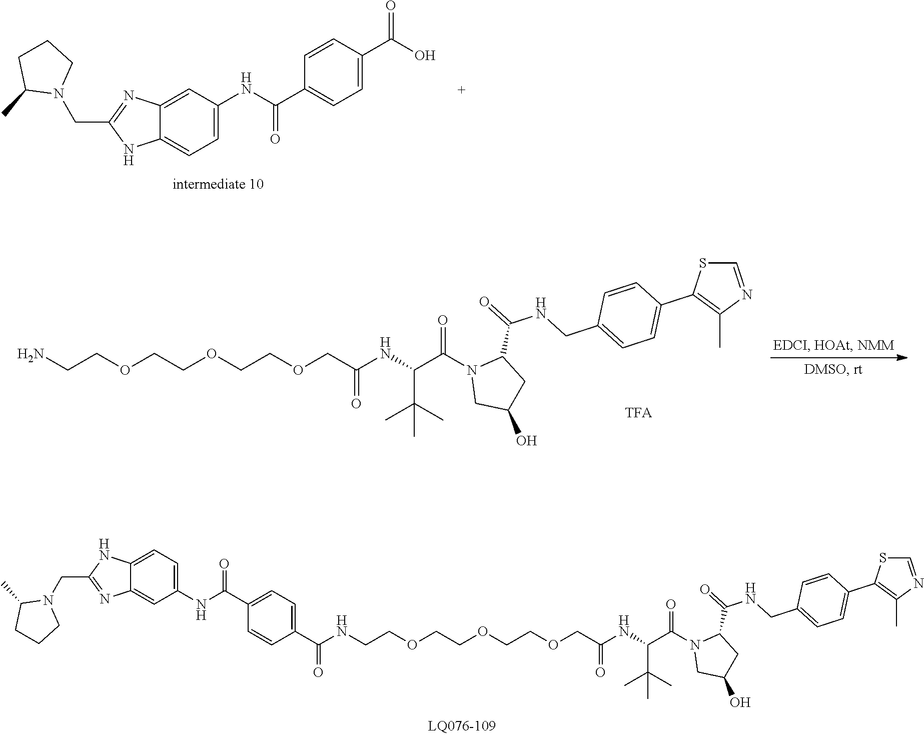

LQ076-109 was synthesized following the standard procedure for preparing LQ076-105 from intermediate 10 (10 mg, 0.02 mmol), (2S,4R)-1-((S)-14-amino-2-(tert-butyl)-4-oxo-6,9,12-trioxa-3-azatetradecanoyl)-4-hydroxy-N-(4-(4-methylthiazol-5-yl)benzyl)pyrrolidine-2-carboxamide (17.1 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-109 was obtained as white solid in TFA salt form (20.3 mg, 84%). ¹H NMR (600 MHz, Methanol-d₄) δ 9.08 (s, 1H), 8.34 (d, J=1.9 Hz, 1H), 8.06-8.02 (m, 2H), 7.99-7.95 (m, 2H), 7.70 (d, J=8.6 Hz, 1H), 7.60 (dd, J=8.7, 2.0 Hz, 1H), 7.49-7.42 (m, 4H), 4.86 (d, J=15.2 Hz, 1H), 4.74-4.70 (m, 1H), 4.65-4.49 (m, 4H), 4.36 (d, J=15.4 Hz, 1H), 4.06-3.94 (m, 2H), 3.90 (d, J=11.0 Hz, 1H), 3.84-3.79 (m, 1H), 3.78-3.56 (m, 14H), 3.50-3.44 (m, 1H), 2.50 (s, 3H), 2.44-2.36 (m, 1H), 2.28-2.23 (m, 1H), 2.19-2.06 (m, 3H), 1.87-1.79 (m, 1H), 1.51 (d, J=6.5 Hz, 3H), 1.05 (s, 9H). HRMS m/z [M+H]⁺ calcd for C₅₁H₆₆N₉O₉S⁺ 980.4699, found 980.4730.

### Example 68

### Synthesis of LQ076-110

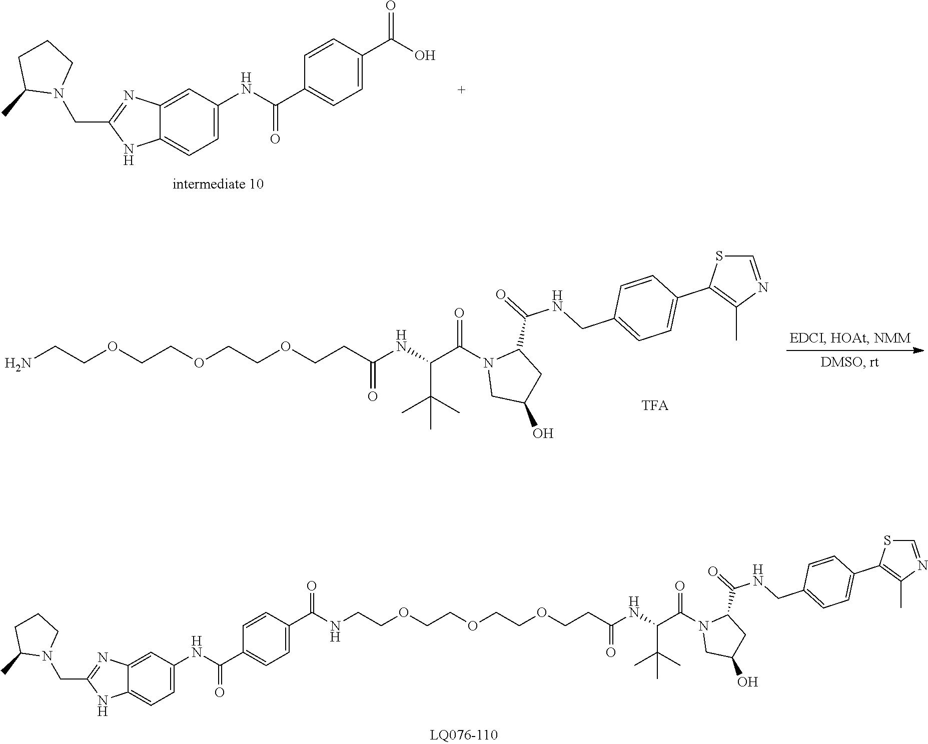

LQ076-110 was synthesized following the standard procedure for preparing LQ076-105 from intermediate 10 (10 mg, 0.02 mmol), (2S,4R)-1-((S)-1-amino-14-(tert-butyl)-12-oxo-3,6,9-trioxa-13-azapentadecan-15-oyl)-4-hydroxy-N-(4-(4-methylthiazol-5-yl)benzyl)pyrrolidine-2-carboxamide (17.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-110 was obtained as white solid in TFA salt form (19.1 mg, 78%). ¹H NMR (600 MHz, Methanol-d₄) δ 9.07 (s, 1H), 8.34 (d, J=1.9 Hz, 1H), 8.07-8.04 (m, 2H), 8.00-7.96 (m, 2H), 7.70 (d, J=8.8 Hz, 1H), 7.60 (dd, J=8.8, 2.0 Hz, 1H), 7.49-7.41 (m, 4H), 4.86 (d, J=14.7 Hz, 1H), 4.67-4.48 (m, 5H), 4.37 (d, J=15.6 Hz, 1H), 3.90 (d, J=11.0 Hz, 1H), 3.81-3.58 (m, 17H), 3.49-3.43 (m, 1H), 2.59-2.53 (m, 1H), 2.49 (s, 3H), 2.48-2.37 (m, 2H), 2.26-2.21 (m, 1H), 2.18-2.06 (m, 3H), 1.87-1.79 (m, 1H), 1.51 (d, J=6.5 Hz, 3H), 1.04 (s, 9H). HRMS m/z [M+H]⁺ calcd for C₅₂H₆₈N₉O₉S⁺ 994.4855, found 994.4898.

### Example 69

### Synthesis of LQ076-111

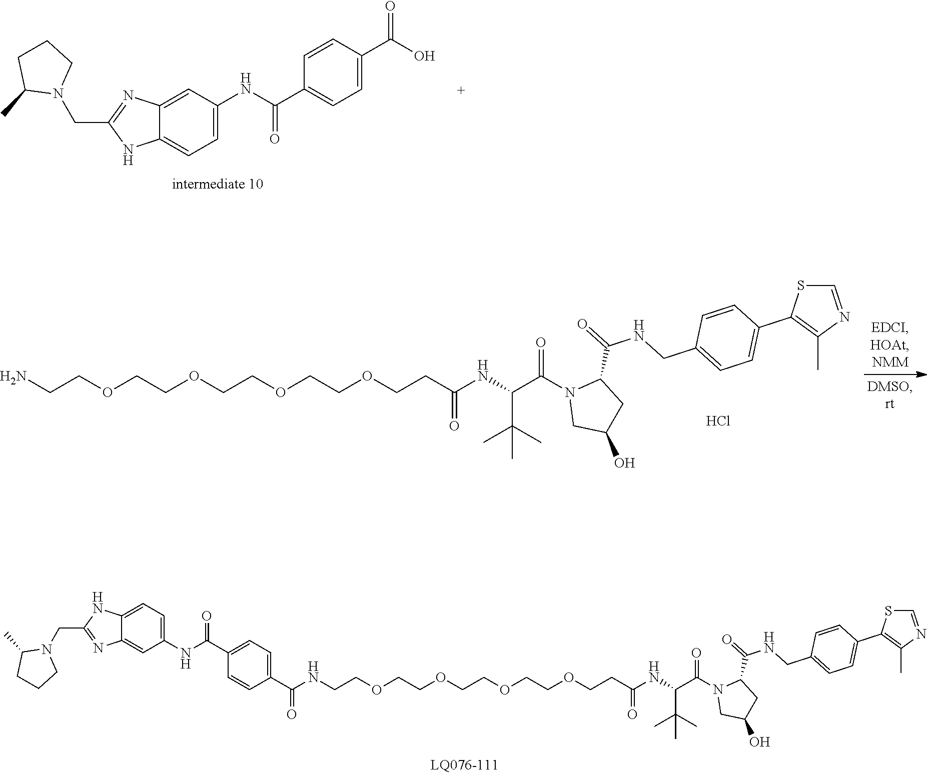 [SMILES: C[C@H]1CCCN1Cc1nc2cc(NC(=O)c3ccc(C(=O)O)cc3)ccc2[nH]1 ; Cc1ncsc1-c1ccc(CNC(=O)[C@@H]2C[C@@H](O)CN2C(=O)[C@@H](NC(=O)CCOCCOCCOCCOCCN)C(C)(C)C)cc1 ; Cl ; Cc1ncsc1-c1ccc(CNC(=O)[C@@H]2C[C@@H](O)CN2C(=O)[C@@H](NC(=O)CCOCCOCCOCCOCCNC(=O)c2ccc(C(=O)Nc3ccc4[nH]c(CN5CCC[C@@H]5C)nc4c3)cc2)C(C)(C)C)cc1]

LQ076-111 was synthesized following the standard procedure for preparing LQ076-105 from intermediate 10 (10 mg, 0.02 mmol), (2S,4R)-1-((S)-1-amino-17-(tert-butyl)-15-oxo-3,6,9,12-tetraoxa-16-azaoctadecan-18-oyl)-4-hydroxy-N-(4-(4-methylthiazol-5-yl)benzyl)pyrrolidine-2-carboxamide (14.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-111 was obtained as white solid in TFA salt form (18.2 mg, 72%). ¹H NMR (600 MHz, Methanol-d₄) δ 9.02 (s, 1H), 8.32 (d, J=2.0 Hz, 1H), 8.08-8.04 (m, 2H), 8.01-7.98 (m, 2H), 7.69 (d, J=8.8 Hz, 1H), 7.57 (dd, J=8.8, 2.0 Hz, 1H), 7.49-7.41 (m, 4H), 4.83 (d, J=14.7 Hz, 1H), 4.67-4.49 (m, 5H), 4.37 (d, J=15.6 Hz, 1H), 3.90 (d, J=11.0 Hz, 1H), 3.82-3.58 (m, 21H), 3.50-3.43 (m, 1H), 2.60-2.54 (m, 1H), 2.49 (s, 3H), 2.48-2.36 (m, 2H), 2.26-2.21 (m, 1H), 2.19-2.05 (m, 3H), 1.86-1.79 (m, 1H), 1.51 (d, J=6.5 Hz, 3H), 1.04 (s, 9H). HRMS m/z [M+H]⁺ calcd for C₅₄H₇₂N₉O₁₀S⁺ 1038.5117, found 1038.55152.

### Example 70

### Synthesis of LQ076-112

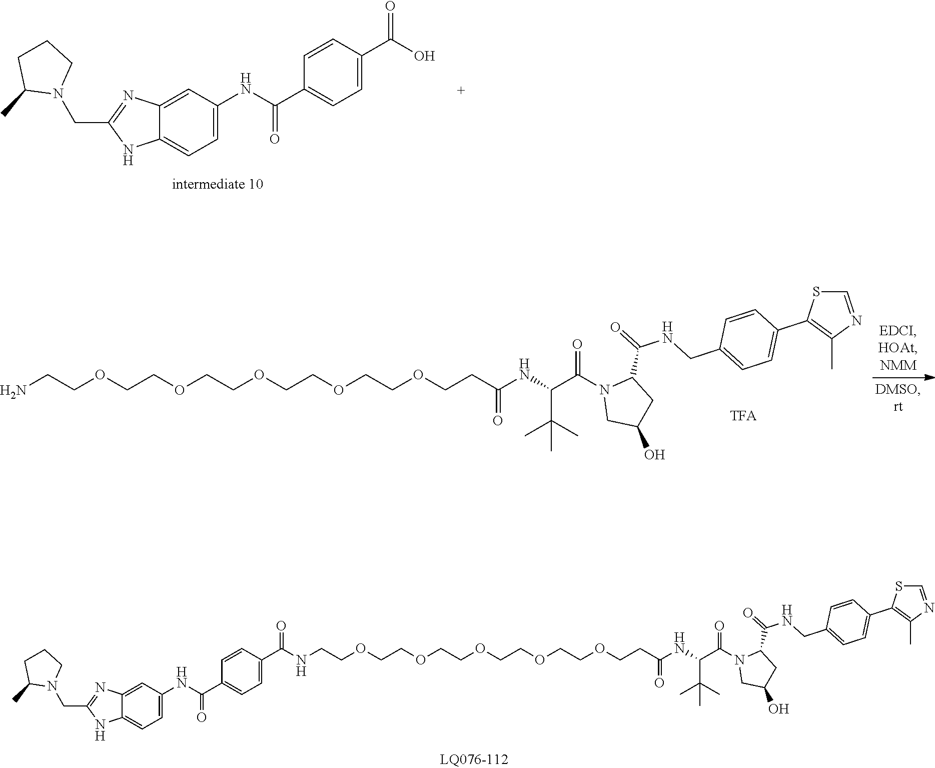

LQ076-112 was synthesized following the standard procedure for preparing LQ076-105 from intermediate 10 (10 mg, 0.02 mmol), (2S,4R)-1-((S)-1-amino-20-(tert-butyl)-18-oxo-3,6,9,12,15-pentaoxa-19-azahenicosan-21-oyl)-4-hydroxy-N-(4-(4-methylthiazol-5-yl)benzyl)pyrrolidine-2-carboxamide (19.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-112 was obtained as white solid in TFA salt form (20.3 mg, 77%). ¹H NMR (600 MHz, Methanol-d₄) δ 9.08 (s, 1H), 8.34 (d, J=2.0 Hz, 1H), 8.08-8.05 (m, 2H), 8.01-7.98 (m, 2H), 7.70 (d, J=8.8 Hz, 1H), 7.60 (dd, J=8.8, 2.0 Hz, 1H), 7.50-7.42 (m, 4H), 4.85 (d, J=14.6 Hz, 1H), 4.67-4.49 (m, 5H), 4.37 (d, J=15.5 Hz, 1H), 3.90 (d, J=11.0 Hz, 1H), 3.83-3.57 (m, 25H), 3.50-3.43 (m, 1H), 2.60-2.54 (m, 1H), 2.50 (s, 3H), 2.49-2.37 (m, 2H), 2.26-2.21 (m, 1H), 2.18-2.06 (m, 3H), 1.86-1.79 (m, 1H), 1.51 (d, J=6.5 Hz, 3H), 1.04 (s, 9H). HRMS m/z [M+H]⁺ calcd for C₅₆H₇₆N₉O₁₁S⁺ 1082.5380, found 1082.5399.

### Example 71

### Synthesis of LQ076-113

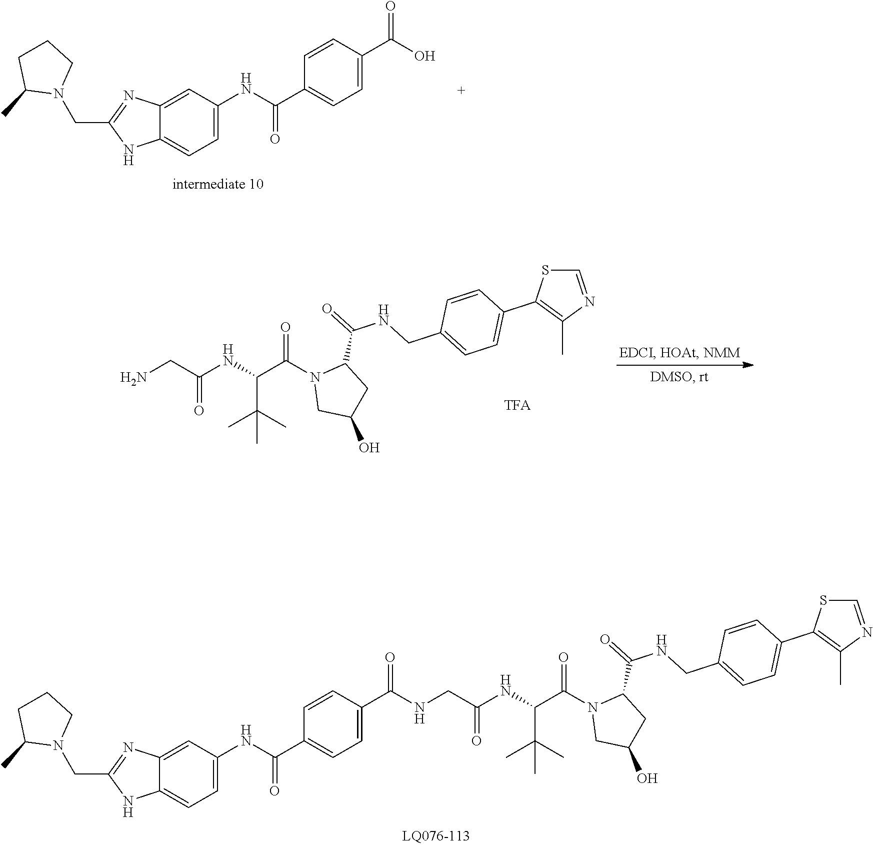

LQ076-113 was synthesized following the standard procedure for preparing LQ076-105 from intermediate 10 (10 mg, 0.02 mmol), (2S,4R)-1-((S)-2-(2-aminoacetamido)-3,3-dimethylbutanoyl)-4-hydroxy-N-(4-(4-methylthiazol-5-yl)benzyl)pyrrolidine-2-carboxamide (14.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-113 was obtained as white solid in TFA salt form (17.6 mg, 82%). ¹H NMR (600 MHz, Methanol-d₄) δ 9.07 (s, 1H), 8.33 (d, J=2.0 Hz, 1H), 8.08-8.00 (m, 4H), 7.70 (d, J=8.8 Hz, 1H), 7.59 (dd, J=8.8, 2.0 Hz, 1H), 7.51-7.48 (m, 2H), 7.45-7.41 (m, 2H), 4.85 (d, J=14.6 Hz, 1H), 4.72-4.69 (m, 1H), 4.65-4.50 (m, 4H), 4.37 (d, J=15.5 Hz, 1H), 4.20-4.10 (m, 2H), 3.93 (d, J=11.0 Hz, 1H), 3.83 (dd, J=10.9, 3.8 Hz, 1H), 3.79-3.71 (m, 2H), 3.50-3.44 (m, 1H), 2.50 (s, 3H), 2.44-2.37 (m, 1H), 2.28-2.22 (m, 1H), 2.19-2.07 (m, 3H), 1.87-1.79 (m, 1H), 1.52 (d, J=6.5 Hz, 3H), 1.07 (s, 9H). HRMS m/z [M+H]⁺ calcd for C₄₅H₅₄N₉O₆S⁺ 848.3912, found 848.3970.

### Example 72

### Synthesis of LQ076-114

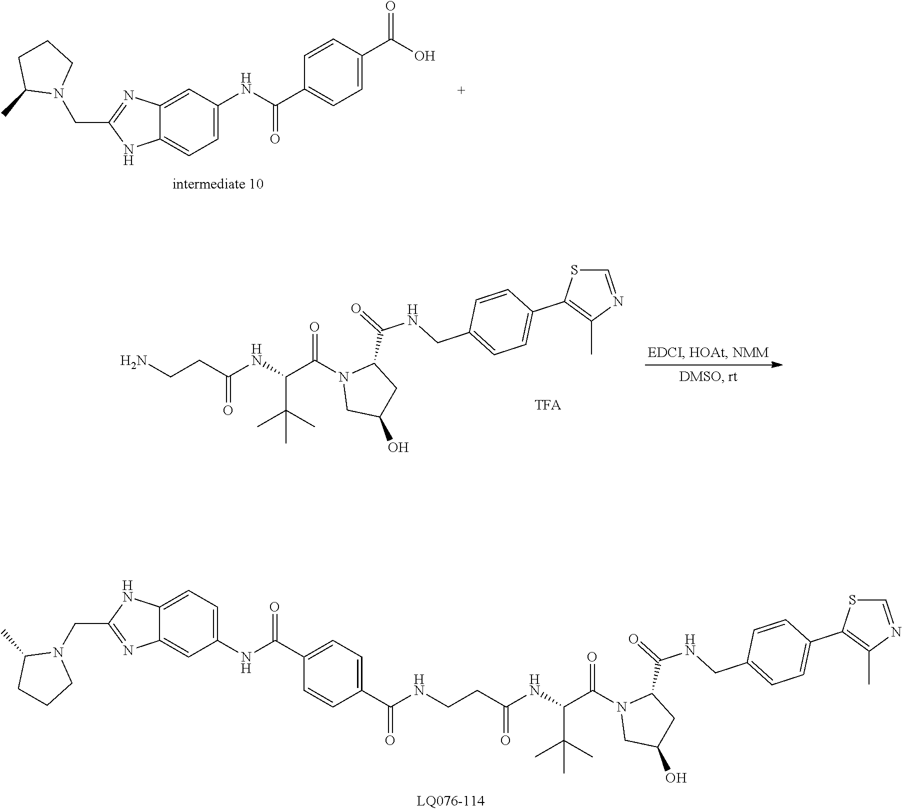 [SMILES: C[C@H]1CCCN1Cc1nc2cc(NC(=O)c3ccc(C(=O)O)cc3)ccc2[nH]1 ; Cc1ncsc1-c1ccc(CNC(=O)[C@@H]2C[C@@H](O)CN2C(=O)[C@@H](NC(=O)CCN)C(C)(C)C)cc1 ; Cc1ncsc1-c1ccc(CNC(=O)[C@@H]2C[C@@H](O)CN2C(=O)[C@@H](NC(=O)CCNC(=O)c2ccc(C(=O)Nc3ccc4[nH]c(CN5CCC[C@@H]5C)nc4c3)cc2)C(C)(C)C)cc1 ; "TFA"]

LQ076-114 was synthesized following the standard procedure for preparing LQ076-105 from intermediate 10 (10 mg, 0.02 mmol), (2S,4R)-1-((S)-2-(3-aminopropanamido)-3,3-dimethylbutanoyl)-4-hydroxy-N-(4-(4-methylthiazol-5-yl)benzyl)pyrrolidine-2-carboxamide (14.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-114 was obtained as white solid in TFA salt form (16.3 mg, 84%). ¹H NMR (600 MHz, Methanol-d₄) δ 9.05 (s, 1H), 8.31 (d, J=2.0 Hz, 1H), 8.05-8.01 (m, 2H), 7.99-7.95 (m, 2H), 7.69 (d, J=8.8 Hz, 1H), 7.57 (dd, J=8.7, 2.0 Hz, 1H), 7.50-7.47 (m, 2H), 7.44-7.40 (m, 2H), 4.84 (d, J=14.6 Hz, 1H), 4.68-4.65 (m, 1H), 4.63-4.51 (m, 4H), 4.38 (d, J=15.5 Hz, 1H), 3.97 (d, J=11.0 Hz, 1H), 3.82 (dd, J=11.0, 3.9 Hz, 1H), 3.78-3.71 (m, 3H), 3.68-3.61 (m, 1H), 3.49-3.44 (m, 1H), 2.69-2.61 (m, 2H), 2.48 (s, 3H), 2.43-2.37 (m, 1H), 2.28-2.22 (m, 1H), 2.19-2.06 (m, 3H), 1.88-1.79 (m, 1H), 1.51 (d, J=6.5 Hz, 3H), 1.05 (s, 9H). HRMS m/z [M+H]⁺ calcd for C₄₆H₅₆N₉O₆S⁺ 862.4069, found 862.4082.

### Example 73

### Synthesis of LQ076-115

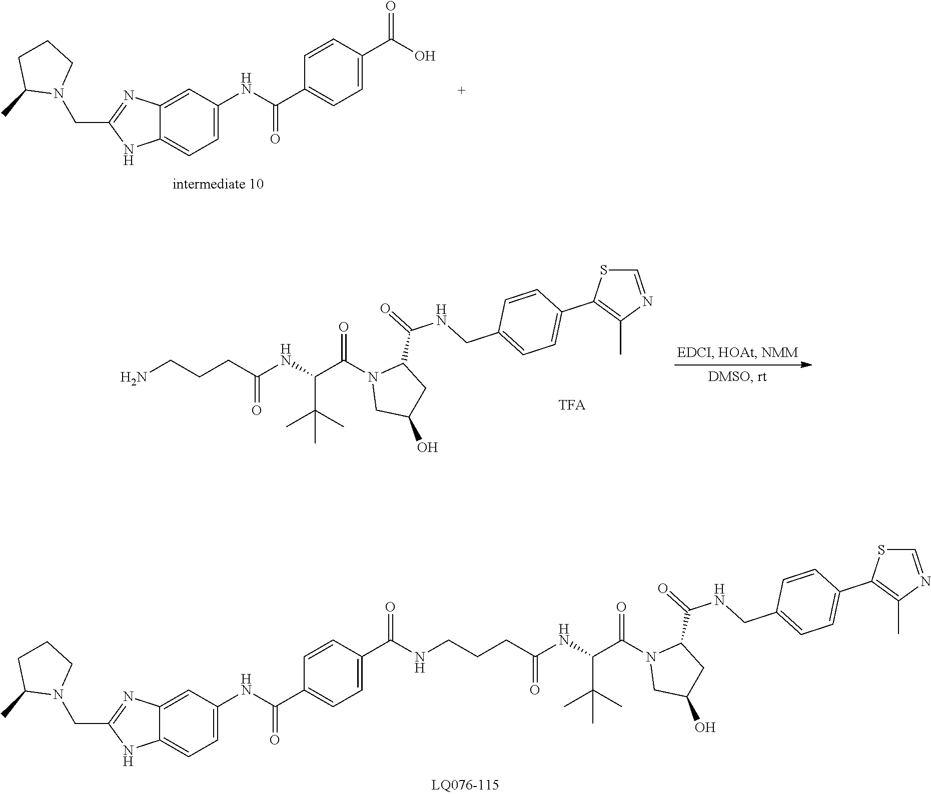

LQ076-115 was synthesized following the standard procedure for preparing LQ076-105 from intermediate 10 (10 mg, 0.02 mmol), (2S,4R)-1-((S)-2-(4-aminobutanamido)-3,3-dimethylbutanoyl)-4-hydroxy-N-(4-(4-methylthiazol-5-yl)benzyl)pyrrolidine-2-carboxamide (15.1 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-115 was obtained as white solid in TFA salt form (15.3 mg, 69%). ¹H NMR (600 MHz, Methanol-d₄) δ 9.07 (s, 1H), 8.33 (d, J=1.9 Hz, 1H), 8.07-8.04 (m, 2H), 8.00-7.97 (m, 2H), 7.70 (d, J=8.8 Hz, 1H), 7.59 (dd, J=8.8, 2.0 Hz, 1H), 7.51-7.48 (m, 2H), 7.46-7.42 (m, 2H), 4.85 (d, J=14.7 Hz, 1H), 4.66-4.51 (m, 5H), 4.38 (d, J=15.5 Hz, 1H), 3.95 (d, J=11.0 Hz, 1H), 3.83 (dd, J=10.9, 3.9 Hz, 1H), 3.78-3.71 (m, 2H), 3.50-3.41 (m, 3H), 2.50 (s, 3H), 2.44-2.36 (m, 3H), 2.27-2.22 (m, 1H), 2.17-2.07 (m, 3H), 1.98-1.91 (m, 2H), 1.87-1.79 (m, 1H), 1.51 (d, J=6.6 Hz, 3H), 1.07 (s, 9H). HRMS m/z [M+H]⁺ calcd for C₄₇H₅₈N₉O₆S⁺ 876.4225, found 876.4252.

### Example 74

### Synthesis of LQ076-116

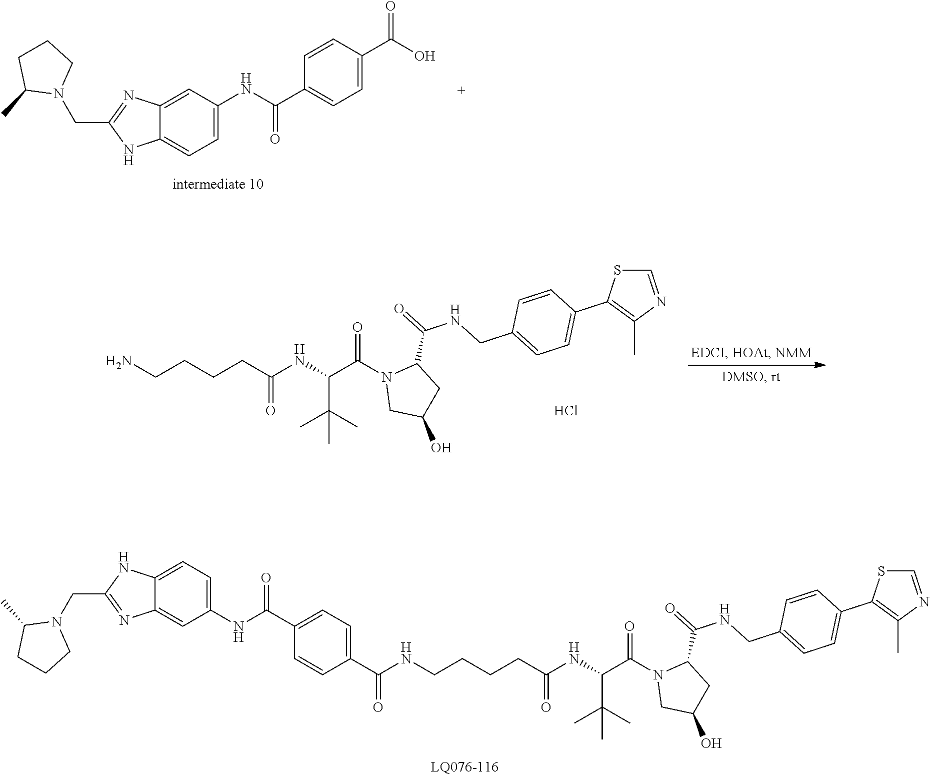

LQ076-116 was synthesized following the standard procedure for preparing LQ076-105 from intermediate 10 (10 mg, 0.02 mmol), (2S,4R)-1-((S)-2-(5-aminopentanamido)-3,3-dimethylbutanoyl)-4-hydroxy-N-(4-(4-methylthiazol-5-yl)benzyl)pyrrolidine-2-carboxamide (11.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-116 was obtained as white solid in TFA salt form (15.7 mg, 70%). ¹H NMR (600 MHz, Methanol-d₄) δ 9.04 (s, 1H), 8.31 (d, J=1.9 Hz, 1H), 8.05 (d, J=8.3 Hz, 2H), 7.98 (d, J=8.3 Hz, 2H), 7.69 (d, J=8.8 Hz, 1H), 7.57 (dd, J=8.7, 2.0 Hz, 1H), 7.49 (s, 2H), 7.46-7.42 (m, 2H), 4.82 (d, J=14.6 Hz, 1H), 4.66-4.64 (m, 1H), 4.61-4.51 (m, 4H), 4.38 (d, J=15.5 Hz, 1H), 3.93 (d, J=11.0 Hz, 1H), 3.83 (dd, J=11.0, 4.0 Hz, 1H), 3.78-3.71 (m, 2H), 3.49-3.41 (m, 3H), 2.50 (s, 3H), 2.43-2.33 (m, 3H), 2.26-2.21 (m, 1H), 2.17-2.07 (m, 3H), 1.86-1.79 (m, 1H), 1.75-1.66 (m, 4H), 1.51 (d, J=6.6 Hz, 3H), 1.06 (s, 9H). HRMS m/z [M+H]⁺ calcd for C₄₈H₆₀N₉O₆S⁺ 890.4382, found 890.4414.

### Example 75

### Synthesis of LQ076-117

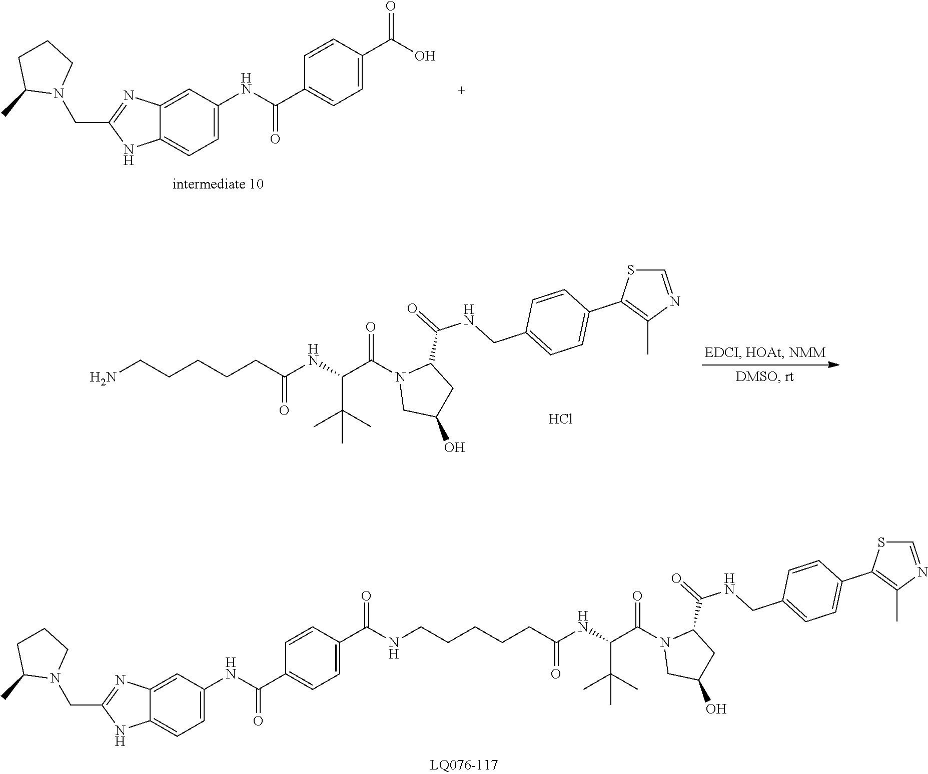

LQ076-117 was synthesized following the standard procedure for preparing LQ076-105 from intermediate 10 (10 mg, 0.02 mmol), (2S,4R)-1-((S)-2-(6-aminohexanamido)-3,3-dimethylbutanoyl)-4-hydroxy-N-(4-(4-methylthiazol-5-yl)benzyl)pyrrolidine-2-carboxamide (12 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-117 was obtained as white solid in TFA salt form (14.9 mg, 66%). ¹H NMR (600 MHz, Methanol-d₄) δ 9.02 (s, 1H), 8.31 (d, J=1.9 Hz, 1H), 8.05 (d, J=8.2 Hz, 2H), 7.97 (d, J=8.4 Hz, 2H), 7.69 (d, J=8.7 Hz, 1H), 7.57 (dd, J=8.7, 2.0 Hz, 1H), 7.51-7.47 (m, 2H), 7.45-7.42 (m, 2H), 4.82 (d, J=14.7 Hz, 1H), 4.67-4.64 (m, 1H), 4.62-4.51 (m, 4H), 4.37 (d, J=15.6 Hz, 1H), 3.93 (d, J=11.0 Hz, 1H), 3.82 (dd, J=10.9, 3.9 Hz, 1H), 3.78-3.70 (m, 2H), 3.50-3.41 (m, 3H), 2.50 (s, 3H), 2.43-2.21 (m, 4H), 2.18-2.07 (m, 3H), 1.86-1.79 (m, 1H), 1.73-1.64 (m, 4H), 1.51 (d, J=6.5 Hz, 3H), 1.48-1.42 (m, 2H), 1.05 (s, 9H). HRMS m/z [M+H]⁺ calcd for C₄₉H₆₂N₉O₆S⁺ 904.4538, found 904.4587.

### Example 76

### Synthesis of LQ076-118

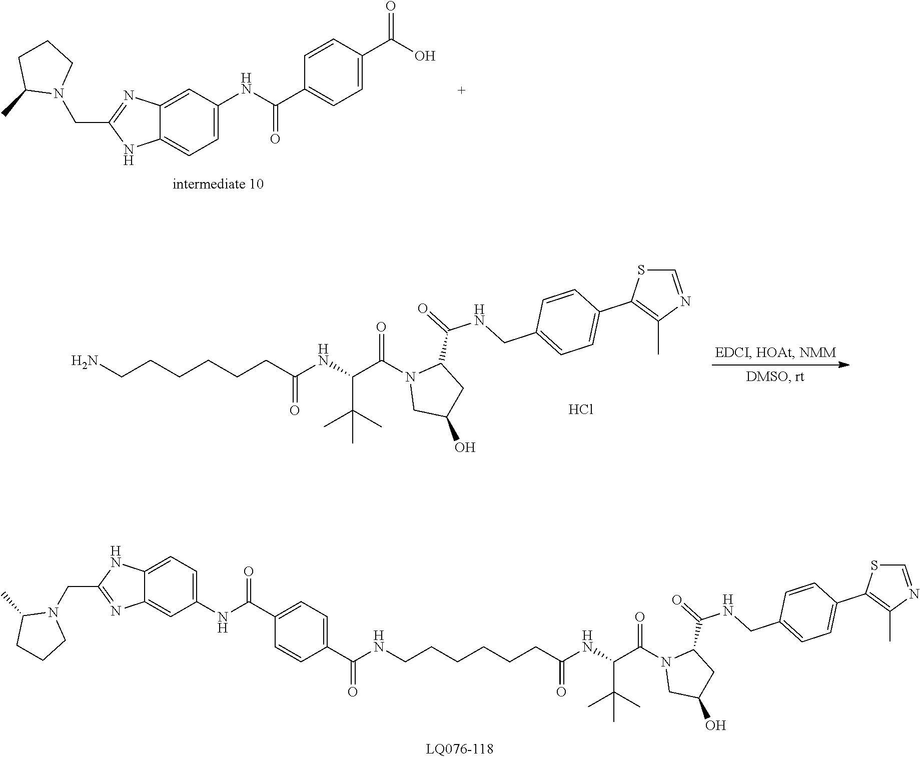

LQ076-118 was synthesized following the standard procedure for preparing LQ076-105 from intermediate 10 (10 mg, 0.02 mmol), (2S,4R)-1-((S)-2-(7-aminoheptanamido)-3,3-dimethylbutanoyl)-4-hydroxy-N-(4-(4-methylthiazol-5-yl)benzyl)pyrrolidine-2-carboxamide (12.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-118 was obtained as white solid in TFA salt form (15.4 mg, 67%). ¹H NMR (600 MHz, Methanol-d₄) δ 9.01 (s, 1H), 8.31 (d, J=2.0 Hz, 1H), 8.05 (d, J=8.2 Hz, 2H), 7.96 (d, J=8.4 Hz, 2H), 7.68 (d, J=8.7 Hz, 1H), 7.56 (dd, J=8.8, 2.0 Hz, 1H), 7.50-7.47 (m, 2H), 7.46-7.42 (m, 2H), 4.82 (d, J=14.7 Hz, 1H), 4.67-4.65 (m, 1H), 4.63-4.50 (m, 4H), 4.38 (d, J=15.5 Hz, 1H), 3.93 (d, J=11.1 Hz, 1H), 3.82 (dd, J=11.0, 3.9 Hz, 1H), 3.77-3.70 (m, 2H), 3.49-3.40 (m, 3H), 2.50 (s, 3H), 2.43-2.27 (m, 3H), 2.26-2.21 (m, 1H), 2.18-2.07 (m, 3H), 1.86-1.79 (m, 1H), 1.69-1.64 (m, 4H), 1.51 (d, J=6.5 Hz, 3H), 1.46-1.39 (m, 4H), 1.06 (s, 9H). HRMS m/z [M+H]⁺ calcd for C₅₀H₆₄N₉O₆S⁺ 918.4695, found 918.4592.

### Example 77

### Synthesis of LQ076-119

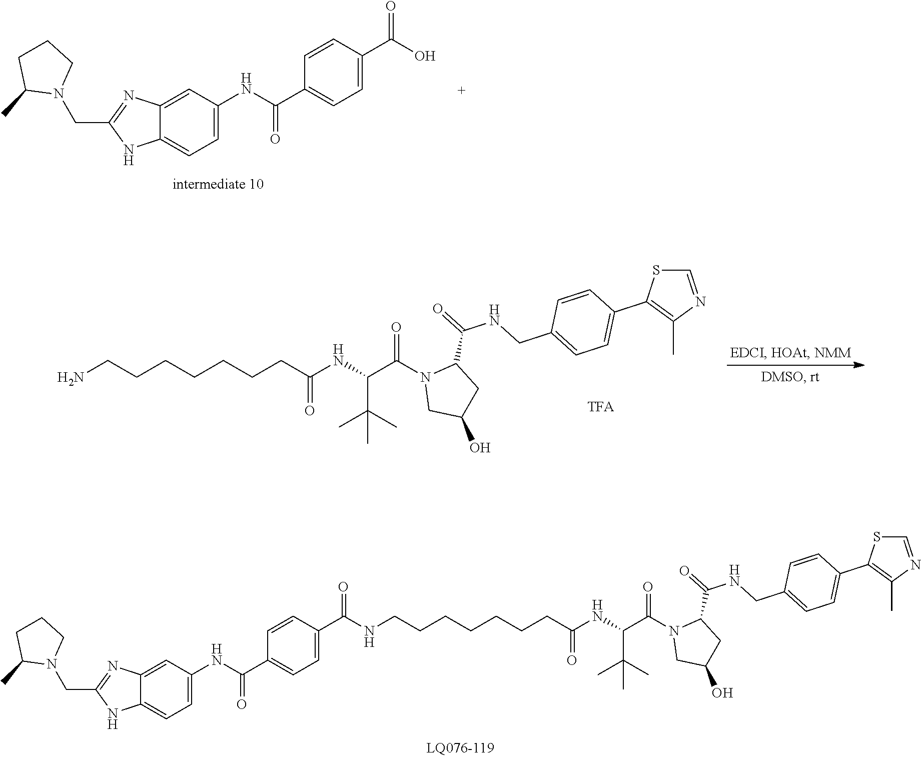

LQ076-119 was synthesized following the standard procedure for preparing LQ076-105 from intermediate 10 (10 mg, 0.02 mmol), (2S,4R)-1-((S)-2-(8-aminooctanamido)-3,3-dimethylbutanoyl)-4-hydroxy-N-(4-(4-methylthiazol-5-yl)benzyl)pyrrolidine-2-carboxamide (16.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-119 was obtained as white solid in TFA salt form (17.3 mg, 75%). ¹H NMR (600 MHz, Methanol-d₄) δ 8.97 (s, 1H), 8.30 (d, J=2.0 Hz, 1H), 8.04 (d, J=8.1 Hz, 2H), 7.96 (d, J=8.2 Hz, 2H), 7.68 (d, J=8.7 Hz, 1H), 7.56 (dd, J=8.8, 2.0 Hz, 1H), 7.50-7.46 (m, 2H), 7.45-7.41 (m, 2H), 4.81 (d, J=14.6 Hz, 1H), 4.67-4.65 (m, 1H), 4.62-4.50 (m, 4H), 4.37 (d, J=15.5 Hz, 1H), 3.92 (d, J=11.0 Hz, 1H), 3.82 (dd, J=10.9, 3.9 Hz, 1H), 3.77-3.71 (m, 2H), 3.50-3.39 (m, 3H), 2.49 (s, 3H), 2.43-2.20 (m, 4H), 2.18-2.06 (m, 3H), 1.87-1.78 (m, 1H), 1.69-1.61 (m, 4H), 1.51 (d, J=6.5 Hz, 3H), 1.45-1.35 (m, 6H), 1.05 (s, 9H). HRMS m/z [M+H]⁺ calcd for C₅₁H₆₆N₉O₆S⁺ 932.4851, found 932.4872.

### Example 78

### Synthesis of LQ076-120

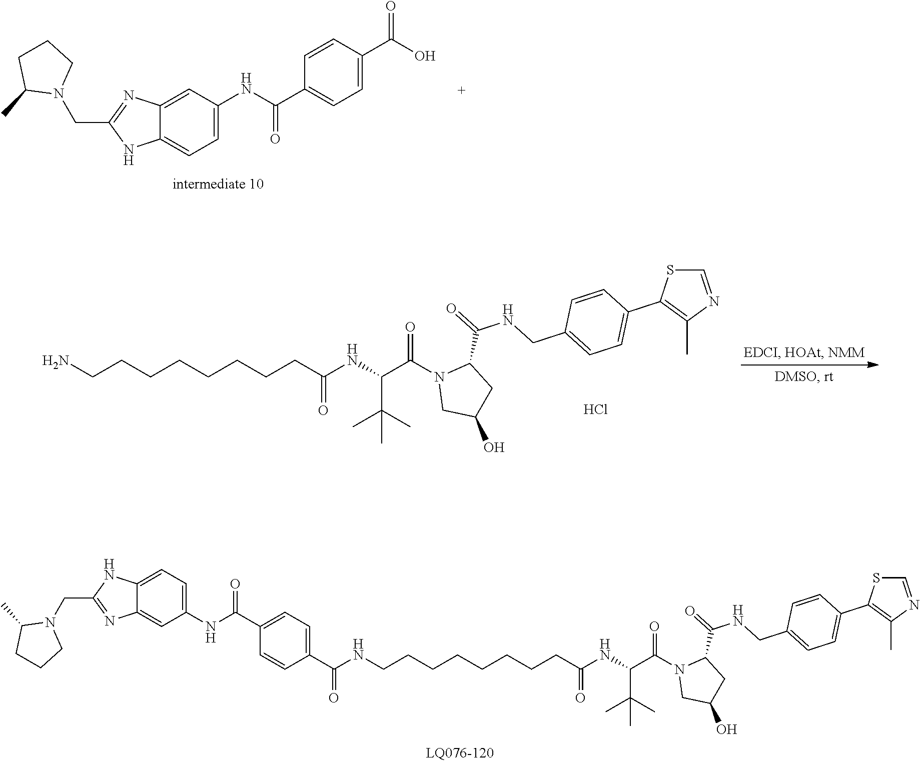 [SMILES: C[C@H]1CCCN1Cc1nc2cc(NC(=O)c3ccc(C(=O)O)cc3)ccc2[nH]1 ; Cc1ncsc1-c1ccc(CNC(=O)[C@@H]2C[C@@H](O)CN2C(=O)[C@@H](NC(=O)CCCCCCCCN)C(C)(C)C)cc1 ; Cl ; Cc1ncsc1-c1ccc(CNC(=O)[C@@H]2C[C@@H](O)CN2C(=O)[C@@H](NC(=O)CCCCCCCCNC(=O)c2ccc(C(=O)Nc3ccc4[nH]c(CN5CCC[C@@H]5C)nc4c3)cc2)C(C)(C)C)cc1]

LQ076-120 was synthesized following the standard procedure for preparing LQ076-105 from intermediate 10 (10 mg, 0.02 mmol), (2S,4R)-1-((S)-2-(9-aminononanamido)-3,3-dimethylbutanoyl)-4-hydroxy-N-(4-(4-methylthiazol-5-yl)benzyl)pyrrolidine-2-carboxamide (13.7 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-120 was obtained as white solid in TFA salt form (17.7 mg, 75%). ¹H NMR (600 MHz, Methanol-d₄) δ 9.03 (s, 1H), 8.31 (d, J=1.9 Hz, 1H), 8.05 (d, J=8.3 Hz, 2H), 7.96 (d, J=8.3 Hz, 2H), 7.69 (d, J=8.7 Hz, 1H), 7.58 (dd, J=8.7, 2.0 Hz, 1H), 7.50-7.47 (m, 2H), 7.45-7.42 (m, 2H), 4.83 (d, J=14.6 Hz, 1H), 4.67-4.64 (m, 1H), 4.62-4.50 (m, 4H), 4.38 (d, J=15.5 Hz, 1H), 3.92 (d, J=11.0 Hz, 1H), 3.82 (dd, J=11.0, 3.9 Hz, 1H), 3.77-3.71 (m, 2H), 3.50-3.40 (m, 3H), 2.50 (s, 3H), 2.43-2.37 (m, 1H), 2.34-2.21 (m, 3H), 2.18-2.07 (m, 3H), 1.86-1.79 (m, 1H), 1.68-1.60 (m, 4H), 1.51 (d, J=6.5 Hz, 3H), 1.44-1.33 (m, 8H), 1.05 (s, 9H). HRMS m/z [M+H]⁺ calcd for C₅₂H₆₈N₉O₆S⁺ 946.5008, found 946.4933.

### Example 79

### Synthesis of LQ076-121

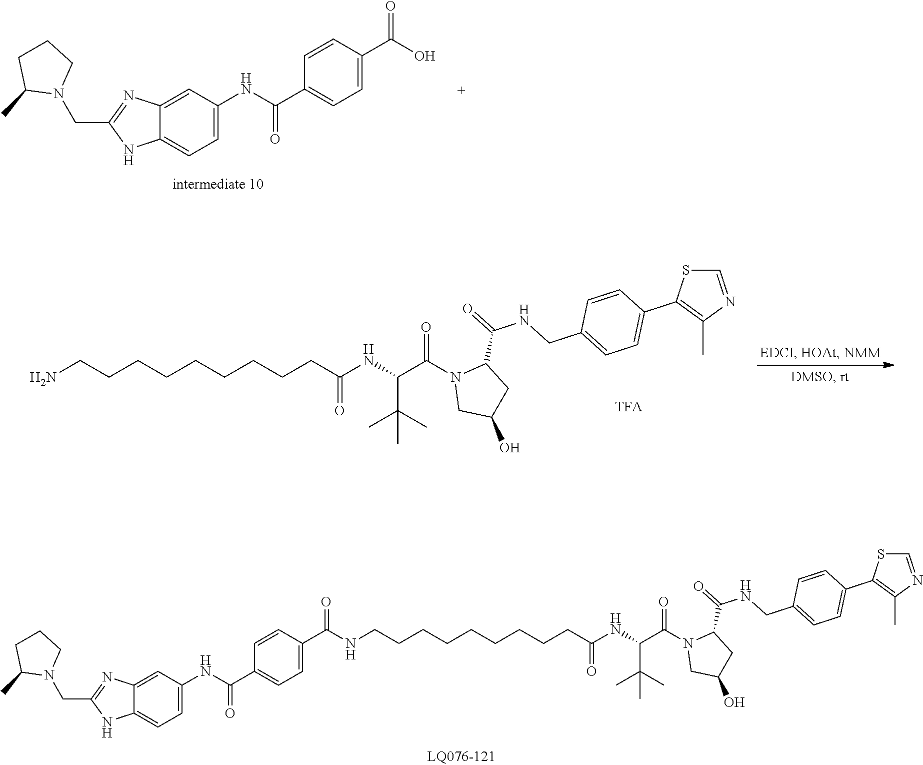

LQ076-121 was synthesized following the standard procedure for preparing LQ076-105 from intermediate 10 (10 mg, 0.02 mmol), (2S,4R)-1-((S)-2-(10-aminodecanamido)-3,3-dimethylbutanoyl)-4-hydroxy-N-(4-(4-methylthiazol-5-yl)benzyl)pyrrolidine-2-carboxamide (17.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-121 was obtained as white solid in TFA salt form (16.3 mg, 69%). ¹H NMR (800 MHz, Methanol-d₄) δ 8.98 (s, 1H), 8.30 (s, 1H), 8.05 (d, J=8.0 Hz, 2H), 7.97 (d, J=7.9 Hz, 2H), 7.69 (d, J=8.7 Hz, 1H), 7.58 (d, J=8.7 Hz, 1H), 7.50-7.47 (m, 2H), 7.45-7.41 (m, 2H), 4.82 (d, J=14.6 Hz, 1H), 4.67-4.65 (m, 1H), 4.62-4.50 (m, 4H), 4.38 (d, J=15.4 Hz, 1H), 3.93 (d, J=11.0 Hz, 1H), 3.82 (dd, J=10.9, 3.9 Hz, 1H), 3.78-3.71 (m, 2H), 3.49-3.44 (m, 1H), 3.44-3.38 (m, 2H), 2.50 (s, 3H), 2.43-2.37 (m, 1H), 2.34-2.29 (m, 1H), 2.28-2.22 (m, 2H), 2.18-2.07 (m, 3H), 1.86-1.80 (m, 1H), 1.68-1.59 (m, 4H), 1.51 (d, J=6.6 Hz, 3H), 1.44-1.35 (m, 10H), 1.06 (s, 9H). HRMS m/z [M+H]⁺ calcd for C₅₃H₇₀N₉O₆S⁺ 960.5164, found 960.5074.

### Example 80

### Synthesis of LQ076-122

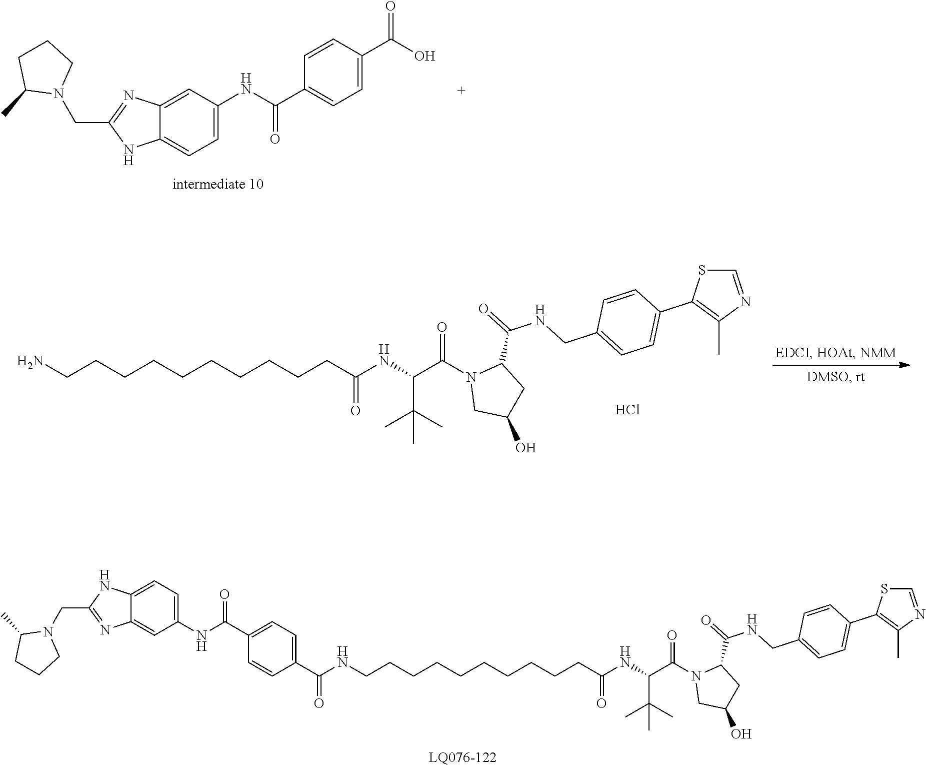

LQ076-122 was synthesized following the standard procedure for preparing LQ076-105 from intermediate 10 (10 mg, 0.02 mmol), (2S,4R)-1-((S)-2-(11-aminoundecanamido)-3,3-dimethylbutanoyl)-4-hydroxy-N-(4-(4-methylthiazol-5-yl)benzyl)pyrrolidine-2-carboxamide (14.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-122 was obtained as white solid in TFA salt form (16.7 mg, 69%). ¹H NMR (600 MHz, Methanol-d₄) δ 8.97 (s, 1H), 8.30 (d, J=1.7 Hz, 1H), 8.05 (d, J=8.1 Hz, 2H), 7.97 (d, J=8.3 Hz, 2H), 7.68 (d, J=8.7 Hz, 1H), 7.56 (dd, J=8.7, 2.0 Hz, 1H), 7.50-7.46 (m, 2H), 7.45-7.42 (m, 2H), 4.80 (d, J=14.6 Hz, 1H), 4.66-4.64 (m, 1H), 4.61-4.50 (m, 4H), 4.37 (d, J=15.5 Hz, 1H), 3.92 (d, J=11.0 Hz, 1H), 3.81 (dd, J=11.0, 3.9 Hz, 1H), 3.77-3.71 (m, 2H), 3.49-3.39 (m, 3H), 2.49 (s, 3H), 2.43-2.36 (m, 1H), 2.34-2.21 (m, 3H), 2.18-2.07 (m, 3H), 1.86-1.79 (m, 1H), 1.69-1.58 (m, 4H), 1.51 (d, J=6.5 Hz, 3H), 1.45-1.30 (m, 12H), 1.05 (s, 9H). HRMS m/z [M+H]⁺ calcd for C₅₄H₇₂N₉O₆S⁺ 974.5321, found 974.5359.

### Example 81

### Synthesis of LQ076-123

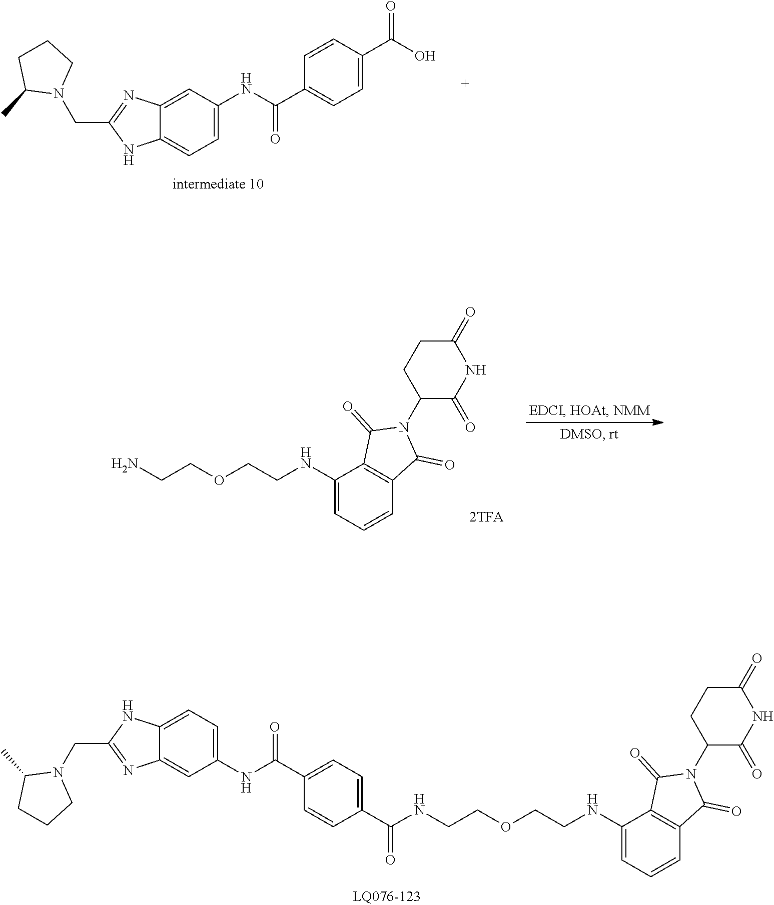 [SMILES: C[C@H]1CCCN1Cc1nc2cc(NC(=O)c3ccc(C(=O)O)cc3)ccc2[nH]1 ; NCCOCCNc1cccc2c1C(=O)N(C1CCC(=O)NC1=O)C2=O ; C[C@H]1CCCN1Cc1nc2cc(NC(=O)c3ccc(C(=O)NCCOCCNc4cccc5c4C(=O)N(C4CCC(=O)NC4=O)C5=O)cc3)ccc2[nH]1]

LQ076-123 was synthesized following the standard procedure for preparing LQ076-105 from intermediate 10 (10 mg, 0.02 mmol), 4-((2-(2-aminoethoxy)ethyl)amino)-2-(2,6-dioxopiperidin-3-yl)isoindoline-1,3-dione (10.1 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-123 was obtained as yellow solid in TFA salt form (11 mg, 58%). ¹H NMR (600 MHz, Methanol-d₄) δ 8.31 (d, J=2.0 Hz, 1H), 8.00 (d, J=8.1 Hz, 2H), 7.90 (d, J=8.2 Hz, 2H), 7.68 (d, J=8.7 Hz, 1H), 7.58 (dd, J=8.6, 1.9 Hz, 1H), 7.49 (dd, J=8.6, 7.1 Hz, 1H), 7.09 (d, J=8.6 Hz, 1H), 6.97 (d, J=7.0 Hz, 1H), 5.00 (dd, J=12.9, 5.5 Hz, 1H), 4.82 (d, J=14.6 Hz, 1H), 4.58 (d, J=14.6 Hz, 1H), 3.79-3.71 (m, 6H), 3.63 (t, J=5.3 Hz, 2H), 3.55-3.51 (m, 2H), 3.50-3.44 (m, 1H), 2.89-2.82 (m, 1H), 2.74-2.63 (m, 2H), 2.43-2.36 (m, 1H), 2.17-2.05 (m, 3H), 1.86-1.79 (m, 1H), 1.51 (d, J=6.5 Hz, 3H). HRMS m/z [M+H]⁺ calcd for C₃₈H₄₁N₈O₇⁺ 721.3461, found 721.3495.

### Example 82

### Synthesis of LQ076-124

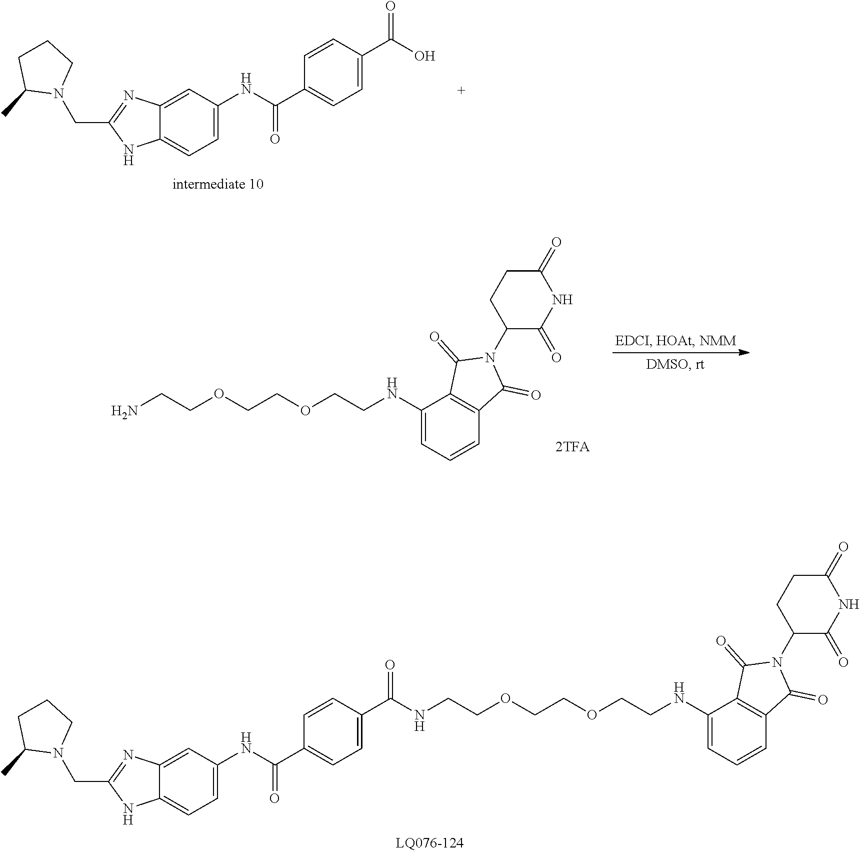 [SMILES: C[C@H]1CCCN1Cc1nc2cc(NC(=O)c3ccc(C(=O)O)cc3)ccc2[nH]1 ; NCCOCCOCCNc1cccc2c1C(=O)N(C1CCC(=O)NC1=O)C2=O ; C[C@H]1CCCN1Cc1nc2cc(NC(=O)c3ccc(C(=O)NCCOCCOCCNc4cccc5c4C(=O)N(C4CCC(=O)NC4=O)C5=O)cc3)ccc2[nH]1]

LQ076-124 was synthesized following the standard procedure for preparing LQ076-105 from intermediate 10 (10 mg, 0.02 mmol), 4-((2-(2-(2-aminoethoxy)ethoxy)ethyl)amino)-2-(2,6-dioxopiperidin-3-yl)isoindoline-1,3-dione (10.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-124 was obtained as yellow solid in TFA salt form (12.4 mg, 63%). ¹H NMR (600 MHz, Methanol-d₄) δ 8.29 (d, J=2.0 Hz, 1H), 7.97 (d, J=8.3 Hz, 2H), 7.93 (d, J=8.3 Hz, 2H), 7.68 (d, J=8.7 Hz, 1H), 7.55 (dd, J=8.7, 1.9 Hz, 1H), 7.51 (dd, J=8.5, 7.1 Hz, 1H), 7.03 (d, J=8.6 Hz, 1H), 6.99 (d, J=7.1 Hz, 1H), 5.03 (dd, J=12.8, 5.5 Hz, 1H), 4.81 (d, J=14.6 Hz, 1H), 4.57 (d, J=14.6 Hz, 1H), 3.79-3.70 (m, 10H), 3.65-3.61 (m, 2H), 3.50-3.43 (m, 3H), 2.86-2.79 (m, 1H), 2.74-2.63 (m, 2H), 2.44-2.37 (m, 1H), 2.18-2.06 (m, 3H), 1.86-1.79 (m, 1H), 1.51 (d, J=6.5 Hz, 3H). HRMS m/z [M+H]⁺ calcd for C₄₀H₄₅N₈O₈⁺ 765.3355, found 765.3350.

### Example 83

### Synthesis of LQ076-125

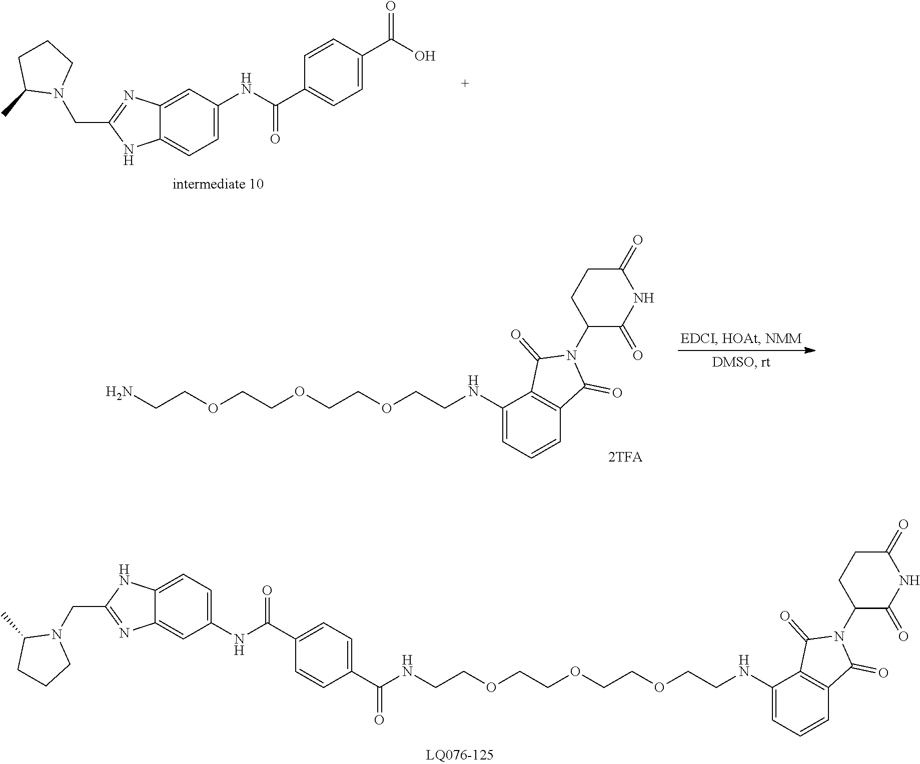 [SMILES: C[C@H]1CCCN1Cc1nc2cc(NC(=O)c3ccc(C(=O)O)cc3)ccc2[nH]1 ; NCCOCCOCCOCCNc1cccc2c1C(=O)N(C1CCC(=O)NC1=O)C2=O ; C[C@H]1CCCN1Cc1nc2cc(NC(=O)c3ccc(C(=O)NCCOCCOCCOCCNc4cccc5c4C(=O)N(C4CCC(=O)NC4=O)C5=O)cc3)ccc2[nH]1]

LQ076-125 was synthesized following the standard procedure for preparing LQ076-105 from intermediate 10 (10 mg, 0.02 mmol), 4-((2-(2-(2-(2-aminoethoxy)ethoxy)ethoxy)ethyl)amino)-2-(2,6-dioxopiperidin-3-yl)isoindoline-1,3-dione (11.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-125 was obtained as yellow solid in TFA salt form (13.4 mg, 65%). ¹H NMR (600 MHz, Methanol-d₄) δ 8.29 (d, J=2.0 Hz, 1H), 8.04-8.00 (m, 2H), 7.98-7.94 (m, 2H), 7.68 (d, J=8.8 Hz, 1H), 7.55 (dd, J=8.7, 2.0 Hz, 1H), 7.49 (dd, J=8.6, 7.0 Hz, 1H), 7.03 (d, J=8.6 Hz, 1H), 6.98 (d, J=7.0 Hz, 1H), 5.03 (dd, J=12.8, 5.5 Hz, 1H), 4.83 (d, J=14.7 Hz, 1H), 4.59 (d, J=14.6 Hz, 1H), 3.78-3.71 (m, 2H), 3.70-3.59 (m, 14H), 3.50-3.42 (m, 3H), 2.87-2.79 (m, 1H), 2.75-2.64 (m, 2H), 2.43-2.37 (m, 1H), 2.18-2.07 (m, 3H), 1.86-1.79 (m, 1H), 1.51 (d, J=6.5 Hz, 3H). HRMS m/z [M+H]⁺ calcd for C₄₂H₄₉N₈O₉⁺ 809.3617, found 809.3636.

### Example 84

### Synthesis of LQ076-126

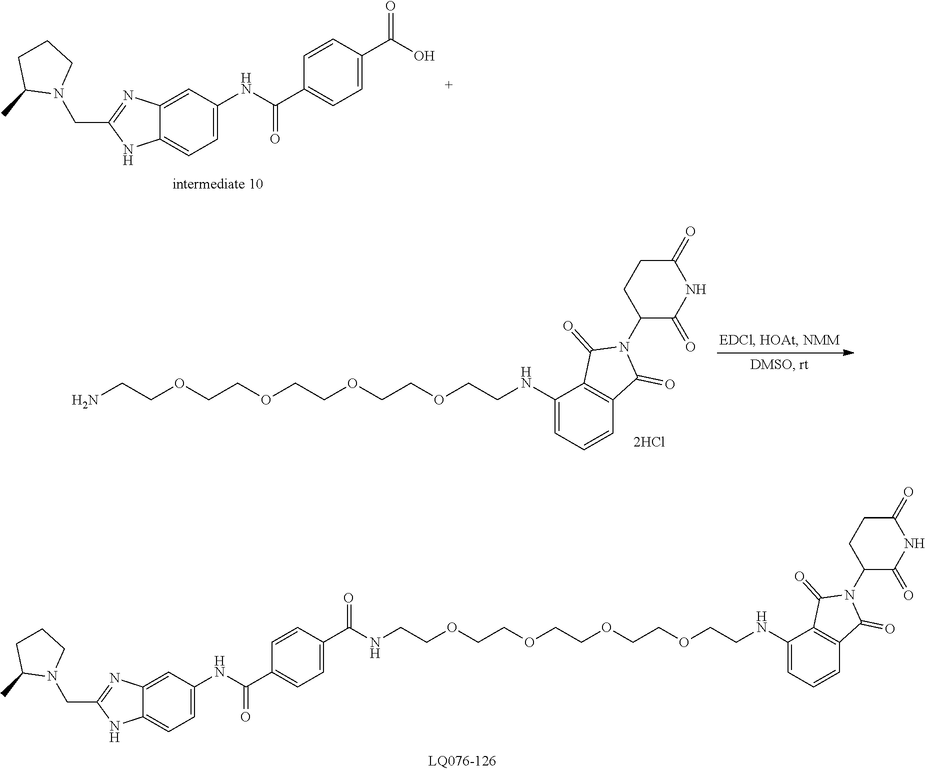 [SMILES: C[C@H]1CCCN1Cc1nc2cc(NC(=O)c3ccc(C(=O)O)cc3)ccc2[nH]1 ; NCCOCCOCCOCCOCCNc1cccc2c1C(=O)N(C1CCC(=O)NC1=O)C2=O ; C[C@H]1CCCN1Cc1nc2cc(NC(=O)c3ccc(C(=O)NCCOCCOCCOCCOCCNc4cccc5c4C(=O)N(C4CCC(=O)NC4=O)C5=O)cc3)ccc2[nH]1]

LQ076-126 was synthesized following the standard procedure for preparing LQ076-105 from intermediate 10 (10 mg, 0.02 mmol), 4-((14-amino-3,6,9,12-tetraoxatetradecyl)amino)-2-(2,6-dioxopiperidin-3-yl)isoindoline-1,3-dione (13.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-126 was obtained as yellow solid in TFA salt form (15.0 mg, 70%). ¹H NMR (600 MHz, Methanol-d₄) δ 8.28 (d, J=1.9 Hz, 1H), 8.04 (d, J=8.1 Hz, 2H), 7.98 (d, J=8.3 Hz, 2H), 7.66 (d, J=8.8 Hz, 1H), 7.54 (dd, J=8.8, 2.0 Hz, 1H), 7.49 (dd, J=8.6, 7.1 Hz, 1H), 7.03 (d, J=8.6 Hz, 1H), 6.99 (d, J=7.1 Hz, 1H), 5.04 (dd, J=12.7, 5.5 Hz, 1H), 4.81 (d, J=14.6 Hz, 1H), 4.57 (d, J=14.6 Hz, 1H), 3.78-3.59 (m, 20H), 3.48-3.44 (m, 3H), 2.88-2.80 (m, 1H), 2.76-2.65 (m, 2H), 2.43-2.36 (m, 1H), 2.18-2.07 (m, 3H), 1.86-1.78 (m, 1H), 1.51 (d, J=6.5 Hz, 3H). HRMS m/z [M+H]⁺ calcd for C₄₄H₅₃N₈O₁₀⁺ 853.3879, found 853.3920.

### Example 85

### Synthesis of LQ076-127

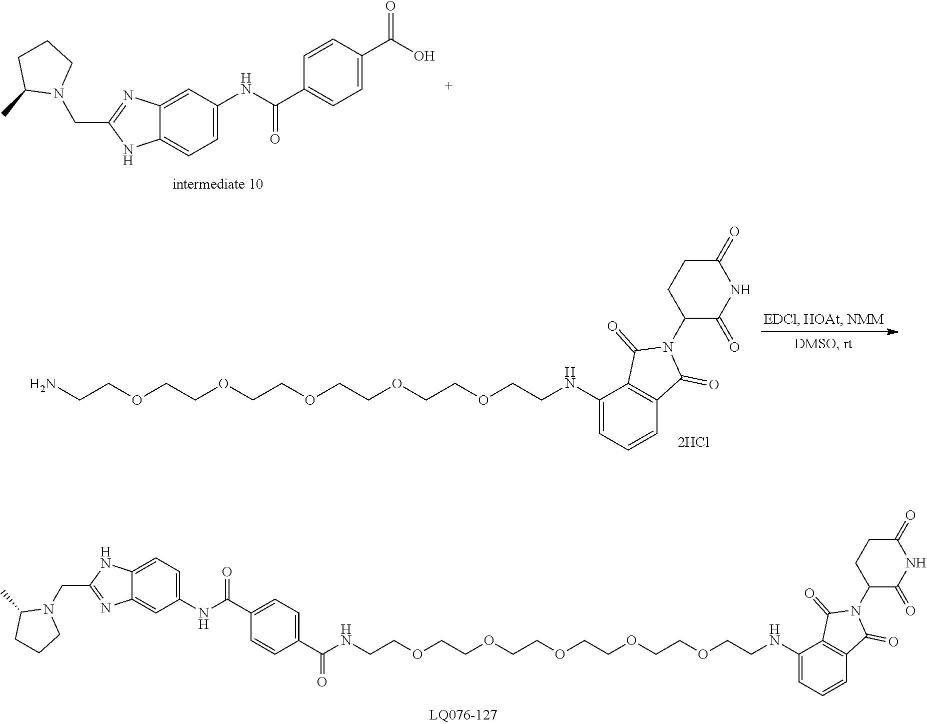 [SMILES: C[C@H]1CCCN1Cc1nc2cc(NC(=O)c3ccc(C(=O)O)cc3)ccc2[nH]1 ; NCCOCCOCCOCCOCCOCCNc1cccc2c1C(=O)N(C1CCC(=O)NC1=O)C2=O ; C[C@H]1CCCN1Cc1nc2cc(NC(=O)c3ccc(C(=O)NCCOCCOCCOCCOCCOCCNc4cccc5c4C(=O)N(C4CCC(=O)NC4=O)C5=O)cc3)ccc2[nH]1]

LQ076-127 was synthesized following the standard procedure for preparing LQ076-105 from intermediate 10 (10 mg, 0.02 mmol), 4-((17-amino-3,6,9,12,15-pentaoxaheptadecyl)amino)-2-(2,6-dioxopiperidin-3-yl)isoindoline-1,3-dione (13.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-127 was obtained as yellow solid in TFA salt form (15.8 mg, 70%). ¹H NMR (600 MHz, Methanol-d₄) δ 8.30 (d, J=2.0 Hz, 1H), 8.06-8.03 (m, 2H), 8.00-7.96 (m, 2H), 7.67 (d, J=8.8 Hz, 1H), 7.57 (dd, J=8.8, 2.0 Hz, 1H), 7.50 (dd, J=8.6, 7.1 Hz, 1H), 7.03 (d, J=8.6 Hz, 1H), 7.00 (d, J=7.1 Hz, 1H), 5.04 (dd, J=12.8, 5.5 Hz, 1H), 4.83 (d, J=14.6 Hz, 1H), 4.59 (d, J=14.6 Hz, 1H), 3.78-3.58 (m, 24H), 3.48-3.43 (m, 3H), 2.88-2.81 (m, 1H), 2.76-2.66 (m, 2H), 2.43-2.37 (m, 1H), 2.18-2.07 (m, 3H), 1.86-1.79 (m, 1H), 1.51 (d, J=6.5 Hz, 3H). HRMS m/z [M+H]⁺ calcd for C₄₆H₅₇N₈O₁₁⁺ 897.4141, found 897.4174.

### Example 86

### Synthesis of LQ076-128

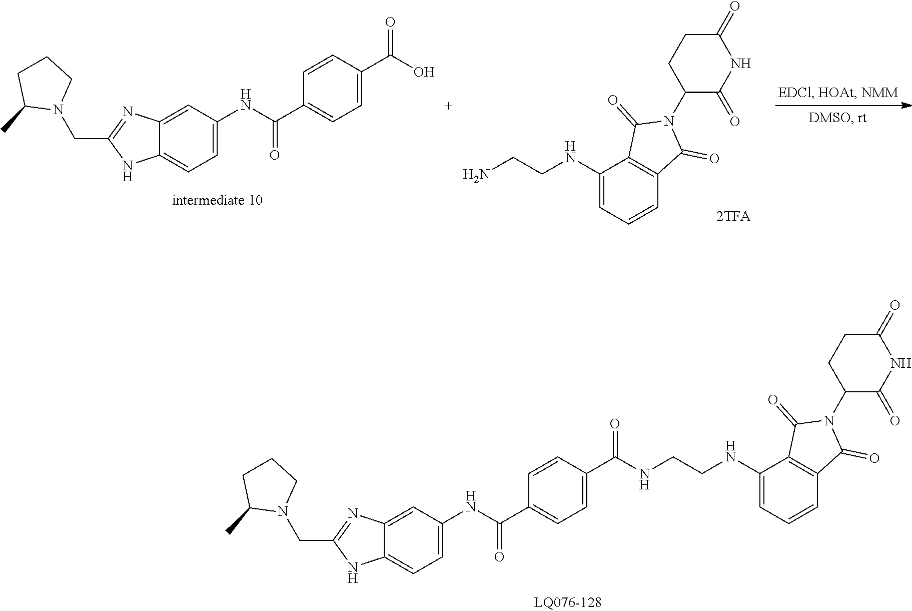 [SMILES: C[C@H]1CCCN1Cc1nc2cc(NC(=O)c3ccc(C(=O)O)cc3)ccc2[nH]1 ; NCCNc1cccc2c1C(=O)N(C1CCC(=O)NC1=O)C2=O ; C[C@H]1CCCN1Cc1nc2cc(NC(=O)c3ccc(C(=O)NCCNc4cccc5c4C(=O)N(C4CCC(=O)NC4=O)C5=O)cc3)ccc2[nH]1]

LQ076-128 was synthesized following the standard procedure for preparing LQ076-105 from intermediate 10 (10 mg, 0.02 mmol), 4-((2-aminoethyl)amino)-2-(2,6-dioxopiperidin-3-yl)isoindoline-1,3-dione (9.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-128 was obtained as yellow solid in TFA salt form (11.7 mg, 65%). ¹H NMR (600 MHz, Methanol-d₄) δ 8.30 (d, J=2.1 Hz, 1H), 8.02 (d, J=8.2 Hz, 2H), 7.92 (d, J=8.3 Hz, 2H), 7.85-7.81 (m, 1H), 7.68 (d, J=8.6 Hz, 1H), 7.58-7.48 (m, 2H), 7.20 (d, J=8.6 Hz, 1H), 7.04 (d, J=7.0 Hz, 1H), 5.06 (dd, J=12.8, 5.5 Hz, 1H), 4.83 (d, J=14.6 Hz, 1H), 4.59 (d, J=14.6 Hz, 1H), 3.78-3.70 (m, 3H), 3.68-3.61 (m, 3H), 3.49-3.43 (m, 1H), 2.89-2.82 (m, 1H), 2.77-2.67 (m, 2H), 2.43-2.36 (m, 1H), 2.18-2.06 (m, 3H), 1.86-1.79 (m, 1H), 1.51 (d, J=6.5 Hz, 3H). HRMS m/z [M+H]⁺ calcd for C₃₆H₃₇N₈O₆⁺ 677.2831, found 677.2857.

### Example 87

### Synthesis of LQ076-129

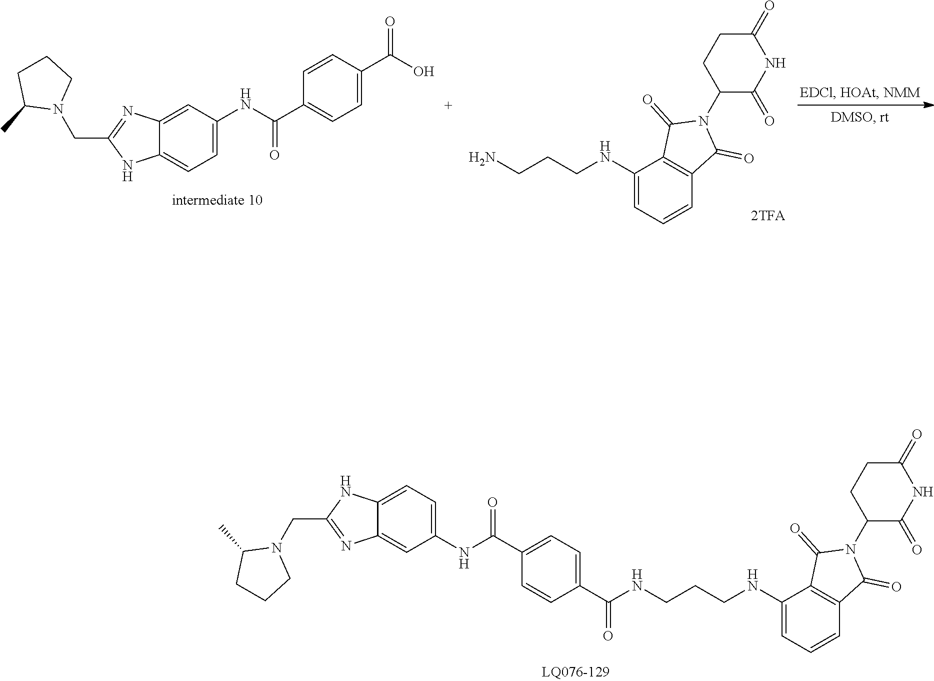 [SMILES: C[C@H]1CCCN1Cc1nc2cc(NC(=O)c3ccc(C(=O)O)cc3)ccc2[nH]1 ; NCCCNc1cccc2c1C(=O)N(C1CCC(=O)NC1=O)C2=O ; C[C@H]1CCCN1Cc1nc2cc(NC(=O)c3ccc(C(=O)NCCCNc4cccc5c4C(=O)N(C4CCC(=O)NC4=O)C5=O)cc3)ccc2[nH]1]

LQ076-129 was synthesized following the standard procedure for preparing LQ076-105 from intermediate 10 (10 mg, 0.02 mmol), 4-((3-aminopropyl)amino)-2-(2,6-dioxopiperidin-3-yl)isoindoline-1,3-dione (9.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-129 was obtained as yellow solid in TFA salt form (12.3 mg, 67%). ¹H NMR (600 MHz, Methanol-d₄) δ 8.30 (d, J=2.0 Hz, 1H), 8.04 (d, J=8.1 Hz, 2H), 7.96 (d, J=8.2 Hz, 2H), 7.68 (d, J=8.8 Hz, 1H), 7.58-7.53 (m, 2H), 7.09 (d, J=8.6 Hz, 1H), 7.05 (d, J=7.1 Hz, 1H), 5.05 (dd, J=12.7, 5.5 Hz, 1H), 4.81 (d, J=14.6 Hz, 1H), 4.57 (d, J=14.6 Hz, 1H), 3.78-3.71 (m, 2H), 3.57 (t, J=6.7 Hz, 2H), 3.50-3.45 (m, 3H), 2.90-2.83 (m, 1H), 2.77-2.66 (m, 2H), 2.43-2.37 (m, 1H), 2.18-2.06 (m, 3H), 2.03-1.97 (m, 2H), 1.86-1.79 (m, 1H), 1.51 (d, J=6.6 Hz, 3H). HRMS m/z [M+H]⁺ calcd for C₃₇H₃₉N₈O₆⁺ 691.2987, found 691.3031.

### Example 88

### Synthesis of LQ076-130

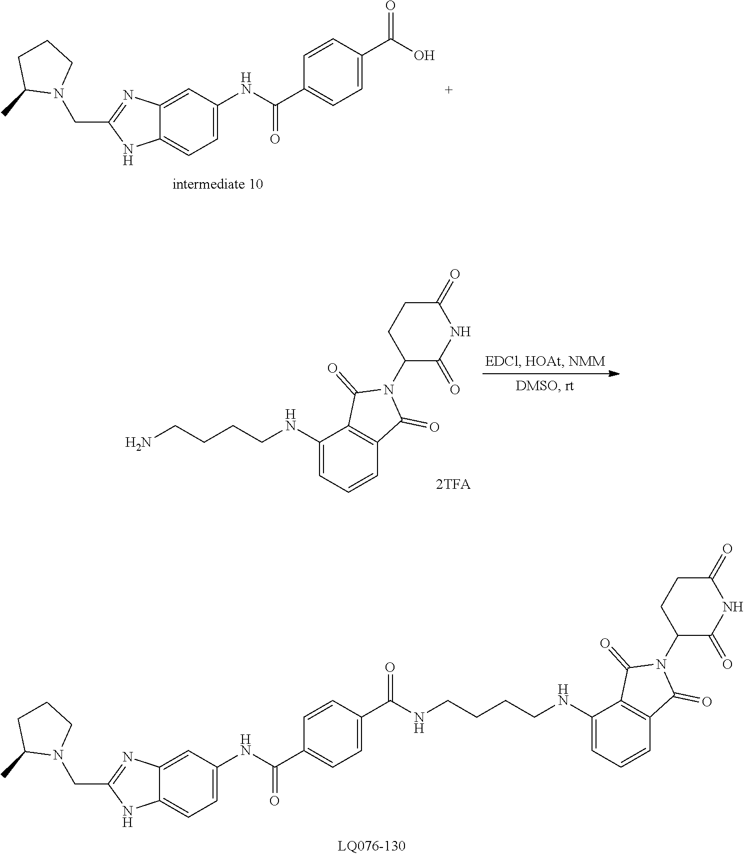 [SMILES: C[C@H]1CCCN1Cc1nc2cc(NC(=O)c3ccc(C(=O)O)cc3)ccc2[nH]1 ; NCCCCNc1cccc2c1C(=O)N(C1CCC(=O)NC1=O)C2=O ; C[C@H]1CCCN1Cc1nc2cc(NC(=O)c3ccc(C(=O)NCCCCNc4cccc5c4C(=O)N(C4CCC(=O)NC4=O)C5=O)cc3)ccc2[nH]1]

LQ076-130 was synthesized following the standard procedure for preparing LQ076-105 from intermediate 10 (10 mg, 0.02 mmol), 4-((4-aminobutyl)amino)-2-(2,6-dioxopiperidin-3-yl)isoindoline-1,3-dione (10.1 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-130 was obtained as yellow solid in TFA salt form (14.9 mg, 80%). ¹H NMR (600 MHz, Methanol-d₄) δ 8.30 (d, J=2.0 Hz, 1H), 8.04 (d, J=8.3 Hz, 2H), 7.95 (d, J=8.2 Hz, 2H), 7.68 (d, J=8.7 Hz, 1H), 7.57-7.53 (m, 2H), 7.08 (d, J=8.6 Hz, 1H), 7.04 (d, J=7.1 Hz, 1H), 5.07 (dd, J=12.8, 5.5 Hz, 1H), 4.80 (d, J=14.6 Hz, 1H), 4.56 (d, J=14.6 Hz, 1H), 3.77-3.71 (m, 2H), 3.51-3.41 (m, 5H), 2.90-2.83 (m, 1H), 2.78-2.68 (m, 2H), 2.43-2.37 (m, 1H), 2.18-2.08 (m, 3H), 1.85-1.76 (m, 5H), 1.51 (d, J=6.5 Hz, 3H). HRMS m/z [M+H]⁺ calcd for C₃₈H₄₁N₈O₆⁺ 705.3144, found 705.3162.

### Example 89

### Synthesis of LQ076-131

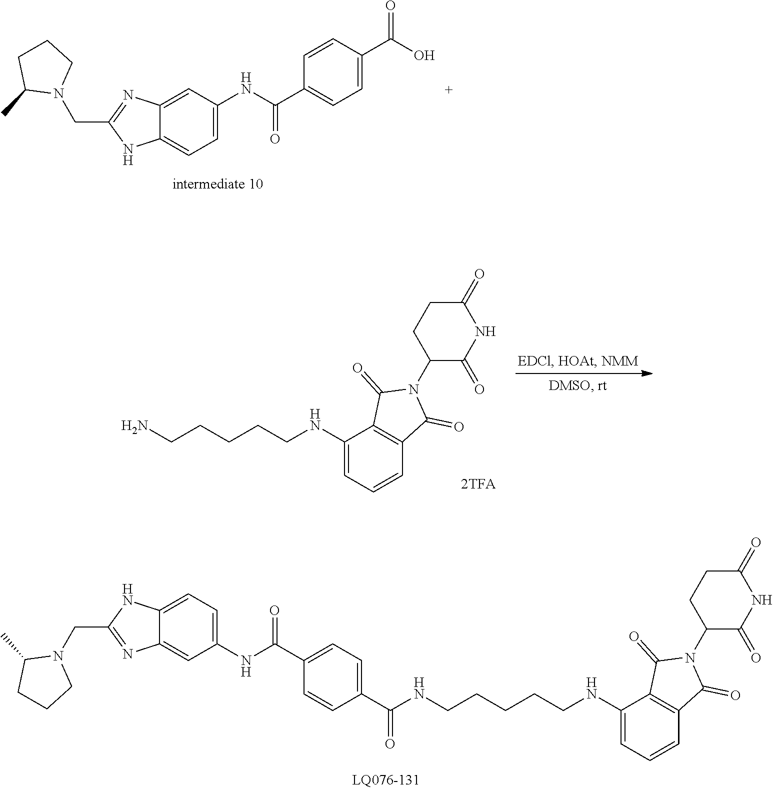 [SMILES: C[C@H]1CCCN1Cc1nc2cc(NC(=O)c3ccc(C(=O)O)cc3)ccc2[nH]1 ; NCCCCCNc1cccc2c1C(=O)N(C1CCC(=O)NC1=O)C2=O ; C[C@H]1CCCN1Cc1nc2cc(NC(=O)c3ccc(C(=O)NCCCCCNc4cccc5c4C(=O)N(C4CCC(=O)NC4=O)C5=O)cc3)ccc2[nH]1]

LQ076-131 was synthesized following the standard procedure for preparing LQ076-105 from intermediate 10 (10 mg, 0.02 mmol), 4-((5-aminopentyl)amino)-2-(2,6-dioxopiperidin-3-yl)isoindoline-1,3-dione (10.6 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-131 was obtained as yellow solid in TFA salt form (14.3 mg, 75%). ¹H NMR (600 MHz, Methanol-d₄) δ 8.31 (d, J=2.0 Hz, 1H), 8.03 (d, J=8.3 Hz, 2H), 7.95-7.91 (m, 2H), 7.68 (d, J=8.7 Hz, 1H), 7.58 (dd, J=8.8, 2.0 Hz, 1H), 7.53 (dd, J=8.6, 7.1 Hz, 1H), 7.04 (d, J=8.6 Hz, 1H), 7.01 (d, J=7.0 Hz, 1H), 5.05 (dd, J=12.8, 5.5 Hz, 1H), 4.82 (d, J=14.6 Hz, 1H), 4.58 (d, J=14.6 Hz, 1H), 3.78-3.71 (m, 2H), 3.48-3.43 (m, 3H), 3.36 (t, J=6.9 Hz, 2H), 2.90-2.82 (m, 1H), 2.76-2.66 (m, 2H), 2.43-2.36 (m, 1H), 2.18-2.06 (m, 3H), 1.86-1.79 (m, 1H), 1.77-1.70 (m, 4H), 1.58-1.53 (m, 2H), 1.51 (d, J=6.6 Hz, 3H). HRMS m/z [M+H]⁺ calcd for C₃₉H₄₃N₈O₆⁺ 719.3300, found 719.3340.

### Example 90

### Synthesis of LQ076-132

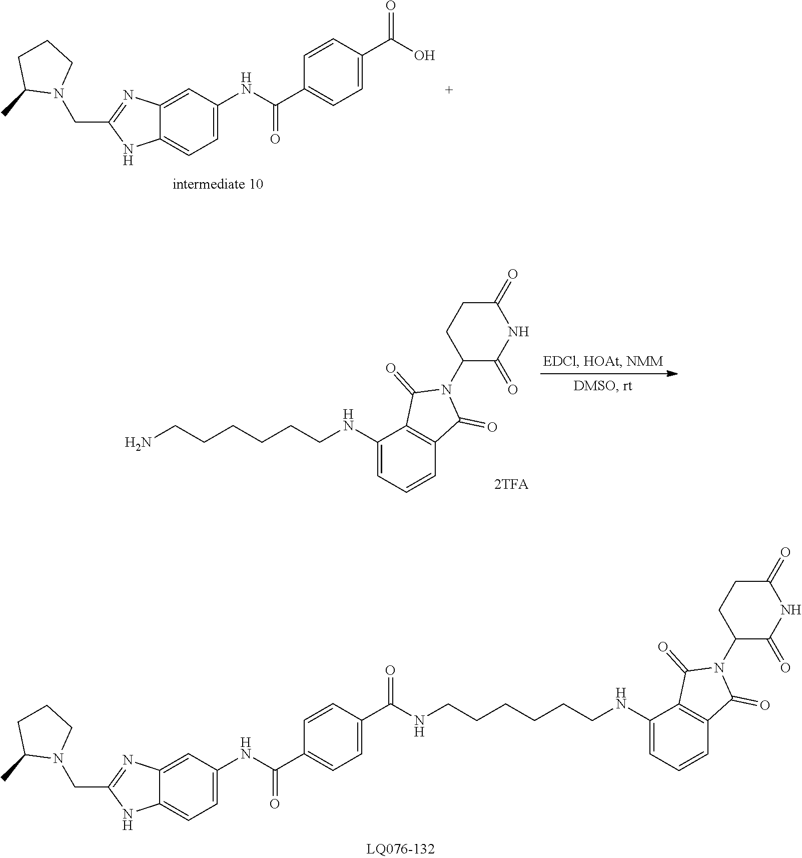 [SMILES: C[C@H]1CCCN1Cc1nc2cc(NC(=O)c3ccc(C(=O)O)cc3)ccc2[nH]1 ; NCCCCCCNc1cccc2c1C(=O)N(C1CCC(=O)NC1=O)C2=O ; C[C@H]1CCCN1Cc1nc2cc(NC(=O)c3ccc(C(=O)NCCCCCCNc4cccc5c4C(=O)N(C4CCC(=O)NC4=O)C5=O)cc3)ccc2[nH]1]

LQ076-132 was synthesized following the standard procedure for preparing LQ076-105 from intermediate 10 (10 mg, 0.02 mmol), 4-((6-aminohexyl)amino)-2-(2,6-dioxopiperidin-3-yl)isoindoline-1,3-dione (10 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-132 was obtained as yellow solid in TFA salt form (13.7 mg, 71%). ¹H NMR (600 MHz, Methanol-d₄) δ 8.31 (d, J=1.9 Hz, 1H), 8.04 (d, J=8.2 Hz, 2H), 7.96 (d, J=8.4 Hz, 2H), 7.69 (d, J=8.7 Hz, 1H), 7.59-7.53 (m, 2H), 7.06-7.02 (m, 2H), 5.06 (dd, J=12.5, 5.5 Hz, 1H), 4.82 (d, J=14.6 Hz, 1H), 4.58 (d, J=14.6 Hz, 1H), 3.78-3.71 (m, 2H), 3.50-3.41 (m, 3H), 3.37-3.34 (m, 2H), 2.90-2.83 (m, 1H), 2.78-2.67 (m, 2H), 2.43-2.36 (m, 1H), 2.18-2.07 (m, 3H), 1.86-1.79 (m, 1H), 1.75-1.67 (m, 4H), 1.55-1.47 (m, 7H). HRMS m/z [M+H]⁺ calcd for C₄₀H₄₅N₈O₆⁺ 733.3457, found 733.3479.

### Example 91

### Synthesis of LQ076-133

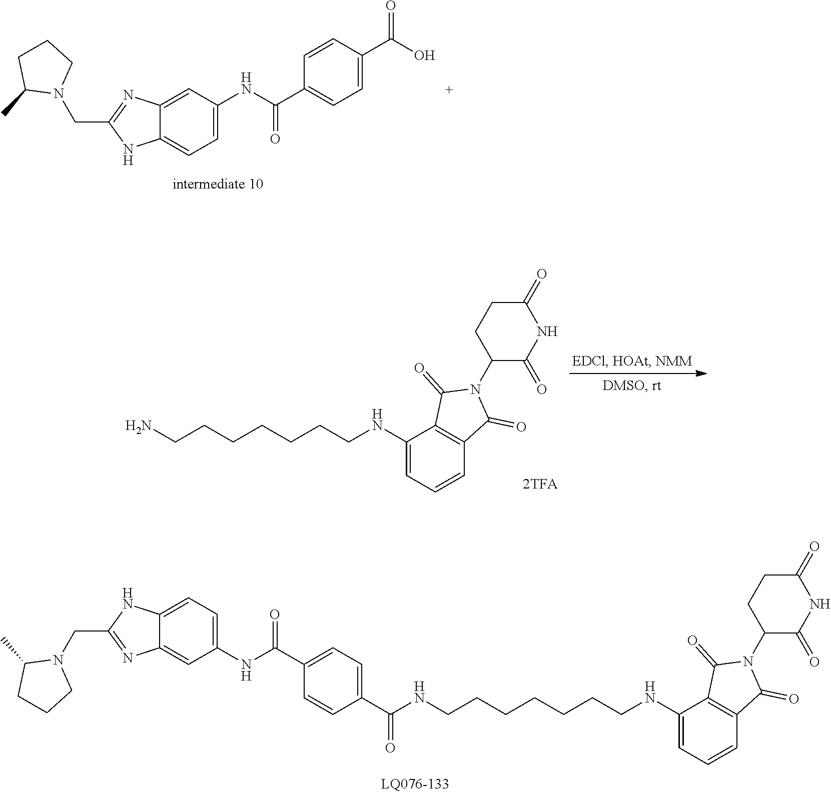 [SMILES: C[C@H]1CCCN1Cc1nc2cc(NC(=O)c3ccc(C(=O)O)cc3)ccc2[nH]1 ; NCCCCCCCNc1cccc2c1C(=O)N(C1CCC(=O)NC1=O)C2=O ; C[C@H]1CCCN1Cc1nc2cc(NC(=O)c3ccc(C(=O)NCCCCCCCNc4cccc5c4C(=O)N(C4CCC(=O)NC4=O)C5=O)cc3)ccc2[nH]1]

LQ076-133 was synthesized following the standard procedure for preparing LQ076-105 from intermediate 10 (10 mg, 0.02 mmol), 4-((7-aminoheptyl)amino)-2-(2,6-dioxopiperidin-3-yl)isoindoline-1,3-dione (10.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-133 was obtained as yellow solid in TFA salt form (14.6 mg, 75%). ¹H NMR (600 MHz, Methanol-d₄) δ 8.31 (d, J=2.0 Hz, 1H), 8.04 (d, J=8.2 Hz, 2H), 7.97-7.94 (m, 2H), 7.69 (d, J=8.7 Hz, 1H), 7.58 (dd, J=8.7, 2.0 Hz, 1H), 7.54 (dd, J=8.5, 7.0 Hz, 1H), 7.05-7.00 (m, 2H), 5.05 (dd, J=12.7, 5.5 Hz, 1H), 4.83 (d, J=14.6 Hz, 1H), 4.59 (d, J=14.6 Hz, 1H), 3.78-3.71 (m, 2H), 3.50-3.40 (m, 3H), 3.34-3.33 (m, 2H), 2.88-2.80 (m, 1H), 2.76-2.67 (m, 2H), 2.43-2.37 (m, 1H), 2.18-2.07 (m, 3H), 1.86-1.79 (m, 1H), 1.71-1.64 (m, 4H), 1.53-1.42 (m, 9H). HRMS m/z [M+H]⁺ calcd for C₄₁H₄₇N₈O₆⁺ 747.3613, found 747.3639.

### Example 92

### Synthesis of LQ076-134

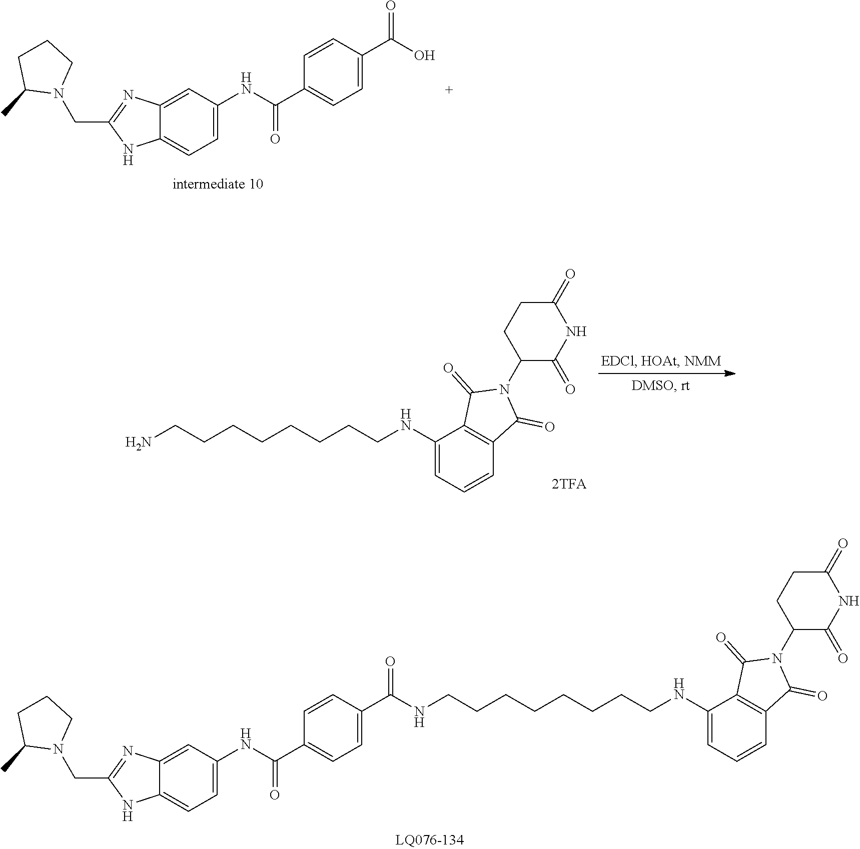 [SMILES: C[C@H]1CCCN1Cc1nc2cc(NC(=O)c3ccc(C(=O)O)cc3)ccc2[nH]1 ; NCCCCCCCCNc1cccc2c1C(=O)N(C1CCC(=O)NC1=O)C2=O ; C[C@H]1CCCN1Cc1nc2cc(NC(=O)c3ccc(C(=O)NCCCCCCCCNc4cccc5c4C(=O)N(C4CCC(=O)NC4=O)C5=O)cc3)ccc2[nH]1]

LQ076-134 was synthesized following the standard procedure for preparing LQ076-105 from intermediate 10 (10 mg, 0.02 mmol), 4-((8-aminooctyl)amino)-2-(2,6-dioxopiperidin-3-yl)isoindoline-1,3-dione (11.4 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-134 was obtained as yellow solid in TFA salt form (15.1 mg, 76%). ¹H NMR (600 MHz, Methanol-d₄) δ 8.30 (d, J=2.0 Hz, 1H), 8.05 (d, J=8.1 Hz, 2H), 7.96 (d, J=8.3 Hz, 2H), 7.68 (d, J=8.8 Hz, 1H), 7.58-7.52 (m, 2H), 7.05-7.01 (m, 2H), 5.06 (dd, J=12.4, 5.5 Hz, 1H), 4.81 (d, J=14.6 Hz, 1H), 4.57 (d, J=14.6 Hz, 1H), 3.78-3.71 (m, 2H), 3.50-3.40 (m, 3H), 3.32-3.29 (m, 2H), 2.90-2.82 (m, 1H), 2.78-2.68 (m, 2H), 2.43-2.36 (m, 1H), 2.18-2.07 20 (m, 3H), 1.86-1.78 (m, 1H), 1.67 (q, J=7.5 Hz, 4H), 1.51 (d, J=6.5 Hz, 3H), 1.49-1.39 (m, 8H). HRMS m/z [M+H]⁺ calcd for C₄₂H₄₉N₈O₆⁺ 761.3770, found 761.3802.

### Example 93

### Synthesis of Intermediate 14

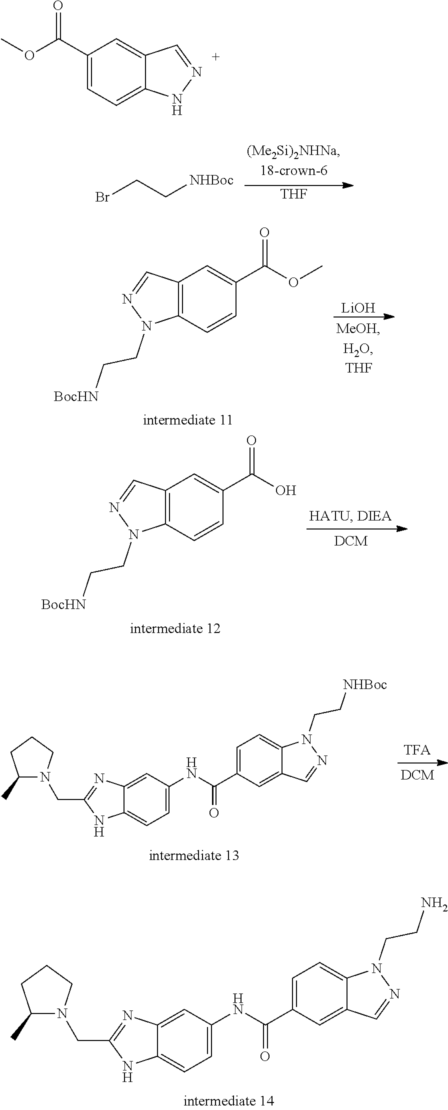

### Intermediate 11: Methyl 1-(2-((tert-butoxycarbonyl)amino)ethyl)-1H-indazole-5-carboxylate

Methyl 1H-indazole-5-carboxylate (0.87 g, 4.9 mmol) and 18-crown-6 (20 mg) were added to 20 5 mL dry THF. Sodium bis(trimethylsilyl)amide (7.3 mL, 7.3 mmol, 1.0 M in THF) was added via syringe, followed by tert-Butyl (2-bromoethyl)carbamate (1.4 g, 6.4 mmol). The reaction was heated at reflux for 24 hr, cooled, and concentrated under vacuum. The residue was partitioned between ethyl acetate and water, separated, and the aqueous layer extracted with ethyl acetate. The combined organics were washed with brine, dried over sodium sulfate and concentrated. The resulting residue was purified by silica gel flash chromatography to give the two separate products. The one is intermediate 11 (750 mg, 48%). ¹H NMR (600 MHz, Methanol-d₄) δ 8.53 (s, 1H), 8.20 (s, 1H), 8.04 (d, J=9.0 Hz, 1H), 7.62 (d, J=8.9 Hz, 1H), 4.54 (t, J=5.9 Hz, 2H), 3.95 (s, 3H), 3.52 (t, J=5.9 Hz, 2H), 1.32 (s, 9H). MS (ESI): m/z 320.1 [M+H]⁺. The others is 2-substitute products. ¹H NMR (600 MHz, Methanol-d₄) δ 8.54 (s, 1H), 8.41 (s, 1H), 7.89 (d, J=9.1 Hz, 1H), 7.66 (d, J=9.1 Hz, 1H), 4.56 (t, J=5.9 Hz, 2H), 3.94 (s, 3H), 3.62 (t, J=5.9 Hz, 2H), 1.38 (s, 9H).

### Intermediate 12: 1-(2-((tert-butoxycarbonyl)amino)ethyl)-1H-indazole-5-carboxylic acid

Intermediate 12 was synthesized according to the procedures for the preparation of intermediate 4 as a white solid in 85% yield. MS (ESI): m/z 306.0 [M+H]⁺.

### Intermediate 13: tert-butyl (S)-(2-(5-((2-((2-methylpyrrolidin-1-yl)methyl)-1H-benzo[d]imidazol-5-yl)carbamoyl)-1H-indazol-1-yl)ethyl)carbamate

Intermediate 13 was synthesized according to the procedures for the preparation of intermediate 9 as a white solid in 69% yield. MS (ESI): m/z 518.3 [M+H]⁺.

### Intermediate 14: (S)-1-(2-aminoethyl)-N-(2-((2-methylpyrrolidin-1-yl)methyl)-1H-benzo[d]imidazol-5-yl)-1H-indazole-5-carboxamide

Intermediate 13 (700 mg, 1.35 mmol) was dissolved in 5 mL DCM, to the resulting solution was added 3 mL TFA. After being stirred for 1 h at room temperature, the reaction mixture was concentrated and the residue was purified by reverse phase C18 column (10%-100% methanol/0.1% TFA in water) to afford intermediate 14 as white solid in TFA salt form (600 mg, 86%). ¹H NMR (600 MHz, Methanol-d₄) δ 8.52 (d, J=1.6 Hz, 1H), 8.32-8.29 (m, 2H), 8.11 (dd, J=8.8, 1.7 Hz, 1H), 7.76 (d, J=8.9 Hz, 1H), 7.70 (d, J=8.7 Hz, 1H), 7.61 (dd, J=8.7, 2.0 Hz, 1H), 4.84 (d, J=14.5 Hz, 1H), 4.77 (t, J=5.8 Hz, 2H), 4.61 (d, J=14.6 Hz, 1H), 3.80-3.70 (m, 2H), 3.59 (t, J=5.8 Hz, 2H), 3.51-3.43 (m, 1H), 2.44-2.36 (m, 1H), 2.21-2.05 (m, 2H), 1.88-1.79 (m, 1H), 1.51 (d, J=6.4 Hz, 3H). MS (ESI): m/z 418.4 [M+H]⁺.

### Example 94

### Synthesis of LQ076-135

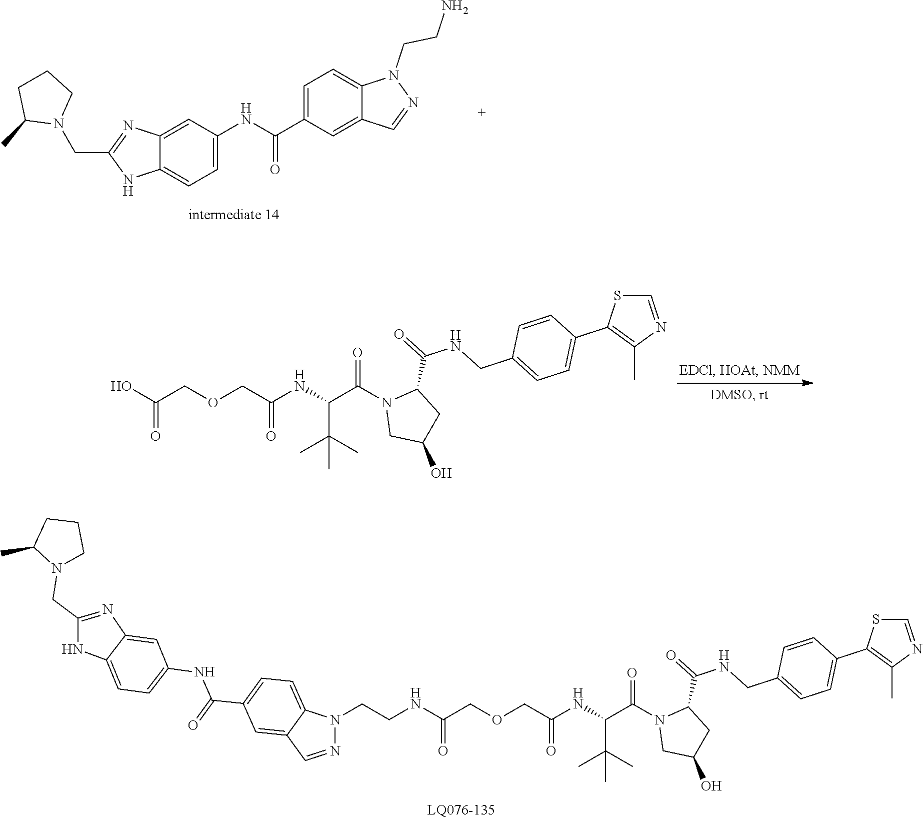 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)COCC(=O)O)C(C)(C)C)cc1 ; Cc1ncsc1-c1ccc(CNC(=O)[C@@H]2C[C@@H](O)CN2C(=O)[C@@H](NC(=O)COCC(=O)NCCn2ncc3cc(C(=O)Nc4ccc5[nH]c(CN6CCC[C@@H]6C)nc5c4)ccc32)C(C)(C)C)cc1]

To a solution of Intermediate 14 (13 mg, 0.02 mmol) in DMSO (1 mL) were added 2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)acetic acid (11.3 mg, 0.02 mmol, 1.0 equiv), EDCI (1-ethyl-3-(3-dimethylaminopropyl)carbodiimide) (5.8 mg, 0.03 mmol, 1.5 equiv), HOAt (1-hydroxy-7-azabenzo-triazole) (4.1 mg, 0.03 mmol, 1.5 equiv), and NMM (N-Methylmorpholine) (6.1 mg, 0.06 mmol, 3.0 equiv). After being stirred overnight at room temperature, the resulting mixture was purified by preparative HPLC (5%-60% acetonitrile/0.1% TFA in H₂O) to afford LQ076-135 as white solid in TFA salt form (19.2 mg, 83%). ¹H NMR (800 MHz, Methanol-d₄) δ 8.95 (s, 1H), 8.44 (s, 1H), 8.31 (s, 1H), 8.22 (s, 1H), 8.03 (d, J=8.9 Hz, 1H), 7.71-7.66 (m, 2H), 7.60 (d, J=8.7 Hz, 1H), 7.47-7.38 (m, 4H), 4.82 (d, J=14.7 Hz, 1H), 4.73-4.70 (m, 1H), 4.68-4.63 (m, 3H), 4.60-4.52 (m, 3H), 4.34 (d, J=15.3 Hz, 1H), 3.96-3.82 (m, 6H), 3.79-3.71 (m, 4H), 3.49-3.44 (m, 1H), 2.46 (s, 3H), 2.42-2.37 (m, 1H), 2.29-2.25 (m, 1H), 2.18-2.08 (m, 4H), 1.86-1.81 (m, 1H), 1.51 (d, J=6.5 Hz, 3H), 1.06 (s, 9H). HRMS m/z [M+H]⁺ calcd for C₄₉H₆₀N₁₁O₇S⁺ 946.4392, found 946.4411.

### Example 95

### Synthesis of LQ076-136

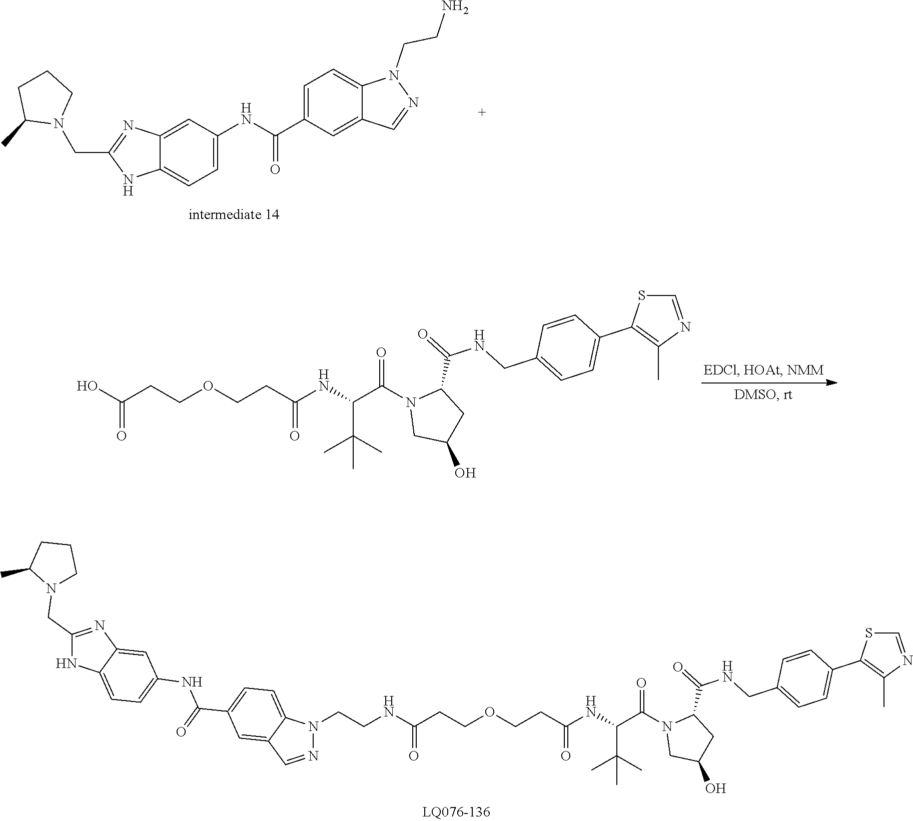 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)CCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCC(=O)NCCn2ncc3cc(C(=O)Nc4ccc5[nH]c(CN6CCC[C@@H]6C)nc5c4)ccc32)C(C)(C)C)cc1]

LQ076-136 was synthesized following the standard procedure for preparing LQ076-135 from intermediate 14 (13 mg, 0.02 mmol), 3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoic acid (11.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-136 was obtained as white solid in TFA salt form (14.9 mg, 62%). ¹H NMR (800 MHz, Methanol-d₄) δ 8.96 (s, 1H), 8.47 (s, 1H), 8.32 (s, 1H), 8.23 (s, 1H), 8.05 (d, J=8.8 Hz, 1H), 7.71-7.66 (m, 2H), 7.59 (d, J=8.8 Hz, 1H), 7.47 (d, J=7.8 Hz, 2H), 7.41 (d, J=7.8 Hz, 2H), 4.81 (d, J=14.8 Hz, 1H), 4.67-4.65 (m, 1H), 4.63-4.49 (m, 6H), 4.37 (d, J=15.3 Hz, 1H), 3.90 (d, J=11.0 Hz, 1H), 3.80 (dd, J=10.9, 4.0 Hz, 1H), 3.77-3.68 (m, 4H), 3.65-3.55 (m, 4H), 3.49-3.44 (m, 1H), 2.48-2.38 (m, 6H), 2.36-2.30 (m, 2H), 2.26-2.22 (m, 1H), 2.18-2.08 (m, 3H), 1.86-1.80 (m, 1H), 1.52 (d, J=6.5 Hz, 3H), 1.04 (s, 9H). HRMS m/z [M+H]+ calcd for C₅₁H₆₄N₁₁O₇S⁺ 974.4705, found 974.4701.

### Example 96

### Synthesis of LQ076-137

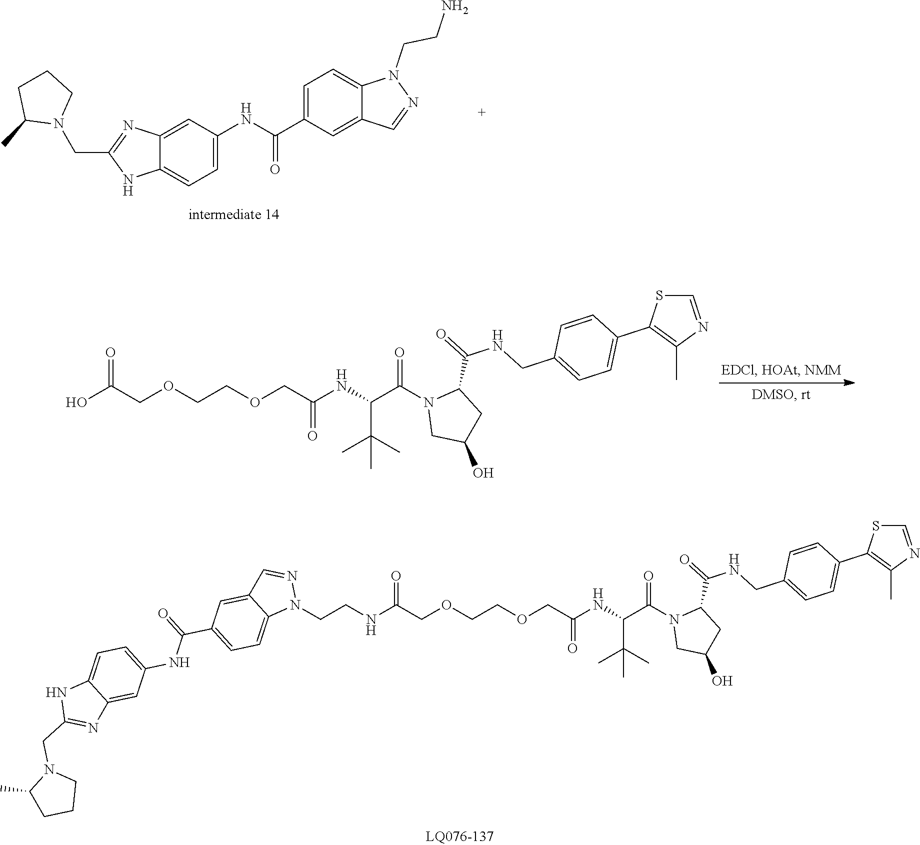 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)COCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCC(=O)NCCn2ncc3cc(C(=O)Nc4ccc5[nH]c(CN6CCC[C@@H]6C)nc5c4)ccc32)C(C)(C)C)cc1]

LQ076-137 was synthesized following the standard procedure for preparing LQ076-135 from intermediate 14 (13 mg, 0.02 mmol), 2-(2-(2-(((S)-1-((2S,4R)-4-hydroxy-2-(3-(4-(4-methylthiazol-5-yl)phenyl)propanoyl)pyrrolidin-1-yl)-3,3-dimethyl-1-oxobutan-2-yl)amino)-2-oxoethoxy)ethoxy)acetic acid (12.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-137 was obtained as white solid in TFA salt form (15.7 mg, 65%). ¹H NMR (800 MHz, Methanol-d₄) δ 9.00 (s, 1H), 8.46 (s, 1H), 8.32 (s, 1H), 8.22 (s, 1H), 8.03 (d, J=8.6 Hz, 1H), 7.71-7.67 (m, 2H), 7.61 (d, J=8.5 Hz, 1H), 7.47-7.36 (m, 4H), 4.83 (d, J=14.7 Hz, 1H), 4.76-4.73 (m, 1H), 4.70-4.58 (m, 4H), 4.55-4.44 (m, 2H), 4.37 (d, J=15.3 Hz, 1H), 4.05-3.99 (m, 1H), 3.95-3.88 (m, 3H), 3.85-3.80 (m, 2H), 3.79-3.70 (m, 4H), 3.65-3.54 (m, 3H), 3.51-3.44 (m, 2H), 2.47 (s, 3H), 2.43-2.38 (m, 1H), 2.31-2.27 (m, 1H), 2.19-2.07 (m, 3H), 1.87-1.81 (m, 1H), 1.52 (d, J=6.5 Hz, 3H), 1.05 (s, 9H). HRMS m/z [M+H]⁺ calcd for C₅₁H₆₄N₁₁O₈S⁺ 990.4655, found 990.4668.

### Example 97

### Synthesis of LQ076-138

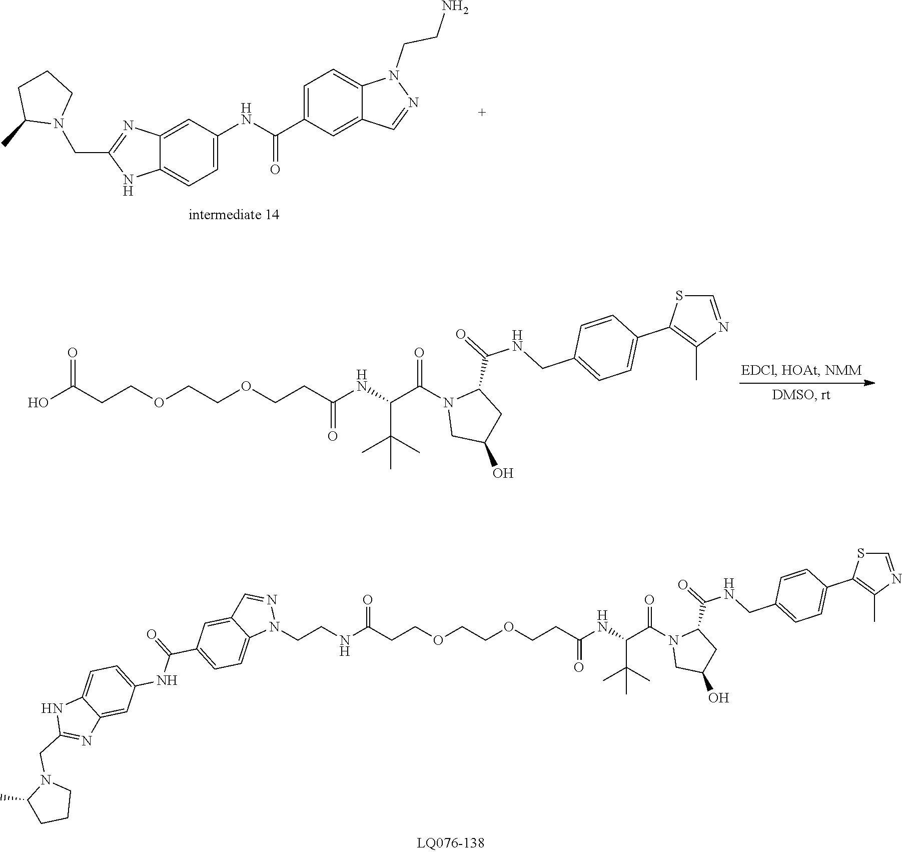 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)CCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCC(=O)NCCn2ncc3cc(C(=O)Nc4ccc5[nH]c(CN6CCC[C@@H]6C)nc5c4)ccc32)C(C)(C)C)cc1]

LQ076-138 was synthesized following the standard procedure for preparing LQ076-135 from intermediate 14 (13 mg, 0.02 mmol), 3-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)propanoic acid (12.6 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-138 was obtained as white solid in TFA salt form (17.1 mg, 69%). ¹H NMR (800 MHz, Methanol-d₄) δ 9.01 (s, 1H), 8.48 (s, 1H), 8.34 (s, 1H), 8.23 (s, 1H), 8.06 (d, J=8.8 Hz, 1H), 7.71-7.67 (m, 2H), 7.60 (d, J=8.7 Hz, 1H), 7.51-7.39 (m, 4H), 4.85 (d, J=14.7 Hz, 1H), 4.68-4.65 (m, 1H), 4.63-4.50 (m, 6H), 4.40-4.36 (m, 1H), 3.93-3.89 (m, 1H), 3.84-3.80 (m, 1H), 3.78-3.66 (m, 6H), 3.64-3.57 (m, 3H), 3.56-3.44 (m, 4H), 2.61-2.38 (m, 6H), 2.32 (t, J=6.3 Hz, 2H), 2.26-2.22 (m, 1H), 2.18-2.07 (m, 3H), 1.86-1.80 (m, 1H), 1.52 (d, J=6.5 Hz, 3H), 1.04 (s, 9H). HRMS m/z [M+H]⁺ calcd for C₅₃H₆₈N₁₁O₈S⁺ 1018.4968, found 1018.4990.

### Example 98

### Synthesis of LQ076-139

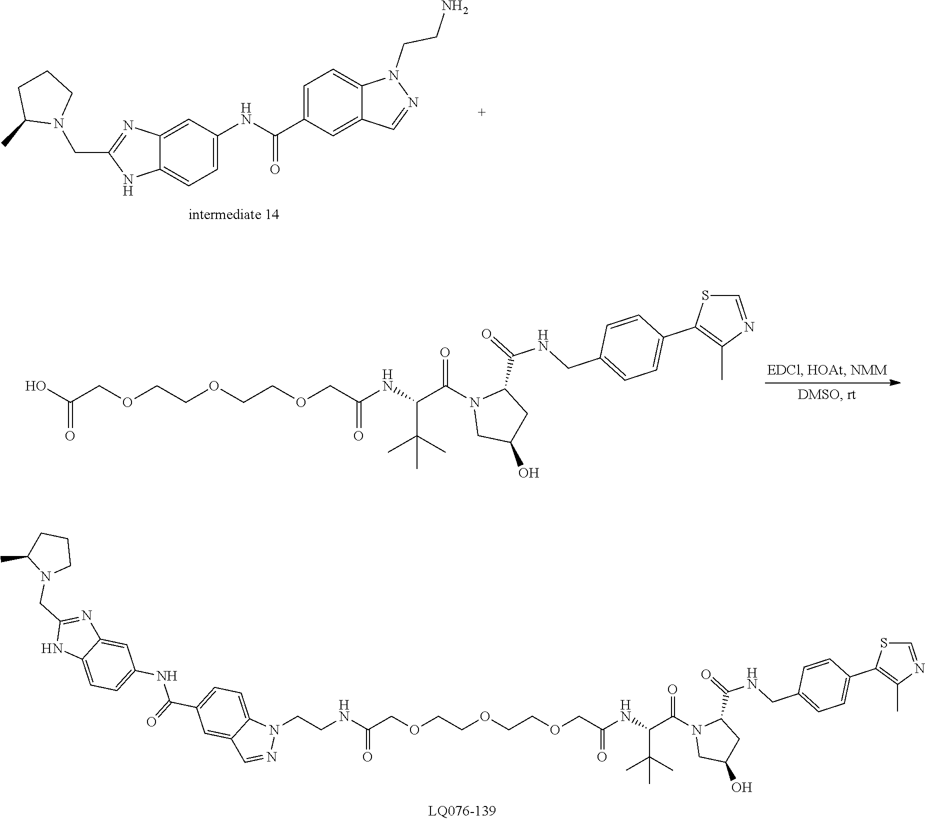 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)COCCOCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCCOCC(=O)NCCn2ncc3cc(C(=O)Nc4ccc5[nH]c(CN6CCC[C@@H]6C)nc5c4)ccc32)C(C)(C)C)cc1]

LQ076-139 was synthesized following the standard procedure for preparing LQ076-135 from intermediate 14 (13 mg, 0.02 mmol), (S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecanoic acid (13 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-139 was obtained as white solid in TFA salt form (16.5 mg, 65%). ¹H NMR (800 MHz, Methanol-d₄) δ 8.98 (s, 1H), 8.47 (s, 1H), 8.32 (s, 1H), 8.22 (s, 1H), 8.05 (d, J=8.7 Hz, 1H), 7.71-7.67 (m, 2H), 7.59 (d, J=8.7 Hz, 1H), 7.48-7.39 (m, 4H), 4.83 (d, J=14.7 Hz, 1H), 4.73-4.70 (m, 1H), 4.67-4.50 (m, 6H), 4.35 (d, J=15.2 Hz, 1H), 4.07-3.97 (m, 2H), 3.91-3.71 (m, 8H), 3.68-3.55 (m, 6H), 3.53-3.44 (m, 3H), 2.47 (s, 3H), 2.43-2.38 (m, 1H), 2.25 (dd, J=13.1, 7.6 Hz, 1H), 2.18-2.08 (m, 3H), 1.86-1.80 (m, 1H), 1.52 (d, J=6.5 Hz, 3H), 1.04 (s, 9H). HRMS m/z [M+H]⁺ calcd for C₅₃H₆₈N₁₁O₉S⁺ 1034.4917, found 1034.4919.

### Example 99

### Synthesis of LQ076-140

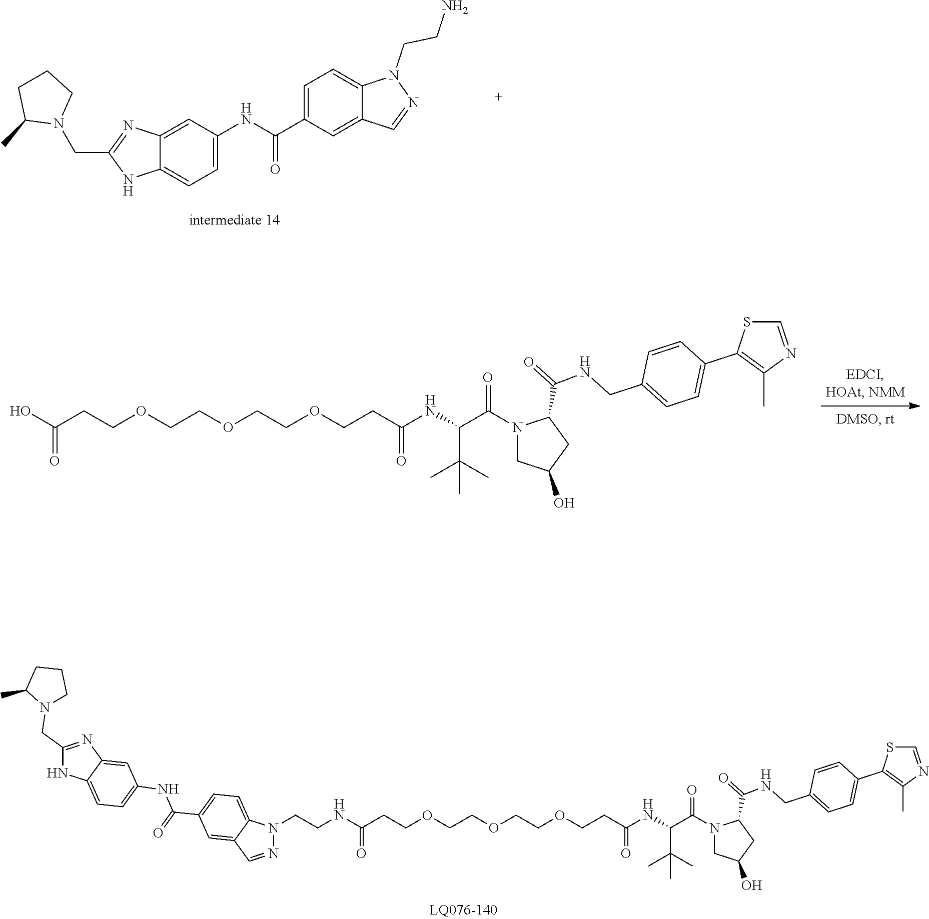 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)CCOCCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCOCCC(=O)NCCn2ncc3cc(C(=O)Nc4ccc5[nH]c(CN6CCC[C@@H]6C)nc5c4)ccc32)C(C)(C)C)cc1]

LQ076-140 was synthesized following the standard procedure for preparing LQ076-135 from intermediate 14 (13 mg, 0.02 mmol), (S)-15-((2S,4R)-4-hydroxy-2-((4-(4-methylthiazol-5-yl)benzyl)carbamoyl)pyrrolidine-1-carbonyl)-16,16-dimethyl-13-oxo-4,7,10-trioxa-14-azaheptadecanoic acid (13.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-140 was obtained as white solid in TFA salt form (18.2 mg, 71%). ¹H NMR (800 MHz, Methanol-d₄) δ 8.98 (s, 1H), 8.48 (s, 1H), 8.32 (s, 1H), 8.23 (s, 1H), 8.06 (d, J=8.8 Hz, 1H), 7.71-7.66 (m, 2H), 7.59 (d, J=8.7 Hz, 1H), 7.49-7.38 (m, 4H), 4.83 (d, J=14.7 Hz, 1H), 4.67-4.49 (m, 7H), 4.37 (d, J=15.4 Hz, 1H), 3.90 (d, J=10.9 Hz, 1H), 3.82-3.65 (m, 7H), 3.62-3.44 (m, 11H), 2.57-2.52 (m, 1H), 2.50-2.38 (m, 5H), 2.32 (t, J=6.2 Hz, 2H), 2.26-2.22 (m, 1H), 2.18-2.07 (m, 3H), 1.86-1.80 (m, 1H), 1.52 (d, J=6.5 Hz, 3H), 1.04 (s, 9H). HRMS m/z [M+H]⁺ calcd for C₅₅H₇₂N₁₁O₉S⁺ 1062.5230, found 1062.5218.

### Example 100

### Synthesis of LQ076-141

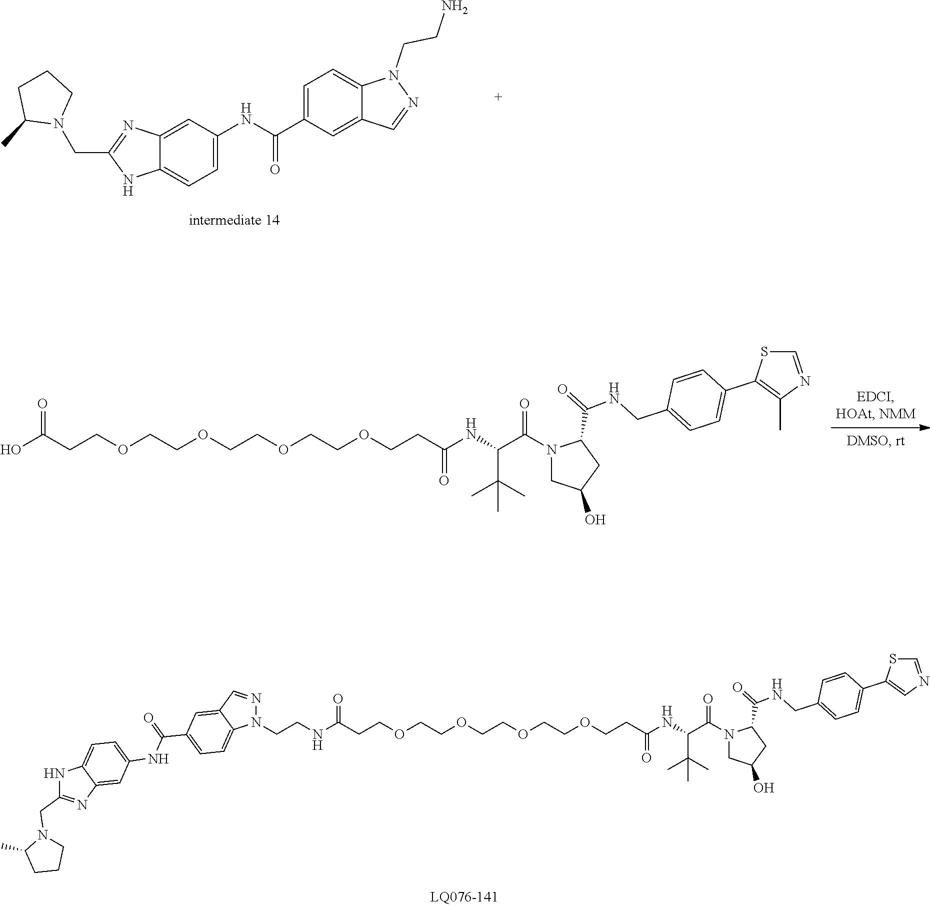 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)CCOCCOCCOCCOCCC(=O)O)C(C)(C)C)cc1 ; C[C@H]1CCCN1Cc1nc2cc(NC(=O)c3ccc4c(cnn4CCNC(=O)CCOCCOCCOCCOCCC(=O)N[C@H](C(=O)N4C[C@H](O)C[C@H]4C(=O)NCc4ccc(-c5cncs5)cc4)C(C)(C)C)c3)ccc2[nH]1]

LQ076-141 was synthesized following the standard procedure for preparing LQ076-135 from intermediate 14 (13 mg, 0.02 mmol), (S)-18-((2S,4R)-4-hydroxy-2-((4-(4-methylthiazol-5-yl)benzyl)carbamoyl)pyrrolidine-1-carbonyl)-19,19-dimethyl-16-oxo-4,7,10,13-tetraoxa-17-azaicosanoic acid (14.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-141 was obtained as white solid in TFA salt form (20 mg, 75%). ¹H NMR (800 MHz, Methanol-d₄) δ 9.00 (s, 1H), 8.49 (s, 1H), 8.32 (s, 1H), 8.24 (s, 1H), 8.06 (d, J=8.8 Hz, 1H), 7.72-7.67 (m, 2H), 7.60 (d, J=8.7 Hz, 1H), 7.51-7.39 (m, 4H), 4.83 (d, J=14.7 Hz, 1H), 4.67-4.50 (m, 7H), 4.37 (d, J=15.4 Hz, 1H), 3.90 (d, J=11.0 Hz, 1H), 3.81 (dd, J=11.0, 3.9 Hz, 1H), 3.78-3.65 (m, 6H), 3.63-3.44 (m, 15H), 2.58-2.53 (m, 1H), 2.50-2.38 (m, 5H), 2.33 (t, J=6.2 Hz, 2H), 2.26-2.22 (m, 1H), 2.19-2.07 (m, 3H), 1.86-1.80 (m, 1H), 1.52 (d, J=6.6 Hz, 3H), 1.04 (s, 9H). HRMS m/z [M+H]⁺ calcd for C₅₇H₇₆N₁₁O₁₀S⁺ 1106.5492, found 1106.5511.

### Example 101

### Synthesis of LQ076-142

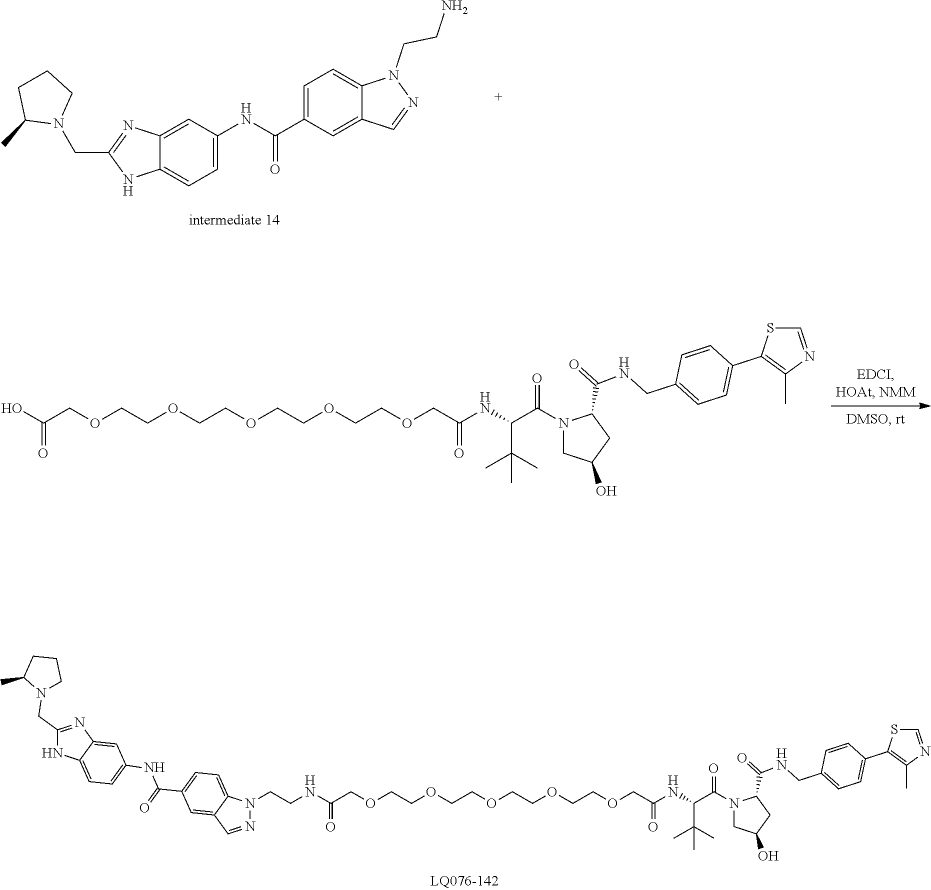 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)COCCOCCOCCOCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCCOCCOCCOCC(=O)NCCn2ncc3cc(C(=O)Nc4ccc5[nH]c(CN6CCC[C@@H]6C)nc5c4)ccc32)C(C)(C)C)cc1]

LQ076-142 was synthesized following the standard procedure for preparing LQ076-135 from intermediate 14 (13 mg, 0.02 mmol), (S)-19-((2S,4R)-4-hydroxy-2-((4-(4-methylthiazol-5-yl)benzyl)carbamoyl)pyrrolidine-1-carbonyl)-20,20-dimethyl-17-oxo-3,6,9,12,15-pentaoxa-18-azahenicosanoic acid (15.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-142 was obtained as white solid in TFA salt form (20.2 mg, 75%). ¹H NMR (800 MHz, Methanol-d₄) δ 8.99 (s, 1H), 8.48 (s, 1H), 8.32 (s, 1H), 8.22 (s, 1H), 8.05 (d, J=8.8 Hz, 1H), 7.73-7.68 (m, 2H), 7.60 (d, J=8.7 Hz, 1H), 7.49-7.39 (m, 4H), 4.83 (d, J=14.7 Hz, 1H), 4.71-4.69 (m, 1H), 4.67-4.51 (m, 6H), 4.37 (d, J=15.4 Hz, 1H), 4.05-3.97 (m, 2H), 3.89 (d, J=11.0 Hz, 1H), 3.85-3.80 (m, 3H), 3.79-3.71 (m, 4H), 3.69-3.61 (m, 8H), 3.58-3.44 (m, 9H), 2.48 (s, 3H), 2.43-2.38 (m, 1H), 2.27-2.23 (m, 1H), 2.18-2.08 (m, 3H), 1.86-1.80 (m, 1H), 1.52 (d, J=6.5 Hz, 3H), 1.05 (s, 9H). HRMS m/z [M+H]⁺ calcd for C₅₇H₇₆N₁₁O₁₁S⁺ 1122.5441, found 1122.5440.

### Example 102

### Synthesis of LQ076-143

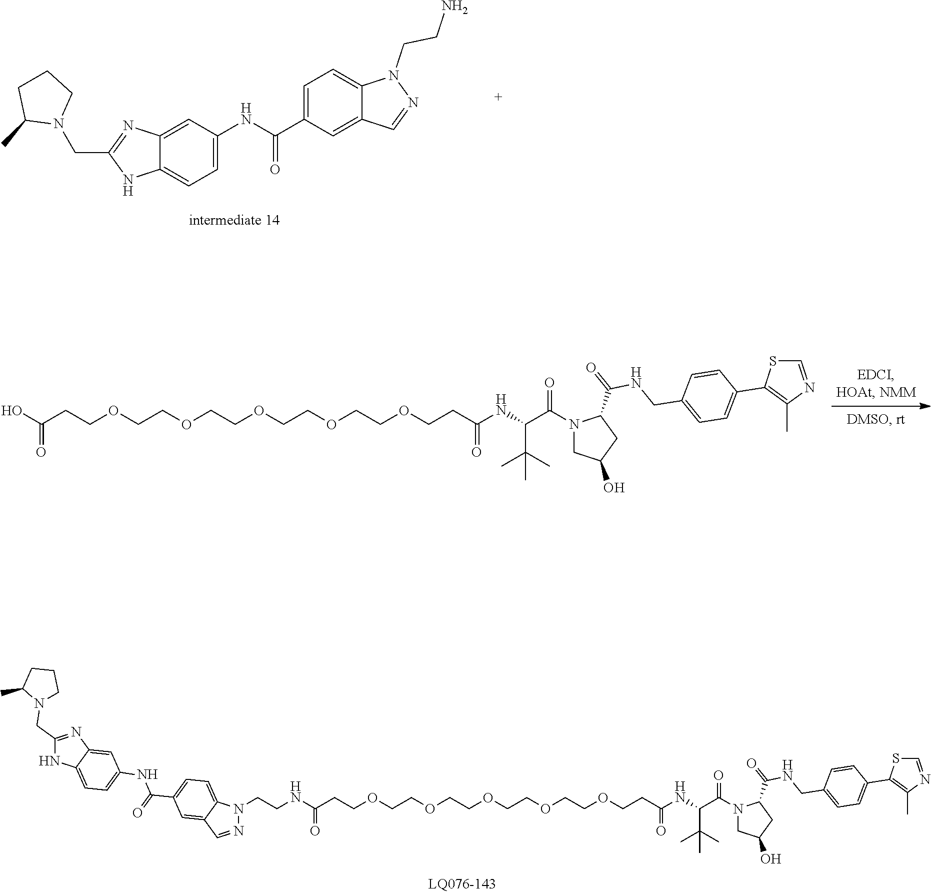 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)CCOCCOCCOCCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCOCCOCCOCCC(=O)NCCn2ncc3cc(C(=O)Nc4ccc5[nH]c(CN6CCC[C@@H]6C)nc5c4)ccc32)C(C)(C)C)cc1]

LQ076-143 was synthesized following the standard procedure for preparing LQ076-135 from intermediate 14 (13 mg, 0.02 mmol), (S)-21-((2S,4R)-4-hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azatricosanoic acid (15.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-143 was obtained as white solid in TFA salt form (19.5 mg, 70%). ¹H NMR (800 MHz, Methanol-d₄) δ 8.99 (s, 1H), 8.49 (s, 1H), 8.32 (s, 1H), 8.24 (s, 1H), 8.06 (d, J=8.8 Hz, 1H), 7.73-7.67 (m, 2H), 7.60 (d, J=8.7 Hz, 1H), 7.50-7.38 (m, 4H), 4.83 (d, J=14.6 Hz, 1H), 4.67-4.65 (m, 1H), 4.63-4.49 (m, 6H), 4.37 (d, J=15.4 Hz, 1H), 3.90 (d, J=11.0 Hz, 1H), 3.81 (dd, J=10.9, 3.9 Hz, 1H), 3.78-3.44 (m, 25H), 2.58-2.54 (m, 1H), 2.50-2.44 (m, 4H), 2.43-2.38 (m, 1H), 2.33 (t, J=6.2 Hz, 2H), 2.26-2.22 (m, 1H), 2.19-2.07 (m, 3H), 1.87-1.81 (m, 1H), 1.52 (d, J=6.5 Hz, 3H), 1.05 (s, 9H). HRMS m/z [M+H]⁺ calcd for C₅₉H₈₀N₁₁O₁₁S⁺ 1150.5754, found 1150.5782.

### Example 103

### Synthesis of LQ076-144

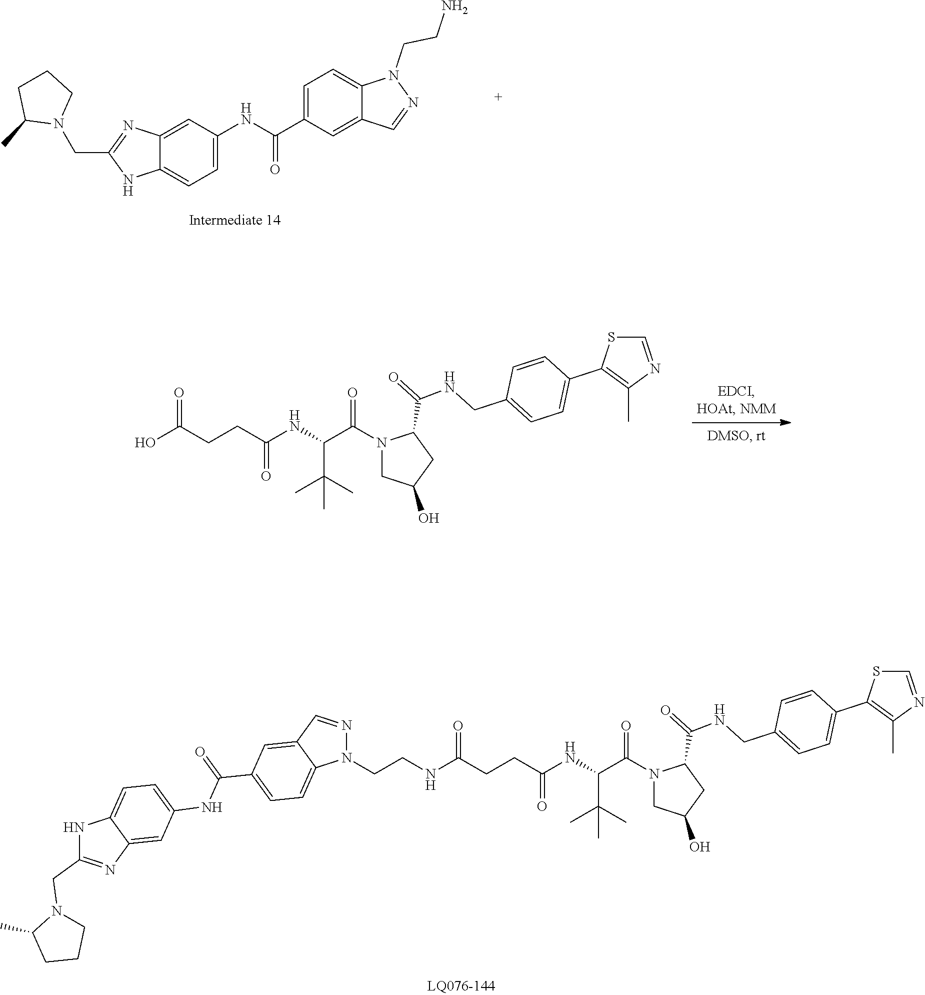 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)CCC(=O)O)C(C)(C)C)cc1 ; Cc1ncsc1-c1ccc(CNC(=O)[C@@H]2C[C@@H](O)CN2C(=O)[C@@H](NC(=O)CCC(=O)NCCn2ncc3cc(C(=O)Nc4ccc5[nH]c(CN6CCC[C@@H]6C)nc5c4)ccc32)C(C)(C)C)cc1]

LQ076-144 was synthesized following the standard procedure for preparing LQ076-135 from intermediate 14 (13 mg, 0.02 mmol), 4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-4-oxobutanoic acid (10.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-144 was obtained as white solid in TFA salt form (14.6 mg, 63%). ¹H NMR (800 MHz, Methanol-d₄) δ 8.99 (s, 1H), 8.47 (s, 1H), 8.32 (s, 1H), 8.23 (s, 1H), 8.06 (d, J=8.9 Hz, 1H), 7.71-7.65 (m, 2H), 7.60 (d, J=8.6 Hz, 1H), 7.49-7.37 (m, 4H), 4.82 (d, J=14.7 Hz, 1H), 4.63-4.47 (m, 7H), 4.37 (d, J=15.6 Hz, 1H), 3.92 (d, J=10.9 Hz, 1H), 3.81 (dd, J=10.9, 3.9 Hz, 1H), 3.74 (s, 2H), 3.71-3.63 (m, 2H), 3.49-3.44 (m, 1H), 2.53-2.32 (m, 8H), 2.26-2.21 (m, 1H), 2.18-2.06 (m, 3H), 1.86-1.81 (m, 1H), 1.52 (d, J=6.6 Hz, 3H), 1.05 (s, 9H). HRMS m/z [M+H]⁺ calcd for C₄₉H₆₀N₁₁O₆S⁺ 930.4443, found 930.4458.

### Example 104

### Synthesis of LQ076-145

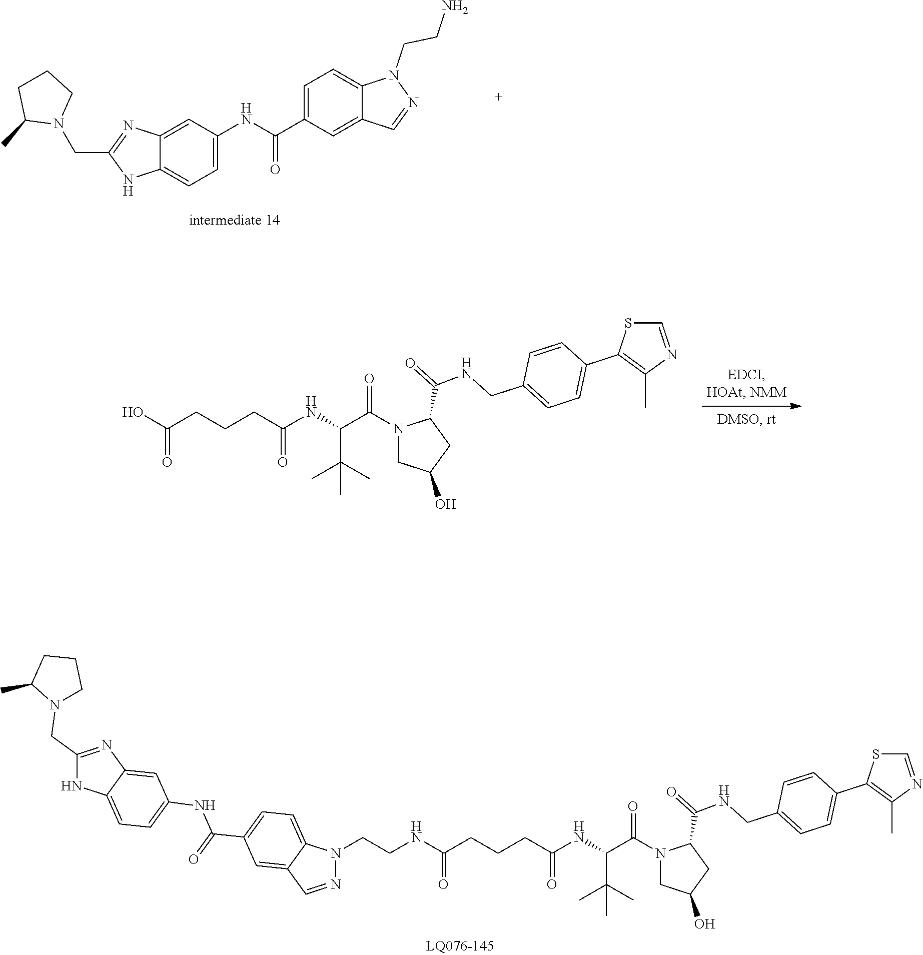 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)CCCC(=O)O)C(C)(C)C)cc1 ; Cc1ncsc1-c1ccc(CNC(=O)[C@@H]2C[C@@H](O)CN2C(=O)[C@@H](NC(=O)CCCC(=O)NCCn2ncc3cc(C(=O)Nc4ccc5[nH]c(CN6CCC[C@@H]6C)nc5c4)ccc32)C(C)(C)C)cc1]

LQ076-145 was synthesized following the standard procedure for preparing LQ076-135 from intermediate 14 (13 mg, 0.02 mmol), 5-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-5-oxopentanoic acid (11.6 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-145 was obtained as white solid in TFA salt form (17.1 mg, 73%). ¹H NMR (800 MHz, Methanol-d₄) δ 8.96 (s, 1H), 8.47 (s, 1H), 8.32 (s, 1H), 8.23 (s, 1H), 8.05 (d, J=8.8 Hz, 1H), 7.70-7.66 (m, 2H), 7.60 (d, J=8.7 Hz, 1H), 7.47 (d, J=7.8 Hz, 2H), 7.40 (d, J=7.9 Hz, 2H), 4.81 (d, J=14.6 Hz, 1H), 4.64-4.49 (m, 7H), 4.36 (d, J=15.4 Hz, 1H), 3.92 (d, J=11.0 Hz, 1H), 3.80-3.63 (m, 5H), 3.49-3.44 (m, 1H), 2.47 (s, 3H), 2.43-2.36 (m, 1H), 2.26-2.05 (m, 8H), 1.86-1.80 (m, 1H), 1.78-1.73 (m, 2H), 1.51 (d, J=6.5 Hz, 3H), 1.04 (s, 9H). HRMS m/z [M+H]⁺ calcd for C₅₀H₆₂N₁₁O₆S⁺ 944.4600, found 944.4622.

### Example 105

### Synthesis of LQ076-146

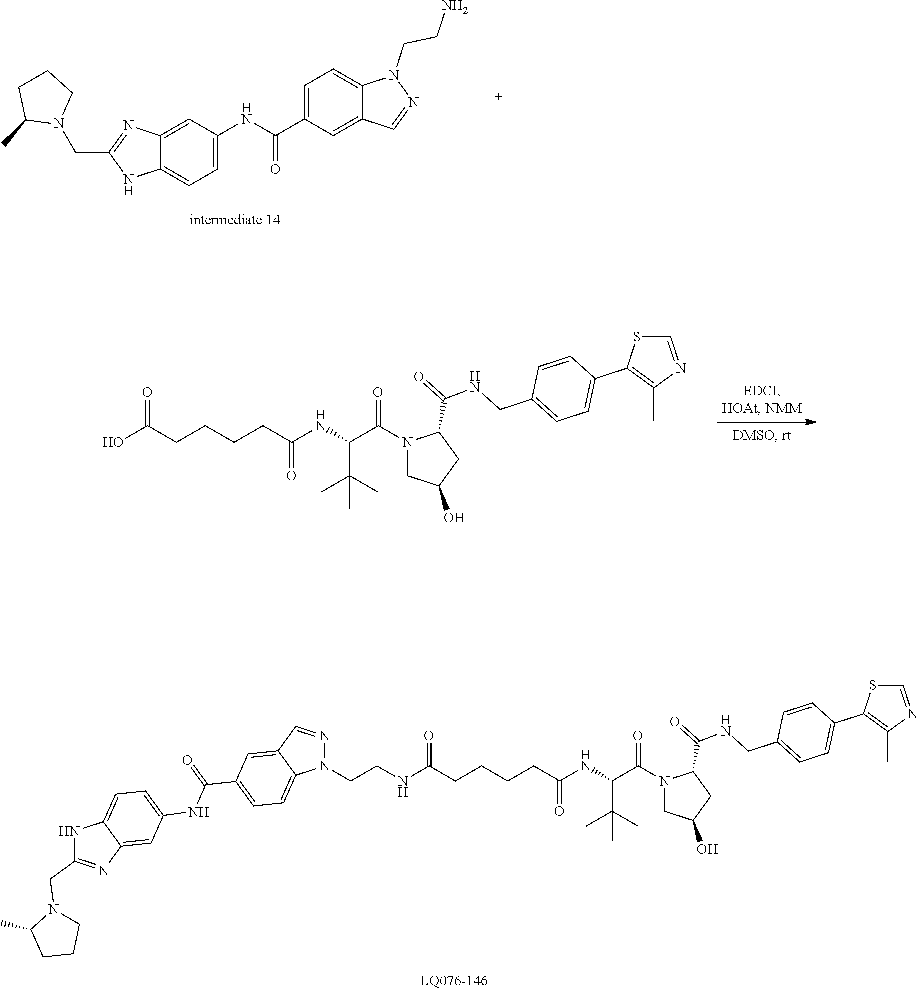 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)CCCCC(=O)O)C(C)(C)C)cc1 ; Cc1ncsc1-c1ccc(CNC(=O)[C@@H]2C[C@@H](O)CN2C(=O)[C@@H](NC(=O)CCCCC(=O)NCCn2ncc3cc(C(=O)Nc4ccc5[nH]c(CN6CCC[C@@H]6C)nc5c4)ccc32)C(C)(C)C)cc1]

LQ076-146 was synthesized following the standard procedure for preparing LQ076-135 from intermediate 14 (13 mg, 0.02 mmol), 6-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-6-oxohexanoic acid (11.4 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-146 was obtained as white solid in TFA salt form (15.5 mg, 65%). ¹H NMR (800 MHz, Methanol-d₄) δ 9.00 (s, 1H), 8.47 (s, 1H), 8.34 (s, 1H), 8.23 (s, 1H), 8.05 (d, J=8.8 Hz, 1H), 7.70-7.66 (m, 2H), 7.61 (d, J=8.7 Hz, 1H), 7.47 (d, J=7.8 Hz, 2H), 7.41 (d, J=7.9 Hz, 2H), 4.83 (d, J=14.7 Hz, 1H), 4.65-4.49 (m, 7H), 4.38 (d, J=15.4 Hz, 1H), 3.93 (d, J=11.0 Hz, 1H), 3.82-3.64 (m, 5H), 3.49-3.44 (m, 1H), 2.48 (s, 3H), 2.43-2.38 (m, 1H), 2.26-2.03 (m, 8H), 1.86-1.80 (m, 1H), 1.53-1.42 (m, 7H), 1.05 (s, 9H). HRMS m/z [M+H]⁺ calcd for C₅₁H₆₄N₁₁O₆S⁺ 958.4756, found 958.4755.

### Example 106

### Synthesis of LQ076-147

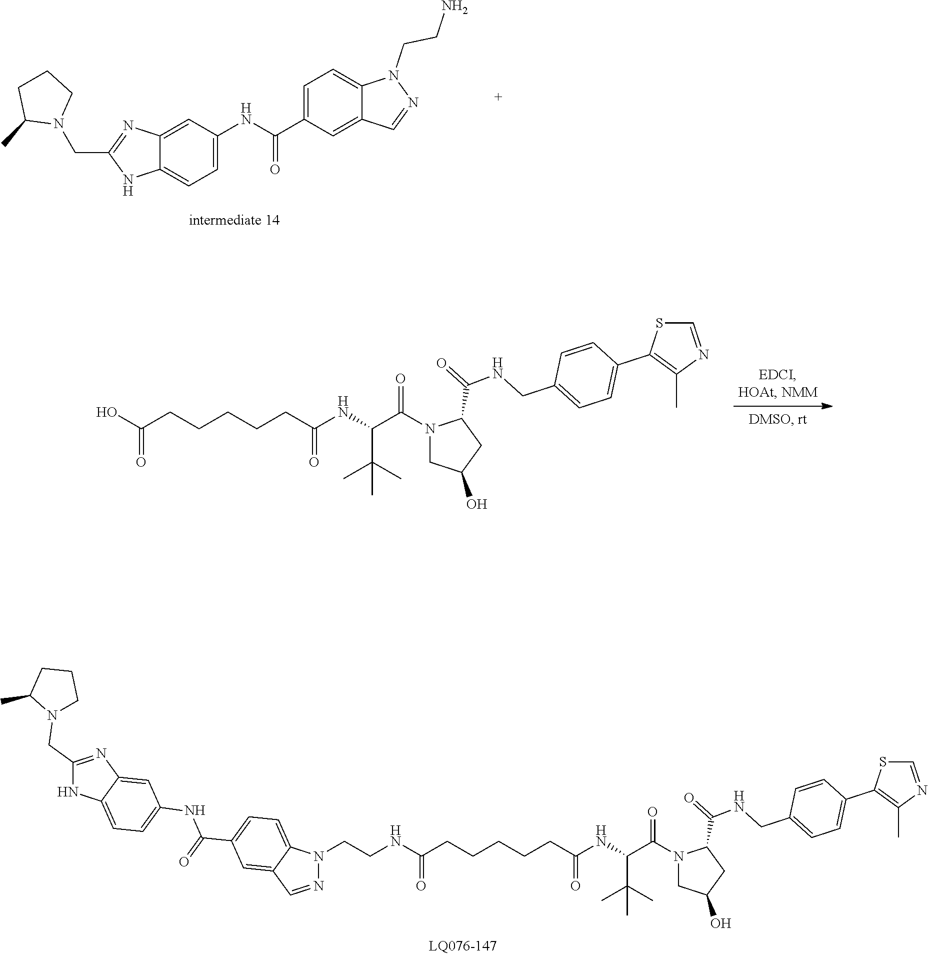 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)CCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCC(=O)NCCn2ncc3cc(C(=O)Nc4ccc5[nH]c(CN6CCC[C@@H]6C)nc5c4)ccc32)C(C)(C)C)cc1]

LQ076-147 was synthesized following the standard procedure for preparing LQ076-135 from intermediate 14 (13 mg, 0.02 mmol), 7-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-7-oxoheptanoic acid (12.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-147 was obtained as white solid in TFA salt form (17.3 mg, 72%). ¹H NMR (800 MHz, Methanol-d₄) δ 8.97 (s, 1H), 8.48 (s, 1H), 8.34 (s, 1H), 8.23 (s, 1H), 8.05 (d, J=8.8 Hz, 1H), 7.70-7.66 (m, 2H), 7.59 (d, J=8.6 Hz, 1H), 7.48 (d, J=7.8 Hz, 2H), 7.41 (d, J=7.8 Hz, 2H), 4.81 (d, J=14.8 Hz, 1H), 4.65-4.49 (m, 7H), 4.38 (d, J=15.4 Hz, 1H), 3.91 (d, J=11.0 Hz, 1H), 3.80 (dd, J=10.9, 3.9 Hz, 1H), 3.77-3.68 (m, 4H), 3.49-3.43 (m, 1H), 2.48 (s, 3H), 2.42-2.37 (m, 1H), 2.28-2.02 (m, 8H), 1.86-1.80 (m, 1H), 1.59-1.42 (m, 7H), 1.23-1.17 (m, 2H), 1.04 (s, 9H). HRMS m/z [M+H]⁺ calcd for C₅₂H₆₆N₁₁O₆S⁺ 972.4913, found 972.4936.

### Example 107

### Synthesis of LQ076-148

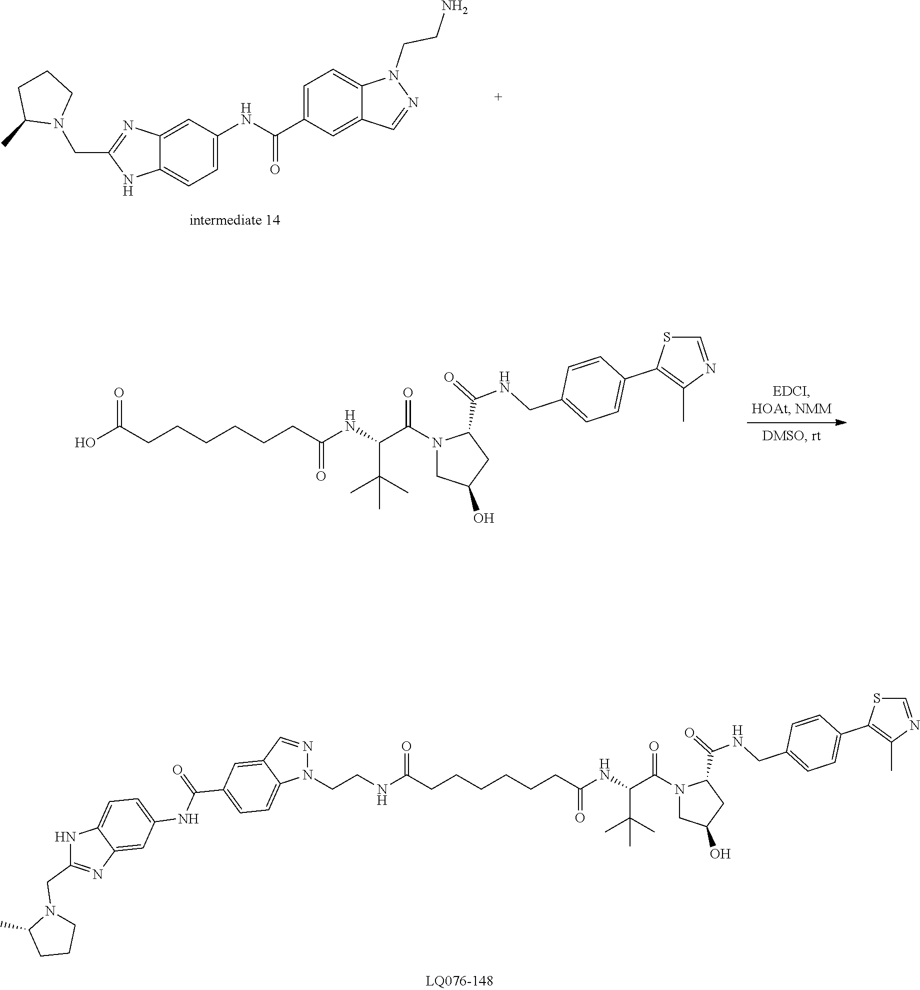 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)CCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCC(=O)NCCn2ncc3cc(C(=O)Nc4ccc5[nH]c(CN6CCC[C@@H]6C)nc5c4)ccc32)C(C)(C)C)cc1]

LQ076-148 was synthesized following the standard procedure for preparing LQ076-135 from intermediate 14 (13 mg, 0.02 mmol), 8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctanoic acid (12.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-148 was obtained as white solid in TFA salt form (16.7 mg, 69%). ¹H NMR (800 MHz, Methanol-d₄) δ 9.00 (s, 1H), 8.50 (s, 1H), 8.35 (s, 1H), 8.23 (s, 1H), 8.06 (d, J=8.8 Hz, 1H), 7.71-7.65 (m, 2H), 7.61 (d, J=8.5 Hz, 1H), 7.48 (d, J=7.8 Hz, 2H), 7.41 (d, J=7.8 Hz, 2H), 4.84 (d, J=14.7 Hz, 1H), 4.66-4.50 (m, 7H), 4.38 (d, J=15.5 Hz, 1H), 3.93 (d, J=11.0 Hz, 1H), 3.81 (dd, J=10.9, 4.0 Hz, 1H), 3.78-3.66 (m, 4H), 3.49-3.44 (m, 1H), 2.47 (s, 3H), 2.43-2.38 (m, 1H), 2.29-2.20 (m, 3H), 2.18-2.07 (m, 3H), 2.03 (t, J=7.5 Hz, 2H), 1.86-1.80 (m, 1H), 1.56-1.49 (m, 5H), 1.43-1.38 (m, 2H), 1.26-1.20 (m, 2H), 1.19-1.13 (m, 2H), 1.05 (s, 9H). HRMS m/z [M+H]⁺ calcd for C₅₃H₆₈N₁₁O₆S⁺ 986.5069, found 986.5060.

### Example 108

### Synthesis of LQ076-149

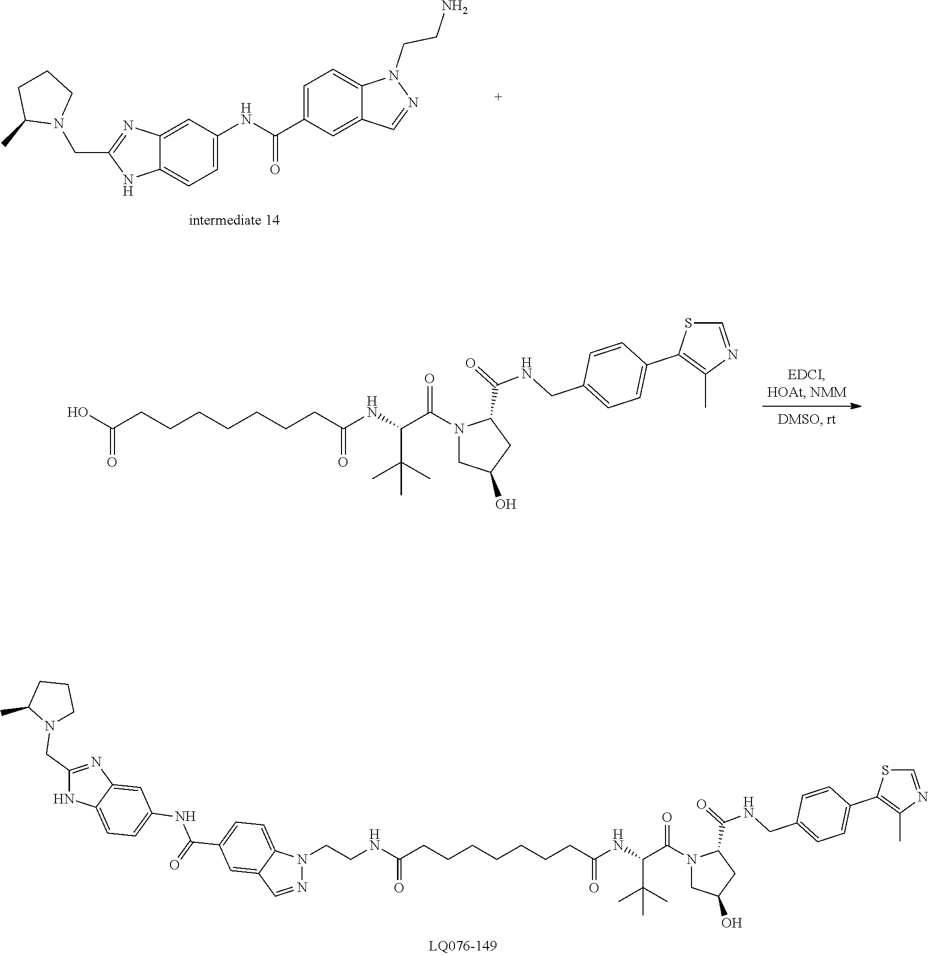 [SMILES: C[C@H]1CCCN1Cc1nc2cc(NC(=O)c3ccc4c(cnn4CCN)c3)ccc2[nH]1 ; Cc1ncsc1-c1ccc(CNC(=O)[C@@H]2C[C@@H](O)CN2C(=O)[C@@H](NC(=O)CCCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCCC(=O)NCCn2ncc3cc(C(=O)Nc4ccc5[nH]c(CN6CCC[C@@H]6C)nc5c4)ccc32)C(C)(C)C)cc1]

LQ076-149 was synthesized following the standard procedure for preparing LQ076-135 from intermediate 14 (13 mg, 0.02 mmol), 9-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-9-oxononanoic acid (13.1 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-149 was obtained as white solid in TFA salt form (17.7 mg, 72%). ¹H NMR (800 MHz, Methanol-d₄) δ 9.00 (s, 1H), 8.50 (s, 1H), 8.34 (s, 1H), 8.23 (s, 1H), 8.06 (d, J=8.8 Hz, 1H), 7.71-7.65 (m, 2H), 7.60 (d, J=8.7 Hz, 1H), 7.48 (d, J=7.8 Hz, 2H), 7.42 (d, J=7.8 Hz, 2H), 4.83 (d, J=14.6 Hz, 1H), 4.66-4.64 (m, 1H), 4.62-4.49 (m, 6H), 4.39 (d, J=15.5 Hz, 1H), 3.93 (d, J=10.9 Hz, 1H), 3.82 (dd, J=10.9, 4.0 Hz, 1H), 3.78-3.65 (m, 4H), 3.49-3.44 (m, 1H), 2.48 (s, 3H), 2.43-2.37 (m, 1H), 2.30-2.20 (m, 3H), 2.18-2.07 (m, 3H), 2.03 (t, J=7.6 Hz, 2H), 1.86-1.80 (m, 1H), 1.57 (d, J=6.9 Hz, 2H), 1.51 (d, J=6.5 Hz, 3H), 1.44-1.38 (m, 2H), 1.28-1.20 (m, 4H), 1.18-1.12 (m, 2H), 1.05 (s, 9H). HRMS m/z [M+H]⁺ calcd for C₅₄H₇₀N₁₁O₆S⁺ 1000.5226, found 1000.5273.

### Example 109

### Synthesis of LQ076-150

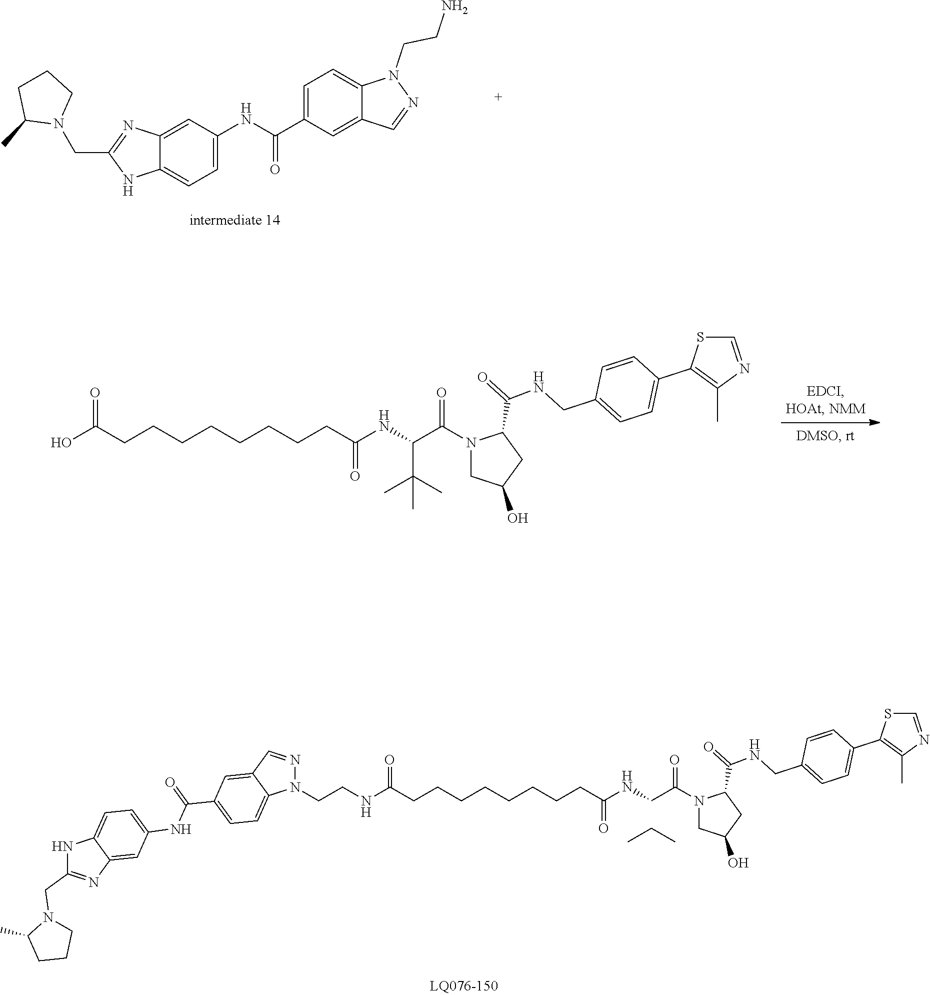

LQ076-150 was synthesized following the standard procedure for preparing LQ076-135 from intermediate 14 (13 mg, 0.02 mmol), 10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecanoic acid (13.6 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-150 was obtained as white solid in TFA salt form (14.8 mg, 59%). ¹H NMR (800 MHz, Methanol-d₄) δ 8.98 (s, 1H), 8.50 (s, 1H), 8.33 (s, 1H), 8.23 (s, 1H), 8.06 (d, J=8.8 Hz, 1H), 7.70-7.66 (m, 2H), 7.59 (d, J=8.7 Hz, 1H), 7.48 (d, J=7.8 Hz, 2H), 7.42 (d, J=7.8 Hz, 2H), 4.82 (d, J=14.6 Hz, 1H), 4.66-4.64 (m, 1H), 4.62-4.50 (m, 6H), 4.39 (d, J=15.3 Hz, 1H), 3.92 (d, J=10.9 Hz, 1H), 3.82 (dd, J=10.9, 4.0 Hz, 1H), 3.77-3.67 (m, 4H), 3.49-3.44 (m, 1H), 2.48 (s, 3H), 2.42-2.38 (m, 1H), 2.29-2.08 (m, 6H), 2.02 (t, J=7.6 Hz, 2H), 1.86-1.80 (m, 1H), 1.61-1.54 (m, 2H), 1.51 (d, J=6.5 Hz, 3H), 1.42-1.37 (m, 2H), 1.31-1.19 (m, 6H), 1.18-1.11 (m, 2H), 1.05 (s, 9H). HRMS m/z [M+H]⁺ calcd for C₅₅H₇₂N₁₁O₆S⁺ 1014.5382, found 1014.5381.

### Example 110

### Synthesis of LQ076-151

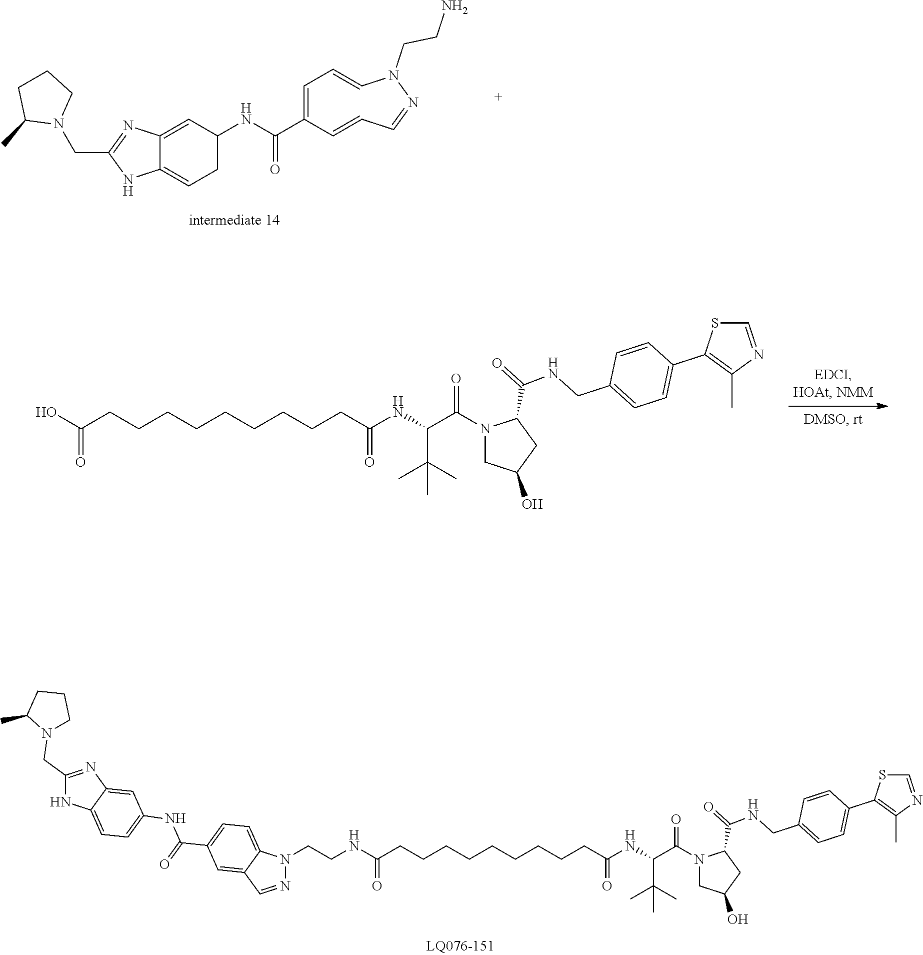 [SMILES: C[C@H]1CCCN1Cc1nc2c([nH]1)=CCC(NC(=O)c1cccnn(CCN)ccc1)C=2 ; Cc1ncsc1-c1ccc(CNC(=O)[C@@H]2C[C@@H](O)CN2C(=O)[C@@H](NC(=O)CCCCCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCCCCC(=O)NCCn2ncc3cc(C(=O)Nc4ccc5[nH]c(CN6CCC[C@@H]6C)nc5c4)ccc32)C(C)(C)C)cc1]

LQ076-151 was synthesized following the standard procedure for preparing LQ076-135 from intermediate 14 (13 mg, 0.02 mmol), 11-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-11-oxoundecanoic acid (13.6 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-151 was obtained as white solid in TFA salt form (18.9 mg, 75%). ¹H NMR (800 MHz, Methanol-d₄) δ 8.97 (s, 1H), 8.50 (s, 1H), 8.32 (s, 1H), 8.23 (s, 1H), 8.06 (d, J=8.8 Hz, 1H), 7.70-7.66 (m, 2H), 7.58 (d, J=8.7 Hz, 1H), 7.48 (d, J=7.8 Hz, 2H), 7.42 (d, J=7.8 Hz, 2H), 4.81 (d, J=14.6 Hz, 1H), 4.66-4.63 (m, 1H), 4.62-4.49 (m, 6H), 4.38 (d, J=15.4 Hz, 1H), 3.93 (d, J=10.9 Hz, 1H), 3.82 (dd, J=10.9, 4.0 Hz, 1H), 3.77-3.68 (m, 4H), 3.50-3.44 (m, 1H), 2.49 (s, 3H), 2.43-2.38 (m, 1H), 2.28-2.09 (m, 6H), 2.04-2.00 (m, 2H), 1.86-1.80 (m, 1H), 1.60-1.53 (m, 2H), 1.51 (d, J=6.5 Hz, 3H), 1.41-1.36 (m, 2H), 1.32-1.11 (m, 10H), 1.05 (s, 9H). HRMS m/z [M+H]⁺ calcd for C₅₆H₇₄N₁₁O₆S⁺ 1028.5539, found 1028.5529.

### Example 111

### Synthesis of LQ076-152

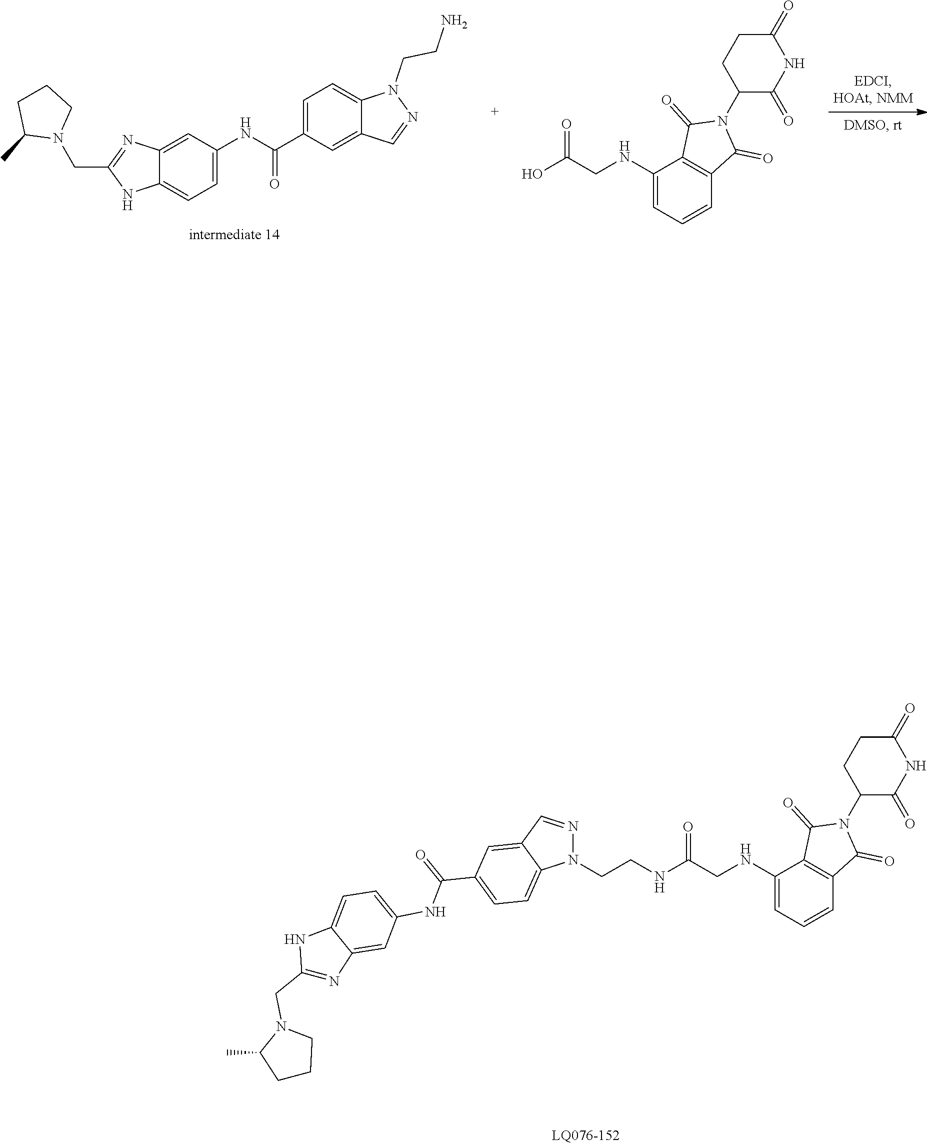 [SMILES: C[C@H]1CCCN1Cc1nc2cc(NC(=O)c3ccc4c(cnn4CCN)c3)ccc2[nH]1 ; O=C(O)CNc1cccc2c1C(=O)N(C1CCC(=O)NC1=O)C2=O ; C[C@H]1CCCN1Cc1nc2cc(NC(=O)c3ccc4c(cnn4CCNC(=O)CNc4cccc5c4C(=O)N(C4CCC(=O)NC4=O)C5=O)c3)ccc2[nH]1]

LQ076-152 was synthesized following the standard procedure for preparing LQ076-135 from intermediate 14 (13 mg, 0.02 mmol), (2-(2,6-dioxopiperidin-3-yl)-1,3-dioxoisoindolin-4-yl)glycine (6.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-152 was obtained as yellow solid in TFA salt form (11.2 mg, 58%). ¹H NMR (800 MHz, Methanol-d₄) δ 8.42 (s, 1H), 8.29 (s, 1H), 8.17 (s, 1H), 7.97 (d, J=8.8 Hz, 1H), 7.68 (d, J=8.7 Hz, 1H), 7.61 (d, J=8.8 Hz, 1H), 7.57 (d, J=8.7 Hz, 1H), 7.39 (t, J=7.8 Hz, 1H), 7.01 (d, J=7.1 Hz, 1H), 6.61 (d, J=8.5 Hz, 1H), 5.05 (dd, J=12.8, 5.6 Hz, 1H), 4.80 (d, J=14.6 Hz, 1H), 4.61-4.55 (m, 3H), 3.84 (s, 2H), 3.79-3.72 (m, 4H), 3.50-3.45 (m, 1H), 2.86-2.79 (m, 1H), 2.74-2.67 (m, 2H), 2.43-2.38 (m, 1H), 2.19-2.08 (m, 3H), 1.86-1.80 (m, 1H), 1.52 (d, J=6.5 Hz, 3H). HRMS m/z [M+H]⁺ calcd for C₃₈H₃₉N₁₀O₆⁺ 731.3049, found 731.3050.

### Example 112

### Synthesis of LQ076-153

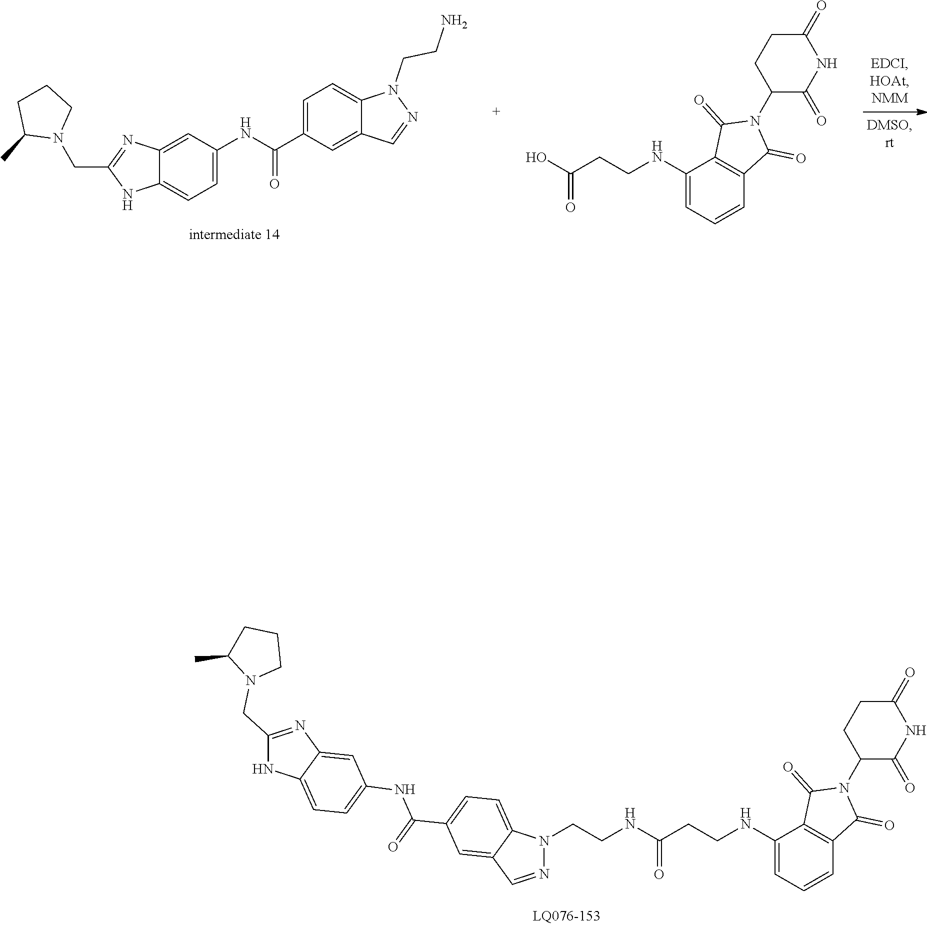 [SMILES: C[C@H]1CCCN1Cc1nc2cc(NC(=O)c3ccc4c(cnn4CCN)c3)ccc2[nH]1 ; O=C(O)CCNc1cccc2c1C(=O)N(C1CCC(=O)NC1=O)C2=O ; C[C@H]1CCCN1Cc1nc2cc(NC(=O)c3ccc4c(cnn4CCNC(=O)CCNc4cccc5c4C(=O)N(C4CCC(=O)NC4=O)C5=O)c3)ccc2[nH]1]

LQ076-153 was synthesized following the standard procedure for preparing LQ076-135 from intermediate 14 (13 mg, 0.02 mmol), 3-((2-(2,6-dioxopiperidin-3-yl)-1,3-dioxoisoindolin-4-yl)amino)propanoic acid (7.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-153 was obtained as yellow solid in TFA salt form (13.3 mg, 68%). ¹H NMR (800 MHz, Methanol-d₄) δ 8.40 (s, 1H), 8.27 (s, 1H), 8.18 (s, 1H), 7.92 (d, J=8.7 Hz, 1H), 7.68 (d, J=8.7 Hz, 1H), 7.61 (d, J=8.7 Hz, 1H), 7.55 (d, J=8.7 Hz, 1H), 7.50 (t, J=7.8 Hz, 1H), 6.98-6.94 (m, 2H), 5.06 (dd, J=12.8, 5.6 Hz, 1H), 4.81 (d, J=14.6 Hz, 1H), 4.61-4.54 (m, 3H), 3.78-3.67 (m, 4H), 3.50-3.45 (m, 1H), 3.41 (t, J=6.6 Hz, 2H), 2.86-2.80 (m, 1H), 2.74-2.67 (m, 2H), 2.43-2.33 (m, 3H), 2.19-2.07 (m, 3H), 1.86-1.80 (m, 1H), 1.52 (d, J=6.5 Hz, 3H). HRMS m/z [M+H]⁺ calcd for C₃₉H₄₁N₁₀O₆⁺ 745.3205, found 745.3204.

### Example 113

### Synthesis of LQ076-154

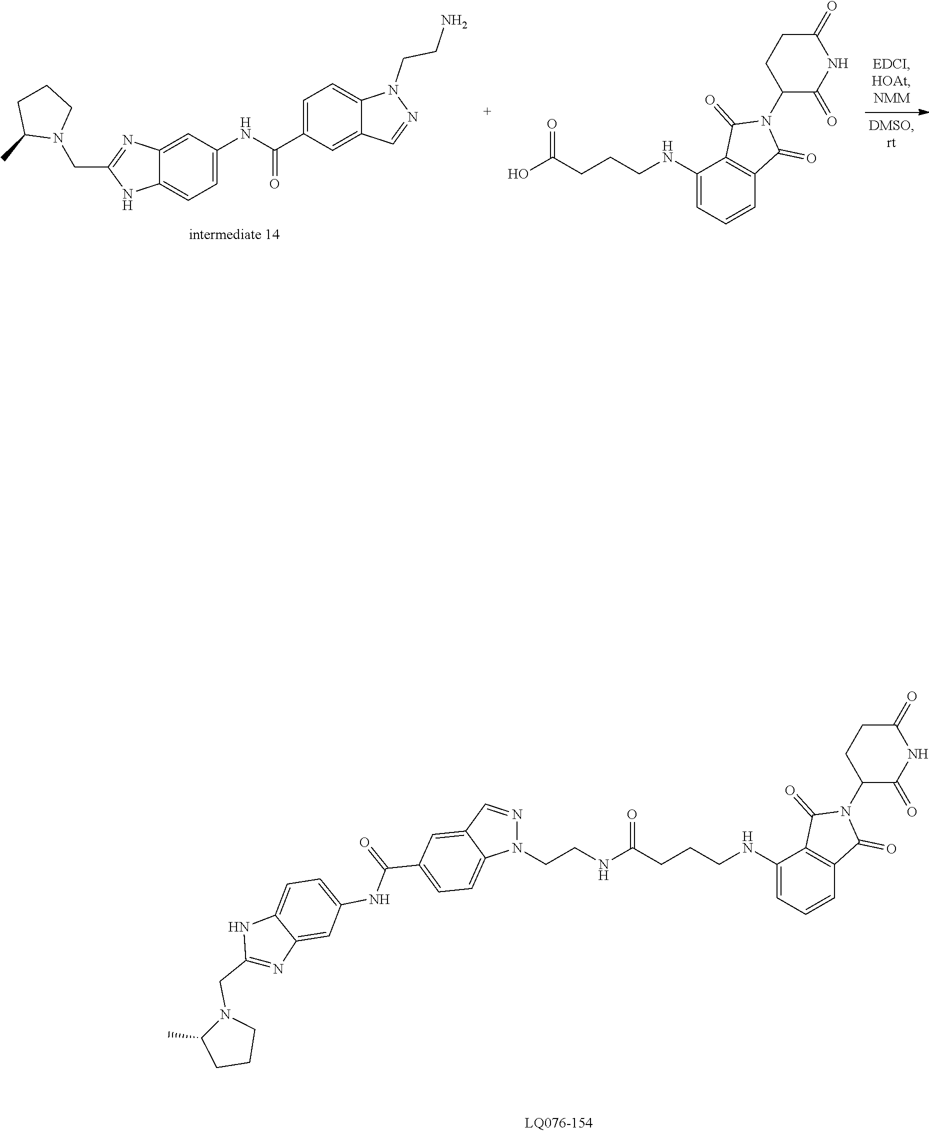 [SMILES: C[C@H]1CCCN1Cc1nc2cc(NC(=O)c3ccc4c(cnn4CCN)c3)ccc2[nH]1 ; O=C(O)CCCNc1cccc2c1C(=O)N(C1CCC(=O)NC1=O)C2=O ; C[C@H]1CCCN1Cc1nc2cc(NC(=O)c3ccc4c(cnn4CCNC(=O)CCCNc4cccc5c4C(=O)N(C4CCC(=O)NC4=O)C5=O)c3)ccc2[nH]1]

LQ076-154 was synthesized following the standard procedure for preparing LQ076-135 from intermediate 14 (13 mg, 0.02 mmol), 4-((2-(2,6-dioxopiperidin-3-yl)-1,3-dioxoisoindolin-4-yl)amino)butanoic acid (8.0 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-154 was obtained as yellow solid in TFA salt form (14.7 mg, 74%). ¹H NMR (800 MHz, Methanol-d₄) δ 8.41 (s, 1H), 8.26 (s, 1H), 8.20 (s, 1H), 8.01 (d, J=8.8 Hz, 1H), 7.67 (d, J=8.7 Hz, 2H), 7.54-7.49 (m, 2H), 6.96 (d, J=7.0 Hz, 1H), 6.93 (d, J=8.5 Hz, 1H), 4.96 (dd, J=13.7, 5.5 Hz, 1H), 4.82 (d, J=14.8 Hz, 1H), 4.63-4.56 (m, 3H), 3.78-3.69 (m, 4H), 3.51-3.45 (m, 1H), 3.08 (t, J=7.3 Hz, 2H), 2.78-2.71 (m, 1H), 2.68-2.57 (m, 2H), 2.44-2.38 (m, 1H), 2.19-2.07 (m, 4H), 2.02-1.98 (m, 1H), 1.87-1.81 (m, 1H), 1.74-1.68 (m, 2H), 1.53 (d, J=6.5 Hz, 3H). HRMS m/z [M+H]⁺ calcd for C₄₀H₄₃N₁₀O₆⁺ 759.3362, found 759.3334.

### Example 114

### Synthesis of LQ076-155

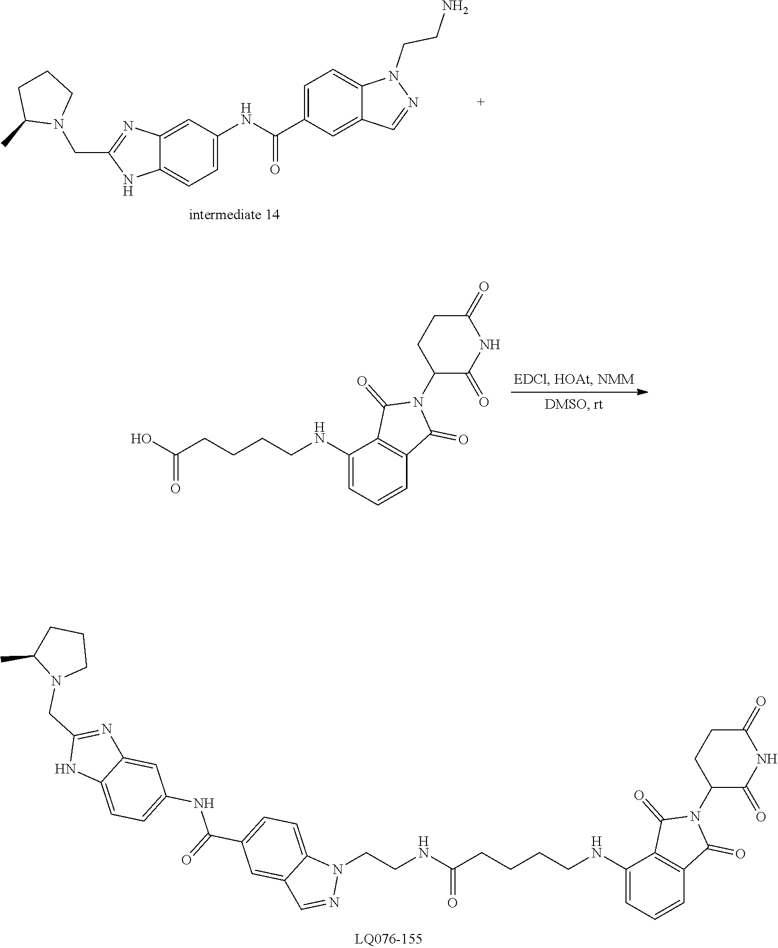 [SMILES: C[C@H]1CCCN1Cc1nc2cc(NC(=O)c3ccc4c(cnn4CCN)c3)ccc2[nH]1 ; O=C(O)CCCCNc1cccc2c1C(=O)N(C1CCC(=O)NC1=O)C2=O ; C[C@H]1CCCN1Cc1nc2cc(NC(=O)c3ccc4c(cnn4CCNC(=O)CCCCNc4cccc5c4C(=O)N(C4CCC(=O)NC4=O)C5=O)c3)ccc2[nH]1]

LQ076-155 was synthesized following the standard procedure for preparing LQ076-135 from intermediate 14 (13 mg, 0.02 mmol), 5-((2-(2,6-dioxopiperidin-3-yl)-1,3-dioxoisoindolin-4-yl)amino)pentanoic acid (8.4 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-155 was obtained as yellow solid in TFA salt form (13.9 mg, 69%). ¹H NMR (800 MHz, Methanol-d₄) δ 8.42 (s, 1H), 8.20 (d, J=11.8 Hz, 1H), 8.02 (d, J=8.8 Hz, 1H), 7.68-7.62 (m, 2H), 7.51 (d, J=8.7 Hz, 1H), 7.43 (t, J=7.8 Hz, 1H), 6.92 (d, J=8.5 Hz, 1H), 6.86 (d, J=7.0 Hz, 1H), 5.03 (dd, J=12.8, 5.6 Hz, 1H), 4.81 (d, J=14.6 Hz, 1H), 4.64-4.55 (m, 3H), 3.78-3.69 (m, 4H), 3.50-3.44 (m, 1H), 3.12 (t, J=7.1 Hz, 2H), 2.84-2.78 (m, 1H), 2.73-2.65 (m, 2H), 2.43-2.38 (m, 1H), 2.19-2.05 (m, 5H), 1.86-1.80 (m, 1H), 1.52 (d, J=6.5 Hz, 3H), 1.47-1.40 (m, 2H), 1.37-1.30 (m, 2H). HRMS m/z [M+H]⁺ calcd for C₄₁H₄₅N₁₀O₆⁺ 773.3518, found 773.3535.

### Example 115

### Synthesis of LQ076-156

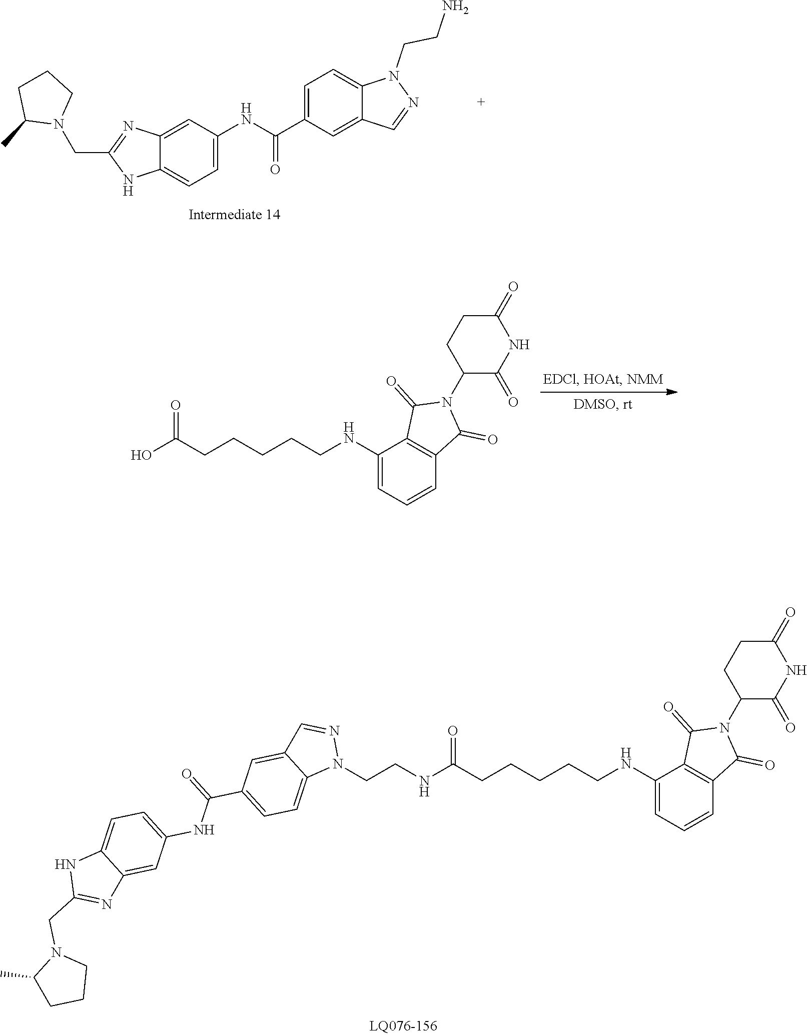 [SMILES: C[C@H]1CCCN1Cc1nc2cc(NC(=O)c3ccc4c(cnn4CCN)c3)ccc2[nH]1 ; O=C(O)CCCCCNc1cccc2c1C(=O)N(C1CCC(=O)NC1=O)C2=O ; C[C@H]1CCCN1Cc1nc2cc(NC(=O)c3ccc4c(cnn4CCNC(=O)CCCCCNc4cccc5c4C(=O)N(C4CCC(=O)NC4=O)C5=O)c3)ccc2[nH]1]

LQ076-156 was synthesized following the standard procedure for preparing LQ076-135 from intermediate 14 (13 mg, 0.02 mmol), 6-((2-(2,6-dioxopiperidin-3-yl)-1,3-dioxoisoindolin-4-yl)amino)hexanoic acid (8.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-156 was obtained as yellow solid in TFA salt form (15.7 mg, 77%). ¹H NMR (800 MHz, Methanol-d₄) δ 8.47 (s, 1H), 8.23 (d, J=4.3 Hz, 1H), 8.05 (d, J=8.7 Hz, 1H), 7.68 (d, J=8.9 Hz, 1H), 7.64 (d, J=8.7 Hz, 1H), 7.53 (d, J=8.7 Hz, 1H), 7.45 (t, J=7.8 Hz, 1H), 6.94 (d, J=8.5 Hz, 1H), 6.88 (d, J=7.1 Hz, 1H), 5.01 (dd, J=12.9, 5.5 Hz, 1H), 4.80 (d, J=14.6 Hz, 1H), 4.63-4.54 (m, 3H), 3.77-3.69 (m, 4H), 3.49-3.44 (m, 1H), 3.17 (t, J=7.1 Hz, 2H), 2.85-2.80 (m, 1H), 2.75-2.70 (m, 1H), 2.70-2.62 (m, 1H), 2.43-2.38 (m, 1H), 2.19-2.09 (m, 2H), 2.09-2.02 (m, 3H), 1.86-1.80 (m, 1H), 1.56-1.50 (m, 5H), 1.45-1.40 (m, 2H), 1.22-1.17 (m, 2H). HRMS m/z [M+H]⁺ calcd for C₄₂H₄₇N₁₀O₆⁺ 787.3675, found 787.3680.

### Example 116

### Synthesis of LQ076-157

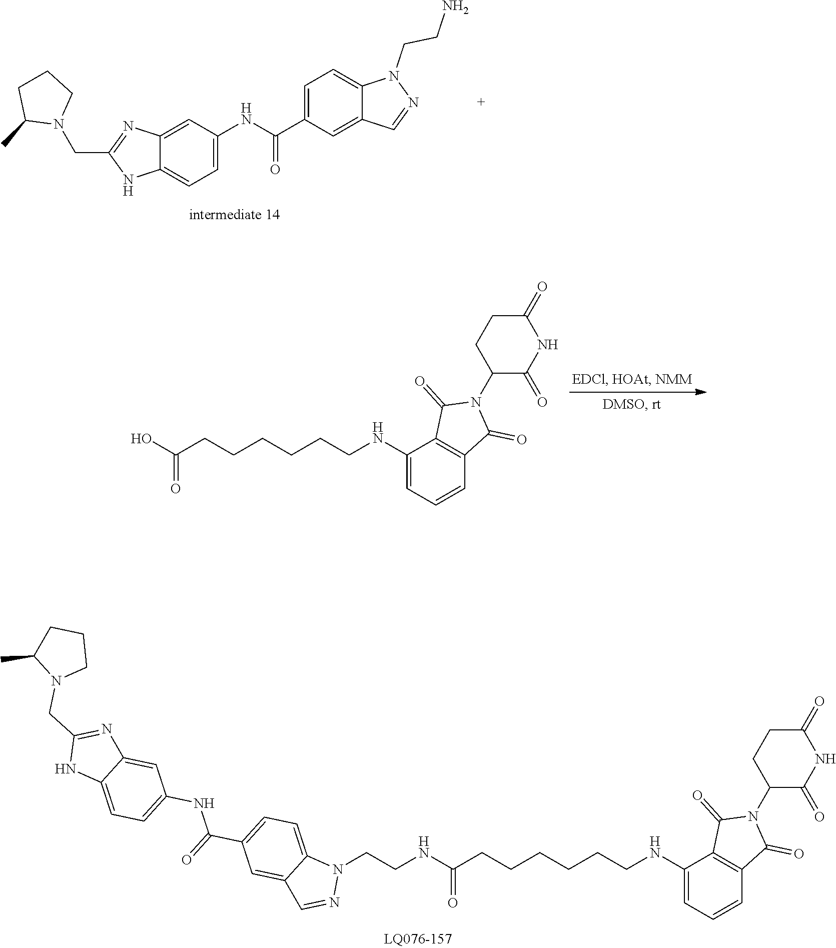 [SMILES: C[C@H]1CCCN1Cc1nc2cc(NC(=O)c3ccc4c(cnn4CCN)c3)ccc2[nH]1 ; O=C(O)CCCCCCNc1cccc2c1C(=O)N(C1CCC(=O)NC1=O)C2=O ; C[C@H]1CCCN1Cc1nc2cc(NC(=O)c3ccc4c(cnn4CCNC(=O)CCCCCCNc4cccc5c4C(=O)N(C4CCC(=O)NC4=O)C5=O)c3)ccc2[nH]1]

LQ076-157 was synthesized following the standard procedure for preparing LQ076-135 from intermediate 14 (13 mg, 0.02 mmol), 7-((2-(2,6-dioxopiperidin-3-yl)-1,3-dioxoisoindolin-4-yl)amino)heptanoic acid (9.1 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-157 was obtained as yellow solid in TFA salt form (14.4 mg, 70%). ¹H NMR (800 MHz, Methanol-d₄) δ 8.48 (s, 1H), 8.25 (s, 1H), 8.23 (s, 1H), 8.05 (d, J=8.8 Hz, 1H), 7.66 (d, J=8.8 Hz, 1H), 7.62 (d, J=8.7 Hz, 1H), 7.55 (d, J=8.7 Hz, 1H), 7.41 (t, J=7.8 Hz, 1H), 6.90-6.84 (m, 2H), 5.05 (dd, J=12.8, 5.6 Hz, 1H), 4.80 (d, J=14.7 Hz, 1H), 4.63-4.54 (m, 3H), 3.77-3.69 (m, 4H), 3.48-3.43 (m, 1H), 3.18 (t, J=7.1 Hz, 2H), 2.88-2.82 (m, 1H), 2.77-2.66 (m, 2H), 2.43-2.37 (m, 1H), 2.18-2.07 (m, 3H), 2.01 (t, J=7.5 Hz, 2H), 1.86-1.80 (m, 1H), 1.53-1.47 (m, 5H), 1.35-1.24 (m, 4H), 1.15-1.10 (m, 2H). HRMS m/z [M+H]⁺ calcd for C₄₃H₄₉N₁₀O₆⁺ 801.3831, found 801.3786.

### Example 117

### Synthesis of LQ076-158

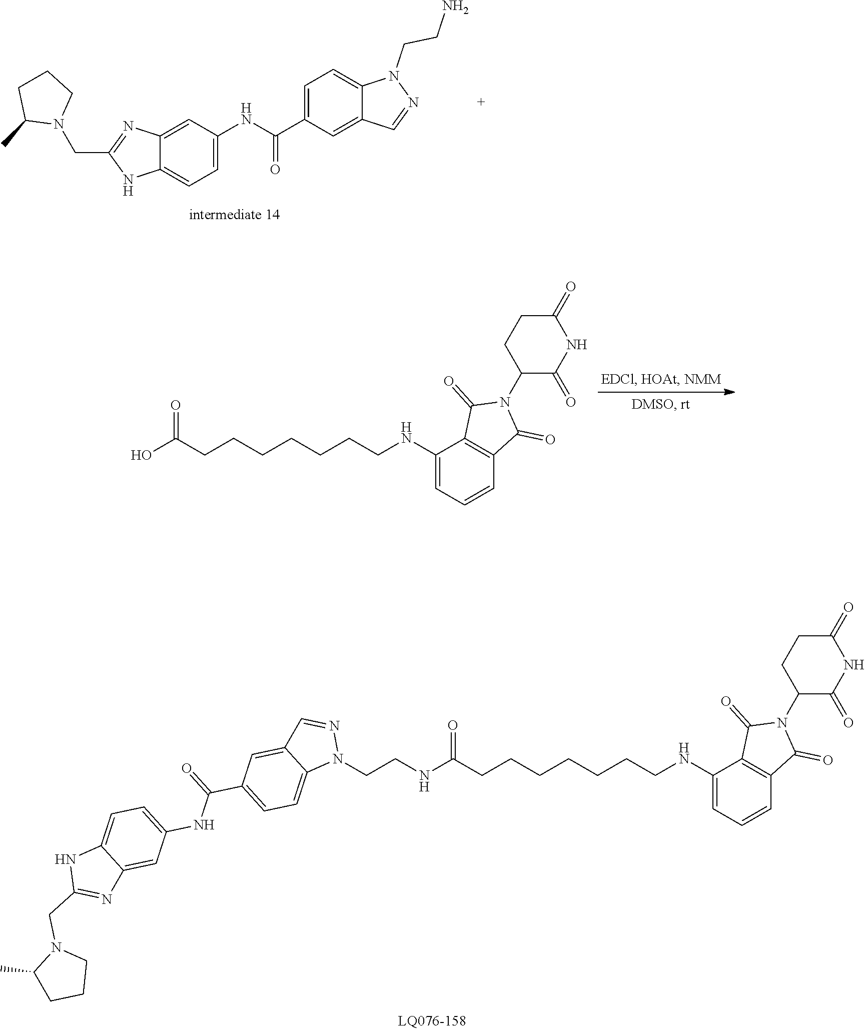 [SMILES: C[C@H]1CCCN1Cc1nc2cc(NC(=O)c3ccc4c(cnn4CCN)c3)ccc2[nH]1 ; O=C(O)CCCCCCCNc1cccc2c1C(=O)N(C1CCC(=O)NC1=O)C2=O ; C[C@H]1CCCN1Cc1nc2cc(NC(=O)c3ccc4c(cnn4CCNC(=O)CCCCCCCNc4cccc5c4C(=O)N(C4CCC(=O)NC4=O)C5=O)c3)ccc2[nH]1]

LQ076-158 was synthesized following the standard procedure for preparing LQ076-135 from intermediate 14 (13 mg, 0.02 mmol), 8-((2-(2,6-dioxopiperidin-3-yl)-1,3-dioxoisoindolin-4-yl)amino)octanoic acid (9.7 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-158 was obtained as yellow solid in TFA salt form (15.2 mg, 730). ¹H NMR (800 MHz, Methanol-d₄) δ 8.49 (s, 1H), 8.26 (s, 1H), 8.23 (s, 1H), 8.06 (d, J=8.8 Hz, 1H), 7.66 (d, J=8.9 Hz, 1H), 7.62 (d, J=8.7 Hz, 1H), 7.55 (d, J=8.7 Hz, 1H), 7.42 (t, J=7.8 Hz, 1H), 6.91 (d, J=7.0 Hz, 1H), 6.88 (d, J=8.6 Hz, 1H), 5.07 (dd, J=12.7, 5.6 Hz, 1H), 4.78-4.75 (m, 1H), 4.61 (t, J=5.8 Hz, 2H), 4.53 (d, J=14.7 Hz, 1H), 3.76-3.68 (m, 4H), 3.47-3.42 (m, 1H), 3.20 (t, J=7.2 Hz, 2H), 2.89-2.82 (m, 1H), 2.78-2.68 (m, 2H), 2.42-2.36 (m, 1H), 2.16-2.07 (m, 3H), 2.01 (t, J=7.4 Hz, 2H), 1.85-1.79 (m, 1H), 1.58-1.54 (m, 2H), 1.50 (d, J=6.6 Hz, 3H), 1.36-1.19 (m, 6H), 1.10-1.05 (m, 2H). HRMS m/z [M+H]⁺ calcd for C₄₄H₅₁N₁₀O₆⁺ 815.3988, found 815.3991.

### Example 118

### Synthesis of LQ076-159

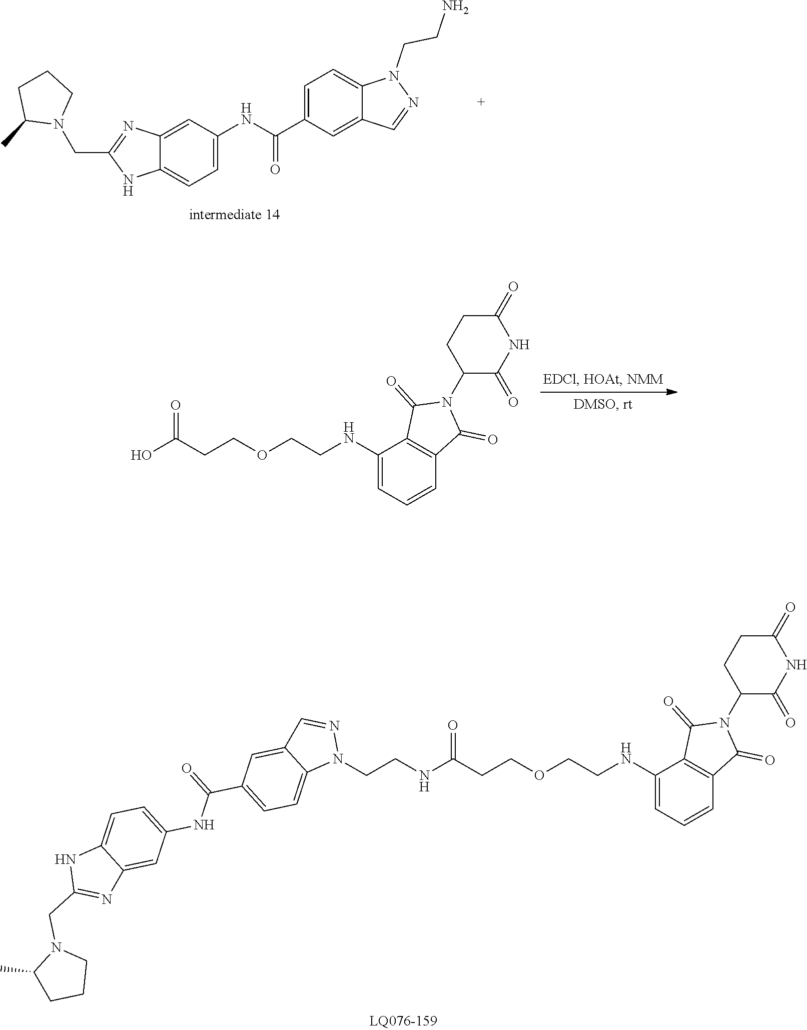 [SMILES: C[C@H]1CCCN1Cc1nc2cc(NC(=O)c3ccc4c(cnn4CCN)c3)ccc2[nH]1 ; O=C(O)CCOCCNc1cccc2c1C(=O)N(C1CCC(=O)NC1=O)C2=O ; C[C@H]1CCCN1Cc1nc2cc(NC(=O)c3ccc4c(cnn4CCNC(=O)CCOCCNc4cccc5c4C(=O)N(C4CCC(=O)NC4=O)C5=O)c3)ccc2[nH]1]

LQ076-159 was synthesized following the standard procedure for preparing LQ076-135 from intermediate 14 (13 mg, 0.02 mmol), 3-(2-((2-(2,6-dioxopiperidin-3-yl)-1,3-dioxoisoindolin-4-yl)amino)ethoxy)propanoic acid (7.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-159 was obtained as yellow solid in TFA salt form (12.4 mg, 61%). ¹H NMR (800 MHz, Methanol-d₄) δ 8.42 (s, 1H), 8.27 (s, 1H), 8.19 (s, 1H), 8.01 (d, J=8.8 Hz, 1H), 7.70-7.65 (m, 2H), 7.53 (d, J=8.6 Hz, 1H), 7.50 (t, J=7.8 Hz, 1H), 7.03 (d, J=8.5 Hz, 1H), 6.96 (d, J=7.0 Hz, 1H), 4.97 (dd, J=12.9, 5.6 Hz, 1H), 4.80 (d, J=14.7 Hz, 1H), 4.60 (t, J=6.1 Hz, 2H), 4.56 (d, J=14.7 Hz, 1H), 3.78-3.73 (m, 2H), 3.72-3.69 (m, 2H), 3.65-3.59 (m, 4H), 3.50-3.45 (m, 1H), 3.41 (t, J=5.2 Hz, 2H), 2.81-2.75 (m, 1H), 2.71-2.67 (m, 1H), 2.66-2.60 (m, 1H), 2.44-2.38 (m, 1H), 2.36 (t, J=6.0 Hz, 2H), 2.19-2.08 (m, 2H), 2.05-2.00 (m, 1H), 1.86-1.80 (m, 1H), 1.52 (d, J=6.5 Hz, 3H). HRMS m/z [M+H]⁺ calcd for C₄₁H₄₅N₁₀O₇⁺ 789.3467, found 789.3501.

### Example 119

### Synthesis of LQ076-160

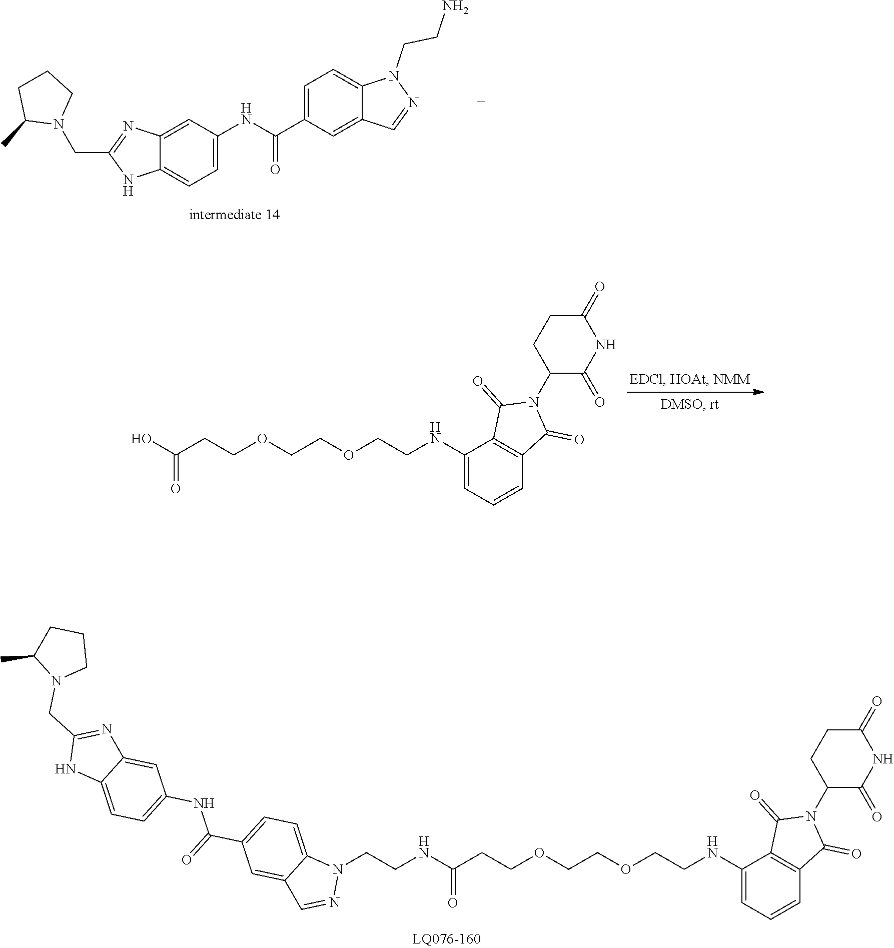 [SMILES: C[C@H]1CCCN1Cc1nc2cc(NC(=O)c3ccc4c(cnn4CCN)c3)ccc2[nH]1 ; O=C(O)CCOCCOCCNc1cccc2c1C(=O)N(C1CCC(=O)NC1=O)C2=O ; C[C@H]1CCCN1Cc1nc2cc(NC(=O)c3ccc4c(cnn4CCNC(=O)CCOCCOCCNc4cccc5c4C(=O)N(C4CCC(=O)NC4=O)C5=O)c3)ccc2[nH]1]

LQ076-160 was synthesized following the standard procedure for preparing LQ076-135 from intermediate 14 (13 mg, 0.02 mmol), 3-(2-(2-((2-(2,6-dioxopiperidin-3-yl)-1,3-dioxoisoindolin-4-yl)amino)ethoxy)ethoxy)propanoic acid (8.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-160 was obtained as yellow solid in TFA salt form (14.2 mg, 67%). ¹H NMR (800 MHz, Methanol-d₄) δ 8.43 (s, 1H), 8.27 (s, 1H), 8.19 (s, 1H), 8.01 (d, J=8.8 Hz, 1H), 7.68-7.63 (m, 2H), 7.56 (d, J=8.8 Hz, 1H), 7.45 (t, J=7.8 Hz, 1H), 6.99-6.93 (m, 2H), 5.03 (dd, J=12.7, 5.6 Hz, 1H), 4.81 (d, J=14.6 Hz, 1H), 4.61-4.53 (m, 3H), 3.77-3.71 (m, 2H), 3.70-3.63 (m, 4H), 3.62-3.56 (m, 4H), 3.55-3.51 (m, 2H), 3.49-3.44 (m, 1H), 3.42 (t, J=5.3 Hz, 2H), 2.92-2.78 (m, 1H), 2.77-2.65 (m, 2H), 2.43-2.37 (m, 1H), 2.33 (t, J=6.1 Hz, 2H), 2.19-2.07 (m, 3H), 1.86-1.79 (m, 1H), 1.51 (d, J=6.5 Hz, 3H). HRMS m/z [M+H]⁺ calcd for C₄₃H₄₉N₁₀O₈⁺ 833.3729, found 833.3760.

### Example 120

### Synthesis of LQ076-161

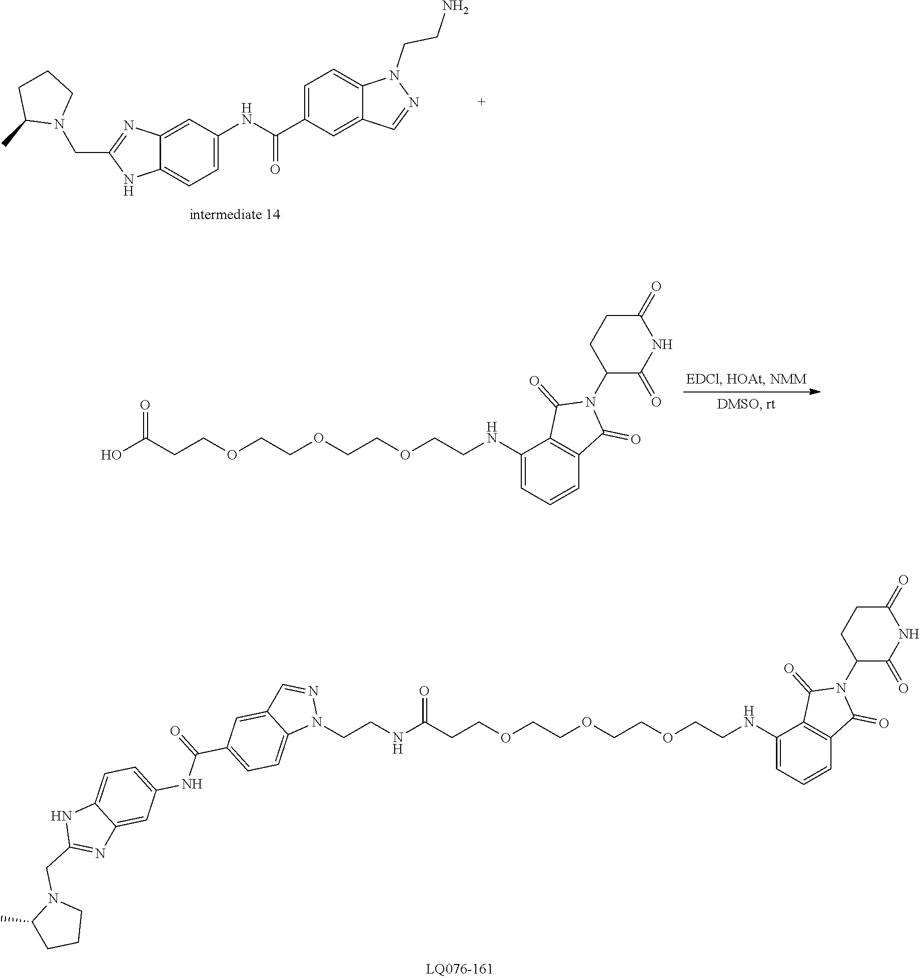 [SMILES: C[C@H]1CCCN1Cc1nc2cc(NC(=O)c3ccc4c(cnn4CCN)c3)ccc2[nH]1 ; O=C(O)CCOCCOCCOCCNc1cccc2c1C(=O)N(C1CCC(=O)NC1=O)C2=O ; C[C@H]1CCCN1Cc1nc2cc(NC(=O)c3ccc4c(cnn4CCNC(=O)CCOCCOCCOCCNc4cccc5c4C(=O)N(C4CCC(=O)NC4=O)C5=O)c3)ccc2[nH]1]

LQ076-161 was synthesized following the standard procedure for preparing LQ076-135 from intermediate 14 (13 mg, 0.02 mmol), 3-(2-(2-(2-((2-(2,6-dioxopiperidin-3-yl)-1,3-dioxoisoindolin-4-yl)amino)ethoxy)ethoxy)ethoxy)propanoic acid (9.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-161 was obtained as yellow solid in TFA salt form (15.3 mg, 72%). ¹H NMR (800 MHz, Methanol-d₄) δ 8.44 (s, 1H), 8.28 (s, 1H), 8.20 (s, 1H), 8.03 (d, J=8.7 Hz, 1H), 7.69-7.65 (m, 2H), 7.57 (d, J=8.7 Hz, 1H), 7.47 (t, J=7.8 Hz, 1H), 7.00-6.96 (m, 2H), 5.04 (dd, J=12.6, 5.6 Hz, 1H), 4.81 (d, J=14.6 Hz, 1H), 4.61-4.54 (m, 3H), 3.77-3.65 (m, 6H), 3.64-3.52 (m, 8H), 3.51-3.44 (m, 3H), 3.42 (t, J=5.3 Hz, 2H), 2.87-2.81 (m, 1H), 2.75-2.65 (m, 2H), 2.42-2.37 (m, 1H), 2.31 (t, J=6.1 Hz, 2H), 2.18-2.07 (m, 3H), 1.86-1.79 (m, 1H), 1.51 (d, J=6.5 Hz, 3H). HRMS m/z [M+H]⁺ calcd for C₄₅H₅₃N₁₀O₉⁺ 877.3991, found 877.4050.

### Example 121

### Synthesis of LQ076-162

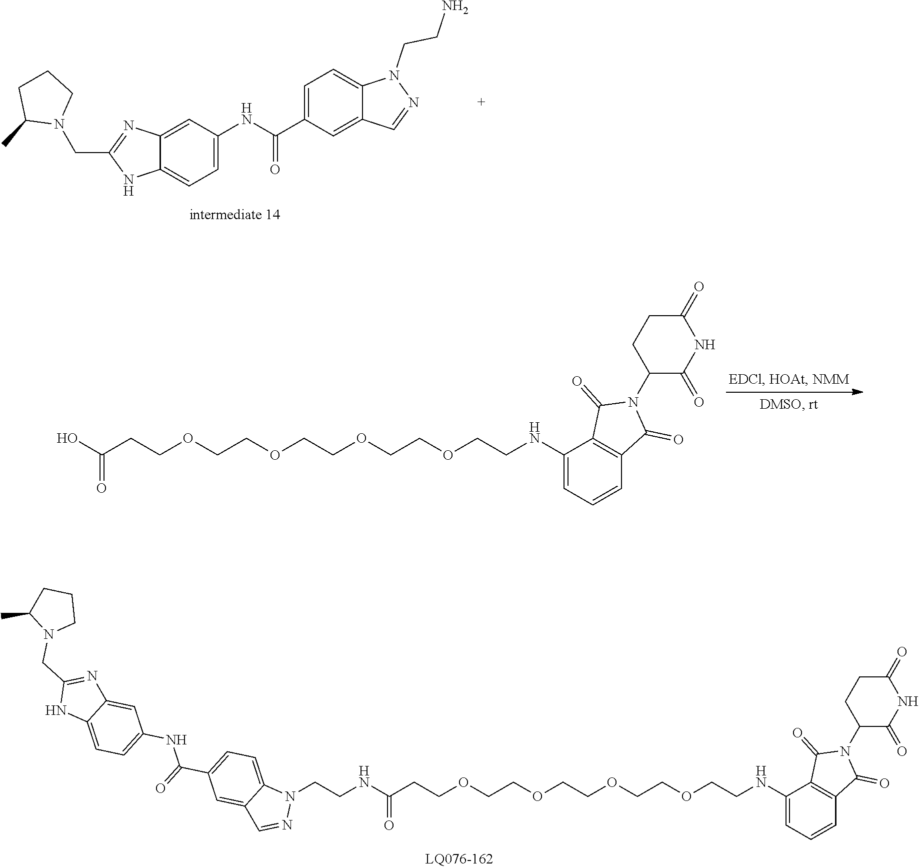 [SMILES: C[C@H]1CCCN1Cc1nc2cc(NC(=O)c3ccc4c(cnn4CCN)c3)ccc2[nH]1 ; O=C(O)CCOCCOCCOCCOCCNc1cccc2c1C(=O)N(C1CCC(=O)NC1=O)C2=O ; C[C@H]1CCCN1Cc1nc2cc(NC(=O)c3ccc4c(cnn4CCNC(=O)CCOCCOCCOCCOCCNc4cccc5c4C(=O)N(C4CCC(=O)NC4=O)C5=O)c3)ccc2[nH]1]

LQ076-162 was synthesized following the standard procedure for preparing LQ076-135 from intermediate 14 (13 mg, 0.02 mmol), 1-((2-(2,6-dioxopiperidin-3-yl)-1,3-dioxoisoindolin-4-yl)amino)-3,6,9,12-tetraoxapentadecan-15-oic acid (10.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-162 was obtained as yellow solid in TFA salt form (14.5 mg, 63%). ¹H NMR (800 MHz, Methanol-d₄) δ 8.46 (s, 1H), 8.30 (s, 1H), 8.21 (s, 1H), 8.04 (d, J=8.8 Hz, 1H), 7.70-7.66 (m, 2H), 7.57 (d, J=8.7 Hz, 1H), 7.49 (t, J=7.8 Hz, 1H), 7.03-6.98 (m, 2H), 5.04 (dd, J=12.7, 5.6 Hz, 1H), 4.81 (d, J=14.6 Hz, 1H), 4.62-4.55 (m, 3H), 3.77-3.72 (m, 2H), 3.70 (t, J=6.0 Hz, 2H), 3.67 (t, J=5.2 Hz, 2H), 3.64-3.55 (m, 10H), 3.54-3.51 (m, 2H), 3.50-3.45 (m, 3H), 3.43 (t, J=5.3 Hz, 2H), 2.88-2.82 (m, 1H), 2.76-2.67 (m, 2H), 2.43-2.38 (m, 1H), 2.31 (t, J=6.2 Hz, 2H), 2.18-2.08 (m, 3H), 1.86-1.80 (m, 1H), 1.51 (d, J=6.5 Hz, 3H). HRMS m/z [M+H]⁺ calcd for C₄₇H₅₇N₁₀O₁₀⁺ 921.4254, found 921.4290.

### Example 122

### Synthesis of LQ076-163

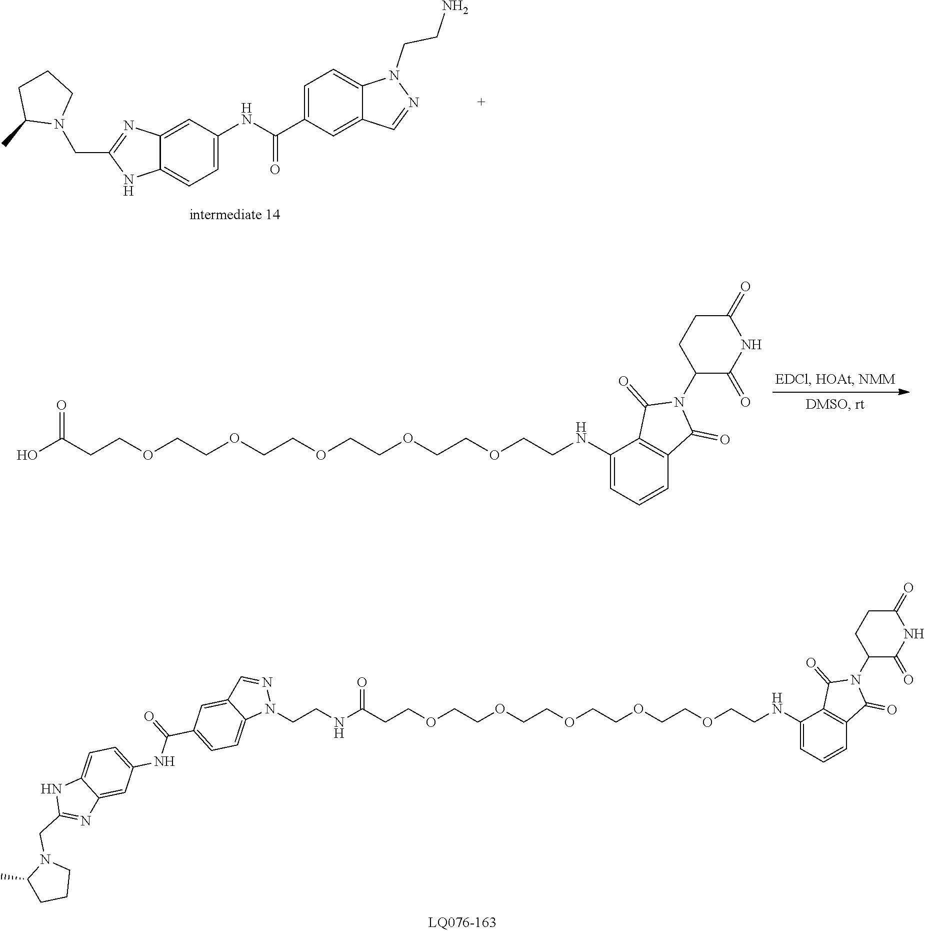 [SMILES: C[C@H]1CCCN1Cc1nc2cc(NC(=O)c3ccc4c(cnn4CCN)c3)ccc2[nH]1 ; O=C(O)CCOCCOCCOCCOCCOCCNc1cccc2c1C(=O)N(C1CCC(=O)NC1=O)C2=O ; C[C@H]1CCCN1Cc1nc2cc(NC(=O)c3ccc4c(cnn4CCNC(=O)CCOCCOCCOCCOCCOCCNc4cccc5c4C(=O)N(C4CCC(=O)NC4=O)C5=O)c3)ccc2[nH]1]

LQ076-163 was synthesized following the standard procedure for preparing LQ076-135 from intermediate 14 (13 mg, 0.02 mmol), 1-((2-(2,6-dioxopiperidin-3-yl)-1,3-dioxoisoindolin-4-yl)amino)-3,6,9,12,15-pentaoxaoctadecan-18-oic acid (11.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ076-163 was obtained as yellow solid in TFA salt form (16.6 mg, 69%). ¹H NMR (800 MHz, Methanol-d₄) δ 8.47 (s, 1H), 8.30 (s, 1H), 8.22 (s, 1H), 8.05 (d, J=8.8 Hz, 1H), 7.71-7.65 (m, 2H), 7.59 (d, J=8.6 Hz, 1H), 7.52-7.47 (m, 1H), 7.04-6.99 (m, 2H), 5.05 (dd, J=12.7, 5.5 Hz, 1H), 4.82 (d, J=14.7 Hz, 1H), 4.64-4.55 (m, 3H), 3.77-3.66 (m, 5H), 3.63-3.45 (m, 20H), 3.44 (t, J=5.3 Hz, 2H), 2.89-2.81 (m, 1H), 2.76-2.67 (m, 2H), 2.43-2.38 (m, 1H), 2.35-2.28 (m, 2H), 2.19-2.07 (m, 3H), 1.87-1.80 (m, 1H), 1.51 (d, J=6.3 Hz, 3H). HRMS m/z [M+H]⁺ calcd for C₄₉H₆₁N₁₀O₁₁⁺ 965.4516, found 965.4540.

### Example 123

### Synthesis of LQ081-100

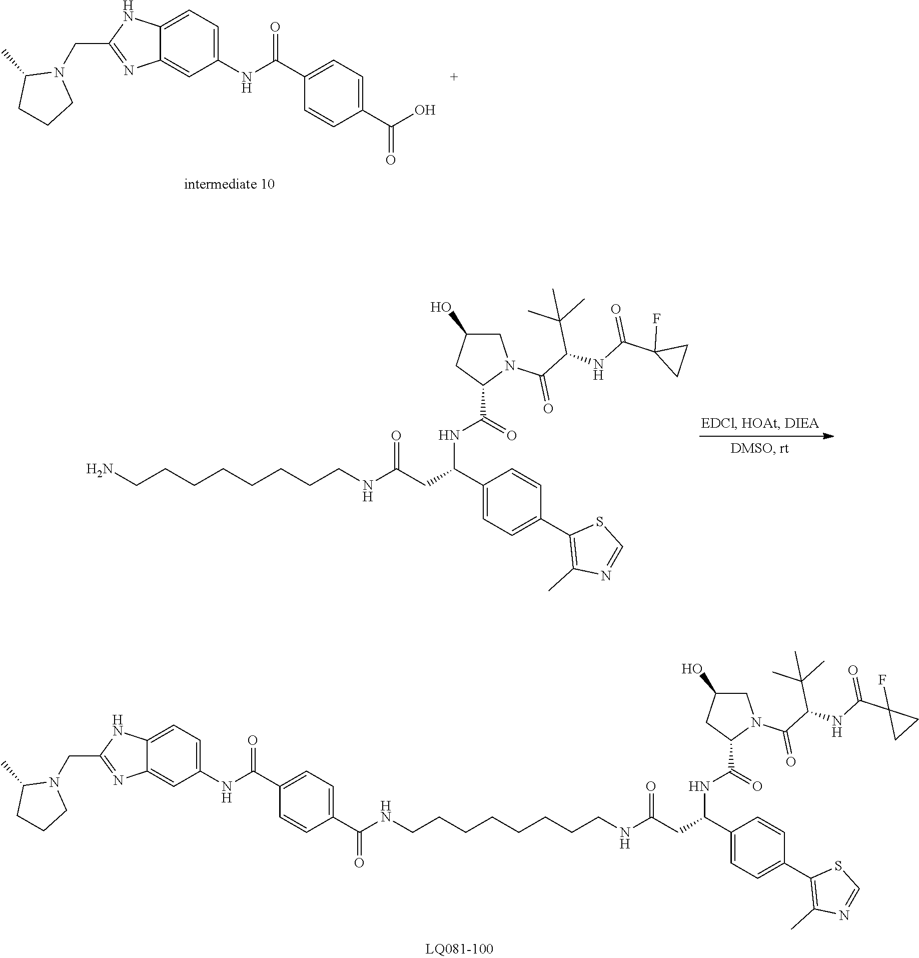 [SMILES: C[C@H]1CCCN1Cc1nc2cc(NC(=O)c3ccc(C(=O)O)cc3)ccc2[nH]1 ; Cc1ncsc1-c1ccc([C@H](CC(=O)NCCCCCCCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1]

LQ081-100 was synthesized following the standard procedure for preparing LQ076-105 from intermediate 10 (10 mg, 0.02 mmol), (2S,4R)—N—((S)-3-((8-aminooctyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (15.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ081-100 was obtained as white solid in TFA salt form (17.6 mg, 68%). ¹H NMR (600 MHz, Methanol-d₄) δ 8.97 (s, 1H), 8.30 (d, J=1.9 Hz, 1H), 8.06 (d, J=8.4 Hz, 2H), 7.97 (d, J=8.4 Hz, 2H), 7.68 (d, J=8.7 Hz, 1H), 7.57 (dd, J=8.7, 2.0 Hz, 1H), 7.52-7.45 (m, 4H), 5.33 (dd, J=8.5, 5.9 Hz, 1H), 4.82 (d, J=14.6 Hz, 1H), 4.75 (d, J=9.2 Hz, 1H), 4.63-4.55 (m, 2H), 4.49-4.44 (m, 1H), 3.87-3.82 (m, 1H), 3.80-3.70 (m, 3H), 3.51-3.43 (m, 1H), 3.38 (t, J=7.2 Hz, 2H), 3.17-3.09 (m, 1H), 3.09-3.02 (m, 1H), 2.86 (dd, J=14.0, 5.8 Hz, 1H), 2.78-2.72 (m, 1H), 2.50 (s, 3H), 2.44-2.35 (m, 1H), 2.26-2.19 (m, 1H), 2.18-2.06 (m, 2H), 2.01-1.94 (m, 1H), 1.88-1.78 (m, 1H), 1.59 (p, J=7.4 Hz, 2H), 1.51 (d, J=6.5 Hz, 3H), 1.42-1.23 (m, 12H), 1.21-1.15 (m, 2H), 1.07 (s, 9H). HRMS m/z [M+H]⁺ calcd for C₅₇H₇₄FN₁₀O₇S⁺ 1061.5441, found 1061.5461.

### Example 124

### Synthesis of LQ081-101

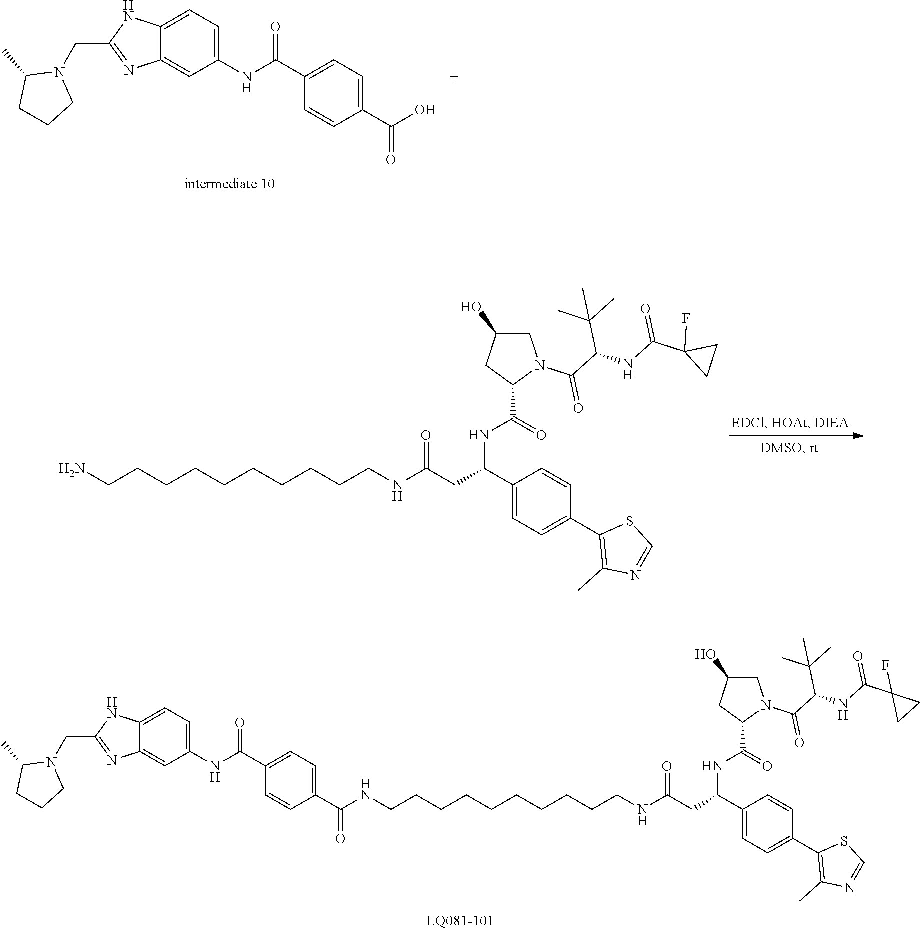 [SMILES: C[C@H]1CCCN1Cc1nc2cc(NC(=O)c3ccc(C(=O)O)cc3)ccc2[nH]1 ; Cc1ncsc1-c1ccc([C@H](CC(=O)NCCCCCCCCCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1]

LQ081-101 was synthesized following the standard procedure for preparing LQ076-105 from intermediate 10 (10 mg, 0.02 mmol), (2S,4R)—N—((S)-3-((10-aminodecyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (17 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ081-101 was obtained as white solid in TFA salt form (18.1 mg, 69%). ¹H NMR (600 MHz, Methanol-d₄) δ 8.82 (s, 1H), 8.17 (d, J=1.9 Hz, 1H), 7.94 (d, J=8.4 Hz, 2H), 7.86 (d, J=8.4 Hz, 2H), 7.56 (d, J=8.7 Hz, 1H), 7.43 (dd, J=8.7, 2.0 Hz, 1H), 7.40-7.32 (m, 4H), 5.21 (dd, J=8.5, 5.8 Hz, 1H), 4.67 (d, J=14.6 Hz, 1H), 4.64 (d, J=9.3 Hz, 1H), 4.50-4.46 (m, 1H), 4.44 (d, J=14.7 Hz, 1H), 4.36-4.33 (m, 1H), 3.76-3.70 (m, 1H), 3.69-3.59 (m, 3H), 3.38-3.33 (m, 1H), 3.30 (t, J=7.2 Hz, 2H), 3.05-2.97 (m, 1H), 2.96-2.89 (m, 1H), 2.74 (dd, J=14.0, 5.8 Hz, 1H), 2.64 (dd, J=14.0, 8.6 Hz, 1H), 2.38 (s, 3H), 2.33-2.25 (m, 1H), 2.13-2.07 (m, 1H), 2.06-1.94 (m, 1H), 1.89-1.82 (m, 1H), 1.76-1.67 (m, 1H), 1.52 (p, J=7.3 Hz, 2H), 1.39 (d, J=6.5 Hz, 3H), 1.32-1.08 (m, 14H), 1.07-1.00 (m, 2H), 0.96 (s, 9H). HRMS m/z [M+H]⁺ calcd for C₅₉H₇₈FN₁₀O₇S⁺ 1089.5754, found 1089.5825.

### Example 125

### Synthesis of LQ081-102

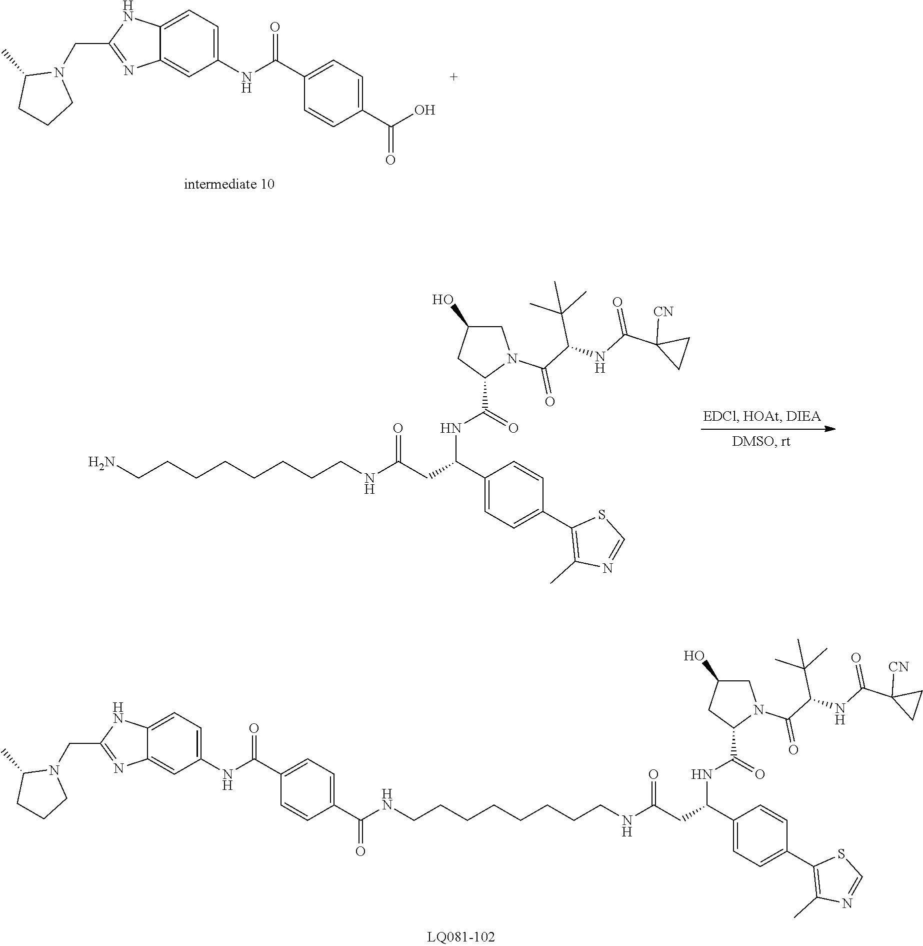 [SMILES: C[C@H]1CCCN1Cc1nc2cc(NC(=O)c3ccc(C(=O)O)cc3)ccc2[nH]1 ; Cc1ncsc1-c1ccc([C@H](CC(=O)NCCCCCCCCN)NC(=O)[C@@H]2C[C@@H](O)CN2C(=O)[C@@H](NC(=O)C2(C#N)CC2)C(C)(C)C)cc1 ; Cc1ncsc1-c1ccc([C@H](CC(=O)NCCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(C#N)CC2)C(C)(C)C)cc1]

LQ081-102 was synthesized following the standard procedure for preparing LQ076-105 from intermediate 10 (10 mg, 0.02 mmol), (2S,4R)—N—((S)-3-((8-aminooctyl)amino)-1-(4-(4-methylthiazol-5-yl)phenyl)-3-oxopropyl)-1-((S)-2-(1-cyanocyclopropane-1-carboxamido)-3,3-dimethylbutanoyl)-4-hydroxypyrrolidine-2-carboxamide (14.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ081-102 was obtained as white solid in TFA salt form (16.4 mg, 63%). ¹H NMR (600 MHz, Methanol-d₄) δ 8.98 (s, 1H), 8.30 (d, J=1.9 Hz, 1H), 8.06 (d, J=8.2 Hz, 2H), 7.97 (d, J=8.1 Hz, 2H), 7.68 (d, J=8.8 Hz, 1H), 7.56 (dd, J=8.8, 2.0 Hz, 1H), 7.49-7.42 (m, 4H), 5.33 (dd, J=8.5, 5.9 Hz, 1H), 4.81 (d, J=14.6 Hz, 1H), 4.69-4.65 (m, 1H), 4.63-4.54 (m, 2H), 4.47-4.44 (m, 1H), 3.81 (d, J=11.1 Hz, 1H), 3.78-3.70 (m, 3H), 3.51-3.43 (m, 1H), 3.38 (t, J=7.2 Hz, 2H), 3.18-3.10 (m, 1H), 3.09-3.01 (m, 1H), 2.86 (dd, J=14.1, 5.9 Hz, 1H), 2.80-2.72 (m, 1H), 2.50 (s, 3H), 2.44-2.36 (m, 1H), 2.23-2.08 (m, 3H), 2.00-1.93 (m, 1H), 1.87-1.78 (m, 1H), 1.68-1.55 (m, 6H), 1.51 (d, J=6.5 Hz, 3H), 1.40-1.23 (m, 8H), 1.21-1.13 (m, 2H), 1.07 (s, 9H). HRMS m/z [M+H]⁺ calcd for C₅₈H₇₄N₁₁O₇S⁺ 1068.5488, found 1068.5527.

### Example 126

### Synthesis of LQ081-103

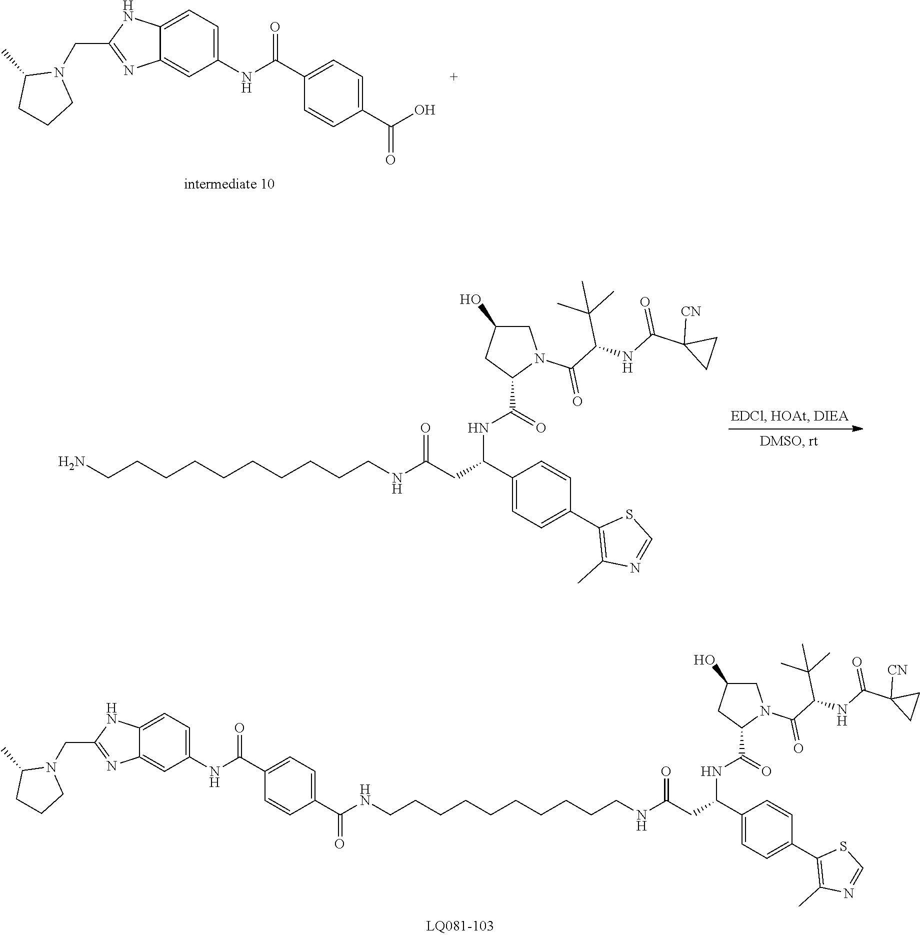 [SMILES: C[C@H]1CCCN1Cc1nc2cc(NC(=O)c3ccc(C(=O)O)cc3)ccc2[nH]1 ; Cc1ncsc1-c1ccc([C@H](CC(=O)NCCCCCCCCCCN)NC(=O)[C@@H]2C[C@@H](O)CN2C(=O)[C@@H](NC(=O)C2(C#N)CC2)C(C)(C)C)cc1 ; Cc1ncsc1-c1ccc([C@H](CC(=O)NCCCCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(C#N)CC2)C(C)(C)C)cc1]

LQ081-103 was synthesized following the standard procedure for preparing LQ076-105 from intermediate 10 (10 mg, 0.02 mmol), (2S,4R)—N—((S)-3-((10-aminodecyl)amino)-1-(4-(4-methylthiazol-5-yl)phenyl)-3-oxopropyl)-1-((S)-2-(1-cyanocyclopropane-1-carboxamido)-3,3-dimethylbutanoyl)-4-hydroxypyrrolidine-2-carboxamide (16.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ081-103 was obtained as white solid in TFA salt form (17.9 mg, 67%). ¹H NMR (600 MHz, Methanol-d₄) δ 8.97 (s, 1H), 8.30 (d, J=1.7 Hz, 1H), 8.06 (d, J=8.1 Hz, 2H), 7.97 (d, J=8.2 Hz, 2H), 7.68 (d, J=8.7 Hz, 1H), 7.55 (dd, J=8.8, 2.0 Hz, 1H), 7.48-7.44 (m, 3H), 7.44-7.36 (m, 1H), 5.32 (dd, J=8.6, 5.7 Hz, 1H), 4.79 (d, J=14.5 Hz, 1H), 4.67 (d, J=8.8 Hz, 1H), 4.62-4.49 (m, 2H), 4.47-4.44 (m, 1H), 3.81 (d, J=11.2 Hz, 1H), 3.78-3.72 (m, 3H), 3.50-3.44 (m, 1H), 3.41 (t, J=7.1 Hz, 2H), 3.17-3.10 (m, 1H), 3.08-3.00 (m, 1H), 2.86 (dd, J=14.0, 5.9 Hz, 1H), 2.76 (dd, J=14.0, 8.6 Hz, 1H), 2.50 (s, 3H), 2.44-2.36 (m, 1H), 2.24-2.07 (m, 2H), 2.00-1.93 (m, 1H), 1.87-1.79 (m, 1H), 1.68-1.55 (m, 9H), 1.51 (d, J=6.5 Hz, 3H), 1.42-1.20 (m, 10H), 1.18-1.11 (m, 2H), 1.07 (s, 9H). HRMS m/z [M+H]⁺ calcd for C₆₀H₇₈N₁₁O₇S⁺ 1096.5801, found 1096.5721.

### Example 127

### Synthesis of LQ081-104

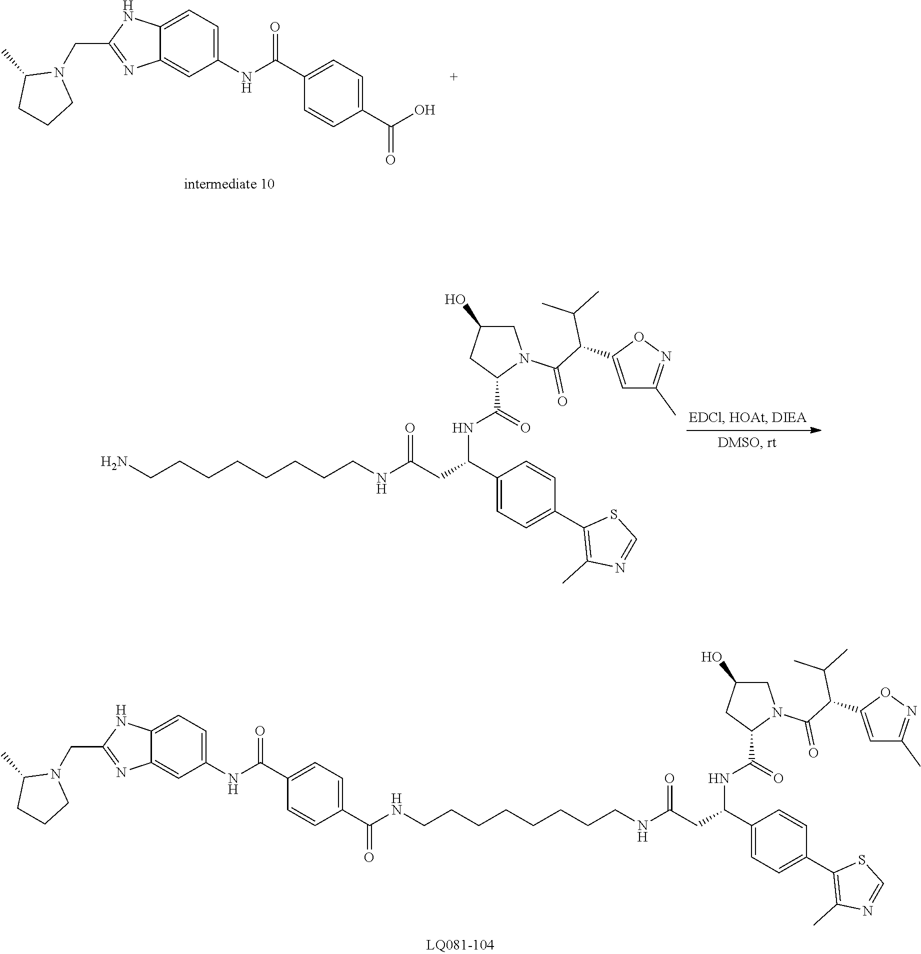

LQ081-104 was synthesized following the standard procedure for preparing LQ076-105 from intermediate 10 (10 mg, 0.02 mmol), (2S,4R)—N—((S)-3-((8-aminooctyl)amino)-1-(4-(4-methylthiazol-5-yl)phenyl)-3-oxopropyl)-4-hydroxy-1-((R)-3-methyl-2-(3-methylisoxazol-5-yl)butanoyl)pyrrolidine-2-carboxamide (13.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ081-104 was obtained as white solid in TFA salt form (14.4 mg, 58%). ¹H NMR (600 MHz, Methanol-d₄) δ 9.05 (s, 1H), 8.33 (s, 1H), 8.05 (d, J=8.3 Hz, 2H), 7.97 (d, J=8.1 Hz, 2H), 7.70 (d, J=8.7 Hz, 1H), 7.59 (dd, J=8.8, 2.0 Hz, 1H), 7.51-7.38 (m, 4H), 6.28-6.19 (m, 1H), 5.38-5.26 (m, 1H), 4.84 (d, J=14.5 Hz, 1H), 4.63-4.55 (m, 1H), 4.52-4.43 (m, 2H), 3.93-3.86 (m, 1H), 3.82-3.65 (m, 3H), 3.61 (d, J=10.6 Hz, 1H), 3.51-3.43 (m, 1H), 3.41-3.35 (m, 2H), 3.15-3.02 (m, 2H), 2.89-2.70 (m, 2H), 2.51 (s, 3H), 2.46-2.36 (m, 2H), 2.28-2.07 (m, 5H), 2.01-1.94 (m, 1H), 1.87-1.79 (m, 1H), 1.64-1.56 (m, 2H), 1.52 (d, J=6.5 Hz, 3H), 1.39-1.14 (m, 9H), 1.09-1.05 (m, 3H), 0.88 (dd, J=18.8, 6.7 Hz, 3H). HRMS m/z [M+H]⁺ calcd for C₅₆H₇₁N₁₀O₇S⁺ 1027.5222, found 1027.5257.

### Example 128

### Synthesis of LQ081-105

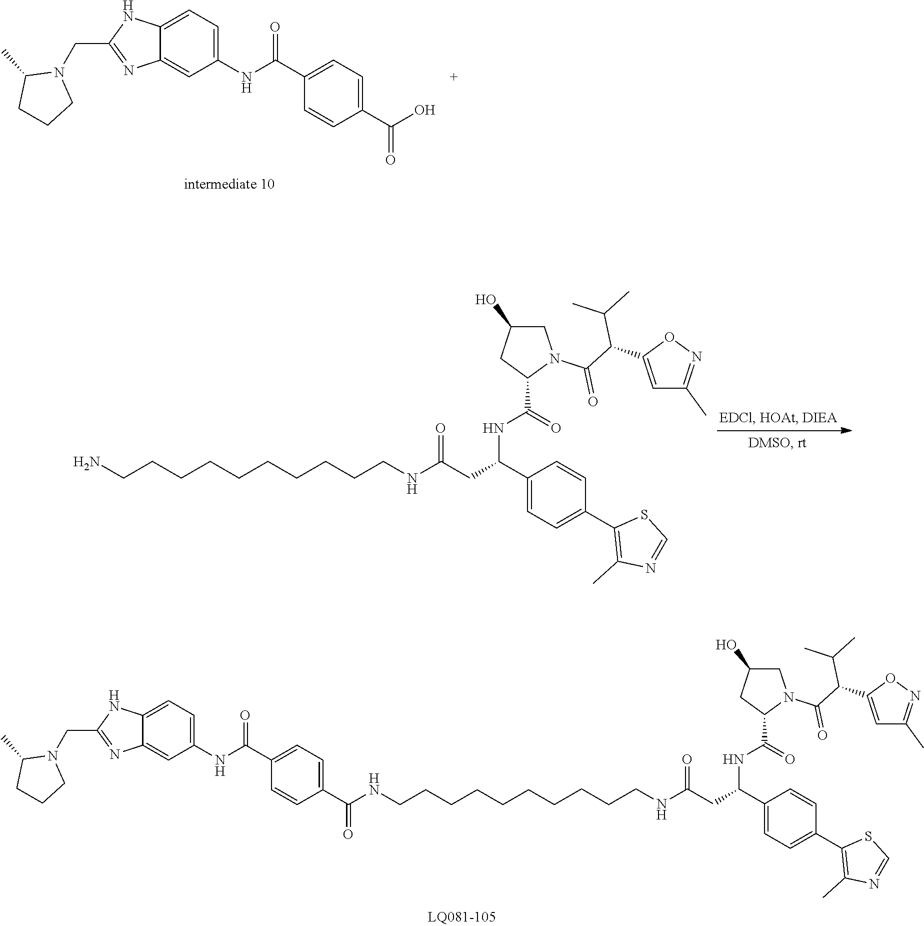

LQ081-105 was synthesized following the standard procedure for preparing LQ076-105 from intermediate 10 (10 mg, 0.02 mmol), (2S,4R)—N—((S)-3-((10-aminodecyl)amino)-1-(4-(4-methylthiazol-5-yl)phenyl)-3-oxopropyl)-4-hydroxy-1-((R)-3-methyl-2-(3-methylisoxazol-5-yl)butanoyl)pyrrolidine-2-carboxamide (15.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ081-105 was obtained as white solid in TFA salt form (16.3 mg, 64%). ¹H NMR (600 MHz, Methanol-d₄) δ 8.99 (s, 1H), 8.31 (d, J=1.9 Hz, 1H), 8.05 (d, J=8.0 Hz, 2H), 7.97 (d, J=8.2 Hz, 2H), 7.68 (d, J=8.8 Hz, 1H), 7.58 (dd, J=8.7, 2.0 Hz, 1H), 7.50-7.37 (m, 5H), 6.28-6.22 (m, 1H), 5.36-5.27 (m, 1H), 4.82 (d, J=14.7 Hz, 1H), 4.61-4.43 (m, 3H), 3.93-3.86 (m, 1H), 3.82-3.59 (m, 5H), 3.50-3.38 (m, 3H), 3.14-2.99 (m, 2H), 2.89-2.82 (m, 1H), 2.80-2.70 (m, 1H), 2.50 (s, 3H), 2.47-2.35 (m, 2H), 2.28-2.22 (m, 3H), 2.19-2.07 (m, 2H), 2.01-1.94 (m, 1H), 1.87-1.78 (m, 1H), 1.63 (q, J=7.3 Hz, 2H), 1.51 (d, J=6.5 Hz, 3H), 1.41-1.10 (m, 9H), 1.07 (dd, J=6.6, 2.6 Hz, 3H), 0.88 (dd, J=18.9, 6.7 Hz, 3H). HRMS m/z [M+H]⁺ calcd for C₅₈H₇₅N₁₀O₇S⁺ 1055.5535, found 1055.5540.

### Synthesis of LQ081-106

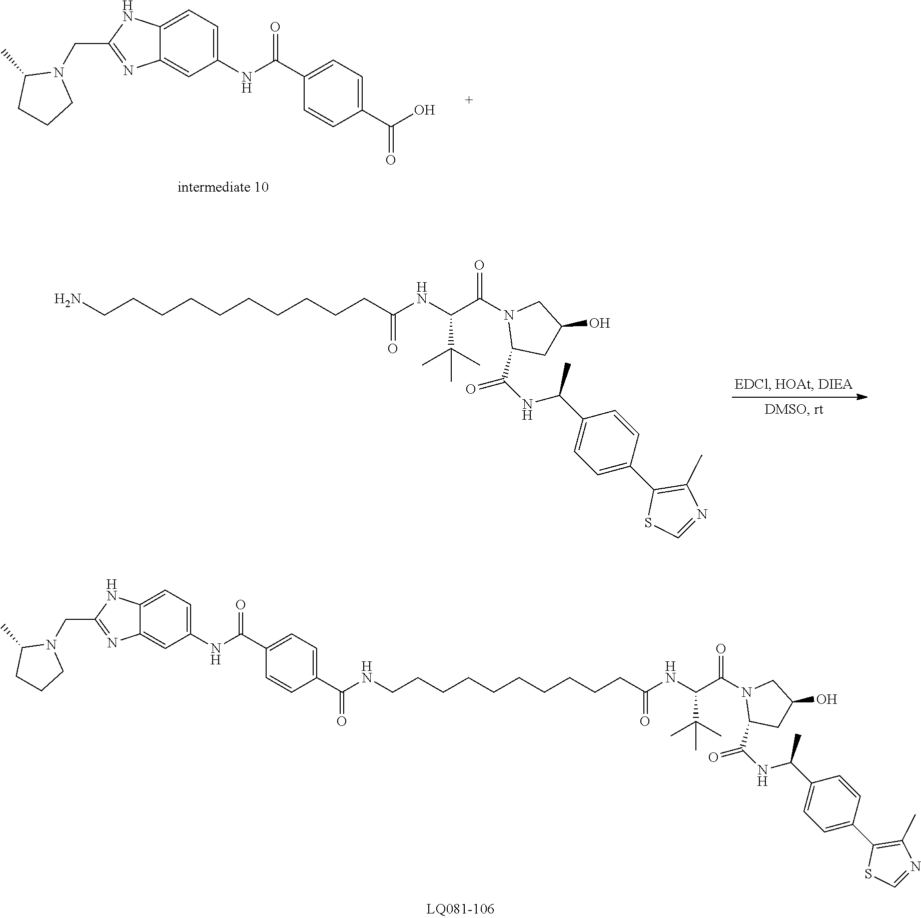

LQ081-106 was synthesized following the standard procedure for preparing LQ076-105 from intermediate 10 (10 mg, 0.02 mmol), (2R,4S)-1-((S)-2-(11-aminoundecanamido)-3,3-dimethylbutanoyl)-4-hydroxy-N—((S)-1-(4-(4-methylthiazol-5-yl)phenyl)ethyl)pyrrolidine-2-carboxamide (13.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ081-106 was obtained as white solid in TFA salt form (18.4 mg, 75%). ¹H NMR (600 MHz, Methanol-d₄) δ 8.98 (s, 1H), 8.30 (d, J=2.0 Hz, 1H), 8.05 (d, J=8.3 Hz, 2H), 7.96 (d, J=8.2 Hz, 2H), 7.68 (d, J=8.8 Hz, 1H), 7.57 (dd, J=8.7, 2.0 Hz, 1H), 7.54-7.51 (m, 2H), 7.47-7.44 (m, 2H), 5.06-5.00 (m, 1H), 4.81 (d, J=14.6 Hz, 1H), 4.60-4.54 (m, 2H), 4.51-4.49 (m, 1H), 4.49-4.44 (m, 1H), 3.96 (dd, J=10.8, 5.0 Hz, 1H), 3.78-3.68 (m, 3H), 3.50-3.38 (m, 3H), 2.51 (s, 3H), 2.44-2.35 (m, 1H), 2.34-2.26 (m, 1H), 2.24-2.07 (m, 4H), 1.86-1.78 (m, 1H), 1.57 (dd, J=70.8, 7.0 Hz, 8H), 1.46 (d, J=7.0 Hz, 3H), 1.42-1.25 (m, 12H), 1.07 (s, 9H). HRMS m/z [M+H]⁺ calcd for C₅₅H₇₄N₉O₆S⁺ 988.5477, found 988.5487.

### Synthesis of LQ081-107

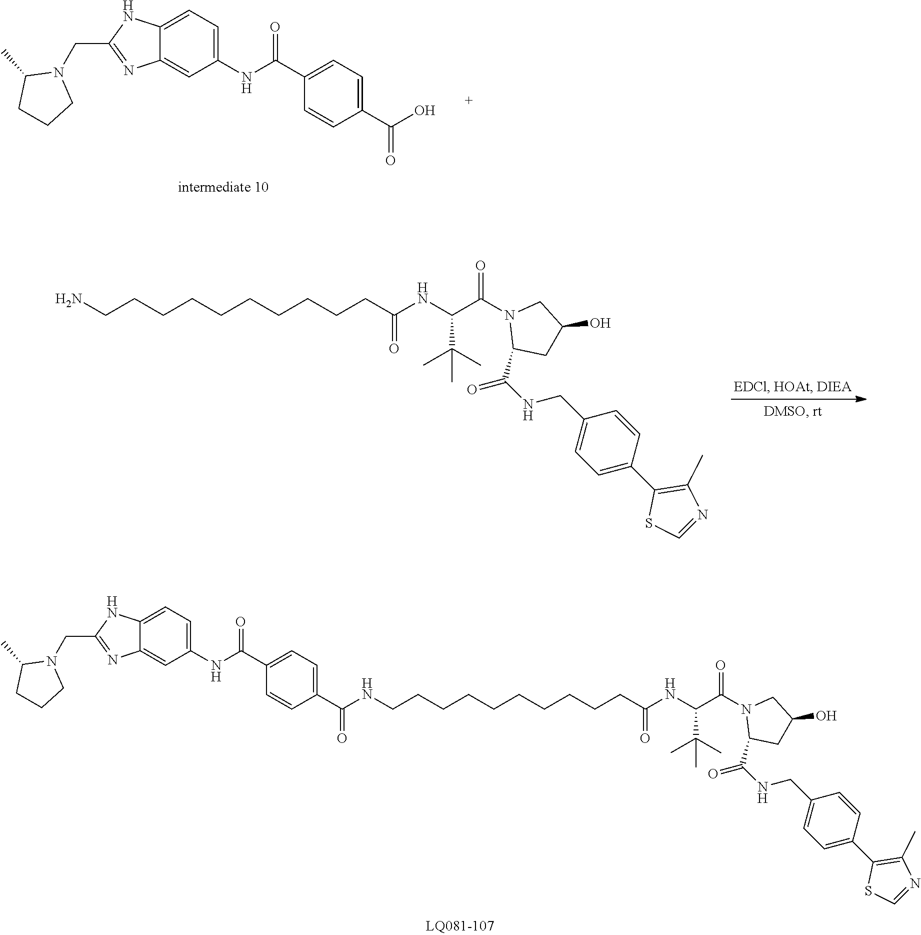

LQ081-107 was synthesized following the standard procedure for preparing LQ076-105 from intermediate 10 (10 mg, 0.02 mmol), (2R,4S)-1-((S)-2-(11-aminoundecanamido)-3,3-dimethylbutanoyl)-4-hydroxy-N-(4-(4-methylthiazol-5-yl)benzyl)pyrrolidine-2-carboxamide (12.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ081-107 was obtained as white solid in TFA salt form (17.7 mg, 73%). ¹H NMR (600 MHz, Methanol-d₄) δ 9.04 (s, 1H), 8.34 (d, J=2.0 Hz, 1H), 8.05 (d, J=8.3 Hz, 2H), 7.97 (d, J=8.3 Hz, 2H), 7.69 (d, J=8.7 Hz, 1H), 7.58 (dd, J=8.7, 2.0 Hz, 1H), 7.47-7.43 (m, 2H), 7.42-7.38 (m, 2H), 4.84 (d, J=14.6 Hz, 1H), 4.62-4.57 (m, 2H), 4.56-4.49 (m, 2H), 4.47-4.44 (m, 1H), 4.35 (d, J=15.6 Hz, 1H), 4.02 (dd, J=10.9, 4.9 Hz, 1H), 3.78-3.70 (m, 3H), 3.50-3.43 (m, 1H), 3.40 (t, J=7.1 Hz, 2H), 2.51 (s, 3H), 2.43-2.37 (m, 1H), 2.31-2.26 (m, 1H), 2.23-2.07 (m, 3H), 2.05-1.99 (m, 1H), 1.86-1.79 (m, 1H), 1.63 (p, J=7.2 Hz, 2H), 1.51 (d, J=6.5 Hz, 3H), 1.48-1.42 (m, 1H), 1.41-1.17 (m, 14H), 1.09 (s, 9H). HRMS m/z [M+H]⁺ calcd for C₅₄H₇₂N₉O₆S⁺ 974.5321, found 974.5351.

### Example 131

### Synthesis of LQ081-108

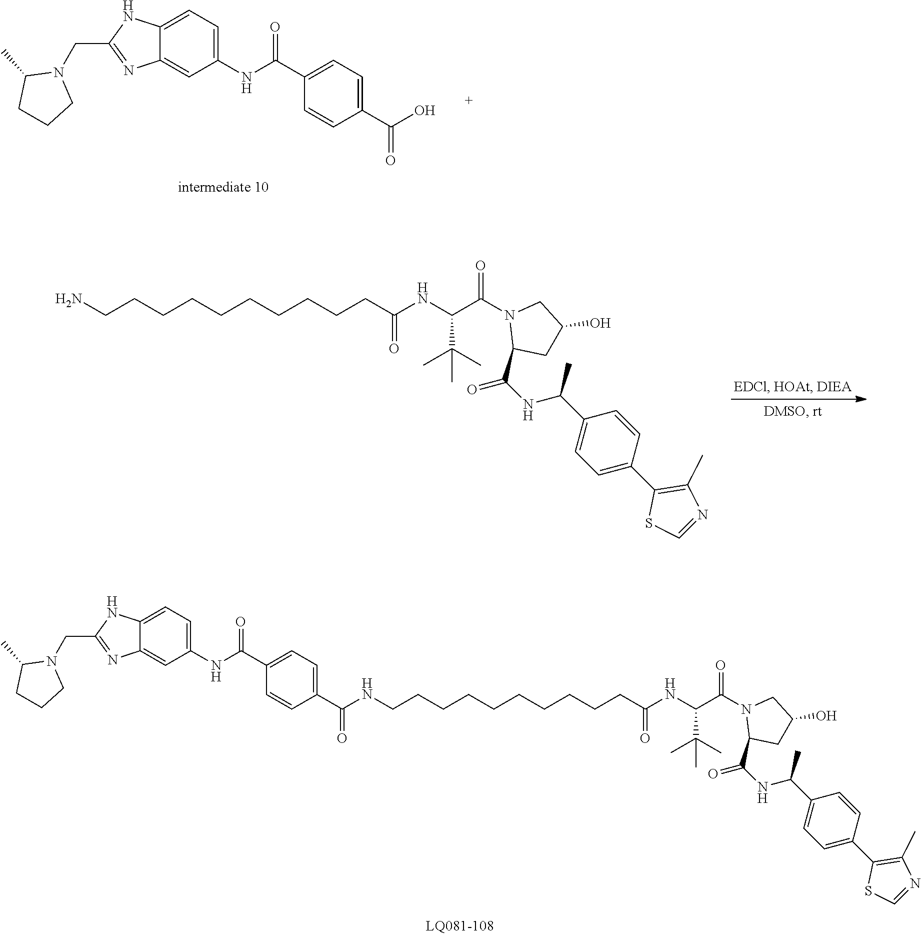

LQ081-108 was synthesized following the standard procedure for preparing LQ076-105 from intermediate 10 (10 mg, 0.02 mmol), (2S,4R)-1-((S)-2-(11-aminoundecanamido)-3,3-dimethylbutanoyl)-4-hydroxy-N—((S)-1-(4-(4-methylthiazol-5-yl)phenyl)ethyl)pyrrolidine-2-carboxamide (13.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ081-108 was obtained as white solid in TFA salt form (18.5 mg, 76%). ¹H NMR (600 MHz, Methanol-d₄) δ 9.03 (s, 1H), 8.31 (d, J=2.0 Hz, 1H), 8.08-8.03 (m, 2H), 7.99-7.94 (m, 2H), 7.68 (d, J=8.8 Hz, 1H), 7.57 (dd, J=8.7, 2.0 Hz, 1H), 7.48-7.42 (m, 4H), 5.01 (q, J=6.9 Hz, 1H), 4.82 (d, J=14.5 Hz, 1H), 4.65-4.62 (m, 1H), 4.62-4.55 (m, 2H), 4.46-4.43 (m, 1H), 3.89 (d, J=11.1 Hz, 1H), 3.79-3.70 (m, 3H), 3.50-3.37 (m, 3H), 2.50 (s, 3H), 2.43-2.36 (m, 1H), 2.34-2.07 (m, 4H), 2.00-1.93 (m, 1H), 1.86-1.79 (m, 1H), 1.69-1.57 (m, 5H), 1.54-1.49 (m, 6H), 1.45-1.30 (m, 12H), 1.05 (s, 9H). HRMS m/z [M+H]⁺ calcd for C₅₅H₇₄N₉O₆S⁺ 988.5477, found 988.5487.

### Example 132

### Synthesis of LQ081-109

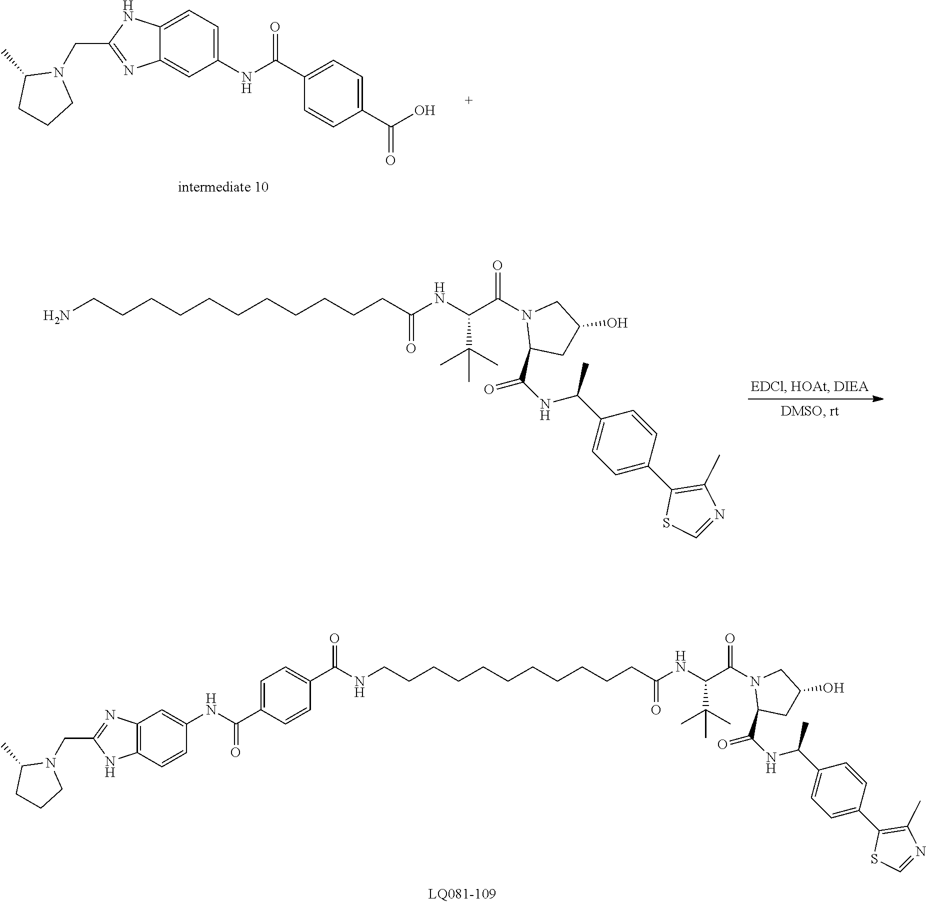

LQ081-109 was synthesized following the standard procedure for preparing LQ076-105 from intermediate 10 (10 mg, 0.02 mmol), (2S,4R)-1-((S)-2-(12-aminododecanamido)-3,3-dimethylbutanoyl)-4-hydroxy-N—((S)-1-(4-(4-methylthiazol-5-yl)phenyl)ethyl)pyrrolidine-2-carboxamide (13.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ081-109 was obtained as white solid in TFA salt form (17.3 mg, 70%). ¹H NMR (600 MHz, Methanol-d₄) δ 9.06 (s, 1H), 8.32 (d, J=2.0 Hz, 1H), 8.08-8.03 (m, 2H), 7.99-7.94 (m, 2H), 7.69 (d, J=8.7 Hz, 1H), 7.58 (dd, J=8.8, 2.0 Hz, 1H), 7.49-7.42 (m, 4H), 5.01 (q, J=6.9 Hz, 1H), 4.83 (d, J=14.6 Hz, 1H), 4.65-4.56 (m, 3H), 4.46-4.43 (m, 1H), 3.90 (d, J=11.0 Hz, 1H), 3.79-3.70 (m, 3H), 3.49-3.39 (m, 3H), 2.50 (s, 3H), 2.43-2.37 (m, 1H), 2.35-2.19 (m, 2H), 2.18-2.08 (m, 1H), 1.99-1.94 (m, 1H), 1.86-1.79 (m, 1H), 1.69-1.57 (m, 5H), 1.54-1.49 (m, 6H), 1.45-1.30 (m, 14H), 1.06 (s, 9H). HRMS m/z [M+H]⁺ calcd for C₅₆H₇₆N₉O₆S⁺ 1002.5634, found 1002.5669.

### Example 133

### Synthesis of LQ081-122

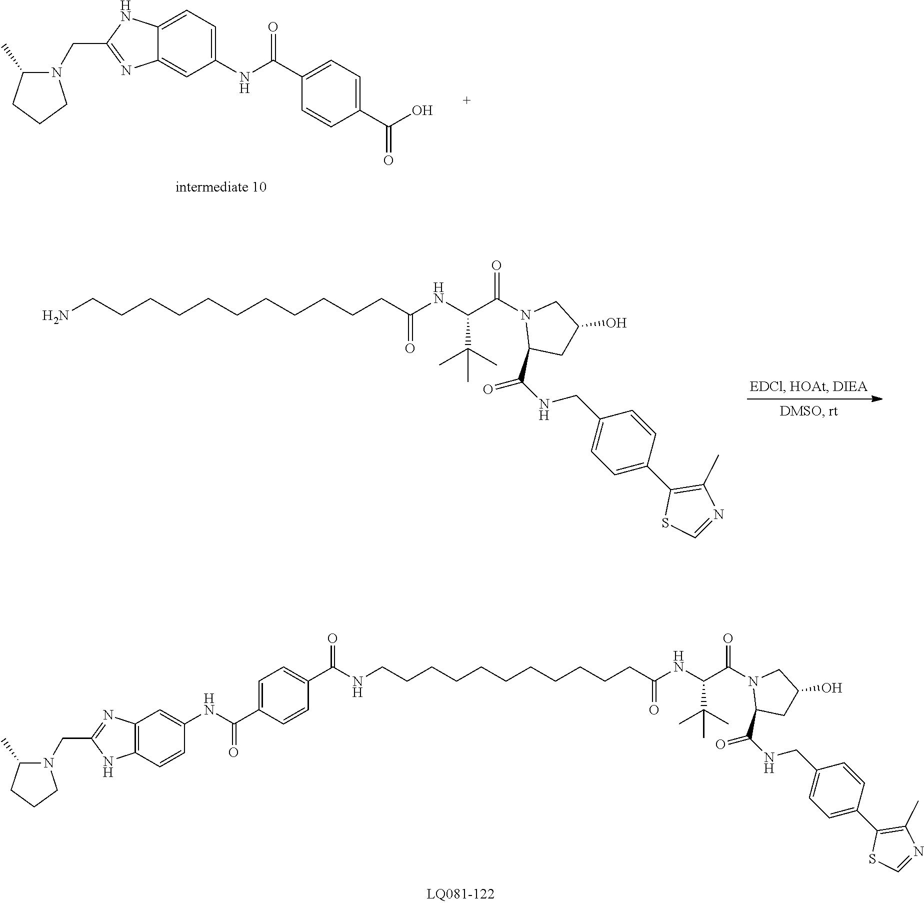

LQ081-122 was synthesized following the standard procedure for preparing LQ076-105 from intermediate 10 (10 mg, 0.02 mmol), (2S,4R)-1-((S)-2-(12-aminododecanamido)-3,3-dimethylbutanoyl)-4-hydroxy-N-(4-(4-methylthiazol-5-yl)benzyl)pyrrolidine-2-carboxamide (13.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ081-122 was obtained as white solid in TFA salt form (15.9 mg, 65%). ¹H NMR (600 MHz, Methanol-d₄) δ 9.00 (s, 1H), 8.31 (d, J=1.9 Hz, 1H), 8.07-8.03 (m, 2H), 7.99-7.94 (m, 2H), 7.68 (d, J=8.8 Hz, 1H), 7.57 (dd, J=8.7, 2.0 Hz, 1H), 7.50-7.41 (m, 4H), 4.81 (d, J=14.7 Hz, 1H), 4.66-4.64 (m, 1H), 4.62-4.49 (m, 4H), 4.37 (d, J=15.5 Hz, 1H), 3.92 (d, J=11.0 Hz, 1H), 3.82 (dd, J=11.0, 3.9 Hz, 1H), 3.77-3.70 (m, 2H), 3.48-3.40 (m, 3H), 2.49 (s, 3H), 2.44-2.35 (m, 1H), 2.34-2.21 (m, 3H), 2.17-2.07 (m, 2H), 1.82 (s, 1H), 1.68-1.57 (m, 3H), 1.51 (d, J=6.6 Hz, 3H), 1.44-1.30 (m, 16H), 1.05 (s, 9H). HRMS m/z [M+H]⁺ calcd for C₅₅H₇₄N₉O₆S⁺ 988.5477, found 988.5481.

### Example 134

### Synthesis of Intermediate 15

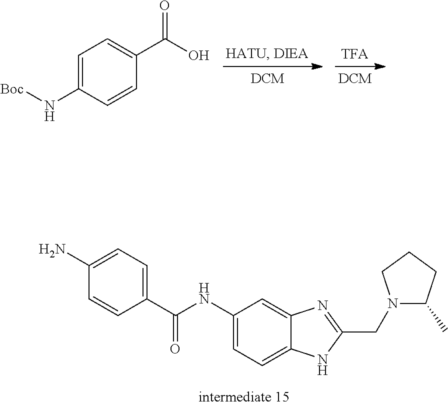 [SMILES: CC(C)(C)OC(=O)Nc1ccc(C(=O)O)cc1 ; C[C@H]1CCCN1Cc1nc2cc(NC(=O)c3ccc(N)cc3)ccc2[nH]1]

### Intermediate 15: (S)-4-amino-N-(2-((2-methylpyrrolidin-1-yl)methyl)-1H-benzo[d]imidazol-5-yl)benzamide

A solution of intermediate 8 (Moustakim et al., 2018) (100 mg, 0.43 mmol) was dissolved in DMF and treated with 4-((tert-Butoxycarbonyl)amino)benzoic acid (103 mg, 0.43 mmol), HATU (196 mg, 0.52 mmol) and DIEA (220 μL, 1.3 mmol). After being stirring 1 h at room temperature, the reaction mixture was poured into ice water, aqueous phase was extracted with ethyl acetate. The combined organic phase was washed with brine twice, dried and concentrated. The resulting residue was purified by silica gel flash chromatography to give the compound as yellow oil. The obtained oil was dissolved in 2 mL DCM, to the resulting solution was added 1 mL TFA. After being stirred for 1 h at room temperature, the reaction mixture was concentrated and the residue was purified by reverse phase C18 column (10%-100% methanol/0.1% TFA in water) to afford intermediate 15 as white solid in TFA salt form (135 mg, 68%). ¹H NMR (600 MHz, Methanol-d₄) δ 8.25 (d, J=2.0 Hz, 1H), 7.92-7.89 (m, 2H), 7.66 (d, J=8.8 Hz, 1H), 7.54 (dd, J=8.8, 2.0 Hz, 1H), 7.04-7.00 (m, 2H), 4.80 (d, J=14.7 Hz, 1H), 4.56 (d, J=14.6 Hz, 1H), 3.77-3.69 (m, 2H), 3.48-3.42 (m, 1H), 2.43-2.36 (m, 1H), 2.19-2.06 (m, 2H), 1.86-1.78 (m, 1H), 1.50 (d, J=6.5 Hz, 3H). MS (ESI): m/z 350.3 [M+H]⁺.

### Example 135

### Synthesis of LQ081-132

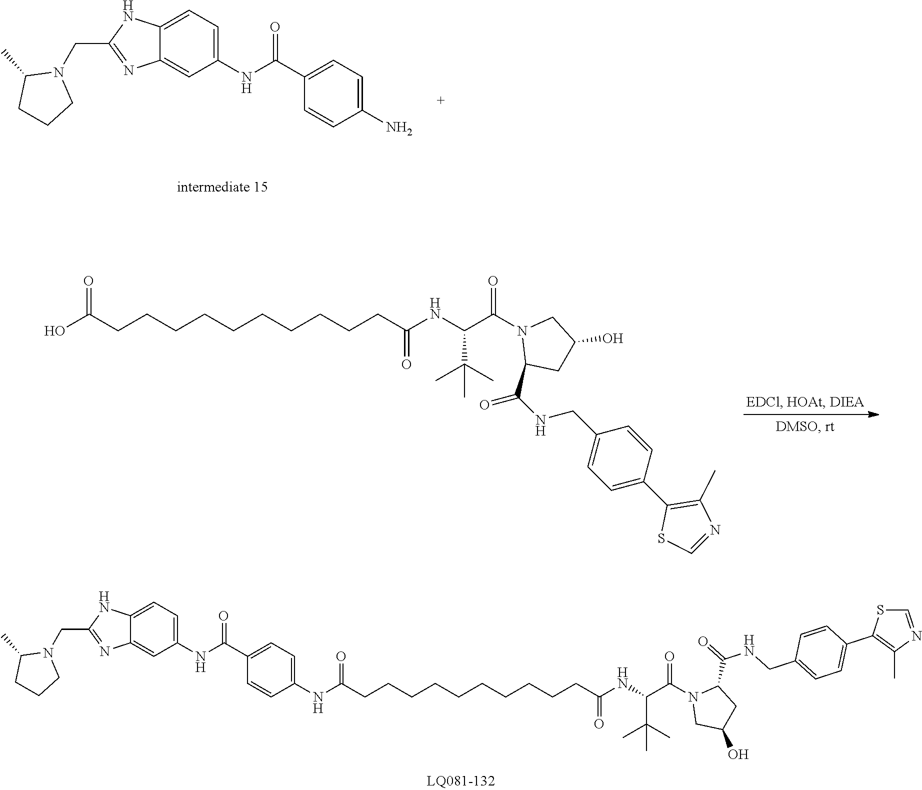

To a solution of Intermediate 15 (13 mg, 0.02 mmol) in DMSO (1 mL) were added 12-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-12-oxododecanoic acid (13.3 mg, 0.02 mmol, 1.0 equiv), EDCI (1-ethyl-3-(3-dimethylaminopropyl)carbodiimide) (5.8 mg, 0.03 mmol, 1.5 equiv), HOAt (1-hydroxy-7-azabenzo-triazole) (4.1 mg, 0.03 mmol, 1.5 equiv), and NMM (N-Methylmorpholine) (6.1 mg, 0.06 mmol, 3.0 equiv). After being stirred overnight at room temperature, the resulting mixture was purified by preparative HPLC (5%-60% acetonitrile/0.1% TFA in H₂O) to afford LQ081-132 as white solid in TFA salt form (19.2 mg, 80%). ¹H NMR (600 MHz, Methanol-d₄) δ 8.96 (s, 1H), 8.27 (d, J=1.9 Hz, 1H), 7.96 (d, J=8.7 Hz, 2H), 7.76 (d, J=8.6 Hz, 2H), 7.66 (d, J=8.7 Hz, 1H), 7.53 (dd, J=8.8, 2.0 Hz, 1H), 7.49-7.42 (m, 4H), 4.79 (d, J=14.6 Hz, 1H), 4.68-4.64 (m, 1H), 4.62-4.49 (m, 4H), 4.37 (d, J=15.5 Hz, 1H), 3.92 (d, J=10.9 Hz, 1H), 3.82 (dd, J=11.0, 3.9 Hz, 1H), 3.76-3.70 (m, 1H), 3.49-3.43 (m, 1H), 3.37 (s, 1H), 2.49 (s, 3H), 2.45-2.36 (m, 2H), 2.34-2.21 (m, 3H), 2.17-2.06 (m, 1H), 1.85-1.79 (m, 1H), 1.76-1.69 (m, 1H), 1.66-1.57 (m, 2H), 1.51 (d, J=6.5 Hz, 3H), 1.44-1.30 (m, 14H), 1.05 (s, 9H). HRMS m/z [M+H]⁺ calcd for C₅₄H₇₂N₉O₆S⁺ 974.5321, found 974.5312.

### Example 136

### Synthesis of LQ081-133

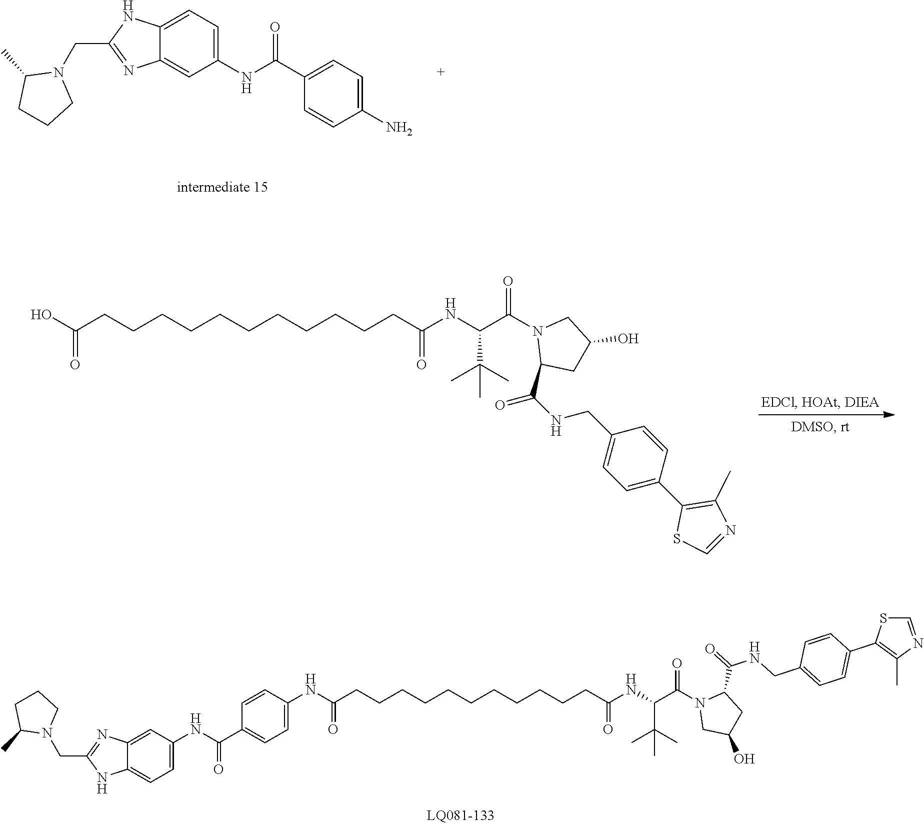

LQ081-133 was synthesized following the standard procedure for preparing LQ081-132 from intermediate 15 (13 mg, 0.02 mmol), (2S,4R)-1-((S)-2-(12-aminododecanamido)-3,3-dimethylbutanoyl)-4-hydroxy-N-(4-(4-methylthiazol-5-yl)benzyl)pyrrolidine-2-carboxamide (13.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ081-133 was obtained as white solid in TFA salt form (18.6 mg, 76%). ¹H NMR (600 MHz, Methanol-d₄) δ 8.93 (s, 1H), 8.26 (d, J=1.9 Hz, 1H), 7.96 (d, J=8.7 Hz, 2H), 7.76 (d, J=8.6 Hz, 2H), 7.66 (d, J=8.7 Hz, 1H), 7.53 (dd, J=8.8, 2.0 Hz, 1H), 7.50-7.41 (m, 4H), 4.78 (d, J=14.7 Hz, 1H), 4.67-4.64 (m, 1H), 4.62-4.50 (m, 4H), 4.37 (d, J=15.4 Hz, 1H), 3.92 (d, J=11.1 Hz, 1H), 3.82 (dd, J=11.0, 3.9 Hz, 1H), 3.76-3.70 (m, 2H), 3.49-3.42 (m, 1H), 2.49 (s, 3H), 2.45-2.36 (m, 2H), 2.34-2.20 (m, 3H), 2.18-2.06 (m, 1H), 1.85-1.78 (m, 1H), 1.76-1.70 (m, 2H), 1.66-1.57 (m, 2H), 1.50 (d, J=6.6 Hz, 3H), 1.44-1.28 (m, 17H), 1.05 (s, 9H). HRMS m/z [M+H]⁺ calcd for C₅₅H₇₄N₉O₆S⁺ 988.5477, found 988.5505.

### Example 137

### Synthesis of LQ081-146

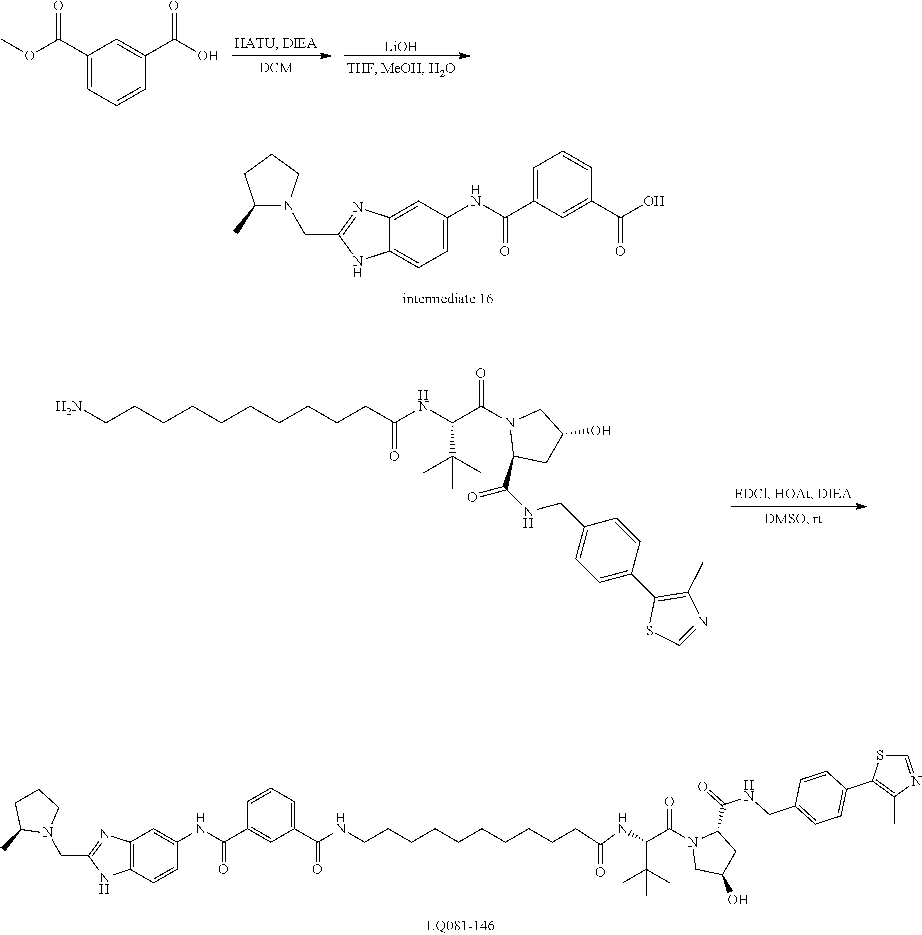

### Intermediate 16: (S)-3-((2-((2-methylpyrrolidin-1-yl)methyl)-1H-benzo[d]imidazol-5-yl)carbamoyl)benzoic acid

Intermediate 16 was synthesized according to the procedures for the preparation of intermediate 10 as a white solid in 67% yield. ¹H NMR (600 MHz, Methanol-d₄) δ 8.63 (s, 1H), 8.31 (d, J=1.9 Hz, 1H), 8.25 (d, J=7.8 Hz, 1H), 8.20 (d, J=7.9 Hz, 1H), 7.72-7.64 (m, 2H), 7.60 (dd, J=8.7, 2.0 Hz, 1H), 4.84 (d, J=14.6 Hz, 1H), 4.61 (d, J=14.6 Hz, 1H), 3.80-3.69 (m, 2H), 3.50-3.42 (m, 1H), 2.44-2.35 (m, 1H), 2.20-2.05 (m, 2H), 1.87-1.79 (m, 1H), 1.51 (d, J=6.5 Hz, 3H). MS (ESI): m/z 379.3 [M+H]⁺.

To a solution of Intermediate 16 (10 mg, 0.02 mmol) in DMSO (1 mL) were added (2S,4R)-1-((S)-2-(11-aminoundecanamido)-3,3-dimethylbutanoyl)-4-hydroxy-N-(4-(4-methylthiazol-5-yl)benzyl)pyrrolidine-2-carboxamide (14.3 mg, 0.02 mmol, 1.0 equiv), EDCI (1-ethyl-3-(3-dimethylaminopropyl)carbodiimide) (5.8 mg, 0.03 mmol, 1.5 equiv), HOAt (1-hydroxy-7-azabenzo-triazole) (4.1 mg, 0.03 mmol, 1.5 equiv), and NMM (N-Methylmorpholine) (6.1 mg, 0.06 mmol, 3.0 equiv). After being stirred overnight at room temperature, the resulting mixture was purified by preparative HPLC (5%-60% acetonitrile/0.1% TFA in H₂O) to afford LQ081-146 as white solid in TFA salt form (17.7 mg, 74%). ¹H NMR (600 MHz, Methanol-d₄) δ 9.06 (s, 1H), 8.43 (t, J=1.9 Hz, 1H), 8.33 (d, J=2.0 Hz, 1H), 8.12 (d, J=7.7 Hz, 1H), 8.03 (dt, J=7.7, 1.4 Hz, 1H), 7.69 (d, J=8.7 Hz, 1H), 7.64 (t, J=7.8 Hz, 1H), 7.58 (dd, J=8.8, 2.0 Hz, 1H), 7.50-7.42 (m, 4H), 4.84 (d, J=14.6 Hz, 1H), 4.66-4.63 (m, 1H), 4.62-4.49 (m, 4H), 4.37 (d, J=15.5 Hz, 1H), 3.92 (d, J=11.0 Hz, 1H), 3.81 (dd, J=10.9, 3.9 Hz, 1H), 3.77-3.71 (m, 2H), 3.49-3.39 (m, 3H), 2.50 (s, 3H), 2.43-2.36 (m, 1H), 2.33-2.20 (m, 3H), 2.18-2.06 (m, 2H), 1.87-1.79 (m, 1H), 1.69-1.57 (m, 3H), 1.51 (d, J=6.5 Hz, 3H), 1.45-1.30 (m, 14H), 1.04 (s, 9H). HRMS m/z [M+H]⁺ calcd for C₅₄H₇₂N₉O₆S⁺ 974.5321, found 974.5337.

### Example 138

### Synthesis of LQ081-147

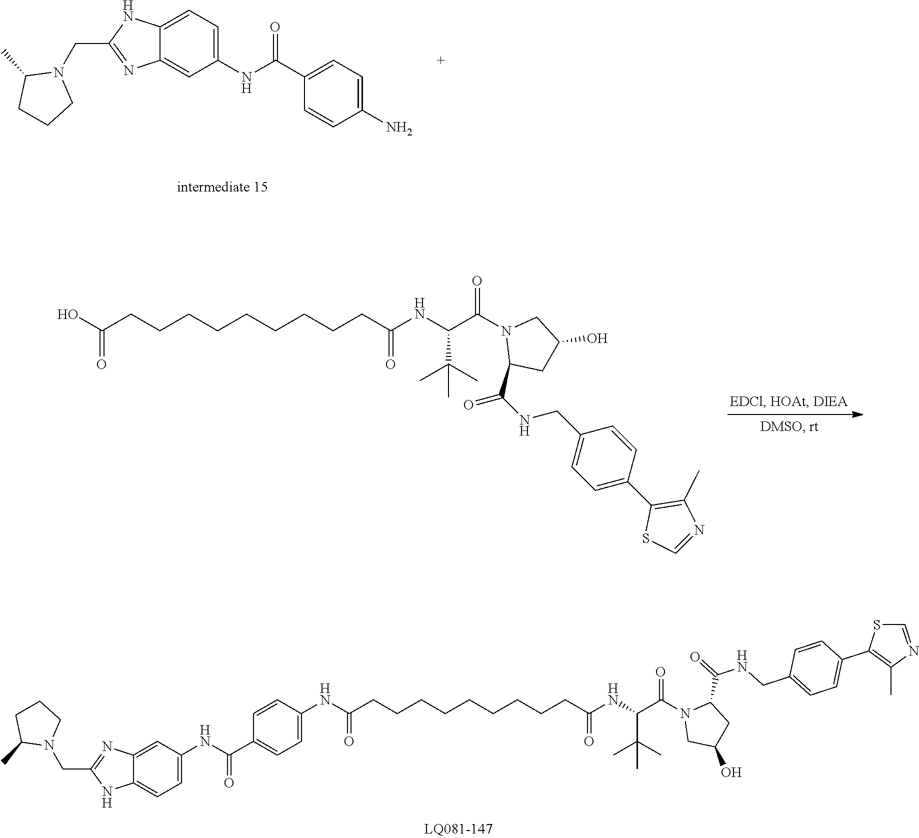

LQ081-147 was synthesized following the standard procedure for preparing LQ081-132 from intermediate 15 (13 mg, 0.02 mmol), 11-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-11-oxoundecanoic acid (13.1 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ081-147 was obtained as white solid in TFA salt form (16.4 mg, 69%). ¹H NMR (600 MHz, Methanol-d₄) δ 8.99 (s, 1H), 8.27 (d, J=1.9 Hz, 1H), 7.98-7.93 (m, 2H), 7.76 (d, J=8.7 Hz, 2H), 7.67 (d, J=8.7 Hz, 1H), 7.53 (dd, J=8.8, 2.0 Hz, 1H), 7.50-7.46 (m, 2H), 7.45-7.42 (m, 2H), 4.79 (d, J=14.5 Hz, 1H), 4.66-4.64 (m, 1H), 4.61-4.50 (m, 4H), 4.37 (d, J=15.5 Hz, 1H), 3.92 (d, J=11.1 Hz, 1H), 3.82 (dd, J=11.0, 3.9 Hz, 1H), 3.77-3.70 (m, 1H), 3.49-3.43 (m, 1H), 2.49 (s, 3H), 2.45-2.37 (m, 2H), 2.34-2.21 (m, 3H), 2.18-2.07 (m, 2H), 1.86-1.80 (m, 1H), 1.75-1.70 (m, 2H), 1.66-1.58 (m, 2H), 1.51 (d, J=6.5 Hz, 3H), 1.44-1.32 (m, 12H), 1.05 (s, 9H). HRMS m/z [M+H]⁺ calcd for C₅₃H₇₀N₉O₆S⁺ 960.5164, found 960.5212.

### Example 139

### Synthesis of LQ081-150

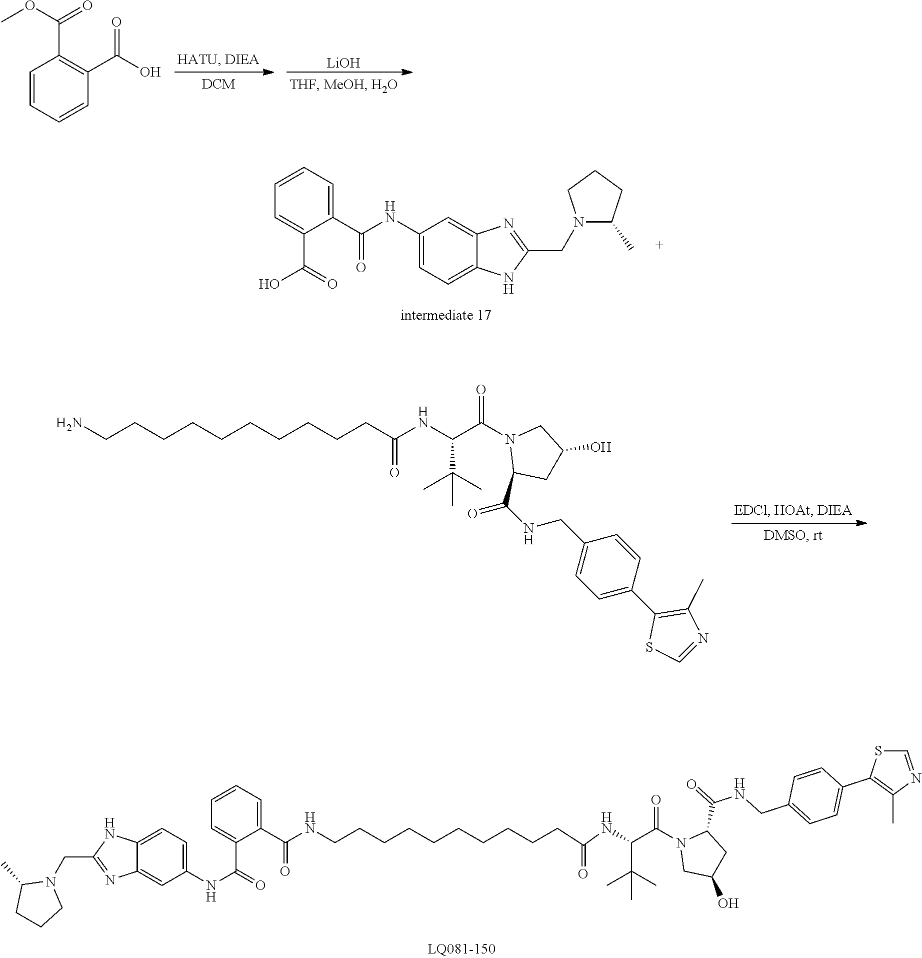

### Intermediate 17: (S)-2-((2-((2-methylpyrrolidin-1-yl)methyl)-1H-benzo[d]imidazol-5-yl)carbamoyl)benzoic acid

Intermediate 17 was synthesized according to the procedures for the preparation of intermediate 10 as a white solid in 77% yield. ¹H NMR (600 MHz, Methanol-d₄) δ 8.01-7.95 (m, 2H), 7.93-7.87 (m, 2H), 7.80-7.75 (m, 2H), 7.41 (dd, J=8.6, 1.9 Hz, 1H), 4.84 (d, J=14.6 Hz, 1H), 4.61 (d, J=14.6 Hz, 1H), 3.82-3.74 (m, 2H), 3.53-3.46 (m, 1H), 2.45-2.36 (m, 1H), 2.21-2.05 (m, 2H), 1.88-1.79 (m, 1H), 1.52 (d, J=6.5 Hz, 3H). MS (ESI): m/z 379.2 [M+H]⁺.

To a solution of Intermediate 17 (10 mg, 0.02 mmol) in DMSO (1 mL) were added (2S,4R)-1-((S)-2-(11-aminoundecanamido)-3,3-dimethylbutanoyl)-4-hydroxy-N-(4-(4-methylthiazol-5-yl)benzyl)pyrrolidine-2-carboxamide (14.4 mg, 0.02 mmol, 1.0 equiv), EDCI (1-ethyl-3-(3-dimethylaminopropyl)carbodiimide) (5.8 mg, 0.03 mmol, 1.5 equiv), HOAt (1-hydroxy-7-azabenzo-triazole) (4.1 mg, 0.03 mmol, 1.5 equiv), and NMM (N-Methylmorpholine) (6.1 mg, 0.06 mmol, 3.0 equiv). After being stirred overnight at room temperature, the resulting mixture was purified by preparative HPLC (5%-60% acetonitrile/0.1% TFA in H₂O) to afford LQ081-150 as white solid in TFA salt form (18.2 mg, 76%). ¹H NMR (600 MHz, Methanol-d₄) δ 9.05 (s, 1H), 8.31 (s, 1H), 7.70 (d, J=6.0 Hz, 1H), 7.66-7.58 (m, 3H), 7.51-7.42 (m, 6H), 4.80 (d, J=14.8 Hz, 1H), 4.66-4.63 (m, 1H), 4.60-4.50 (m, 4H), 4.38 (d, J=15.5 Hz, 1H), 3.92 (d, J=11.0 Hz, 1H), 3.82 (dd, J=11.0, 3.7 Hz, 1H), 3.76-3.70 (m, 2H), 3.49-3.42 (m, 1H), 3.35-3.33 (m, 2H), 2.50 (s, 3H), 2.43-2.36 (m, 1H), 2.32-2.20 (m, 3H), 2.18-2.07 (m, 1H), 1.85-1.78 (m, 1H), 1.65-1.52 (m, 4H), 1.50 (d, J=6.5 Hz, 3H), 1.41-1.19 (m, 14H), 1.05 (s, 9H). HRMS m/z [M+H]⁺ calcd for C₅₄H₇₂N₉O₆S⁺ 974.5321, found 974.5343.

### Synthesis of LQ081-158

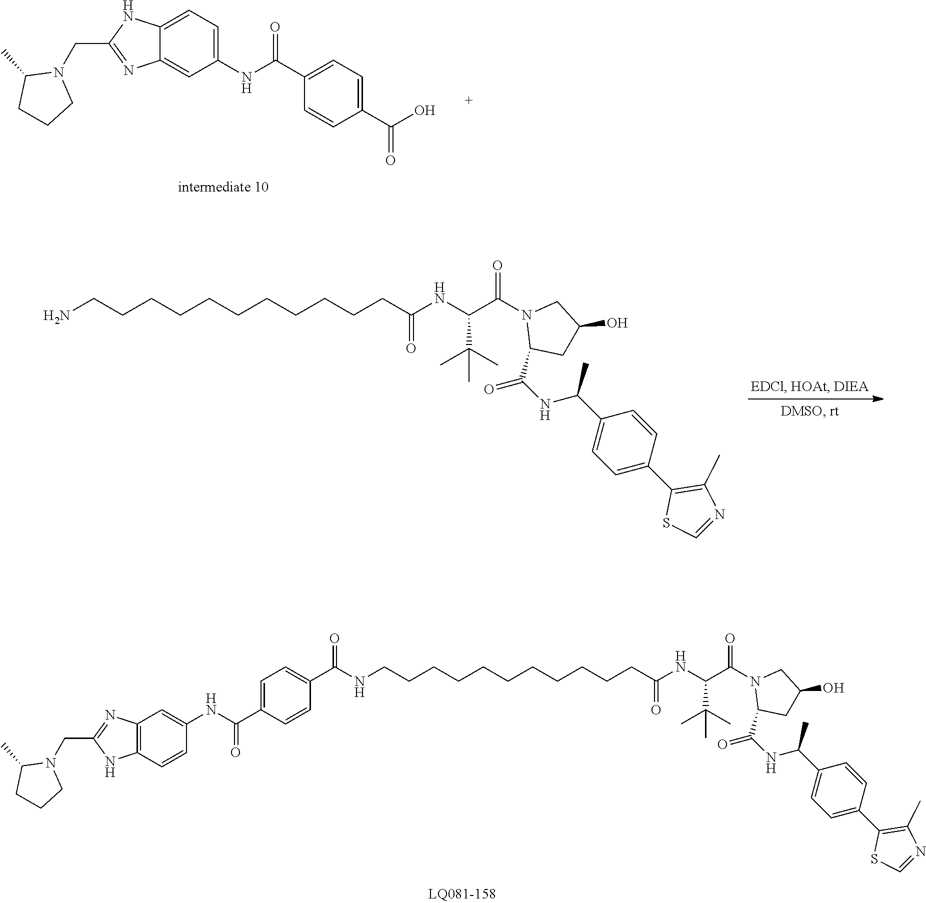

LQ081-158 was synthesized following the standard procedure for preparing LQ076-105 from intermediate 10 (10 mg, 0.02 mmol), (2R,4S)-1-((S)-2-(12-aminododecanamido)-3,3-dimethylbutanoyl)-4-hydroxy-N—((S)-1-(4-(4-methylthiazol-5-yl)phenyl)ethyl)pyrrolidine-2-carboxamide (14.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ081-158 was obtained as white solid in TFA salt form (19.1 mg, 78%). ¹H NMR (600 MHz, Methanol-d₄) δ 8.99 (s, 1H), 8.30 (d, J=2.0 Hz, 1H), 8.08-8.03 (m, 2H), 7.99-7.94 (m, 2H), 7.68 (d, J=8.7 Hz, 1H), 7.60-7.55 (m, 1H), 7.54-7.50 (m, 2H), 7.49-7.45 (m, 2H), 5.07-5.01 (m, 1H), 4.81 (d, J=14.7 Hz, 1H), 4.60-4.54 (m, 2H), 4.52-4.48 (m, 1H), 4.48-4.44 (m, 1H), 3.99-3.93 (m, 1H), 3.78-3.67 (m, 3H), 3.50-3.43 (m, 1H), 3.41 (t, J=7.2 Hz, 2H), 2.51 (s, 3H), 2.44-2.36 (m, 1H), 2.34-2.27 (m, 1H), 2.25-2.06 (m, 5H), 1.87-1.78 (m, 1H), 1.69-1.53 (m, 4H), 1.51 (d, J=6.5 Hz, 3H), 1.46 (d, J=7.0 Hz, 3H), 1.43-1.25 (m, 14H), 1.08 (s, 9H). HRMS m/z [M+H]⁺ calcd for C₅₆H₇₆N₉O₆S⁺ 1002.5634, found 1002.5642.

### Example 141

### Synthesis of LQ086-31

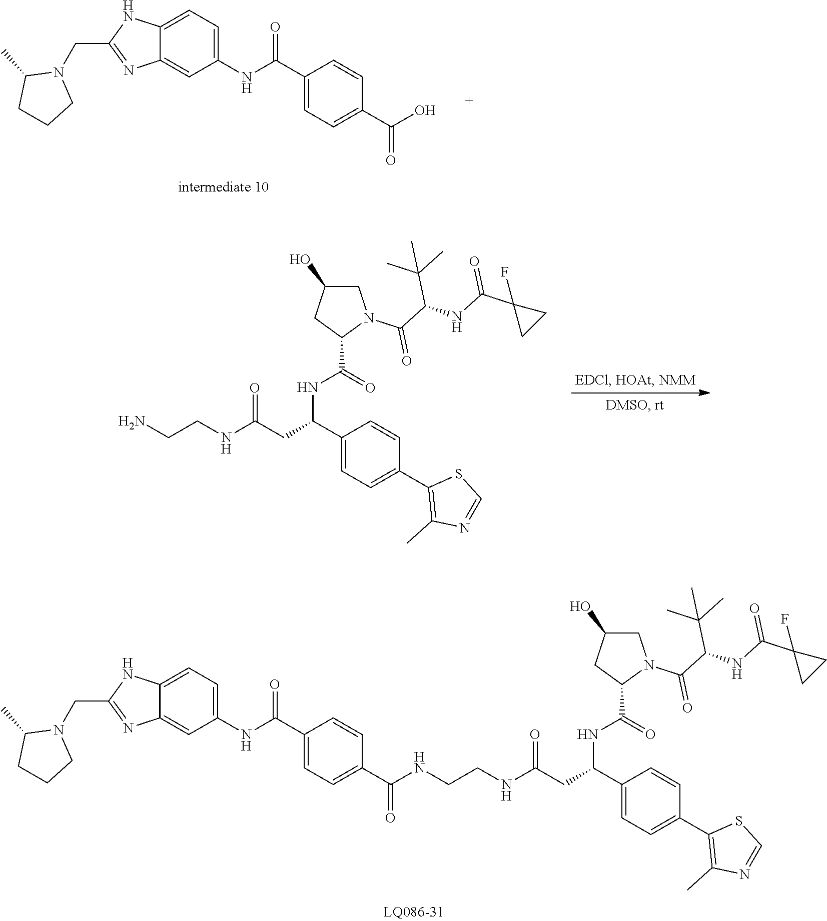 [SMILES: C[C@H]1CCCN1Cc1nc2cc(NC(=O)c3ccc(C(=O)O)cc3)ccc2[nH]1 ; Cc1ncsc1-c1ccc([C@H](CC(=O)NCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1]

LQ086-31 was synthesized following the standard procedure for preparing LQ076-105 from intermediate 10 (10 mg, 0.02 mmol), (2S,4R)—N—((S)-3-((2-aminoethyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (14.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ086-31 was obtained as white solid in TFA salt form (16.7 mg, 69%). ¹H NMR (600 MHz, Methanol-d₄) δ 8.94 (s, 1H), 8.30 (d, J=1.9 Hz, 1H), 8.05-8.00 (m, 2H), 7.95-7.91 (m, 2H), 7.69 (d, J=8.7 Hz, 1H), 7.57 (dd, J=8.7, 2.0 Hz, 1H), 7.48-7.44 (m, 2H), 7.42-7.37 (m, 2H), 5.40 (t, J=7.1 Hz, 1H), 4.81 (d, J=14.7 Hz, 1H), 4.78-4.72 (m, 1H), 4.66-4.55 (m, 2H), 4.50-4.45 (m, 1H), 3.90-3.85 (m, 1H), 3.82-3.71 (m, 3H), 3.54-3.37 (m, 6H), 2.87 (dd, J=14.4, 7.0 Hz, 1H), 2.80 (dd, J=14.3, 7.3 Hz, 1H), 2.47 (s, 3H), 2.44-2.36 (m, 1H), 2.26-2.20 (m, 1H), 2.18-2.06 (m, 1H), 2.01-1.95 (m, 1H), 1.87-1.78 (m, 1H), 1.51 (d, J=6.5 Hz, 3H), 1.40-1.20 (m, 4H), 1.07 (s, 9H). HRMS m/z [M+H]⁺ calcd for C₅₁H₆₂FN₁₀O₇S⁺ 977.4502, found 977.4488.

### Example 142

### Synthesis of LQ086-32

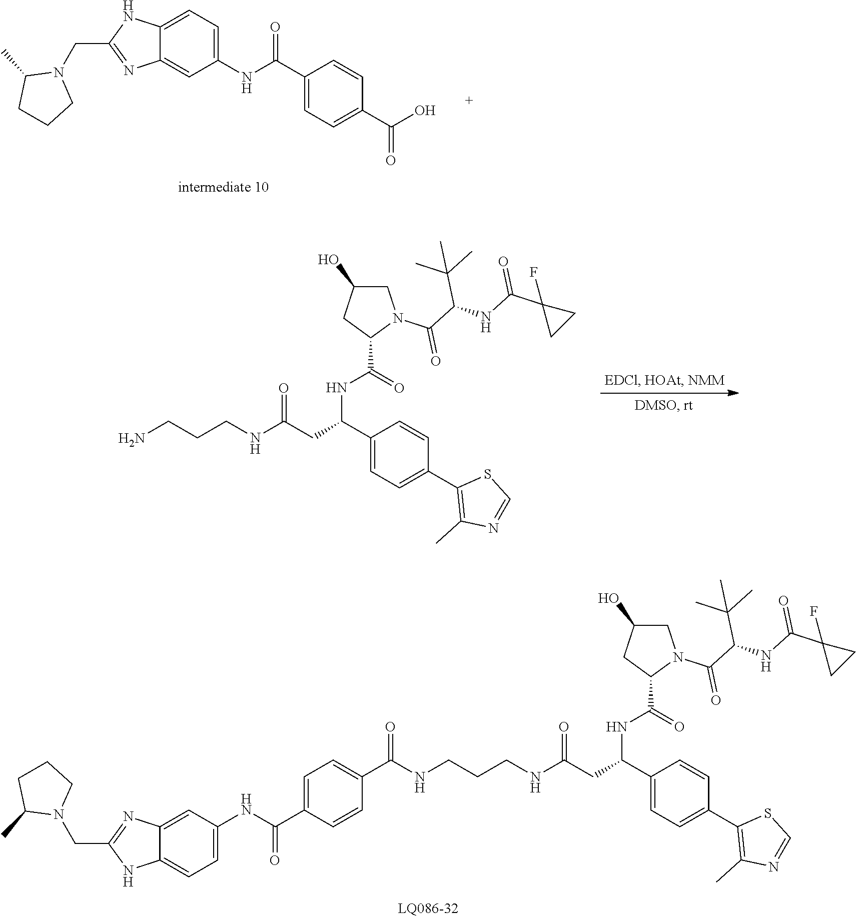 [SMILES: C[C@H]1CCCN1Cc1nc2cc(NC(=O)c3ccc(C(=O)O)cc3)ccc2[nH]1 ; Cc1ncsc1-c1ccc([C@H](CC(=O)NCCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1]

LQ086-32 was synthesized following the standard procedure for preparing LQ076-105 from intermediate 10 (10 mg, 0.02 mmol), (2S,4R)—N—((S)-3-((3-aminopropyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (14.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ086-32 was obtained as white solid in TFA salt form (16.2 mg, 66%). ¹H NMR (600 MHz, Methanol-d₄) δ 8.80 (s, 1H), 8.18 (d, J=1.9 Hz, 1H), 7.96-7.91 (m, 2H), 7.86-7.80 (m, 2H), 7.57 (d, J=8.7 Hz, 1H), 7.44 (dd, J=8.7, 2.0 Hz, 1H), 7.41-7.32 (m, 4H), 5.26 (dd, J=8.2, 6.2 Hz, 1H), 4.68 (d, J=14.7 Hz, 1H), 4.64 (dd, J=9.4, 1.2 Hz, 1H), 4.51 (dd, J=9.3, 7.6 Hz, 1H), 4.45 (d, J=14.6 Hz, 1H), 4.37-4.32 (m, 1H), 3.76-3.71 (m, 1H), 3.70-3.59 (m, 3H), 3.39-3.31 (m, 1H), 3.21-3.01 (m, 5H), 2.79 (dd, J=14.1, 6.2 Hz, 1H), 2.69 (dd, J=14.2, 8.3 Hz, 1H), 2.36 (s, 3H), 2.33-2.24 (m, 1H), 2.13-2.08 (m, 1H), 2.07-1.95 (m, 1H), 1.90-1.83 (m, 1H), 1.76-1.66 (m, 1H), 1.62-1.54 (m, 2H), 1.40 (d, J=6.5 Hz, 3H), 1.30-1.14 (m, 4H), 0.96 (s, 9H). HRMS m/z [M+H]⁺ calcd for C₅₂H₆₄FN₁₀O₇S⁺ 991.4659, found 991.4624.

### Example 143

### Synthesis of LQ086-33

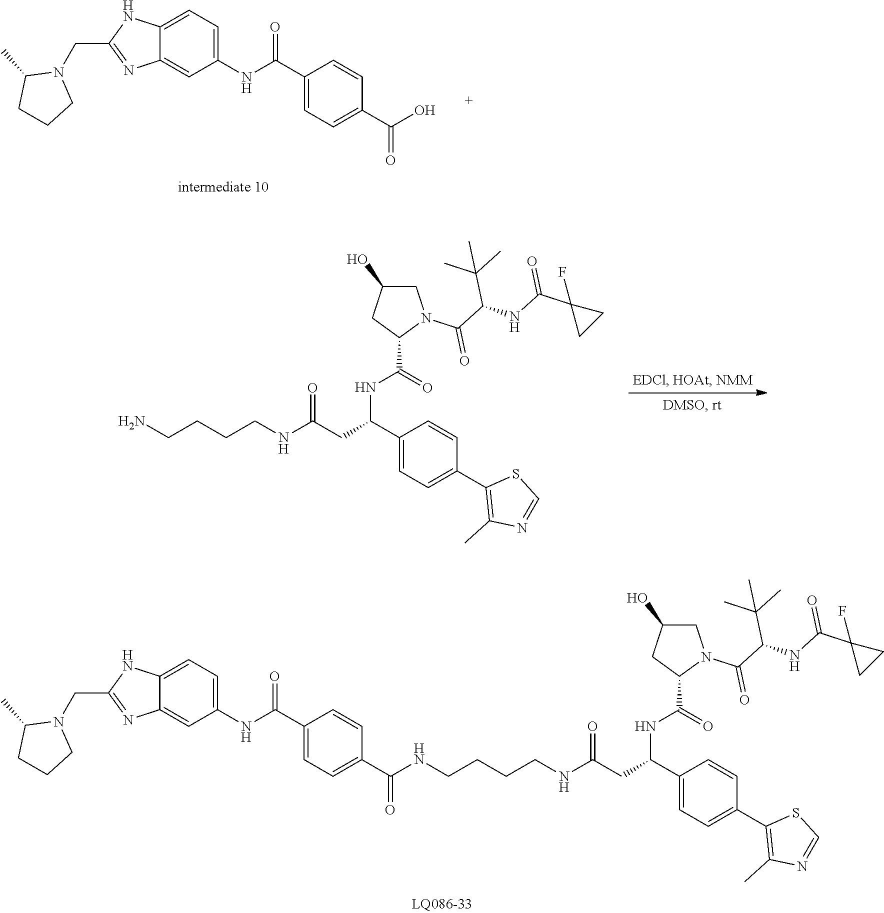 [SMILES: C[C@H]1CCCN1Cc1nc2cc(NC(=O)c3ccc(C(=O)O)cc3)ccc2[nH]1 ; Cc1ncsc1-c1ccc([C@H](CC(=O)NCCCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1]

LQ086-33 was synthesized following the standard procedure for preparing LQ076-105 from intermediate 10 (10 mg, 0.02 mmol), (2S,4R)—N—((S)-3-((4-aminobutyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (14.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ086-33 was obtained as white solid in TFA salt form (17.2 mg, 70%). ¹H NMR (600 MHz, Methanol-d₄) δ 8.81 (s, 1H), 8.18 (d, J=2.0 Hz, 1H), 7.94-7.90 (m, 2H), 7.85-7.80 (m, 2H), 7.57 (d, J=8.8 Hz, 1H), 7.45 (dd, J=8.8, 2.0 Hz, 1H), 7.38-7.31 (m, 4H), 5.23 (dd, J=8.2, 6.2 Hz, 1H), 4.69 (d, J=14.6 Hz, 1H), 4.64 (d, J=8.7 Hz, 1H), 4.52-4.42 (m, 2H), 4.38-4.32 (m, 1H), 3.76-3.71 (m, 1H), 3.69-3.59 (m, 3H), 3.39-3.31 (m, 1H), 3.25 (t, J=6.6 Hz, 2H), 3.14-3.00 (m, 2H), 2.75 (dd, J=14.1, 6.2 Hz, 1H), 2.66 (dd, J=14.1, 8.3 Hz, 1H), 2.36 (s, 3H), 2.32-2.25 (m, 1H), 2.13-2.07 (m, 1H), 2.07-1.95 (m, 2H), 1.89-1.83 (m, 1H), 1.76-1.66 (m, 1H), 1.45-1.36 (m, 7H), 1.30-1.13 (m, 4H), 0.96 (s, 9H). HRMS m/z [M+H]⁺ calcd for C₅₃H₆₆FN₁₀O₇S⁺ 1005.4815, found 1005.4822.

### Example 144

### Synthesis of LQ086-34

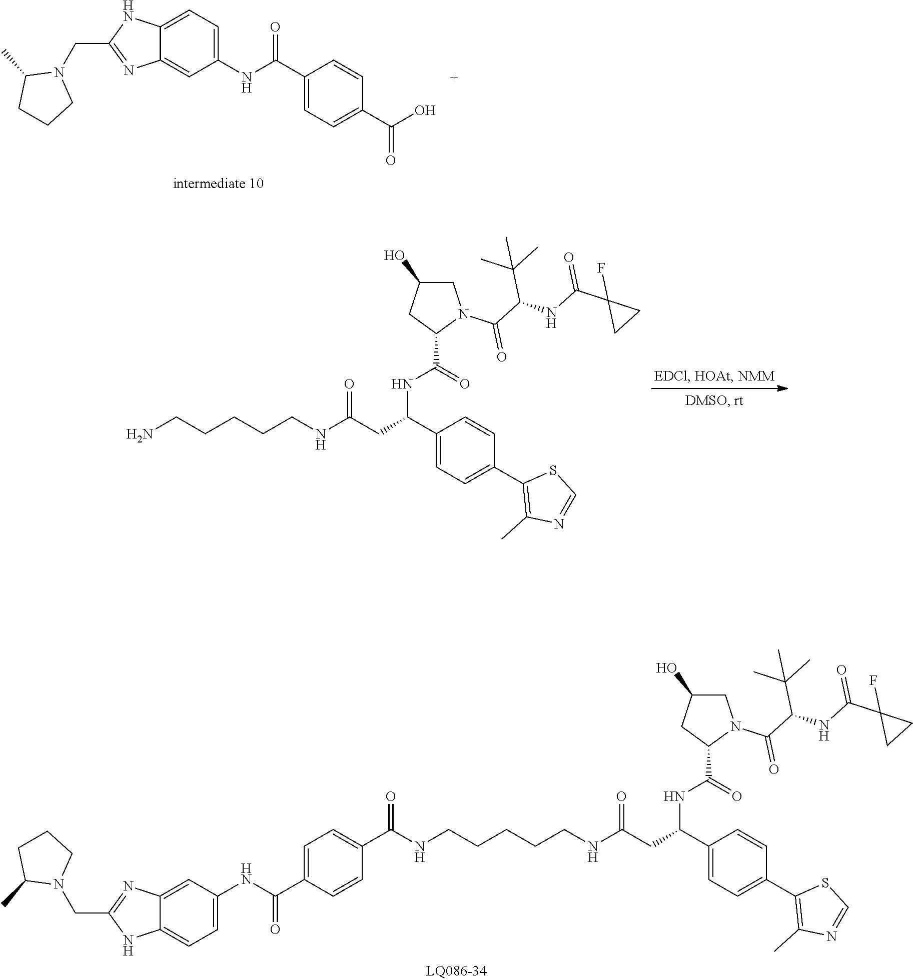 [SMILES: C[C@H]1CCCN1Cc1nc2cc(NC(=O)c3ccc(C(=O)O)cc3)ccc2[nH]1 ; Cc1ncsc1-c1ccc([C@H](CC(=O)NCCCCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1]

LQ086-34 was synthesized following the standard procedure for preparing LQ076-105 from intermediate 10 (10 mg, 0.02 mmol), (2S,4R)—N—((S)-3-((5-aminopentyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (15.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ086-34 was obtained as white solid in TFA salt form (17.6 mg, 71%). ¹H NMR (600 MHz, Methanol-d₄) δ 8.92 (s, 1H), 8.29 (d, J=2.0 Hz, 1H), 8.08-8.03 (m, 2H), 7.98-7.93 (m, 2H), 7.67 (d, J=8.8 Hz, 1H), 7.55 (dd, J=8.7, 2.0 Hz, 1H), 7.52-7.44 (m, 4H), 5.33 (dd, J=8.2, 6.3 Hz, 1H), 4.81 (d, J=14.7 Hz, 1H), 4.75 (dd, J=9.3, 1.3 Hz, 1H), 4.63-4.55 (m, 2H), 4.48-4.44 (m, 1H), 3.87-3.82 (m, 1H), 3.80-3.71 (m, 3H), 3.51-3.43 (m, 1H), 3.38-3.34 (m, 2H), 3.22-3.15 (m, 1H), 3.15-3.08 (m, 1H), 2.85 (dd, J=14.2, 6.3 Hz, 1H), 2.75 (dd, J=14.2, 8.2 Hz, 1H), 2.48 (s, 3H), 2.44-2.37 (m, 1H), 2.26-2.19 (m, 1H), 2.19-2.06 (m, 2H), 2.03-1.94 (m, 1H), 1.87-1.79 (m, 1H), 1.64-1.56 (m, 2H), 1.51 (d, J=6.5 Hz, 3H), 1.49-1.42 (m, 2H), 1.41-1.26 (m, 6H), 1.07 (s, 9H). HRMS m/z [M+H]⁺ calcd for C₅₄H₆₈FN₁₀O₇S⁺ 1019.4972, found 1019.4964.

### Example 145

### Synthesis of LQ086-35

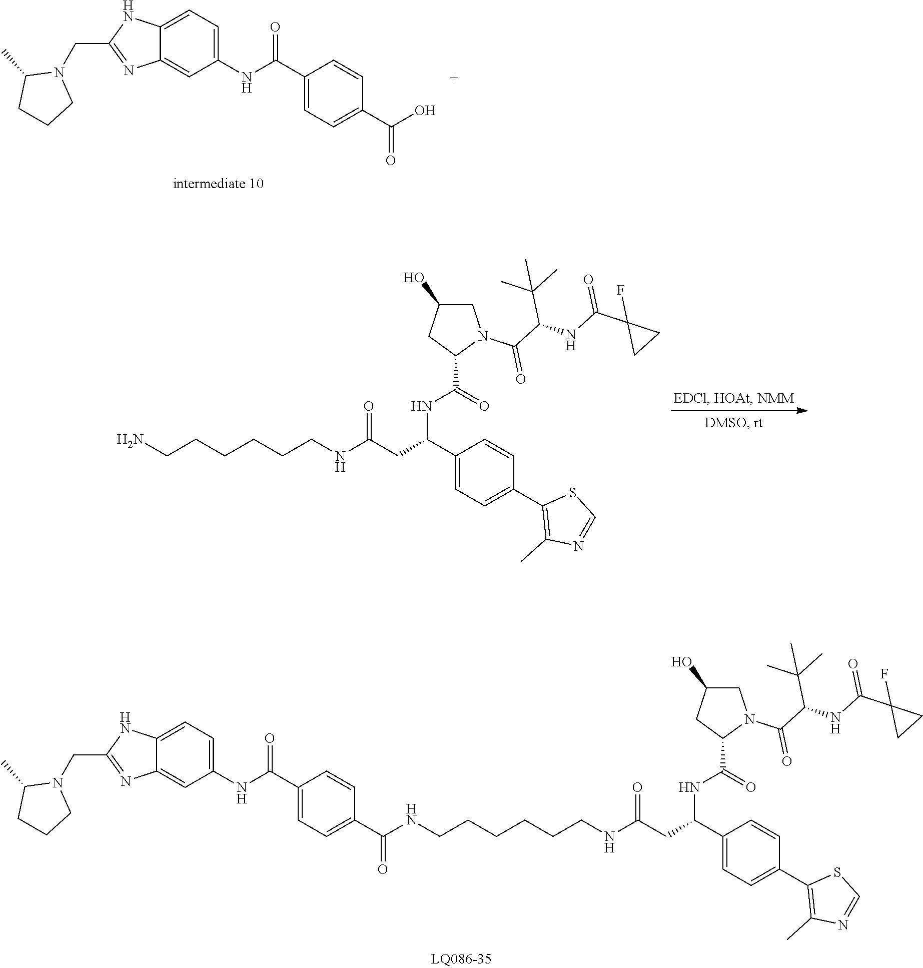 [SMILES: C[C@H]1CCCN1Cc1nc2cc(NC(=O)c3ccc(C(=O)O)cc3)ccc2[nH]1 ; Cc1ncsc1-c1ccc([C@H](CC(=O)NCCCCCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1]

LQ086-35 was synthesized following the standard procedure for preparing LQ076-105 from intermediate 10 (10 mg, 0.02 mmol), (2S,4R)—N—((S)-3-((6-aminohexyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (15.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ086-35 was obtained as white solid in TFA salt form (18.1 mg, 72%). ¹H NMR (600 MHz, Methanol-d₄) δ 8.93 (s, 1H), 8.30 (d, J=2.0 Hz, 1H), 8.08-8.03 (m, 2H), 7.98-7.94 (m, 2H), 7.68 (d, J=8.7 Hz, 1H), 7.56 (dd, J=8.8, 2.0 Hz, 1H), 7.52-7.44 (m, 4H), 5.33 (dd, J=8.4, 6.0 Hz, 1H), 4.81 (d, J=14.6 Hz, 1H), 4.75 (dd, J=9.3, 1.3 Hz, 1H), 4.63-4.55 (m, 2H), 4.48-4.43 (m, 1H), 3.87-3.82 (m, 1H), 3.80-3.70 (m, 3H), 3.51-3.43 (m, 1H), 3.36 (t, J=7.1 Hz, 2H), 3.19-3.13 (m, 1H), 3.12-3.06 (m, 1H), 2.86 (dd, J=14.1, 5.9 Hz, 1H), 2.77 (dd, J=14.1, 8.4 Hz, 1H), 2.49 (s, 3H), 2.44-2.36 (m, 1H), 2.26-2.19 (m, 1H), 2.18-2.06 (m, 2H), 2.01-1.95 (m, 1H), 1.87-1.79 (m, 1H), 1.64-1.55 (m, 2H), 1.51 (d, J=6.5 Hz, 3H), 1.45-1.22 (m, 10H), 1.07 (s, 9H). HRMS m/z [M+H]⁺ calcd for C₅₅H₇₀FN₁₀O₇S⁺ 1033.5128, found 1033.5138.

### Example 146

### Synthesis of LQ086-36

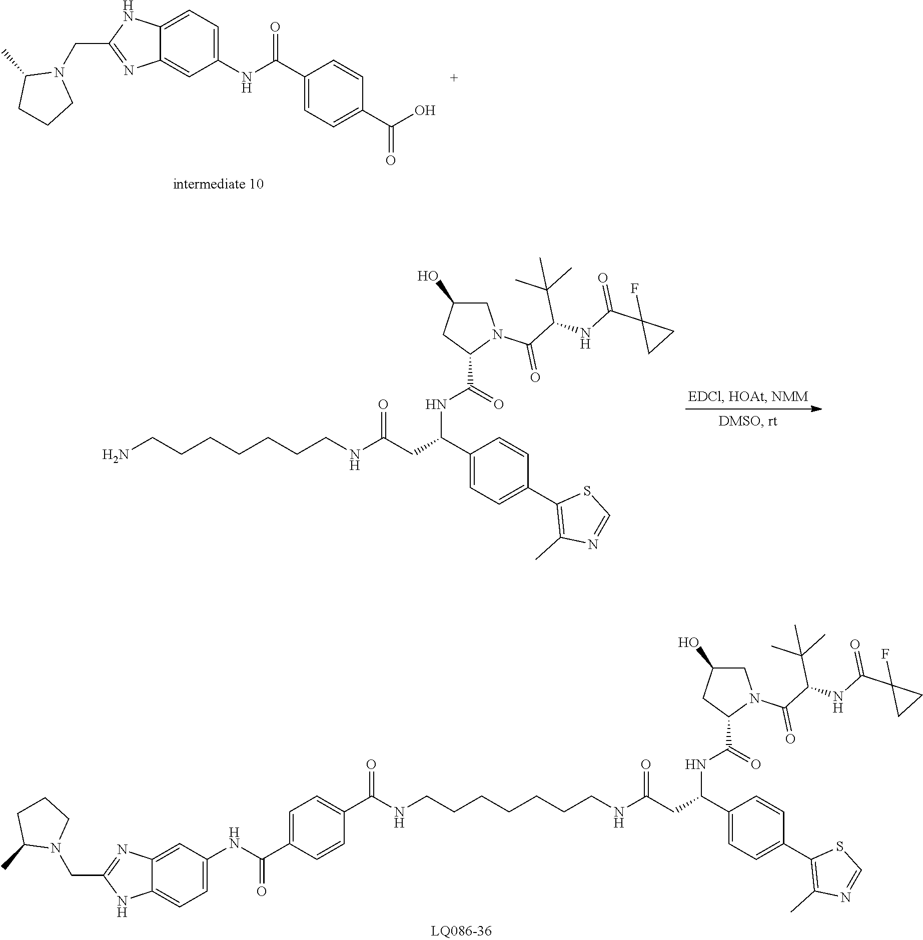 [SMILES: C[C@H]1CCCN1Cc1nc2cc(NC(=O)c3ccc(C(=O)O)cc3)ccc2[nH]1 ; Cc1ncsc1-c1ccc([C@H](CC(=O)NCCCCCCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1]

LQ086-36 was synthesized following the standard procedure for preparing LQ076-105 from intermediate 10 (10 mg, 0.02 mmol), (2S,4R)—N—((S)-3-((7-aminoheptyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (15.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ086-36 was obtained as white solid in TFA salt form (16.3 mg, 64%). ¹H NMR (600 MHz, Methanol-d₄) δ 8.83 (s, 1H), 8.18 (d, J=1.9 Hz, 1H), 7.94 (d, J=8.4 Hz, 2H), 7.85 (d, J=8.5 Hz, 2H), 7.56 (d, J=8.8 Hz, 1H), 7.45-7.42 (m, 1H), 7.35 (s, 4H), 5.21 (dd, J=8.4, 6.0 Hz, 1H), 4.68 (d, J=14.6 Hz, 1H), 4.63 (d, J=9.5 Hz, 1H), 4.50-4.42 (m, 2H), 4.36-4.33 (m, 1H), 3.75-3.70 (m, 1H), 3.68-3.60 (m, 3H), 3.39-3.32 (m, 1H), 3.28-3.24 (m, 2H), 3.07-2.99 (m, 1H), 2.98-2.93 (m, 1H), 2.74 (dd, J=14.1, 6.0 Hz, 1H), 2.64 (dd, J=14.1, 8.4 Hz, 1H), 2.38 (s, 3H), 2.32-2.24 (m, 1H), 2.13-2.07 (m, 1H), 2.07-1.95 (m, 2H), 1.89-1.83 (m, 1H), 1.75-1.67 (m, 1H), 1.48 (p, J=7.2 Hz, 2H), 1.40 (d, J=6.5 Hz, 3H), 1.30-1.16 (m, 10H), 1.13-1.06 (m, 2H), 0.96 (s, 9H). HRMS m/z [M+H]⁺ calcd for C₅₆H₇₂FN₁₀O₇S⁺ 1047.5285, found 1047.5291.

### Example 147

### Synthesis of LQ086-38

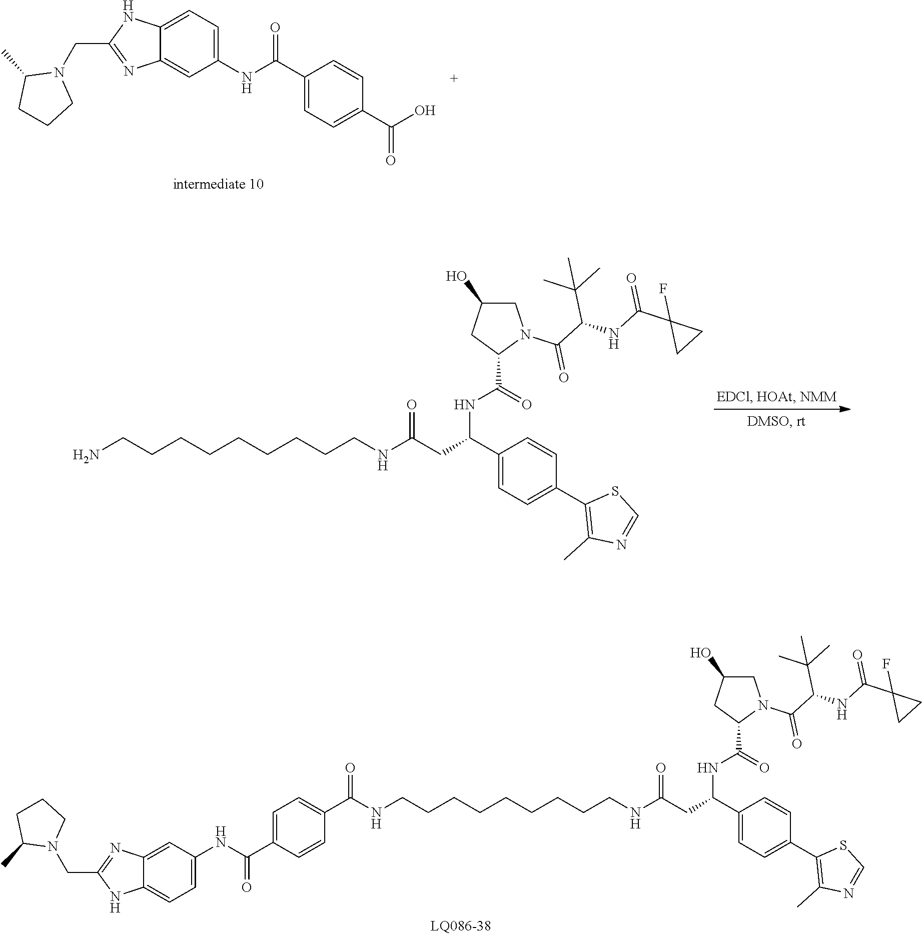 [SMILES: C[C@H]1CCCN1Cc1nc2cc(NC(=O)c3ccc(C(=O)O)cc3)ccc2[nH]1 ; Cc1ncsc1-c1ccc([C@H](CC(=O)NCCCCCCCCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1]

LQ086-38 was synthesized following the standard procedure for preparing LQ076-105 from intermediate 10 (10 mg, 0.02 mmol), (2S,4R)—N—((S)-3-((9-aminononyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (16.4 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ086-38 was obtained as white solid in TFA salt form (19.1 mg, 73%). ¹H NMR (600 MHz, Methanol-d₄) δ 8.84 (s, 1H), 8.18 (d, J=1.9 Hz, 1H), 7.94 (d, J=8.4 Hz, 2H), 7.85 (d, J=8.4 Hz, 2H), 7.56 (d, J=8.7 Hz, 1H), 7.44 (dd, J=8.7, 2.0 Hz, 1H), 7.41-7.33 (m, 4H), 5.21 (dd, J=8.5, 5.9 Hz, 1H), 4.69 (d, J=14.6 Hz, 1H), 4.64 (d, J=9.3 Hz, 1H), 4.51-4.43 (m, 2H), 4.37-4.32 (m, 1H), 3.75-3.70 (m, 1H), 3.69-3.59 (m, 3H), 3.39-3.31 (m, 1H), 3.28 (t, J=7.2 Hz, 2H), 3.05-2.97 (m, 1H), 2.97-2.89 (m, 1H), 2.74 (dd, J=14.0, 5.9 Hz, 1H), 2.67-2.61 (m, 1H), 2.38 (s, 3H), 2.32-2.24 (m, 1H), 2.13-2.07 (m, 1H), 2.07-1.95 (m, 2H), 1.90-1.83 (m, 1H), 1.76-1.66 (m, 1H), 1.50 (p, J=7.2 Hz, 2H), 1.39 (d, J=6.5 Hz, 3H), 1.31-1.09 (m, 14H), 1.07-1.00 (m, 2H), 0.96 (s, 9H). HRMS m/z [M+H]⁺ calcd for C₅₅H₇₆FN₁₀O₇S⁺ 1075.5598, found 1075.5607.

### Example 148

### Synthesis of LQ086-40

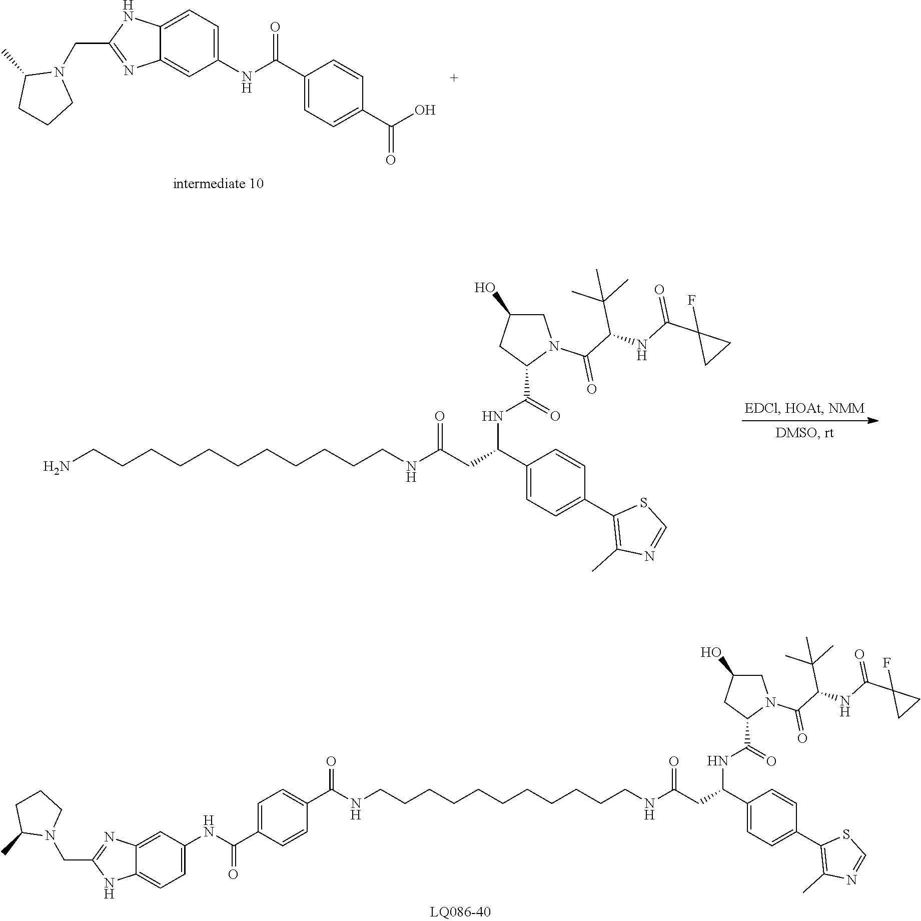 [SMILES: C[C@H]1CCCN1Cc1nc2cc(NC(=O)c3ccc(C(=O)O)cc3)ccc2[nH]1 ; Cc1ncsc1-c1ccc([C@H](CC(=O)NCCCCCCCCCCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1]

LQ086-40 was synthesized following the standard procedure for preparing LQ076-105 from intermediate 10 (10 mg, 0.02 mmol), (2S,4R)—N—((S)-3-((11-aminoundecyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (16.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ086-40 was obtained as white solid in TFA salt form (20 mg, 75%). ¹H NMR (600 MHz, Methanol-d₄) δ 8.83 (s, 1H), 8.17 (d, J=2.0 Hz, 1H), 7.94 (d, J=8.4 Hz, 2H), 7.86 (d, J=8.4 Hz, 2H), 7.56 (d, J=8.7 Hz, 1H), 7.44 (dd, J=8.7, 2.0 Hz, 1H), 7.41-7.32 (m, 4H), 5.21 (dd, J=8.5, 5.8 Hz, 1H), 4.68 (d, J=14.7 Hz, 1H), 4.64 (d, J=9.3 Hz, 1H), 4.51-4.43 (m, 2H), 4.37-4.32 (m, 1H), 3.75-3.70 (m, 1H), 3.69-3.60 (m, 3H), 3.39-3.32 (m, 1H), 3.30 (t, J=7.2 Hz, 2H), 3.04-2.98 (m, 1H), 2.96-2.89 (m, 1H), 2.74 (dd, J=14.0, 5.8 Hz, 1H), 2.64 (dd, J=14.0, 8.6 Hz, 1H), 2.38 (s, 3H), 2.32-2.25 (m, 1H), 2.13-2.07 (m, 1H), 2.06-1.95 (m, 2H), 1.89-1.83 (m, 1H), 1.75-1.67 (m, 1H), 1.53 (p, J=7.3 Hz, 2H), 1.39 (d, J=6.5 Hz, 3H), 1.34-1.07 (m, 16H), 1.06-0.99 (m, 2H), 0.96 (s, 9H). HRMS m/z [M+H]⁺ calcd for C₆₀H₈₀FN₁₀O₇S⁺ 1103.5911, found 1103.5898.

### Example 149

### Synthesis of LQ086-41

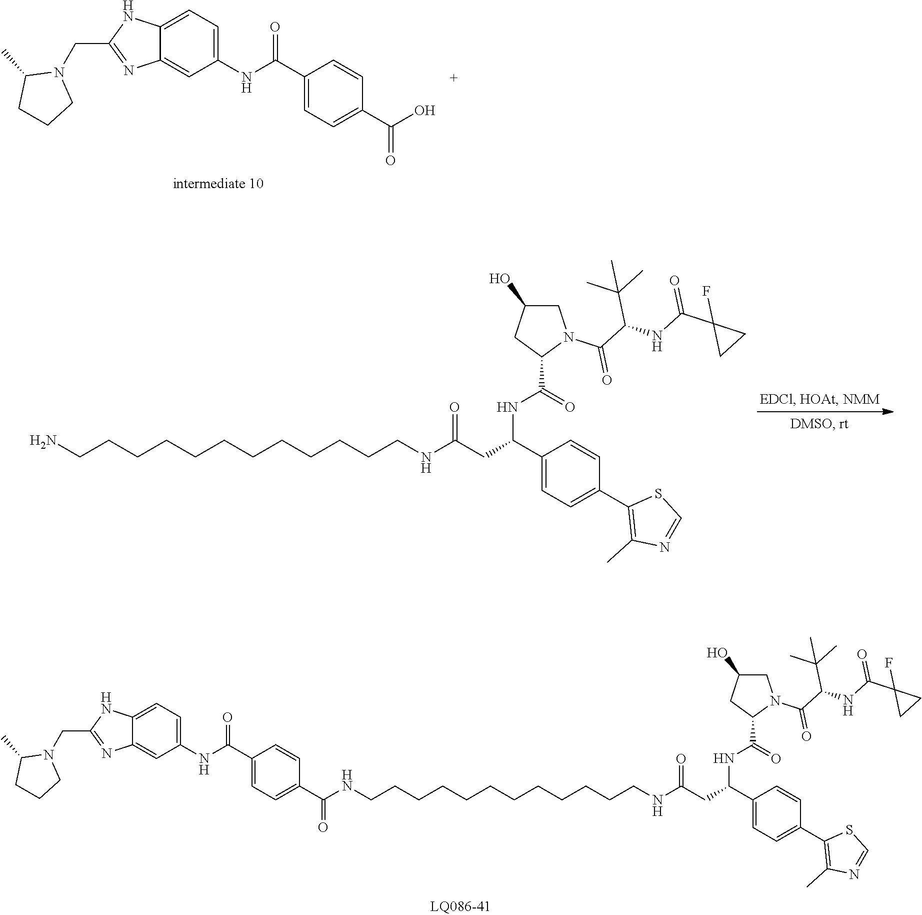 [SMILES: C[C@H]1CCCN1Cc1nc2cc(NC(=O)c3ccc(C(=O)O)cc3)ccc2[nH]1 ; Cc1ncsc1-c1ccc([C@H](CC(=O)NCCCCCCCCCCCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCCCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1]

LQ086-41 was synthesized following the standard procedure for preparing LQ076-105 from intermediate 10 (10 mg, 0.02 mmol), (2S,4R)—N—((S)-3-((12-aminododecyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (17.4 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ086-41 was obtained as white solid in TFA salt form (19.4 mg, 72%). ¹H NMR (600 MHz, Methanol-d₄) δ 8.84 (s, 1H), 8.18 (d, J=2.0 Hz, 1H), 7.94 (d, J=8.5 Hz, 2H), 7.85 (d, J=8.4 Hz, 2H), 7.56 (d, J=8.8 Hz, 1H), 7.44 (dd, J=8.7, 2.0 Hz, 1H), 7.40-7.32 (m, 4H), 5.21 (dd, J=8.5, 5.8 Hz, 1H), 4.69 (d, J=14.6 Hz, 1H), 4.64 (d, J=9.5 Hz, 1H), 4.50-4.42 (m, 2H), 4.36-4.33 (m, 1H), 3.75-3.71 (m, 1H), 3.69-3.59 (m, 3H), 3.39-3.33 (m, 1H), 3.31 (t, J=7.2 Hz, 2H), 3.04-2.98 (m, 1H), 2.96-2.89 (m, 1H), 2.74 (dd, J=14.1, 5.9 Hz, 1H), 2.64 (dd, J=14.0, 8.5 Hz, 1H), 2.38 (s, 3H), 2.32-2.24 (m, 1H), 2.13-2.07 (m, 1H), 2.06-1.95 (m, 2H), 1.89-1.83 (m, 1H), 1.75-1.67 (m, 1H), 1.54 (p, J=7.2 Hz, 2H), 1.39 (d, J=6.5 Hz, 3H), 1.34-1.06 (m, 20H), 1.05-0.99 (m, 2H), 0.96 (s, 9H). HRMS m/z [M+H]⁺ calcd for C₆₁H₈₂FN₁₀O₇S⁺ 1117.6017, found 1117.6005.

### Example 154

### Synthesis of LQ086-76 and LQ086-76Na

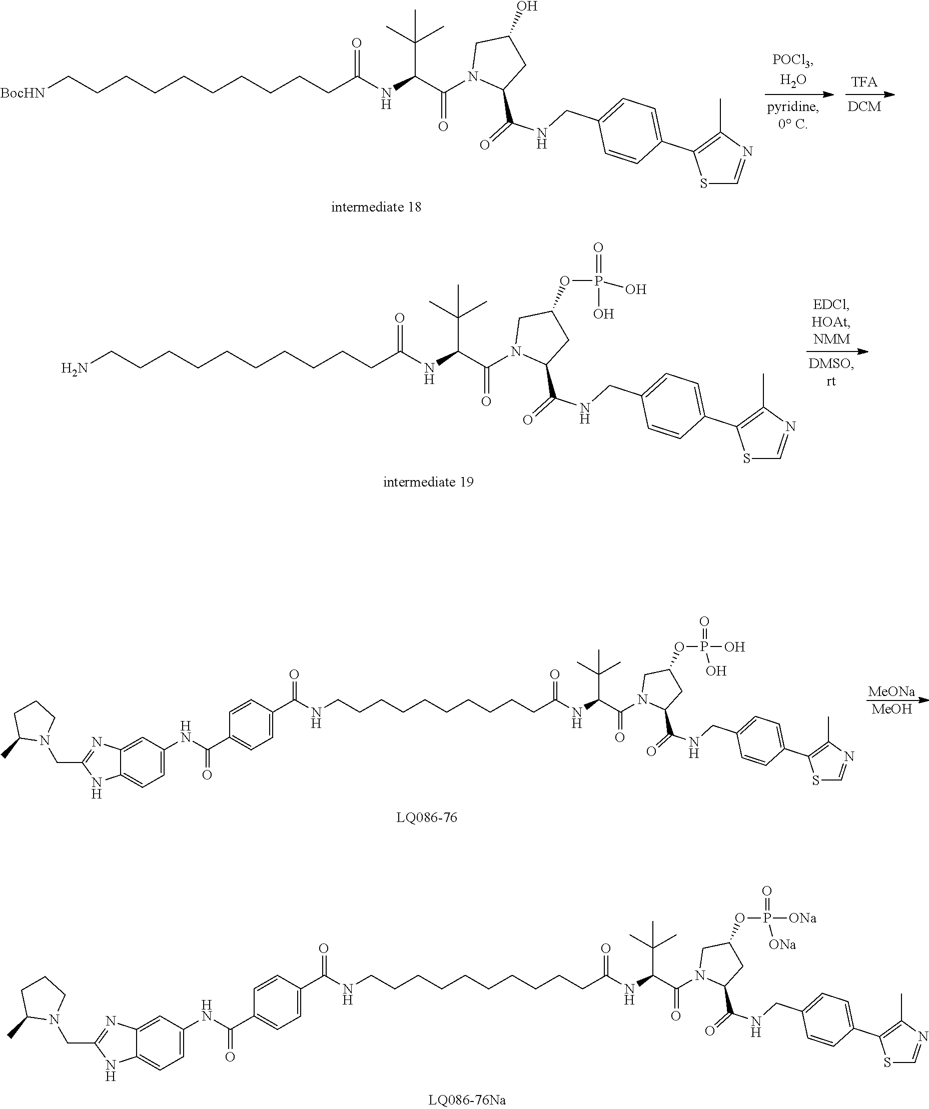

### Intermediate 19: (3R,5S)-1-((S)-2-(11-aminoundecanamido)-3,3-dimethylbutanoyl)-5-((4-(4-methylthiazol-5-yl)benzyl)carbamoyl)pyrrolidin-3-yl dihydrogen phosphate

An ice bath cooled solution of POCl₃ (40 μL, 0.35 mmol) in 0.5 mL dry pyridine was slowly added to a cooled solution of intermediate 18 (100 mg, 0.14 mmol) in 1 mL dry pyridine. The reaction mixture was keeping stirred at ice bath until intermediate 18 was disappeared. Then water was added. After being stirred for 10 mins, the reaction mixture was purified by reverse phase C18 column (10%-100% methanol/0.1% TFA in water) to afford a coler less oil. The obtained oil was dissolved in 0.5 mL DCM, to the resulting solution was added 0.3 mL TFA. After being stirred for 1 h at room temperature, the reaction mixture was concentrated and the residue was purified by reverse phase C18 column (10%-100% methanol/0.1% TFA in water) to afford intermediate 19 as white solid in TFA salt form (78 mg, 69%). ¹H NMR (600 MHz, Methanol-d₄) δ 9.11 (s, 1H), 7.51-7.48 (m, 2H), 7.46-7.43 (m, 2H), 4.63-4.54 (m, 3H), 4.38 (d, J=15.5 Hz, 1H), 4.26-4.20 (m, 1H), 3.95-3.90 (m, 1H), 2.92 (t, J=7.7 Hz, 2H), 2.58-2.50 (m, 4H), 2.37-2.30 (m, 1H), 2.29-2.20 (m, 2H), 1.70-1.57 (m, 4H), 1.44-1.31 (m, 13H), 1.06 (s, 9H). MS (ESI): m/z 694.4 [M+H]⁺.

### LQ086-76

LQ086-76 was synthesized following the similar procedure for preparing LQ076-105 from intermediate 10 (10 mg, 0.02 mmol), intermediate 19 (16.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ086-76 was obtained as white solid in free base (16.2 mg, 77%).

¹H NMR (600 MHz, Methanol-d₄) δ 8.88 (s, 1H), 8.21 (s, 1H), 8.09-8.02 (m, 2H), 7.97-7.91 (m, 2H), 7.66 (d, J=8.8 Hz, 1H), 7.61 (dd, J=8.7, 1.9 Hz, 1H), 7.48-7.42 (m, 2H), 7.41-7.38 (m, 2H), 4.99-4.93 (m, 1H), 4.77 (d, J=14.5 Hz, 1H), 4.63-4.57 (m, 2H), 4.56-4.49 (m, 2H), 4.35 (dd, J=15.5, 4.8 Hz, 1H), 4.19-4.13 (m, 1H), 3.90-3.86 (m, 1H), 3.74-3.62 (m, 2H), 3.46-3.35 (m, 4H), 2.55-2.49 (m, 1H), 2.42-2.33 (m, 1H), 2.31-2.22 (m, 1H), 2.21-2.06 (m, 3H), 1.88-1.79 (m, 1H), 1.67-1.60 (m, 2H), 1.59-1.46 (m, 5H), 1.43-1.23 (m, 12H), 1.03 (s, 9H). HRMS m/z [M+H]⁺ calcd for C₅₄H₇₃N₉O₉PS⁺ 1054.4984, found 1054.4997.

### LQ086-76Na

LQ076-76 (42 mg, 0.039 mmol) was dissolved in methanol. After the reaction mixture was cooled to ice bath, two equivalent of MeONa (0.5 M in methanol) was added, the mixture was stirred at RT for 1 h. Then evaporated the solvent to give the desired product as white solid. ¹H NMR (600 MHz, Methanol-d₄) δ 8.76 (s, 1H), 7.98 (d, J=2.0 Hz, 1H), 7.94 (d, J=8.4 Hz, 2H), 7.85 (d, J=8.4 Hz, 1H), 7.43 (d, J=8.6 Hz, 1H), 7.38-7.33 (m, 3H), 7.32-7.28 (m, 2H), 4.53-4.50 (m, 1H), 4.50-4.44 (m, 1H), 4.42 (d, J=15.5 Hz, 1H), 4.26 (d, J=15.4 Hz, 1H), 4.06 (d, J=14.3 Hz, 1H), 4.02 (d, J=11.1 Hz, 1H), 3.74-3.69 (m, 1H), 3.55 (d, J=14.3 Hz, 1H), 3.30 (t, J=7.1 Hz, 2H), 3.07-2.99 (m, 3H), 2.99-2.92 (m, 1H), 2.54-2.46 (m, 1H), 2.37 (s, 3H), 2.34-2.26 (m, 2H), 2.21-2.15 (m, 1H), 2.15-2.09 (m, 1H), 2.00-1.88 (m, 1H), 1.73-1.62 (m, 1H), 1.60-1.45 (m, 4H), 1.44-1.34 (m, 1H), 1.35-1.18 (m, 8H), 1.09 (d, J=6.1 Hz, 3H), 0.93 (s, 9H). HRMS m/z [M+H]⁺ calcd for C₅₄H₇₃N₉O₉PS⁺ 1054.4984, found 1054.5027.

### Synthesis of LQ108-4

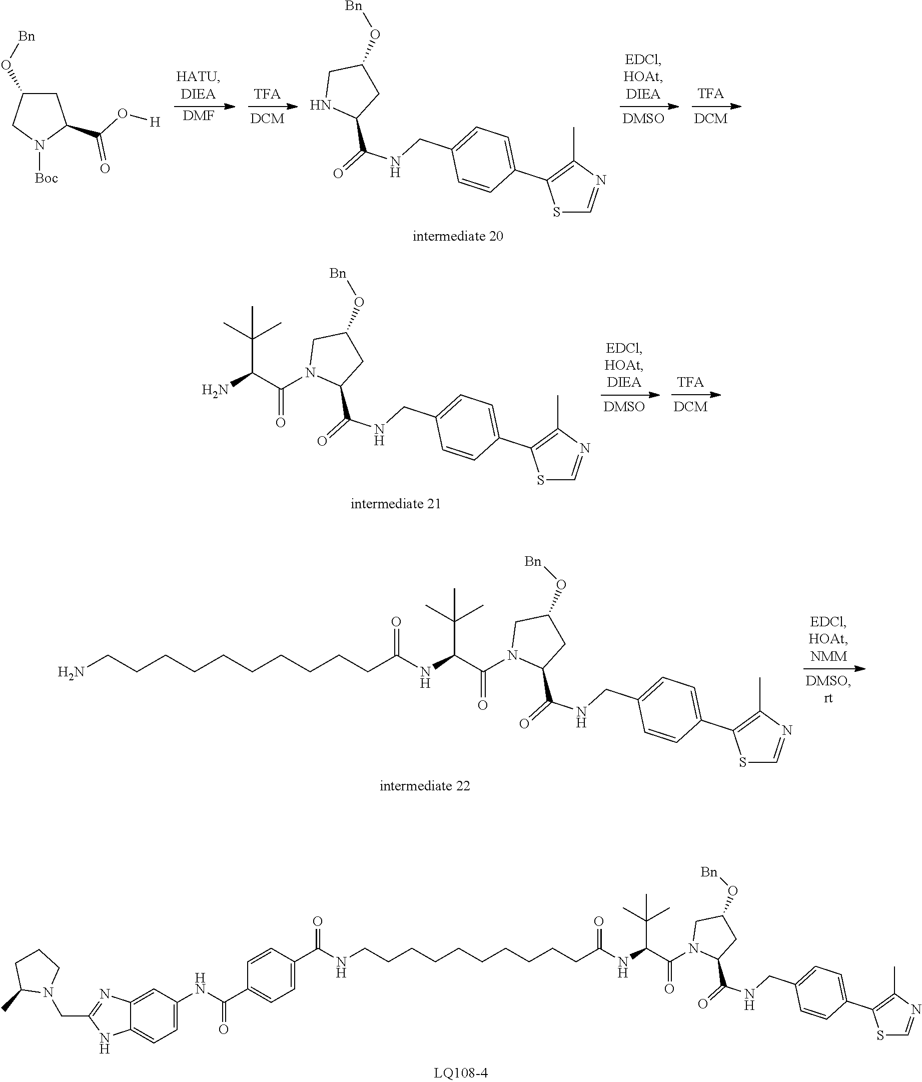

### Intermediate 20 (2S,4R)-4-(benzyloxy)-N-(4-(4-methylthiazol-5-yl)benzyl)pyrrolidine-2-carboxamide

A solution of (4-(4-methylthiazol-5-yl)phenyl)methanamine (500 mg, 1.55 mmol) was dissolved in DMF and treated with (2S,4R)-4-(benzyloxy)-1-[(tert-butoxy)carbonyl]pyrrolidine-2-carboxylic acid (500 mg, 1.55 mmol), HATU (707 mg, 1.8 mmol) and DIEA (845 μL, 4.8 mmol). After being stirred 1 h at room temperature, the reaction mixture was poured into ice water, aqueous phase was extracted with ethyl acetate. The combined organic phase was washed with brine twice, dried and concentrated. The resulting residue was purified by silica gel flash chromatography to give the compound as yellow solid. The obtained solid was dissolved in 5 mL DCM, to the resulting solution was added 3 mL TFA. After being stirred for 1 h at room temperature, the reaction mixture was concentrated and the residue was purified by reverse phase C18 column (10%-100% methanol/0.1% TFA in water) to afford intermediate 20 as white solid in TFA salt form (500 mg, 79% yield for 2 steps). ¹H NMR (600 MHz, Methanol-d₄) δ 8.93 (s, 1H), 7.46-7.43 (m, 2H), 7.42-7.39 (m, 2H), 7.36-7.31 (m, 4H), 7.29-7.25 (m, 1H), 4.56 (d, J=3.4 Hz, 2H), 4.51-4.44 (m, 3H), 4.43-4.40 (m, 1H), 3.56-3.52 (m, 1H), 3.46 (dd, J=12.6, 3.9 Hz, 1H), 2.74-2.68 (m, 1H), 2.46 (s, 3H), 2.10-2.04 (m, 1H). MS (ESI): m/z 408.3 [M+H]⁺.

### Intermediate 21 (2S,4R)-1-((S)-2-amino-3,3-dimethylbutanoyl)-4-(benzyloxy)-N-(4-(4-methylthiazol-5-yl)benzyl)pyrrolidine-2-carboxamide

Intermediate 21 was synthesized according to the procedures for the preparation of intermediate 20 as a white solid in 79% yield. ¹H NMR (600 MHz, Methanol-d₄) δ 9.07 (s, 1H), 7.51-7.48 (m, 2H), 7.45-7.42 (m, 2H), 7.39-7.33 (m, 4H), 7.32-7.28 (m, 1H), 4.68 (dd, J=9.7, 7.5 Hz, 1H), 4.61-4.55 (m, 3H), 4.41-4.34 (m, 2H), 4.13 (s, 1H), 4.11-4.06 (m, 1H), 3.75 (dd, J=11.5, 3.7 Hz, 1H), 2.59-2.54 (m, 1H), 2.50 (s, 3H), 2.13-2.07 (m, 1H), 1.16 (s, 9H). MS (ESI): m/z 521.3 [M+H]⁺.

### Intermediate 22 (2S,4R)-1-((S)-2-(11-aminoundecanamido)-3,3-dimethylbutanoyl)-4-(benzyloxy)-N-(4-(4-methylthiazol-5-yl)benzyl)pyrrolidine-2-carboxamide

Intermediate 22 was synthesized according to the procedures for the preparation of intermediate 20 as a white solid in 83% yield. MS (ESI): m/z 704.4 [M+H]⁺.

LQ108-4 was synthesized following the standard procedure for preparing LQ076-105 from intermediate 10 (10 mg, 0.02 mmol), intermediate 22 (16.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-4 was obtained as white solid in TFA salt form (17.9 mg, 69%). ¹H NMR (600 MHz, Methanol-d₄) δ 9.16 (s, 1H), 8.36 (d, J=1.9 Hz, 1H), 8.05 (d, J=8.5 Hz, 2H), 7.96 (d, J=8.4 Hz, 2H), 7.72 (d, J=8.8 Hz, 1H), 7.63 (dd, J=8.8, 2.0 Hz, 1H), 7.51-7.42 (m, 4H), 7.35-7.29 (m, 4H), 7.29-7.24 (m, 1H), 4.89 (d, J=14.7 Hz, 1H), 4.74-4.71 (m, 1H), 4.67-4.48 (m, 5H), 4.38 (d, J=15.5 Hz, 1H), 4.32-4.27 (m, 2H), 3.80-3.70 (m, 3H), 3.49-3.44 (m, 1H), 3.41 (t, J=7.2 Hz, 2H), 2.51 (s, 3H), 2.44-2.37 (m, 2H), 2.33-2.20 (m, 2H), 2.19-2.07 (m, 3H), 1.87-1.80 (m, 1H), 1.67-1.55 (m, 4H), 1.51 (d, J=6.5 Hz, 3H), 1.43-1.26 (m, 12H), 1.05 (s, 9H). HRMS m/z [M+H]⁺ calcd for C₆₁H₇₈N₉O₆S⁺ 1064.5790, found 1064.5825.

### Synthesis of LQ108-5

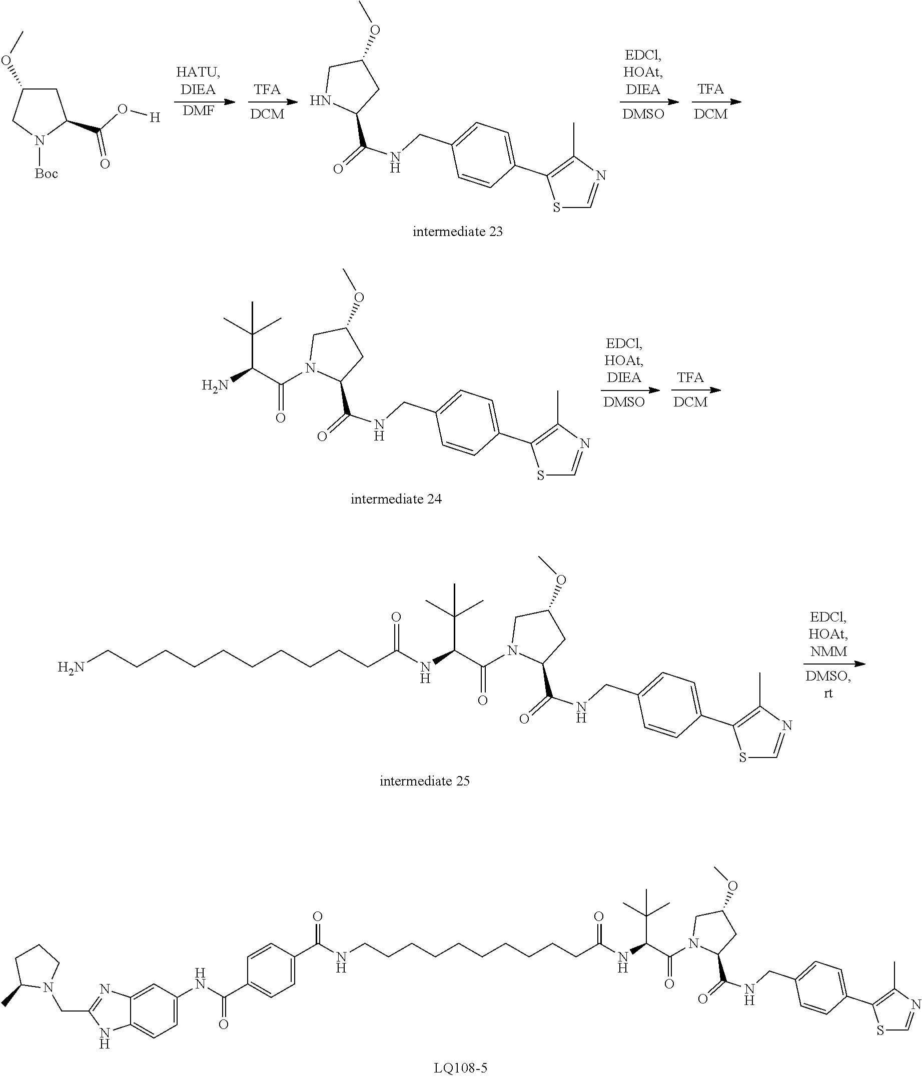

### Intermediate 23 (2S,4R)-4-methoxy-N-(4-(4-methylthiazol-5-yl)benzyl)pyrrolidine-2-carboxamide

Intermediate 23 was synthesized according to the procedures for the preparation of intermediate 20 as a white solid in 65% yield. ¹H NMR (600 MHz, Methanol-d₄) δ 9.12 (s, 1H), 7.50-7.43 (m, 4H), 4.52 (d, J=2.3 Hz, 2H), 4.46 (dd, J=10.8, 7.4 Hz, 1H), 4.23 (t, J=4.0 Hz, 1H), 3.55-3.51 (m, 1H), 3.46 (dd, J=12.6, 3.8 Hz, 1H), 3.37 (s, 3H), 2.73-2.67 (m, 1H), 2.51 (s, 3H), 2.10-2.03 (m, 1H). MS (ESI): m/z 331.2 [M+H]⁺.

### Intermediate 24 (2S,4R)-1-((S)-2-amino-3,3-dimethylbutanoyl)-4-methoxy-N-(4-(4-methylthiazol-5-yl)benzyl)pyrrolidine-2-carboxamide

Intermediate 24 was synthesized according to the procedures for the preparation of intermediate 20 as a white solid in 83% yield. ¹H NMR (600 MHz, Methanol-d₄) δ 9.03 (s, 1H), 7.51-7.48 (m, 2H), 7.46-7.42 (m, 2H), 4.63-4.55 (m, 2H), 4.38 (d, J=15.5 Hz, 1H), 4.16-4.11 (m, 2H), 4.07-4.02 (m, 1H), 3.69 (dd, J=11.6, 3.6 Hz, 1H), 3.37 (s, 3H), 2.52-2.45 (m, 4H), 2.10-2.03 (m, 1H), 1.16 (s, 9H). MS (ESI): m/z 445.3 [M+H]⁺.

### Intermediate 25 (2S,4R)-1-((S)-2-(11-aminoundecanamido)-3,3-dimethylbutanoyl)-4-methoxy-N-(4-(4-methylthiazol-5-yl)benzyl)pyrrolidine-2-carboxamide

Intermediate 25 was synthesized according to the procedures for the preparation of intermediate 20 as a white solid in 77% yield. ¹H NMR (600 MHz, Methanol-d₄) δ 8.91 (s, 1H), 7.44-7.36 (m, 4H), 4.61 (s, 1H), 4.49 (d, J=15.5 Hz, 1H), 4.42 (dd, J=9.4, 7.5 Hz, 1H), 4.32 (d, J=15.4 Hz, 1H), 4.13-4.09 (m, 1H), 4.06-4.03 (m, 1H), 3.66 (dd, J=11.3, 3.7 Hz, 1H), 3.28 (s, 3H), 2.86 (t, J=7.7 Hz, 2H), 2.44 (s, 3H), 2.34-2.16 (m, 3H), 2.04-1.99 (m, 1H), 1.63-1.52 (m, 5H), 1.39-1.25 (m, 12H), 0.99 (s, 9H). MS (ESI): m/z 628.8 [M+H]⁺.

LQ108-5 was synthesized following the standard procedure for preparing LQ076-105 from intermediate 10 (10 mg, 0.02 mmol), intermediate 25 (14.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-5 was obtained as white solid in TFA salt form (17.7 mg, 73%). ¹H NMR (600 MHz, Methanol-d₄) δ 9.12 (s, 1H), 8.35 (d, J=1.9 Hz, 1H), 8.05 (d, J=8.4 Hz, 2H), 7.96 (d, J=8.5 Hz, 2H), 7.71 (d, J=8.8 Hz, 1H), 7.62 (dd, J=8.8, 1.9 Hz, 1H), 7.50-7.46 (m, 2H), 7.45-7.41 (m, 2H), 4.88 (d, J=14.6 Hz, 1H), 4.69-4.62 (m, 2H), 4.54 (d, J=15.5 Hz, 1H), 4.50 (dd, J=9.4, 7.5 Hz, 1H), 4.38 (d, J=15.5 Hz, 1H), 4.18 (d, J=11.6 Hz, 1H), 4.12-4.07 (m, 1H), 3.80-3.68 (m, 3H), 3.50-3.39 (m, 3H), 2.50 (s, 3H), 2.44-2.22 (m, 4H), 2.19-2.04 (m, 3H), 1.88-1.79 (m, 1H), 1.68-1.57 (m, 5H), 1.51 (d, J=6.5 Hz, 3H), 1.44-1.29 (m, 10H), 1.05 (s, 9H). HRMS m/z [M+H]⁺ calcd for C₅₅H₇₄N₉O₆S⁺ 988.5477, found 988.5576.

### Example 157

### Synthesis of LQ108-6

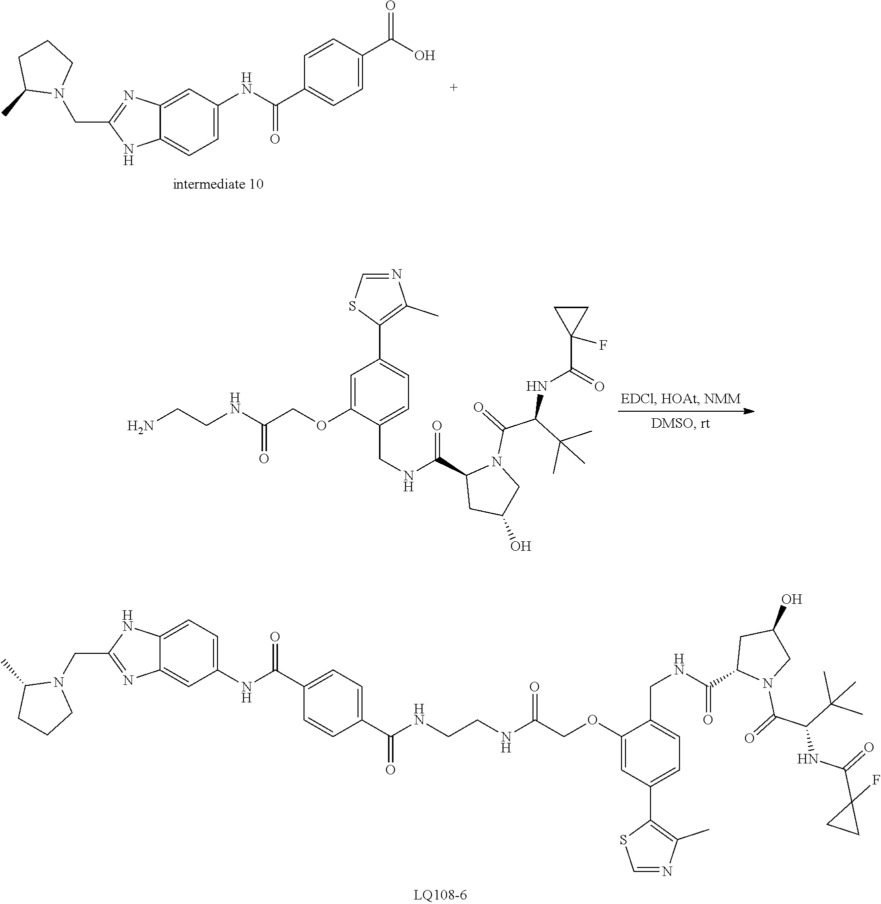 [SMILES: C[C@H]1CCCN1Cc1nc2cc(NC(=O)c3ccc(C(=O)O)cc3)ccc2[nH]1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCNC(=O)c2ccc(C(=O)Nc3ccc4[nH]c(CN5CCC[C@@H]5C)nc4c3)cc2)c1]

LQ108-6 was synthesized following the standard procedure for preparing LQ076-105 from intermediate 10 (10 mg, 0.02 mmol), (2S,4R)—N-(2-(2-((2-aminoethyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (14.1 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-6 was obtained as white solid in TFA salt form (18.2 mg, 75%). ¹H NMR (600 MHz, Methanol-d₄) δ 9.10 (s, 1H), 8.35 (d, J=2.0 Hz, 1H), 8.06-8.01 (m, 2H), 7.95-7.90 (m, 2H), 7.71 (d, J=8.8 Hz, 1H), 7.61 (dd, J=8.8, 2.0 Hz, 1H), 7.52-7.45 (m, 2H), 7.09 (dd, J=7.8, 1.6 Hz, 1H), 7.01 (d, J=1.7 Hz, 1H), 4.87 (d, J=14.7 Hz, 1H), 4.75-4.72 (m, 1H), 4.70-4.58 (m, 5H), 4.52-4.46 (m, 2H), 3.89-3.72 (m, 4H), 3.66-3.56 (m, 4H), 3.51-3.43 (m, 1H), 2.50 (s, 3H), 2.45-2.37 (m, 1H), 2.24-2.05 (m, 4H), 1.88-1.80 (m, 1H), 1.52 (d, J=6.5 Hz, 3H), 1.39-1.24 (m, 4H), 1.01 (s, 9H). HRMS m/z [M+H]⁺ calcd for C₅₁H₆₂FN₁₀O₈S⁺ 993.4451, found 933.4523.

### Example 158

### Synthesis of LQ108-7

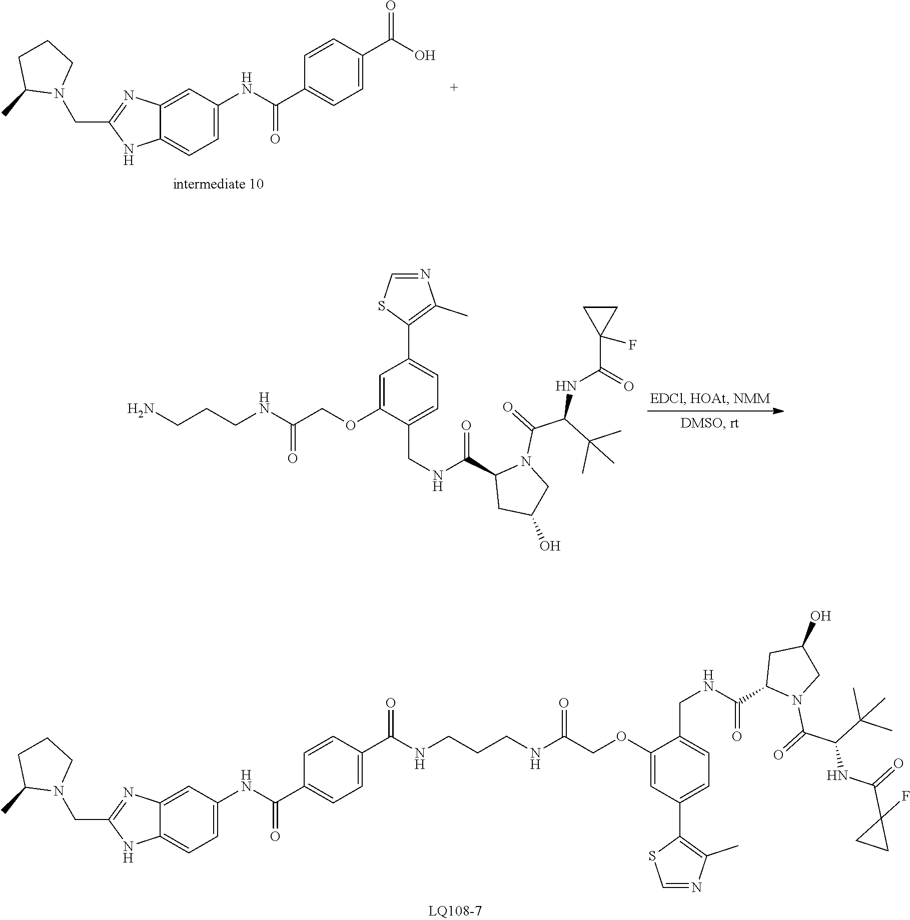 [SMILES: C[C@H]1CCCN1Cc1nc2cc(NC(=O)c3ccc(C(=O)O)cc3)ccc2[nH]1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCNC(=O)c2ccc(C(=O)Nc3ccc4[nH]c(CN5CCC[C@@H]5C)nc4c3)cc2)c1]

LQ108-7 was synthesized following the standard procedure for preparing LQ076-105 from intermediate 10 (10 mg, 0.02 mmol (2S,4R)—N-(2-(2-((3-aminopropyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (14.4 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-7 was obtained as white solid in TFA salt form (17.6 mg, 71%). ¹H NMR (600 MHz, Methanol-d₄) δ 9.04 (s, 1H), 8.32 (d, J=1.9 Hz, 1H), 8.05-8.02 (m, 2H), 7.99-7.96 (m, 2H), 7.70 (d, J=8.8 Hz, 1H), 7.59 (dd, J=8.8, 2.0 Hz, 1H), 7.53 (d, J=7.7 Hz, 1H), 7.48 (dd, J=9.4, 3.3 Hz, 1H), 7.12 (dd, J=7.7, 1.6 Hz, 1H), 7.01 (d, J=1.6 Hz, 1H), 4.85 (d, J=14.6 Hz, 1H), 4.75-4.71 (m, 1H), 4.67-4.64 (m, 2H), 4.64-4.58 (m, 3H), 4.55-4.48 (m, 2H), 3.88-3.82 (m, 1H), 3.81-3.71 (m, 3H), 3.51-3.41 (m, 6H), 2.51 (s, 3H), 2.44-2.37 (m, 1H), 2.23-2.06 (m, 3H), 1.93-1.87 (m, 2H), 1.86-1.80 (m, 1H), 1.52 (d, J=6.5 Hz, 3H), 1.40-1.23 (m, 4H), 1.01 (s, 9H). HRMS m/z [M+H]⁺ calcd for C₅₂H₆₄FN₁₀O₈S⁺ 1007.4608, found 1007.4653.

### Example 159

### Synthesis of LQ108-8

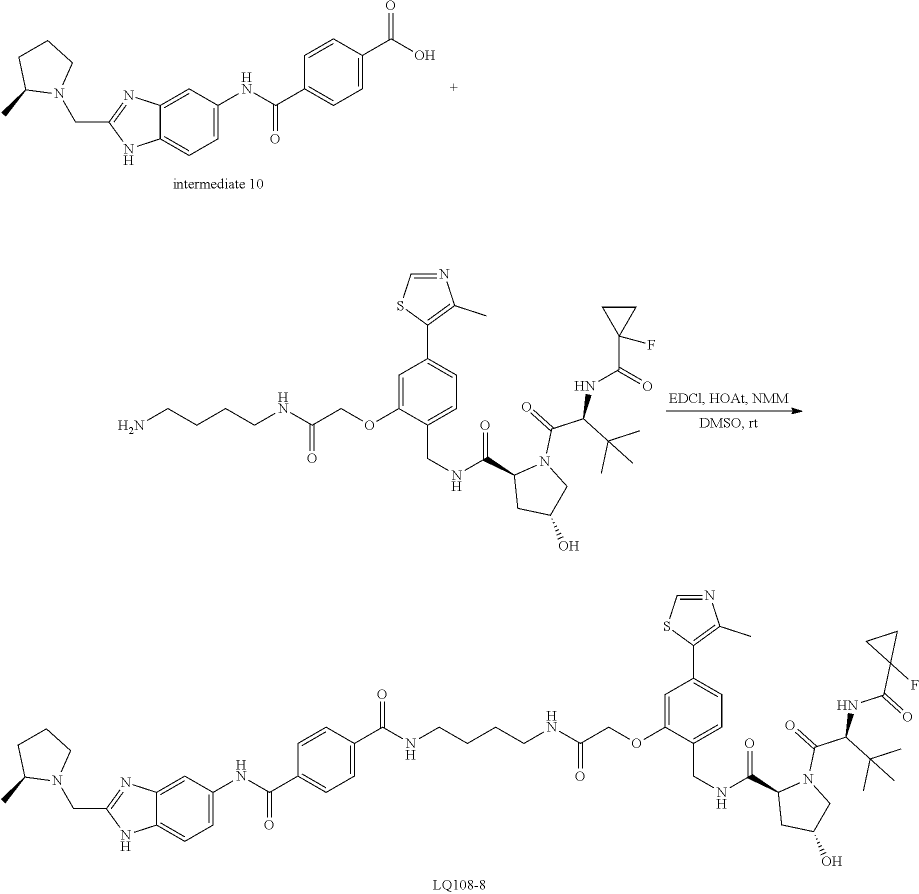 [SMILES: C[C@H]1CCCN1Cc1nc2cc(NC(=O)c3ccc(C(=O)O)cc3)ccc2[nH]1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCNC(=O)c2ccc(C(=O)Nc3ccc4[nH]c(CN5CCC[C@@H]5C)nc4c3)cc2)c1]

LQ108-8 was synthesized following the standard procedure for preparing LQ076-105 from intermediate 10 (10 mg, 0.02 mmol), (2S,4R)—N-(2-(2-((4-aminobutyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (14.7 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-8 was obtained as white solid in TFA salt form (18.2 mg, 75%). ¹H NMR (600 MHz, Methanol-d₄) δ 9.06 (s, 1H), 8.33 (d, J=1.9 Hz, 1H), 8.07-8.02 (m, 2H), 7.99-7.94 (m, 2H), 7.70 (d, J=8.8 Hz, 1H), 7.59 (dd, J=8.8, 2.0 Hz, 1H), 7.51 (d, J=7.8 Hz, 1H), 7.48 (dd, J=9.4, 3.3 Hz, 1H), 7.11 (dd, J=7.7, 1.6 Hz, 1H), 7.00 (d, J=1.6 Hz, 1H), 4.85 (d, J=14.7 Hz, 1H), 4.76-4.72 (m, 1H), 4.65-4.57 (m, 5H), 4.51-4.45 (m, 2H), 3.86-3.71 (m, 4H), 3.50-3.35 (m, 6H), 2.51 (s, 3H), 2.44-2.37 (m, 1H), 2.24-2.05 (m, 3H), 1.87-1.80 (m, 1H), 1.71-1.64 (m, 4H), 1.52 (d, J=6.5 Hz, 3H), 1.40-1.24 (m, 4H), 1.02 (s, 9H). HRMS m/z [M+H]⁺ calcd for C₅₃H₆₆FN₁₀O₈S⁺ 1021.4764, found 1021.4816.

### Example 160

### Synthesis of LQ108-9

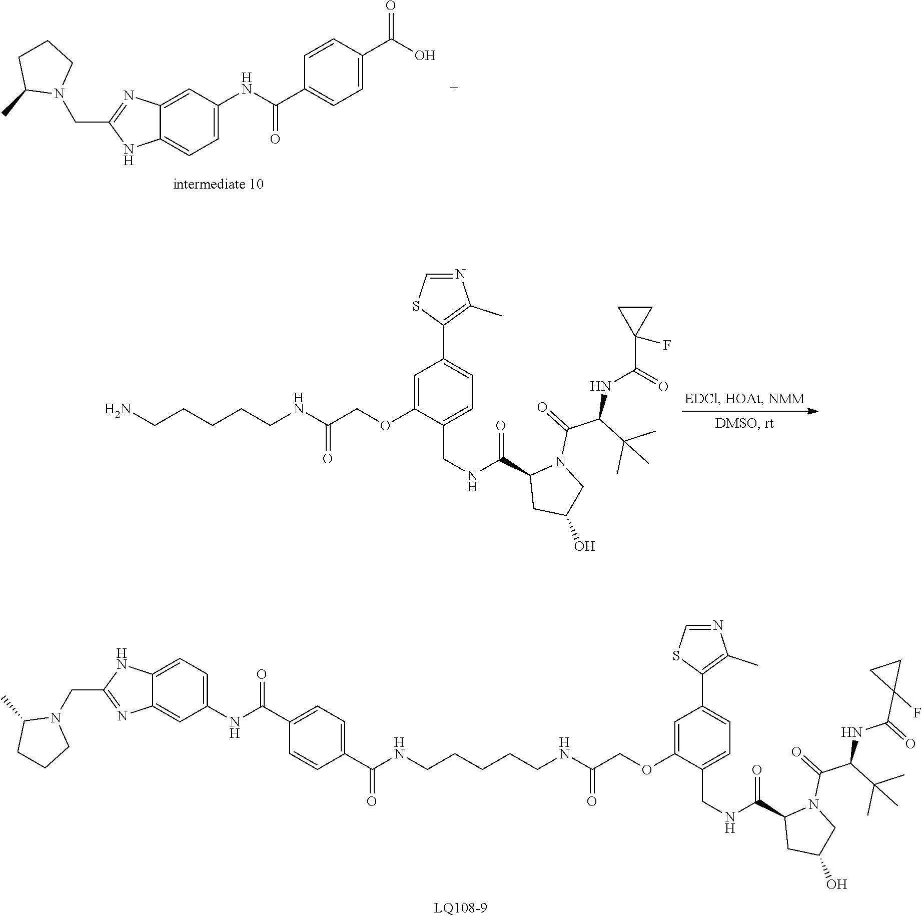 [SMILES: C[C@H]1CCCN1Cc1nc2cc(NC(=O)c3ccc(C(=O)O)cc3)ccc2[nH]1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCNC(=O)c2ccc(C(=O)Nc3ccc4[nH]c(CN5CCC[C@@H]5C)nc4c3)cc2)c1]

LQ108-9 was synthesized following the standard procedure for preparing LQ076-105 from intermediate 10 (10 mg, 0.02 mmol), (2S,4R)—N-(2-(2-((5-aminopentyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (14.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-9 was obtained as white solid in TFA salt form (20 mg, 79%). ¹H NMR (600 MHz, Methanol-d₄) δ 9.02 (s, 1H), 8.31 (d, J=2.0 Hz, 1H), 8.05-8.00 (m, 2H), 7.98-7.93 (m, 2H), 7.69 (d, J=8.7 Hz, 1H), 7.57 (dd, J=8.7, 2.0 Hz, 1H), 7.51-7.46 (m, 2H), 7.11 (dd, J=7.7, 1.6 Hz, 1H), 6.99 (d, J=1.6 Hz, 1H), 4.83 (d, J=14.7 Hz, 1H), 4.76-4.72 (m, 1H), 4.62-4.57 (m, 5H), 4.52-4.48 (m, 1H), 4.44 (d, J=15.0 Hz, 1H), 3.88-3.83 (m, 1H), 3.82-3.72 (m, 3H), 3.50-3.44 (m, 1H), 3.43-3.34 (m, 5H), 2.51 (s, 3H), 2.44-2.37 (m, 1H), 2.25-2.05 (m, 3H), 1.87-1.79 (m, 1H), 1.71-1.62 (m, 4H), 1.52 (d, J=6.5 Hz, 3H), 1.47-1.25 (m, 6H), 1.02 (s, 9H). HRMS m/z [M+H]⁺ calcd for C₅₄H₆₈FN₁₀O₈S⁺ 1035.4921, found 1035.4963.

### Example 161

### Synthesis of LQ108-10

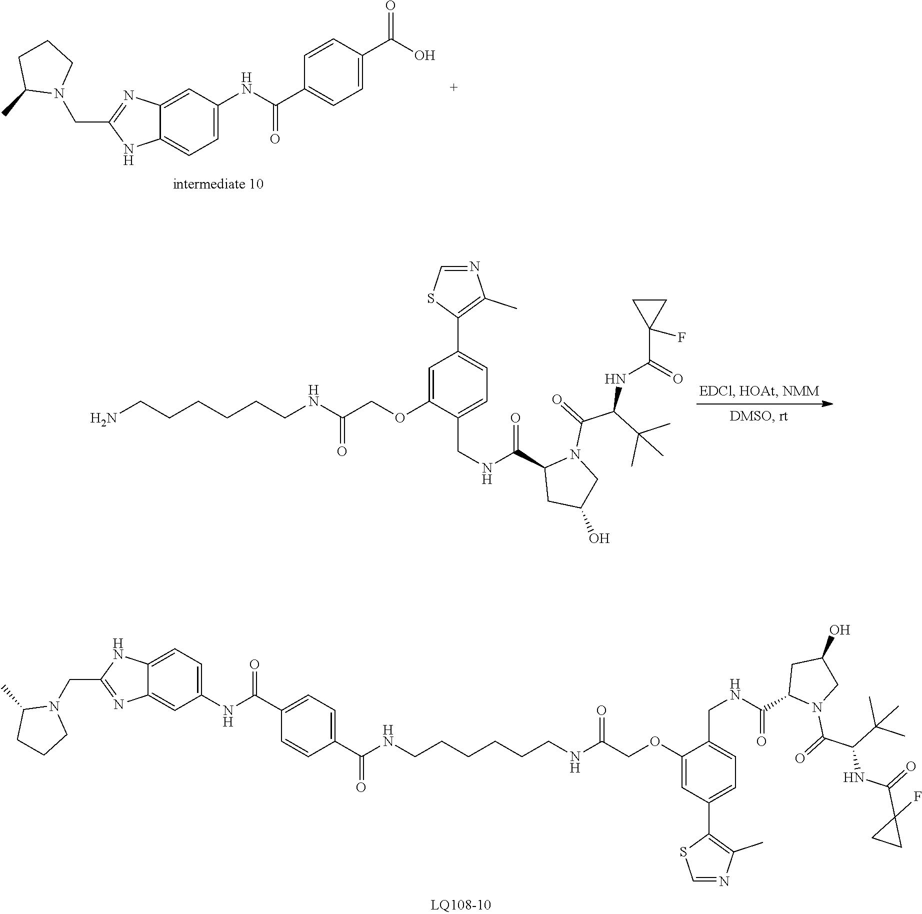 [SMILES: C[C@H]1CCCN1Cc1nc2cc(NC(=O)c3ccc(C(=O)O)cc3)ccc2[nH]1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCNC(=O)c2ccc(C(=O)Nc3ccc4[nH]c(CN5CCC[C@@H]5C)nc4c3)cc2)c1]

LQ108-10 was synthesized following the standard procedure for preparing LQ076-105 from intermediate 10 (10 mg, 0.02 mmol), (2S,4R)—N-(2-(2-((6-aminohexyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (15.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-10 was obtained as white solid in TFA salt form (18.9 mg, 74%). ¹H NMR (600 MHz, Methanol-d₄) δ 9.08 (s, 1H), 8.34 (d, J=1.9 Hz, 1H), 8.07-8.02 (m, 2H), 7.99-7.94 (m, 2H), 7.71 (d, J=8.8 Hz, 1H), 7.60 (dd, J=8.8, 2.0 Hz, 1H), 7.50 (dd, J=21.8, 7.0 Hz, 2H), 7.12 (dd, J=7.7, 1.6 Hz, 1H), 7.00 (d, J=1.6 Hz, 1H), 4.86 (d, J=14.6 Hz, 1H), 4.76-4.72 (m, 1H), 4.65-4.58 (m, 5H), 4.52-4.45 (m, 2H), 3.88-3.83 (m, 1H), 3.81-3.71 (m, 3H), 3.50-3.44 (m, 1H), 3.40 (t, J=7.1 Hz, 2H), 3.32-3.28 (m, 1H), 2.52 (s, 3H), 2.45-2.36 (m, 1H), 2.25-2.05 (m, 4H), 1.88-1.80 (m, 1H), 1.66-1.57 (m, 4H), 1.52 (d, J=6.5 Hz, 3H), 1.45-1.24 (m, 9H), 1.02 (s, 9H). HRMS m/z [M+H]⁺ calcd for C₅₅H₇₀FN₁₀O₈S⁺ 1049.5077, found 1049.5140.

### Example 162

### Synthesis of LQ108-11

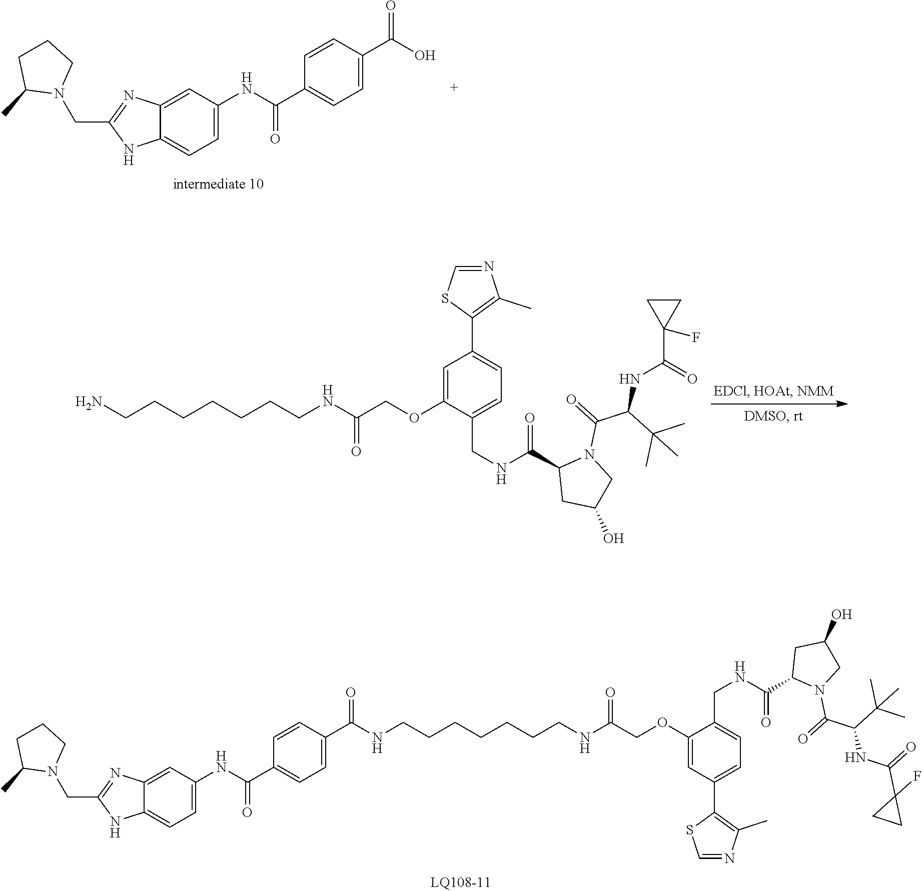 [SMILES: C[C@H]1CCCN1Cc1nc2cc(NC(=O)c3ccc(C(=O)O)cc3)ccc2[nH]1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCCNC(=O)c2ccc(C(=O)Nc3ccc4[nH]c(CN5CCC[C@@H]5C)nc4c3)cc2)c1]

LQ108-11 was synthesized following the standard procedure for preparing LQ076-105 from intermediate 10 (10 mg, 0.02 mmol), (2S,4R)—N-(2-(2-((7-aminoheptyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (15.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-11 was obtained as white solid in TFA salt form (18.3 mg, 70%). ¹H NMR (600 MHz, Methanol-d₄) δ 9.04 (s, 1H), 8.32 (d, J=1.9 Hz, 1H), 8.07-8.02 (m, 2H), 7.99-7.94 (m, 2H), 7.69 (d, J=8.8 Hz, 1H), 7.58 (dd, J=8.8, 2.0 Hz, 1H), 7.52-7.46 (m, 2H), 7.11 (dd, J=7.7, 1.6 Hz, 1H), 6.99 (d, J=1.6 Hz, 1H), 4.84 (d, J=14.6 Hz, 1H), 4.77-4.72 (m, 1H), 4.65-4.57 (m, 5H), 4.52-4.45 (m, 2H), 3.89-3.83 (m, 1H), 3.82-3.72 (m, 3H), 3.51-3.43 (m, 1H), 3.41 (t, J=7.2 Hz, 2H), 3.32-3.27 (m, 3H), 2.51 (s, 3H), 2.45-2.36 (m, 1H), 2.25-2.06 (m, 3H), 1.87-1.79 (m, 1H), 1.67-1.55 (m, 4H), 1.52 (d, J=6.5 Hz, 3H), 1.42-1.25 (m, 10H), 1.03 (s, 9H). HRMS m/z [M+H]⁺ calcd for C₅₆H₇₂FN₁₀O₈S⁺ 1063.5234, found 1063.5276.

### Example 163

### Synthesis of LQ108-12

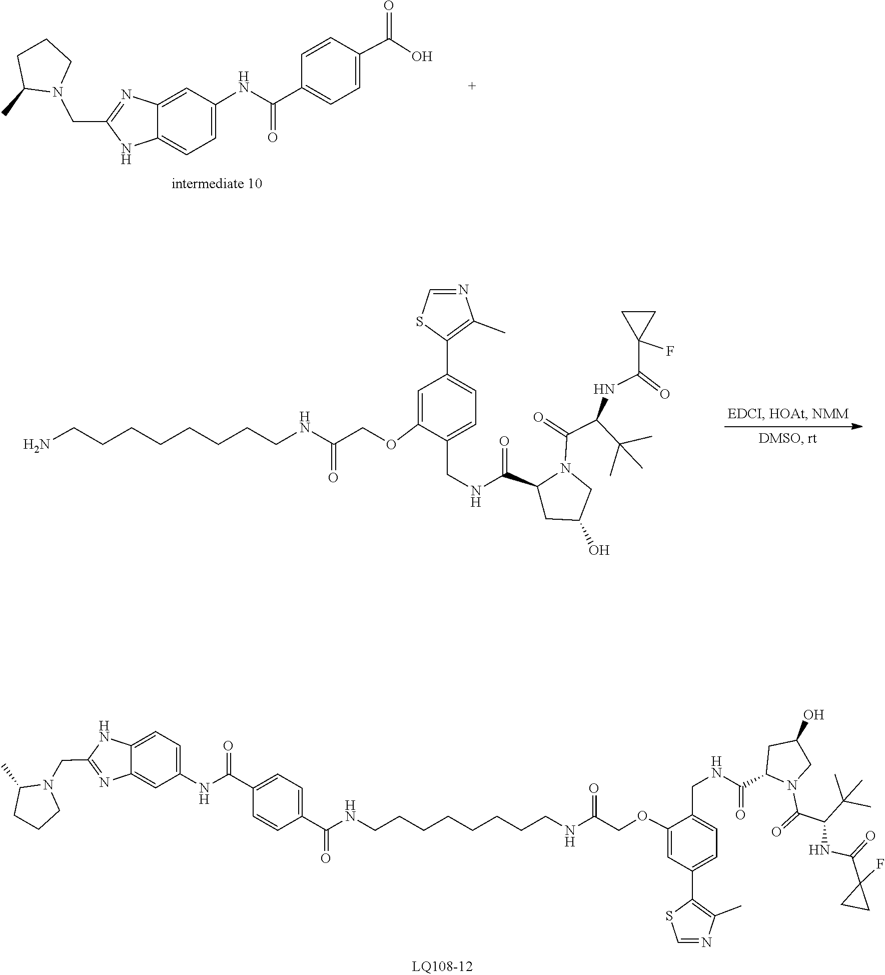 [SMILES: C[C@H]1CCCN1Cc1nc2cc(NC(=O)c3ccc(C(=O)O)cc3)ccc2[nH]1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCCCNC(=O)c2ccc(C(=O)Nc3ccc4[nH]c(CN5CCC[C@@H]5C)nc4c3)cc2)c1]

LQ108-12 was synthesized following the standard procedure for preparing LQ076-105 from intermediate 10 (10 mg, 0.02 mmol), (2S,4R)—N-(2-(2-((8-aminooctyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (15.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-12 was obtained as white solid in TFA salt form (17.9 mg, 68%). ¹H NMR (600 MHz, Methanol-d₄) δ 9.06 (s, 1H), 8.33 (d, J=2.0 Hz, 1H), 8.07-8.02 (m, 2H), 7.99-7.94 (m, 2H), 7.70 (d, J=8.8 Hz, 1H), 7.59 (dd, J=8.8, 2.0 Hz, 1H), 7.52 (d, J=7.7 Hz, 1H), 7.49 (dd, J=9.4, 3.4 Hz, 1H), 7.12 (dd, J=7.7, 1.6 Hz, 1H), 6.99 (d, J=1.6 Hz, 1H), 4.85 (d, J=14.7 Hz, 1H), 4.77-4.73 (m, 1H), 4.65-4.56 (m, 5H), 4.52-4.45 (m, 2H), 3.89-3.83 (m, 1H), 3.82-3.71 (m, 3H), 3.51-3.43 (m, 1H), 3.41 (t, J=7.2 Hz, 2H), 3.32-3.26 (m, 3H), 2.52 (s, 3H), 2.44-2.37 (m, 1H), 2.26-2.20 (m, 1H), 2.18-2.06 (m, 3H), 1.83 (s, 1H), 1.64 (p, J=7.2 Hz, 2H), 1.57 (p, J=7.1 Hz, 1H), 1.52 (d, J=6.5 Hz, 3H), 1.44-1.26 (m, 12H), 1.03 (s, 9H). HRMS m/z [M+H]⁺ calcd for C₅₇H₇₄FN₁₀O₈S⁺ 1077.5390, found 1077.5443.

### Synthesis of LQ108-141

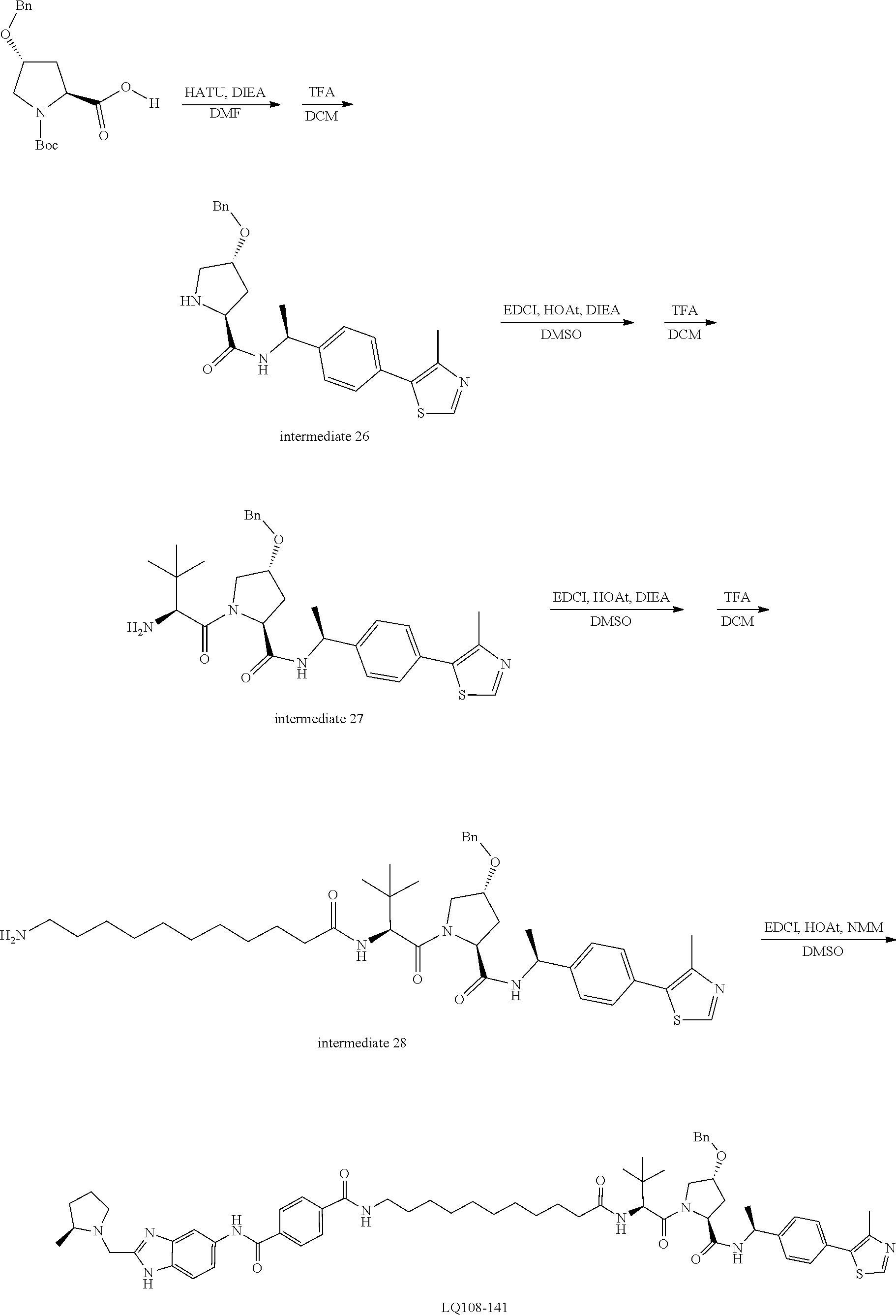

### Intermediate 26 (2S,4R)-4-(benzyloxy)-N—((S)-1-(4-(4-methylthiazol-5-yl)phenyl)ethyl)pyrrolidine-2-carboxamide

Intermediate 26 was synthesized according to the procedures for the preparation of intermediate 20 as a white solid in 63% yield. MS (ESI): m/z 422.6 [M+H]⁺.

### Intermediate 27 (2S,4R)-1-((S)-2-amino-3,3-dimethylbutanoyl)-4-(benzyloxy)-N—((S)-1-(4-(4-methylthiazol-5-yl)phenyl)ethyl)pyrrolidine-2-carboxamide

Intermediate 27 was synthesized according to the procedures for the preparation of intermediate 20 as a white solid in 74% yield. ¹H NMR (600 MHz, Methanol-d₄) δ 9.03 (s, 1H), 7.49-7.43 (m, 4H), 7.39-7.32 (m, 4H), 7.32-7.27 (m, 1H), 5.03 (q, J=7.1 Hz, 1H), 4.69 (dd, J=9.6, 7.6 Hz, 1H), 4.57 (s, 2H), 4.29 (t, J=4.0 Hz, 1H), 4.12 (s, 1H), 4.08-4.02 (m, 1H), 3.69 (dd, J=11.6, 3.7 Hz, 1H), 2.58-2.52 (m, 1H), 2.50 (s, 3H), 1.98-1.92 (m, 1H), 1.52 (d, J=7.1 Hz, 3H), 1.17 (s, 9H). MS (ESI): m/z 535.4 [M+H]⁺.

### Intermediate 28 (2S,4R)-1-((S)-2-(11-aminoundecanamido)-3,3-dimethylbutanoyl)-4-(benzyloxy)-N—((S)-1-(4-(4-methylthiazol-5-yl)phenyl)ethyl)pyrrolidine-2-carboxamide

Intermediate 28 was synthesized according to the procedures for the preparation of intermediate 20 as a white solid in 69% yield. ¹H NMR (600 MHz, Methanol-d₄) δ 8.93 (s, 1H), 7.48-7.42 (m, 4H), 7.36-7.30 (m, 4H), 7.29-7.25 (m, 1H), 5.02 (q, J=6.9 Hz, 1H), 4.72 (s, 1H), 4.62-4.47 (m, 3H), 4.29-4.24 (m, 2H), 3.71 (dd, J=11.6, 3.9 Hz, 1H), 2.92 (t, J=7.7 Hz, 2H), 2.50 (s, 3H), 2.44-2.36 (m, 1H), 2.35-2.20 (m, 2H), 2.02-1.96 (m, 1H), 1.70-1.56 (m, 5H), 1.52 (d, J=7.0 Hz, 3H), 1.44-1.29 (m, 12H), 1.07 (s, 9H). MS (ESI): m/z 718.3 [M+H]⁺.

LQ108-141 was synthesized following the standard procedure for preparing LQ076-105 from intermediate 10 (10 mg, 0.02 mmol), intermediate 28 (16.6 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-141 was obtained as white solid in TFA salt form (20.4 mg, 78%). ¹H NMR (600 MHz, Methanol-d₄) δ 9.09 (s, 1H), 8.34 (d, J=2.0 Hz, 1H), 8.08-8.02 (m, 2H), 7.98-7.94 (m, 2H), 7.70 (d, J=8.8 Hz, 1H), 7.61 (dd, J=8.8, 2.0 Hz, 1H), 7.48-7.42 (m, 4H), 7.34-7.29 (m, 4H), 7.29-7.24 (m, 1H), 5.01 (q, J=7.0 Hz, 1H), 4.86 (d, J=14.6 Hz, 1H), 4.72 (s, 1H), 4.65-4.55 (m, 3H), 4.51-4.46 (m, 1H), 4.29-4.22 (m, 2H), 3.79-3.66 (m, 3H), 3.49-3.38 (m, 4H), 2.50 (s, 3H), 2.43-2.36 (m, 2H), 2.33-2.20 (m, 2H), 2.19-2.05 (m, 2H), 2.01-1.95 (m, 1H), 1.87-1.79 (m, 1H), 1.70-1.54 (m, 5H), 1.53-1.49 (m, 6H), 1.45-1.26 (m, 11H), 1.06 (s, 9H). HRMS m/z [M+H]⁺ calcd for C₆₂H₈₀N₉O₆S⁺ 1078.5947, found 1078.5958.

### Synthesis of LQ108-142

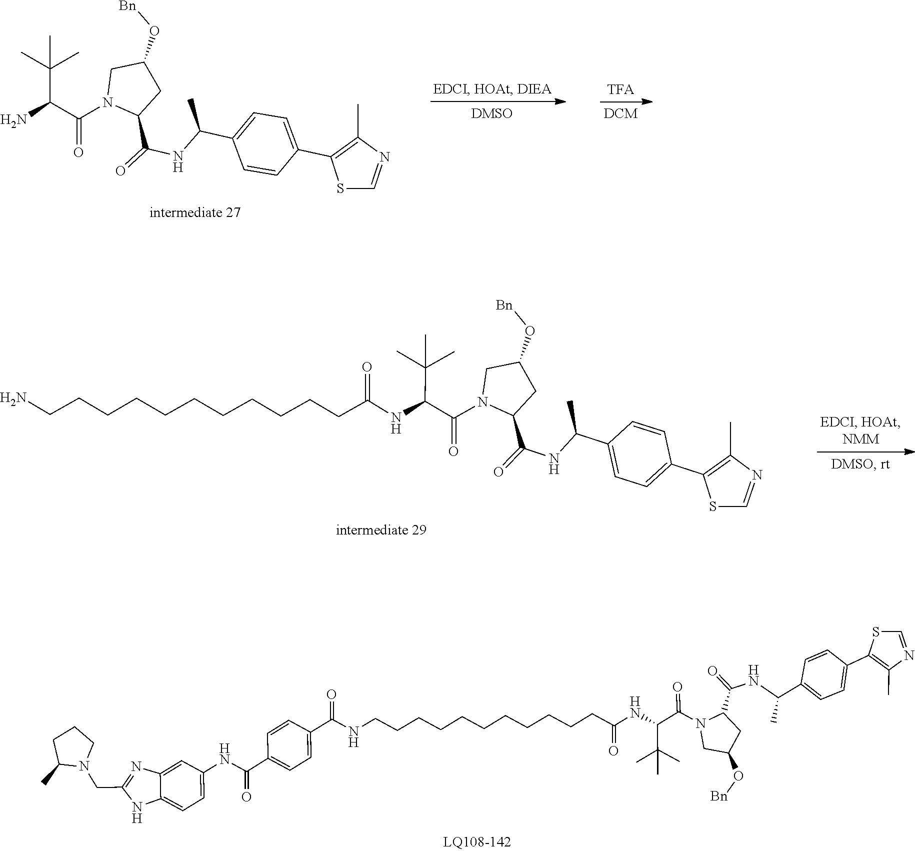

### Intermediate 29 (2S,4R)-1-((S)-2-(12-aminododecanamido)-3,3-dimethylbutanoyl)-4-(benzyloxy)-N—((S)-1-(4-(4-methylthiazol-5-yl)phenyl)ethyl)pyrrolidine-2-carboxamide

Intermediate 29 was synthesized according to the procedure for the preparation of intermediate 20 as a white solid in 77% yield. ¹H NMR (600 MHz, Methanol-d₄) δ 8.86 (s, 1H), 7.41-7.35 (m, 4H), 7.29-7.24 (m, 4H), 7.23-7.19 (m, 1H), 4.96 (q, J=6.8 Hz, 1H), 4.66 (s, 1H), 4.55-4.41 (m, 3H), 4.23-4.17 (m, 2H), 3.64 (dd, J=11.6, 3.9 Hz, 1H), 2.86 (t, J=7.7 Hz, 2H), 2.43 (s, 3H), 2.37-2.30 (m, 1H), 2.27-2.14 (m, 2H), 1.96-1.89 (m, 1H), 1.63-1.49 (m, 3H), 1.46 (d, J=7.0 Hz, 3H), 1.37-1.21 (m, 14H), 1.00 (s, 9H). MS (ESI): m/z 732.7 [M+H]⁺.

LQ108-142 was synthesized following the standard procedure for preparing LQ076-105 from intermediate 10 (10 mg, 0.02 mmol), intermediate 29 (17 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-142 was obtained as white solid in TFA salt form (20.8 mg, 79%). ¹H NMR (600 MHz, Methanol-d₄) δ 9.09 (s, 1H), 8.34 (d, J=2.0 Hz, 1H), 8.07-8.02 (m, 2H), 7.99-7.94 (m, 2H), 7.70 (d, J=8.8 Hz, 1H), 7.61 (dd, J=8.8, 2.0 Hz, 1H), 7.48-7.42 (m, 4H), 7.34-7.28 (m, 5H), 7.28-7.24 (m, 1H), 5.01 (q, J=7.0 Hz, 1H), 4.86 (d, J=14.6 Hz, 1H), 4.71 (s, 1H), 4.65-4.55 (m, 3H), 4.51-4.45 (m, 1H), 4.29-4.23 (m, 2H), 3.79-3.66 (m, 3H), 3.49-3.39 (m, 3H), 2.50 (s, 3H), 2.43-2.36 (m, 2H), 2.33-2.20 (m, 2H), 2.18-2.06 (m, 2H), 2.01-1.95 (m, 1H), 1.87-1.79 (m, 1H), 1.68-1.55 (m, 3H), 1.51 (d, J=7.5 Hz, 4H), 1.45-1.26 (m, 16H), 1.06 (s, 9H). HRMS m/z [M+H]⁺ calcd for C₆₃H₈₂N₉O₆S⁺ 1092.6103, found 1092.6113.

### Example 166

### Synthesis of LQ108-146

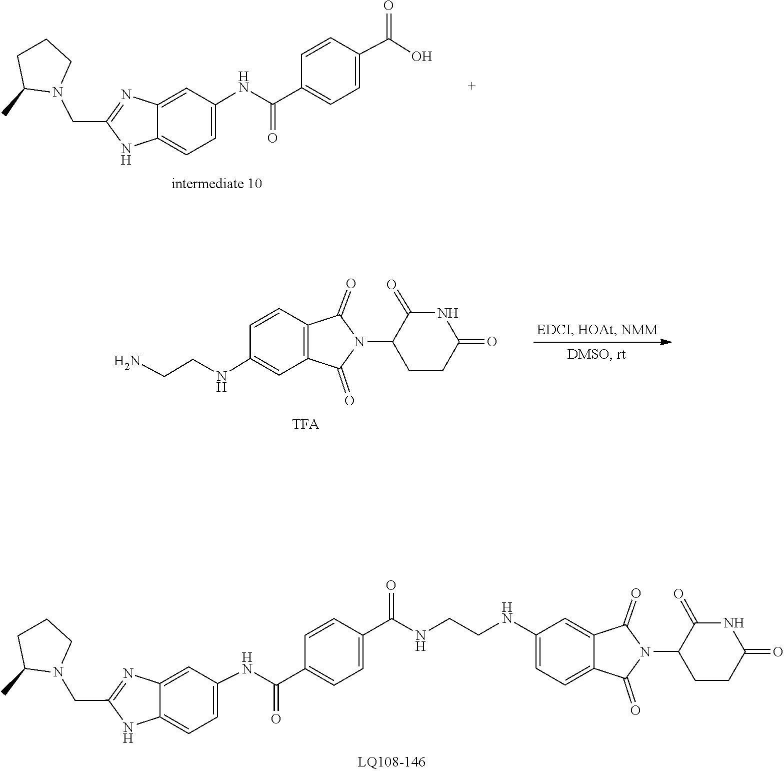 [SMILES: C[C@H]1CCCN1Cc1nc2cc(NC(=O)c3ccc(C(=O)O)cc3)ccc2[nH]1 ; NCCNc1ccc2c(c1)C(=O)N(C1CCC(=O)NC1=O)C2=O ; C[C@H]1CCCN1Cc1nc2cc(NC(=O)c3ccc(C(=O)NCCNc4ccc5c(c4)C(=O)N(C4CCC(=O)NC4=O)C5=O)cc3)ccc2[nH]1]

LQ108-146 was synthesized following the standard procedure for preparing LQ076-105 from intermediate 10 (10 mg, 0.02 mmol), 5-((2-aminoethyl)amino)-2-(2,6-dioxopiperidin-3-yl)isoindoline-1,3-dione (10.9 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-146 was obtained as yellow solid in TFA salt form (14.8 mg, 73%). ¹H NMR (600 MHz, Methanol-d₄) δ 8.16 (d, J=1.9 Hz, 1H), 7.93-7.89 (m, 2H), 7.84-7.78 (m, 2H), 7.55 (d, J=8.8 Hz, 1H), 7.46-7.40 (m, 2H), 6.97 (d, J=2.2 Hz, 1H), 6.82 (dd, J=8.4, 2.2 Hz, 1H), 4.92 (dd, J=12.6, 5.5 Hz, 1H), 4.68 (d, J=14.6 Hz, 1H), 4.44 (d, J=14.6 Hz, 1H), 3.67-3.59 (m, 2H), 3.55 (t, J=6.3 Hz, 2H), 3.43 (t, J=6.3 Hz, 2H), 3.37-3.31 (m, 1H), 2.77-2.69 (m, 1H), 2.64-2.53 (m, 2H), 2.32-2.23 (m, 1H), 2.07-1.93 (m, 3H), 1.75-1.66 (m, 1H), 1.39 (d, J=6.5 Hz, 3H). HRMS m/z [M+H]⁺ calcd for C₃₆H₃₇N₈O₆⁺ 677.2831, found 677.2797.

### Example 167

### Synthesis of LQ108-147

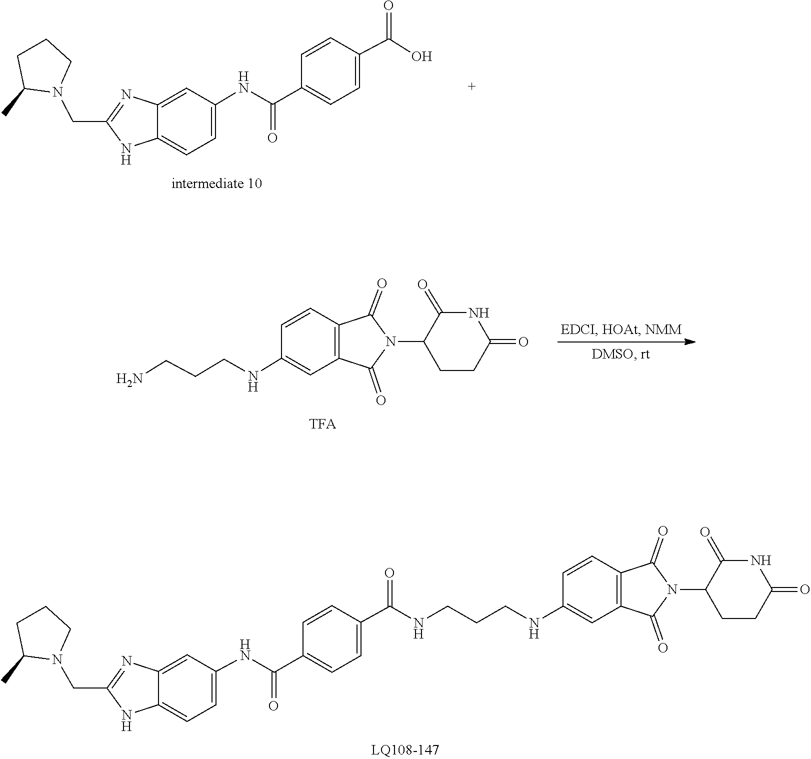 [SMILES: C[C@H]1CCCN1Cc1nc2cc(NC(=O)c3ccc(C(=O)O)cc3)ccc2[nH]1 ; NCCCNc1ccc2c(c1)C(=O)N(C1CCC(=O)NC1=O)C2=O ; C[C@H]1CCCN1Cc1nc2cc(NC(=O)c3ccc(C(=O)NCCCNc4ccc5c(c4)C(=O)N(C4CCC(=O)NC4=O)C5=O)cc3)ccc2[nH]1]

LQ108-147 was synthesized following the standard procedure for preparing LQ076-105 from intermediate 10 (10 mg, 0.02 mmol), 5-((3-aminopropyl)amino)-2-(2,6-dioxopiperidin-3-yl)isoindoline-1,3-dione (11.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-147 was obtained as yellow solid in TFA salt form (13.9 mg, 67%). ¹H NMR (600 MHz, Methanol-d₄) δ 8.17 (d, J=1.9 Hz, 1H), 7.96-7.91 (m, 2H), 7.87-7.83 (m, 2H), 7.57-7.53 (m, 1H), 7.48-7.42 (m, 2H), 6.90 (d, J=2.2 Hz, 1H), 6.76 (dd, J=8.4, 2.2 Hz, 1H), 4.93 (dd, J=12.5, 5.5 Hz, 1H), 4.67 (d, J=14.6 Hz, 1H), 4.44 (d, J=14.7 Hz, 1H), 3.66-3.59 (m, 2H), 3.45 (t, J=6.8 Hz, 2H), 3.38-3.31 (m, 1H), 3.24 (t, J=6.9 Hz, 2H), 2.78-2.69 (m, 1H), 2.66-2.55 (m, 2H), 2.32-2.24 (m, 1H), 2.09-1.95 (m, 3H), 1.89 (p, J=6.9 Hz, 2H), 1.75-1.66 (m, 1H), 1.39 (d, J=6.5 Hz, 3H). HRMS m/z [M+H]⁺ calcd for C₃₇H₃₉N₈O₆⁺ 691.2987, found 691.2999.

### Example 168

### Synthesis of LQ108-148

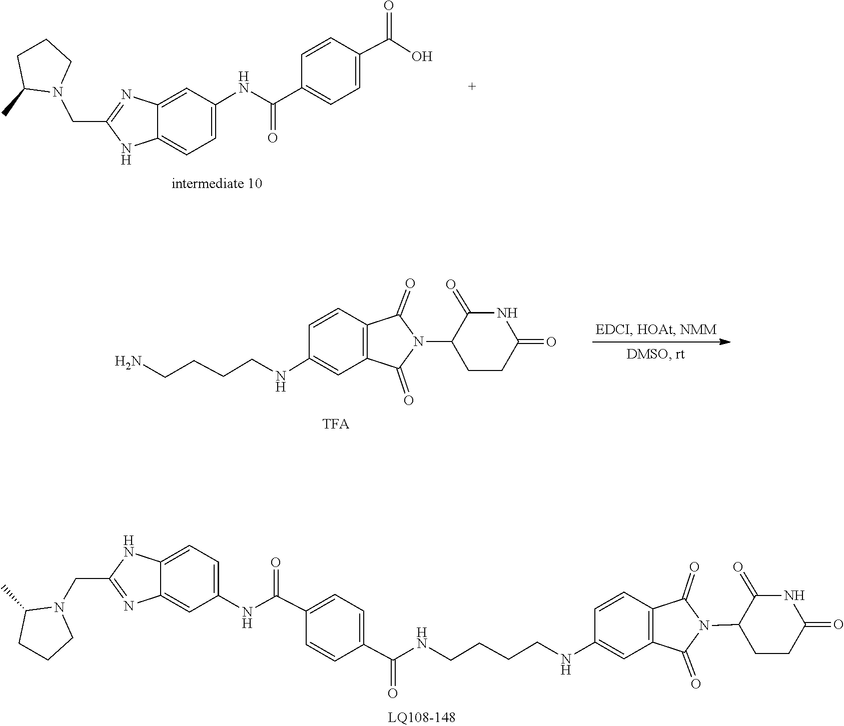 [SMILES: C[C@H]1CCCN1Cc1nc2cc(NC(=O)c3ccc(C(=O)O)cc3)ccc2[nH]1 ; NCCCCNc1ccc2c(c1)C(=O)N(C1CCC(=O)NC1=O)C2=O ; C[C@H]1CCCN1Cc1nc2cc(NC(=O)c3ccc(C(=O)NCCCCNc4ccc5c(c4)C(=O)N(C4CCC(=O)NC4=O)C5=O)cc3)ccc2[nH]1]

LQ108-148 was synthesized following the standard procedure for preparing LQ076-105 from intermediate 10 (10 mg, 0.02 mmol), 5-((4-aminobutyl)amino)-2-(2,6-dioxopiperidin-3-yl)isoindoline-1,3-dione (11.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-148 was obtained as yellow solid in TFA salt form (12.8 mg, 61%). ¹H NMR (600 MHz, Methanol-d₄) δ 8.29 (d, J=2.0 Hz, 1H), 8.06-8.01 (m, 2H), 7.95-7.91 (m, 2H), 7.70-7.66 (m, 1H), 7.58-7.52 (m, 2H), 6.99 (d, J=2.2 Hz, 1H), 6.85 (dd, J=8.4, 2.2 Hz, 1H), 5.04 (dd, J=12.6, 5.5 Hz, 1H), 4.79 (d, J=14.6 Hz, 1H), 4.55 (d, J=14.6 Hz, 1H), 3.78-3.71 (m, 2H), 3.51-3.43 (m, 3H), 3.31 (t, J=6.5 Hz, 2H), 2.87-2.79 (m, 1H), 2.75-2.66 (m, 2H), 2.44-2.37 (m, 1H), 2.19-2.05 (m, 2H), 1.87-1.75 (m, 6H), 1.51 (d, J=6.5 Hz, 3H). HRMS m/z [M+H]⁺ calcd for C₃₈H₄₁N₈O₆⁺ 705.3144, found 705.3141.

### Example 169

### Synthesis of LQ108-149

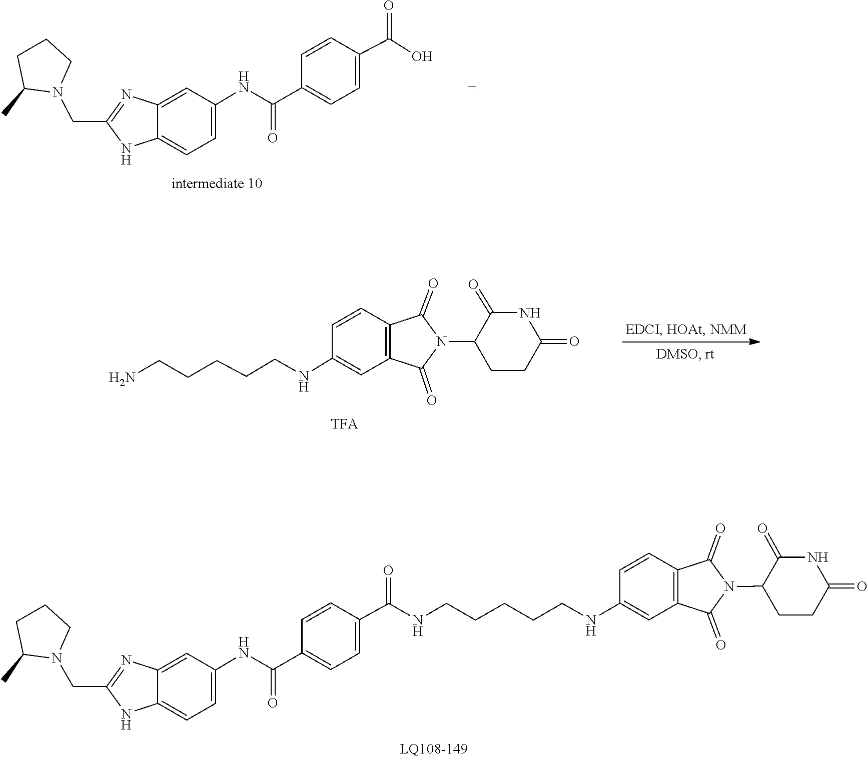 [SMILES: C[C@H]1CCCN1Cc1nc2cc(NC(=O)c3ccc(C(=O)O)cc3)ccc2[nH]1 ; NCCCCCNc1ccc2c(c1)C(=O)N(C1CCC(=O)NC1=O)C2=O ; C[C@H]1CCCN1Cc1nc2cc(NC(=O)c3ccc(C(=O)NCCCCCNc4ccc5c(c4)C(=O)N(C4CCC(=O)NC4=O)C5=O)cc3)ccc2[nH]1]

LQ108-149 was synthesized following the standard procedure for preparing LQ076-105 from intermediate 10 (10 mg, 0.02 mmol), 5-((5-aminopentyl)amino)-2-(2,6-dioxopiperidin-3-yl)isoindoline-1,3-dione (11.7 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-149 was obtained as yellow solid in TFA salt form (14.3 mg, 67%). ¹H NMR (600 MHz, Methanol-d₄) δ 8.29 (d, J=1.9 Hz, 1H), 8.06-8.01 (m, 2H), 7.96-7.91 (m, 2H), 7.68 (d, J=8.8 Hz, 1H), 7.57-7.52 (m, 2H), 6.98 (d, J=2.2 Hz, 1H), 6.84 (dd, J=8.4, 2.2 Hz, 1H), 5.03 (dd, J=12.4, 5.5 Hz, 1H), 4.79 (d, J=14.7 Hz, 1H), 4.55 (d, J=14.6 Hz, 1H), 3.78-3.71 (m, 2H), 3.50-3.43 (m, 3H), 3.27 (t, J=6.9 Hz, 2H), 2.86-2.77 (m, 1H), 2.74-2.64 (m, 2H), 2.44-2.36 (m, 1H), 2.19-2.05 (m, 2H), 1.86-1.70 (m, 6H), 1.60-1.53 (m, 2H), 1.51 (d, J=6.5 Hz, 3H). HRMS m/z [M+H]⁺ calcd for C₃₉H₄₃N₈O₆⁺ 719.3300, found 719.3295.

### Example 170

### Synthesis of LQ108-150

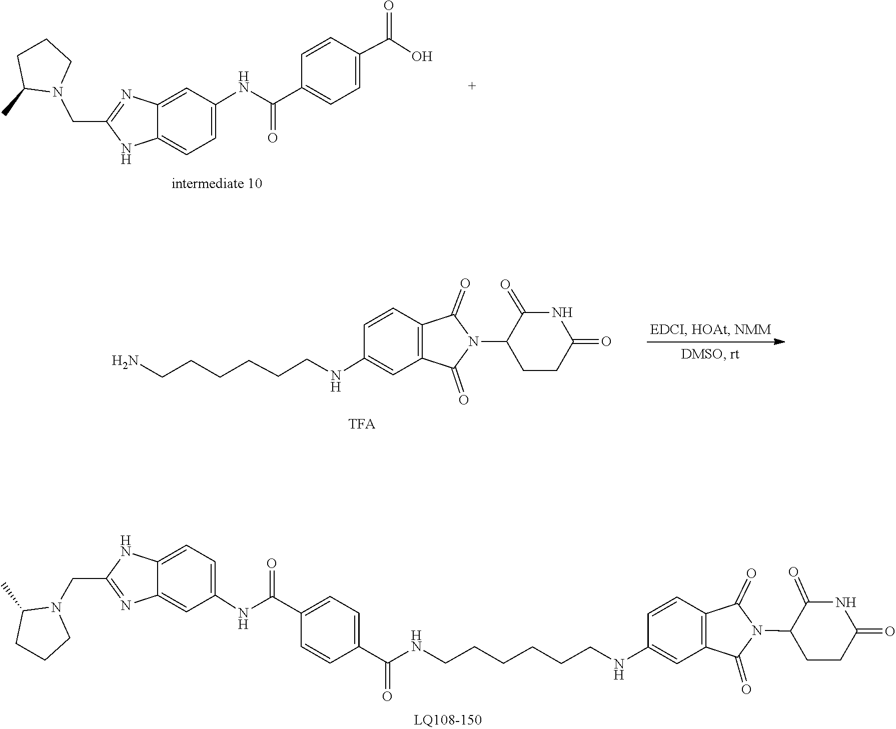 [SMILES: C[C@H]1CCCN1Cc1nc2cc(NC(=O)c3ccc(C(=O)O)cc3)ccc2[nH]1 ; NCCCCCCNc1ccc2c(c1)C(=O)N(C1CCC(=O)NC1=O)C2=O ; C[C@H]1CCCN1Cc1nc2cc(NC(=O)c3ccc(C(=O)NCCCCCCNc4ccc5c(c4)C(=O)N(C4CCC(=O)NC4=O)C5=O)cc3)ccc2[nH]1]

LQ108-150 was synthesized following the standard procedure for preparing LQ076-105 from intermediate 10 (10 mg, 0.02 mmol), 5-((6-aminohexyl)amino)-2-(2,6-dioxopiperidin-3-yl)isoindoline-1,3-dione (12 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-150 was obtained as yellow solid in TFA salt form (15.6 mg, 73%). ¹H NMR (600 MHz, Methanol-d₄) δ 8.30 (d, J=2.0 Hz, 1H), 8.07-8.01 (m, 2H), 7.97-7.92 (m, 2H), 7.69 (d, J=8.8 Hz, 1H), 7.59-7.52 (m, 2H), 6.97 (d, J=2.2 Hz, 1H), 6.83 (dd, J=8.4, 2.2 Hz, 1H), 5.03 (dd, J=12.6, 5.5 Hz, 1H), 4.81 (d, J=14.6 Hz, 1H), 4.57 (d, J=14.6 Hz, 1H), 3.78-3.71 (m, 2H), 3.51-3.42 (m, 3H), 3.23 (t, J=7.0 Hz, 2H), 2.87-2.78 (m, 1H), 2.75-2.65 (m, 2H), 2.45-2.36 (m, 1H), 2.19-2.05 (m, 2H), 1.88-1.78 (m, 1H), 1.75-1.65 (m, 4H), 1.57-1.46 (m, 8H). HRMS m/z [M+H]⁺ calcd for C₄₀H₄₅N₈O₆⁺ 733.3457, found 733.3485.

### Example 171

### Synthesis of LQ108-151

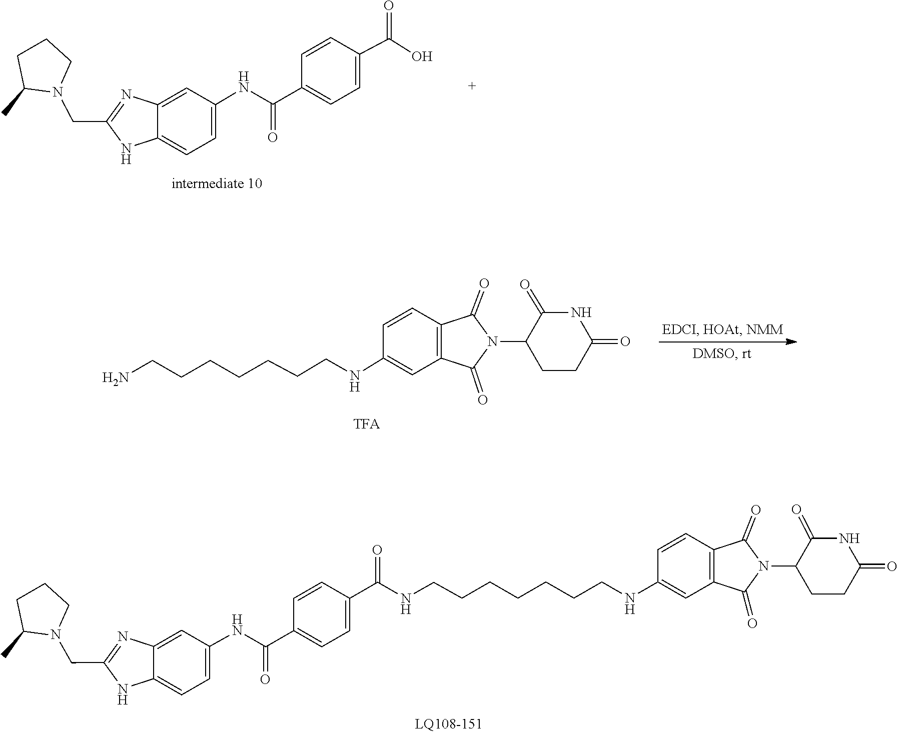 [SMILES: C[C@H]1CCCN1Cc1nc2cc(NC(=O)c3ccc(C(=O)O)cc3)ccc2[nH]1 ; NCCCCCCCNc1ccc2c(c1)C(=O)N(C1CCC(=O)NC1=O)C2=O ; C[C@H]1CCCN1Cc1nc2cc(NC(=O)c3ccc(C(=O)NCCCCCCCNc4ccc5c(c4)C(=O)N(C4CCC(=O)NC4=O)C5=O)cc3)ccc2[nH]1]

LQ108-151 was synthesized following the standard procedure for preparing LQ076-105 from intermediate 10 (10 mg, 0.02 mmol), 5-((7-aminoheptyl)amino)-2-(2,6-dioxopiperidin-3-yl)isoindoline-1,3-dione (12.2 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-151 was obtained as yellow solid in TFA salt form (17.3 mg, 80%). ¹H NMR (600 MHz, Methanol-d₄) δ 8.29 (d, J=1.9 Hz, 1H), 8.07-8.02 (m, 2H), 7.98-7.93 (m, 2H), 7.68 (d, J=8.8 Hz, 1H), 7.57-7.53 (m, 2H), 6.97 (d, J=2.2 Hz, 1H), 6.83 (dd, J=8.4, 2.2 Hz, 1H), 5.04 (dd, J=12.4, 5.5 Hz, 1H), 4.80 (d, J=14.7 Hz, 1H), 4.56 (d, J=14.6 Hz, 1H), 3.78-3.70 (m, 2H), 3.50-3.41 (m, 3H), 3.22 (t, J=7.1 Hz, 2H), 2.88-2.79 (m, 1H), 2.75-2.65 (m, 2H), 2.44-2.36 (m, 1H), 2.19-2.06 (m, 2H), 1.87-1.78 (m, 1H), 1.72-1.64 (m, 4H), 1.51 (d, J=6.5 Hz, 3H), 1.50-1.43 (m, 7H). HRMS m/z [M+H]⁺ calcd for C₄₁H₄₇N₈O₆⁺ 747.3613, found 747.3638.

### Example 172

### Synthesis of LQ108-152

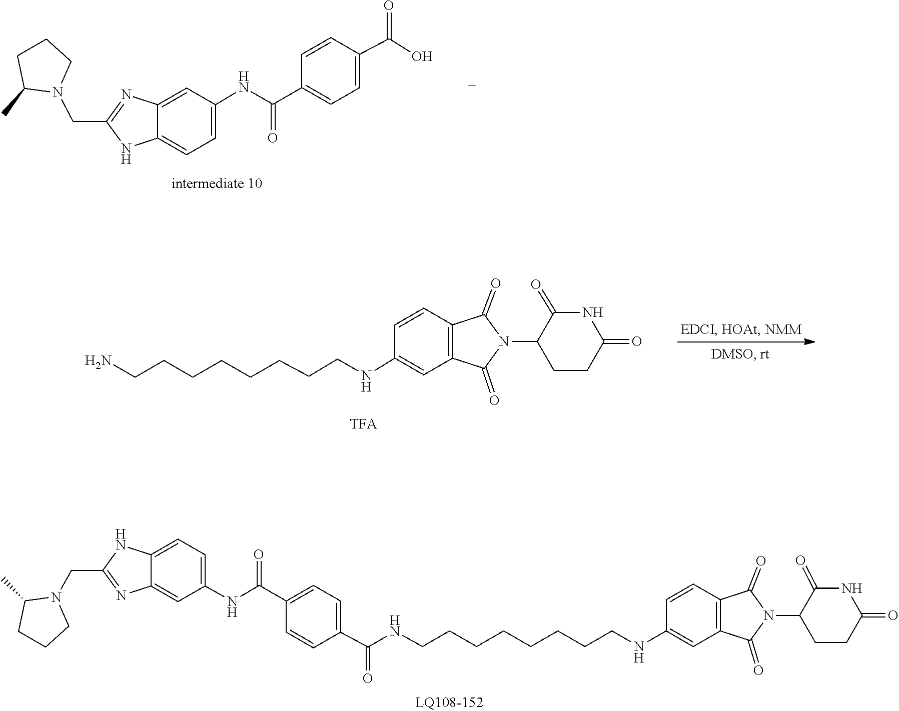 [SMILES: C[C@H]1CCCN1Cc1nc2cc(NC(=O)c3ccc(C(=O)O)cc3)ccc2[nH]1 ; NCCCCCCCCNc1ccc2c(c1)C(=O)N(C1CCC(=O)NC1=O)C2=O ; C[C@H]1CCCN1Cc1nc2cc(NC(=O)c3ccc(C(=O)NCCCCCCCCNc4ccc5c(c4)C(=O)N(C4CCC(=O)NC4=O)C5=O)cc3)ccc2[nH]1]

LQ108-152 was synthesized following the standard procedure for preparing LQ076-105 from intermediate 10 (10 mg, 0.02 mmol), 5-((8-aminooctyl)amino)-2-(2,6-dioxopiperidin-3-yl)isoindoline-1,3-dione (12.6 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-152 was obtained as yellow solid in TFA salt form (16.4 mg, 74%). ¹H NMR (600 MHz, Methanol-d₄) δ 8.29 (d, J=2.0 Hz, 1H), 8.07-8.02 (m, 2H), 7.98-7.94 (m, 2H), 7.68 (dd, J=8.7, 0.6 Hz, 1H), 7.57-7.53 (m, 2H), 6.97 (d, J=2.2 Hz, 1H), 6.83 (dd, J=8.4, 2.2 Hz, 1H), 5.04 (dd, J=12.7, 5.5 Hz, 1H), 4.80 (d, J=14.6 Hz, 1H), 4.56 (d, J=14.6 Hz, 1H), 3.78-3.71 (m, 2H), 3.50-3.40 (m, 3H), 3.21 (t, J=7.1 Hz, 2H), 2.88-2.80 (m, 1H), 2.76-2.65 (m, 2H), 2.45-2.36 (m, 1H), 2.20-2.06 (m, 2H), 1.86-1.79 (m, 1H), 1.71-1.64 (m, 4H), 1.51 (d, J=6.5 Hz, 3H), 1.49-1.40 (m, 9H). HRMS m/z [M+H]⁺ calcd for C₄₂H₄₉N₈O₆⁺ 761.3770, found 761.3764.

### Example 173

### Synthesis of LQ108-153

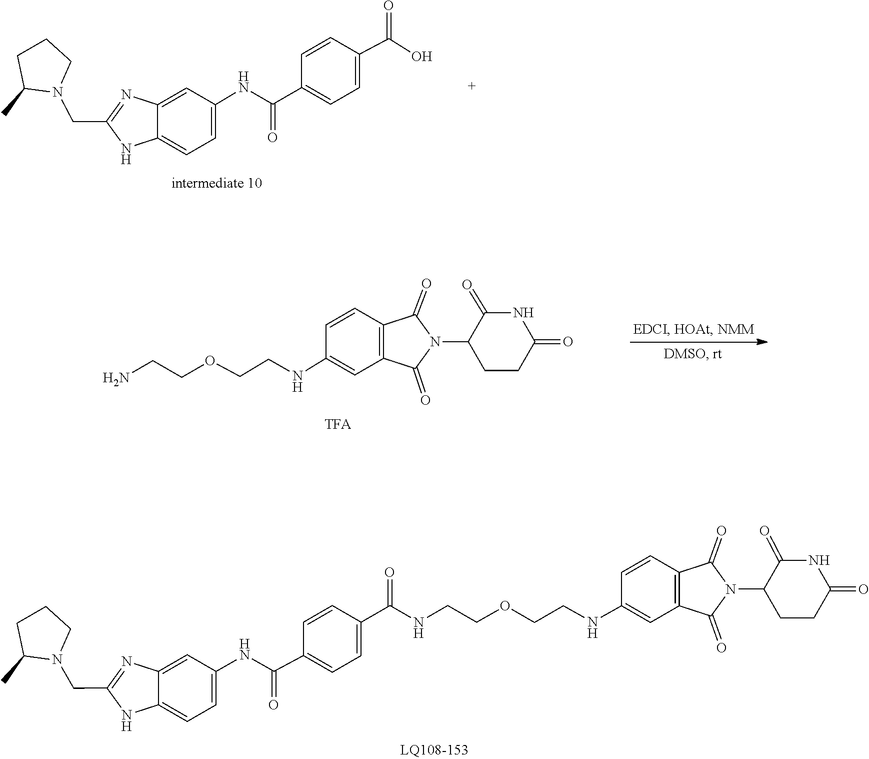 [SMILES: C[C@H]1CCCN1Cc1nc2cc(NC(=O)c3ccc(C(=O)O)cc3)ccc2[nH]1 ; NCCOCCNc1ccc2c(c1)C(=O)N(C1CCC(=O)NC1=O)C2=O ; C[C@H]1CCCN1Cc1nc2cc(NC(=O)c3ccc(C(=O)NCCOCCNc4ccc5c(c4)C(=O)N(C4CCC(=O)NC4=O)C5=O)cc3)ccc2[nH]1]

LQ108-153 was synthesized following the standard procedure for preparing LQ076-105 from intermediate 10 (10 mg, 0.02 mmol), 5-((2-(2-aminoethoxy)ethyl)amino)-2-(2,6-dioxopiperidin-3-yl)isoindoline-1,3-dione (11.8 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-153 was obtained as yellow solid in TFA salt form (15.8 mg, 74%). ¹H NMR (600 MHz, Methanol-d₄) δ 8.18 (d, J=1.9 Hz, 1H), 7.90-7.86 (m, 2H), 7.79-7.75 (m, 2H), 7.58-7.54 (m, 1H), 7.46 (dd, J=8.7, 2.0 Hz, 1H), 7.38 (d, J=8.4 Hz, 1H), 6.87 (d, J=2.2 Hz, 1H), 6.74 (dd, J=8.4, 2.2 Hz, 1H), 4.85 (dd, J=12.4, 5.4 Hz, 1H), 4.68 (d, J=14.6, 1H), 4.44 (d, J=14.6, 1H), 3.68-3.58 (m, 6H), 3.54-3.48 (m, 2H), 3.39-3.30 (m, 3H), 2.63-2.43 (m, 3H), 2.33-2.24 (m, 1H), 2.09-1.89 (m, 3H), 1.75-1.67 (m, 1H), 1.40 (d, J=6.5 Hz, 3H). HRMS m/z [M+H]⁺ calcd for C₃₈H₄₁N₈O₇⁺ 721.3093, found 721.3121.

### Example 174

### Synthesis of LQ108-154

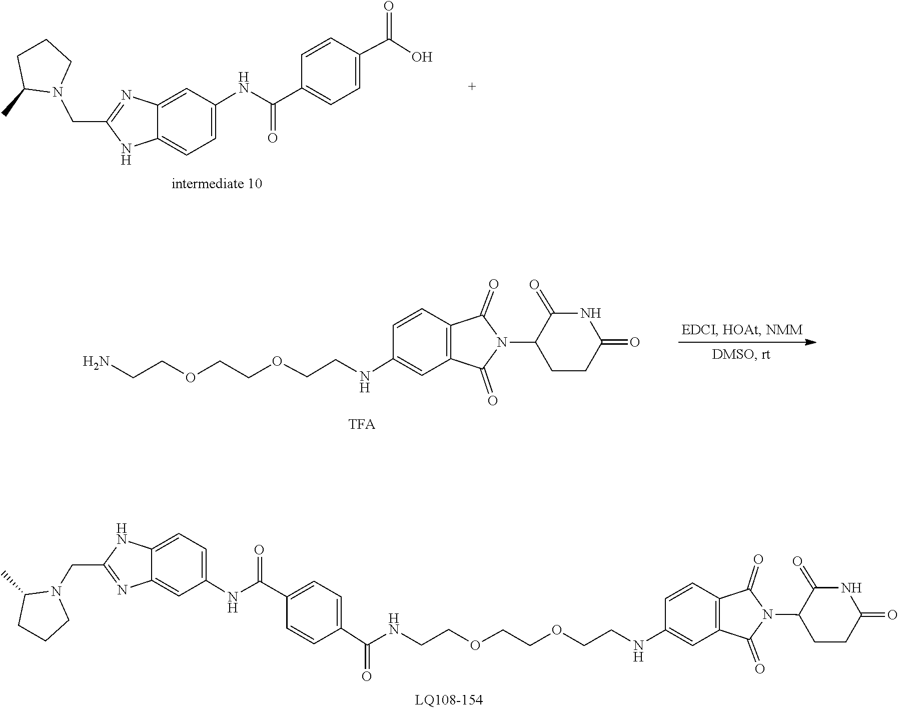 [SMILES: C[C@H]1CCCN1Cc1nc2cc(NC(=O)c3ccc(C(=O)O)cc3)ccc2[nH]1 ; NCCOCCOCCNc1ccc2c(c1)C(=O)N(C1CCC(=O)NC1=O)C2=O ; C[C@H]1CCCN1Cc1nc2cc(NC(=O)c3ccc(C(=O)NCCOCCOCCNc4ccc5c(c4)C(=O)N(C4CCC(=O)NC4=O)C5=O)cc3)ccc2[nH]1]

LQ108-154 was synthesized following the standard procedure for preparing LQ076-105 from intermediate 10 (10 mg, 0.02 mmol), 5-((2-(2-(2-aminoethoxy)ethoxy)ethyl)amino)-2-(2,6-dioxopiperidin-3-yl)isoindoline-1,3-dione (12.7 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-154 was obtained as yellow solid in TFA salt form (16.6 mg, 75%). ¹H NMR (600 MHz, Methanol-d₄) δ 8.28 (d, J=2.0 Hz, 1H), 8.03-7.97 (m, 2H), 7.97-7.92 (m, 2H), 7.67 (d, J=8.7 Hz, 1H), 7.55 (dd, J=8.8, 2.0 Hz, 1H), 7.51 (d, J=8.3 Hz, 1H), 6.97 (d, J=2.2 Hz, 1H), 6.83 (dd, J=8.4, 2.2 Hz, 1H), 4.97 (dd, J=12.6, 5.5 Hz, 1H), 4.81 (d, J=14.7 Hz, 1H), 4.57 (d, J=14.7, 1H), 3.79-3.68 (m, 11H), 3.63 (t, J=5.4 Hz, 2H), 3.51-3.43 (m, 1H), 3.37 (t, J=5.3 Hz, 2H), 2.78-2.70 (m, 1H), 2.66-2.58 (m, 2H), 2.44-2.37 (m, 1H), 2.20-2.07 (m, 2H), 2.05-1.99 (m, 1H), 1.87-1.79 (m, 1H), 1.52 (d, J=6.5 Hz, 3H). HRMS m/z [M+H]+ calcd for C₄₀H₄₅N₈O₈⁺ 765.3355, found 765.3390.

### Example 175

### Synthesis of LQ108-155

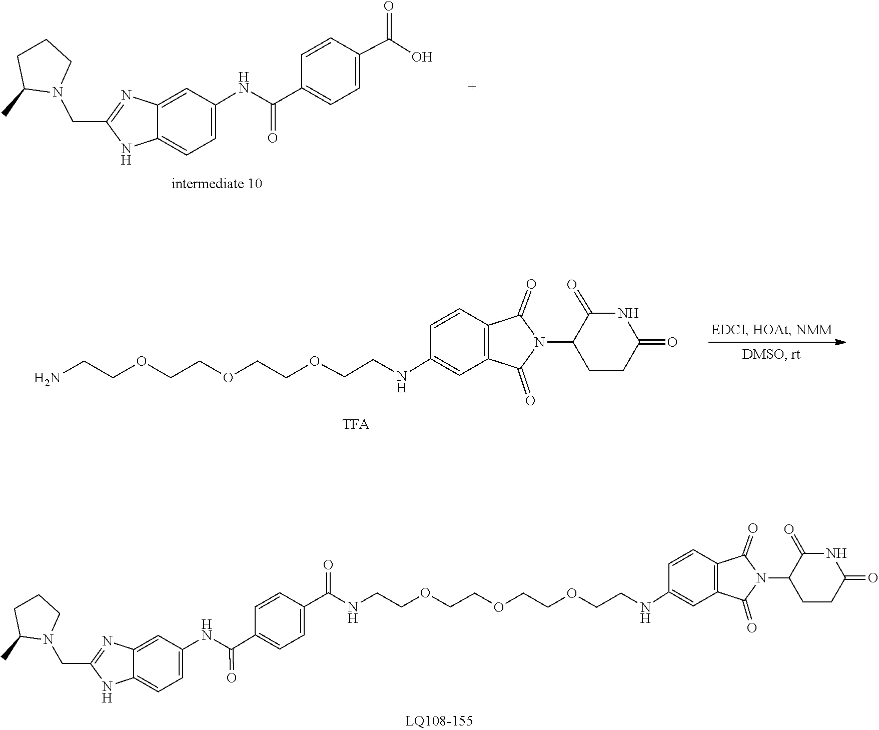 [SMILES: C[C@H]1CCCN1Cc1nc2cc(NC(=O)c3ccc(C(=O)O)cc3)ccc2[nH]1 ; NCCOCCOCCOCCNc1ccc2c(c1)C(=O)N(C1CCC(=O)NC1=O)C2=O ; C[C@H]1CCCN1Cc1nc2cc(NC(=O)c3ccc(C(=O)NCCOCCOCCOCCNc4ccc5c(c4)C(=O)N(C4CCC(=O)NC4=O)C5=O)cc3)ccc2[nH]1]

LQ108-155 was synthesized following the standard procedure for preparing LQ076-105 from intermediate 10 (10 mg, 0.02 mmol), 5-((2-(2-(2-(2-aminoethoxy)ethoxy)ethoxy)ethyl)amino)-2-(2,6-dioxopiperidin-3-yl)isoindoline-1,3-dione (13.5 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-155 was obtained as yellow solid in TFA salt form (17.8 mg, 77%). ¹H NMR (600 MHz, Methanol-d₄) δ 8.28 (d, J=1.9 Hz, 1H), 8.04-8.01 (m, 2H), 7.98-7.93 (m, 2H), 7.68-7.65 (m, 1H), 7.54 (dd, J=8.7, 2.0 Hz, 1H), 7.49 (d, J=8.3 Hz, 1H), 6.97 (d, J=2.2 Hz, 1H), 6.83 (dd, J=8.4, 2.2 Hz, 1H), 5.01 (dd, J=12.7, 5.5 Hz, 1H), 4.80 (d, J=14.6 Hz, 1H), 4.56 (d, J=14.7 Hz, 1H), 3.78-3.60 (m, 17H), 3.51-3.43 (m, 1H), 3.37 (t, J=5.4 Hz, 2H), 2.86-2.77 (m, 1H), 2.73-2.62 (m, 2H), 2.45-2.36 (m, 1H), 2.20-2.03 (m, 2H), 1.87-1.78 (m, 1H), 1.52 (d, J=6.5 Hz, 3H). HRMS m/z [M+H]⁺ calcd for C₄₂H₄₉N₈O₈⁺ 809.3617, found 809.3643.

### Example 176

### Synthesis of LQ108-156

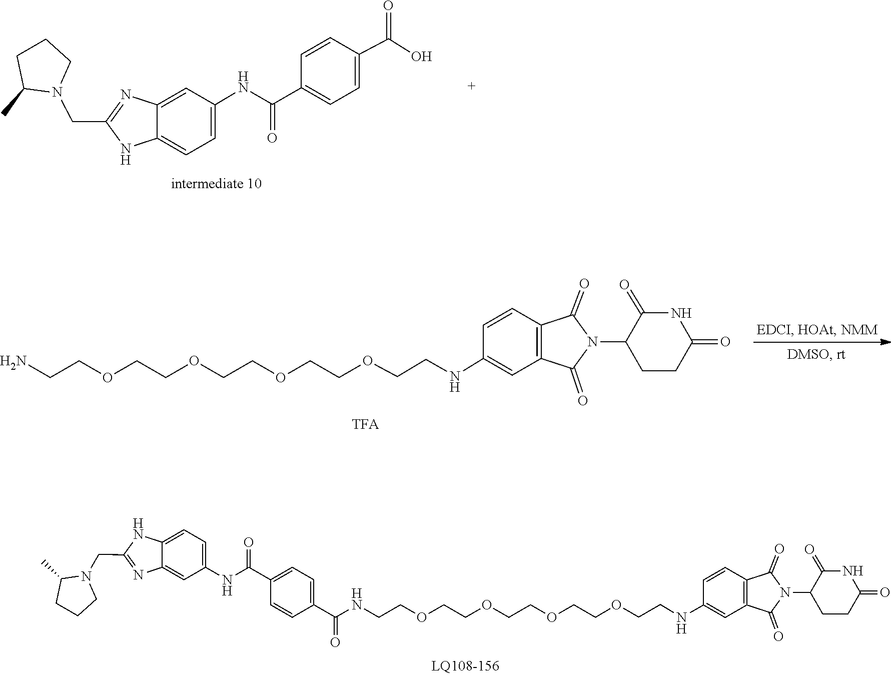 [SMILES: C[C@H]1CCCN1Cc1nc2cc(NC(=O)c3ccc(C(=O)O)cc3)ccc2[nH]1 ; NCCOCCOCCOCCOCCNc1ccc2c(c1)C(=O)N(C1CCC(=O)NC1=O)C2=O ; C[C@H]1CCCN1Cc1nc2cc(NC(=O)c3ccc(C(=O)NCCOCCOCCOCCOCCNc4ccc5c(c4)C(=O)N(C4CCC(=O)NC4=O)C5=O)cc3)ccc2[nH]1]

LQ108-156 was synthesized following the standard procedure for preparing LQ076-105 from intermediate 10 (10 mg, 0.02 mmol), 5-((14-amino-3,6,9,12-tetraoxatetradecyl)amino)-2-(2,6-dioxopiperidin-3-yl)isoindoline-1,3-dione (14.4 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-156 was obtained as yellow solid in TFA salt form (16.9 mg, 71%). ¹H NMR (600 MHz, Methanol-d₄) δ 8.29 (d, J=2.0 Hz, 1H), 8.06-8.03 (m, 2H), 7.99-7.95 (m, 2H), 7.67 (d, J=8.8 Hz, 1H), 7.56 (dd, J=8.8, 2.0 Hz, 1H), 7.49 (dd, J=8.3, 1.1 Hz, 1H), 6.98 (dd, J=2.2, 1.0 Hz, 1H), 6.84-6.80 (m, 1H), 5.03 (dd, J=12.7, 5.5 Hz, 1H), 4.81 (d, J=14.7, 1H), 4.57 (d, J=14.6, 1H), 3.79-3.71 (m, 2H), 3.71-3.59 (m, 19H), 3.50-3.43 (m, 1H), 3.39-3.34 (m, 2H), 2.88-2.80 (m, 1H), 2.75-2.64 (m, 2H), 2.44-2.37 (m, 1H), 2.19-2.05 (m, 2H), 1.87-1.78 (m, 1H), 1.51 (d, J=6.5 Hz, 3H). HRMS m/z [M+H]⁺ calcd for C₄₄H₅₃N₈O₁₀⁺ 853.3879, found 853.3871.

### Example 177

### Synthesis of LQ108-157

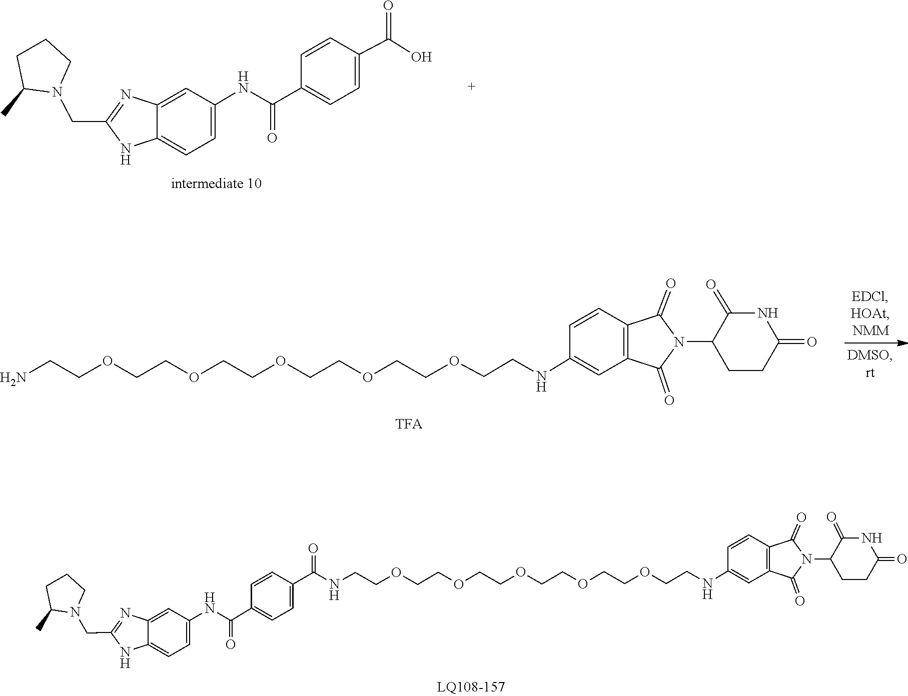 [SMILES: C[C@H]1CCCN1Cc1nc2cc(NC(=O)c3ccc(C(=O)O)cc3)ccc2[nH]1 ; NCCOCCOCCOCCOCCOCCNc1ccc2c(c1)C(=O)N(C1CCC(=O)NC1=O)C2=O ; C[C@H]1CCCN1Cc1nc2cc(NC(=O)c3ccc(C(=O)NCCOCCOCCOCCOCCOCCNc4ccc5c(c4)C(=O)N(C4CCC(=O)NC4=O)C5=O)cc3)ccc2[nH]1]

LQ108-157 was synthesized following the standard procedure for preparing LQ076-105 from intermediate 10 (10 mg, 0.02 mmol), 5-((17-amino-3,6,9,12,15-pentaoxaheptadecyl)amino)-2-(2,6-dioxopiperidin-3-yl)isoindoline-1,3-dione (15.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ108-157 was obtained as yellow solid in TFA salt form (16.3 mg, 65%). ¹H NMR (600 MHz, Methanol-d₄) δ 8.29 (d, J=1.9 Hz, 1H), 8.07-8.02 (m, 2H), 8.01-7.96 (m, 2H), 7.66 (d, J=8.8 Hz, 1H), 7.55 (dd, J=8.7, 2.0 Hz, 1H), 7.51 (dd, J=8.6, 7.1 Hz, 1H), 7.04 (d, J=8.5 Hz, 1H), 7.01 (d, J=7.1 Hz, 1H), 5.04 (dd, J=12.8, 5.5 Hz, 1H), 4.81 (d, J=14.6 Hz, 1H), 4.57 (d, J=14.6 Hz, 1H), 3.78-3.57 (m, 25H), 3.49-3.43 (m, 3H), 2.89-2.81 (m, 1H), 2.76-2.66 (m, 2H), 2.43-2.36 (m, 1H), 2.20-2.07 (m, 2H), 1.87-1.78 (m, 1H), 1.51 (d, J=6.5 Hz, 3H). HRMS m/z [M+H]⁺ calcd for C₄₆H₅₇N₈O₁₁⁺ 897.4141, found 8974174.

### Example 178

### Synthesis of LQ118-23

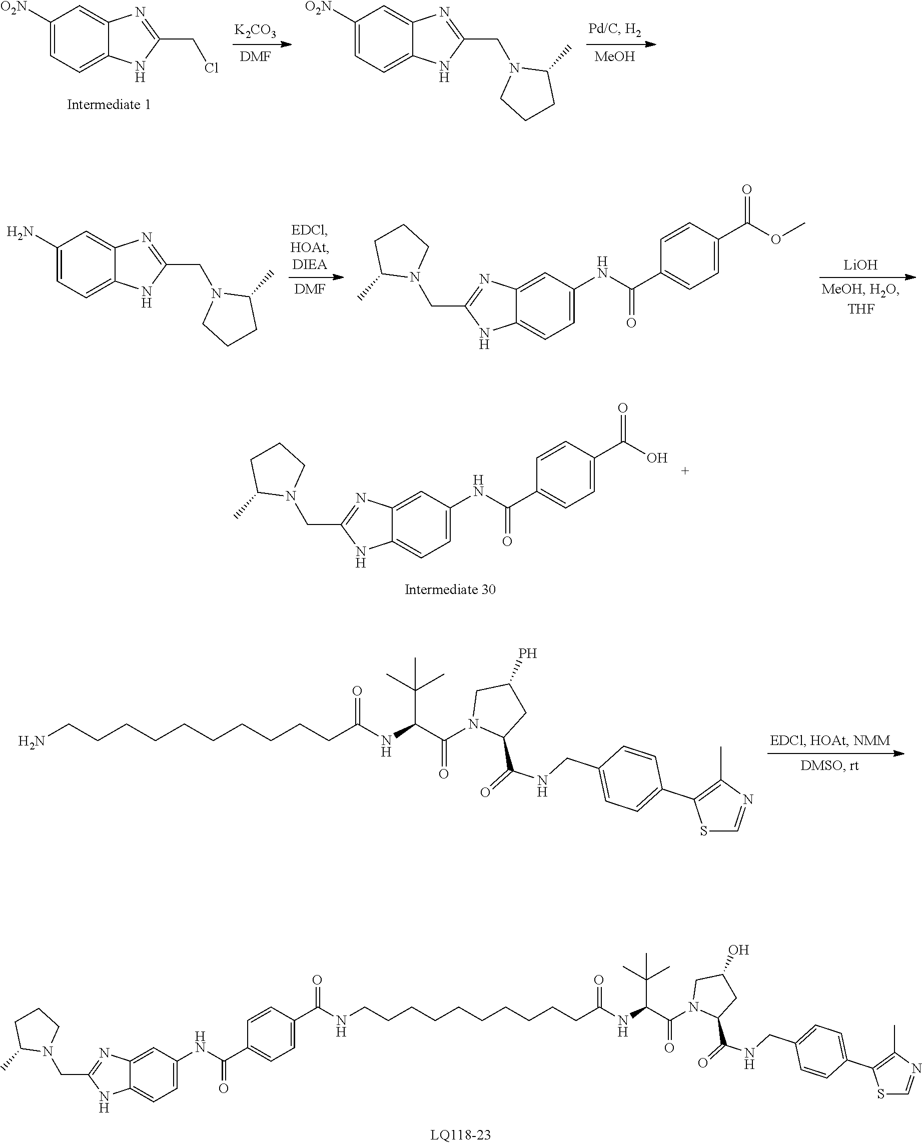

### Intermediate 30 (R)-4-((2-((2-methylpyrrolidin-1-yl)methyl)-1H-benzo[d]imidazol-5-yl)carbamoyl)benzoic acid

Intermediate 30 was synthesized according to the procedures for the preparation of intermediate 10 as a white solid in yield. ¹H NMR (600 MHz, Methanol-d₄) δ 8.32 (d, J=2.0 Hz, 1H), 8.20-8.14 (m, 2H), 8.08-8.02 (m, 2H), 7.70 (d, J=8.8 Hz, 1H), 7.60 (dd, J=8.8, 2.0 Hz, 1H), 4.85 (d, J=14.6 Hz, 1H), 4.62 (d, J=14.6 Hz, 1H), 3.80-3.69 (m, 2H), 3.50-3.43 (m, 1H), 2.44-2.35 (m, 1H), 2.20-2.05 (m, 2H), 1.88-1.78 (m, 1H), 1.51 (d, J=6.5 Hz, 3H). MS (ESI): m/z 379.7 [M+H]⁺.

LQ118-23 was synthesized following the standard procedure for preparing LQ076-105 from intermediate 30 (10 mg, 0.02 mmol), (2S,4R)-1-((S)-2-(11-aminoundecanamido)-3,3-dimethylbutanoyl)-4-hydroxy-N-(4-(4-methylthiazol-5-yl)benzyl)pyrrolidine-2-carboxamide (15.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ118-23 was obtained as white solid in TFA salt form (16.3 mg, 65%). ¹H NMR (600 MHz, Methanol-d₄) δ 9.03 (s, 1H), 8.31 (d, J=2.0 Hz, 1H), 8.08-8.03 (m, 2H), 7.98-7.95 (m, 2H), 7.69 (d, J=8.8 Hz, 1H), 7.58 (dd, J=8.7, 2.0 Hz, 1H), 7.51-7.46 (m, 2H), 7.45-7.42 (m, 2H), 4.82 (d, J=14.6 Hz, 1H), 4.65 (s, 1H), 4.62-4.50 (m, 4H), 4.38 (d, J=15.5 Hz, 1H), 3.95-3.90 (m, 1H), 3.82 (dd, J=11.0, 3.9 Hz, 1H), 3.77-3.71 (m, 2H), 3.50-3.39 (m, 3H), 2.50 (s, 3H), 2.43-2.36 (m, 1H), 2.34-2.21 (m, 3H), 2.18-2.07 (m, 2H), 1.87-1.79 (m, 1H), 1.69-1.58 (m, 5H), 1.51 (d, J=6.5 Hz, 3H), 1.46-1.31 (m, 12H), 1.05 (s, 9H). HRMS m/z [M+H]⁺ calcd for C₅₄H₇₂N₉O₆S⁺ 974.5321, found 974.5323.

### Example 179

### Synthesis of LQ118-24

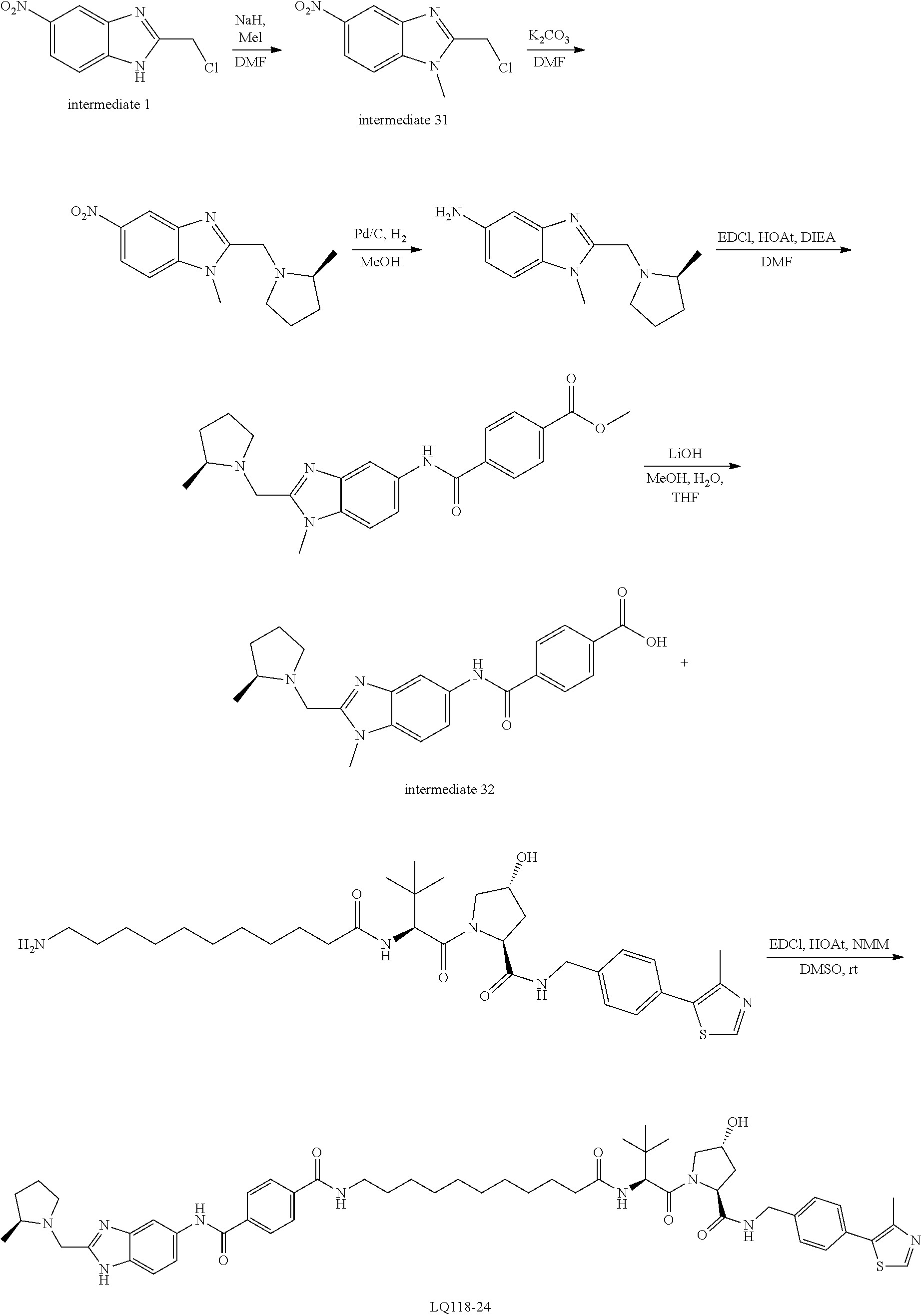

### Intermediate 31 2-(chloromethyl)-1-methyl-5-nitro-1H-benzo[d]imidazole

Sodium hydride (60 mg, 1.5 mmol, 60% in mineral oil) was added in portions to a solution of intermediate 1 (211 mg, 1 mmol) in dry dimethylformamide (3 mL) at ice bath. The mixture was stirred for 30 min at the same temperature, then iodomethane (63 μL, 1 mmol) was added. The resultant mixture was stirred for 1 h at room temperature. After cooling with ice bath, water was added slowly to quench the excess of sodium hydride. The mixture was extracted with ethyl acetate. Combined organic phases was washed with water and brine, dried over anhydrous sodium sulfate and concentrated. The residue was purified to by silica gel flash chromatography yield the title compound as yellow solid (136 mg, 61%). MS (ESI): m/z 226.1 [M+H]⁺.

### Intermediate 32 (S)-4-((1-methyl-2-((2-methylpyrrolidin-1-yl)methyl)-1H-benzo[d]imidazol-5-yl)carbamoyl)benzoic acid

Intermediate 32 was synthesized according to the procedures for the preparation of intermediate 10 as a white solid in 69% yield. ¹H NMR (600 MHz, Methanol-d₄) δ 8.15 (d, J=1.9 Hz, 1H), 8.05-8.00 (m, 2H), 7.95-7.89 (m, 2H), 7.55 (dd, J=8.8, 2.0 Hz, 1H), 7.50 (d, J=8.8 Hz, 1H), 4.84 (d, J=15.5 Hz, 1H), 4.58 (d, J=15.4 Hz, 1H), 3.83 (s, 3H), 3.79-3.68 (m, 2H), 3.38-3.30 (m, 1H), 2.35-2.26 (m, 1H), 2.12-1.97 (m, 2H), 1.79-1.71 (m, 1H), 1.43 (d, J=6.6 Hz, 3H). MS (ESI): m/z 393.1 [M+H]⁺.

LQ118-24 was synthesized following the standard procedure for preparing LQ076-105 from intermediate 32 (10 mg, 0.02 mmol), (2S,4R)-1-((S)-2-(11-aminoundecanamido)-3,3-dimethylbutanoyl)-4-hydroxy-N-(4-(4-methylthiazol-5-yl)benzyl)pyrrolidine-2-carboxamide (15.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ118-24 was obtained as white solid in TFA salt form (16.3 mg, 65%). ¹H NMR (600 MHz, Methanol-d₄) δ 9.01 (s, 1H), 8.26 (dd, J=7.8, 1.9 Hz, 1H), 8.09-8.03 (m, 2H), 8.00-7.94 (m, 2H), 7.71 (d, J=8.7 Hz, 1H), 7.67-7.60 (m, 1H), 7.52-7.42 (m, 4H), 4.97-4.91 (m, 3H), 4.70-4.63 (m, 2H), 4.62-4.49 (m, 3H), 4.37 (d, J=15.5 Hz, 1H), 3.96-3.86 (m, 6H), 3.82 (dd, J=10.9, 3.9 Hz, 1H), 3.49-3.39 (m, 4H), 2.50 (s, 3H), 2.46-2.39 (m, 1H), 2.35-2.07 (m, 5H), 1.91-1.83 (m, 1H), 1.70-1.58 (m, 4H), 1.54 (dd, J=6.6, 2.3 Hz, 3H), 1.46-1.32 (m, 12H), 1.05 (s, 9H). HRMS m/z [M+H]⁺ calcd for C₅₅H₇₄N₉O₆S⁺ 988.5477, found 988.5465.

### Example 180

### Synthesis of LQ118-25

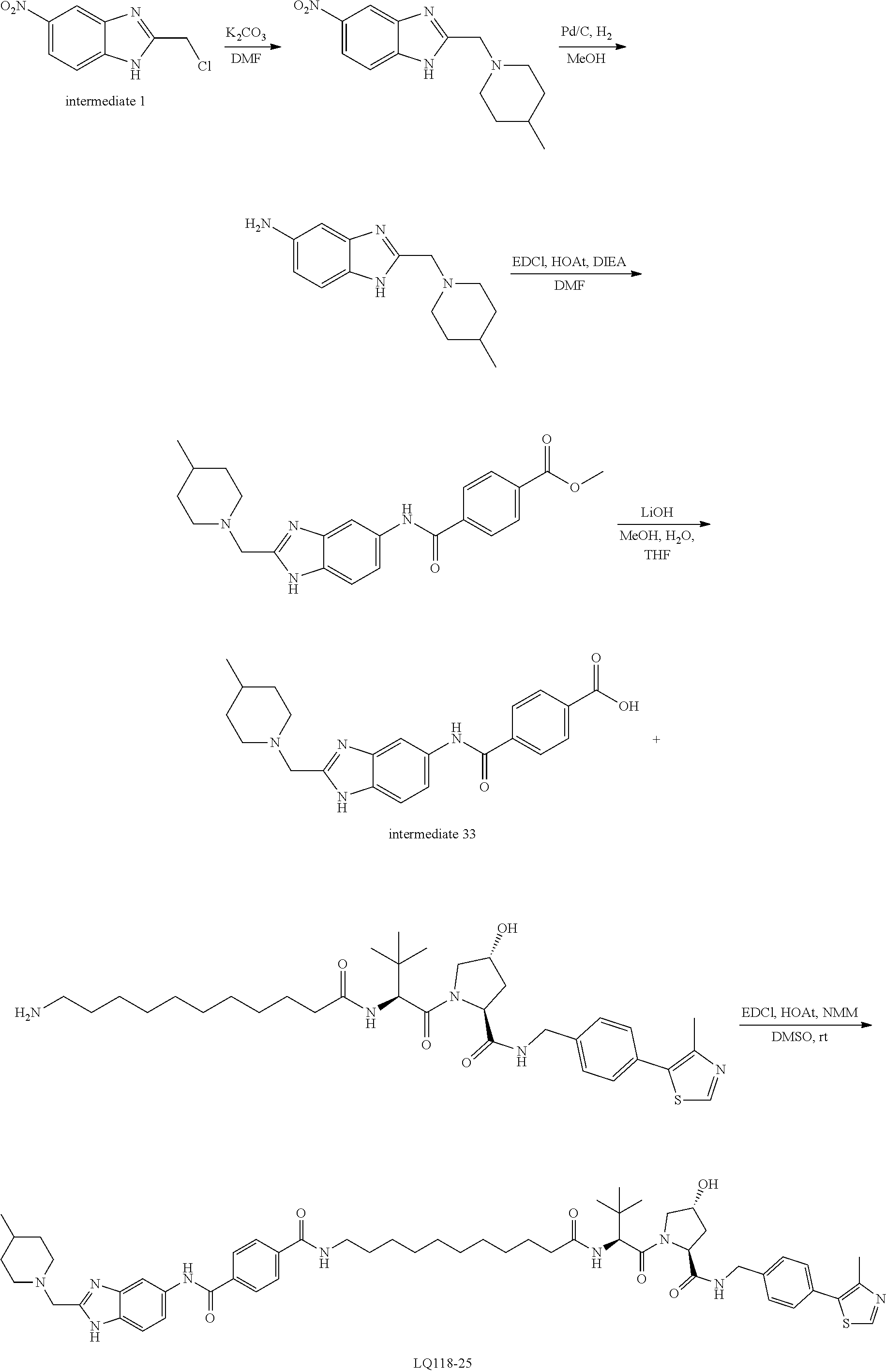

### Intermediate 33 4-((2-((4-methylpiperidin-1-yl)methyl)-1H-benzo[d]imidazol-5-yl)carbamoyl)benzoic acid

Intermediate 33 was synthesized according to the procedures for the preparation of Intermediate 10 as a white solid in 78% yield. ¹H NMR (600 MHz, Methanol-d₄) δ 9.53 (d, J=1.7 Hz, 1H), 8.56 (s, 1H), 8.20 (dd, J=9.3, 1.7 Hz, 1H), 7.93 (dd, J=9.8, 2.8 Hz, 1H), 7.86-7.82 (m, 1H), 7.37-7.31 (m, 5H), 7.08 (d, J=9.8 Hz, 1H), 4.54 (s, 2H), 3.46 (t, J=7.3 Hz, 2H), 3.43-3.39 (m, 5H), 3.11 (t, J=7.2 Hz, 2H). MS (ESI): m/z 393.4 [M+H]⁺.

LQ118-25 was synthesized following the standard procedure for preparing LQ076-105 from intermediate 33 (10 mg, 0.02 mmol), (2S,4R)-1-((S)-2-(11-aminoundecanamido)-3,3-dimethylbutanoyl)-4-hydroxy-N-(4-(4-methylthiazol-5-yl)benzyl)pyrrolidine-2-carboxamide (15.3 mg, 0.02 mmol, 1.0 equiv), EDCI (5.8 mg, 0.03 mmol, 1.5 equiv), HOAt (4.1 mg, 0.03 mmol, 1.5 equiv), and NMM (6.1 mg, 0.06 mmol, 3.0 equiv) in DMSO (1 mL). LQ118-25 was obtained as white solid in TFA salt form (16.3 mg, 65%). ¹H NMR (600 MHz, Methanol-d₄) δ 9.05 (s, 1H), 8.33 (d, J=2.0 Hz, 1H), 8.07-8.04 (m, 2H), 7.99-7.95 (m, 2H), 7.70 (d, J=8.5 Hz, 1H), 7.59 (dd, J=8.8, 2.0 Hz, 1H), 7.51-7.43 (m, 4H), 4.65 (s, 1H), 4.62-4.50 (m, 5H), 4.38 (d, J=15.5 Hz, 1H), 3.94-3.90 (m, 1H), 3.82 (dd, J=11.0, 3.9 Hz, 1H), 3.69-3.64 (m, 2H), 3.42 (t, J=7.2 Hz, 2H), 3.20-3.13 (m, 2H), 2.50 (s, 3H), 2.35-2.21 (m, 3H), 2.12-2.07 (m, 1H), 2.00-1.95 (m, 2H), 1.79-1.71 (m, 1H), 1.70-1.50 (m, 5H), 1.46-1.31 (m, 16H), 1.05 (s, 9H). HRMS m/z [M+H]⁺ calcd for C₅₅H₇₄N₉O₆S⁺ 988.5477, found 988.5471.

### Example 181

### Synthesis of Intermediate 40

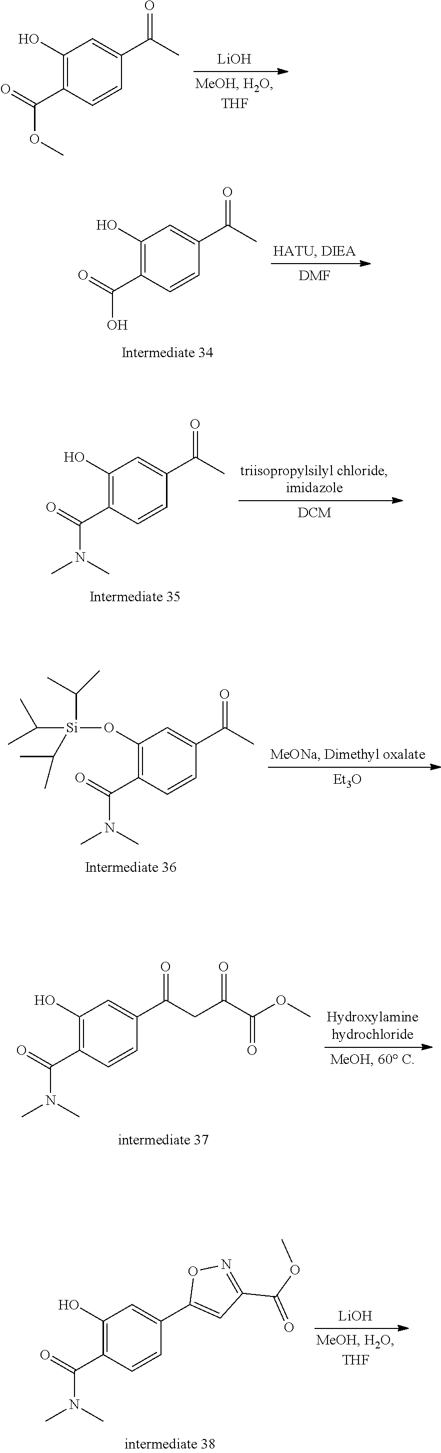

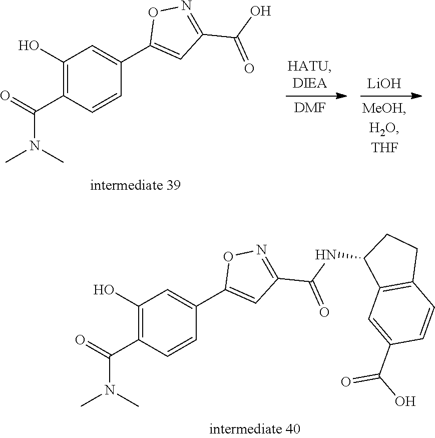

### Intermediate 34: 4-acetyl-2-hydroxybenzoic acid

Intermediate 34 was synthesized according to the procedures for the preparation of intermediate 4 as a white solid in 78% yield. ¹H NMR (600 MHz, Methanol-d₄) δ 7.97 (d, J=8.1 Hz, 1H), 7.50-7.45 (m, 2H), 2.60 (s, 3H). MS (ESI): m/z 179.0 [M−H]⁺.

### Intermediate 35: 4-acetyl-2-hydroxy-N,N-dimethylbenzamide

Intermediate 35 was synthesized according to the procedures for the preparation of intermediate 3 as a white solid in 76%. ¹H NMR (600 MHz, Methanol-d₄) δ 7.57-7.51 (m, 1H), 7.45 (s, 1H), 7.33-7.28 (m, 1H), 3.02 (d, J=103.3 Hz, 6H), 2.58 (s, 1H). MS (ESI): m/z 208.3 [M+H]⁺.

### Intermediate 36: 4-acetyl-N,N-dimethyl-2-((triisopropylsilyl)oxy)benzamide

To a solution of intermediate 35 (400 mg, 1.93 mmol) was added imidazole (263 mg, 3.86 mmol) and triisopropylsilyl chloride (667 mg, 3.47 mmol). The resulting mixture was stirred 6 h at RT.

After the reaction was completed, the reaction mixture was poured into water, aqueous phase was extracted with DCM. The combined organic phase was washed with brine, dried and concentrated. The resulting residue was purified by silica gel flash chromatography to give the compound as yellow oil (525 mg, 75%). ¹H NMR (600 MHz, Chloroform-d) δ 7.54 (dd, J=7.8, 1.5 Hz, 1H), 7.43 (d, J=1.5 Hz, 1H), 7.32 (d, J=7.8 Hz, 1H), 3.08 (s, 3H), 2.86 (s, 3H), 2.57 (s, 3H), 1.35-1.27 (m, 3H), 1.13-1.03 (m, 18H). MS (ESI): m/z 364.5 [M+H]⁺.

### Intermediate 37: Methyl 4-(4-(dimethylcarbamoyl)-3-hydroxyphenyl)-2,4-dioxobutanoate

A solution of intermediate 36 (560 mg, 1.54 mmol) and dimethyl oxalate (182 mg, 1.54 mmol) in Et₂O was added sodium methoxide solution (0.5 M, 4 mL) slowly. The resulting mixture was stirred overnight at RT. After the reaction was completed, the mixture was purified by reverse phase C18 column (10%-100% acetonitrile/0.1% TFA in water) to afford intermediate 37 as white solid (85 mg, 19%). ¹H NMR (600 MHz, Methanol-d₄) δ 7.58 (d, J=7.8 Hz, 1H), 7.51 (s, 1H), 7.34 (d, J=7.8 Hz, 1H), 7.07 (s, 1H), 3.92 (s, 3H), 3.11 (s, 3H), 2.94 (s, 3H). MS (ESI): m/z 294.2 [M+H]⁺.

### Intermediate 38: Methyl 5-(4-(dimethylcarbamoyl)-3-hydroxyphenyl)isoxazole-3-carboxylate

The intermediate 37 (97 mg, 0.33 mmol) was dissolved in methanol and treated with hydroxylamine hydrochloride (69 mg, 1 mmol) The resulting mixture was heated to 55° C. overnight. Then the mixture was purified by reverse phase C18 column (10%-100% methanol/0.1% TFA in water) to afford intermediate 38 as white solid (57 mg, 60%). ¹H NMR (600 MHz, Methanol-d₄) δ 7.43 (dd, J=7.9, 1.5 Hz, 1H), 7.36 (d, J=1.5 Hz, 1H), 7.34 (d, J=7.9 Hz, 1H), 7.18 (s, 1H), 3.98 (s, 3H), 3.04 (d, J=79.3 Hz, 6H). MS (ESI): m/z 291.3 [M+H]⁺.

### Intermediate 39: 5-(4-(dimethylcarbamoyl)-3-hydroxyphenyl)isoxazole-3-carboxylic acid

Intermediate 39 was synthesized according to the procedures for the preparation of intermediate 4 as a white solid in 34% yield. ¹H NMR (600 MHz, Methanol-d₄) δ 7.48-7.41 (m, 1H), 7.40-7.32 (m, 2H), 7.19-7.13 (m, 1H), 3.06 (d, J=76.5 Hz, 6H). MS (ESI): m/z 277.1 [M+H]⁺.

### Intermediate 40: (R)-3-(5-(4-(dimethylcarbamoyl)-3-hydroxyphenyl)isoxazole-3-carboxamido)-2,3-dihydro-1H-indene-5-carboxylic acid

Intermediate 40 was synthesized according to the procedures for the preparation of intermediate 4 as a white solid in 66% yield. ¹H NMR (600 MHz, Methanol-d₄) δ 7.98 (s, 1H), 7.94 (dd, J=7.9, 1.6 Hz, 1H), 7.44 (dd, J=7.9, 1.6 Hz, 1H), 7.40-7.36 (m, 2H), 7.35 (d, J=7.9 Hz, 1H), 7.16 (s, 1H), 5.70 (t, J=7.7 Hz, 1H), 3.37 (s, 1H), 3.18-2.94 (m, 8H), 2.70-2.63 (m, 1H), 2.18-2.11 (m, 1H). MS (ESI): m/z 435.9 [M+H]⁺.

### Example 182

### Synthesis of LQ108-58

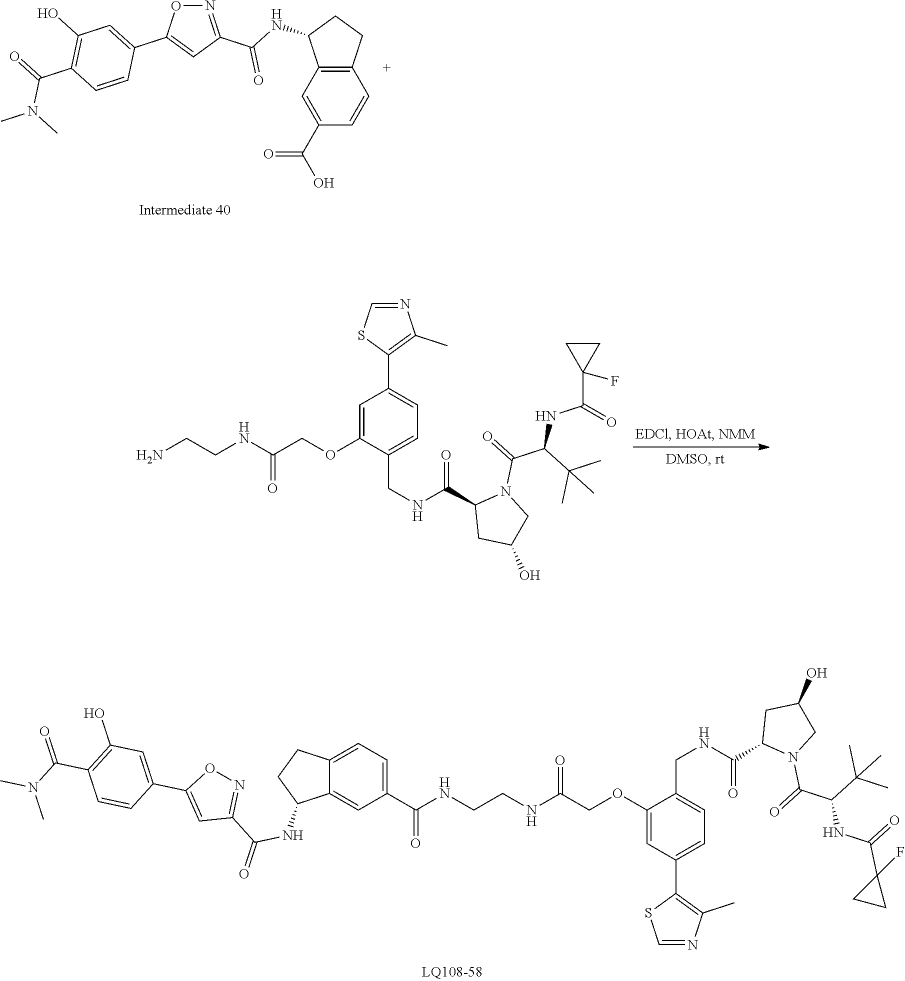 [SMILES: CN(C)C(=O)c1ccc(-c2cc(C(=O)N[C@@H]3CCc4ccc(C(=O)O)cc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCNC(=O)c2ccc3c(c2)[C@H](NC(=O)c2cc(-c4ccc(C(=O)N(C)C)c(O)c4)on2)CC3)c1]

To a solution of Intermediate 40 (5 mg, 0.01 mmol) in DMSO (1 mL) were added (2S,4R)—N-(2-(2-((2-aminoethyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (7.5 mg, 0.01 mmol, 1.0 equiv), EDCI (1-ethyl-3-(3-dimethylaminopropyl)carbodiimide) (2.9 mg, 0.015 mmol, 1.5 equiv), HOAt (1-hydroxy-7-azabenzo-triazole) (2.1 mg, 0.015 mmol, 1.5 equiv), and NMM (N-Methylmorpholine) (3.1 mg, 0.03 mmol, 3.0 equiv). After being stirred overnight at room temperature, the resulting mixture was purified by preparative HPLC (5%-70% acetonitrile/0.1% TFA in H₂O) to afford LQ108-58 as white solid (8 mg, 76%). ¹H NMR (600 MHz, Methanol-d₄) δ 9.12 (s, 1H), 7.75 (s, 1H), 7.70 (dd, J=8.0, 1.7 Hz, 1H), 7.54-7.44 (m, 3H), 7.37-7.32 (m, 3H), 7.18 (s, 1H), 6.96 (dd, J=5.4, 1.6 Hz, 2H), 5.69 (t, J=7.9 Hz, 1H), 4.75-4.71 (m, 1H), 4.66-4.43 (m, 6H), 3.87-3.75 (m, 2H), 3.60-3.48 (m, 4H), 3.18-2.93 (m, 8H), 2.70-2.63 (m, 1H), 2.46 (s, 3H), 2.21-2.10 (m, 2H), 2.09-2.00 (m, 2H), 1.41-1.23 (m, 4H), 1.02 (s, 9H). HRMS m/z [M+H]⁺ calcd for C₅₃H₆₁FN₉O₁₁S⁺ 1050.4190, found 1050.4182.

### Example 183

### Synthesis of LQ108-60

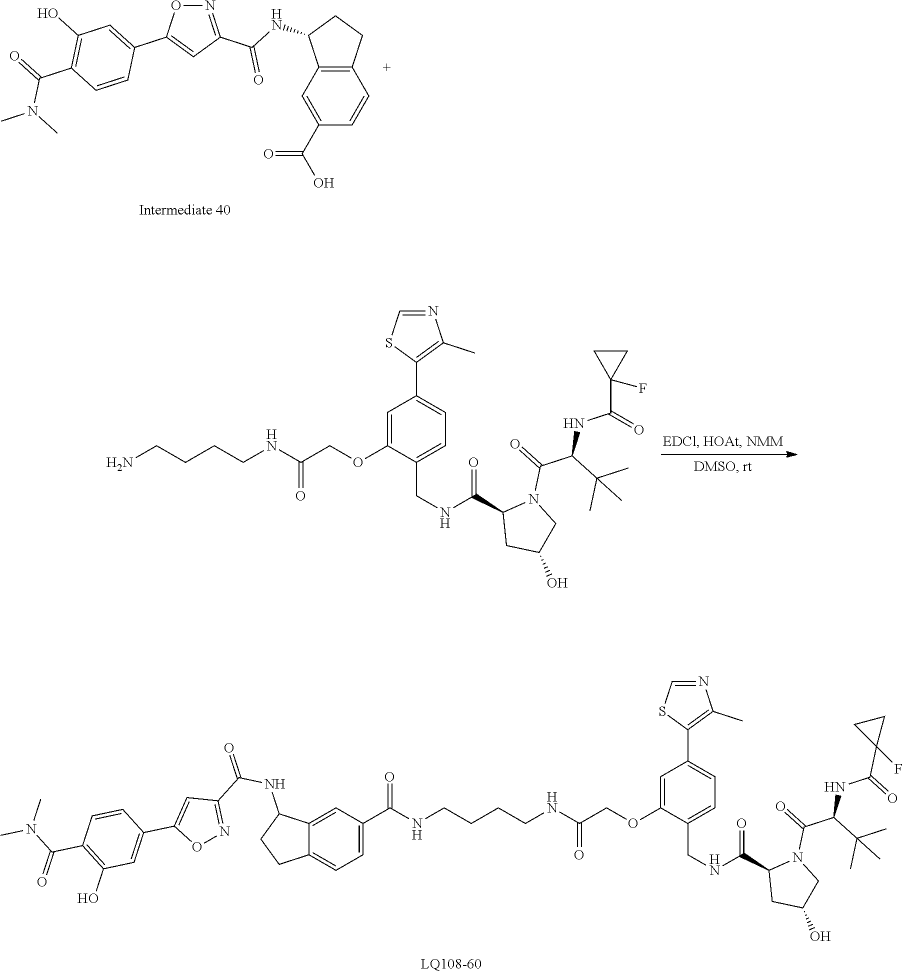 [SMILES: CN(C)C(=O)c1ccc(-c2cc(C(=O)N[C@@H]3CCc4ccc(C(=O)O)cc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCNC(=O)c2ccc3c(c2)C(NC(=O)c2cc(-c4ccc(C(=O)N(C)C)c(O)c4)on2)CC3)c1]

LQ108-60 was synthesized following the standard procedure for preparing LQ108-58 from intermediate 40 (5 mg, 0.01 mmol), (2S,4R)—N-(2-(2-((4-aminobutyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (7.8 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-60 was obtained as white solid (8.4 mg, 78%). ¹H NMR (600 MHz, Methanol-d₄) δ 8.98 (s, 1H), 7.78 (s, 1H), 7.73 (dd, J=7.9, 1.7 Hz, 1H), 7.47-7.39 (m, 3H), 7.38-7.30 (m, 3H), 7.15 (s, 1H), 7.06 (dd, J=7.7, 1.6 Hz, 1H), 6.95 (d, J=1.6 Hz, 1H), 5.69 (t, J=7.9 Hz, 1H), 4.71-4.67 (m, 1H), 4.63-4.53 (m, 4H), 4.47-4.40 (m, 2H), 3.78 (d, J=11.1 Hz, 1H), 3.73 (dd, J=11.1, 3.8 Hz, 1H), 3.37-3.32 (m, 2H), 3.17-2.93 (m, 8H), 2.69-2.61 (m, 1H), 2.47 (s, 3H), 2.19-2.09 (m, 2H), 2.05-1.99 (m, 1H), 1.65-1.57 (m, 4H), 1.38-1.22 (m, 6H), 0.98 (s, 9H). HRMS m/z [M+H]⁺ calcd for C₅₅H₆₅FN₉O₁₁S⁺ 1078.4503, found 1078.4501.

### Example 184

### Synthesis of LQ108-61

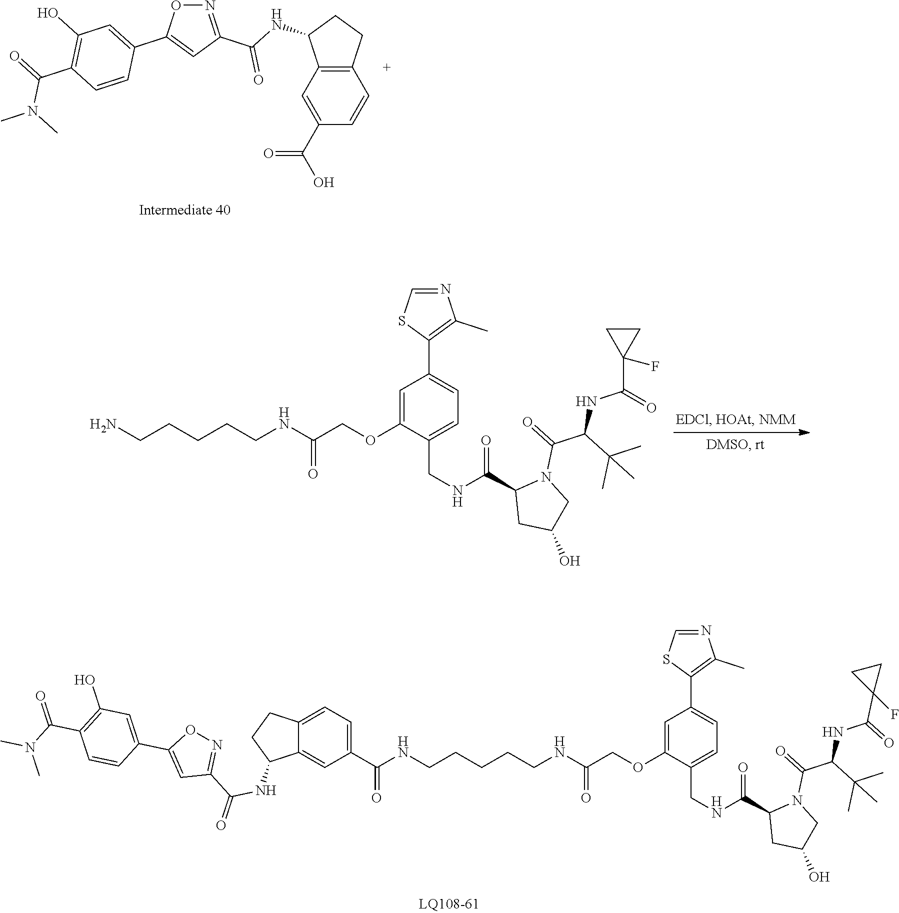 [SMILES: CN(C)C(=O)c1ccc(-c2cc(C(=O)N[C@@H]3CCc4ccc(C(=O)O)cc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCNC(=O)c2ccc3c(c2)[C@H](NC(=O)c2cc(-c4ccc(C(=O)N(C)C)c(O)c4)on2)CC3)c1]

LQ108-61 was synthesized following the standard procedure for preparing LQ108-58 from intermediate 40 (5 mg, 0.01 mmol), (2S,4R)—N-(2-(2-((5-aminopentyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (7.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-61 was obtained as white solid (7.6 mg, 70%). ¹H NMR (600 MHz, Methanol-d₄) δ 9.02 (s, 1H), 7.80 (s, 1H), 7.74 (dd, J=7.8, 1.7 Hz, 1H), 7.50-7.41 (m, 3H), 7.38-7.33 (m, 3H), 7.16 (s, 1H), 7.08 (dd, J=7.7, 1.6 Hz, 1H), 6.96 (d, J=1.6 Hz, 1H), 5.69 (t, J=7.9 Hz, 1H), 4.74-4.71 (m, 1H), 4.64-4.52 (m, 4H), 4.48 (dd, J=4.8, 2.5 Hz, 1H), 4.43 (d, J=15.0 Hz, 1H), 3.84 (d, J=10.9 Hz, 1H), 3.77 (dd, J=11.0, 3.8 Hz, 1H), 3.40-3.34 (m, 2H), 3.18-2.94 (m, 8H), 2.68-2.62 (m, 1H), 2.50 (s, 3H), 2.23-2.03 (m, 3H), 1.65-1.57 (m, 4H), 1.42-1.24 (m, 8H), 1.00 (s, 9H). HRMS m/z [M+H]⁺ calcd for C₅₆H₆₇FN₉O₁₁S⁺ 1092.4659, found 1092.4648.

### Example 185

### Synthesis of LQ108-62

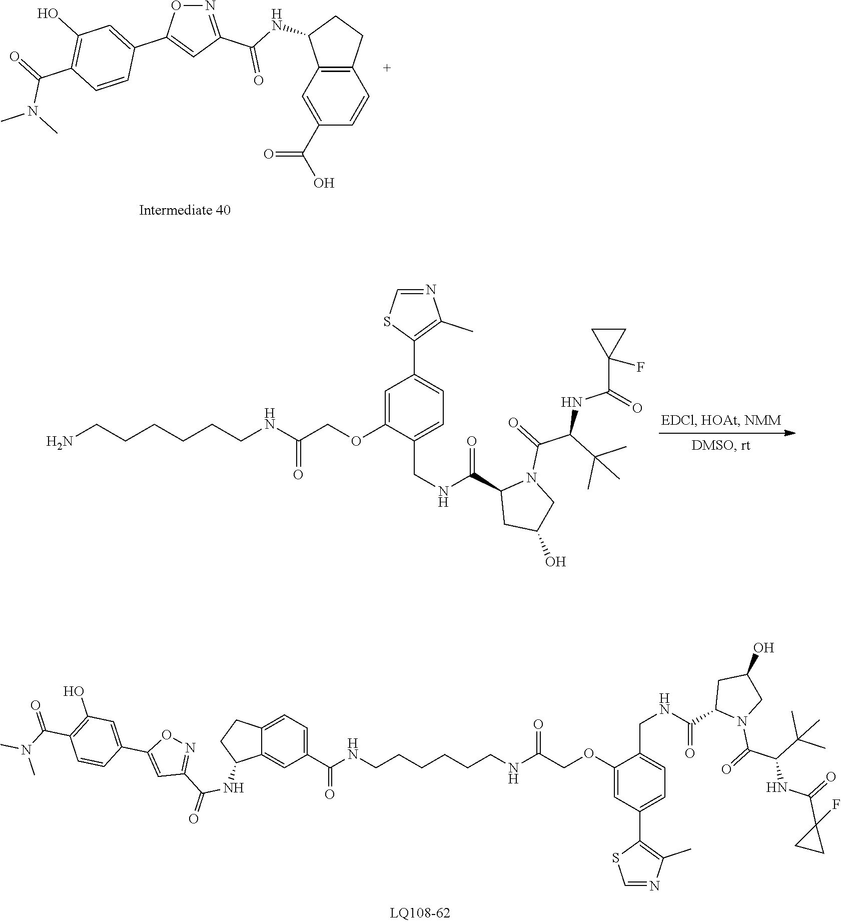 [SMILES: CN(C)C(=O)c1ccc(-c2cc(C(=O)N[C@@H]3CCc4ccc(C(=O)O)cc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCNC(=O)c2ccc3c(c2)[C@H](NC(=O)c2cc(-c4ccc(C(=O)N(C)C)c(O)c4)on2)CC3)c1]

LQ108-62 was synthesized following the standard procedure for preparing LQ108-58 from intermediate 40 (5 mg, 0.01 mmol), (2S,4R)—N-(2-(2-((6-aminohexyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (8.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-62 was obtained as white solid (7.9 mg, 71%). ¹H NMR (600 MHz, Methanol-d₄) δ 9.02 (s, 1H), 7.79 (s, 1H), 7.73 (dd, J=7.9, 1.7 Hz, 1H), 7.49-7.43 (m, 2H), 7.40 (dd, J=7.9, 1.6 Hz, 1H), 7.38-7.31 (m, 3H), 7.14 (s, 1H), 7.07 (dd, J=7.7, 1.6 Hz, 1H), 6.95 (d, J=1.6 Hz, 1H), 5.69 (t, J=7.9 Hz, 1H), 4.72-4.69 (m, 1H), 4.62-4.53 (m, 4H), 4.48-4.42 (m, 2H), 3.82 (d, J=11.1 Hz, 1H), 3.75 (dd, J=11.1, 3.8 Hz, 1H), 3.36-3.32 (m, 2H), 3.27 (t, J=7.0 Hz, 2H), 3.16-2.91 (m, 8H), 2.68-2.61 (m, 1H), 2.48 (s, 3H), 2.21-2.16 (m, 1H), 2.14-2.09 (m, 1H), 2.07-2.01 (m, 1H), 1.57-1.50 (m, 4H), 1.40-1.23 (m, 8H), 0.99 (s, 9H). HRMS m/z [M+H]⁺ calcd for C₅₇H₆₉FN₉O₁₁S⁺ 1106.4816, found 1106.4813.

### Example 186

### Synthesis of LQ108-63

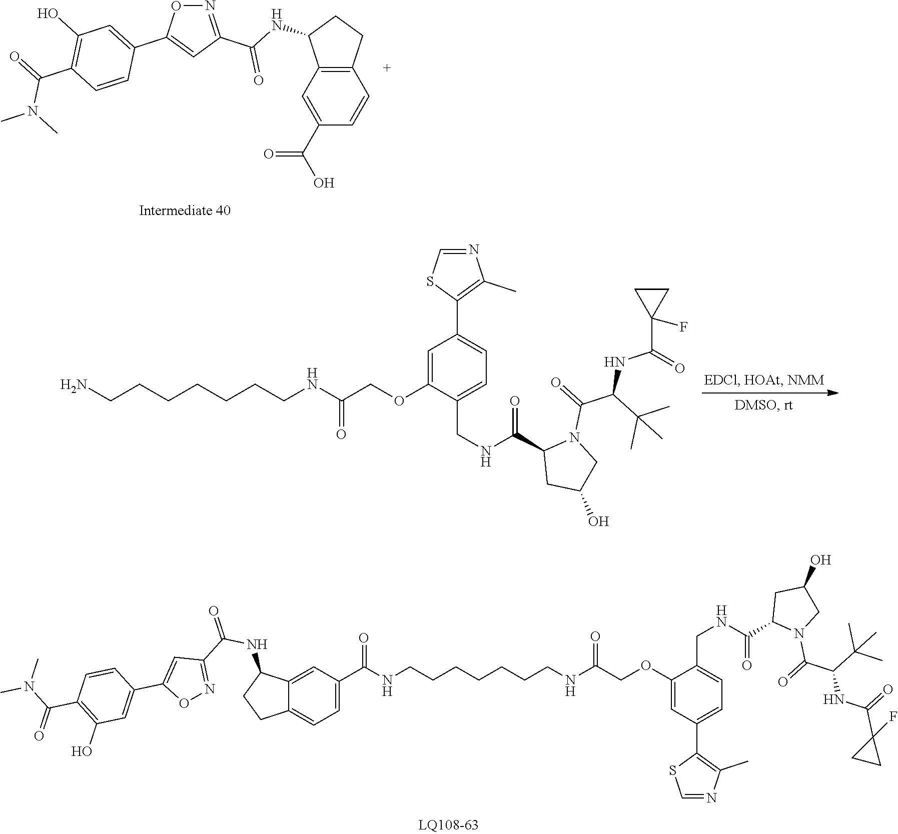 [SMILES: CN(C)C(=O)c1ccc(-c2cc(C(=O)N[C@@H]3CCc4ccc(C(=O)O)cc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCCNC(=O)c2ccc3c(c2)[C@H](NC(=O)c2cc(-c4ccc(C(=O)N(C)C)c(O)c4)on2)CC3)c1]

LQ108-63 was synthesized following the standard procedure for preparing LQ108-58 from intermediate 40 (5 mg, 0.01 mmol), (2S,4R)—N-(2-(2-((7-aminoheptyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (8.2 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-63 was obtained as white solid (7.3 mg, 65%). ¹H NMR (600 MHz, Methanol-d₄) δ 9.07 (s, 1H), 7.80 (s, 1H), 7.74 (dd, J=8.0, 1.7 Hz, 1H), 7.50-7.46 (m, 2H), 7.43 (dd, J=7.9, 1.6 Hz, 1H), 7.40-7.33 (m, 3H), 7.16 (s, 1H), 7.09 (dd, J=7.8, 1.6 Hz, 1H), 6.97 (d, J=1.6 Hz, 1H), 5.70 (t, J=7.9 Hz, 1H), 4.76-4.71 (m, 1H), 4.64-4.54 (m, 4H), 4.52-4.45 (m, 2H), 3.85 (d, J=11.1 Hz, 1H), 3.79 (dd, J=11.0, 3.8 Hz, 1H), 3.37-3.34 (m, 2H), 3.30-3.26 (m, 2H), 3.18-2.94 (m, 8H), 2.70-2.63 (m, 1H), 2.50 (s, 3H), 2.24-2.19 (m, 1H), 2.17-2.04 (m, 2H), 1.62-1.51 (m, 4H), 1.41-1.24 (m, 10H), 1.02 (s, 9H). HRMS m/z [M+H]⁺ calcd for C₅₈H₇₁FN₉O₁₁S⁺ 1120.4972, found 1120.4970.

### Example 187

### Synthesis of LQ108-64

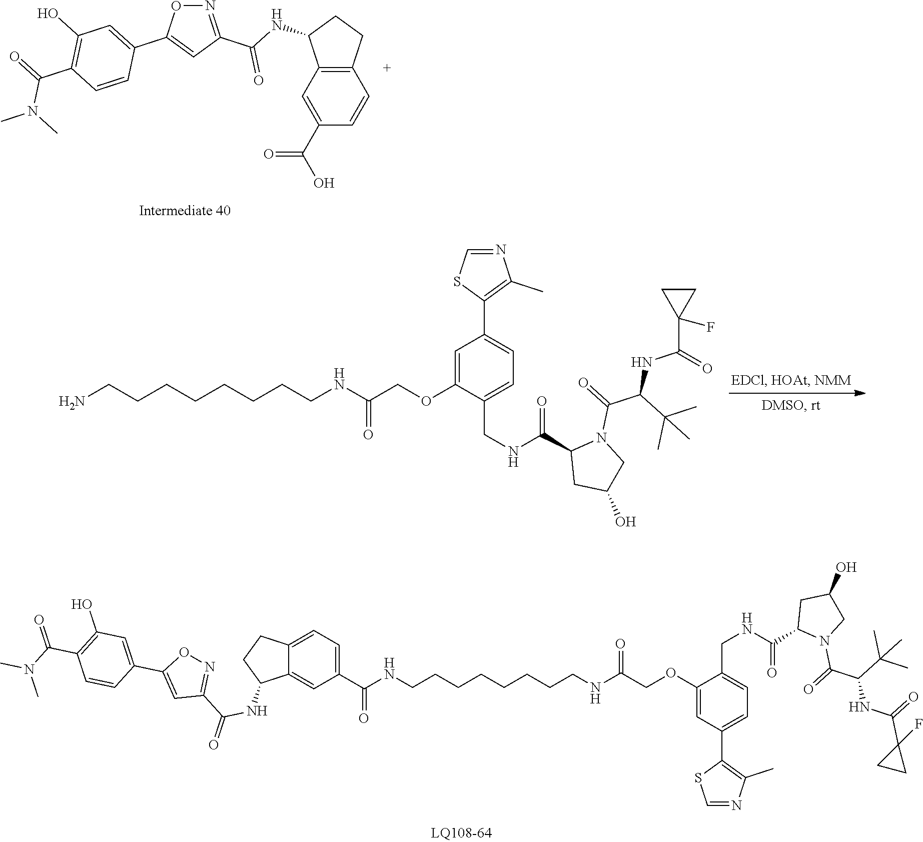 [SMILES: CN(C)C(=O)c1ccc(-c2cc(C(=O)N[C@@H]3CCc4ccc(C(=O)O)cc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCCCNC(=O)c2ccc3c(c2)[C@H](NC(=O)c2cc(-c4ccc(C(=O)N(C)C)c(O)c4)on2)CC3)c1]

LQ108-64 was synthesized following the standard procedure for preparing LQ108-58 from intermediate 40 (5 mg, 0.01 mmol), (2S,4R)—N-(2-(2-((8-aminooctyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (8.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-64 was obtained as white solid (7.9 mg, 70%). ¹H NMR (600 MHz, Methanol-d₄) δ 9.01 (s, 1H), 7.78 (s, 1H), 7.73 (dd, J=8.0, 1.7 Hz, 1H), 7.50-7.45 (m, 2H), 7.41 (dd, J=7.9, 1.6 Hz, 1H), 7.38-7.31 (m, 3H), 7.14 (s, 1H), 7.08 (dd, J=7.8, 1.6 Hz, 1H), 6.95 (d, J=1.6 Hz, 1H), 5.69 (t, J=7.9 Hz, 1H), 4.74-4.70 (m, 1H), 4.62-4.53 (m, 4H), 4.50-4.43 (m, 2H), 3.83 (d, J=10.8 Hz, 1H), 3.77 (dd, J=11.1, 3.8 Hz, 1H), 3.36-3.32 (m, 2H), 3.25 (t, J=7.0 Hz, 2H), 3.16-2.92 (m, 8H), 2.68-2.61 (m, 1H), 2.48 (s, 3H), 2.23-2.17 (m, 1H), 2.15-2.02 (m, 2H), 1.60-1.47 (m, 4H), 1.39-1.22 (m, 12H), 1.00 (s, 9H). HRMS m/z [M+H]⁺ calcd for C₅₉H₇₃FN₉O₁₁S⁺ 1134.5129, found 1134.5119.

### Example 188

### Synthesis of LQ108-65

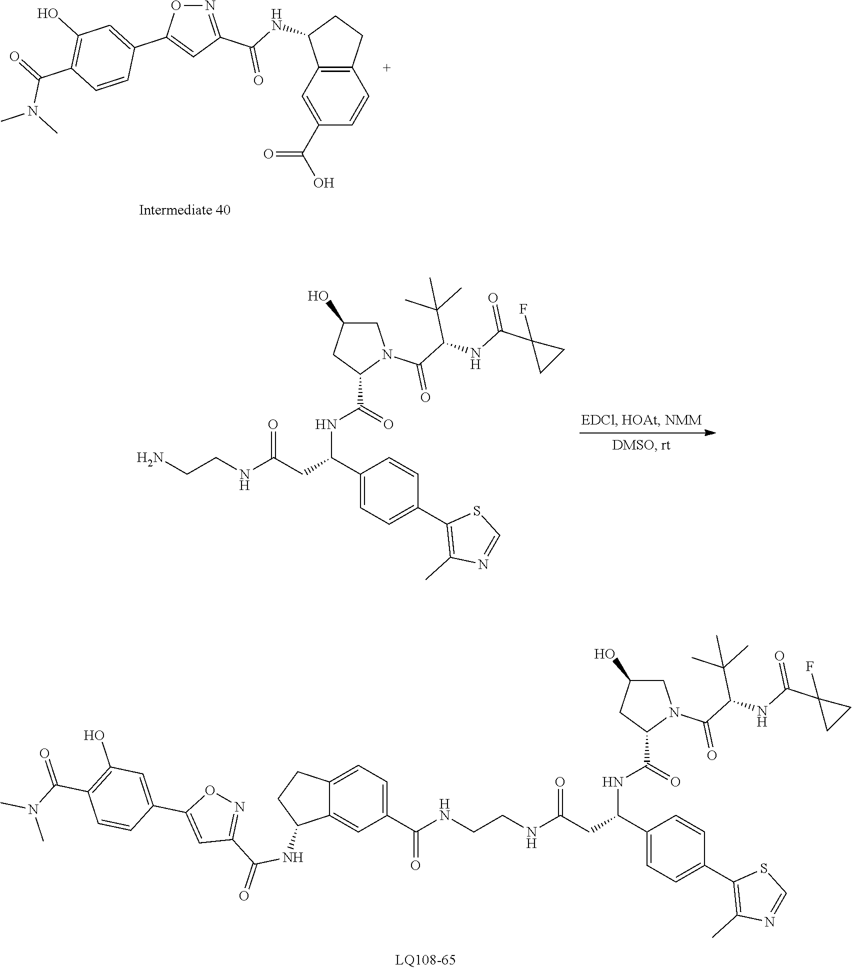 [SMILES: CN(C)C(=O)c1ccc(-c2cc(C(=O)N[C@@H]3CCc4ccc(C(=O)O)cc43)no2)cc1O ; Cc1ncsc1-c1ccc([C@H](CC(=O)NCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1]

LQ108-65 was synthesized following the standard procedure for preparing LQ108-58 from intermediate 40 (5 mg, 0.01 mmol), (2S,4R)—N—((S)-3-((2-aminoethyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (7.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-65 was obtained as white solid (6.2 mg, 60%).

¹H NMR (600 MHz, Methanol-d₄) δ 9.00 (s, 1H), 7.66 (s, 1H), 7.56 (dd, J=7.9, 1.7 Hz, 1H), 7.38-7.29 (m, 3H), 7.27-7.19 (m, 5H), 7.05 (s, 1H), 5.56 (t, J=7.9 Hz, 1H), 5.23 (t, J=7.0 Hz, 1H), 4.64-4.59 (m, 1H), 4.49 (dd, J=9.3, 7.6 Hz, 1H), 4.35-4.31 (m, 1H), 3.72 (d, J=11.2 Hz, 1H), 3.65 (dd, J=11.1, 3.8 Hz, 1H), 3.32-3.26 (m, 4H), 3.06-2.81 (m, 8H), 2.71 (dd, J=14.2, 6.8 Hz, 1H), 2.64 (dd, J=14.2, 7.3 Hz, 1H), 2.56-2.49 (m, 1H), 2.35 (s, 3H), 2.12-2.07 (m, 1H), 2.04-1.97 (m, 1H), 1.87-1.81 (m, 1H), 1.30-1.10 (m, 4H), 0.93 (s, 9H). HRMS m/z [M+H]+ calcd for C₅₃H₆₁FN₉O₁₀S⁺ 1034.4241, found 1034.4243.

### Example 189

### Synthesis of LQ108-66

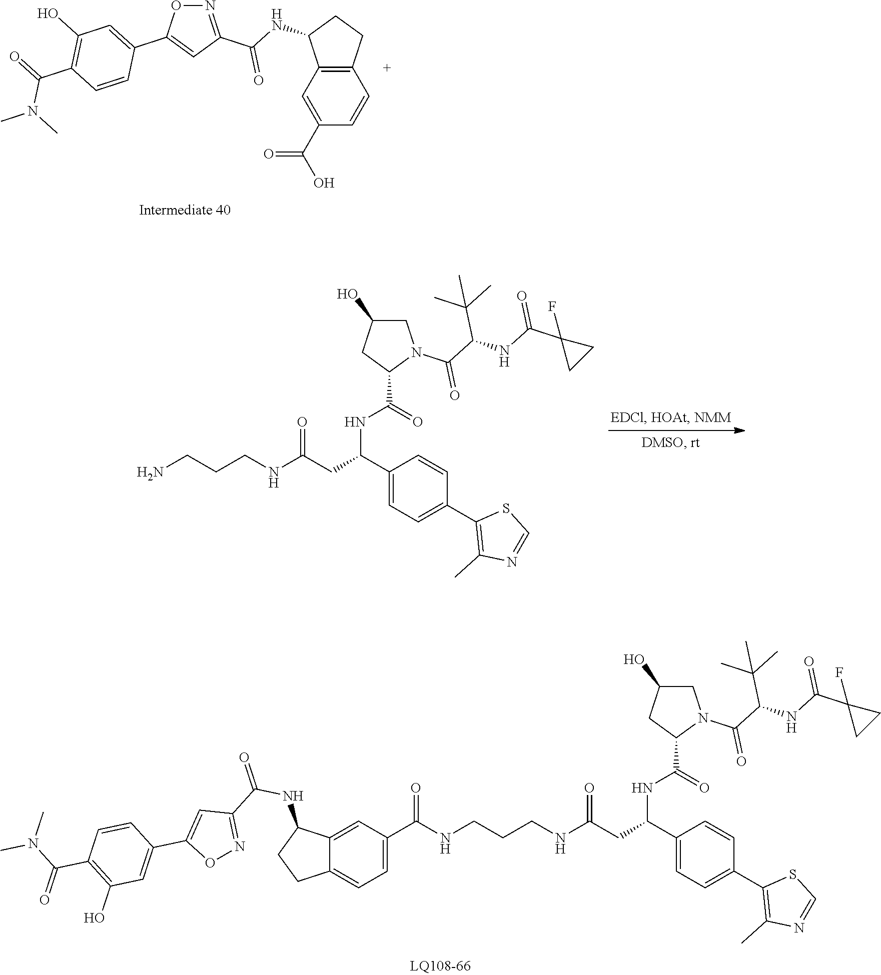 [SMILES: CN(C)C(=O)c1ccc(-c2cc(C(=O)N[C@@H]3CCc4ccc(C(=O)O)cc43)no2)cc1O ; Cc1ncsc1-c1ccc([C@H](CC(=O)NCCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1]

LQ108-66 was synthesized following the standard procedure for preparing LQ108-58 from intermediate 40 (5 mg, 0.01 mmol), (2S,4R)—N—((S)-3-((3-aminopropyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (7.5 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-66 was obtained as white solid (6.9 mg, 66%).

¹H NMR (600 MHz, Methanol-d₄) δ 9.06 (s, 1H), 7.77 (s, 1H), 7.70 (dd, J=8.0, 1.7 Hz, 1H), 7.48-7.45 (m, 2H), 7.44-7.39 (m, 3H), 7.37-7.31 (m, 2H), 7.18 (s, 1H), 5.70 (t, J=8.0 Hz, 1H), 5.33 (dd, J=8.1, 6.0 Hz, 1H), 4.75-4.70 (m, 1H), 4.63 (dd, J=9.2, 7.6 Hz, 1H), 4.45-4.41 (m, 1H), 3.82 (d, J=11.1 Hz, 1H), 3.76 (dd, J=11.1, 3.8 Hz, 1H), 3.25-3.18 (m, 1H), 3.17-2.91 (m, 11H), 2.85 (dd, J=14.1, 6.0 Hz, 1H), 2.75 (dd, J=14.1, 8.2 Hz, 1H), 2.69-2.62 (m, 1H), 2.44 (s, 3H), 2.25-2.09 (m, 2H), 1.98-1.92 (m, 1H), 1.64-1.56 (m, 2H), 1.40-1.23 (m, 4H), 1.05 (s, 9H). HRMS m/z [M+H]⁺ calcd for C₅₄H₆₃FN₉O₁₀S⁺ 1048.4397, found 1048.4402.

### Example 190

### Synthesis of LQ108-67

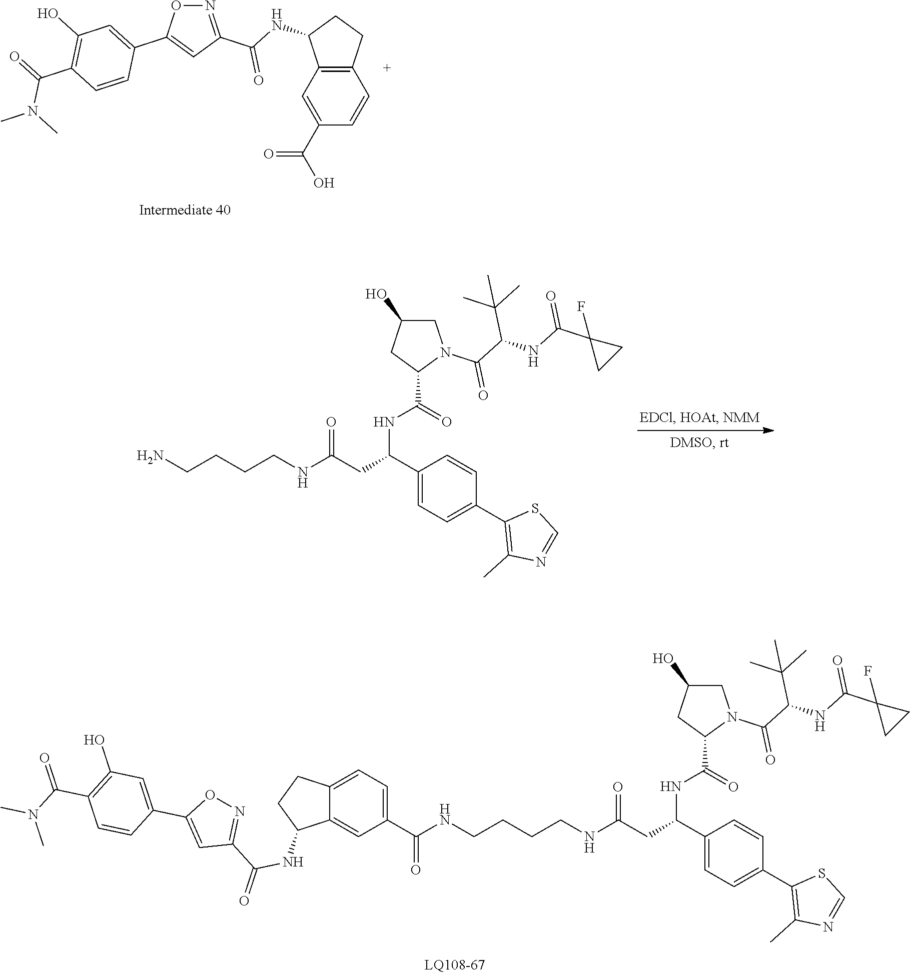 [SMILES: CN(C)C(=O)c1ccc(-c2cc(C(=O)N[C@@H]3CCc4ccc(C(=O)O)cc43)no2)cc1O ; Cc1ncsc1-c1ccc([C@H](CC(=O)NCCCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1]

LQ108-67 was synthesized following the standard procedure for preparing LQ108-58 from intermediate 40 (5 mg, 0.01 mmol), (2S,4R)—N—((S)-3-((4-aminobutyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (7.6 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-67 was obtained as white solid (7.3 mg, 69%).

¹H NMR (600 MHz, Methanol-d₄) δ 9.03 (s, 1H), 7.75 (s, 1H), 7.69 (dd, J=7.9, 1.7 Hz, 1H), 7.50-7.38 (m, 5H), 7.37-7.32 (m, 3H), 7.15 (s, 1H), 5.68 (t, J=7.9 Hz, 1H), 5.30 (dd, J=8.3, 6.1 Hz, 1H), 4.74-4.71 (m, 1H), 4.60-4.55 (m, 1H), 4.45-4.41 (m, 1H), 3.82 (d, J=11.1 Hz, 1H), 3.75 (dd, J=11.1, 3.8 Hz, 1H), 3.27 (t, J=6.7 Hz, 2H), 3.19-2.92 (m, 11H), 2.82 (dd, J=14.1, 6.1 Hz, 1H), 2.73 (dd, J=14.1, 8.4 Hz, 1H), 2.68-2.61 (m, 1H), 2.44 (s, 3H), 2.21-2.09 (m, 2H), 1.98-1.92 (m, 1H), 1.47-1.23 (m, 7H), 1.05 (s, 9H). HRMS m/z [M+H]⁺ calcd for C₅₅H₆₅FN₉O₁₀S⁺ 1062.4554, found 1062.4547.

### Example 191

### Synthesis of LQ108-68

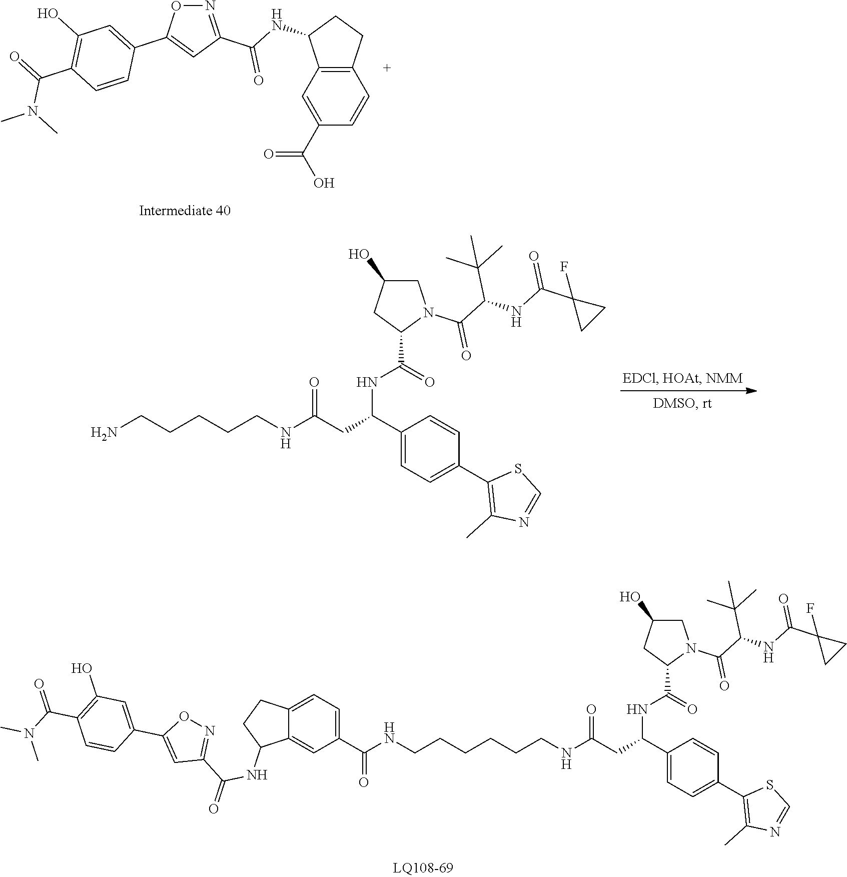 [SMILES: CN(C)C(=O)c1ccc(-c2cc(C(=O)N[C@@H]3CCc4ccc(C(=O)O)cc43)no2)cc1O ; Cc1ncsc1-c1ccc([C@H](CC(=O)NCCCCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCCCNC(=O)c2ccc3c(c2)C(NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1]

LQ108-68 was synthesized following the standard procedure for preparing LQ108-58 from intermediate 40 (5 mg, 0.01 mmol), (2S,4R)—N—((S)-3-((5-aminopentyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (7.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-68 was obtained as white solid (8.1 mg, 75%).

¹H NMR (600 MHz, Methanol-d₄) δ 8.88 (s, 1H), 7.67 (s, 1H), 7.62 (dd, J=7.9, 1.7 Hz, 1H), 7.37 (dd, J=9.4, 3.4 Hz, 1H), 7.34-7.21 (m, 7H), 7.06 (s, 1H), 5.59 (t, J=8.0 Hz, 1H), 5.17 (dd, J=8.5, 6.0 Hz, 1H), 4.65-4.60 (m, 1H), 4.48 (dd, J=9.3, 7.7 Hz, 1H), 4.36-4.31 (m, 1H), 3.72 (d, J=10.9 Hz, 1H), 3.66 (dd, J=11.1, 3.8 Hz, 1H), 3.16 (t, J=7.1 Hz, 2H), 3.07-2.82 (m, 11H), 2.72 (dd, J=14.1, 6.0 Hz, 1H), 2.61 (dd, J=14.1, 8.5 Hz, 1H), 2.58-2.50 (m, 1H), 2.36 (s, 3H), 2.11-2.00 (m, 2H), 1.88-1.82 (m, 1H), 1.45-1.38 (m, 2H), 1.33-1.05 (m, 7H), 0.95 (s, 9H). HRMS m/z [M+H]⁺ calcd for C₅₆H₆₇FN₉O₁₀S⁺ 1076.4710, found 1076.4715.

### Example 192

### Synthesis of LQ108-69

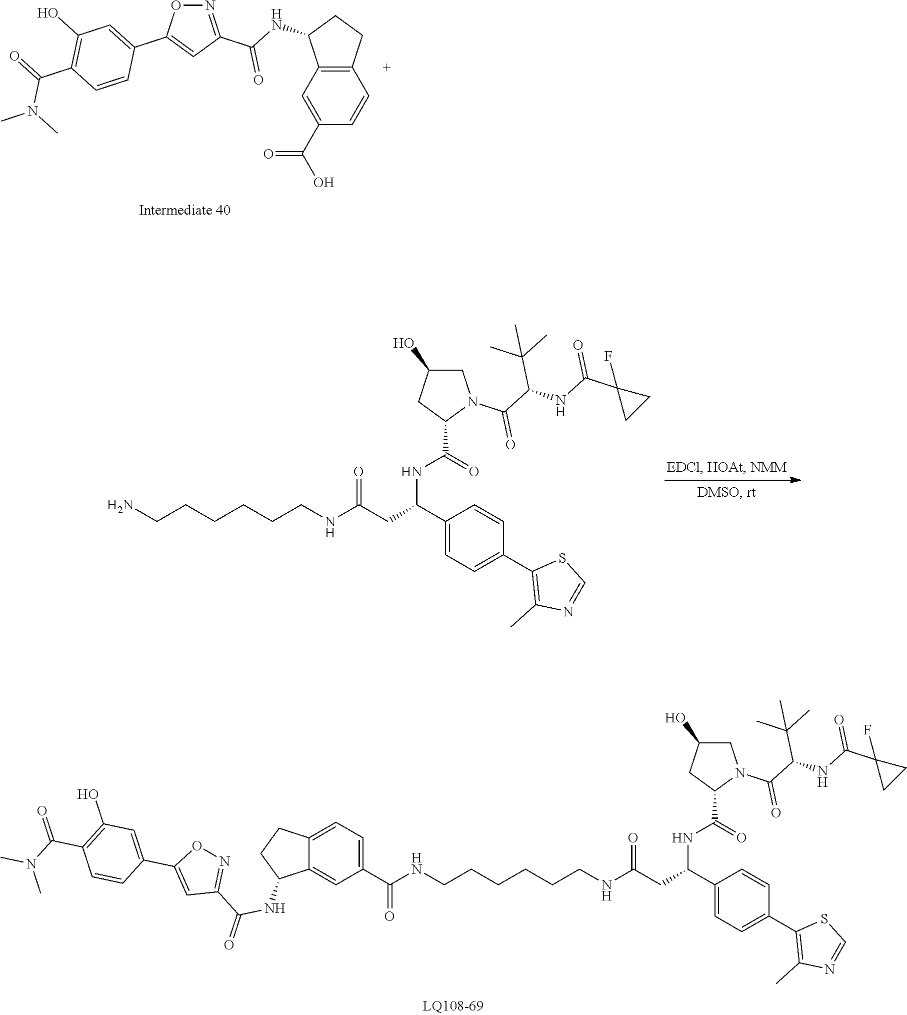 [SMILES: CN(C)C(=O)c1ccc(-c2cc(C(=O)N[C@@H]3CCc4ccc(C(=O)O)cc43)no2)cc1O ; Cc1ncsc1-c1ccc([C@H](CC(=O)NCCCCCCN)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1]

LQ108-69 was synthesized following the standard procedure for preparing LQ108-58 from intermediate 40 (5 mg, 0.01 mmol), (2S,4R)—N—((S)-3-((6-aminohexyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (7.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-69 was obtained as white solid (7.8 mg, 72%).

¹H NMR (600 MHz, Methanol-d₄) δ 9.01 (s, 1H), 7.77 (s, 1H), 7.72 (dd, J=7.9, 1.7 Hz, 1H), 7.51-7.39 (m, 5H), 7.37-7.31 (m, 3H), 7.15 (s, 1H), 5.69 (t, J=7.9 Hz, 1H), 5.29 (dd, J=8.4, 5.8 Hz, 1H), 4.75-4.71 (m, 1H), 4.61-4.55 (m, 1H), 4.46-4.41 (m, 1H), 3.85-3.80 (m, 1H), 3.76 (dd, J=11.1, 3.8 Hz, 1H), 3.26 (t, J=7.1 Hz, 2H), 3.17-2.92 (m, 11H), 2.83 (dd, J=14.1, 5.9 Hz, 1H), 2.73 (dd, J=14.1, 8.4 Hz, 1H), 2.68-2.61 (m, 1H), 2.47 (s, 3H), 2.21-2.08 (m, 2H), 1.98-1.92 (m, 1H), 1.54-1.46 (m, 2H), 1.40-1.24 (m, 7H), 1.21-1.13 (m, 2H), 1.05 (s, 9H). HRMS m/z [M+H]⁺ calcd for C₅₇H₆₉FN₉O₁₀S⁺ 1090.4867, found 1090.4860.

### Example 193

### Synthesis of LQ108-70

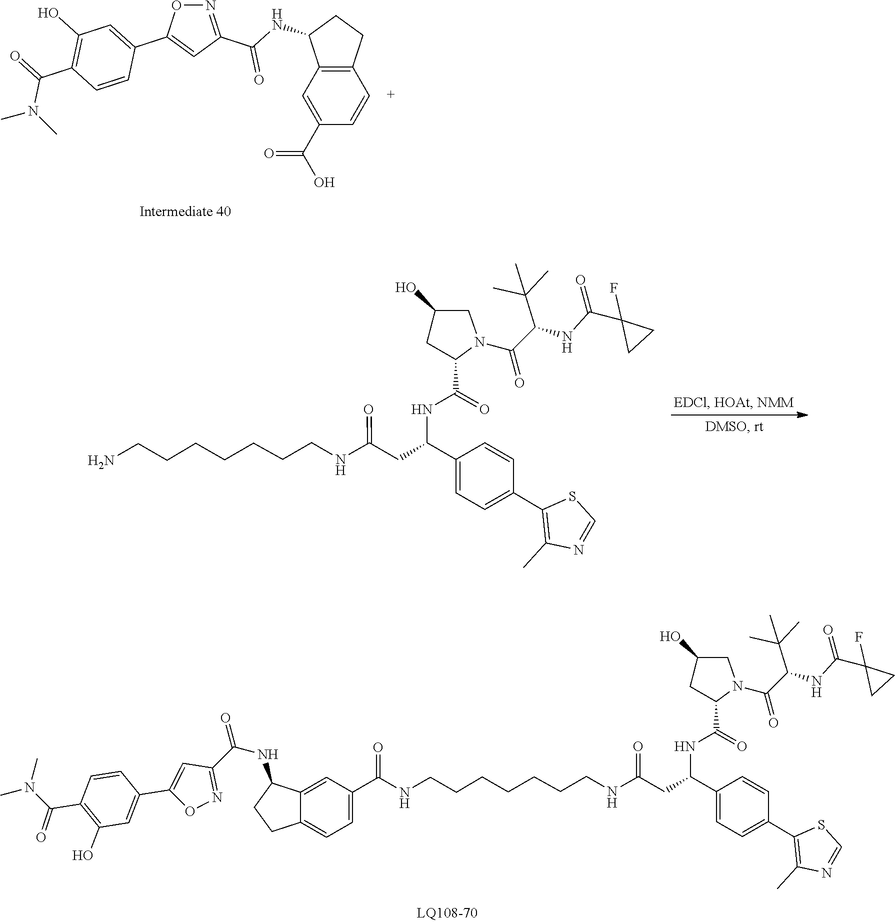

LQ108-70 was synthesized following the standard procedure for preparing LQ108-58 from intermediate 40 (5 mg, 0.01 mmol), (2S,4R)—N—((S)-3-((7-aminoheptyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (8.0 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-70 was obtained as white solid (7.4 mg, 67%).

¹H NMR (600 MHz, Methanol-d₄) δ 9.16 (s, 1H), 7.77 (s, 1H), 7.72 (dd, J=7.9, 1.7 Hz, 1H), 7.49-7.40 (m, 5H), 7.37-7.31 (m, 3H), 7.15 (s, 1H), 5.69 (t, J=7.9 Hz, 1H), 5.30 (dd, J=8.5, 5.9 Hz, 1H), 4.75-4.71 (m, 1H), 4.58 (dd, J=9.4, 7.7 Hz, 1H), 4.46-4.41 (m, 1H), 3.82 (d, J=11.1 Hz, 1H), 3.76 (dd, J=11.1, 3.8 Hz, 1H), 3.30-3.26 (m, 1H), 3.16-2.93 (m, 11H), 2.83 (dd, J=14.1, 5.9 Hz, 1H), 2.73 (dd, J=14.0, 8.5 Hz, 1H), 2.67-2.61 (m, 1H), 2.49 (s, 3H), 2.22-2.10 (m, 2H), 1.98-1.92 (m, 1H), 1.56-1.49 (m, 2H), 1.41-1.20 (m, 9H), 1.16-1.10 (m, 2H), 1.05 (s, 9H). HRMS m/z [M+H]⁺ calcd for C₅₈H₇₁FN₉O₁₀S⁺ 1104.5023, found 1104.5018.

### Example 194

### Synthesis of LQ108-71

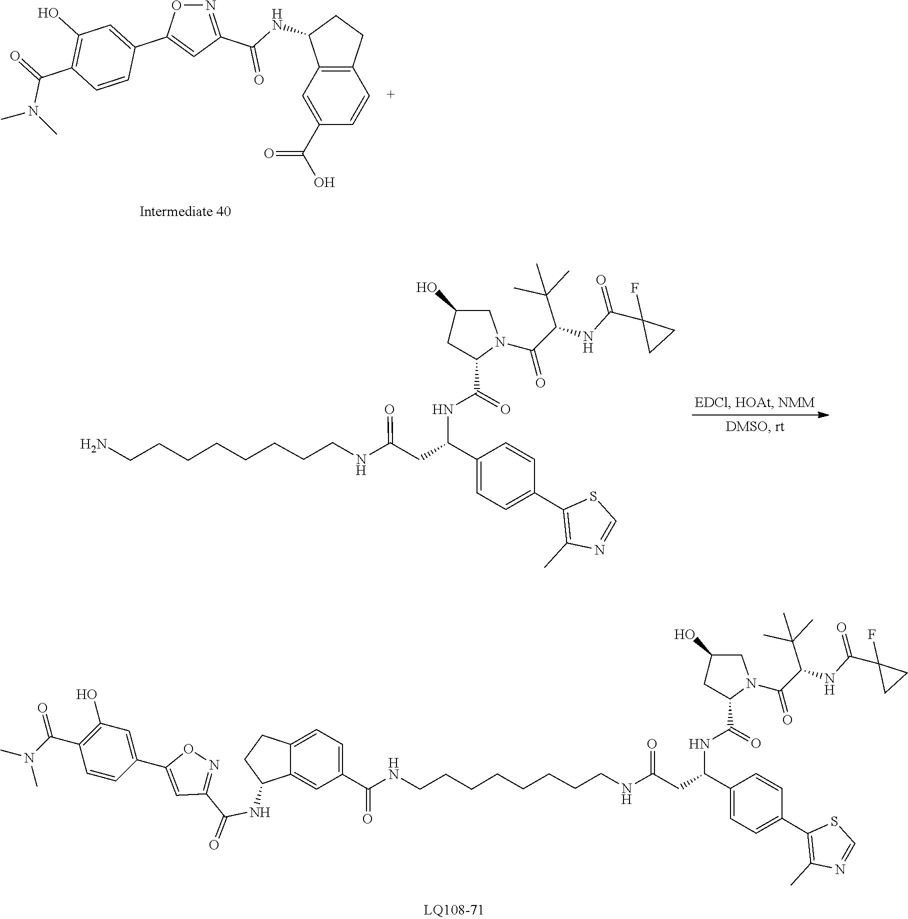

LQ108-71 was synthesized following the standard procedure for preparing LQ108-58 from intermediate 40 (5 mg, 0.01 mmol), (2S,4R)—N—((S)-3-((8-aminooctyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (8.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-71 was obtained as white solid (7 mg, 63%).

¹H NMR (600 MHz, Methanol-d₄) δ 9.08 (s, 1H), 7.78 (s, 1H), 7.73 (dd, J=7.9, 1.7 Hz, 1H), 7.50-7.39 (m, 5H), 7.38-7.31 (m, 3H), 7.14 (s, 1H), 5.69 (t, J=8.0 Hz, 1H), 5.30 (dd, J=8.5, 5.8 Hz, 1H), 4.76-4.71 (m, 1H), 4.58 (dd, J=9.3, 7.7 Hz, 1H), 4.46-4.42 (m, 1H), 3.82 (d, J=11.1 Hz, 1H), 3.76 (dd, J=11.1, 3.8 Hz, 1H), 3.30-3.26 (m, 1H), 3.17-2.91 (m, 11H), 2.83 (dd, J=14.0, 5.8 Hz, 1H), 2.72 (dd, J=14.0, 8.6 Hz, 1H), 2.68-2.61 (m, 1H), 2.48 (s, 3H), 2.22-2.10 (m, 2H), 1.98-1.92 (m, 1H), 1.51 (p, J=7.2 Hz, 2H), 1.41-1.15 (m, 11H), 1.14-1.08 (m, 2H), 1.05 (s, 9H). HRMS m/z [M+H]⁺ calcd for C₅₉H₇₃FN₉O₁₀S⁺ 1118.5180, found 1118.5183.

### Example 195

### Synthesis of LQ108-72

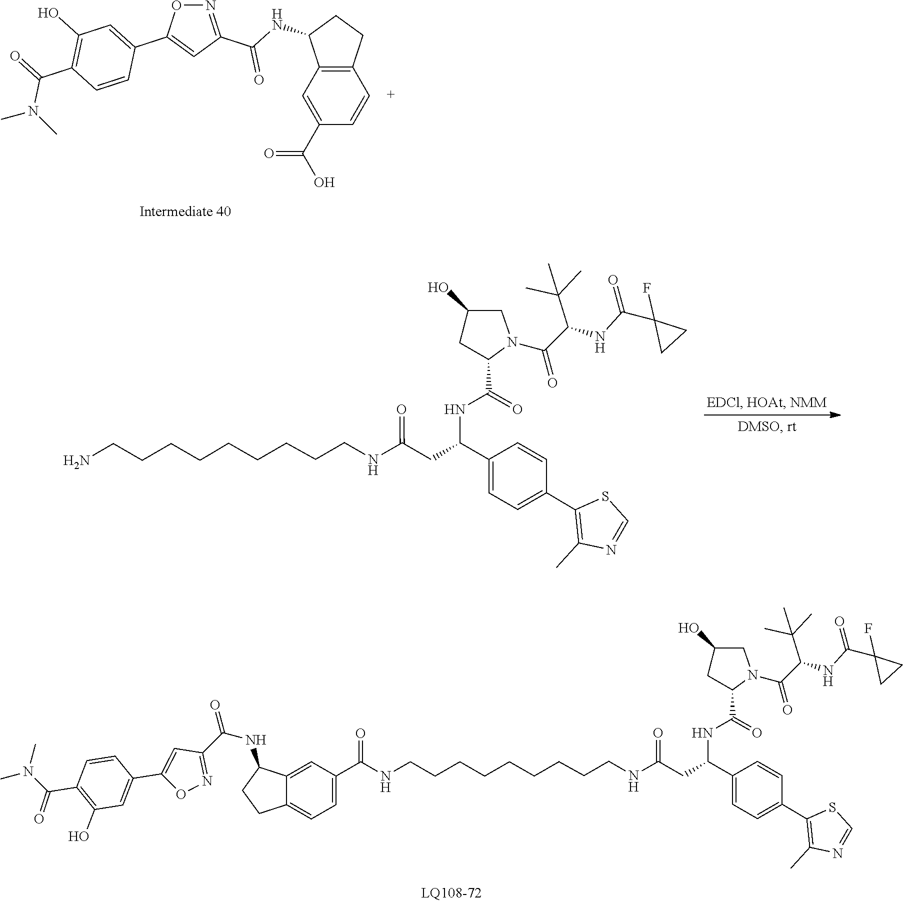

LQ108-72 was synthesized following the standard procedure for preparing LQ108-58 from intermediate 40 (5 mg, 0.01 mmol), (2S,4R)—N—((S)-3-((9-aminononyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (8.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-72 was obtained as white solid (7.7 mg, 68%).

¹H NMR (600 MHz, Methanol-d₄) δ 9.10 (s, 1H), 7.79 (s, 1H), 7.73 (dd, J=7.8, 1.7 Hz, 1H), 7.50-7.39 (m, 5H), 7.36 (d, J=7.9 Hz, 1H), 7.35-7.29 (m, 2H), 7.14 (s, 1H), 5.69 (t, J=7.9 Hz, 1H), 5.30 (dd, J=8.5, 5.8 Hz, 1H), 4.76-4.71 (m, 1H), 4.58 (dd, J=9.3, 7.7 Hz, 1H), 4.47-4.42 (m, 1H), 3.83 (d, J=11.0 Hz, 1H), 3.76 (dd, J=11.1, 3.8 Hz, 1H), 3.34-3.32 (m, 2H), 3.16-2.91 (m, 11H), 2.83 (dd, J=14.0, 5.8 Hz, 1H), 2.76-2.70 (m, 1H), 2.68-2.61 (m, 1H), 2.48 (s, 3H), 2.24-2.08 (m, 2H), 1.99-1.92 (m, 1H), 1.58-1.50 (m, 2H), 1.40-1.21 (m, 7H), 1.19-1.15 (m, 6H), 1.11-1.07 (m, 2H), 1.06 (s, 9H). HRMS m/z [M+H]⁺ calcd for C₆₀H₇₅FN₉O₁₀S⁺ 1132.5336, found 1132.5329.

### Example 196

### Synthesis of LQ108-73

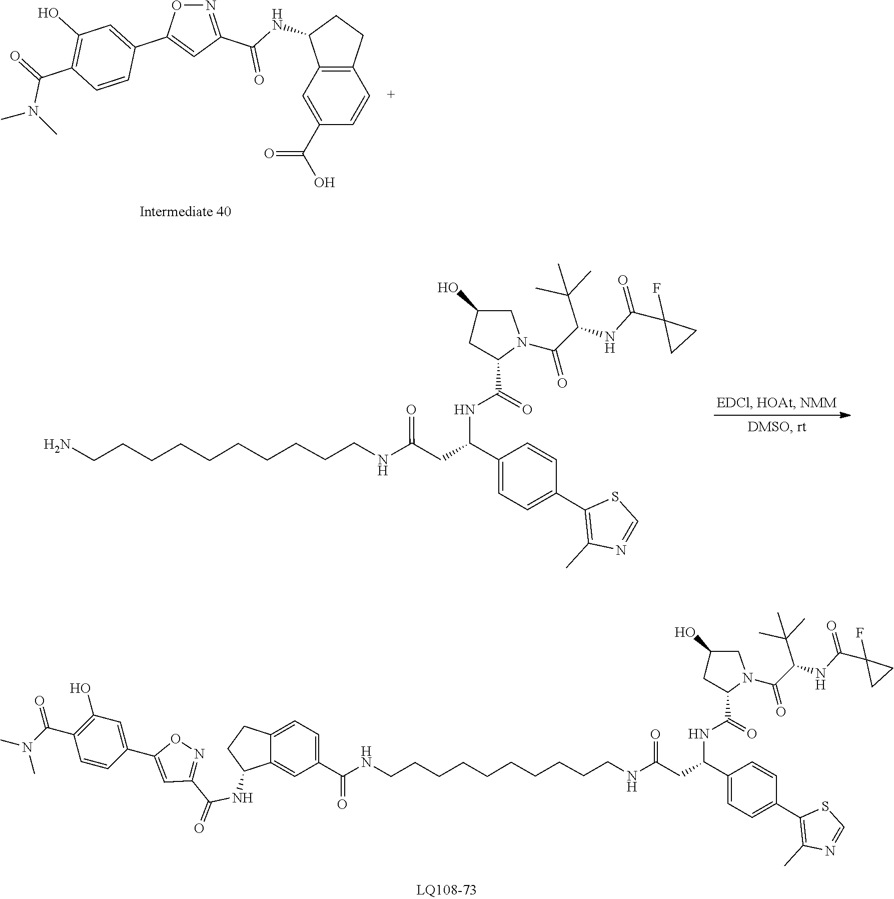

LQ108-73 was synthesized following the standard procedure for preparing LQ108-58 from intermediate 40 (5 mg, 0.01 mmol), (2S,4R)—N—((S)-3-((10-aminodecyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (8.4 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-73 was obtained as white solid (8.1 mg, 71%).

¹H NMR (600 MHz, Methanol-d₄) δ 9.03 (s, 1H), 7.78 (s, 1H), 7.73 (dd, J=8.0, 1.7 Hz, 1H), 7.51-7.39 (m, 5H), 7.38-7.31 (m, 3H), 7.14 (s, 1H), 5.69 (t, J=7.9 Hz, 1H), 5.30 (dd, J=8.5, 5.8 Hz, 1H), 4.76-4.72 (m, 1H), 4.58 (dd, J=9.2, 7.5 Hz, 1H), 4.46-4.42 (m, 1H), 3.83 (d, J=11.0 Hz, 1H), 3.76 (dd, J=11.1, 3.8 Hz, 1H), 3.36-3.32 (m, 2H), 3.16-2.92 (m, 11H), 2.83 (dd, J=14.0, 5.8 Hz, 1H), 2.77-2.70 (m, 1H), 2.69-2.61 (m, 1H), 2.48 (s, 3H), 2.23-2.16 (m, 1H), 2.16-2.07 (m, 1H), 1.98-1.92 (m, 1H), 1.56 (p, J=7.2 Hz, 2H), 1.38-1.13 (m, 15H), 1.10-1.07 (m, 2H), 1.06 (s, 9H). HRMS m/z [M+H]⁺ calcd for C₆₁H₇₇FN₉O₁₀S⁺ 1146.5493, found 1146.5490.

### Example 197

### Synthesis of LQ108-74

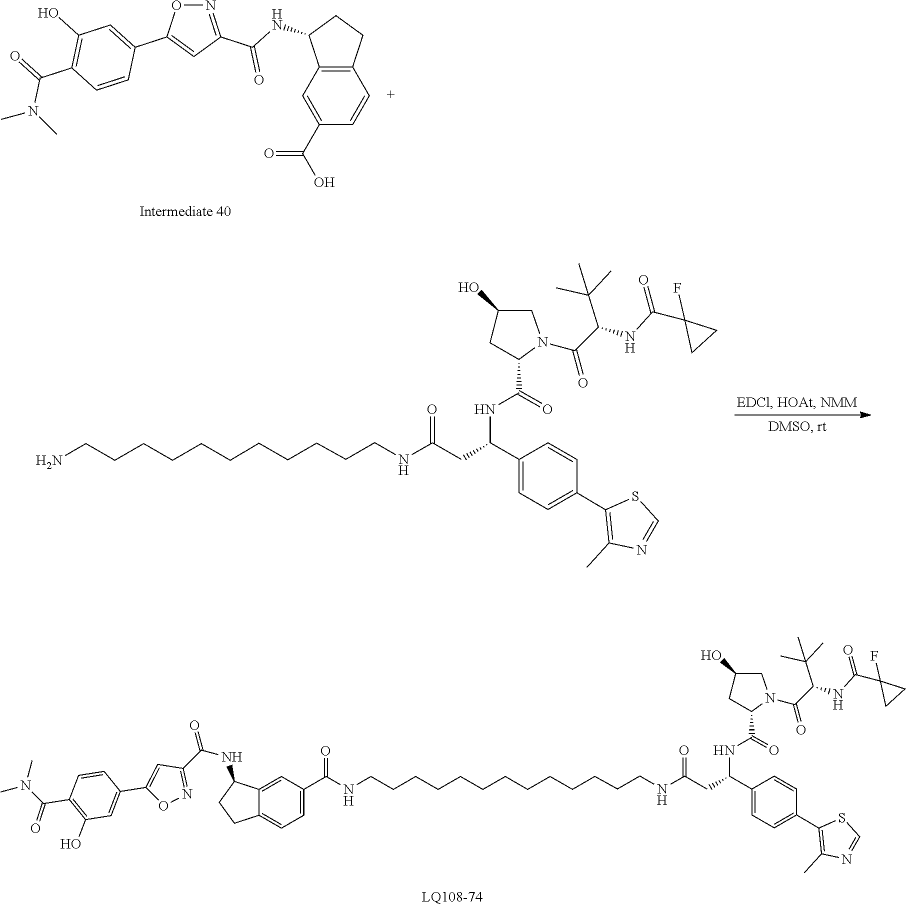

LQ108-74 was synthesized following the standard procedure for preparing LQ108-58 from intermediate 40 (5 mg, 0.01 mmol), (2S,4R)—N—((S)-3-((11-aminoundecyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (8.5 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-74 was obtained as white solid (8.9 mg, 77%).

¹H NMR (600 MHz, Methanol-d₄) δ 9.11 (s, 1H), 7.78 (s, 1H), 7.73 (dd, J=8.0, 1.7 Hz, 1H), 7.51-7.40 (m, 5H), 7.38-7.31 (m, 3H), 7.14 (s, 1H), 5.69 (t, J=7.9 Hz, 1H), 5.31 (dd, J=8.5, 5.8 Hz, 1H), 4.76-4.71 (m, 1H), 4.58 (dd, J=9.2, 7.6 Hz, 1H), 4.46-4.42 (m, 1H), 3.83 (d, J=11.1 Hz, 1H), 3.76 (dd, J=11.1, 3.8 Hz, 1H), 3.36-3.32 (m, 2H), 3.16-2.94 (m, 11H), 2.84 (dd, J=14.0, 5.8 Hz, 1H), 2.73 (dd, J=14.0, 8.6 Hz, 1H), 2.68-2.61 (m, 1H), 2.49 (s, 3H), 2.23-2.16 (m, 1H), 2.15-2.07 (m, 1H), 1.99-1.92 (m, 1H), 1.58 (p, J=7.2 Hz, 2H), 1.41-1.25 (m, 9H), 1.23-1.12 (m, 8H), 1.11-1.07 (m, 2H), 1.06 (s, 9H). HRMS m/z [M+H]⁺ calcd for C₆₂H₇₉FN₉O₁₀S⁺ 1160.5649, found 1160.5652.

### Example 198

### Synthesis of LQ108-75

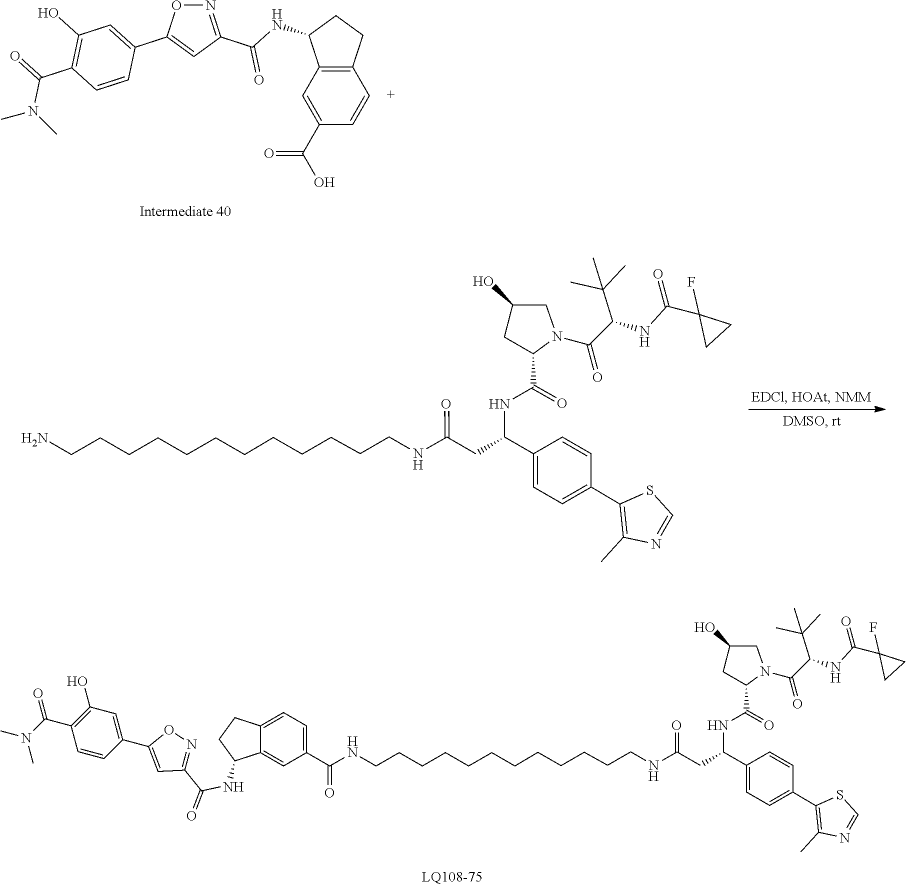

LQ108-75 was synthesized following the standard procedure for preparing LQ108-58 from intermediate 40 (5 mg, 0.01 mmol), (2S,4R)—N—((S)-3-((12-aminododecyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (8.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ108-75 was obtained as white solid (8.6 mg, 73%).

¹H NMR (600 MHz, Methanol-d₄) δ 9.04 (s, 1H), 7.78 (s, 1H), 7.73 (dd, J=7.9, 1.7 Hz, 1H), 7.52-7.40 (m, 5H), 7.38-7.31 (m, 3H), 7.14 (s, 1H), 5.69 (t, J=7.9 Hz, 1H), 5.31 (dd, J=8.5, 5.8 Hz, 1H), 4.74 (dd, J=9.3, 1.3 Hz, 1H), 4.58 (dd, J=9.2, 7.5 Hz, 1H), 4.47-4.42 (m, 1H), 3.85-3.80 (m, 1H), 3.76 (dd, J=11.1, 3.8 Hz, 1H), 3.37-3.32 (m, 2H), 3.16-2.94 (m, 11H), 2.84 (dd, J=14.0, 5.8 Hz, 1H), 2.74 (dd, J=14.0, 8.5 Hz, 1H), 2.68-2.60 (m, 1H), 2.48 (s, 3H), 2.23-2.16 (m, 1H), 2.15-2.07 (m, 1H), 1.99-1.92 (m, 1H), 1.59 (p, J=7.2 Hz, 2H), 1.42-1.13 (m, 19H), 1.12-1.07 (m, 2H), 1.06 (s, 9H). HRMS m/z [M+H]⁺ calcd for C₆₃H₈₁FN₉O₁₀S⁺ 1174.5806, found 1174.5795.

### Example 199

### Synthesis of LQ126-46

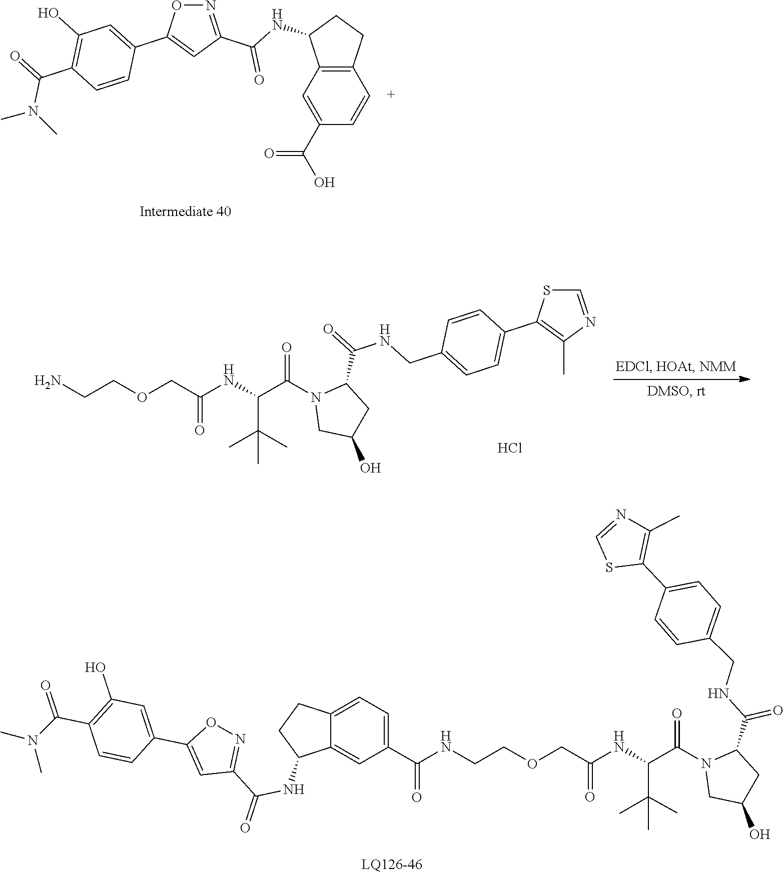 [SMILES: CN(C)C(=O)c1ccc(-c2cc(C(=O)N[C@@H]3CCc4ccc(C(=O)O)cc43)no2)cc1O ; Cc1ncsc1-c1ccc(CNC(=O)[C@@H]2C[C@@H](O)CN2C(=O)[C@@H](NC(=O)COCCN)C(C)(C)C)cc1 ; Cl ; Cc1ncsc1-c1ccc(CNC(=O)[C@@H]2C[C@@H](O)CN2C(=O)[C@@H](NC(=O)COCCNC(=O)c2ccc3c(c2)[C@H](NC(=O)c2cc(-c4ccc(C(=O)N(C)C)c(O)c4)on2)CC3)C(C)(C)C)cc1]

LQ126-46 was synthesized following the standard procedure for preparing LQ108-58 from intermediate 40 (5 mg, 0.01 mmol), (2S,4R)-1-((S)-2-(2-(2-aminoethoxy)acetamido)-3,3-dimethylbutanoyl)-4-hydroxy-N-(4-(4-methylthiazol-5-yl)benzyl)pyrrolidine-2-carboxamide (5.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-46 was obtained as white solid (6.3 mg, 66%). ¹H NMR (600 MHz, Methanol-d₄) δ 9.14 (s, 1H), 7.81 (s, 1H), 7.78-7.75 (m, 1H), 7.47 (d, J=8.1 Hz, 2H), 7.43-7.37 (m, 3H), 7.35-7.28 (m, 3H), 7.14 (s, 1H), 5.64 (t, J=7.8 Hz, 1H), 4.70-4.66 (m, 1H), 4.62-4.56 (m, 2H), 4.51-4.48 (m, 1H), 4.34 (d, J=15.7 Hz, 1H), 4.09-3.97 (m, 2H), 3.86 (d, J=11.0 Hz, 1H), 3.79 (dd, J=11.0, 3.8 Hz, 1H), 3.76-3.55 (m, 3H), 3.18-2.91 (m, 8H), 2.91-2.82 (m, 1H), 2.60-2.54 (m, 1H), 2.47 (s, 3H), 2.27-2.20 (m, 1H), 2.13-2.03 (m, 2H), 0.98 (s, 9H). HRMS m/z [M+H]⁺ calcd for C₄₉H₅₇N₈O₁₀S⁺ 949.3913, found 949.3911.

### Example 200

### Synthesis of LQ126-47

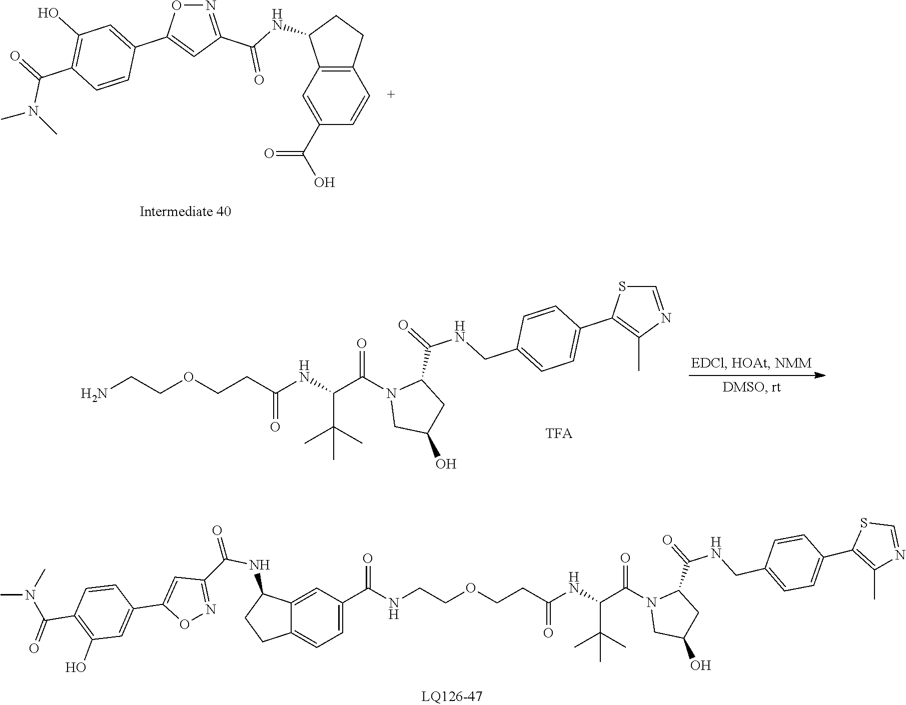 [SMILES: CN(C)C(=O)c1ccc(-c2cc(C(=O)N[C@@H]3CCc4ccc(C(=O)O)cc43)no2)cc1O ; Cc1ncsc1-c1ccc(CNC(=O)[C@@H]2C[C@@H](O)CN2C(=O)[C@@H](NC(=O)CCOCCN)C(C)(C)C)cc1 ; Cc1ncsc1-c1ccc(CNC(=O)[C@@H]2C[C@@H](O)CN2C(=O)[C@@H](NC(=O)CCOCCNC(=O)c2ccc3c(c2)[C@H](NC(=O)c2cc(-c4ccc(C(=O)N(C)C)c(O)c4)on2)CC3)C(C)(C)C)cc1 ; "TFA"]

LQ126-47 was synthesized following the standard procedure for preparing LQ108-58 from intermediate 40 (5 mg, 0.01 mmol), (2S,4R)-1-((S)-2-(3-(2-aminoethoxy)propanamido)-3,3-dimethylbutanoyl)-4-hydroxy-N-(4-(4-methylthiazol-5-yl)benzyl)pyrrolidine-2-carboxamide (6.4 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-47 was obtained as white solid (6.7 mg, 70%). ¹H NMR (600 MHz, Methanol-d₄) δ 8.97 (s, 1H), 7.80-7.78 (m, 1H), 7.76-7.73 (m, 1H), 7.46-7.31 (m, 8H), 7.15 (s, 1H), 5.67 (t, J=7.9 Hz, 1H), 4.64-4.60 (m, 1H), 4.58-4.46 (m, 3H), 4.33 (d, J=15.6 Hz, 1H), 3.86 (d, J=10.9 Hz, 1H), 3.80-3.69 (m, 3H), 3.65-3.51 (m, 4H), 3.16-2.90 (m, 8H), 2.67-2.59 (m, 1H), 2.57-2.44 (m, 5H), 2.24-2.17 (m, 1H), 2.14-2.03 (m, 2H), 0.99 (s, 9H). HRMS m/z [M+H]⁺ calcd for C₅₀H₅₉N₈O₁₀S⁺ 963.4069, found 963.4070.

### Example 201

### Synthesis of LQ126-49

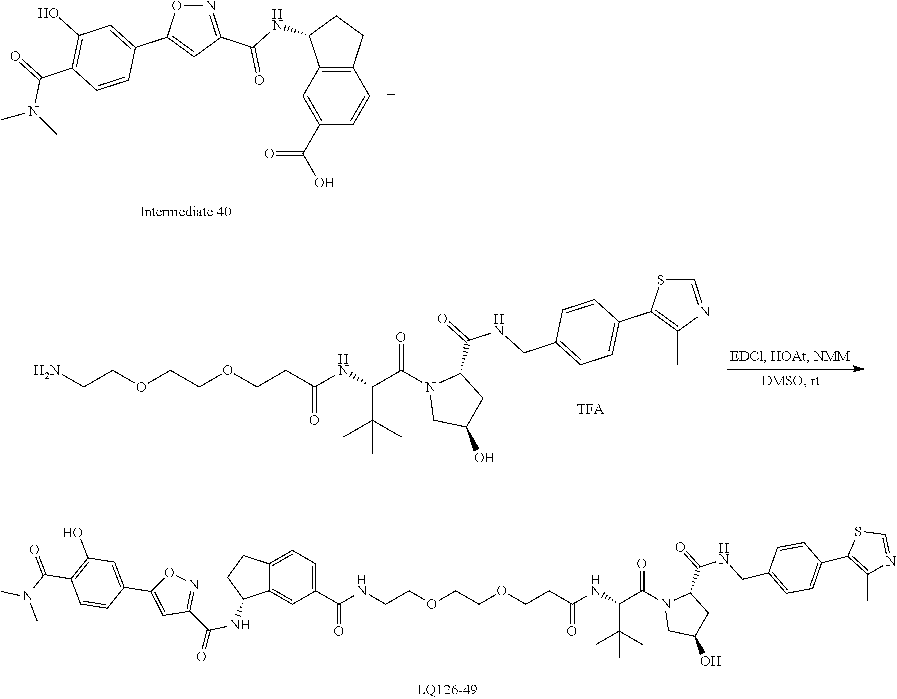 [SMILES: CN(C)C(=O)c1ccc(-c2cc(C(=O)N[C@@H]3CCc4ccc(C(=O)O)cc43)no2)cc1O ; Cc1ncsc1-c1ccc(CNC(=O)[C@@H]2C[C@@H](O)CN2C(=O)[C@@H](NC(=O)CCOCCOCCN)C(C)(C)C)cc1 ; Cc1ncsc1-c1ccc(CNC(=O)[C@@H]2C[C@@H](O)CN2C(=O)[C@@H](NC(=O)CCOCCOCCNC(=O)c2ccc3c(c2)[C@H](NC(=O)c2cc(-c4ccc(C(=O)N(C)C)c(O)c4)on2)CC3)C(C)(C)C)cc1 ; "TFA"]

LQ126-49 was synthesized following the standard procedure for preparing LQ108-58 from intermediate 40 (5 mg, 0.01 mmol), (2S,4R)-1-((S)-2-(3-(2-(2-aminoethoxy)ethoxy)propanamido)-3,3-dimethylbutanoyl)-4-hydroxy-N-(4-(4-methylthiazol-5-yl)benzyl)pyrrolidine-2-carboxamide (6.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-49 was obtained as white solid (6.5 mg, 65%). ¹H NMR (600 MHz, Methanol-d₄) δ 9.01 (s, 1H), 7.78 (s, 1H), 7.76-7.71 (m, 1H), 7.47-7.31 (m, 8H), 7.18 (s, 1H), 5.68 (t, J=7.9 Hz, 1H), 4.63 (s, 1H), 4.58-4.46 (m, 3H), 4.33 (d, J=15.5 Hz, 1H), 3.86 (d, J=10.8 Hz, 1H), 3.77 (dd, J=11.0, 3.9 Hz, 1H), 3.72-3.66 (m, 2H), 3.64-3.57 (m, 6H), 3.55-3.49 (m, 2H), 3.16-2.90 (m, 8H), 2.67-2.59 (m, 1H), 2.53-2.40 (m, 5H), 2.21 (dd, J=13.1, 7.8 Hz, 1H), 2.16-2.03 (m, 2H), 1.01 (s, 9H). HRMS m/z [M+H]⁺ calcd for C₅₂H₆₃N₈O₁₁S⁺ 1007.4332, found 1007.4335.

### Example 202

### Synthesis of LQ126-50

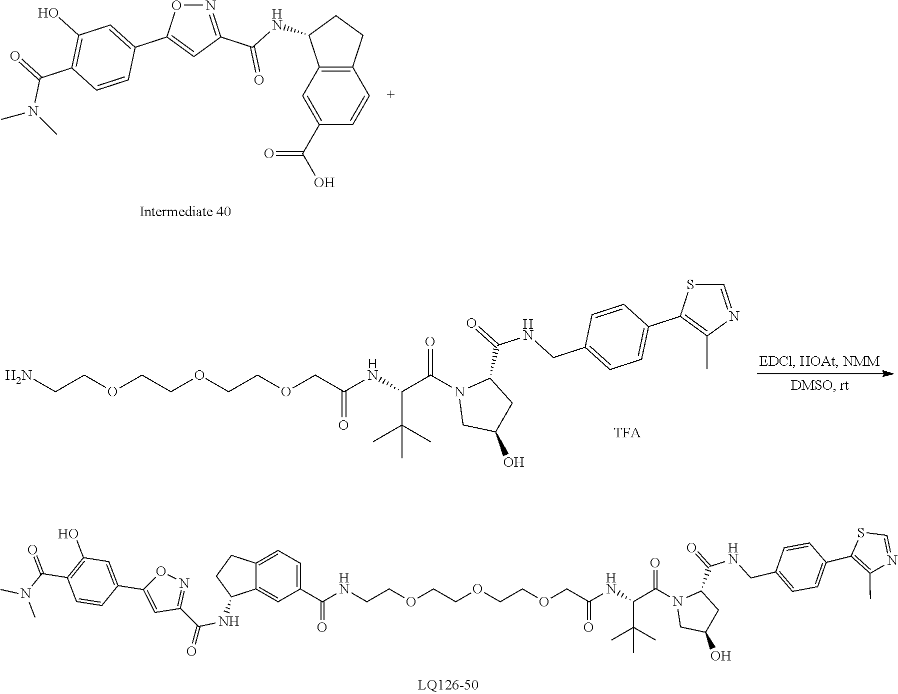 [SMILES: CN(C)C(=O)c1ccc(-c2cc(C(=O)N[C@@H]3CCc4ccc(C(=O)O)cc43)no2)cc1O ; Cc1ncsc1-c1ccc(CNC(=O)[C@@H]2C[C@@H](O)CN2C(=O)[C@@H](NC(=O)COCCOCCOCCN)C(C)(C)C)cc1 ; Cc1ncsc1-c1ccc(CNC(=O)[C@@H]2C[C@@H](O)CN2C(=O)[C@@H](NC(=O)COCCOCCOCCNC(=O)c2ccc3c(c2)[C@H](NC(=O)c2cc(-c4ccc(C(=O)N(C)C)c(O)c4)on2)CC3)C(C)(C)C)cc1 ; "TFA"]

LQ126-50 was synthesized following the standard procedure for preparing LQ108-58 from intermediate 40 (5 mg, 0.01 mmol), (2S,4R)-1-((S)-14-amino-2-(tert-butyl)-4-oxo-6,9,12-trioxa-3-azatetradecanoyl)-4-hydroxy-N-(4-(4-methylthiazol-5-yl)benzyl)pyrrolidine-2-carboxamide (7.2 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-50 was obtained as white solid (6.3 mg, 61%). ¹H NMR (600 MHz, Methanol-d₄) δ 9.14 (s, 1H), 7.77 (s, 1H), 7.74-7.70 (m, 1H), 7.48-7.39 (m, 5H), 7.37-7.32 (m, 3H), 7.16 (s, 1H), 5.67 (t, J=8.0 Hz, 1H), 4.68 (s, 1H), 4.61-4.47 (m, 3H), 4.34 (d, J=15.5 Hz, 1H), 4.00-3.95 (m, 1H), 3.91-3.83 (m, 2H), 3.78 (dd, J=11.0, 3.8 Hz, 1H), 3.68-3.57 (m, 10H), 3.55-3.50 (m, 2H), 3.17-2.92 (m, 8H), 2.67-2.59 (m, 1H), 2.48 (s, 3H), 2.26-2.19 (m, 1H), 2.15-2.04 (m, 2H), 1.02 (s, 9H). HRMS m/z [M+H]⁺ calcd for C₅₃H₆₅N₈O₁₂S⁺ 1037.4437, found 1037.4432.

### Example 203

### Synthesis of LQ126-51

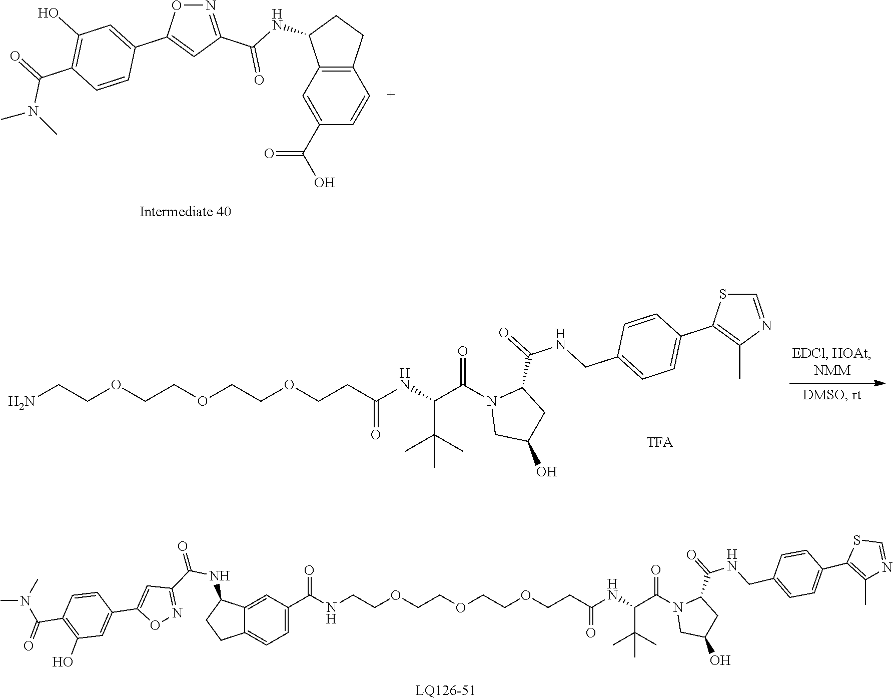 [SMILES: CN(C)C(=O)c1ccc(-c2cc(C(=O)N[C@@H]3CCc4ccc(C(=O)O)cc43)no2)cc1O ; Cc1ncsc1-c1ccc(CNC(=O)[C@@H]2C[C@@H](O)CN2C(=O)[C@@H](NC(=O)CCOCCOCCOCCN)C(C)(C)C)cc1 ; Cc1ncsc1-c1ccc(CNC(=O)[C@@H]2C[C@@H](O)CN2C(=O)[C@@H](NC(=O)CCOCCOCCOCCNC(=O)c2ccc3c(c2)[C@H](NC(=O)c2cc(-c4ccc(C(=O)N(C)C)c(O)c4)on2)CC3)C(C)(C)C)cc1 ; "TFA"]

LQ126-51 was synthesized following the standard procedure for preparing LQ108-58 from intermediate 40 (5 mg, 0.01 mmol), (2S,4R)-1-((S)-1-amino-14-(tert-butyl)-12-oxo-3,6,9-trioxa-13-azapentadecan-15-oyl)-4-hydroxy-N-(4-(4-methylthiazol-5-yl)benzyl)pyrrolidine-2-carboxamide (7.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-51 was obtained as white solid (5.8 mg, 55%). ¹H NMR (600 MHz, Methanol-d₄) δ 9.05 (s, 1H), 7.78 (s, 1H), 7.74 (dd, J=7.9, 1.7 Hz, 1H), 7.48-7.45 (m, 2H), 7.44-7.39 (m, 3H), 7.37-7.32 (m, 3H), 7.17 (s, 1H), 5.69 (t, J=7.9 Hz, 1H), 4.63 (s, 1H), 4.59-4.51 (m, 2H), 4.50-4.47 (m, 1H), 4.34 (d, J=15.5 Hz, 1H), 3.87 (d, J=11.0 Hz, 1H), 3.78 (dd, J=10.9, 3.9 Hz, 1H), 3.70-3.50 (m, 14H), 3.16-2.91 (m, 8H), 2.68-2.59 (m, 1H), 2.53-2.45 (m, 4H), 2.45-2.36 (m, 1H), 2.24-2.18 (m, 1H), 2.14-2.03 (m, 2H), 1.02 (s, 9H). HRMS m/z [M+H]⁺ calcd for C₅₄H₆₇N₈O₁₂S⁺ 1051.4594, found 1051.4594.

### Example 204

### Synthesis of LQ126-52

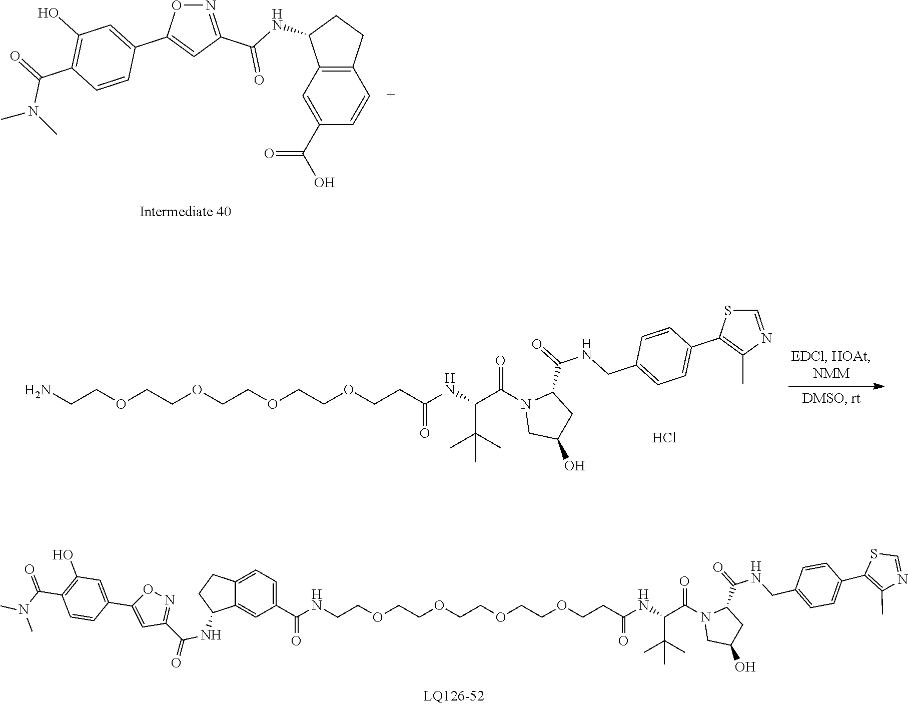 [SMILES: CN(C)C(=O)c1ccc(-c2cc(C(=O)N[C@@H]3CCc4ccc(C(=O)O)cc43)no2)cc1O ; Cc1ncsc1-c1ccc(CNC(=O)[C@@H]2C[C@@H](O)CN2C(=O)[C@@H](NC(=O)CCOCCOCCOCCOCCN)C(C)(C)C)cc1 ; Cl ; Cc1ncsc1-c1ccc(CNC(=O)[C@@H]2C[C@@H](O)CN2C(=O)[C@@H](NC(=O)CCOCCOCCOCCOCCNC(=O)c2ccc3c(c2)[C@H](NC(=O)c2cc(-c4ccc(C(=O)N(C)C)c(O)c4)on2)CC3)C(C)(C)C)cc1]

LQ126-52 was synthesized following the standard procedure for preparing LQ108-58 from intermediate 40 (5 mg, 0.01 mmol), (2S,4R)-1-((S)-1-amino-17-(tert-butyl)-15-oxo-3,6,9,12-tetraoxa-16-azaoctadecan-18-oyl)-4-hydroxy-N-(4-(4-methylthiazol-5-yl)benzyl)pyrrolidine-2-carboxamide (7.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-52 was obtained as white solid (7.3 mg, 67%). ¹H NMR (600 MHz, Methanol-d₄) δ 9.16 (s, 1H), 7.79 (s, 1H), 7.75 (dd, J=7.9, 1.7 Hz, 1H), 7.50-7.46 (m, 2H), 7.44-7.40 (m, 3H), 7.38-7.32 (m, 3H), 7.16 (s, 1H), 5.69 (t, J=7.9 Hz, 1H), 4.64 (s, 1H), 4.59-4.52 (m, 2H), 4.50-4.47 (m, 1H), 4.35 (d, J=15.5 Hz, 1H), 3.91-3.85 (m, 1H), 3.79 (dd, J=11.0, 3.9 Hz, 1H), 3.73-3.51 (m, 18H), 3.17-2.92 (m, 8H), 2.68-2.60 (m, 1H), 2.57-2.50 (m, 1H), 2.49 (s, 3H), 2.47-2.42 (m, 1H), 2.25-2.19 (m, 1H), 2.15-2.04 (m, 2H), 1.02 (s, 9H). HRMS m/z [M+H]⁺ calcd for C₅₆H₇₁N₈O₁₃S⁺ 1095.4856, found 1095.4853.

### Example 205

### Synthesis of LQ126-53

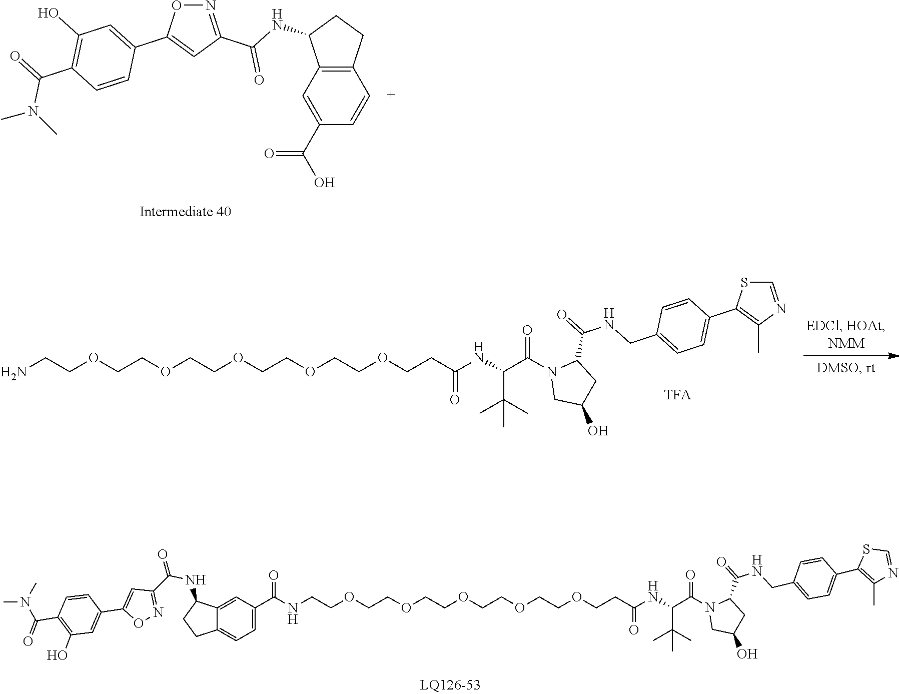

LQ126-53 was synthesized following the standard procedure for preparing LQ108-58 from intermediate 40 (5 mg, 0.01 mmol), (2S,4R)-1-((S)-1-amino-20-(tert-butyl)-18-oxo-3,6,9,12,15-pentaoxa-19-azahenicosan-21-oyl)-4-hydroxy-N-(4-(4-methylthiazol-5-yl)benzyl)pyrrolidine-2-carboxamide (7.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-53 was obtained as white solid (6.4 mg, 62%). ¹H NMR (600 MHz, Methanol-d₄) δ 9.21 (s, 1H), 7.79 (s, 1H), 7.76-7.73 (m, 1H), 7.51-7.47 (m, 2H), 7.45-7.41 (m, 3H), 7.38-7.32 (m, 3H), 7.16 (s, 1H), 5.69 (t, J=7.9 Hz, 1H), 4.64 (s, 1H), 4.59-4.52 (m, 2H), 4.51-4.47 (m, 1H), 4.35 (d, J=15.6 Hz, 1H), 3.90-3.86 (m, 1H), 3.79 (dd, J=11.0, 3.9 Hz, 1H), 3.74-3.67 (m, 2H), 3.65-3.51 (m, 20H), 3.16-2.92 (m, 8H), 2.67-2.60 (m, 1H), 2.59-2.52 (m, 1H), 2.50 (s, 3H), 2.48-2.42 (m, 1H), 2.24-2.18 (m, 1H), 2.15-2.03 (m, 2H), 1.03 (s, 9H). HRMS m/z [M+H]+ calcd for C₅₈H₇₅N₈O₁₄S⁺ 1139.5118, found 1139.5113.

### Example 206

### Synthesis of LQ126-54

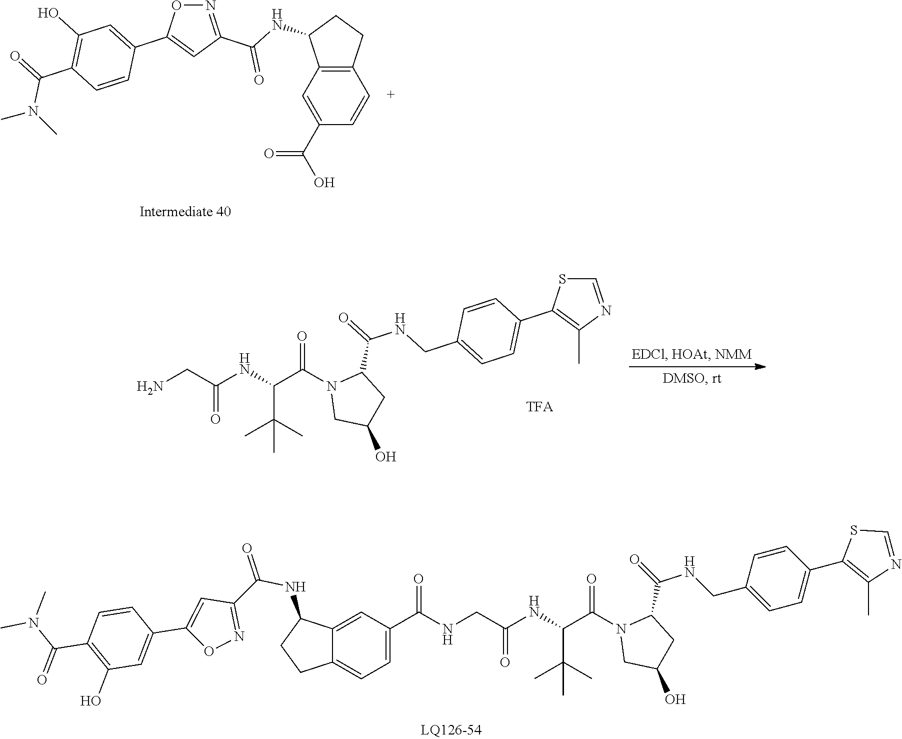

LQ126-54 was synthesized following the standard procedure for preparing LQ108-58 from intermediate 40 (5 mg, 0.01 mmol), (2S,4R)-1-((S)-2-(2-aminoacetamido)-3,3-dimethylbutanoyl)-4-hydroxy-N-(4-(4-methylthiazol-5-yl)benzyl)pyrrolidine-2-carboxamide (5.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-54 was obtained as white solid (6.7 mg, 74%). ¹H NMR (600 MHz, Methanol-d₄) δ 9.09 (s, 1H), 7.86-7.77 (m, 2H), 7.49-7.45 (m, 2H), 7.43-7.31 (m, 6H), 7.14 (s, 1H), 5.68 (t, J=7.9 Hz, 1H), 4.63 (s, 1H), 4.58-4.52 (m, 2H), 4.50-4.45 (m, 1H), 4.34 (d, J=15.6 Hz, 1H), 4.05 (d, J=2.3 Hz, 2H), 3.87 (d, J=11.0 Hz, 1H), 3.78 (dd, J=11.0, 3.8 Hz, 1H), 3.17-2.92 (m, 8H), 2.68-2.60 (m, 1H), 2.47 (s, 3H), 2.23-2.17 (m, 1H), 2.15-2.03 (m, 2H), 1.00 (s, 9H). HRMS m/z [M+H]⁺ calcd for C₄₇H₅₃N₈O₉S⁺ 905.3651, found 905.3638.

### Example 207

### Synthesis of LQ126-55

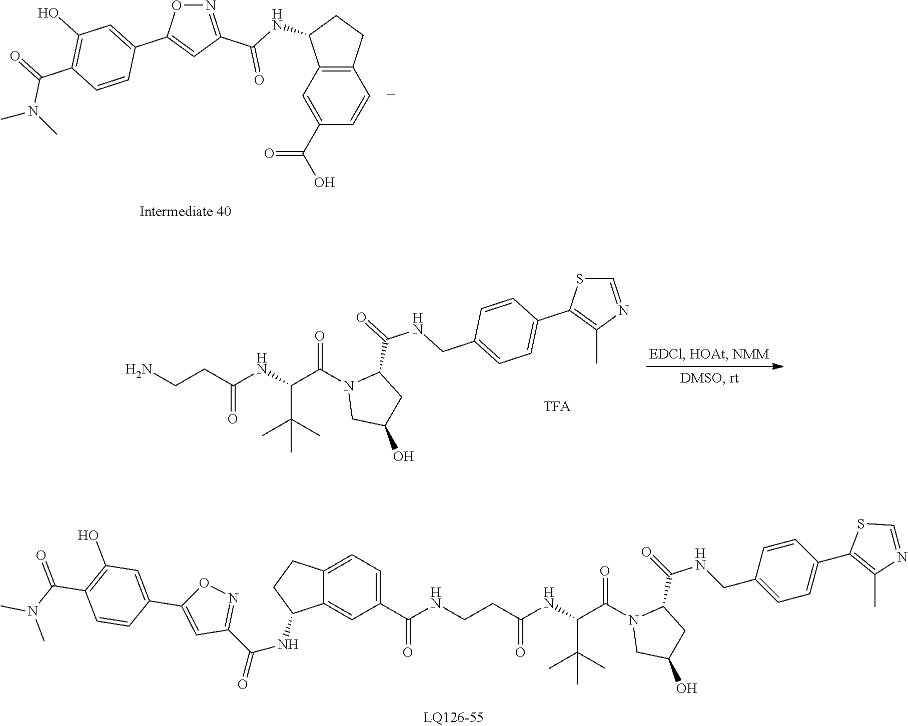 [SMILES: CN(C)C(=O)c1ccc(-c2cc(C(=O)N[C@@H]3CCc4ccc(C(=O)O)cc43)no2)cc1O ; Cc1ncsc1-c1ccc(CNC(=O)[C@@H]2C[C@@H](O)CN2C(=O)[C@@H](NC(=O)CCN)C(C)(C)C)cc1 ; Cc1ncsc1-c1ccc(CNC(=O)[C@@H]2C[C@@H](O)CN2C(=O)[C@@H](NC(=O)CCNC(=O)c2ccc3c(c2)[C@H](NC(=O)c2cc(-c4ccc(C(=O)N(C)C)c(O)c4)on2)CC3)C(C)(C)C)cc1]

LQ126-55 was synthesized following the standard procedure for preparing LQ108-58 from intermediate 40 (5 mg, 0.01 mmol), (2S,4R)-1-((S)-2-(3-aminopropanamido)-3,3-dimethylbutanoyl)-4-hydroxy-N-(4-(4-methylthiazol-5-yl)benzyl)pyrrolidine-2-carboxamide (6 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-55 was obtained as white solid (6.2 mg, 68%). ¹H NMR (600 MHz, Methanol-d₄) δ 9.07 (s, 1H), 7.98-7.92 (m, 1H), 7.76-7.72 (m, 1H), 7.49-7.31 (m, 8H), 7.16-7.15 (m, 1H), 5.66 (t, J=7.6 Hz, 1H), 4.59 (s, 1H), 4.57-4.46 (m, 3H), 4.34 (d, J=15.5 Hz, 1H), 3.90 (d, J=10.9 Hz, 1H), 3.76 (dd, J=11.0, 3.9 Hz, 1H), 3.67-3.53 (m, 2H), 3.18-2.88 (m, 8H), 2.68-2.52 (m, 3H), 2.48 (s, 3H), 2.20 (dd, J=13.3, 7.7 Hz, 1H), 2.17-2.03 (m, 2H), 0.97 (s, 9H). HRMS m/z [M+H]⁺ calcd for C₄₈H₅₅N₈O₉S⁺ 919.3807, found 919.3798.

### Example 208

### Synthesis of LQ126-56

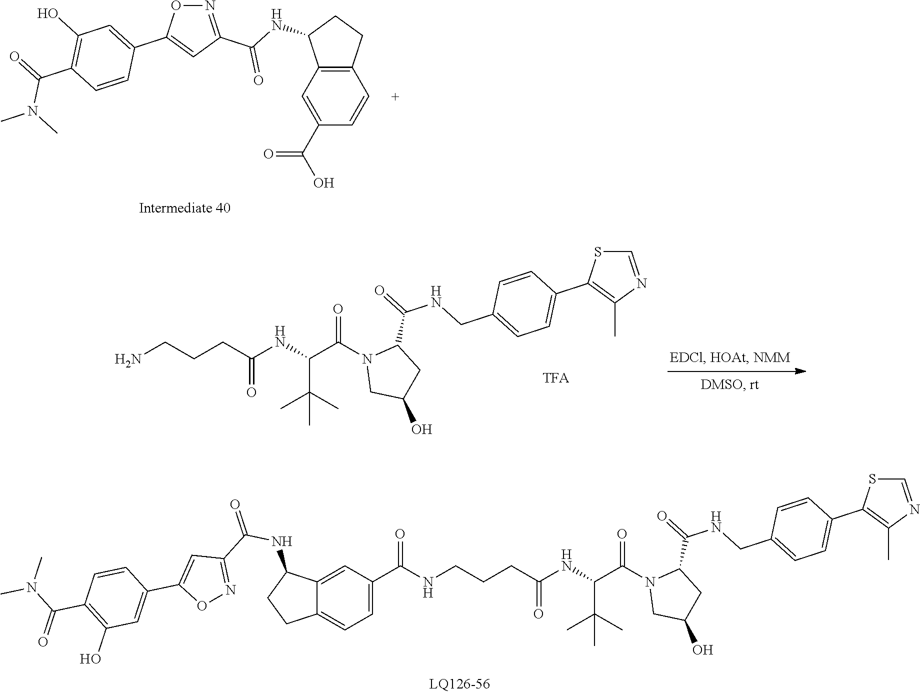

LQ126-56 was synthesized following the standard procedure for preparing LQ108-58 from intermediate 40 (5 mg, 0.01 mmol), (2S,4R)-1-((S)-2-(4-aminobutanamido)-3,3-dimethylbutanoyl)-4-hydroxy-N-(4-(4-methylthiazol-5-yl)benzyl)pyrrolidine-2-carboxamide (6.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-56 was obtained as white solid (5.5 mg, 59%). ¹H NMR (600 MHz, Methanol-d₄) δ 9.17 (s, 1H), 7.79 (s, 1H), 7.75 (dd, J=7.9, 1.7 Hz, 1H), 7.48 (d, J=8.0 Hz, 2H), 7.44-7.39 (m, 3H), 7.38-7.31 (m, 3H), 7.13 (s, 1H), 5.68 (t, J=8.0 Hz, 1H), 4.62-4.47 (m, 4H), 4.38-4.32 (m, 1H), 3.90 (d, J=11.1 Hz, 1H), 3.79 (dd, J=11.0, 4.0 Hz, 1H), 3.44-3.34 (m, 2H), 3.16-2.91 (m, 8H), 2.68-2.61 (m, 1H), 2.49 (s, 3H), 2.39-2.31 (m, 2H), 2.20 (dd, J=12.8, 8.0 Hz, 1H), 2.15-2.04 (m, 2H), 1.92-1.84 (m, 2H), 1.02 (s, 9H). HRMS m/z [M+H]⁺ calcd for C₄₉H₅₇N₈O₉S⁺ 933.3964, found 933.3954.

### Example 209

### Synthesis of LQ126-57

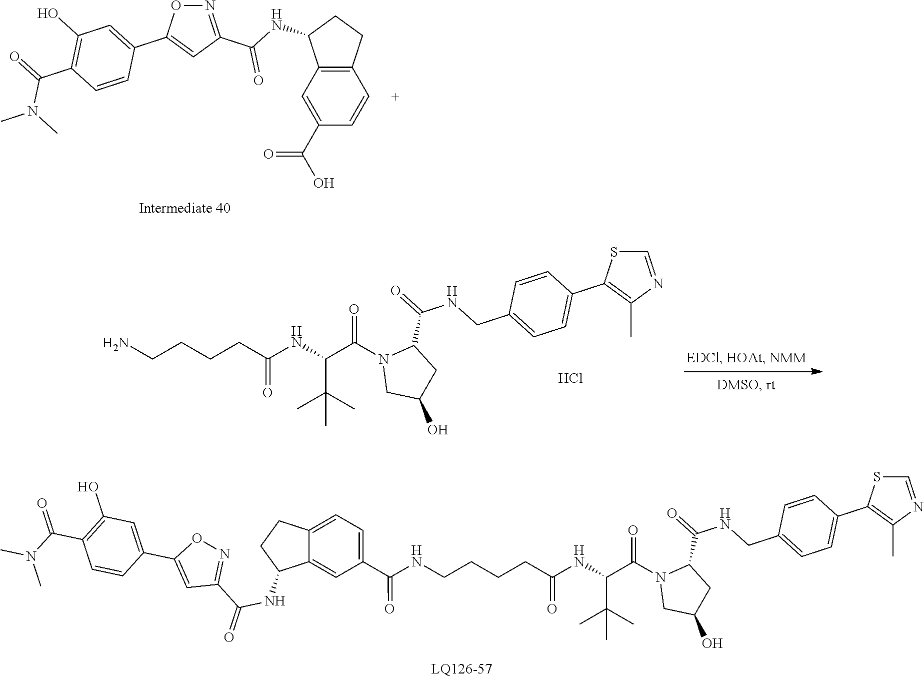

LQ126-57 was synthesized following the standard procedure for preparing LQ108-58 from intermediate 40 (5 mg, 0.01 mmol), (2S,4R)-1-((S)-2-(5-aminopentanamido)-3,3-dimethylbutanoyl)-4-hydroxy-N-(4-(4-methylthiazol-5-yl)benzyl)pyrrolidine-2-carboxamide (5.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-57 was obtained as white solid (6.1 mg, 64%). ¹H NMR (600 MHz, Methanol-d₄) δ 9.16 (s, 1H), 7.77 (s, 1H), 7.75-7.72 (m, 1H), 7.48 (d, J=8.2 Hz, 2H), 7.44-7.40 (m, 3H), 7.37-7.32 (m, 3H), 7.15 (s, 1H), 5.68 (t, J=7.9 Hz, 1H), 4.60 (s, 1H), 4.54 (dd, J=16.0, 8.4 Hz, 2H), 4.50-4.46 (m, 1H), 4.35 (d, J=15.6 Hz, 1H), 3.91-3.86 (m, 1H), 3.78 (dd, J=10.9, 3.9 Hz, 1H), 3.39-3.33 (m, 2H), 3.16-2.90 (m, 8H), 2.67-2.59 (m, 1H), 2.49 (s, 3H), 2.37-2.26 (m, 2H), 2.20 (dd, J=13.2, 7.7 Hz, 1H), 2.14-2.03 (m, 2H), 1.73-1.58 (m, 4H), 1.01 (s, 9H). HRMS m/z [M+H]⁺ calcd for C₅₀H₅₉N₈O₉S⁺ 947.4120, found 947.4125.

### Example 210

### Synthesis of LQ126-58

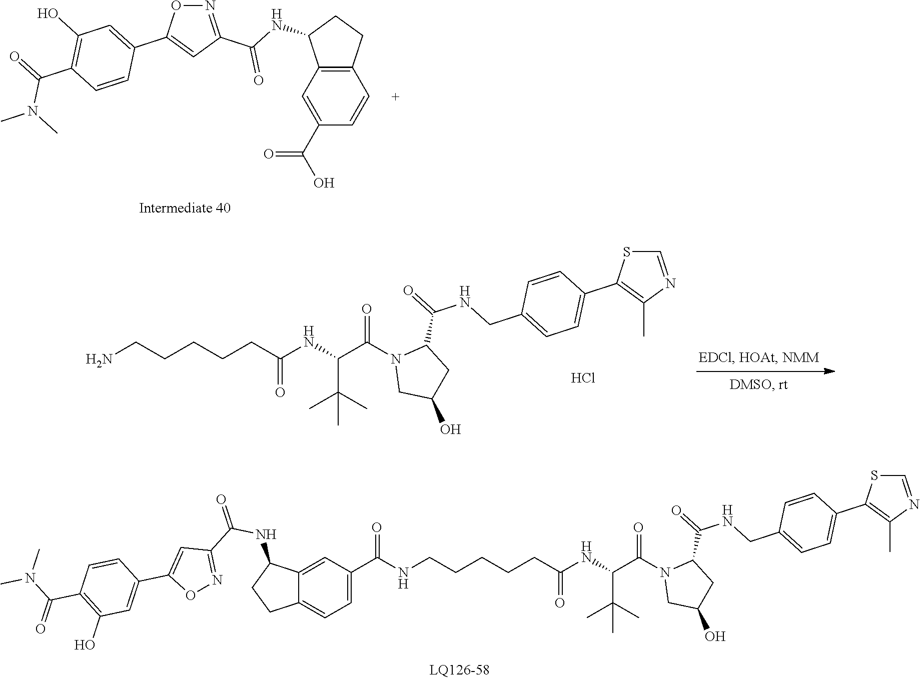

LQ126-58 was synthesized following the standard procedure for preparing LQ108-58 from intermediate 40 (5 mg, 0.01 mmol), (2S,4R)-1-((S)-2-(6-aminohexanamido)-3,3-dimethylbutanoyl)-4-hydroxy-N-(4-(4-methylthiazol-5-yl)benzyl)pyrrolidine-2-carboxamide (5.8 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-58 was obtained as white solid (6.1 mg, 63%). ¹H NMR (600 MHz, Methanol-d₄) δ 8.94 (s, 1H), 7.77 (s, 1H), 7.73 (dd, J=7.8, 1.8 Hz, 1H), 7.47-7.44 (m, 2H), 7.43-7.39 (m, 3H), 7.38-7.31 (m, 3H), 7.15 (s, 1H), 5.68 (t, J=7.9 Hz, 1H), 4.61 (s, 1H), 4.58-4.50 (m, 2H), 4.50-4.47 (m, 1H), 4.35 (d, J=15.5 Hz, 1H), 3.88 (d, J=11.0 Hz, 1H), 3.78 (dd, J=11.0, 3.9 Hz, 1H), 3.15-2.93 (m, 8H), 2.68-2.60 (m, 1H), 2.47 (s, 3H), 2.33-2.17 (m, 3H), 2.14-2.04 (m, 2H), 1.73-1.58 (m, 4H), 1.43-1.25 (m, 4H), 1.01 (s, 9H). HRMS m/z [M+H]⁺ calcd for C₅₁H₆₁N₈O₉S⁺ 961.4277, found 961.4275.

### Example 211

### Synthesis of LQ126-59

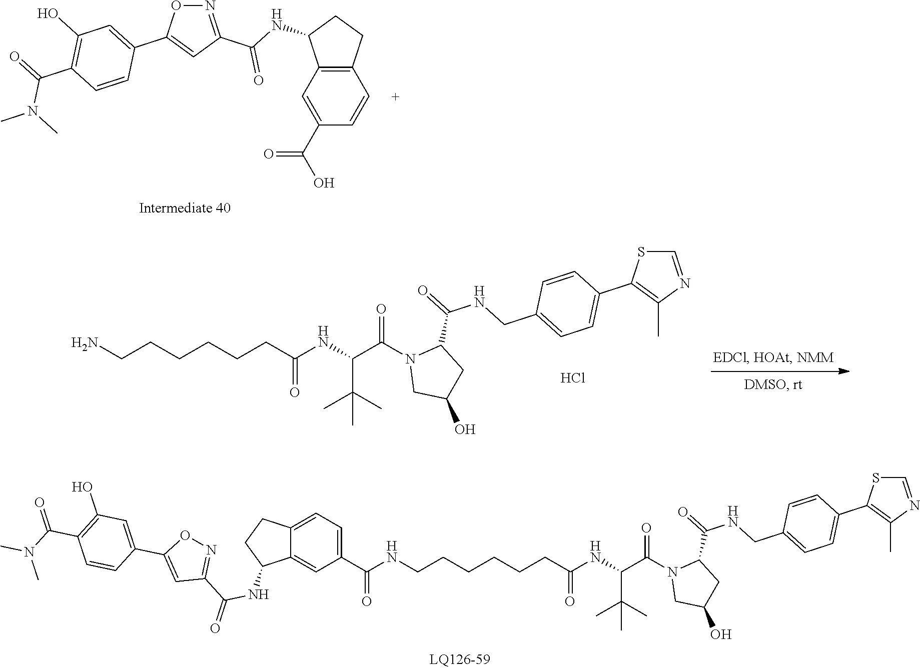

LQ126-59 was synthesized following the standard procedure for preparing LQ108-58 from intermediate 40 (5 mg, 0.01 mmol), (2S,4R)-1-((S)-2-(7-aminoheptanamido)-3,3-dimethylbutanoyl)-4-hydroxy-N-(4-(4-methylthiazol-5-yl)benzyl)pyrrolidine-2-carboxamide (5.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-59 was obtained as white solid (6.5 mg, 67%). ¹H NMR (600 MHz, Methanol-d₄) δ 9.10 (s, 1H), 7.76 (s, 1H), 7.73-7.71 (m, 1H), 7.47 (d, J=8.0 Hz, 2H), 7.44-7.39 (m, 3H), 7.37-7.31 (m, 3H), 7.15 (s, 1H), 5.68 (t, J=7.9 Hz, 1H), 4.61 (s, 1H), 4.59-4.47 (m, 3H), 4.35 (d, J=15.5 Hz, 1H), 3.92-3.87 (m, 1H), 3.79 (dd, J=10.9, 3.9 Hz, 1H), 3.37-3.32 (m, 2H), 3.16-2.92 (m, 8H), 2.67-2.60 (m, 1H), 2.48 (s, 3H), 2.32-2.18 (m, 3H), 2.15-2.03 (m, 2H), 1.65-1.56 (m, 4H), 1.42-1.32 (m, 4H), 1.02 (s, 9H). HRMS m/z [M+H]⁺ calcd for C₅₂H₆₃N₈O₉S⁺ 975.4433, found 975.4428.

### Example 212

### Synthesis of LQ126-60

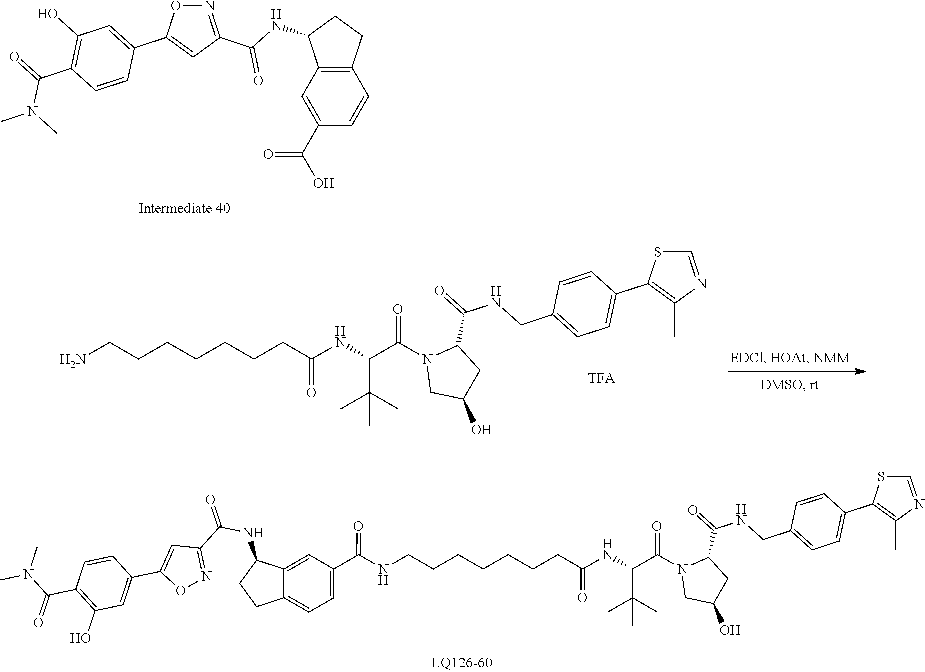

LQ126-60 was synthesized following the standard procedure for preparing LQ108-58 from intermediate 40 (5 mg, 0.01 mmol), (2S,4R)-1-((S)-2-(8-aminooctanamido)-3,3-dimethylbutanoyl)-4-hydroxy-N-(4-(4-methylthiazol-5-yl)benzyl)pyrrolidine-2-carboxamide (6.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-60 was obtained as white solid (7 mg, 71%). ¹H NMR (600 MHz, Methanol-d₄) δ 9.04 (s, 1H), 7.76 (s, 1H), 7.72 (dd, J=7.7, 1.7 Hz, 1H), 7.47 (d, J=8.0 Hz, 2H), 7.43-7.40 (m, 3H), 7.37-7.31 (m, 3H), 7.15 (s, 1H), 5.68 (t, J=7.9 Hz, 1H), 4.62 (s, 1H), 4.59-4.47 (m, 3H), 4.35 (d, J=15.5 Hz, 1H), 3.89 (d, J=10.9 Hz, 1H), 3.79 (dd, J=10.9, 3.9 Hz, 1H), 3.39-3.32 (m, 2H), 3.16-2.93 (m, 8H), 2.68-2.60 (m, 1H), 2.48 (s, 3H), 2.32-2.17 (m, 3H), 2.15-2.03 (m, 2H), 1.65-1.55 (m, 4H), 1.41-1.28 (m, 6H), 1.02 (s, 9H). HRMS m/z [M+H]⁺ calcd for C₅₃H₆₅N₈O₉S⁺ 989.4590, found 989.4589.

### Example 213

### Synthesis of LQ126-61

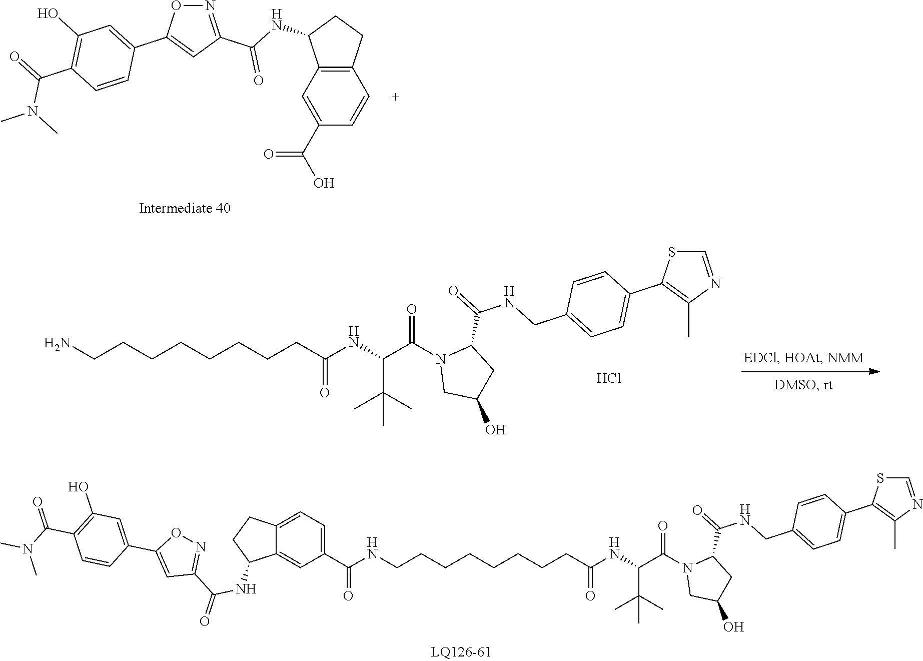 [SMILES: CN(C)C(=O)c1ccc(-c2cc(C(=O)N[C@@H]3CCc4ccc(C(=O)O)cc43)no2)cc1O ; Cc1ncsc1-c1ccc(CNC(=O)[C@@H]2C[C@@H](O)CN2C(=O)[C@@H](NC(=O)CCCCCCCCN)C(C)(C)C)cc1 ; Cl ; Cc1ncsc1-c1ccc(CNC(=O)[C@@H]2C[C@@H](O)CN2C(=O)[C@@H](NC(=O)CCCCCCCCNC(=O)c2ccc3c(c2)[C@H](NC(=O)c2cc(-c4ccc(C(=O)N(C)C)c(O)c4)on2)CC3)C(C)(C)C)cc1]

LQ126-61 was synthesized following the standard procedure for preparing LQ108-58 from intermediate 40 (5 mg, 0.01 mmol), (2S,4R)-1-((S)-2-(9-aminononanamido)-3,3-dimethylbutanoyl)-4-hydroxy-N-(4-(4-methylthiazol-5-yl)benzyl)pyrrolidine-2-carboxamide (6.2 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-61 was obtained as white solid (6.5 mg, 65%). ¹H NMR (600 MHz, Methanol-d₄) δ 9.04 (s, 1H), 7.76 (s, 1H), 7.72 (dd, J=7.9, 1.7 Hz, 1H), 7.49-7.45 (m, 2H), 7.44-7.40 (m, 3H), 7.37-7.32 (m, 3H), 7.16 (s, 1H), 5.68 (t, J=7.9 Hz, 1H), 4.62 (s, 1H), 4.59-4.47 (m, 3H), 4.35 (d, J=15.5 Hz, 1H), 3.92-3.87 (m, 1H), 3.79 (dd, J=10.9, 3.9 Hz, 1H), 3.37-3.32 (m, 2H), 3.18-2.92 (m, 8H), 2.68-2.60 (m, 1H), 2.48 (s, 3H), 2.32-2.18 (m, 3H), 2.16-2.03 (m, 2H), 1.65-1.54 (m, 4H), 1.40-1.28 (m, 8H), 1.02 (s, 9H). HRMS m/z [M+H]⁺ calcd for C₅₄H₆₇N₈O₉S⁺ 1003.4746, found 1003.4752.

### Example 214

### Synthesis of LQ126-62

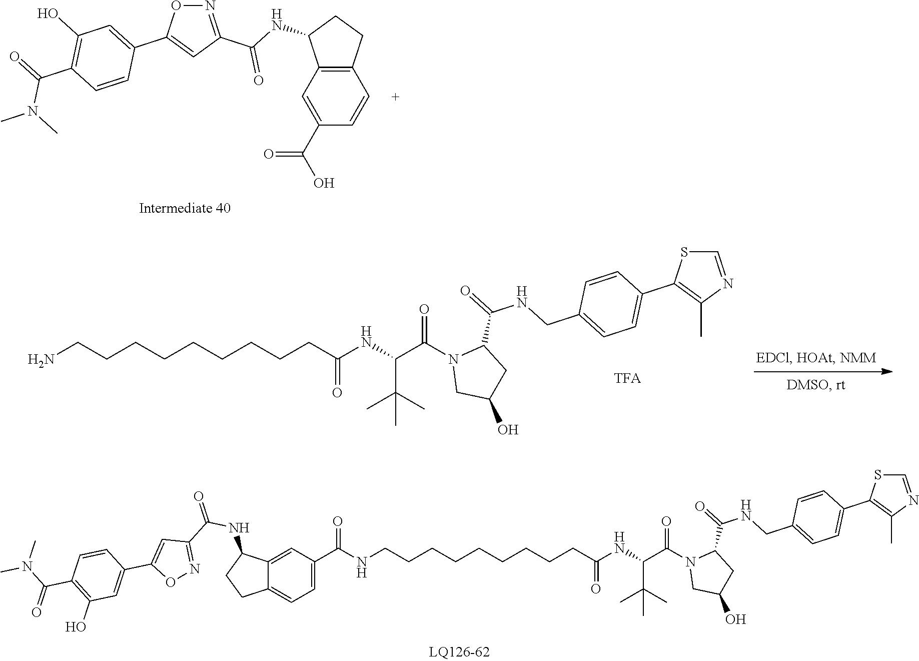

LQ126-62 was synthesized following the standard procedure for preparing LQ108-58 from intermediate 40 (5 mg, 0.01 mmol), (2S,4R)-1-((S)-2-(10-aminodecanamido)-3,3-dimethylbutanoyl)-4-hydroxy-N-(4-(4-methylthiazol-5-yl)benzyl)pyrrolidine-2-carboxamide (6.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-62 was obtained as white solid (6.8 mg, 67%). ¹H NMR (600 MHz, Methanol-d₄) δ 9.03 (s, 1H), 7.76 (s, 1H), 7.74-7.69 (m, 1H), 7.47 (d, J=8.2 Hz, 2H), 7.44-7.39 (m, 3H), 7.37-7.32 (m, 3H), 7.15 (s, 1H), 5.68 (t, J=7.9 Hz, 1H), 4.62 (s, 1H), 4.59-4.52 (m, 2H), 4.50-4.47 (m, 1H), 4.35 (d, J=15.5 Hz, 1H), 3.89 (d, J=10.9 Hz, 1H), 3.79 (dd, J=11.0, 3.9 Hz, 1H), 3.36-3.31 (m, 2H), 3.15-2.93 (m, 8H), 2.68-2.61 (m, 1H), 2.48 (s, 3H), 2.30-2.18 (m, 3H), 2.15-2.03 (m, 2H), 1.62-1.53 (m, 4H), 1.39-1.26 (m, 10H), 1.02 (s, 9H). HRMS m/z [M+H]⁺ calcd for C₅₅H₆₉N₈O₉S⁺ 1017.4903, found 1017.4900.

### Example 215

### Synthesis of LQ126-63

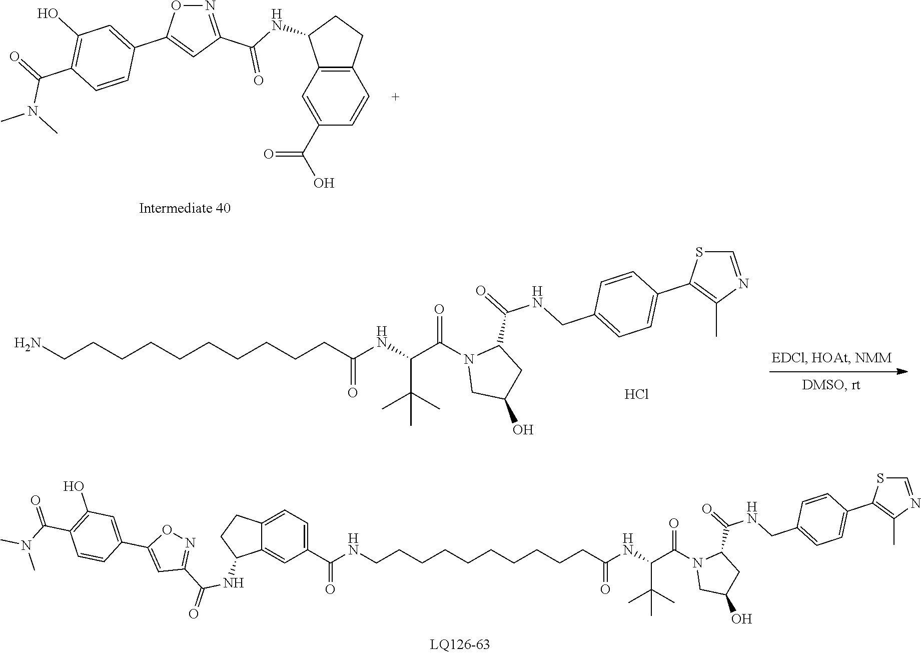

LQ126-63 was synthesized following the standard procedure for preparing LQ108-58 from intermediate 40 (5 mg, 0.01 mmol), (2S,4R)-1-((S)-2-(11-aminoundecanamido)-3,3-dimethylbutanoyl)-4-hydroxy-N-(4-(4-methylthiazol-5-yl)benzyl)pyrrolidine-2-carboxamide (6.5 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-63 was obtained as white solid (6.3 mg, 61%). ¹H NMR (600 MHz, Methanol-d₄) δ 9.15 (s, 1H), 7.76 (s, 1H), 7.72 (dd, J=8.0, 1.7 Hz, 1H), 7.50-7.46 (m, 2H), 7.45-7.41 (m, 3H), 7.39-7.32 (m, 3H), 7.15 (s, 1H), 5.68 (t, J=7.9 Hz, 1H), 4.62 (s, 1H), 4.59-4.52 (m, 2H), 4.51-4.47 (m, 1H), 4.36 (d, J=15.5 Hz, 1H), 3.93-3.87 (m, 1H), 3.80 (dd, J=11.0, 3.9 Hz, 1H), 3.36-3.32 (m, 2H), 3.17-2.91 (m, 8H), 2.68-2.60 (m, 1H), 2.50 (s, 3H), 2.31-2.18 (m, 3H), 2.16-2.01 (m, 2H), 1.63-1.52 (m, 4H), 1.40-1.25 (m, 12H), 1.02 (s, 9H). HRMS m/z [M+H]⁺ calcd for C₅₆H₇₁N₈O₉S⁺ 1031.5059, found 1031.5056.

### Example 216

### Synthesis of LQ126-77

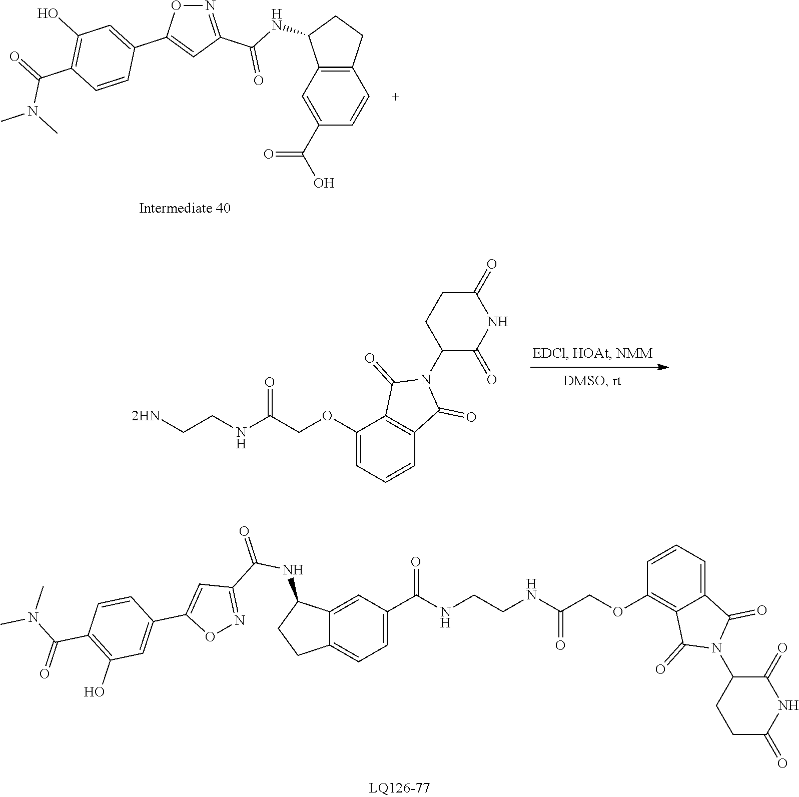

LQ126-77 was synthesized following the standard procedure for preparing LQ108-58 from intermediate 40 (5 mg, 0.01 mmol), N-(2-aminoethyl)-2-((2-(2,6-dioxopiperidin-3-yl)-1,3-dioxoisoindolin-4-yl)oxy)acetamide (4.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-77 was obtained as white solid (4.6 mg, 58%). ¹H NMR (600 MHz, Methanol-d₄) δ 7.73 (d, J=8.0 Hz, 1H), 7.71-7.64 (m, 2H), 7.44-7.29 (m, 6H), 7.10 (d, J=9.6 Hz, 1H), 5.71-5.64 (m, 1H), 5.02 (dd, J=12.9, 5.4 Hz, 1H), 4.79-4.72 (m, 2H), 3.63-3.50 (m, 4H), 3.20-2.93 (m, 8H), 2.86-2.76 (m, 1H), 2.71-2.54 (m, 3H), 2.18-2.03 (m, 2H). HRMS m/z [M+H]⁺ calcd for C₄₀H₃₈N₇O₁₁⁺ 792.2624, found 792.2614.

### Example 217

### Synthesis of LQ126-78

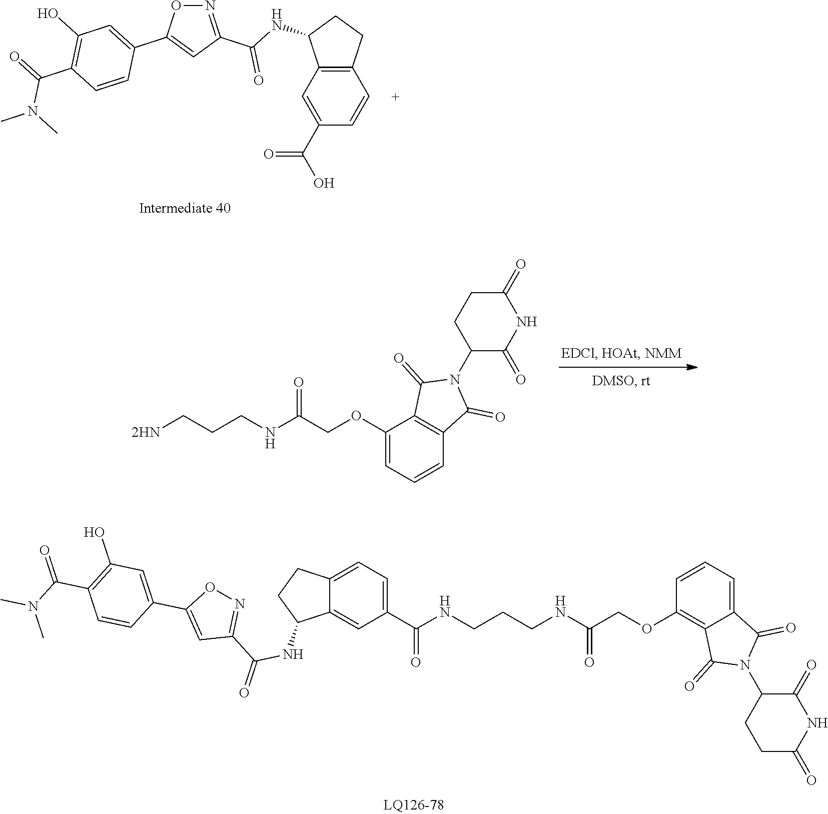

LQ126-78 was synthesized following the standard procedure for preparing LQ108-58 from intermediate 40 (5 mg, 0.01 mmol), N-(3-aminopropyl)-2-((2-(2,6-dioxopiperidin-3-yl)-1,3-dioxoisoindolin-4-yl)oxy)acetamide (5 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-78 was obtained as white solid (5.1 mg, 63%). ¹H NMR (600 MHz, Methanol-d₄) δ 7.82-7.75 (m, 2H), 7.74-7.68 (m, 1H), 7.54-7.49 (m, 1H), 7.45-7.39 (m, 2H), 7.37-7.29 (m, 3H), 7.17 (s, 1H), 5.68-5.62 (m, 1H), 5.14-5.07 (m, 1H), 4.77-4.74 (m, 2H), 3.51-3.39 (m, 4H), 3.19-2.92 (m, 8H), 2.89-2.80 (m, 1H), 2.78-2.60 (m, 3H), 2.17-2.08 (m, 2H), 1.92-1.84 (m, 2H). HRMS m/z [M+H]⁺ calcd for C₄₁H₄₀N₇O₁₁⁺ 806.2780, found 806.2762.

### Example 218

### Synthesis of LQ126-79

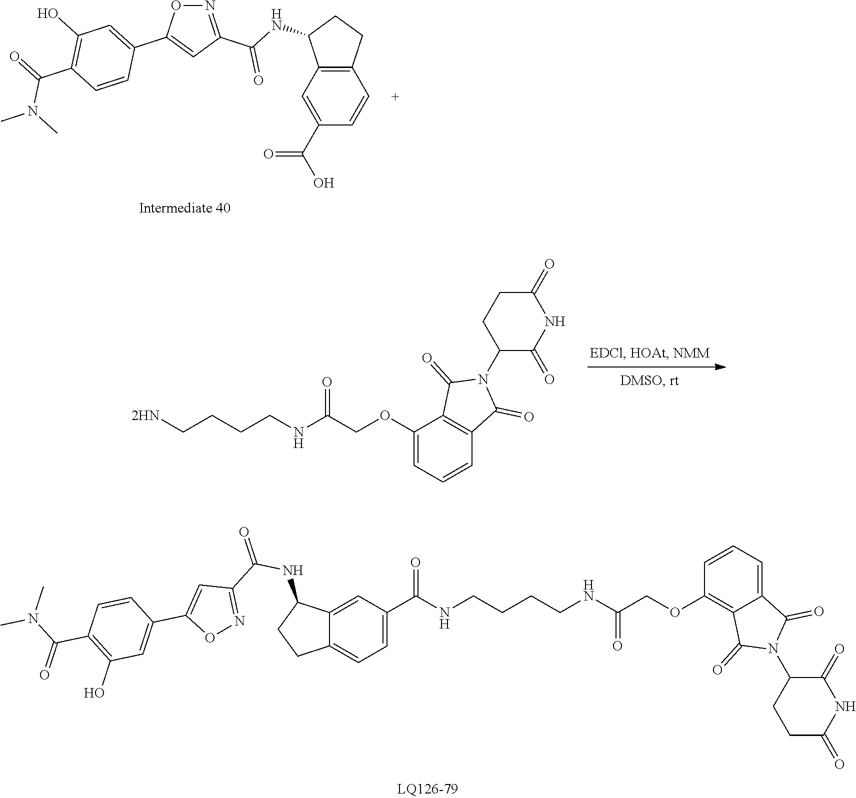

LQ126-79 was synthesized following the standard procedure for preparing LQ108-58 from intermediate 40 (5 mg, 0.01 mmol), N-(4-aminobutyl)-2-((2-(2,6-dioxopiperidin-3-yl)-1,3-dioxoisoindolin-4-yl)oxy)acetamide (5.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-79 was obtained as white solid (5.8 mg, 71%). ¹H NMR (600 MHz, Methanol-d₄) δ 7.80-7.69 (m, 3H), 7.49 (t, J=7.2 Hz, 1H), 7.44-7.30 (m, 5H), 7.15 (d, J=8.5 Hz, 1H), 5.70 (t, J=8.0 Hz, 1H), 5.09 (dd, J=12.7, 5.4 Hz, 1H), 4.76-4.68 (m, 2H), 3.50-3.35 (m, 4H), 3.18-2.94 (m, 8H), 2.85-2.62 (m, 4H), 2.18-2.04 (m, 2H), 1.76-1.59 (m, 4H). HRMS m/z [M+H]⁺ calcd for C₄₂H₄₂N₇O₁₁⁺ 820.2937, found 820.2929.

### Example 219

### Synthesis of LQ126-80

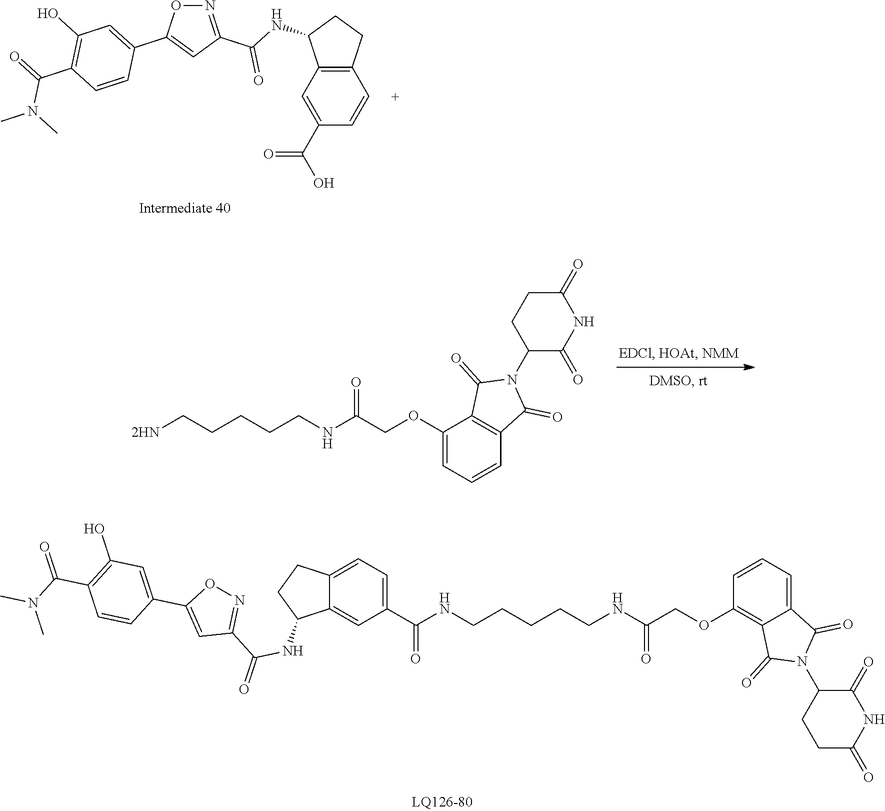

LQ126-80 was synthesized following the standard procedure for preparing LQ108-58 from intermediate 40 (5 mg, 0.01 mmol), N-(5-aminopentyl)-2-((2-(2,6-dioxopiperidin-3-yl)-1,3-dioxoisoindolin-4-yl)oxy)acetamide (5.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-80 was obtained as white solid (5 mg, 69%). ¹H NMR (600 MHz, Methanol-d₄) δ 7.81-7.73 (m, 2H), 7.68 (ddd, J=17.9, 7.9, 1.7 Hz, 1H), 7.49 (dd, J=11.0, 7.3 Hz, 1H), 7.44-7.39 (m, 2H), 7.38-7.33 (m, 2H), 7.29 (dd, J=14.8, 7.9 Hz, 1H), 7.16 (s, 1H), 5.69-5.63 (m, 1H), 5.16-5.07 (m, 1H), 4.75-4.70 (m, 2H), 3.43-3.34 (m, 3H), 3.19-2.93 (m, 8H), 2.91-2.82 (m, 1H), 2.78-2.60 (m, 3H), 2.18-2.09 (m, 2H), 1.69-1.61 (m, 4H), 1.51-1.44 (m, 2H). HRMS m/z [M+H]⁺ calcd for C₄₃H₄₄N₇O₁₁⁺ 834.3093, found 834.3091.

### Example 220

### Synthesis of LQ126-81

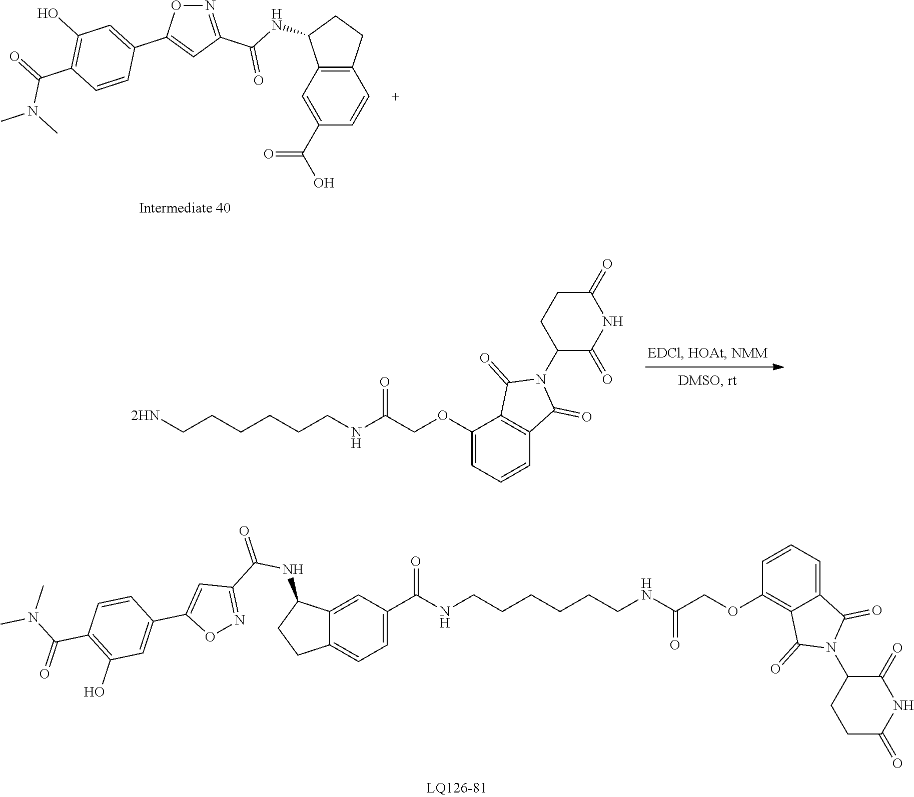

LQ126-81 was synthesized following the standard procedure for preparing LQ108-58 from intermediate 40 (5 mg, 0.01 mmol), N-(6-aminohexyl)-2-((2-(2,6-dioxopiperidin-3-yl)-1,3-dioxoisoindolin-4-yl)oxy)acetamide (5.4 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-81 was obtained as white solid (5.5 mg, 65%). ¹H NMR (600 MHz, Methanol-d₄) δ 7.82-7.75 (m, 2H), 7.75-7.69 (m, 1H), 7.50 (dd, J=7.3, 4.4 Hz, 1H), 7.44-7.32 (m, 5H), 7.16 (s, 1H), 5.70 (t, J=7.9 Hz, 1H), 5.15-5.06 (m, 1H), 4.76-4.71 (m, 2H), 3.38-3.34 (m, 3H), 3.17-2.94 (m, 8H), 2.90-2.81 (m, 1H), 2.76-2.62 (m, 3H), 2.16-2.10 (m, 2H), 1.65-1.57 (m, 4H), 1.47-1.39 (m, 4H). HRMS m/z [M+H]⁺ calcd for C₄₄H₄₆N₇O₁₁⁺ 848.3250, found 848.3160.

### Example 221

### Synthesis of LQ126-82

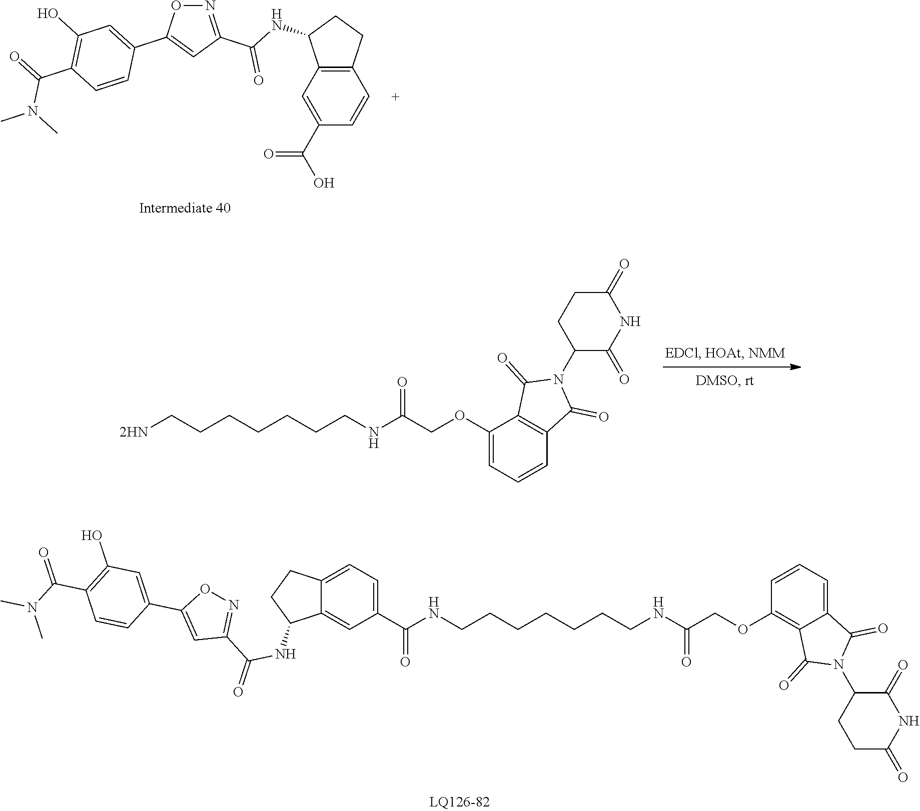

LQ126-82 was synthesized following the standard procedure for preparing LQ108-58 from intermediate 40 (5 mg, 0.01 mmol), N-(7-aminoheptyl)-2-((2-(2,6-dioxopiperidin-3-yl)-1,3-dioxoisoindolin-4-yl)oxy)acetamide (5.4 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-82 was obtained as white solid (6.2 mg, 72%). ¹H NMR (600 MHz, Methanol-d₄) δ 7.81-7.76 (m, 2H), 7.73-7.68 (m, 1H), 7.48 (dd, J=8.9, 7.3 Hz, 1H), 7.44-7.38 (m, 2H), 7.37-7.32 (m, 3H), 7.16 (d, J=4.3 Hz, 1H), 5.69 (t, J=8.0 Hz, 1H), 5.16-5.11 (m, 1H), 4.76-4.71 (m, 2H), 3.32-3.28 (m, 3H), 3.18-2.94 (m, 8H), 2.91-2.82 (m, 1H), 2.79-2.63 (m, 3H), 2.19-2.10 (m, 2H), 1.63-1.54 (m, 4H), 1.43-1.34 (m, 6H). HRMS m/z [M+H]⁺ calcd for C₄₅H₄₈N₇O₁₁⁺ 862.3406, found 862.3405.

### Example 222

### Synthesis of LQ126-83

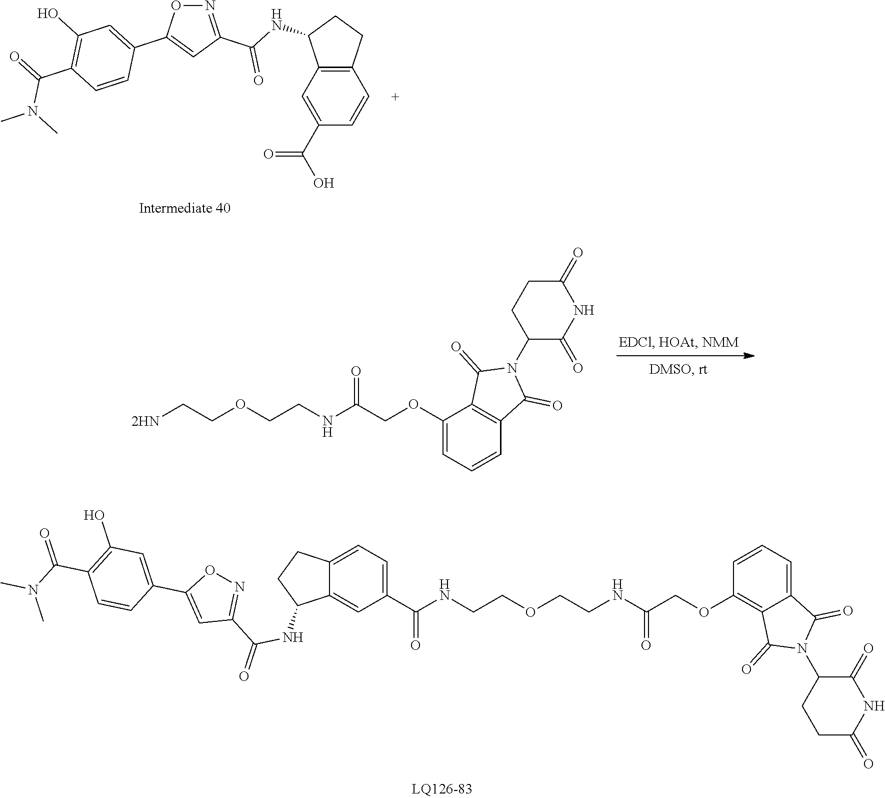

LQ126-83 was synthesized following the standard procedure for preparing LQ108-58 from intermediate 40 (5 mg, 0.01 mmol), N-(2-(2-aminoethoxy)ethyl)-2-((2-(2,6-dioxopiperidin-3-yl)-1,3-dioxoisoindolin-4-yl)oxy)acetamide (5.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-83 was obtained as white solid (5 mg, 60%). ¹H NMR (600 MHz, Methanol-d₄) δ 7.80-7.75 (m, 2H), 7.70-7.67 (m, 1H), 7.48 (d, J=7.3 Hz, 1H), 7.40 (dd, J=7.9, 1.6 Hz, 1H), 7.37-7.31 (m, 3H), 7.21 (d, J=7.9 Hz, 1H), 7.13 (s, 1H), 5.58 (t, J=7.9 Hz, 1H), 5.04 (dd, J=12.6, 5.5 Hz, 1H), 4.73-4.61 (m, 2H), 3.73-3.59 (m, 5H), 3.57-3.49 (m, 3H), 3.20-2.96 (m, 8H), 2.92-2.85 (m, 1H), 2.80-2.56 (m, 4H), 2.16-2.04 (m, 1H). HRMS m/z [M+H]⁺ calcd for C₄₂H₄₂N₇O₁₂⁺ 836.2886, found 836.2879.

### Example 223

### Synthesis of LQ126-84

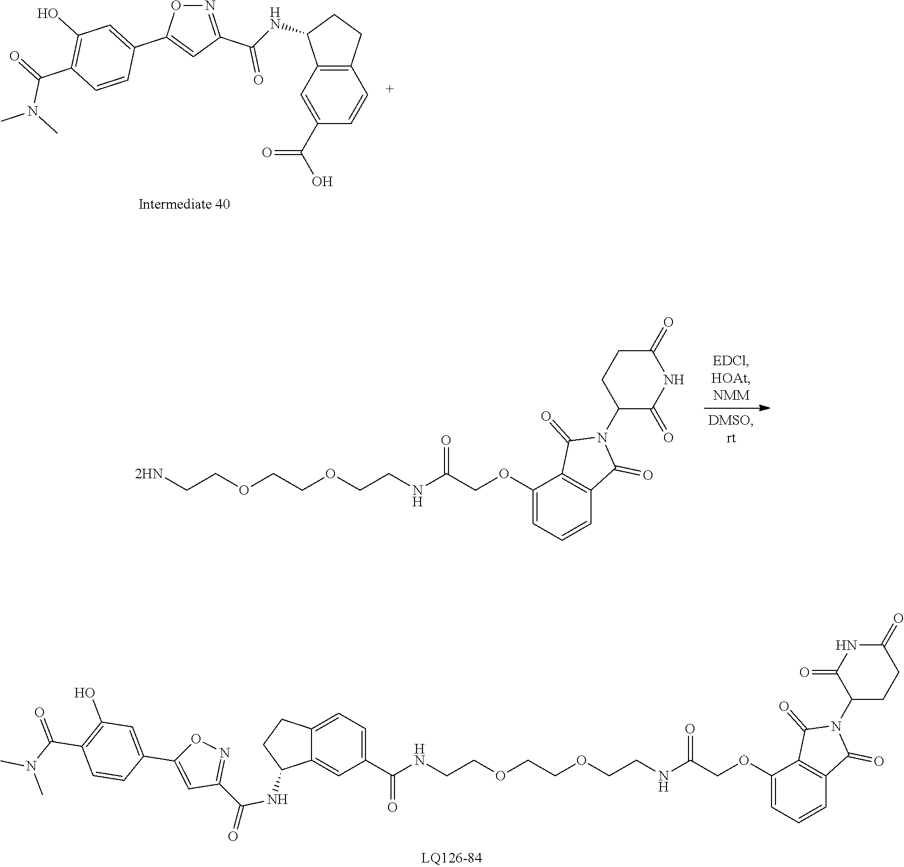

LQ126-84 was synthesized following the standard procedure for preparing LQ108-58 from intermediate 40 (5 mg, 0.01 mmol), N-(2-(2-(2-aminoethoxy)ethoxy)ethyl)-2-((2-(2,6-dioxopiperidin-3-yl)-1,3-dioxoisoindolin-4-yl)oxy)acetamide (5.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-84 was obtained as white solid (5.6 mg, 64%). ¹H NMR (600 MHz, Methanol-d₄) δ 7.80 (d, J=7.3 Hz, 1H), 7.78-7.74 (m, 1H), 7.74-7.69 (m, 1H), 7.45 (t, J=7.5 Hz, 1H), 7.42-7.39 (m, 1H), 7.38-7.31 (m, 4H), 7.14 (d, J=5.1 Hz, 1H), 5.68 (t, J=8.0 Hz, 1H), 5.11 (dd, J=12.8, 5.5 Hz, 1H), 4.70-4.67 (m, 2H), 3.66-3.57 (m, 8H), 3.55-3.51 (m, 2H), 3.49-3.39 (m, 2H), 3.18-2.93 (m, 8H), 2.90-2.82 (m, 1H), 2.79-2.70 (m, 3H), 2.69-2.62 (m, 1H), 2.19-2.09 (m, 1H). HRMS m/z [M+H]⁺ calcd for C₄₄H₄₆N₇O₁₃⁺ 880.3148, found 880.3122.

### Example 224

### Synthesis of LQ126-85

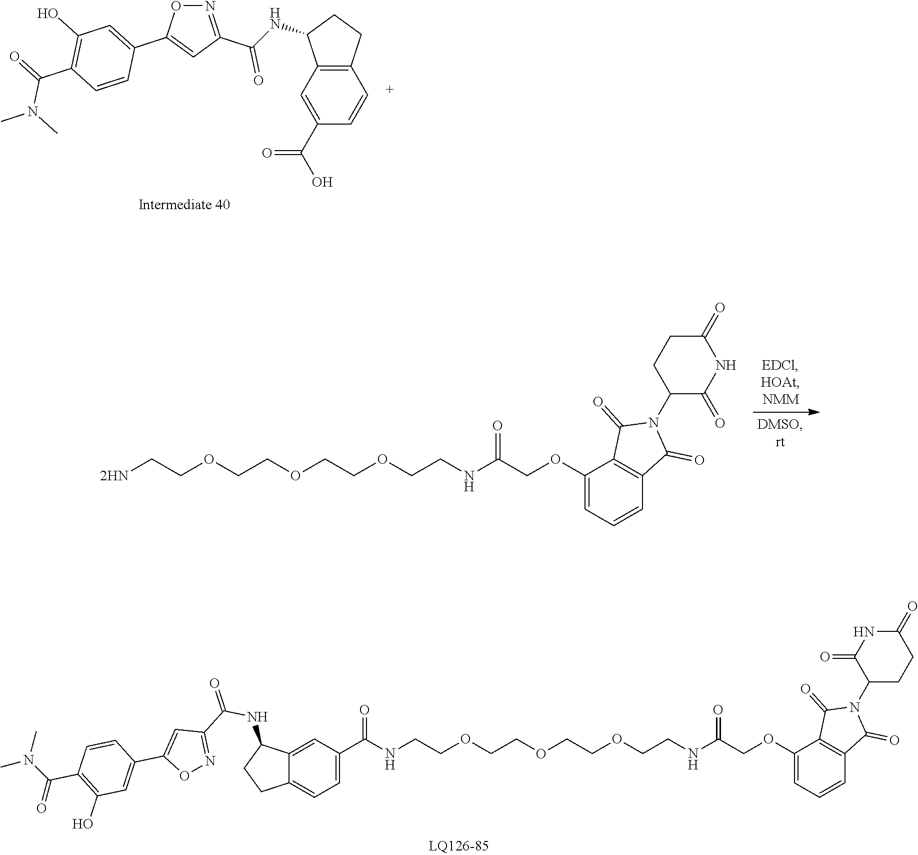

LQ126-85 was synthesized following the standard procedure for preparing LQ108-58 from intermediate 40 (5 mg, 0.01 mmol), N-(2-(2-(2-(2-aminoethoxy)ethoxy)ethoxy)ethyl)-2-((2-(2,6-dioxopiperidin-3-yl)-1,3-dioxoisoindolin-4-yl)oxy)acetamide (6.2 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-85 was obtained as white solid (6.2 mg, 67%). ¹H NMR (600 MHz, Methanol-d₄) δ 7.79 (d, J=3.9 Hz, 1H), 7.77-7.74 (m, 1H), 7.74-7.70 (m, 1H), 7.46 (dd, J=7.3, 3.3 Hz, 1H), 7.41-7.36 (m, 2H), 7.36-7.30 (m, 3H), 7.13 (d, J=4.6 Hz, 1H), 5.67 (t, J=8.0 Hz, 1H), 5.13-5.07 (m, 1H), 4.71 (s, 2H), 3.63-3.50 (m, 14H), 3.44-3.39 (m, 2H), 3.15-2.93 (m, 8H), 2.89-2.81 (m, 1H), 2.77-2.60 (m, 3H), 2.16-2.07 (m, 2H). HRMS m/z [M+H]⁺ calcd for C₄₆H₅₀N₇O₁₄⁺ 924.3410, found 924.3384.

### Example 225

### Synthesis of LQ126-86

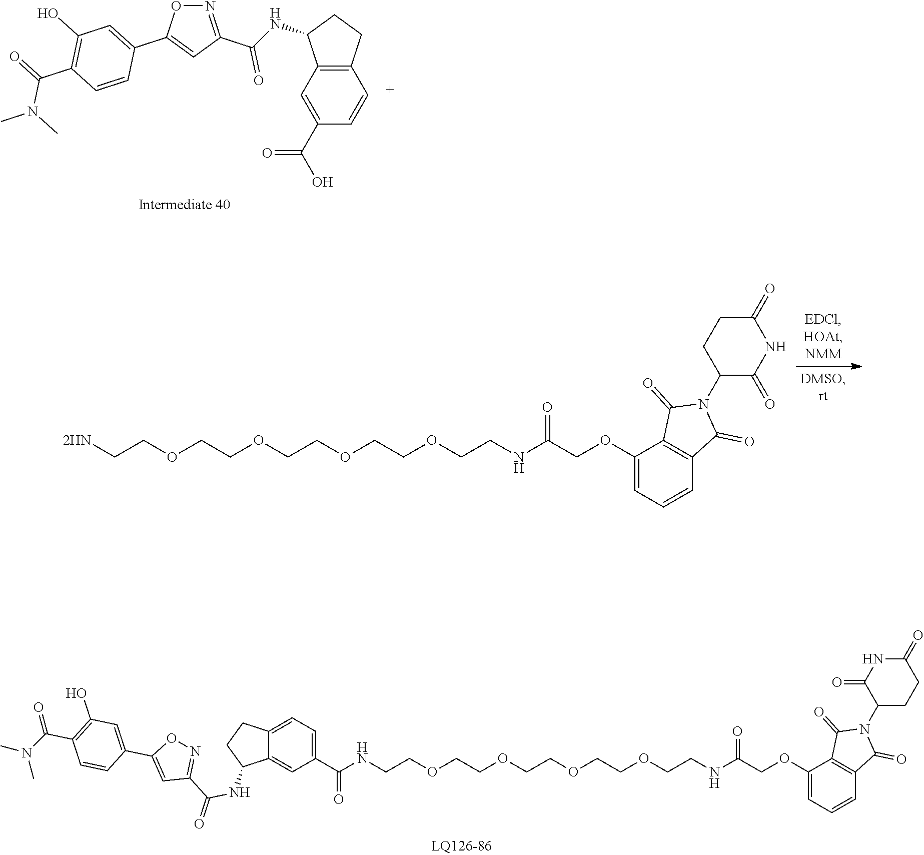

LQ126-86 was synthesized following the standard procedure for preparing LQ108-58 from intermediate 40 (5 mg, 0.01 mmol), N-(14-amino-3,6,9,12-tetraoxatetradecyl)-2-((2-(2,6-dioxopiperidin-3-yl)-1,3-dioxoisoindolin-4-yl)oxy)acetamide (6.6 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-86 was obtained as white solid (6.7 mg, 70%). ¹H NMR (600 MHz, Methanol-d₄) δ 7.82 (d, J=4.6 Hz, 1H), 7.78 (dd, J=8.4, 7.3 Hz, 1H), 7.77-7.73 (m, 1H), 7.50 (dd, J=7.3, 1.9 Hz, 1H), 7.44-7.39 (m, 2H), 7.38-7.33 (m, 3H), 7.16 (d, J=2.3 Hz, 1H), 5.70 (t, J=7.9 Hz, 1H), 5.13 (dd, J=12.8, 5.5 Hz, 1H), 4.76-4.73 (m, 2H), 3.66-3.53 (m, 18H), 3.47 (t, J=5.3 Hz, 2H), 3.17-2.95 (m, 8H), 2.92-2.84 (m, 1H), 2.79-2.62 (m, 3H), 2.18-2.09 (m, 2H). HRMS m/z [M+H]⁺ calcd for C₄₈H₅₄N₇O₁₅⁺ 968.3672, found 968.3661.

### Example 226

### Synthesis of LQ126-87

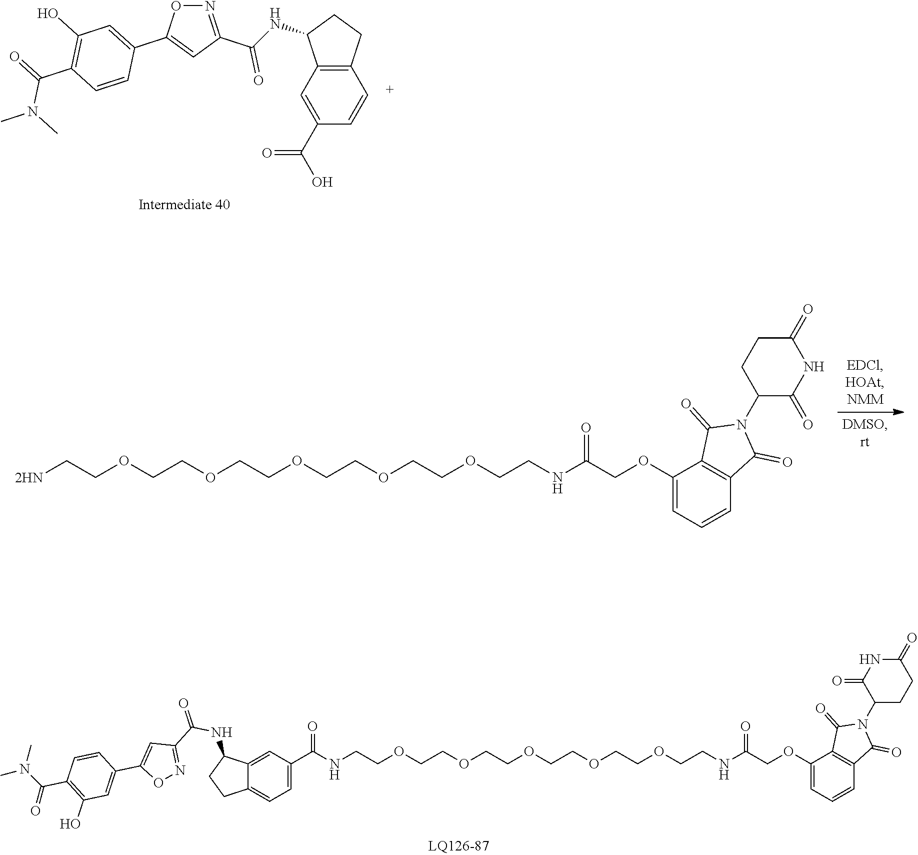

LQ126-87 was synthesized following the standard procedure for preparing LQ108-58 from intermediate 40 (5 mg, 0.01 mmol), N-(17-amino-3,6,9,12,15-pentaoxaheptadecyl)-2-((2-(2,6-dioxopiperidin-3-yl)-1,3-dioxoisoindolin-4-yl)oxy)acetamide (7.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-87 was obtained as white solid (6.1 mg, 61%). ¹H NMR (600 MHz, Methanol-d₄) δ 7.82 (d, J=3.8 Hz, 1H), 7.78 (dd, J=8.5, 7.3 Hz, 1H), 7.77-7.74 (m, 1H), 7.50 (dd, J=7.3, 2.3 Hz, 1H), 7.44-7.39 (m, 2H), 7.38-7.33 (m, 3H), 7.16 (d, J=1.8 Hz, 1H), 5.70 (t, J=8.0 Hz, 1H), 5.16-5.11 (m, 1H), 4.76-4.73 (m, 2H), 3.69-3.51 (m, 22H), 3.49 (t, J=5.3 Hz, 2H), 3.18-2.95 (m, 8H), 2.92-2.84 (m, 1H), 2.80-2.71 (m, 2H), 2.70-2.62 (m, 1H), 2.19-2.09 (m, 2H). HRMS m/z [M+H]⁺ calcd for C₅₀H₅₈N₇O₁₆⁺ 1012.3935, found 1012.3928.

### Example 227

### Synthesis of Intermediate 46

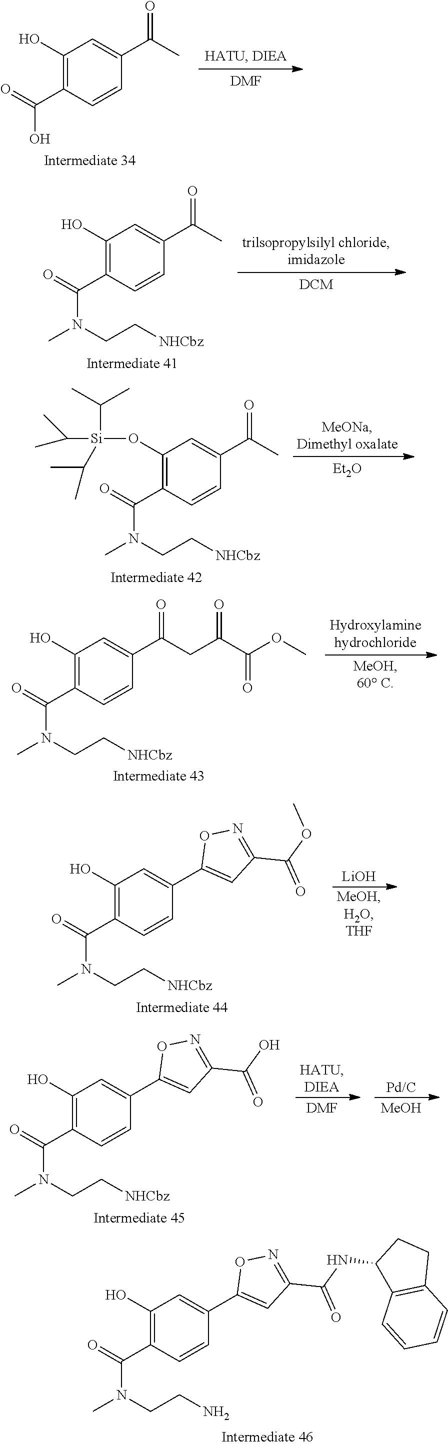

### Intermediate 41: Benzyl (2-(4-acetyl-2-hydroxy-N-methylbenzamido)ethyl)carbamate

Intermediate 41 was synthesized according to the procedures for the preparation of intermediate 3 as a white solid in 52% yield. MS (ESI): m/z 371.4 [M+H]⁺.

### Intermediate 42: Benzyl (2-(4-acetyl-N-methyl-2-((triisopropylsilyl)oxy)benzamido)ethyl)carbamate

Intermediate 42 was synthesized according to the procedures for the preparation of intermediate 36 as a yellow oil in 64% yield. MS (ESI): m/z 527.5 [M+H]⁺.

### Intermediate 43: Methyl 4-(4-((2-(((benzyloxy)carbonyl)amino)ethyl)(methyl)carbamoyl)-3-hydroxyphenyl)-2,4-dioxobutanoate

Intermediate 43 was synthesized according to the procedures for the preparation of intermediate 37 as a yellow solid in 16% yield. MS (ESI): m/z 457.3 [M+H]⁺.

### Intermediate 44: methyl 5-(4-((2-(((benzyloxy)carbonyl)amino)ethyl)(methyl)carbamoyl)-3-hydroxyphenyl)isoxazole-3-carboxylate

Intermediate 44 was synthesized according to the procedures for the preparation of intermediate 38 as a white solid in 62% yield. MS (ESI): m/z 454.4 [M+H]⁺.

### Intermediate 45: 5-(4-((2-(((benzyloxy)carbonyl)amino)ethyl)(methyl)carbamoyl)-3-hydroxyphenyl)isoxazole-3-carboxylic acid

Intermediate 45 was synthesized according to the procedures for the preparation of intermediate 4 as a white solid in 17% yield. MS (ESI): m/z 440.6 [M+H]⁺.

### Intermediate 46: (R)-5-(4-((2-aminoethyl)(methyl)carbamoyl)-3-hydroxyphenyl)-N-(2,3-dihydro-1H-inden-1-yl)isoxazole-3-carboxamide

Intermediate 46 was synthesized according to the procedures for the preparation of intermediate 3 as a white solid in 44% yield. MS (ESI): m/z 421.5 [M+H]⁺.

### Example 228

### Synthesis of LQ126-89

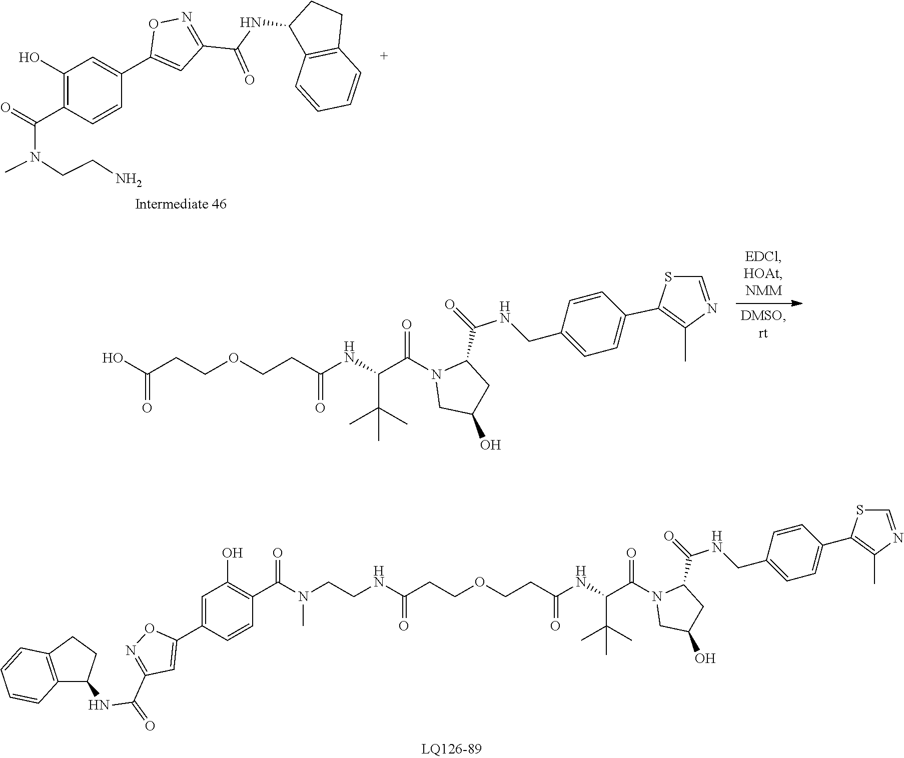 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)CCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCC(=O)NCCN(C)C(=O)c2ccc(-c3cc(C(=O)N[C@@H]4CCc5ccccc54)no3)cc2O)C(C)(C)C)cc1]

To a solution of Intermediate 46 (4 mg, 0.01 mmol) in DMSO (1 mL) were added 3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoic acid (5.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv). After being stirred overnight at room temperature, the resulting mixture was purified by preparative HPLC (5%-70% acetonitrile/0.1% TFA in H₂O) to afford LQ126-89 as white solid (8.7 mg, 78%). ¹H NMR (600 MHz, Methanol-d₄) δ 9.01 (s, 1H), 7.48-7.39 (m, 5H), 7.38-7.31 (m, 3H), 7.29 (d, J=7.4 Hz, 1H), 7.27-7.20 (m, 2H), 7.15 (s, 1H), 5.65 (t, J=7.7 Hz, 1H), 4.70-4.58 (m, 2H), 4.57-4.48 (m, 2H), 4.36 (t, J=18.5 Hz, 1H), 3.92 (d, J=11.0 Hz, 1H), 3.82 (dd, J=10.9, 3.8 Hz, 1H), 3.79-3.59 (m, 8H), 3.56-3.41 (m, 2H), 3.16-3.07 (m, 3H), 3.03-2.91 (m, 4H), 2.65-2.57 (m, 1H), 2.49 (s, 3H), 2.44-2.37 (m, 1H), 2.28-2.22 (m, 1H), 2.13-2.03 (m, 2H), 1.06 (s, 9H). HRMS m/z [M+H]⁺ calcd for C₅₁H₆₁N₈O₁₀S⁺ 977.4226, found 977.4237.

### Example 229

### Synthesis of LQ126-90

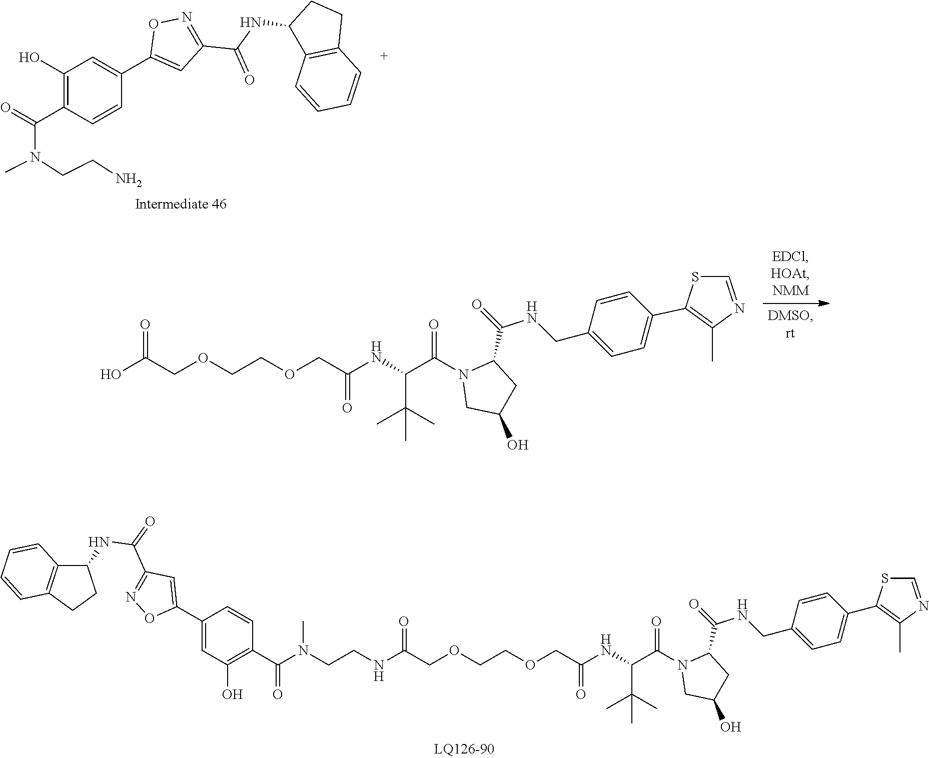 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)COCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCC(=O)NCCN(C)C(=O)c2ccc(-c3cc(C(=O)N[C@@H]4CCc5ccccc54)no3)cc2O)C(C)(C)C)cc1]

LQ126-90 was synthesized following the standard procedure for preparing LQ126-89 from intermediate 46 (4 mg, 0.01 mmol), 2-(2-(2-(((S)-1-((2S,4R)-4-hydroxy-2-(3-(4-(4-methylthiazol-5-yl)phenyl)propanoyl)pyrrolidin-1-yl)-3,3-dimethyl-1-oxobutan-2-yl)amino)-2-oxoethoxy)ethoxy)acetic acid (5.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-90 was obtained as white solid (7.4 mg, 75%). ¹H NMR (600 MHz, Methanol-d₄) δ 8.99 (s, 1H), 7.46-7.38 (m, 5H), 7.35-7.18 (m, 6H), 7.12 (s, 1H), 5.63 (t, J=7.5 Hz, 1H), 4.76-4.68 (m, 1H), 4.64-4.56 (m, 1H), 4.51-4.48 (m, 2H), 4.36 (d, J=15.4 Hz, 1H), 4.12-3.98 (m, 4H), 3.92-3.68 (m, 6H), 3.58-3.52 (m, 1H), 3.44-3.35 (m, 1H), 3.15-3.05 (m, 3H), 3.02-2.88 (m, 4H), 2.63-2.56 (m, 1H), 2.47 (s, 3H), 2.28-2.21 (m, 1H), 2.10-2.01 (m, 2H), 1.04 (s, 9H). HRMS m/z [M+H]⁺ calcd for C₅₁H₆₁N₈O₁₁S⁺ 993.4175, found 993.4178.

### Example 230

### Synthesis of LQ126-91

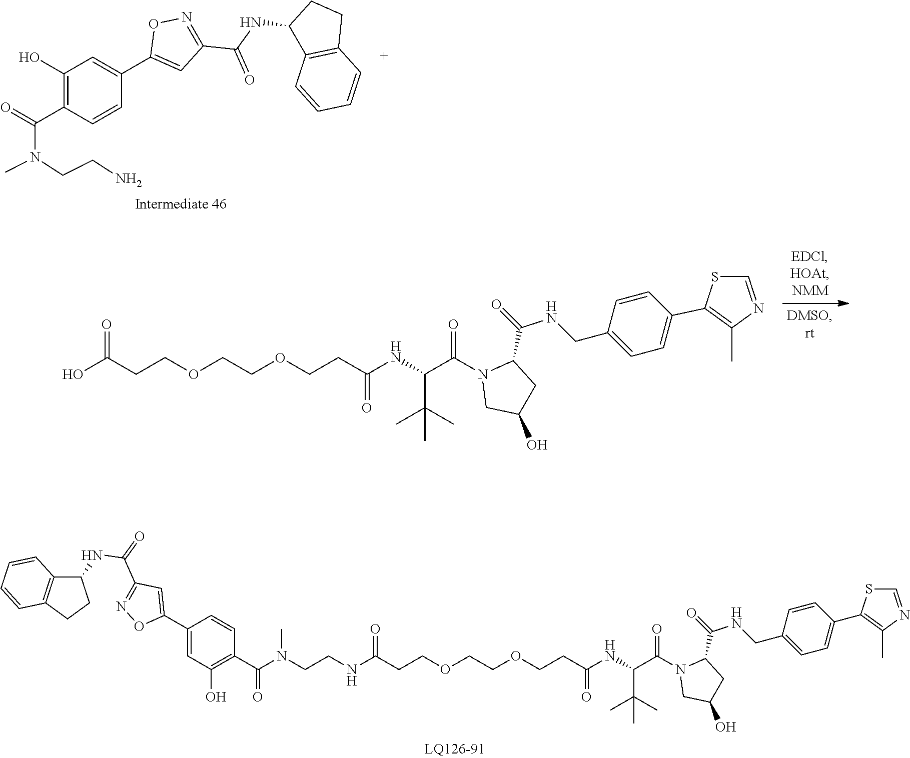 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)CCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCC(=O)NCCN(C)C(=O)c2ccc(-c3cc(C(=O)N[C@@H]4CCc5ccccc54)no3)cc2O)C(C)(C)C)cc1]

LQ126-91 was synthesized following the standard procedure for preparing LQ126-89 from intermediate 46 (4 mg, 0.01 mmol), 3-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)propanoic acid (6.2 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-91 was obtained as white solid (7.8 mg, 77%). ¹H NMR (600 MHz, Methanol-d₄) δ 9.00 (s, 1H), 7.49-7.40 (m, 5H), 7.39-7.35 (m, 2H), 7.34-7.20 (m, 4H), 7.15 (s, 1H), 5.65 (t, J=7.8 Hz, 1H), 4.67 (s, 1H), 4.61 (t, J=8.3 Hz, 1H), 4.54 (d, J=15.4 Hz, 1H), 4.52-4.49 (m, 1H), 4.36 (d, J=15.5 Hz, 1H), 3.91 (d, J=11.0 Hz, 1H), 3.81 (dd, J=11.0, 3.9 Hz, 1H), 3.78-3.48 (m, 10H), 3.44-3.36 (m, 2H), 3.17-3.07 (m, 3H), 3.03-2.91 (m, 4H), 2.64-2.52 (m, 2H), 2.49 (s, 3H), 2.42-2.33 (m, 1H), 2.27-2.21 (m, 1H), 2.13-2.04 (m, 2H), 1.05 (s, 9H). HRMS m/z [M+H]⁺ calcd for C₅₃H₆₅N₈O₁₁S⁺ 1021.4488, found 1021.4485.

### Example 231

### Synthesis of LQ126-92

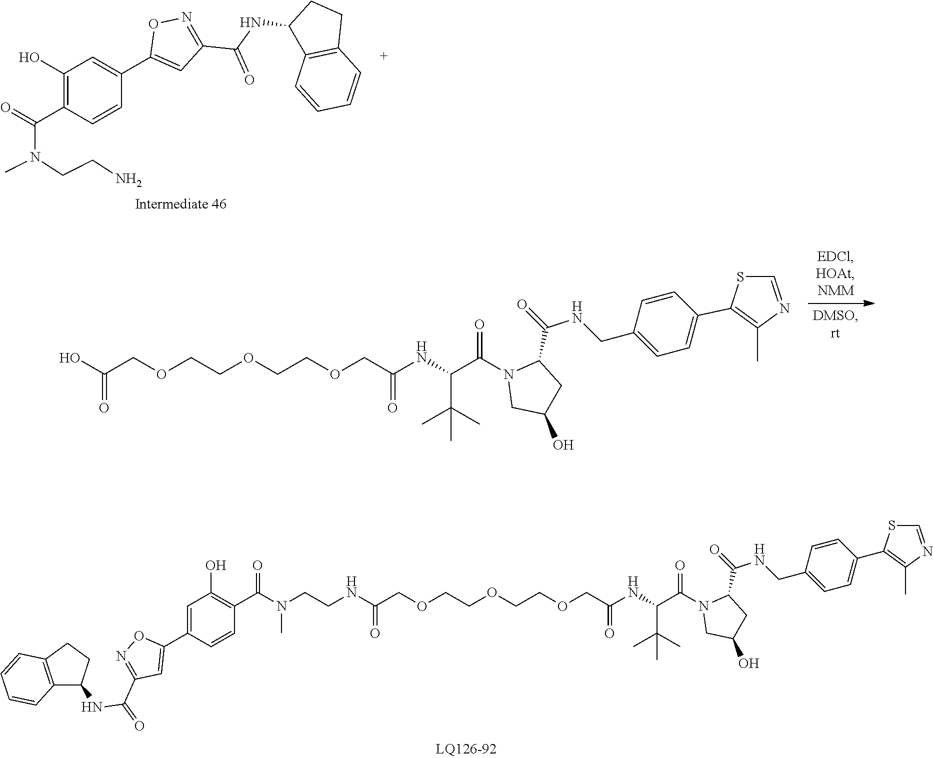 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)COCCOCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCCOCC(=O)NCCN(C)C(=O)c2ccc(-c3cc(C(=O)N[C@@H]4CCc5ccccc54)no3)cc2O)C(C)(C)C)cc1]

LQ126-92 was synthesized following the standard procedure for preparing LQ126-89 from intermediate 46 (4 mg, 0.01 mmol), (S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecanoic acid (6.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-92 was obtained as white solid (6.9 mg, 67%). ¹H NMR (600 MHz, Methanol-d₄) δ 8.97 (s, 1H), 7.49-7.41 (m, 5H), 7.39-7.31 (m, 3H), 7.29 (d, J=7.4 Hz, 1H), 7.27-7.20 (m, 2H), 7.15 (s, 1H), 5.65 (t, J=7.7 Hz, 1H), 4.74-4.70 (m, 1H), 4.62-4.48 (m, 3H), 4.35 (d, J=15.4 Hz, 1H), 4.11-3.97 (m, 4H), 3.87 (d, J=11.0 Hz, 1H), 3.81 (dd, J=11.0, 3.8 Hz, 1H), 3.78-3.64 (m, 8H), 3.48-3.38 (m, 2H), 3.18-3.07 (m, 3H), 3.02-2.91 (m, 4H), 2.65-2.57 (m, 1H), 2.49 (s, 3H), 2.24 (dd, J=13.2, 7.7 Hz, 1H), 2.13-2.04 (m, 2H), 1.05 (s, 9H). HRMS m/z [M+H]⁺ calcd for C₅₃H₆₅N₈O₁₂S⁺ 1037.4437, found 1037.4430.

### Example 232

### Synthesis of LQ126-93

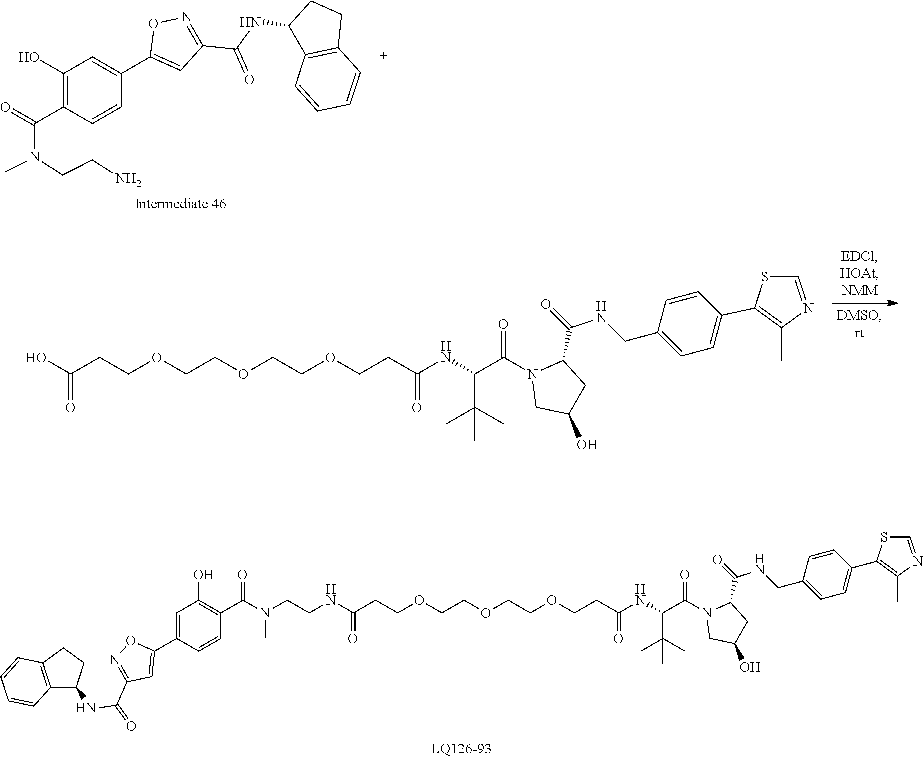 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)CCOCCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCOCCC(=O)NCCN(C)C(=O)c2ccc(-c3cc(C(=O)N[C@@H]4CCc5ccccc54)no3)cc2O)C(C)(C)C)cc1]

LQ126-93 was synthesized following the standard procedure for preparing LQ126-89 from intermediate 46 (4 mg, 0.01 mmol), (S)-15-((2S,4R)-4-hydroxy-2-((4-(4-methylthiazol-5-yl)benzyl)carbamoyl)pyrrolidine-1-carbonyl)-16,16-dimethyl-13-oxo-4,7,10-trioxa-14-azaheptadecanoic acid (6.6 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-93 was obtained as white solid (6.7 mg, 63%). ¹H NMR (600 MHz, Methanol-d₄) δ 9.06 (s, 1H), 7.50-7.47 (m, 2H), 7.45-7.41 (m, 3H), 7.39-7.35 (m, 2H), 7.32 (d, J=7.3 Hz, 1H), 7.29 (d, J=7.3 Hz, 1H), 7.27-7.20 (m, 2H), 7.16 (s, 1H), 5.66 (t, J=7.8 Hz, 1H), 4.66 (s, 1H), 4.60 (t, J=8.3 Hz, 1H), 4.55 (d, J=15.5 Hz, 1H), 4.52-4.49 (m, 1H), 4.37 (d, J=15.5 Hz, 1H), 3.91 (d, J=10.9 Hz, 1H), 3.81 (dd, J=11.0, 3.9 Hz, 1H), 3.78-3.66 (m, 6H), 3.64-3.50 (m, 10H), 3.18-3.06 (m, 3H), 3.04-2.91 (m, 4H), 2.64-2.54 (m, 2H), 2.50 (s, 3H), 2.49-2.45 (m, 2H), 2.44-2.37 (m, 1H), 2.26-2.21 (m, 1H), 2.12-2.04 (m, 2H), 1.05 (s, 9H). HRMS m/z [M+H]⁺ calcd for C₅₅H₆₉N₈O₁₂S⁺ 1065.4750, found 1065.4745.

### Example 233

### Synthesis of LQ126-94

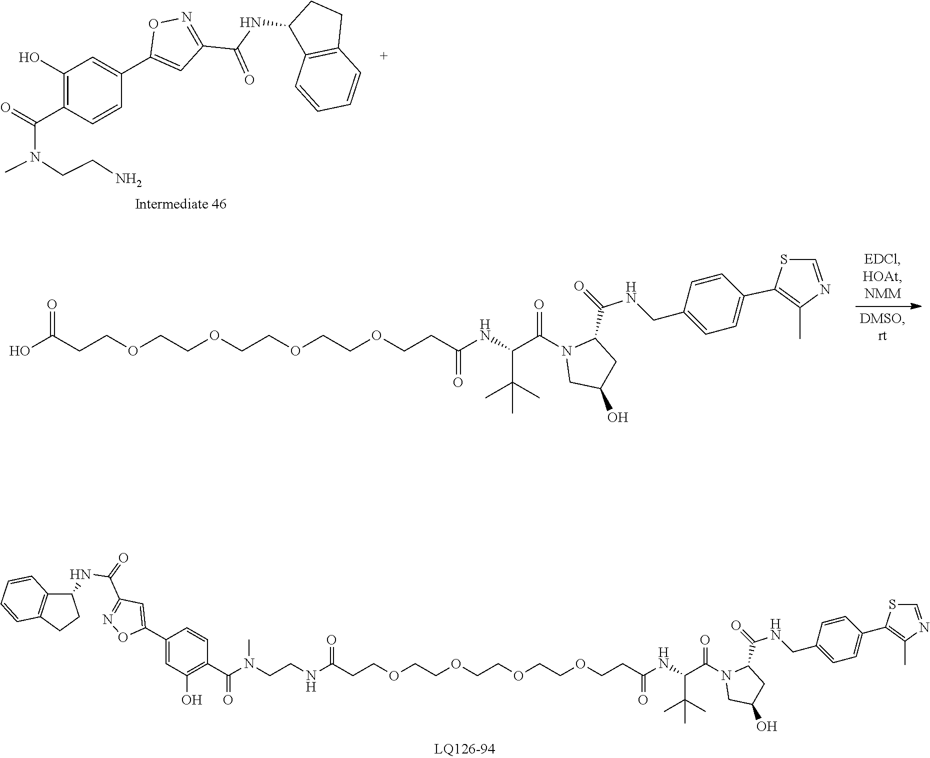 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)CCOCCOCCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCOCCOCCC(=O)NCCN(C)C(=O)c2ccc(-c3cc(C(=O)N[C@@H]4CCc5ccccc54)no3)cc2O)C(C)(C)C)cc1]

LQ126-94 was synthesized following the standard procedure for preparing LQ126-89 from intermediate 46 (4 mg, 0.01 mmol), (S)-18-((2S,4R)-4-hydroxy-2-((4-(4-methylthiazol-5-yl)benzyl)carbamoyl)pyrrolidine-1-carbonyl)-19,19-dimethyl-16-oxo-4,7,10,13-tetraoxa-17-azaicosanoic acid (7.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-94 was obtained as white solid (7.3 mg, 66%). ¹H NMR (600 MHz, Methanol-d₄) δ 9.05 (s, 1H), 7.49 (d, J=8.0 Hz, 2H), 7.46-7.41 (m, 3H), 7.39-7.36 (m, 2H), 7.32 (d, J=7.3 Hz, 1H), 7.29 (d, J=7.3 Hz, 1H), 7.27-7.20 (m, 2H), 7.16 (s, 1H), 5.66 (t, J=7.8 Hz, 1H), 4.66 (s, 1H), 4.62-4.57 (m, 1H), 4.55 (d, J=15.5 Hz, 1H), 4.52-4.49 (m, 1H), 4.37 (d, J=15.5 Hz, 1H), 3.91 (d, J=11.0 Hz, 1H), 3.81 (dd, J=10.9, 3.9 Hz, 1H), 3.78-3.50 (m, 18H), 3.18-3.07 (m, 3H), 3.04-2.91 (m, 4H), 2.65-2.54 (m, 2H), 2.50 (s, 3H), 2.49-2.45 (m, 2H), 2.43-2.34 (m, 1H), 2.27-2.20 (m, 1H), 2.13-2.03 (m, 2H), 1.05 (s, 9H). HRMS m/z [M+H]⁺ calcd for C₅₇H₇₃N₈O₁₃S⁺ 1109.5012, found 1109.5016.

### Example 234

### Synthesis of LQ126-95

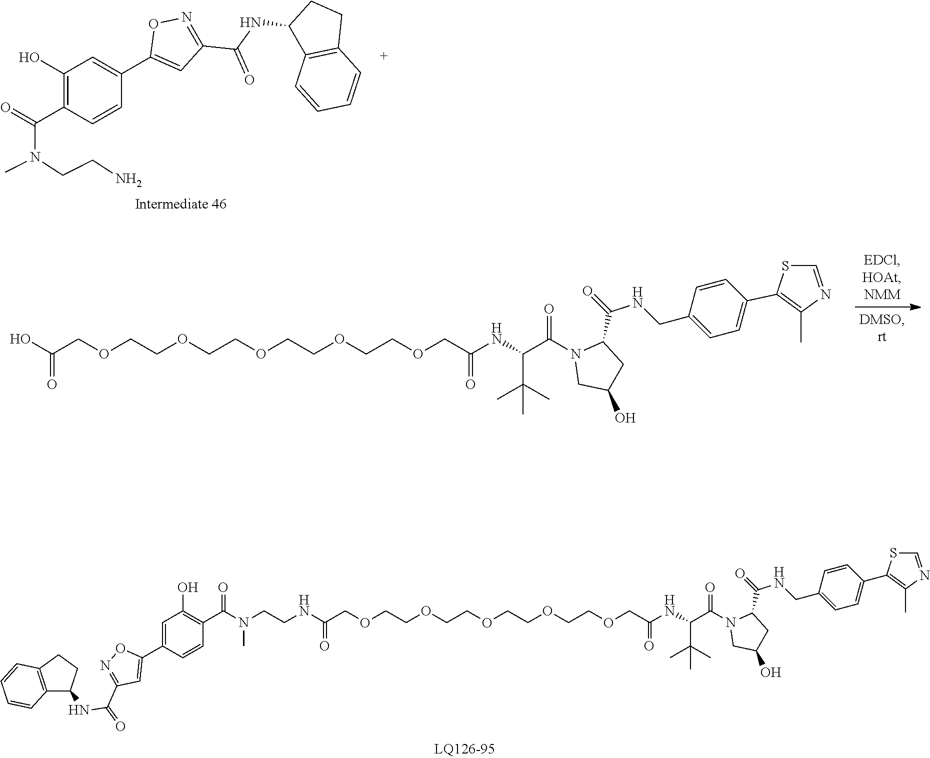 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)COCCOCCOCCOCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCCOCCOCCOCC(=O)NCCN(C)C(=O)c2ccc(-c3cc(C(=O)N[C@@H]4CCc5ccccc54)no3)cc2O)C(C)(C)C)cc1]

LQ126-95 was synthesized following the standard procedure for preparing LQ126-89 from intermediate 46 (4 mg, 0.01 mmol), (S)-19-((2S,4R)-4-hydroxy-2-((4-(4-methylthiazol-5-yl)benzyl)carbamoyl)pyrrolidine-1-carbonyl)-20,20-dimethyl-17-oxo-3,6,9,12,15-pentaoxa-18-azahenicosanoic acid (7.2 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-95 was obtained as white solid (7 mg, 62%). ¹H NMR (600 MHz, Methanol-d₄) δ 9.03 (s, 1H), 7.50-7.45 (m, 2H), 7.46-7.42 (m, 3H), 7.39-7.34 (m, 2H), 7.32 (d, J=7.3 Hz, 1H), 7.29 (d, J=7.4 Hz, 1H), 7.27-7.20 (m, 2H), 7.16 (s, 1H), 5.65 (t, J=7.7 Hz, 1H), 4.73-4.69 (m, 1H), 4.63-4.50 (m, 3H), 4.37 (d, J=15.5 Hz, 1H), 4.08-4.00 (m, 4H), 3.96-3.87 (m, 2H), 3.81 (dd, J=11.0, 3.8 Hz, 1H), 3.78-3.54 (m, 16H), 3.47-3.37 (m, 1H), 3.19-3.06 (m, 3H), 3.05-2.91 (m, 4H), 2.65-2.57 (m, 1H), 2.50 (s, 3H), 2.27-2.22 (m, 1H), 2.13-2.04 (m, 2H), 1.05 (s, 9H). HRMS m/z [M+H]⁺ calcd for C₅₇H₇₃N₈O₁₃S⁺ 1125.4961, found 1125.4967.

### Example 235

### Synthesis of LQ126-96

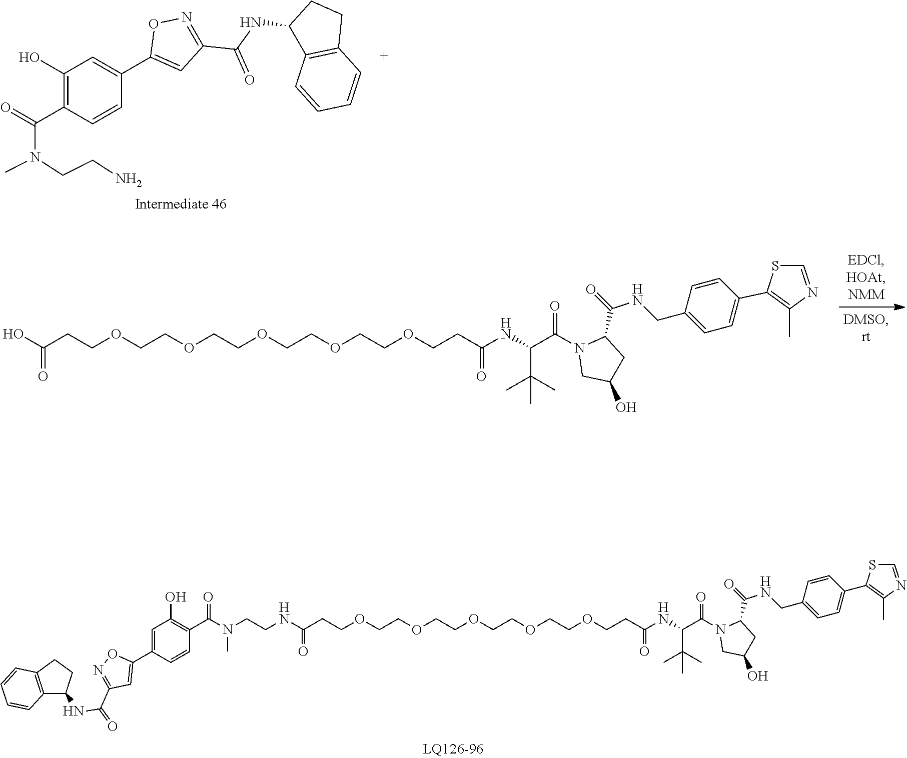 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)CCOCCOCCOCCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCOCCOCCOCCC(=O)NCCN(C)C(=O)c2ccc(-c3cc(C(=O)N[C@@H]4CCc5ccccc54)no3)cc2O)C(C)(C)C)cc1]

LQ126-96 was synthesized following the standard procedure for preparing LQ126-89 from intermediate 46 (4 mg, 0.01 mmol), (S)-21-((2S,4R)-4-hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azatricosanoic acid (7.5 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-96 was obtained as white solid (6.5 mg, 56%). ¹H NMR (600 MHz, Methanol-d₄) δ 9.07 (s, 1H), 7.49 (d, J=8.0 Hz, 2H), 7.46-7.42 (m, 3H), 7.39-7.36 (m, 2H), 7.32 (d, J=7.3 Hz, 1H), 7.29 (d, J=7.4 Hz, 1H), 7.27-7.20 (m, 2H), 7.16 (s, 1H), 5.66 (t, J=7.7 Hz, 1H), 4.69-4.65 (m, 1H), 4.61-4.58 (m, 1H), 4.56 (d, J=15.5 Hz, 1H), 4.52-4.49 (m, 1H), 4.37 (d, J=15.5 Hz, 1H), 3.90 (d, J=11.0 Hz, 1H), 3.81 (dd, J=11.0, 3.9 Hz, 1H), 3.78-3.51 (m, 22H), 3.18-3.07 (m, 3H), 3.03-2.91 (m, 4H), 2.64-2.55 (m, 2H), 2.51 (s, 3H), 2.50-2.46 (m, 2H), 2.43-2.37 (m, 1H), 2.26-2.21 (m, 1H), 2.12-2.04 (m, 2H), 1.05 (s, 9H). HRMS m/z [M+H]⁺ calcd for C₅₉H₇₇N₈O₁₄S⁺ 1153.5274, found 1153.5270.

### Example 236

### Synthesis of LQ126-97

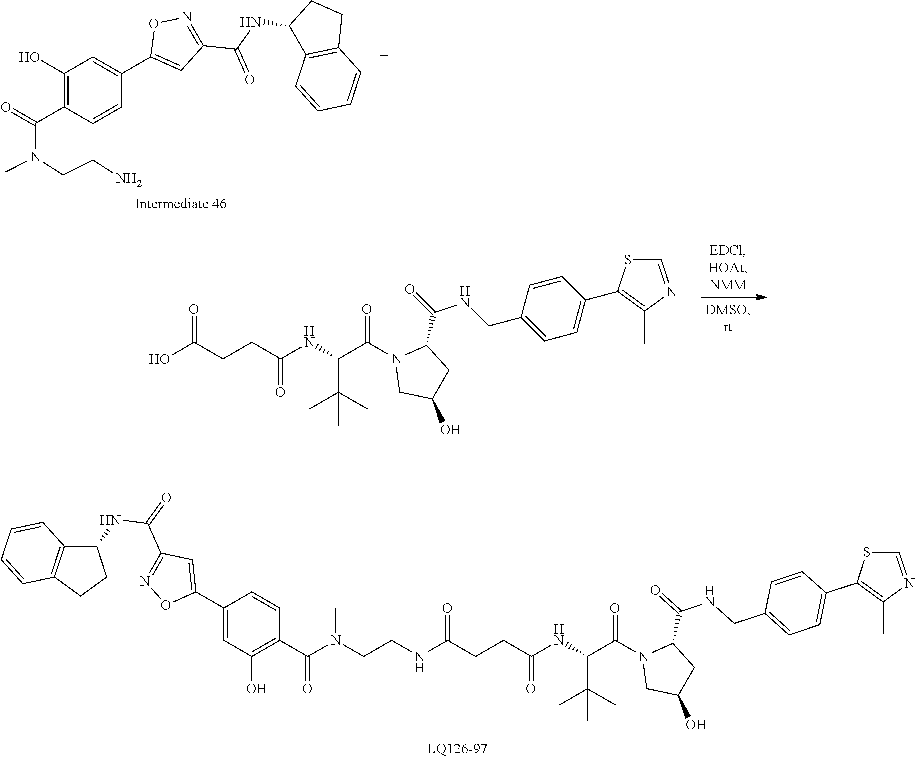 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)CCC(=O)O)C(C)(C)C)cc1 ; Cc1ncsc1-c1ccc(CNC(=O)[C@@H]2C[C@@H](O)CN2C(=O)[C@@H](NC(=O)CCC(=O)NCCN(C)C(=O)c2ccc(-c3cc(C(=O)N[C@@H]4CCc5ccccc54)no3)cc2O)C(C)(C)C)cc1]

LQ126-97 was synthesized following the standard procedure for preparing LQ126-89 from intermediate 46 (4 mg, 0.01 mmol), 4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-4-oxobutanoic acid (5.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-97 was obtained as white solid (5.8 mg, 62%). ¹H NMR (600 MHz, Methanol-d₄) δ 8.99 (s, 1H), 7.49-7.20 (m, 11H), 7.18-7.10 (m, 1H), 5.68-5.61 (m, 1H), 4.64-4.49 (m, 4H), 4.37 (d, J=15.5 Hz, 1H), 3.93 (d, J=11.0 Hz, 1H), 3.84-3.79 (m, 1H), 3.77-3.54 (m, 1H), 3.51-3.37 (m, 1H), 3.18-3.06 (m, 3H), 3.03-2.90 (m, 4H), 2.65-2.41 (m, 8H), 2.28-2.21 (m, 1H), 2.12-2.03 (m, 2H), 1.05 (s, 9H). HRMS m/z [M+H]⁺ calcd for C₄₉H₅₇N₈O₉S⁺ 933.3964, found 933.3966.

### Example 237

### Synthesis of LQ126-98

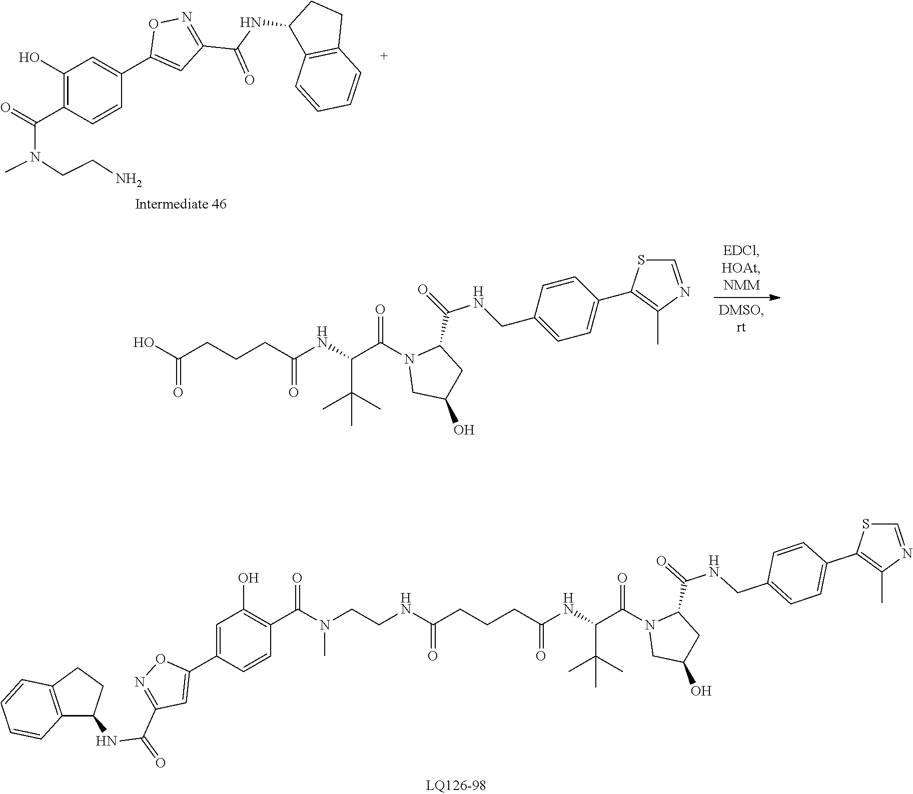 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)CCCC(=O)O)C(C)(C)C)cc1 ; Cc1ncsc1-c1ccc(CNC(=O)[C@@H]2C[C@@H](O)CN2C(=O)[C@@H](NC(=O)CCCC(=O)NCCN(C)C(=O)c2ccc(-c3cc(C(=O)N[C@@H]4CCc5ccccc54)no3)cc2O)C(C)(C)C)cc1]

LQ126-98 was synthesized following the standard procedure for preparing LQ126-89 from intermediate 46 (4 mg, 0.01 mmol), 5-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-5-oxopentanoic acid (5.4 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-98 was obtained as white solid (6.4 mg, 68%). ¹H NMR (600 MHz, Methanol-d₄) δ 9.05 (s, 1H), 7.49-7.40 (m, 6H), 7.38-7.31 (m, 2H), 7.31-7.20 (m, 3H), 7.15 (s, 1H), 5.65 (t, J=7.7 Hz, 1H), 4.66-4.60 (m, 2H), 4.56-4.51 (m, 2H), 4.36 (d, J=15.5 Hz, 1H), 3.97 (d, J=11.0 Hz, 1H), 3.83 (dd, J=10.9, 3.9 Hz, 1H), 3.79-3.39 (m, 2H), 3.18-3.06 (m, 3H), 3.05-2.90 (m, 4H), 2.65-2.58 (m, 1H), 2.49 (s, 3H), 2.39-2.06 (m, 7H), 2.00-1.79 (m, 2H), 1.06 (s, 9H). HRMS m/z [M+H]+ calcd for C₅₀H₅₉N₈O₉S⁺ 947.4120, found 947.4149.

### Example 238

### Synthesis of LQ126-99

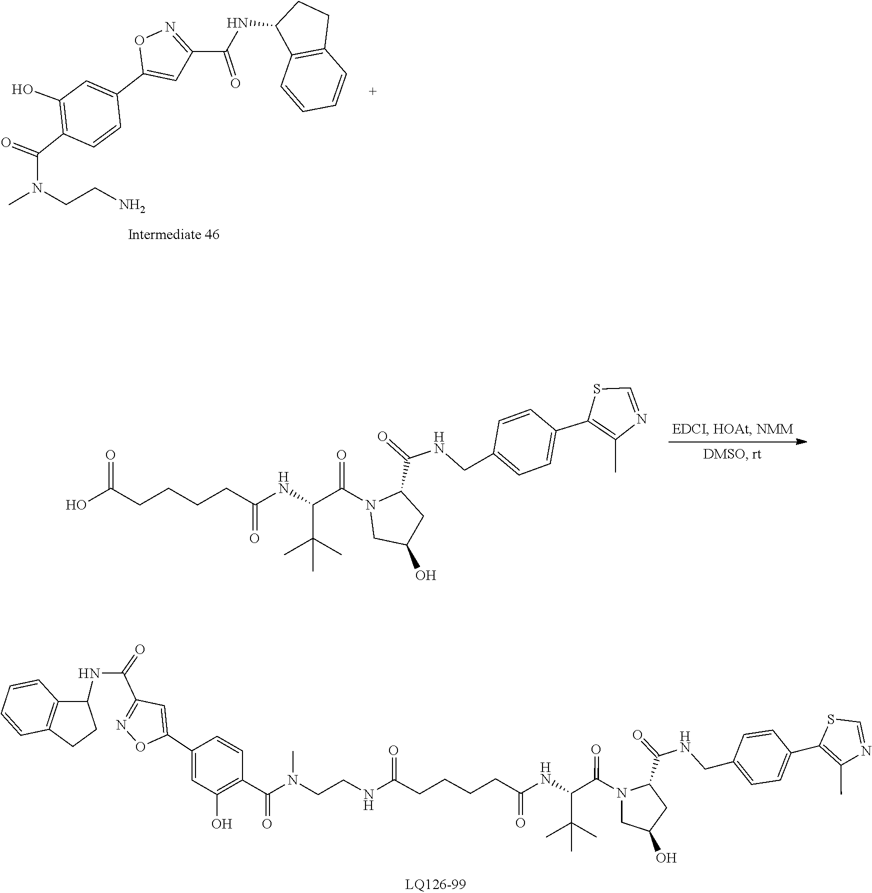 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)CCCCC(=O)O)C(C)(C)C)cc1 ; Cc1ncsc1-c1ccc(CNC(=O)[C@@H]2C[C@@H](O)CN2C(=O)[C@@H](NC(=O)CCCCC(=O)NCCN(C)C(=O)c2ccc(-c3cc(C(=O)NC4CCc5ccccc54)no3)cc2O)C(C)(C)C)cc1]

LQ126-99 was synthesized following the standard procedure for preparing LQ126-89 from intermediate 46 (4 mg, 0.01 mmol), 6-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-6-oxohexanoic acid (5.5 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-99 was obtained as white solid (6.7 mg, 70%). ¹H NMR (600 MHz, Methanol-d₄) δ 9.06 (s, 1H), 7.50-7.41 (m, 5H), 7.39-7.20 (m, 6H), 7.15 (s, 1H), 5.65 (t, J=7.8 Hz, 1H), 4.67-4.49 (m, 4H), 4.37 (d, J=15.5 Hz, 1H), 3.93 (d, J=11.1 Hz, 1H), 3.82 (dd, J=11.0, 3.9 Hz, 1H), 3.56-3.50 (m, 1H), 3.45-3.37 (m, 1H), 3.18-3.05 (m, 3H), 3.03-2.91 (m, 4H), 2.65-2.57 (m, 1H), 2.50 (s, 3H), 2.37-2.03 (m, 8H), 1.72-1.52 (m, 3H), 1.05 (s, 9H). HRMS m/z [M+H]⁺ calcd for C₅₁H₆₁N₈O₉S⁺ 961.4277, found 961.4277.

### Example 239

### Synthesis of LQ126-100

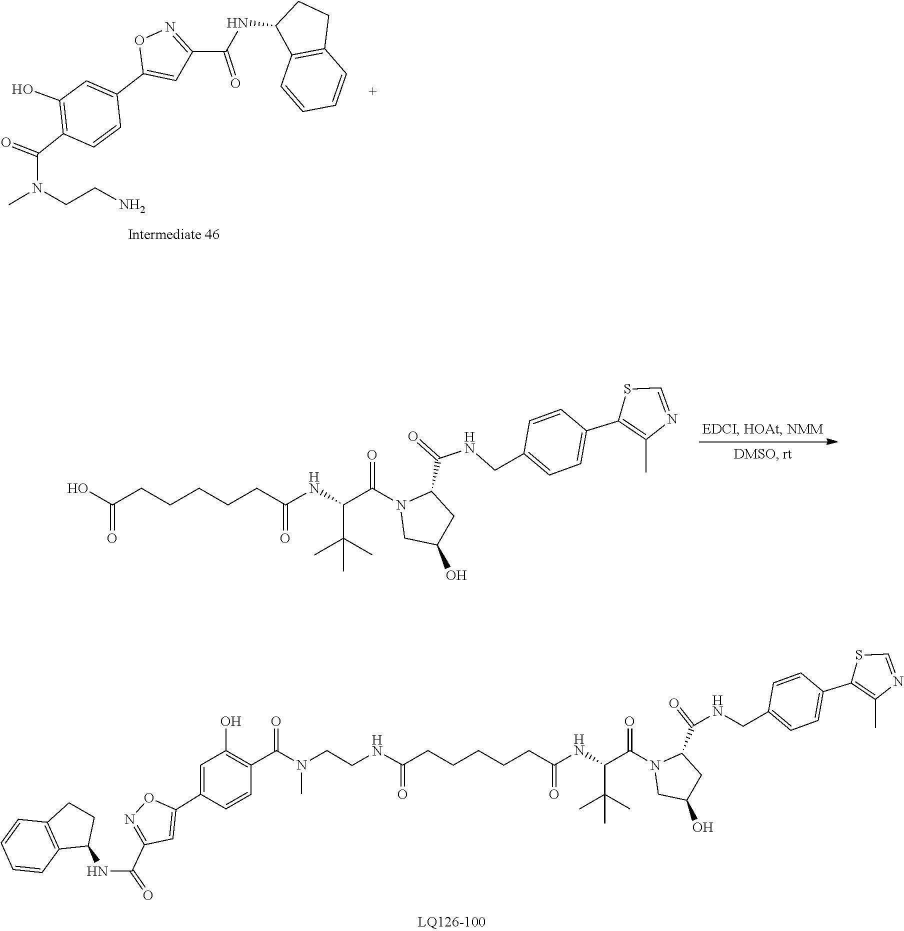 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)CCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCC(=O)NCCN(C)C(=O)c2ccc(-c3cc(C(=O)N[C@@H]4CCc5ccccc54)no3)cc2O)C(C)(C)C)cc1]

LQ126-100 was synthesized following the standard procedure for preparing LQ126-89 from intermediate 46 (4 mg, 0.01 mmol), 7-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-7-oxoheptanoic acid (5.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-100 was obtained as white solid (6.3 mg, 65%). ¹H NMR (600 MHz, Methanol-d₄) δ 8.92 (s, 1H), 7.49-7.39 (m, 6H), 7.40-7.31 (m, 2H), 7.30-7.20 (m, 3H), 7.16 (s, 1H), 5.65 (t, J=7.6 Hz, 1H), 4.65 (s, 1H), 4.60 (t, J=8.6 Hz, 1H), 4.57-4.49 (m, 2H), 4.36 (d, J=15.5 Hz, 1H), 3.92 (d, J=10.9 Hz, 1H), 3.82 (dd, J=10.9, 3.9 Hz, 1H), 3.74-3.68 (m, 1H), 3.56-3.49 (m, 1H), 3.18-3.06 (m, 3H), 3.03-2.91 (m, 4H), 2.65-2.57 (m, 1H), 2.48 (s, 3H), 2.32-2.19 (m, 4H), 2.17-2.04 (m, 2H), 1.71-1.50 (m, 4H), 1.43-1.28 (m, 3H), 1.05 (s, 9H). HRMS m/z [M+H]⁺ calcd for C₅₂H₆₃N₈O₉S⁺ 975.4433, found 975.4413.

### Example 240

### Synthesis of LQ126-101

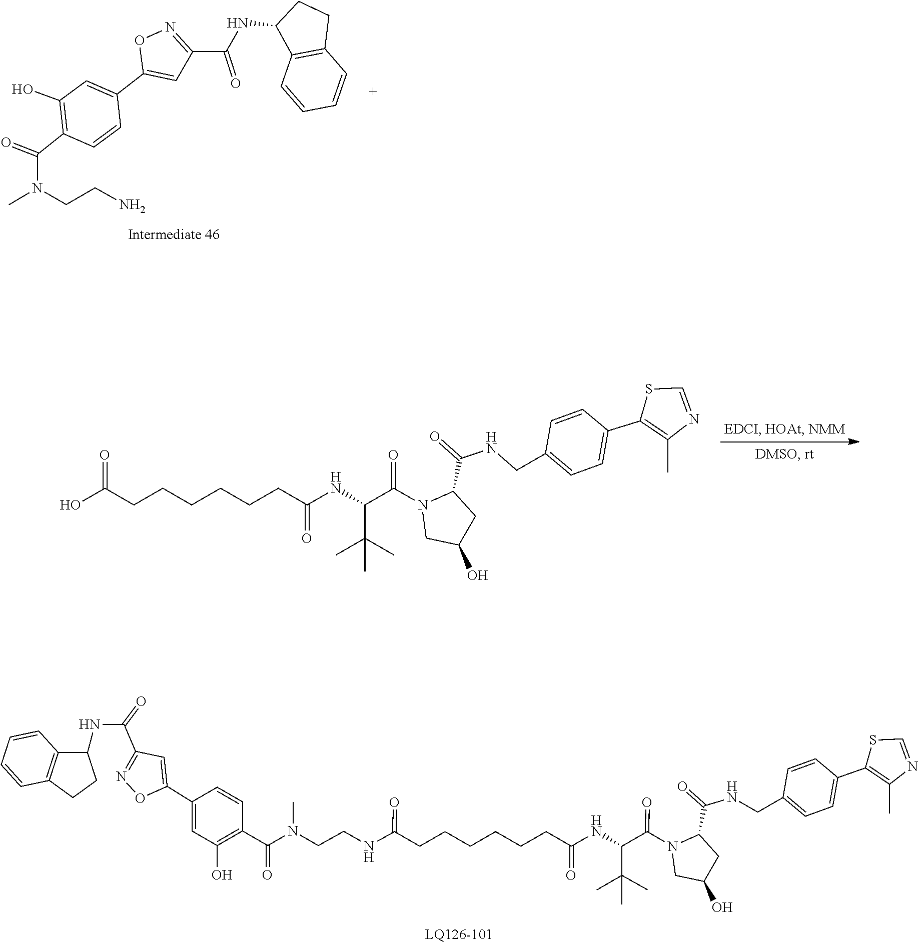 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)CCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCC(=O)NCCN(C)C(=O)c2ccc(-c3cc(C(=O)NC4CCc5ccccc54)no3)cc2O)C(C)(C)C)cc1]

LQ126-101 was synthesized following the standard procedure for preparing LQ126-89 from intermediate 46 (4 mg, 0.01 mmol), 8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctanoic acid (5.8 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-101 was obtained as white solid (7.2 mg, 73%). ¹H NMR (600 MHz, Methanol-d₄) δ 8.93 (s, 1H), 7.48-7.39 (m, 5H), 7.37-7.18 (m, 6H), 7.14 (s, 1H), 5.64 (t, J=7.8 Hz, 1H), 4.63 (s, 1H), 4.60-4.47 (m, 3H), 4.34 (d, J=15.4 Hz, 1H), 3.90 (d, J=11.1 Hz, 1H), 3.80 (dd, J=11.0, 3.9 Hz, 1H), 3.71-3.59 (m, 1H), 3.52-3.35 (m, 1H), 3.17-3.04 (m, 3H), 3.02-2.88 (m, 4H), 2.62-2.56 (m, 1H), 2.47 (s, 3H), 2.32-2.02 (m, 4H), 1.66-1.46 (m, 5H), 1.43-1.26 (m, 6H), 1.03 (s, 9H). HRMS m/z [M+H]⁺ calcd for C₅₃H₆₅N₈O₉S⁺ 989.4590, found 989.4602.

### Example 241

### Synthesis of LQ126-102

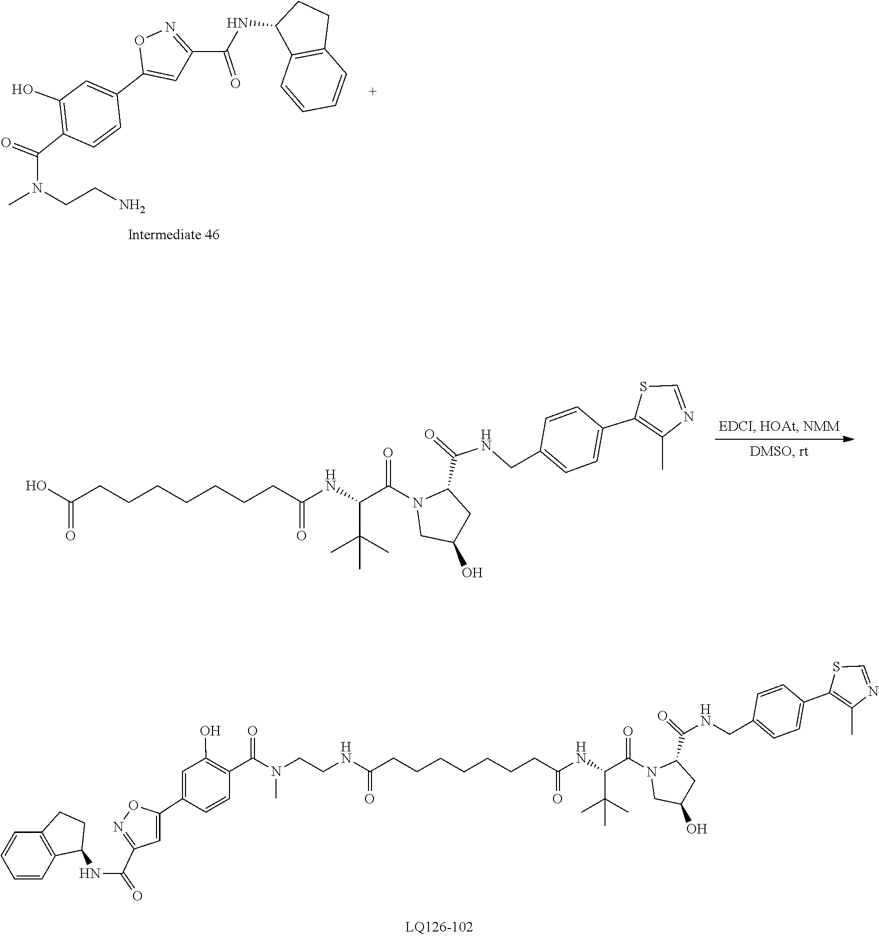 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)CCCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCCC(=O)NCCN(C)C(=O)c2ccc(-c3cc(C(=O)N[C@@H]4CCc5ccccc54)no3)cc2O)C(C)(C)C)cc1]

LQ126-102 was synthesized following the standard procedure for preparing LQ126-89 from intermediate 46 (4 mg, 0.01 mmol), 9-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-9-oxononanoic acid (6 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-102 was obtained as white solid (7.1 mg, 71%). ¹H NMR (600 MHz, Methanol-d₄) δ 8.98 (s, 1H), 7.50-7.46 (m, 2H), 7.46-7.41 (m, 3H), 7.39-7.31 (m, 3H), 7.30-7.20 (m, 3H), 7.16 (s, 1H), 5.66 (t, J=7.7 Hz, 1H), 4.65 (s, 1H), 4.62-4.48 (m, 3H), 4.37 (d, J=15.4 Hz, 1H), 3.92 (d, J=11.0 Hz, 1H), 3.81 (dd, J=10.9, 3.9 Hz, 1H), 3.74-3.67 (m, 1H), 3.54-3.48 (m, 1H), 3.16-3.06 (m, 3H), 3.03-2.90 (m, 4H), 2.64-2.57 (m, 1H), 2.49 (s, 3H), 2.34-2.20 (m, 3H), 2.16-2.03 (m, 2H), 1.68-1.50 (m, 4H), 1.40-1.24 (m, 8H), 1.05 (s, 9H). HRMS m/z [M+H]⁺ calcd for C₅₄H₆₇N₈O₉S⁺ 1003.4746, found 1003.4739.

### Example 242

### Synthesis of LQ126-103

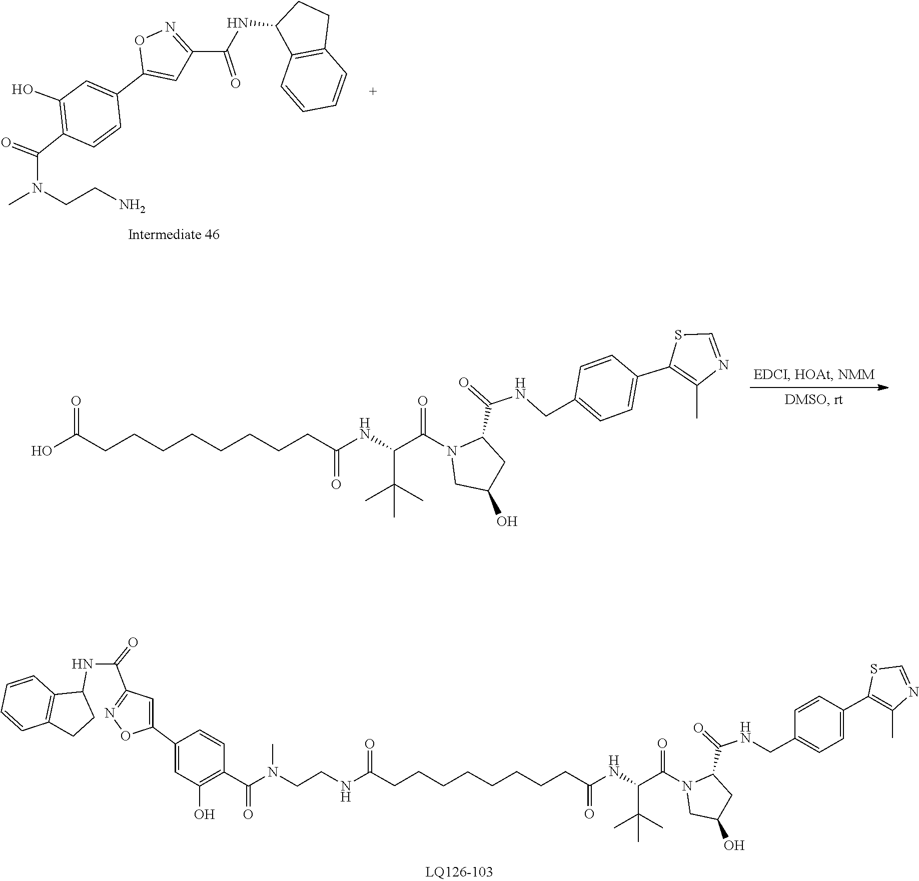 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)CCCCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCCCC(=O)NCCN(C)C(=O)c2ccc(-c3cc(C(=O)NC4CCc5ccccc54)no3)cc2O)C(C)(C)C)cc1]

LQ126-103 was synthesized following the standard procedure for preparing LQ126-89 from intermediate 46 (4 mg, 0.01 mmol), 10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecanoic acid (6.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-103 was obtained as white solid (6.5 mg, 64%). ¹H NMR (600 MHz, Methanol-d₄) δ 9.06 (s, 1H), 7.51-7.42 (m, 5H), 7.39-7.31 (m, 3H), 7.30-7.20 (m, 3H), 7.16 (s, 1H), 5.66 (t, J=7.8 Hz, 1H), 4.65 (s, 1H), 4.61-4.48 (m, 3H), 4.37 (d, J=15.5 Hz, 1H), 3.92 (d, J=11.0 Hz, 1H), 3.81 (dd, J=10.9, 3.9 Hz, 1H), 3.74-3.64 (m, 1H), 3.56-3.49 (m, 1H), 3.17-3.07 (m, 3H), 3.04-2.90 (m, 4H), 2.65-2.57 (m, 1H), 2.50 (s, 3H), 2.33-2.20 (m, 4H), 2.16-2.05 (m, 2H), 1.68-1.48 (m, 4H), 1.40-1.26 (m, 9H), 1.05 (s, 9H). HRMS m/z [M+H]⁺ calcd for C₅₅H₆₉N₈O₉S⁺ 1017.4903, found 1017.4902.

### Example 243

### Synthesis of LQ126-104

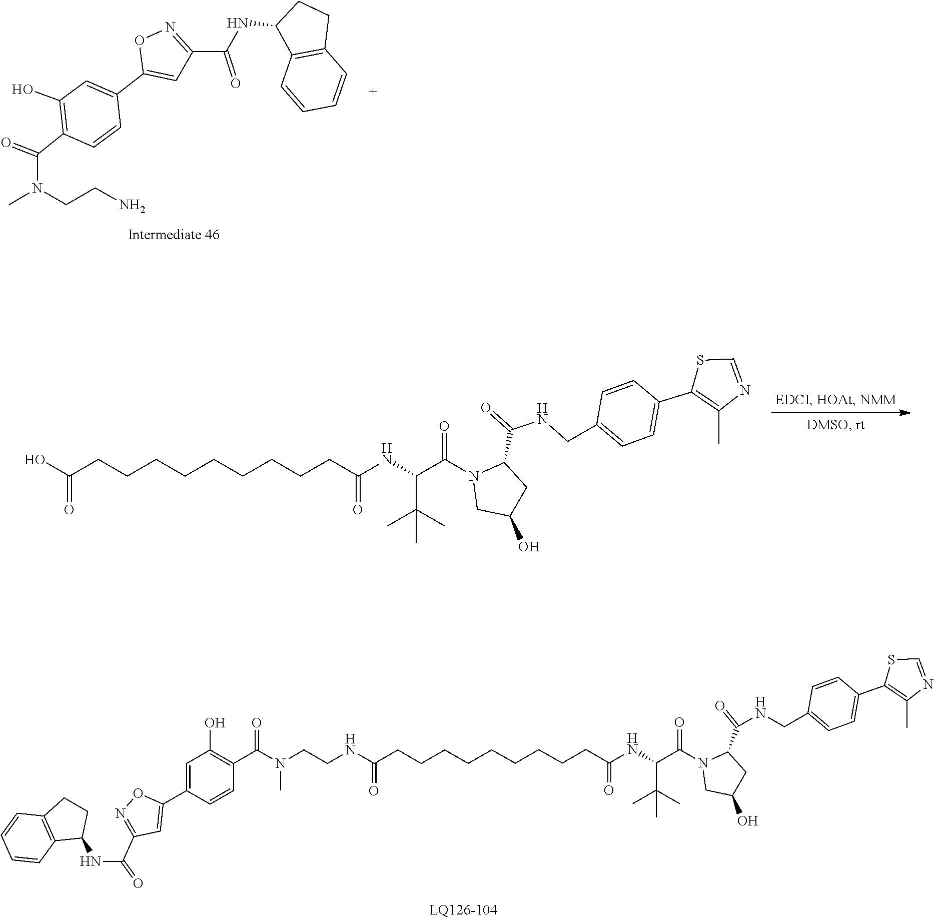 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)CCCCCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCCCCC(=O)NCCN(C)C(=O)c2ccc(-c3cc(C(=O)N[C@@H]4CCc5ccccc54)no3)cc2O)C(C)(C)C)cc1]

LQ126-104 was synthesized following the standard procedure for preparing LQ126-89 from intermediate 46 (4 mg, 0.01 mmol), 11-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-11-oxoundecanoic acid (6.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-104 was obtained as white solid (7 mg, 68%). ¹H NMR (600 MHz, Methanol-d₄) δ 8.97 (s, 1H), 7.50-7.42 (m, 5H), 7.40-7.31 (m, 3H), 7.30-7.20 (m, 3H), 7.16 (s, 1H), 5.66 (t, J=7.7 Hz, 1H), 4.65 (s, 1H), 4.62-4.48 (m, 3H), 4.37 (d, J=15.5 Hz, 1H), 3.92 (d, J=10.9 Hz, 1H), 3.82 (dd, J=11.0, 4.0 Hz, 1H), 3.73-3.68 (m, 1H), 3.56-3.50 (m, 1H), 3.17-3.06 (m, 3H), 3.04-2.90 (m, 4H), 2.65-2.57 (m, 1H), 2.49 (s, 3H), 2.33-2.19 (m, 4H), 2.14-2.03 (m, 2H), 1.68-1.50 (m, 5H), 1.39-1.25 (m, 10H), 1.05 (s, 9H). HRMS m/z [M+H]⁺ calcd for C₅₆H₇₁NO₉S⁺ 1031.5059, found 1031.5083.

### Example 244

### Synthesis of LQ126-105

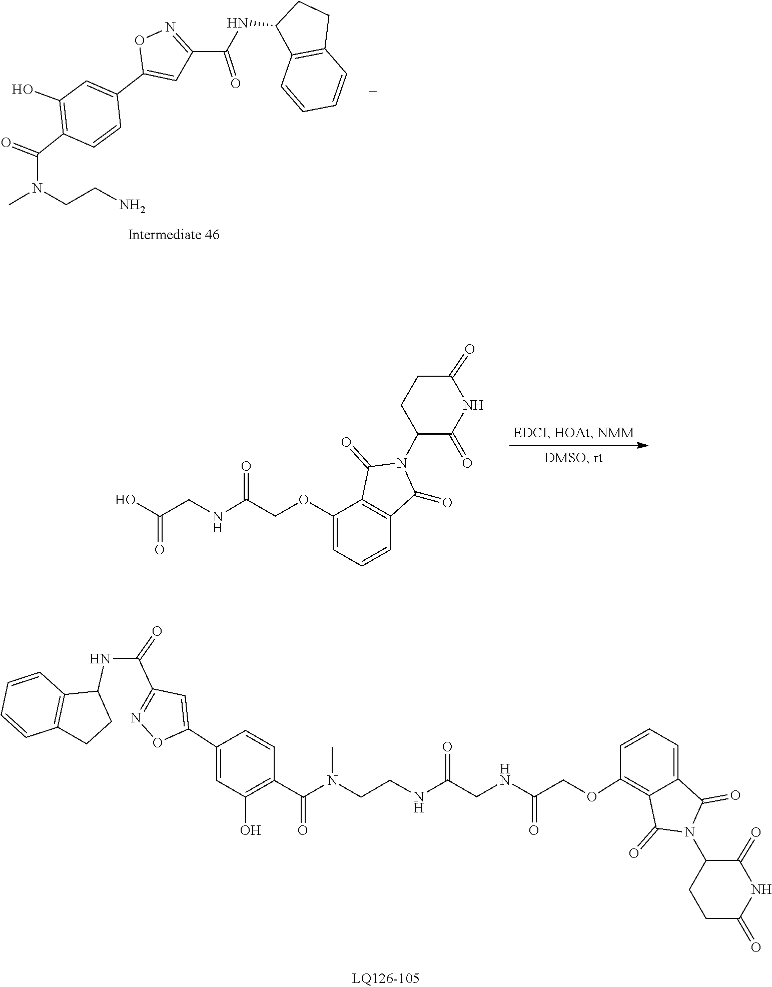 [SMILES: CN(CCN)C(=O)c1ccc(-c2cc(C(=O)N[C@@H]3CCc4ccccc43)no2)cc1O ; O=C(O)CNC(=O)COc1cccc2c1C(=O)N(C1CCC(=O)NC1=O)C2=O ; CN(CCNC(=O)CNC(=O)COc1cccc2c1C(=O)N(C1CCC(=O)NC1=O)C2=O)C(=O)c1ccc(-c2cc(C(=O)NC3CCc4ccccc43)no2)cc1O]

LQ126-105 was synthesized following the standard procedure for preparing LQ126-89 from intermediate 46 (4 mg, 0.01 mmol), (2-((2-(2,6-dioxopiperidin-3-yl)-1,3-dioxoisoindolin-4-yl)oxy)acetyl)glycine (3.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-105 was obtained as white solid (4.7 mg, 59%). ¹H NMR (600 MHz, Methanol-d₄) δ 7.79-7.72 (m, 1H), 7.53-7.18 (m, 9H), 7.12-7.03 (m, 1H), 5.68-5.58 (m, 1H), 5.12 (dd, J=12.8, 5.4 Hz, 1H), 4.03-3.87 (m, 2H), 3.75-3.66 (m, 1H), 3.58-3.43 (m, 2H), 3.40-3.33 (m, 2H), 3.15-3.04 (m, 3H), 3.01-2.80 (m, 4H), 2.78-2.67 (m, 2H), 2.63-2.56 (m, 1H), 2.17-2.02 (m, 2H). HRMS m/z [M+H]⁺ calcd for C₄₀H₃₈N₇O₁₁⁺ 792.2624, found 792.2635.

### Example 245

### Synthesis of LQ126-106

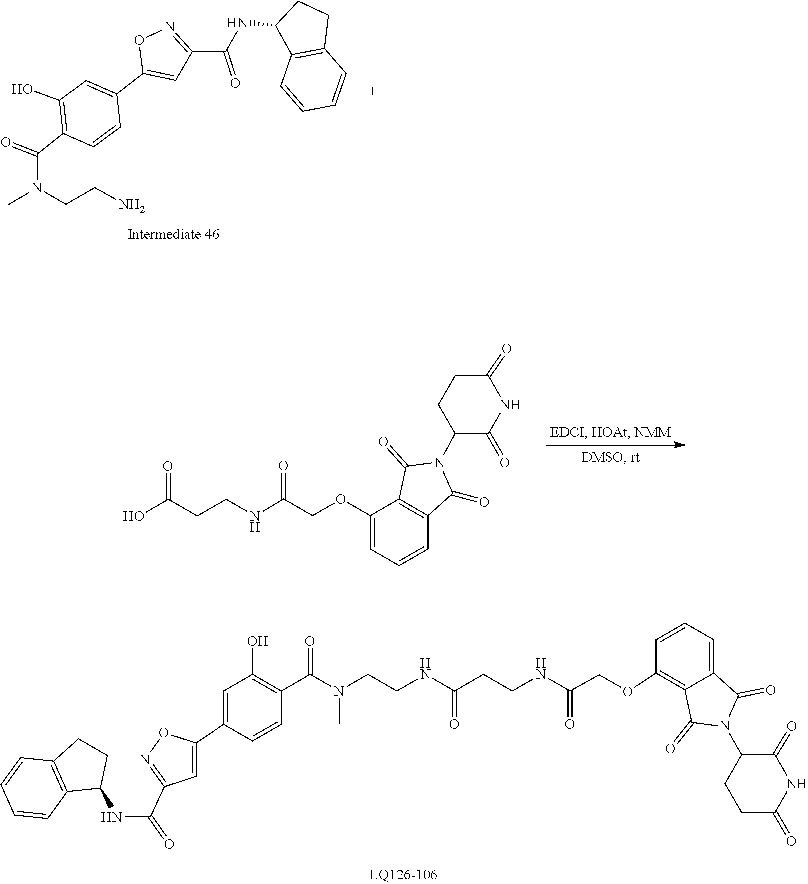 [SMILES: CN(CCN)C(=O)c1ccc(-c2cc(C(=O)N[C@@H]3CCc4ccccc43)no2)cc1O ; O=C(O)CCNC(=O)COc1cccc2c1C(=O)N(C1CCC(=O)NC1=O)C2=O ; CN(CCNC(=O)CCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O]

LQ126-106 was synthesized following the standard procedure for preparing LQ126-89 from intermediate 46 (4 mg, 0.01 mmol), 3-(2-((2-(2,6-dioxopiperidin-3-yl)-1,3-dioxoisoindolin-4-yl)oxy)acetamido)propanoic acid (4 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-106 was obtained as white solid (5.1 mg, 63%). ¹H NMR (600 MHz, Methanol-d₄) δ 7.83-7.74 (m, 1H), 7.56-7.46 (m, 1H), 7.44-7.21 (m, 8H), 7.12 (s, 1H), 5.66 (t, J=7.7 Hz, 1H), 5.14 (ddd, J=12.5, 5.6, 3.7 Hz, 1H), 4.74 (s, 2H), 3.76-3.36 (m, 7H), 3.16-3.06 (m, 2H), 3.03-2.83 (m, 3H), 2.80-2.71 (m, 2H), 2.66-2.58 (m, 1H), 2.55-2.39 (m, 2H), 2.18-2.13 (m, 1H), 2.13-2.04 (m, 1H). HRMS m/z [M+H]⁺ calcd for C₄₁H₄₀N₇O₁₁⁺ 806.2780, found 806.2771.

### Example 246

### Synthesis of LQ126-107

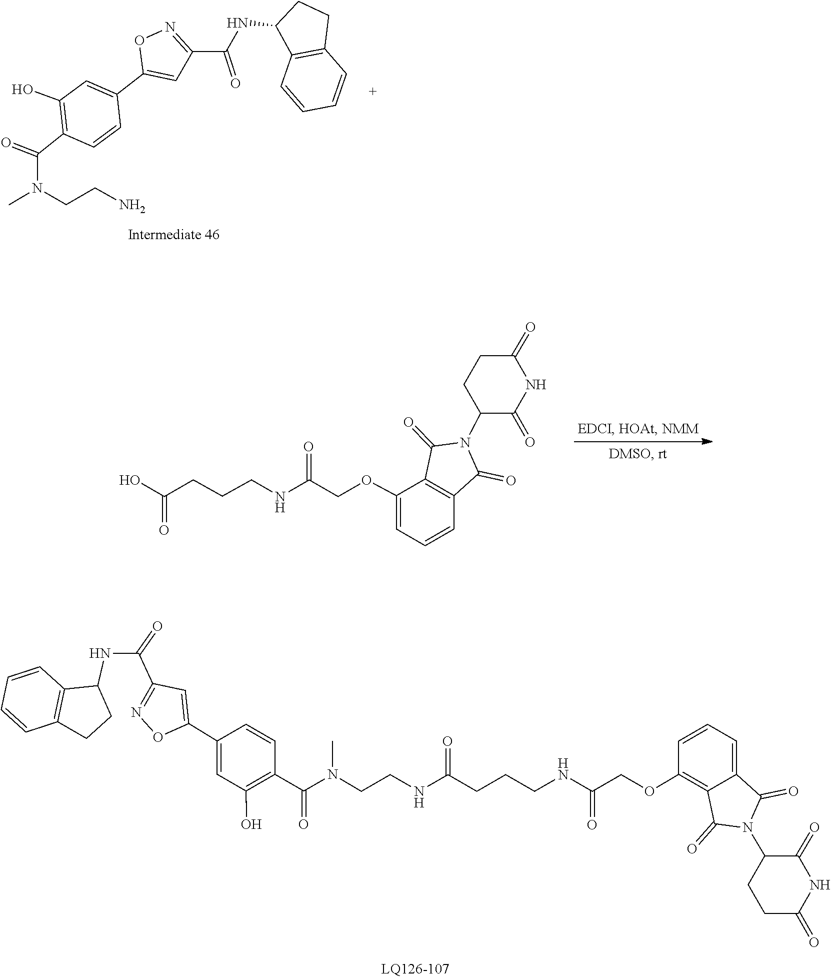 [SMILES: CN(CCN)C(=O)c1ccc(-c2cc(C(=O)N[C@@H]3CCc4ccccc43)no2)cc1O ; O=C(O)CCCNC(=O)COc1cccc2c1C(=O)N(C1CCC(=O)NC1=O)C2=O ; CN(CCNC(=O)CCCNC(=O)COc1cccc2c1C(=O)N(C1CCC(=O)NC1=O)C2=O)C(=O)c1ccc(-c2cc(C(=O)NC3CCc4ccccc43)no2)cc1O]

LQ126-107 was synthesized following the standard procedure for preparing LQ126-89 from intermediate 46 (4 mg, 0.01 mmol), 4-(2-((2-(2,6-dioxopiperidin-3-yl)-1,3-dioxoisoindolin-4-yl)oxy)acetamido)butanoic acid (4.2 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-107 was obtained as white solid (5.1 mg, 62%). ¹H NMR (600 MHz, Methanol-d₄) δ 7.80 (t, J=7.9 Hz, 1H), 7.55-7.51 (m, 1H), 7.47-7.39 (m, 2H), 7.36-7.20 (m, 6H), 7.13 (s, 1H), 5.68-5.62 (m, 1H), 5.17-5.10 (m, 1H), 4.78 (s, 2H), 3.76-3.63 (m, 1H), 3.57-3.50 (m, 1H), 3.45-3.36 (m, 5H), 3.16-3.07 (m, 2H), 3.05-2.83 (m, 3H), 2.80-2.69 (m, 2H), 2.64-2.56 (m, 1H), 2.35-2.03 (m, 4H), 1.94-1.75 (m, 2H). HRMS m/z [M+H]⁺ calcd for C₄₂H₄₂N₇O₁₁⁺ 820.2937, found 820.2938.

### Example 247

### Synthesis of LQ126-108

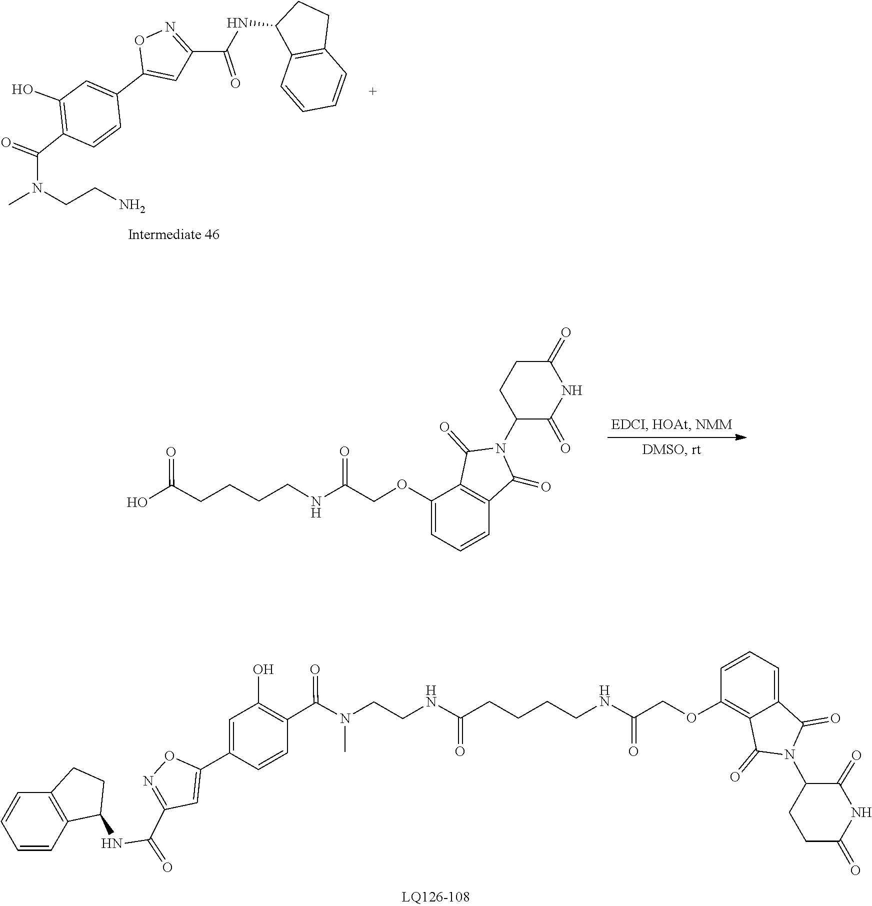 [SMILES: CN(CCN)C(=O)c1ccc(-c2cc(C(=O)N[C@@H]3CCc4ccccc43)no2)cc1O ; O=C(O)CCCCNC(=O)COc1cccc2c1C(=O)N(C1CCC(=O)NC1=O)C2=O ; CN(CCNC(=O)CCCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O]

LQ126-108 was synthesized following the standard procedure for preparing LQ126-89 from intermediate 46 (4 mg, 0.01 mmol), 5-(2-((2-(2,6-dioxopiperidin-3-yl)-1,3-dioxoisoindolin-4-yl)oxy)acetamido)pentanoic acid (4.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-108 was obtained as white solid (5 mg, 60%). ¹H NMR (600 MHz, Methanol-d₄) δ 7.79 (t, J=7.9 Hz, 1H), 7.55-7.49 (m, 1H), 7.44-7.37 (m, 2H), 7.36-7.21 (m, 6H), 7.16-7.10 (m, 1H), 5.66 (t, J=7.3 Hz, 1H), 5.14 (ddd, J=12.8, 5.5, 3.0 Hz, 1H), 4.75 (s, 2H), 3.74-3.65 (m, 1H), 3.56-3.48 (m, 1H), 3.45-3.38 (m, 2H), 3.19-3.07 (m, 3H), 3.03-2.83 (m, 5H), 2.81-2.69 (m, 2H), 2.66-2.58 (m, 1H), 2.30-2.23 (m, 1H), 2.20-2.04 (m, 3H), 1.76-1.53 (m, 4H). HRMS m/z [M+H]⁺ calcd for C₄₃H₄₄N₇O₁₁⁺ 834.3093, found 834.3083.

### Example 248

### Synthesis of LQ126-109

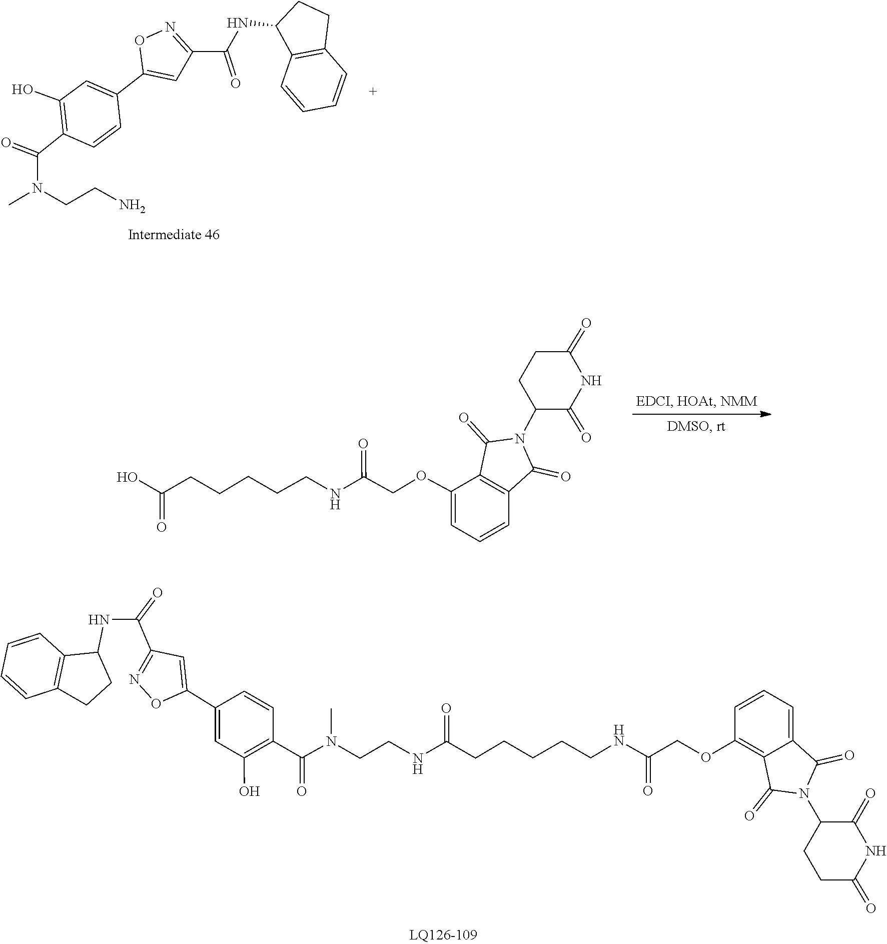 [SMILES: CN(CCN)C(=O)c1ccc(-c2cc(C(=O)N[C@@H]3CCc4ccccc43)no2)cc1O ; O=C(O)CCCCCNC(=O)COc1cccc2c1C(=O)N(C1CCC(=O)NC1=O)C2=O ; CN(CCNC(=O)CCCCCNC(=O)COc1cccc2c1C(=O)N(C1CCC(=O)NC1=O)C2=O)C(=O)c1ccc(-c2cc(C(=O)NC3CCc4ccccc43)no2)cc1O]

LQ126-109 was synthesized following the standard procedure for preparing LQ126-89 from intermediate 46 (4 mg, 0.01 mmol), 6-(2-((2-(2,6-dioxopiperidin-3-yl)-1,3-dioxoisoindolin-4-yl)oxy)acetamido)hexanoic acid (4.4 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-109 was obtained as white solid (5.6 mg, 67%). ¹H NMR (600 MHz, Methanol-d₄) δ 7.79 (t, J=7.9 Hz, 1H), 7.52 (d, J=7.3 Hz, 1H), 7.43-7.39 (m, 2H), 7.35-7.18 (m, 6H), 7.12 (s, 1H), 5.66-5.60 (m, 1H), 5.15-5.11 (m, 1H), 4.74 (s, 2H), 3.73-3.65 (m, 1H), 3.54-3.46 (m, 1H), 3.44-3.36 (m, 2H), 3.15-3.06 (m, 3H), 3.02-2.82 (m, 5H), 2.80-2.68 (m, 2H), 2.63-2.55 (m, 1H), 2.26-2.02 (m, 4H), 1.71-1.49 (m, 4H), 1.43-1.27 (m, 2H). HRMS m/z [M+H]⁺ calcd for C₄₄H₄₆N₇O₁₁⁺ 848.3250, found 848.3245.

### Example 249

### Synthesis of LQ126-110

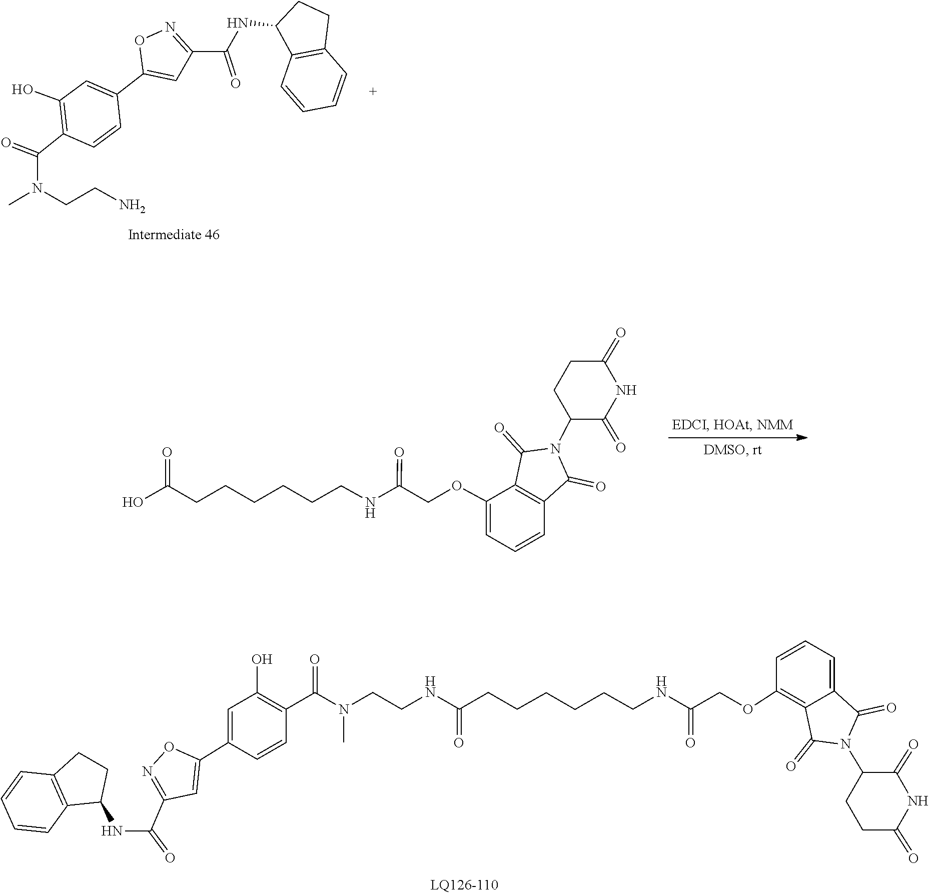 [SMILES: CN(CCN)C(=O)c1ccc(-c2cc(C(=O)N[C@@H]3CCc4ccccc43)no2)cc1O ; O=C(O)CCCCCCNC(=O)COc1cccc2c1C(=O)N(C1CCC(=O)NC1=O)C2=O ; CN(CCNC(=O)CCCCCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O]

LQ126-110 was synthesized following the standard procedure for preparing LQ126-89 from intermediate 46 (4 mg, 0.01 mmol), 7-(2-((2-(2,6-dioxopiperidin-3-yl)-1,3-dioxoisoindolin-4-yl)oxy)acetamido)heptanoic acid (4.6 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-110 was obtained as white solid (6 mg, 70%). ¹H NMR (600 MHz, Methanol-d₄) δ 7.79 (t, J=7.9 Hz, 1H), 7.52 (d, J=7.3 Hz, 1H), 7.43-7.38 (m, 2H), 7.36-7.18 (m, 6H), 7.12 (s, 1H), 5.63 (t, J=7.6 Hz, 1H), 5.17-5.08 (m, 1H), 4.73 (s, 2H), 3.73-3.65 (m, 1H), 3.57-3.47 (m, 1H), 3.44-3.36 (m, 2H), 3.15-3.05 (m, 3H), 3.03-2.82 (m, 4H), 2.79-2.67 (m, 2H), 2.63-2.55 (m, 1H), 2.24-2.01 (m, 5H), 1.71-1.49 (m, 4H), 1.44-1.25 (m, 4H). HRMS m/z [M+H]⁺ calcd for C₄₅H₄₈N₇O₁₁⁺ 862.3406, found 862.3403.

### Example 250

### Synthesis of LQ126-112

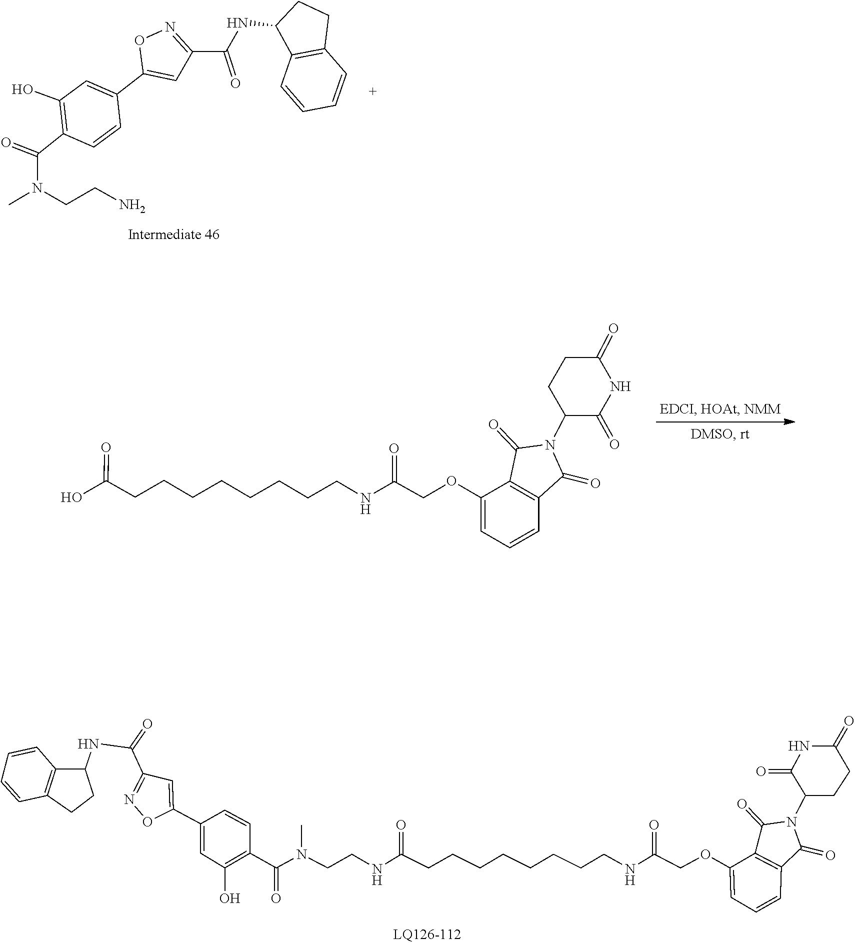 [SMILES: CN(CCN)C(=O)c1ccc(-c2cc(C(=O)N[C@@H]3CCc4ccccc43)no2)cc1O ; O=C(O)CCCCCCCCNC(=O)COc1cccc2c1C(=O)N(C1CCC(=O)NC1=O)C2=O ; CN(CCNC(=O)CCCCCCCCNC(=O)COc1cccc2c1C(=O)N(C1CCC(=O)NC1=O)C2=O)C(=O)c1ccc(-c2cc(C(=O)NC3CCc4ccccc43)no2)cc1O]

LQ126-112 was synthesized following the standard procedure for preparing LQ126-89 from intermediate 46 (4 mg, 0.01 mmol), 9-(2-((2-(2,6-dioxopiperidin-3-yl)-1,3-dioxoisoindolin-4-yl)oxy)acetamido)nonanoic acid (4.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-112 was obtained as white solid (6.5 mg, 73%). ¹H NMR (600 MHz, Methanol-d₄) δ 7.81 (t, J=7.9 Hz, 1H), 7.54 (d, J=7.3 Hz, 1H), 7.45-7.40 (m, 2H), 7.39-7.31 (m, 3H), 7.30-7.19 (m, 3H), 7.15 (s, 1H), 5.65 (t, J=7.7 Hz, 1H), 5.14 (dd, J=12.6, 5.5 Hz, 1H), 4.75 (s, 2H), 3.75-3.66 (m, 1H), 3.59-3.50 (m, 1H), 3.47-3.38 (m, 2H), 3.17-3.06 (m, 3H), 3.04-2.84 (m, 4H), 2.81-2.70 (m, 2H), 2.64-2.57 (m, 1H), 2.25-2.03 (m, 5H), 1.69-1.48 (m, 4H), 1.41-1.24 (m, 8H). HRMS m/z [M+H]⁺ calcd for C₄₇H₅₂N₇O₁₁⁺ 890.3719, found 890.3695.

### Example 251

### Synthesis of LQ126-113

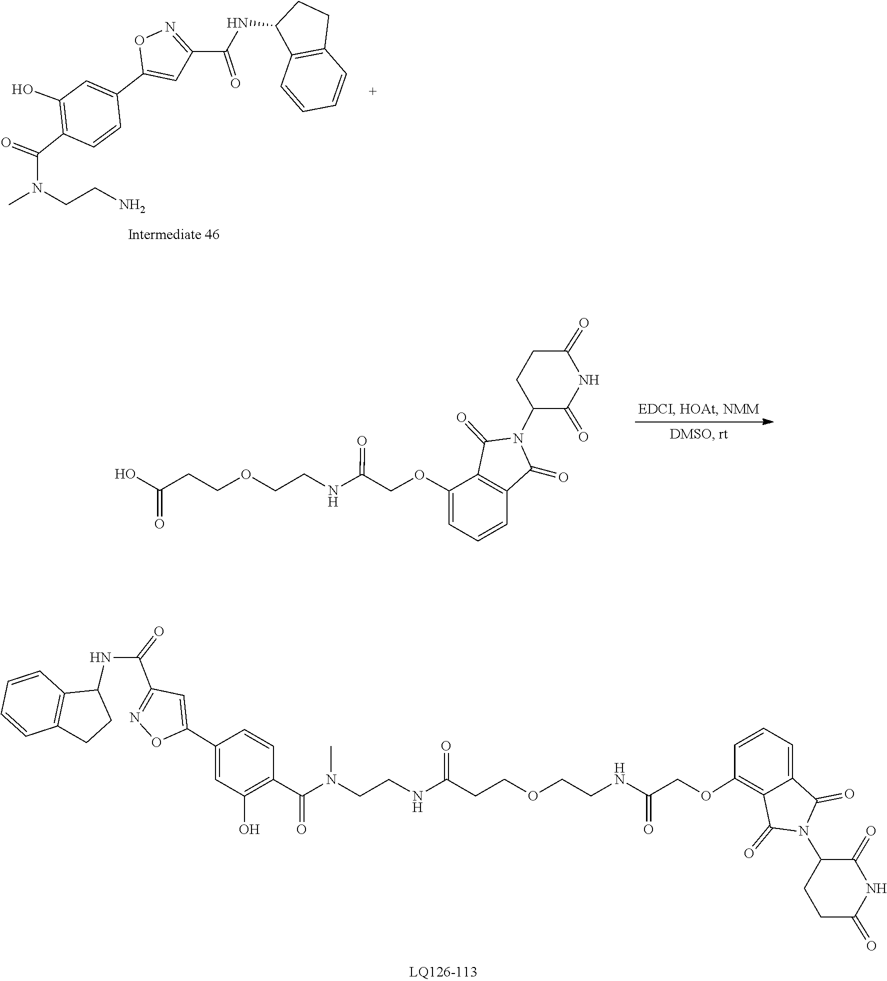 [SMILES: CN(CCN)C(=O)c1ccc(-c2cc(C(=O)N[C@@H]3CCc4ccccc43)no2)cc1O ; O=C(O)CCOCCNC(=O)COc1cccc2c1C(=O)N(C1CCC(=O)NC1=O)C2=O ; CN(CCNC(=O)CCOCCNC(=O)COc1cccc2c1C(=O)N(C1CCC(=O)NC1=O)C2=O)C(=O)c1ccc(-c2cc(C(=O)NC3CCc4ccccc43)no2)cc1O]

LQ126-113 was synthesized following the standard procedure for preparing LQ126-89 from intermediate 46 (4 mg, 0.01 mmol), 3-(2-(2-((2-(2,6-dioxopiperidin-3-yl)-1,3-dioxoisoindolin-4-yl)oxy)acetamido)ethoxy)propanoic acid (4.5 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-113 was obtained as white solid (5.2 mg, 62%). ¹H NMR (600 MHz, Methanol-d₄) δ 7.77 (t, J=7.9 Hz, 1H), 7.50 (d, J=7.3 Hz, 1H), 7.40-7.35 (m, 2H), 7.35-7.19 (m, 6H), 7.10 (s, 1H), 5.67-5.61 (m, 1H), 5.17-5.08 (m, 1H), 4.73 (s, 2H), 3.80-3.71 (m, 1H), 3.70-3.63 (m, 2H), 3.62-3.45 (m, 5H), 3.40-3.33 (m, 2H), 3.13-3.05 (m, 3H), 3.00-2.82 (m, 4H), 2.78-2.66 (m, 2H), 2.64-2.56 (m, 1H), 2.52-2.35 (m, 2H), 2.18-2.10 (m, 1H), 2.10-2.01 (m, 1H). HRMS m/z [M+H]⁺ calcd for C₄₃H₄₄N₇O₁₂⁺ 850.3042, found 850.3037.

### Example 252

### Synthesis of LQ126-114

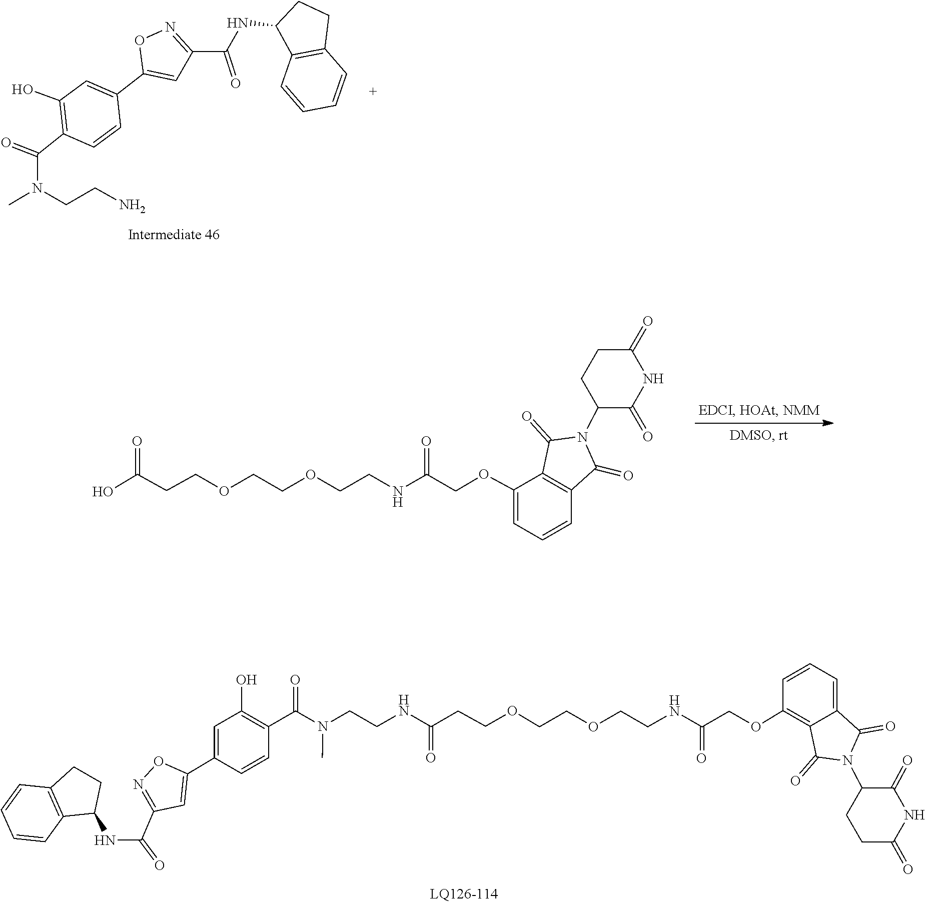 [SMILES: CN(CCN)C(=O)c1ccc(-c2cc(C(=O)N[C@@H]3CCc4ccccc43)no2)cc1O ; O=C(O)CCOCCOCCNC(=O)COc1cccc2c1C(=O)N(C1CCC(=O)NC1=O)C2=O ; CN(CCNC(=O)CCOCCOCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O]

LQ126-114 was synthesized following the standard procedure for preparing LQ126-89 from intermediate 46 (4 mg, 0.01 mmol), 3-(2-(2-(2-((2-(2,6-dioxopiperidin-3-yl)-1,3-dioxoisoindolin-4-yl)oxy)acetamido)ethoxy)ethoxy)propanoic acid (4.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-114 was obtained as white solid (5.1 mg, 57%). ¹H NMR (600 MHz, Methanol-d₄) δ 7.80 (t, J=7.9 Hz, 1H), 7.53 (d, J=7.3 Hz, 1H), 7.44-7.40 (m, 2H), 7.38-7.31 (m, 3H), 7.31-7.20 (m, 3H), 7.13 (s, 1H), 5.65 (t, J=7.6 Hz, 1H), 5.18-5.10 (m, 1H), 4.77 (s, 2H), 3.79-3.57 (m, 8H), 3.55-3.45 (m, 3H), 3.44-3.38 (m, 2H), 3.15-3.07 (m, 3H), 3.04-2.85 (m, 4H), 2.81-2.69 (m, 2H), 2.65-2.57 (m, 1H), 2.50-2.35 (m, 2H), 2.20-2.12 (m, 1H), 2.12-2.04 (m, 1H). HRMS m/z [M+H]⁺ calcd for C₄₅H₄₈N₇O₁₃⁺ 894.3305, found 894.3297.

### Example 253

### Synthesis of LQ126-115

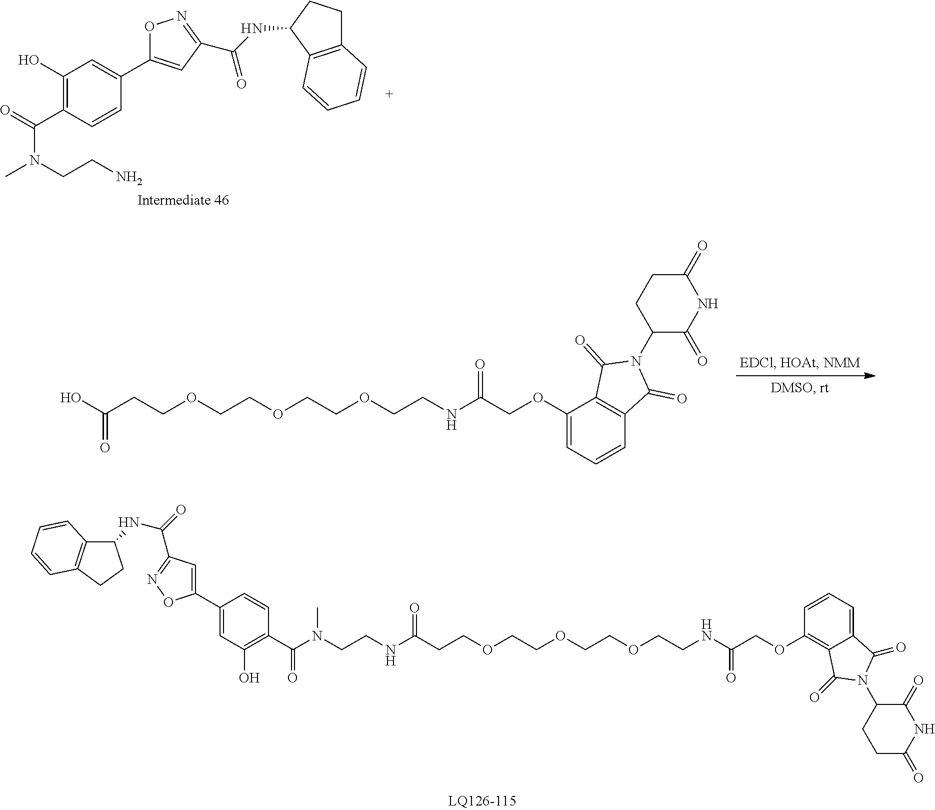 [SMILES: CN(CCN)C(=O)c1ccc(-c2cc(C(=O)N[C@@H]3CCc4ccccc43)no2)cc1O ; O=C(O)CCOCCOCCOCCNC(=O)COc1cccc2c1C(=O)N(C1CCC(=O)NC1=O)C2=O ; CN(CCNC(=O)CCOCCOCCOCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O]

LQ126-115 was synthesized following the standard procedure for preparing LQ126-89 from intermediate 46 (4 mg, 0.01 mmol), 1-((2-(2,6-dioxopiperidin-3-yl)-1,3-dioxoisoindolin-4-yl)oxy)-2-oxo-6,9,12-trioxa-3-azapentadecan-15-oic acid (5.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-115 was obtained as white solid (5.6 mg, 60%). ¹H NMR (600 MHz, Methanol-d₄) δ 7.81 (t, J=7.9 Hz, 1H), 7.54 (d, J=7.3 Hz, 1H), 7.45-7.41 (m, 2H), 7.39-7.32 (m, 3H), 7.31-7.20 (m, 3H), 7.14 (s, 1H), 5.65 (t, J=7.6 Hz, 1H), 5.16-5.11 (m, 1H), 4.77 (s, 2H), 3.78-3.48 (m, 14H), 3.44-3.36 (m, 3H), 3.17-3.07 (m, 3H), 3.04-2.85 (m, 4H), 2.80-2.67 (m, 2H), 2.65-2.57 (m, 1H), 2.51-2.36 (m, 2H), 2.19-2.13 (m, 1H), 2.12-2.03 (m, 1H). HRMS m/z [M+H]⁺ calcd for C₄₇H₅₂N₇O₁₄⁺ 938.3567, found 938.3570.

### Example 254

### Synthesis of LQ126-116

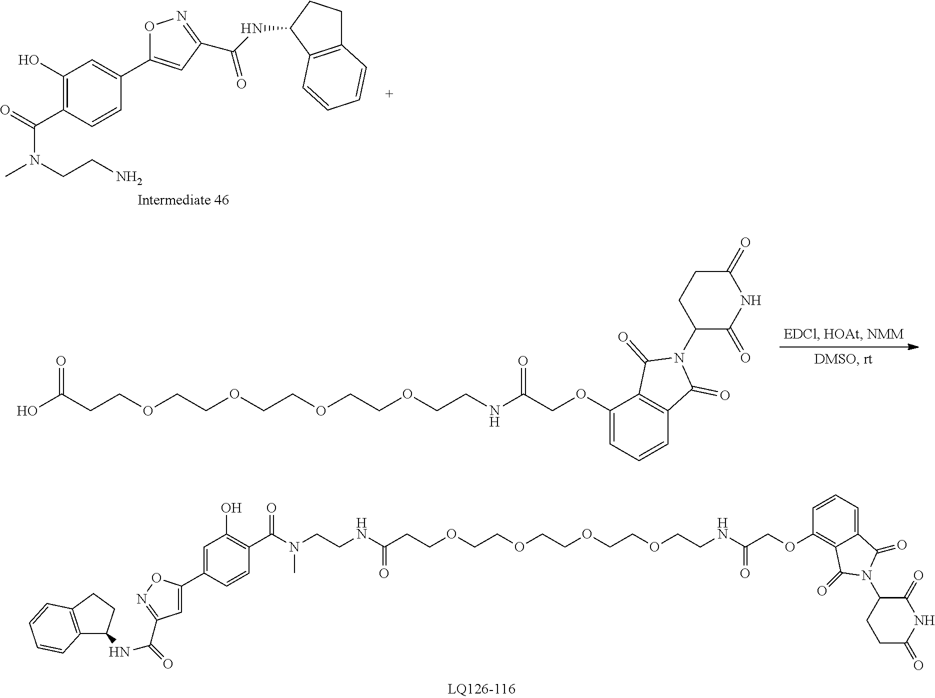 [SMILES: CN(CCN)C(=O)c1ccc(-c2cc(C(=O)N[C@@H]3CCc4ccccc43)no2)cc1O ; O=C(O)CCOCCOCCOCCOCCNC(=O)COc1cccc2c1C(=O)N(C1CCC(=O)NC1=O)C2=O ; CN(CCNC(=O)CCOCCOCCOCCOCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O]

LQ126-116 was synthesized following the standard procedure for preparing LQ126-89 from intermediate 46 (4 mg, 0.01 mmol), 1-((2-(2,6-dioxopiperidin-3-yl)-1,3-dioxoisoindolin-4-yl)oxy)-2-oxo-6,9,12,15-tetraoxa-3-azaoctadecan-18-oic acid (5.8 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-116 was obtained as white solid (6.5 mg, 66%). ¹H NMR (600 MHz, Methanol-d₄) δ 7.79 (t, J=7.9 Hz, 1H), 7.52 (d, J=7.3 Hz, 1H), 7.43-7.39 (m, 2H), 7.36-7.29 (m, 3H), 7.28-7.18 (m, 3H), 7.13 (s, 1H), 5.63 (t, J=7.7 Hz, 1H), 5.15-5.09 (m, 1H), 4.75 (s, 2H), 3.75-3.45 (m, 18H), 3.44-3.34 (m, 3H), 3.16-3.05 (m, 3H), 3.02-2.82 (m, 4H), 2.78-2.68 (m, 2H), 2.62-2.56 (m, 1H), 2.49-2.34 (m, 2H), 2.17-2.12 (m, 1H), 2.09-2.02 (m, 1H). HRMS m/z [M+H]⁺ calcd for C₄₉H₅₆N₇O₁₅⁺ 982.3829, found 982.3830.

### Example 255

### Synthesis of LQ126-117

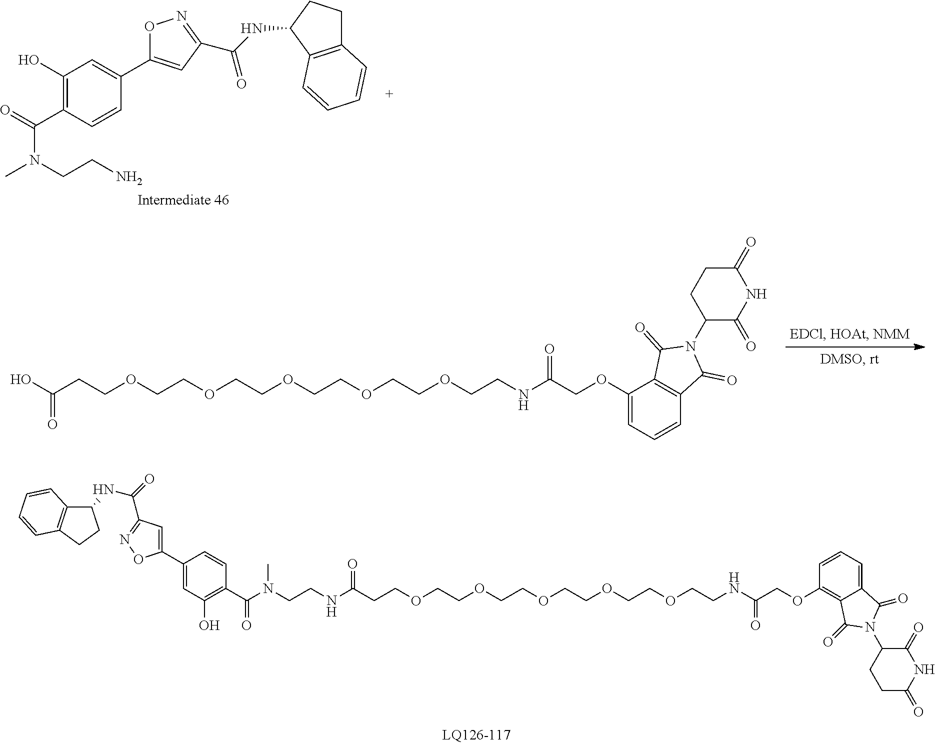 [SMILES: CN(CCN)C(=O)c1ccc(-c2cc(C(=O)N[C@@H]3CCc4ccccc43)no2)cc1O ; O=C(O)CCOCCOCCOCCOCCOCCNC(=O)COc1cccc2c1C(=O)N(C1CCC(=O)NC1=O)C2=O ; CN(CCNC(=O)CCOCCOCCOCCOCCOCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O]

LQ126-117 was synthesized following the standard procedure for preparing LQ126-89 from intermediate 46 (4 mg, 0.01 mmol), 1-((2-(2,6-dioxopiperidin-3-yl)-1,3-dioxoisoindolin-4-yl)oxy)-2-oxo-6,9,12,15,18-pentaoxa-3-azahenicosan-21-oic acid (6.2 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-117 was obtained as white solid (6.4 mg, 63%). ¹H NMR (600 MHz, Methanol-d₄) δ 7.81 (t, J=7.9 Hz, 1H), 7.54 (d, J=7.3 Hz, 1H), 7.46-7.41 (m, 2H), 7.39-7.32 (m, 3H), 7.30-7.20 (m, 3H), 7.15 (s, 1H), 5.65 (t, J=7.8 Hz, 1H), 5.14 (ddd, J=12.9, 5.6, 2.1 Hz, 1H), 4.78 (s, 2H), 3.79-3.48 (m, 22H), 3.44-3.36 (m, 3H), 3.17-3.07 (m, 3H), 3.04-2.85 (m, 4H), 2.81-2.70 (m, 2H), 2.64-2.57 (m, 1H), 2.52-2.36 (m, 2H), 2.19-2.13 (m, 1H), 2.12-2.03 (m, 1H). HRMS m/z [M+H]⁺ calcd for C₅₁H₆₀N₇O₁₆⁺ 1026.4091, found 1026.4097.

### Example 256

### Synthesis of LQ126-118

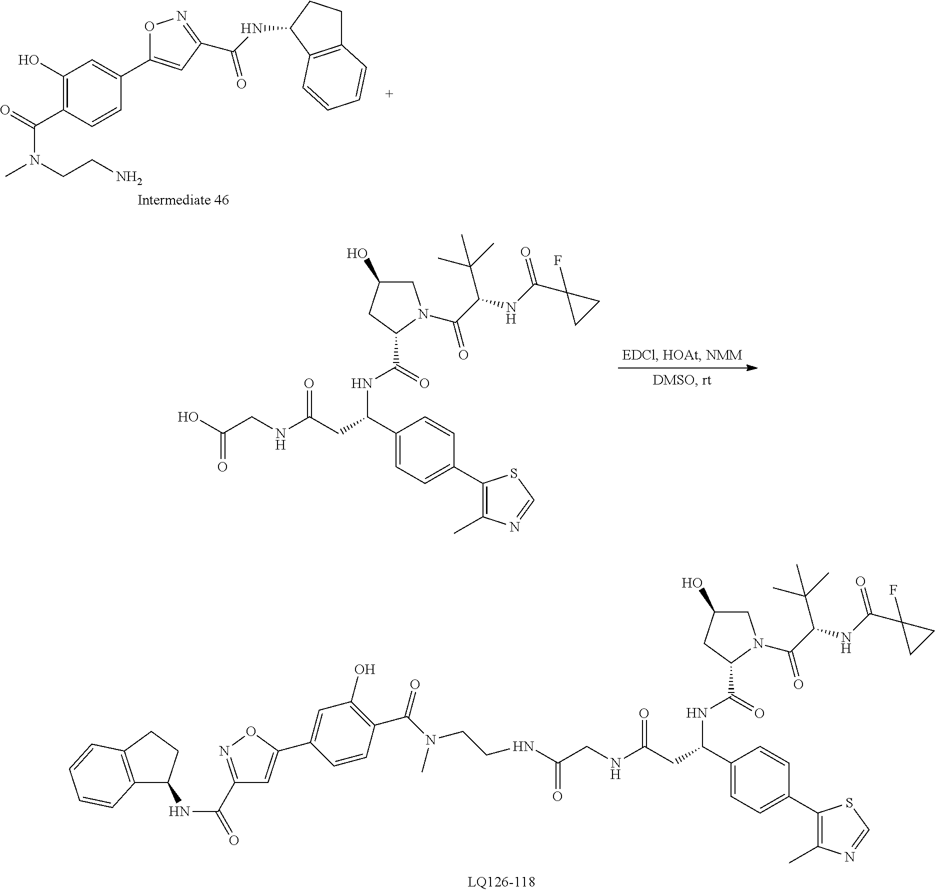 [SMILES: CN(CCN)C(=O)c1ccc(-c2cc(C(=O)N[C@@H]3CCc4ccccc43)no2)cc1O ; Cc1ncsc1-c1ccc([C@H](CC(=O)NCC(=O)O)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1]

LQ126-118 was synthesized following the standard procedure for preparing LQ126-89 from intermediate 46 (4 mg, 0.01 mmol), ((S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanoyl)glycine (6.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-118 was obtained as white solid (6.3 mg, 61%). ¹H NMR (600 MHz, Methanol-d₄) δ 9.04 (s, 1H), 7.51-7.20 (m, 11H), 7.13 (s, 1H), 5.66 (t, J=7.9 Hz, 1H), 5.45-5.37 (m, 1H), 4.76 (d, J=9.1 Hz, 1H), 4.68-4.56 (m, 1H), 4.47 (s, 1H), 3.89-3.73 (m, 2H), 3.60-3.51 (m, 1H), 3.44-3.37 (m, 1H), 3.18-3.06 (m, 2H), 3.03-2.83 (m, 7H), 2.65-2.57 (m, 1H), 2.48 (s, 3H), 2.23 (dd, J=13.2, 7.6 Hz, 1H), 2.11-2.04 (m, 2H), 2.02-1.97 (m, 2H), 1.41-1.24 (m, 4H), 1.07 (s, 9H). HRMS m/z [M+H]⁺ calcd for C₅₃H₆₁FN₉O₁₀S⁺ 1034.4241, found 1034.4245.

### Example 257

### Synthesis of LQ126-120

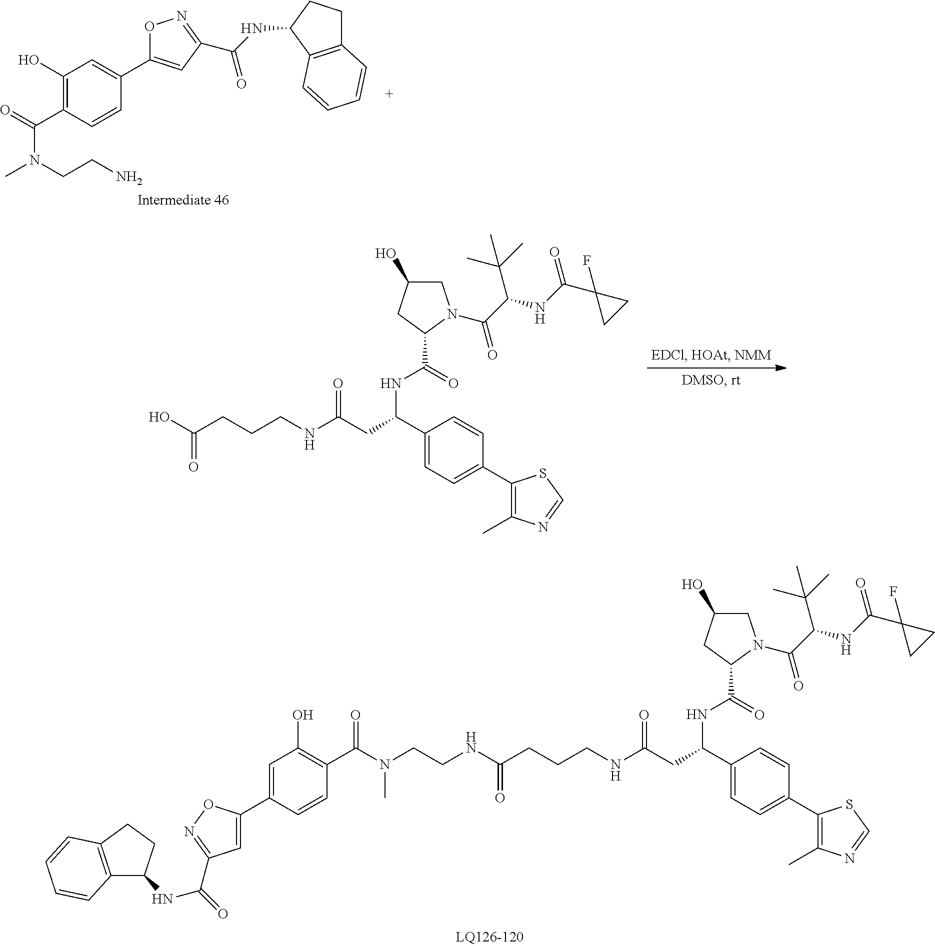 [SMILES: CN(CCN)C(=O)c1ccc(-c2cc(C(=O)N[C@@H]3CCc4ccccc43)no2)cc1O ; Cc1ncsc1-c1ccc([C@H](CC(=O)NCCCC(=O)O)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1]

LQ126-120 was synthesized following the standard procedure for preparing LQ126-89 from intermediate 46 (4 mg, 0.01 mmol), 4-((S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanamido)butanoic acid (6.6 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-120 was obtained as white solid (5.8 mg, 55%). ¹H NMR (600 MHz, Methanol-d₄) δ 9.07 (s, 1H), 7.52-7.39 (m, 5H), 7.36-7.18 (m, 6H), 7.13 (s, 1H), 5.64 (t, J=7.8 Hz, 1H), 5.35-5.29 (m, 1H), 4.73 (d, J=9.2 Hz, 1H), 4.62-4.55 (m, 1H), 4.44 (s, 1H), 3.82 (d, J=11.1 Hz, 1H), 3.76 (dd, J=11.1, 3.8 Hz, 1H), 3.70-3.62 (m, 1H), 3.50-3.43 (m, 1H), 3.18-3.04 (m, 6H), 3.01-2.90 (m, 4H), 2.84 (dd, J=14.2, 6.4 Hz, 1H), 2.74 (dd, J=14.2, 8.1 Hz, 1H), 2.62-2.56 (m, 1H), 2.48 (s, 3H), 2.19 (dd, J=13.4, 7.7 Hz, 1H), 2.12-1.91 (m, 3H), 1.76-1.53 (m, 2H), 1.39-1.24 (m, 4H), 1.05 (s, 9H). HRMS m/z [M+H]⁺ calcd for C₅₅H₆₅FN₉O₁₀S⁺ 1062.4554, found 1062.4547.

### Example 258

### Synthesis of LQ126-121

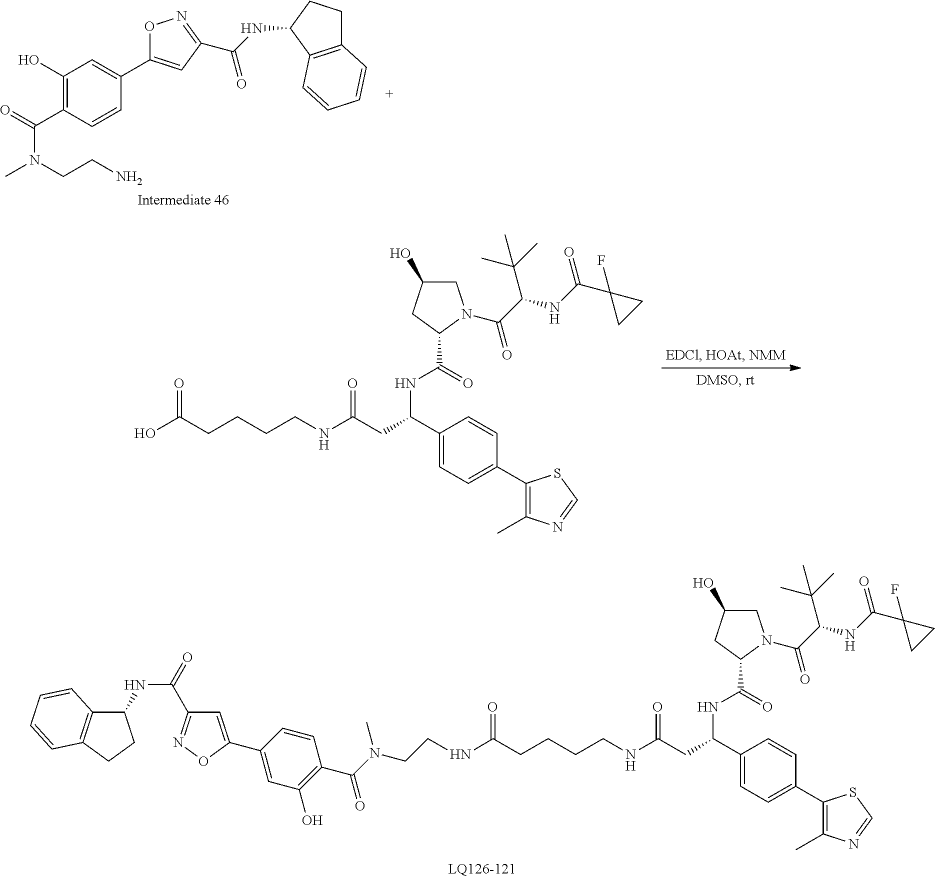 [SMILES: CN(CCN)C(=O)c1ccc(-c2cc(C(=O)N[C@@H]3CCc4ccccc43)no2)cc1O ; Cc1ncsc1-c1ccc([C@H](CC(=O)NCCCCC(=O)O)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1]

LQ126-121 was synthesized following the standard procedure for preparing LQ126-89 from intermediate 46 (4 mg, 0.01 mmol), 5-((S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanamido)pentanoic acid (6.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-121 was obtained as white solid (6.2 mg, 58%). ¹H NMR (600 MHz, Methanol-d₄) δ 9.08 (s, 1H), 7.51-7.41 (m, 5H), 7.40-7.28 (m, 4H), 7.28-7.20 (m, 2H), 7.15 (s, 1H), 5.66 (t, J=7.7 Hz, 1H), 5.32 (dd, J=8.1, 6.2 Hz, 1H), 4.75 (d, J=9.2 Hz, 1H), 4.61 (t, J=8.8 Hz, 1H), 4.46 (s, 1H), 3.84 (d, J=11.1 Hz, 1H), 3.78 (dd, J=11.1, 3.8 Hz, 1H), 3.72-3.63 (m, 1H), 3.55-3.45 (m, 1H), 3.18-3.05 (m, 6H), 3.03-2.91 (m, 4H), 2.87-2.81 (m, 1H), 2.75 (dd, J=14.2, 8.2 Hz, 1H), 2.66-2.57 (m, 1H), 2.50 (s, 3H), 2.23-2.14 (m, 1H), 2.11-2.03 (m, 2H), 2.00-1.93 (m, 1H), 1.60-1.50 (m, 2H), 1.49-1.28 (m, 6H), 1.07 (s, 9H). HRMS m/z [M+H]⁺ calcd for C₅₆H₆₇FN₉O₁₀S⁺ 1076.4710, found 1076.4713.

### Example 259

### Synthesis of LQ126-122

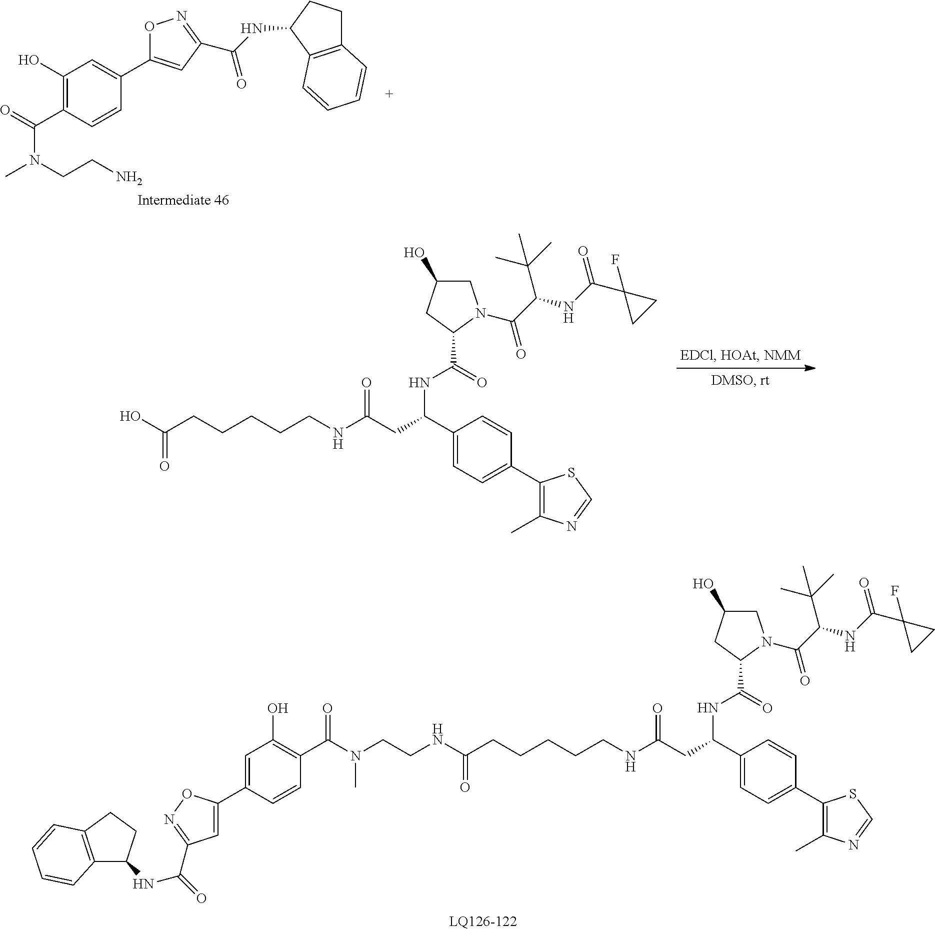 [SMILES: CN(CCN)C(=O)c1ccc(-c2cc(C(=O)N[C@@H]3CCc4ccccc43)no2)cc1O ; Cc1ncsc1-c1ccc([C@H](CC(=O)NCCCCCC(=O)O)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1]

LQ126-122 was synthesized following the standard procedure for preparing LQ126-89 from intermediate 46 (4 mg, 0.01 mmol), 6-((S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanamido)hexanoic acid (6.8 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-122 was obtained as white solid (6.6 mg, 61%). ¹H NMR (600 MHz, Methanol-d₄) δ 9.03 (s, 1H), 7.51-7.39 (m, 5H), 7.38-7.26 (m, 4H), 7.25-7.18 (m, 2H), 7.13 (s, 1H), 5.64 (t, J=7.7 Hz, 1H), 5.30 (dd, J=8.1, 6.2 Hz, 1H), 4.73 (d, J=9.2 Hz, 1H), 4.58 (t, J=8.5 Hz, 1H), 4.44 (s, 1H), 3.82 (d, J=11.1 Hz, 1H), 3.76 (dd, J=11.1, 3.8 Hz, 1H), 3.73-3.65 (m, 1H), 3.50-3.47 (m, 1H), 3.15-3.03 (m, 6H), 3.00-2.89 (m, 4H), 2.83 (dd, J=14.1, 6.2 Hz, 1H), 2.73 (dd, J=14.3, 8.2 Hz, 1H), 2.64-2.55 (m, 1H), 2.48 (s, 3H), 2.22-2.14 (m, 1H), 2.10-2.01 (m, 2H), 1.98-1.91 (m, 1H), 1.62-1.43 (m, 2H), 1.42-1.14 (m, 8H), 1.05 (s, 9H). HRMS m/z [M+H]⁺ calcd for C₅₇H₆₉FN₉O₁₀S⁺ 1090.4867, found 1090.4872.

### Example 260

### Synthesis of LQ126-123

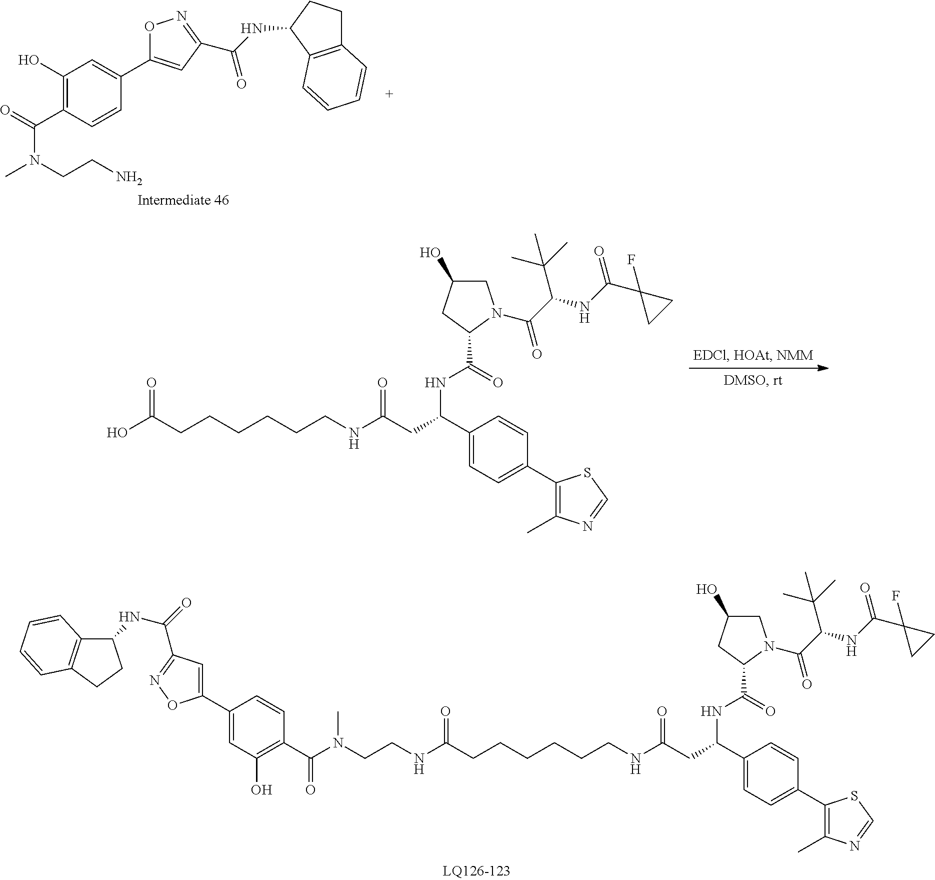 [SMILES: CN(CCN)C(=O)c1ccc(-c2cc(C(=O)N[C@@H]3CCc4ccccc43)no2)cc1O ; Cc1ncsc1-c1ccc([C@H](CC(=O)NCCCCCCC(=O)O)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1]

LQ126-123 was synthesized following the standard procedure for preparing LQ126-89 from intermediate 46 (4 mg, 0.01 mmol), 7-((S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanamido)heptanoic acid (7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-123 was obtained as white solid (7.2 mg, 65%). ¹H NMR (600 MHz, Methanol-d₄) δ 9.06 (s, 1H), 7.50-7.38 (m, 4H), 7.37-7.18 (m, 7H), 7.13 (s, 1H), 5.64 (t, J=7.8 Hz, 1H), 5.30 (dd, J=8.3, 6.0 Hz, 1H), 4.73 (d, J=9.2 Hz, 1H), 4.58 (t, J=8.5 Hz, 1H), 4.44 (s, 1H), 3.82 (d, J=11.1 Hz, 1H), 3.76 (dd, J=11.1, 3.8 Hz, 1H), 3.72-3.65 (m, 1H), 3.52-3.46 (m, 1H), 3.15-2.90 (m, 10H), 2.83 (dd, J=14.1, 6.0 Hz, 1H), 2.73 (dd, J=14.0, 8.4 Hz, 1H), 2.64-2.55 (m, 1H), 2.49 (s, 3H), 2.21-2.12 (m, 1H), 2.09-2.02 (m, 2H), 1.98-1.91 (m, 1H), 1.60-1.51 (m, 1H), 1.49-1.40 (m, 1H), 1.38-1.13 (m, 10H), 1.05 (s, 9H). HRMS m/z [M+H]⁺ calcd for C₅₈H₇₁FN₉O₁₀S⁺ 1104.5023, found 1104.5034.

### Example 261

### Synthesis of LQ126-124

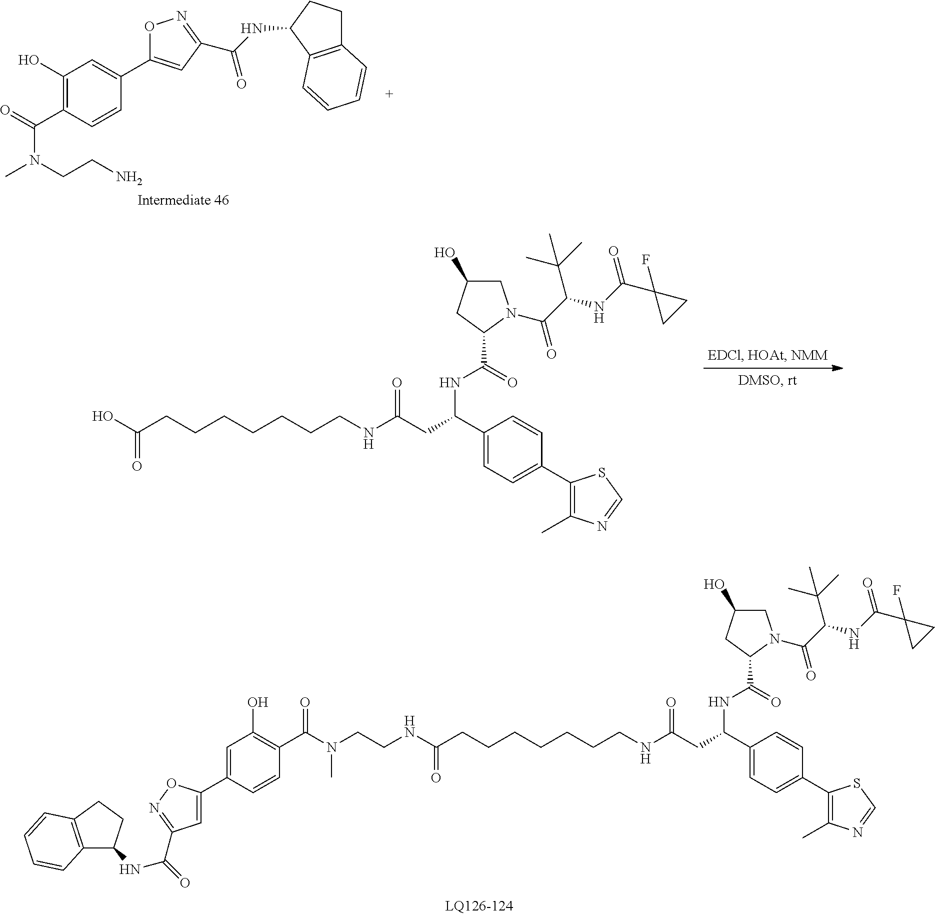 [SMILES: CN(CCN)C(=O)c1ccc(-c2cc(C(=O)N[C@@H]3CCc4ccccc43)no2)cc1O ; Cc1ncsc1-c1ccc([C@H](CC(=O)NCCCCCCCC(=O)O)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCCCCCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1]

LQ126-124 was synthesized following the standard procedure for preparing LQ126-89 from intermediate 46 (4 mg, 0.01 mmol), 8-((S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanamido)octanoic acid (7.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-124 was obtained as white solid (5.9 mg, 53%). ¹H NMR (600 MHz, Methanol-d₄) δ 9.00 (s, 1H), 7.49-7.38 (m, 4H), 7.38-7.17 (m, 6H), 7.13 (s, 1H), 5.64 (t, J=7.7 Hz, 1H), 5.30 (dd, J=8.3, 6.0 Hz, 1H), 4.73 (d, J=9.3 Hz, 1H), 4.58 (dd, J=9.2, 7.7 Hz, 1H), 4.44 (s, 1H), 3.82 (d, J=11.1 Hz, 1H), 3.76 (dd, J=11.1, 3.8 Hz, 1H), 3.71-3.56 (m, 4H), 3.50 (d, J=5.8 Hz, 2H), 3.23-3.18 (m, 1H), 3.17-2.88 (m, 7H), 2.83 (dd, J=14.1, 5.9 Hz, 1H), 2.73 (dd, J=14.1, 8.4 Hz, 1H), 2.62-2.56 (m, 1H), 2.48 (s, 3H), 2.21-2.12 (m, 2H), 2.09-2.02 (m, 1H), 1.98-1.92 (m, 1H), 1.63-1.50 (m, 1H), 1.48-1.42 (m, 3H), 1.38-1.11 (m, 8H), 1.06 (s, 9H). HRMS m/z [M+H]⁺ calcd for C₅₉H₇₃FN₉O₁₀S⁺ 1118.5180, found 1118.5198.

### Example 262

### Synthesis of LQ126-125

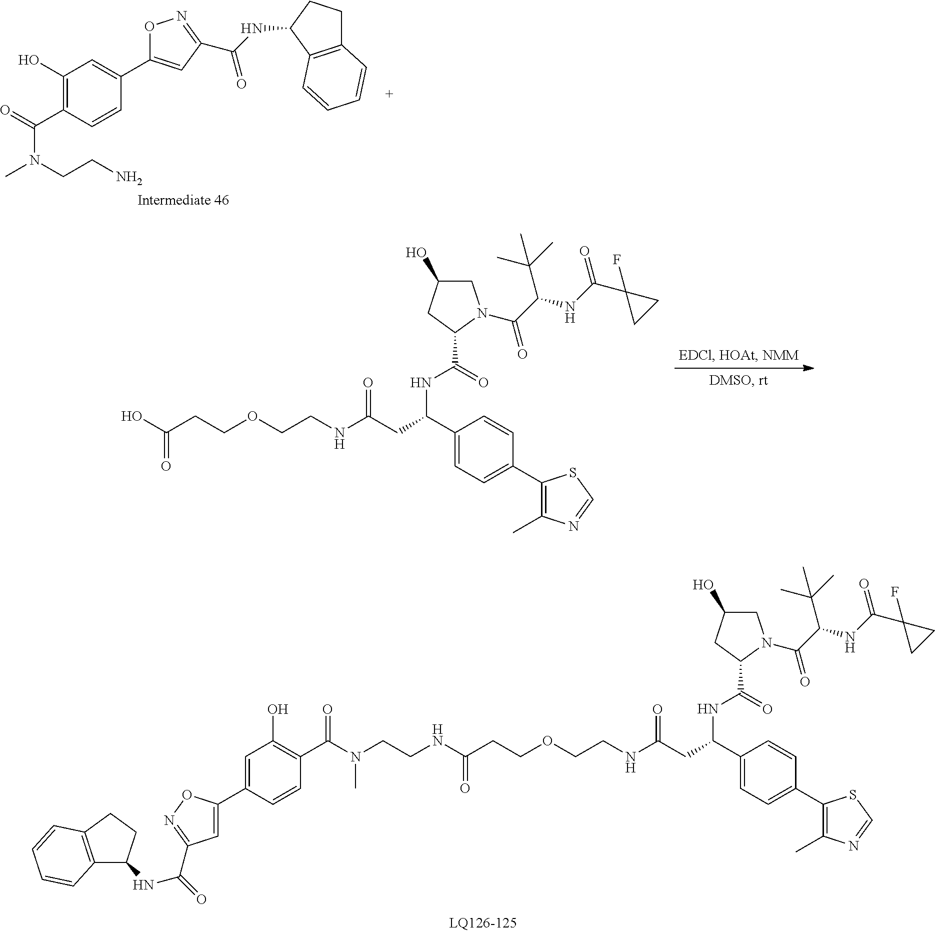 [SMILES: CN(CCN)C(=O)c1ccc(-c2cc(C(=O)N[C@@H]3CCc4ccccc43)no2)cc1O ; Cc1ncsc1-c1ccc([C@H](CC(=O)NCCOCCC(=O)O)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCOCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1]

LQ126-125 was synthesized following the standard procedure for preparing LQ126-89 from intermediate 46 (4 mg, 0.01 mmol), 3-(2-((S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanamido)ethoxy)propanoic acid (6.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-125 was obtained as white solid (6.2 mg, 57%). ¹H NMR (600 MHz, Methanol-d₄) δ 8.96 (s, 1H), 7.51 (dd, J=9.3, 3.4 Hz, 1H), 7.47-7.35 (m, 6H), 7.34-7.20 (m, 4H), 7.14 (s, 1H), 5.66 (t, J=7.7 Hz, 1H), 5.33 (t, J=7.1 Hz, 1H), 4.75 (d, J=9.3 Hz, 1H), 4.63-4.57 (m, 1H), 4.45 (s, 1H), 3.83 (d, J=11.1 Hz, 1H), 3.77 (dd, J=11.1, 3.8 Hz, 1H), 3.72-3.57 (m, 3H), 3.56-3.51 (m, 1H), 3.49-3.36 (m, 3H), 3.18-3.07 (m, 3H), 3.03-2.91 (m, 4H), 2.85 (dd, J=14.2, 6.8 Hz, 1H), 2.76 (dd, J=14.2, 7.7 Hz, 1H), 2.65-2.58 (m, 1H), 2.48 (s, 3H), 2.47-2.33 (m, 2H), 2.24-2.16 (m, 1H), 2.12-2.04 (m, 1H), 2.00-1.93 (m, 1H), 1.42-1.26 (m, 6H), 1.07 (s, 9H). HRMS m/z [M+H]⁺ calcd for C₅₆H₆₇FN₉O₁₁S⁺ 1092.4659, found 1092.4672.

### Example 263

### Synthesis of LQ126-126

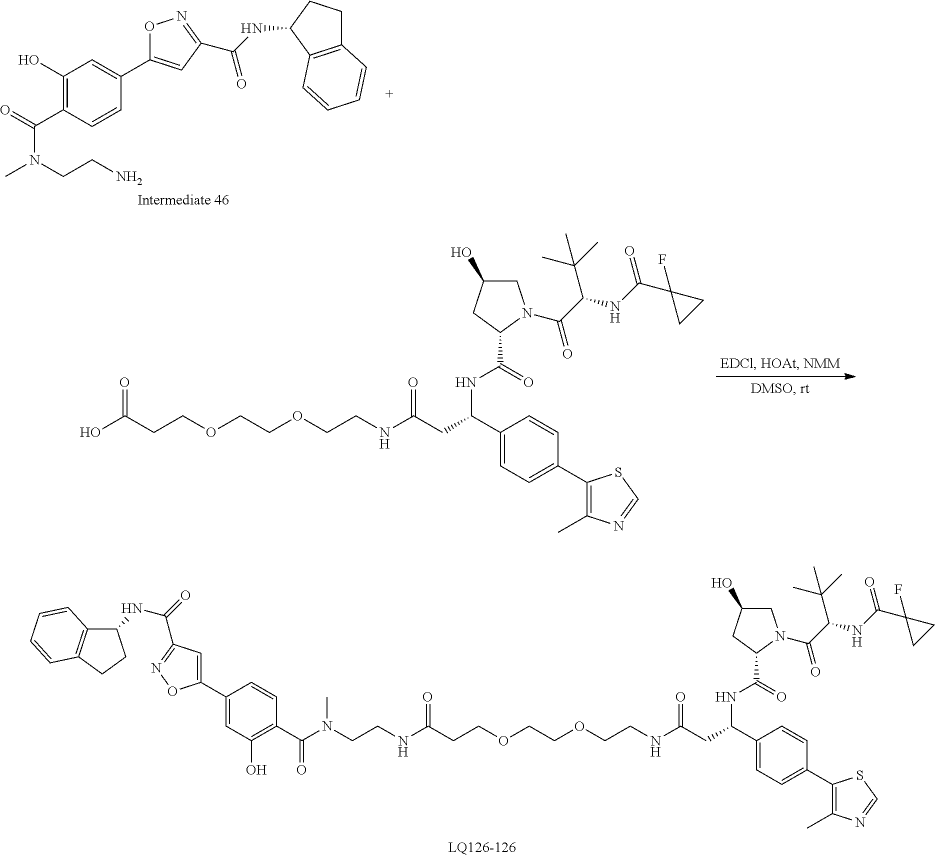 [SMILES: CN(CCN)C(=O)c1ccc(-c2cc(C(=O)N[C@@H]3CCc4ccccc43)no2)cc1O ; Cc1ncsc1-c1ccc([C@H](CC(=O)NCCOCCOCCC(=O)O)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCOCCOCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1]

LQ126-126 was synthesized following the standard procedure for preparing LQ126-89 from intermediate 46 (4 mg, 0.01 mmol), (S)-1-((2S,4R)-1-((S)-2-(1-fluorocyclopropane-1-carboxamido)-3,3-dimethylbutanoyl)-4-hydroxypyrrolidin-2-yl)-3-(4-(4-methylthiazol-5-yl)phenyl)-1,5-dioxo-9,12-dioxa-2,6-diazapentadecan-15-oic acid (7.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-126 was obtained as white solid (7 mg, 62%).

¹H NMR (600 MHz, Methanol-d₄) δ 8.99 (s, 1H), 7.50 (dd, J=9.3, 3.4 Hz, 1H), 7.48-7.41 (m, 4H), 7.39-7.20 (m, 6H), 7.15 (s, 1H), 5.66 (t, J=7.8 Hz, 1H), 5.33 (t, J=7.2 Hz, 1H), 4.75 (d, J=9.0 Hz, 1H), 4.63-4.57 (m, 1H), 4.47-4.44 (m, 1H), 3.84 (d, J=11.1 Hz, 1H), 3.80-3.38 (m, 12H), 3.18-3.07 (m, 3H), 3.02-2.91 (m, 4H), 2.90-2.83 (m, 1H), 2.81-2.72 (m, 1H), 2.66-2.57 (m, 1H), 2.49 (s, 3H), 2.41-2.38 (m, 1H), 2.21 (dd, J=13.3, 7.8 Hz, 1H), 2.12-2.03 (m, 1H), 2.00-1.92 (m, 1H), 1.42-1.27 (m, 6H), 1.07 (s, 9H). HRMS m/z [M+H]⁺ calcd for C₅₈H₇₁FN₉O₁₂S⁺ 1136.4921, found 1136.4917.

### Example 264

### Synthesis of LQ126-127

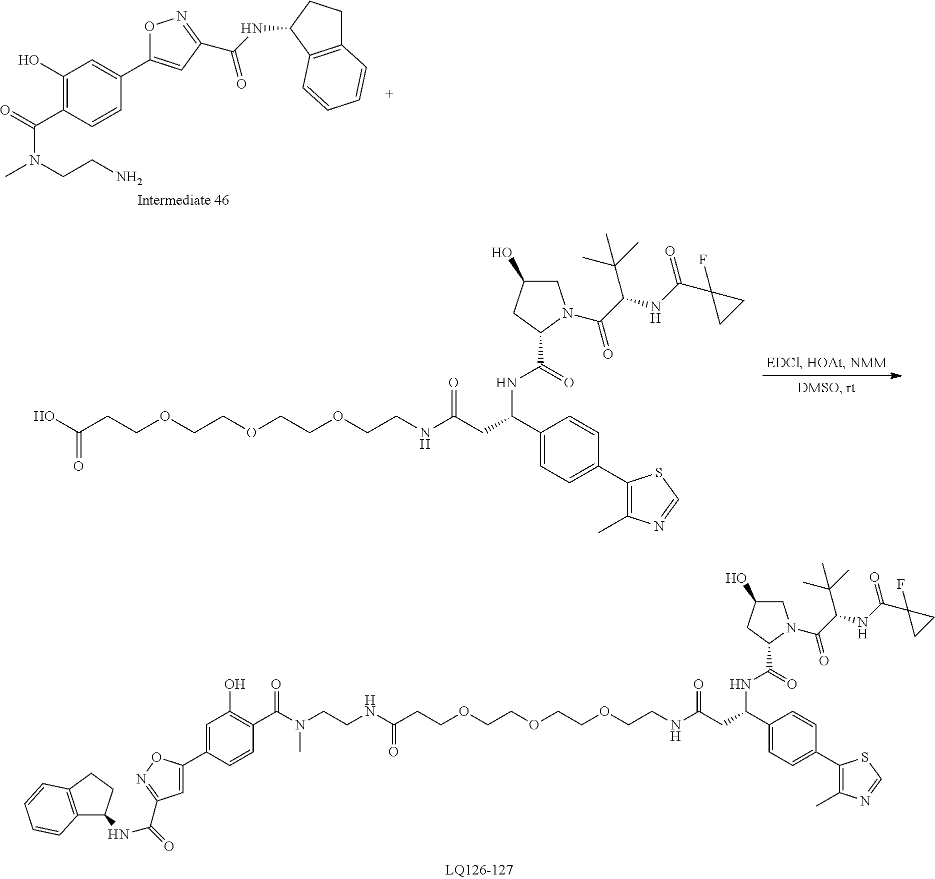 [SMILES: CN(CCN)C(=O)c1ccc(-c2cc(C(=O)N[C@@H]3CCc4ccccc43)no2)cc1O ; Cc1ncsc1-c1ccc([C@H](CC(=O)NCCOCCOCCOCCC(=O)O)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCOCCOCCOCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1]

LQ126-127 was synthesized following the standard procedure for preparing LQ126-89 from intermediate 46 (4 mg, 0.01 mmol), (S)-1-((2S,4R)-1-((S)-2-(1-fluorocyclopropane-1-carboxamido)-3,3-dimethylbutanoyl)-4-hydroxypyrrolidin-2-yl)-3-(4-(4-methylthiazol-5-yl)phenyl)-1,5-dioxo-9,12,15-trioxa-2,6-diazaoctadecan-18-oic acid (7.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-127 was obtained as white solid (8 mg, 68%). ¹H NMR (600 MHz, Methanol-d₄) δ 9.06 (s, 1H), 7.53-7.42 (m, 5H), 7.39-7.35 (m, 2H), 7.34-7.20 (m, 4H), 7.15 (s, 1H), 5.66 (t, J=7.8 Hz, 1H), 5.34 (t, J=7.1 Hz, 1H), 4.75 (d, J=9.1 Hz, 1H), 4.63-4.58 (m, 1H), 4.46 (s, 1H), 3.84 (d, J=11.1 Hz, 1H), 3.80-3.65 (m, 4H), 3.62-3.38 (m, 12H), 3.18-3.07 (m, 3H), 3.04-2.91 (m, 4H), 2.86 (dd, J=14.2, 6.2 Hz, 1H), 2.77 (dd, J=14.1, 8.1 Hz, 1H), 2.65-2.58 (m, 1H), 2.50 (s, 3H), 2.43-2.38 (m, 1H), 2.21 (dd, J=13.0, 7.7 Hz, 1H), 2.11-2.04 (m, 1H), 2.00-1.93 (m, 1H), 1.42-1.25 (m, 6H), 1.07 (s, 9H). HRMS m/z [M+H]⁺ calcd for C₆₀H₇₅FN₉O₁₃S⁺ 1180.5184, found 1180.5181.

### Example 265

### Synthesis of LQ126-128

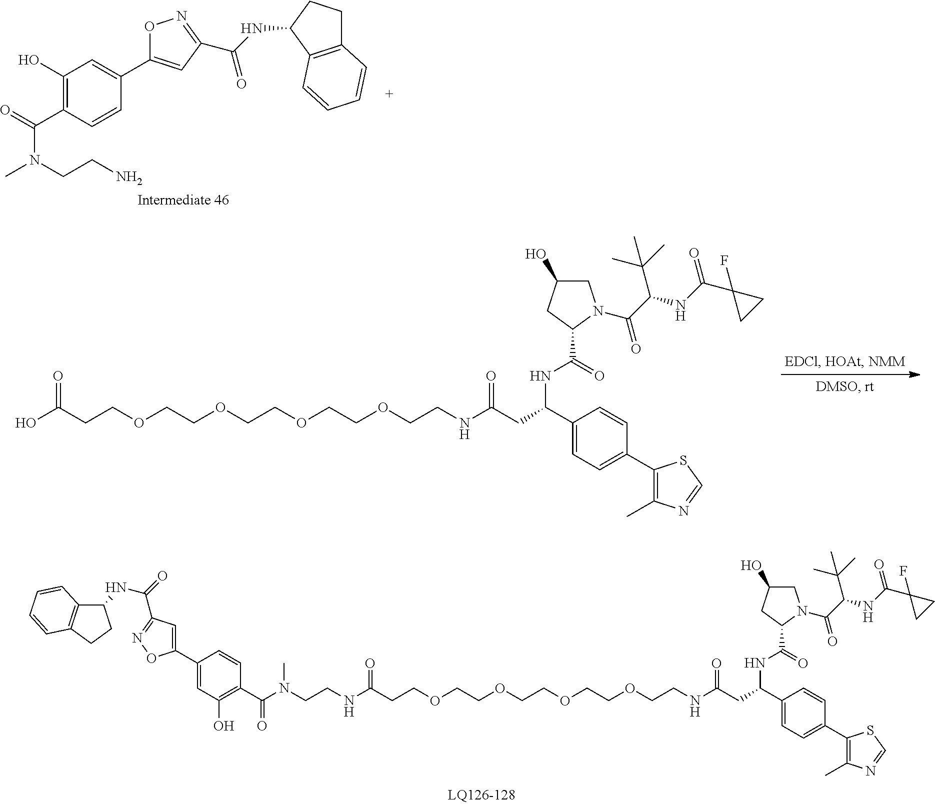 [SMILES: CN(CCN)C(=O)c1ccc(-c2cc(C(=O)N[C@@H]3CCc4ccccc43)no2)cc1O ; Cc1ncsc1-c1ccc([C@H](CC(=O)NCCOCCOCCOCCOCCC(=O)O)NC(=O)[C@@H]2C[C@@H](O)CN2C(=O)[C@@H](NC(=O)C2(F)CC2)C(C)(C)C)cc1 ; Cc1ncsc1-c1ccc([C@H](CC(=O)NCCOCCOCCOCCOCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1]

LQ126-128 was synthesized following the standard procedure for preparing LQ126-89 from intermediate 46 (4 mg, 0.01 mmol), (S)-1-((2S,4R)-1-((S)-2-(1-fluorocyclopropane-1-carboxamido)-3,3-dimethylbutanoyl)-4-hydroxypyrrolidin-2-yl)-3-(4-(4-methylthiazol-5-yl)phenyl)-1,5-dioxo-9,12,15,18-tetraoxa-2,6-diazahenicosan-21-oic acid (8.2 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-128 was obtained as white solid (7.5 mg, 61%). ¹H NMR (600 MHz, Methanol-d₄) δ 9.04 (s, 1H), 7.53-7.41 (m, 5H), 7.39-7.36 (m, 2H), 7.34-7.20 (m, 4H), 7.16 (s, 1H), 5.66 (t, J=7.7 Hz, 1H), 5.34 (t, J=7.1 Hz, 1H), 4.75 (d, J=9.2 Hz, 1H), 4.60 (t, J=8.5 Hz, 1H), 4.46 (s, 1H), 3.84 (d, J=11.1 Hz, 1H), 3.80-3.40 (m, 19H), 3.19-3.07 (m, 3H), 3.02-2.90 (m, 4H), 2.86 (dd, J=14.2, 6.1 Hz, 1H), 2.78 (dd, J=14.1, 8.1 Hz, 1H), 2.66-2.57 (m, 1H), 2.51 (s, 3H), 2.49-2.45 (m, 1H), 2.41-2.38 (m, 1H), 2.22 (dd, J=13.0, 7.8 Hz, 1H), 2.13-2.03 (m, 1H), 2.01-1.93 (m, 1H), 1.42-1.27 (m, 6H), 1.07 (s, 9H). HRMS m/z [M+H]⁺ calcd for C₆₂H₇₉FN₉O₁₄S⁺ 1224.5446, found 1224.5433.

### Example 266

### Synthesis of LQ126-130

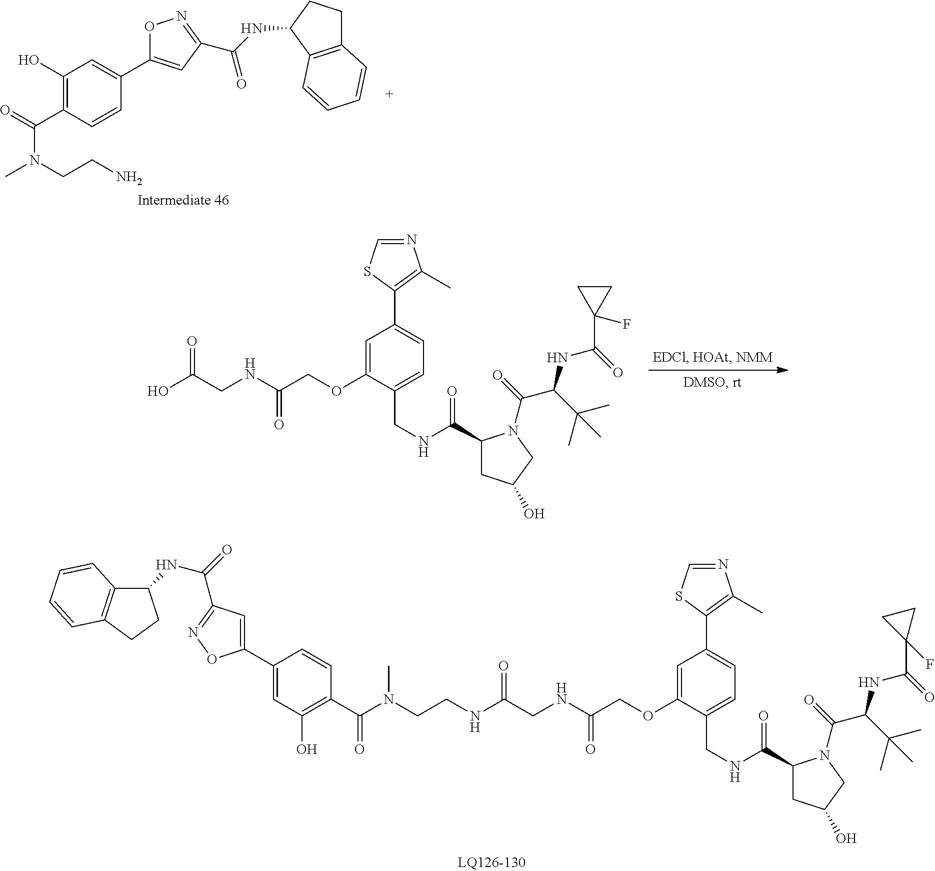 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCC(=O)O)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCC(=O)NCCN(C)C(=O)c2ccc(-c3cc(C(=O)N[C@@H]4CCc5ccccc54)no3)cc2O)c1]

LQ126-130 was synthesized following the standard procedure for preparing LQ126-89 from intermediate 46 (4 mg, 0.01 mmol), (2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetyl)glycine (6.5 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-130 was obtained as white solid (6 mg, 57%). ¹H NMR (600 MHz, Methanol-d₄) δ 9.01 (s, 1H), 7.54-7.43 (m, 2H), 7.42-7.20 (m, 5H), 7.14-7.00 (m, 4H), 5.70-5.61 (m, 1H), 4.78-4.55 (m, 5H), 4.50-4.42 (m, 2H), 4.06-3.89 (m, 2H), 3.87-3.67 (m, 3H), 3.61-3.39 (m, 1H), 3.17-3.07 (m, 3H), 3.02-2.90 (m, 4H), 2.65-2.57 (m, 1H), 2.49 (s, 3H), 2.21 (dd, J=13.3, 7.6 Hz, 1H), 2.14-2.03 (m, 2H), 1.41-1.23 (m, 4H), 1.00 (s, 9H). HRMS m/z [M+H]⁺ calcd for C₅₃H₆₁FN₉O₁₁S⁺ 1050.4190, found 1050.4214.

### Example 267

### Synthesis of LQ126-168

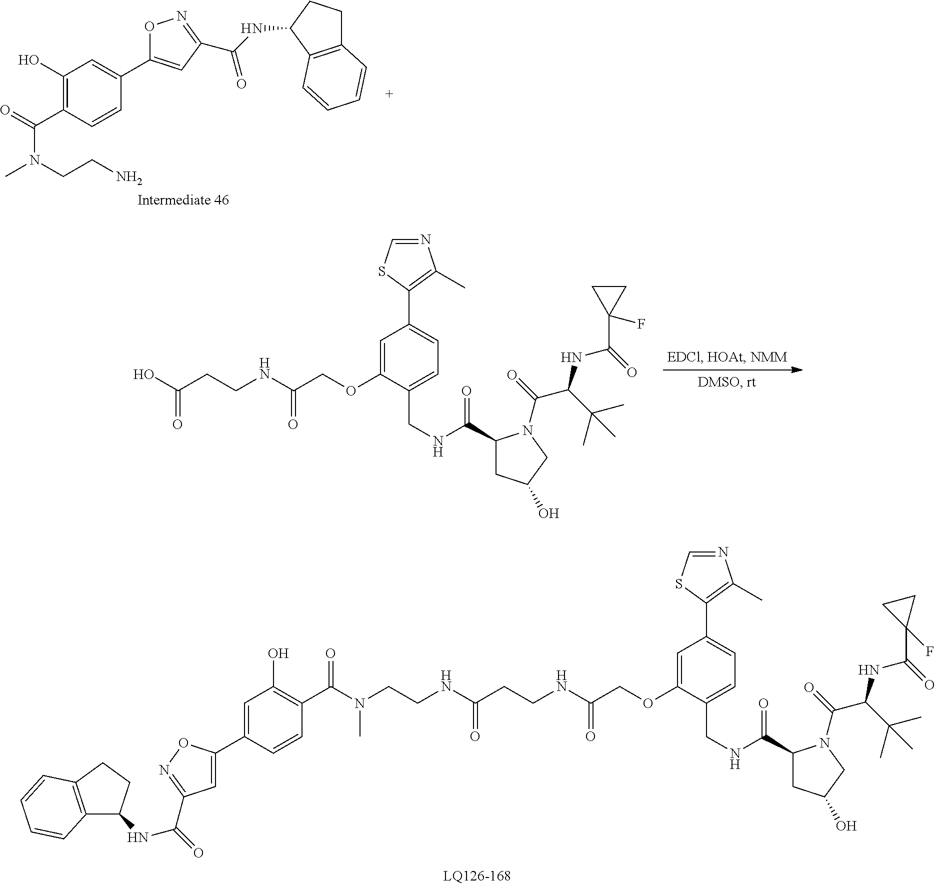 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCC(=O)O)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCC(=O)NCCN(C)C(=O)c2ccc(-c3cc(C(=O)N[C@@H]4CCc5ccccc54)no3)cc2O)c1]

LQ126-168 was synthesized following the standard procedure for preparing LQ126-89 from intermediate 46 (4 mg, 0.01 mmol), 3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetamido)propanoic acid (6.6 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-168 was obtained as white solid (6.9 mg, 65%). ¹H NMR (600 MHz, Methanol-d₄) δ 8.89 (s, 1H), 7.42-7.36 (m, 2H), 7.32-7.07 (m, 5H), 7.03-6.93 (m, 3H), 6.84 (s, 1H), 5.56-5.50 (m, 1H), 4.62 (d, J=9.2 Hz, 1H), 4.53-4.32 (m, 4H), 3.76-3.66 (m, 2H), 3.63-3.56 (m, 2H), 3.53-3.35 (m, 5H), 3.04-2.94 (m, 3H), 2.89-2.79 (m, 4H), 2.53-2.46 (m, 2H), 2.38 (s, 3H), 2.15-2.06 (m, 1H), 2.00-1.91 (m, 2H), 1.29-1.11 (m, 4H), 0.90 (s, 9H). HRMS m/z [M+H]⁺ calcd for C₅₄H₆₃FN₉O₁₁S⁺ 1064.4346, found 1064.4349.

### Example 268

### Synthesis of LQ126-170

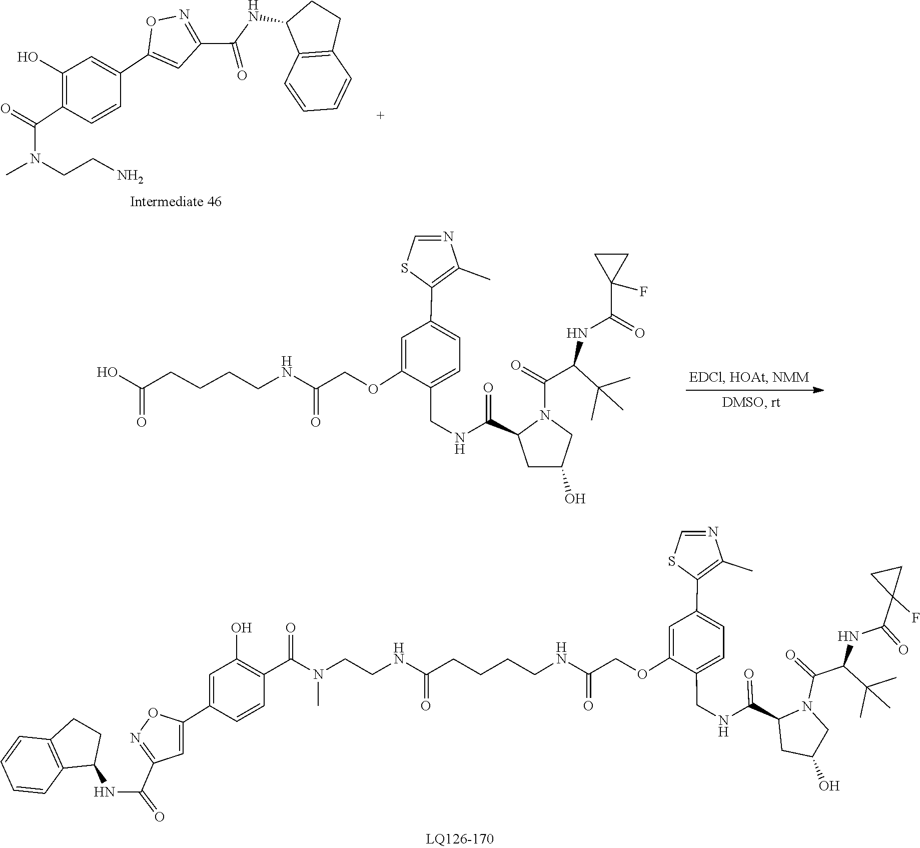 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCC(=O)O)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCC(=O)NCCN(C)C(=O)c2ccc(-c3cc(C(=O)N[C@@H]4CCc5ccccc54)no3)cc2O)c1]

LQ126-170 was synthesized following the standard procedure for preparing LQ126-89 from intermediate 46 (4 mg, 0.01 mmol), 5-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetamido)pentanoic acid (6.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-170 was obtained as white solid (7.6 mg, 70%). ¹H NMR (600 MHz, Methanol-d₄) δ 8.99 (s, 1H), 7.49 (d, J=7.7 Hz, 2H), 7.42 (s, 1H), 7.37-7.20 (m, 5H), 7.17-7.06 (m, 2H), 6.96 (s, 1H), 5.65 (t, J=7.7 Hz, 1H), 4.74 (d, J=9.2 Hz, 1H), 4.65-4.55 (m, 4H), 4.53-4.45 (m, 2H), 3.85 (d, J=11.1 Hz, 1H), 3.80 (dd, J=11.1, 3.8 Hz, 1H), 3.74-3.60 (m, 2H), 3.56-3.38 (m, 2H), 3.19-3.06 (m, 3H), 3.03-2.89 (m, 4H), 2.65-2.58 (m, 1H), 2.50 (s, 3H), 2.29-2.02 (m, 4H), 1.72-1.50 (m, 5H), 1.43-1.25 (m, 4H), 1.02 (s, 9H). HRMS m/z [M+H]⁺ calcd for C₅₆H₆₇FN₉O₁₁S⁺ 1092.4659, found 1092.4687.

### Example 269

### Synthesis of LQ126-171

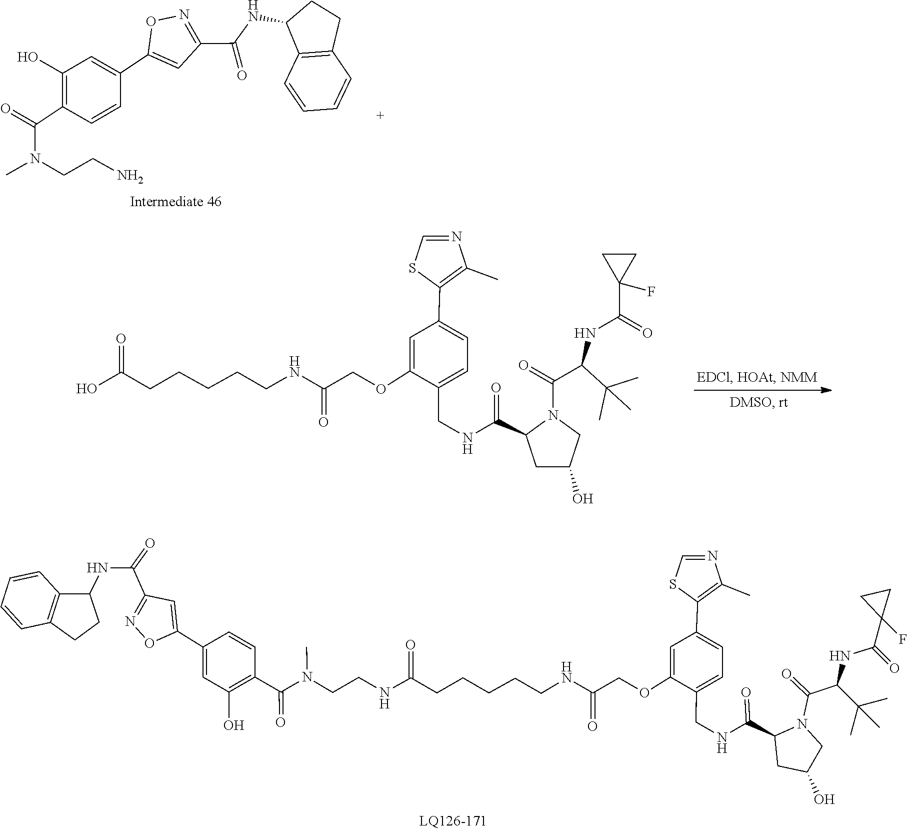 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCC(=O)O)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCC(=O)NCCN(C)C(=O)c2ccc(-c3cc(C(=O)NC4CCc5ccccc54)no3)cc2O)c1]

LQ126-171 was synthesized following the standard procedure for preparing LQ126-89 from intermediate 46 (4 mg, 0.01 mmol), 6-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetamido)hexanoic acid (7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-171 was obtained as white solid (6.9 mg, 63%). ¹H NMR (600 MHz, Methanol-d₄) δ 8.97 (s, 1H), 7.52-7.46 (m, 2H), 7.43 (dd, J=7.9, 1.6 Hz, 1H), 7.40-7.20 (m, 5H), 7.15 (s, 1H), 7.10 (dd, J=7.7, 1.6 Hz, 1H), 6.99-6.96 (m, 1H), 5.67-5.62 (m, 1H), 4.74 (d, J=9.3 Hz, 1H), 4.66-4.57 (m, 4H), 4.52-4.45 (m, 2H), 3.85 (d, J=11.1 Hz, 1H), 3.79 (dd, J=11.0, 3.9 Hz, 1H), 3.74-3.67 (m, 1H), 3.55-3.38 (m, 3H), 3.18-3.06 (m, 3H), 3.04-2.91 (m, 4H), 2.69-2.56 (m, 1H), 2.50 (s, 3H), 2.26-2.17 (m, 2H), 2.14-2.03 (m, 2H), 1.69-1.51 (m, 5H), 1.42-1.24 (m, 6H), 1.02 (s, 9H). HRMS m/z [M+H]⁺ calcd for C₅₇H₆₉FN₉O₁₁S⁺ 1106.4816, found 1106.4817.

### Example 270

### Synthesis of LQ126-172

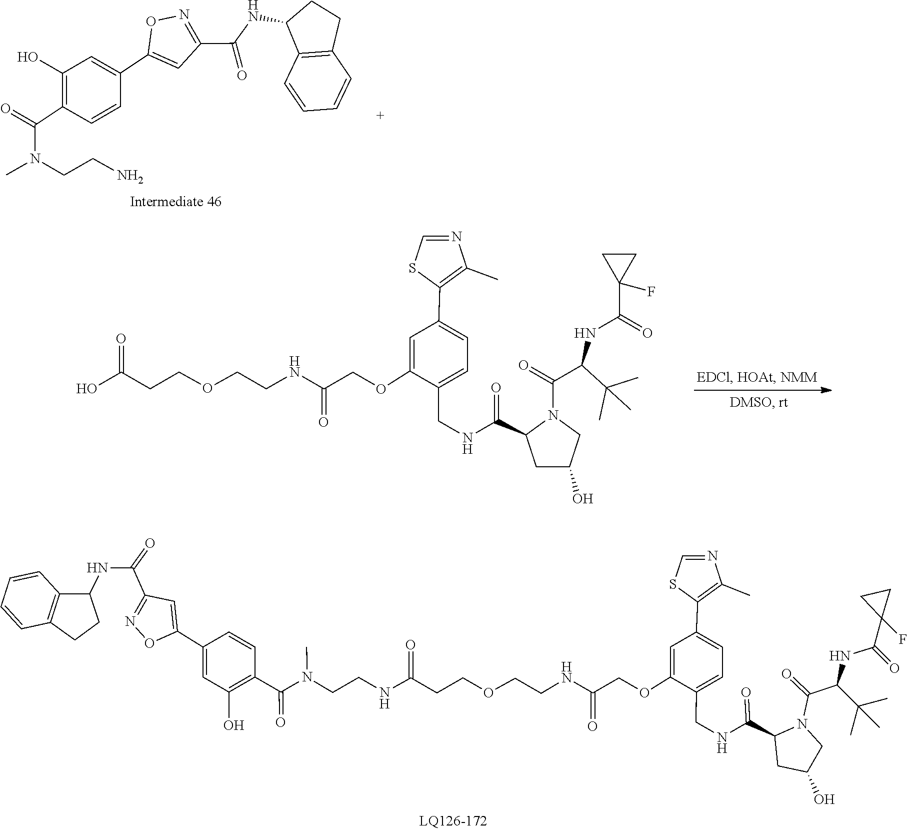 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCC(=O)O)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCC(=O)NCCN(C)C(=O)c2ccc(-c3cc(C(=O)NC4CCc5ccccc54)no3)cc2O)c1]

LQ126-172 was synthesized following the standard procedure for preparing LQ126-89 from intermediate 46 (4 mg, 0.01 mmol), 3-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetamido)ethoxy)propanoic acid (7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-172 was obtained as white solid (7.8 mg, 71%). ¹H NMR (600 MHz, Methanol-d₄) δ 8.94 (s, 1H), 7.49 (d, J=7.9 Hz, 2H), 7.42-7.20 (m, 6H), 7.12 (s, 1H), 7.09 (dd, J=7.7, 1.6 Hz, 1H), 6.97 (d, J=1.6 Hz, 1H), 5.65 (t, J=7.7 Hz, 1H), 4.75 (d, J=9.2 Hz, 1H), 4.66-4.59 (m, 2H), 4.56 (d, J=15.2 Hz, 1H), 4.52-4.46 (m, 2H), 3.86 (d, J=11.1 Hz, 1H), 3.80 (dd, J=11.2, 3.7 Hz, 1H), 3.76-3.63 (m, 2H), 3.61-3.43 (m, 6H), 3.16-3.07 (m, 3H), 3.00-2.91 (m, 4H), 2.64-2.58 (m, 1H), 2.49 (s, 3H), 2.47-2.33 (m, 2H), 2.26-2.19 (m, 1H), 2.13-2.03 (m, 1H), 1.41-1.25 (m, 6H), 1.03 (s, 9H). HRMS m/z [M+H]+ calcd for C₅₆H₆₇FN₉O₁₂S⁺ 1108.4608, found 1108.4611.

### Example 271

### Synthesis of LQ126-173

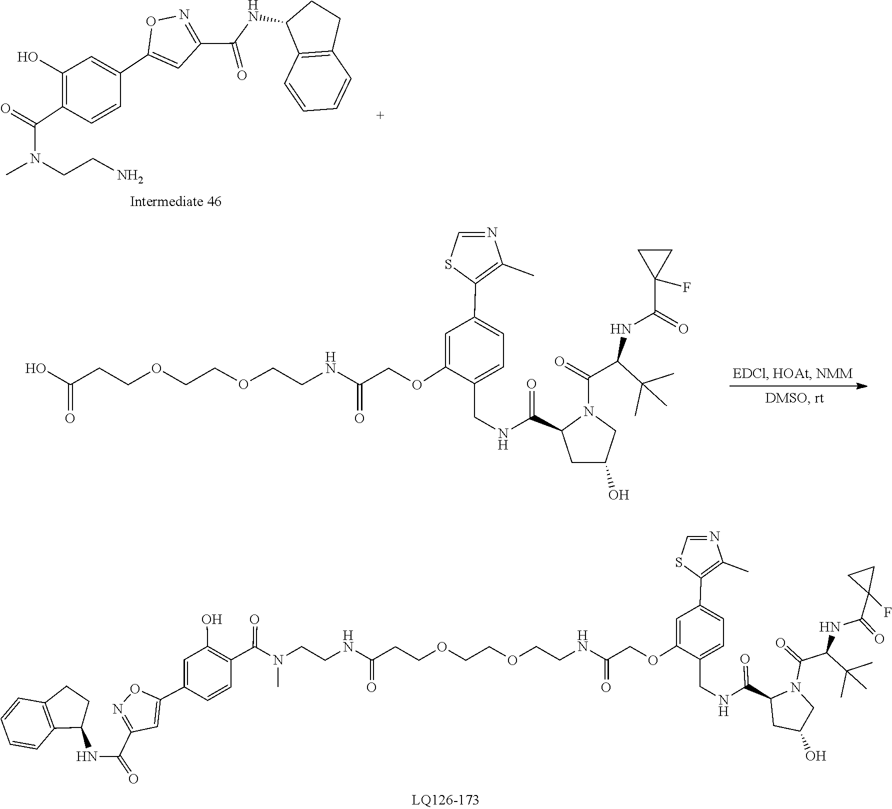 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCOCCC(=O)O)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCOCCC(=O)NCCN(C)C(=O)c2ccc(-c3cc(C(=O)N[C@@H]4CCc5ccccc54)no3)cc2O)c1]

LQ126-173 was synthesized following the standard procedure for preparing LQ126-89 from intermediate 46 (4 mg, 0.01 mmol), 3-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetamido)ethoxy)ethoxy)propanoic acid (7.5 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-173 was obtained as white solid (7.8 mg, 68%). ¹H NMR (600 MHz, Methanol-d₄) δ 9.00 (s, 1H), 7.53-7.49 (m, 1H), 7.42 (dd, J=7.9, 1.5 Hz, 1H), 7.37-7.34 (m, 2H), 7.32 (d, J=7.3 Hz, 1H), 7.29 (d, J=7.3 Hz, 1H), 7.27-7.20 (m, 2H), 7.14 (s, 1H), 7.09 (dd, J=7.7, 1.6 Hz, 1H), 6.98 (d, J=1.6 Hz, 1H), 5.65 (t, J=7.7 Hz, 1H), 4.75 (d, J=9.1 Hz, 1H), 4.67-4.58 (m, 3H), 4.56-4.47 (m, 2H), 3.85 (d, J=11.1 Hz, 1H), 3.81 (dd, J=11.1, 3.7 Hz, 1H), 3.75-3.63 (m, 2H), 3.62-3.38 (m, 9H), 3.15-3.06 (m, 3H), 3.02-2.90 (m, 4H), 2.65-2.57 (m, 1H), 2.50 (s, 3H), 2.49-2.42 (m, 1H), 2.41-2.34 (m, 1H), 2.26-2.20 (m, 1H), 2.13-2.03 (m, 2H), 1.40-1.24 (m, 6H), 1.03 (s, 9H). HRMS m/z [M+H]⁺ calcd for C₅₈H₇₁FN₉O₁₃S⁺ 1152.4871, found 1152.4870.

### Example 272

### Synthesis of LQ126-174

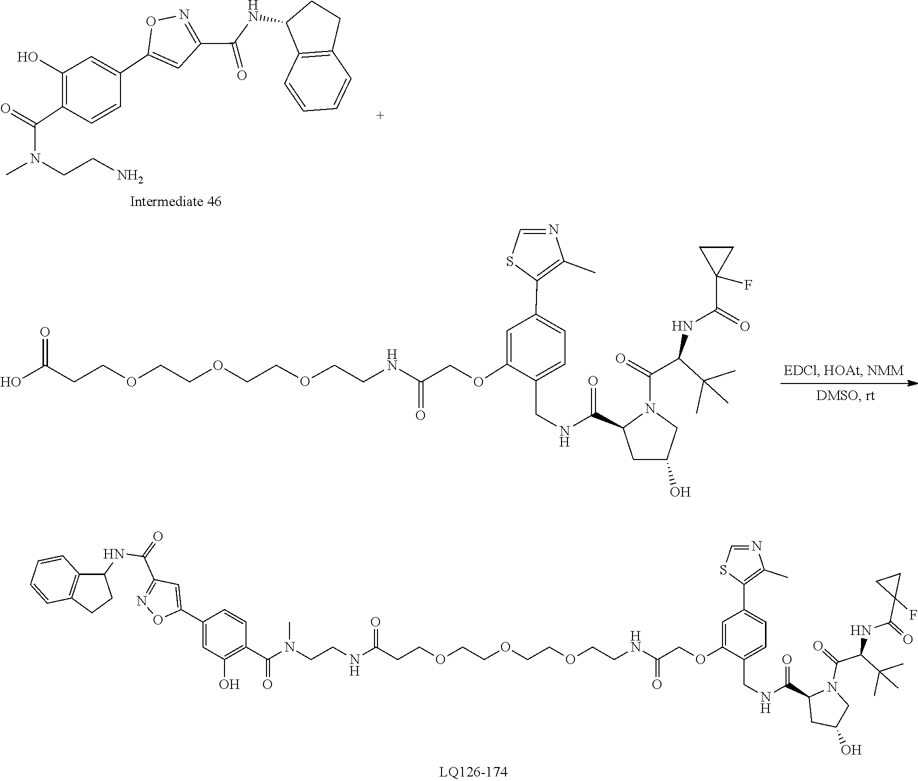 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCOCCOCCC(=O)O)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCOCCOCCC(=O)NCCN(C)C(=O)c2ccc(-c3cc(C(=O)NC4CCc5ccccc54)no3)cc2O)c1]

LQ126-174 was synthesized following the standard procedure for preparing LQ126-89 from intermediate 46 (4 mg, 0.01 mmol), 1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-2-oxo-6,9,12-trioxa-3-azapentadecan-15-oic acid (7.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-174 was obtained as white solid (8.2 mg, 69%). ¹H NMR (600 MHz, Methanol-d₄) δ 9.01 (s, 1H), 7.51 (t, J=8.0 Hz, 1H), 7.43 (dd, J=7.8, 1.5 Hz, 1H), 7.38-7.34 (m, 2H), 7.32 (d, J=7.3 Hz, 1H), 7.29 (d, J=7.3 Hz, 1H), 7.27-7.20 (m, 2H), 7.15 (s, 1H), 7.10 (dd, J=7.7, 1.6 Hz, 1H), 6.99 (d, J=1.6 Hz, 1H), 5.65 (t, J=7.8 Hz, 1H), 4.75 (d, J=9.1 Hz, 1H), 4.67-4.59 (m, 3H), 4.56 (d, J=15.2 Hz, 1H), 4.53-4.48 (m, 1H), 3.85 (d, J=11.1 Hz, 1H), 3.81 (dd, J=11.0, 3.8 Hz, 1H), 3.75-3.64 (m, 2H), 3.62-3.52 (m, 11H), 3.49 (t, J=5.6 Hz, 2H), 3.17-3.06 (m, 3H), 3.03-2.91 (m, 4H), 2.64-2.57 (m, 1H), 2.51 (s, 3H), 2.49-2.37 (m, 2H), 2.23 (dd, J=13.1, 7.8 Hz, 1H), 2.13-2.03 (m, 2H), 1.41-1.26 (m, 6H), 1.03 (s, 9H). HRMS m/z [M+H]⁺ calcd for C₆₀H₇₅FN₉O₁₄S⁺ 1196.5133, found 1196.5130.

### Example 273

### Synthesis of LQ126-175

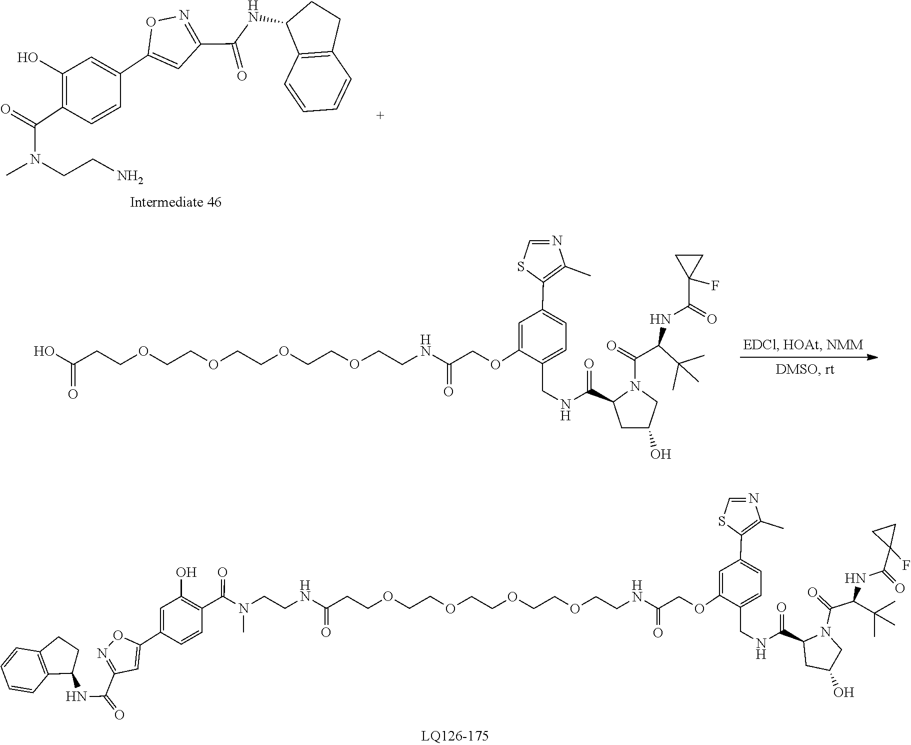 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCOCCOCCOCCC(=O)O)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCOCCOCCOCCC(=O)NCCN(C)C(=O)c2ccc(-c3cc(C(=O)N[C@@H]4CCc5ccccc54)no3)cc2O)c1]

LQ126-175 was synthesized following the standard procedure for preparing LQ126-89 from intermediate 46 (4 mg, 0.01 mmol), 1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-2-oxo-6,9,12,15-tetraoxa-3-azaoctadecan-18-oic acid (8.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-175 was obtained as white solid (7.9 mg, 64%). ¹H NMR (600 MHz, Methanol-d₄) δ 8.97 (s, 1H), 7.53-7.49 (m, 2H), 7.45-7.42 (m, 1H), 7.39-7.34 (m, 1H), 7.32 (d, J=7.3 Hz, 1H), 7.30-7.20 (m, 3H), 7.15 (s, 1H), 7.10 (d, J=7.7 Hz, 2H), 6.99 (d, J=1.5 Hz, 1H), 5.65 (t, J=7.7 Hz, 1H), 4.75 (d, J=9.2 Hz, 1H), 4.67-4.54 (m, 4H), 4.50 (d, J=15.0 Hz, 1H), 3.85 (d, J=11.1 Hz, 1H), 3.81 (dd, J=11.0, 3.8 Hz, 1H), 3.75-3.64 (m, 2H), 3.63-3.52 (m, 15H), 3.51-3.46 (m, 2H), 3.22-3.20 (m, 1H), 3.17-3.06 (m, 3H), 3.03-2.91 (m, 4H), 2.64-2.57 (m, 1H), 2.50 (s, 3H), 2.49-2.36 (m, 2H), 2.25-2.20 (m, 1H), 2.13-2.03 (m, 2H), 1.41-1.26 (m, 6H), 1.03 (s, 9H). HRMS m/z [M+H]+ calcd for C₆₂H₇₉FN₉O₁₅S⁺ 1240.5395, found 1240.5398.

### Example 274

### Synthesis of LQ126-176

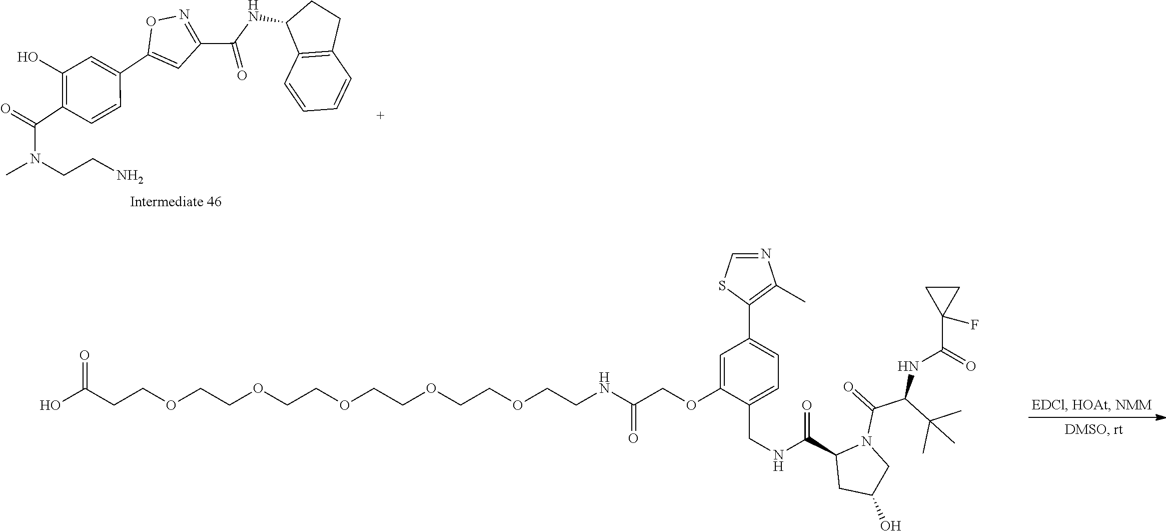 [SMILES: CN(CCN)C(=O)c1ccc(-c2cc(C(=O)N[C@@H]3CCc4ccccc43)no2)cc1O ; Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCOCCOCCOCCOCCC(=O)O)c1]

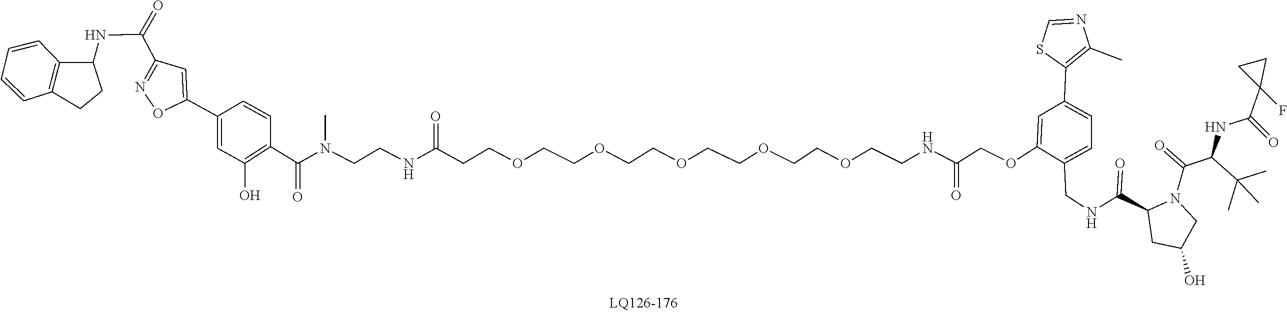 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCOCCOCCOCCOCCC(=O)NCCN(C)C(=O)c2ccc(-c3cc(C(=O)NC4CCc5ccccc54)no3)cc2O)c1]

LQ126-176 was synthesized following the standard procedure for preparing LQ126-89 from intermediate 46 (4 mg, 0.01 mmol), 1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-2-oxo-6,9,12,15,18-pentaoxa-3-azahenicosan-21-oic acid (8.8 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-176 was obtained as white solid (7.7 mg, 60%). ¹H NMR (600 MHz, Methanol-d₄) δ 8.94 (s, 1H), 7.52-7.49 (m, 2H), 7.45-7.42 (m, 1H), 7.38-7.35 (m, 1H), 7.32 (d, J=7.2 Hz, 1H), 7.29 (d, J=7.4 Hz, 1H), 7.27-7.20 (m, 2H), 7.16 (s, 1H), 7.11-7.08 (m, 1H), 6.99 (d, J=1.6 Hz, 1H), 5.65 (t, J=7.7 Hz, 1H), 4.75 (d, J=9.4 Hz, 1H), 4.66-4.64 (m, 2H), 4.63-4.55 (m, 2H), 4.53-4.48 (m, 1H), 3.85 (d, J=11.2 Hz, 1H), 3.81 (dd, J=11.0, 3.8 Hz, 1H), 3.63-3.55 (m, 20H), 3.52-3.48 (m, 2H), 3.22-3.20 (m, 1H), 3.17-3.07 (m, 3H), 3.03-2.91 (m, 4H), 2.64-2.58 (m, 1H), 2.50 (s, 3H), 2.49-2.37 (m, 2H), 2.26-2.19 (m, 1H), 2.12-2.04 (m, 2H), 1.42-1.25 (m, 6H), 1.03 (s, 9H). HRMS m/z [M+H]⁺ calcd for C₆₄H₈₃FN₉O₁₆S⁺ 1284.5657, found 1284.5653.

### Example 275

### Synthesis of Intermediate 47

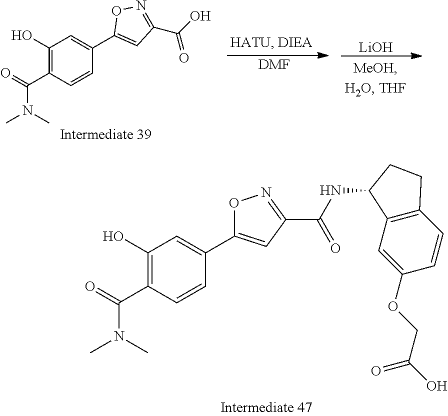

Intermediate 47 was synthesized according to the procedures for the preparation of intermediate 4 as a white solid in 58% yield. ¹H NMR (600 MHz, Methanol-d₄) δ 7.44 (dd, J=7.9, 1.5 Hz, 1H), 7.37 (d, J=1.5 Hz, 1H), 7.35 (d, J=7.9 Hz, 1H), 7.19 (d, J=8.3 Hz, 1H), 7.15 (s, 1H), 6.91 (d, J=2.5 Hz, 1H), 6.86 (dd, J=8.3, 2.5 Hz, 1H), 5.62 (t, J=7.7 Hz, 1H), 4.64 (s, 2H), 3.20-2.93 (m, 7H), 2.91-2.83 (m, 1H), 2.66-2.58 (m, 1H), 2.12-2.03 (m, 1H). MS (ESI): m/z 466.5 [M+H]⁺.

### Example 276

### Synthesis of LQ126-177

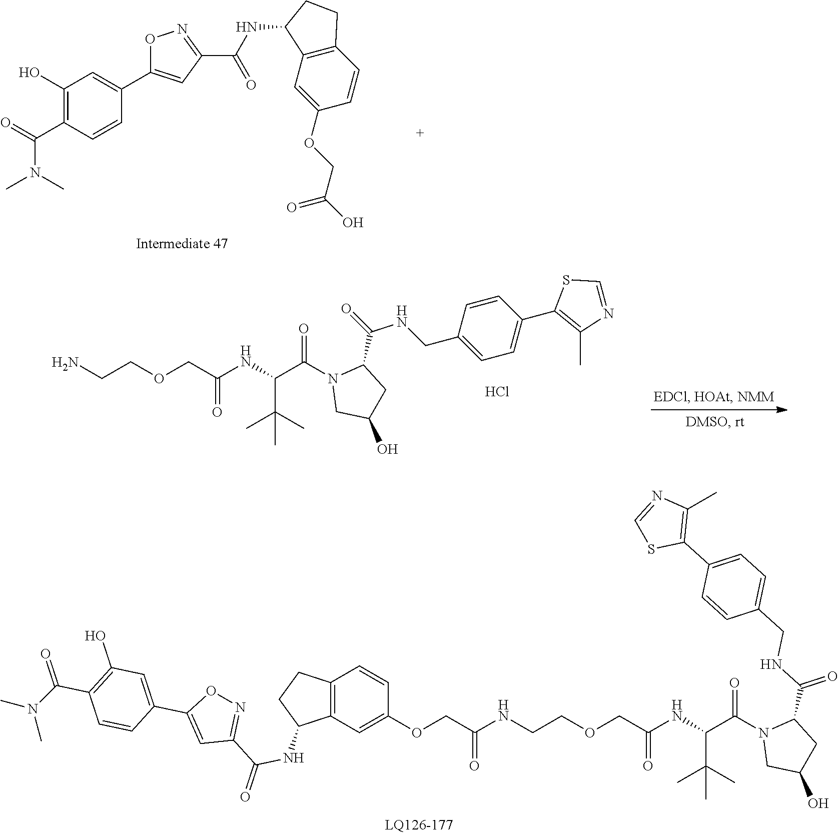 [SMILES: CN(C)C(=O)c1ccc(-c2cc(C(=O)N[C@@H]3CCc4ccc(OCC(=O)O)cc43)no2)cc1O ; Cc1ncsc1-c1ccc(CNC(=O)[C@@H]2C[C@@H](O)CN2C(=O)[C@@H](NC(=O)COCCN)C(C)(C)C)cc1 ; Cl ; Cc1ncsc1-c1ccc(CNC(=O)[C@@H]2C[C@@H](O)CN2C(=O)[C@@H](NC(=O)COCCNC(=O)COc2ccc3c(c2)[C@H](NC(=O)c2cc(-c4ccc(C(=O)N(C)C)c(O)c4)on2)CC3)C(C)(C)C)cc1]

To a solution of Intermediate 47 (5 mg, 0.01 mmol) in DMSO (1 mL) were added (2S,4R)-1-((S)-2-(2-(2-aminoethoxy)acetamido)-3,3-dimethylbutanoyl)-4-hydroxy-N-(4-(4-methylthiazol-5-yl)benzyl)pyrrolidine-2-carboxamide (5.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv). After being stirred overnight at room temperature, the resulting mixture was purified by preparative HPLC (5%-70% acetonitrile/0.1% TFA in H₂O) to afford LQ126-177 as white solid (7.2 mg, 74%). ¹H NMR (600 MHz, Methanol-d₄) δ 9.10 (s, 1H), 7.58-7.29 (m, 7H), 7.15-7.11 (m, 2H), 6.94-6.84 (m, 2H), 5.54 (t, J=7.8 Hz, 1H), 4.72-4.67 (m, 1H), 4.62 (t, J=8.4 Hz, 1H), 4.58-4.47 (m, 5H), 4.02 (dd, J=15.2, 1.4 Hz, 1H), 3.92-3.84 (m, 2H), 3.79 (dd, J=11.0, 3.7 Hz, 1H), 3.69-3.54 (m, 2H), 3.53-3.45 (m, 2H), 3.18-2.90 (m, 7H), 2.84-2.74 (m, 1H), 2.59-2.50 (m, 1H), 2.46 (s, 3H), 2.28-2.21 (m, 1H), 2.13-2.00 (m, 2H), 1.01 (s, 9H). HRMS m/z [M+H]⁺ calcd for C₅₀H₅₉N₈O₁₁S⁺ 979.4019, found 979.4016.

### Example 277

### Synthesis of LQ126-178

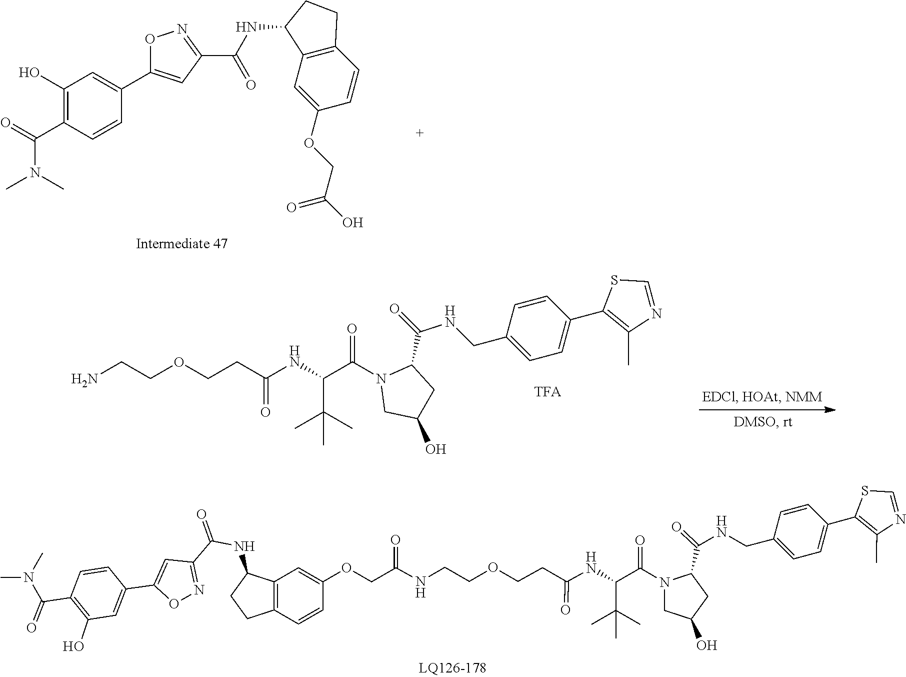 [SMILES: CN(C)C(=O)c1ccc(-c2cc(C(=O)N[C@@H]3CCc4ccc(OCC(=O)O)cc43)no2)cc1O ; Cc1ncsc1-c1ccc(CNC(=O)[C@@H]2C[C@@H](O)CN2C(=O)[C@@H](NC(=O)CCOCCN)C(C)(C)C)cc1 ; Cc1ncsc1-c1ccc(CNC(=O)[C@@H]2C[C@@H](O)CN2C(=O)[C@@H](NC(=O)CCOCCNC(=O)COc2ccc3c(c2)[C@H](NC(=O)c2cc(-c4ccc(C(=O)N(C)C)c(O)c4)on2)CC3)C(C)(C)C)cc1 ; "TFA"]

LQ126-178 was synthesized following the standard procedure for preparing LQ126-177 from intermediate 47 (5 mg, 0.01 mmol), (2S,4R)-1-((S)-2-(3-(2-aminoethoxy)propanamido)-3,3-dimethylbutanoyl)-4-hydroxy-N-(4-(4-methylthiazol-5-yl)benzyl)pyrrolidine-2-carboxamide (6.4 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-178 was obtained as white solid (6.1 mg, 62%). ¹H NMR (600 MHz, Methanol-d₄) δ 9.19 (s, 1H), 7.50-7.29 (m, 7H), 7.23-7.13 (m, 2H), 6.98-6.84 (m, 2H), 5.56 (t, J=8.0 Hz, 1H), 4.68-4.60 (m, 2H), 4.54-4.41 (m, 4H), 4.33 (d, J=15.5 Hz, 1H), 3.86 (d, J=11.1 Hz, 1H), 3.73 (dd, J=11.0, 3.9 Hz, 1H), 3.69-3.62 (m, 1H), 3.60-3.51 (m, 2H), 3.50-3.39 (m, 3H), 3.21-2.89 (m, 7H), 2.87-2.79 (m, 1H), 2.61-2.53 (m, 1H), 2.49 (s, 3H), 2.44-2.33 (m, 1H), 2.27-2.20 (m, 1H), 2.11-2.01 (m, 3H), 1.01 (s, 9H). HRMS m/z [M+H]⁺ calcd for C₅₁H₆₁N₈O₁₁S⁺ 993.4175, found 993.4179.

### Example 278

### Synthesis of LQ126-180

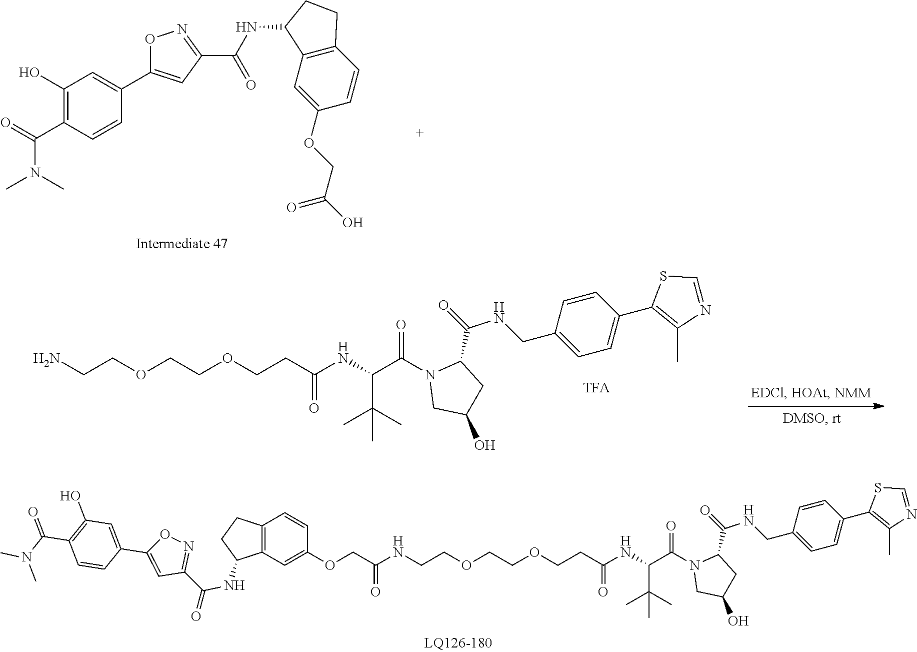

LQ126-180 was synthesized following the standard procedure for preparing LQ126-177 from intermediate 47 (5 mg, 0.01 mmol), (2S,4R)-1-((S)-2-(3-(2-(2-aminoethoxy)ethoxy)propanamido)-3,3-dimethylbutanoyl)-4-hydroxy-N-(4-(4-methylthiazol-5-yl)benzyl)pyrrolidine-2-carboxamide (6.8 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-180 was obtained as white solid (7.1 mg, 69%). ¹H NMR (600 MHz, Methanol-d₄) δ 9.19 (s, 1H), 7.50-7.45 (m, 2H), 7.43-7.40 (m, 3H), 7.37-7.32 (m, 2H), 7.20-7.15 (m, 2H), 6.93 (d, J=2.5 Hz, 1H), 6.87 (dd, J=8.2, 2.5 Hz, 1H), 5.59 (t, J=7.8 Hz, 1H), 4.65 (s, 1H), 4.61-4.51 (m, 2H), 4.51-4.42 (m, 3H), 4.34 (d, J=15.5 Hz, 1H), 3.86 (d, J=11.0 Hz, 1H), 3.76 (dd, J=10.9, 3.9 Hz, 1H), 3.74-3.62 (m, 2H), 3.60-3.50 (m, 6H), 3.42 (t, J=5.5 Hz, 2H), 3.18-2.92 (m, 7H), 2.89-2.80 (m, 1H), 2.62-2.55 (m, 1H), 2.52-2.39 (m, 5H), 2.24-2.17 (m, 1H), 2.12-2.02 (m, 2H), 1.01 (s, 9H). HRMS m/z [M+H]⁺ calcd for C₅₃H₆₅N₈O₁₂S⁺ 1037.4437, found 1037.4443.

### Example 279

### Synthesis of LQ126-181

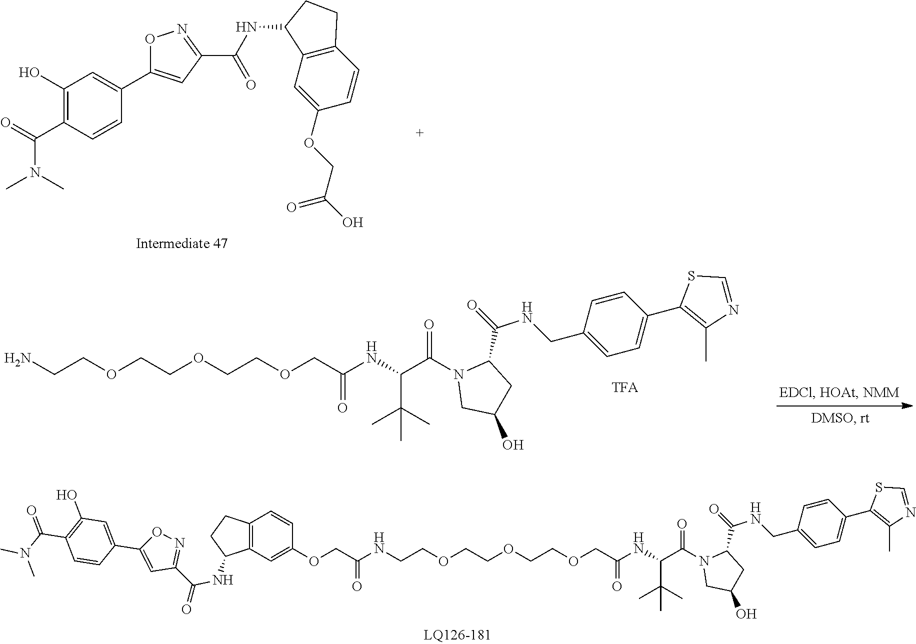

LQ126-181 was synthesized following the standard procedure for preparing LQ126-177 from intermediate 47 (5 mg, 0.01 mmol), (2S,4R)-1-((S)-14-amino-2-(tert-butyl)-4-oxo-6,9,12-trioxa-3-azatetradecanoyl)-4-hydroxy-N-(4-(4-methylthiazol-5-yl)benzyl)pyrrolidine-2-carboxamide (7.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-181 was obtained as white solid (7 mg, 66%). ¹H NMR (600 MHz, Methanol-d₄) δ 9.10 (s, 1H), 7.56-7.32 (m, 7H), 7.25-7.14 (m, 2H), 6.98-6.85 (m, 2H), 5.64-5.56 (m, 1H), 4.72 (s, 1H), 4.64-4.29 (m, 6H), 4.05-3.71 (m, 4H), 3.71-3.50 (m, 10H), 3.47-3.41 (m, 2H), 3.24-2.82 (m, 8H), 2.64-2.56 (m, 1H), 2.49 (s, 3H), 2.27-2.21 (m, 1H), 2.17-2.02 (m, 2H), 1.03 (s, 9H). HRMS m/z [M+H]⁺ calcd for C₅₄H₆₇N₈O₁₃S⁺ 1067.4543, found 1067.4537.

### Example 280

### Synthesis of LQ126-182

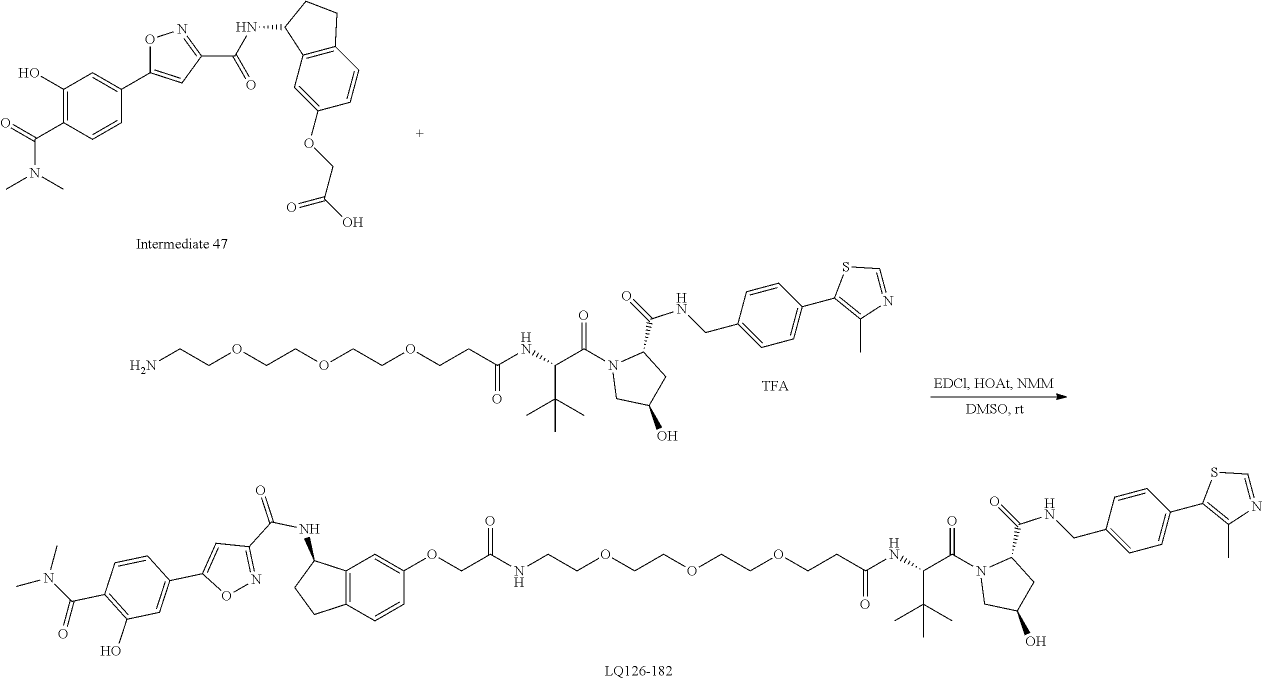

LQ126-182 was synthesized following the standard procedure for preparing LQ126-177 from intermediate 47 (5 mg, 0.01 mmol), (2S,4R)-1-((S)-1-amino-14-(tert-butyl)-12-oxo-3,6,9-trioxa-13-azapentadecan-15-oyl)-4-hydroxy-N-(4-(4-methylthiazol-5-yl)benzyl)pyrrolidine-2-carboxamide (7.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-182 was obtained as white solid (6.5 mg, 60%). ¹H NMR (600 MHz, Methanol-d₄) δ 9.14 (s, 1H), 7.57-7.29 (m, 7H), 7.25-7.13 (m, 2H), 7.03-6.84 (m, 2H), 5.69-5.56 (m, 1H), 4.68-4.45 (m, 6H), 4.36 (d, J=15.0 Hz, 1H), 3.89 (d, J=10.9 Hz, 1H), 3.83-3.77 (m, 1H), 3.75-3.51 (m, 12H), 3.47-3.42 (m, 2H), 3.19-2.83 (m, 8H), 2.64-2.41 (m, 6H), 2.26-2.19 (m, 1H), 2.12-2.03 (m, 2H), 1.03 (s, 9H). HRMS m/z [M+H]⁺ calcd for C₅₅H₆₉N₈O₁₃S⁺ 1081.4699, found 1081.4670.

### Example 281

### Synthesis of LQ126-183

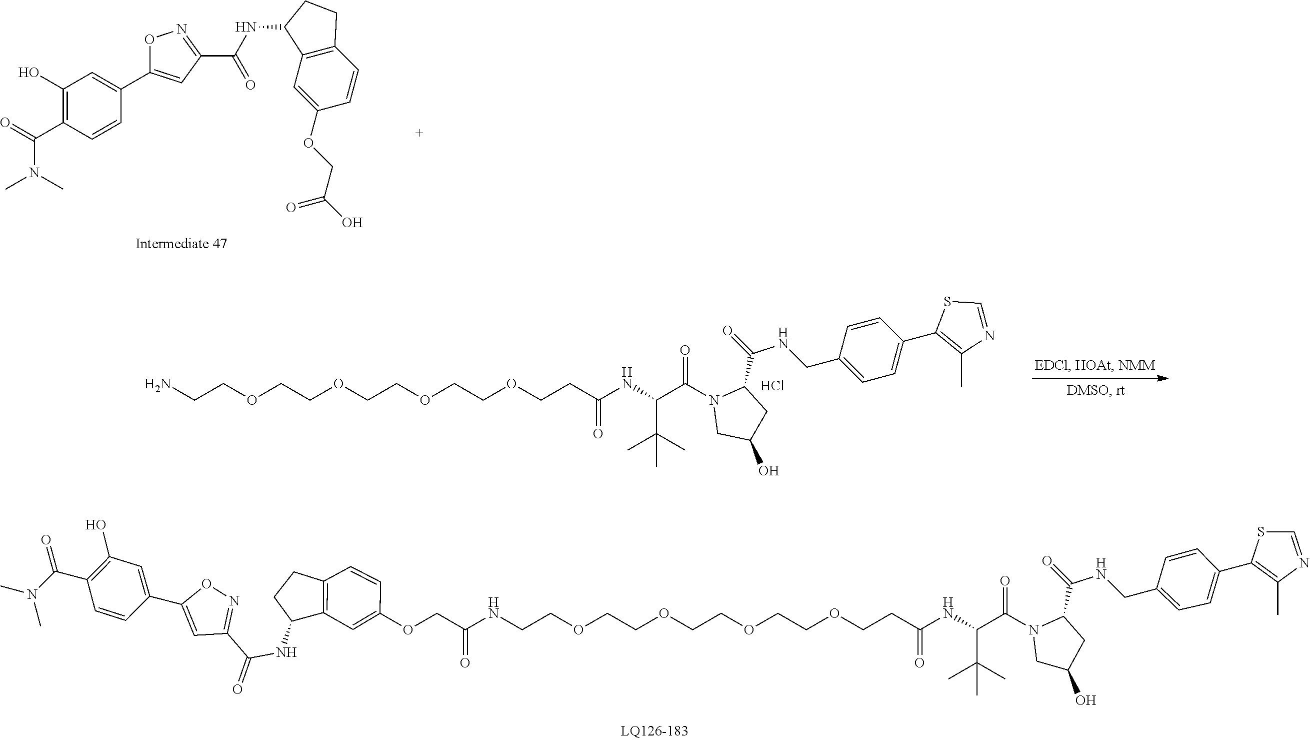

LQ126-183 was synthesized following the standard procedure for preparing LQ126-177 from intermediate 47 (5 mg, 0.01 mmol), (2S,4R)-1-((S)-1-amino-17-(tert-butyl)-15-oxo-3,6,9,12-tetraoxa-16-azaoctadecan-18-oyl)-4-hydroxy-N-(4-(4-methylthiazol-5-yl)benzyl)pyrrolidine-2-carboxamide (7.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-183 was obtained as white solid (6.4 mg, 57%). ¹H NMR (600 MHz, Methanol-d₄) δ 9.27 (s, 1H), 7.59-7.32 (m, 7H), 7.24-7.13 (m, 2H), 7.05-6.88 (m, 2H), 5.75-5.54 (m, 1H), 4.72-4.45 (m, 6H), 4.37 (d, J=15.3 Hz, 1H), 3.90 (d, J=11.1 Hz, 1H), 3.83-3.78 (m, 1H), 3.73-3.49 (m, 16H), 3.49-3.41 (m, 2H), 3.20-2.81 (m, 8H), 2.67-2.42 (m, 6H), 2.29-2.20 (m, 1H), 2.14-2.04 (m, 2H), 1.03 (s, 9H). HRMS m/z [M+H]⁺ calcd for C₅₇H₇₃N₈O₁₄S⁺ 1125.4961, found 1125.4937.

### Example 282

### Synthesis of LQ126-184

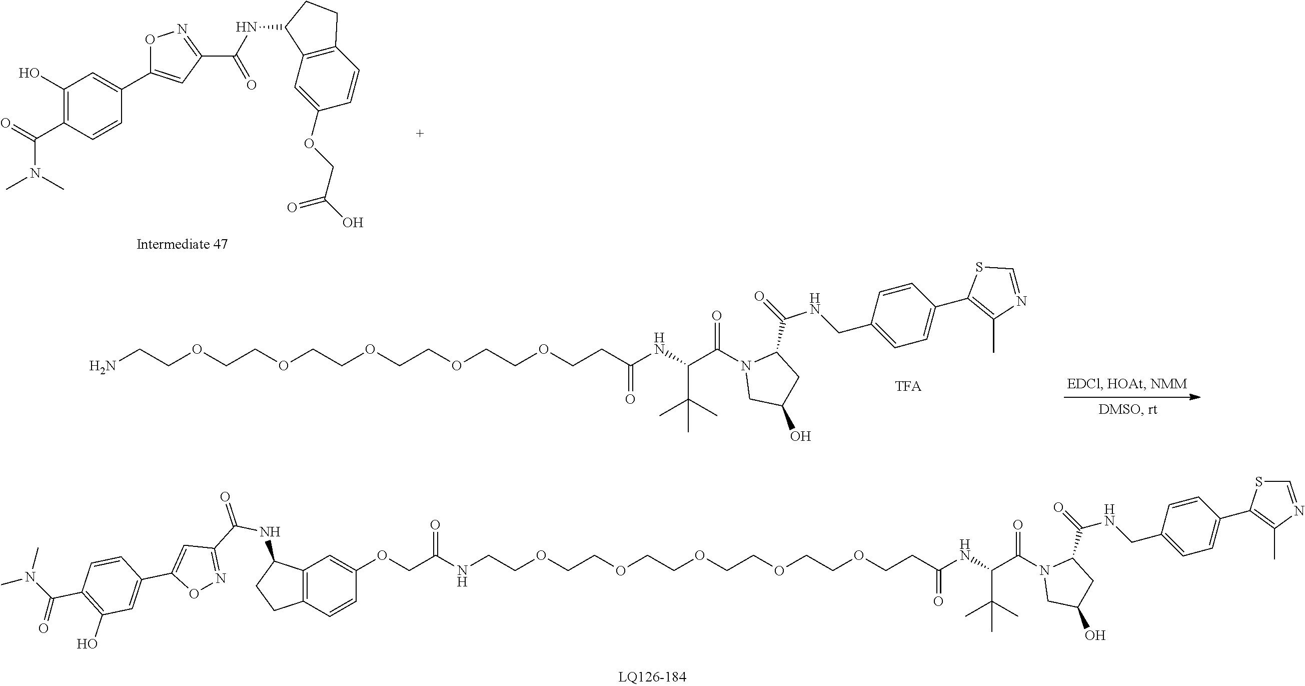

LQ126-184 was synthesized following the standard procedure for preparing LQ126-177 from intermediate 47 (5 mg, 0.01 mmol), (2S,4R)-1-((S)-1-amino-20-(tert-butyl)-18-oxo-3,6,9,12,15-pentaoxa-19-azahenicosan-21-oyl)-4-hydroxy-N-(4-(4-methylthiazol-5-yl)benzyl)pyrrolidine-2-carboxamide (8.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-184 was obtained as white solid (7.6 mg, 65%). ¹H NMR (600 MHz, Methanol-d₄) δ 9.30 (s, 1H), 7.57-7.41 (m, 5H), 7.39-7.33 (m, 2H), 7.24-7.15 (m, 2H), 6.99-6.87 (m, 2H), 5.62 (t, J=7.8 Hz, 1H), 4.66 (s, 1H), 4.61-4.47 (m, 5H), 4.37 (d, J=15.6 Hz, 1H), 3.90 (d, J=11.0 Hz, 1H), 3.81 (dd, J=11.0, 3.8 Hz, 1H), 3.75-3.68 (m, 2H), 3.67-3.52 (m, 20H), 3.49-3.42 (m, 2H), 3.19-2.92 (m, 7H), 2.92-2.83 (m, 1H), 2.66-2.44 (m, 4H), 2.27-2.20 (m, 1H), 2.14-2.06 (m, 2H), 1.04 (s, 9H). HRMS m/z [M+H]⁺ calcd for C₅₉H₇₇N₈O₁₅S⁺ 1169.5224, found 1169.5227.

### Example 283

### Synthesis of LQ126-185

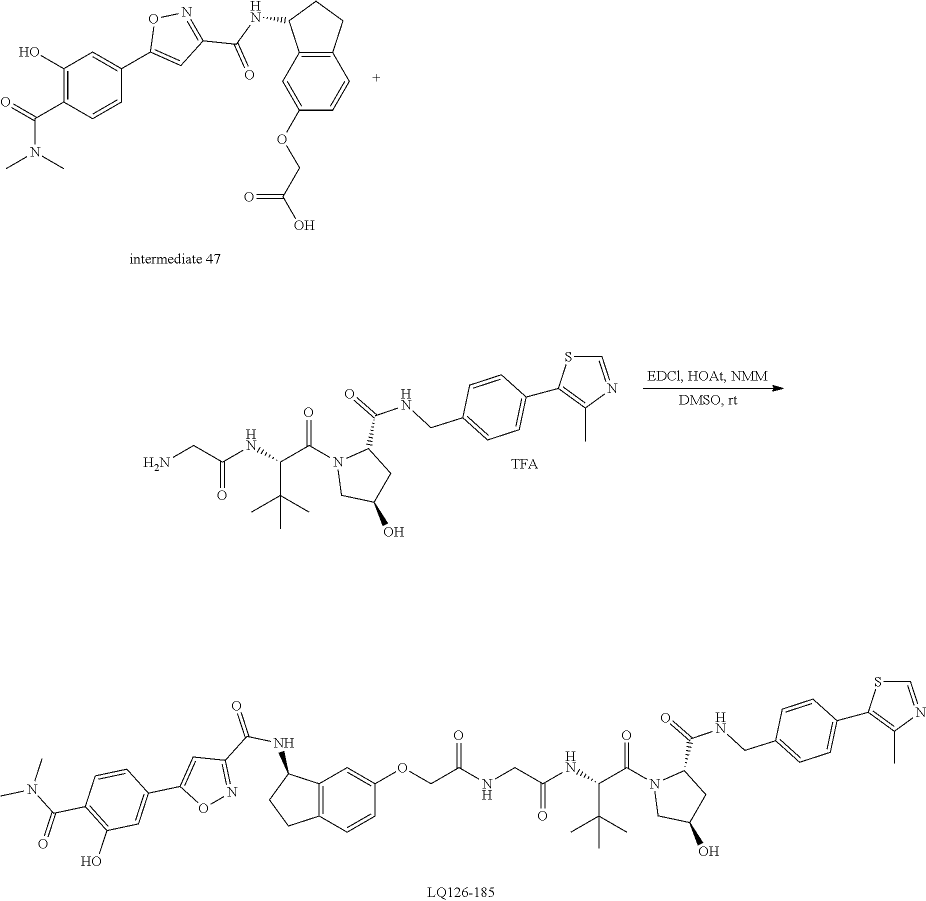

LQ126-185 was synthesized following the standard procedure for preparing LQ126-177 from intermediate 47 (5 mg, 0.01 mmol), (2S,4R)-1-((S)-2-(2-aminoacetamido)-3,3-dimethylbutanoyl)-4-hydroxy-N-(4-(4-methylthiazol-5-yl)benzyl)pyrrolidine-2-carboxamide (5.8 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-185 was obtained as white solid (5.9 mg, 63%). ¹H NMR (400 MHz, Methanol-d₄) δ 9.16 (s, 1H), 7.52-7.33 (m, 7H), 7.25-7.12 (m, 2H), 7.03-6.91 (m, 2H), 5.68-5.58 (m, 1H), 4.65 (d, J=5.7 Hz, 1H), 4.62-4.48 (m, 5H), 4.45-4.33 (m, 1H), 4.01 (s, 2H), 3.94-3.76 (m, 2H), 3.22-2.95 (m, 7H), 2.94-2.82 (m, 1H), 2.67-2.55 (m, 1H), 2.51 (s, 3H), 2.28-2.18 (m, 1H), 2.16-2.03 (m, 2H), 1.04 (s, 9H). HRMS m/z [M+H]⁺ calcd for C₄₈H₅₅N₈O₁₀S⁺ 935.3756, found 935.3755.

### Example 284

### Synthesis of LQ126-186

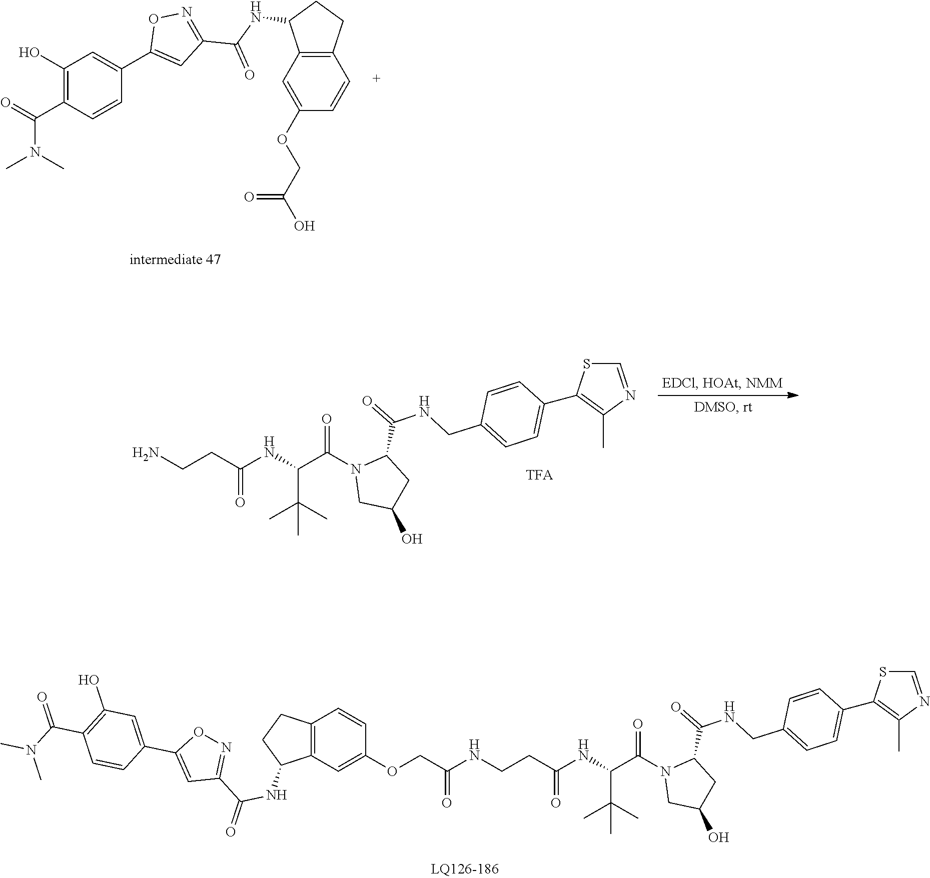

LQ126-186 was synthesized following the standard procedure for preparing LQ126-177 from intermediate 47 (5 mg, 0.01 mmol), (2S,4R)-1-((S)-2-(3-aminopropanamido)-3,3-dimethylbutanoyl)-4-hydroxy-N-(4-(4-methylthiazol-5-yl)benzyl)pyrrolidine-2-carboxamide (6 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ126-186 was obtained as white solid (6.7 mg, 71%). ¹H NMR (400 MHz, Methanol-d₄) δ 9.08 (s, 1H), 7.54-7.29 (m, 7H), 7.25-7.12 (m, 2H), 7.06-6.87 (m, 2H), 5.68-5.55 (m, 1H), 4.64-4.45 (m, 6H), 4.42-4.32 (m, 1H), 3.93 (d, J=10.7 Hz, 1H), 3.79 (d, J=11.5 Hz, 1H), 3.60-3.50 (m, 2H), 3.49-3.37 (m, 1H), 3.24-2.92 (m, 7H), 2.91-2.81 (m, 1H), 2.64-2.55 (m, 1H), 2.51 (s, 3H), 2.29-2.20 (m, 1H), 2.15-2.03 (m, 3H), 1.02 (s, 9H). HRMS m/z [M+H]⁺ calcd for C₄₉H₅₇N₈O₁₀S⁺ 949.3913, found 949.3919.

### Example 285

### Synthesis of LQ141-1

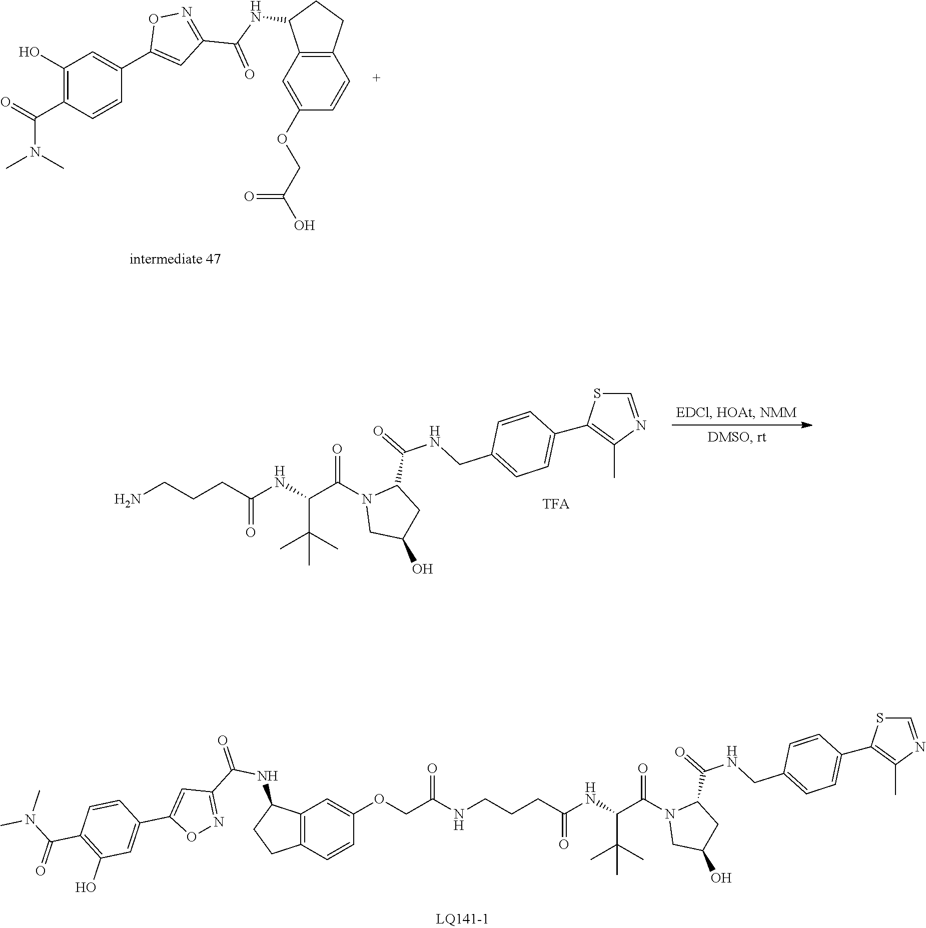

LQ141-1 was synthesized following the standard procedure for preparing LQ126-177 from intermediate 47 (5 mg, 0.01 mmol), (2S,4R)-1-((S)-2-(4-aminobutanamido)-3,3-dimethylbutanoyl)-4-hydroxy-N-(4-(4-methylthiazol-5-yl)benzyl)pyrrolidine-2-carboxamide (6.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-1 was obtained as white solid (7 mg, 73%). ¹H NMR (600 MHz, Methanol-d₄) δ 9.16 (s, 1H), 7.51-7.47 (m, 2H), 7.45-7.40 (m, 3H), 7.37 (d, J=1.6 Hz, 1H), 7.34 (d, J=7.9 Hz, 1H), 7.21 (d, J=8.3 Hz, 1H), 7.18 (s, 1H), 6.97-6.95 (m, 1H), 6.93-6.90 (m, 1H), 5.63 (t, J=7.9 Hz, 1H), 4.64-4.53 (m, 3H), 4.52-4.46 (m, 3H), 4.37 (d, J=15.6 Hz, 1H), 3.94-3.89 (m, 1H), 3.81 (dd, J=10.9, 3.9 Hz, 1H), 3.31-3.26 (m, 2H), 3.19-2.93 (m, 7H), 2.91-2.83 (m, 1H), 2.65-2.56 (m, 1H), 2.50 (s, 3H), 2.31-2.19 (m, 3H), 2.13-2.06 (m, 2H), 1.84-1.75 (m, 2H), 1.04 (s, 9H). HRMS m/z [M+H]⁺ calcd for C₅₀H₅₉N₈O₁₀S⁺ 963.4069, found 963.4061.

### Example 286

### Synthesis of LQ141-2

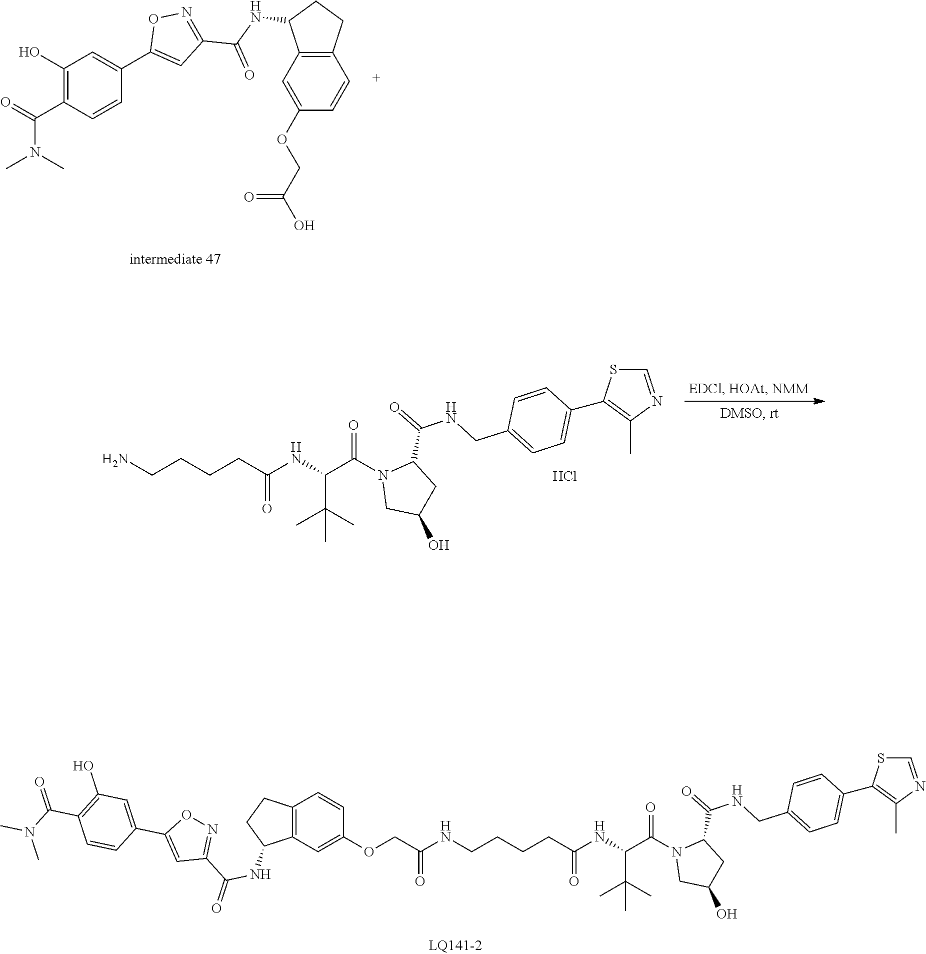

LQ141-2 was synthesized following the standard procedure for preparing LQ126-177 from intermediate 47 (5 mg, 0.01 mmol), (2S,4R)-1-((S)-2-(5-aminopentanamido)-3,3-dimethylbutanoyl)-4-hydroxy-N-(4-(4-methylthiazol-5-yl)benzyl)pyrrolidine-2-carboxamide (5.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-2 was obtained as white solid (6.8 mg, 70%). ¹H NMR (600 MHz, Methanol-d₄) δ 9.21-9.10 (m, 1H), 7.60-7.29 (m, 7H), 7.26-7.11 (m, 2H), 7.01-6.82 (m, 2H), 5.66-5.56 (m, 1H), 4.65-4.32 (m, 7H), 3.91 (d, J=11.0 Hz, 1H), 3.83-3.74 (m, 1H), 3.30-3.21 (m, 2H), 3.18-2.80 (m, 8H), 2.67-2.55 (m, 1H), 2.51 (s, 3H), 2.37-2.17 (m, 3H), 2.16-2.02 (m, 2H), 1.71-1.43 (m, 4H), 1.02 (s, 9H). HRMS m/z [M+H]⁺ calcd for C₅₁H₆₁N₈O₁₀S⁺ 977.4226, found 977.4189.

### Example 287

### Synthesis of LQ141-3

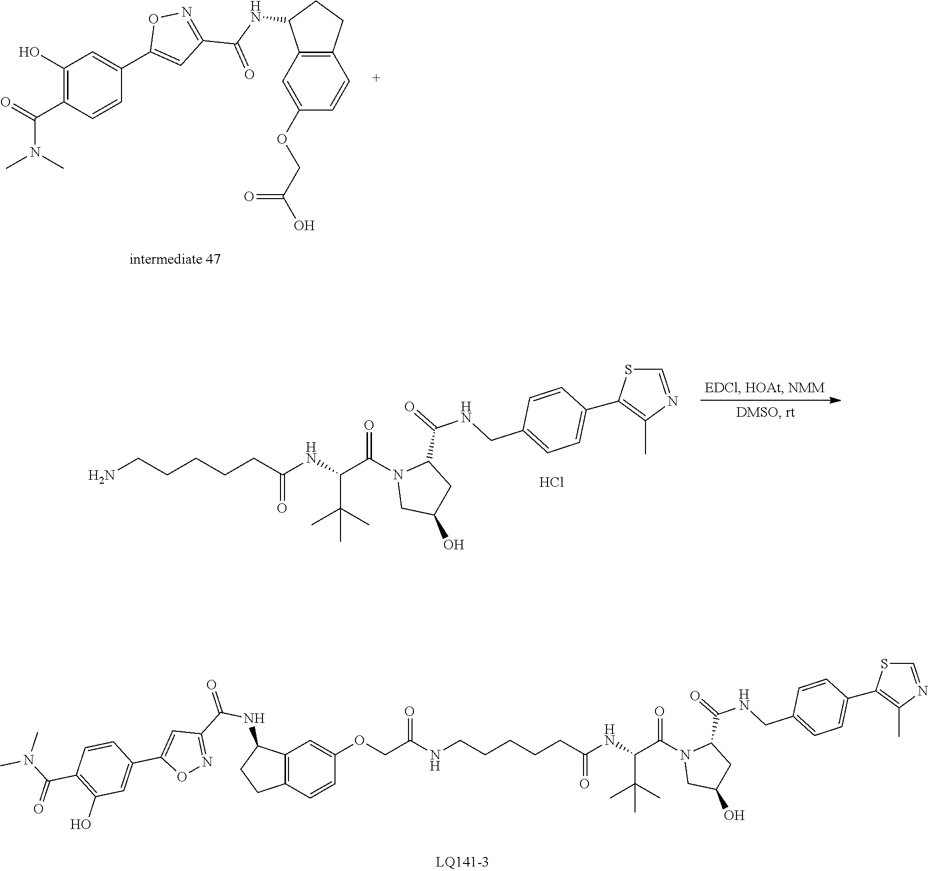

LQ141-3 was synthesized following the standard procedure for preparing LQ126-177 from intermediate 47 (5 mg, 0.01 mmol), (2S,4R)-1-((S)-2-(6-aminohexanamido)-3,3-dimethylbutanoyl)-4-hydroxy-N-(4-(4-methylthiazol-5-yl)benzyl)pyrrolidine-2-carboxamide (5.8 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-3 was obtained as white solid (6.6 mg, 67%). ¹H NMR (600 MHz, Methanol-d₄) δ 9.06 (s, 1H), 7.58-7.33 (m, 7H), 7.23-7.18 (m, 2H), 6.95-6.89 (m, 2H), 5.66-5.59 (m, 1H), 4.68-4.33 (m, 7H), 3.94-3.75 (m, 2H), 3.29-3.21 (m, 2H), 3.17-2.79 (m, 7H), 2.49 (s, 3H), 2.33-2.20 (m, 3H), 2.14-2.05 (m, 2H), 1.75-1.44 (m, 4H), 1.40-1.27 (m, 2H), 1.17-1.11 (m, 2H), 1.03 (s, 9H). HRMS m/z [M+H]⁺ calcd for C₅₂H₆₃N₈O₁₀S⁺ 991.4382, found 991.4363.

### Example 288

### Synthesis of LQ141-4

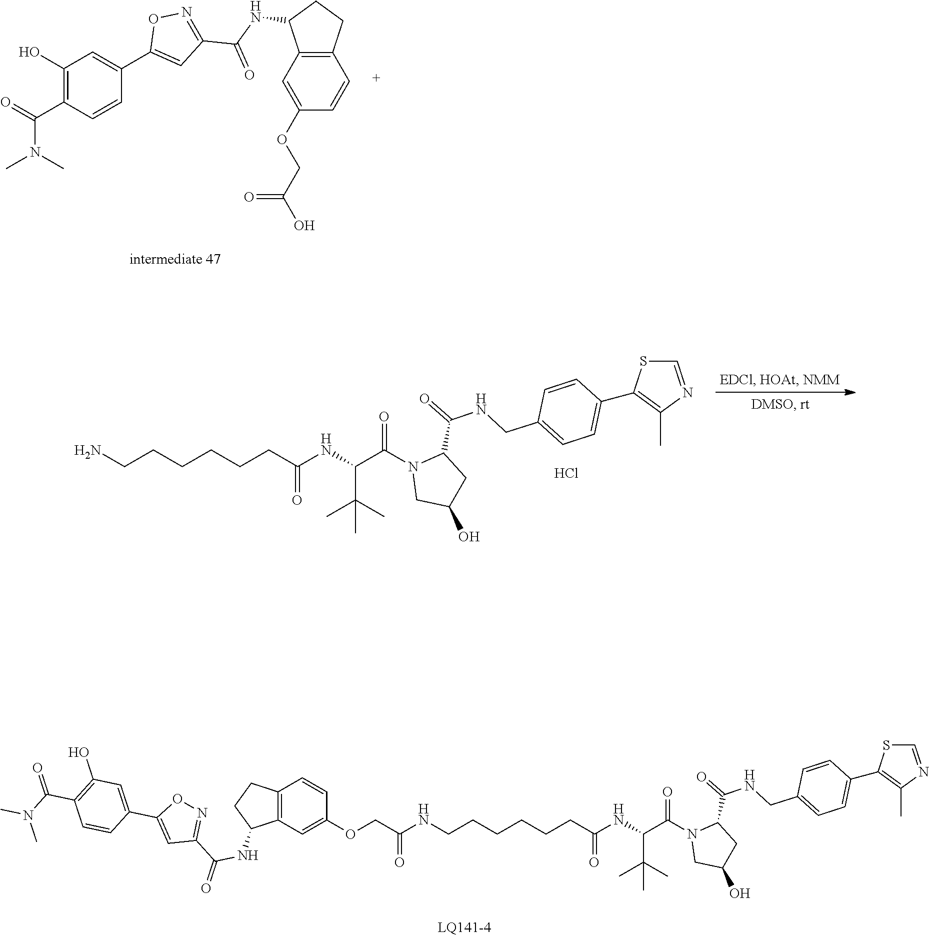

LQ141-4 was synthesized following the standard procedure for preparing LQ126-177 from intermediate 47 (5 mg, 0.01 mmol), (2S,4R)-1-((S)-2-(7-aminoheptanamido)-3,3-dimethylbutanoyl)-4-hydroxy-N-(4-(4-methylthiazol-5-yl)benzyl)pyrrolidine-2-carboxamide (5.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-4 was obtained as white solid (7.6 mg, 76%). ¹H NMR (600 MHz, Methanol-d₄) δ 9.16 (s, 1H), 7.50 (d, J=8.0 Hz, 2H), 7.46-7.42 (m, 3H), 7.37 (d, J=1.5 Hz, 1H), 7.35 (d, J=7.9 Hz, 1H), 7.20 (d, J=8.2 Hz, 1H), 7.17 (s, 1H), 6.94 (d, J=2.5 Hz, 1H), 6.89 (dd, J=8.2, 2.5 Hz, 1H), 5.61 (t, J=7.8 Hz, 1H), 4.64 (s, 1H), 4.62-4.53 (m, 2H), 4.52-4.49 (m, 1H), 4.47 (s, 2H), 4.37 (d, J=15.5 Hz, 1H), 3.92 (d, J=11.0 Hz, 1H), 3.81 (dd, J=11.0, 3.9 Hz, 1H), 3.24 (t, J=7.1 Hz, 2H), 3.17-2.92 (m, 7H), 2.91-2.83 (m, 1H), 2.65-2.56 (m, 1H), 2.51 (s, 3H), 2.32-2.20 (m, 3H), 2.14-2.04 (m, 2H), 1.62-1.54 (m, 2H), 1.54-1.46 (m, 2H), 1.34-1.25 (m, 4H), 1.04 (s, 9H). HRMS m/z [M+H]⁺ calcd for C₅₃H₆₅N₈O₁₀S⁺ 1005.4539, found 1005.4530.

### Example 289

### Synthesis of LQ141-5

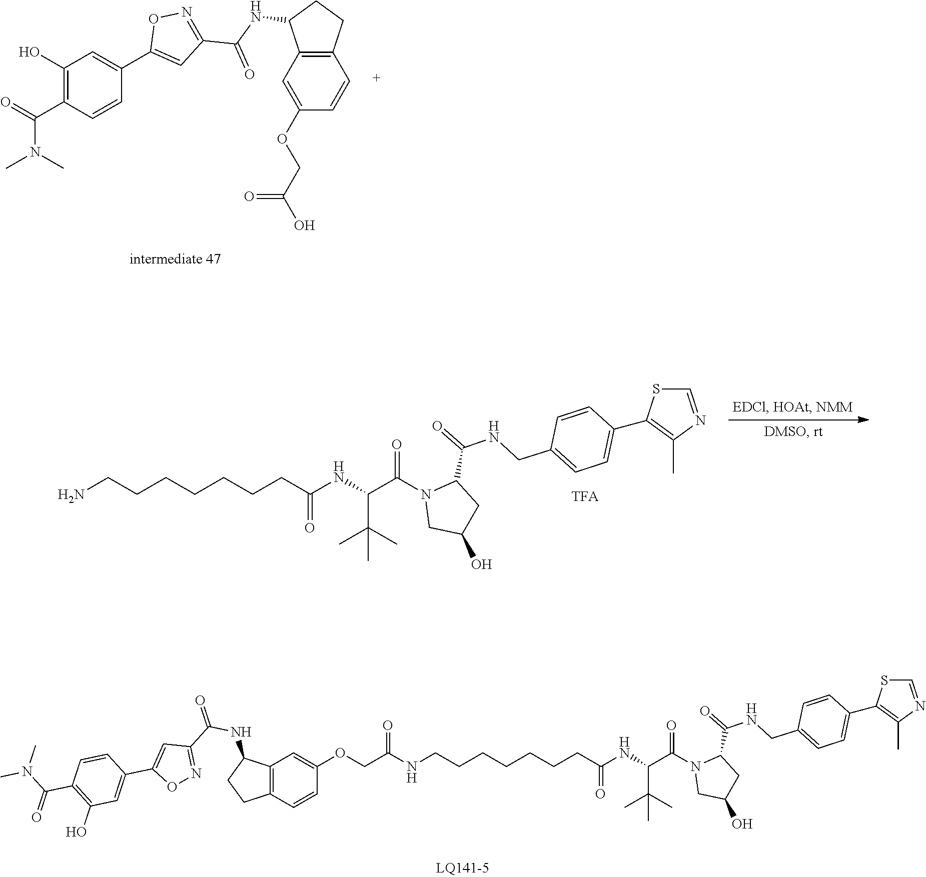

LQ141-5 was synthesized following the standard procedure for preparing LQ126-177 from intermediate 47 (5 mg, 0.01 mmol), (2S,4R)-1-((S)-2-(8-aminooctanamido)-3,3-dimethylbutanoyl)-4-hydroxy-N-(4-(4-methylthiazol-5-yl)benzyl)pyrrolidine-2-carboxamide (6.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-5 was obtained as white solid (7.1 mg, 70%). ¹H NMR (600 MHz, Methanol-d₄) δ 9.04 (s, 1H), 7.51-7.46 (m, 2H), 7.46-7.42 (m, 3H), 7.39-7.34 (m, 2H), 7.22-7.15 (m, 2H), 6.94 (d, J=2.5 Hz, 1H), 6.91-6.88 (m, 1H), 5.62 (t, J=7.8 Hz, 1H), 4.65 (s, 1H), 4.62-4.44 (m, 5H), 4.37 (d, J=15.5 Hz, 1H), 3.92 (d, J=10.9 Hz, 1H), 3.81 (dd, J=10.9, 3.9 Hz, 1H), 3.28-3.21 (m, 2H), 3.16-2.95 (m, 7H), 2.91-2.83 (m, 1H), 2.64-2.58 (m, 1H), 2.49 (s, 3H), 2.31-2.19 (m, 3H), 2.14-2.04 (m, 2H), 1.62-1.55 (m, 2H), 1.52-1.46 (m, 2H), 1.34-1.23 (m, 6H), 1.04 (s, 9H). HRMS m/z [M+H]⁺ calcd for C₅₄H₆₇N₈O₁₀S⁺ 1019.4695, found 1019.4702.

### Example 290

### Synthesis of LQ141-6

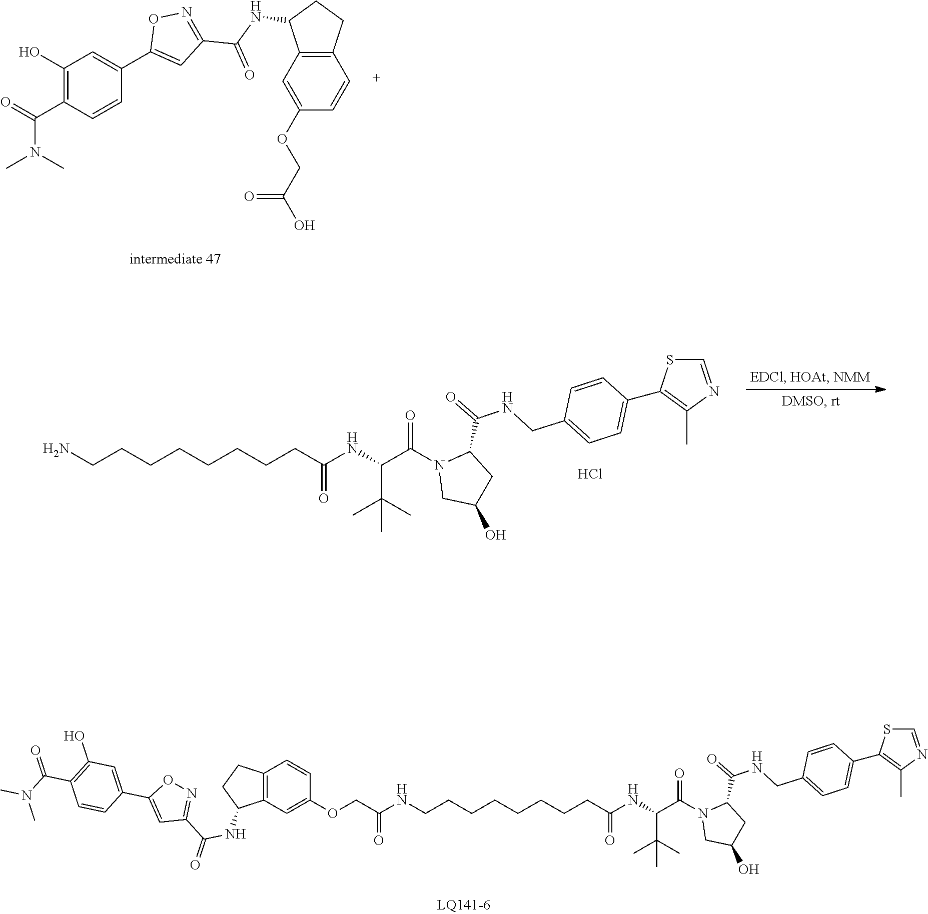

LQ141-6 was synthesized following the standard procedure for preparing LQ126-177 from intermediate 47 (5 mg, 0.01 mmol), (2S,4R)-1-((S)-2-(9-aminononanamido)-3,3-dimethylbutanoyl)-4-hydroxy-N-(4-(4-methylthiazol-5-yl)benzyl)pyrrolidine-2-carboxamide (6.2 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-6 was obtained as white solid (7.2 mg, 77%). ¹H NMR (600 MHz, Methanol-d₄) δ 9.21 (s, 1H), 7.50 (d, J=8.0 Hz, 2H), 7.47-7.41 (m, 3H), 7.39-7.33 (m, 2H), 7.20 (d, J=8.3 Hz, 1H), 7.17 (s, 1H), 6.94 (d, J=2.5 Hz, 1H), 6.90 (dd, J=8.3, 2.5 Hz, 1H), 5.61 (t, J=7.8 Hz, 1H), 4.65 (s, 1H), 4.62-4.45 (m, 5H), 4.38 (d, J=15.5 Hz, 1H), 3.92 (d, J=10.9 Hz, 1H), 3.81 (dd, J=11.0, 3.9 Hz, 1H), 3.24 (t, J=7.1 Hz, 2H), 3.17-2.94 (m, 7H), 2.91-2.82 (m, 1H), 2.65-2.57 (m, 1H), 2.51 (s, 3H), 2.34-2.20 (m, 3H), 2.13-2.06 (m, 2H), 1.64-1.56 (m, 2H), 1.53-1.44 (m, 2H), 1.38-1.22 (m, 8H), 1.04 (s, 9H). HRMS m/z [M+H]⁺ calcd for C₅₅H₆₉N₈O₁₀S⁺ 1033.4852, found 1033.4809.

### Example 291

### Synthesis of LQ141-7

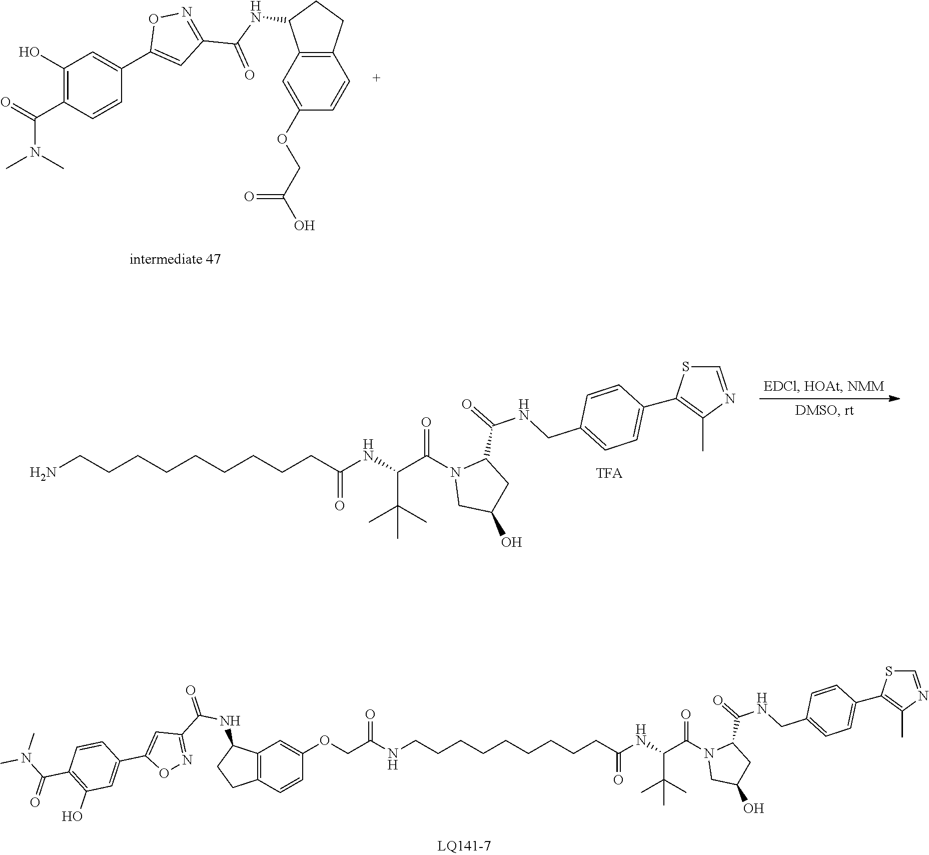

LQ141-7 was synthesized following the standard procedure for preparing LQ126-177 from intermediate 47 (5 mg, 0.01 mmol), (2S,4R)-1-((S)-2-(10-aminodecanamido)-3,3-dimethylbutanoyl)-4-hydroxy-N-(4-(4-methylthiazol-5-yl)benzyl)pyrrolidine-2-carboxamide (6.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-7 was obtained as white solid (7.6 mg, 73%). ¹H NMR (600 MHz, Methanol-d₄) δ 9.05 (s, 1H), 7.56-7.30 (m, 7H), 7.25-7.12 (m, 2H), 6.99-6.85 (m, 2H), 5.61 (t, J=7.8 Hz, 1H), 4.70-4.43 (m, 6H), 4.37 (d, J=15.7 Hz, 1H), 3.92 (d, J=10.9 Hz, 1H), 3.81 (d, J=10.5 Hz, 1H), 3.28-2.94 (m, 9H), 2.91-2.81 (m, 1H), 2.65-2.57 (m, 1H), 2.50 (s, 3H), 2.36-2.20 (m, 3H), 2.09 (t, J=10.7 Hz, 2H), 1.71-1.46 (m, 5H), 1.40-1.20 (m, 9H), 1.05 (s, 9H). HRMS m/z [M+H]⁺ calcd for C₅₆H₇₁N₈O₁₀S⁺ 1047.5008, found 1047.5000.

### Example 292

### Synthesis of LQ141-8

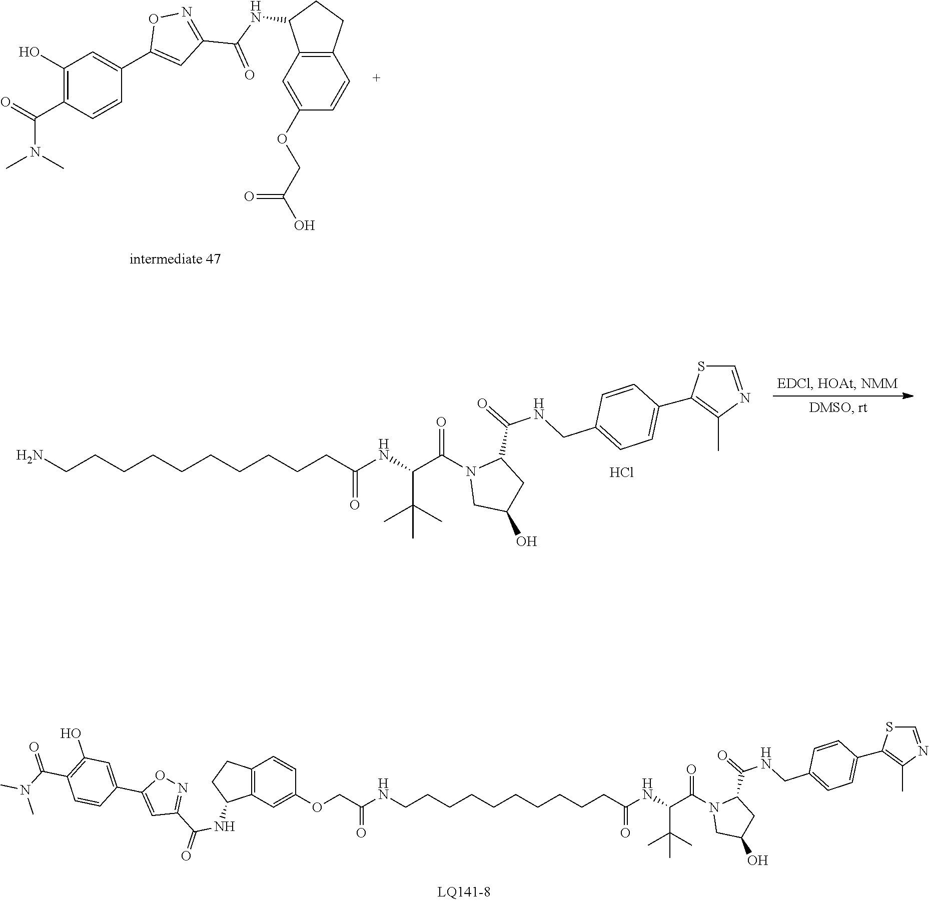

LQ141-8 was synthesized following the standard procedure for preparing LQ126-177 from intermediate 47 (5 mg, 0.01 mmol), (2S,4R)-1-((S)-2-(11-aminoundecanamido)-3,3-dimethylbutanoyl)-4-hydroxy-N-(4-(4-methylthiazol-5-yl)benzyl)pyrrolidine-2-carboxamide (6.5 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-8 was obtained as white solid (7 mg, 66%). ¹H NMR (600 MHz, Methanol-d₄) δ 9.15 (s, 1H), 7.62-7.30 (m, 7H), 7.26-7.11 (m, 2H), 7.02-6.84 (m, 2H), 5.67-5.56 (m, 1H), 4.72-4.31 (m, 7H), 4.02-3.76 (m, 2H), 3.28-3.19 (m, 2H), 3.18-2.80 (m, 9H), 2.68-2.56 (m, 2H), 2.51 (s, 3H), 2.40-2.01 (m, 5H), 1.70-1.42 (m, 5H), 1.40-1.20 (m, 11H), 1.04 (s, 9H). HRMS m/z [M+H]⁺ calcd for C₅₇H₇₃N₈O₁₀S⁺ 1061.5165, found 1061.5157.

### Example 293

### Synthesis of LQ141-9

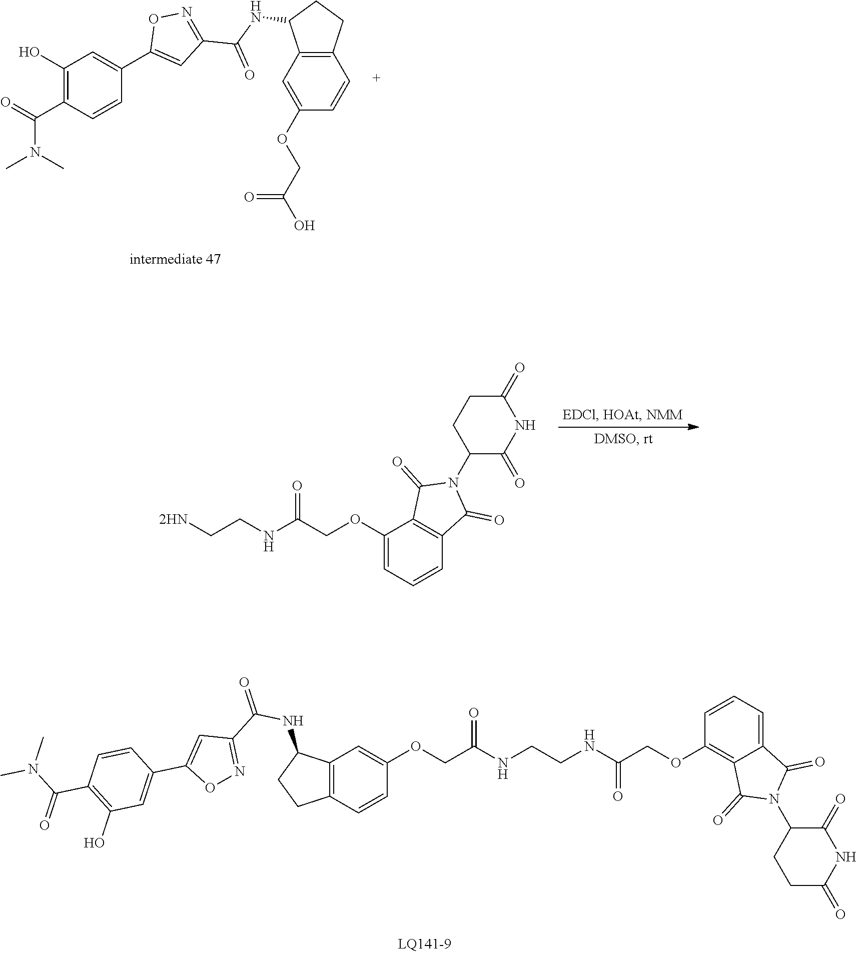

LQ141-9 was synthesized following the standard procedure for preparing LQ126-177 from intermediate 47 (5 mg, 0.01 mmol), N-(2-aminoethyl)-2-((2-(2,6-dioxopiperidin-3-yl)-1,3-dioxoisoindolin-4-yl)oxy)acetamide (4.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-9 was obtained as white solid (6.5 mg, 79%). ¹H NMR (600 MHz, Methanol-d₄) δ 7.76 (dd, J=8.4, 7.3 Hz, 1H), 7.48 (d, J=7.3 Hz, 1H), 7.42-7.36 (m, 2H), 7.35-7.32 (m, 2H), 7.16 (d, J=8.3 Hz, 1H), 7.12 (s, 1H), 6.95 (d, J=2.4 Hz, 1H), 6.86 (dd, J=8.3, 2.5 Hz, 1H), 5.59 (t, J=7.9 Hz, 1H), 5.01 (dd, J=12.2, 5.3 Hz, 1H), 4.69 (d, J=6.2 Hz, 2H), 4.47 (d, J=4.1 Hz, 2H), 3.52-3.43 (m, 4H), 3.17-2.93 (m, 7H), 2.89-2.81 (m, 1H), 2.72-2.57 (m, 4H), 2.13-2.04 (m, 2H). HRMS m/z [M+H]⁺ calcd for C₄₁H₄₀N₇O₁₂⁺ 822.2729, found 822.2716.

### Example 294

### Synthesis of LQ141-10

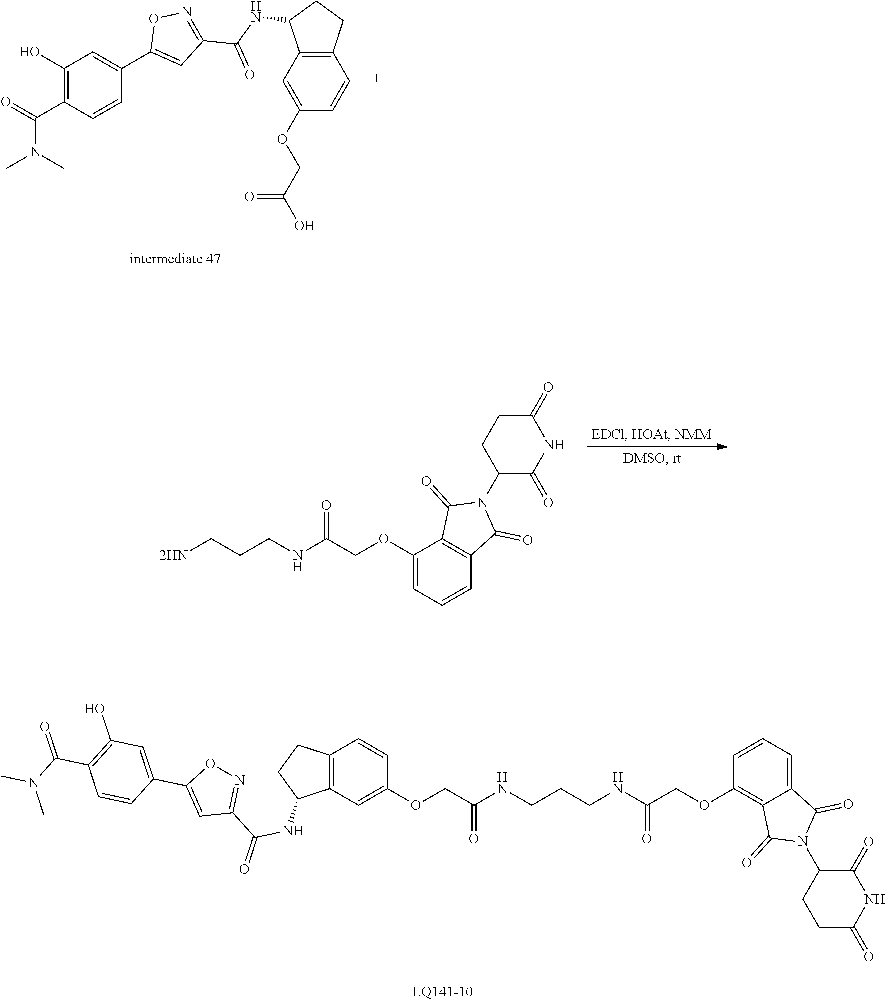

LQ141-10 was synthesized following the standard procedure for preparing LQ126-177 from intermediate 47 (5 mg, 0.01 mmol), N-(3-aminopropyl)-2-((2-(2,6-dioxopiperidin-3-yl)-1,3-dioxoisoindolin-4-yl)oxy)acetamide (5 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-10 was obtained as white solid (6.2 mg, 74%). ¹H NMR (600 MHz, Methanol-d₄) δ 7.78-7.73 (m, 1H), 7.47 (dd, J=7.3, 5.9 Hz, 1H), 7.41-7.36 (m, 2H), 7.35-7.30 (m, 2H), 7.16 (dd, J=8.4, 3.9 Hz, 1H), 7.12 (d, J=3.4 Hz, 1H), 6.95 (t, J=2.7 Hz, 1H), 6.89-6.85 (m, 1H), 5.59 (t, J=7.9 Hz, 1H), 5.12 (ddd, J=12.6, 7.6, 5.5 Hz, 1H), 4.77-4.69 (m, 2H), 4.47 (s, 2H), 3.38-3.33 (m, 1H), 3.32-3.25 (m, 2H), 3.18-2.95 (m, 8H), 2.89-2.79 (m, 2H), 2.77-2.68 (m, 2H), 2.63-2.56 (m, 1H), 2.18-2.05 (m, 2H), 1.80-1.73 (m, 2H). HRMS m/z [M+H]⁺ calcd for C₄₂H₄₂N₇O₁₂⁺ 836.2886, found 836.2856.

### Example 295

### Synthesis of LQ141-11

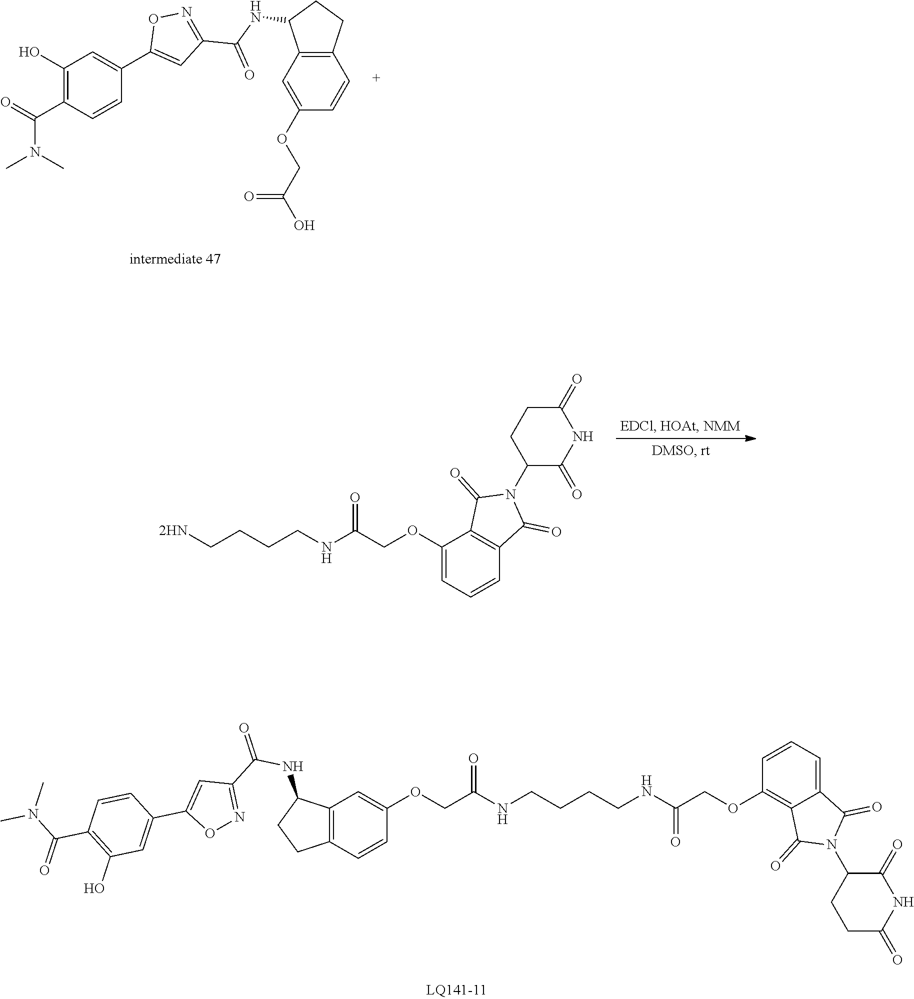

LQ141-11 was synthesized following the standard procedure for preparing LQ126-177 from intermediate 47 (5 mg, 0.01 mmol), N-(4-aminobutyl)-2-((2-(2,6-dioxopiperidin-3-yl)-1,3-dioxoisoindolin-4-yl)oxy)acetamide (5.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-11 was obtained as white solid (5.9 mg, 70%). ¹H NMR (600 MHz, Methanol-d₄) δ 7.82-7.74 (m, 1H), 7.55-7.29 (m, 5H), 7.24-7.06 (m, 2H), 7.02-6.83 (m, 2H), 5.64-5.54 (m, 1H), 5.14-5.07 (m, 1H), 4.75-4.70 (m, 2H), 4.51-4.45 (m, 2H), 3.33-3.23 (m, 4H), 3.18-2.93 (m, 8H), 2.89-2.65 (m, 3H), 2.62-2.56 (m, 1H), 2.17-2.02 (m, 2H), 1.64-1.49 (m, 4H). HRMS m/z [M+H]⁺ calcd for C₄₃H₄₄N₇O₁₂⁺ 850.3042, found 850.3041.

### Example 296

### Synthesis of LQ141-12

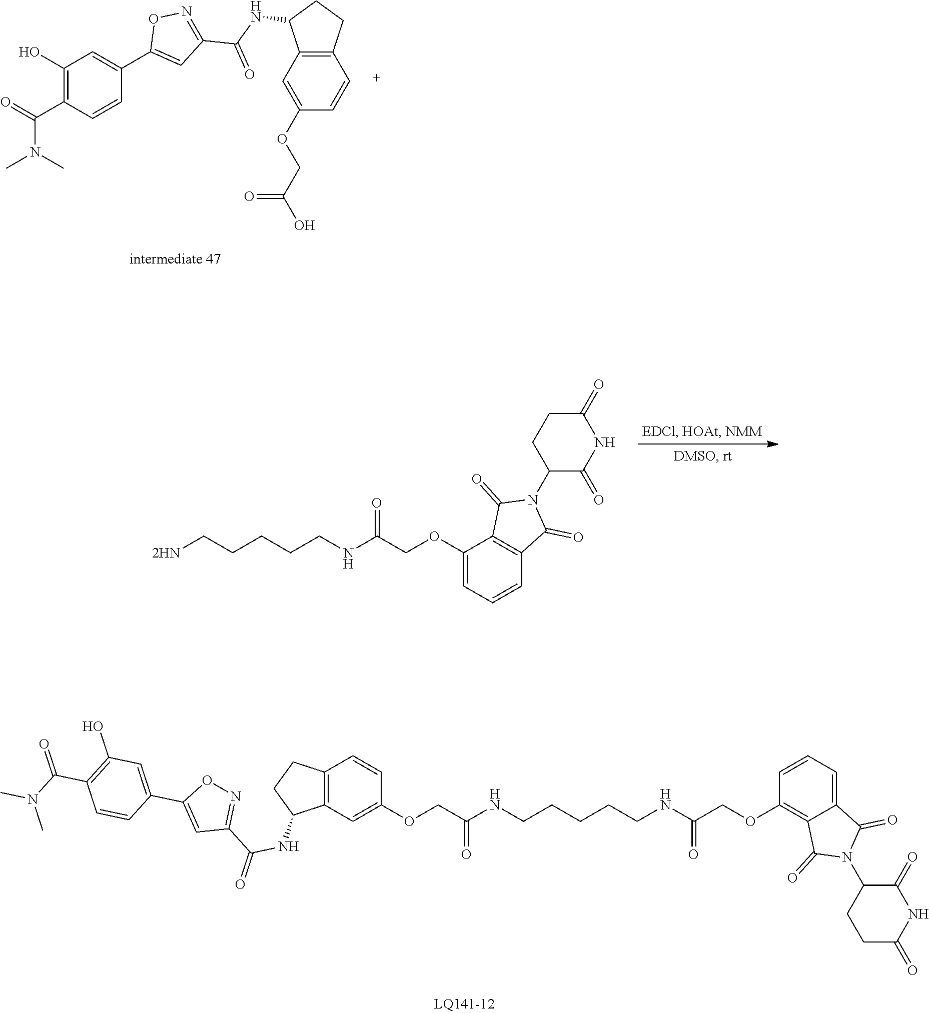

LQ141-12 was synthesized following the standard procedure for preparing LQ126-177 from intermediate 47 (5 mg, 0.01 mmol), N-(5-aminopentyl)-2-((2-(2,6-dioxopiperidin-3-yl)-1,3-dioxoisoindolin-4-yl)oxy)acetamide (5.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-12 was obtained as white solid (6.4 mg, 74%). ¹H NMR (600 MHz, Methanol-d₄) δ 7.83-7.70 (m, 1H), 7.55-7.23 (m, 5H), 7.22-7.08 (m, 2H), 7.02-6.75 (m, 2H), 5.71-5.50 (m, 1H), 5.28-5.10 (m, 1H), 4.72 (s, 2H), 4.50-4.40 (m, 2H), 3.31-2.54 (m, 16H), 2.23-2.01 (m, 2H), 1.69-1.26 (m, 6H). HRMS m/z [M+H]⁺ calcd for C₄₄H₄₆N₇O₁₂⁺ 864.3199, found 864.3194.

### Example 297

### Synthesis of LQ141-13

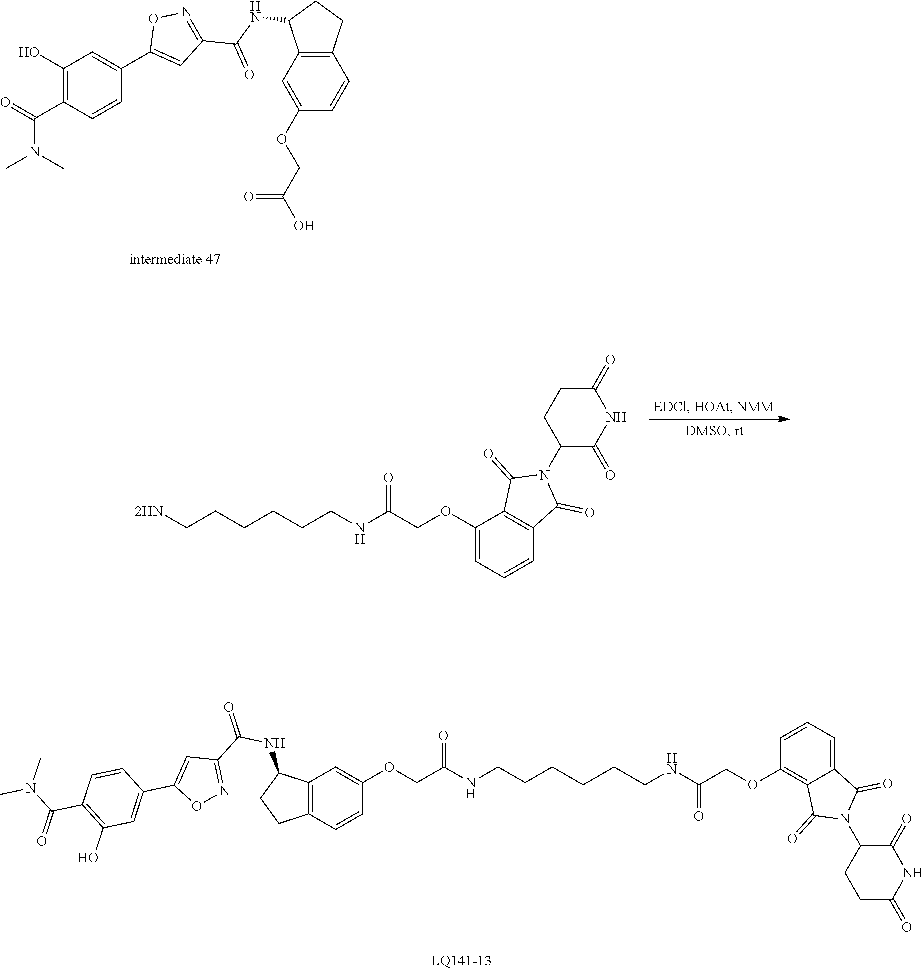

LQ141-13 was synthesized following the standard procedure for preparing LQ126-177 from intermediate 47 (5 mg, 0.01 mmol), N-(6-aminohexyl)-2-((2-(2,6-dioxopiperidin-3-yl)-1,3-dioxoisoindolin-4-yl)oxy)acetamide (5.4 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-13 was obtained as white solid (5.9 mg, 67%). ¹H NMR (600 MHz, Methanol-d₄) δ 7.84-7.74 (m, 1H), 7.55-7.29 (m, 5H), 7.23-7.09 (m, 2H), 7.00-6.84 (m, 2H), 5.60 (t, J=7.9 Hz, 1H), 5.17-5.05 (m, 1H), 4.73 (s, 2H), 4.50-4.42 (m, 2H), 3.30-3.19 (m, 3H), 3.18-2.53 (m, 13H), 2.23-2.00 (m, 2H), 1.61-1.20 (m, 8H). HRMS m/z [M+H]⁺ calcd for C₄₅H₄₈N₇O₁₂⁺ 878.3355, found 878.3357.

### Example 298

### Synthesis of LQ141-14

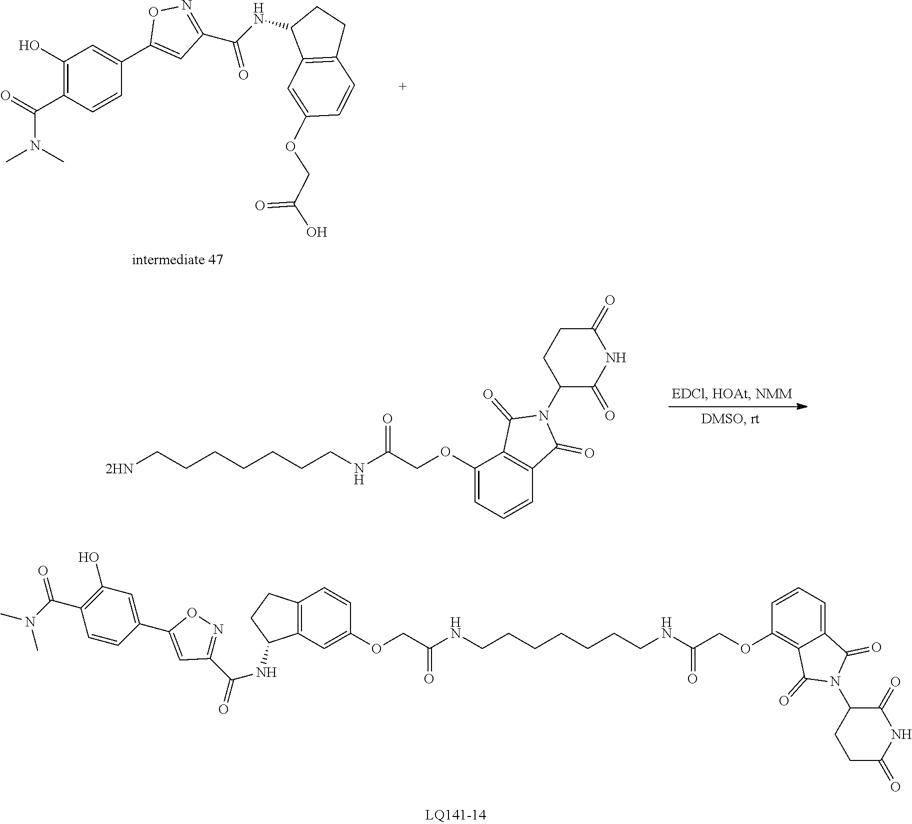

LQ141-14 was synthesized following the standard procedure for preparing LQ126-177 from intermediate 47 (5 mg, 0.01 mmol), N-(7-aminoheptyl)-2-((2-(2,6-dioxopiperidin-3-yl)-1,3-dioxoisoindolin-4-yl)oxy)acetamide (5.6 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-14 was obtained as white solid (5.6 mg, 63%). ¹H NMR (600 MHz, Methanol-d₄) δ 7.82-7.77 (m, 1H), 7.51 (d, J=7.3 Hz, 1H), 7.44-7.39 (m, 2H), 7.37-7.32 (m, 2H), 7.19 (d, J=8.3 Hz, 1H), 7.14 (d, J=2.2 Hz, 1H), 6.96 (d, J=2.4 Hz, 1H), 6.89 (dd, J=8.3, 2.5 Hz, 1H), 5.64-5.58 (m, 1H), 5.14 (dd, J=12.5, 5.5 Hz, 1H), 4.75 (s, 2H), 4.47 (d, J=2.4 Hz, 2H), 3.30 (t, J=6.7 Hz, 2H), 3.23 (t, J=7.1 Hz, 2H), 3.16-2.94 (m, 8H), 2.91-2.83 (m, 2H), 2.78-2.70 (m, 2H), 2.64-2.58 (m, 1H), 2.18-2.11 (m, 1H), 2.11-2.04 (m, 1H), 1.58-1.52 (m, 2H), 1.51-1.45 (m, 2H), 1.37-1.23 (m, 5H). HRMS m/z [M+H]⁺ calcd for C₄₆H₅₀N₇O₁₂⁺ 892.3512, found 892.3510.

### Example 299

### Synthesis of LQ141-15

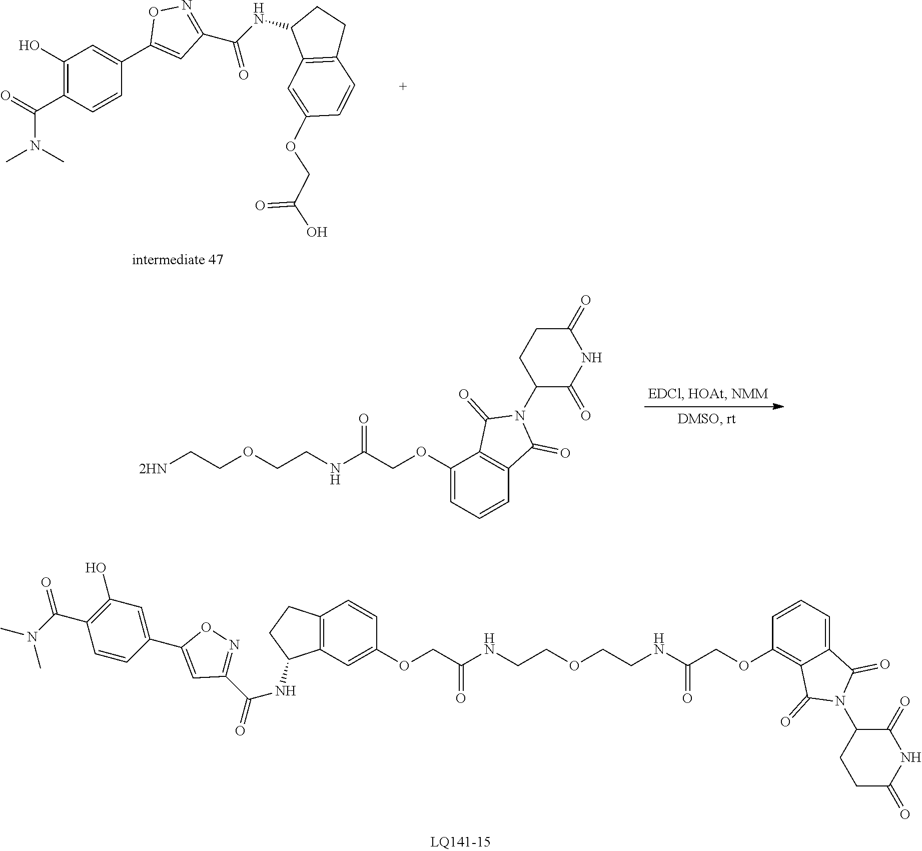

LQ141-15 was synthesized following the standard procedure for preparing LQ126-177 from intermediate 47 (5 mg, 0.01 mmol), N-(2-(2-aminoethoxy)ethyl)-2-((2-(2,6-dioxopiperidin-3-yl)-1,3-dioxoisoindolin-4-yl)oxy)acetamide (5.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-15 was obtained as white solid (5.9 mg, 68%). ¹H NMR (600 MHz, Methanol-d₄) δ 7.79-7.68 (m, 1H), 7.52-7.26 (m, 4H), 7.19-7.03 (m, 2H), 6.98-6.86 (m, 2H), 6.85-6.70 (m, 1H), 5.60-5.48 (m, 1H), 5.15-5.08 (m, 1H), 4.74-4.63 (m, 2H), 4.43 (s, 2H), 3.70-3.38 (m, 7H), 3.23-2.91 (m, 8H), 2.88-2.53 (m, 5H), 2.22-2.01 (m, 2H). HRMS m/z [M+H]⁺ calcd for C₄₃H₄₄N₇O₁₃⁺ 866.2992, found 866.2989.

### Example 300

### Synthesis of LQ141-16

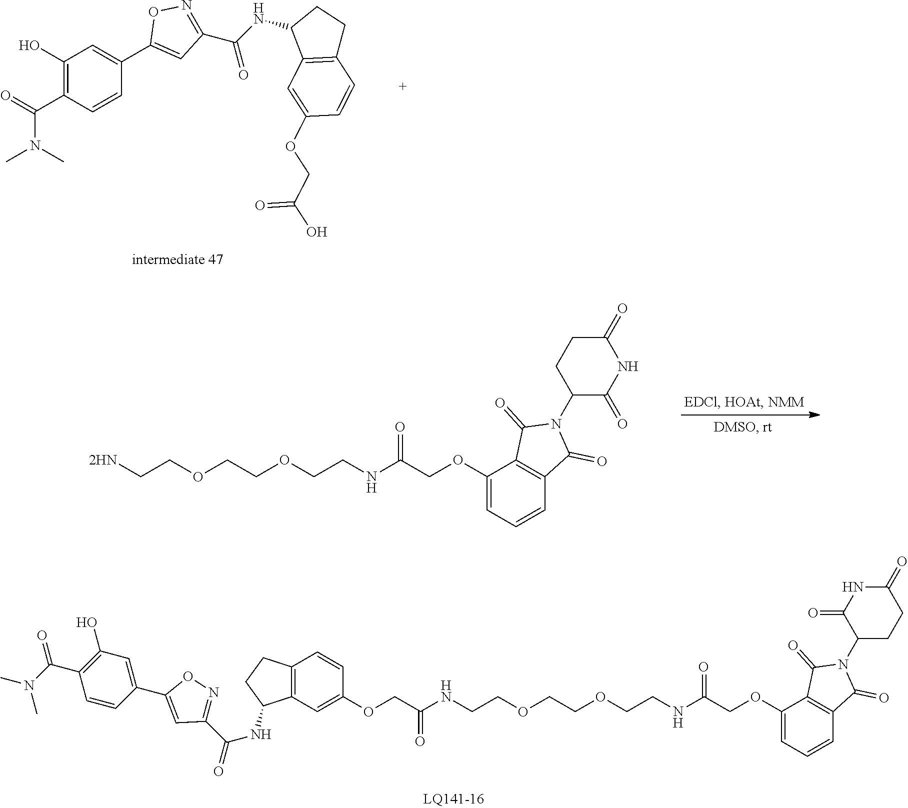

LQ141-16 was synthesized following the standard procedure for preparing LQ126-177 from intermediate 47 (5 mg, 0.01 mmol), N-(2-(2-(2-aminoethoxy)ethoxy)ethyl)-2-((2-(2,6-dioxopiperidin-3-yl)-1,3-dioxoisoindolin-4-yl)oxy)acetamide (5.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-16 was obtained as white solid (6.5 mg, 72%). ¹H NMR (600 MHz, Methanol-d₄) δ 7.82-7.70 (m, 1H), 7.51-7.28 (m, 4H), 7.24-7.10 (m, 3H), 6.99-6.80 (m, 2H), 5.64-5.55 (m, 1H), 5.17-5.08 (m, 1H), 4.70 (s, 2H), 4.45 (s, 2H), 3.71-3.38 (m, 11H), 3.20-2.94 (m, 8H), 2.91-2.80 (m, 2H), 2.73 (t, J=15.2 Hz, 2H), 2.64-2.54 (m, 1H), 2.21-2.02 (m, 2H). HRMS m/z [M+H]⁺ calcd for C₄₅H₄₈N₇O₁₄⁺ 910.3254, found 910.3217.

### Example 301

### Synthesis of LQ141-17

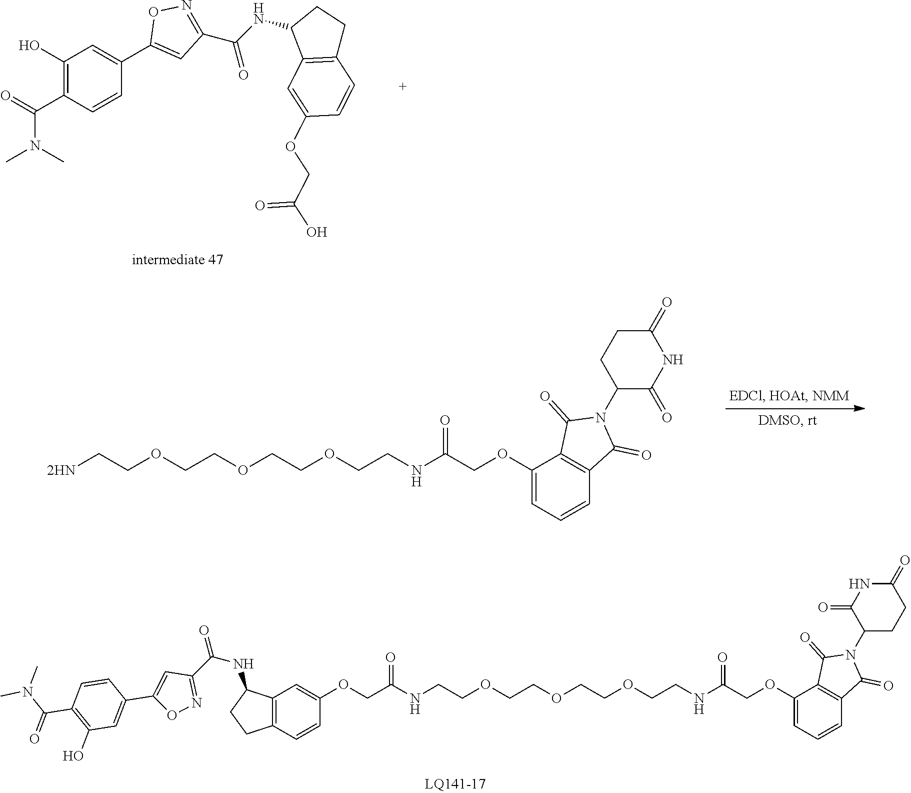

LQ141-17 was synthesized following the standard procedure for preparing LQ126-177 from intermediate 47 (5 mg, 0.01 mmol), N-(2-(2-(2-(2-aminoethoxy)ethoxy)ethoxy)ethyl)-2-((2-(2,6-dioxopiperidin-3-yl)-1,3-dioxoisoindolin-4-yl)oxy)acetamide (6.2 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-17 was obtained as white solid (6.3 mg, 66%). ¹H NMR (600 MHz, Methanol-d₄) δ 7.81-7.75 (m, 1H), 7.49 (dd, J=7.3, 1.7 Hz, 1H), 7.42-7.37 (m, 2H), 7.36-7.31 (m, 2H), 7.20-7.16 (m, 1H), 7.14 (d, J=2.6 Hz, 1H), 6.96 (t, J=3.1 Hz, 1H), 6.90-6.85 (m, 1H), 5.60 (t, J=7.9 Hz, 1H), 5.11 (ddd, J=12.7, 5.5, 2.1 Hz, 1H), 4.73 (s, 2H), 4.48 (s, 2H), 3.63-3.52 (m, 11H), 3.49-3.45 (m, 2H), 3.43 (t, J=5.4 Hz, 2H), 3.16-2.95 (m, 8H), 2.90-2.82 (m, 2H), 2.79-2.68 (m, 2H), 2.63-2.57 (m, 1H), 2.18-2.12 (m, 1H), 2.12-2.05 (m, 1H). HRMS m/z [M+H]⁺ calcd for C₄₇H₅₂N₇O₁₅⁺ 954.3516, found 954.3493.

### Example 302

### Synthesis of LQ141-18

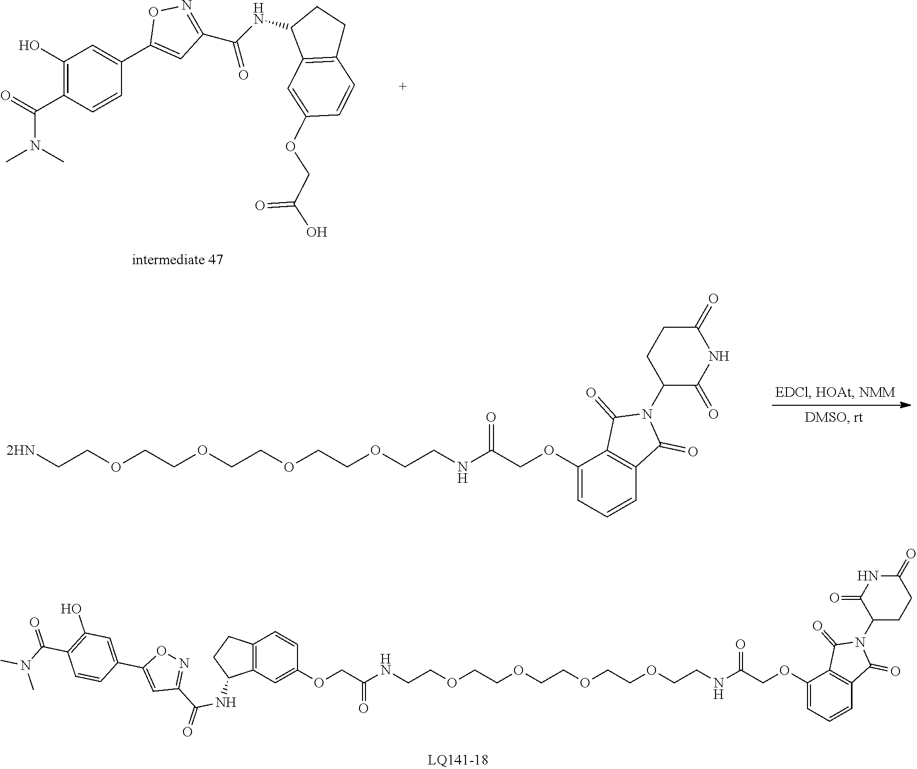

LQ141-18 was synthesized following the standard procedure for preparing LQ126-177 from intermediate 47 (5 mg, 0.01 mmol), N-(14-amino-3,6,9,12-tetraoxatetradecyl)-2-((2-(2,6-dioxopiperidin-3-yl)-1,3-dioxoisoindolin-4-yl)oxy)acetamide (6.6 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-18 was obtained as white solid (6.9 mg, 69%). ¹H NMR (600 MHz, Methanol-d₄) δ 7.81-7.75 (m, 1H), 7.50 (dd, J=7.3, 2.8 Hz, 1H), 7.44-7.30 (m, 4H), 7.25-7.10 (m, 2H), 6.99-6.84 (m, 2H), 5.60 (t, J=7.8 Hz, 1H), 5.11 (dd, J=13.1, 5.4 Hz, 1H), 4.74 (s, 2H), 4.48 (s, 2H), 3.73-3.39 (m, 21H), 3.20-2.95 (m, 8H), 2.91-2.83 (m, 2H), 2.79-2.67 (m, 2H), 2.65-2.54 (m, 1H), 2.22-2.04 (m, 2H). HRMS m/z [M+H]⁺ calcd for C₄₉H₅₆N₇O₁₆⁺ 998.3778, found 998.3761.

### Example 303

### Synthesis of LQ141-19

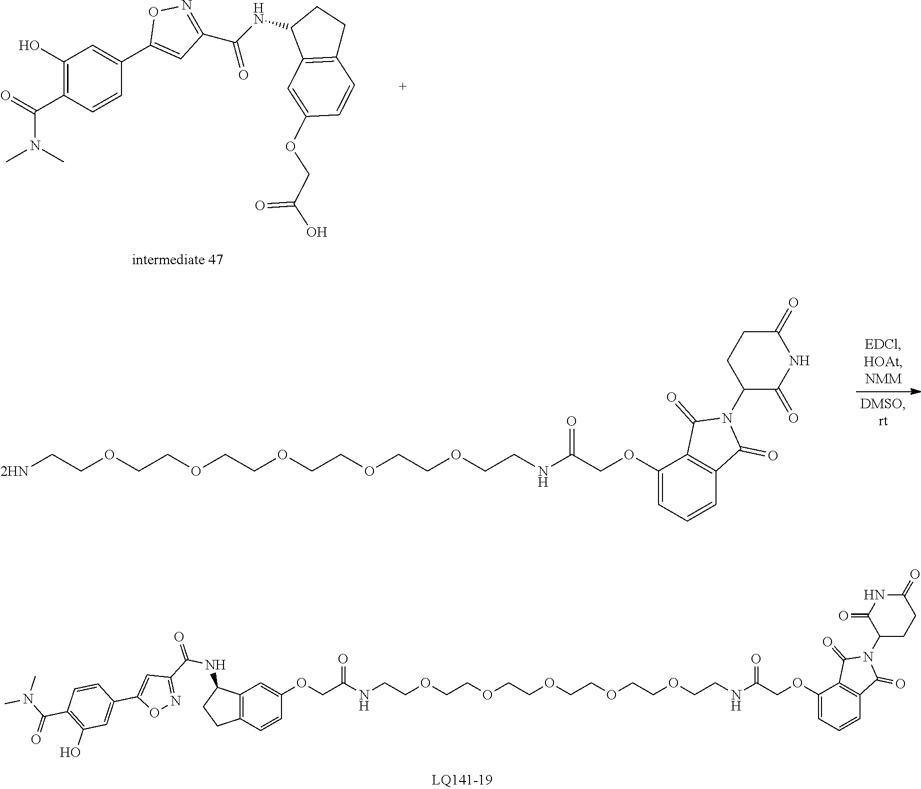

LQ141-19 was synthesized following the standard procedure for preparing LQ126-177 from intermediate 47 (5 mg, 0.01 mmol), N-(17-amino-3,6,9,12,15-pentaoxaheptadecyl)-2-((2-(2,6-dioxopiperidin-3-yl)-1,3-dioxoisoindolin-4-yl)oxy)acetamide (7.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-19 was obtained as white solid (7.6 mg, 73%). ¹H NMR (600 MHz, Methanol-d₄) δ 7.82-7.76 (m, 1H), 7.50 (d, J=7.3 Hz, 1H), 7.44-7.39 (m, 2H), 7.37-7.32 (m, 2H), 7.20 (d, J=8.3 Hz, 1H), 7.18-7.14 (m, 1H), 6.97-6.94 (m, 1H), 6.91-6.85 (m, 1H), 5.61 (t, J=7.9 Hz, 1H), 5.12 (dd, J=12.9, 5.5 Hz, 1H), 4.75 (s, 2H), 4.55-4.44 (m, 2H), 3.72-3.41 (m, 23H), 3.20-2.95 (m, 8H), 2.92-2.83 (m, 2H), 2.81-2.67 (m, 2H), 2.65-2.57 (m, 1H), 2.20-2.13 (m, 1H), 2.12-2.05 (m, 1H). HRMS m/z [M+H]⁺ calcd for C₅₁H₆₀N₇O₁₇⁺ 1042.4040, found 1042.4023.

### Example 304

### Synthesis of LQ141-20

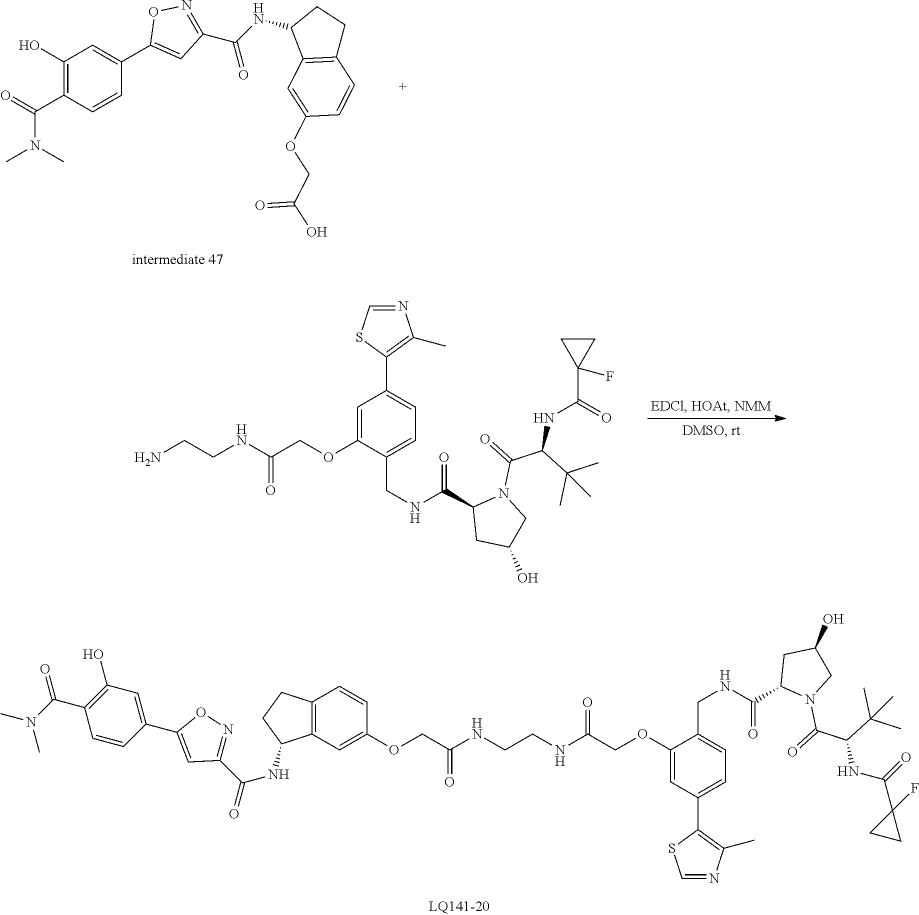

LQ141-20 was synthesized following the standard procedure for preparing LQ126-177 from intermediate 47 (5 mg, 0.01 mmol), (2S,4R)—N-(2-(2-((2-aminoethyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (7.5 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-20 was obtained as white solid (7 mg, 65%). ¹H NMR (600 MHz, Methanol-d₄) δ 9.14 (s, 1H), 7.50 (d, J=7.8 Hz, 1H), 7.45 (dd, J=9.4, 3.3 Hz, 1H), 7.42-7.39 (m, 1H), 7.37-7.31 (m, 2H), 7.18-7.15 (m, 2H), 7.08 (dd, J=7.7, 1.6 Hz, 1H), 6.97 (d, J=1.7 Hz, 1H), 6.92 (d, J=2.5 Hz, 1H), 6.86 (dd, J=8.2, 2.5 Hz, 1H), 5.59 (t, J=8.0 Hz, 1H), 4.72 (d, J=8.6 Hz, 1H), 4.62-4.56 (m, 2H), 4.54-4.44 (m, 3H), 4.35 (d, J=1.9 Hz, 2H), 3.84 (d, J=11.1 Hz, 1H), 3.79 (dd, J=11.1, 3.8 Hz, 1H), 3.52-3.41 (m, 4H), 3.17-2.94 (m, 8H), 2.87-2.79 (m, 1H), 2.63-2.55 (m, 1H), 2.49 (s, 3H), 2.21-2.14 (m, 1H), 2.11-2.02 (m, 2H), 1.40-1.22 (m, 4H), 1.00 (s, 9H). HRMS m/z [M+H]⁺ calcd for C₅₄H₆₃FN₉O₁₂S⁺ 1080.4295, found 1080.4245.

### Example 305

### Synthesis of LQ141-21

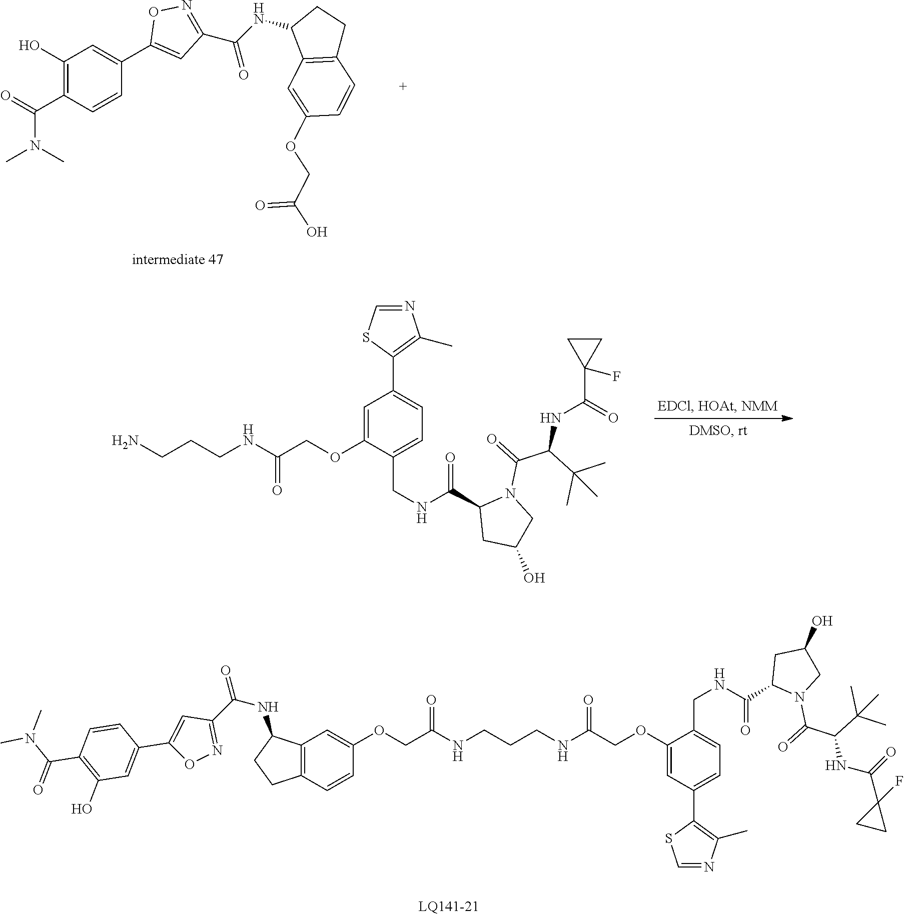

LQ141-21 was synthesized following the standard procedure for preparing LQ126-177 from intermediate 47 (5 mg, 0.01 mmol), (2S,4R)—N-(2-(2-((3-aminopropyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (7.6 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-21 was obtained as white solid (7.5 mg, 69%). ¹H NMR (600 MHz, Methanol-d₄) δ 9.09 (s, 1H), 7.58-7.27 (m, 5H), 7.25-7.04 (m, 3H), 7.01-6.85 (m, 3H), 5.59 (t, J=8.1 Hz, 1H), 4.77-4.39 (m, 8H), 3.87-3.71 (m, 2H), 3.40-3.22 (m, 4H), 3.20-2.81 (m, 8H), 2.63-2.54 (m, 1H), 2.48 (s, 3H), 2.24-2.00 (m, 3H), 1.80-1.67 (m, 2H), 1.41-1.16 (m, 6H), 0.99 (s, 9H). HRMS m/z [M+H]⁺ calcd for C₅₅H₆₅FN₉O₁₂S⁺ 1094.4452, found 1094.4426.

### Example 306

### Synthesis of LQ141-22

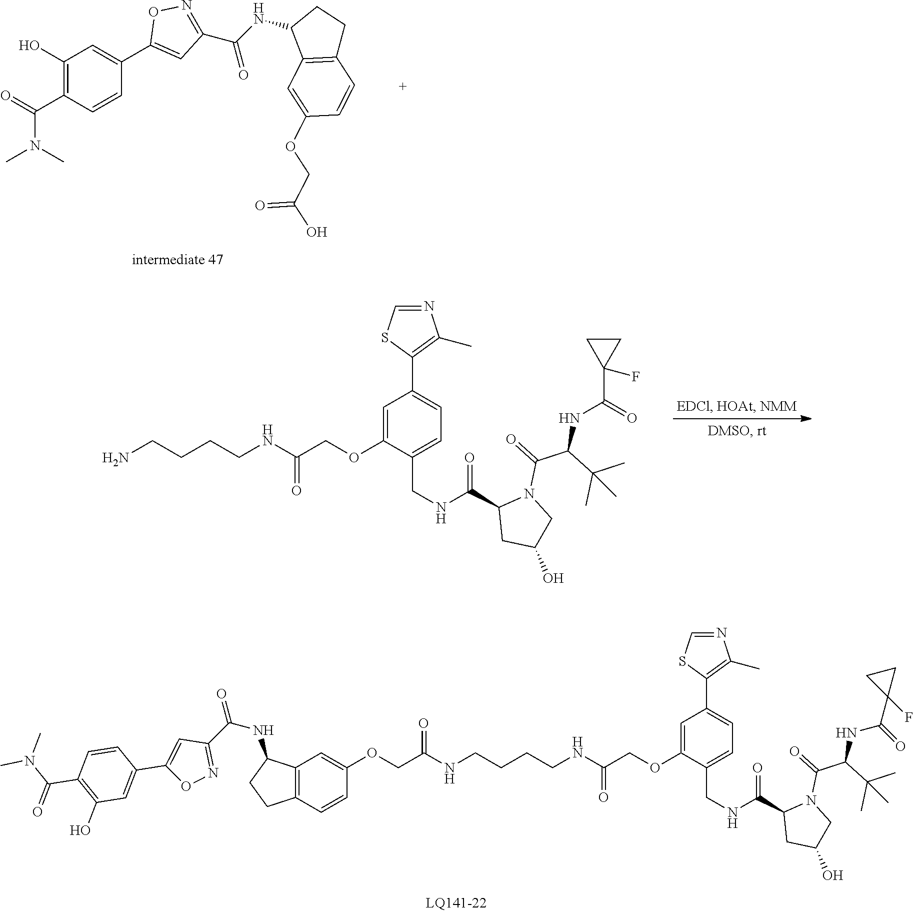

LQ141-22 was synthesized following the standard procedure for preparing LQ126-177 from intermediate 47 (5 mg, 0.01 mmol), (2S,4R)—N-(2-(2-((4-aminobutyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (7.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-22 was obtained as white solid (7.1 mg, 64%). ¹H NMR (600 MHz, Methanol-d₄) δ 9.11 (s, 1H), 7.53-7.45 (m, 2H), 7.44-7.40 (m, 1H), 7.38-7.32 (m, 2H), 7.21 (d, J=8.3 Hz, 1H), 7.15 (d, J=1.5 Hz, 1H), 7.10 (dd, J=10.0, 3.8 Hz, 1H), 7.02-6.86 (m, 3H), 5.62 (t, J=7.9 Hz, 1H), 4.73 (d, J=9.3 Hz, 1H), 4.66-4.44 (m, 7H), 3.84 (d, J=11.0 Hz, 1H), 3.78 (dd, J=11.4, 4.0 Hz, 1H), 3.31-3.21 (m, 4H), 3.18-2.93 (m, 8H), 2.91-2.83 (m, 1H), 2.64-2.57 (m, 1H), 2.50 (s, 3H), 2.23-2.17 (m, 1H), 2.13-2.04 (m, 2H), 1.64-1.46 (m, 4H), 1.42-1.23 (m, 4H), 1.02 (s, 9H). HRMS m/z [M+H]⁺ calcd for C₅₆H₆₇FN₉O₁₂S⁺ 1108.4608, found 1108.4599.

### Example 307

### Synthesis of LQ141-24

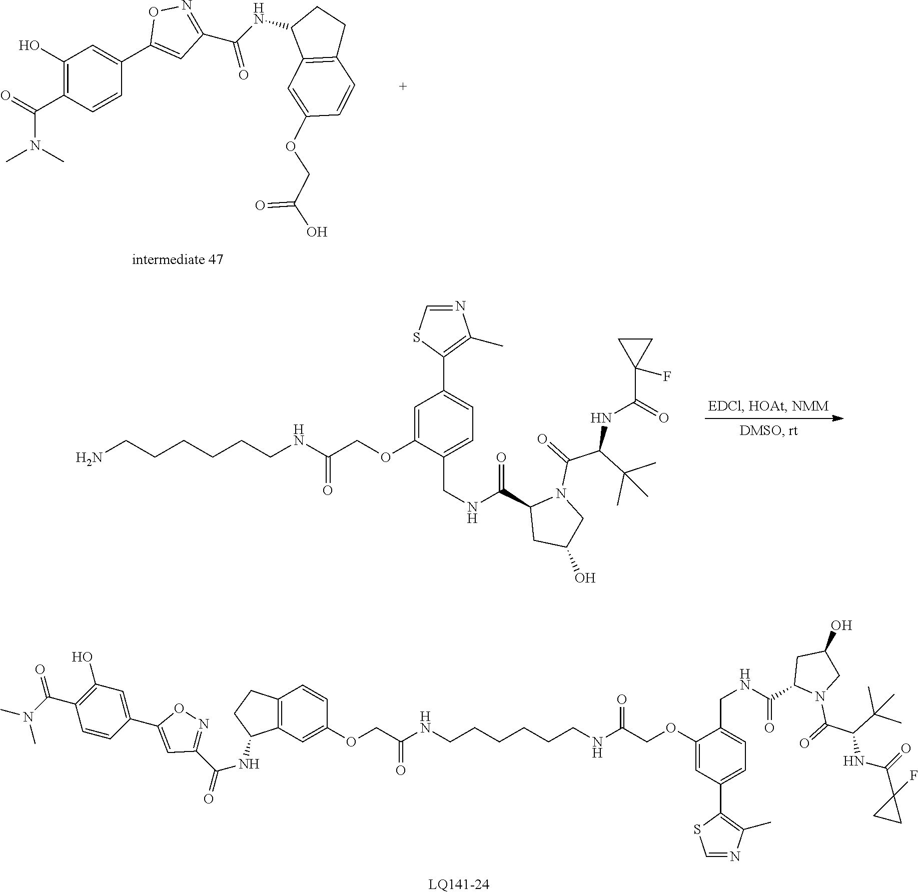

LQ141-24 was synthesized following the standard procedure for preparing LQ126-177 from intermediate 47 (5 mg, 0.01 mmol), (2S,4R)—N-(2-(2-((6-aminohexyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (8 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-24 was obtained as white solid (6.8 mg, 60%). ¹H NMR (600 MHz, Methanol-d₄) δ 9.05 (s, 1H), 7.54-7.31 (m, 5H), 7.21 (d, J=8.3 Hz, 1H), 7.17-7.13 (m, 1H), 7.12-7.08 (m, 1H), 7.01-6.96 (m, 2H), 6.90 (dd, J=8.3, 2.6 Hz, 1H), 5.62 (t, J=7.8 Hz, 1H), 4.74 (d, J=9.2 Hz, 1H), 4.67-4.55 (m, 3H), 4.51-4.45 (m, 4H), 3.85 (d, J=10.9 Hz, 1H), 3.78 (dd, J=11.1, 3.8 Hz, 1H), 3.29-3.25 (m, 2H), 3.21 (t, J=7.1 Hz, 2H), 3.17-2.95 (m, 8H), 2.91-2.83 (m, 1H), 2.65-2.57 (m, 1H), 2.50 (s, 3H), 2.25-2.18 (m, 1H), 2.14-2.04 (m, 2H), 1.60-1.42 (m, 4H), 1.40-1.21 (m, 8H), 1.02 (s, 9H). HRMS m/z [M+H]⁺ calcd for C₅₈H₇₁FN₉O₁₂S⁺ 1136.4921, found 1136.4898.

### Example 308

### Synthesis of LQ141-26

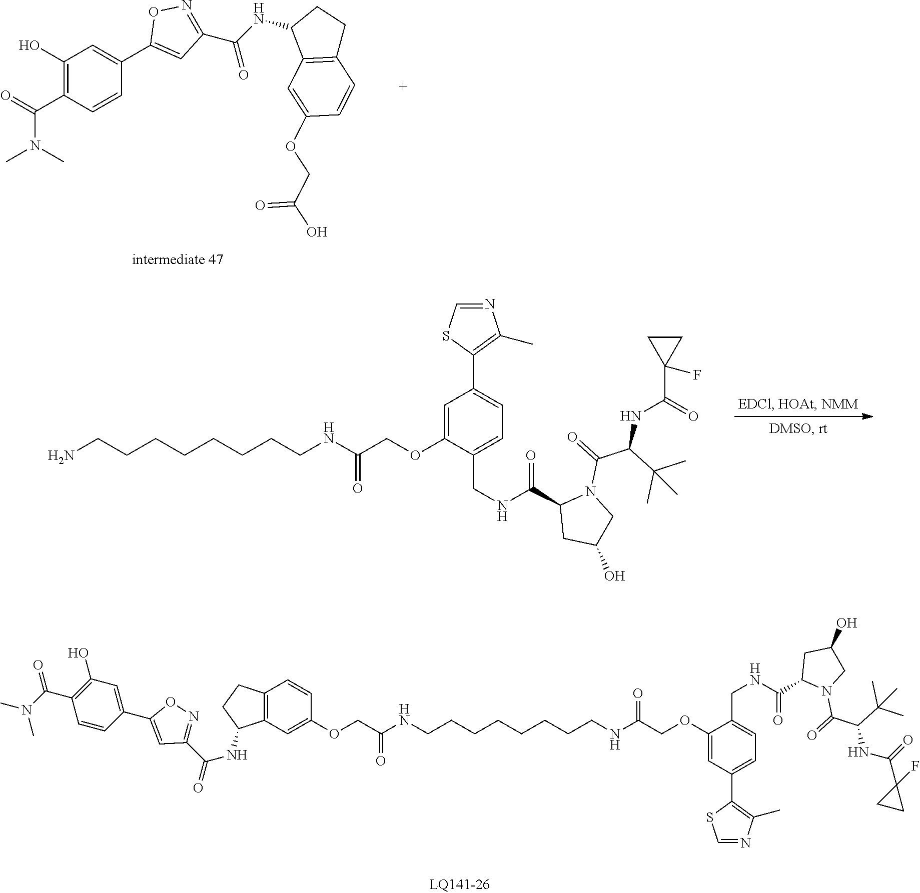

LQ141-26 was synthesized following the standard procedure for preparing LQ126-177 from intermediate 47 (5 mg, 0.01 mmol), (2S,4R)—N-(2-(2-((8-aminooctyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (8.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-26 was obtained as white solid (8.1 mg, 70%). ¹H NMR (600 MHz, Methanol-d₄) δ 9.00 (s, 1H), 7.55-7.31 (m, 5H), 7.21 (d, J=8.3 Hz, 1H), 7.17-7.08 (m, 2H), 7.01-6.94 (m, 1H), 6.90 (dd, J=8.4, 2.4 Hz, 1H), 5.62 (t, J=7.8 Hz, 1H), 4.75 (d, J=9.3 Hz, 1H), 4.66-4.56 (m, 3H), 4.51-4.45 (m, 4H), 3.86 (d, J=11.1 Hz, 1H), 3.79 (dd, J=11.1, 3.8 Hz, 1H), 3.28 (t, J=7.0 Hz, 2H), 3.23 (t, J=7.2 Hz, 2H), 3.18-2.94 (m, 8H), 2.91-2.83 (m, 1H), 2.65-2.57 (m, 1H), 2.50 (s, 3H), 2.25-2.19 (m, 1H), 2.14-2.04 (m, 2H), 1.58-1.44 (m, 6H), 1.42-1.19 (m, 10H), 1.02 (s, 9H). HRMS m/z [M+H]⁺ calcd for C₆₀H₇₅FN₉O₁₂S⁺ 1164.5234, found 1164.5180.

### Example 309

### Synthesis of LQ141-27

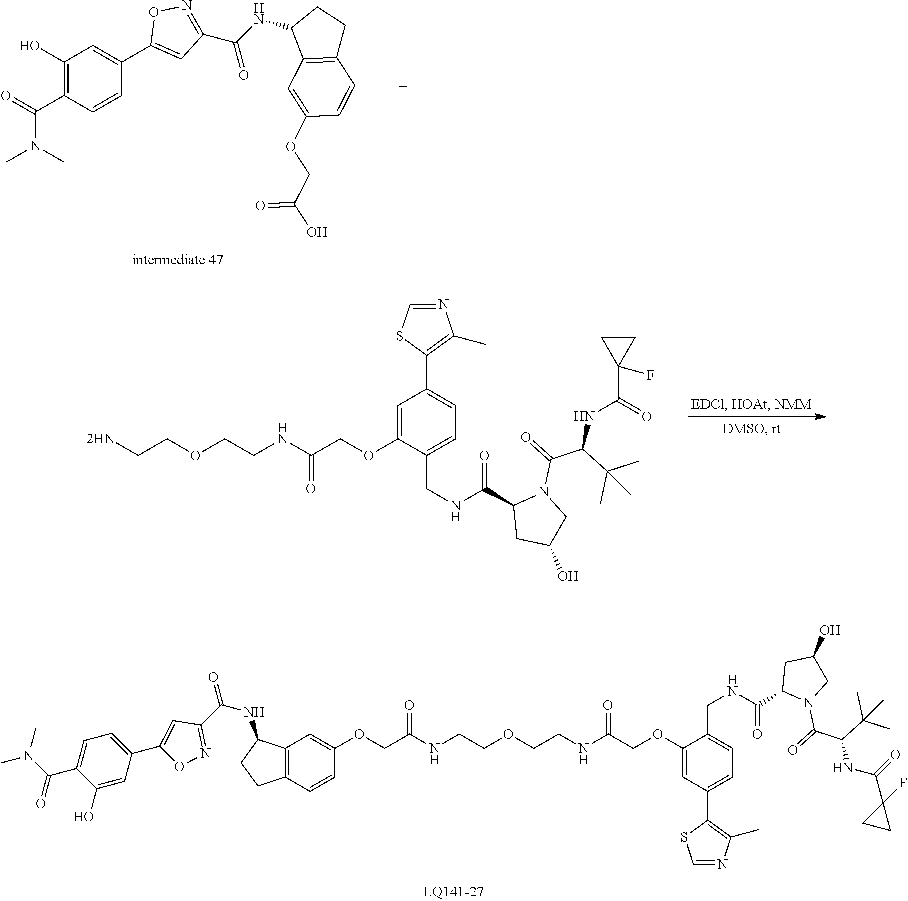

LQ141-27 was synthesized following the standard procedure for preparing LQ126-177 from intermediate 47 (5 mg, 0.01 mmol), (2S,4R)—N-(2-(2-((2-(2-aminoethoxy)ethyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (7.9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-27 was obtained as white solid (8.1 mg, 72%). ¹H NMR (600 MHz, Methanol-d₄) δ 9.08 (s, 1H), 7.55-7.29 (m, 5H), 7.22-7.05 (m, 3H), 7.00-6.82 (m, 3H), 5.63-5.54 (m, 1H), 4.78-4.42 (m, 8H), 3.90-3.73 (m, 2H), 3.69-3.36 (m, 8H), 3.20-2.94 (m, 8H), 2.89-2.80 (m, 1H), 2.66-2.56 (m, 1H), 2.48 (s, 3H), 2.25-2.16 (m, 1H), 2.15-2.04 (m, 2H), 1.42-1.19 (m, 4H), 1.02 (s, 9H). HRMS m/z [M+H]⁺ calcd for C₅₆H₆₇FN₉O₁₃S⁺ 1124.4558, found 1124.4572.

### Example 310

### Synthesis of LQ141-28

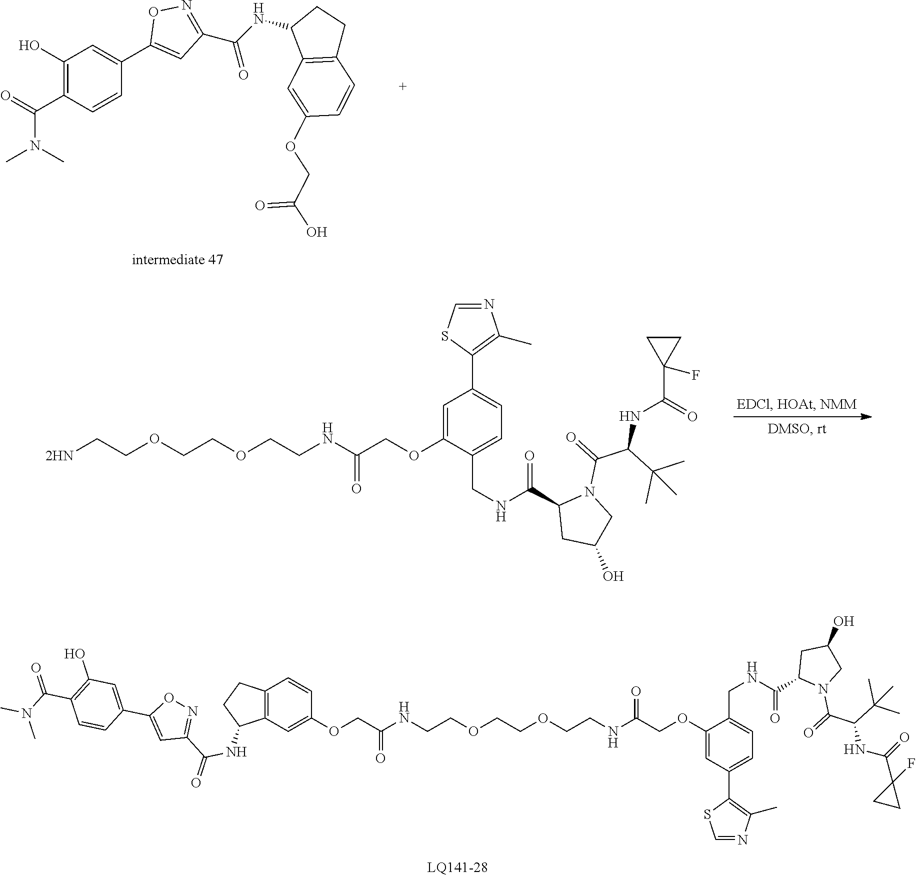

LQ141-28 was synthesized following the standard procedure for preparing LQ126-177 from intermediate 47 (5 mg, 0.01 mmol), (2S,4R)—N-(2-(2-((2-(2-(2-aminoethoxy)ethoxy)ethyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (8.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-28 was obtained as white solid (7.9 mg, 68%). ¹H NMR (600 MHz, Methanol-d₄) δ 9.05 (s, 1H), 7.52-7.47 (m, 2H), 7.42 (dd, J=7.9, 1.6 Hz, 1H), 7.37-7.32 (m, 2H), 7.19 (d, J=8.3 Hz, 1H), 7.15 (s, 1H), 7.09 (dd, J=7.7, 1.6 Hz, 1H), 6.96 (dd, J=14.8, 2.0 Hz, 2H), 6.89 (dd, J=8.3, 2.5 Hz, 1H), 5.60 (t, J=7.8 Hz, 1H), 4.74 (d, J=9.3 Hz, 1H), 4.63-4.46 (m, 7H), 3.85 (d, J=11.1 Hz, 1H), 3.79 (dd, J=11.1, 3.8 Hz, 1H), 3.61-3.52 (m, 8H), 3.49-3.41 (m, 4H), 3.18-2.95 (m, 8H), 2.90-2.81 (m, 1H), 2.64-2.56 (m, 1H), 2.50 (s, 3H), 2.22 (dd, J=13.3, 7.7 Hz, 1H), 2.13-2.03 (m, 2H), 1.41-1.23 (m, 4H), 1.02 (s, 9H). HRMS m/z [M+H]⁺ calcd for C₅₈H₇₁FN₉O₁₄S⁺ 1168.4820, found 1168.4813.

### Example 311

### Synthesis of LQ141-29

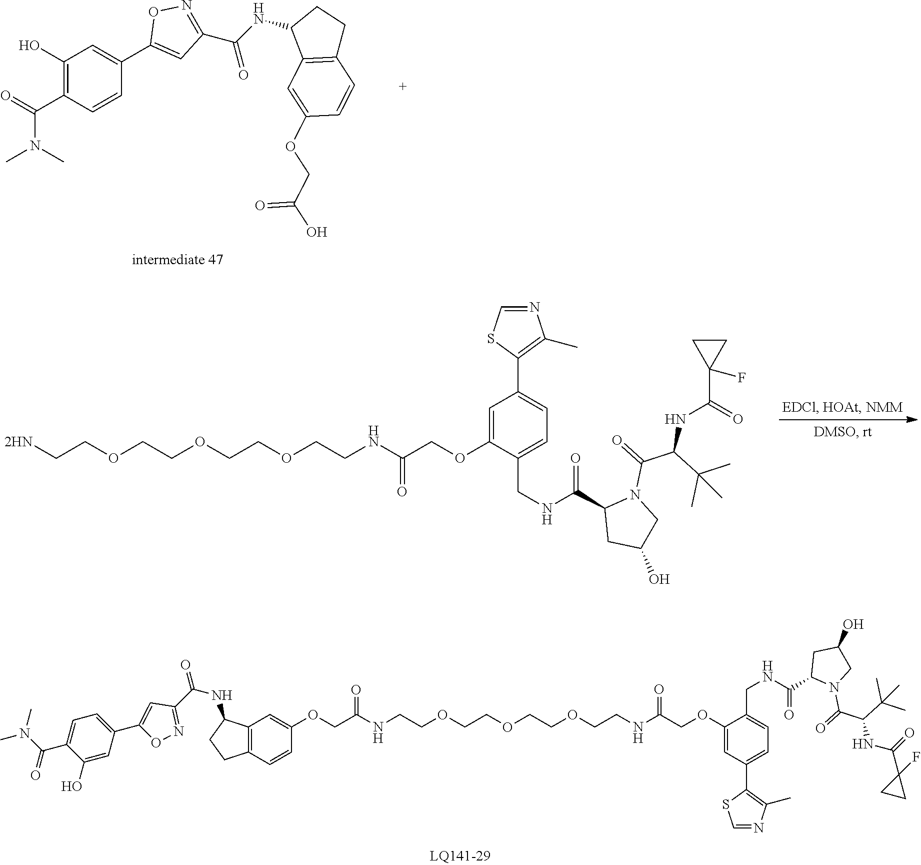

LQ141-29 was synthesized following the standard procedure for preparing LQ126-177 from intermediate 47 (5 mg, 0.01 mmol), (2S,4R)—N-(2-((14-amino-2-oxo-6,9,12-trioxa-3-azatetradecyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (8.8 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-29 was obtained as white solid (7.4 mg, 61%). ¹H NMR (600 MHz, Methanol-d₄) δ 9.10 (s, 1H), 7.53-7.48 (m, 2H), 7.42 (dd, J=7.9, 1.6 Hz, 1H), 7.38-7.32 (m, 2H), 7.20 (d, J=8.3 Hz, 1H), 7.15 (s, 1H), 7.10 (dd, J=7.8, 1.6 Hz, 1H), 6.99 (d, J=1.6 Hz, 1H), 6.95 (d, J=2.4 Hz, 1H), 6.89 (dd, J=8.2, 2.5 Hz, 1H), 5.61 (t, J=7.8 Hz, 1H), 4.75 (d, J=9.2 Hz, 1H), 4.64-4.58 (m, 3H), 4.54-4.46 (m, 4H), 3.85 (d, J=11.0 Hz, 1H), 3.80 (dd, J=11.0, 3.8 Hz, 1H), 3.60-3.51 (m, 12H), 3.49-3.41 (m, 4H), 3.17-2.94 (m, 8H), 2.90-2.82 (m, 1H), 2.64-2.56 (m, 1H), 2.51 (s, 3H), 2.22 (dd, J=13.3, 7.7 Hz, 1H), 2.13-2.04 (m, 2H), 1.41-1.23 (m, 4H), 1.02 (s, 9H). HRMS m/z [M+H]⁺ calcd for C₆₀H₇₅FN₉O₁₅S⁺ 1212.5082, found 1212.5037.

### Example 312

### Synthesis of LQ141-33

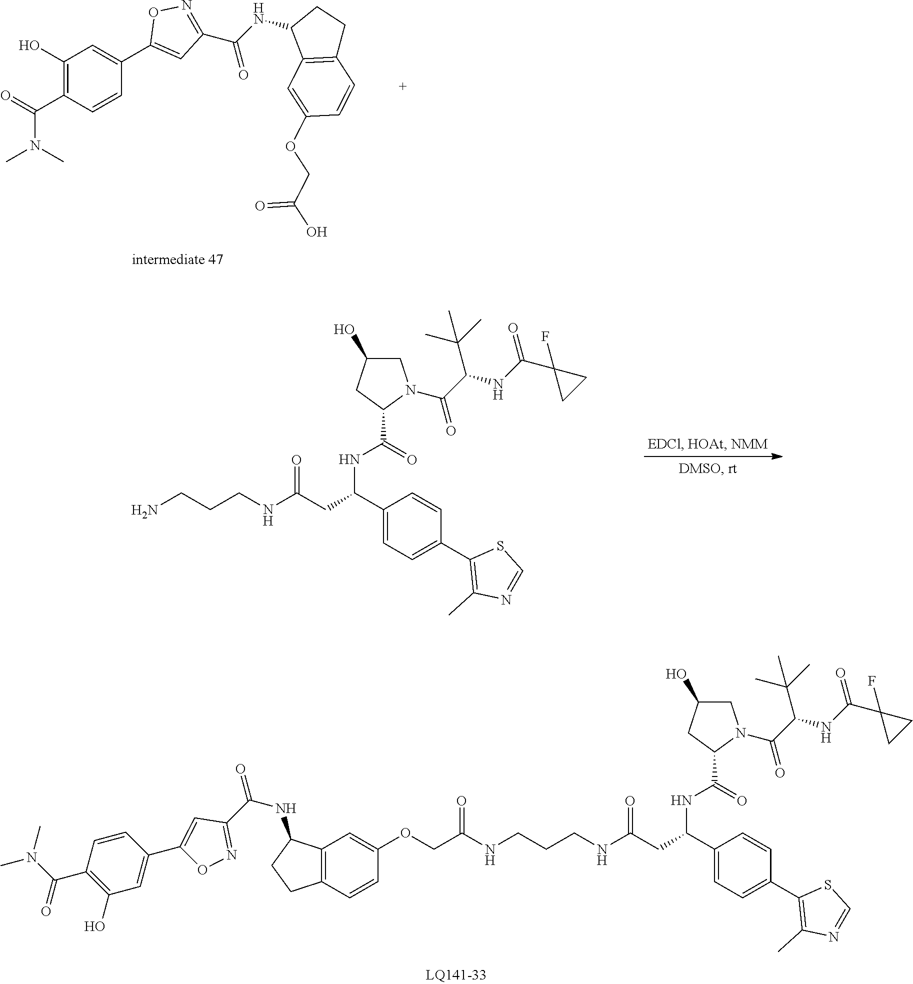

LQ141-33 was synthesized following the standard procedure for preparing LQ126-177 from intermediate 47 (5 mg, 0.01 mmol), (2S,4R)—N—((S)-3-((3-aminopropyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (7.4 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-33 was obtained as white solid (7.2 mg, 67%). ¹H NMR (600 MHz, Methanol-d₄) δ 9.14 (s, 1H), 7.51-7.41 (m, 5H), 7.38-7.32 (m, 2H), 7.21 (d, J=8.3 Hz, 1H), 7.16 (s, 1H), 6.96 (d, J=2.4 Hz, 1H), 6.91 (dd, J=8.3, 2.5 Hz, 1H), 5.62 (t, J=7.9 Hz, 1H), 5.33 (dd, J=8.1, 6.2 Hz, 1H), 4.75 (d, J=8.6 Hz, 1H), 4.64-4.58 (m, 1H), 4.47-4.43 (m, 3H), 3.84 (d, J=11.1 Hz, 1H), 3.77 (dd, J=11.1, 3.8 Hz, 1H), 3.19-2.93 (m, 12H), 2.91-2.82 (m, 2H), 2.75 (dd, J=14.2, 8.2 Hz, 1H), 2.65-2.57 (m, 1H), 2.49 (s, 3H), 2.24-2.17 (m, 1H), 2.15-2.06 (m, 1H), 1.99-1.92 (m, 1H), 1.57-1.51 (m, 2H), 1.41-1.24 (m, 3H), 1.06 (s, 9H). HRMS m/z [M+H]⁺ calcd for C₅₅H₆₅FN₉O₁₁S⁺ 1078.4503, found 1078.4519.

### Example 313

### Synthesis of LQ141-36

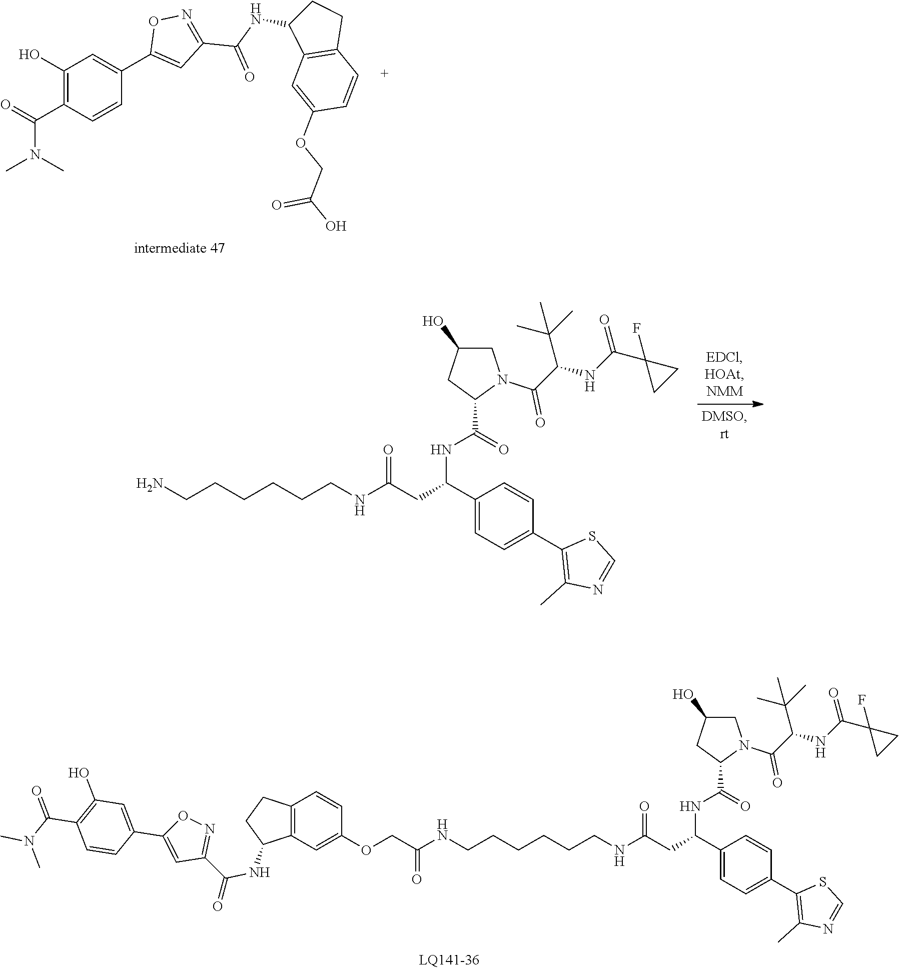

LQ141-36 was synthesized following the standard procedure for preparing LQ126-177 from intermediate 47 (5 mg, 0.01 mmol), (2S,4R)—N—((S)-3-((6-aminohexyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (7.8 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-36 was obtained as white solid (6.7 mg, 60%). ¹H NMR (600 MHz, Methanol-d₄) δ 9.04 (s, 1H), 7.51-7.42 (m, 6H), 7.38-7.34 (m, 2H), 7.20 (d, J=8.3 Hz, 1H), 7.15 (s, 1H), 6.95 (d, J=2.4 Hz, 1H), 6.90 (dd, J=8.3, 2.5 Hz, 1H), 5.62 (t, J=7.9 Hz, 1H), 5.32 (dd, J=8.3, 6.0 Hz, 1H), 4.77-4.73 (m, 1H), 4.60 (dd, J=9.3, 7.6 Hz, 1H), 4.48-4.43 (m, 3H), 3.84 (d, J=10.9 Hz, 1H), 3.78 (dd, J=11.1, 3.8 Hz, 1H), 3.21-2.95 (m, 12H), 2.91-2.82 (m, 2H), 2.76 (dd, J=14.1, 8.3 Hz, 1H), 2.64-2.57 (m, 1H), 2.49 (s, 3H), 2.23-2.18 (m, 1H), 2.13-2.06 (m, 1H), 2.01-1.93 (m, 1H), 1.45-1.26 (m, 7H), 1.23-1.11 (m, 4H), 1.07 (s, 9H). HRMS m/z [M+H]⁺ calcd for C₅₈H₇₁FN₉O₁₁S⁺ 1120.4972, found 1120.4978.

### Example 314

### Synthesis of LQ141-37

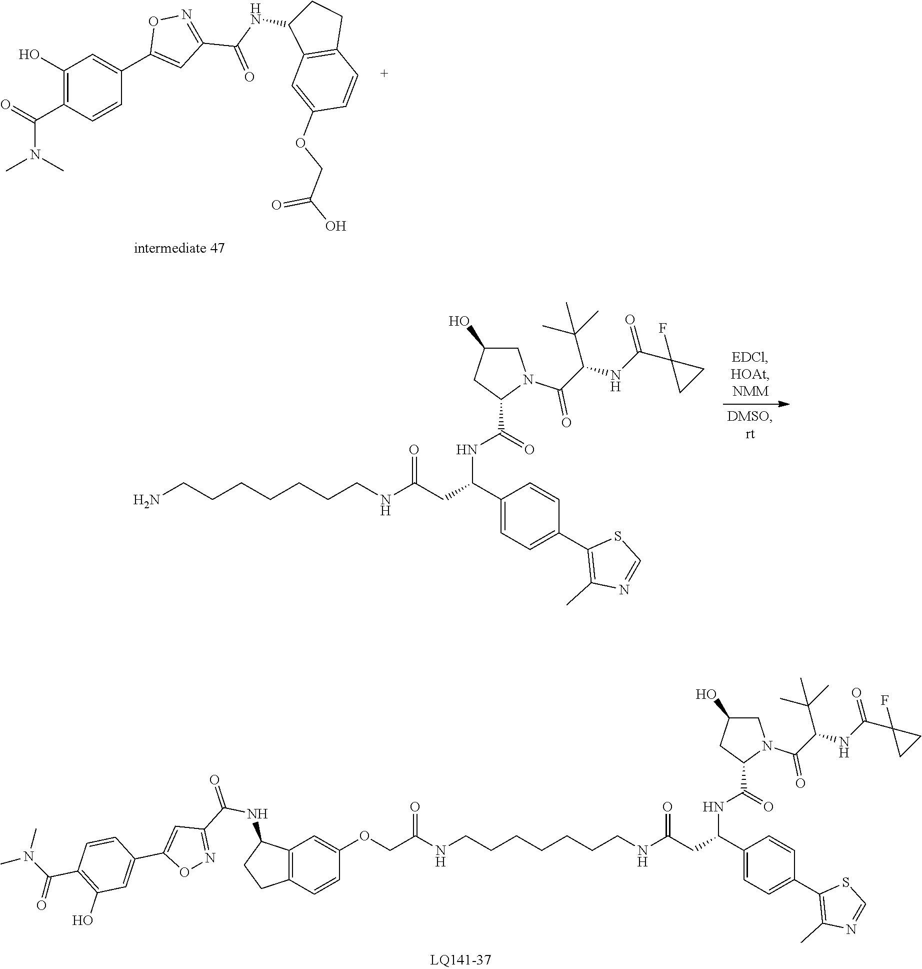

LQ141-37 was synthesized following the standard procedure for preparing LQ126-177 from intermediate 47 (5 mg, 0.01 mmol), (2S,4R)—N—((S)-3-((7-aminoheptyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (8 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-37 was obtained as white solid (7.9 mg, 70%). ¹H NMR (600 MHz, Methanol-d₄) δ 9.03 (s, 1H), 7.52-7.41 (m, 5H), 7.39-7.33 (m, 2H), 7.21 (d, J=8.4 Hz, 1H), 7.15 (s, 1H), 6.96-6.94 (m, 1H), 6.92-6.88 (m, 1H), 5.62 (t, J=7.8 Hz, 1H), 5.32 (dd, J=8.4, 5.9 Hz, 1H), 4.75 (d, J=9.1 Hz, 1H), 4.62-4.58 (m, 1H), 4.49-4.43 (m, 3H), 3.84 (d, J=11.1 Hz, 1H), 3.80-3.75 (m, 1H), 3.21-2.94 (m, 12H), 2.92-2.82 (m, 2H), 2.75 (dd, J=14.0, 8.4 Hz, 1H), 2.65-2.57 (m, 1H), 2.50 (s, 3H), 2.24-2.17 (m, 1H), 2.14-2.05 (m, 1H), 2.00-1.94 (m, 1H), 1.46-1.26 (m, 7H), 1.23-1.15 (m, 4H), 1.14-1.09 (m, 2H), 1.07 (s, 9H). HRMS m/z [M+H]⁺ calcd for C₅₉H₇₃FN₉O₁₁S⁺ 1134.5129, found 1134.5123.

### Example 315

### Synthesis of LQ141-38

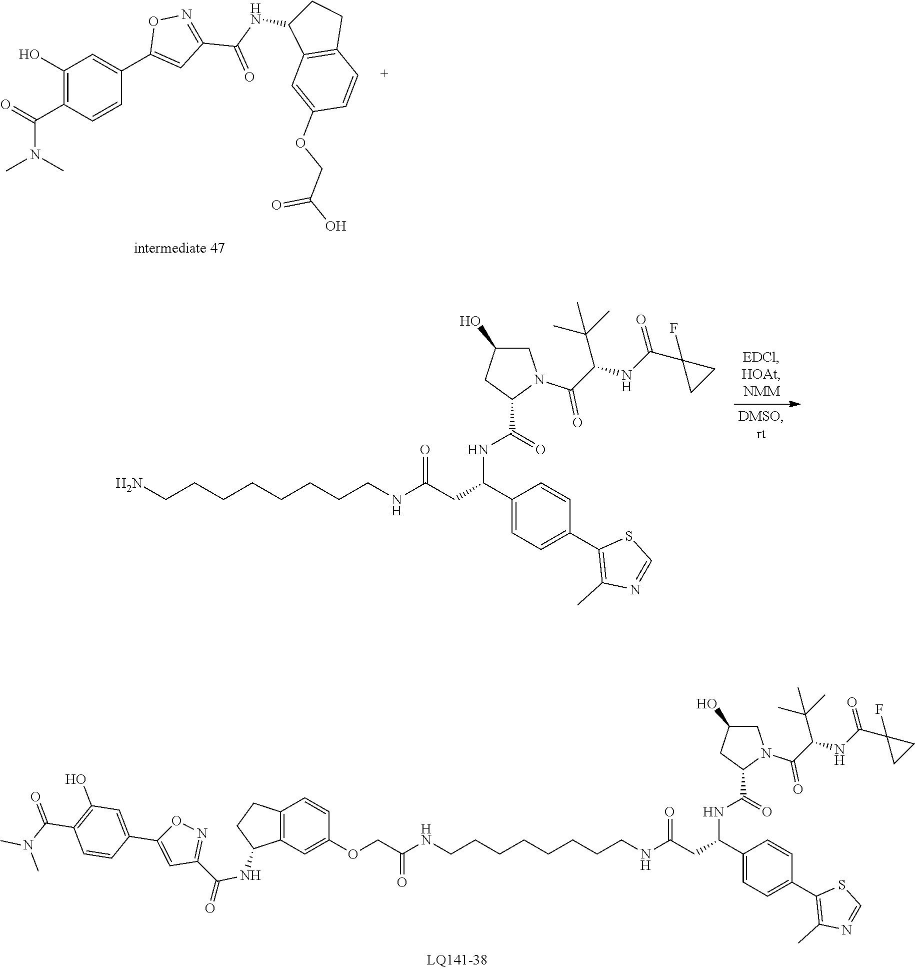

LQ141-38 was synthesized following the standard procedure for preparing LQ126-177 from intermediate 47 (5 mg, 0.01 mmol), (2S,4R)—N—((S)-3-((8-aminooctyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (8.1 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-38 was obtained as white solid (8.6 mg, 73%). ¹H NMR (600 MHz, Methanol-d₄) δ 9.08 (s, 1H), 7.59-7.31 (m, 7H), 7.27-7.13 (m, 2H), 7.02-6.88 (m, 2H), 5.62 (t, J=7.9 Hz, 1H), 5.35-5.30 (m, 1H), 4.75 (d, J=8.9 Hz, 1H), 4.63-4.57 (m, 1H), 4.54-4.43 (m, 3H), 3.84 (d, J=11.1 Hz, 1H), 3.78 (dd, J=11.1, 3.7 Hz, 1H), 3.26-2.94 (m, 12H), 2.93-2.84 (m, 2H), 2.75 (dd, J=13.9, 8.6 Hz, 1H), 2.65-2.58 (m, 1H), 2.50 (s, 3H), 2.24-2.18 (m, 1H), 2.14-2.05 (m, 1H), 2.01-1.94 (m, 1H), 1.47-1.26 (m, 9H), 1.23-1.14 (m, 6H), 1.07 (s, 9H). HRMS m/z [M+H]⁺ calcd for C₆₀H₇₅FN₉O₁₁S⁺ 1148.5285, found 1148.5293.

### Example 316

### Synthesis of LQ141-39

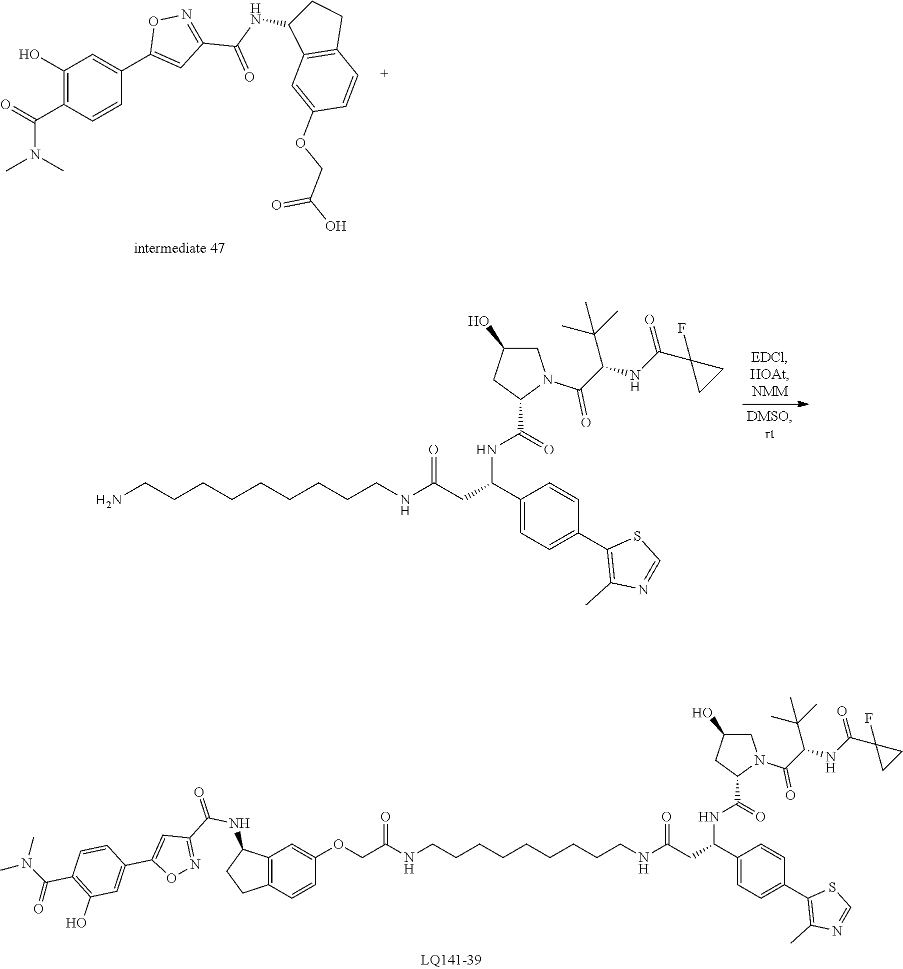

LQ141-39 was synthesized following the standard procedure for preparing LQ126-177 from intermediate 47 (5 mg, 0.01 mmol), (2S,4R)—N—((S)-3-((9-aminononyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (8.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-39 was obtained as white solid (8.1 mg, 70%). ¹H NMR (600 MHz, Methanol-d₄) δ 9.08 (s, 1H), 7.57-7.32 (m, 7H), 7.25-7.13 (m, 2H), 7.00-6.88 (m, 2H), 5.62 (t, J=8.0 Hz, 1H), 5.35-5.30 (m, 1H), 4.75 (d, J=8.9 Hz, 1H), 4.63-4.57 (m, 1H), 4.53-4.45 (m, 3H), 3.85 (d, J=11.1 Hz, 1H), 3.78 (dd, J=11.1, 3.6 Hz, 1H), 3.22 (t, J=7.2 Hz, 2H), 3.19-2.93 (m, 10H), 2.92-2.82 (m, 2H), 2.79-2.72 (m, 1H), 2.64-2.59 (m, 1H), 2.50 (s, 3H), 2.24-2.18 (m, 1H), 2.13-2.05 (m, 1H), 2.01-1.94 (m, 1H), 1.55-1.26 (m, 10H), 1.24-1.11 (m, 7H), 1.08 (s, 9H). HRMS m/z [M+H]⁺ calcd for C₆₁H₇₇FN₉O₁₁S⁺ 1162.5442, found 1162.5441.

### Example 317

### Synthesis of LQ141-42

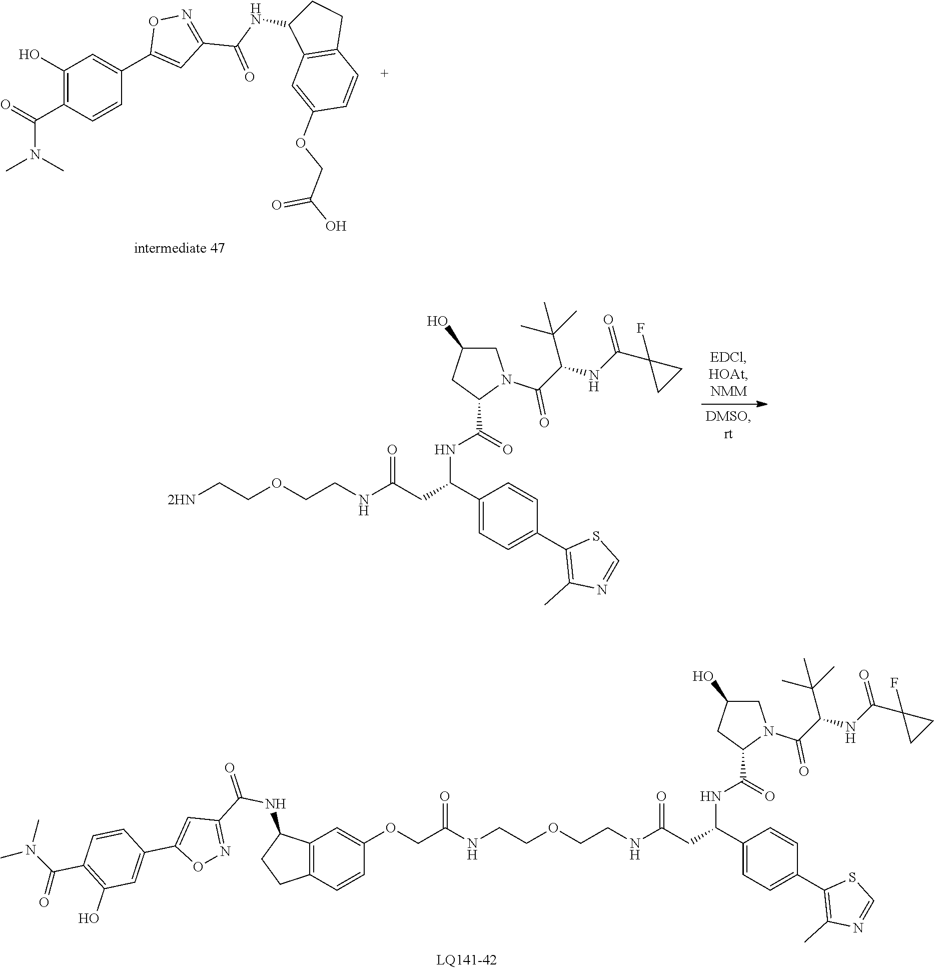

LQ141-42 was synthesized following the standard procedure for preparing LQ126-177 from intermediate 47 (5 mg, 0.01 mmol), (2S,4R)—N—((S)-3-((2-(2-aminoethoxy)ethyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (7.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-42 was obtained as white solid (7.6 mg, 69%). ¹H NMR (600 MHz, Methanol-d₄) δ 9.07 (s, 1H), 7.57-7.30 (m, 7H), 7.22-7.14 (m, 2H), 7.00-6.83 (m, 2H), 5.61 (t, J=7.9 Hz, 1H), 5.36-5.30 (m, 1H), 4.75 (d, J=8.9 Hz, 1H), 4.64-4.57 (m, 1H), 4.53-4.41 (m, 3H), 3.84 (d, J=11.1 Hz, 1H), 3.77 (dd, J=11.1, 3.9 Hz, 1H), 3.52-3.35 (m, 5H), 3.29-3.22 (m, 2H), 3.19-2.92 (m, 8H), 2.90-2.82 (m, 2H), 2.75 (dd, J=14.2, 8.0 Hz, 1H), 2.64-2.56 (m, 1H), 2.49 (s, 3H), 2.24-2.18 (m, 1H), 2.14-2.04 (m, 1H), 2.02-1.90 (m, 1H), 1.45-1.22 (m, 4H), 1.06 (s, 9H). HRMS m/z [M+H]⁺ calcd for C₅₆H₆₇FN₉O₁₂S⁺ 1108.4608, found 1108.4601.

### Example 318

### Synthesis of LQ141-43

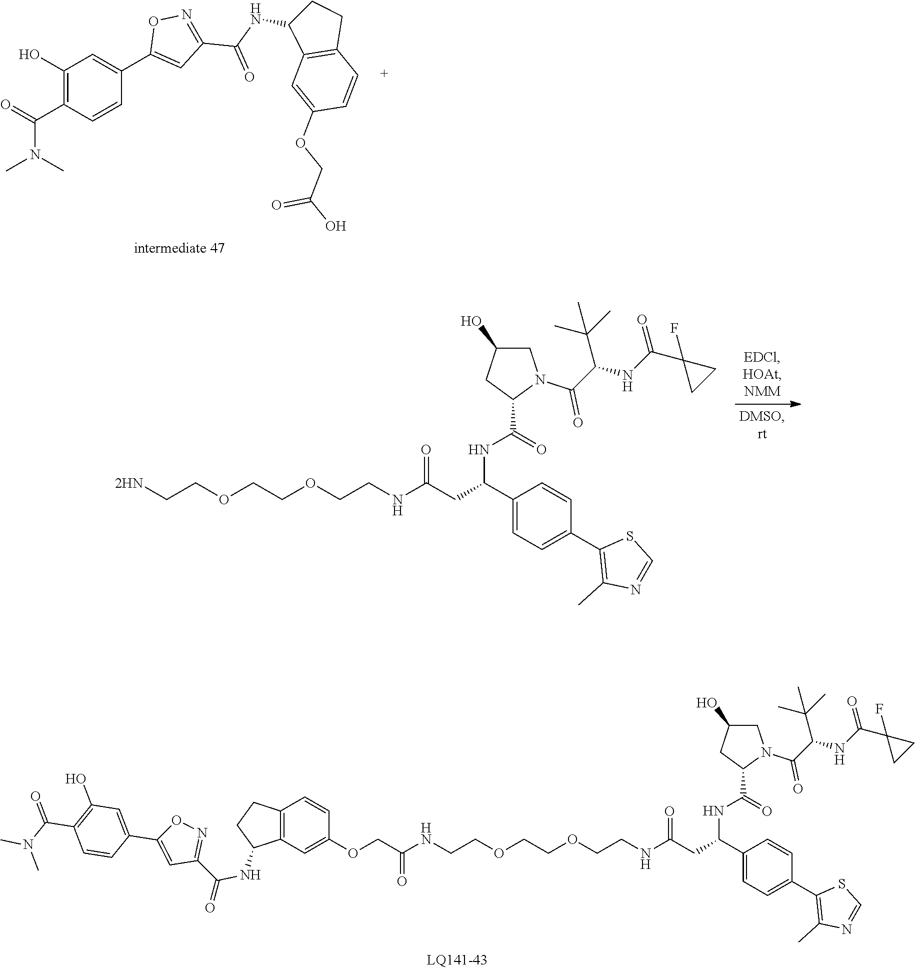

LQ141-43 was synthesized following the standard procedure for preparing LQ126-177 from intermediate 47 (5 mg, 0.01 mmol), (2S,4R)—N—((S)-3-((2-(2-(2-aminoethoxy)ethoxy)ethyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (8.2 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-43 was obtained as white solid (7.5 mg, 65%). ¹H NMR (600 MHz, Methanol-d₄) δ 9.14 (s, 1H), 7.52-7.41 (m, 5H), 7.40-7.33 (m, 2H), 7.25-7.15 (m, 2H), 7.01-6.85 (m, 2H), 5.67-5.59 (m, 1H), 5.35-5.30 (m, 1H), 4.75 (d, J=8.5 Hz, 1H), 4.63-4.58 (m, 1H), 4.53-4.42 (m, 3H), 3.84 (d, J=10.7 Hz, 1H), 3.76 (dd, J=11.2, 3.9 Hz, 1H), 3.63-3.36 (m, 9H), 3.31-3.26 (m, 2H), 3.21-2.96 (m, 8H), 2.92-2.83 (m, 2H), 2.79-2.71 (m, 1H), 2.66-2.58 (m, 1H), 2.50 (s, 3H), 2.27-2.19 (m, 1H), 2.16-2.05 (m, 1H), 2.03-1.93 (m, 1H), 1.44-1.26 (m, 4H), 1.07 (s, 9H). HRMS m/z [M+H]⁺ calcd for C₅₈H₇₁FN₉O₁₃S⁺ 1152.4871, found 1152.4874.

### Example 319

### Synthesis of LQ141-44

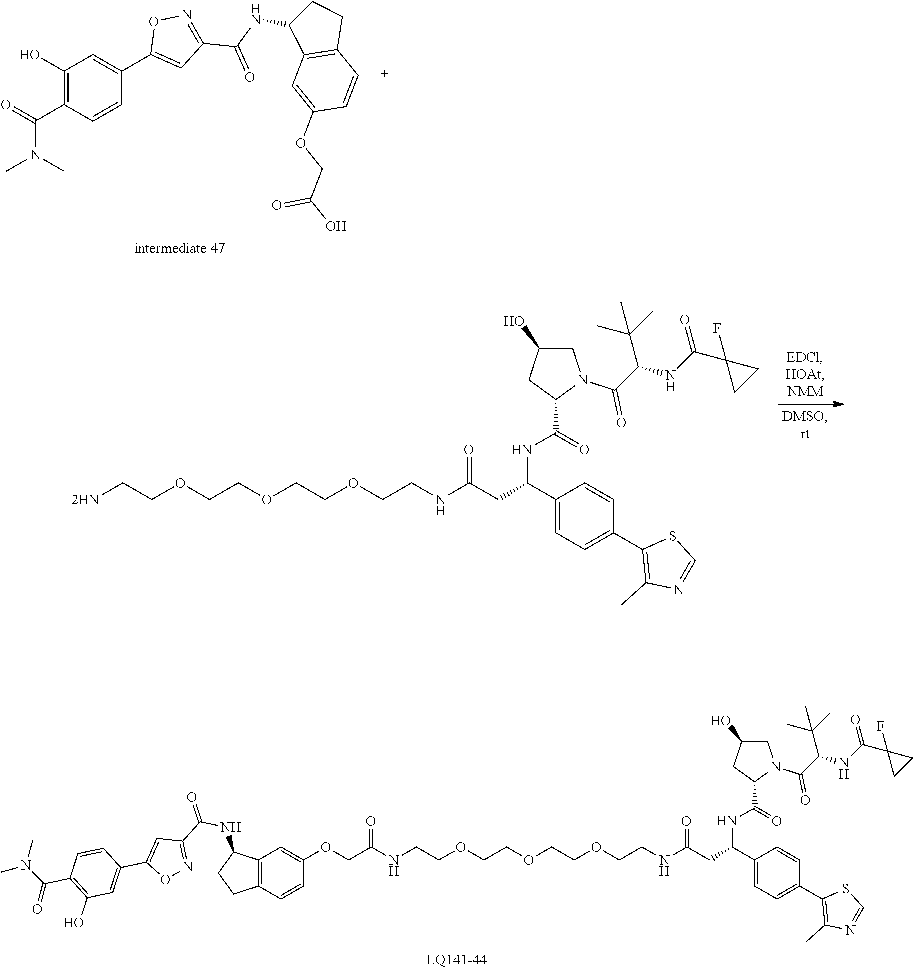

LQ141-44 was synthesized following the standard procedure for preparing LQ126-177 from intermediate 47 (5 mg, 0.01 mmol), (2S,4R)—N—((S)-1-amino-15-(4-(4-methylthiazol-5-yl)phenyl)-13-oxo-3,6,9-trioxa-12-azapentadecan-15-yl)-1-((S)-2-(1-fluorocyclopropane-1-carboxamido)-3,3-dimethylbutanoyl)-4-hydroxypyrrolidine-2-carboxamide (8.6 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-44 was obtained as white solid (7.2 mg, 60%). ¹H NMR (600 MHz, Methanol-d₄) δ 8.95 (s, 1H), 7.57-7.31 (m, 7H), 7.25-7.14 (m, 2H), 6.99-6.86 (m, 2H), 5.62 (t, J=7.8 Hz, 1H), 5.36-5.30 (m, 1H), 4.78-4.72 (m, 1H), 4.60 (t, J=8.6 Hz, 1H), 4.53-4.43 (m, 3H), 3.84 (d, J=11.1 Hz, 1H), 3.77 (dd, J=11.2, 3.7 Hz, 1H), 3.68-3.39 (m, 13H), 3.31-3.23 (m, 2H), 3.19-2.96 (m, 8H), 2.91-2.82 (m, 2H), 2.80-2.73 (m, 1H), 2.65-2.57 (m, 1H), 2.48 (s, 3H), 2.25-2.18 (m, 1H), 2.13-2.06 (m, 1H), 2.01-1.93 (m, 1H), 1.45-1.23 (m, 4H), 1.07 (s, 9H). HRMS m/z [M+H]⁺ calcd for C₆₀H₇₅FN₉O₁₄S⁺ 1196.5133, found 1196.5125.

### Example 320

### Synthesis of LQ141-45

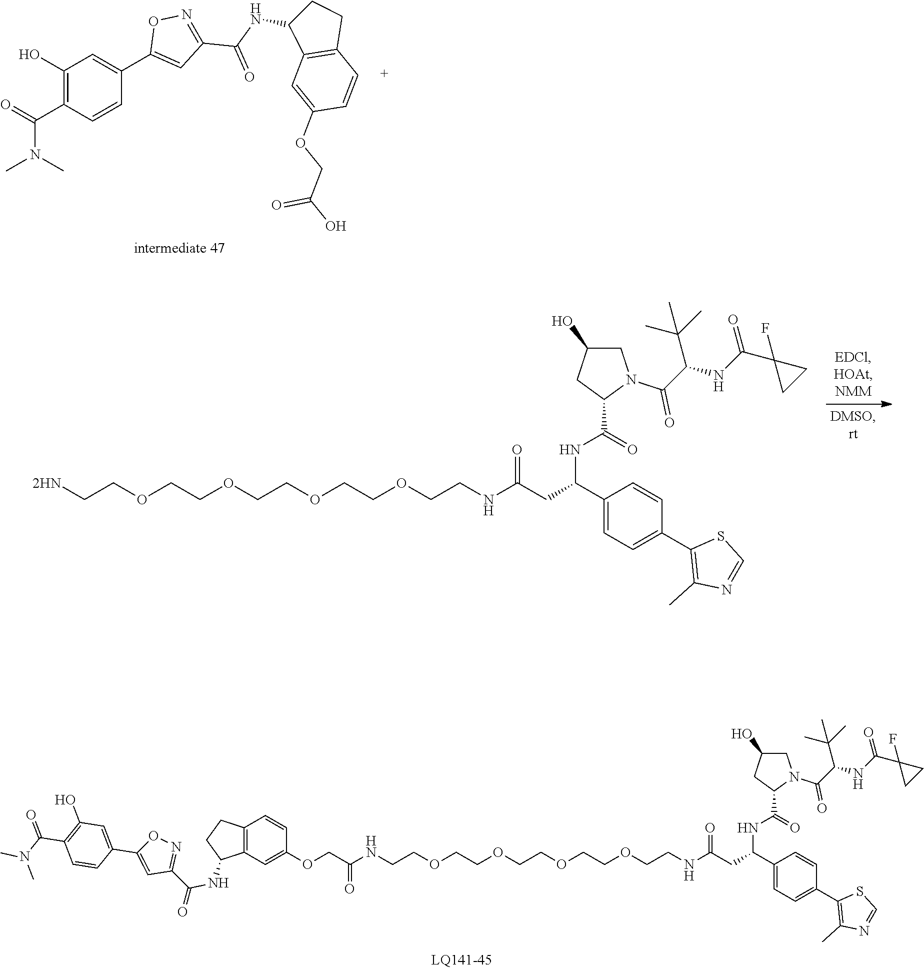

LQ141-45 was synthesized following the standard procedure for preparing LQ126-177 from intermediate 47 (5 mg, 0.01 mmol), (2S,4R)—N—((S)-1-amino-18-(4-(4-methylthiazol-5-yl)phenyl)-16-oxo-3,6,9,12-tetraoxa-15-azaoctadecan-18-yl)-1-((S)-2-(1-fluorocyclopropane-1-carboxamido)-3,3-dimethylbutanoyl)-4-hydroxypyrrolidine-2-carboxamide (9 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-45 was obtained as white solid (9 mg, 73%). ¹H NMR (600 MHz, Methanol-d₄) δ 9.01 (s, 1H), 7.60-7.30 (m, 7H), 7.25-7.13 (m, 2H), 6.99-6.86 (m, 2H), 5.62 (t, J=7.8 Hz, 1H), 5.37-5.31 (m, 1H), 4.75 (d, J=9.0 Hz, 1H), 4.60 (t, J=8.6 Hz, 1H), 4.54-4.42 (m, 3H), 3.84 (d, J=11.2 Hz, 1H), 3.77 (dd, J=11.1, 3.8 Hz, 1H), 3.71-3.34 (m, 17H), 3.31-3.22 (m, 2H), 3.18-2.93 (m, 8H), 2.91-2.82 (m, 2H), 2.81-2.74 (m, 1H), 2.64-2.57 (m, 1H), 2.49 (s, 3H), 2.25-2.18 (m, 1H), 2.14-2.05 (m, 1H), 2.01-1.93 (m, 1H), 1.43-1.24 (m, 4H), 1.08 (s, 9H). HRMS m/z [M+H]⁺ calcd for C₆₂H₇₉FN₉O₁₅S⁺ 1240.5395, found 1240.5403.

### Example 321

### Synthesis of LQ141-46

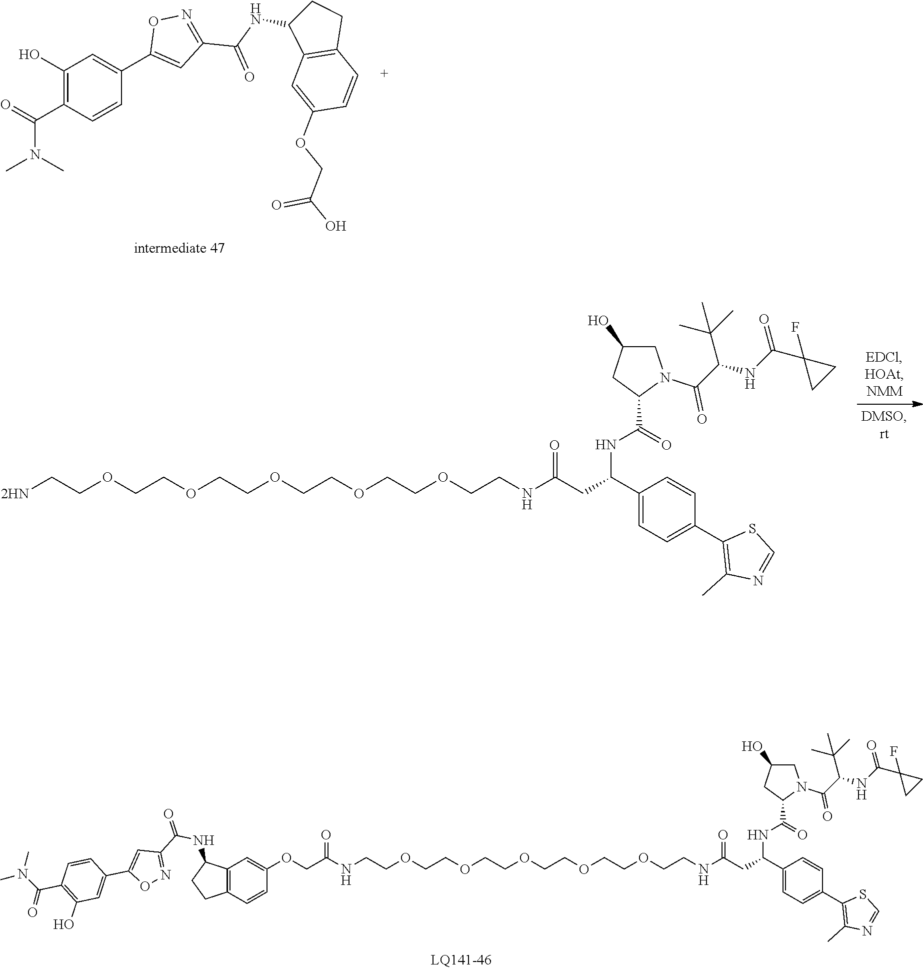

LQ141-46 was synthesized following the standard procedure for preparing LQ126-177 from intermediate 47 (5 mg, 0.01 mmol), (2S,4R)—N—((S)-1-amino-21-(4-(4-methylthiazol-5-yl)phenyl)-19-oxo-3,6,9,12,15-pentaoxa-18-azahenicosan-21-yl)-1-((S)-2-(1-fluorocyclopropane-1-carboxamido)-3,3-dimethylbutanoyl)-4-hydroxypyrrolidine-2-carboxamide (9.5 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-46 was obtained as white solid (8.5 mg, 66%). ¹H NMR (600 MHz, Methanol-d₄) δ 9.08 (s, 1H), 7.63-7.30 (m, 7H), 7.26-7.12 (m, 2H), 6.98-6.85 (m, 2H), 5.63 (t, J=8.5 Hz, 1H), 5.44-5.31 (m, 1H), 4.75 (d, J=9.0 Hz, 1H), 4.63-4.57 (m, 1H), 4.56-4.42 (m, 3H), 3.84 (d, J=11.3 Hz, 1H), 3.80-3.75 (m, 1H), 3.70-3.41 (m, 21H), 3.34-3.22 (m, 2H), 3.20-2.94 (m, 8H), 2.90-2.83 (m, 2H), 2.81-2.74 (m, 1H), 2.65-2.56 (m, 1H), 2.50 (s, 3H), 2.25-2.20 (m, 1H), 2.13-2.04 (m, 1H), 2.03-1.92 (m, 1H), 1.49-1.23 (m, 4H), 1.06 (s, 9H). HRMS m/z [M+H]⁺ calcd for C₆₄H₈₃FN₉O₁₆S⁺ 1284.5657, found 1284.5608.

### Example 322

### Synthesis of LQ141-47

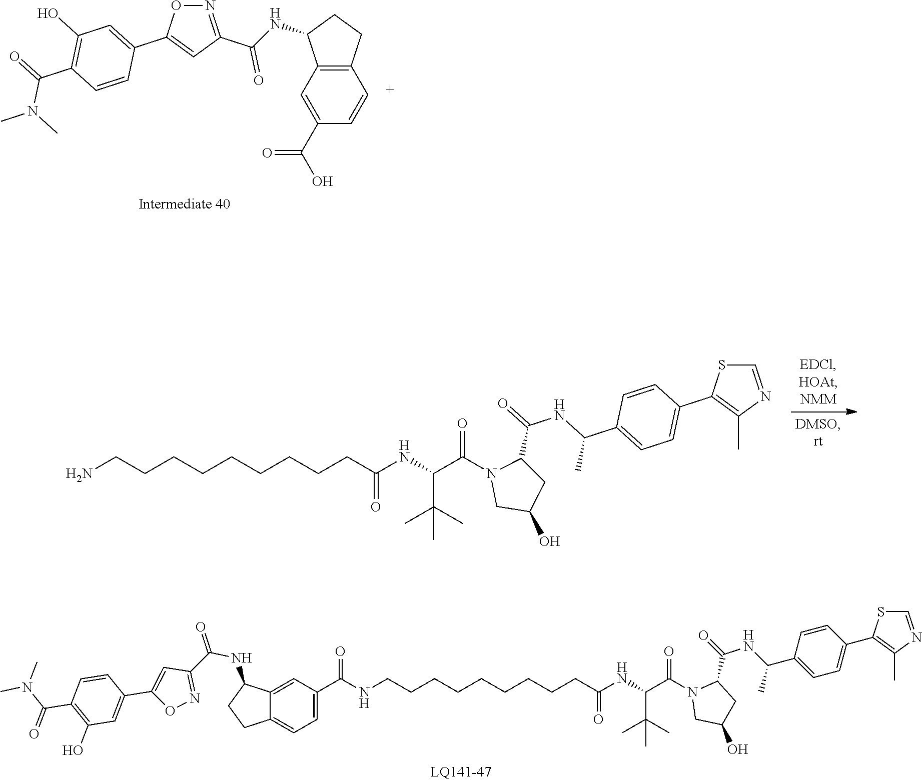

LQ141-47 was synthesized following the standard procedure for preparing LQ108-58 from intermediate 40 (5 mg, 0.01 mmol), (2S,4R)-1-((S)-2-(10-aminodecanamido)-3,3-dimethylbutanoyl)-4-hydroxy-N—((S)-1-(4-(4-methylthiazol-5-yl)phenyl)ethyl)pyrrolidine-2-carboxamide (7.3 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-47 was obtained as white solid (6.5 mg, 63%). ¹H NMR (600 MHz, Methanol-d₄) δ 9.18 (s, 1H), 7.79-7.70 (m, 2H), 7.53-7.40 (m, 5H), 7.40-7.32 (m, 3H), 7.15 (d, J=3.2 Hz, 1H), 5.69 (t, J=7.7 Hz, 1H), 4.99 (d, J=7.0 Hz, 1H), 4.65-4.60 (m, 1H), 4.57 (t, J=8.2 Hz, 1H), 4.46-4.39 (m, 1H), 3.98 (d, J=2.8 Hz, 1H), 3.87 (d, J=10.8 Hz, 1H), 3.79-3.69 (m, 1H), 3.37-3.29 (m, 6H), 3.19-3.05 (m, 4H), 3.05-2.91 (m, 4H), 2.69-2.60 (m, 1H), 2.51 (s, 3H), 2.34-2.07 (m, 5H), 1.95 (dd, J=8.8, 4.4 Hz, 1H), 1.66-1.54 (m, 5H), 1.52-1.48 (m, 2H), 1.41-1.26 (m, 10H), 1.03 (s, 9H). HRMS m/z [M+H]⁺ calcd for C₅₆H₇₁N₈O₉S⁺ 1031.5059, found 1031.5058.

### Example 323

### Synthesis of LQ141-48

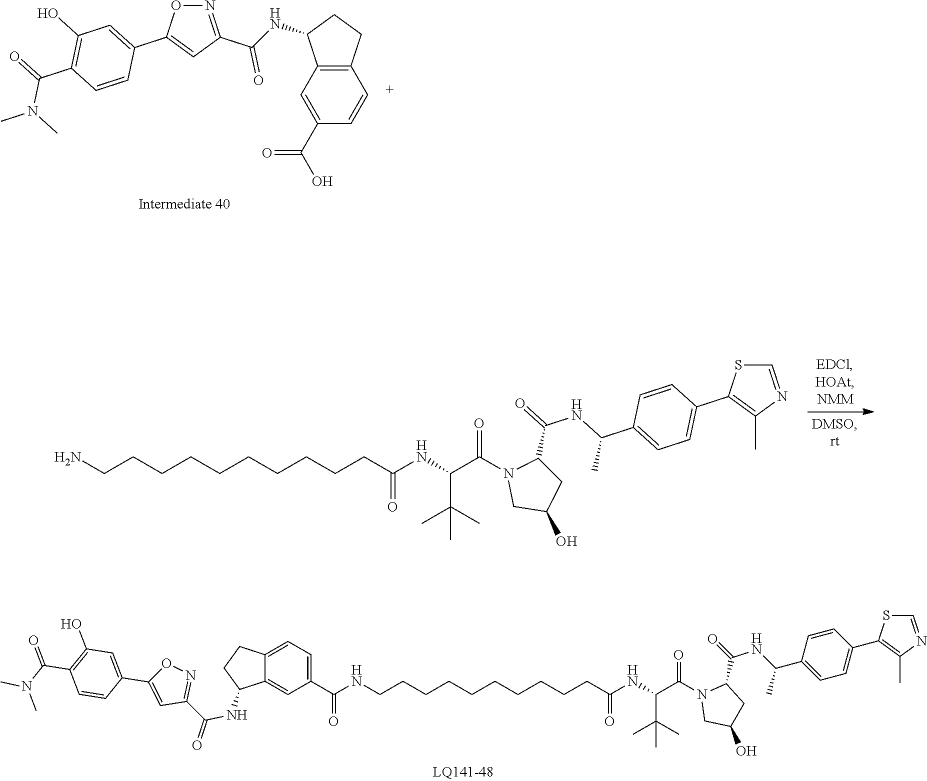

LQ141-48 was synthesized following the standard procedure for preparing LQ108-58 from intermediate 40 (5 mg, 0.01 mmol), (2S,4R)-1-((S)-2-(11-aminoundecanamido)-3,3-dimethylbutanoyl)-4-hydroxy-N—((S)-1-(4-(4-methylthiazol-5-yl)phenyl)ethyl)pyrrolidine-2-carboxamide (7.4 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-48 was obtained as white solid (6.1 mg, 58%). ¹H NMR (600 MHz, Methanol-d₄) δ 9.22 (s, 1H), 7.75 (d, J=5.0 Hz, 1H), 7.72 (dd, J=7.9, 1.7 Hz, 1H), 7.50-7.30 (m, 8H), 7.14 (s, 1H), 5.68 (t, J=7.9 Hz, 1H), 4.99 (q, J=7.0 Hz, 1H), 4.60 (s, 1H), 4.56 (t, J=8.3 Hz, 1H), 4.41 (dt, J=4.3, 2.2 Hz, 1H), 3.97 (s, 1H), 3.86 (dt, J=11.2, 1.8 Hz, 1H), 3.73 (dd, J=11.0, 4.0 Hz, 1H), 3.35-3.30 (m, 6H), 3.14-3.02 (m, 4H), 2.96 (dt, J=16.5, 8.4 Hz, 4H), 2.64 (m, 1H), 2.50 (s, 3H), 2.31-2.05 (m, 5H), 1.93 (m, 1H), 1.63-1.52 (m, 4H), 1.49 (m, 3H), 1.31 (m, 12H), 1.02 (s, 9H). HRMS m/z [M+H]⁺ calcd for C₅₇H₇₃N₈O₉S⁺ 1045.5216, found 1045.5211.

### Example 324

### Synthesis of LQ141-49

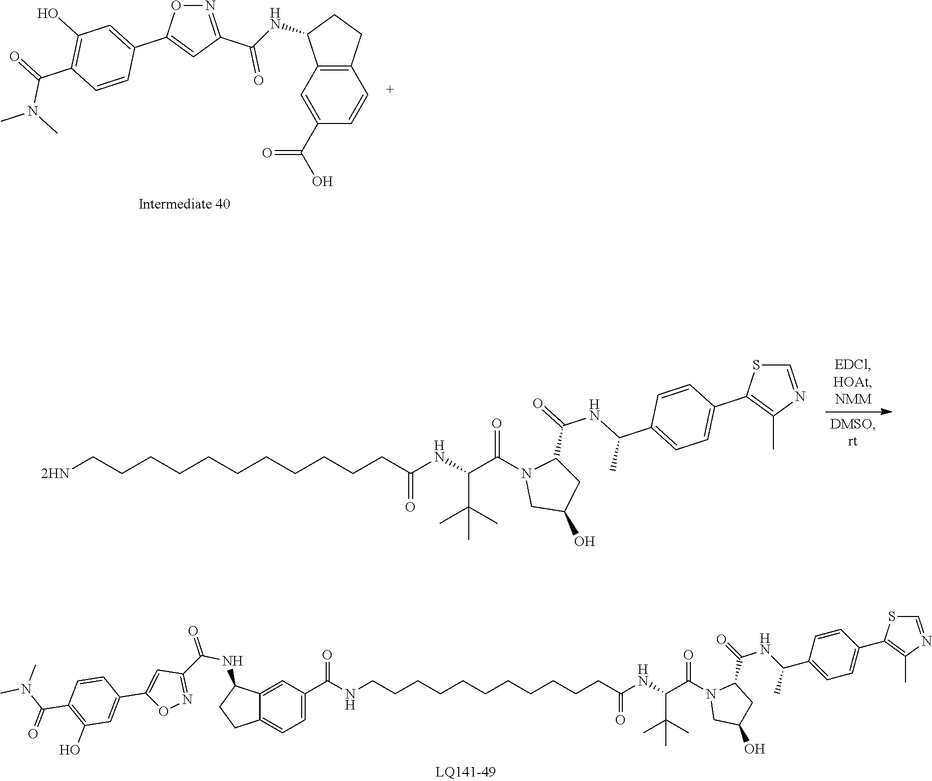

LQ141-49 was synthesized following the standard procedure for preparing LQ108-58 from intermediate 40 (5 mg, 0.01 mmol), (2S,4R)-1-((S)-2-(12-aminododecanamido)-3,3-dimethylbutanoyl)-4-hydroxy-N—((S)-1-(4-(4-methylthiazol-5-yl)phenyl)ethyl)pyrrolidine-2-carboxamide (7.6 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-49 was obtained as white solid (7 mg, 66%). ¹H NMR (600 MHz, Methanol-d₄) δ 9.21 (s, 1H), 7.85-7.66 (m, 2H), 7.60-7.25 (m, 8H), 7.14 (p, J=5.0 Hz, 1H), 5.78-5.62 (m, 1H), 5.08-5.00 (m, 1H), 4.69-4.51 (m, 2H), 4.42 (s, 1H), 3.97 (t, J=3.2 Hz, 1H), 3.87 (d, J=11.0 Hz, 1H), 3.78-3.68 (m, 1H), 3.47-3.23 (m, 6H), 3.21-2.87 (m, 8H), 2.75-2.43 (m, 5H), 2.39-1.87 (m, 6H), 1.73-1.45 (m, 7H), 1.30 (m, 13H), 1.02 (s, 9H). HRMS m/z [M+H]⁺ calcd for C₅₈H₇₅N₈O₉S⁺ 1059.5372, found 1059.5377.

### Example 325

### Synthesis of LQ141-52

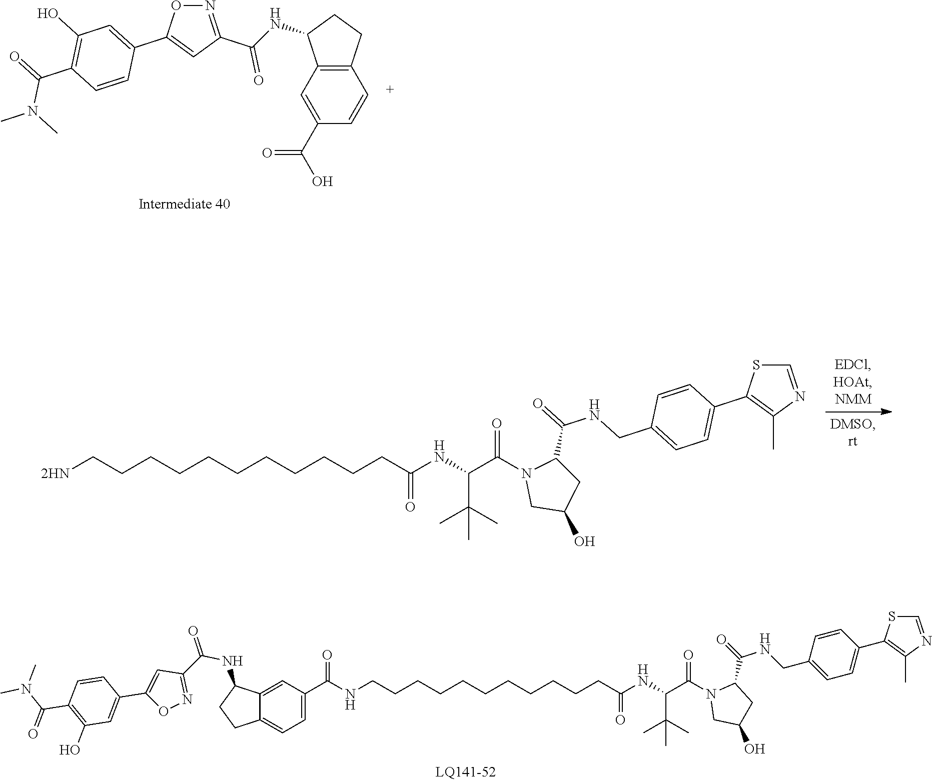

LQ141-52 was synthesized following the standard procedure for preparing LQ108-58 from intermediate 40 (5 mg, 0.01 mmol), (2S,4R)-1-((S)-2-(12-aminododecanamido)-3,3-dimethylbutanoyl)-4-hydroxy-N-(4-(4-methylthiazol-5-yl)benzyl)pyrrolidine-2-carboxamide (7.4 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-52 was obtained as white solid (6.3 mg, 60%). ¹H NMR (600 MHz, Methanol-d₄) δ 9.15 (s, 1H), 7.76 (s, 1H), 7.72 (dd, J=7.9, 1.7 Hz, 1H), 7.49 (d, J=8.0 Hz, 2H), 7.46-7.39 (m, 3H), 7.39-7.30 (m, 3H), 7.15 (s, 1H), 5.68 (t, J=7.9 Hz, 1H), 4.63 (s, 1H), 4.58 (d, J=8.5 Hz, 1H), 4.55 (d, J=15.2 Hz, 1H), 4.49 (m, 1H), 4.36 (d, J=15.5 Hz, 1H), 3.98 (s, 2H), 3.90 (d, J=11.0 Hz, 1H), 3.80 (dd, J=10.9, 3.9 Hz, 1H), 3.33 (m, 6H), 3.12 (m, 4H), 2.97 (m, 4H), 2.68-2.61 (m, 1H), 2.50 (s, 3H), 2.32-2.17 (m, 3H), 2.17-2.05 (m, 2H), 1.59 (m, 4H), 1.47-1.20 (m, 14H), 1.03 (s, 9H). HRMS m/z [M+H]⁺ calcd for C₅₇H₇₃N₈O₉S⁺ 1045.5216, found 1284.5206.

### Example 326

### Synthesis of LQ141-57

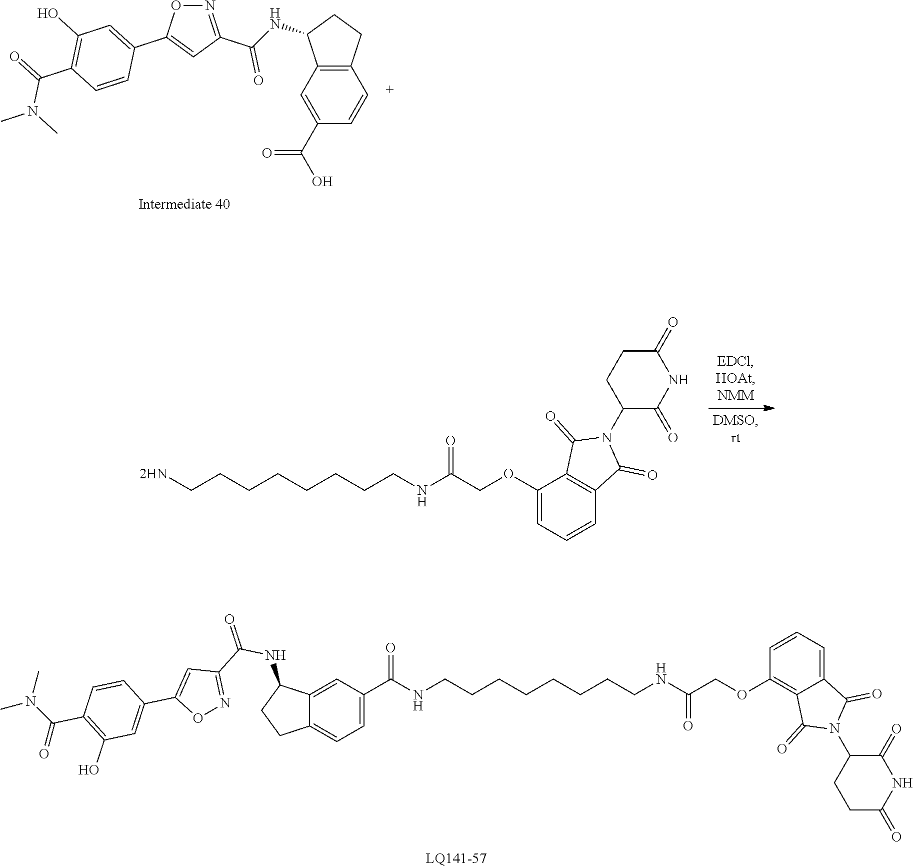

LQ141-57 was synthesized following the standard procedure for preparing LQ108-58 from intermediate 40 (5 mg, 0.01 mmol), N-(8-aminooctyl)-2-((2-(2,6-dioxopiperidin-3-yl)-1,3-dioxoisoindolin-4-yl)oxy)acetamide (5.7 mg, 0.01 mmol, 1.0 equiv), EDCI (2.9 mg, 0.015 mmol, 1.5 equiv), HOAt (2.1 mg, 0.015 mmol, 1.5 equiv), and NMM (3.1 mg, 0.03 mmol, 3.0 equiv) in DMSO (1 mL). LQ141-57 was obtained as white solid (5.3 mg, 61%). ¹H NMR (600 MHz, Methanol-d₄) δ 7.87-7.65 (m, 3H), 7.51 (d, J=7.0 Hz, 1H), 7.38 (m, 5H), 7.20-7.12 (m, 1H), 5.69 (t, J=7.9 Hz, 1H), 5.21-5.10 (m, 1H), 4.79-4.64 (m, 2H), 3.43-3.28 (m, 6H), 3.19-2.93 (m, 8H), 2.93-2.55 (m, 5H), 2.26-2.08 (m, 3H), 1.57 (m, 4H), 1.32 (m, 9H). HRMS m/z [M+H]⁺ calcd for C₄₆H₅₀N₇O₁₁⁺ 876.3563, found 876.3553.

Certain compounds disclosed herein have the structures shown in Table 1.

TABLE 1

| Ex- | Com- | | |
|---|---|---|---|
| am- | pound | | |
| ples | code | Structure | Chemical Name |
| 2 | LQ076- 46 | 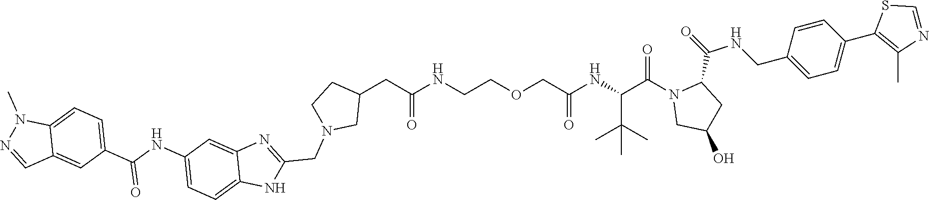 [SMILES: Cc1ncsc1-c1ccc(CNC(=O)[C@@H]2C[C@@H](O)CN2C(=O)[C@@H](NC(=O)COCCNC(=O)CC2CCN(Cc3nc4cc(NC(=O)c5ccc6c(cnn6C)c5)ccc4[nH]3)C2)C(C)(C)C)cc1] | N-(2-((3-(2-((2-(2-(((S)-1-((2S,4R)- 4-hydroxy-4-((2-(4-methylthiazol- 5-yl)benzyl)carbamoyl)pyrrolidin- 1-yl)-3,3-dimethyl-1-oxobutan-2- yl)amino)-2- oxoethoxy)ethyl)amino)-2- oxoethyl)pyrrolidin-1-yl)methyl)- 1H-benzo[d]imidazol-5-yl)-1- methyl-1H-indazole-5-carboxamide |
| 3 | LQ076- 47 | 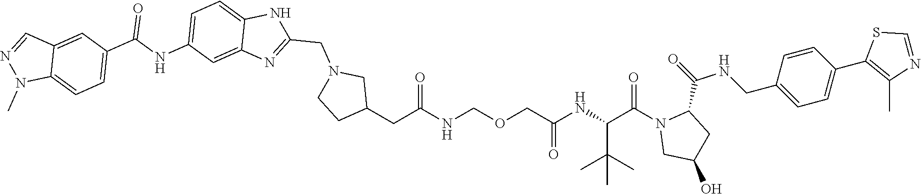 [SMILES: Cc1ncsc1-c1ccc(CNC(=O)[C@@H]2C[C@@H](O)CN2C(=O)[C@@H](NC(=O)COCNC(=O)CC2CCN(Cc3nc4cc(NC(=O)c5ccc6c(cnn6C)c5)ccc4[nH]3)C2)C(C)(C)C)cc1] | N-(2-((3-(2-((2-(3-(((S)-1-((2S,4R)- 4-hydroxy-2-((4-(4-methylthiazol- 5-yl)benzyl)carbamoyl)pyrrolidin- 1-yl)-3,3-dimethyl-1-oxobutan-2- yl)amino)-3- oxopropoxy)ethyl)amino)-2- oxoethyl)pyrrolidin-1-yl)methyl)- 1H-benzo[d]imidazol-5-yl)-1- methyl-1H-indazole-5- carboxamide |
| 4 | LQ076- 48 | 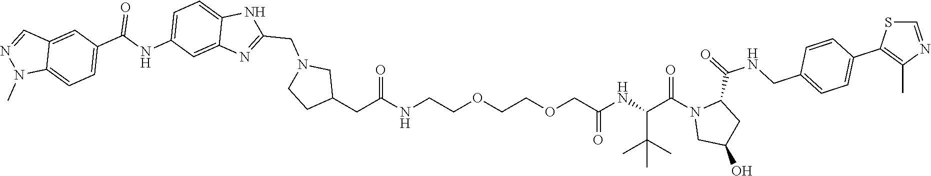 [SMILES: Cc1ncsc1-c1ccc(CNC(=O)[C@@H]2C[C@@H](O)CN2C(=O)[C@@H](NC(=O)COCCOCCNC(=O)CC2CCN(Cc3nc4cc(NC(=O)c5ccc6c(cnn6C)c5)ccc4[nH]3)C2)C(C)(C)C)cc1] | N-(2-((3-((S)-13-((2S,4R)-4- hydroxy-2-((4-(4-methylthiazol-5- yl)benzyl)carbamoyl)pyrrolidine-1- carbonyl)-14,14-dimethyl-2,11- dioxo-6,9-dioxa-3,12- diazapentadecyl)pyrrolidin-1- yl)methyl)-1H-benzo[d]imidazol-5- yl)-1-methyl-1H-indazole-5- carboxamide |
| 5 | LQ076- 49 | 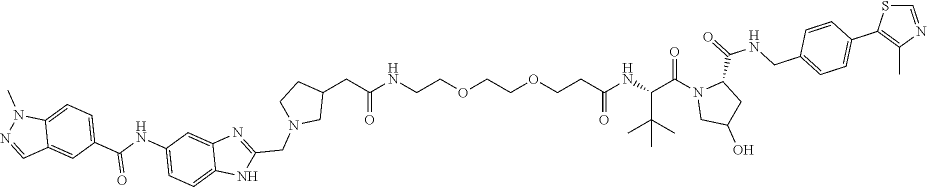 [SMILES: Cc1ncsc1-c1ccc(CNC(=O)[C@@H]2CC(O)CN2C(=O)[C@@H](NC(=O)CCOCCOCCNC(=O)CC2CCN(Cc3nc4cc(NC(=O)c5ccc6c(cnn6C)c5)ccc4[nH]3)C2)C(C)(C)C)cc1] | N-(2-((3-((S)-14-((2S,4R)-4- hydroxy-2-((4-(4-methylthiazol-5- yl)benzyl)carbamoyl)pyrrolidine-1- carbonyl)-15,15-dimethyl-2,12- dioxo-6,9-dioxa-3,13- diazahexadecyl)pyrrolidin-1- yl)methyl)-1H-benzo[d]imidazol-5- yl)-1-mehtyl-1H-indazole-5- carboxamide |
| 6 | LQ076- 50 | 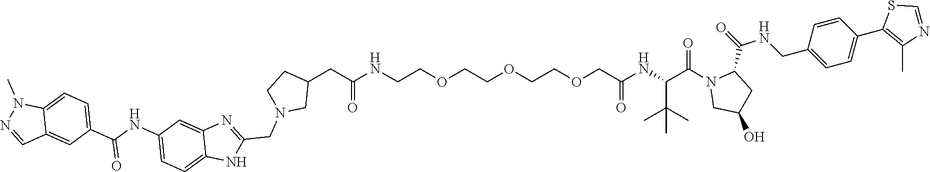 [SMILES: Cc1ncsc1-c1ccc(CNC(=O)[C@@H]2C[C@@H](O)CN2C(=O)[C@@H](NC(=O)COCCOCCOCCNC(=O)CC2CCN(Cc3nc4cc(NC(=O)c5ccc6c(cnn6C)c5)ccc4[nH]3)C2)C(C)(C)C)cc1] | (N-(2-((3-((S)-16-((2S,4R)-4- hydroxy-2-((4-(4-methylthiazol-5- yl)benzyl)carbamoyl)pyrrolidine-1- carbonyl)-17,17-dimethyl-2,14- dioxo-6,9,12-trioxa-3,15- diazaoctadecyl)pyrrolidin-1- yl)methyl)-1H-benzo[d]imidazol-5- yl)-1-methyl-1H-indazole-5- carboxamide |
| 7 | LQ076- 51 | 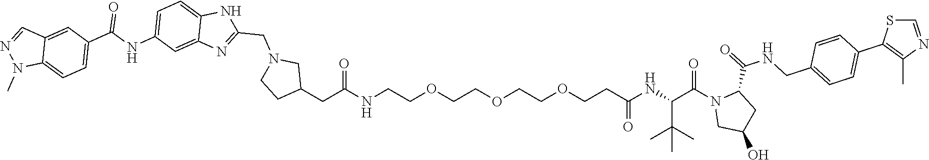 [SMILES: Cc1ncsc1-c1ccc(CNC(=O)[C@@H]2C[C@@H](O)CN2C(=O)[C@@H](NC(=O)CCOCCOCCOCCNC(=O)CC2CCN(Cc3nc4cc(NC(=O)c5ccc6c(cnn6C)c5)ccc4[nH]3)C2)C(C)(C)C)cc1] | N-(2-((3-((S)-17-((2S,4R)-4- hydroxy-2-((4-(4-methylthiazol-5- yl)benzyl)carbamoyl)pyrrolidine-1- carbonyl)-18,18-dimethyl-2,15- dioxo-6,9,12-trioxa-3,16- diazanonadecyl)pyrrolidin-1- yl)methyl)-1H-benzo[d]imidazol-5- yl)-1-methyl-1H-indazole-5- carboxamide |
| 8 | LQ076- 52 | 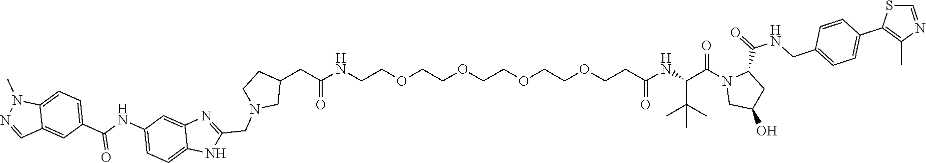 [SMILES: Cc1ncsc1-c1ccc(CNC(=O)[C@@H]2C[C@@H](O)CN2C(=O)[C@@H](NC(=O)CCOCCOCCOCCOCCNC(=O)CC2CCN(Cc3nc4cc(NC(=O)c5ccc6c(cnn6C)c5)ccc4[nH]3)C2)C(C)(C)C)cc1] | N-(2-((3-((S)-20-((2S,4R)-4- hydroxy-2-((4-(4-methylthiazol-5- yl)benzyl)carbamoyl)pyrrolidine-1- carbonyl)-21,21-dimethyl-2,18- dioxo-6,9,12,15-tetraoxa-3,19- diazadocosyl)pyrrolidin-1- yl)methyl)-1H-benzo[d]imidazol-5- yl)-1-methyl-1H-indazole-5- carboxamide |
| 9 | LQ076- 53 | 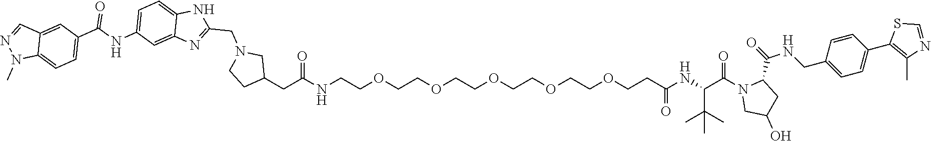 [SMILES: Cc1ncsc1-c1ccc(CNC(=O)[C@@H]2CC(O)CN2C(=O)[C@@H](NC(=O)CCOCCOCCOCCOCCOCCNC(=O)CC2CCN(Cc3nc4cc(NC(=O)c5ccc6c(cnn6C)c5)ccc4[nH]3)C2)C(C)(C)C)cc1] | N-(2-((3-((S)-23-((2S,4R)-4- hydroxy-2-((4-(4-methylthiazol-5- yl)benzyl)carbamoyl)pyrrolidine-1- carbonyl)-24,24-dimethyl-2,21- dioxo-6,9,12,15,18-pentaoxa-3,22- diazapentacosyl)pyrrolidin-1- yl)methyl)-1H-benzo[d]imidazol-5- yl)-1-methyl-1H-indazole-5- carboxamide |
| 10 | LQ076- 54 | 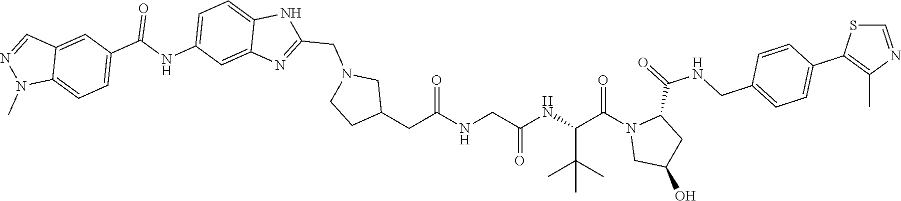 [SMILES: Cc1ncsc1-c1ccc(CNC(=O)[C@@H]2C[C@@H](O)CN2C(=O)[C@@H](NC(=O)CNC(=O)CC2CCN(Cc3nc4cc(NC(=O)c5ccc6c(cnn6C)c5)ccc4[nH]3)C2)C(C)(C)C)cc1] | N-(2-((3-(2-((2-(((S)-1-((2S,4R)-4- hydroxy-2-((4-(4-methylthiazol-5- yl)benzyl)carbamoyl)pyrrolidin-1- yl)-3,3-dimethyl-1-oxobutan-2- yl)amino)-2-oxoethyl)amino)-2- oxoethyl)pyrrolidin-1-yl)methyl)- 1H-benzo[d]imidazol-5-yl)-1- methyl-1H-indazole-5-carboxamide |
| 11 | LQ076- 55 | 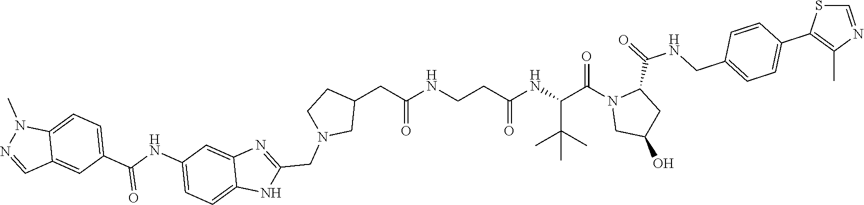 [SMILES: Cc1ncsc1-c1ccc(CNC(=O)[C@@H]2C[C@@H](O)CN2C(=O)[C@@H](NC(=O)CCNC(=O)CC2CCN(Cc3nc4cc(NC(=O)c5ccc6c(cnn6C)c5)ccc4[nH]3)C2)C(C)(C)C)cc1] | N-(2-((3-(2-((3-(((S)-1-((2S,4R)-4- hydroxy-2-((4-(4-methylthiazol-5- yl)benzyl)carbamoyl)pyrrolidin-1- yl)-3,3-dimethyl-1-oxobutan-2- yl)amino)-3-oxopropyl)amino)-2- oxoethyl)pyrrolidin-1-yl)methyl)- 1H-benzo[d]imidazol-5-yl)-1- methyl-1H-indazol-5-carboxamide |
| 12 | LQ076- 56 | 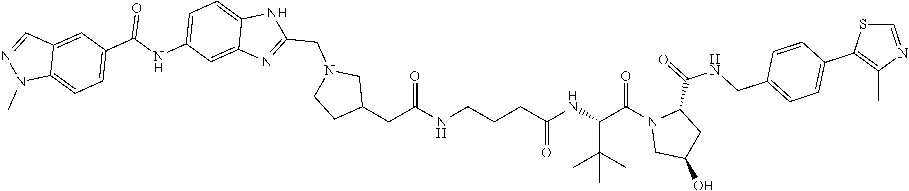 [SMILES: Cc1ncsc1-c1ccc(CNC(=O)[C@@H]2C[C@@H](O)CN2C(=O)[C@@H](NC(=O)CCCNC(=O)CC2CCN(Cc3nc4cc(NC(=O)c5ccc6c(cnn6C)c5)ccc4[nH]3)C2)C(C)(C)C)cc1] | N-(2-((3-(2-((4-(((S)-1-((2S,4R)-4- hydroxy-2-((4-(4-methylthiazol-5- yl)benzyl)carbamoyl)pyrrolidin-1- yl)-3,3-dimethyl-1-oxobutan-2- yl)amino)-4-oxobutyl)amino)-2- oxoethyl)pyrrolidin-1-yl)methyl)- 1H-benzo[d]imidazol-5-yl)-1- methyl-1H-indazole-5-carboxamide |
| 13 | LQ076- 57 | 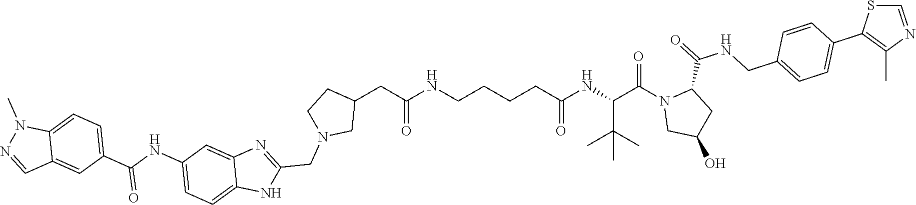 [SMILES: Cc1ncsc1-c1ccc(CNC(=O)[C@@H]2C[C@@H](O)CN2C(=O)[C@@H](NC(=O)CCCCNC(=O)CC2CCN(Cc3nc4cc(NC(=O)c5ccc6c(cnn6C)c5)ccc4[nH]3)C2)C(C)(C)C)cc1] | N-(2-((3-(2-((5-(((S)-1-((2S,4R)-4- hydroxy-2-((4-(4-methylthiazol-5- yl)benzyl)carbamoyl)pyrrolidin-1- yl)-3,3-dimethyl-1-oxobutan-2- yl)amino)-5-oxopentyl)amino)-2- oxoethyl)pyrrolidin-1-yl)methyl)- 1H-benzo[d]imidazol-5-yl)-1- methyl-1H-indazole-5-carboxamide |
| 14 | LQ076- 58 | 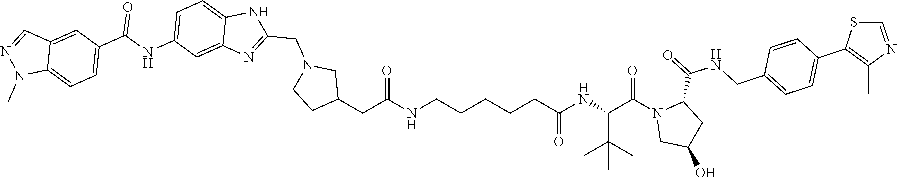 [SMILES: Cc1ncsc1-c1ccc(CNC(=O)[C@@H]2C[C@@H](O)CN2C(=O)[C@@H](NC(=O)CCCCCNC(=O)CC2CCN(Cc3nc4cc(NC(=O)c5ccc6c(cnn6C)c5)ccc4[nH]3)C2)C(C)(C)C)cc1] | N-(2-((3-(2-((6-(((S)-1-((2S,4R)-4- hydroxy-2-((4-(4-methylthiazol-5- yl)benzyl)carbamoyl)pyrrolidin-1- yl)-3,3-dimethyl-1-oxobutan-2- yl)amino)-6-oxohexyl)amino)-2- oxoethyl)pyrrolidin-1-yl)methyl)- 1H-benzo[d]imidazol-5-yl)-1- methyl-1H-indazole-5-carboxamide |
| 15 | LQ076- 59 | 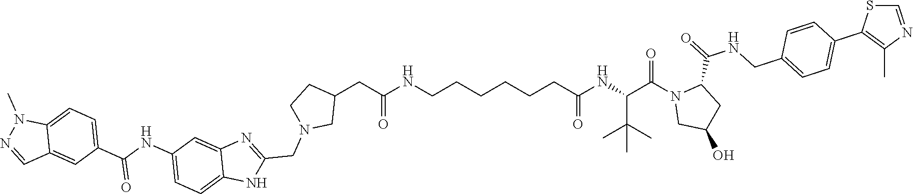 [SMILES: Cc1ncsc1-c1ccc(CNC(=O)[C@@H]2C[C@@H](O)CN2C(=O)[C@@H](NC(=O)CCCCCCNC(=O)CC2CCN(Cc3nc4cc(NC(=O)c5ccc6c(cnn6C)c5)ccc4[nH]3)C2)C(C)(C)C)cc1] | N-(2-((3-(2-((7-(((S)-1-((2S,4R)-4- hydroxy-2-((4-(4-methylthiazol-5- y)benzyl)carbamoyl)pyrrolidin-1- yl)-3,3-dimethyl-1-oxobutan-2- yl)amino)-7-oxoheptyl)amino)-2- oxoethyl)pyrrolidin-1-yl)methyl)- 1H-benzo[d]imidazol-5-yl)-1- methyl-1H-indazole-5-carboxamide |
| 16 | LQ076- 60 | 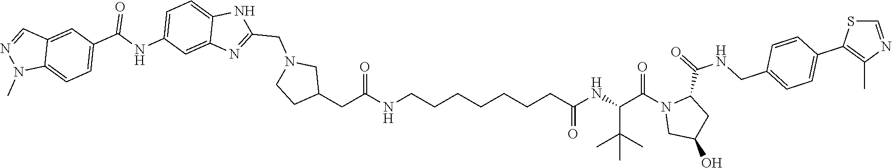 [SMILES: Cc1ncsc1-c1ccc(CNC(=O)[C@@H]2C[C@@H](O)CN2C(=O)[C@@H](NC(=O)CCCCCCCNC(=O)CC2CCN(Cc3nc4cc(NC(=O)c5ccc6c(cnn6C)c5)ccc4[nH]3)C2)C(C)(C)C)cc1] | N-(2-((3-(2-((8-(((S)-1-((2S,4R)-4- hydroxy-2-((4-(4-methylthiazol-5- yl)benzyl)carbamoyl)pyrrolidin-1- yl)-3,3-dimethyl-1-oxobutan-2- yl)amino)-8-oxooctyl)amino)-2- oxoethyl)pyrrolidin-1-yl)methyl)- 1H-benzo[d]imidazol-5-yl)-1- methyl-1H-indazole-5-carboxamide |
| 17 | LQ076- 61 | 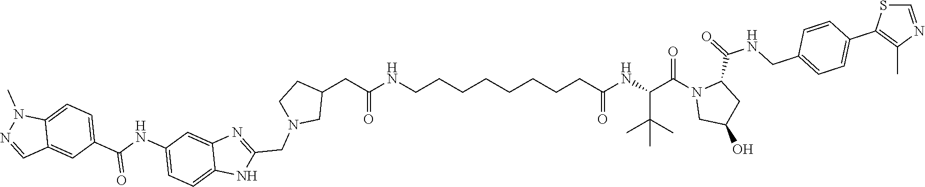 [SMILES: Cc1ncsc1-c1ccc(CNC(=O)[C@@H]2C[C@@H](O)CN2C(=O)[C@@H](NC(=O)CCCCCCCCNC(=O)CC2CCN(Cc3nc4cc(NC(=O)c5ccc6c(cnn6C)c5)ccc4[nH]3)C2)C(C)(C)C)cc1] | N-(2-((3-(2-((9-(((S)-1-((2S,4R)-4- hydroxy-2-((4-(4-methylthiazol-5- yl)benzyl)carbamoyl)pyrrolidin-1- yl)-3,3-dimethyl-1-oxobutan-2- yl)amino)-9-oxononyl)amino)-2- oxoethyl)pyrrolidin-1-yl)methyl)- 1H-benzo[d]imidazol-5-yl)-1- methyl-1H-indazole-5-carboxamide |
| 18 | LQ076- 62 | 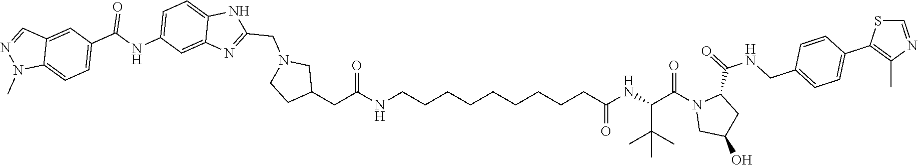 [SMILES: Cc1ncsc1-c1ccc(CNC(=O)[C@@H]2C[C@@H](O)CN2C(=O)[C@@H](NC(=O)CCCCCCCCCNC(=O)CC2CCN(Cc3nc4cc(NC(=O)c5ccc6c(cnn6C)c5)ccc4[nH]3)C2)C(C)(C)C)cc1] | N-(2-((3-(2-((10-(((S)-1-((2S,4R)-4- hydroxy-2-((4-(4-methylthiazol-5- yl)benzyl)carbamoyl)pyrrolidin-1- yl)-3,3-dimethyl-1-oxobutan-2- yl)amino)-10-oxodecyl)amino)-2- oxoethyl)pyrrolidin-1-yl)methyl)- 1H-benzo[d]imidazol-5-yl)-1- methyl-1H-indazole-5-carboxamide |
| 19 | LQ076- 63 | 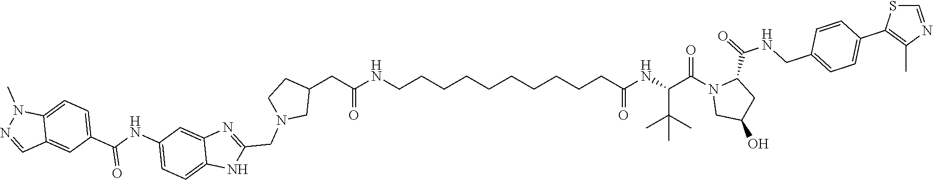 [SMILES: Cc1ncsc1-c1ccc(CNC(=O)[C@@H]2C[C@@H](O)CN2C(=O)[C@@H](NC(=O)CCCCCCCCCCNC(=O)CC2CCN(Cc3nc4cc(NC(=O)c5ccc6c(cnn6C)c5)ccc4[nH]3)C2)C(C)(C)C)cc1] | N-(2-((3-(2-((11-(((S)-1-((2S,4R)-4- hydroxy-2-((4-(4-methylthiazol-5- yl)benzyl)carbamoyl)pyrrolidin-1- yl)-3,3-dimethyl-1-oxobutan-2-yl) amino)-11-oxoundecyl)amino)-2- oxoethyl)pyrrolidin-1-yl)methyl)- 1H-benzo[d]imidazol-5-yl)-1- methyl-1H-indazole-5-carboxamide |
| 20 | LQ076- 64 | 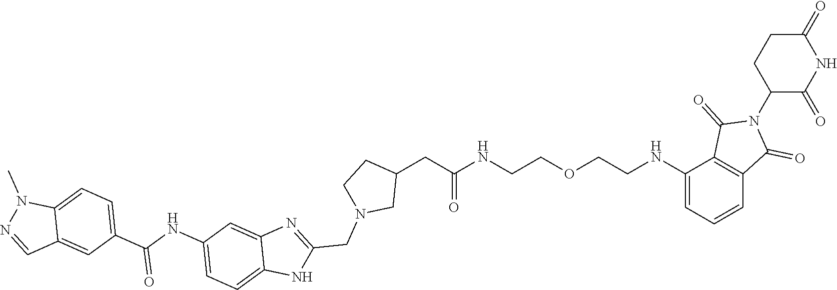 [SMILES: Cn1ncc2cc(C(=O)Nc3ccc4[nH]c(CN5CCC(CC(=O)NCCOCCNc6cccc7c6C(=O)N(C6CCC(=O)NC6=O)C7=O)C5)nc4c3)ccc21] | N-(2-((3-(2-((2-(2-((2-(2,6- dioxopiperidin-3-yl)-1,3- dioxoisoindolin-4- yl)amino)ethoxy)ethyl)amino)-2- oxoethyl)pyrrolidin-1-yl)methyl)- 1H-benzo[d]imidazol-5-yl)-1- methyl-1H-indazole-5-carboxamide |
| 21 | LQ076- 65 | 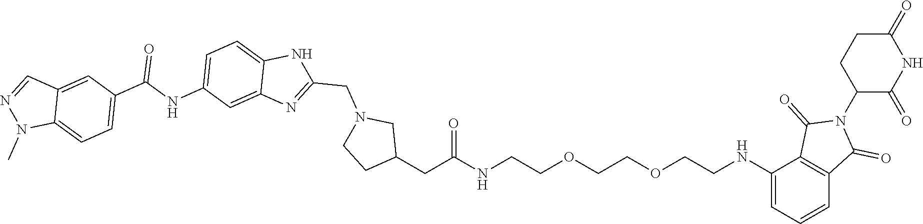 [SMILES: Cn1ncc2cc(C(=O)Nc3ccc4[nH]c(CN5CCC(CC(=O)NCCOCCOCCNc6cccc7c6C(=O)N(C6CCC(=O)NC6=O)C7=O)C5)nc4c3)ccc21] | N-(2-((3-(2-((2-(2-(2-((2-(2,6- dioxopiperidin-3-yl)-1,3- dioxoisoindolin-4-yl)ami- no)ethoxy)ethoxy)ethyl)amino)- 2-oxoethyl)pyrrolidin-1- yl)methyl)-1H-benzo[d]imidazol-5- yl)-1-methyl-1H-indazole-5- carboxamide |
| 22 | LQ076- 66 | 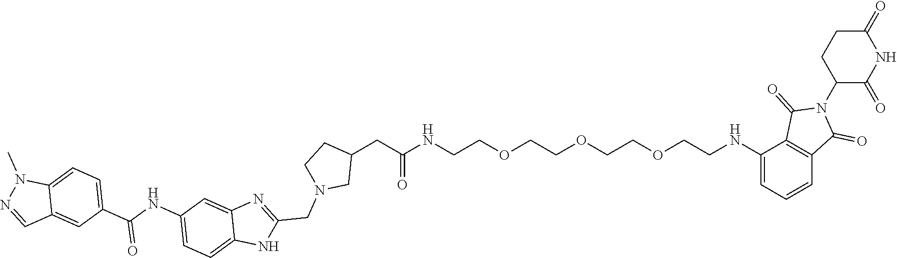 [SMILES: Cn1ncc2cc(C(=O)Nc3ccc4[nH]c(CN5CCC(CC(=O)NCCOCCOCCOCCNc6cccc7c6C(=O)N(C6CCC(=O)NC6=O)C7=O)C5)nc4c3)ccc21] | N-(2-((3-(14-((2-(2,6- dioxopiperidin- 3-yl)-1,3-dioxoisoindolin-4- yl)amino)-2-oxo-6,9,12-trioxa-3- azatetradecyl)pyrrolidin-1- yl)methyl)-1H-benzo[d]imidazol-5- yl)-1-methyl-1H-indazole-5- carboxamide |
| 23 | LQ076- 67 | 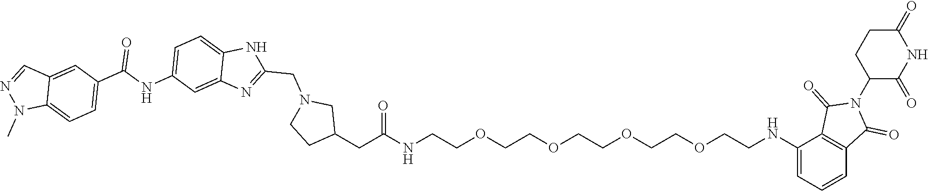 [SMILES: Cn1ncc2cc(C(=O)Nc3ccc4[nH]c(CN5CCC(CC(=O)NCCOCCOCCOCCOCCNc6cccc7c6C(=O)N(C6CCC(=O)NC6=O)C7=O)C5)nc4c3)ccc21] | N-(2-((3-(17-((2-(2,6- dioxopiperidin- 3-yl)-1,3-dioxoisoindolin-4- yl)amino)-2-oxa-6,9,12,15-tetraoxa- 3-azaheptadecyl)pyrrolidin-1- yl)methyl)-1H-benzo[d]imidazol-5- yl)-1-methyl-1H-indazole-5- carboxamide |
| 24 | LQ076- 68 | 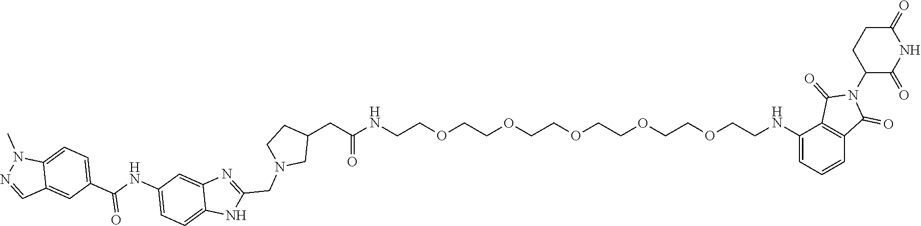 [SMILES: Cn1ncc2cc(C(=O)Nc3ccc4[nH]c(CN5CCC(CC(=O)NCCOCCOCCOCCOCCOCCNc6cccc7c6C(=O)N(C6CCC(=O)NC6=O)C7=O)C5)nc4c3)ccc21] | N-(2-((3-(20-((2-(2,6- dioxopiperidin- 3-yl)-1,3-dioxoisoindolin-4- yl)amino)-2-oxo-6,9,12,15,18- pentaoxa-3-azaicosyl)pyrrolidin-1- yl)methyl)-1H-benzo[d]imidazol-5- yl)-1-methyl-1H-indazole-5- carboxamide |
| 25 | LQ076- 69 | 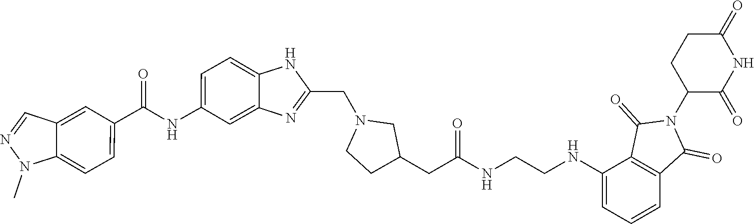 [SMILES: Cn1ncc2cc(C(=O)Nc3ccc4[nH]c(CN5CCC(CC(=O)NCCNc6cccc7c6C(=O)N(C6CCC(=O)NC6=O)C7=O)C5)nc4c3)ccc21] | N-(2-((3-(2-((2-((2-(2,6- dioxopiperidin-3-yl)-1,3- dioxoisoindolin-4- yl)amino)ethyl)amino)-2- oxoethyl)pyrrolidin-1-yl)methyl)- 1H-benzo[d]imidazol-5-yl)-1- methyl-1H-indazole-5-carboxamide |
| 26 | LQ076- 70 | 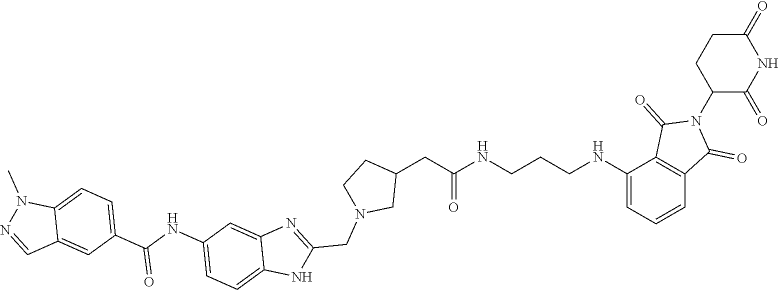 [SMILES: Cn1ncc2cc(C(=O)Nc3ccc4[nH]c(CN5CCC(CC(=O)NCCCNc6cccc7c6C(=O)N(C6CCC(=O)NC6=O)C7=O)C5)nc4c3)ccc21] | N-(2-((3-(2-((3-((2-(2,6- dioxopiperidin-3-yl)-1,3- dioxoisoindolin-4- yl)amino)propyl)amino)-2- oxoethyl)pyrrolidin-1-yl)methyl)- 1H-benzo[d]imidazol-5-yl)-1- methyl-1H-indazole-5-carboxamide |
| 27 | LQ076- 71 | 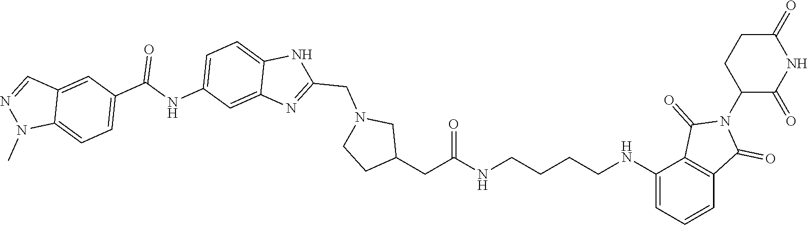 [SMILES: Cn1ncc2cc(C(=O)Nc3ccc4[nH]c(CN5CCC(CC(=O)NCCCCNc6cccc7c6C(=O)N(C6CCC(=O)NC6=O)C7=O)C5)nc4c3)ccc21] | N-(2-((3-(2-((4-((2-(2,6- dioxopiperidin-3-yl)-1,3- dioxoisoindolin-4- yl)amino)butyl)amino)-2- oxoethyl)pyrrolidin-1-yl)methyl)- 1H-benzo[d]imidazol-5-yl)-1- methyl-1H-indazole-5-carboxamide |
| 28 | LQ076- 72 | 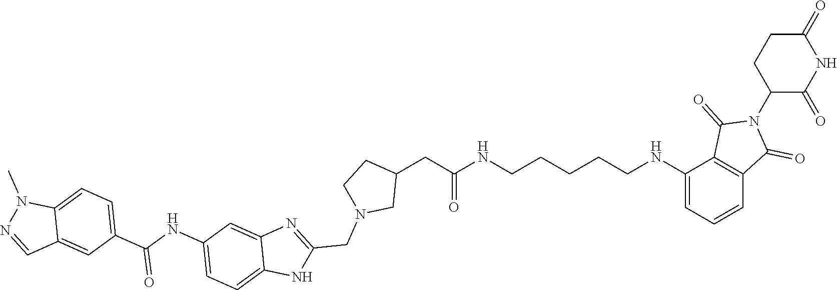 [SMILES: Cn1ncc2cc(C(=O)Nc3ccc4[nH]c(CN5CCC(CC(=O)NCCCCCNc6cccc7c6C(=O)N(C6CCC(=O)NC6=O)C7=O)C5)nc4c3)ccc21] | N-(2-((3-(2-((5-((2-(2,6- dioxopiperidin-3-yl)-1,3- dioxoisoindolin-4- yl)amino)pentyl)amino)-2- oxoethyl)pyrrolidin-1-yl)methyl)- 1H-benzo[d]imidazol-5-yl)-1- methyl-1H-indazole-5-carboxamide |
| 29 | LQ076- 73 | 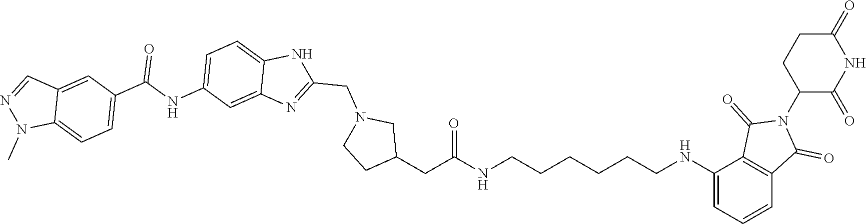 [SMILES: Cn1ncc2cc(C(=O)Nc3ccc4[nH]c(CN5CCC(CC(=O)NCCCCCCNc6cccc7c6C(=O)N(C6CCC(=O)NC6=O)C7=O)C5)nc4c3)ccc21] | N-(2-((3-(2-((6-((2-(2,6- dioxopiperidin-3-yl)-1,3- dioxoisoindolin-4- yl)amino)hexyl)amino)-2- oxoethyl)pyrrolidin-1-yl)methyl)- 1H-benzo[d]imidazol-5-yl)-1- methyl-1H-indazole-5-carboxamide |
| 30 | LQ076- 74 | 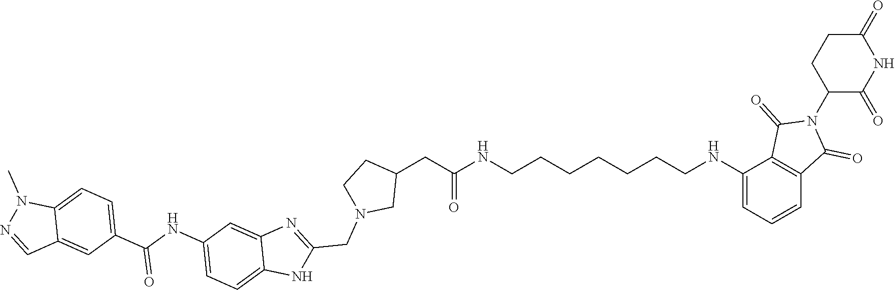 [SMILES: Cn1ncc2cc(C(=O)Nc3ccc4[nH]c(CN5CCC(CC(=O)NCCCCCCCNc6cccc7c6C(=O)N(C6CCC(=O)NC6=O)C7=O)C5)nc4c3)ccc21] | N-(2-((3-(2-((7-((2-(2,6- dioxopiperidin-3-yl)-1,3- dioxoisoindolin-4- yl)amino)heptyl)amino)-2- oxoethyl)pyrrolidin-1-yl)methyl)- 1H-benzo[d]imidazol-5-yl)-1- methyl-1H-indazole-5-carboxamide |
| 31 | LQ076- 75 | 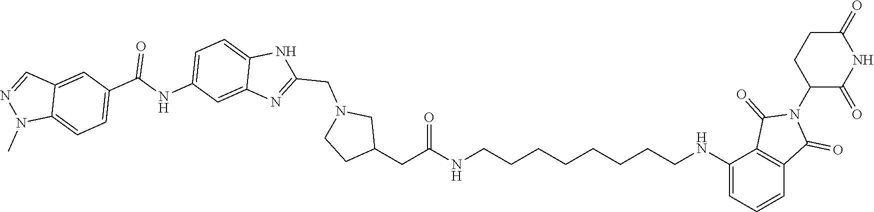 [SMILES: Cn1ncc2cc(C(=O)Nc3ccc4[nH]c(CN5CCC(CC(=O)NCCCCCCCCNc6cccc7c6C(=O)N(C6CCC(=O)NC6=O)C7=O)C5)nc4c3)ccc21] | N-(2-((3-(2-((8-((2-(2,6- dioxopiperidin-3-yl)-1,3- dioxoisoindolin-4- yl)amino)octyl)amino)-2- oxoethyl)pyrrolidin-1-yl)methyl)- 1H-benzo[d]imidazol-5-yl)-1- methyl-1H-indazole-5-carboxamide |
| 33 | LQ076- 76 | 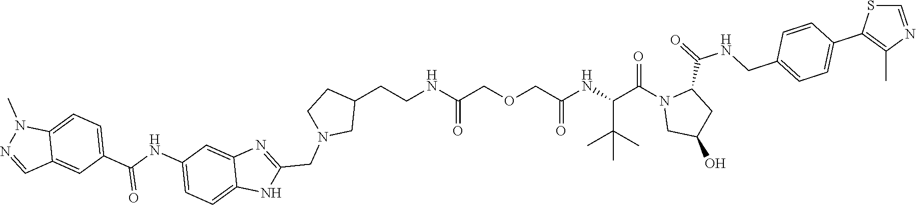 [SMILES: Cc1ncsc1-c1ccc(CNC(=O)[C@@H]2C[C@@H](O)CN2C(=O)[C@@H](NC(=O)COCC(=O)NCCC2CCN(Cc3nc4cc(NC(=O)c5ccc6c(cnn6C)c5)ccc4[nH]3)C2)C(C)(C)C)cc1] | N-(2-((3-(2-(2-(2-(((S)-1-((2S,4R)- 4-hydroxy-2-((4-(4-methylthiazol- 5-yl)benzyl)carbamoyl)pyrroli- din-1-yl)-3,3-dimethyl-1- oxobutan-2-yl)amino)-2-oxo- ethoxy)acetamido)ethyl)pyrrolidin- 1-yl)methyl)-1H- benzo[d]imidazol-5-yl)-1-methyl- 1H-indazole-5-carboxamide |
| 34 | LQ076- 77 | 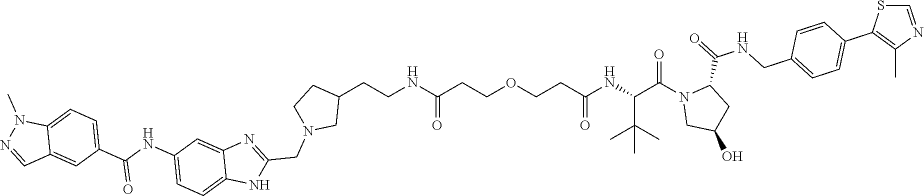 [SMILES: Cc1ncsc1-c1ccc(CNC(=O)[C@@H]2C[C@@H](O)CN2C(=O)[C@@H](NC(=O)CCOCCC(=O)NCCC2CCN(Cc3nc4cc(NC(=O)c5ccc6c(cnn6C)c5)ccc4[nH]3)C2)C(C)(C)C)cc1] | N-(2-((3-(2-(3-(3-(((S)-1-((2S,4R)- 4-hydroxy-2-((4-(4-methylthiazol- 5-yl)benzyl)carbamoyl)pyrroli- din-1-yl)-3,3-dimethyl-1- oxobutan-2-yl)amino)-3- oxopropoxy)propanamido)ethyl) pyrrolidin-1-yl)methyl)-1H- benzo[d]imidazol-5-yl)-1-methyl- 1H-indazole-5-carboxamide |
| 35 | LQ076- 78 | 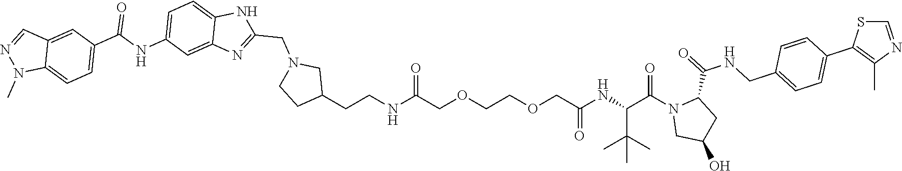 [SMILES: Cc1ncsc1-c1ccc(CNC(=O)[C@@H]2C[C@@H](O)CN2C(=O)[C@@H](NC(=O)COCCOCC(=O)NCCC2CCN(Cc3nc4cc(NC(=O)c5ccc6c(cnn6C)c5)ccc4[nH]3)C2)C(C)(C)C)cc1] | N-(2-((3-((S)-13-((2S,4R)-4-hy- droxy-2-((4-(4-methylthiazol-5- yl)benzyl)carbamoyl)pyrrolidine-1- carbonyl)-14,14-dimethyl-4,11- dioxo-6,9-diox-3,12- diazapentadecyl)pyrrolidin-1- yl)methyl)-1H-benzo[d]imidazol-5- yl)-1-methyl-1H-indazole-5- carboxamide |
| 36 | LQ076- 79 | 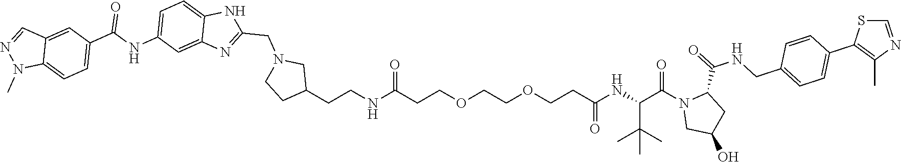 [SMILES: Cc1ncsc1-c1ccc(CNC(=O)[C@@H]2C[C@@H](O)CN2C(=O)[C@@H](NC(=O)CCOCCOCCC(=O)NCCC2CCN(Cc3nc4cc(NC(=O)c5ccc6c(cnn6C)c5)ccc4[nH]3)C2)C(C)(C)C)cc1] | N-(2-((3-((S)-15-((2S,4R)-4-hy- droxy-2-((4-(4-methylthiazol-5- yl)benzyl)carbamoyl)pyrrolidine-1- carbonyl)-16,16-dimethyl-4,13- dioxo-7,10-dioxa-3,14- diazaheptadecyl)pyrrolidin-1- yl)methyl)-1H-benzo[d]imidazol-5- yl)-1-methyl-1H-indazole-5- carboxamide |
| 37 | LQ076- 80 | 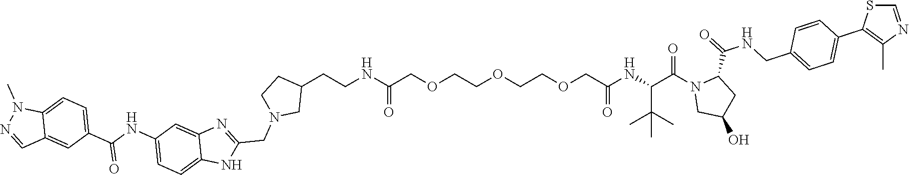 [SMILES: Cc1ncsc1-c1ccc(CNC(=O)[C@@H]2C[C@@H](O)CN2C(=O)[C@@H](NC(=O)COCCOCCOCC(=O)NCCC2CCN(Cc3nc4cc(NC(=O)c5ccc6c(cnn6C)c5)ccc4[nH]3)C2)C(C)(C)C)cc1] | N-(2-((3-((S)-16-((2S,4R)-4-hy- droxy-2-((4-(4-methylthiazol-5- yl)benzyl)carbamoyl)pyrrolidine-1- carbonyl)-17,17-dimethyl-4,14- dioxo-6,9,12-trioxa-3,15- diazaoctadecyl)pyrrolidin-1- yl)methyl)-1H-benzo[d]imidazol-5- yl)-1-methyl-1H-indazole-5- carboxamide |
| 38 | LQ076- 81 | 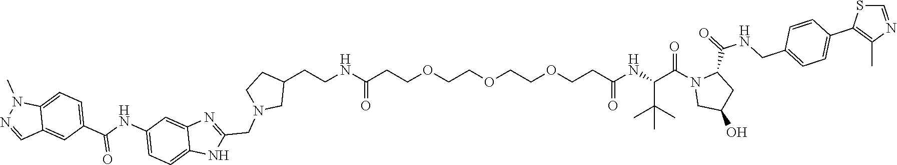 [SMILES: Cc1ncsc1-c1ccc(CNC(=O)[C@@H]2C[C@@H](O)CN2C(=O)[C@@H](NC(=O)CCOCCOCCOCCC(=O)NCCC2CCN(Cc3nc4cc(NC(=O)c5ccc6c(cnn6C)c5)ccc4[nH]3)C2)C(C)(C)C)cc1] | N-(2-((3-((S)-18-((2S,4R)-4-hy- droxy-2-((4-(4-methylthiazol-5- yl)benzyl)carbamoyl)pyrrolidine-1- carbonyl)-19,19-dimethyl-4,16- dioxo-7,10,13-trioxa-3,17- diazaicosyl)pyrrolidin-1-yl)methyl)- 1H-benzo[d]imidazol-5-yl)-1- methyl-1H-indazole-5-carboxamide |
| 39 | LQ076- 82 | 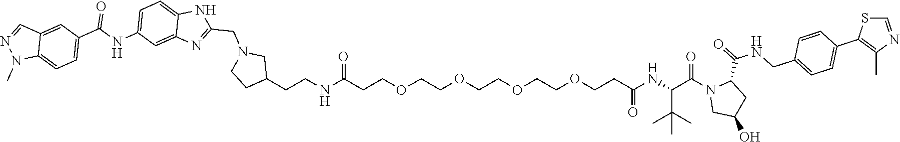 [SMILES: Cc1ncsc1-c1ccc(CNC(=O)[C@@H]2C[C@@H](O)CN2C(=O)[C@@H](NC(=O)CCOCCOCCOCCOCCC(=O)NCCC2CCN(Cc3nc4cc(NC(=O)c5ccc6c(cnn6C)c5)ccc4[nH]3)C2)C(C)(C)C)cc1] | N¹-((S)-1-((2S,4R)-4-hydroxy-2- ((4-(4-methylthiazol-5- yl)benzyl)carbamoyl)pyrrolidin-1- yl)-3,3-dimethyl-1-oxobutan-2-yl)- N¹⁶-(2-(1-((5-(1-methyl-1H- indazole-5-carboxamido)-1H- benzo[d]imidazol-2- yl)methyl)pyrrolidin-3-yl)ethyl)- 4,7,10,13- |
| | | | tetraoxahexadecanediamide |
| 40 | LQ076- 83 | 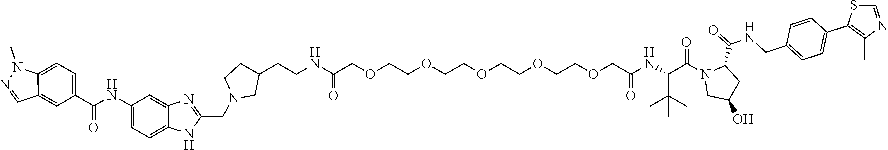 [SMILES: Cc1ncsc1-c1ccc(CNC(=O)[C@@H]2C[C@@H](O)CN2C(=O)[C@@H](NC(=O)COCCOCCOCCOCCOCC(=O)NCCC2CCN(Cc3nc4cc(NC(=O)c5ccc6c(cnn6C)c5)ccc4[nH]3)C2)C(C)(C)C)cc1] | N¹-((S)-1-((2S,4R)-4-hydroxy-2- ((4-(4-methylthiazol-4- yl)benzyl)carbamoyl)pyrrolidin-1- yl)-3,3-dimethyl-1-oxobutan-2-yl)- N¹⁷-(2-(1-((5-(1-methyl-1H- indazole-5-carboxamido)-1H- benzo[d]imidazol-2- yl)methyl)pyrrolidin-3-yl)ethyl)- 3,6,9,12,15- pentaoxaheptadecanediamide |
| 41 | LQ076- 84 | 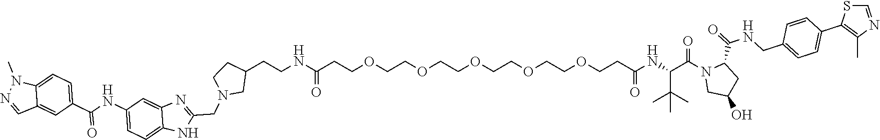 [SMILES: Cc1ncsc1-c1ccc(CNC(=O)[C@@H]2C[C@@H](O)CN2C(=O)[C@@H](NC(=O)CCOCCOCCOCCOCCOCCC(=O)NCCC2CCN(Cc3nc4cc(NC(=O)c5ccc6c(cnn6C)c5)ccc4[nH]3)C2)C(C)(C)C)cc1] | N¹-((S)-1-((2S,4R)-4-hydroxy-2- ((4-(4-methylthiazol-5- yl)benzyl)carbamoyl)pyrrolidin-1- yl)-3,3-dimethyl-1-oxobutan-2-yl)- N¹⁹-(2-(1-((5-(1-methyl-1H- indazole-5-carboxamido)-1H- benzo[d]imidazol-2- yl)methyl)pyrrolidin-3-yl)ethyl)- 4,7,10,13,16- |
| | | | pentaoxanonadecanediamide |
| 42 | LQ076- 85 | 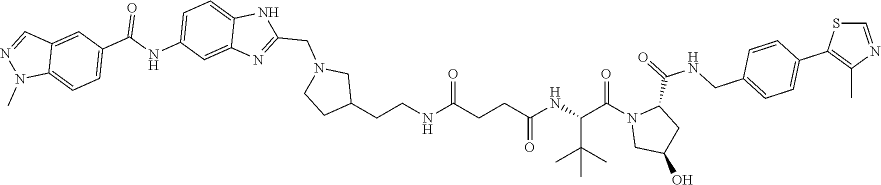 [SMILES: Cc1ncsc1-c1ccc(CNC(=O)[C@@H]2C[C@@H](O)CN2C(=O)[C@@H](NC(=O)CCC(=O)NCCC2CCN(Cc3nc4cc(NC(=O)c5ccc6c(cnn6C)c5)ccc4[nH]3)C2)C(C)(C)C)cc1] | N¹-((S)-1-((2S,4R)-4-hydroxy-2- ((4-(4-methylthiazol-5- yl)benzyl)carbamoyl)pyrrolidin-1- yl)-3,3-dimethyl-1-oxobutan-2-yl)- N⁴-(2-(1-((5-(1-methyl-1H- indazole-5-carboxamido)-1H- benzo[d]imidazol-2- yl)methyl)pyrrolidin-3- yl)ethyl)succinamide |
| 43 | LQ076- 86 | 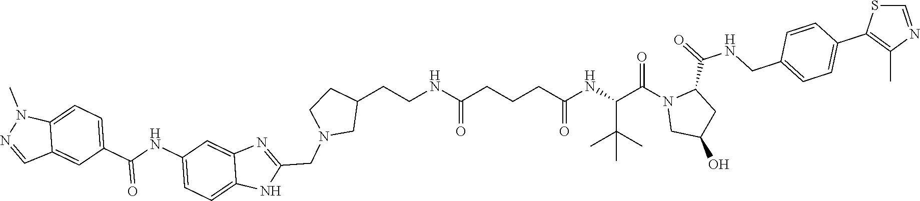 [SMILES: Cc1ncsc1-c1ccc(CNC(=O)[C@@H]2C[C@@H](O)CN2C(=O)[C@@H](NC(=O)CCCC(=O)NCCC2CCN(Cc3nc4cc(NC(=O)c5ccc6c(cnn6C)c5)ccc4[nH]3)C2)C(C)(C)C)cc1] | N¹-((S)-1-((2S,4R)-4-hydroxy-2- ((4-(4-methylthiazol-5- yl)benzyl)carbamoyl)pyrrolidin-1- yl)-3,3-dimethyl-1-oxobutan-2-yl)- N⁵-(2-(1-((5-(1-methyl-1H- indazole-5-carboxamido)-1H- benzo[d]imidazol-2- yl)methyl)pyrrolidin-3- yl)ethyl)glutaramide |
| 44 | LQ076- 87 | 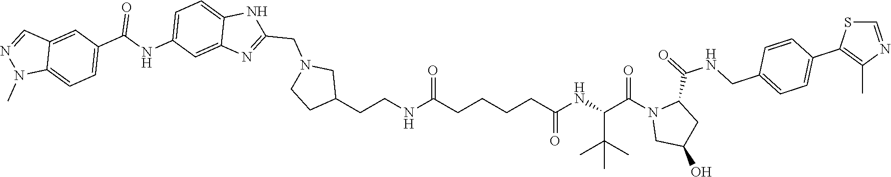 [SMILES: Cc1ncsc1-c1ccc(CNC(=O)[C@@H]2C[C@@H](O)CN2C(=O)[C@@H](NC(=O)CCCCC(=O)NCCC2CCN(Cc3nc4cc(NC(=O)c5ccc6c(cnn6C)c5)ccc4[nH]3)C2)C(C)(C)C)cc1] | N¹-((S)-1-((2S,4R)-4-hydroxy-2- ((4-(4-methylthiazol-5- yl)benzyl)carbamoyl)pyrrolidin-1- yl)-3,3-dimethyl-1-oxobutan-2-yl)- N⁶-(2-(1-((5-(1-methyl-1H- indazole-5-carboxamido)-1H- benzo[d]imidazol-2- yl)methyl)pyrrolidin-3- yl)ethyl)adipamide |
| 45 | LQ076- 88 | 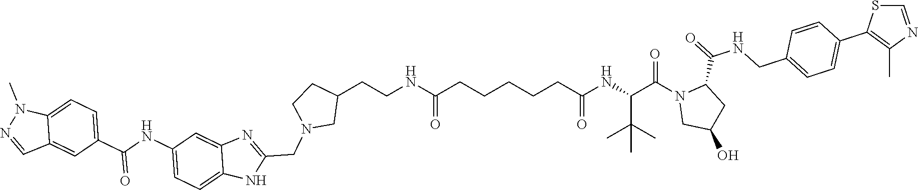 [SMILES: Cc1ncsc1-c1ccc(CNC(=O)[C@@H]2C[C@@H](O)CN2C(=O)[C@@H](NC(=O)CCCCCC(=O)NCCC2CCN(Cc3nc4cc(NC(=O)c5ccc6c(cnn6C)c5)ccc4[nH]3)C2)C(C)(C)C)cc1] | N¹-((S)-1-((2S,4R)-4-hydroxy-2- ((4-(4-methylthiazol-5- yl)benzyl)carbamoyl)pyrrolidin-1- yl)-3,3-dimethyl-1-oxobutan-2-yl)- N⁷-(2-(1-((5-(1-methyl-1H- indazole-5-carboxamido)-1H- benzo[d]imidazol-2- yl)methyl)pyrrolidin-3- yl)ethyl)haptanediamide |
| 46 | LQ076- 89 | 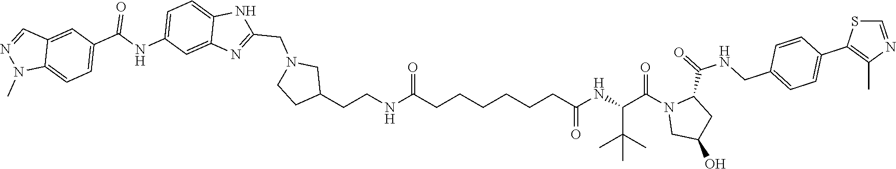 [SMILES: Cc1ncsc1-c1ccc(CNC(=O)[C@@H]2C[C@@H](O)CN2C(=O)[C@@H](NC(=O)CCCCCCC(=O)NCCC2CCN(Cc3nc4cc(NC(=O)c5ccc6c(cnn6C)c5)ccc4[nH]3)C2)C(C)(C)C)cc1] | N¹-((S)-1-((2S,4R)-4-hydroxy-2- ((4-(4-methylthiazol-5- yl)benzyl)carbamoyl)pyrrolidin-1- yl)-3,3-dimethyl-1-oxobutan-2-yl)- N⁸-(2-(1-((5-(1-methyl-1H- indazole-5-carboxamido)-1H- benzo[d]imidazol-2- yl)methyl)pyrrolidin-3- yl)ethyl)octanediamide |
| 47 | LQ076- 90 | 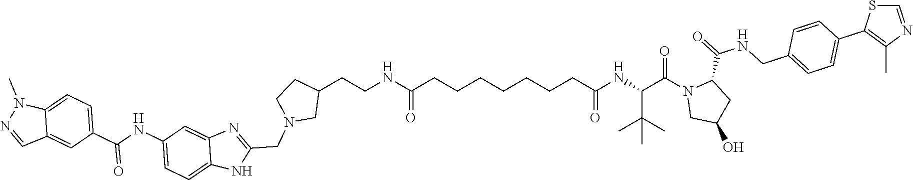 [SMILES: Cc1ncsc1-c1ccc(CNC(=O)[C@@H]2C[C@@H](O)CN2C(=O)[C@@H](NC(=O)CCCCCCCC(=O)NCCC2CCN(Cc3nc4cc(NC(=O)c5ccc6c(cnn6C)c5)ccc4[nH]3)C2)C(C)(C)C)cc1] | N¹-((S)-1-((2S,4R)-4-hydroxy-2- ((4-(4-methylthiazol-5- yl)benzyl)carbamoyl)pyrrolidin-1- yl)-3,3-dimethyl-1-oxobutan-2-yl)- N⁹-(2-(1-((5-(1-methyl-1H- indazole-5-carboxamido)-1H- benzo[d]imidazol-2- yl)methyl)pyrrolidin-3- yl)ethyl)nonanediamide |
| 48 | LQ076- 91 | 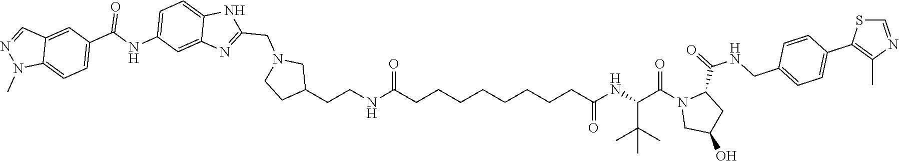 [SMILES: Cc1ncsc1-c1ccc(CNC(=O)[C@@H]2C[C@@H](O)CN2C(=O)[C@@H](NC(=O)CCCCCCCCC(=O)NCCC2CCN(Cc3nc4cc(NC(=O)c5ccc6c(cnn6C)c5)ccc4[nH]3)C2)C(C)(C)C)cc1] | N¹-((S)-1-((2S,4R)-4-hydroxy-2- ((4-(4-methylthiazol-5- yl)benzyl)carbamoyl)pyrrolidin-1- yl)-3,3-dimethyl-1-oxobutan-2-yl)- N¹⁰-(2-(1-((5-(1-methyl-1H- indazole-5-carboxamido)-1H- benzo[d]imidazol-2- yl)methyl)pyrrolidin-3- yl)ethyl)decanediamide |
| 49 | LQ076- 92 | 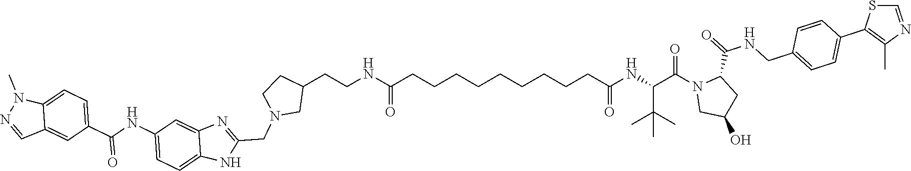 [SMILES: Cc1ncsc1-c1ccc(CNC(=O)[C@@H]2C[C@@H](O)CN2C(=O)[C@@H](NC(=O)CCCCCCCCCC(=O)NCCC2CCN(Cc3nc4cc(NC(=O)c5ccc6c(cnn6C)c5)ccc4[nH]3)C2)C(C)(C)C)cc1] | N¹-((S)-1-((2S,4R)-4-hydroxy-2- ((4-(4-methylthiazol-5- yl)benzyl)carbamoyl)pyrrolidin-1- yl)-3,3-dimethyl-1-oxobutan-2-yl)- N¹¹-(2-(1-((5-(1-methyl-1H- indazole-5-carboxamido)-1H- benzo[d]imidazol-2- yl)methyl)pyrrolidin-3- yl)ethyl)undecanediamide |
| 50 | LQ076- 93 | 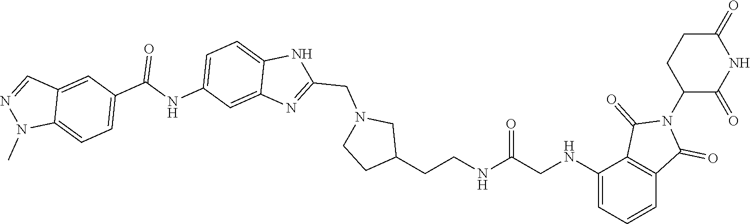 [SMILES: Cn1ncc2cc(C(=O)Nc3ccc4[nH]c(CN5CCC(CCNC(=O)CNc6cccc7c6C(=O)N(C6CCC(=O)NC6=O)C7=O)C5)nc4c3)ccc21] | N-(2-((3-(2-(2-((2-(2,6- dioxopiperidin-3-yl)-1,3- dioxoisoindolin-4-yl) amino)acetamido)ethyl)pyrrolidin- 1-yl)methyl)-1H- benzo[d]imidazol-5-yl)-1-methyl- 1H-indazole-5-carboxamide |
| 51 | LQ076- 94 | 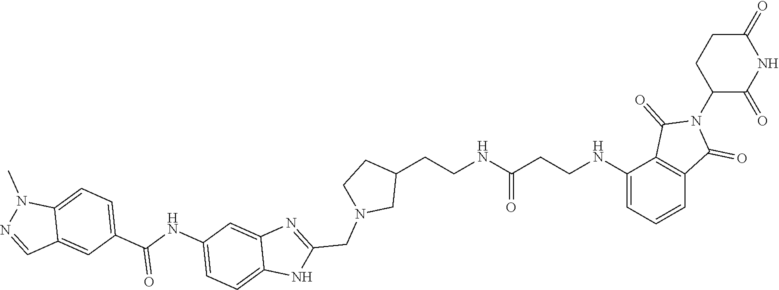 [SMILES: Cn1ncc2cc(C(=O)Nc3ccc4[nH]c(CN5CCC(CCNC(=O)CCNc6cccc7c6C(=O)N(C6CCC(=O)NC6=O)C7=O)C5)nc4c3)ccc21] | N-(2-((3-(2-(3-((2-(2,6- dioxopiperidin-3-yl)-1,3- dioxoisoindolin-4-yl) amino)propanamido)ethyl)pyrroli- din-1-yl)methyl)-1H- benzo[d]imidazol-5-yl)-1-methyl- 1H-indazole-5-carboxamide |
| 52 | LQ076- 95 | 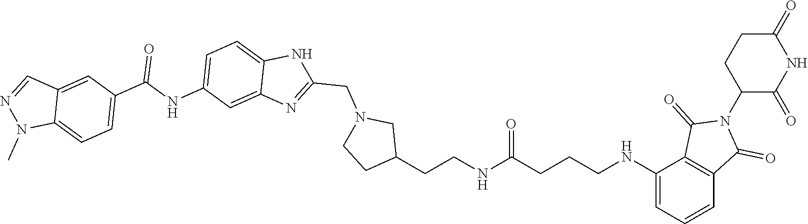 [SMILES: Cn1ncc2cc(C(=O)Nc3ccc4[nH]c(CN5CCC(CCNC(=O)CCCNc6cccc7c6C(=O)N(C6CCC(=O)NC6=O)C7=O)C5)nc4c3)ccc21] | N-(2-((3-(2-(4-((2-(2,6- dioxopiperidin-3-yl)-1,3- dioxoisoindolin-4- yl)amino)butanamido)ethyl)pyrroli- din-1-yl)methyl)-1H- benzo[d]imidazol-5-yl)-1-methyl- 1H-indazole-5-carboxamide |
| 53 | LQ076- 96 | 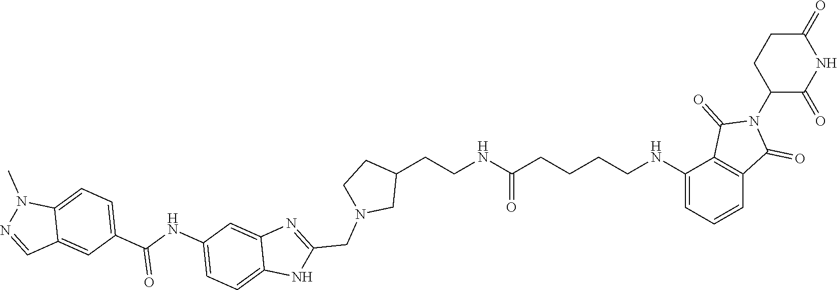 [SMILES: Cn1ncc2cc(C(=O)Nc3ccc4[nH]c(CN5CCC(CCNC(=O)CCCCNc6cccc7c6C(=O)N(C6CCC(=O)NC6=O)C7=O)C5)nc4c3)ccc21] | N-(2-((3-(2-(5-((2-(2,6- dioxopiperidin-3-yl)-1,3- dioxoisoindolin-4-yl) amino)pentanamido)ethyl)pyrroli- din-1-yl)methyl)-1H- benzo[d]imidazol-5-yl)-1-methyl- 1H-indazole-5-carboxamide |
| 54 | LQ076- 97 | 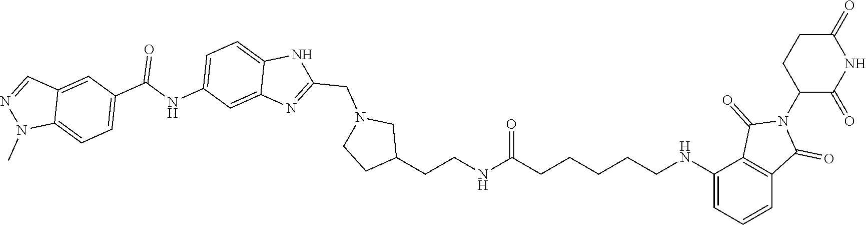 [SMILES: Cn1ncc2cc(C(=O)Nc3ccc4[nH]c(CN5CCC(CCNC(=O)CCCCCNc6cccc7c6C(=O)N(C6CCC(=O)NC6=O)C7=O)C5)nc4c3)ccc21] | N-(2-((3-(2-(6-((2-(2,6- dioxopiperidin-3-yl)-1,3- dioxoisoindolin-4-yl) amino)hexanamido)ethyl)pyrroli- din-1-yl)methyl)-1H- benzo[d]imidazol-5-yl)-1-methyl- 1H-indazole-5-carboxamide |
| 55 | LQ076- 98 | 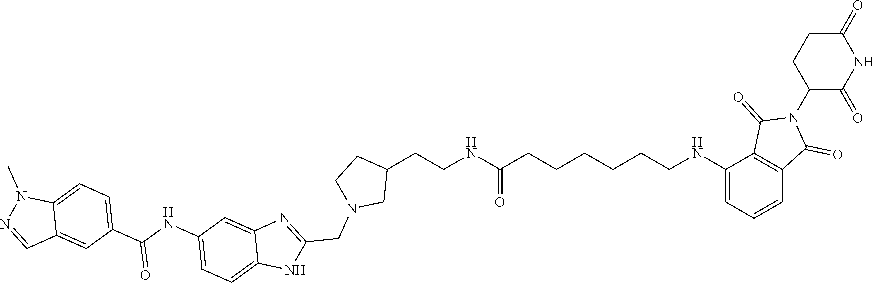 [SMILES: Cn1ncc2cc(C(=O)Nc3ccc4[nH]c(CN5CCC(CCNC(=O)CCCCCCNc6cccc7c6C(=O)N(C6CCC(=O)NC6=O)C7=O)C5)nc4c3)ccc21] | N-(2-((3-(2-(7-((2-(2,6- dioxopiperidin-3-yl)-1,3- dioxoisoindolin-4-yl) amino)heptanamido)ethyl)pyrroli- din-1-yl)methyl)-1H- benzo[d]imidazol-5-yl)-1-methyl- 1H-indazole-5-carboxamide |
| 56 | LQ076- 99 | 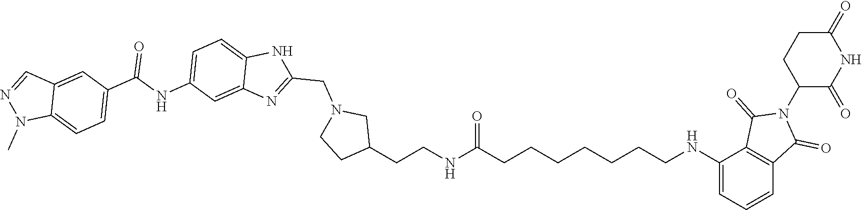 [SMILES: Cn1ncc2cc(C(=O)Nc3ccc4[nH]c(CN5CCC(CCNC(=O)CCCCCCCNc6cccc7c6C(=O)N(C6CCC(=O)NC6=O)C7=O)C5)nc4c3)ccc21] | N-(2-((3-(2-(8-((2-(2,6- dioxopiperidin-3-yl)-1,3- dioxoisoindolin-4-yl)ami- no)octanamido)ethyl)pyrrolidin- 1-yl)methyl)-1H- benzo[d]imidazol-5-yl)-1-methyl- 1H-indazole-5-carboxamide |
| 57 | LQ076- 100 | 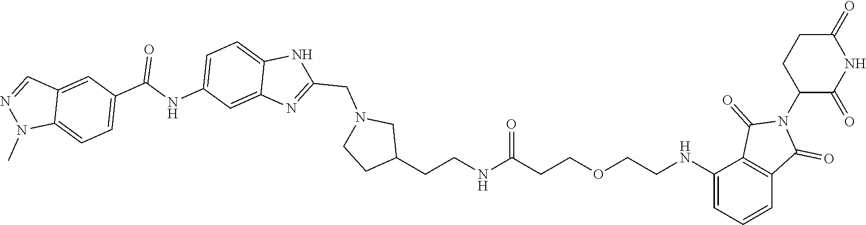 [SMILES: Cn1ncc2cc(C(=O)Nc3ccc4[nH]c(CN5CCC(CCNC(=O)CCOCCNc6cccc7c6C(=O)N(C6CCC(=O)NC6=O)C7=O)C5)nc4c3)ccc21] | N-(2-((3-(2-(3-(2-((2-(2,6- dioxopiperidin-3-yl)-1,3- dioxoisoindolin-4-yl)ami- no)ethoxy)propanamido)ethyl) pyrrolidin-1-yl)methyl)-1H- benzo[d]imidazol-5-yl)-1-methyl- 1H-indazole-5-carboxamide |
| 58 | LQ076- 101 | 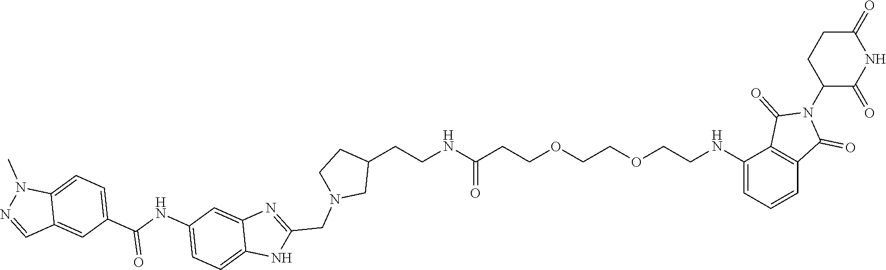 [SMILES: Cn1ncc2cc(C(=O)Nc3ccc4[nH]c(CN5CCC(CCNC(=O)CCOCCOCCNc6cccc7c6C(=O)N(C6CCC(=O)NC6=O)C7=O)C5)nc4c3)ccc21] | N-(2-((3-(2-(3-(2-(2-((2-(2,6- dioxopiperidin-3-yl)-1,3- dioxoisoindolin-4-yl) amino)ethoxy)ethoxy)propanami- do)ethyl)pyrrolidin-1-yl)methyl)- 1H-benzo[d]imidazol-5-yl)-1- methyl-1H-indazole-5-carboxamide |
| 59 | LQ076- 102 | 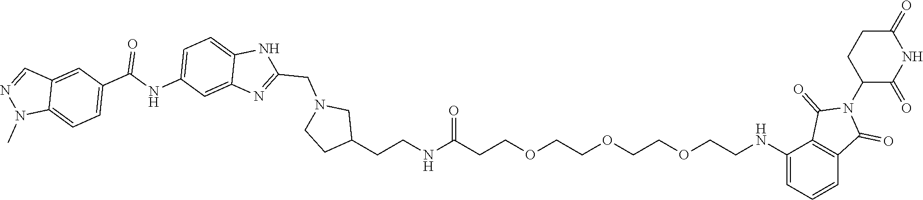 [SMILES: Cn1ncc2cc(C(=O)Nc3ccc4[nH]c(CN5CCC(CCNC(=O)CCOCCOCCOCCNc6cccc7c6C(=O)N(C6CCC(=O)NC6=O)C7=O)C5)nc4c3)ccc21] | N-(2-((3-(1-((2-(2,6-dioxopiperidin- 3-yl)-1,3-dioxoisoindolin-4- yl)amino)-12-oxo-3,6,9-trioxa-13- azapentadecan-15-yl)pyrrolidin-1- yl)methyl)-1H-benzo[d]imidazol-5- yl)-1-methyl-1H-indazole-5- carboxamide |
| 60 | LQ076- 103 | 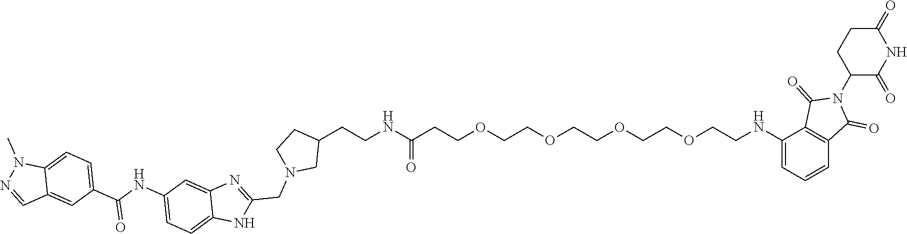 [SMILES: Cn1ncc2cc(C(=O)Nc3ccc4[nH]c(CN5CCC(CCNC(=O)CCOCCOCCOCCOCCNc6cccc7c6C(=O)N(C6CCC(=O)NC6=O)C7=O)C5)nc4c3)ccc21] | N-(2-((3-(1-((2-(2,6-dioxopiperidin- 3-yl)-1,3-dioxoisoindolin-4- yl)amino)-15-oxo-3,6,9,12-tetraoxa- 16-azaoctadecan-18-yl)pyrrolidin-1- yl)methyl)-1H-benzo[d]imidazol-5- yl)-1-methyl-1H-indazole-5- carboxamide |
| 61 | LQ076- 104 | 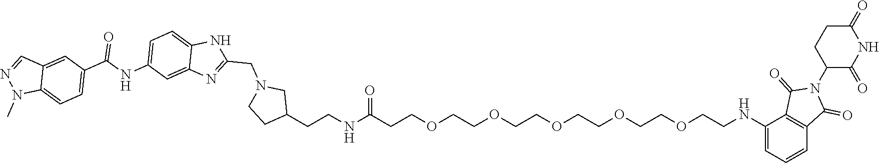 [SMILES: Cn1ncc2cc(C(=O)Nc3ccc4[nH]c(CN5CCC(CCNC(=O)CCOCCOCCOCCOCCOCCNc6cccc7c6C(=O)N(C6CCC(=O)NC6=O)C7=O)C5)nc4c3)ccc21] | N-(2-((3-(1-((2-(2,6-dioxopiperidin- 3-yl)-1,3-dioxoisoindolin-4- yl)amino)-18-oxo-3,6,9,12,15- pentaoxa-19-azahenicosan-21- yl)pyrrolidin-1-yl)methyl)-1H- benzo[d]imidazol-5-yl)-1-methyl- 1H-indazole-5-carboxamide |
| 63 | LQ076- 105 | 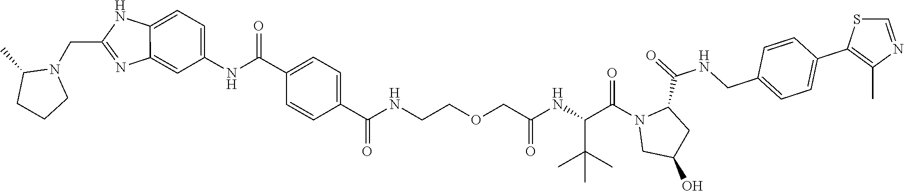 [SMILES: Cc1ncsc1-c1ccc(CNC(=O)[C@@H]2C[C@@H](O)CN2C(=O)[C@@H](NC(=O)COCCNC(=O)c2ccc(C(=O)Nc3ccc4[nH]c(CN5CCC[C@@H]5C)nc4c3)cc2)C(C)(C)C)cc1] | N¹-(2-(2-(((S)-1-((2S,4R)-4- hydroxy-2-((4-(4-methylthiazol-5- yl)benzyl)carbamoyl)pyrrolidin-1- yl)-3,3-dimethyl-1-oxobutan-2- yl)amino)-2-oxoethoxy)ethyl)-N⁴- (2-(((S)-2-methylpyrrolidin-1- yl)methyl)-1H-benzo[d]imidazol-5- yl)terephthalamide |
| 64 | LQ076- 106 | 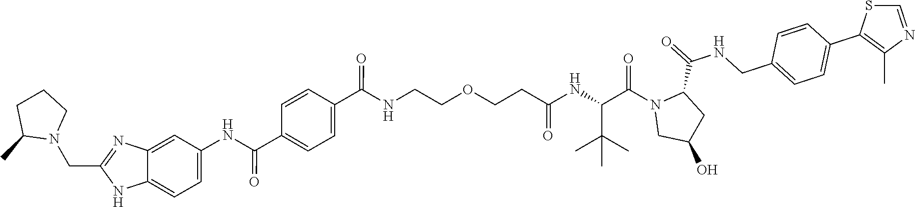 [SMILES: Cc1ncsc1-c1ccc(CNC(=O)[C@@H]2C[C@@H](O)CN2C(=O)[C@@H](NC(=O)CCOCCNC(=O)c2ccc(C(=O)Nc3ccc4[nH]c(CN5CCC[C@@H]5C)nc4c3)cc2)C(C)(C)C)cc1] | N¹-(2-(3-(((S)-1-((2S,4R)-4- hydroxy-2-((4-(4-methylthiazol-5- yl)benzyl)carbamoyl)pyrrolidin-1- yl)-3,3-dimethyl-1-oxobutan-2- yl)amino)-3-oxopropoxy)ethyl)-N⁴- (2-(((S)-2-methylpyrrolidin-1- yl)methyl)-1H-benzo[d]imidazol-5- yl)terephthalamide |
| 65 | LQ076- 107 | 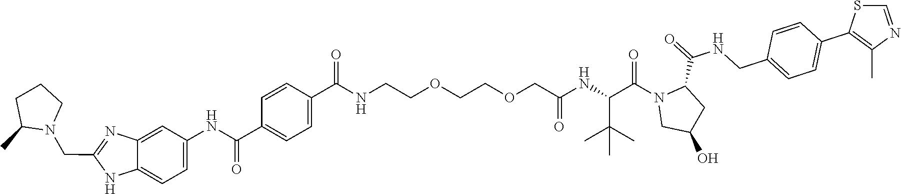 [SMILES: Cc1ncsc1-c1ccc(CNC(=O)[C@@H]2C[C@@H](O)CN2C(=O)[C@@H](NC(=O)COCCOCCNC(=O)c2ccc(C(=O)Nc3ccc4[nH]c(CN5CCC[C@@H]5C)nc4c3)cc2)C(C)(C)C)cc1] | N¹-(2-(2-(2-(((S)-1-((2S,4R)-4- hydroxy-2-((4-(4-methylthiazol-5- yl)benzyl)carbamoyl)pyrrolidin-1- yl)-3,3-dimethyl-1-oxobutan-2- yl)amino)-2-oxo- ethoxy)ethoxy)ethyl)-N⁴-(2-(((S)- 2-methylpyrrolidin-1-yl)methyl)- 1H-benzo[d]imidazol-5- yl)terephthalamide |
| 66 | LQ076- 108 | 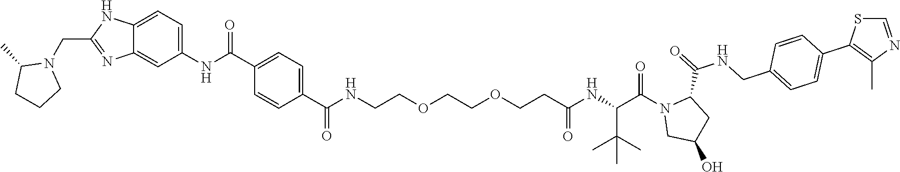 [SMILES: Cc1ncsc1-c1ccc(CNC(=O)[C@@H]2C[C@@H](O)CN2C(=O)[C@@H](NC(=O)CCOCCOCCNC(=O)c2ccc(C(=O)Nc3ccc4[nH]c(CN5CCC[C@@H]5C)nc4c3)cc2)C(C)(C)C)cc1] | N¹-(2-(2-(3-(((S)-1-((2S,4R)-4- hydroxy-2-((4-(4-methylthiazol-5- yl)benzyl)carbamoyl)pyrrolidin-1- yl)-3,3-dimethyl-1-oxobutan-2- yl)amino)-3- oxopropoxy)ethoxy)ethyl)-N⁴-(2- (((S)-2-methylpyrrolidin-1- yl)methyl)-1H-benzo[d]imidazol-5- yl)terephthalamide |
| 67 | LQ076- 109 | 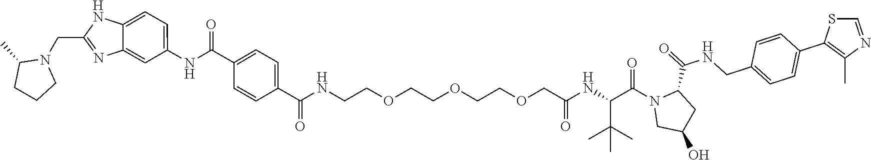 [SMILES: Cc1ncsc1-c1ccc(CNC(=O)[C@@H]2C[C@@H](O)CN2C(=O)[C@@H](NC(=O)COCCOCCOCCNC(=O)c2ccc(C(=O)Nc3ccc4[nH]c(CN5CCC[C@@H]5C)nc4c3)cc2)C(C)(C)C)cc1] | N¹-((S)-13-((2S,4R)-4-hydroxy-2- ((4-(4-methylthiazol-5- yl)benzyl)carbamoyl)pyrrolidine-1- carbonyl)-14,14-dimethyl-11-oxo- 3,6,9-trioxa-12-azapentadecyl)-N⁴- (2-(((S)-2-methylpyrrolidin-1- yl)methyl)-1H-benzo[d]imidazol-5- yl)terephthalamide |
| 68 | LQ076- 110 | 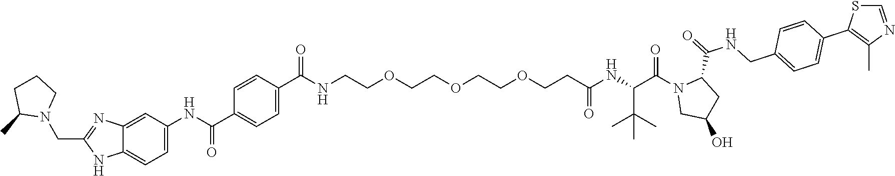 [SMILES: Cc1ncsc1-c1ccc(CNC(=O)[C@@H]2C[C@@H](O)CN2C(=O)[C@@H](NC(=O)CCOCCOCCOCCNC(=O)c2ccc(C(=O)Nc3ccc4[nH]c(CN5CCC[C@@H]5C)nc4c3)cc2)C(C)(C)C)cc1] | N¹-((S)-14-((2S,4R)-4-hydroxy-2- ((4-(4-methylthiazol-5- yl)benzyl)carbamoyl)pyrrolidine-1- carbonyl)-15,15-dimethyl-12-oxo- 3,6,9-trioxa-13-azahexadecyl)-N⁴- (2-(((S)-2-methylpyrrolidin-1- yl)methyl)-1H-benzo[d]imidazol-5- yl)terephthalamide |
| 69 | LQ076- 111 | 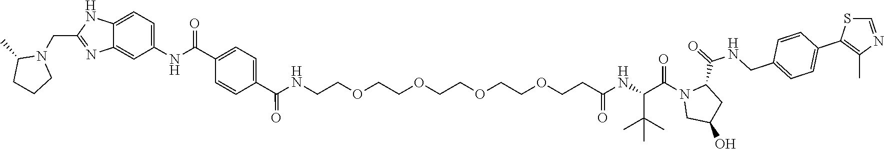 [SMILES: Cc1ncsc1-c1ccc(CNC(=O)[C@@H]2C[C@@H](O)CN2C(=O)[C@@H](NC(=O)CCOCCOCCOCCOCCNC(=O)c2ccc(C(=O)Nc3ccc4[nH]c(CN5CCC[C@@H]5C)nc4c3)cc2)C(C)(C)C)cc1] | N¹-((S)-17-((2S,4R)-4-hydroxy-2- ((4-(4-methylthiazol-5- yl)benzyl)carbamoyl)pyrrolidine-1- carbonyl)-18,18-dimethyl-15-oxo- 3,6,9,12-tetraoxa-16-azanonadecyl)- N⁴-(2-(((S)-2-methylpyrrolidin-1- yl)methyl)-1H-benzo[d]imidazol-5- yl)terephthalamide |
| 70 | LQ076- 112 | 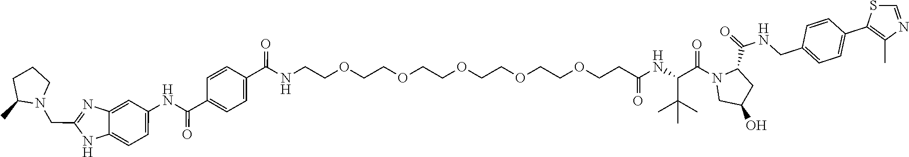 [SMILES: Cc1ncsc1-c1ccc(CNC(=O)[C@@H]2C[C@@H](O)CN2C(=O)[C@@H](NC(=O)CCOCCOCCOCCOCCOCCNC(=O)c2ccc(C(=O)Nc3ccc4[nH]c(CN5CCC[C@@H]5C)nc4c3)cc2)C(C)(C)C)cc1] | N¹-((S)-20-((2S,4R)-4-hydroxy-2- ((4-(4-methylthiazol-5- yl)benzyl)carbamoyl)pyrrolidine-1- carbonyl)-21,21-dimethyl-18-oxo- 3,6,9,12,15-pentaoxa-19- azadocosyl)-N⁴-(2-(((S)-2- methylpyrrolidin-1-yl)methyl)-1H- benzo[d]imidazol-5- yl)terephthalamide |
| 71 | LQ076- 113 | 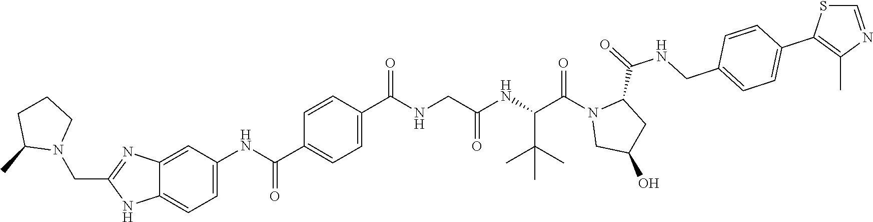 [SMILES: Cc1ncsc1-c1ccc(CNC(=O)[C@@H]2C[C@@H](O)CN2C(=O)[C@@H](NC(=O)CNC(=O)c2ccc(C(=O)Nc3ccc4[nH]c(CN5CCC[C@@H]5C)nc4c3)cc2)C(C)(C)C)cc1] | N¹-(2-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2- yl)amino)-2-oxoethyl)-N⁴-(2-(((S)- 2-methylpyrrolidin-1-yl)methyl)- 1H-benzo[d]imidazol-5- yl)terephthalamide |
| 72 | LQ076- 114 | 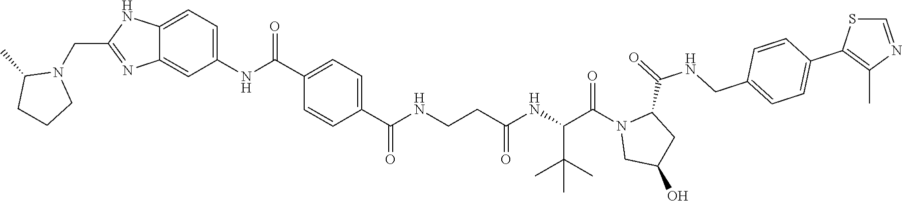 [SMILES: Cc1ncsc1-c1ccc(CNC(=O)[C@@H]2C[C@@H](O)CN2C(=O)[C@@H](NC(=O)CCNC(=O)c2ccc(C(=O)Nc3ccc4[nH]c(CN5CCC[C@@H]5C)nc4c3)cc2)C(C)(C)C)cc1] | N¹1-(3-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2- yl)amino)-3-oxopropyl)-N⁴-(2- (((S)-2-methylpyrrolidin-1- yl)methyl)-1H-benzo[d]imidazol- 5-yl)terephthalamide |
| 73 | LQ076- 115 | 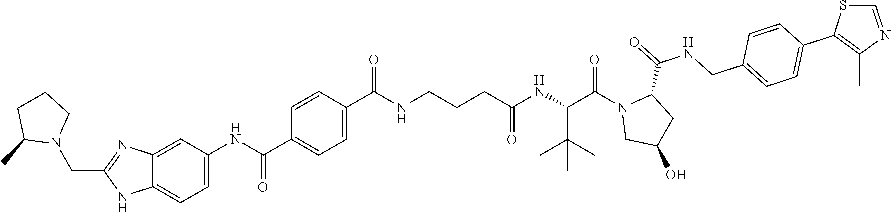 [SMILES: Cc1ncsc1-c1ccc(CNC(=O)[C@@H]2C[C@@H](O)CN2C(=O)[C@@H](NC(=O)CCCNC(=O)c2ccc(C(=O)Nc3ccc4[nH]c(CN5CCC[C@@H]5C)nc4c3)cc2)C(C)(C)C)cc1] | N¹-(4-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2- yl)amino)-4-oxobutyl)-N⁴-(2-(((S)- 2-methylpyrrolidin-1-yl)methyl)- 1H-benzo[d]imidazol-5- yl)terephthalamide |
| 74 | LQ076- 116 | 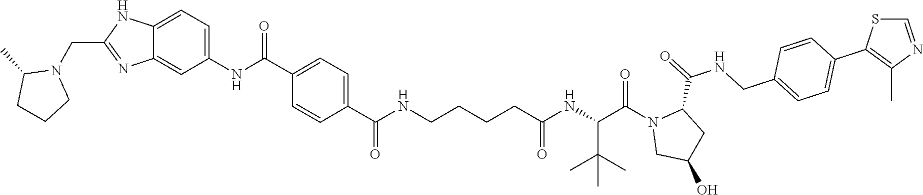 [SMILES: Cc1ncsc1-c1ccc(CNC(=O)[C@@H]2C[C@@H](O)CN2C(=O)[C@@H](NC(=O)CCCCNC(=O)c2ccc(C(=O)Nc3ccc4[nH]c(CN5CCC[C@@H]5C)nc4c3)cc2)C(C)(C)C)cc1] | N¹-(5-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2- yl)amino)-5-oxopentyl)-N⁴-(2-(((S)- 2-methylpyrrolidin-1-yl)methyl)- 1H-benzo[d]imidazol-5- yl)terephthalamide |
| 75 | LQ076- 117 | 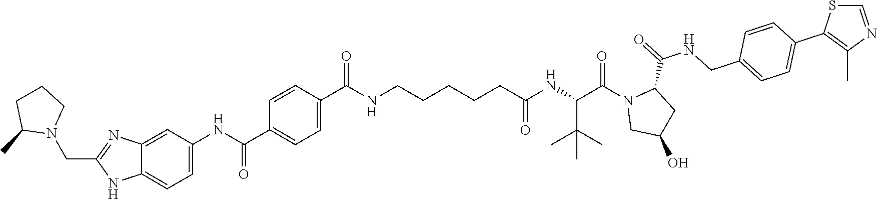 [SMILES: Cc1ncsc1-c1ccc(CNC(=O)[C@@H]2C[C@@H](O)CN2C(=O)[C@@H](NC(=O)CCCCCNC(=O)c2ccc(C(=O)Nc3ccc4[nH]c(CN5CCC[C@@H]5C)nc4c3)cc2)C(C)(C)C)cc1] | N¹-(6-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2-yl) amino)-6-oxohexyl)-N⁴-(2-(((S)-2- methylpyrrolidin-1-yl)methyl)-1H- benzo[d]imidazol-5- yl)terephthalamide |
| 76 | LQ076- 118 | 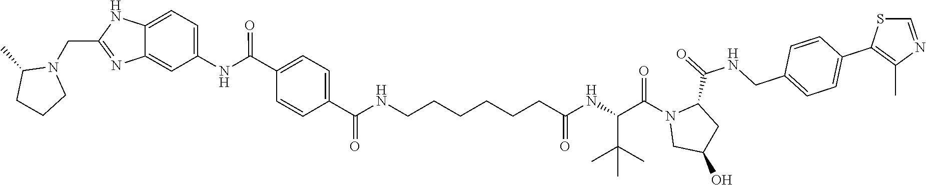 [SMILES: Cc1ncsc1-c1ccc(CNC(=O)[C@@H]2C[C@@H](O)CN2C(=O)[C@@H](NC(=O)CCCCCCNC(=O)c2ccc(C(=O)Nc3ccc4[nH]c(CN5CCC[C@@H]5C)nc4c3)cc2)C(C)(C)C)cc1] | N¹-(7-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2- yl)amino)-7-oxoheptyl)-N⁴-(2-(((S)- 2-methylpyrrolidin-1-yl)methyl)- 1H-benzo[d]imidazol-5- yl)terephthalamide |
| 77 | LQ076- 119 | 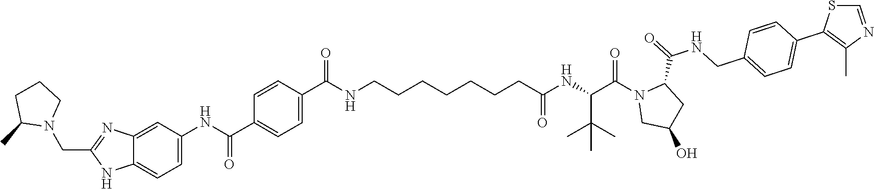 [SMILES: Cc1ncsc1-c1ccc(CNC(=O)[C@@H]2C[C@@H](O)CN2C(=O)[C@@H](NC(=O)CCCCCCCNC(=O)c2ccc(C(=O)Nc3ccc4[nH]c(CN5CCC[C@@H]5C)nc4c3)cc2)C(C)(C)C)cc1] | N¹-(8-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2- yl)amino)-8-oxooctyl)-N⁴-(2-(((S)- 2-methylpyrrolidin-1-yl)methyl)- 1H-benzo[d]imidazol-5- yl)terephthalamide |
| 78 | LQ076- 120 | 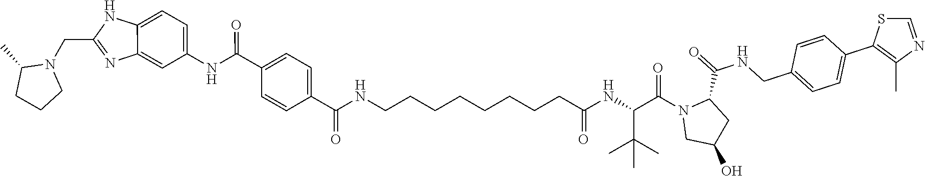 [SMILES: Cc1ncsc1-c1ccc(CNC(=O)[C@@H]2C[C@@H](O)CN2C(=O)[C@@H](NC(=O)CCCCCCCCNC(=O)c2ccc(C(=O)Nc3ccc4[nH]c(CN5CCC[C@@H]5C)nc4c3)cc2)C(C)(C)C)cc1] | N¹-(9-(((S)-1-((2S,4R)-4-hydroxy- 2-((4-(4-methylthiazol-5- y)benzyl)carbamoyl)pyrrolidin-1- yl)-3,3-dimethyl-1-oxobutan-2-yl) amino)-9-oxononyl)-N⁴-(2-(((S)-2- methylpyrrolidin-1-yl)methyl)-1H- benzo[d]imidazol-5- yl)terephthalamide |
| 79 | LQ076- 121 | 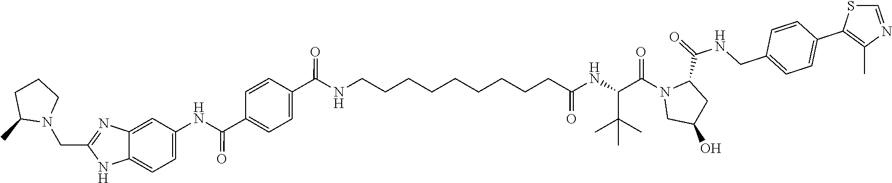 [SMILES: Cc1ncsc1-c1ccc(CNC(=O)[C@@H]2C[C@@H](O)CN2C(=O)[C@@H](NC(=O)CCCCCCCCCNC(=O)c2ccc(C(=O)Nc3ccc4[nH]c(CN5CCC[C@@H]5C)nc4c3)cc2)C(C)(C)C)cc1] | N¹-(10-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2- yl)amino)-10-oxodecyl)-N⁴-(2- (((S)-2-methylpyrrolidin-1- yl)methyl)-1H-benzo[d]imi- dazol-5-yl)terephthalamide |
| 80 | LQ076- 122 | 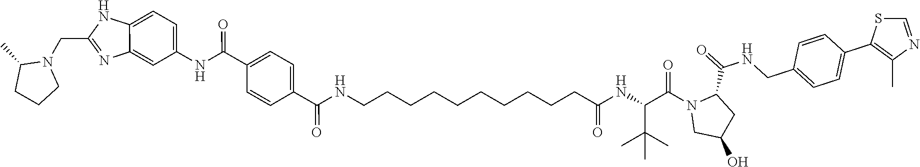 [SMILES: Cc1ncsc1-c1ccc(CNC(=O)[C@@H]2C[C@@H](O)CN2C(=O)[C@@H](NC(=O)CCCCCCCCCCNC(=O)c2ccc(C(=O)Nc3ccc4[nH]c(CN5CCC[C@@H]5C)nc4c3)cc2)C(C)(C)C)cc1] | N¹-(11-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2- yl)amino)-11-oxoundecyl)-N⁴-(2- (((S)-2-methylpyrrolidin-1- yl)methyl)-1H-benzo[d]imidazol-5- yl)terephthalamide |
| 81 | LQ076- 123 | 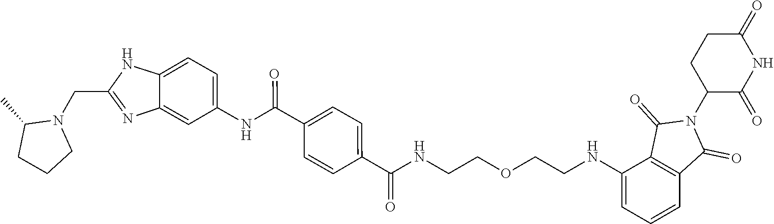 [SMILES: C[C@H]1CCCN1Cc1nc2cc(NC(=O)c3ccc(C(=O)NCCOCCNc4cccc5c4C(=O)N(C4CCC(=O)NC4=O)C5=O)cc3)ccc2[nH]1] | N¹-(2-(2-((2-(2,6-dioxopiperidin-3- yl)-1,3-dioxoisoindolin-4- yl)amino)ethoxy)ethyl)-N⁴-(2-(((S)- 2-methylpyrrolidin-1-yl)methyl)- 1H-benzo[d]imidazol-5- yl)terephthalamide |
| 82 | LQ076- 124 | 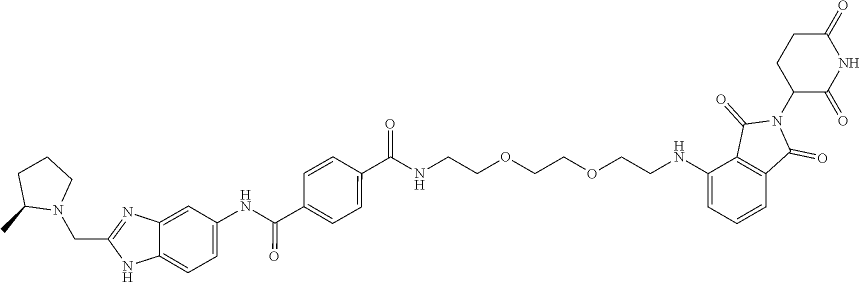 [SMILES: C[C@H]1CCCN1Cc1nc2cc(NC(=O)c3ccc(C(=O)NCCOCCOCCNc4cccc5c4C(=O)N(C4CCC(=O)NC4=O)C5=O)cc3)ccc2[nH]1] | N¹-(2-(2-(2-((2-(2,6-dioxopiperidin- 3-yl)-1,3-dioxoisoindolin-4- yl)amino)ethoxy)ethoxy)ethyl)-N⁴- (2-(((S)-2-methylpyrrolidin-1- yl)methyl)-1H-benzo[d]imidazol-5- yl)terephthalamide |
| 83 | LQ076- 125 | 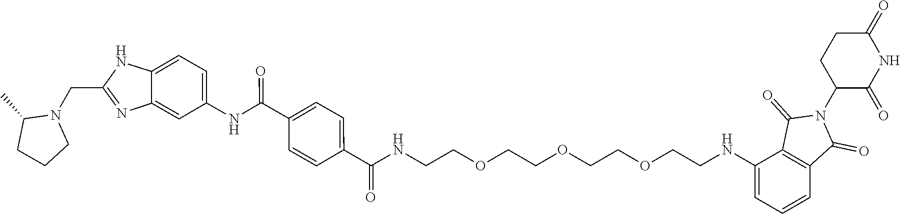 [SMILES: C[C@H]1CCCN1Cc1nc2cc(NC(=O)c3ccc(C(=O)NCCOCCOCCOCCNc4cccc5c4C(=O)N(C4CCC(=O)NC4=O)C5=O)cc3)ccc2[nH]1] | N¹-(2-(2-(2-(2-((2-(2,6- dioxopiperidin-3-yl)-1,3- dioxoisoindolin-4-yl)ami- no)ethoxy)ethoxy)ethoxy)ethyl)- N⁴-(2-(((S)-2-methylpyrrolidin-1- yl)methyl)-1H-benzo[d]imidazol-5- yl)terephthalamide |
| 84 | LQ076- 126 | 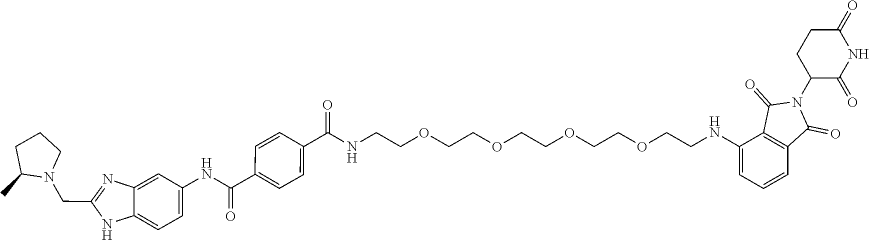 [SMILES: C[C@H]1CCCN1Cc1nc2cc(NC(=O)c3ccc(C(=O)NCCOCCOCCOCCOCCNc4cccc5c4C(=O)N(C4CCC(=O)NC4=O)C5=O)cc3)ccc2[nH]1] | N¹-(14-((2-(2,6-dioxopiperidin-3- yl)-1,3-dioxoisoindolin-4-yl)ami- no)-3,6,9,12-tetraoxatetradecyl)- N⁴-(2-(((S)-2-methylpyrrolidin-1- yl)methyl)-1H-benzo[d]imidazol-5- yl)terephthalamide |
| 85 | LQ076- 127 | 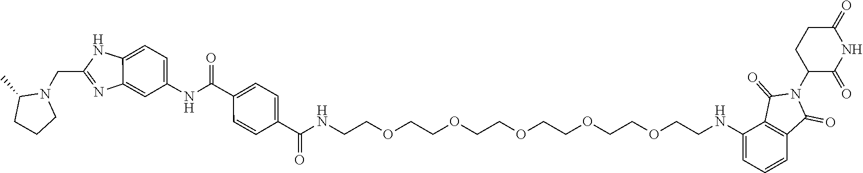 [SMILES: C[C@H]1CCCN1Cc1nc2cc(NC(=O)c3ccc(C(=O)NCCOCCOCCOCCOCCOCCNc4cccc5c4C(=O)N(C4CCC(=O)NC4=O)C5=O)cc3)ccc2[nH]1] | N¹-(17-((2-(2,6-dioxopiperidin-3- yl)-1,3-dioxoisoindolin-4-yl)ami- no)-3,6,9,12,15-pentaoxaheptade- cyl)-N⁴-(2-(((S)-2-methylpyrroli- din-1-yl)methyl)-1H-benzo[d]imi- dazol-5-yl)terephthalamide |
| 86 | LQ076- 128 | 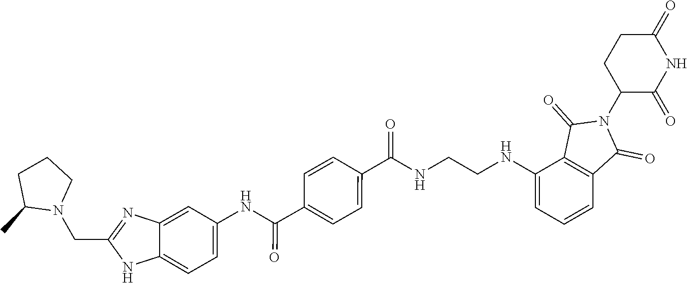 [SMILES: C[C@H]1CCCN1Cc1nc2cc(NC(=O)c3ccc(C(=O)NCCNc4cccc5c4C(=O)N(C4CCC(=O)NC4=O)C5=O)cc3)ccc2[nH]1] | N¹-(2-((2-(2,6-dioxopiperidin-3-yl)- 1,3-dioxoisoindolin-4- yl)amino)ethyl)-N⁴-(2-(((S)-2- methylpyrrolidin-1-yl)methyl)-1H- benzo[d]imidazol-5- yl)terephthalamide |
| 87 | LQ076- 129 | 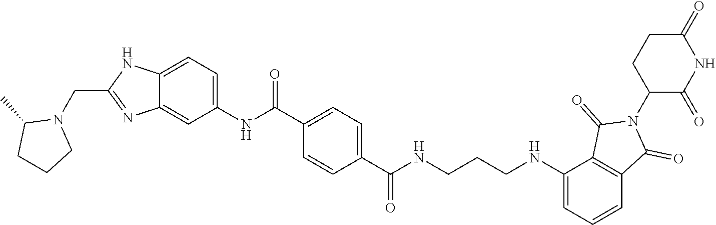 [SMILES: C[C@H]1CCCN1Cc1nc2cc(NC(=O)c3ccc(C(=O)NCCCNc4cccc5c4C(=O)N(C4CCC(=O)NC4=O)C5=O)cc3)ccc2[nH]1] | N¹-(3-((2-(2,6-dioxopipeiridn-3-yl)- 1,3-dioxoisoindolin-4- yl)amino)propyl)-N⁴-(2-(((S)-2- methylpyrrolidin-1-yl)methyl)-1H- benzo[d]imidazol-5- yl)terephthalamide |
| 88 | LQ076- 130 | 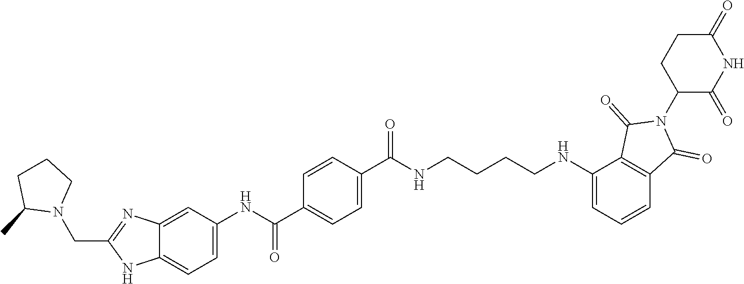 [SMILES: C[C@H]1CCCN1Cc1nc2cc(NC(=O)c3ccc(C(=O)NCCCCNc4cccc5c4C(=O)N(C4CCC(=O)NC4=O)C5=O)cc3)ccc2[nH]1] | N¹-(4-((2-(2,6-dioxopiperidin-3-yl)- 1,3-dioxoisoindolin-4- yl)amino)butyl)-N⁴-(2-(((S)-2- methylpyrrolidin-1-yl)methyl)-1H- benzo[d]imidazol-5- yl)terephthalamide |
| 89 | LQ076- 131 | 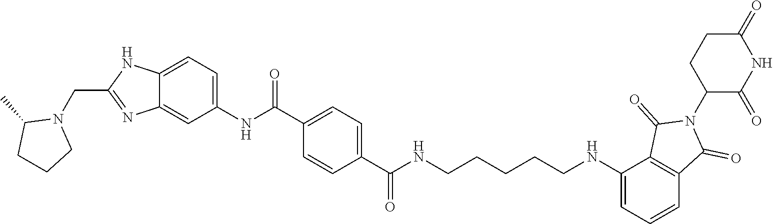 [SMILES: C[C@H]1CCCN1Cc1nc2cc(NC(=O)c3ccc(C(=O)NCCCCCNc4cccc5c4C(=O)N(C4CCC(=O)NC4=O)C5=O)cc3)ccc2[nH]1] | N¹-(5-((2-(2,6-dioxopiperidin-3-yl)- 1,3-dioxoisoindolin-4- yl)amino)pentyl)-N⁴-(2-(((S)-2- methylpyrrolidin-1-yl)methyl)-1H- benzo[d]imidazol-5- yl)terephthalamide |
| 90 | LQ076- 132 | 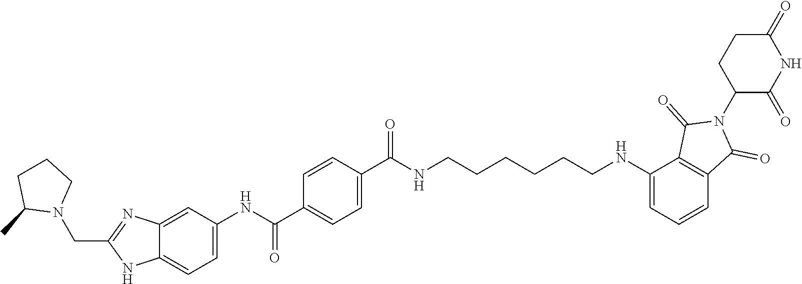 [SMILES: C[C@H]1CCCN1Cc1nc2cc(NC(=O)c3ccc(C(=O)NCCCCCCNc4cccc5c4C(=O)N(C4CCC(=O)NC4=O)C5=O)cc3)ccc2[nH]1] | N¹-(6-((2-(2,6-dioxopiperidin-3-yl)- 1,3-dioxoisoindolin-4- yl)amino)hexyl)-N⁴-(2-(((S)-2- methylpyrrolidin-1-yl)methyl)-1H- benzo[d]imidazol-5- yl)terephthalamide |
| 91 | LQ076- 133 | 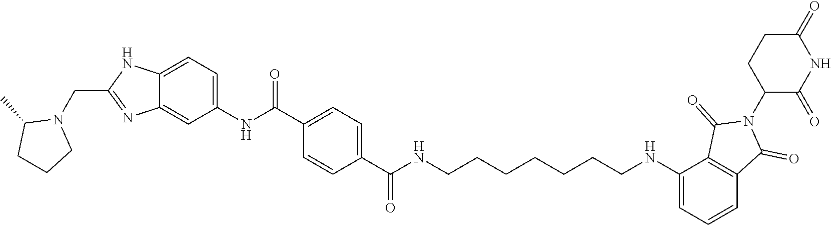 [SMILES: C[C@H]1CCCN1Cc1nc2cc(NC(=O)c3ccc(C(=O)NCCCCCCCNc4cccc5c4C(=O)N(C4CCC(=O)NC4=O)C5=O)cc3)ccc2[nH]1] | N¹-(7-((2-(2,6-dioxopiperidin-3-yl)- 1,3-dioxoisoindolin-4- yl)amino)heptyl)-N⁴-(2-(((S)-2- methylpyrrolidin-1-yl)methyl)-1H- benzo[d]imidazol-5- yl)terephthalamide |
| 92 | LQ076- 134 | 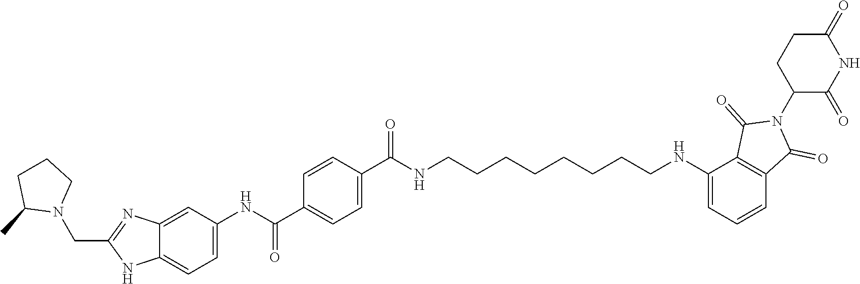 [SMILES: C[C@H]1CCCN1Cc1nc2cc(NC(=O)c3ccc(C(=O)NCCCCCCCCNc4cccc5c4C(=O)N(C4CCC(=O)NC4=O)C5=O)cc3)ccc2[nH]1] | N¹-(8-((2-(2,6-dioxopiperidin-3-yl)- 1,3-dioxoisoindolin-4- yl)amino)octyl)-N⁴-(2-(((S)-2- methylpyrrolidin-1-yl)methyl)-1H- benzo[d]imidazol-5- yl)terephthalamide |
| 94 | LQ076- 135 | 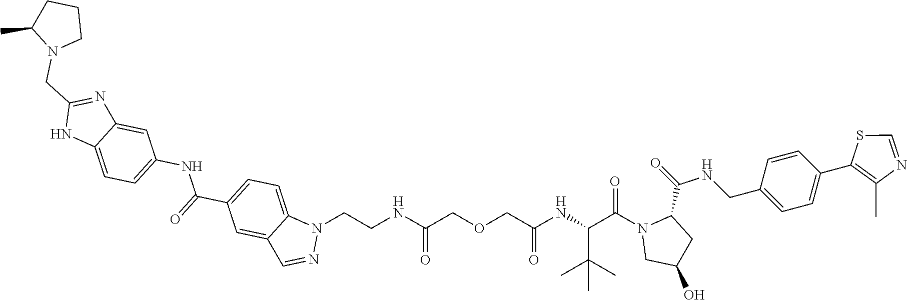 [SMILES: Cc1ncsc1-c1ccc(CNC(=O)[C@@H]2C[C@@H](O)CN2C(=O)[C@@H](NC(=O)COCC(=O)NCCn2ncc3cc(C(=O)Nc4ccc5[nH]c(CN6CCC[C@@H]6C)nc5c4)ccc32)C(C)(C)C)cc1] | 1-(2-(2-(2-(((S)-1-((2S,4R)-4- hydroxy-2-((4-(4-methylthiazol-5- yl)benzyl)carbamoyl)pyrrolidin-1- yl)-3,3-dimethyl-1-oxobutan-2- yl)amino)-2- oxoethoxy)acetamido)ethyl)-N-(2- (((S)-2-methylpyrrolidin-1- yl)methyl)-1H-benzo[d]imidazol-5- yl)-1H-indazole-5-carboxamide |
| 95 | LQ076- 136 | 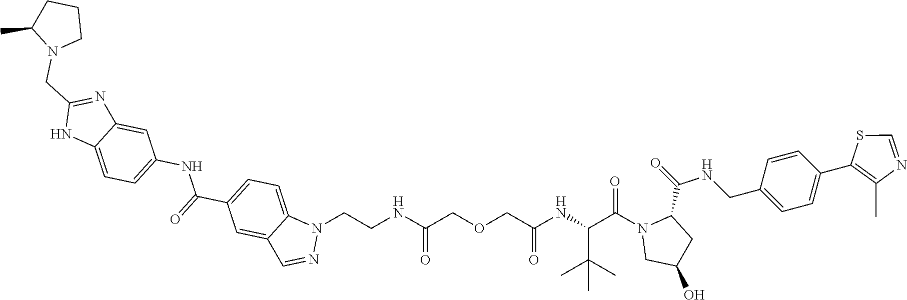 [SMILES: Cc1ncsc1-c1ccc(CNC(=O)[C@@H]2C[C@@H](O)CN2C(=O)[C@@H](NC(=O)COCC(=O)NCCn2ncc3cc(C(=O)Nc4ccc5[nH]c(CN6CCC[C@@H]6C)nc5c4)ccc32)C(C)(C)C)cc1] | 1-(2-(3-(3-(((S)-1-((2S,4R)-4- hydroxy-2-((4-(4-methylthiazol-5- yl)benzyl)carbamoyl)pyrrolidin-1- yl)-3,3-dimethyl-1-oxobutan-2- yl)amino)-3- oxopropoxy)propanamido)ethyl)-N- (2-(((S)-2-methylpyrrolidin-1- yl)methyl)-1H-benzo[d]imidazol-5- yl)-1H-indazole-5-carboxamide |
| 96 | LQ076- 137 | 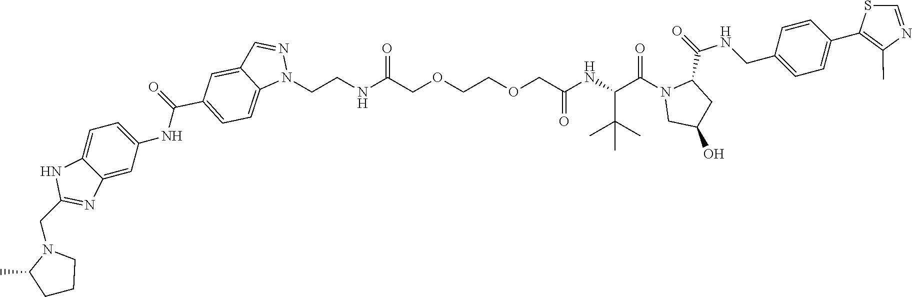 [SMILES: Cc1ncsc1-c1ccc(CNC(=O)[C@@H]2C[C@@H](O)CN2C(=O)[C@@H](NC(=O)COCCOCC(=O)NCCn2ncc3cc(C(=O)Nc4ccc5[nH]c(CN6CCC[C@@H]6C)nc5c4)ccc32)C(C)(C)C)cc1] | 1-((S)-13-((2S,4R)-4-hydroxy-2-((4- (4-methylthiazol-5- yl)benzyl)carbamoyl)pyrrolidine-1- carbonyl)-14,14-dimethyl-4,11- dioxo-6,9-dioxa-3,12- diazapentadecyl)-N-(2-(((S)-2- methylpyrrolidin-1-yl)methyl)-1H- benzo[d]imidazol-5-yl)-1H- indazole-5-carboxamide |
| 97 | LQ076- 138 | 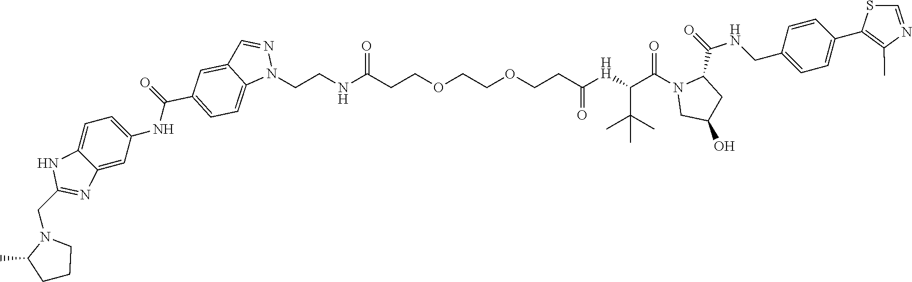 | 1-((S)-15-((2S,4R)-4-hydroxy-2-((4- (4-methylthiazol-5- yl)benzyl)carbamoyl)pyrrolidine-1- carbonyl)-16,16-dimethyl-4,13- dioxo-7,10-dioxa-3,14- diazaheptadecyl)-N-(2-(((S)-2- methylpyrrolidin-1-yl)methyl)-1H- benzo[d]imidazol-5-yl)-1H- indazole-5-carboxamide |
| 98 | LQ076- 139 | 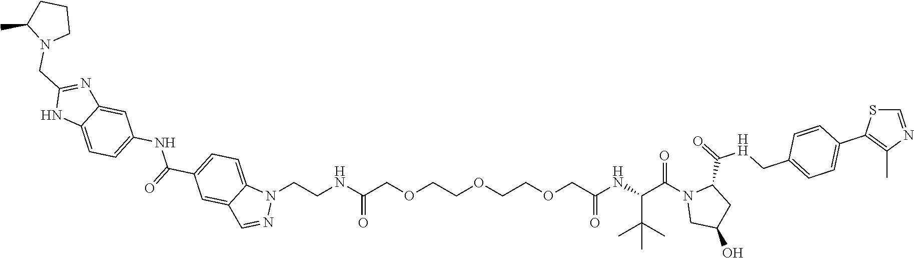 | 1-((S)-16-((2S,4R)-4-hydroxy-2-((4- (4-methylthiazol-5- yl)benzyl)carbamoyl)pyrrolidine-1- carbonyl)-17,17-dimethyl-4,14- dioxo-6,9,12-trioxa-3,15- diazaoctadecyl)-N-(2-(((S)-2- methylpyrrolidin-1-yl)methyl)-1H- benzo[d]imidazol-5-yl)-1H- indazole-5-carboxamide |
| 99 | LQ076- 140 | 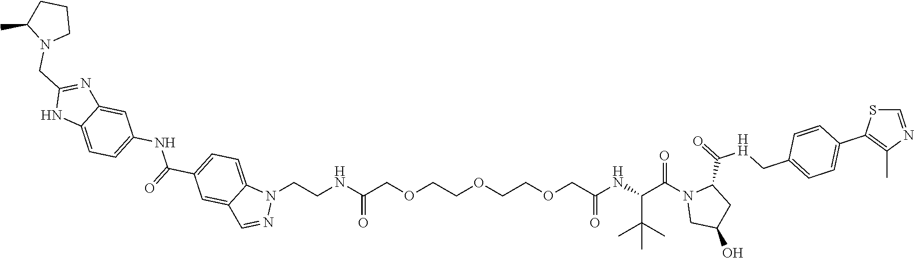 | 1-((S)-18-((2S,4R)-4-hydroxy-2-((4- (4-methylthiazol-5- yl)benzyl)carbamoyl)pyrrolidine-1- carbonyl)-19,19-dimethyl-4,16- dioxo-7,10,13-trioxa-3,17- diazaicosyl)-N-(2-(((S)-2- methylpyrrolidin-1-yl)methyl)-1H- benzo[d]imidazol-5-yl)-1H- indazole-5-carboxamide |
| 100 | LQ076- 141 | 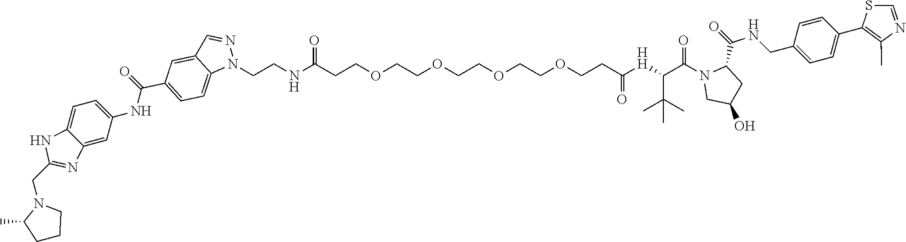 | N¹-((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl)- N¹⁶-(2-(5-((2-(((S)-2- methylpyrrolidin-1-yl)methyl)-1H- benzo[d]imidazol-5-yl)carbamoyl)- 1H-indazol-1-yl)ethyl)-4,7,10,13- tetraoxahexadecanediamide |
| 101 | LQ076- 142 | 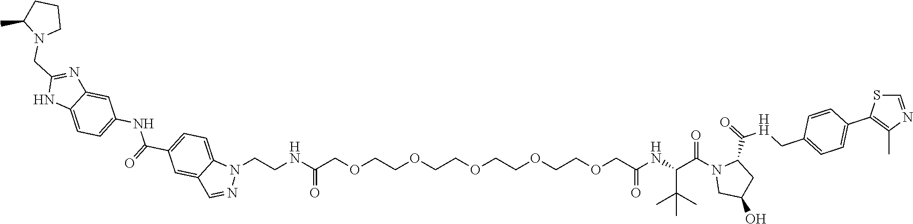 | N¹-((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl)- N¹⁷-(2-(5-((2-(((S)-2- methylpyrrolidin-1-yl)methyl)-1H- benzo[d]imidazol-5-yl)carbamoyl)- 1H-indazol-1-yl)ethyl)-3,6,9,12,15- pentaoxaheptadecanediamide |
| 102 | LQ076- 143 | 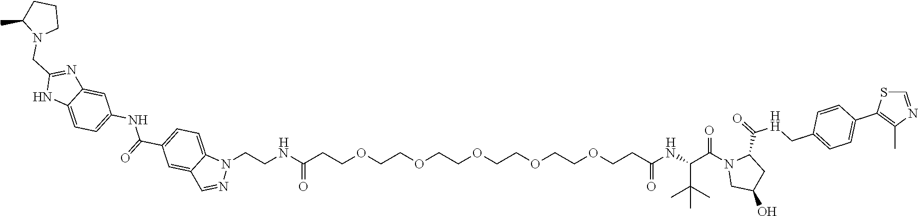 | N¹-((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl)- N¹⁹-(2-(5-((2-(((S)-2- methylpyrrolidin-1-yl)methyl)-1H- benzo[d]imidazol-5-yl)carbamoyl)- 1H-indazol-1-yl)ethyl)-4,7,10,13,16- pentaoxanonadecanediamide |
| 103 | LQ076- 144 | 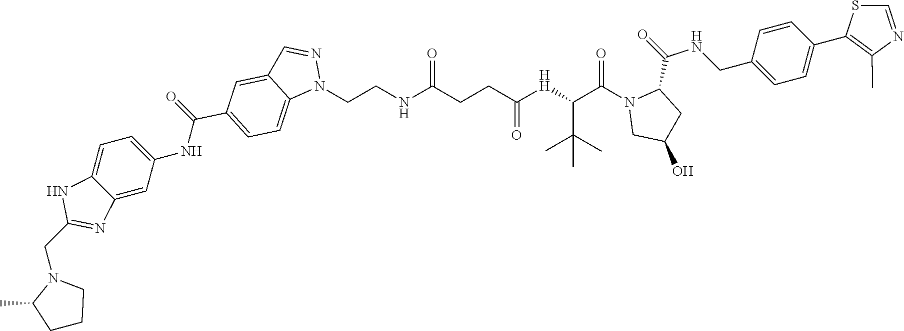 | N¹-((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl)- N⁴-(2-(5-((2-(((S)-2- methylpyrrolidin-1-yl)methyl)-1H- benzo[d]imidazol-5-yl)carbamoyl)- 1H-indazol-1-yl)ethyl)succinamide |
| 104 | LQ076- 145 | 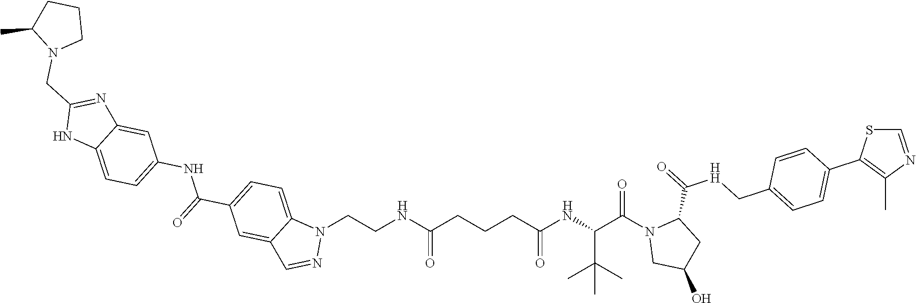 | N¹-((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl)- N⁵-(2-(5-((2-(((S)-2- methylpyrrolidin-1-yl)methyl)-1H- benzo[d]imidazol-5-yl)carbamoyl)- 1H-indazol-1-yl)ethyl)glutaramide |
| 105 | LQ076- 146 | 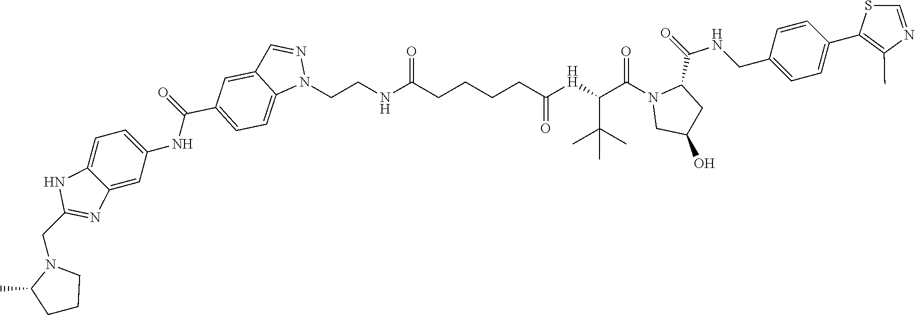 | N¹-((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl)- N⁶-(2-(5-((2-(((S)-2- methylpyrrolidin-1-yl)methyl)-1H- benzo[d]imidazol-5-yl)carbamoyl)- 1H-indazol-1-yl)ethyl)adipamide |
| 106 | LQ076- 147 | 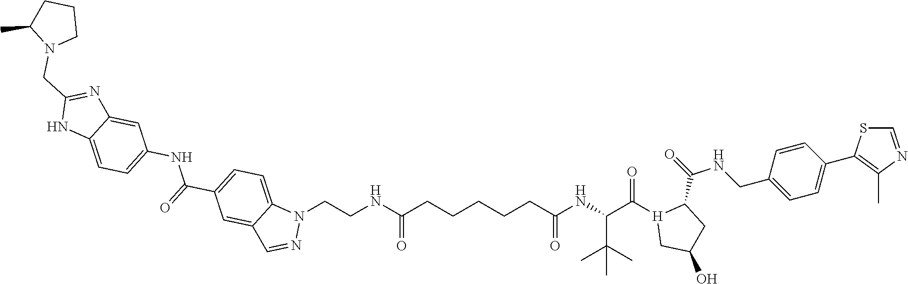 | N¹-((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl)- N⁷-(2-(5-((2-(((S)-2- methylpyrrolidin-1-yl)methyl)-1H- benzo[d]imidazol-5-yl)carbamoyl)- 1H-indazol-1- yl)ethyl)haptanediamide |
| 107 | LQ076- 148 | 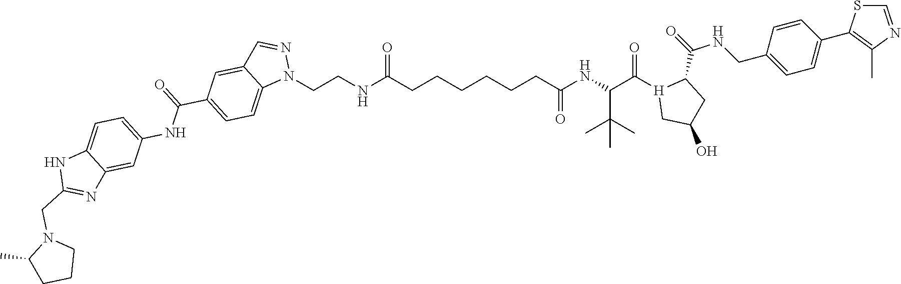 | N¹-((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl)- N⁸-(2-(5-((2-(((S)-2- methylpyrrolidin-1-yl)methyl)-1H- benzo[d]imidazol-5-yl)carbamoyl)- 1H-indazol-1- yl)ethyl)octanediamide |
| 108 | LQ076- 149 | 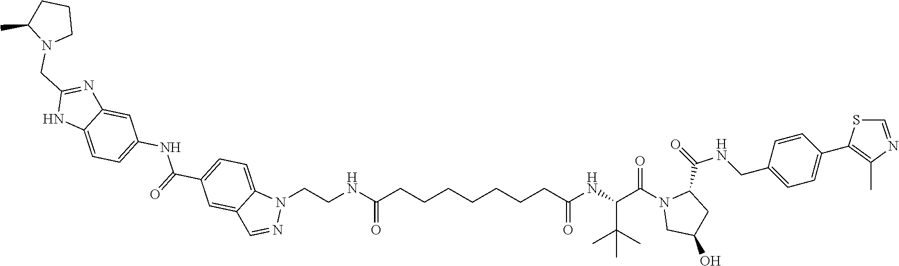 [SMILES: Cc1ncsc1-c1ccc(CNC(=O)[C@@H]2C[C@@H](O)CN2C(=O)[C@@H](NC(=O)CCCCCCCC(=O)NCCn2ncc3cc(C(=O)Nc4ccc5[nH]c(CN6CCC[C@@H]6C)nc5c4)ccc32)C(C)(C)C)cc1] | N¹-((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl)- N⁹-(2-(5-((2-(((S)-2- methylpyrrolidin-1-yl)methyl)-1H- benzo[d]imdiazol-5-yl)carbamoyl)- 1H-indazol-1- yl)ethyl)nonanediamide |
| 109 | LQ076- 150 | 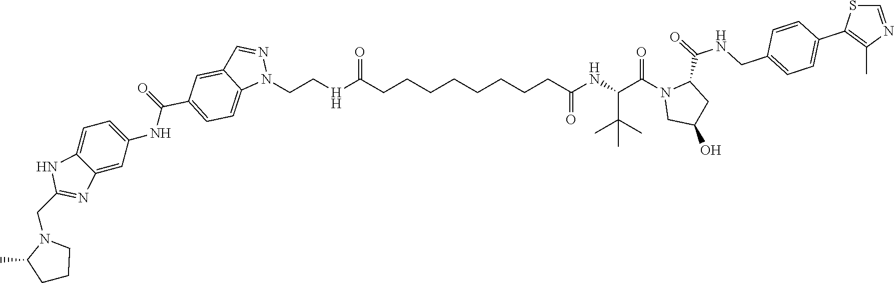 | N¹-((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl)- N¹⁰-(2-(5-((2-(((S)-2- methylpyrrolidin-1-yl)methyl)-1H- benzo[d]imidazol-5-yl)carbamoyl)- 1H-indazol-1- yl)ethyl)decanediamide |
| 110 | LQ076- 151 | 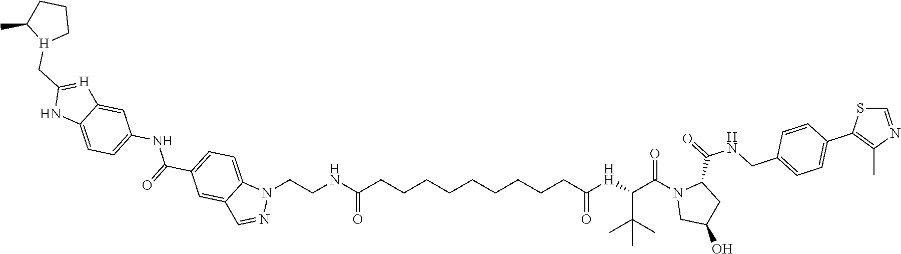 | N¹-((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl)- N¹¹-(2-(5-((2-(((S)-2- methylpyrrolidin-1-yl)methyl)-1H- benzo[d]imidazol-5-yl)carbamoyl)- 1H-indazol-1- yl)ethyl)undecanediamide |
| 111 | LQ076- 152 | 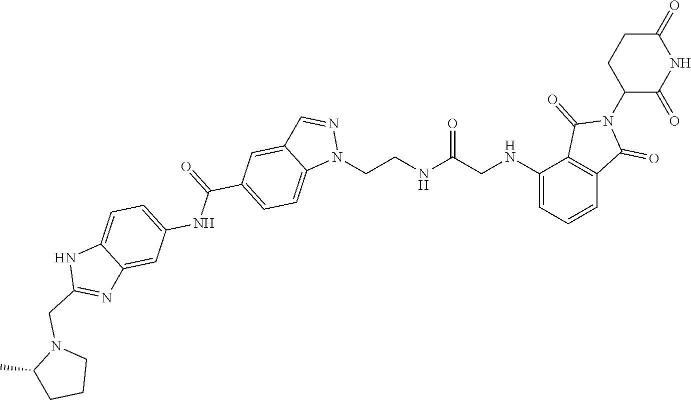 [SMILES: C[C@H]1CCCN1Cc1nc2cc(NC(=O)c3ccc4c(cnn4CCNC(=O)CNc4cccc5c4C(=O)N(C4CCC(=O)NC4=O)C5=O)c3)ccc2[nH]1] | 1-(2-(2-((2-(2,6-dioxopiperidin-3- yl)-1,3-dioxoisoindolin-4- yl)amino)acetamido)ethyl)-N-(2- (((S)-2-methylpyrrolidin-1- yl)methyl)-1H-benzo[d]imidazol-5- yl)-1H-indazole-5-carboxamide |
| 112 | LQ076- 153 | 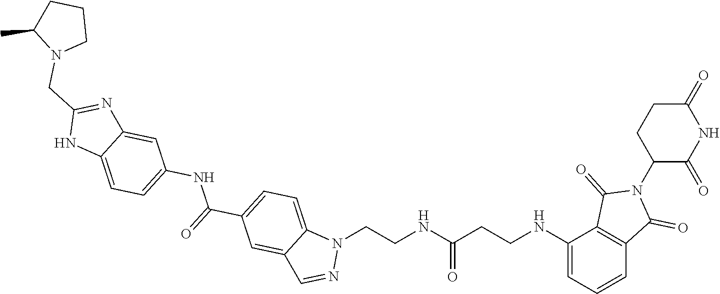 [SMILES: C[C@H]1CCCN1Cc1nc2cc(NC(=O)c3ccc4c(cnn4CCNC(=O)CCNc4cccc5c4C(=O)N(C4CCC(=O)NC4=O)C5=O)c3)ccc2[nH]1] | 1-(2-(3-((2-(2,6-dioxopiperidin-3- yl)-1,3-dioxoisoindolin-4- yl)amino)propanamido)ethyl)-N-(2- (((S)-2-methylpyrrolidin-1- yl)methyl)-1H-benzo[d]imidazol-5- yl)-1H-indazole-5-carboxamide |
| 113 | LQ076- 154 | 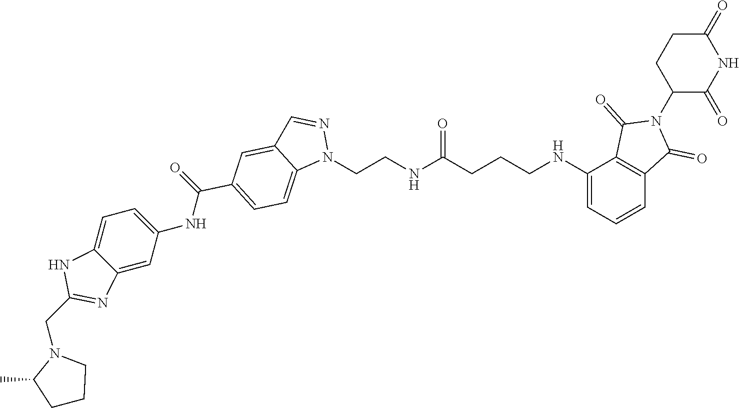 [SMILES: C[C@H]1CCCN1Cc1nc2cc(NC(=O)c3ccc4c(cnn4CCNC(=O)CCCNc4cccc5c4C(=O)N(C4CCC(=O)NC4=O)C5=O)c3)ccc2[nH]1] | 1-(2-(4-((2-(2,6-dioxopiperidin-3- yl)-1,3-dioxoisoindolin-4- yl)amino)butanamido)ethyl)-N-(2- (((S)-2-methylpyrrolidin-1- yl)methyl)-1H-benzo[d]imidazol-5- yl)-1H-indazole-5-carboxamide |
| 114 | LQ076- 155 | 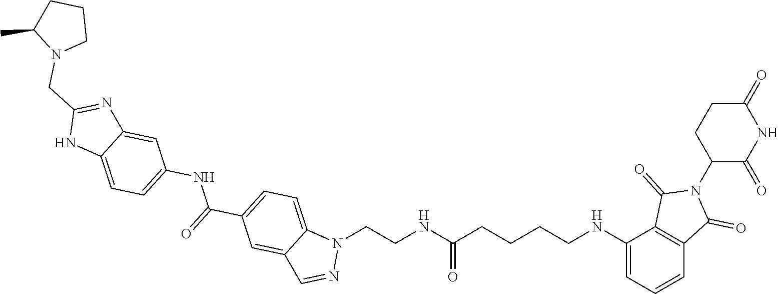 [SMILES: C[C@H]1CCCN1Cc1nc2cc(NC(=O)c3ccc4c(cnn4CCNC(=O)CCCCNc4cccc5c4C(=O)N(C4CCC(=O)NC4=O)C5=O)c3)ccc2[nH]1] | 1-(2-(5-((2-(2,6-dioxopiperidin-3- yl)-1,3-dioxoisoindolin-4- yl)amino)pentanamido)ethyl)-N-(2- (((S)-2-methylpyrrolidin-1- yl)methyl)-1H-benzo[d]imidazol-5- yl)-1H-indazole-5-carboxamide |
| 115 | LQ076- 156 | 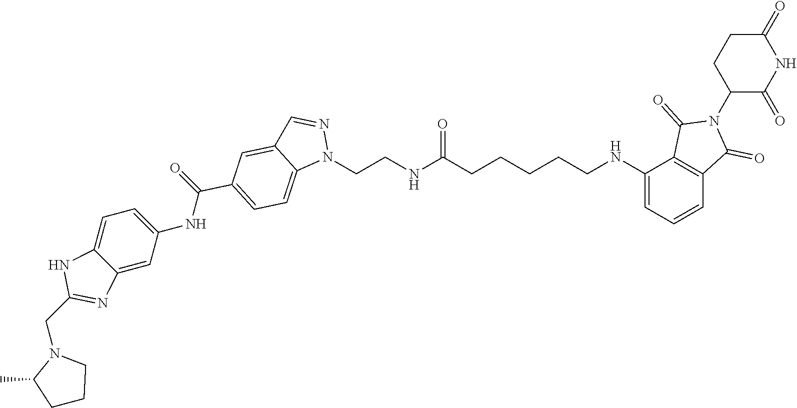 [SMILES: C[C@H]1CCCN1Cc1nc2cc(NC(=O)c3ccc4c(cnn4CCNC(=O)CCCCCNc4cccc5c4C(=O)N(C4CCC(=O)NC4=O)C5=O)c3)ccc2[nH]1] | 1-(2-(6-((2-(2,6-dioxopiperidin-3- yl)-1,3-dioxoisoindolin-4- yl)amino)hexanamido)ethyl)-N-(2- (((S)-2-methylpyrrolidin-1- yl)methyl)-1H-benzo[d]imidazol-5- yl)-1H-indazole-5-carboxamide |
| 116 | LQ076- 157 | 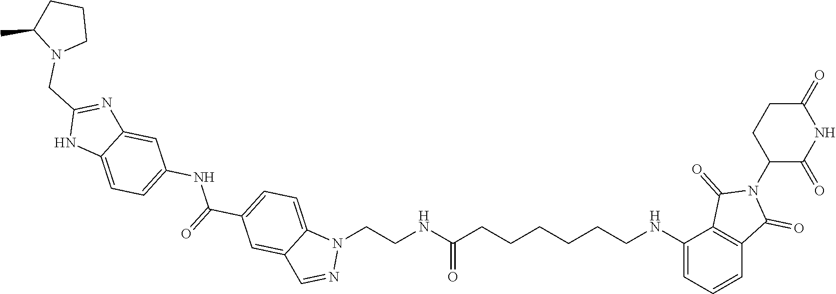 [SMILES: C[C@H]1CCCN1Cc1nc2cc(NC(=O)c3ccc4c(cnn4CCNC(=O)CCCCCCNc4cccc5c4C(=O)N(C4CCC(=O)NC4=O)C5=O)c3)ccc2[nH]1] | 1-(2-(7-((2-(2,6-dioxopiperidin-3- yl)-1,3-dioxoisoindolin-4- yl)amino)heptanamido)ethyl)-N-(2- (((S)-2-methylpyrrolidin-1- y)methyl)-1H-benzo[d]imidazol-5- yl)-1H-indazole-5-carboxamide |
| 117 | LQ076- 158 | 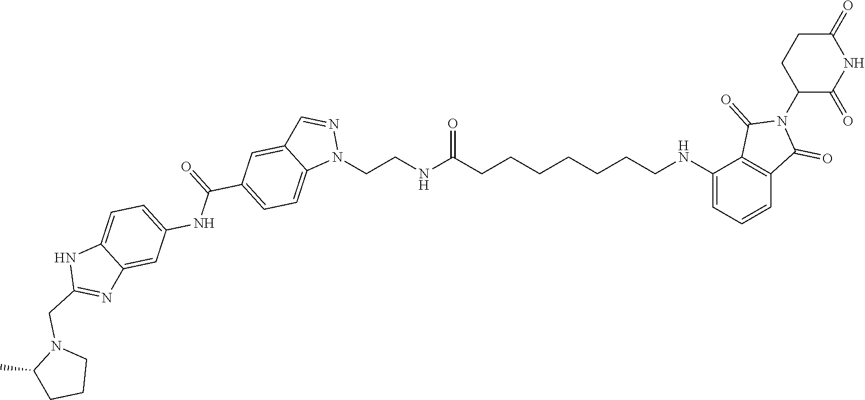 [SMILES: C[C@H]1CCCN1Cc1nc2cc(NC(=O)c3ccc4c(cnn4CCNC(=O)CCCCCCCNc4cccc5c4C(=O)N(C4CCC(=O)NC4=O)C5=O)c3)ccc2[nH]1] | 1-(2-(8-((2-(2,6-dioxopiperidin-3- yl)-1,3-dioxoisoindolin-4- yl)amino)octanamido)ethyl)-N-(2- (((S)-2-methylpyrrolidin-1- yl)methyl)-1H-benzo[d]imidazol-5- yl)-1H-indazole-5-carboxamide |
| 118 | LQ076- 159 | 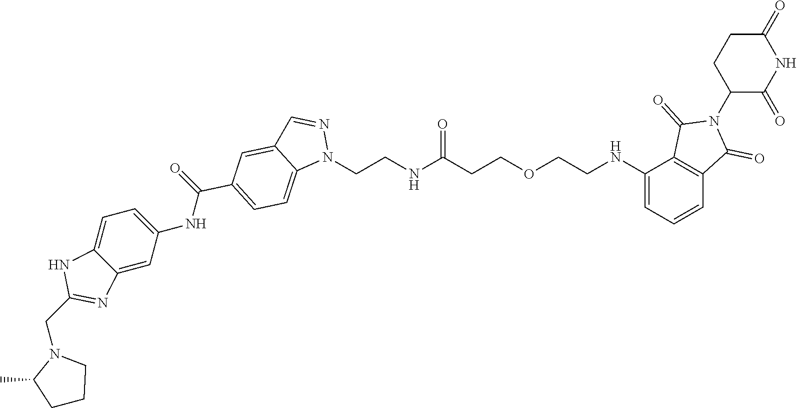 [SMILES: C[C@H]1CCCN1Cc1nc2cc(NC(=O)c3ccc4c(cnn4CCNC(=O)CCOCCNc4cccc5c4C(=O)N(C4CCC(=O)NC4=O)C5=O)c3)ccc2[nH]1] | 1-(2-(3-(2-((2-(2,6-dioxopiperidin- 3-yl)-1,3-dioxoisoindolin-4- yl)amino)ethoxy)propana- mido)ethyl)- N-(2-(((S)-2-methylpyrrolidin-1- yl)methyl)-1H-benzo[d]imidazol-5- yl)-1H-indazole-5-carboxamide |
| 119 | LQ076- 160 | 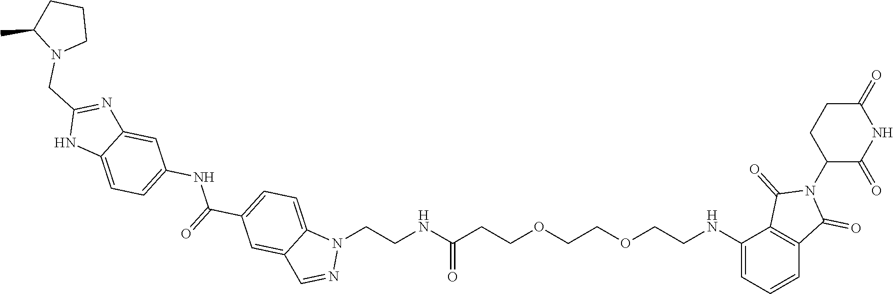 [SMILES: C[C@H]1CCCN1Cc1nc2cc(NC(=O)c3ccc4c(cnn4CCNC(=O)CCOCCOCCNc4cccc5c4C(=O)N(C4CCC(=O)NC4=O)C5=O)c3)ccc2[nH]1] | 1-(2-(3-(2-(2-((2-(2,6- dioxopiperidin- 3-yl)-1,3-dioxoisoindolin-4- yl)amino)ethoxy)ethoxy)propanami- do)ethyl)-N-(2-(((S)-2- methylpyrrolidin-1-yl)methyl)-1H- benzo[d]imidazol-5-yl)-1H- indazole-5-carboxamide |
| 120 | LQ076- 161 | 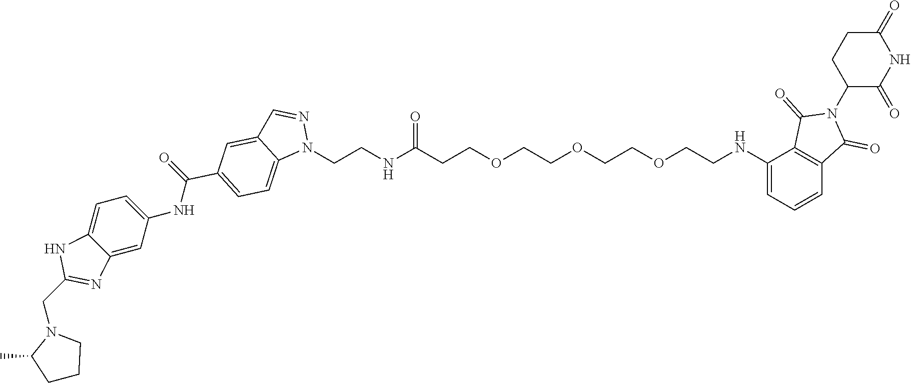 [SMILES: C[C@H]1CCCN1Cc1nc2cc(NC(=O)c3ccc4c(cnn4CCNC(=O)CCOCCOCCOCCNc4cccc5c4C(=O)N(C4CCC(=O)NC4=O)C5=O)c3)ccc2[nH]1] | 1-(1-((2-(2,6-dioxopiperidin-3-yl)- 1,3-dioxoisoindolin-4-yl)amino)-12- oxo-3,6,9-trioxa-13-azapentadecan- 15-y)-N-(2-(((S)-2- methylpyrrolidin-1-yl)methyl)-1H- benzo[d]imidazol-5-yl)-1H- indazole-5-carboxamide |
| 121 | LQ076- 162 | 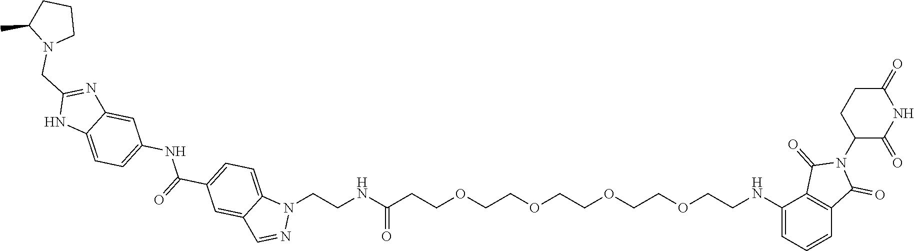 [SMILES: C[C@H]1CCCN1Cc1nc2cc(NC(=O)c3ccc4c(cnn4CCNC(=O)CCOCCOCCOCCOCCNc4cccc5c4C(=O)N(C4CCC(=O)NC4=O)C5=O)c3)ccc2[nH]1] | 1-(1-((2-(2,6-dioxopiperidin-3-yl)- 1,3-dioxoisoindolin-4-yl)amino)-15- oxo-3,6,9,12-tetraoxa-16- azaoctadecan-18-yl)-N-(2-(((S)-2- methylpyrrolidin-1-yl)methyl)-1H- benzo[d]imidazol-5-yl)-1H- indazole-5-carboxamide |
| 122 | LQ076- 163 | 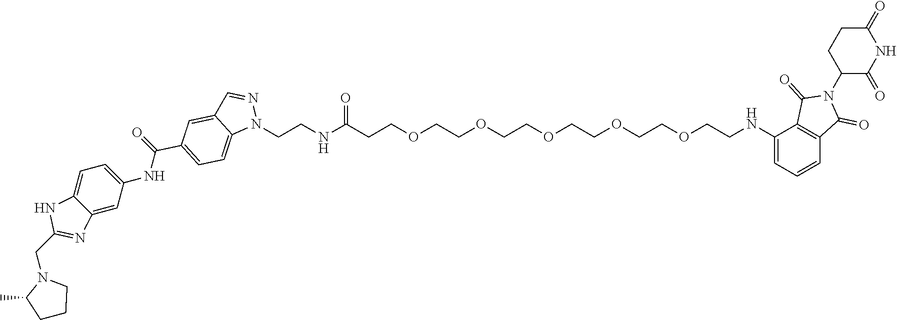 [SMILES: C[C@H]1CCCN1Cc1nc2cc(NC(=O)c3ccc4c(cnn4CCNC(=O)CCOCCOCCOCCOCCOCCNc4cccc5c4C(=O)N(C4CCC(=O)NC4=O)C5=O)c3)ccc2[nH]1] | 1-(1-((2-(2,6-dioxopiperidin-3-yl)- 1,3-dioxoisoindolin-4-yl)amino)-18- oxo-3,6,9,12,15-pentaoxa-19- azahenicosan-21-yl)-N-(2-(((S)-2- methylpyrrolidin-1-yl)methyl)-1H- benzo[d]imidazol-5-yl)-1H- indazole-5-carboxamide |
| 123 | LQ081- 100 | 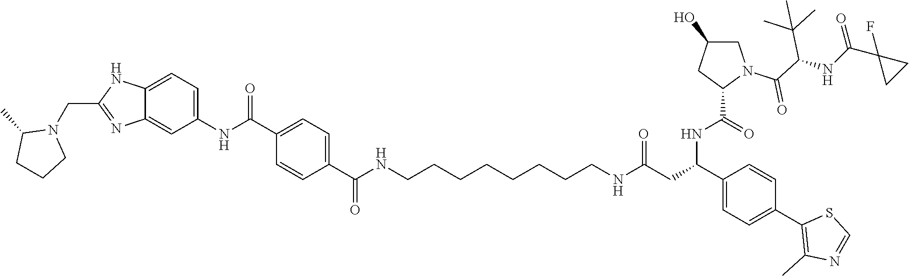 [SMILES: Cc1ncsc1-c1ccc([C@H](CC(=O)NCCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1] | N¹-(8-((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5- yl)phenyl)propanamido)octyl)-N⁴- (2-(((S)-2-methylpyrrolidin-1- yl)methyl)-1H-benzo[d]imidazol-5- yl)terephthalamide |
| 124 | LQ081- 101 | 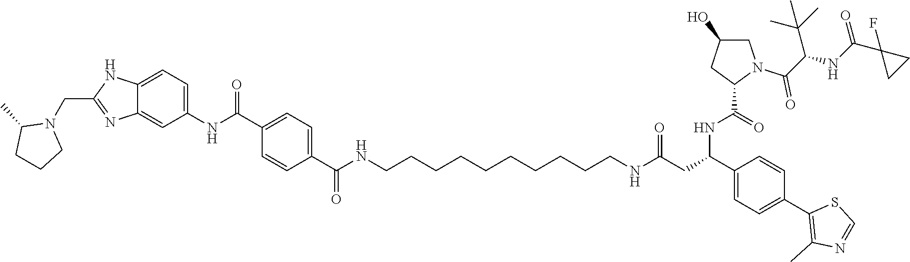 [SMILES: Cc1ncsc1-c1ccc([C@H](CC(=O)NCCCCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1] | N¹-(10-((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5- yl)phenyl)propanamido)decyl)-N⁴- (2-(((S)-2-methylpyrrolidin-1- yl)methyl)-1H-benzo[d]imidazol-5- yl)terephthalamide |
| 125 | LQ081- 102 | 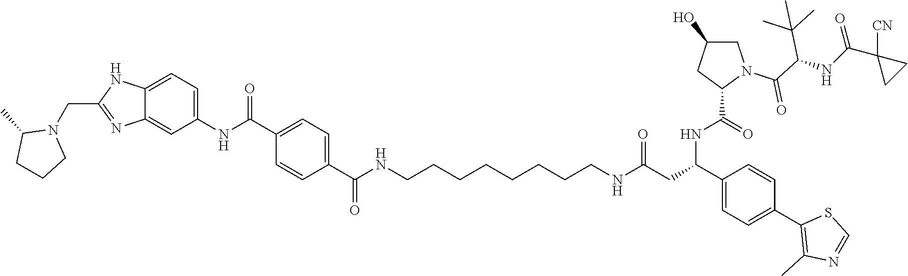 [SMILES: Cc1ncsc1-c1ccc([C@H](CC(=O)NCCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(C#N)CC2)C(C)(C)C)cc1] | N¹-(8-((S)-3-((2S,4R)-1-((S)-2-(1- cyanocyclopropane-1- carboxamido)-3,3- dimethylbutanoyl)-4- hydroxypyrrolidine-2- carboxamido)-3-(4-(4- methylthiazol-5- yl)phenyl)propanamido)octyl)-N⁴- (2-(((S)-2-methylpyrrolidin-1- yl)methyl)-1H-benzo[d]imidazol-5- yl)terephthalamide |
| 126 | LQ081- 103 | 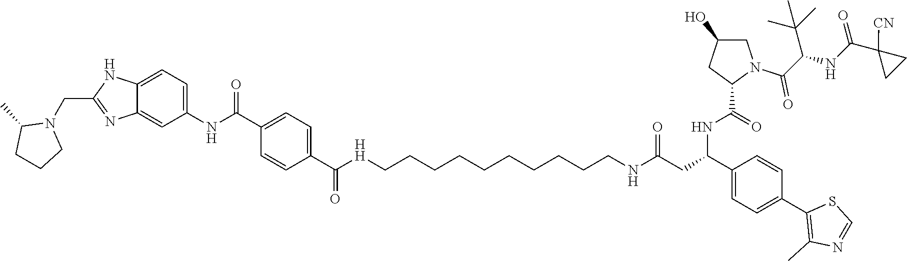 | N¹-(10-((S)-3-((2S,4R)-1-((S)-2-(1- cyanocyclopropane-1- carboxamido)-3,3- dimethylbutanoyl)-4- hydroxypyrrolidine-2- carboxamido)-3-(4-(4- methylthiazol-5- yl)phenyl)propanamido)decyl)-N⁴- (2-(((S)-2-methylpyrrolidin-1- yl)methyl)-1H-benzo[d]imidazol-5- yl)terephthalamide |
| 127 | LQ081- 104 | 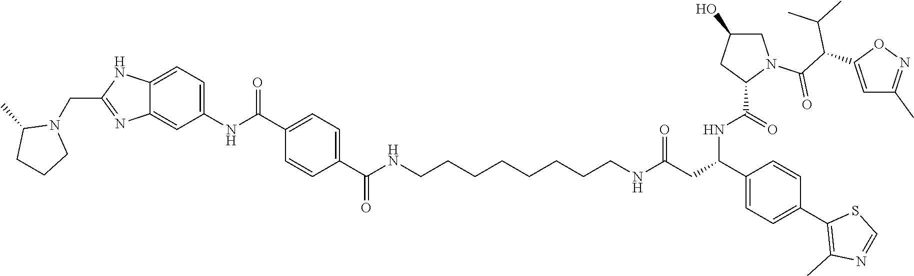 | N¹-(8-((S)-3-((2S,4R)-4-hydroxy-1- ((R)-3-methyl-2-(3-methylisoxazol- 5-yl)butanoyl)pyrrolidine-2- carboxamido)-3-(4-(4- methylthiazol-5- yl)phenyl)propanamido)octyl)-N⁴- (2-(((S)-2-methylpyrrolidin-1- yl)methyl)-1H-benzo[d]imidazol-5- yl)terephthalamide |
| 128 | LQ081- 105 | 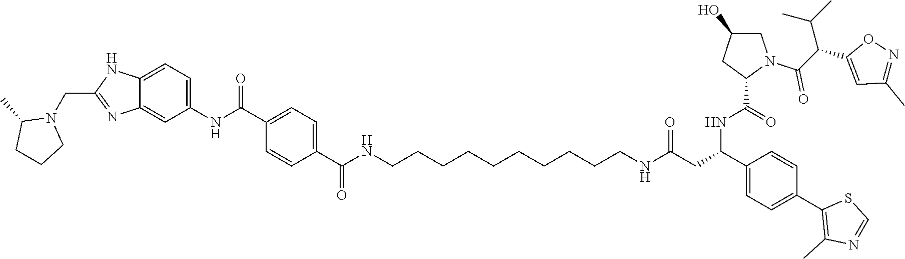 | N¹-(10-((S)-3-((2S,4R)-4-hydroxy- 1-((R)-3-methyl-2-(3-methylisoxa- zol-5-yl)butanoyl)pyrrolidine-2- carboxamido)-3-(4-(4- methylthiazol-5- yl)phenyl)propanamido)decyl)-N⁴- (2-(((S)-2-methylpyrrolidin-1- yl)methyl)-1H-benzo[d]imidazol-5- yl)terephthalamide |
| 131 | LQ081- 108 | 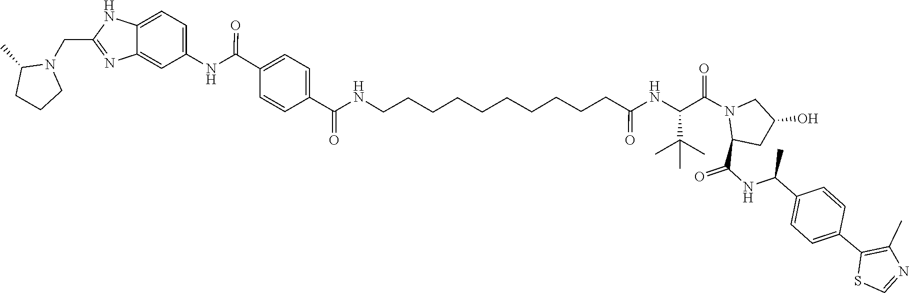 [SMILES: Cc1ncsc1-c1ccc([C@H](C)NC(=O)[C@@H]2C[C@@H](O)CN2C(=O)[C@@H](NC(=O)CCCCCCCCCCNC(=O)c2ccc(C(=O)Nc3ccc4[nH]c(CN5CCC[C@@H]5C)nc4c3)cc2)C(C)(C)C)cc1] | N¹-((11-((S)-1-((2S,4R)-4-hydroxy- 2-(((S)-1-(4-(4-methylthiazol-5- yl)phenyl)ethyl)carba- moyl)pyrrolidin- 1-yl)-3,3-dimethyl-1-oxobutan-2- yl)amino)-11-oxoundecyl)-N⁴-(2- (((S)-2-methylpyrrolidin-1- yl)methyl)-1H-benzo[d]imidazol-5- yll)terephthalamide |
| 132 | LQ081- 109 | 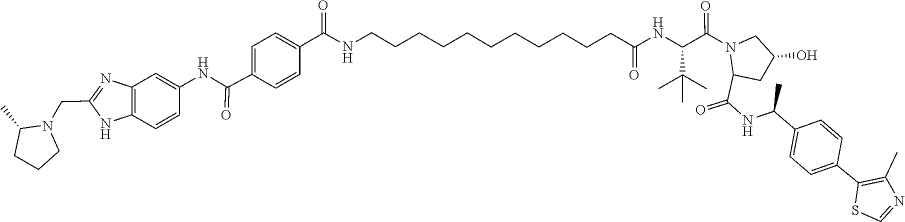 [SMILES: Cc1ncsc1-c1ccc([C@H](C)NC(=O)C2C[C@@H](O)CN2C(=O)[C@@H](NC(=O)CCCCCCCCCCCNC(=O)c2ccc(C(=O)Nc3ccc4[nH]c(CN5CCC[C@@H]5C)nc4c3)cc2)C(C)(C)C)cc1] | N¹-(12-(((S)-1-((2S,4R)-4-hydroxy- 2-(((S)-1-(4-(4-methylthiazol-5- yl)phenyl)ethyl)carba- moyl)pyrrolidin- 1-yl)-3,3-dimethyl-1-oxobutan-2- yl)amino)-12-oxododecyl)-N⁴-(2- (((S)-2-methylpyrrolidin-1- yl)methyl)-1H-benzo[d]imidazol-5- yl)terephthalamide |
| 133 | LQ081- 122 | 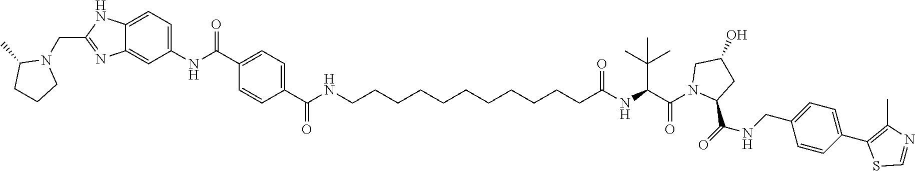 [SMILES: Cc1ncsc1-c1ccc(CNC(=O)[C@@H]2C[C@@H](O)CN2C(=O)[C@@H](NC(=O)CCCCCCCCCCCNC(=O)c2ccc(C(=O)Nc3ccc4[nH]c(CN5CCC[C@@H]5C)nc4c3)cc2)C(C)(C)C)cc1] | N¹-(12-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2- yl)amino)-12-oxododecyl)-N⁴-(2- (((S)-2-methylpyrrolidin-1- yl)methyl)-1H-benzo[d]imidazol-5- yl)terephthalamide |
| 135 | LQ081- 132 | 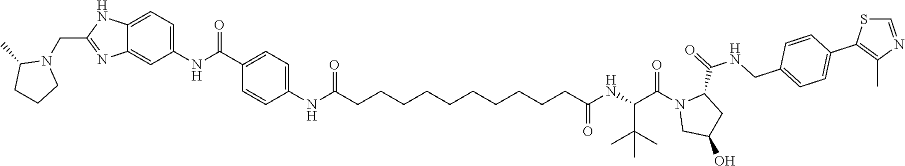 [SMILES: Cc1ncsc1-c1ccc(CNC(=O)[C@@H]2C[C@@H](O)CN2C(=O)[C@@H](NC(=O)CCCCCCCCCCC(=O)Nc2ccc(C(=O)Nc3ccc4[nH]c(CN5CCC[C@@H]5C)nc4c3)cc2)C(C)(C)C)cc1] | N¹-((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl)- N¹²-(4-((2-(((S)-2-methylpyrrolidin- 1-yl)methyl)-1H-benzo[d]imidazol- 5-yl)carba- moyl)phenyl)dodecanediamide |
| 136 | LQ081- 133 | 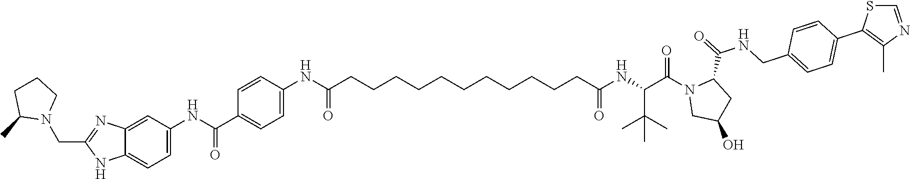 [SMILES: Cc1ncsc1-c1ccc(CNC(=O)[C@@H]2C[C@@H](O)CN2C(=O)[C@@H](NC(=O)CCCCCCCCCCCC(=O)Nc2ccc(C(=O)Nc3ccc4[nH]c(CN5CCC[C@@H]5C)nc4c3)cc2)C(C)(C)C)cc1] | N¹-((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl)- N¹³-(4-((2-(((S)-2-methylpyrrolidin- 1-yl)methyl)-1H-benzo[d]imidazol- 5-yl)carba- moyl)phenyl)tridecanediamide |
| 137 | LQ081- 146 | 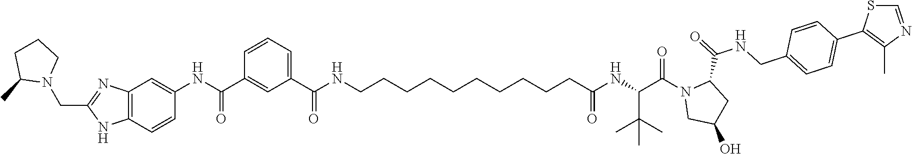 [SMILES: Cc1ncsc1-c1ccc(CNC(=O)[C@@H]2C[C@@H](O)CN2C(=O)[C@@H](NC(=O)CCCCCCCCCCNC(=O)c2cccc(C(=O)Nc3ccc4[nH]c(CN5CCC[C@@H]5C)nc4c3)c2)C(C)(C)C)cc1] | N¹-(11-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2- yl)amino)-11-oxoundecyl)-N³-(2- (((S)-2-methylpyrrolidin-1- yl)methyl)-1H-benzo[d]imidazol-5- yl)isophthalamide |
| 138 | LQ081- 147 | 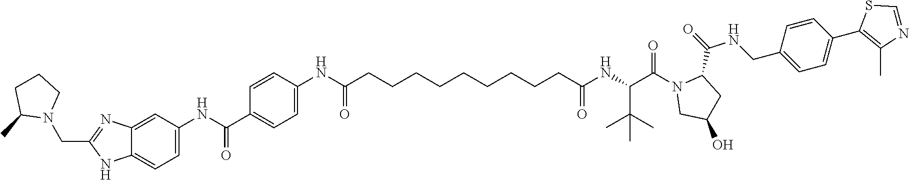 [SMILES: Cc1ncsc1-c1ccc(CNC(=O)[C@@H]2C[C@@H](O)CN2C(=O)[C@@H](NC(=O)CCCCCCCCCC(=O)Nc2ccc(C(=O)Nc3ccc4[nH]c(CN5CCC[C@@H]5C)nc4c3)cc2)C(C)(C)C)cc1] | N¹-((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl)- N¹¹-(4-((2-(((S)-2-methylpyrrolidin- 1-yl)methyl)-1H-benzo[d]imidazol- 5-yl)carba- moyl)phenyl)undecanediamide |
| 139 | LQ081- 150 | 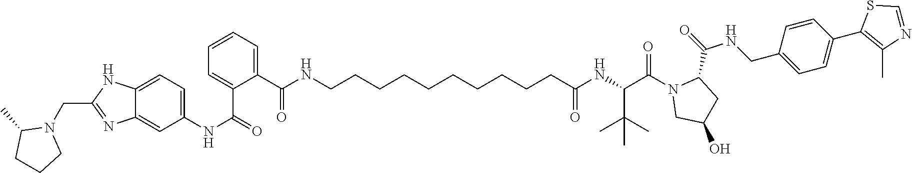 [SMILES: Cc1ncsc1-c1ccc(CNC(=O)[C@@H]2C[C@@H](O)CN2C(=O)[C@@H](NC(=O)CCCCCCCCCCNC(=O)c2ccccc2C(=O)Nc2ccc3[nH]c(CN4CCC[C@@H]4C)nc3c2)C(C)(C)C)cc1] | N¹-((1-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2- yl)amino)-11-oxoundecyl)-N²-(2- (((S)-2-methylpyrrolidin-1- yl)methyl)-1H-benzo[d]imidazol-5- yl)phthalamide |
| 141 | LQ086- 31 | 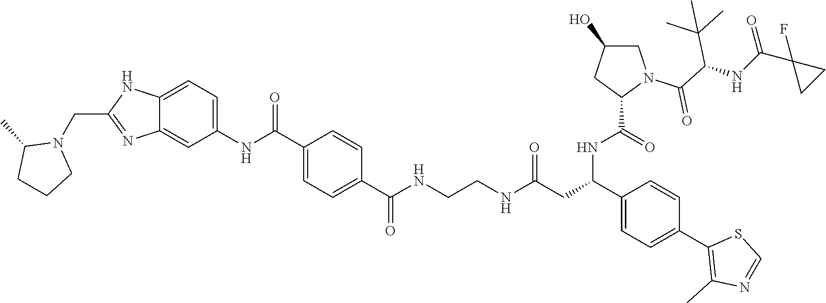 [SMILES: Cc1ncsc1-c1ccc([C@H](CC(=O)NCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1] | N¹-(2-((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5- yl)phenyl)propanamido)ethyl)-N⁴- (2-(((S)-2-methylpyrrolidin-1- yl)methyl)-1H-benzo[d]imidazol-5- yl)terephthalamide |
| 142 | LQ086- 32 | 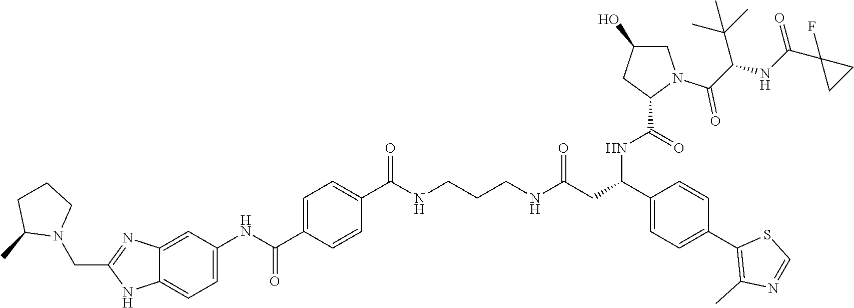 [SMILES: Cc1ncsc1-c1ccc([C@H](CC(=O)NCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1] | N¹-(3-((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5- yl)phenyl)propanamido)propyl)-N⁴- (2-(((S)-2-methylpyrrolidin-1- yl)methyl)-1H-benzo[d]imidazol-5- y)terephthalamide |
| 143 | LQ086- 33 | 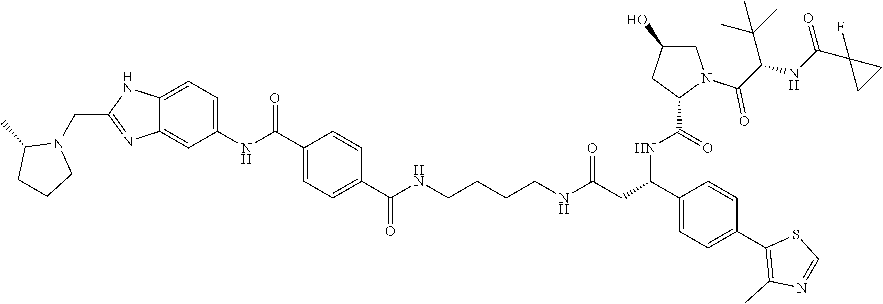 [SMILES: Cc1ncsc1-c1ccc([C@H](CC(=O)NCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1] | N¹-(4-((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5- yl)phenyl)propanamido)butyl)-N⁴- (2-(((S)-2-methylpyrrolidin-1- yl)methyl)-1H-benzo[d]imidazol-5- yl)terephthalamide |
| 144 | LQ086- 34 | 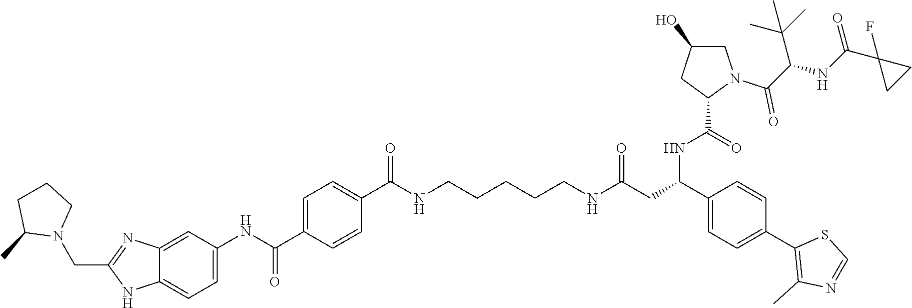 [SMILES: Cc1ncsc1-c1ccc([C@H](CC(=O)NCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1] | N¹-(5-((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5- yl)phenyl)propanamido)pentyl)-N⁴- (2-(((S)-2-methylpyrrolidin-1- yl)methyl)-1H-benzo[d]imidazol-5- yl)terephthalamide |
| 145 | LQ086- 35 | 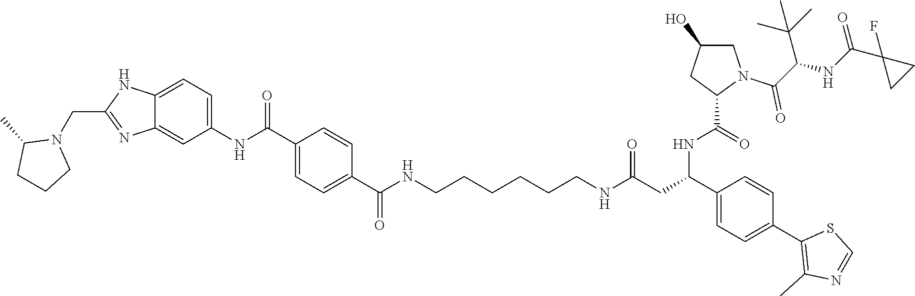 [SMILES: Cc1ncsc1-c1ccc([C@H](CC(=O)NCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1] | N¹-(6-((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5- yl)phenyl)propanamido)hexyl)-N⁴- (2-(((S)-2-methylpyrrolidin-1- yl)methyl)-1H-benzo[d]imidazol-5- yl)terephthalamide |
| 146 | LQ086- 36 | 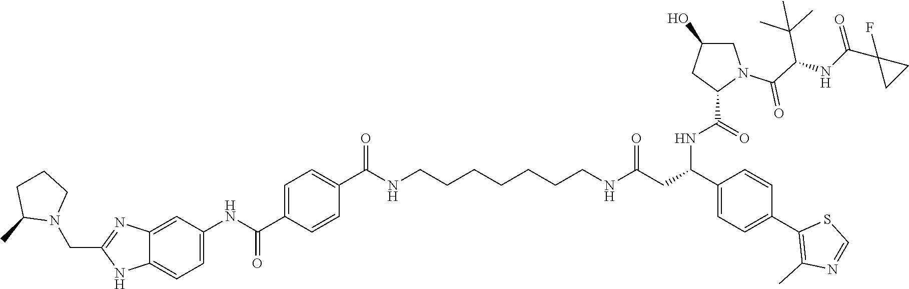 [SMILES: Cc1ncsc1-c1ccc([C@H](CC(=O)NCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1] | N¹-(7-((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5- yl)phenyl)propanamido)heptyl)-N⁴- (2-(((S)-2-methylpyrrolidin-1- yl)methyl)-1H-benzo[d]imidazol-5- yl)terephthalamide |
| 147 | LQ086- 38 | 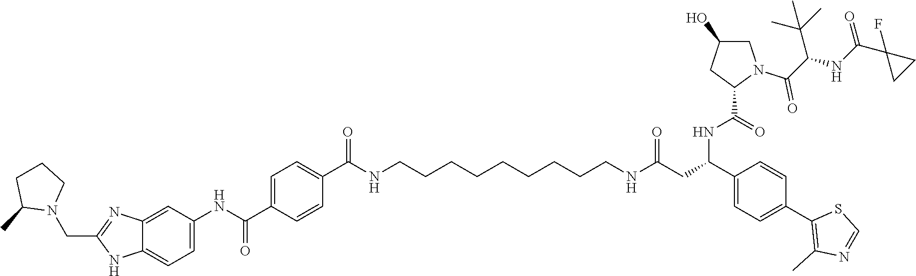 [SMILES: Cc1ncsc1-c1ccc([C@H](CC(=O)NCCCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1] | N¹-(9-((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5- yl)phenyl)propanamido)nonyl)-N⁴- (2-(((S)-2-methylpyrrolidin-1- yl)methyl)-1H-benzo[d]imidazol-5- yl)terephthalamide |
| 148 | LQ086- 40 | 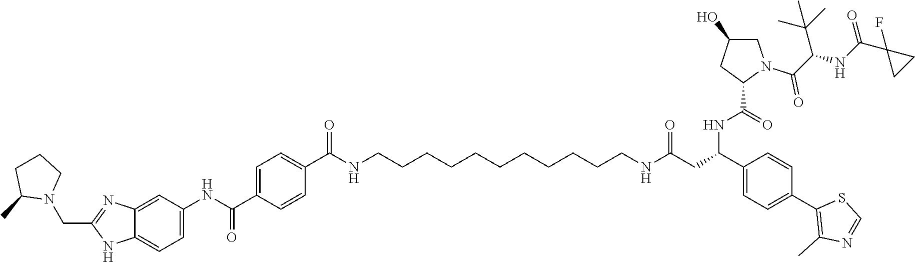 [SMILES: Cc1ncsc1-c1ccc([C@H](CC(=O)NCCCCCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1] | N¹-(11-((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5- yl)phenyl)propanamido)undecyl)- N⁴-(2-(((S)-2-methylpyrrolidin-1- yl)methyl)-1H-benzo[d]imidazol-5- yl)terephthalamide |
| 149 | LQ086- 41 | 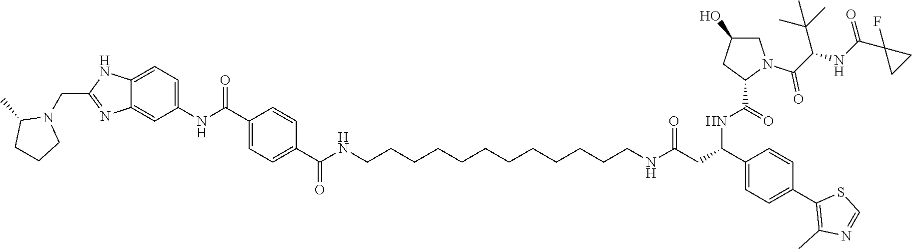 [SMILES: Cc1ncsc1-c1ccc([C@H](CC(=O)NCCCCCCCCCCCCNC(=O)c2ccc(C(=O)Nc3ccc4[nH]c(CN5CCC[C@@H]5C)nc4c3)cc2)NC(=O)[C@@H]2C[C@@H](O)CN2C(=O)[C@@H](NC(=O)C2(F)CC2)C(C)(C)C)cc1] | N¹-(12-((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5- yl)phenyl)propanamido)dodecyl)- N⁴-(2-(((S)-2-methylpyrrolidin-1- yl)methyl)-1H-benzo[d]imidazol-5- yl)terephthalamide |
| 154 | LQ086- 76 | 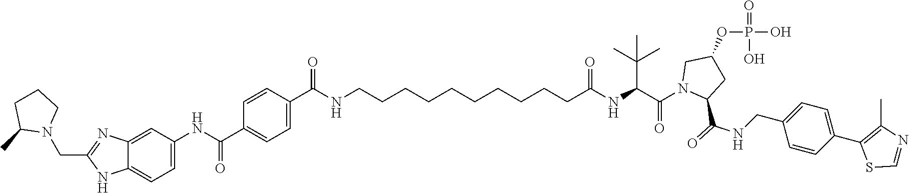 [SMILES: Cc1ncsc1-c1ccc(CNC(=O)[C@@H]2C[C@@H](OP(=O)(O)O)CN2C(=O)[C@@H](NC(=O)CCCCCCCCCCNC(=O)c2ccc(C(=O)Nc3ccc4[nH]c(CN5CCC[C@@H]5C)nc4c3)cc2)C(C)(C)C)cc1] | (3R,5S)-1-((S)-3,3-dimethyl-2-(11- (4-((2-(((S)-2-methylpyrrolidin-1- yl)methyl)-1H-benzo[d]imidazol-5- yl)carbamoyl)benzamido)undecana- mido)butanoyl)-5-((4-(4- methylthiazol-5- yl)benzyl)carbamoyl)pyrrolidin-3-yl dihydrogen phosphate |
| 154 | LQ086- 76Na | 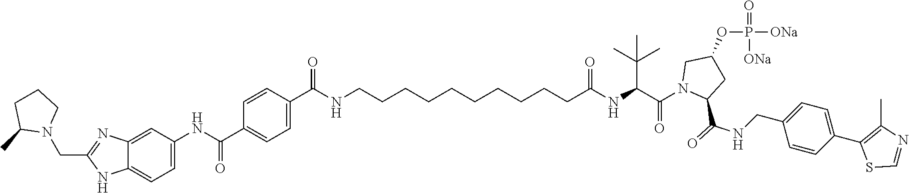 | sodium (3R,5S)-1-((S)-3,3- dimethyl-2-(11-(4-((2-(((S)-2- methylpyrrolidin-1-yl)methyl)-1H- benzo[d]imidazol-5- yl)carbamoyl)benzamido)undecana- mido)butanoyl)-5-((4-(4- methylthiazol-5- yl)benzyl)carbamoyl)pyrrolidin-3-yl phosphate |
| 157 | LQ108- 6 | 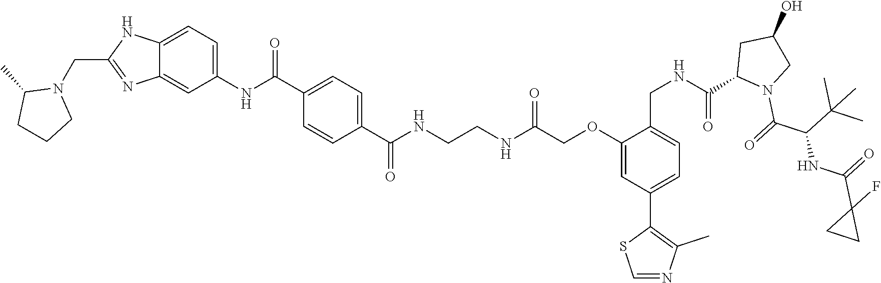 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCNC(=O)c2ccc(C(=O)Nc3ccc4[nH]c(CN5CCC[C@@H]5C)nc4c3)cc2)c1] | N¹-(2-(2-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5- yl)phenoxy)acetamido)ethyl)-N⁴-(2- (((S)-2-methylpyrrolidin-1- yl)methyl)-1H-benzo[d]imidazol-5- yl)terephthalamide |
| 158 | LQ108- 7 | 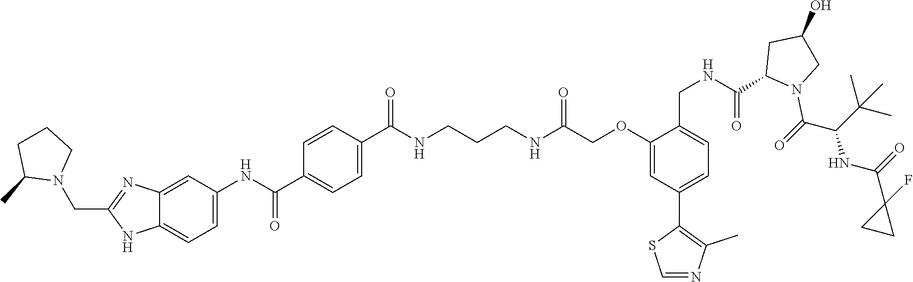 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCNC(=O)c2ccc(C(=O)Nc3ccc4[nH]c(CN5CCC[C@@H]5C)nc4c3)cc2)c1] | N¹-(3-(2-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5- yl)phenoxy)acetamido)propyl)-N⁴- (2-(((S)-2-methylpyrrolidin-1- yl)methyl)-1H-benzo[d]imidazol-5- yl)terephthalamide |
| 159 | LQ108- 8 | 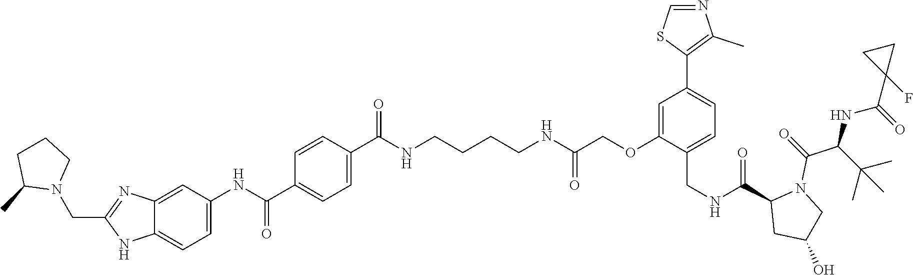 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCNC(=O)c2ccc(C(=O)Nc3ccc4[nH]c(CN5CCC[C@@H]5C)nc4c3)cc2)c1] | N¹-(4-(2-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5- yl)phenoxy)acetamido)butyl)-N⁴-(2- (((S)-2-methylpyrrolidin-1- yl)methyl)-1H-benzo[d]imidazol-5- yl)terephthalamide |
| 160 | LQ108- 9 | 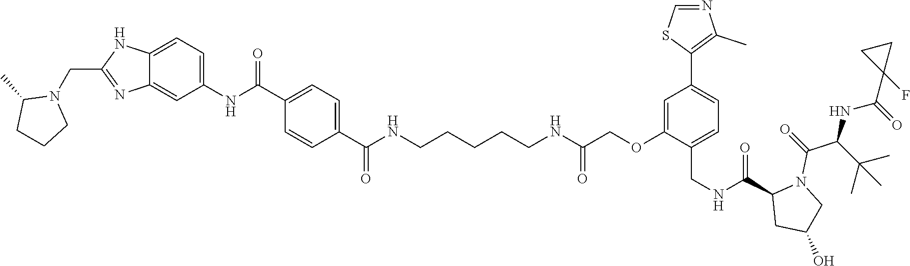 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCNC(=O)c2ccc(C(=O)Nc3ccc4[nH]c(CN5CCC[C@@H]5C)nc4c3)cc2)c1] | N¹-(5-(2-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5- yl)phenoxy)acetamido)pentyl)-N⁴- (2-(((S)-2-methylpyrrolidin-1- yl)methyl)-1H-benzo[d]imidazol-5- yl)terephthalamide |
| 161 | LQ108- 10 | 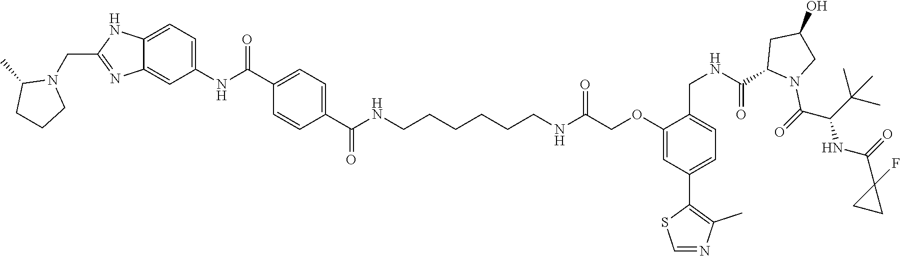 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCNC(=O)c2ccc(C(=O)Nc3ccc4[nH]c(CN5CCC[C@@H]5C)nc4c3)cc2)c1] | N¹-(6-(2-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5- yl)phenoxy)acetamido)hexyl)-N⁴- (2-(((S)-2-methylpyrrolidin-1- yl)methyl)-1H-benzo[d]imidazol-5- yl)terephthalamide |
| 162 | LQ108- 11 | 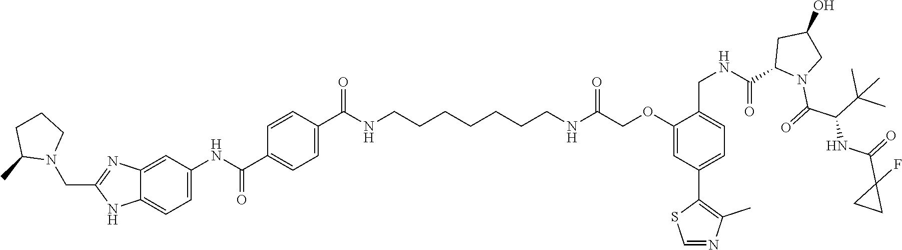 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCCNC(=O)c2ccc(C(=O)Nc3ccc4[nH]c(CN5CCC[C@@H]5C)nc4c3)cc2)c1] | N¹-(7-(2-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5- yl)phenoxy)acetamido)heptyl)-N⁴- (2-(((S)-2-methylpyrrolidin-1- yl)methyl)-1H-benzo[d]imidazol-5- yl)terephthalamide |
| 163 | LQ108- 12 | 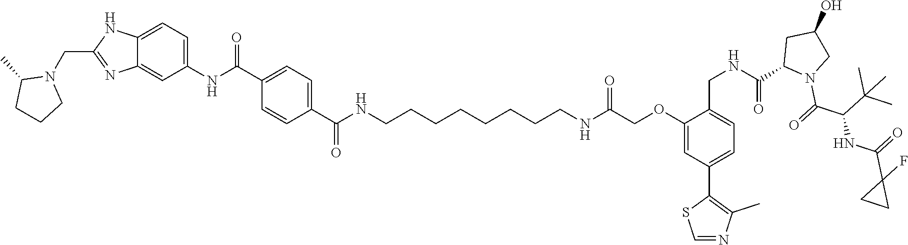 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCCCNC(=O)c2ccc(C(=O)Nc3ccc4[nH]c(CN5CCC[C@@H]5C)nc4c3)cc2)c1] | N¹-(8-(2-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5- yl)phenoxy)acetamido)octyl)-N⁴-(2- (((S)-2-methylpyrrolidin-1- yl)methyl)-1H-benzo[d]imidazol-5- yl)terephthalamide |
| 166 | LQ108- 146 | 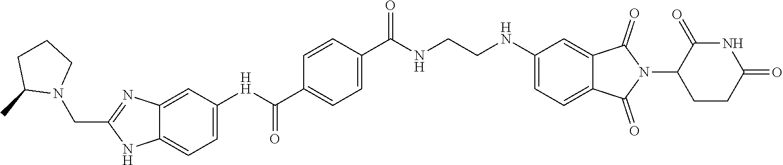 | N¹-(2-((2-(2,6-dioxopiperidin-3-yl)- 1,3-dioxoisoindolin-5- yl)amino)ethyl)-N⁴-(2-(((S)-2- methylpyrrolidin-1-yl)methyl)-1H- benzo[d]imidazol-5- yl)terephthalamide |
| 167 | LQ108- 147 | 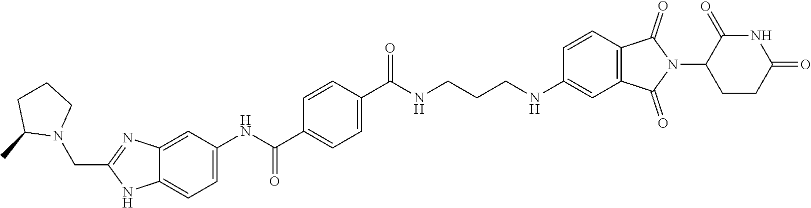 [SMILES: C[C@H]1CCCN1Cc1nc2cc(NC(=O)c3ccc(C(=O)NCCCNc4ccc5c(c4)C(=O)N(C4CCC(=O)NC4=O)C5=O)cc3)ccc2[nH]1] | N¹-(3-((2-(2,6-dioxopiperidin-3-yl)- 1,3-dioxoisoindolin-5- yl)amino)propyl)-N⁴-(2-(((S)-2- methylpyrrolidin-1-yl)methyl)-1H- benzo[d]imidazol-5- yl)terephthalamide |
| 168 | LQ108- 148 | 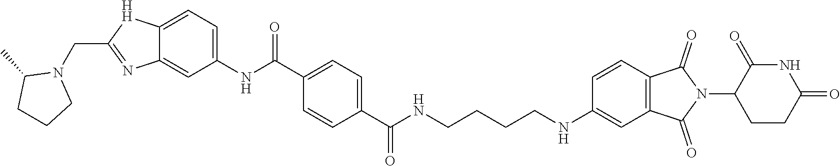 | N¹-(4-((2-(2,6-dioxopiperidin-3-yl)- 1,3-dioxoisoindolin-5- yl)amino)butyl)-N⁴-(2-(((S)-2- methylpyrrolidin-1-yl)methyl)-1H- benzo[d]imidazol-5- yl)terephthalamide |
| 169 | LQ108- 149 | 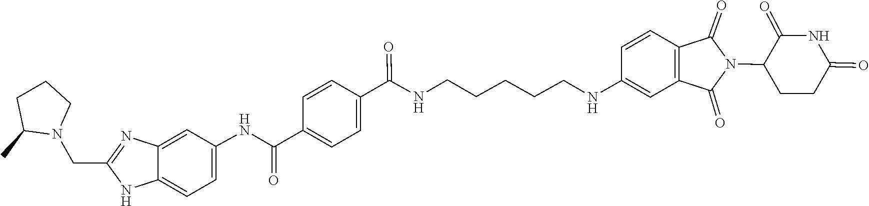 [SMILES: C[C@H]1CCCN1Cc1nc2cc(NC(=O)c3ccc(C(=O)NCCCCCNc4ccc5c(c4)C(=O)N(C4CCC(=O)NC4=O)C5=O)cc3)ccc2[nH]1] | N¹-(5-((2-(2,6-dioxopiperidin-3-yl)- 1,3-dioxoisoindolin-5- yl)amino)pentyl)-N⁴-(2-(((S)-2- methylpyrrolidin-1-yl)methyl)-1H- benzo[d]imidazol-5- yl)terephthalamide |
| 170 | LQ108- 150 | 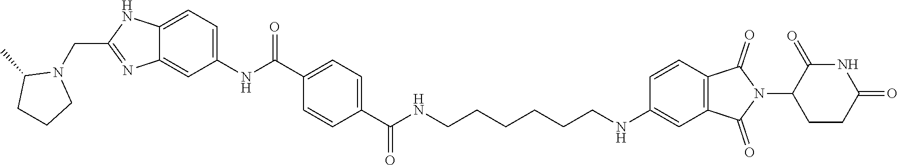 [SMILES: C[C@H]1CCCN1Cc1nc2cc(NC(=O)c3ccc(C(=O)NCCCCCCNc4ccc5c(c4)C(=O)N(C4CCC(=O)NC4=O)C5=O)cc3)ccc2[nH]1] | N¹-(6-((2-(2,6-dioxopiperidin-3-yl)- 1,3-dioxoisoindolin-5- yl)amino)hexyl)-N⁴-(2-(((S)-2- methylpyrrolidin-1-yl)methyl)-1H- benzo[d]imidazol-5- yl)terephthalamide |
| 171 | LQ108- 151 | 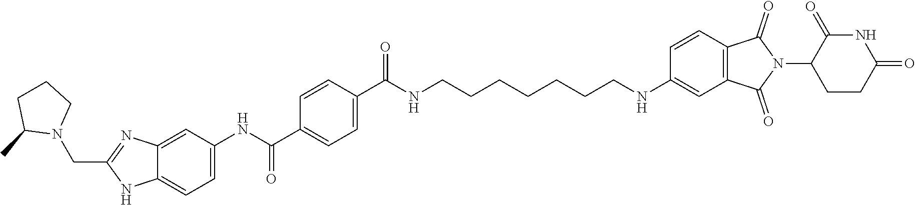 [SMILES: C[C@H]1CCCN1Cc1nc2cc(NC(=O)c3ccc(C(=O)NCCCCCCCNc4ccc5c(c4)C(=O)N(C4CCC(=O)NC4=O)C5=O)cc3)ccc2[nH]1] | N¹-(7-((2-(2,6-dioxopiperidin-3-yl)- 1,3-dioxoisoindolin-5- yl)amino)heptyl)-N⁴-(2-(((S)-2- methylpyrrolidin-1-yl)methyl)-1H- benzo[d]imidazol-5- yl)terephthalamide |
| 172 | LQ108- 152 | 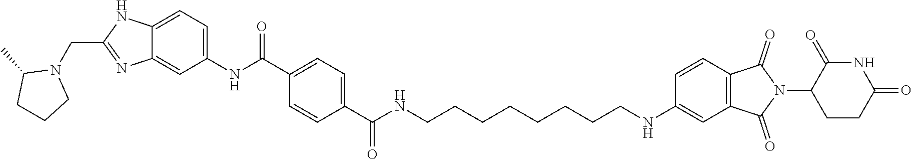 [SMILES: C[C@H]1CCCN1Cc1nc2cc(NC(=O)c3ccc(C(=O)NCCCCCCCCNc4ccc5c(c4)C(=O)N(C4CCC(=O)NC4=O)C5=O)cc3)ccc2[nH]1] | N¹-(8-((2-(2,6-dioxopiperidin-3-yl)- 1,3-dioxoisoindolin-5- yl)amino)octyl)-N⁴-(2-(((S)-2- methylpyrrolidin-1-yl)methyl)-1H- benzo[d]imidazol-5- yl)terephthalamide |
| 173 | LQ108- 153 | 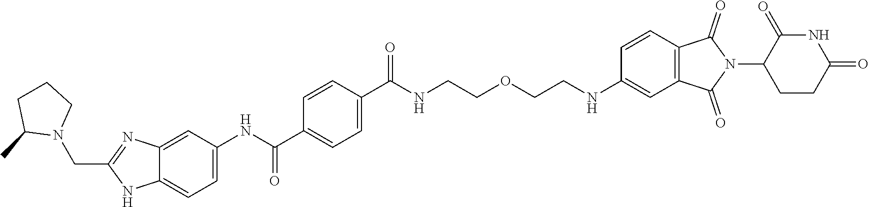 [SMILES: C[C@H]1CCCN1Cc1nc2cc(NC(=O)c3ccc(C(=O)NCCOCCNc4ccc5c(c4)C(=O)N(C4CCC(=O)NC4=O)C5=O)cc3)ccc2[nH]1] | N¹-(2-(2-((2-(2,6-dioxopiperidin-3- yl)-1,3-dioxoisoindolin-5- yl)amino)ethoxy)ethyl)-N⁴-(2-(((S)- 2-methylpyrrolidin-1-yl)methyl)- 1H-benzo[d]imidazol-5- yl)terephthalamide |
| 174 | LQ108- 154 | 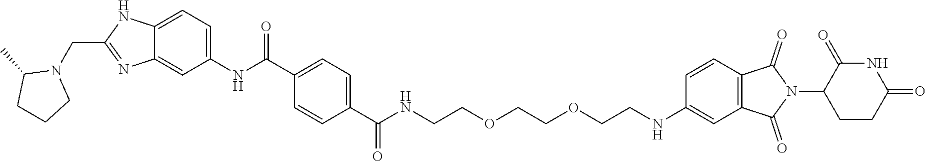 [SMILES: C[C@H]1CCCN1Cc1nc2cc(NC(=O)c3ccc(C(=O)NCCOCCOCCNc4ccc5c(c4)C(=O)N(C4CCC(=O)NC4=O)C5=O)cc3)ccc2[nH]1] | N¹-(2-(2-(2-((2-(2,6-dioxopiperidin- 3-yl)-1,3-dioxoisoindolin-5- yl)amino)ethoxy)ethoxy)ethyl)-N⁴- (2-(((S)-2-methylpyrrolidin-1- yl)methyl)-1H-benzo[d]imidazol-5- yl)terephthalamide |
| 175 | LQ108- 155 | 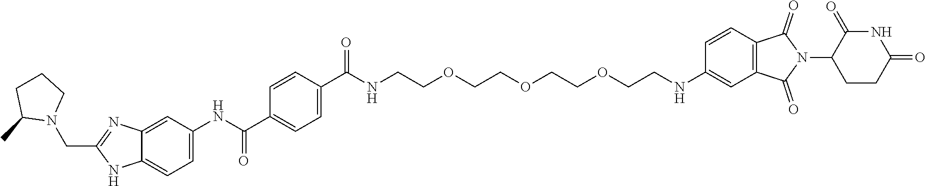 [SMILES: C[C@H]1CCCN1Cc1nc2cc(NC(=O)c3ccc(C(=O)NCCOCCOCCOCCNc4ccc5c(c4)C(=O)N(C4CCC(=O)NC4=O)C5=O)cc3)ccc2[nH]1] | N¹-(2-(2-(2-(2-((2-(2,6- dioxopiperidin-3-yl)-1,3- dioxoisoindolin-5-yl) amino)ethoxy)ethoxy)ethoxy)ethyl)- N⁴-(2-(((S)-2-methylpyrrolidin-1- yl)methyl)-1H-benzo[d]imidazol-5- yl)terephthalamide |
| 176 | LQ108- 156 | 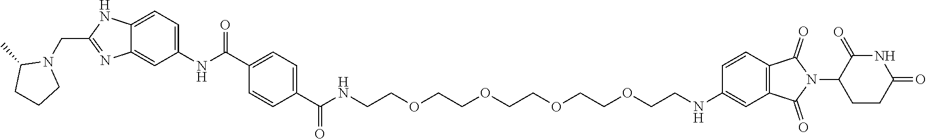 [SMILES: C[C@H]1CCCN1Cc1nc2cc(NC(=O)c3ccc(C(=O)NCCOCCOCCOCCOCCNc4ccc5c(c4)C(=O)N(C4CCC(=O)NC4=O)C5=O)cc3)ccc2[nH]1] | N¹-(14-((2-(2,6-dioxopiperidin- 3-yl)- 1,3-dioxoisoindolin-5-yl)amino)- 3,6,9,12-tetraoxatetradecyl)-N⁴-(2- (((S)-2-methylpyrrolidin-1- yl)methyl)-1H-benzo[d]imidazol-5- yl)terephthalamide |
| 177 | LQ108- 157 | 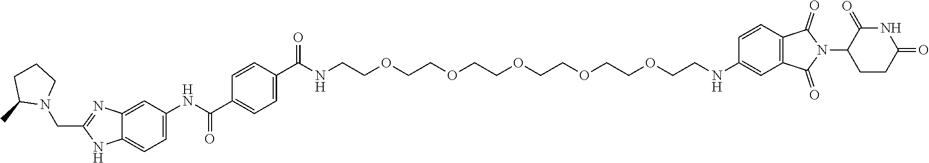 [SMILES: C[C@H]1CCCN1Cc1nc2cc(NC(=O)c3ccc(C(=O)NCCOCCOCCOCCOCCOCCNc4ccc5c(c4)C(=O)N(C4CCC(=O)NC4=O)C5=O)cc3)ccc2[nH]1] | N¹-(17-((2-(2,6-dioxopiperidin- 3-yl)-1,3-dioxoisoindolin- 5-yl)amino)-3,6,9,12,15- pentaoxaheptadecyl)-N⁴- (2-(((S)-2-methylpyrrolidin-1- yl)methyl)-1H-benzo[d]imidazol-5- yl)terephthalamide |
| 178 | LQ118- 23 | 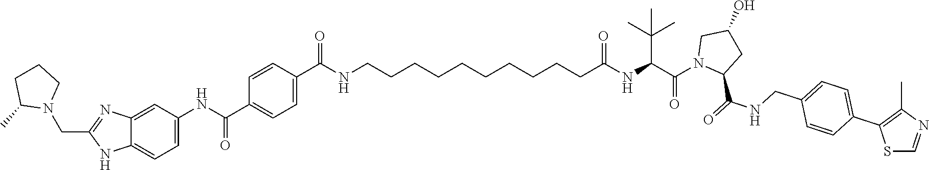 [SMILES: Cc1ncsc1-c1ccc(CNC(=O)[C@@H]2C[C@@H](O)CN2C(=O)[C@@H](NC(=O)CCCCCCCCCCNC(=O)c2ccc(C(=O)Nc3ccc4[nH]c(CN5CCC[C@H]5C)nc4c3)cc2)C(C)(C)C)cc1] | N¹-(11-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2- yl)amino)-11-oxoundecyl)-N⁴-(2- (((R)-2-methylpyrrolidin-1- yl)methyl)-1H-benzo[d]imidazol-5- yl)terephthalamide |
| 179 | LQ118- 24 | 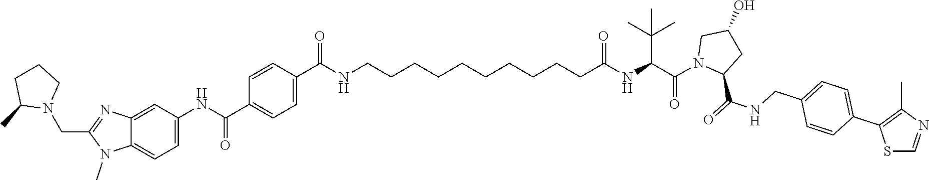 [SMILES: Cc1ncsc1-c1ccc(CNC(=O)[C@@H]2C[C@@H](O)CN2C(=O)[C@@H](NC(=O)CCCCCCCCCCNC(=O)c2ccc(C(=O)Nc3ccc4c(c3)nc(CN3CCC[C@@H]3C)n4C)cc2)C(C)(C)C)cc1] | N¹-(11-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2- yl)amino)-11-oxoundecyl)-N⁴-(1- methyl-2-(((S)-2-methylpyrrolidin- 1-yl)methyl)-1H-benzo[d]imidazol- 5-yl)terephthalamide |
| 180 | LQ118- 25 | 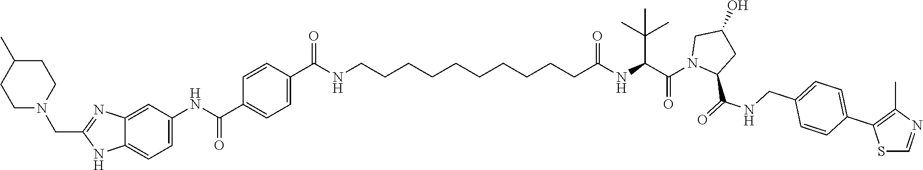 [SMILES: Cc1ncsc1-c1ccc(CNC(=O)[C@@H]2C[C@@H](O)CN2C(=O)[C@@H](NC(=O)CCCCCCCCCCNC(=O)c2ccc(C(=O)Nc3ccc4[nH]c(CN5CCC(C)CC5)nc4c3)cc2)C(C)(C)C)cc1] | N¹-(11-(((S)-1-((2S,4R)-4-hydroxy- 2-((4-(4-methylthiazol-5- yl)benzyl)carbamoyl)pyrrolidin-1- yl)-3,3-dimethyl-1-oxobutan-2- yl)amino)-11-oxoundecyl)-N⁴-(2- ((4-methylpiperidin-1-yl)methyl)- 1H-benzo[d]imidazol-5- yl)terephthalamide |
| 182 | LQ108- 58 | 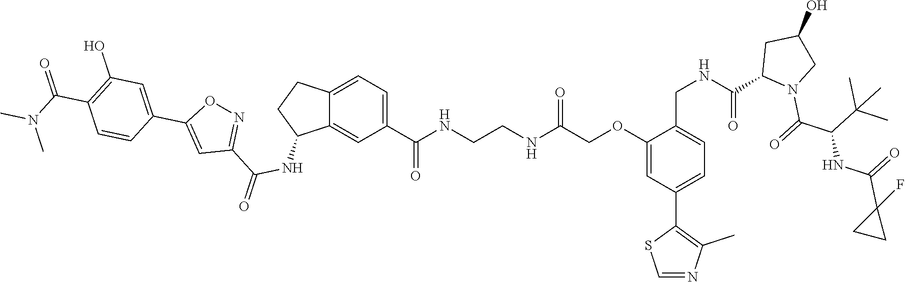 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCNC(=O)c2ccc3c(c2)[C@H](NC(=O)c2cc(-c4ccc(C(=O)N(C)C)c(O)c4)on2)CC3)c1] | 5-(4-(dimethylcarbamoyl)-3- hydroxyphenyl)-N-((R)-6-((2-(2-(2- (((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5- yl)phenoxy)aceta- mido)ethyl)carbamoyl)- 2,3-dihydro-1H-inden-1- yl)isoxazole-3-carboxamide |
| 183 | LQ108- 60 | 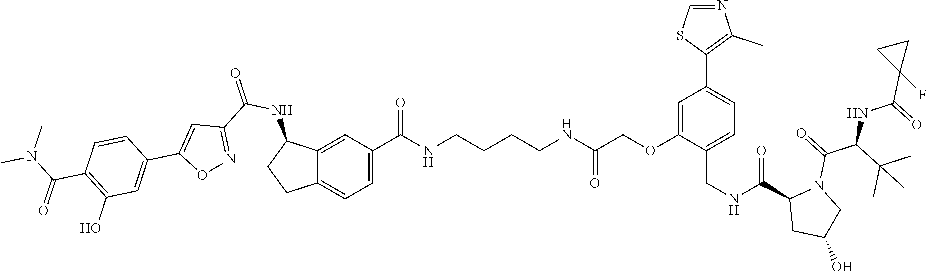 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCNC(=O)c2ccc3c(c2)[C@H](NC(=O)c2cc(-c4ccc(C(=O)N(C)C)c(O)c4)on2)CC3)c1] | 5-(4-(dimethylcarbamoyl)-3- hydroxyphenyl)-N-((R)-6-((4-(2-(2- (((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5- yl)phenoxy)acetamido)butyl)carba- moyl)-2,3-dihydro-1H-inden-1- yl)isoxazole-3-carboxamide |
| 184 | LQ108- 61 | 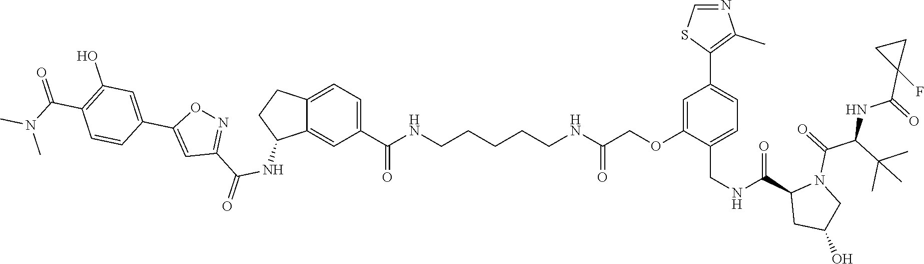 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCNC(=O)c2ccc3c(c2)[C@H](NC(=O)c2cc(-c4ccc(C(=O)N(C)C)c(O)c4)on2)CC3)c1] | 5-(4-(dimethylcarbamoyl)-3- hydroxyphenyl)-N-((R)-6-((5-(2-(2- (((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5-yl) phenoxy)acetamido)pentyl)carba- moyl)-2,3-dihydro-1H-inden-1- yl)isoxazole-3-carboxamide |
| 185 | LQ108- 62 | 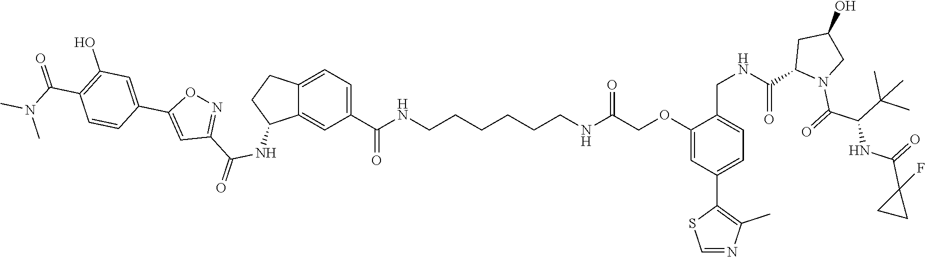 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCNC(=O)c2ccc3c(c2)[C@H](NC(=O)c2cc(-c4ccc(C(=O)N(C)C)c(O)c4)on2)CC3)c1] | 5-(4-(dimethylcarbamoyl)-3- hydroxyphenyl)-N-((R)-6-((6-(2- (2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5- yl)phenoxy)acetamido)hexyl)carba- moyl)-2,3-dihydro-1H-inden-1- yl)isoxazole-3-carboxamide |
| 186 | LQ108- 63 | 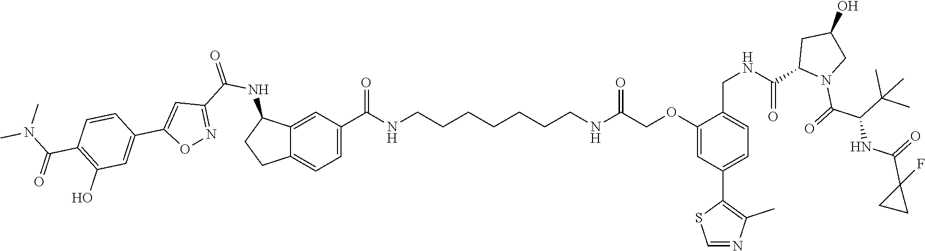 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCCNC(=O)c2ccc3c(c2)[C@H](NC(=O)c2cc(-c4ccc(C(=O)N(C)C)c(O)c4)on2)CC3)c1] | 5-(4-(dimethylcarbamoyl)-3- hydroxyphenyl)-N-((R)-6-((7-(2-(2- (((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5-yl) phenoxy)acetamido)heptyl)carba- moyl)-2,3-dihydro-1H-inden-1- yl)isoxazole-3-carboxamide |
| 187 | LQ108- 64 | 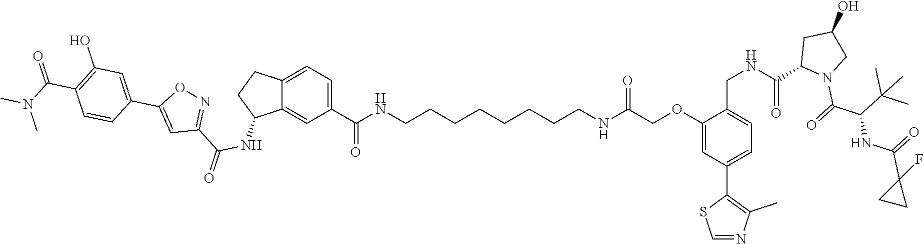 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCCCNC(=O)c2ccc3c(c2)[C@H](NC(=O)c2cc(-c4ccc(C(=O)N(C)C)c(O)c4)on2)CC3)c1] | 5-(4-(dimethylcarbamoyl)-3- hydroxyphenyl)-N-((R)-6-((8-(2-(2- (((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5- yl)phenoxy)acetami- do)octyl)carbamoyl)- 2,3-dihydro-1H-inden-1- yl)isoxazole-3-carboxamide |
| 188 | LQ108- 65 | 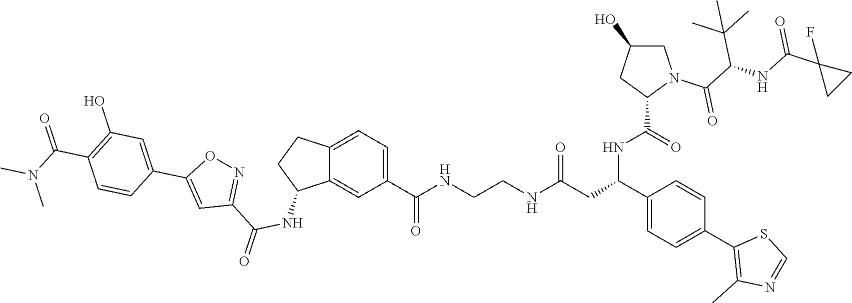 [SMILES: Cc1ncsc1-c1ccc([C@H](CC(=O)NCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-((2- ((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5- y)phenyl)propanamido)ethyl)carba- moyl)-2,3-dihydro-1H-inden-1- yl)isoxazole-3-carboxamide |
| 189 | LQ108- 66 | 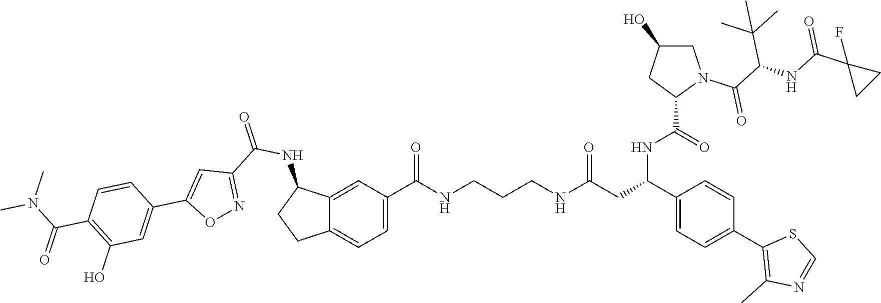 [SMILES: Cc1ncsc1-c1ccc([C@H](CC(=O)NCCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-((3-((S)- 3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5-yl) phenyl)propanamido)propyl)carba- moyl)-2,3-dihydro-1H-inden-1- yl)isoxazole-3-carboxamide |
| 190 | LQ108- 67 | 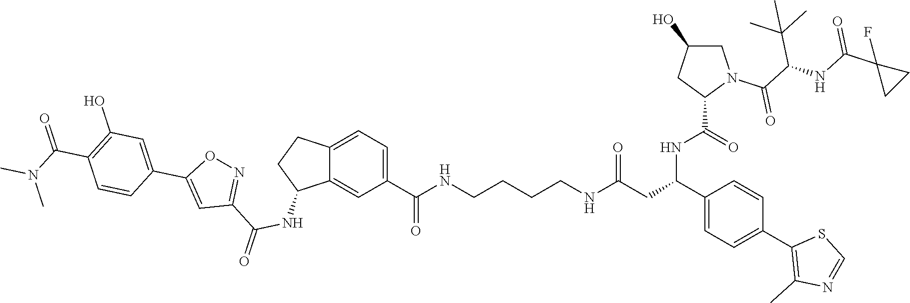 [SMILES: Cc1ncsc1-c1ccc([C@H](CC(=O)NCCCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-((4-((S)- 3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5-yl) phenyl)propanamido)butyl)carba- moyl)-2,3-dihydro-1H-inden-1- yl)isoxazole-3-carboxamide |
| 191 | LQ108- 68 | 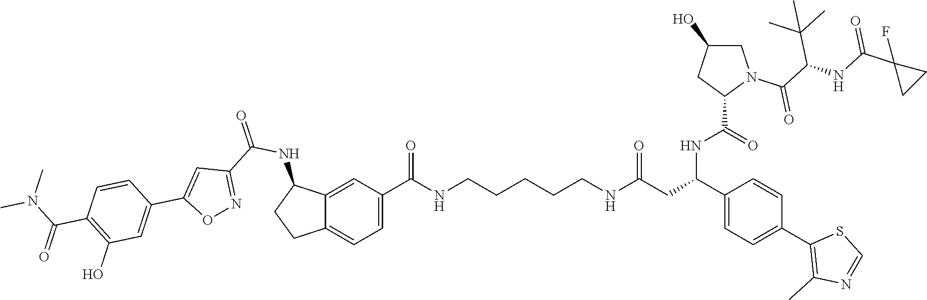 [SMILES: Cc1ncsc1-c1ccc([C@H](CC(=O)NCCCCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-((5-((S)- 3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5-yl) phenyl)propanamido)pentyl)carba- moyl)-2,3-dihydro-1H-inden-1- yl)isoxazole-3-carboxamide |
| 192 | LQ108- 69 | 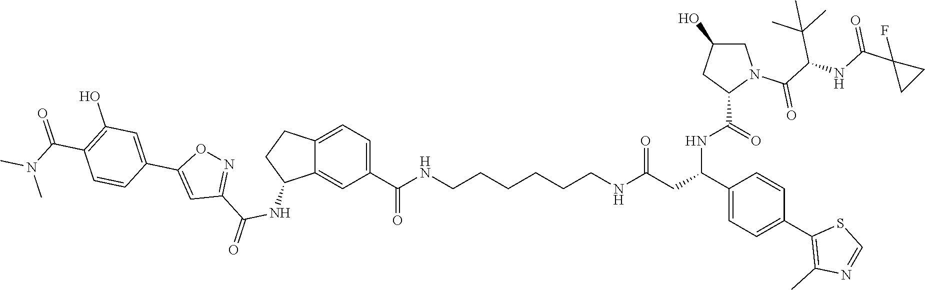 [SMILES: Cc1ncsc1-c1ccc([C@H](CC(=O)NCCCCCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-((6-((S)- 3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5-yl) phenyl)propanamido)hexyl)carba- moyl)-2,3-dihydro-1H-inden-1- yl)isoxazole-3-carboxamide |
| 193 | LLQ108- 70 | 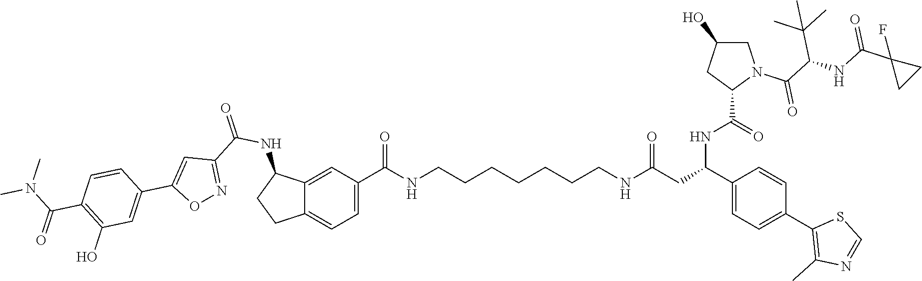 [SMILES: Cc1ncsc1-c1ccc([C@H](CC(=O)NCCCCCCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-((7-((S)- 3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5-yl) phenyl)propanamido)heptyl)carba- moyl)-2,3-dihydro-1H-inden-1- yl)isoxazole-3-carboxamide |
| 194 | LQ108- 71 | 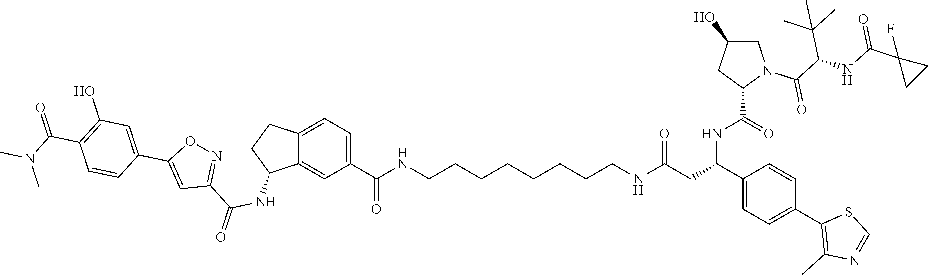 [SMILES: Cc1ncsc1-c1ccc([C@H](CC(=O)NCCCCCCCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-((8-((S)- 3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5-yl) phenyl)propanamido)octyl)carba- moyl)-2,3-dihydro-1H-inden-1- yl)isoxazole-3-carboxamide |
| 195 | LQ108- 72 | 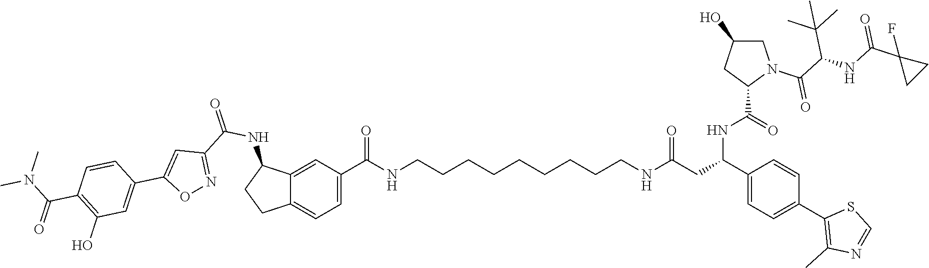 [SMILES: Cc1ncsc1-c1ccc([C@H](CC(=O)NCCCCCCCCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-((9-((S)- 3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5-yl) phenyl)propanamido)nonyl)carba- moyl)-2,3-dihydro-1H-inden-1- yl)isoxazole-3-carboxamide |
| 196 | LQ108- 73 | 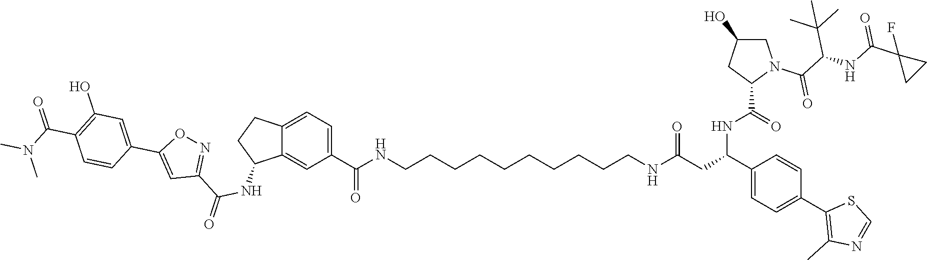 [SMILES: Cc1ncsc1-c1ccc([C@H](CC(=O)NCCCCCCCCCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-((10- ((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5-yl) phenyl)propanamido)decyl)carba- moyl)-2,3-dihydro-1H-inden-1- yl)isoxazole-3-carboxamide |
| 197 | LQ108- 74 | 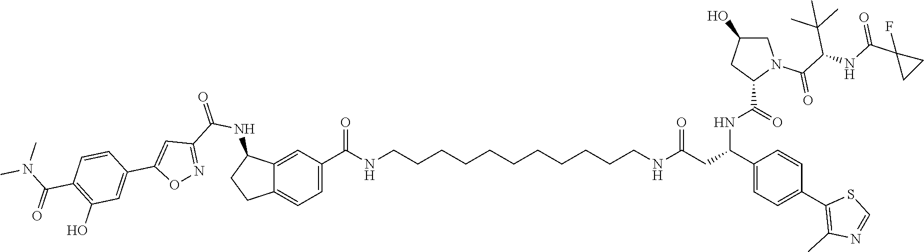 [SMILES: Cc1ncsc1-c1ccc([C@H](CC(=O)NCCCCCCCCCCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-((11-((S)- 3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5-yl) phenyl)propanamido)undecyl)car- bamoyl)-2,3-dihydro-1H-inden-1- yl)isoxazole-3-carboxamide |
| 198 | LQ108- 75 | 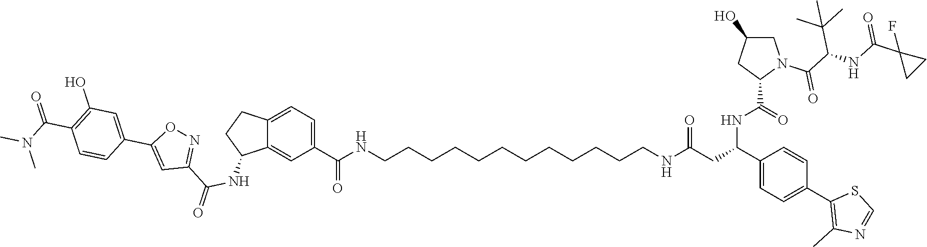 [SMILES: Cc1ncsc1-c1ccc([C@H](CC(=O)NCCCCCCCCCCCCNC(=O)c2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-((12-((S)- 3-((2S,4R)-1-((S)-2-(1- dfluorocyclopropane-1- carboxamido)-3,3- dimethylbutanoyl)-4- hydroxypyrrolidine-2- carboxamido)-3-(4-(4- methylthiazol-5-yl) phenyl)propanamido)dodecyl)car- bamoyl)-2,3-dihydro-1H-inden-1- yl)isoxazole-3-carboxamide |
| 199 | LQ126- 46 | 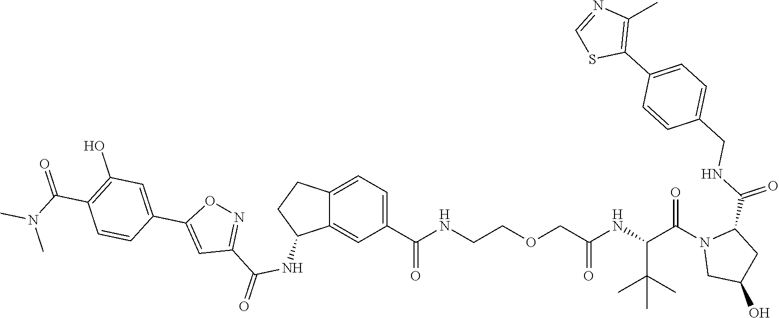 [SMILES: Cc1ncsc1-c1ccc(CNC(=O)[C@@H]2C[C@@H](O)CN2C(=O)[C@@H](NC(=O)COCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-((2-(2- (((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-2- oxoethoxy)ethyl)carbamoyl)-2,3- dihydro-1H-inden-1-yl)isoxazole-3- carboxamide |
| 200 | LQ126- 47 | 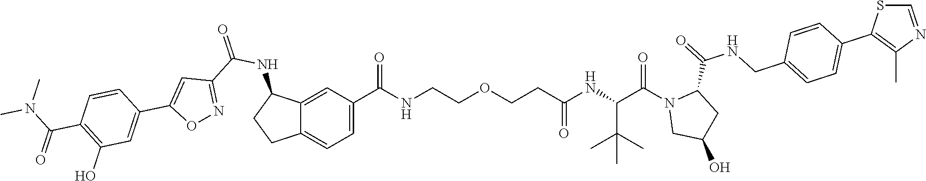 [SMILES: Cc1ncsc1-c1ccc(CNC(=O)[C@@H]2C[C@@H](O)CN2C(=O)[C@@H](NC(=O)CCOCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-((2-(3- (((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-3- oxopropoxy)ethyl)carbamoyl)-2,3- dihydro-1H-inden-1-yl)isoxazole-3- carboxamide |
| 201 | LQ126- 49 | 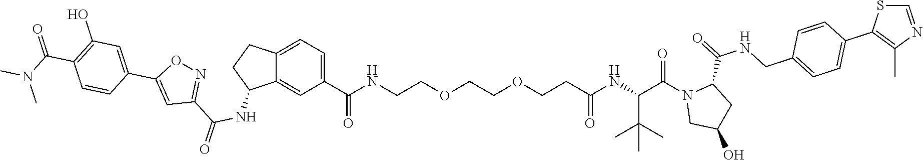 [SMILES: Cc1ncsc1-c1ccc(CNC(=O)[C@@H]2C[C@@H](O)CN2C(=O)[C@@H](NC(=O)CCOCCOCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-((2-(2-(3- (((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-3-oxo- propoxy)ethoxy)ethyl)carbamoyl)- 2,3-dihydro-1H-inden-1- yl)isoxazole-3-carboxamide |
| 202 | LQ126- 50 | 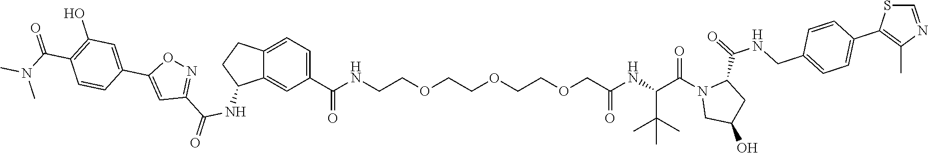 [SMILES: Cc1ncsc1-c1ccc(CNC(=O)[C@@H]2C[C@@H](O)CN2C(=O)[C@@H](NC(=O)COCCOCCOCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(((S)-13- ((2S,4R)-4-hydroxy-2-((4-(4- methylthiazol-5- yl)benzyl)carbamoyl)pyrrolidine-1- carbonyl)-14,14-dimethyl-11-oxo- 3,6,9-trioxa-12- azapentadecyl)carbamoyl)-2,3- dihydro-1H-inden-1-yl)isoxazole-3- carboxamide |
| 203 | LQ126- 51 | 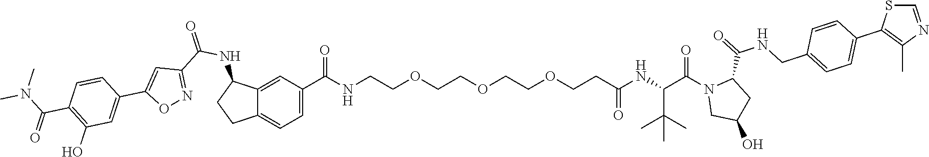 [SMILES: Cc1ncsc1-c1ccc(CNC(=O)[C@@H]2C[C@@H](O)CN2C(=O)[C@@H](NC(=O)CCOCCOCCOCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(((S)-14- ((2S,4R)-4-hydroxy-2-((4-(4- methylthiazol-5- yl)benzyl)carbamoyl)pyrrolidine-1- carbonyl)-15,15-dimethyl-12-oxo- 3,6,9-trioxa-13- azahexadecyl)carbamoyl)-2,3- dihydro-1H-inden-1-yl)isoxazole-3- carboxamide |
| 204 | LQ126- 52 | 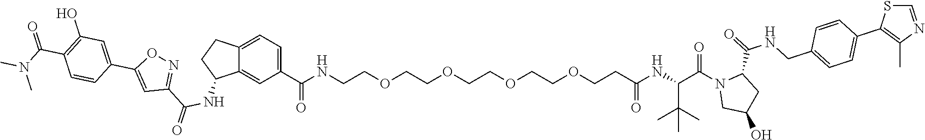 [SMILES: Cc1ncsc1-c1ccc(CNC(=O)[C@@H]2C[C@@H](O)CN2C(=O)[C@@H](NC(=O)CCOCCOCCOCCOCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(((S)-17- ((2S,4R)-4-hydroxy-2-((4-(4- methylthiazol-5- yl)benzyl)carbamoyl)pyrrolidine-1- carbonyl)-18,18-dimethyl-15-oxo- 3,6,9,12-tetraoxa-16- azanonadecyl)carbamoyl)-2,3- dihydro-1H-inden-1-yl)isoxazole-3- |
| | | | carboxamide |
| 205 | LQ126- 53 | 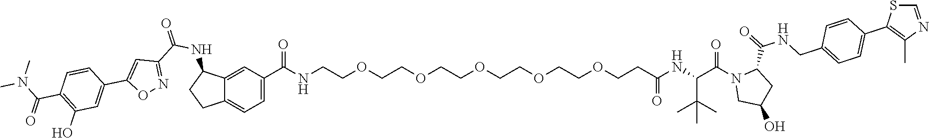 [SMILES: Cc1ncsc1-c1ccc(CNC(=O)[C@@H]2C[C@@H](O)CN2C(=O)[C@@H](NC(=O)CCOCCOCCOCCOCCOCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(((S)-20- ((2S,4R)-4-hydroxy-2-((4-(4- methylthiazol-5- yl)benzyl)carbamoyl)pyrrolidine-1- carbonyl)-21,21-dimethyl-18-oxo- 3,6,9,12,15-pentaoxa-19-aza- docosyl)carbamoyl)-2,3-dihydro- 1H-inden-1-yl)isoxazole-3- |
| | | | carboxamide |
| 206 | LQ126- 54 | 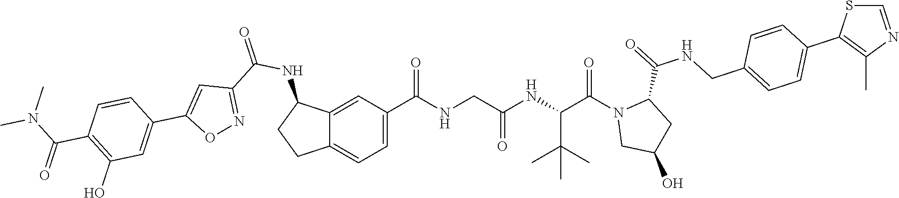 [SMILES: Cc1ncsc1-c1ccc(CNC(=O)[C@@H]2C[C@@H](O)CN2C(=O)[C@@H](NC(=O)CNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-((2-(((S)- 1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-2-oxoethyl)carbamoyl)- 2,3-dihydro-1H-inden-1- yl)isoxazole-3-carboxamide |
| 207 | LQ126- 55 | 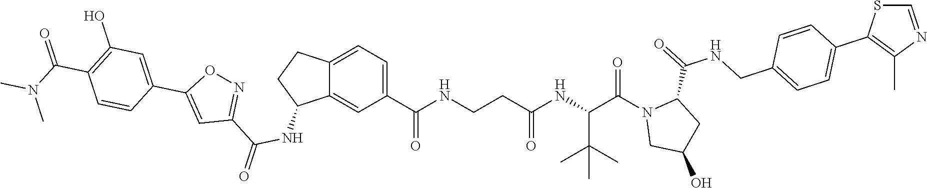 [SMILES: Cc1ncsc1-c1ccc(CNC(=O)[C@@H]2C[C@@H](O)CN2C(=O)[C@@H](NC(=O)CCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-((3-(((S)- 1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-3-oxopropyl)carbamoyl)- 2,3-dihydro-1H-inden-1- yl)isoxazole-3-carboxamide |
| 208 | LQ126- 56 | 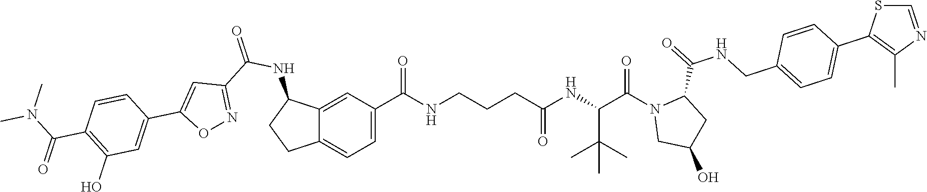 [SMILES: Cc1ncsc1-c1ccc(CNC(=O)[C@@H]2C[C@@H](O)CN2C(=O)[C@@H](NC(=O)CCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-((4-(((S)- 1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-4-oxobutyl)carbamoyl)- 2,3-dihydro-1H-inden-1- yl)isoxazole-3-carboxamide |
| 209 | LQ126- 57 | 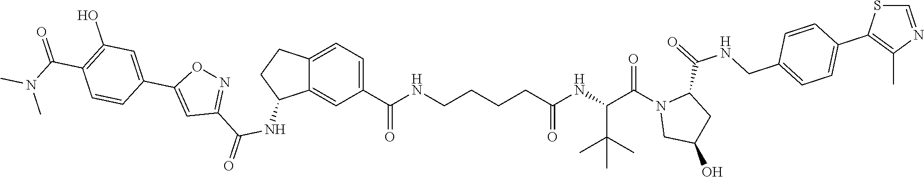 [SMILES: Cc1ncsc1-c1ccc(CNC(=O)[C@@H]2C[C@@H](O)CN2C(=O)[C@@H](NC(=O)CCCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-((5-(((S)- 1-((2S,4R)-4-hydroxy-2-((4-(4- methylthiazol-5- y)benzyl)carbamoyl)pyrrolidin-1- yl)-3,3-dimethyl-1-oxobutan-2- yl)amino)-5-oxopentyl)carbamoyl)- 2,3-dihydro-1H-inden-1- yl)isoxazole-3-carboxamide |
| 210 | LQ126- 58 | 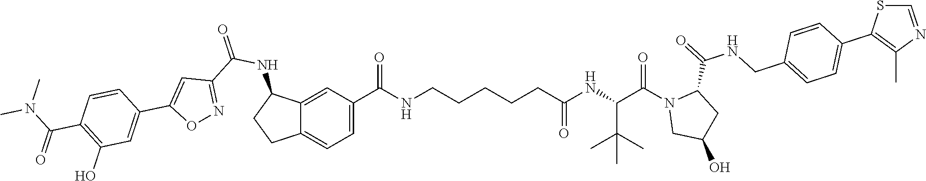 [SMILES: Cc1ncsc1-c1ccc(CNC(=O)[C@@H]2C[C@@H](O)CN2C(=O)[C@@H](NC(=O)CCCCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-((6-(((S)- 1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-6-oxohexyl)carbamoyl)- 2,3-dihydro-1H-inden-1- yl)isoxazole-3-carboxamide |
| 211 | LQ126- 59 | 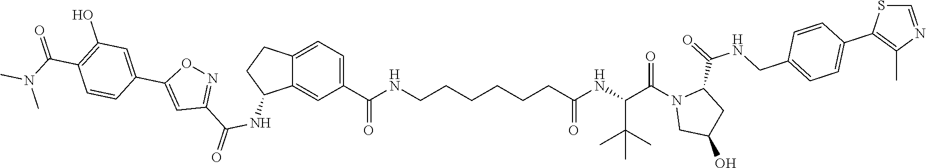 [SMILES: Cc1ncsc1-c1ccc(CNC(=O)[C@@H]2C[C@@H](O)CN2C(=O)[C@@H](NC(=O)CCCCCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-((7-(((S)- 1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-7-oxoheptyl)carbamoyl)- 2,3-dihydro-1H-inden-1- yl)isoxazole-3-carboxamide |
| 212 | LQ126- 60 | 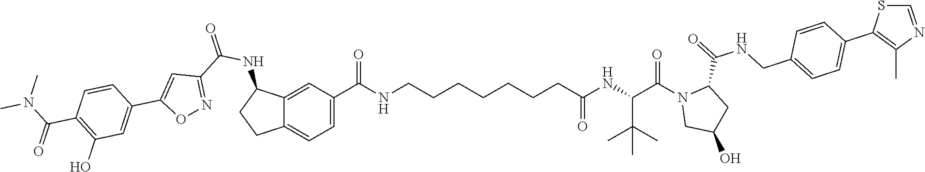 [SMILES: Cc1ncsc1-c1ccc(CNC(=O)[C@@H]2C[C@@H](O)CN2C(=O)[C@@H](NC(=O)CCCCCCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-((8-(((S)- 1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-8-oxooctyl)carbamoyl)- 2,3-dihydro-1H-inden-1- yl)isoxazole-3-carboxamide |
| 213 | LQ126- 61 | 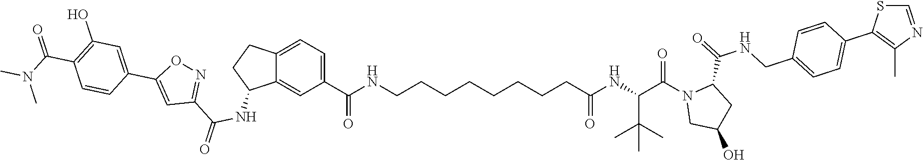 [SMILES: Cc1ncsc1-c1ccc(CNC(=O)[C@@H]2C[C@@H](O)CN2C(=O)[C@@H](NC(=O)CCCCCCCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-((9-(((S)- 1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-9-oxononyl)carbamoyl)- 2,3-dihydro-1H-inden-1- yl)isoxazole-3-carboxamide |
| 214 | LQ126- 62 | 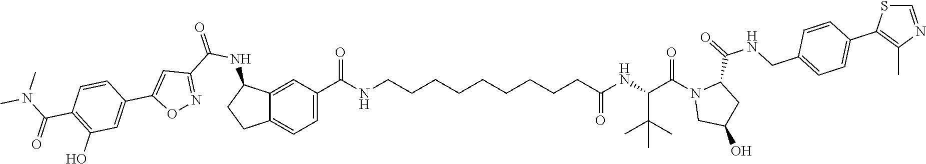 [SMILES: Cc1ncsc1-c1ccc(CNC(=O)[C@@H]2C[C@@H](O)CN2C(=O)[C@@H](NC(=O)CCCCCCCCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3-hy- droxyphenyl)-N-((R)-6-((10-(((S)- 1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-10-oxodecyl)carbamoyl)- 2,3-dihydro-1H-inden-1- yl)isoxazole-3-carboxamide |
| 215 | LQ126- 63 | 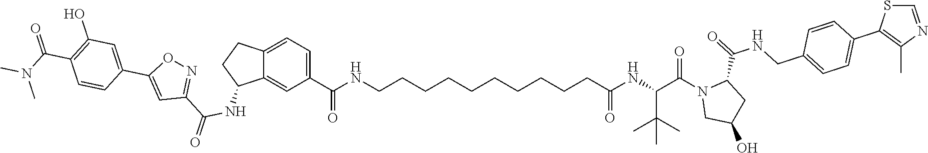 [SMILES: Cc1ncsc1-c1ccc(CNC(=O)[C@@H]2C[C@@H](O)CN2C(=O)[C@@H](NC(=O)CCCCCCCCCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3-hy- droxyphenyl)-N-((R)-6-((11-(((S)- 1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-11- oxoundecyl)carbamoyl)-2,3- dihydro-1H-inden-1-yl)isoxazole-3- carboxamide |
| 216 | LQ126- 77 | 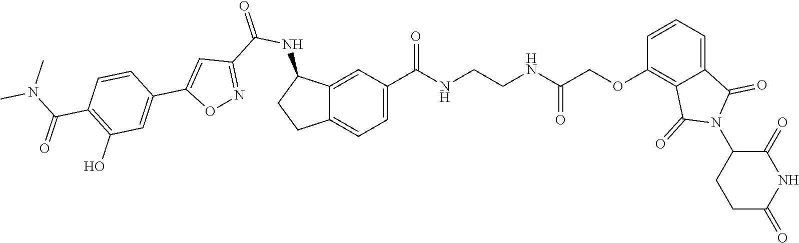 [SMILES: CN(C)C(=O)c1ccc(-c2cc(C(=O)N[C@@H]3CCc4ccc(C(=O)NCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-((2-(2- ((2-(2,6-dioxopiperidin-3-yl)-1,3- dioxoisoindolin-4- yl)oxy)acetamido)ethyl)carbamoyl)- 2,3-dihydro-1H-inden-1- yl)isoxazole-3-carboxamide |
| 217 | LQ126- 78 | 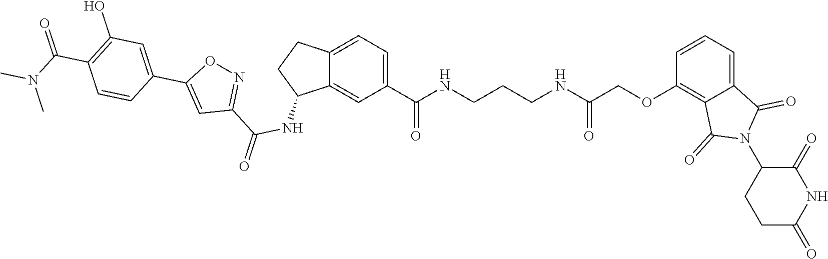 [SMILES: CN(C)C(=O)c1ccc(-c2cc(C(=O)N[C@@H]3CCc4ccc(C(=O)NCCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-((3-(2- ((2-(2,6-dioxopiperidin-3-yl)-1,3- dioxoisoindolin-4-yl) oxy)acetamido)propyl)carbamoyl)- 2,3-dihydro-1H-inden-1- yl)isoxazole-3-carboxamide |
| 218 | LQ126- 79 | 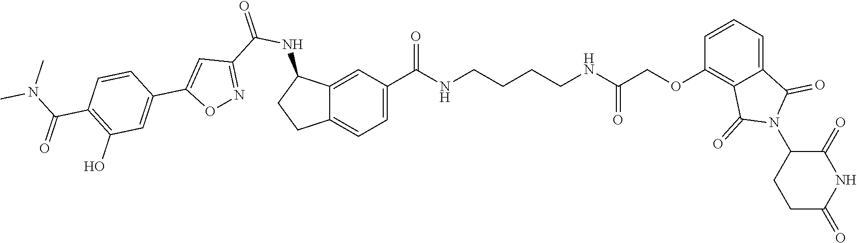 [SMILES: CN(C)C(=O)c1ccc(-c2cc(C(=O)N[C@@H]3CCc4ccc(C(=O)NCCCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-((4-(2- ((2-(2,6-dioxopiperidin-3-yl)-1,3- dioxoisoindolin-4-yl) oxy)acetamido)butyl)carbamoyl)- 2,3-dihydro-1H-inden-1- yl)isoxazole-3-carboxamide |
| 219 | LQ126- 80 | 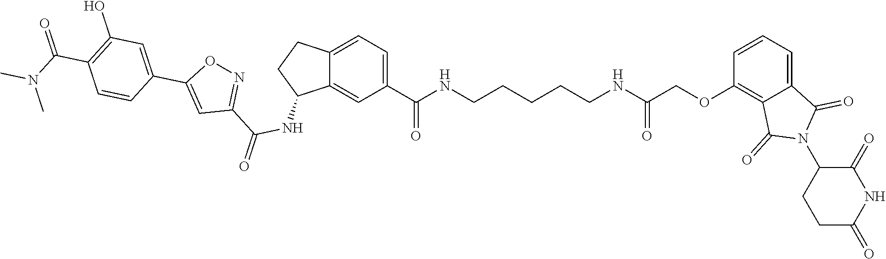 [SMILES: CN(C)C(=O)c1ccc(-c2cc(C(=O)N[C@@H]3CCc4ccc(C(=O)NCCCCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-((5-(2- ((2-(2,6-dioxopiperidin-3-yl)-1,3- dioxoisoindolin-4-yl) oxy)acetamido)pentyl)carbamoyl)- 2,3-dihydro-1H-inden-1- yl)isoxazole-3-carboxamide |
| 220 | LQ126- 81 | 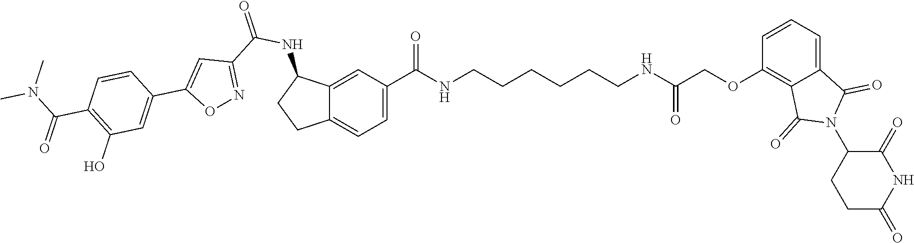 [SMILES: CN(C)C(=O)c1ccc(-c2cc(C(=O)N[C@@H]3CCc4ccc(C(=O)NCCCCCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-((6-(2- ((2-(2,6-dioxopiperidin-3-yl)-1,3- dioxoisoindolin-4-yl) oxy)acetamido)hexyl)carbamoyl)- 2,3-dihydro-1H-inden-1- yl)isoxazole-3-carboxamide |
| 221 | LQ126- 82 | 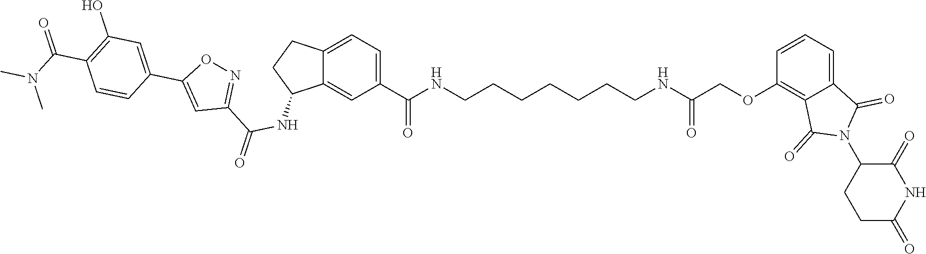 [SMILES: CN(C)C(=O)c1ccc(-c2cc(C(=O)N[C@@H]3CCc4ccc(C(=O)NCCCCCCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-((7-(2- ((2-(2,6-dioxopiperidin-3-yl)-1,3- dioxoisoindolin-4-yl) oxy)acetamido)heptyl)carbamoyl)- 2,3-dihydro-1H-inden-1- yl)isoxazole-3-carboxamide |
| 222 | LQ126- 83 | 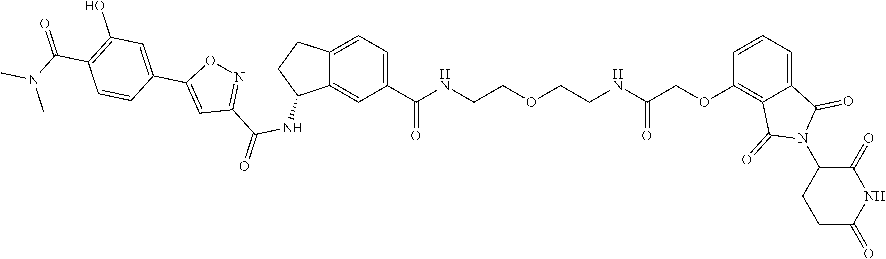 [SMILES: CN(C)C(=O)c1ccc(-c2cc(C(=O)N[C@@H]3CCc4ccc(C(=O)NCCOCCNC(=O)COc5cccc6c5C(=O)N(C5CCC(=O)NC5=O)C6=O)cc43)no2)cc1O] | 5-(4-(dimethylcarbamoyl)-3-hy- droxyphenyl)-N-((1R)-6-((2-(2-(2- ((2-(2,6-dioxopiperidin-3-yl)-1,3- dioxoisoindolin-4-yl) oxy)acetamido)ethoxy)ethyl)carba- moyl)-2,3-dihydro-1H-inden-1- yl)isoxazole-3-carboxamide |
| 223 | LQ126- 84 | 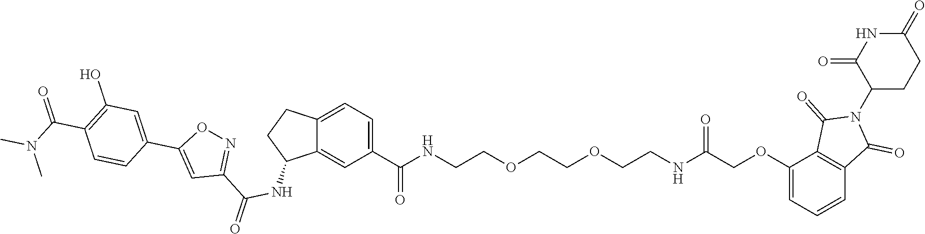 [SMILES: CN(C)C(=O)c1ccc(-c2cc(C(=O)N[C@@H]3CCc4ccc(C(=O)NCCOCCOCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-((2- (2-(2-(2-((2-(2,6-dioxopiperidin- 3-yl)-1,3-dioxoisoindolin-4-yl) oxy)acetamido)ethoxy)ethoxy)eth- yl)carbamoyl)2,3-dihydro-1H- inden-1-yl)isoxazole-3- carboxamide |
| 224 | LQ126- 85 | 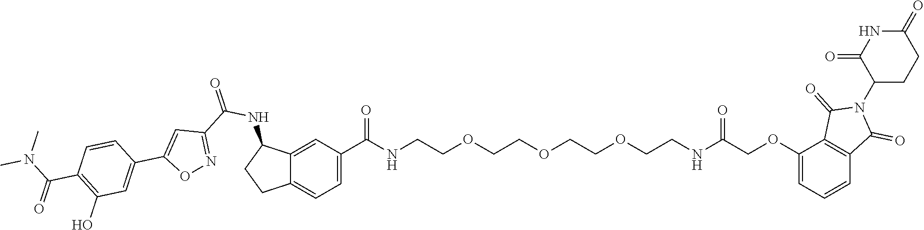 [SMILES: CN(C)C(=O)c1ccc(-c2cc(C(=O)N[C@@H]3CCc4ccc(C(=O)NCCOCCOCCOCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-((1-((2- (2,6-dioxopiperidin-3-yl)-1,3- dioxoisoindolin-4-yl)oxy)-2-oxo- 6,9,12-trioxa-3-azatetradecan-14- yl)carbamoyl)-2,3-dihydro-1H- inden-1-yl)isoxazole-3- carboxamide |
| 225 | LQ126- 86 | 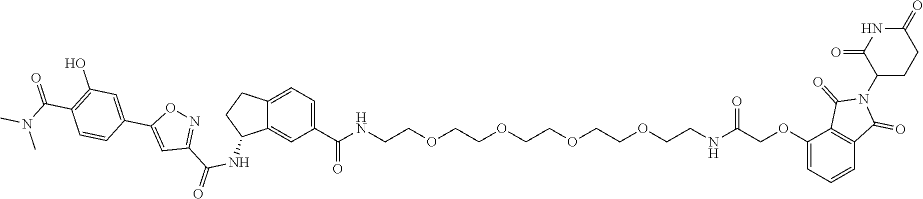 [SMILES: CN(C)C(=O)c1ccc(-c2cc(C(=O)N[C@@H]3CCc4ccc(C(=O)NCCOCCOCCOCCOCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-((1-((2- (2,6-dioxopiperidin-3-yl)-1,3- dioxoisoindolin-4-yl)oxy)-2-oxo- 6,9,12,15-tetraoxa-3-azaheptadecan- 17-yl)carbamoyl)-2,3-dihydro-1H- inden-1-yl)isoxazole-3- carboxamide |
| 226 | LQ126- 87 | 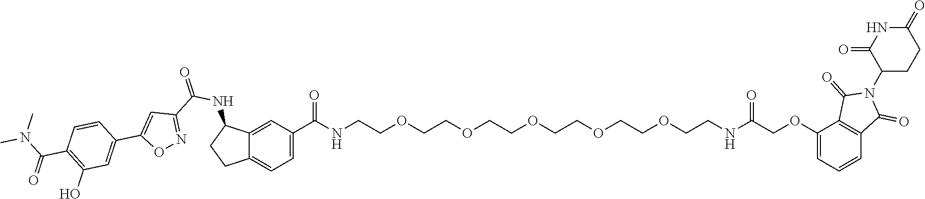 [SMILES: CN(C)C(=O)c1ccc(-c2cc(C(=O)N[C@@H]3CCc4ccc(C(=O)NCCOCCOCCOCCOCCOCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-((1-((2- (2,6-dioxopiperidin-3-yl)-1,3- dioxoisoindolin-4-yl)oxy)-2-oxo- 6,9,12,15,18-pentaoxa-3-azaicosan- 20-yl)carbamoyl)-2,3-dihydro-1H- inden-1-yl)isoxazole-3- carboxamide |
| 228 | LQ126- 89 | 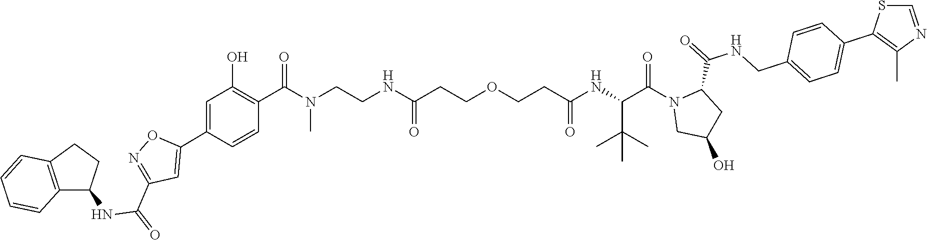 [SMILES: Cc1ncsc1-c1ccc(CNC(=O)[C@@H]2C[C@@H](O)CN2C(=O)[C@@H](NC(=O)CCOCCC(=O)NCCN(C)C(=O)c2ccc(-c3cc(C(=O)N[C@@H]4CCc5ccccc54)no3)cc2O)C(C)(C)C)cc1] | N-((R)-2,3-dihydro-1H-inden-1-yl)- 5-(3-hydroxy-4-((2-(3-(3-(((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)amino)-3-oxo- propoxy)propanamido)ethyl)meth- yl)carbamoyl)phenyl)isoxazole-3- carboxamide |
| 229 | LQ126- 90 | 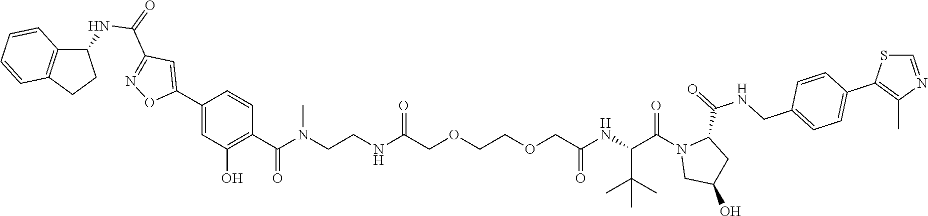 [SMILES: Cc1ncsc1-c1ccc(CNC(=O)[C@@H]2C[C@@H](O)CN2C(=O)[C@@H](NC(=O)COCCOCC(=O)NCCN(C)C(=O)c2ccc(-c3cc(C(=O)N[C@@H]4CCc5ccccc54)no3)cc2O)C(C)(C)C)cc1] | N-((R)-2,3-dihydro-1H-inden-1-yl)- 5-(3-hydroxy-4-(((S)-13-((2S,4R)- 4-hydroxy-2-((4-(4-methylthiazol- 5-yl)benzyl)carbamoyl)pyrrolidine- 1-carbonyl)-14,14-dimethyl- 4,11-dioxo-6,9-dioxa-3,12-di- azapentadecyl)(methyl)carbamoyl) phenyl)isoxazole-3-carboxamide |
| 230 | LQ126- 91 | 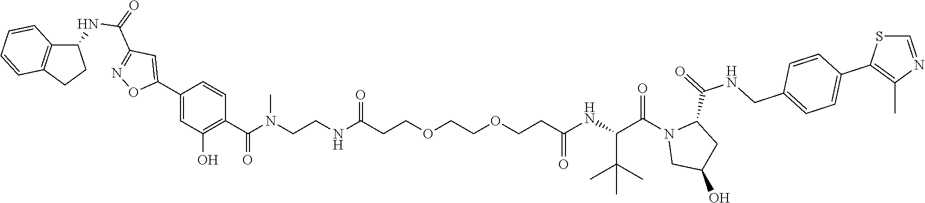 [SMILES: Cc1ncsc1-c1ccc(CNC(=O)[C@@H]2C[C@@H](O)CN2C(=O)[C@@H](NC(=O)CCOCCOCCC(=O)NCCN(C)C(=O)c2ccc(-c3cc(C(=O)N[C@@H]4CCc5ccccc54)no3)cc2O)C(C)(C)C)cc1] | N-((R)-2,3-dihydro-1H-inden-1-yl)- 5-(3-hydroxy-4-(((S)-15-((2S,4R)- 4-hydroxy-2-((4-(4-methylthiazol- 5-yl)benzyl)carbamoyl)pyrrolidine- 1-carbonyl)-16,16-dimethyl-4,13- dioxo-7,10-dioxa-3,14-di- azaheptadecyl)(methyl)carbamoyl) phenyl)isoxazole-3-carboxamide |
| 231 | LQ126- 92 | 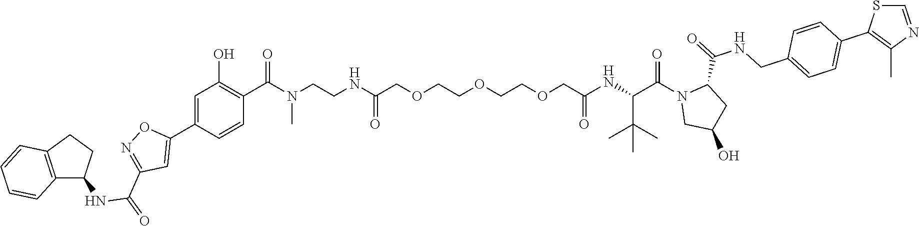 [SMILES: Cc1ncsc1-c1ccc(CNC(=O)[C@@H]2C[C@@H](O)CN2C(=O)[C@@H](NC(=O)COCCOCCOCC(=O)NCCN(C)C(=O)c2ccc(-c3cc(C(=O)N[C@@H]4CCc5ccccc54)no3)cc2O)C(C)(C)C)cc1] | N-((R)-2,3-dihydro-1H-inden-1-yl)- 5-(3-hydroxy-4-(((S)-16-((2S,4R)- 4-hydroxy-2-((4-(4-methylthiazol- 5-yl)benzyl)carbamoyl)pyrrolidine- 1-carbonyl)-17,17-dimethyl-4,14- dioxo-6,9,12-trioxa-3,15- diazaoctadecyl)(methyl)carbamoyl) phenyl)isoxazole-3-carboxamide |
| 232 | LQ126- 93 | 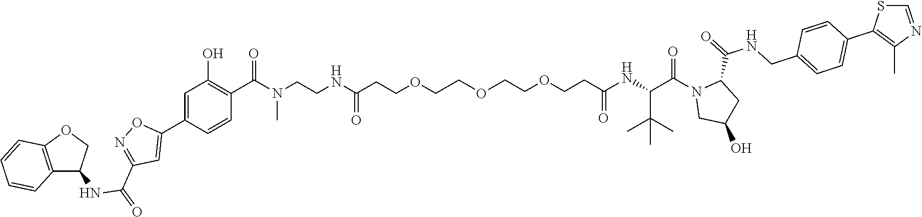 [SMILES: Cc1ncsc1-c1ccc(CNC(=O)[C@@H]2C[C@@H](O)CN2C(=O)[C@@H](NC(=O)CCOCCOCCOCCC(=O)NCCN(C)C(=O)c2ccc(-c3cc(C(=O)N[C@@H]4COc5ccccc54)no3)cc2O)C(C)(C)C)cc1] | N-((R)-2,3-dihydro-1H-inden-1-yl)- 5-(3-hydroxy-4-(((S)-18-((2S,4R)- 4-hydroxy-2-((4-(4-methylthiazol- 5-yl)benzyl)carbamoyl)pyrrolidine- 1-carbonyl)-19,19-dimethyl-4,16- dioxo-7,10,13-trioxa-3,17-di- azaicosyl)(methyl)carbamoyl)phe- nyl)isoxazole-3-carboxamide |
| 233 | LQ126- 94 | 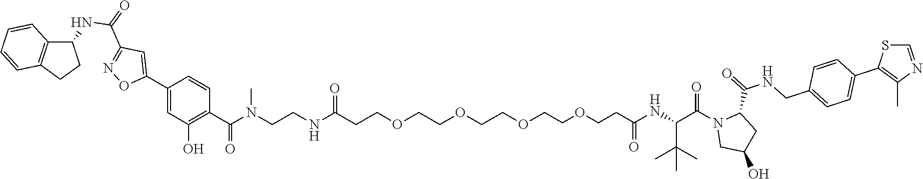 [SMILES: Cc1ncsc1-c1ccc(CNC(=O)[C@@H]2C[C@@H](O)CN2C(=O)[C@@H](NC(=O)CCOCCOCCOCCOCCC(=O)NCCN(C)C(=O)c2ccc(-c3cc(C(=O)N[C@@H]4CCc5ccccc54)no3)cc2O)C(C)(C)C)cc1] | N¹-(2-(4-(3-(((R)-2,3-dihydro-1H- inden-1-yl)carbamoyl)isoxazol-5- yl)-2-hydroxy-N- methylbenzamido)ethyl)-N¹⁶-((S)-1- ((2S,4R)-4-hydroxy-2-((4-(4- methylthiazol-5- yl)benzyl)carbamoyl)pyrrolidin-1- yl)-3,3-dimethyl-1-oxobutan-2-yl)- 4,7,10,13- tetraoxahexadecanediamide |
| 234 | LQ126- 95 | 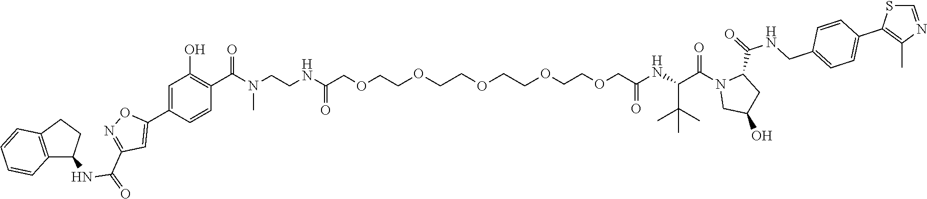 [SMILES: Cc1ncsc1-c1ccc(CNC(=O)[C@@H]2C[C@@H](O)CN2C(=O)[C@@H](NC(=O)COCCOCCOCCOCCOCC(=O)NCCN(C)C(=O)c2ccc(-c3cc(C(=O)N[C@@H]4CCc5ccccc54)no3)cc2O)C(C)(C)C)cc1] | N¹-(2-(4-(3-(((R)-2,3-dihydro-1H- inden-1-yl)carbamoyl)isoxazol-5- yl)-2-hydroxy-N- methylbenzamido)ethyl)-N¹⁷-((S)-1- ((2S,4R)-4-hydroxy-2-((4-(4- methylthiazol-5- yl)benzyl)carbamoyl)pyrrolidin-1- yl)-3,3-dimethyl-1-oxobutan-2-yl)- 3,6,9,12,15- pentaoxaheptadecanediamide |
| 235 | Q126- 96 | 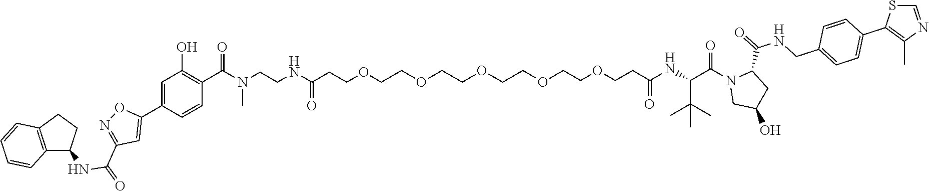 [SMILES: Cc1ncsc1-c1ccc(CNC(=O)[C@@H]2C[C@@H](O)CN2C(=O)[C@@H](NC(=O)CCOCCOCCOCCOCCOCCC(=O)NCCN(C)C(=O)c2ccc(-c3cc(C(=O)N[C@@H]4CCc5ccccc54)no3)cc2O)C(C)(C)C)cc1] | N¹-(2-(4-(3-(((R)-2,3-dihydro-1H- inden-1-yl)carbamoyl)isoxazol-5- yl)-2-hydroxy-N- methylbenzamido)ethyl)-N¹⁹-((S)-1- ((2S,4R)-4-hydroxy-2-((4-(4- methylthiazol-5- yl)benzyl)carbamoyl)pyrrolidin-1- yl)-3,3-dimethyl-1-oxobutan-2-yl)- 4,7,10,13,16- pentaoxanonadecanediamide |
| 236 | LQ126- 97 | 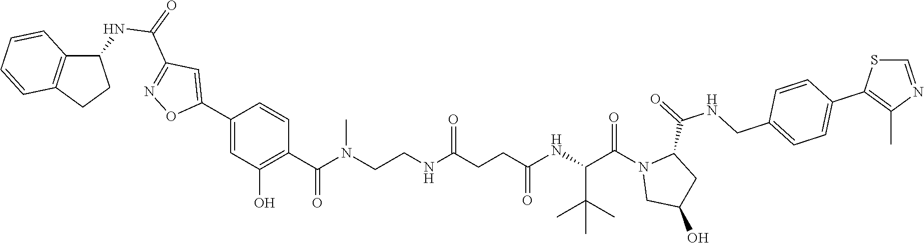 [SMILES: Cc1ncsc1-c1ccc(CNC(=O)[C@@H]2C[C@@H](O)CN2C(=O)[C@@H](NC(=O)CCC(=O)NCCN(C)C(=O)c2ccc(-c3cc(C(=O)N[C@@H]4CCc5ccccc54)no3)cc2O)C(C)(C)C)cc1] | N¹-(2-(4-(3-(((R)-2,3-dihydro-1H- inden-1-yl)carbamoyl)isoxazol-5- yl)-2-hydroxy-N- methylbenzamido)ethyl)-N⁴-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)succinamide |
| 237 | LQ126- 98 | 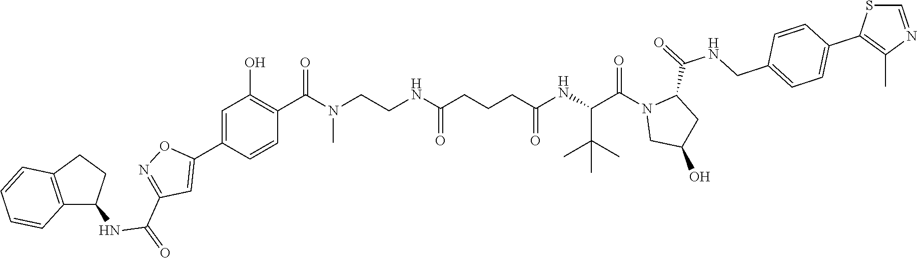 [SMILES: Cc1ncsc1-c1ccc(CNC(=O)[C@@H]2C[C@@H](O)CN2C(=O)[C@@H](NC(=O)CCCC(=O)NCCN(C)C(=O)c2ccc(-c3cc(C(=O)N[C@@H]4CCc5ccccc54)no3)cc2O)C(C)(C)C)cc1] | N¹-(2-(4-(3-(((R)-2,3-dihydro-1H- inden-1-yl)carbamoyl)isoxazol-5- yl)-2-hydroxy-N- methylbenzamido)ethyl)-N⁵-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)glutaramide |
| 238 | LQ126- 99 | 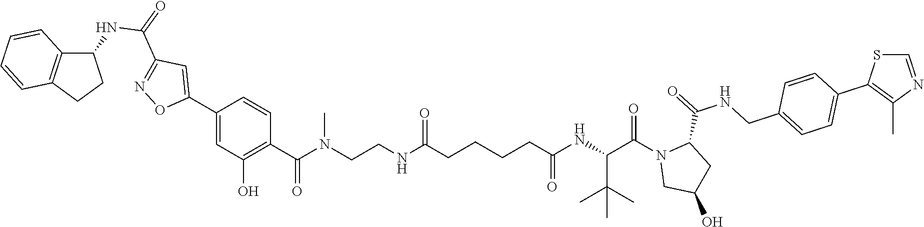 [SMILES: Cc1ncsc1-c1ccc(CNC(=O)[C@@H]2C[C@@H](O)CN2C(=O)[C@@H](NC(=O)CCCCC(=O)NCCN(C)C(=O)c2ccc(-c3cc(C(=O)N[C@@H]4CCc5ccccc54)no3)cc2O)C(C)(C)C)cc1] | N¹-(2-(4-(3-(((R)-2,3-dihydro-1H- inden-1-yl)carbamoyl)isoxazol-5- yl)-2-hydroxy-N- methylbenzamido)ethyl)-N⁶-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)adipamide |
| 239 | LQ126-100 | 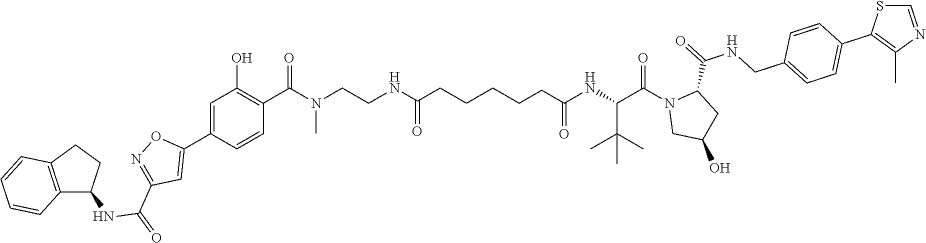 [SMILES: Cc1ncsc1-c1ccc(CNC(=O)[C@@H]2C[C@@H](O)CN2C(=O)[C@@H](NC(=O)CCCCCC(=O)NCCN(C)C(=O)c2ccc(-c3cc(C(=O)N[C@@H]4CCc5ccccc54)no3)cc2O)C(C)(C)C)cc1] | N¹-(2-(4-(3-(((R)-2,3-dihydro-1H- inden-1-yl)carbamoyl)isoxazol-5- yl)-2-hydroxy-N- methylbenzamido)ethyl)-N⁷-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)heptanediamide |
| 240 | LQ126- 101 | 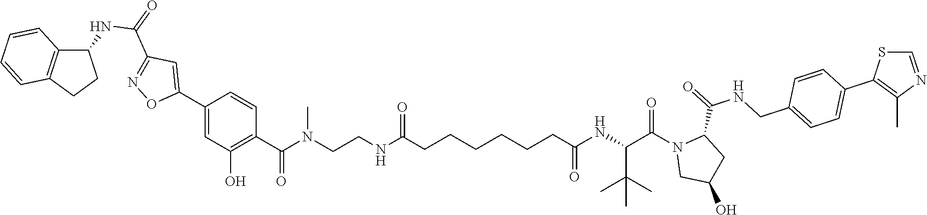 [SMILES: Cc1ncsc1-c1ccc(CNC(=O)[C@@H]2C[C@@H](O)CN2C(=O)[C@@H](NC(=O)CCCCCCC(=O)NCCN(C)C(=O)c2ccc(-c3cc(C(=O)N[C@@H]4CCc5ccccc54)no3)cc2O)C(C)(C)C)cc1] | N¹-(2-(4-(3-(((R)-2,3-dihydro-1H- inden-1-yl)carbamoyl)isoxazol-5- yl)-2-hydroxy-N- methylbenzamido)ethyl)-N⁸-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)octanediamide |
| 241 | LQ126- 102 | 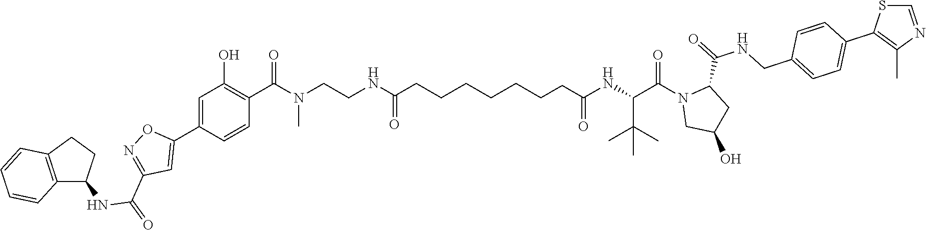 [SMILES: Cc1ncsc1-c1ccc(CNC(=O)[C@@H]2C[C@@H](O)CN2C(=O)[C@@H](NC(=O)CCCCCCCC(=O)NCCN(C)C(=O)c2ccc(-c3cc(C(=O)N[C@@H]4CCc5ccccc54)no3)cc2O)C(C)(C)C)cc1] | N¹-(2-(4-(3-(((R)-2,3-dihydro-1H- inden-1-yl)carbamoyl)isoxazol-5- yl)-2-hydroxy-N- methylbenzamido)ethyl)-N⁹-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)nonanediamide |
| 242 | LQ126- 103 | 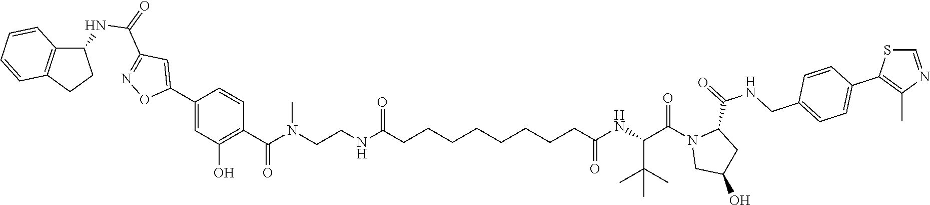 [SMILES: Cc1ncsc1-c1ccc(CNC(=O)[C@@H]2C[C@@H](O)CN2C(=O)[C@@H](NC(=O)CCCCCCCCC(=O)NCCN(C)C(=O)c2ccc(-c3cc(C(=O)N[C@@H]4CCc5ccccc54)no3)cc2O)C(C)(C)C)cc1] | N¹-(2-(4-(3-(((R)-2,3-dihydro-1H- inden-1-yl)carbamoyl)isoxazol-5- yl)-2-hydroxy-N- methylbenzamido)ethyl)-N¹⁰-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)decanediamide |
| 243 | LQ126- 104 | 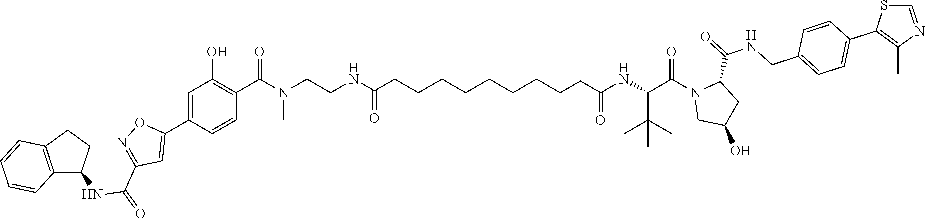 [SMILES: Cc1ncsc1-c1ccc(CNC(=O)[C@@H]2C[C@@H](O)CN2C(=O)[C@@H](NC(=O)CCCCCCCCCC(=O)NCCN(C)C(=O)c2ccc(-c3cc(C(=O)N[C@@H]4CCc5ccccc54)no3)cc2O)C(C)(C)C)cc1] | N¹-(2-(4-(3-(((R)-2,3-dihydro-1H- inden-1-yl)carbamoyl)isoxazol-5- yl)-2-hydroxy-N methylbenzamido)ethyl)-N¹¹-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)undecanediamide |
| 244 | LQ126- 105 | 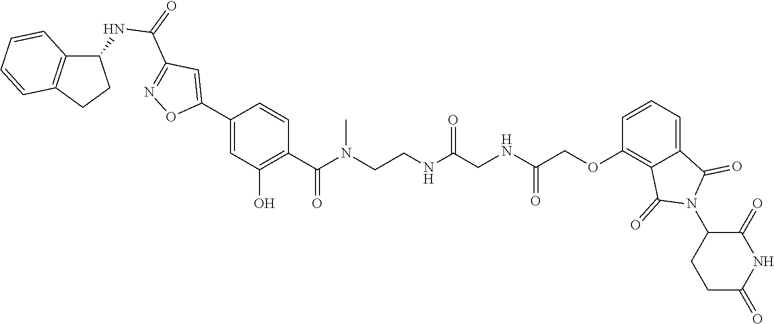 [SMILES: CN(CCNC(=O)CNC(=O)COc1cccc2c1C(=O)N(C1CCC(=O)NC1=O)C2=O)C(=O)c1ccc(-c2cc(C(=O)N[C@@H]3CCc4ccccc43)no2)cc1O] | N-((R)-2,3-dihydro-1H-inden-1-yl)- 5-(4-((2-(2-(2-((2-(2,6- dioxopiperidin-3-yl)-1,3- dioxoisoindolin-4- yl)oxy)acetamido)acetami- do)ethyl)(meth- yl)carbamoyl)-3- hydroxyphenyl)isoxazole-3- carboxamide |
| 245 | LQ126- 106 | 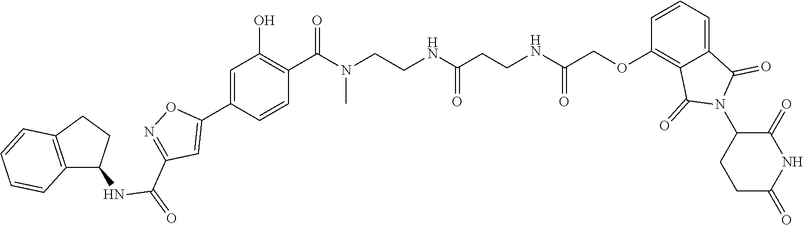 [SMILES: CN(CCNC(=O)CCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O] | N-((R)-2,3-dihydro-1H-inden-1-yl)- 5-(4-((2-(3-(2-((2-(2,6- dioxopiperidin-3-yl)-1,3- dioxoisoindolin-4- yl)oxy)acetamido)propanamido)eth- yl)(methyl)carbamoyl)-3- hydroxyphenyl)isoxazole-3- carboxamide |
| 246 | LQ126- 107 | 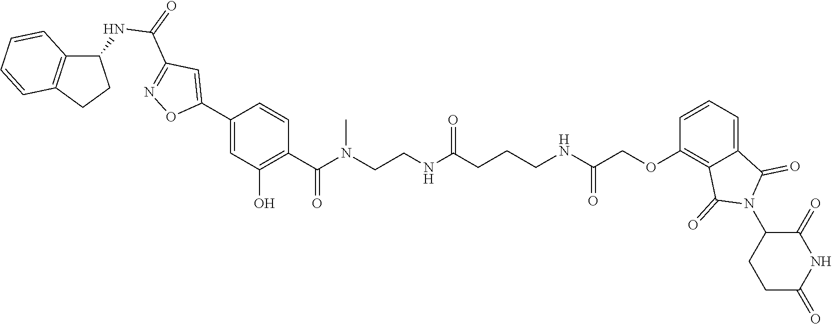 [SMILES: CN(CCNC(=O)CCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O] | N-((R)-2,3-dihydro-1H-inden-1-yl)- 5-(4-((2-(4-(2-((2-(2,6- dioxopiperidin-3-yl)-1,3- dioxoisoindolin-4-yl) oxy)acetamido)butanamido)ethyl) (methyl)carbamoyl)-3- hydroxyphenyl)isoxazole-3- carboxamide |
| 247 | LQ126- 108 | 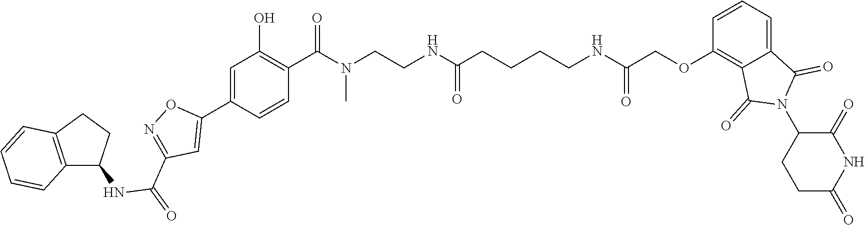 [SMILES: CN(CCNC(=O)CCCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O] | N-((R)-2,3-dihydro-1H-inden-1-yl)- 5-(4-((2-(5-(2-((2-(2,6- dioxopiperidin-3-yl)-1,3- dioxoisoindolin-4-yl) oxy)acetamido)pentanamido)ethyl) (methyl)carbamoyl)-3- hydroxyphenyl)isoxazole-3- carboxamide |
| 248 | LQ126- 109 | 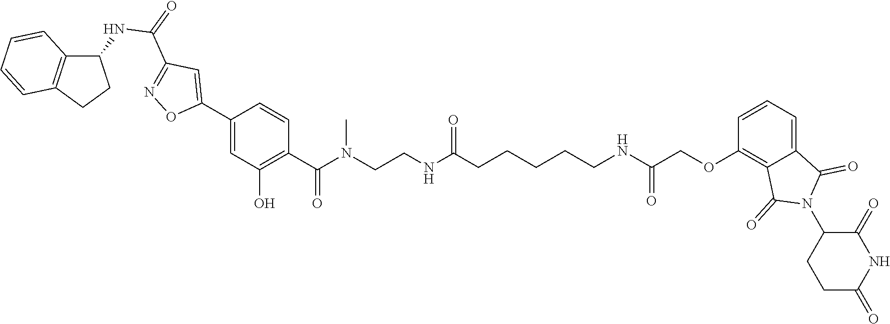 [SMILES: CN(CCNC(=O)CCCCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O] | N-((R)-2,3-dihydro-1H-inden-1-yl)- 5-(4-((2-(6-(2-((2-(2,6- dioxopiperidin-3-yl)-1,3- dioxoisoindolin-4-yl) oxy)acetamido)hexanamido)ethyl) (methyl)carbamoyl)-3- hydroxyphenyl)isoxazole-3- carboxamide |
| 249 | LQ126- 110 | 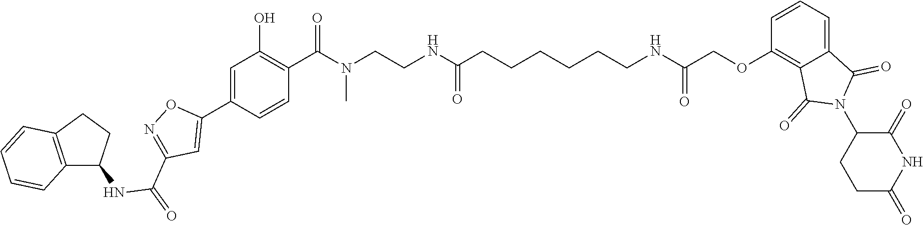 [SMILES: CN(CCNC(=O)CCCCCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O] | N-((R)-2,3-dihydro-1H-inden-1-yl)- 5-(4-((2-(7-(2-((2-(2,6- dioxopiperidin-3-yl)-1,3- dioxoisoindolin-4-yl) oxy)acetamido)heptanamido)ethyl) (methyl)carbamoyl)-3- hydroxyphenyl)isoxazole-3- carboxamide |
| 250 | LQ126- 112 | 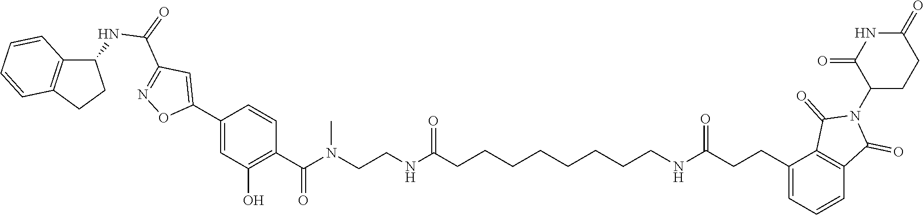 [SMILES: CN(CCNC(=O)CCCCCCCCNC(=O)CCc1cccc2c1C(=O)N(C1CCC(=O)NC1=O)C2=O)C(=O)c1ccc(-c2cc(C(=O)N[C@@H]3CCc4ccccc43)no2)cc1O] | N-((R)-2,3-dihydro-1H-inden-1-yl)- 5-(4-((2-(9-(2-((2-(2,6- dioxopiperidin-3-yl)-1,3- dioxoisoindolin-4-yl) oxy)acetamido)nonanamido)ethyl) (methyl)carbamoyl)-3- hydroxyphenyl)isoxazole-3- carboxamide |
| 251 | LQ126- 113 | 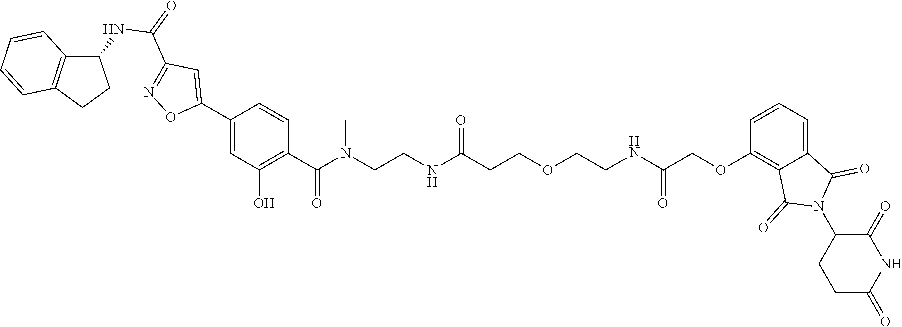 [SMILES: CN(CCNC(=O)CCOCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O] | N-((R)-2,3-dihydro-1H-inden-1-yl)- 5-(4-((2-(3-(2-(2-((2-(2,6- dioxopiperidin-3-yl)-1,3- dioxoisoindolin-4-yl) oxy)acetamido)ethoxy)propanami- do)ethyl)(methyl)carbamoyl)-3- hydroxyphenyl)isoxazole-3- carboxamide |
| 252 | LQ126- 114 | 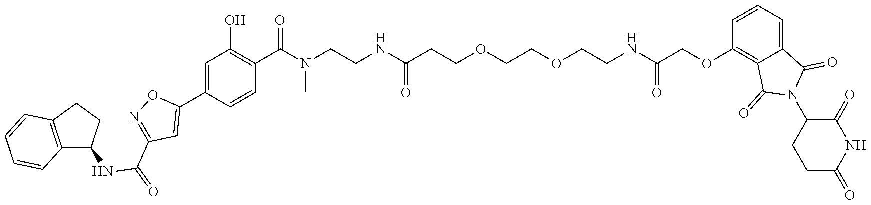 [SMILES: CN(CCNC(=O)CCOCCOCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O] | N-((R)-2,3-dihydro-1H-inden-1-yl)- 5-(4-((1-((2-(2,6-dioxopiperidin-3- yl)-1,3-dioxoisoindolin-4-yl)oxy)- 2,12-dioxo-6,9-dioxa-3,13- diazapentadecan-15- yl)(methyl)carbamoyl)-3- hydroxyphenyl)isoxazole-3 carboxamide |
| 253 | LQ126- 115 | 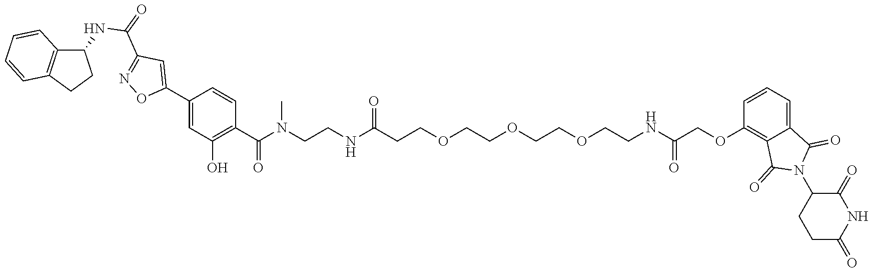 [SMILES: CN(CCNC(=O)CCOCCOCCOCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O] | N-((R)-2,3-dihydro-1H-inden-1-yl)- 5-(4-((1-((2-(2,6-dioxopiperidin-3- yl)-1,3-dioxoisoindolin-4-yl)oxy)- 2,15-dioxo-6,9,12-trioxa-3,16- diazaoctadecan-18- yl)(methyl)carbamoyl)-3- hydroxyphenyl)isoxazole-3- carboxamide |
| 254 | LQ126- 116 | 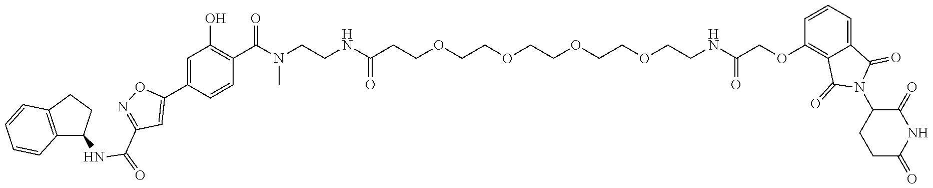 [SMILES: CN(CCNC(=O)CCOCCOCCOCCOCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O] | N-((R)-2,3-dihydro-1H-inden-1-yl)- 5-(4-((1-((2-(2,6-dioxopiperidin-3- yl)-1,3-dioxoisoindolin-4-yl)oxy)- 2,18-dioxo-6,9,12,15-tetraoxa-3.19- diazahenicosan-21- yl)(methyl)carbamoyl)-3- hydroxyphenyl)isoxazole-3- carboxamide |
| 255 | LQ126- 117 | 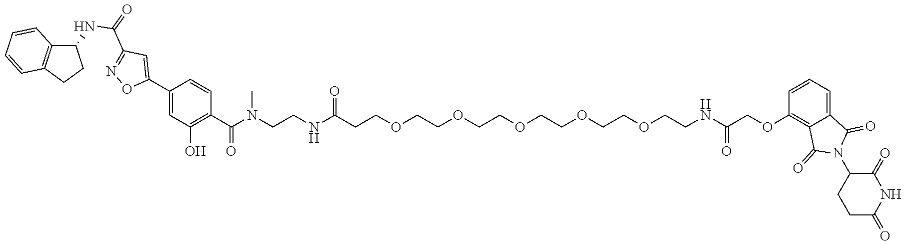 [SMILES: CN(CCNC(=O)CCOCCOCCOCCOCCOCCNC(=O)COc1cccc2c1C(=O)N(C1CCC(=O)NC1=O)C2=O)C(=O)c1ccc(-c2cc(C(=O)N[C@@H]3CCc4ccccc43)no2)cc1O] | N-((R)-2,3-dihydro-1H-inden-1-yl)- 5-(4-((1-((2-(2,6-dioxopiperidin-3- yl)-1,3-dioxoisoindolin-4-yl)oxy)- 2,21-dioxo-6,9,12,15,18-pentaoxa- 3,22-diazatetracosan-24- yl)(methyl)carbamoyl)-3- hydroxyphenyl)isoxazole-3- carboxamide |
| 256 | LQ126- 118 | 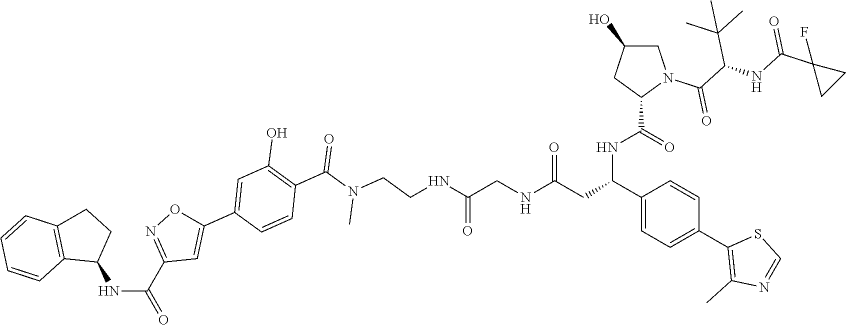 [SMILES: Cc1ncsc1-c1ccc([C@H](CC(=O)NCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1] | N-((R)-2,3-dihydro-1H-inden-1-yl)- 5-(4-((2-(2-((S)-3-((2S,4R)-1-((S)- 2-(1-fluorocyclopropane-1- carboxamido)-3,3- dimethylbutanoyl)-4- hydroxypyrrolidine-2- carboxamido)-3-(4-(4- methylthiazol-5- yl)phenyl)propanamido)acetamido) ethyl)(methyl)carbamoyl)-3- hydroxyphenyl)isoxazole-3- carboxamide |
| 257 | LQ126- 120 | 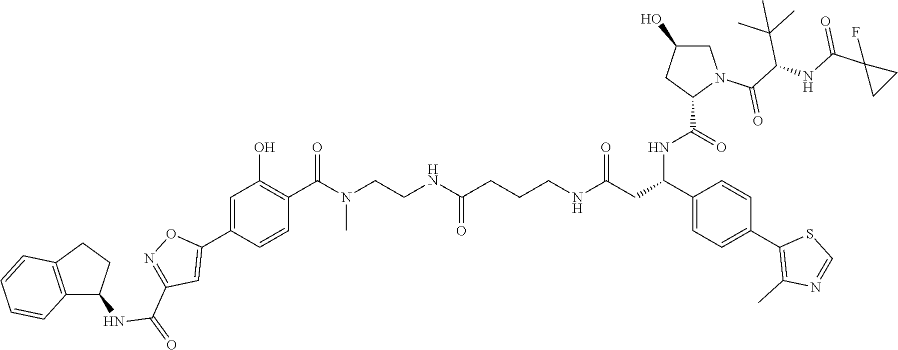 [SMILES: Cc1ncsc1-c1ccc([C@H](CC(=O)NCCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1] | N-((R)-2,3-dihydro-1H-inden-1-yl)- 5-(4-((2-(4-((S)-3-((2S,4R)-1-((S)- 2-(1-fluorocyclopropane-1- carboxamido)-3,3- dimethylbutanoyl)-4- hydroxypyrrolidine-2- carboxamido)-3-(4-(4- methylthiazol-5-yl) phenyl)propanamido)butanamido) ethyl)(methyl)carbamoyl)-3- hydroxyphenyl)isoxazole-3- carboxamide |
| 258 | LQ126- 121 | 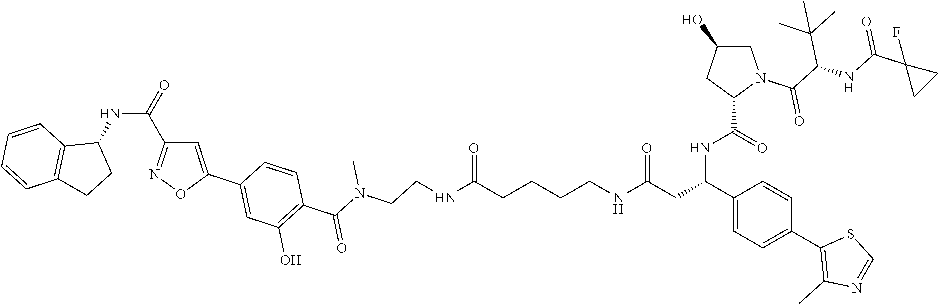 [SMILES: Cc1ncsc1-c1ccc([C@H](CC(=O)NCCCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1] | N-((R)-2,3-dihydro-1H-inden-1-yl)- 5-(4-((2-(5-((S)-3-((2S,4R)-1-((S)- 2-(1-fluorocyclopropane-1- carboxamido)-3,3- dimethylbutanoyl)-4- hydroxypyrrolidine-2- carboxamido)-3-(4-(4- methylthiazol-5-yl) phenyl)propanamido)pentanamido) ethyl)(methyl)carbamoyl)-3- hydroxyphenyl)isoxazole-3- carboxamide |
| 259 | LQ126- 122 | 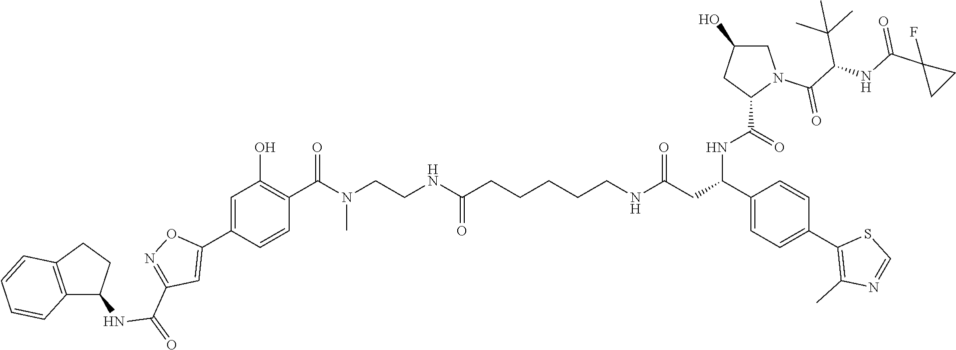 [SMILES: Cc1ncsc1-c1ccc([C@H](CC(=O)NCCCCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1] | N-((R)-2,3-dihydro-1H-inden-1-yl)- 5-(4-((2-(6-((S)-3-((2S,4R)-1-((S)- 2-(1-fluorocyclopropane-1- carboxamido)-3,3- dimethylbutanoyl)-4- hydroxypyrrolidine-2- carboxamido)-3-(4-(4- methylthiazol-5-yl) phenyl)propanamido)hexanamido) ethyl)(methyl)carbamoyl)-3- hydroxyphenyl)isoxazole-3- carboxamide |
| 260 | LQ126- 123 | 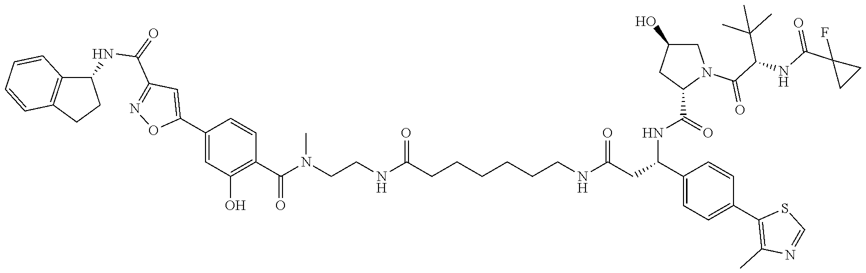 [SMILES: Cc1ncsc1-c1ccc([C@H](CC(=O)NCCCCCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1] | N-((R)-2,3-dihydro-1H-inden-1-yl)- 5-(4-((2-(7-((S)-3-((2S,4R)-1-((S)- 2-(1-fluorocyclopropane-1- carboxamido)-3,3- dimethylbutanoyl)-4- hydroxypyrrolidine-2- carboxamido)-3-(4-(4- methylthiazol-5-yl)phe- nyl)propanamido)heptanamido) ethyl)(methyl)carbamoyl)-3- hydroxyphenyl)isoxazole-3- carboxamide |
| 261 | LQ126- 124 | 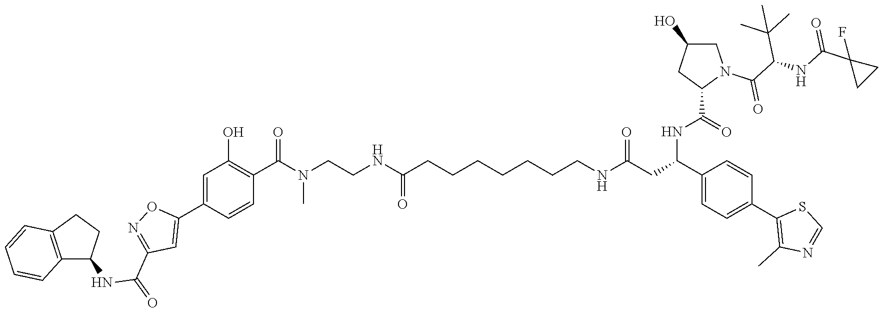 [SMILES: Cc1ncsc1-c1ccc([C@H](CC(=O)NCCCCCCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1] | N-((R)-2,3-dihydro-1H-inden-1-yl)- 5-(4-((2-(8-((S)-3-((2S,4R)-1-((S)- 2-(1-fluorocyclopropane-1- carboxamido)-3,3- dimethylbutanoyl)-4- hydroxypyrrolidine-2- carboxamido)-3-(4-(4- methylthiazol-5-yl) phenyl)propanamido)octanamido) ethyl)(methyl)carbamoyl)-3- hydroxyphenyl)isoxazole-3- carboxamide |
| 262 | LQ126- 125 | 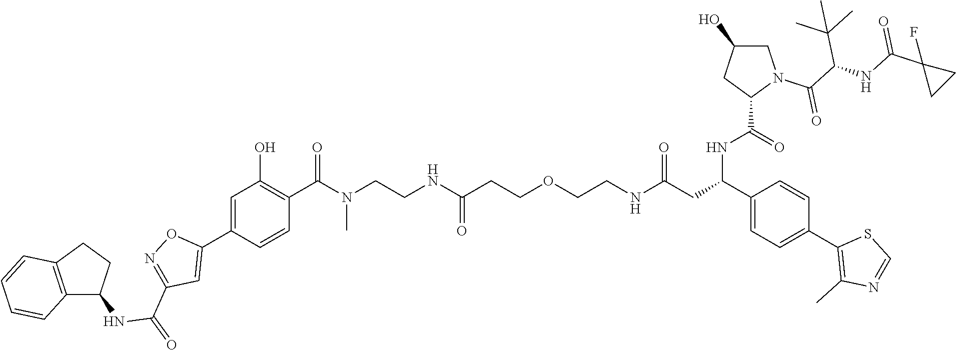 [SMILES: Cc1ncsc1-c1ccc([C@H](CC(=O)NCCOCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1] | N-((R)-2,3-dihydro-1H-inden-1-yl)- 5-(4-(((S)-1-((2S,4R)-1-((S)-2-(1- flurocyclopropane-1- carboxamido)-3,3- dimethylbutanoyl)-4- hydroxypyrrolidin-2-yl)-3-(4-(4- methylthiazol-5-yl)phenyl)-1,5,12- trioxo-9-oxa-2,6,13- triazapentadecan-15- yl)(methyl)carbamoyl)-3- hydroxyphenyl)isoxazole-3- carboxamide |
| 263 | LQ126- 126 | 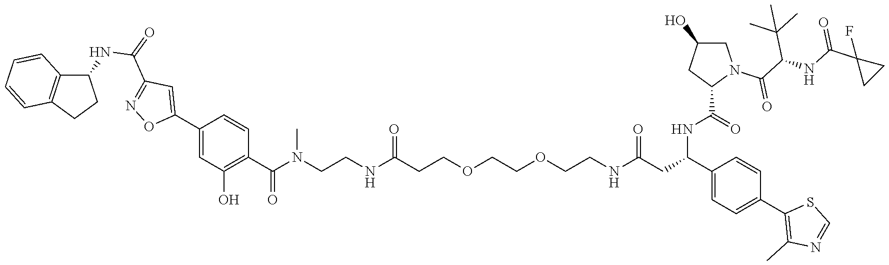 [SMILES: Cc1ncsc1-c1ccc([C@H](CC(=O)NCCOCCOCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1] | N-((R)-2,3-dihydro-1H-inden-1-yl)- 5-(4-(((S)-1-((2S,4R)-1-((S)-2-(1- fluorocyclopropane-1- carboxamido)-3,3- dimethylbutanoyl)-4- hydroxypyrrolidin-2-yl)-3-(4-(4- methylthiazol-5-yl)phenyl)-1,5,15- trioxo-9,12-dioxa-2,6,16- triazaoctadecan-18- yl)(methyl)carbamoyl)-3- hydroxyphenyl)isoxazole-3- carboxamide |
| 264 | LQ126- 127 | 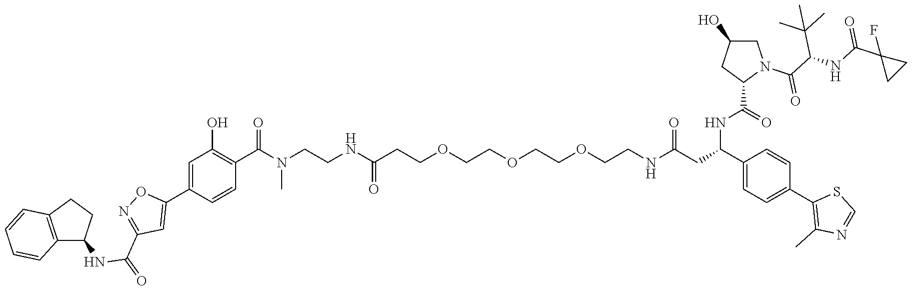 [SMILES: Cc1ncsc1-c1ccc([C@H](CC(=O)NCCOCCOCCOCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1] | N-((R)-2,3-dihydro-1H-inden-1-yl)- 5-(4-(((S)-1-((2S,4R)-1-((S)-2-(1- fluorocyclopropane-1- carboxamido)-3,3- dimethylbutanoyl)-4- hydroxypyrrolidin-2-yl)-3-(4-(4- methylthiazol-5-yl)phenyl)-1,5,18- trioxo-9,12,15-trioxa-2,6,19- triazahenicosan-21- yl)(methyl)carbamoyl)-3- hydroxyphenyl)isoxazole-3- carboxamide |
| 265 | LQ126- 128 | 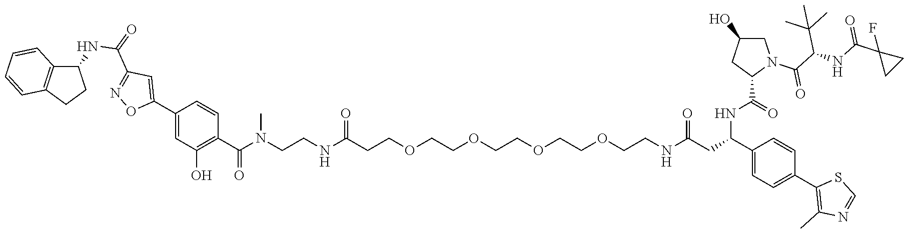 [SMILES: Cc1ncsc1-c1ccc([C@H](CC(=O)NCCOCCOCCOCCOCCC(=O)NCCN(C)C(=O)c2ccc(-c3cc(C(=O)N[C@@H]4CCc5ccccc54)no3)cc2O)NC(=O)[C@@H]2C[C@@H](O)CN2C(=O)[C@@H](NC(=O)C2(F)CC2)C(C)(C)C)cc1] | N-((R)-2,3-dihydro-1H-inden-1-yl)- 5-(4-(((S)-1-((2S,4R)-1-((S)-2-(1- fluorocyclopropane-1- carboxamido)-3,3- dimethylbutanoyl)-4- hydroxypyrrolidin-2-yl)-3-(4-(4- methylthiazol-5-yl)phenyl)-1,5,21- trioxo-9,12,15,18-tetraoxa-2,6,22- triazatetracosan-24- yl)(methyl)carbamoyl)-3- hydroxyphenyl)isoxazole-3- carboxamide |
| 266 | LQ126- 130 | 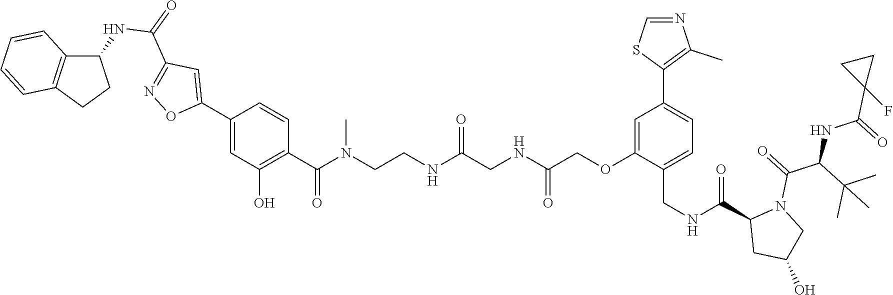 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCC(=O)NCCN(C)C(=O)c2ccc(-c3cc(C(=O)N[C@@H]4CCc5ccccc54)no3)cc2O)c1] | N-((R)-2,3-dihydro-1H-inden-1-yl)- 5-(4-((2-(2-(2-(2-(((2S,4R)-1-((S)- 2-(1-fluorocyclopropane-1- carboxamido)-3,3- dimethylbutanoyl)-4- hydroxypyrrolidine-2- carboxamido)methyl)-5-(4- methylthiazol-5-yl)phe- noxy)acetamido)acetamido)eth- yl)(methyl)carbamoyl)-3- hydroxyphenyl)isoxazole-3- carboxamide |
| 267 | LQ126- 168 | 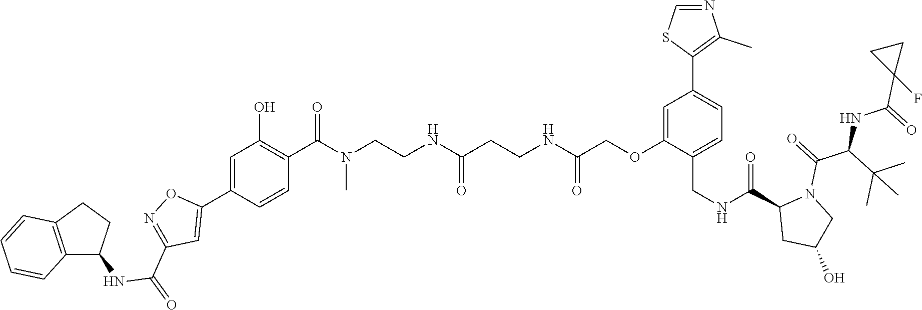 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCC(=O)NCCN(C)C(=O)c2ccc(-c3cc(C(=O)N[C@@H]4CCc5ccccc54)no3)cc2O)c1] | N-((R)-2,3-dihydro-1H-inden-1-yl)- 5-(4-((2-(3-(2-(2-(((2S,4R)-1-((S)- 2-(1-fluorocyclopropane-1- carboxamido)-3,3- dimethylbutanoyl)-4- hydroxypyrrolidine-2- carboxamido)methyl)-5-(4- methylthiazol-5-yl) phenoxy)acetamido)propanamido) ethyl)(methyl)carbamoyl)-3- hydroxyphenyl)isoxazole-3- carboxamide |
| 268 | LQ126- 170 | 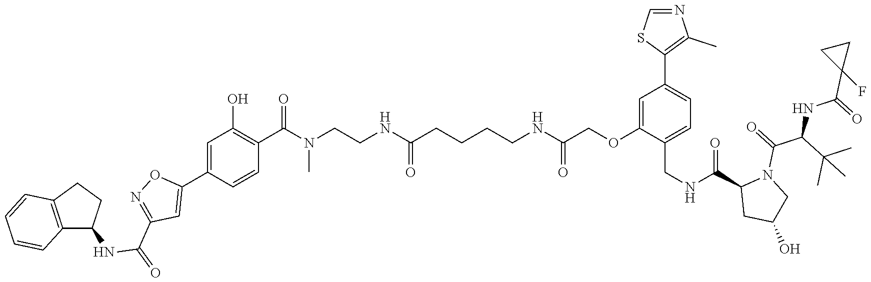 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCC(=O)NCCN(C)C(=O)c2ccc(-c3cc(C(=O)N[C@@H]4CCc5ccccc54)no3)cc2O)c1] | N-((R)-2,3-dihydro-1H-inden-1-yl)- 5-(4-((2-(5-(2-(2-(((2S,4R)-1-((S)- 2-(1-fluorocyclopropane-1- carboxamido)-3,3- dimethylbutanoyl)-4- hydroxypyrrolidine-2- carboxamido)methyl)-5-(4- methylthiazol-5-yl) phenoxy)acetamido)pentanamido) ethyl)(methyl)carbamoyl)-3- hydroxyphenyl)isoxazole-3- carboxamide |
| 269 | LQ126- 171 | 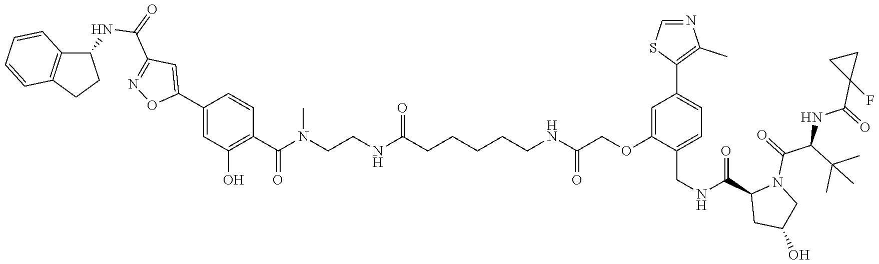 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCC(=O)NCCN(C)C(=O)c2ccc(-c3cc(C(=O)N[C@@H]4CCc5ccccc54)no3)cc2O)c1] | N-((R)-2,3-dihydro-1H-inden-1-yl)- 5-(4-((2-(6-(2-(2-(((2S,4R)-1-((S)- 2-(1-fluorocyclopropane-1- carboxamido)-3,3- dimethylbutanoyl)-4- hydroxypyrrolidine-2- carboxamido)methyl)-5-(4- methylthiazol-5- yl)phenoxy)acetamido)hexanamido) ethyl)(methyl)carbamoyl)-3- hydroxyphenyl)isoxazole-3- carboxamide |
| 270 | LQ126- 172 | 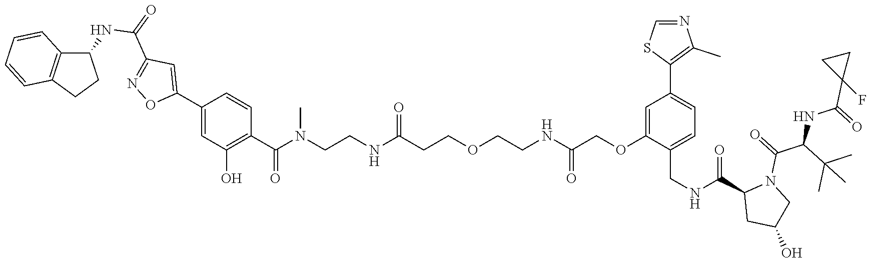 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCC(=O)NCCN(C)C(=O)c2ccc(-c3cc(C(=O)N[C@@H]4CCc5ccccc54)no3)cc2O)c1] | N-((R)-2,3-dihydro-1H-inden-1-yl)- 5-(4-((2-(3-(2-(2-(2-(((2S,4R)-1- ((S)-2-(1-fluorocylcopropane-1- carboxamido)-3,3- dimethylbutanoyl)-4- hydroxypyrrolidine-2- carboxamido)methyl)-5-(4- methylthiazol-5-yl) phenoxy)acetamido)ethoxy)propa- namido)ethyl)(methyl)carbamoyl)- 3-hydroxyphenyl)isoxazole-3- carboxamide |
| 271 | LQ126- 173 | 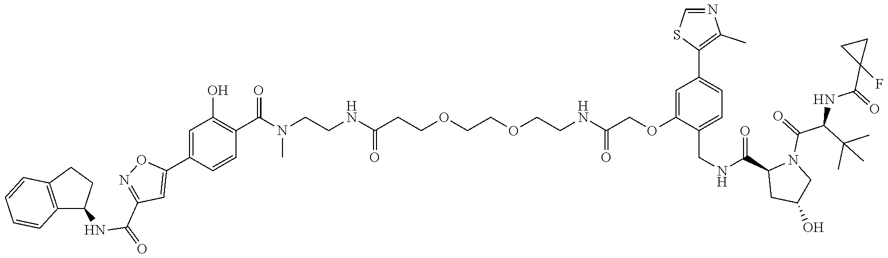 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCOCCC(=O)NCCN(C)C(=O)c2ccc(-c3cc(C(=O)N[C@@H]4CCc5ccccc54)no3)cc2O)c1] | N-((R)-2,3-dihydro-1H-inden-1-yl)- 5-(4-((1-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5-yl)phenoxy)-2,12- dioxo-6,9-dioxa-3,13- diazapentadecan-15- yl)(methyl)carbamoyl)-3- hydroxyphenyl)(isoxazole-3- carboxamide |
| 272 | LQ126- 174 | 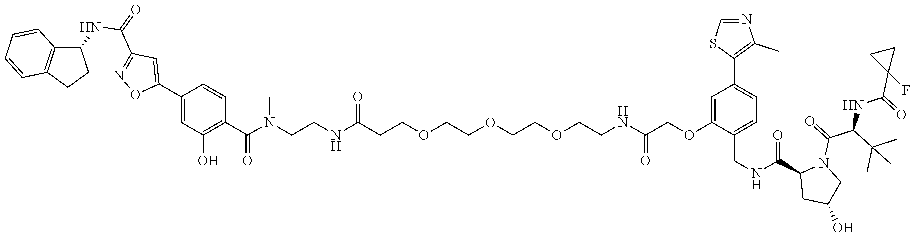 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCOCCOCCC(=O)NCCN(C)C(=O)c2ccc(-c3cc(C(=O)N[C@@H]4CCc5ccccc54)no3)cc2O)c1] | N-((R)-2,3-dihydro-1H-inden-1-yl)- 5-(4-((1-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5-yl)phenoxy)-2,15- dioxo-6,9,12-trioxa-3,16- diazaoctadecan-18- yl)(methyl)carbamoyl)-3- hydroxyphenyl)isoxazole-3- carboxamide |
| 273 | LQ126- 175 | 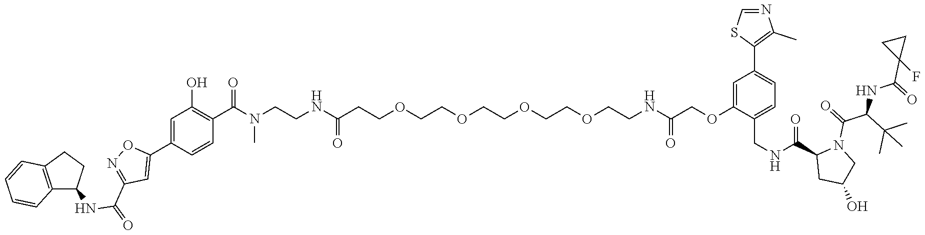 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCOCCOCCOCCC(=O)NCCN(C)C(=O)c2ccc(-c3cc(C(=O)N[C@@H]4CCc5ccccc54)no3)cc2O)c1] | N-((R)-2,3-dihydro-1H-inden-1-yl)- 5-(4-((1-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5-yl)phenoxy)-2,18- dioxo-6,9,12,15-tetraoxa-3,19- diazahenicosan-21- yl)(methyl)carbamoyl)-3- hydroxyphenyl)isoxazole-3- carboxamide |
| 274 | LQ126- 176 | 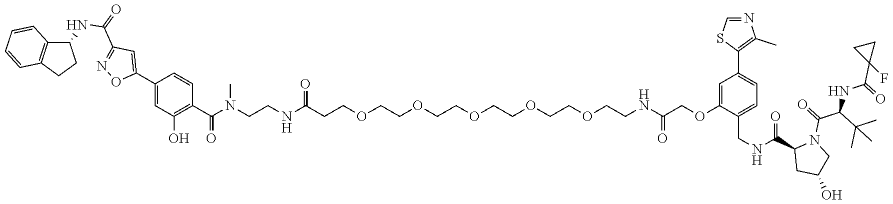 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCOCCOCCOCCOCCC(=O)NCCN(C)C(=O)c2ccc(-c3cc(C(=O)N[C@@H]4CCc5ccccc54)no3)cc2O)c1] | N-((R)-2,3-dihydro-1H-inden-1-yl)- 5-(4-((1-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5-yl)phenoxy)-2,21- dioxo-6,9,12,15,18-pentaoxa-3,22- diazatetracosan-24- yl)(methyl)carbamoyl)-3- hydroxyphenyl)isoxazole-3- carboxamide |
| 276 | LQ126- 177 | 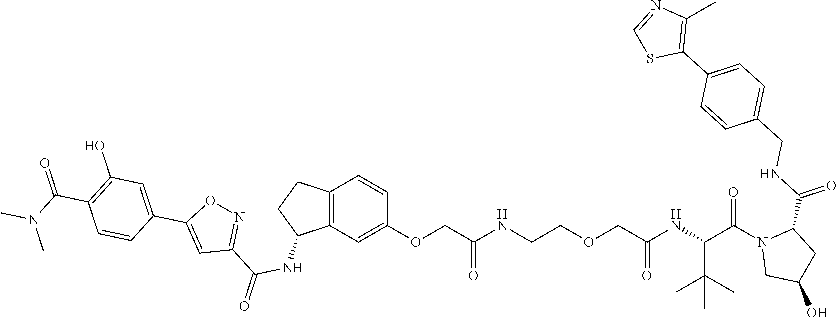 [SMILES: Cc1ncsc1-c1ccc(CNC(=O)[C@@H]2C[C@@H](O)CN2C(=O)[C@@H](NC(=O)COCCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(2-((2-(2- (((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-2- oxoethoxy)ethyl)amino)-2- oxoethoxy)-2,3-dihydro-1H-inden- 1-yl)isoxazole-3-carboxamide |
| 277 | LQ126- 178 | 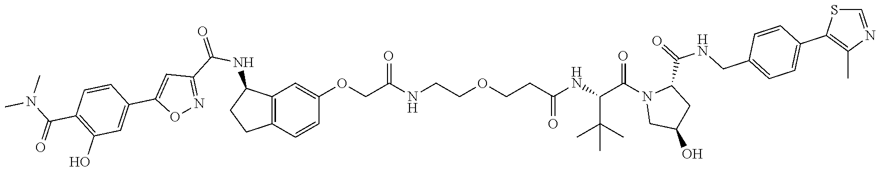 [SMILES: Cc1ncsc1-c1ccc(CNC(=O)[C@@H]2C[C@@H](O)CN2C(=O)[C@@H](NC(=O)CCOCCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(2-((2-(3- (((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-3- oxopropoxy)ethyl)amino)-2- oxoethoxy)-2,3-dihydro-1H-inden- 1-yl)isoxazole-3-carboxamide |
| 278 | LQ126- 180 | 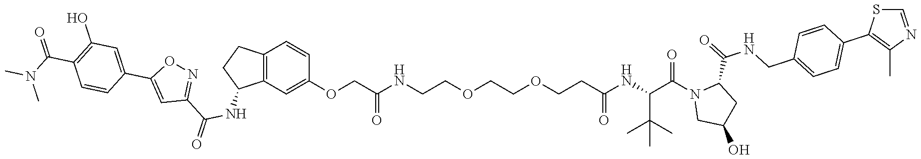 [SMILES: Cc1ncsc1-c1ccc(CNC(=O)[C@@H]2C[C@@H](O)CN2C(=O)[C@@H](NC(=O)CCOCCOCCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(((S)-14- ((2S,4R)-4-hydroxy-2-((4-(4- methylthiazol-5- yl)benzyl)carbamoyl)pyrrolidine-1- carbonyl)-15,15-dimethyl-2,12- dioxo-6,9-dioxa-3,13- diazahexadecyl)oxy)-2,3-dihydro- 1H-inden-1-yl)isoxazole-3- carboxamide |
| 279 | LQ126- 181 | 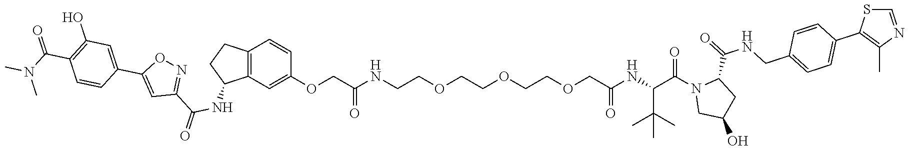 [SMILES: Cc1ncsc1-c1ccc(CNC(=O)[C@@H]2C[C@@H](O)CN2C(=O)[C@@H](NC(=O)COCCOCCOCCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(((S)-16- ((2S,4R)-4-hydroxy-2-((4-(4- methylthiazol-5- yl)benzyl)carbamoyl)pyrrolidine-1- carbonyl)-17,17-dimethyl-12,14- dioxo-6,9,12-trioxa-3,15- diazaoctadecyl)oxy)-2,3-dihydro- 1H-inden-1-yl)isoxazole-3- carboxamide |
| 280 | LQ126- 182 | 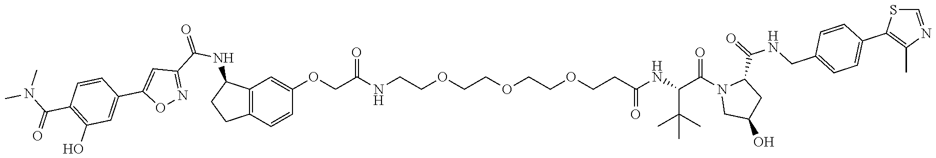 [SMILES: Cc1ncsc1-c1ccc(CNC(=O)[C@@H]2C[C@@H](O)CN2C(=O)[C@@H](NC(=O)CCOCCOCCOCCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(((S)-17- ((2S,4R)-4-hydroxy-2-((4-(4- methylthiazol-5- yl)benzyl)carbamoyl)pyrrolidine-1- carbonyl)-18,18-dimethyl-2,15- dioxo-6,9,12-trioxa-3,16- diazanonadecyl)oxy)-2,3-dihydro- 1H-inden-1-yl)isoxazole-3- carboxamide |
| 281 | LQ126- 183 | 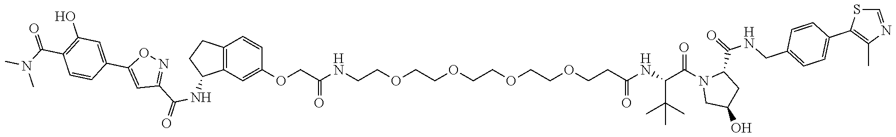 [SMILES: Cc1ncsc1-c1ccc(CNC(=O)[C@@H]2C[C@@H](O)CN2C(=O)[C@@H](NC(=O)CCOCCOCCOCCOCCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(((S)-20- ((2S,4R)-4-hydroxy-2-((4-(4- methylthiazol-5- yl)benzyl)carbamoyl)pyrrolidine-1- carbonyl)-21,21-dimethyl-2,18- dioxo-6,9,12,15-tetraoxa-3,19- diazadocosyl)oxy)-2,3-dihydro-1H- inden-1-yl)isoxazole-3- |
| | | | carboxamide |
| 282 | LQ126- 184 | 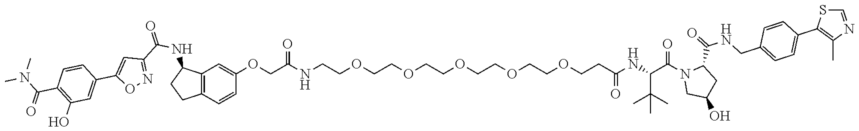 [SMILES: Cc1ncsc1-c1ccc(CNC(=O)[C@@H]2C[C@@H](O)CN2C(=O)[C@@H](NC(=O)CCOCCOCCOCCOCCOCCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(((S)-23- ((2S,4R)-4-hydroxy-2-((4-(4- methylthiazol-5- yl)benzyl)carbamoyl)pyrrolidine-1- carbonyl)-24,24-dimethyl-2,21- dioxo-6,9,12,15,18-pentaoxa-3,22- diazapentacosyl)oxy)-2,3-dihydro- |
| | | | 1H-inden-1-yl)isoxazole-3- |
| | | | carboxamide |
| 283 | LQ126- 185 | 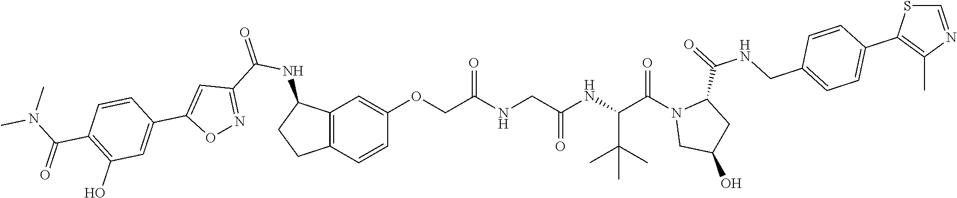 [SMILES: Cc1ncsc1-c1ccc(CNC(=O)[C@@H]2C[C@@H](O)CN2C(=O)[C@@H](NC(=O)CNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(2-((2- (((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-2-oxoethyl)amino)-2- oxoethoxy)-2,3-dihydro-1H-inden- 1-yl)isoxazole-3-carboxamide |
| 284 | LQ126- 186 | 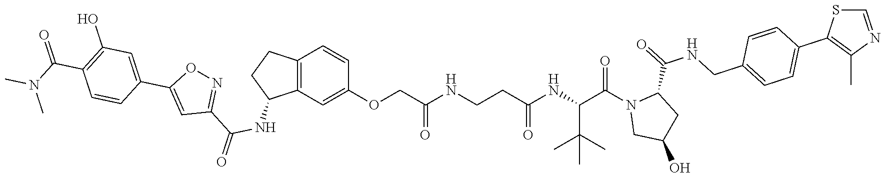 [SMILES: Cc1ncsc1-c1ccc(CNC(=O)[C@@H]2C[C@@H](O)CN2C(=O)[C@@H](NC(=O)CCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(2-((3- (((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-3-oxopropyl)amino)-2- oxoethoxy)-2,3-dihydro-1H-inden- 1-yl)isoxazole-3-carboxamide |
| 285 | LQ141- 1 | 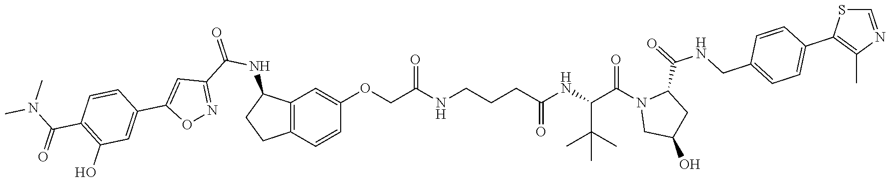 [SMILES: Cc1ncsc1-c1ccc(CNC(=O)[C@@H]2C[C@@H](O)CN2C(=O)[C@@H](NC(=O)CCCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(2-((4- (((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-4-oxobutyl)amino)-2- oxoethoxy)-2,3-dihydro-1H-inden- 1-yl)isoxazole-3-carboxamide |
| 286 | LQ141- 2 | 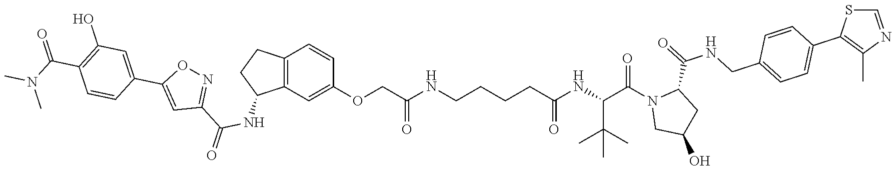 [SMILES: Cc1ncsc1-c1ccc(CNC(=O)[C@@H]2C[C@@H](O)CN2C(=O)[C@@H](NC(=O)CCCCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(2-((5- (((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-5-oxopentyl)amino)-2- oxoethoxy)-2,3-dihydro-1H-inden- 1-yl)isoxazole-3-carboxamide |
| 287 | LQ141- 3 | 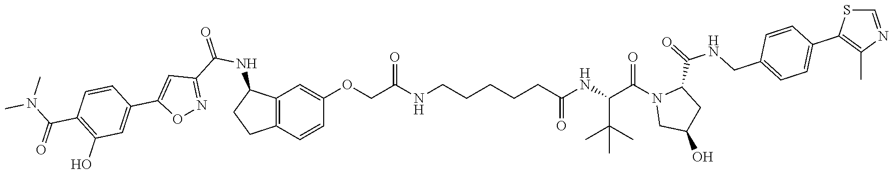 [SMILES: Cc1ncsc1-c1ccc(CNC(=O)[C@@H]2C[C@@H](O)CN2C(=O)[C@@H](NC(=O)CCCCCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(2-((6- (((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-6-oxohexyl)amino)-2- oxoethoxy)-2,3-dihydro-1H-inden- 1-yl)isoxazole-3-carboxamide |
| 288 | LQ141- 4 | 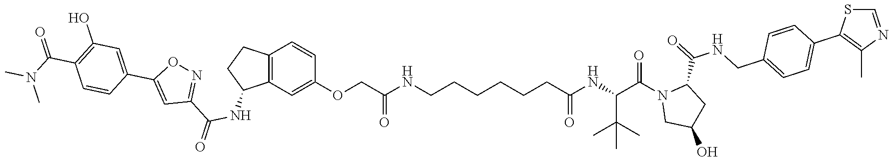 [SMILES: Cc1ncsc1-c1ccc(CNC(=O)[C@@H]2C[C@@H](O)CN2C(=O)[C@@H](NC(=O)CCCCCCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(2-((7- (((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-7-oxoheptyl)amino)-2- oxoethoxy)-2,3-dihydro-1H-inden- 1-yl)isoxazole-3-carboxamide |
| 289 | LQ141- 5 | 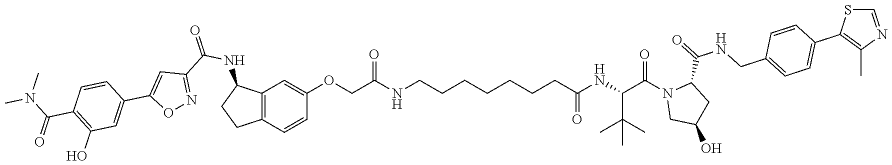 [SMILES: Cc1ncsc1-c1ccc(CNC(=O)[C@@H]2C[C@@H](O)CN2C(=O)[C@@H](NC(=O)CCCCCCCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(2-((8- (((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-8-oxooctyl)amino)-2- oxoethoxy)-2,3-dihydro-1H-inden- 1-yl)isoxazole-3-carboxamide |
| 290 | LQ141- 6 | 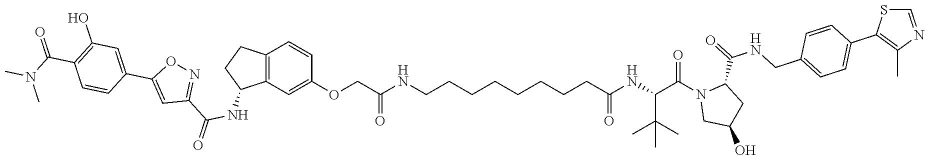 [SMILES: Cc1ncsc1-c1ccc(CNC(=O)[C@@H]2C[C@@H](O)CN2C(=O)[C@@H](NC(=O)CCCCCCCCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(2-((9- (((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-9-oxononyl)amino)-2- oxoethoxy)-2,3-dihydro-1H-inden- 1-yl)isoxazole-3-carboxamide |
| 291 | LQ141- 7 | 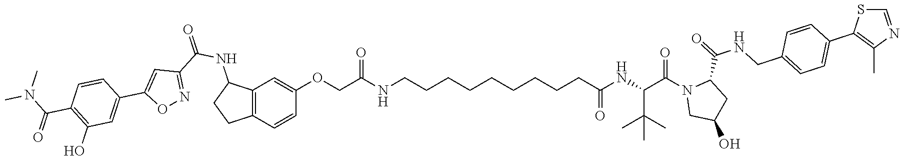 [SMILES: Cc1ncsc1-c1ccc(CNC(=O)[C@@H]2C[C@@H](O)CN2C(=O)[C@@H](NC(=O)CCCCCCCCCNC(=O)COc2ccc3c(c2)C(NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(2-((10- (((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-10-oxodecyl)amino)-2- oxoethoxy)-2,3-dihydro-1H-inden- 3-yl)isoxazole-3-carboxamide |
| 292 | LQ141- 8 | 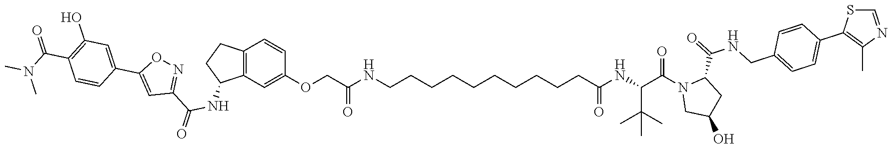 [SMILES: Cc1ncsc1-c1ccc(CNC(=O)[C@@H]2C[C@@H](O)CN2C(=O)[C@@H](NC(=O)CCCCCCCCCCNC(=O)COc2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-(2-((11- (((S)-1-((2S,4R)-4-hydroxy-2-((4- (4-methylthiazol-5- yl)benzyl)carbamoyl)pyrrolidin-1- yl)-3,3-dimethyl-1-oxobutan-2-yl) amino)-11-oxoundecyl)amino)-2- oxoethoxy)-2,3-dihydro-1H-inden- 1-yl)isoxazole-3-carboxamide |
| 293 | LQ141- 9 | 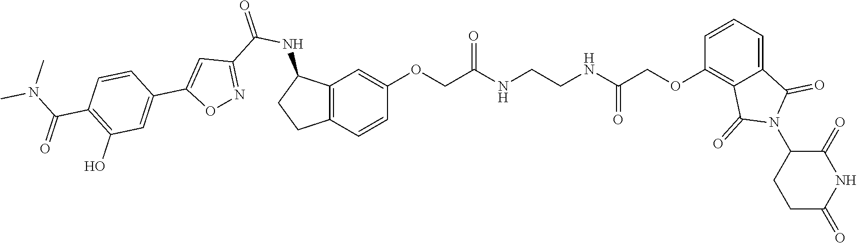 [SMILES: CN(C)C(=O)c1ccc(-c2cc(C(=O)N[C@@H]3CCc4ccc(OCC(=O)NCCNC(=O)COc5cccc6c5C(=O)N(C5CCC(=O)NC5=O)C6=O)cc43)no2)cc1O] | 5-(4-(dimethylcarbamoyl)-3-hy- droxyphenyl)-N-((1R)-6-(2-((2-(2- ((2-(2,6-dioxopiperidin-3-yl)-1,3- dioxoisoindolin-4- yl)oxy)acetamido)ethyl)amino)-2- oxoethoxy)-2,3-dihydro-1H-inden- 1-yl)isoxazole-3-carboxamide |
| 294 | LQ141- 10 | 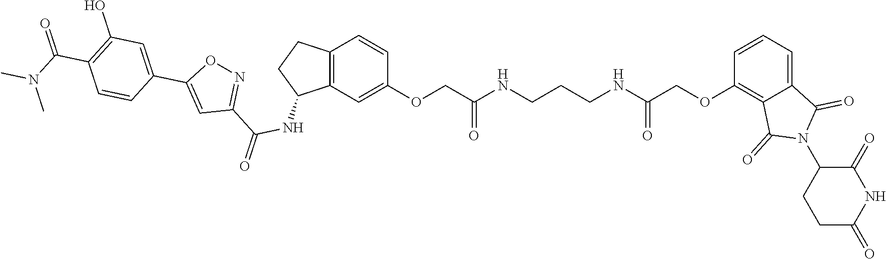 [SMILES: CN(C)C(=O)c1ccc(-c2cc(C(=O)N[C@@H]3CCc4ccc(OCC(=O)NCCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-(2-((3- (2-((2-(2,6-dioxopiperidin-3-yl)- 1,3-dioxoisoindolin-4- yl)oxy)acetamido)propyl)amino)-2- oxoethoxy)-2,3-dihydro-1H-inden- 1-yl)isoxazole-3-carboxamide |
| 295 | LQ141- 11 | 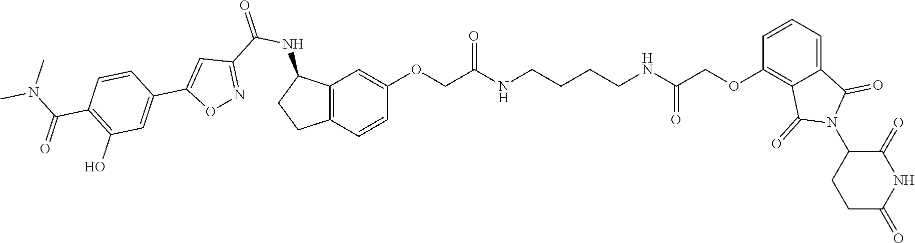 [SMILES: CN(C)C(=O)c1ccc(-c2cc(C(=O)N[C@@H]3CCc4ccc(OCC(=O)NCCCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-(2-((4- (2-((2-(2,6-dioxopiperidin-3-yl)- 1,3-dioxoisoindolin-4- yl)oxy)acetamido)butyl)amino)-2- oxoethoxy)-2,3-dihydro-1H-inden- 1-yl)isoxazole-3-carboxamide |
| 296 | LQ141- 12 | 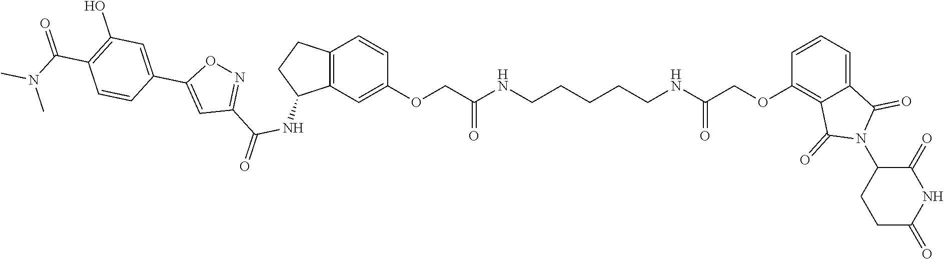 [SMILES: CN(C)C(=O)c1ccc(-c2cc(C(=O)N[C@@H]3CCc4ccc(OCC(=O)NCCCCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-(2-((5- (2-((2-(2,6-dioxopiperidin- 3-yl)-1,3-dioxoisoindolin-4- yl)oxy)acetamido)pentyl)amino)-2- oxoethoxy)-2,3-dihydro-1H-inden- 1-yl)isoxazole-3-carboxamide |
| 297 | LQ141- 13 | 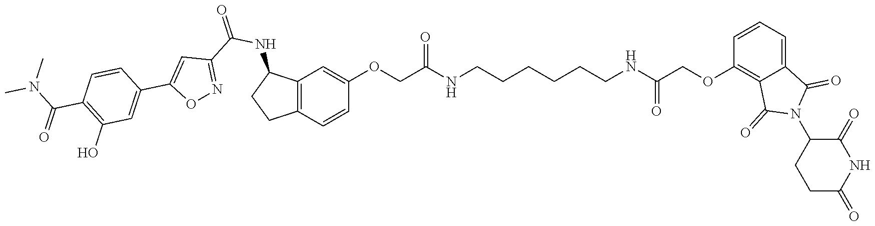 [SMILES: CN(C)C(=O)c1ccc(-c2cc(C(=O)N[C@@H]3CCc4ccc(OCC(=O)NCCCCCCNC(=O)COc5cccc6c5C(=O)N(C5CCC(=O)NC5=O)C6=O)cc43)no2)cc1O] | 5-(4-(dimethylcarbamoyl)-3-hy- droxyphenyl)-N-((1R)-6-(2-((6-(2- ((2-(2,6-dioxopiperidin-3-yl)-1,3- dioxoisoindolin-4- yl)oxy)acetamido)hexyl)amino)-2- oxoethoxy)-2,3-dihydro-1H-inden- 1-yl)isoxazole-3-carboxamide |
| 298 | LQ141- 14 | 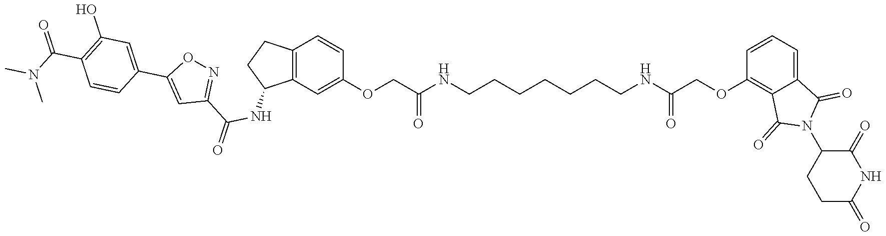 [SMILES: CN(C)C(=O)c1ccc(-c2cc(C(=O)N[C@@H]3CCc4ccc(OCC(=O)NCCCCCCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-(2-((7- (2-((2-(2,6-dioxopiperidin-3- yl)-1,3-dioxoisoindolin-4- yl)oxy)acetamido)heptyl)amino)-2- oxoethoxy)-2,3-dihydro-1H-inden- 1-yl)isoxazole-3-carboxamide |
| 299 | LQ141- 15 | 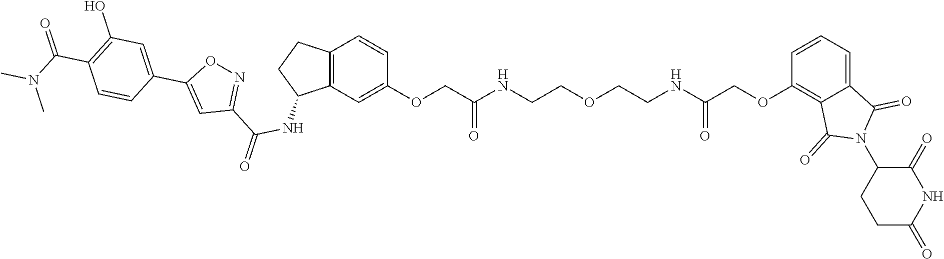 [SMILES: CN(C)C(=O)c1ccc(-c2cc(C(=O)N[C@@H]3CCc4ccc(OCC(=O)NCCOCCNC(=O)COc5cccc6c5C(=O)N(C5CCC(=O)NC5=O)C6=O)cc43)no2)cc1O] | 5-(4-(dimethylcarbamoyl)-3-hy- droxyphenyl)-N-((1R)-6-(2-((2-(2- (2-((2-(2,6-dioxopiperidin-3-yl)- 1,3-dioxoisoindolin-4-yl)oxy)ace- tamido)ethoxy)ethyl)ami- no)-2-oxoethoxy)-2,3-dihydro- 1H-inden-1-yl)isoxazole-3- carboxamide |
| 300 | LQ141- 16 | 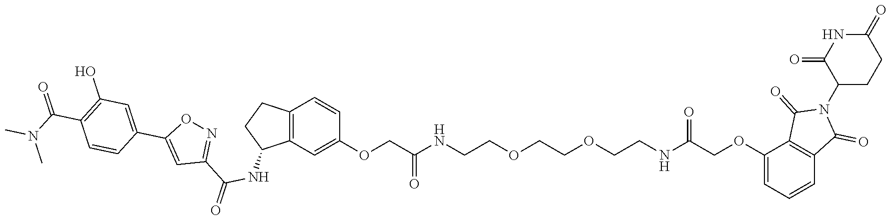 [SMILES: CN(C)C(=O)c1ccc(-c2cc(C(=O)N[C@@H]3CCc4ccc(OCC(=O)NCCOCCOCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-((14- ((2-(2,6-dioxopiperidin-3-yl)-1,3- dioxoisoindolin-4-yl)oxy)-2,13- dioxo-6,9-dioxa-3,12- diazatetradecyl)oxy)-2,3-dihydro- 1H-inden-1-yl)isoxazole-3- carboxamide |
| 301 | LQ141- 17 | 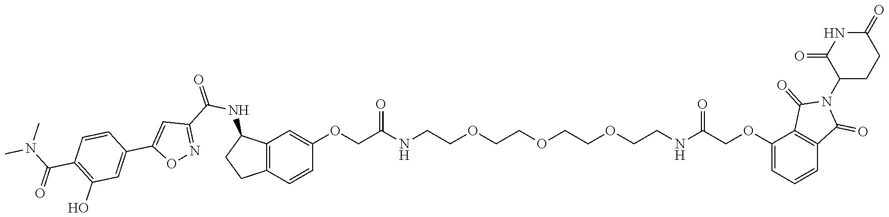 [SMILES: CN(C)C(=O)c1ccc(-c2cc(C(=O)N[C@@H]3CCc4ccc(OCC(=O)NCCOCCOCCOCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-((17- ((2-(2,6-dioxopiperidin-3-yl)-1,3- dioxoisoindolin-4-yl)oxy)-2,16- dioxo-6,9,12-trioxa-3,15- diazaheptadecyl)oxy)-2,3-dihydro- 1H-inden-1-yl)isoxazole-3- carboxamide |
| 302 | LQ141- 18 | 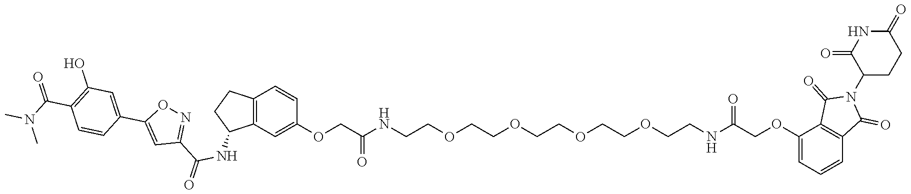 [SMILES: CN(C)C(=O)c1ccc(-c2cc(C(=O)N[C@@H]3CCc4ccc(OCC(=O)NCCOCCOCCOCCOCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-((20-((2- (2,6-dioxopiperidin-3-yl)-1,3- dioxoisoindolin-4-yl)oxy)-2,19- dioxo-6,9,12,15-tetraoxa-3,18- diazaicosyl)oxy)-2,3-dihydro-1H- inden-1-yl)isoxazole-3- carboxamide |
| 303 | LQ141- 19 | 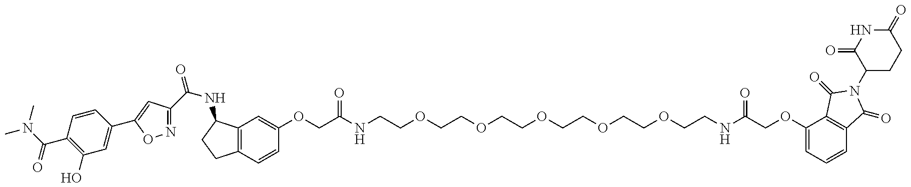 [SMILES: CN(C)C(=O)c1ccc(-c2cc(C(=O)N[C@@H]3CCc4ccc(OCC(=O)NCCOCCOCCOCCOCCOCCNC(=O)COc5cccc6c5C(=O)N(C5CCC(=O)NC5=O)C6=O)cc43)no2)cc1O] | 5-(4-(dimethylcarbamoyl)-3-hy- droxyphenyl)-N-((1R)-6-((23-((2- (2,6-dioxopiperidin-3-yl)-1,3- dioxoisoindolin-4-yl)oxy)-2,22- dioxo-6,9,12,15,18-pentaoxa-3,21- diazatricosyl)oxy)-2,3-dihydro-1H- inden-1-yl)isoxazole-3- carboxamide |
| 304 | LQ141- 20 | 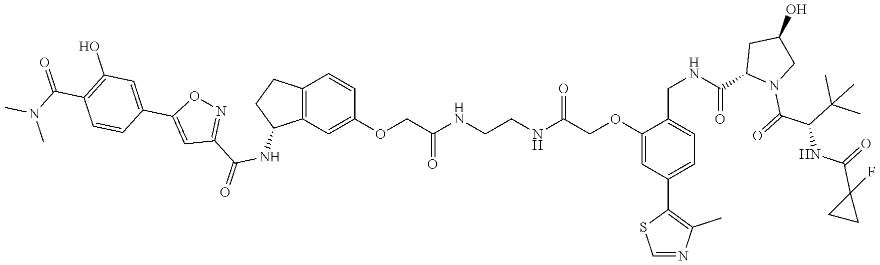 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCNC(=O)COc2ccc3c(c2)[C@H](NC(=O)c2cc(-c4ccc(C(=O)N(C)C)c(O)c4)on2)CC3)c1] | 5-(4-(dimethylcarbamoyl)-3- hydroxyphenyl)-N-((R)-6-(2-((2- (2-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5-yl) phenoxy)acetamido)ethyl)amino)- 2-oxoethoxy)-2,3-dihydro-1H- inden-1-yl)isoxazole-3- carboxamide |
| 305 | LQ141- 21 | 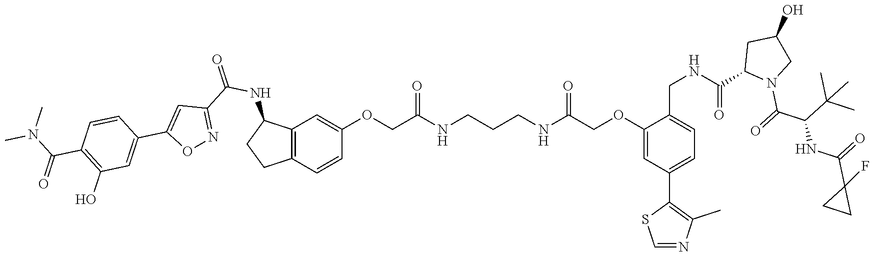 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCNC(=O)COc2ccc3c(c2)[C@H](NC(=O)c2cc(-c4ccc(C(=O)N(C)C)c(O)c4)on2)CC3)c1] | 5-(4-(dimethylcarbamoyl)-3- hydroxyphenyl)-N-((R)-6-(2-((3- (2-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5-yl)phe- noxy)acetamido)propyl)amino)- 2-oxoethoxy)-2,3-dihydro-1H- inden-1-yl)isoxazole-3- carboxamide |
| 306 | LQ141- 22 | 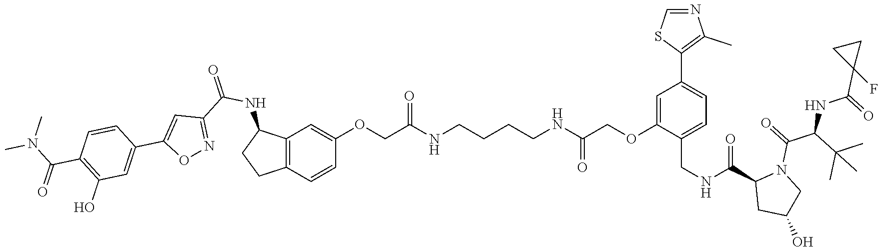 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCNC(=O)COc2ccc3c(c2)[C@H](NC(=O)c2cc(-c4ccc(C(=O)N(C)C)c(O)c4)on2)CC3)c1] | 5-(4-(dimethylcarbamoyl)-3- hydroxyphenyl)-N-((R)-6-(2-((4- (2-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5-yl) phenoxy)acetamido)butyl)amino)- 2-oxoethoxy)-2,3-dihydro-1H- inden-1-yl)isoxazole-3- carboxamide |
| 307 | LQ141- 24 | 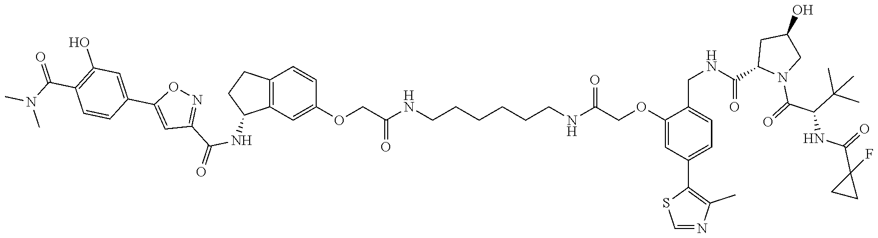 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCNC(=O)COc2ccc3c(c2)[C@H](NC(=O)c2cc(-c4ccc(C(=O)N(C)C)c(O)c4)on2)CC3)c1] | 5-(4-(dimethylcarbamoyl)-3- hydroxyphenyl)-N-((R)-6-(2-((6-(2- (2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5-yl) phenoxy)acetamido)hexyl)amino)- 2-oxoethoxy)-2,3-dihydro-1H- inden-1-yl)isoxazole-3- carboxamide |
| 308 | LQ141- 26 | 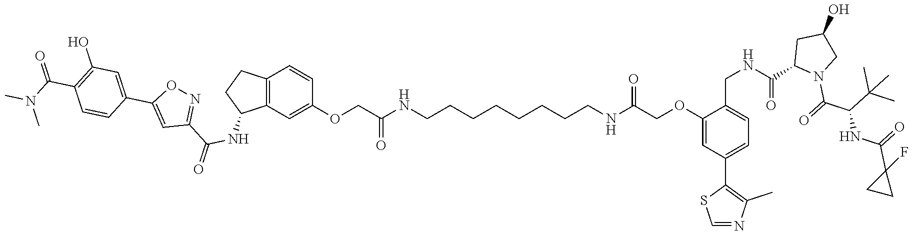 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCCCCNC(=O)COc2ccc3c(c2)[C@H](NC(=O)c2cc(-c4ccc(C(=O)N(C)C)c(O)c4)on2)CC3)c1] | 5-(4-(dimethylcarbamoyl)-3- hydroxyphenyl)-N-((R)-6-(2-((8- (2-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5-yl) phenoxy)acetamido)octyl)amino)- 2-oxoethoxy)-2,3-dihydro-1H- inden-1-yl)isoxazole-3- carboxamide |
| 309 | LQ141- 27 | 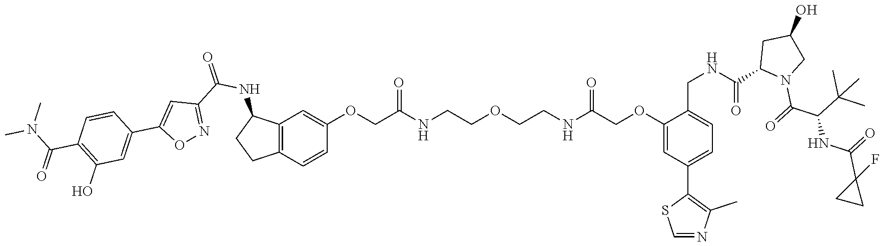 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCNC(=O)COc2ccc3c(c2)[C@H](NC(=O)c2cc(-c4ccc(C(=O)N(C)C)c(O)c4)on2)CC3)c1] | 5-(4-(dimethylcarbamoyl)-3- hydroxyphenyl)-N-((R)-6-(2-((2-(2- (2-(2-(((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5-yl) phenoxy)acetamido)ethoxy)ethyl) amino)-2-oxoethoxy)-2,3-dihydro- 1H-inden-1-yl)isoxazole-3- carboxamide |
| 310 | LQ141- 28 | 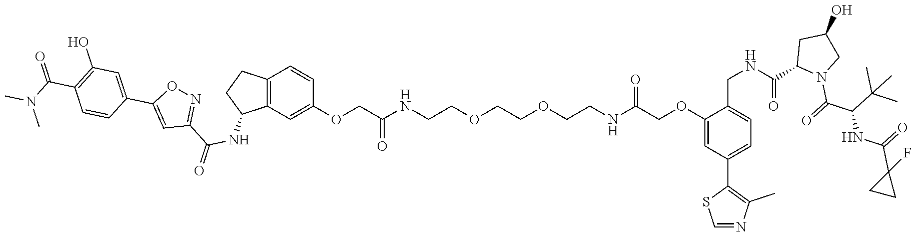 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCOCCNC(=O)COc2ccc3c(c2)[C@H](NC(=O)c2cc(-c4ccc(C(=O)N(C)C)c(O)c4)on2)CC3)c1] | 5-(4-(dimethylcarbamoyl)-3- hydroxyphenyl)-N-((R)-6-((14-(2- (((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5-yl)phenoxy)-2,13- dioxo-6,9-dioxa-3,12- diazatetradecyl)oxy)-2,3-dihydro- 1H-inden-1-yl)isoxazole-3- carboxamide |
| 311 | LQ141- 29 | 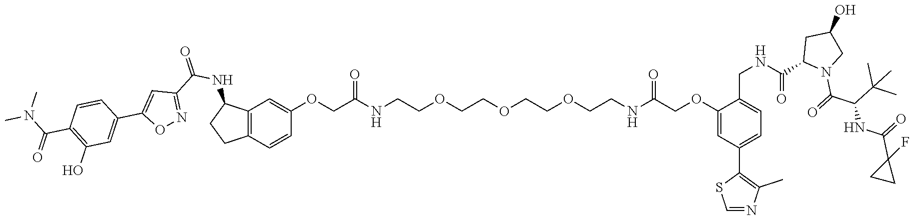 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCOCCOCCOCCNC(=O)COc2ccc3c(c2)[C@H](NC(=O)c2cc(-c4ccc(C(=O)N(C)C)c(O)c4)on2)CC3)c1] | 5-(4-(dimethylcarbamoyl)-3- hydroxyphenyl)-N-((R)-6-((17-(2- (((2S,4R)-1-((S)-2-(1- fluorocyclopropane-1- carboxamido)-3,3- dimethylbutanoyl)-4- hydroxypyrrolidine-2- carboxamido)methyl)-5-(4- methylthiazol-5-yl)phenoxy)-2,16- dioxo-6,9,12-trioxa-3,15- diazaheptadecyl)oxy)-2,3-dihydro- 1H-inden-1-yl)isoxazole-3- carboxamide |
| 312 | LQ141- 33 | 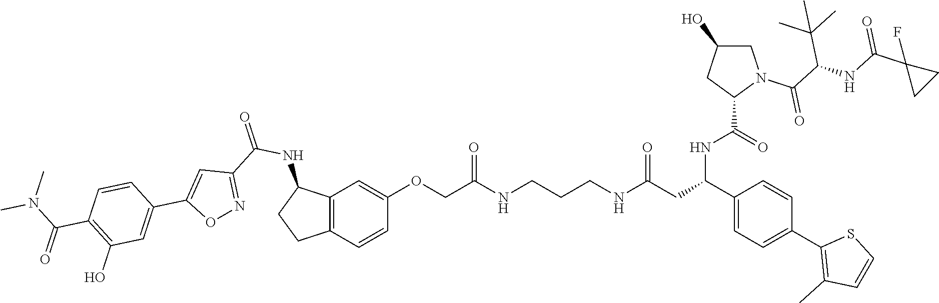 [SMILES: Cc1ccsc1-c1ccc([C@H](CC(=O)NCCCNC(=O)COc2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-(2-((3- ((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5-yl) phenyl)propanamido)propyl)ami- no)-2-oxoethoxy)-2,3-dihydro-1H- inden-1-yl)isoxazole-3- carboxamide |
| 313 | LQ141- 36 | 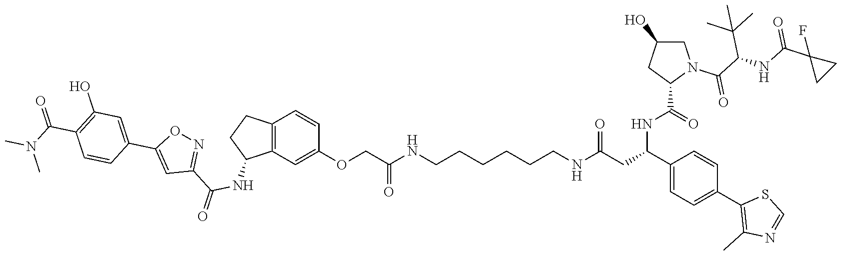 [SMILES: Cc1ncsc1-c1ccc([C@H](CC(=O)NCCCCCCNC(=O)COc2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-(2-((6- ((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5-yl)phe- nyl)propanamido)hexyl)amino)- 2-oxoethoxy)-2,3-dihydro-1H- inden-1-yl)isoxazole-3- carboxamide |
| 314 | LQ141- 37 | 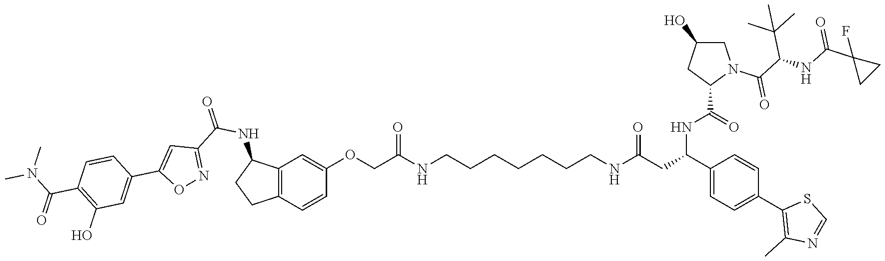 [SMILES: Cc1ncsc1-c1ccc([C@H](CC(=O)NCCCCCCCNC(=O)COc2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-(2-((7- ((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5-yl) phenyl)propanamido)heptyl)ami- no)-2-oxoethoxy)-2,3-dihydro-1H- inden-1-yl)isoxazole-3- carboxamide |
| 315 | LQ141- 38 | 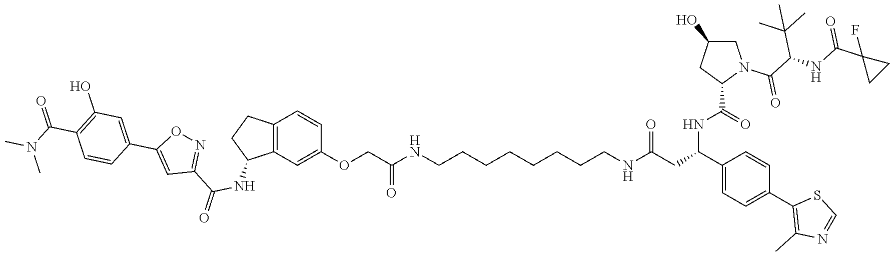 [SMILES: Cc1ncsc1-c1ccc([C@H](CC(=O)NCCCCCCCCNC(=O)COc2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-(2-((8- ((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5-yl)phe- nyl)propanamido)octyl)amino)- 2-oxoethoxy)-2,3-dihydro-1H- inden-1-yl)isoxazole-3- carboxamide |
| 316 | LQ141- 39 | 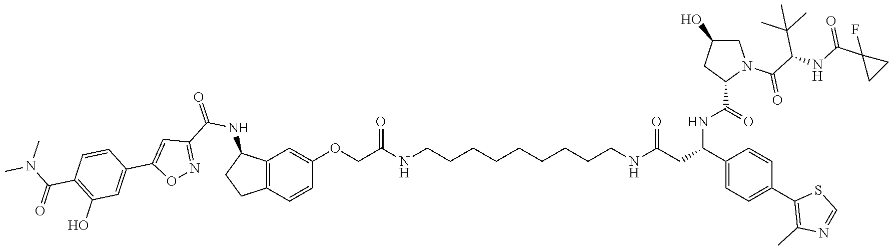 [SMILES: Cc1ncsc1-c1ccc([C@H](CC(=O)NCCCCCCCCCNC(=O)COc2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-(2-((9- ((S)-3-((2S,4R)-1-((S)-2-(1- fluorocyclopropane-1- carboxamido)-3,3- dimethylbutanoyl)-4- hydroxypyrrolidine-2- carboxamido)-3-(4-(4- methylthiazol-5-yl)phe- nyl)propanamido)nonyl)amino)- 2-oxoethoxy)-2,3-dihydro-1H- inden-1-yl)isoxazole-3- carboxamide |
| 317 | LQ141- 42 | 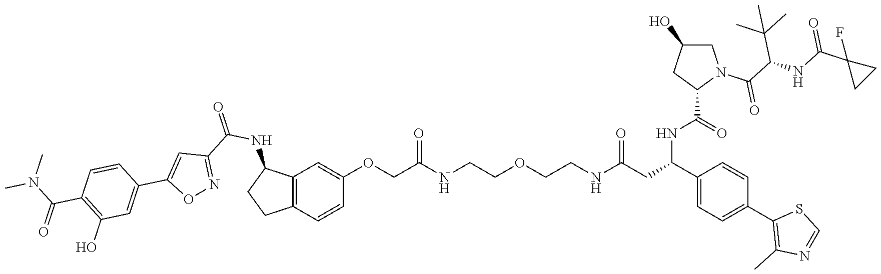 [SMILES: Cc1ncsc1-c1ccc([C@H](CC(=O)NCCOCCNC(=O)COc2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-(((S)-1- ((2S,4R)-1-((S)-2-(1- fluorocyclopropane-1- carboxamido)-3,3- dimethylbutanoyl)-4- hydroxypyrrolidin-2-yl)-3-(4-(4- methylthiazol-5-yl)phenyl)-1,5,13- trioxo-9-oxa-2,6,12- triazatetradecan-14-yl)oxy)-2,3- dihydro-1H-inden-1-yl)isoxazole- 3-carboxamide |
| 318 | LQ141- 43 | 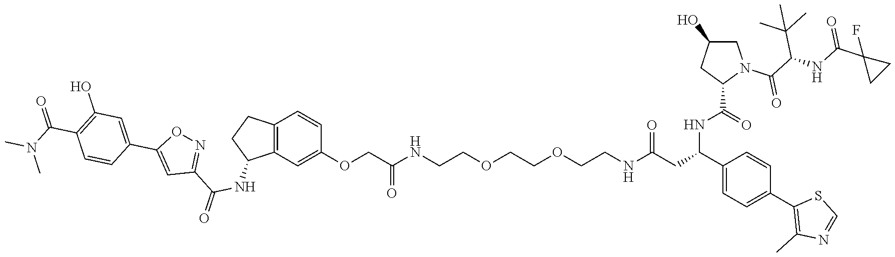 [SMILES: Cc1ncsc1-c1ccc([C@H](CC(=O)NCCOCCOCCNC(=O)COc2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-(((S)-1- ((2S,4R)-1-((S)-2-(1- fluorocyclopropane-1- carboxamido)-3,3- dimethylbutanoyl)-4- hydroxypryrrolidin-2-yl)-3-(4-(4- methylthiazol-5-yl)phenyl)-1,5,16- trioxo-9,12-dioxa-2,6,15- triazaheptadecan-17-yl)oxy)-2,3- dihydro-1H-inden-1-yl)isoxazole- 3-carboxamide |
| 319 | LQ141- 44 | 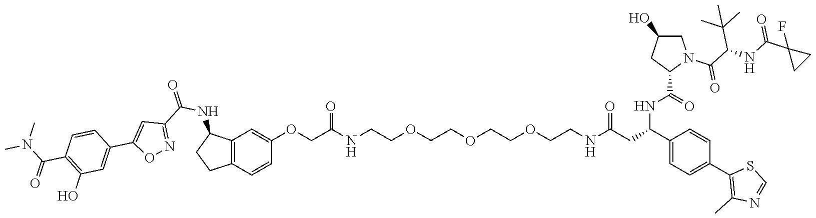 [SMILES: Cc1ncsc1-c1ccc([C@H](CC(=O)NCCOCCOCCOCCNC(=O)COc2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-(((S)-1- ((2S,4R)-1-((S)-2-(1- fluorocyclopropane-1- carboxamido)-3,3- dimethylbutanoyl)-4- hydroxypyrrolidin-2-yl)-3-(4-(4- methylthiazol-5-yl)phenyl)-1,5,19- trioxo-9,12,15-trioxa-2,6,18- triazaicosan-20-yl)oxy)-2,3- dihydro-1H-inden-1-yl)isoxazole- 3-carboxamide |
| 320 | LQ141- 45 | 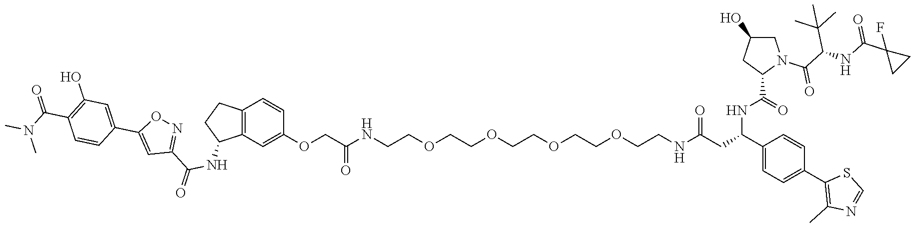 [SMILES: Cc1ncsc1-c1ccc([C@H](CC(=O)NCCOCCOCCOCCOCCNC(=O)COc2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-(((S)-1- ((2S,4R)-1-((S)-2-(1- fluorocyclopropane-1- carboxamido)-3,3- dimethylbutanoyl)-4- hydroxypyrrolidin-2-yl)-3-(4-(4- methylthiazol-5-yl)phenyl)-1,5,22- trioxo-9,12,15,18-tetraoxa-2,6,21- triazatricosan-23-yl)oxy)-2,3- dihydro-1H-inden-1-yl)isoxazole- 3-carboxamide |
| 321 | LQ141- 46 | 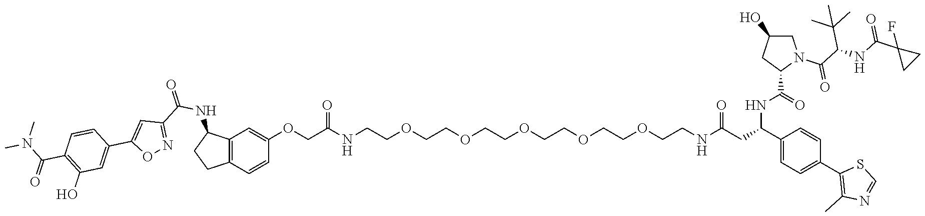 [SMILES: Cc1ncsc1-c1ccc([C@H](CC(=O)NCCOCCOCCOCCOCCOCCNC(=O)COc2ccc3c(c2)[C@H](NC(=O)c2cc(-c4ccc(C(=O)N(C)C)c(O)c4)on2)CC3)NC(=O)[C@@H]2C[C@@H](O)CN2C(=O)[C@@H](NC(=O)C2(F)CC2)C(C)(C)C)cc1] | 5-(4-(dimethylcarbamoyl)-3- hydroxyphenyl)-N-((R)-6-(((S)-1- ((2S,4R)-1-((S)-2-(1- fluorocyclopropane-1- carboxamido)-3,3- dimethylbutanoyl)-4- hydroxypyrrolidin-2-yl)-3-(4-(4- methylthiazol-5-yl)phenyl)-1,5,25- trioxo-9,12,15,18,21-pentaoxa- 2,6,24-triazahexacosan-26-yl)oxy)- 2,3-dihydro-1H-inden-1- yl)isoxazole-3-carboxamide |
| 322 | LQ141- 47 | 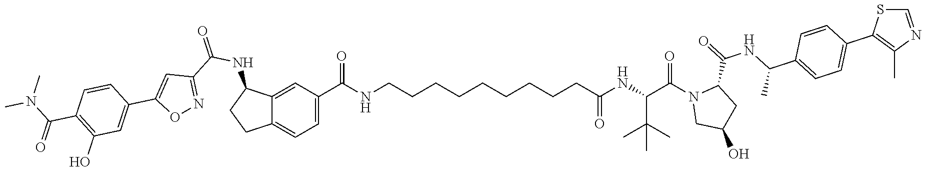 [SMILES: Cc1ncsc1-c1ccc([C@H](C)NC(=O)[C@@H]2C[C@@H](O)CN2C(=O)[C@@H](NC(=O)CCCCCCCCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3-hy- droxyphenyl)-N-((R)-6-((10-(((S)- 1-((2S,4R)-4-hydroxy-2-(((S)-1-(4- (4-methylthiazol-5-yl)phe- nyl)ethyl)carbamoyl)pyrrolidin- 1-yl)-3,3-dimethyl-1-oxobutan-2- yl)amino)-10-oxodecyl)carba- moyl)-2,3-dihydro-1H-inden-1- yl)isoxazole-3-carboxamide |
| 323 | LQ141- 48 | 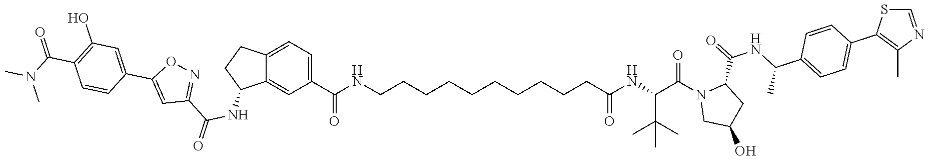 [SMILES: Cc1ncsc1-c1ccc([C@H](C)NC(=O)[C@@H]2C[C@@H](O)CN2C(=O)[C@@H](NC(=O)CCCCCCCCCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3- hydroxyphenyl)-N-((R)-6-((11-((S)- 1-((2S,4R)-4-hydroxy-2-(((S)-1-(4- (4-methylthiazol-5-yl)phe- nyl)ethyl)carbamoyl)pyrrolidin- 1-yl)-3,3-dimethyl-1-oxobutan-2- yl)amino)-11- oxoundecyl)carbamoyl)-2,3- dihydro-1H-inden-1-yl)isoxazole-3- carboxamide |
| 324 | LQ141- 49 | 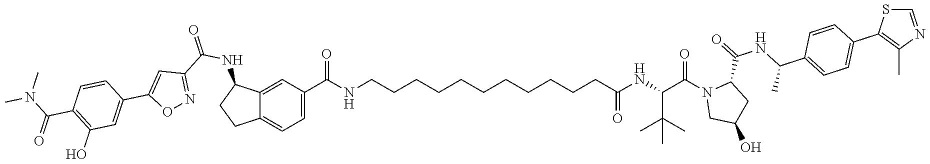 [SMILES: Cc1ncsc1-c1ccc([C@H](C)NC(=O)[C@@H]2C[C@@H](O)CN2C(=O)[C@@H](NC(=O)CCCCCCCCCCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3-hy- droxyphenyl)-N-((R)-6-((12-(((S)- 1-((2S,4R)-4-hydroxy-2-(((S)-1-(4- (4-methylthiazol-5-yl)phenyl)ethyl) carbamoyl)pyrrolidin-1-yl)-3,3- dimethyl-1-oxobutan-2-yl)amino)- 12-oxododecyl)carbamoyl)-2,3- dihydro-1H-inden-1-yl)isoxazole-3- carboxamide |
| 325 | LQ141- 52 | 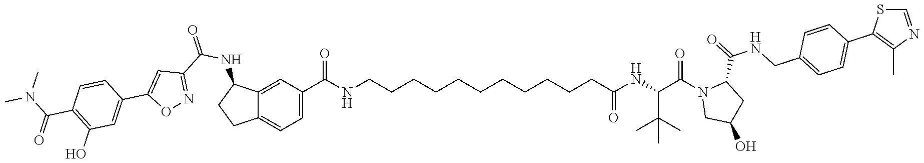 [SMILES: Cc1ncsc1-c1ccc(CNC(=O)[C@@H]2C[C@@H](O)CN2C(=O)[C@@H](NC(=O)CCCCCCCCCCCNC(=O)c2ccc3c(c2)[C@H](NC(=O)c2cc(-c4ccc(C(=O)N(C)C)c(O)c4)on2)CC3)C(C)(C)C)cc1] | 5-(4-(dimethylcarbamoyl)-3-hy- droxyphenyl)-N-((R)-6-((12-(((S)- 1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-12- oxododecyl)carbamoyl)-2,3- dihydro-1H-inden-1-yl)isoxazol-3- carboxamide |
| 326 | LQ141- 57 | 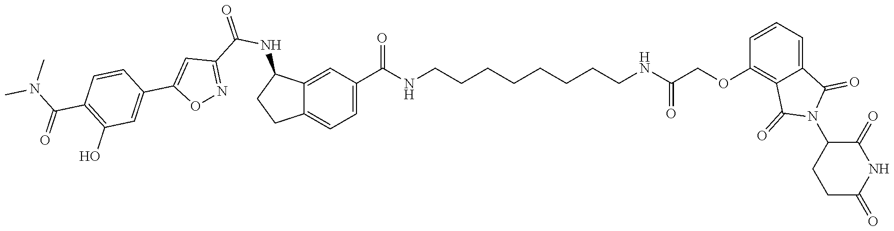 [SMILES: CN(C)C(=O)c1ccc(-c2cc(C(=O)N[C@@H]3CCc4ccc(C(=O)NCCCCCCCCNC(=O)COc5cccc6c5C(=O)N(C5CCC(=O)NC5=O)C6=O)cc43)no2)cc1O] | 5-(4-(dimethylcarbamoyl)-3- hydroxyphenyl)-N-((1R)-6-((8-(2- ((2-(2,6-dioxopiperidin-3-yl)-1,3- dioxoisoindolin-4-yl) oxy)acetamido)octyl)carbamoyl)- 2,3-dihydro-1H-inden-1- yl)isoxazole-3-carboxamide |

Compounds corresponding to Examples 1-326 have been synthesized and are provided with a Compound Code in Table 1.

As used herein, in case of discrepancy between the structure and chemical name provided for a particular compound, the given structure shall control.

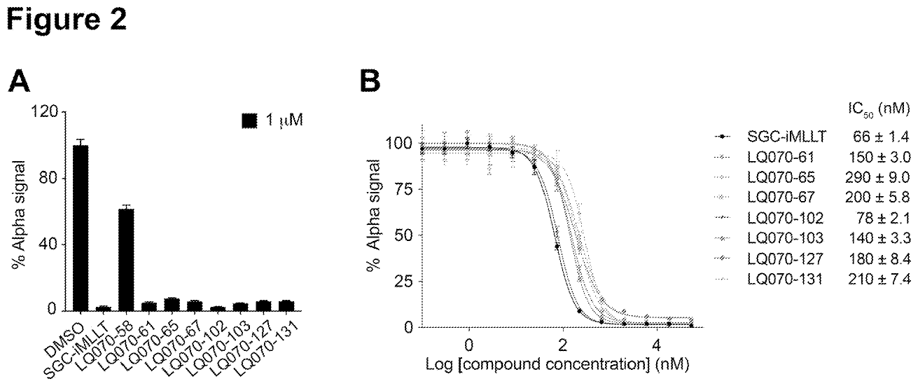

### Example 327. Precursors of ENL Degraders Show Strong Inhibition to the ENL YEATS Domain Binding to Acetylated Histone Peptide in AlphaScreen Assay (FIG. 2)

Inhibitory effect of precursors was tested at 1 μM in AlphaScreen assay (FIG. 2A), and IC₅₀ of these precursors except LQ070-58 was measured (FIG. 2B). Most of precursors maintained a good inhibitory effect compared with small molecule inhibitor SGC-iMLLT.

### Example 328. Effect of ENL Degraders on ENL-Dependent MV4; 11 Cell Growth (FIG. 3A-E)

ENL-dependent MV4; 11 cells were seeded at 2×10⁵ cells/mL density and treated with DMSO or the indicated compounds at 0.4, 2, 10 and 50 μM for 72 h. SGC-iMLLT was used as a control. Cell viability was measured using CellTiter-Glo reagent (Promega) and relative cell viability was calculated by normalization to DMSO samples.

### Example 329. Dose-Dependent Cell Growth Inhibition by Selected ENL Degraders (FIG. 4)

ENL-dependent MV4; 11 and ENL-independent Jurkat cells were seeded at 2×10⁵ cells/mL density and treated with DMSO or indicated compounds at 0.4, 2, 10 and 50 μM for 72 h. SGC-iMLLT was used as a control. Cell viability was measured using CellTiter-Glo reagent (Promega) and relative cell viability was calculated by normalization to DMSO samples.

### Example 330. ENL Degraders Induce ENL Protein Degradation (FIG. 5)

MV4; 11 cells were treated with DMSO or the indicated compounds (the same panel of ENL degraders as shown in FIG. 4) at 1 μM and 10 μM for 24 h. Cells were lysed and expression of ENL was assessed by Western blot analysis. Several compounds significantly reduced ENL protein levels.

### Example 331. ENL Degraders LQ076-122, LQ081-108 and LQ081-109 Concentration-Dependently Reduce ENL Protein Levels in MV4; 11 Cells (FIG. 6)

MV4; 11 cells were treated with LQ076-122, LQ081-108 or LQ081-109 at 0, 0.25, 0.5, 1, 1.5, 2, 2.5, 3, 4 and 8 μM for 24 h. Treatment with 8 μM of negative control compounds LQ081-107 (negative control of LQ076-122), LQ081-106 (negative control of LQ081-108), LQ081-158 (negative control of LQ081-109) or SGC-iMLLT were included as negative controls. The Western blot results show that LQ076-122, LQ081-108 and LQ081-109 reduced ENL protein levels in a concentration-dependent manner in MV4; 11 cells.

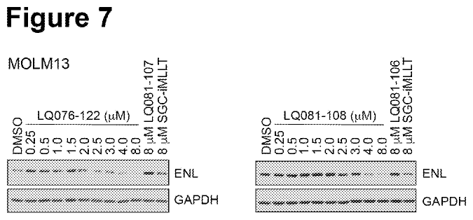

### Example 332. ENL Degraders LQ076-122 and LQ081-108 Concentration-Dependently Reduce ENL Levels in MOLM13 Cells (FIG. 7)

MOLM13 cells were treated with LQ076-122 or LQ081-108 at 0, 0.25, 0.5, 1, 1.5, 2, 2.5, 3, 4 and 8 μM for 24 h. Treatment with 8 μM of negative control compounds LQ081-107 (negative control of LQ076-122), LQ081-106 (negative control of LQ081-108), or SGC-iMLLT were included as negative controls. The Western blot results show that LQ076-122 and LQ081-108 reduced ENL protein levels in a concentration-dependent manner in MOLM13 cells.

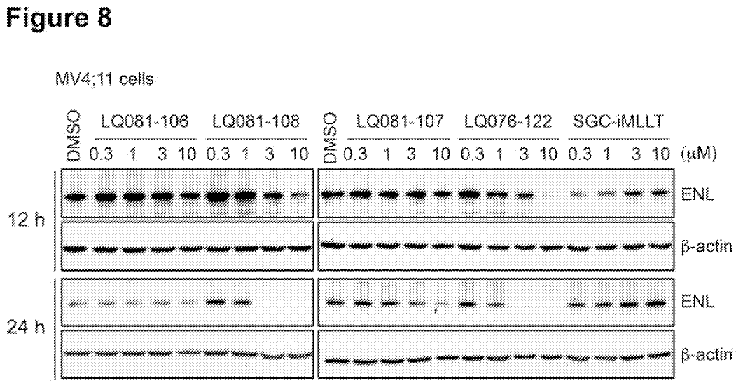

### Example 333. ENL Degraders LQ076-122 and LQ081-108 Reduce ENL Levels in a Concentration- and Time-Dependent Manner in MV4; 11 Cells (FIG. 8)

MV4; 11 cells were treated with LQ081-106, LQ081-108, LQ081-107, LQ076-122, or SGC-iMLLT at 0.3, 1, 3, and 10 μM for 12 and 24 h. DMSO treated cells were used as control. The Western blot results show that LQ076-122 and LQ081-108 reduced ENL protein levels in a concentration- and time-dependent manner in MV4; 11 cells. Negative control compounds and SGC-iMLLT did not affect ENL protein levels.

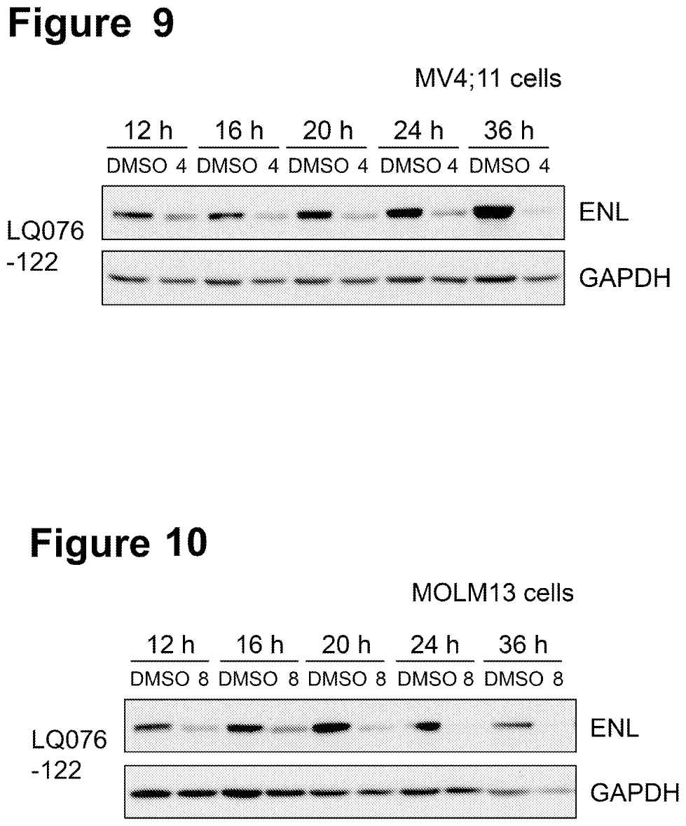

### Example 334. ENL Degrader LQ076-122 Time-Dependently Reduces ENL Protein Levels in MV4; 11 Cells at 4 μM Dose (FIG. 9)

MV4; 11 cells were treated with DMSO or 4 μM of LQ076-122 for 12, 16, 20, 24 and 36 h. The Western blot results show that LQ076-122 reduced ENL protein levels in a time-dependent manner.

### Example 335. ENL Degrader LQ076-122 Time-Dependently Reduces ENL Protein Levels in MOLM13 Cells at 8 μM Dose (FIG. 10)

MOLM13 cells were treated with DMSO or 8 μM of LQ076-122 for 12, 16, 20, 24 and 36 h. The Western blot results show that LQ076-122 reduced ENL protein levels in a time-dependent manner.

### Example 336. ENL Degrader LQ076-122 Selectively Reduces the Protein Levels of ENL, but not Another YEATS Domain-Containing Protein GAS41 (FIG. 11)

MV4; 11 cells were treated with LQ076-122, or LQ081-107 at 0.3, 1, 3, 10, and 30 μM for 24 h. DMSO treated cells were used as control. Cells were lysed and expression of ENL and GAS41 was assessed by Western analysis. The Western blot results show that LQ076-122 selectively reduced the ENL protein level, but not the level of another YEATS domain-containing protein GAS41.

### Example 337. Effect of Selected ENL Degraders on MV4; 11 Cell Growth (FIG. 12A-B)

MV4; 11 cells were seeded at 2×10⁵ cells/mL density and treated with DMSO or the indicated compounds at 0.5, 1, 2 and 4 μM for 72 h. SGC-iMLLT was used as a control. Cell viability was measured using CellTiter-Glo reagent (Promega) and relative cell viability was calculated by normalization to DMSO samples.

### Example 338. ENL Degraders LQ076-122, LQ081-108 and LQ081-109 Selectively Suppress Cell Growth of the ENL-Dependent MV4; 11 and MOLM13 Leukemia Cells, but not the ENL-Independent Jurkat Cells (FIG. 13A-C)

MV4; 11 (FIG. 13A), MOLM13 (FIG. 13B) and Jurkat (FIG. 13C) cells were seeded at 2×10⁵ cells/mL density and treated with DMSO or indicated compounds 0.5, 1, 2 and 4 μM for 72 h. Cell viability was measured using CellTiter-Glo reagent (Promega) and relative cell viability was calculated by normalization to DMSO samples. The results show that ENL degraders LQ076-122, LQ081-108 and LQ081-109 selectively suppressed cell growth of MV4; 11 and MOLM13 cells, but not Jurkat cells. SGC-iMLLT and the negative control compounds, including LQ108-4 (negative control of LQ076-122), LQ081-106 and LQ108-141 (negative controls of LQ081-108), and LQ081-158 and LQ108-142 (negative controls of LQ081-109), did not significantly affect cell growth of all three leukemia cell lines.

### Example 339. ENL Degraders LQ076-122 and LQ081-108 Concentration-Dependently Suppress ENL Target Gene Expression in MOLM13 Cells (FIG. 14A-B)

MOLM13 cells were treated with LQ076-122 (FIG. 14A) and LQ081-108 (FIG. 14B) at 0.5, 1, 2, 4, and 8 μM for 24 h. Treatment with DMSO, 8 μM of SGC-iMLLT or LQ081-107 (negative control of LQ076-122, FIG. 14A) and LQ081-106 (negative control of LQ081-108) were included for comparison. RT-qPCR analysis was performed to detect the mRNA levels of selected ENL target genes. The results show that LQ076-122 and LQ081-108 reduced ENL target gene expression in a concentration-dependent manner, whereas SGC-iMLLT and negative control compounds did not dramatically affect these genes.

### Example 340. ENL Degrader LQ076-122 Suppresses ENL Target Gene Expression in a Concentration- and Time-Dependent Manner in MV4; 11 Cells (FIG. 15)

MV4; 11 cells were treated with DMSO, or LQ076-122 at 1, 2, and 4 μM for 6, 12, 18 and 24 h. RT-qPCR analysis was performed to detect the mRNA levels of selected ENL target genes. Results showed that LQ076-122 reduced ENL target gene expression in a concentration- and time-dependent manner.

### Example 341. ENL Degrader LQ076-122 Induces Apoptosis in MV4; 11 and MOLM13 Cells (FIG. 16A-B)

MV4; 11 (FIG. 16A) and MOLM13 (FIG. 16B) cells were treated with DMSO, or LQ076-122, LQ108-4 (negative control of LQ076-122) and SGC-iMLLT at 1, 2, and 4 μM for 24 h. Apoptotic cells were measured by the FITC Annexin V Apoptosis Detection Kit (BD Biosciences). The results show that the ENL degrader LQ076-122, but not the negative control compound LQ108-4 or SGC-iMLLT, induced apoptosis.

### Example 342. Plasma Concentration of ENL Degrader LQ076-122 Over 12 h Following a Single 50 mg/kg IP Injection in Mice (FIG. 17)

Three C57BL/6 mice at 6-8 weeks of age were used in PK study for each time point. After a single dose intraperitoneal (IP) injection of ENL degrader LQ076-122 (50 mg/kg), plasma concentrations of degrader were measured at 6 time points (0.5, 1, 2, 4, 8 and 12 h) from each test animal. The concentrations of LQ076-122 in plasma were maintained above 2 μM for 6 h with the maximum plasma concentration of about 6 μM.

### Example 343. ENL Degrader LQ076-122 Significantly Delays the Leukemia Progression in an MV4; 11 Disseminated Xenograft Model (FIG. 18A-B)

Immuno-deficient NSG mice were irradiated and transplanted with 5×10⁵ MV4; 11-Luc cells through tail-vein injections. Ten days after transplantation, mice (n=5) were treated with 100 mg/kg LQ076-122 or vehicle twice daily through IP injection in cycles. Each cycle contains 4 treatment days followed by 2 resting days. Day 0 is the time that the treatment started. Leukemia progression was monitored by bioluminescence imaging at different time points upon LQ076-122 or vehicle treatment (FIG. 18A). The mean radiances of bioluminescence signal were quantified in FIG. 18B.

### Example 344. ENL Degraders Induce ENL Protein Degradation (FIG. 19A-D)

MV4; 11 cells stably expressing 3Flag-HA-tagged ENL were treated with DMSO or the indicated compounds at 1 μM and 10 μM for 24 h. Cells were lysed and expression of 3Flag-HA-ENL was assessed by Western blot analysis. A panel of compounds significantly reduced ENL protein levels.

### Example 345. ENL Degraders Induce ENL Protein Degradation (FIG. 20A-B)

MV4; 11 cells stably expressing 3Flag-HA-tagged ENL were treated with DMSO or the indicated compounds at 1 μM and 10 μM for 6 h. Cells were lysed and expression of 3Flag-HA-ENL was assessed by Western blot analysis. A panel of compounds significantly reduced ENL protein levels.

### Example 346. ENL Degraders Induce ENL Protein Degradation (FIG. 21)

MV4; 11 cells were treated with DMSO or the indicated compounds at 1 μM and 10 μM for 6 h. Cells were lysed and expression of endogenous ENL was assessed by Western blot analysis. Several compounds significantly reduced ENL protein levels.

### Example 347. ENL Degraders LQ108-69, LQ108-71, LQ108-72, LQ126-62 and LQ126-63 Concentration-Dependently Reduce ENL Levels in Cells (FIG. 22)

MV4; 11, MOLM13 and Jurkat cells were treated with LQ108-69, LQ108-71, LQ108-72, LQ126-62 and LQ126-63 at 0, 1 nM, 10 nM, 100 nM, 1 μM, and 10 μM doses for 6 h. DMSO was used as negative control. The Western blot results show that LQ108-69, LQ108-71, LQ108-72, LQ126-62 and LQ126-63 reduced ENL protein levels in a concentration-dependent manner in all three tested cell lines.

### Example 348. ENL Degraders LQ108-69, LQ108-70, LQ108-71, LQ108-72, LQ126-62 and LQ126-63 Maintain the ENL Protein at Low Levels after 48 and 72 h Treatment (FIG. 23)

MV4; 11, MOLM13 and Jurkat cells were treated with LQ108-69, LQ108-70, LQ108-71, LQ108-72, LQ126-62 and LQ126-63 at 1 μM for 48 and 72 h. DMSO treated cells were used as control. The Western blot results show that LQ108-69, LQ108-70, LQ108-71, LQ108-72, LQ126-62 and LQ126-63 maintained the ENL protein at low levels after 48 and 72 h treatment.

### Example 349. ENL Degrader LQ108-63, LQ108-69, LQ108-70, LQ126-62 and LQ126-63 Reduce ENL Protein Level Through Proteasome-Mediated Degradation (FIG. 24)

MG132 treatment partially blocks the ENL degradation induced by degraders LQ108-63, LQ108-69, LQ108-70, LQ126-62 and LQ126-63 in MV4; 11 cells. Cells were treated with 1 μM of ENL degrader with or without 1 μM proteasome inhibitor MG132 for 6 h.

### Example 350. Effect of ENL Degraders on ENL-Dependent MV4; 11 Cell Growth (FIG. 25)

ENL-dependent MV4; 11 cells were seeded at 2×10⁵ cells/mL density and treated with DMSO or the indicated compounds at 0, 1.25, 2.5, 5 and 10 μM for 72 h. Cell viability was measured using CellTiter-Glo reagent (Promega) and relative cell viability was calculated by normalization to DMSO samples.

### Example 351. Dose-Dependent Cell Growth Inhibition by ENL Degrader LQ126-63 (FIG. 26)

ENL-dependent MV4; 11 and ENL-independent Jurkat cells were seeded at 2×10⁵ cells/mL density and treated with DMSO or indicated compounds at 10 nM, 100 nM, 1 μM and 10 μM for 3 days (A) or 6 days (B). Cell viability was measured using CellTiter-Glo reagent (Promega) and relative cell viability was calculated by normalization to DMSO samples.

Materials And Methods:

General Chemistry Methods

For the synthesis of intermediates and examples, HPLC spectra for all compounds were acquired using an Agilent 1200 Series system with DAD detector. Chromatography was performed on a 2.1×150 mm Zorbax 300SB-C18 5 μm column with water containing 0.1% formic acid as solvent A and acetonitrile containing 0.1% formic acid as solvent B at a flow rate of 0.4 ml/min. The gradient program was as follows: 1% B (0-1 min), 1-99% B (1-4 min), and 99% B (4-8 min). High-resolution mass spectra (HRMS) data were acquired in positive ion mode using an Agilent G1969A API-TOF with an electrospray ionization (ESI) source. Nuclear Magnetic Resonance (NMR) spectra were acquired on a Bruker DRX-600 spectrometer with 600 MHz for proton (¹H NMR) and 150 MHz for carbon (¹³C NMR); chemical shifts are reported in (6). Preparative HPLC was performed on Agilent Prep 1200 series with UV detector set to 254 nm. Samples were injected onto a Phenomenex Luna 250×30 mm, 5 μm, C₁₈ column at room temperature. The flow rate was 40 ml/min. A linear gradient was used with 10% (or 50%) of MeOH (A) in H₂O (with 0.1% TFA) (B) to 100% of MeOH (A). HPLC was used to establish the purity of target compounds. All final compounds had >95% purity using the HPLC methods described above.

AlphaScreen Assay

IC₅₀ of ENL degrader precursor in inhibition of ENL YEATS-H3K9ac interaction was measured by AlphaScreen assay using AlphaScreen Histidine (Nickel Chelate) Detection Kit (PerkinElmer). Assays were set up in 30 μL volume with 100 nM His tagged-ENL YEATS protein, 30 nM biotinylated-H3K9ac peptide, indicated concentrations of ENL degrader precursor, 10 μg/mL of streptavidin-coated donor beads and 10 μg/mL of chelate nickle-coated acceptor beads in Alpha assay buffer (50 mM HEPES pH 7.4, 100 mM NaCl, 1.0 mg/mL BSA, and 0.05% CHAPS). Alpha signals were detected by an EnVision microplate reader equipped with an Alpha laser (PerkinElmer).

Cell Lines

All cell lines were purchased from ATCC. MV4; 11, MOLM13, and Jurkat were cultured in RPMI1640 supplemented with 10% FBS and 1% Penicillin/Streptomycin.

Compound Treatment

ENL degraders were dissolved in DMSO. DMSO with no degraders was used as the control. 1×10⁶ leukemia cells were seeded in 5 mL medium. For prescreening of compounds, each test compound was added to the medium at 1 μM and 10 μM. Cells were collected after 24 h treatment. For the concentration-dependent treatment, candidate compounds were added to the medium at a series of concentration as indicated in figures. Cells were collected after 24 h treatment. For the time-course treatment, candidate compounds were added to the medium at a final concentration of 4 μM (MV4; 11 cells) or 8 μM (MOLM13 cells). Cells were collected at the indicated timepoints (in hours: 12, 16, 20, 24 and 36 h).

Immunoblotting

After ENL degrader treatment, cells were collected, lysed, and total cell lysates were used for Western blot. The following primary antibodies were used: ENL (Cell Signaling Technology), GAS41 (Santa Cruz), GAPDH (Santa Cruz), β-actin (Sigma). Blots were detected using HRP-conjugated secondary antibodies.

Cell Viability Assay

MV4; 11 or MOLM13 cells were seeded at 0.2×10⁶ cells/mL density. Cells were treated with DMSO or ENL degraders at indicated concentrations. Each treatment was done in triplicates. After 72 h treatment, 100 μL of cell suspension from each treatment was mixed with 25 μL of CellTiter-Glo reagent (Promega) and incubated for 10 min before the luminescence signals were detected on a plate reader.

Apoptosis Assay

MV4; 11 or MOLM13 cells were seeded at 0.2×10⁶ cells/mL density. Cells were treated with DMSO or ENL degraders at indicated concentrations. Each treatment was done in triplicates. After 24 h treatment, cells were collected and washed with ice-cold PBS once and resuspended in 250 μL of 1× binding buffer containing 5 μL of FITC-Annexin V and PI (BD Biosciences). After 15 min incubation at room temperature in the dark, 250 μL of 1× binding buffer was added and flow cytometry analysis was performed.

RNA Extraction and RT-qPCR

Total RNA was extracted using the RNeasy Plus kit (Qiagen) and reverse-transcribed using the iScript cDNA Synthesis kit (Bio-Rad). RT-qPCR was performed using the Power SYBR Green PCR Master Mix (Applied Biosystems) on the CFX96 Real-Time PCR system (Bio-Rad). Gene expressions were calculated following normalization to 18s rRNA amounts using the comparative cycle threshold (Ct) method.

In Vivo Pharmacokinetics (PK) Study

The standard mouse PK study was conducted by Charles River Laboratories. Three C57BL/6 mice at 6-8 weeks of age were used for each time point. After a single dose intraperitoneal (IP) injection of ENL degrader (50 mg/kg), plasma concentrations of degrader were measured at 4 time points (in hours: 0.5, 1, 2, 4, 8 and 12 h) from each test animal.

Tumor Xenograft Study

Immunodeficient NOD.Cg-Prkdc^{scid}Il2rg^{tm1wil}/SzJ (NSG) mice at 6-8 weeks of age were produced at the Van Andel Institute Vivarium and Transgenic Core using breeders purchased from the Jackson laboratory. Mice were pretreated with acidified water and antibiotics for a week before a sublethal dose of total body irradiated (2 Gy). Then mice were transplanted with 0.5×10⁶ MV4; 11-Luc cells through tail-vein injection. ENL degrader treatment was started ten days after transplantation with the successful engraftment confirmed by bioluminescence imaging. Mice were randomly assigned to two groups (n=5) and treated with IP injections of either ENL degrader LQ076-122 (100 mg/kg, twice daily) or vehicle. The treatment lasted for 4 consecutive days followed by a 2-day rest, and was repeated in three cycles. Leukemia progression in each animal was monitored by bioluminescence imaging after each treatment cycle. For whole-body bioluminescent imaging, mice were IP injected with 150 mg/kg D-luciferin 10 min prior to imaging using an AMI-1000 imaging system (Spectral Instruments Imaging). Mice were euthanized when they reached moribund stage according to the approved IACUC protocol. All procedures and studies with mice were performed in accordance with protocols approved by the Institutional Animal Care and Use Committee of the Van Andel Institute.

### Other Aspects

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Abramovich, C., and Humphries, R. K. (2005). Hox regulation of normal and leukemic hematopoietic stem cells. Curr Opin Hematol 12, 210-216.
Armstrong, S. A., Staunton, J. E., Silverman, L. B., Pieters, R., de Boer, M. L., Minden, M. D., Sallan, S. E., Lander, E. S., Golub, T. R., and Korsmeyer, S. J. (2002). MLL translocations specify a distinct gene expression profile that distinguishes a unique leukemia. Nat Genet 30, 41-47. Artinger, E. L., Mishra, B. P., Zaffuto, K. M., Li, B. E., Chung, E. K. Y., Moore, A. W., Chen, Y. F., Cheng, C., and Ernst, P. (2013). An MLL-dependent network sustains hematopoiesis. P Natl Acad Sci USA 110, 12000-12005.
Asiaban, J. N., Milosevich, N., Chen, E., Bishop, T. R., Wang, J., Zhang, Y., Ackerman, C. J., Hampton, E. N., Young, T. S., Hull, M. V., et al. (2020). Cell-Based Ligand Discovery for the ENL YEATS Domain. ACS Chem Biol 15, 895-903.
Ayton, P. M., and Cleary, M. L. (2001). Molecular mechanisms of leukemogenesis mediated by MLL fusion proteins. Oncogene 20, 5695-5707.
Biondi, A., Cimino, G., Pieters, R., and Pui, C. H. (2000). Biological and therapeutic aspects of infant leukemia. Blood 96, 24-33.
Biswas, D., Milne, T. A., Basrur, V., Kim, J., Elenitoba-Johnson, K. S., Allis, C. D., and Roeder, R. G. (2011). Function of leukemogenic mixed lineage leukemia 1 (MLL) fusion proteins through distinct partner protein complexes. Proc Natl Acad Sci USA 108, 15751-15756.
Bitoun, E., Oliver, P. L., and Davies, K. E. (2007). The mixed-lineage leukemia fusion partner AF4 stimulates RNA polymerase II transcriptional elongation and mediates coordinated chromatin remodeling. Human molecular genetics 16, 92-106.
Bondeson, D. P., Mares, A., Smith, I. E., Ko, E., Campos, S., Miah, A. H., Mulholland, K. E., Routly, N., Buckley, D. L., Gustafson, J. L., et al. (2015). Catalytic in vivo protein knockdown by small-molecule PROTACs. Nat Chem Biol 11, 611-617.
Buckley, D. L., and Crews, C. M. (2014). Small-molecule control of intracellular protein levels through modulation of the ubiquitin proteasome system. Angew Chem Int Ed Engl 53, 2312-2330.
Buckley, D. L., Gustafson, J. L., Van Molle, I., Roth, A. G., Tae, H. S., Gareiss, P. C., Jorgensen, W. L., Ciulli, A., and Crews, C. M. (2012a). Small-molecule inhibitors of the interaction between the E3 ligase VHL and HIF1alpha. Angew Chem Int Ed Engl 51, 11463-11467.
Buckley, D. L., Raina, K., Darricarrere, N., Hines, J., Gustafson, J. L., Smith, I. E., Miah, A. H., Harling, J. D., and Crews, C. M. (2015). HaloPROTACS: Use of Small Molecule PROTACs to Induce Degradation of HaloTag Fusion Proteins. ACS Chem Biol 10, 1831-1837.
Buckley, D. L., Van Molle, I., Gareiss, P. C., Tae, H. S., Michel, J., Noblin, D. J., Jorgensen, W. L., Ciulli, A., and Crews, C. M. (2012b). Targeting the von Hippel-Lindau E3 ubiquitin ligase using small molecules to disrupt the VHL/HIF-1alpha interaction. J Am Chem Soc 134, 4465-4468.
Chamberlain, P. P., Lopez-Girona, A., Miller, K., Carmel, G., Pagarigan, B., Chie-Leon, B., Rychak, E., Corral, L. G., Ren, Y. J., Wang, M., et al. (2014). Structure of the human Cereblon-DDB1-lenalidomide complex reveals basis for responsiveness to thalidomide analogs. Nat Struct Mol Biol 21, 803-809.
Christott, T., Bennett, J., Coxon, C., Monteiro, O., Giroud, C., Beke, V., Felce, S. L., Gamble, V., Gileadi, C., Poda, G., et al. (2019). Discovery of a Selective Inhibitor for the YEATS Domains of ENL/AF9. SLAS Discov 24, 133-141.
Deshpande, A. J., Bradner, J., and Armstrong, S. A. (2012). Chromatin modifications as therapeutic targets in MLL-rearranged leukemia. Trends Immunol 33, 563-570.
Erb, M. A., Scott, T. G., Li, B. E., Xie, H., Paulk, J., Seo, H. S., Souza, A., Roberts, J. M., Dastjerdi, S., Buckley, D. L., et al. (2017). Transcription control by the ENL YEATS domain in acute leukaemia. Nature 543, 270-274.
Ferrando, A. A., Armstrong, S. A., Neuberg, D. S., Sallan, S. E., Silverman, L. B., Korsmeyer, S. J., and Look, A. T. (2002). Gene expression signatures in MLL-rearranged B-precursor and T lineage acute leukemias: Dominance of HOX dysregulation. Blood 100, 310A-310A.
Fischer, E. S., Bohm, K., Lydeard, J. R., Yang, H., Stadler, M. B., Cavadini, S., Nagel, J., Serluca, F., Acker, V., Lingaraju, G. M., et al. (2014). Structure of the DDB1-CRBN E3 ubiquitin ligase in complex with thalidomide. Nature 512, 49-53.
Gadd, S., Huff, V., Walz, A. L., Ooms, A., Armstrong, A. E., Gerhard, D. S., Smith, M. A., Auvil, J. M. G., Meerzaman, D., Chen, Q. R., et al. (2017). A Children's Oncology Group and TARGET initiative exploring the genetic landscape of Wilms tumor. Nat Genet 49, 1487-1494.
Galdeano, C., Gadd, M. S., Soares, P., Scaffidi, S., Van Molle, I., Birced, I., Hewitt, S., Dias, D. M., and Ciulli, A. (2014). Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities. J Med Chem 57, 8657-8663.
He, N., Chan, C. K., Sobhian, B., Chou, S., Xue, Y., Liu, M., Alber, T., Benkirane, M., and Zhou, Q. (2011). Human Polymerase-Associated Factor complex (PAFc) connects the Super Elongation Complex (SEC) to RNA polymerase II on chromatin. Proc Natl Acad Sci USA 108, E636-645.
He, N. H., Liu, M., Hsu, J., Xue, Y. H., Chou, S., Burlingame, A., Krogan, N. J., Alber, T., and Zhou, Q. (2010). HIV-1 Tat and Host AFF4 Recruit Two Transcription Elongation Factors into a Bifunctional Complex for Coordinated Activation of HIV-1 Transcription. Mol Cell 38, 428-438.
Heidenreich, D., Moustakim, M., Schmidt, J., Merk, D., Brennan, P. E., Fedorov, O., Chaikuad, A., and Knapp, S. (2018). Structure-Based Approach toward Identification of Inhibitory Fragments for Eleven-Nineteen-Leukemia Protein (ENL). J Med Chem 61, 10929-10934.
Hess, J. L. (2004). MLL: a histone methyltransferase disrupted in leukemia. Trends Mol Med 10, 500-507.
Hsu, C. C., Shi, J., Yuan, C., Zhao, D., Jiang, S., Lyu, J., Wang, X., Li, H., Wen, H., Li, W., et al. (2018). Recognition of histone acetylation by the GAS41 YEATS domain promotes H2A.Z deposition in non-small cell lung cancer. Genes & development 32, 58-69.
Ito, T., Ando, H., Suzuki, T., Ogura, T., Hotta, K., Imamura, Y., Yamaguchi, Y., and Handa, H. (2010). Identification of a primary target of thalidomide teratogenicity. Science 327, 1345-1350.
Jude, C. D., Climer, L., Xu, D., Artinger, E., Fisher, J. K., and Ernst, P. (2007). Unique and independent roles for MLL in adult hematopoietic stem cells and progenitors. Cell Stem Cell 1, 324-337.
Klein, B. J., Ahmad, S., Vann, K. R., Andrews, F. H., Mayo, Z. A., Bourriquen, G., Bridgers, J. B., Zhang, J., Strahl, B. D., Cote, J., et al. (2018). Yaf9 subunit of the NuA4 and SWR1 complexes targets histone H3K27ac through its YEATS domain. Nucleic Acids Res 46, 421-430.
Krivtsov, A. V., and Armstrong, S. A. (2007). MLL translocations, histone modifications and leukaemia stem-cell development. Nature reviews Cancer 7, 823-833.
Lai, A. C., Toure, M., Hellerschmied, D., Salami, J., Jaime-Figueroa, S., Ko, E., Hines, J., and Crews, C. M. (2016). Modular PROTAC Design for the Degradation of Oncogenic BCR-ABL. Angew Chem Int Ed Engl 55, 807-810.
Li, X., Li, X. M., Jiang, Y., Liu, Z., Cui, Y., Fung, K. Y., van der Beelen, S. H. E., Tian, G., Wan, L., Shi, X., et al. (2018). Structure-guided development of YEATS domain inhibitors by targeting pi-pi-pi stacking. Nature chemical biology 14, 1140-1149.
Li, Y., Sabari, B. R., Panchenko, T., Wen, H., Zhao, D., Guan, H., Wan, L., Huang, H., Tang, Z., Zhao, Y., et al. (2016). Molecular Coupling of Histone Crotonylation and Active Transcription by AF9 YEATS Domain. Mol Cell 62, 181-193.
Li, Y., Wen, H., Xi, Y., Tanaka, K., Wang, H., Peng, D., Ren, Y., Jin, Q., Dent, S. Y., Li, W., et al. (2014). AF9 YEATS domain links histone acetylation to DOTiL-mediated H₃K79 methylation. Cell 159, 558-571.
Lin, C., Smith, E. R., Takahashi, H., Lai, K. C., Martin-Brown, S., Florens, L., Washburn, M. P., Conaway, J. W., Conaway, R. C., and Shilatifard, A. (2010). AFF4, a component of the ELL/P-TEFb elongation complex and a shared subunit of MLL chimeras, can link transcription elongation to leukemia. Mol Cell 37, 429-437.
Lu, J., Qian, Y., Altieri, M., Dong, H., Wang, J., Raina, K., Hines, J., Winkler, J. D., Crew, A. P., Coleman, K., et al. (2015). Hijacking the E3 Ubiquitin Ligase Cereblon to Efficiently Target BRD4. Chemistry & biology 22, 755-763.
Marschalek, R. (2015). MLL Leukemia and Future Treatment Strategies. Archiv der Pharmazie 348, 221-228.
Meyer, C., Hofmann, J., Burmeister, T., Groger, D., Park, T. S., Emerenciano, M., de Oliveira, M. P., Renneville, A., Villarese, P., Macintyre, E., et al. (2013). The MLL recombinome of acute leukemias in 2013. Leukemia 27, 2165-2176.
Meyer, C., Kowarz, E., Hofmann, J., Renneville, A., Zuna, J., Trka, J., Ben Abdelali, R., Macintyre, E., De Braekeleer, E., De Braekeleer, M., et al. (2009). New insights to the MLL recombinome of acute leukemias. Leukemia 23, 1490-1499.
Meyer, C., Schneider, B., Jakob, S., Strehl, S., Attarbaschi, A., Schnittger, S., Schoch, C., Jansen, M. W. J. C., Dongen, J. J. M., Boer, M. L., et al. (2006). The MLL recombinome of acute leukemias. Leukemia 20, 777-784.
Mi, W., Guan, H., Lyu, J., Zhao, D., Xi, Y., Jiang, S., Andrews, F. H., Wang, X., Gagea, M., Wen, H., et al. (2017). YEATS2 links histone acetylation to tumorigenesis of non-small cell lung cancer. Nat Commun 8, 1088.
Mohan, M., Herz, H. M., Takahashi, Y. H., Lin, C., Lai, K. C., Zhang, Y., Washburn, M. P., Florens, L., and Shilatifard, A. (2010a). Linking H3K79 trimethylation to Wnt signaling through a novel Dot1-containing complex (DotCom). Genes & development 24, 574-589.
Mohan, M., Lin, C., Guest, E., and Shilatifard, A. (2010b). Licensed to elongate: a molecular mechanism for MLL-based leukaemogenesis. Nature reviews Cancer 10, 721-728.
Moustakim, M., Christott, T., Monteiro, O. P., Bennett, J., Giroud, C., Ward, J., Rogers, C. M., Smith, P., Panagakou, I., Diaz-Saez, L., et al. (2018a). Discovery of an MLLT1/3 YEATS Domain Chemical Probe. Angew Chem Int Ed Engl 57, 16302-16307.
Mueller, D., Bach, C., Zeisig, D., Garcia-Cuellar, M. P., Monroe, S., Sreekumar, A., Zhou, R., Nesvizhskii, A., Chinnaiyan, A., Hess, J. L., et al. (2007). A role for the MLL fusion partner ENL in transcriptional elongation and chromatin modification. Blood 110, 4445-4454.
Mueller, D., Garcia-Cuellar, M. P., Bach, C., Buhl, S., Maethner, E., and Slany, R. K. (2009). Misguided Transcriptional Elongation Causes Mixed Lineage Leukemia. Plos Biol 7. Ni, X., Heidenreich, D., Christott, T., Bennett, J., Moustakim, M., Brennan, P. E., Fedorov, O., Knapp, S., and Chaikuad, A. (2019). Structural Insights into Interaction Mechanisms of Alternative Piperazine-urea YEATS Domain Binders in MLLT1. ACS Med Chem Lett 10, 1661-1666.
Okada, Y., Feng, Q., Lin, Y., Jiang, Q., Li, Y., Coffield, V. M., Su, L., Xu, G., and Zhang, Y. (2005). hDOT1L links histone methylation to leukemogenesis. Cell 121, 167-178.
Perlman, E. J., Gadd, S., Arold, S. T., Radhakrishnan, A., Gerhard, D. S., Jennings, L., Huff, V., Guidry Auvil, J. M., Davidsen, T. M., Dome, J. S., et al. (2015). MLLT1 YEATS domain mutations in clinically distinctive Favourable Histology Wilms tumours. Nat Commun 6, 10013.
Pieters, R., Schrappe, M., De Lorenzo, P., Hann, I., De Rossi, G., Felice, M., Hovi, L., LeBlanc, T., Szczepanski, T., Ferster, A., et al. (2007). A treatment protocol for infants younger than 1 year with acute lymphoblastic leukaemia (Interfant-99): an observational study and a multicentre randomised trial. Lancet 370, 240-250.
Pui, C. H., Campana, D., Pei, D. Q., Bowman, W. P., Sandlund, J. T., Kaste, S. C., Ribeiro, R. C., Rubnitz, J. E., Raimondi, S. C., Onciu, M., et al. (2009). Treating Childhood Acute Lymphoblastic Leukemia without Cranial Irradiation. New Engl J Med 360, 2730-2741.
Rao, R. C., and Dou, Y. (2015). Hijacked in cancer: the KMT2 (MLL) family of methyltransferases. Nature reviews Cancer 15, 334-346.
Shanle, E. K., Andrews, F. H., Meriesh, H., McDaniel, S. L., Dronamraju, R., DiFiore, J. V., Jha, D., Wozniak, G. G., Bridgers, J. B., Kerschner, J. L., et al. (2015). Association of Taf14 with acetylated histone H3 directs gene transcription and the DNA damage response. Genes & development 29, 1795-1800.
Slany, R. K. (2005). When epigenetics kills: MLL fusion proteins in leukemia. Hematol Oncol 23, 1-9.
Wan, L., Chong, S., Xuan, F., Liang, A., Cui, X., Gates, L., Carroll, T. S., Li, Y., Feng, L., Chen, G., et al. (2020). Impaired cell fate through gain-of-function mutations in a chromatin reader. Nature 577, 121-126.
Wan, L., Wen, H., Li, Y., Lyu, J., Xi, Y., Hoshii, T., Joseph, J. K., Wang, X., Loh, Y. E., Erb, M. A., et al. (2017). ENL links histone acetylation to oncogenic gene expression in acute myeloid leukaemia. Nature 543, 265-269.
Winter, G. E., Buckley, D. L., Paulk, J., Roberts, J. M., Souza, A., Dhe-Paganon, S., and Bradner, J. E. (2015). Phthalimide conjugation as a strategy for in vivo target protein degradation. Science 348, 1376-1381.
Xie, T., Lim, S. M., Westover, K. D., Dodge, M. E., Ercan, D., Ficarro, S. B., Udayakumar, D., Gurbani, D., Tae, H. S., Riddle, S. M., et al. (2014). Pharmacological targeting of the pseudokinase Her3. Nat Chem Biol 10, 1006-1012.
Yokoyama, A., Lin, M., Naresh, A., Kitabayashi, I., and Cleary, M. L. (2010). A Higher-Order Complex Containing AF4 and ENL Family Proteins with P-TEFb Facilitates Oncogenic and Physiologic MLL-Dependent Transcription. Cancer Cell 17, 198-212.
Yu, B. D., Hess, J. L., Horning, S. E., Brown, G. A. J., and Korsmeyer, S. J. (1995). Altered Hox Expression and Segmental Identity in Mll-Mutant Mice. Nature 378, 505-508.
Zengerle, M., Chan, K. H., and Ciulli, A. (2015). Selective Small Molecule Induced Degradation of the BET Bromodomain Protein BRD4. ACS Chem Biol 10, 1770-1777. Zhang, Q., Zeng, L., Zhao, C., Ju, Y., Konuma, T., and Zhou, M. M. (2016). Structural Insights into Histone Crotonyl-Lysine Recognition by the AF9 YEATS Domain. Structure 24, 1606-1612.

1. A bivalent compound comprising a degrader/disruption tag EL conjugated to an eleven nineteen leukemia (ENL) ligand PI via a Linker:
PI-Linker-EL;
and enantiomers, diastereoisomers and pharmaceutically acceptable salts thereof, wherein:
PI comprises an ENL ligand selected from the group consisting of: 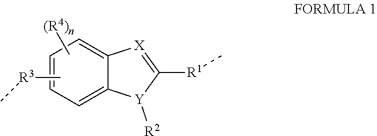
wherein
the “Linker” moiety of the bivalent compound is attached independently to R¹ or R³
X and Y are independently selected from C, O or N;
R¹ is selected from H, halogen, OR⁵, SR⁵, C₁-C₈ alkylene NR⁵R⁶, CH₂CH₂NR⁵R⁶, NR⁵R⁶, C(O)R⁵, C(O)OR⁵, C(S)OR⁵, C(O)NR⁵R⁶, S(O)R⁵, S(O)₂R⁵, S(O)₂NR⁵R⁶, NR⁷C(O)OR⁶, NR⁷C(O)R⁶, NR⁷S(O)R⁶, NR⁷S(O)₂R⁶, or unsubstituted or optionally substituted C₁-C₈ alkyl, C₁-C₈ haloalkyl, C₁-C₈ hydroxyalkyl, C₃-C₁₀ cycloalkyl, C₃-C₁₀ heterocyclyl, optionally substituted C₁-C₈alkoxyC₁-C₈alkyl, optionally substituted C₁-C₈alkylaminoC₁-C₈alkyl;
R² is independently selected from hydrogen, halogen, oxo, CN, NO₂, OR⁸, SR⁸, NR⁸R⁹, C(O)R⁸, C(O)OR⁸, C(S)OR⁸, C(O)NR⁸R⁹, S(O)R⁸, S(O)₂R⁸, S(O)₂NR⁸R⁹, NR¹⁰C(O)OR⁹, NR¹⁰C(O)R⁹, NR¹⁰S(O)R⁹, NR¹⁰S(O)₂R⁹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₃-C₈ heterocyclyl, optionally substituted aryl, and optionally substituted heteroaryl;
R³ is unsubstituted or optionally substituted with one or more groups selected from hydrogen, halogen, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR¹¹R¹², C(O)R¹¹, C(O)OR¹¹, C(O)NR¹¹R¹², S(O)R¹¹, S(O)₂R¹¹, S(O)₂NR¹¹R¹², NR¹³C(O)OR¹², NR¹³C(O)R¹², NR¹³S(O)R¹², NR¹³S(O)₂R¹², optionally substituted C₆-C₁₀ aryl and optionally substituted C₅-C₁₀ heteroaryl;
each R⁴ is independently selected from null, hydrogen, halogen, oxo, CN, NO₂, OR¹⁴, SR¹⁴, NR¹⁴R¹⁵, OCOR¹⁴, OCO₂R¹⁴, OCONR¹⁴R¹⁵, COR¹⁴, CO₂R¹⁵, CONR¹⁴R¹⁵, SOR¹⁴, SO₂R¹⁴, SO₂NR¹⁴R¹⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;
R⁵, R⁶, R⁷, R⁸, R⁹, R¹⁰ R¹¹, R¹², R¹³ R¹⁴, R¹⁵ are independently selected from H, C₁-C₈ alkyl, C₁-C₈ haloalkyl, C₁-C₈ hydroxyalkyl, C₃-C₁₀ cycloalkyl, C₃-C₁₀ heterocyclyl, C(O) C₁-C₈ alkyl, C(O) C₁-C₈ haloalkyl, C(O) C₁-C₈ hydroxyalkyl, C(O) C₃-C₁₀ cycloalkyl, C(O) C₃-C₁₀ heterocyclyl, optionally substituted C₆-C₁₀ aryl or C₅-C₁₀ heteroaryl;
R⁵ and R⁶, R⁶ and R⁷, R⁸ and R⁹, R⁸ and R¹⁰, R⁹ and R¹⁰, R¹¹ and R¹², R¹¹ and R¹³, R¹² and R¹³, R¹⁴ and R¹⁵, together with the nitrogen atom to which they connected can independently form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring; and
n is independently selected from 0, 1, 2, 3, 4 and 5; 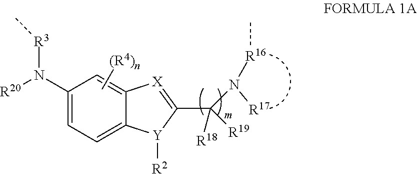
wherein
the “Linker” moiety of the bivalent compound is attached independently to R³ or R¹⁶;
X and Y are independently selected from C, O or N;
the definitions of R², R³, R⁴ are the same as for FORMULA 1;
R¹⁶, R¹⁷ is selected from hydrogen, C₁-C₈ alkyl, C₁-C₈ haloalkyl, C₁-C₈ hydroxyalkyl, C₃-C₁₀ cycloalkyl, C₃-C₁₀ heterocycloalkyl, C₆-C₁₀ aryl, C₅-C₁₀ heteroaryl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, C(O)C₆-C₁₀ aryl, C(O)C₅-C₁₀ heteroaryl;
or
R¹⁶ and R¹⁷ together with the nitrogen atom to which they connected can independently form form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring.
R¹⁸, R¹⁹ are independently selected from hydrogen, halogen, CN, OH, NH₂, optionally substituted C₁-C₈ alkyl, optionally substituted C₃-C₈ cycloalkyl, optionally substituted C₃-C₈ cycloalkoxy, optionally substituted C₁-C₈alkoxyC₁-C₈alkyl, optionally substituted C₁-C₈alkylaminoC₁-C₈alkyl, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
R²⁰ is selected from hydrogen, optionally substituted C₁-C₈ alkyl, optionally substituted C₃-C₈ cycloalkyl, optionally substituted C₃-C₈ cycloalkoxy, optionally substituted C₃-C₈ heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl and
m, n, are independently selected from 0, 1, 2, 3, and 4; 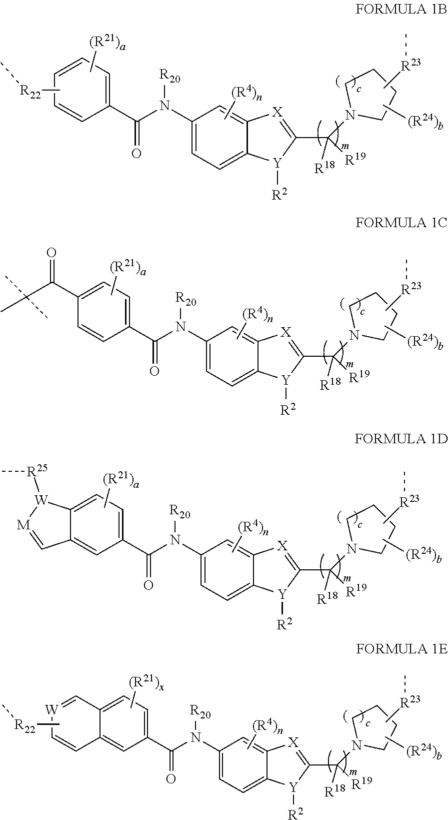
wherein
the “Linker” moiety of the bivalent compound is attached independently to R²², R²³, R²⁵;
X and Y are independently selected from C, O or N;
M and W are independently selected from C or N;
the definitions of R², R⁴, R¹⁸, R¹⁹, R²⁰ are the same as for FORMULA 1A;
each R²¹ is independently selected from null, hydrogen, halogen, oxo, CN, NO₂, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;
R²² is unsubstituted or optionally substituted with one or more groups selected from halo, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR²⁶R²⁷, C₁-C₈NR²⁶R²⁷, C(O)R²⁶, C(O)OR²⁶, C(O)NR²⁶R²⁷, S(O)R²⁶, S(O)₂R²⁶, S(O)₂NR²⁶R²⁷, NR²⁶C(O)OR²⁷, NR²⁸C(O)R²⁷, NR²⁸S(O)R²⁷, NR²⁸S(O)₂R²⁷;
R²³ is unsubstituted or optionally substituted with one or more groups selected from halo, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR²⁹R³⁰, C(O)R²⁹, C(O)OR²⁹, C(O)NR²⁹R³⁰, S(O)R²⁹, S(O)₂R²⁹, S(O)₂NR²⁹R³⁰, NR³¹C(O)OR²⁹, NR³¹C(O)R²⁹, NR³¹S(O)R²⁹, NR³¹S(O)₂R²⁹;
each R²⁴ is independently selected from null, hydrogen, halogen, oxo, CN, NO₂, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;
R²⁵ is unsubstituted or optionally substituted with one or more groups selected from halo, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR³²R³³, C(O)R³², C(O)OR³², C(O)NR³²R³³, S(O)R³², S(O)₂R³², S(O)₂NR³²R³³, NR³⁴C(O)OR³², NR³⁴C(O)R³², NR³⁴S(O)R³², NR³⁴S(O)₂R³²;
R²⁶, R²⁷, R²⁸, R²⁹, R³⁰, R³¹, R³², R³³, R³⁴ are independently selected from H, C₁-C₈ alkyl, C₁-C₈ haloalkyl, C₁-C₈ hydroxyalkyl, C₃-C₁₀ cycloalkyl, C₃-C₁₀ heterocyclyl, C(O) C₁-C₈ alkyl, C(O) C₁-C₈ haloalkyl, C(O) C₁-C₈ hydroxyalkyl, C(O) C₃-C₁₀ cycloalkyl, C(O) C₃-C₁₀ heterocyclyl, optionally substituted C₆-C₁₀ aryl or C₅-C₁₀ heteroaryl;
R²⁶ and R²⁷, R²⁷ and R²⁸, R²⁹ and R³⁰, R²⁹ and R³¹, R³² and R³³, R³² and R³⁴, together with the nitrogen atom to which they connected can independently form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring;
m, n, a, b are independently selected from 0, 1, 2, 3, and 4; and
c is independently selected from 0, 1, 2, 3, 4, 5 and 6; 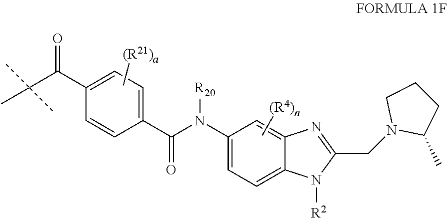
wherein
the “Linker” moiety of the bivalent compound is attached to the carbonyl group indicated with dotted line;
the definitions of R², R⁴, R²⁰, R²¹ are the same as for FORMULA 1B; and
n, a are independently selected from 0, 1, 2, 3, and 4; 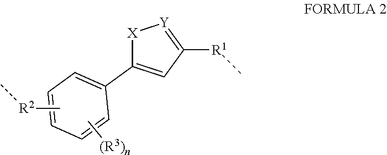
wherein
the “Linker” moiety of the bivalent compound is attached independently to R¹ or R²;
X and Y are independently selected from C, O or N;
R¹ is selected from hydrogen, halogen, OR⁴, SR⁴, C₁-C₈ alkylene NR⁴R⁵, C(O)R⁴, C(O)OR⁴, C(S)OR⁴, C(O)NR⁴R⁵, S(O)R⁴, S(O)₂R⁴, S(O)₂NR⁴R⁵, NR⁶C(O)OR⁴, NR⁶C(O)R⁴, NR⁶S(O)R⁴, NR⁶S(O)₂R⁴, or unsubstituted or optionally substituted C₁-C₈ alkyl, C₁-C₈ haloalkyl, C₁-C₈ hydroxyalkyl, C₃-C₁₀ cycloalkyl, C₃-C₁₀ heterocyclyl, or fused C₃-C₁₀ cycloalkyl, C₃-C₁₀ heterocyclyl;
R² is selected from hydrogen, halogen, CN, NO₂, or unsubstituted or optionally substituted C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR⁷R⁸, C(O)R⁷, C(O)OR⁷, C(O)NR⁷R⁸, S(O)R⁷, S(O)₂R⁷, S(O)₂NR⁷R⁸, NR⁹C(O)OR⁷, NR⁹C(O)R⁷, NR⁹S(O)R⁷, NR⁹S(O)₂R⁷, optionally substituted C₆-C₁₀ aryl and optionally substituted C₅-C₁₀ heteroaryl;
each R³ is independently selected from null, hydrogen, halogen, oxo, OH, CN, NO₂, OR¹⁰, SR¹⁰, NR¹⁰R¹¹, OCOR¹⁰, OCO₂R¹⁰, OCONR¹⁰R¹¹, COR¹⁰, CO₂R¹⁰, CONR¹⁰R¹¹, SOR¹⁰, SO₂R¹⁰, SO₂NR¹⁰R¹¹, NR¹²C(O)OR¹⁰, NR¹²C(O)R¹⁰, NR¹²S(O)R¹⁰, NR¹²S(O)₂R¹⁰, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;
wherein
R⁴, R⁵, R⁶, R⁷, R⁸, R⁹, R¹⁰ R¹¹, R¹² are independently selected from H, C₁-C₈ alkyl, C₁-C₈ haloalkyl, C₁-C₈ hydroxyalkyl, C₃-C₁₀ cycloalkyl, C₃-C₁₀ heterocyclyl, C(O) C₁-C₈ alkyl, C(O) C₁-C₈ haloalkyl, C(O) C₁-C₈ hydroxyalkyl, C(O) C₃-C₁₀ cycloalkyl, C(O) C₃-C₁₀ heterocyclyl, optionally substituted C₆-C₁₀ aryl or C₅-C₁₀ heteroaryl;
R⁴ and R⁵, R⁴ and R⁶, R⁷ and R⁸, R⁷ and R⁹, R¹⁰ and R¹¹, R¹⁰ and R¹², together with the nitrogen atom to which they connected can independently form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring; and
n is independently selected from 0, 1, 2, 3, 4; 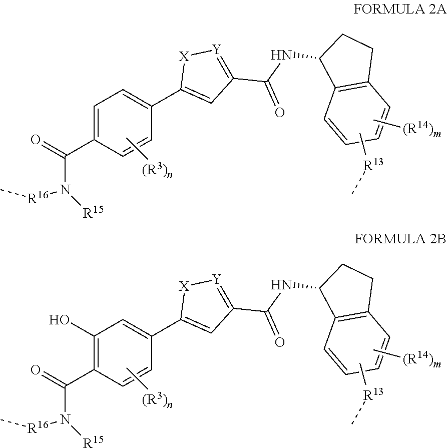
wherein
the “Linker” moiety of the bivalent compound is attached independently to R¹³ or R¹⁶;
X and Y are independently selected from C, O or N;
the definition of R³ is the same as for FORMULA 2;
R¹³ is selected from hydrogen, halogen OR¹⁷, SR¹⁷, C₁-C₈ alkylene NR¹⁷R¹⁸, C(O)R¹⁷, C(O)OR¹⁷, C(S)OR¹⁷, C(O)NR¹⁷R¹⁸, S(O)R¹⁷, S(O)₂R¹⁷, S(O)₂NR¹⁷R¹⁸, NR¹⁹C(O)OR¹⁷, NR¹⁹C(O)R¹⁷, NR¹⁹S(O)R¹⁷, NR¹⁹S(O)₂R¹⁷, or unsubstituted or optionally substituted C₁-C₈ alkyl, C₁-C₈ haloalkyl, C₁-C₈ hydroxyalkyl, C₃-C₁₀ cycloalkyl, C₃-C₁₀ heterocyclyl;
each R¹⁴ is independently selected from unsubstituted or optionally substituted with one or more groups selected from hydrogen, halogen, CN, NO₂, C₁-C₈ alkyl, C₁-C₈ haloalkyl, C₁-C₈ hydroxyalkyl, C₃-C₁₀ cycloalkyl, C₃-C₁₀ heterocyclyl, C(O)C₁-C₈ alkyl, C(O)C₁-C₈ haloalkyl, C(O)C₁-C₈ hydroxyalkyl, C(O)C₃-C₁₀ cycloalkyl, C(O)C₃-C₁₀ heterocyclyl, NR²⁰R²¹, C(O)R²⁰, C(O)OR²⁰, C(O)NR²⁰R²¹, S(O)R²⁰, S(O)₂R²⁰, S(O)₂NR²⁰R²¹, NR²²C(O)OR²⁰, NR²²C(O)R²⁰, NR²²S(O)R²⁰, NR²²S(O)₂R²⁰, optionally substituted C₆-C₁₀ aryl and optionally substituted C₅-C₁₀ heteroaryl;
R¹⁵ is selected from hydrogen, optionally substituted C₁-C₈ alkyl, optionally substituted C₃-C₈ cycloalkyl, optionally substituted C₃-C₈ cycloalkoxy, optionally substituted C₃-C₈ heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
R¹⁶ is selected from null, hydrogen, halogen, oxo, CN, NO₂, OR²³, SR²³, NR²³R²⁴, OCOR²³, OCO₂R²³, OCONR²³R²⁴, COR²³, CO₂R²³, CONR²³R²⁴, SOR²³, SO₂R²³, SO₂NR²³R²⁴, NR²⁵C(O)OR²³, NR²⁵C(O)R²³, NR²⁵S(O)R²³, NR²⁵S(O)₂R²³, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted C₃-C₈ cycloalkoxy, optionally substituted C₄-C₈ heterocyclyl, optionally substituted aryl, and optionally substituted heteroaryl;
wherein
R¹⁷, R¹⁸, R¹⁹, R²⁰, R²¹, R²², R²³, R²⁴, R²⁵ are independently selected from H, C₁-C₈ alkyl, C₁-C₈ haloalkyl, C₁-C₈ hydroxyalkyl, C₃-C₁₀ cycloalkyl, C₃-C₁₀ heterocyclyl, C(O) C₁-C₈ alkyl, C(O) C₁-C₈ haloalkyl, C(O) C₁-C₈ hydroxyalkyl, C(O) C₃-C₁₀ cycloalkyl, C(O) C₃-C₁₀ heterocyclyl, optionally substituted C₆-C₁₀ aryl or C₅-C₁₀ heteroaryl;
R¹⁷ and R¹⁸, R¹⁷ and R¹⁹, R²⁰ and R²¹, R²⁰ and R²², R²³ and R²⁴, R²³ and R²⁵, together with the nitrogen atom to which they connected can independently form optionally substituted C₃-C₁₃ heterocyclyl rings, optionally substituted C₃-C₁₃ fused cycloalkyl ring, optionally substituted C₃-C₁₃ fused heterocyclyl ring, optionally substituted C₃-C₁₃ bridged cycloalkyl ring, optionally substituted C₃-C₁₃ bridged heterocyclyl ring, optionally substituted C₃-C₁₃ spiro cycloalkyl ring, and optionally substituted C₃-C₁₃ spiro heterocyclyl ring; and
m, n is independently selected from 0, 1, 2, 3, 4; 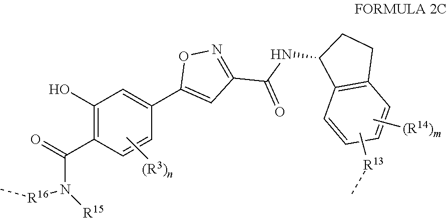
wherein
the “Linker” moiety of the bivalent compound is attached independently to R¹³ or R¹⁶; and
the definitions of R³, R¹³, R¹⁴, R¹⁵ an R¹⁶ are the same as for FORMULA 2A and 2C; 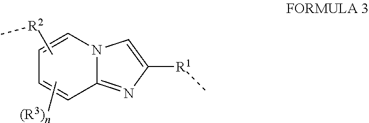
Wherein
the “Linker” moiety of the bivalent compound is attached independently to R¹ or R²
the definitions of R¹, R² and R³ are the same as for FORMULA 2; and 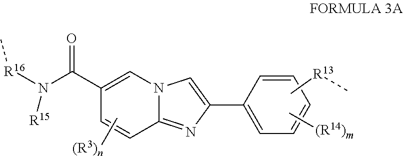
wherein
the “Linker” moiety of the bivalent compound is attached independently to R¹³ or R¹⁶
the definitions of R³, R¹³, R¹⁴, R¹⁵ and R¹⁶ are the same as for FORMULA 2A;
n is selected from 0, 1, 2, 3; and
m is selected from 0, 1, 2, 3, 4.

2. The bivalent compound of claim 1 wherein the ENL ligand is selected from the group consisting of:

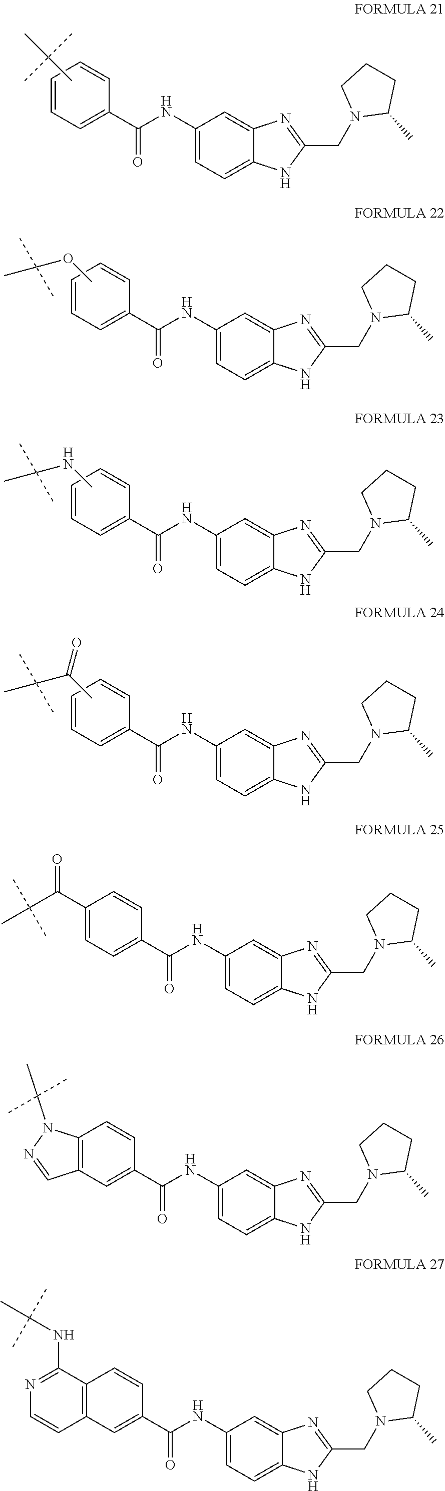

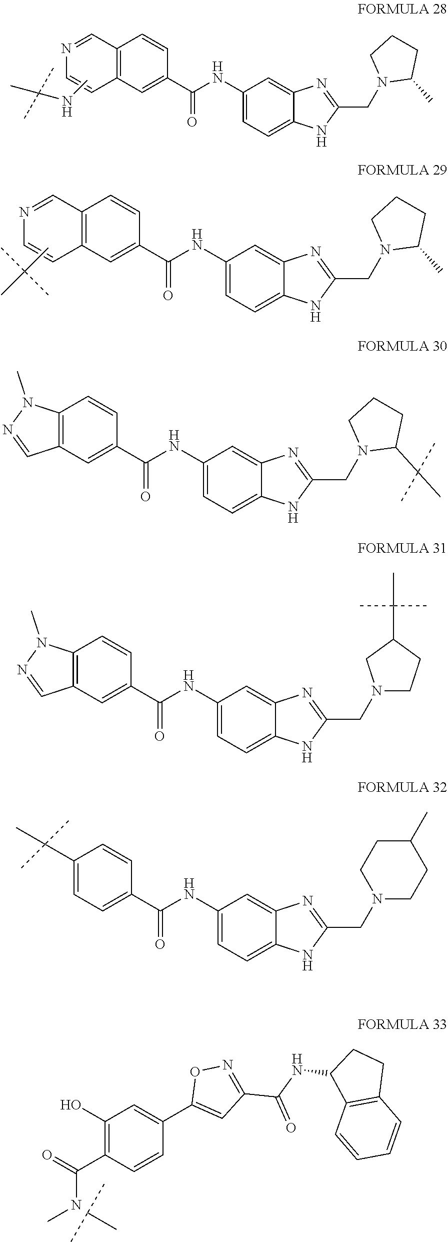

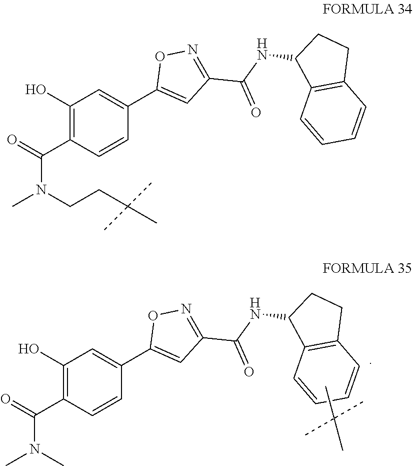

3. The bivalent compound of claim 1, wherein the degrader/disruption tag EL is selected from the group consisting of: 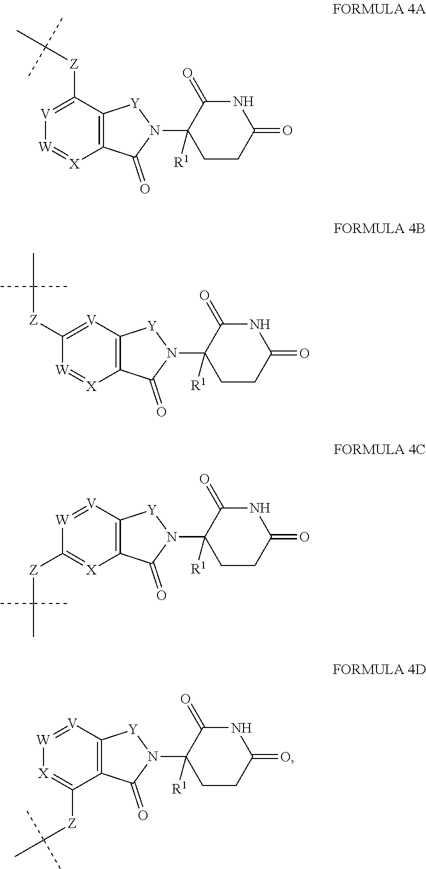
wherein
V, W, and X are independently selected from CR² and N;
Y is selected from CO, CR³R⁴, and N═N;
Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferly, Z is selected from null, CH₂, CH═CH, C≡C, NH and O;
R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;
R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and
R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl; 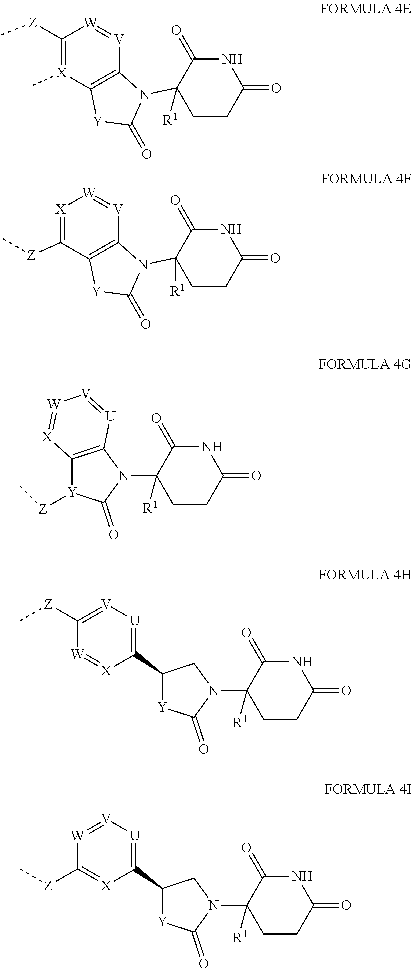
wherein
U, V, W, and X are independently selected from CR² and N;
Y is selected from CR³R⁴, NR³ and O; preferably, Y is selected from CH₂, NH, NCH₃ and O;
Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;
R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;
R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and
R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl; 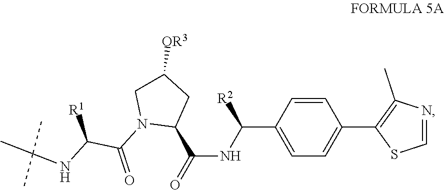
wherein
R¹ and R² are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₇ cycloalkyl, optionally substituted 3-7 membered heterocyclyl, optionally substituted C₂-C₈ alkenyl, and optionally substituted C₂-C₈ alkynyl; and
R³ is hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)C₃-C₇ cycloalkyl, optionally substituted C(O)(3-7 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)OC₃-C₇ cycloalkyl, optionally substituted C(O)O(3-7 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)NC₃-C₇ cycloalkyl, optionally substituted C(O)N(3-7 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂; 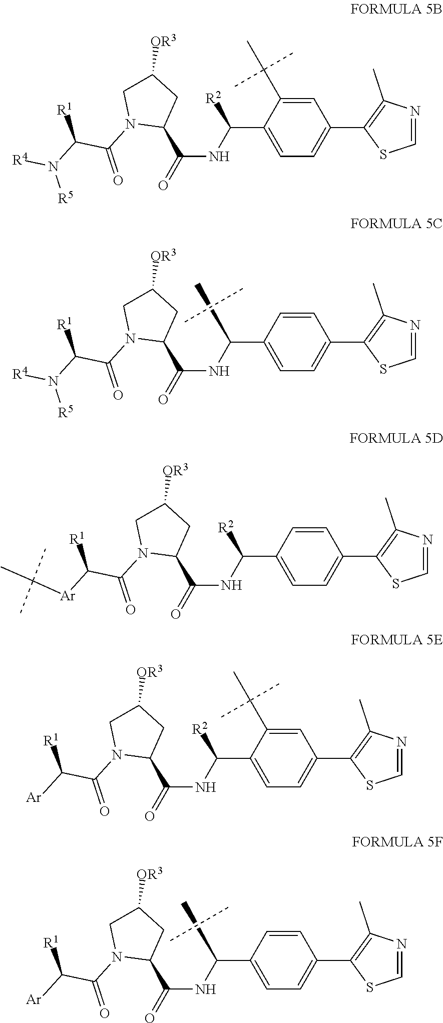
wherein
R¹ and R² are independently selected from hydrogen, halogen, OH, NH₂, CN, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₇ cycloalkyl, optionally substituted 3-7 membered heterocyclyl, optionally substituted C₂-C₈ alkenyl, and optionally substituted C₂-C₈ alkynyl; (preferably, R¹ is selected from iso-propyl or tert-butyl; and R² is selected from hydrogen or methyl);
R³ is hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)C₃-C₇ cycloalkyl, optionally substituted C(O)(3-7 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)OC₃-C₇ cycloalkyl, optionally substituted C(O)O(3-7 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)NC₃-C₇ cycloalkyl, optionally substituted C(O)N(3-7 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂; and
R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; wherein
R⁶ and R⁷ are independently selected from hydrogen, optionally substituted C₁-C₈ alkyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; or
R⁴ and R⁵; R⁶ and R⁷ together with the atom to which they are connected form a 4-8 membered cycloalkyl or heterocyclyl ring;
Ar is selected from aryl and heteroaryl, each of which is optionally substituted with one or more substituents independently selected from F, Cl, CN, NO₂, OR⁸, NR⁸R⁹, COR⁸, CO₂R⁸, CONR⁸R⁹, SOR⁸, SO₂R⁸, SO₂NR⁹R¹⁰, NR⁹COR¹⁰, NR⁸C(O)NR⁹R¹⁰, NR⁹SOR¹⁰, NR⁹SO₂R¹⁰, optionally substituted C₁-C₆ alkyl, optionally substituted C₁-C₆ alkoxyalkyl, optionally substituted C₁-C₆ haloalkyl, optionally substituted C₁-C₆ hydroxyalkyl, optionally substituted C₁-C₆alkylaminoC₁-C₆alkyl, optionally substituted C₃-C₇ cycloalkyl, optionally substituted 3-7 membered heterocyclyl, optionally substituted C₂-C₆ alkenyl, optionally substituted C₂-C₆ alkynyl, optionally substituted aryl, and optionally substituted C₄-C₅ heteroaryl; wherein
R⁸, R⁹, and R¹⁰ are independently selected from null, hydrogen, optionally substituted C₁-C₆ alkyl, optionally substituted C₂-C₆ alkenyl, optionally substituted C₂-C₆ alkynyl, optionally substituted C₃-C₇ cycloalkyl, optionally substituted 3-7 membered heterocyclyl, optionally substituted aryl, and optionally substituted heteroaryl; or
R⁸ and R⁹; R⁹ and R¹⁰ together with the atom to which they are connected form a 4-8 membered cycloalkyl or heterocyclyl ring; 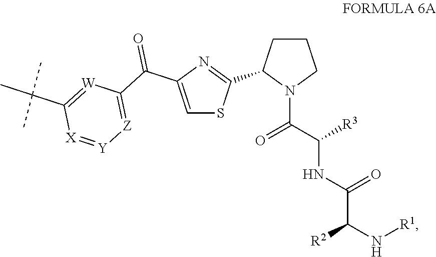
wherein
V, W, X, and Z are independently selected from CR⁴ and N;
R¹, R², R³, and R⁴ are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl; and 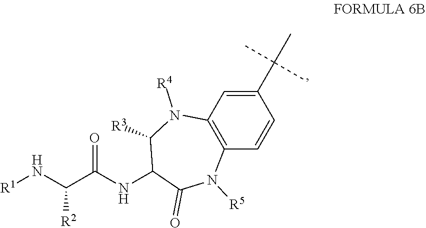
wherein
R¹, R², and R³ are independently selected from hydrogen, halogene, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl;
R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted aryl-C₁-C₈alkyl, optionally substituted 3-8 membered cycloalkyl, optionally substituted 3-8 membered heterocyclyl, optionally substituted aryl, and optionally substituted heteroaryl; wherein
R⁶ and R⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl; or
R⁶ and R⁷ together with the atom to which they are connected form a 4-8 membered cycloalkyl or heterocyclyl ring.

4. The bivalent compound of claim 1, wherein the disruption/degrader tag EL is selected from the group consisting of:

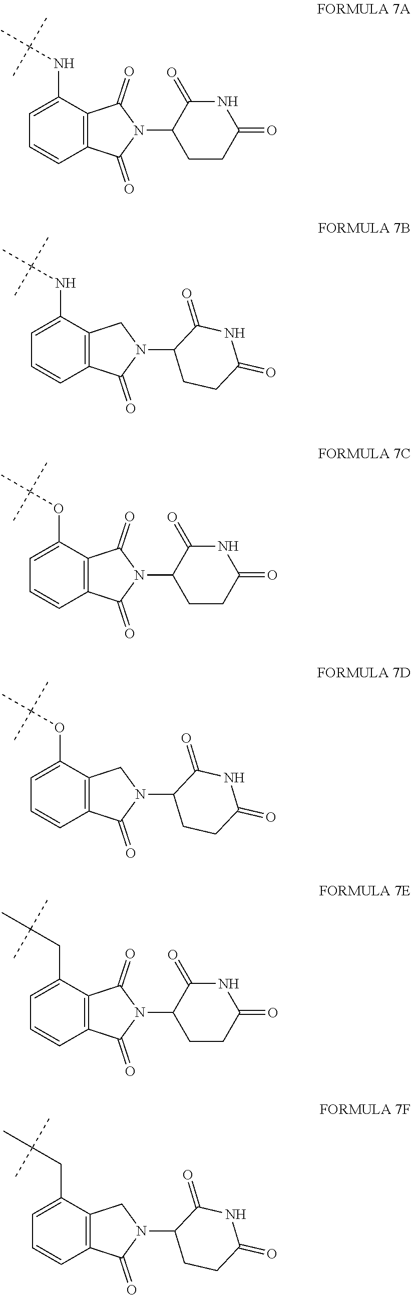

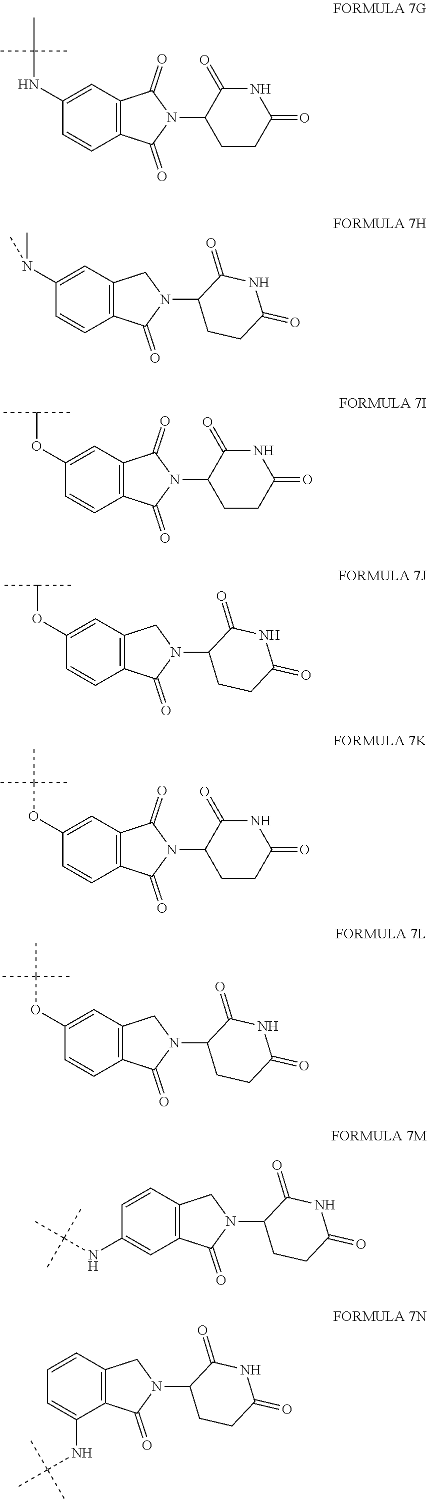

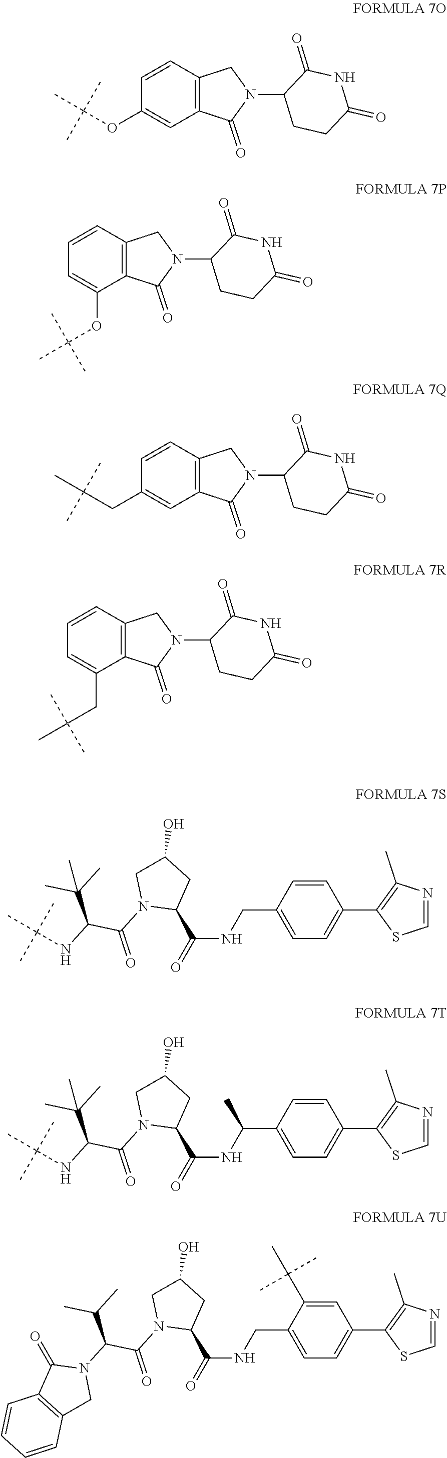

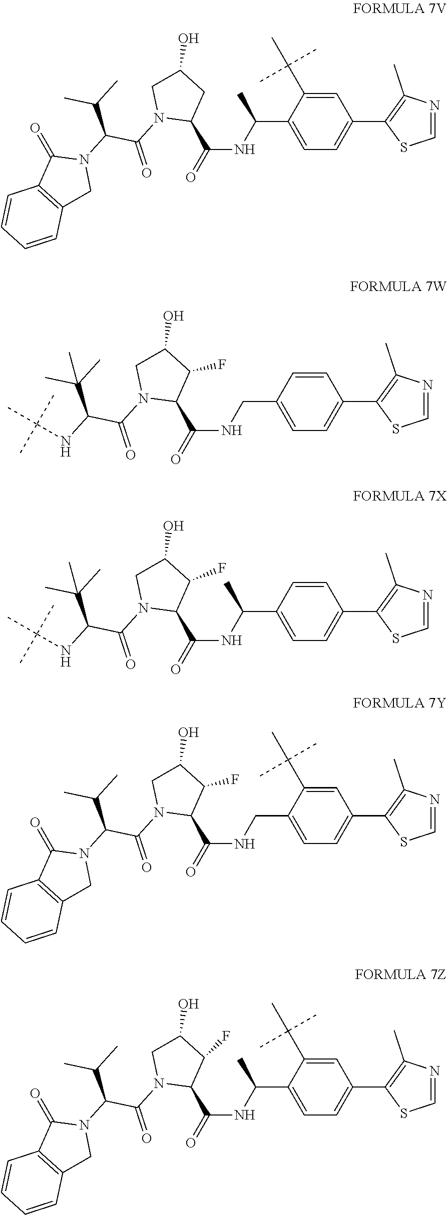

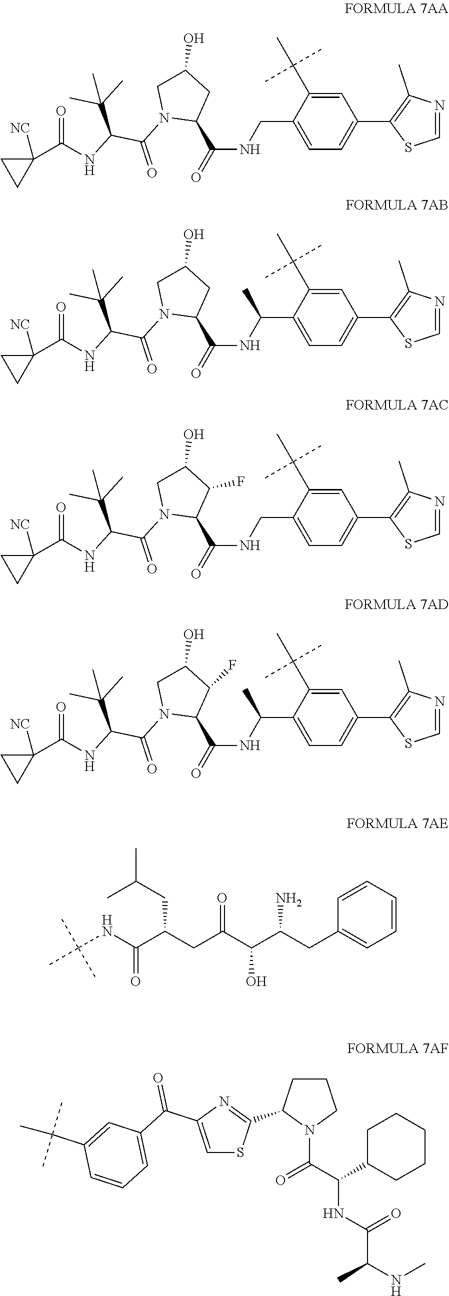

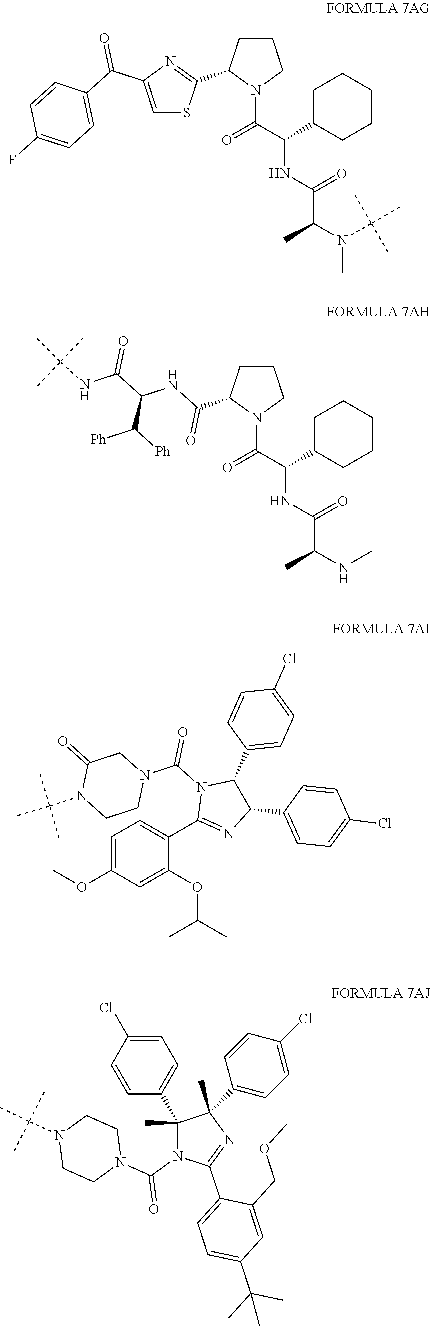

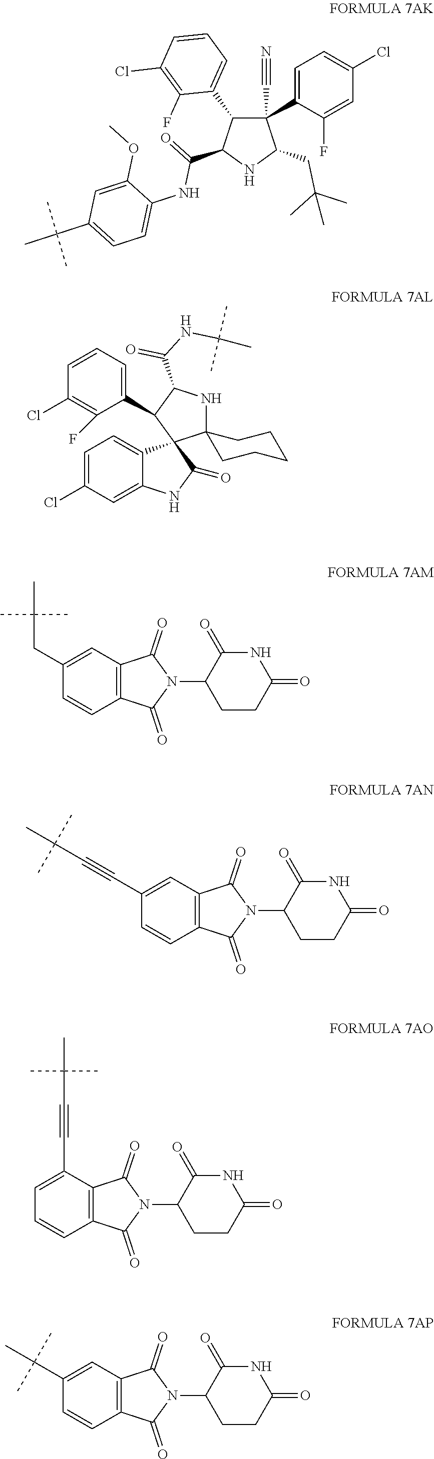

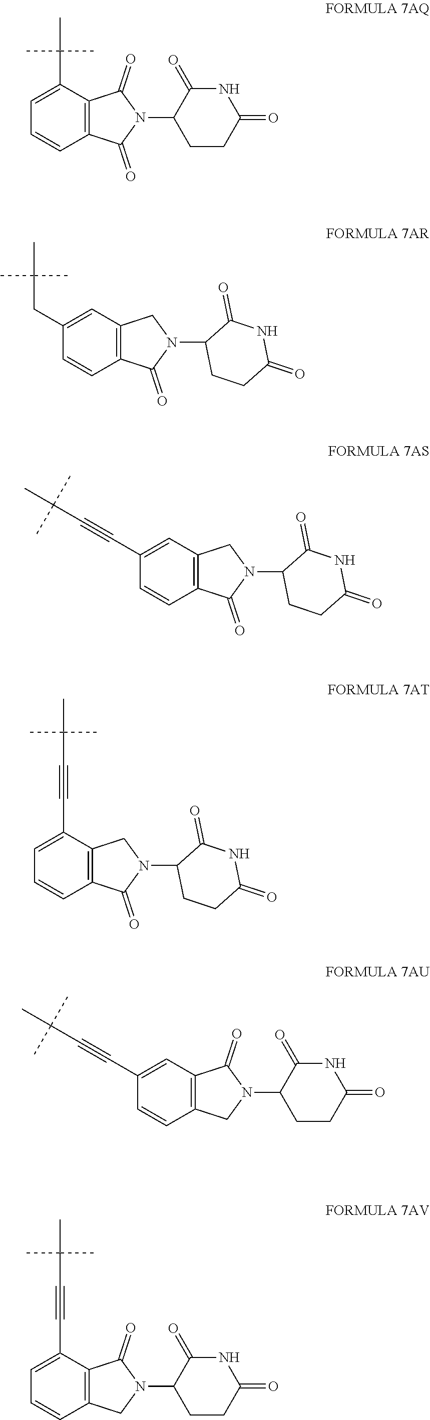

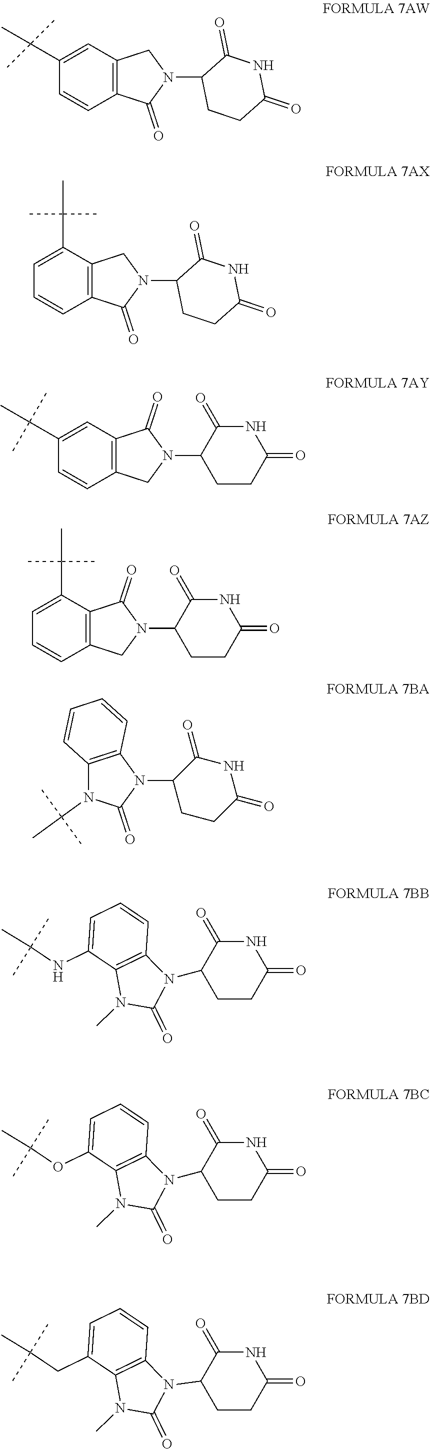

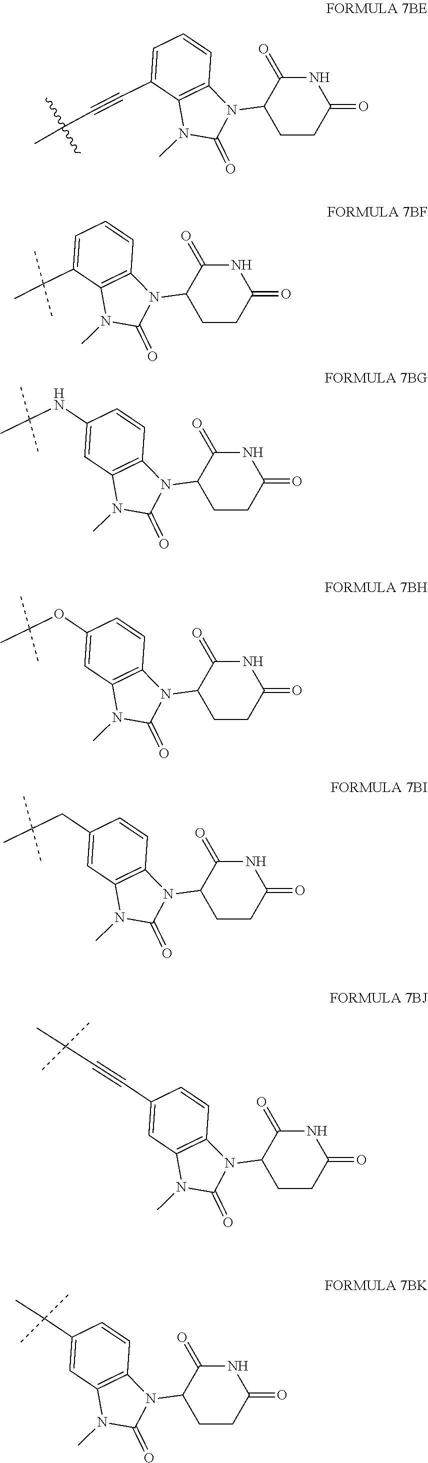

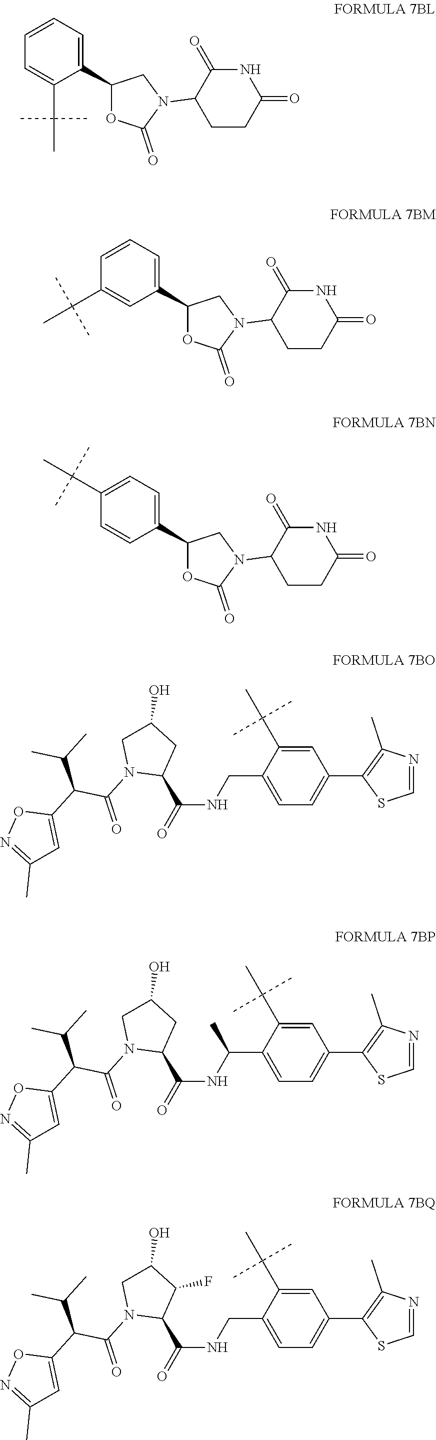

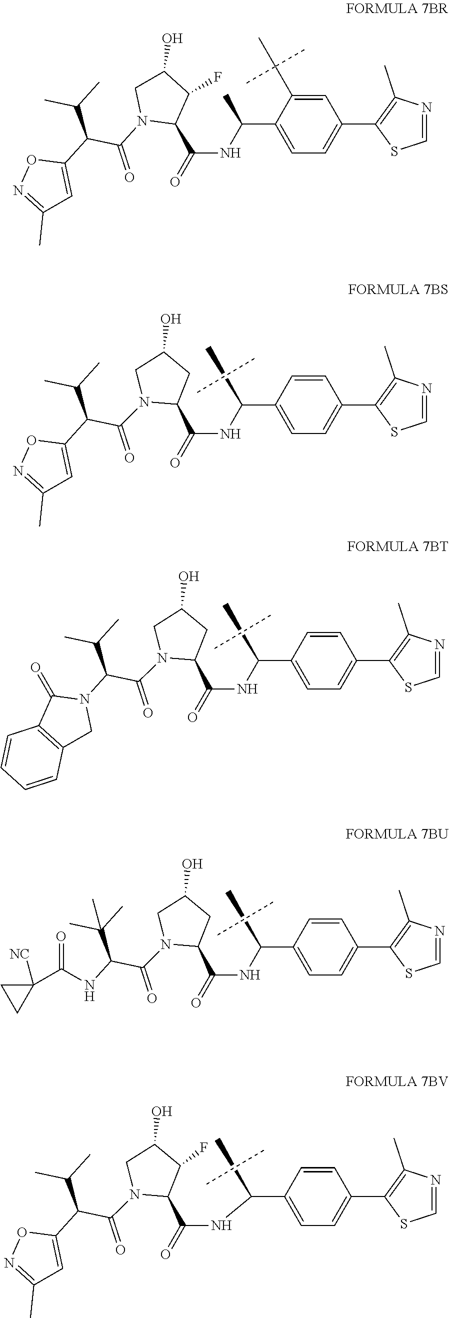

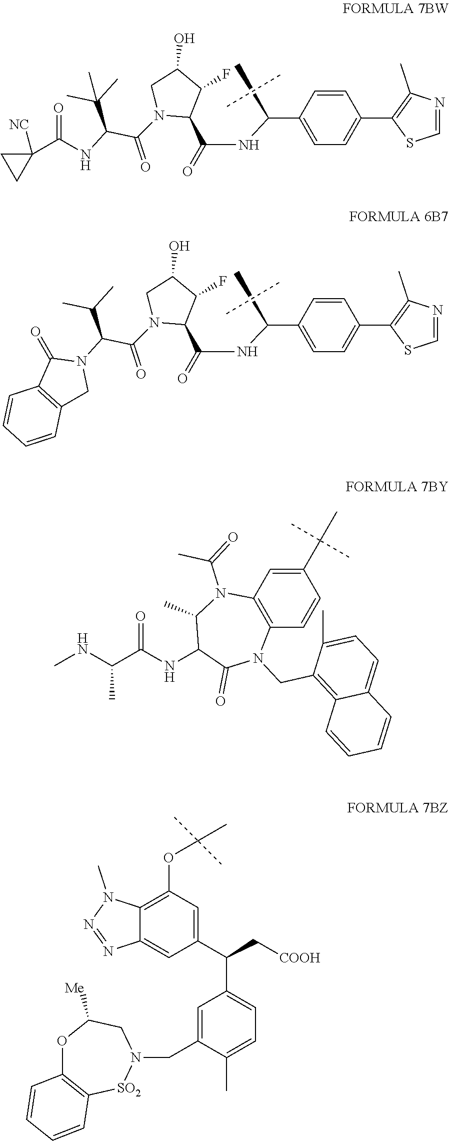

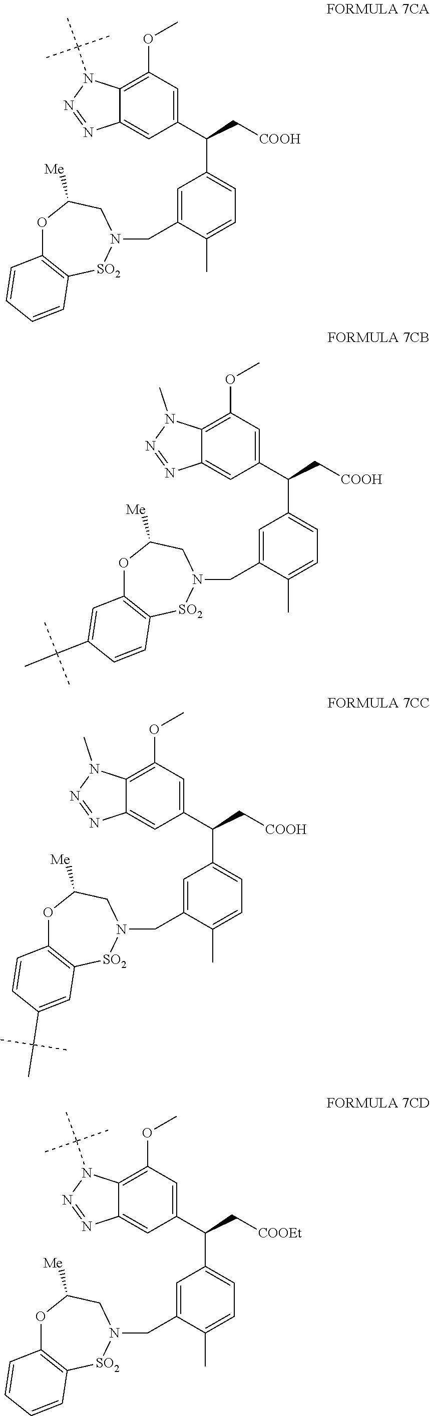

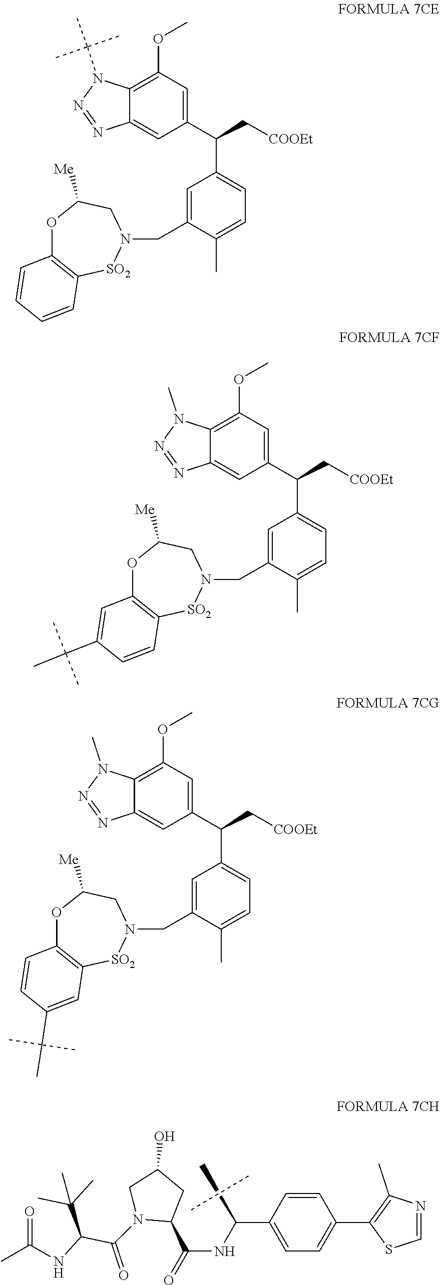

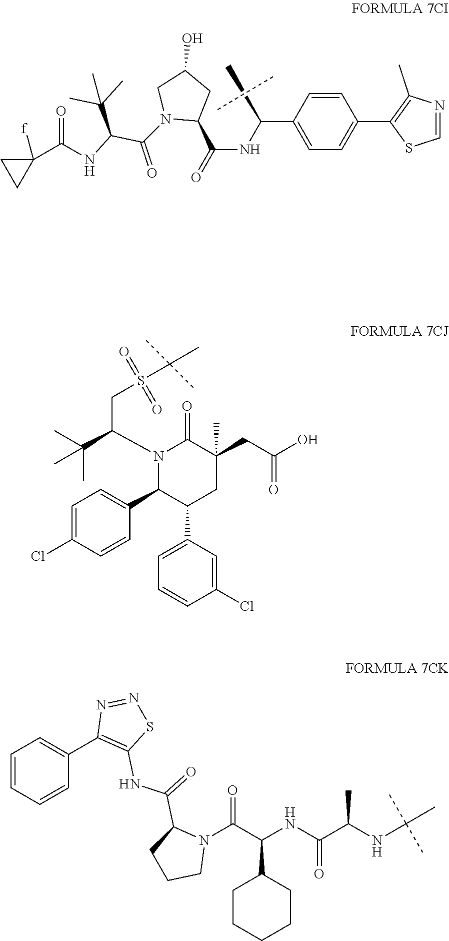

5. The bivalent compound of claim 1, wherein the linker is selected from the group consisting of: 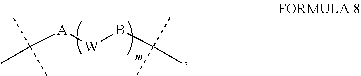
wherein
A, W, and B, at each occurrence, are independently selected from null, CO, CO₂, C(O)NR¹, C(S)NR¹, O, S, SO, SO₂, SO₂NR¹, NR¹, NR¹CO, NR¹CONR², NR¹C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R¹ and R² are independently selected from hydrogen, optionally substituted C₁-C₈ alkyl, optionally substituted C₃-C₈ cycloalkyl, optionally substituted 3-8 membered cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈alkylaminoC₁-C₈alkyl; and
m is 0 to 15; 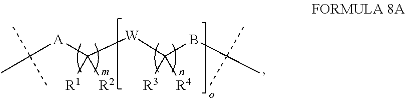
wherein
R¹, R², R³, and R⁴, at each occurrence, are independently selected from hydrogen, halogen, CN, OH, NH₂, optionally substituted C₁-C₈ alkyl, optionally substituted C₃-C₈ cycloalkyl, optionally substituted C₃-C₈ cycloalkoxy, optionally substituted 3-8 membered membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
A, W, and B, at each occurrence, are independently selected from null, CO, CO₂, C(O)NR⁵, C(S)NR⁵, O, S, SO, SO₂, SO₂NR⁵, NR⁵, NR⁵CO, NR⁵CONR⁶, NR⁵C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R⁵ and R⁶ are independently selected from hydrogen, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈alkylaminoC₁-C₈alkyl;
m is 0 to 15;
n, at each occurrence, is 0 to 15; and
o is 0 to 15; 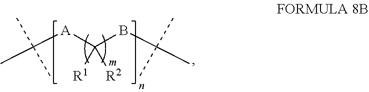
wherein
R¹ and R², at each occurrence, are independently selected from hydrogen, halogen, CN, OH, NH₂, and optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, or C₁-C₈alkylaminoC₁-C₈alkyl;
A and B, at each occurrence, are independently selected from null, CO, CO₂, C(O)NR³, C(S)NR³, O, S, SO, SO₂, SO₂NR³, NR³, NR³CO, NR³CONR⁴, NR³C(S), and optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, or C₃-C₁₃ spiro heterocyclyl; wherein
R³ and R⁴ are independently selected from hydrogen, and optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, or C₁-C₈alkylaminoC₁-C₈alkyl;
each m is 0 to 15; and
n is 0 to 15; 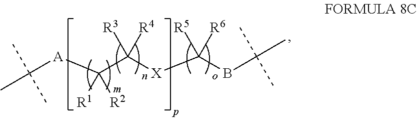
wherein
X is selected from O, NH, and NR⁷;
R¹, R², R³, R⁴, R⁵, and R⁶, at each occurrence, are independently selected from hydrogen, halogen, CN, OH, NH₂, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH—CH₂—NH—CH₂, CH₂—NH—CH₂, CO₂, C(O)NR⁷, C(S)NR⁷, O, S, SO, SO₂, SO₂NR⁷, NR⁷, NR⁷CO, NR⁷CONR⁸, NR⁷C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R⁷ and R⁸ are independently selected from hydrogen, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
m, at each occurrence, is 0 to 15;
n, at each occurrence, is 0 to 15;
o is 0 to 15; and
p is 0 to 15.

6. The bivalent compound of claim 1, wherein the linker is selected from the group consisting of a ring selected from the group consisting of a 3 to 13 membered ring; a 3 to 13 membered fused ring; a 3 to 13 membered bridged ring; and a 3 to 13 membered spiro ring; and pharmaceutically acceptable salts thereof.

7. The bivalent compound of claim 1, wherein the linker is selected from the group consisting of:

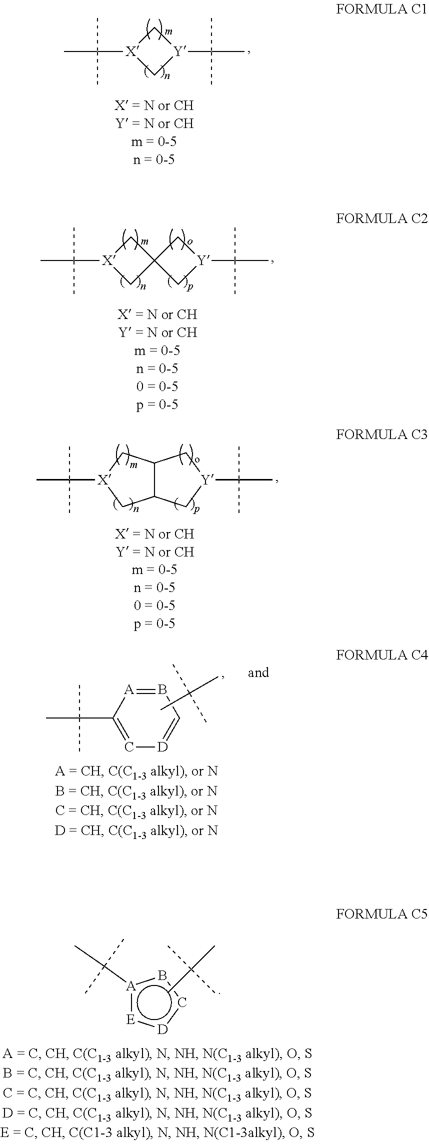

8. A bivalent compound selected from the group consisting of:
LQ076-46, LQ076-47, LQ076-48, LQ076-49, LQ076-50, LQ076-51, LQ076-52, LQ076-53, LQ076-54, LQ076-55, LQ076-56, LQ076-57, LQ076-58, LQ076-59, LQ076-60, LQ076-61, LQ076-62, LQ076-63, LQ076-64, LQ076-65, LQ076-66, LQ076-67, LQ076-68, LQ076-69, LQ076-70, LQ076-71, LQ076-72, LQ076-73, LQ076-74, LQ076-75, LQ076-76, LQ076-77, LQ076-78, LQ076-79, LQ076-80, LQ076-81, LQ076-82, LQ076-83, LQ076-84, LQ076-85, LQ076-86, LQ076-87, LQ076-88, LQ076-89, LQ076-90, LQ076-91, LQ076-92, LQ076-93, LQ076-94, LQ076-95, LQ076-96, LQ076-97, LQ076-98, LQ076-99, LQ076-100, LQ076-101, LQ076-102, LQ076-103, LQ076-104, LQ076-105, LQ076-106, LQ076-107, LQ076-108, LQ076-109, LQ076-110, LQ076-111, LQ076-112, LQ076-113, LQ076-114, LQ076-115, LQ076-116, LQ076-117, LQ076-118, LQ076-119, LQ076-120, LQ076-121, LQ076-122, LQ076-123, LQ076-124, LQ076-125, LQ076-126, LQ076-127, LQ076-128, LQ076-129, LQ076-130, LQ076-131, LQ076-132, LQ076-133, LQ076-134, LQ076-135, LQ076-136, LQ076-137, LQ076-138, LQ076-139, LQ076-140, LQ076-141, LQ076-142, LQ076-143, LQ076-144, LQ076-145, LQ076-146, LQ076-147, LQ076-148, LQ076-149, LQ076-150, LQ076-151, LQ076-152, LQ076-153, LQ076-154, LQ076-155, LQ076-156, LQ076-157, LQ076-158, LQ076-159, LQ076-160, LQ076-161, LQ076-162, LQ076-163, LQ081-100, LQ081-101, LQ081-102, LQ081-103, LQ081-104, LQ081-105, LQ081-108, LQ081-109, LQ081-122, LQ081-132, LQ081-133, LQ081-146, LQ081-147, LQ081-150, LQ086-31, LQ086-32, LQ086-33, LQ086-34, LQ086-35, LQ086-36, LQ086-38, LQ086-40, LQ086-41, LQ086-76, LQ086-76Na, LQ108-6, LQ108-7, LQ108-8, LQ108-9, LQ108-10, LQ108-11, LQ108-12, LQ108-146, LQ108-147, LQ108-148, LQ108-149, LQ108-150, LQ108-151, LQ108-152, LQ108-153, LQ108-154, LQ108-155, LQ108-156, LQ108-157, LQ118-23, LQ118-24, LQ118-25; LQ108-58, LQ108-60, LQ108-61, LQ108-62, LQ108-63, LQ108-64, LQ108-65, LQ108-66, LQ108-67, LQ108-68, LQ108-69, LQ108-70, LQ108-71, LQ108-72, LQ108-73, LQ108-74, LQ108-75, LQ126-46, LQ126-49, LQ126-50, LQ126-51, LQ126-52, LQ126-53, LQ126-54, LQ126-55, LQ126-56, LQ126-57, LQ126-58, LQ126-59, LQ126-60, LQ126-61, LQ126-62, LQ126-63, LQ126-77, LQ126-78, LQ126-79, LQ126-80, LQ126-81, LQ126-82, LQ126-83, LQ126-84, LQ126-85, LQ126-86, LQ126-87, LQ126-89, LQ126-90, LQ126-91, LQ126-92, LQ126-93, LQ126-94, LQ126-95, LQ126-96, LQ126-97, LQ126-98, LQ126-99, LQ126-100, LQ126-101, LQ126-102, LQ126-103, LQ126-104, LQ126-105, LQ126-106, LQ126-107, LQ126-108, LQ126-109, LQ126-110, LQ126-112, LQ126-113, LQ126-114, LQ126-115, LQ126-116, LQ126-117, LQ126-118, LQ126-120, LQ126-121, LQ126-122, LQ126-123, LQ126-124, LQ126-125, LQ126-126, LQ126-127, LQ126-128, LQ126-130, LQ126-168, LQ126-170, LQ126-171, LQ126-172, LQ126-173, LQ126-174, LQ126-175, LQ126-176, LQ126-177, LQ126-178, LQ126-180, LQ126-181, LQ126-182, LQ126-183, LQ126-184, LQ126-185, LQ126-186, LQ141-1, LQ141-2, LQ141-3, LQ141-4, LQ141-5, LQ141-6, LQ141-7, LQ141-8, LQ141-9, LQ141-10, LQ141-11, LQ141-12, LQ141-13, LQ141-14, LQ141-15, LQ141-16, LQ141-17, LQ141-18, LQ141-19, LQ141-20, LQ141-21, LQ141-22, LQ141-24, LQ141-26, LQ141-27, LQ141-28, LQ141-29, LQ141-33, LQ141-36, LQ141-37, LQ141-38, LQ141-39, LQ141-42, LQ141-43, LQ141-44, LQ141-45, LQ141-46, LQ141-47, LQ141-48, LQ141-49, LQ141-52, LQ141-57, and enantiomers, diastereoisomers and pharmaceutically acceptable salts thereof.

9. A bivalent compound selected from the group consisting of:
N¹-(11-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-11-oxoundecyl)-N⁴-(2-(((S)-2-methylpyrrolidin-1-yl)methyl)-1H-benzo[d]imidazol-5-yl)terephthalamide (LQ076-122);
N¹-(11-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-11-oxoundecyl)-N⁴-(2-(((S)-2-methylpyrrolidin-1-yl)methyl)-1H-benzo[d]imidazol-5-yl)terephthalamide (LQ081-108); and
N¹-(12-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-12-oxododecyl)-N⁴-(2-(((S)-2-methylpyrrolidin-1-yl)methyl)-1H-benzo[d]imidazol-5-yl)terephthalamide (LQ081-109), and enantiomers and pharmaceutically acceptable salts thereof.

10. A method of treating an ENL-mediated disease, comprising administering to a subject in need thereof, a bivalent compound according to claim 1.

11. The method of claim 9, wherein the ENL-mediated disease is selected from the group consisting of solid and liquid cancers, chronic infections that produce exhausted immune response infection-mediated immune suppression; age-related decline in immune response; and age-related decline in cognitive function and infertility.

12. The method of claim 9, wherein the compound is administered orally, parenterally, intradermally, subcutaneously, topically, and/or rectally.

13. The method of claim 9, wherein the subject is treated for cancer and is administered one or more of surgery, chemotherapy, radiation therapy, hormone therapy or immunotherapy.

14. A method of treating a mixed lineage leukemia, comprising administering to a subject in need thereof, a bivalent compound according to claim 1.

15. A bivalent compound comprising a degrader/disruption tag EL conjugated to an eleven nineteen leukemia (ENL) ligand PI via a Linker:
PI-Linker-EL,
and enantiomers, diastereoisomers and pharmaceutically acceptable salts thereof, wherein:
PI comprises the following ENL ligand: 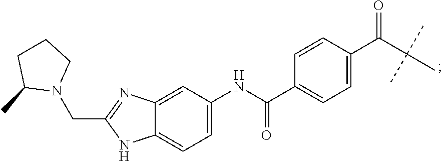
degrader/disruption tag EL is selected from the group consisting of: 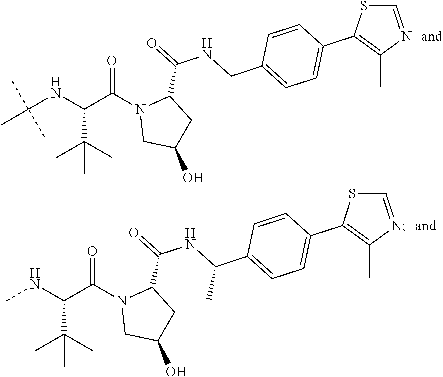
the linker is selected from the group consisting of: 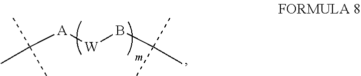
wherein
A, W, and B, at each occurrence, are independently selected from null, CO, CO₂, C(O)NR¹, C(S)NR¹, O, S, SO, SO₂, SO₂NR¹, NR¹, NR¹CO, NR¹CONR², NR¹C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R¹ and R² are independently selected from hydrogen, optionally substituted C₁-C₈ alkyl, optionally substituted C₃-C₈ cycloalkyl, optionally substituted 3-8 membered cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈alkylaminoC₁-C₈alkyl; and
m is 0 to 15; 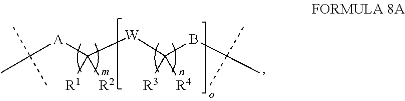
wherein
R¹, R², R³, and R⁴, at each occurrence, are independently selected from hydrogen, halogen, CN, OH, NH₂, optionally substituted C₁-C₈ alkyl, optionally substituted C₃-C₈ cycloalkyl, optionally substituted C₃-C₈ cycloalkoxy, optionally substituted 3-8 membered membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
A, W, and B, at each occurrence, are independently selected from null, CO, CO₂, C(O)NR⁵, C(S)NR⁵, O, S, SO, SO₂, SO₂NR⁵, NR⁵, NR⁵CO, NR⁵CONR⁶, NR⁵C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R⁵ and R⁶ are independently selected from hydrogen, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈alkylaminoC₁-C₈alkyl;
m is 0 to 15;
n, at each occurrence, is 0 to 15; and
o is 0 to 15; 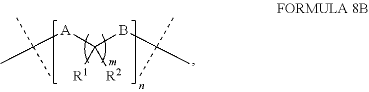
wherein
R¹ and R², at each occurrence, are independently selected from hydrogen, halogen, CN, OH, NH₂, and optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, or C₁-C₈alkylaminoC₁-C₈alkyl;
A and B, at each occurrence, are independently selected from null, CO, CO₂, C(O)NR³, C(S)NR³, O, S, SO, SO₂, SO₂NR³, NR³, NR³CO, NR³CONR⁴, NR³C(S), and optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, or C₃-C₁₃ spiro heterocyclyl; wherein
R³ and R⁴ are independently selected from hydrogen, and optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, or C₁-C₈alkylaminoC₁-C₈alkyl;
each m is 0 to 15; and
n is 0 to 15; 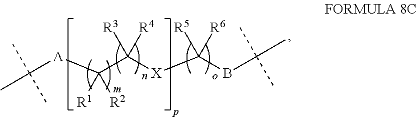
wherein
X is selected from O, NH, and NR⁷;
R¹, R², R³, R⁴, R⁵, and R⁶, at each occurrence, are independently selected from hydrogen, halogen, CN, OH, NH₂, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH—CH₂—NH—CH₂, CH₂—NH—CH₂, CO₂, C(O)NR⁷, C(S)NR⁷, O, S, SO, SO₂, SO₂NR⁷, NR⁷, NR⁷CO, NR⁷CONR⁸, NR⁷C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted 3-8 membered cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl; wherein
R⁷ and R⁸ are independently selected from hydrogen, optionally substituted C₁-C₈ alkyl, optionally substituted 3-8 membered cycloalkyl, optionally substituted C₃-C₈ cycloalkoxy, optionally substituted 3-8 membered heterocyclyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl;
m, at each occurrence, is 0 to 15;
n, at each occurrence, is 0 to 15;
o is 0 to 15; and
p is 0 to 15,
wherein linker attachment points are indicated by dotted line.

16. A bivalent compound comprising a degrader/disruption tag EL conjugated to an eleven nineteen leukemia (ENL) ligand PI via a Linker:
PI-Linker-EL,
and enantiomers, diastereoisomers and pharmaceutically acceptable salts thereof, wherein:
PI comprises the following ENL ligand: 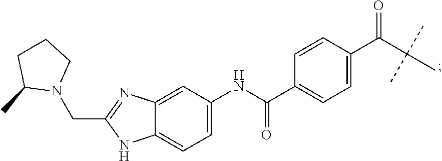
degrader/disruption tag EL is selected from the group consisting of: 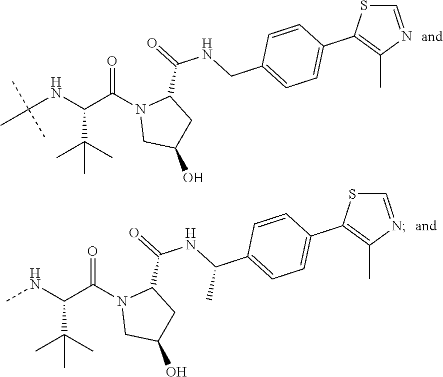
the linker is selected from the group consisting of: 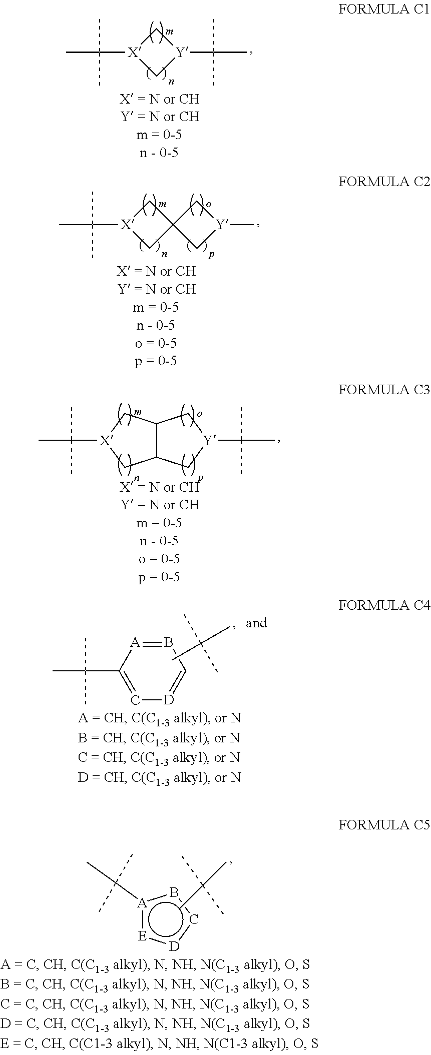
wherein linker attachment points are indicated by dotted line.

17. A bivalent compound selected from the group consisting of LQ076-105, LQ076-106, LQ076-107, LQ076-108, LQ076-109, LQ076-110, LQ076-111, LQ076-112, LQ076-113, LQ076-114, LQ076-115, LQ076-116, LQ076-117, LQ076-118, LQ076-119, LQ076-120, LQ076-121, LQ081-122, LQ081-132, LQ081-133, LQ081-147, and enantiomers, diastereoisomers and pharmaceutically acceptable salts thereof.

18. A bivalent compound selected from the group consisting of LQ108-69, LQ108-70, LQ108-71, LQ108-72, LQ126-62, LQ126-63, LQ126-81, LQ126-82, and enantiomers, diastereoisomers and pharmaceutically acceptable salts thereof.
